(19) United States
(12) Patent Application Publication
(10) Pub. No.: US 2004/0142413 A1
(43) Pub. Date: Jul. 22, 2004

(54) Isolated genome of xyllela fastidiosa and uses thereof

(76) Inventors: Andrew Simpson, New York, NY (US); Fernando Reinach, Sao Paulo (BR); Joao Setubal, Campina (BR); Joao Meidanis, Campinas (BR); Paulo Arruda, Campinas (BR)

Correspondence Address: FULBRIGHT & JAWORSKI, LLP, 666 FIFTH AVE, NEW YORK, NY, 10103-3198, US

(21) Appl. No.: 10/297,465

(22) PCT Filed: Jun. 7, 2001
(86) PCT No.: PCT/IB01/01618

(51) Int. Cl. C07H 21/04; C12N 1/21; C07K 14/195

(52) U.S. Cl. 435/69.1; 435/252.3; 435/320.1; 530/350; 536/23.7

(57) ABSTRACT

The complete genome Xyllela Fastidiosa has been isolated and sequenced. The complete sequence can be placed on various computer readable media, and used diagnostically.

### FIELD OF THE INVENTION

This invention relates to plant pathogens. More specifically, it relates to the determination and analysis of the genome of the important plant pathogen Xyllela fastidiosa, and its analysis. Various uses of the information are described as well.

### BACKGROUND AND PRIOR ART

Xyllela fastidiosa is a fastidious, xylem limited bacterium that is known to cause a variety of important plant diseases, such as citrus variegated chlorosis (“CVC”). See, e.g., Rosseti, et al, C.R. Acad. Sci. Paris série III 310:345-349 (1990). Symptoms of X. fastidiosa infection include conspicuous variegation on older leaves, with chlorotic areas on the upper sides, and corresponding light brown lesions, as well as gum like material on the lower side. Affected fruits are small, hardened, and of no commercial value.

Xyllela fastidiosa was identified as the causative agent in 1993 (Chang, et al, Curr. Microbiol 27:137-142 (1993)), and was found to be transmitted by sharpshooter leafhoppers, in 1996. (See Roberto, et al, Filopatol Bras. 21:517-518 (1996)). At present, control of the pathogen is limited to removal of infected shoots via pruning, the application of insecticides, and replacement of plants with healthy plants.

In addition to CVC, referred to supra, X. fastidiosa strains cause plant diseases such as Pierce's disease of grapevines, alfalfa dwarf, phony peach disease, periwinkle wilt, and leaf scorch of plum. The bacterium is also associated with diseases of mulberry, pear, almond, elm, sycamore, oak, maple, pecan, and coffee. See Purcell, et al, Annu Rev. Phytopathol 34:131-151 (1996). Given the broad range of pathologies with which this bacterium is associated, it would be useful to be able to diagnose or to determine presence of the bacterium in a plant, a plant part, or group of plants, such as an orchard, as well as to have available more useful approaches for eliminating the bacterium from a host.

To this end, the inventors have determined the complete genomic sequence of X. fastidiosa, as described herein. Further, the inventors have analyzed the genome, providing a map of genes within the genome. This permits the artisan to develop strategies for combatting X. fastidiosa infection, as described infra.

### BRIEF DESCRIPTION OF THE FIGURES

FIG. 1, parts a-d, depicts a map of the X. fastidiosa chromosome. The figure is best read by placing panels a-d in a horizontal array, with panel a at the left, moving to the right, with d as the last panel. Each arrow indicates an individual gene within the chromosome. FIG. 2 depicts the biochemical processes involved in X. fastidiosa pathogenicity and survival in host xylem. Major functional categories are in bold, with bacterial genes and gene products related to that function indicated with the bold heading. In FIG. 2, the following code is employed:

cylinders are channels;

ovals are secondary carriers, including the MFS family;

paired dumbbells are secondary characters for drug extrusion triple dumbbells are ABC transporters the bulb-like icon is an F-type ATP synthase;

squares are other transporters.

Any icon with two arrows indicates symporters and antiporters (these are H⁺ or Na⁺ porters, unless otherwise noted).

### DETAILED DESCRIPTION OF PREFERRED EMBODIMENT

Sequence information as described herein may also be found in the GenBank database, under the following accession numbers: AE003849 (the chromosome); AE003850 (plasmid pXF1.3), and AE003851 (plasmid pXF51).

### EXAMPLE 1

The experiments which follow utilized triply cloned X-fastidiosa 9a5c, which was derived from a pathogenic culture referred to as 8.1b, that had been obtained in 1992 in Bordeaux, France, from CVC affected Valencia sweet orange twigs that had been collected in Macaubal, Sao Paolo, Brazil, on May 21, 1992. See Chang, et al, Curr. Microbiol. 27:137-142 (1993). This strain produces typical CVC symptoms upon inoculation of citrus plants (Li, et al, Curr. Microbiol 39:106-108 (1999)), and has also produced such symptoms in Nicotania tabacum and Catharantus roseus.

A combination of ordered cosmid and shotgun strategies was used, in accordance with Fleischmann, et al, Science 269:496-512 (1995), incorporated by reference. This technique has been used to sequence and assemble the entire genome of H. influenzae. See, e.g., EP 0821737 to Fleischmann et al, as well as WO9633276, both of which are incorporated by reference. The resulting cosmid library provided approximately 15-fold genome coverage, including 1056 clones, having an average insert size of 40 kilobase pairs.

High density colony filters of this library were made, and a physical map was constructed, in accordance with Hoheisel, et al, Cell 73:109-120 (1993), using a strategy of hybridization without replacement. The Hoheisel reference is incorporated by reference.

A total of 113 cosmid clones were selected for sequencing based upon the hybridization map and end sequence analysis.

Cosmid sequences were assembled into 15 contigs, covering about 90% of the genome. Further, shotgun libraries, with insert sizes ranging from 0.8 to 2.0 kilobase pairs, and 2.0 to 4.5 kilobase pairs were constructed from nebulized or restricted genomic DNA cloned into plasmids. These were sequenced to achieve 3.74 fold coverage of high quality sequence

(29,140) reads). Most of this sequencing was performed with “Big Dye” terminators, on “ABI Prism 377” DNA sequences.

Cosmid and shotgun sequences were assembled into 6 contigs, and then sequence gaps were identified by linking information from forward and reverse reads, and closed by one of primer walking or insert subcloning.

Remaining gaps were closed via combinatorial PCR, and by lambda clones selected from a λ DASH library, via end sequencing.

Colinearity between the genome and obtained sequence was confirmed via digestion of the genomic DNA with AscI, NotI, SfiI, SmiI and SrfI, followed by comparison of the digestion pattern with the electronic digestion of the generated sequence.

Further, sequences from both ends of the majority of cosmid clones, and 236 λ clones were used to confirm orientation and integrity of contigs. Gordon, et al, Genome Res 8:195-202 (1998), incorporated by reference, was used for the assembly. All consensus bases were of high quality, with Phred values of at least 20. No unexplained high quality discrepancies were found, and each consensus base was conformed by at least one read from each strand. Overall error estimate was less than one in every 10,000 bases.

### EXAMPLE 2

Following the work described in example 1, open reading frames (“ORFs”) were analyzed, in accordance with Delcher, et al, Nucleic Acid Res 27: 4636-4641 (1999), incorporated by reference. A few ORFs were determined by hand, using Altschul, et al, Nucleic Acids Res 25:3389-3402 (1997), also incorporated by reference. Annotation was facilitated, via comparison to public databases, using BLAST (Altschul, et al, supra), tRNAscan-SE (Lowe, et al, Nucleic Acids Res 25:955-96A (1997), and the functional categories for E. coli, described by Riley, et al, Microbiol Rev 57:862-952 (1993), also incorporated by reference.

In the specific category of transport proteins, ORFs were compared to the custom database found at http://www.biology.ucsd.edu/˜msaier/transport/toc.html, and phylogenetic trees for conserved “COGS” (Tatusov, et al, Nucleic Acids Res 28:33-36 (2000)), were built using the methods of Thompson, et al, Nucleic Acids Res 25:4876-4882 (1997), and Felsenstein, et al, Cladistics 5:164-166 (1989). These three references are all incorporated by reference.

Paralogous gene families were then determined in accordance with BLASTX, using an E value cut off equal to e-5, such that at least 60% of the query sequence, and at least 30% of the subject sequence were aligned.

What follows is an analysis of the genome.

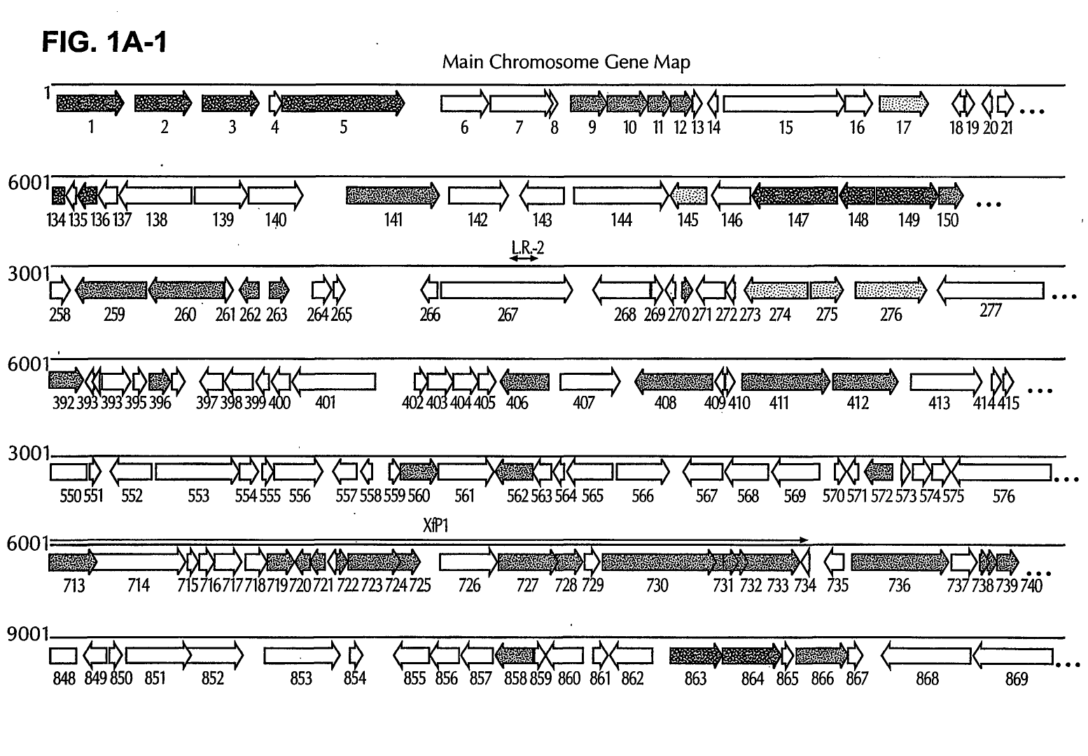

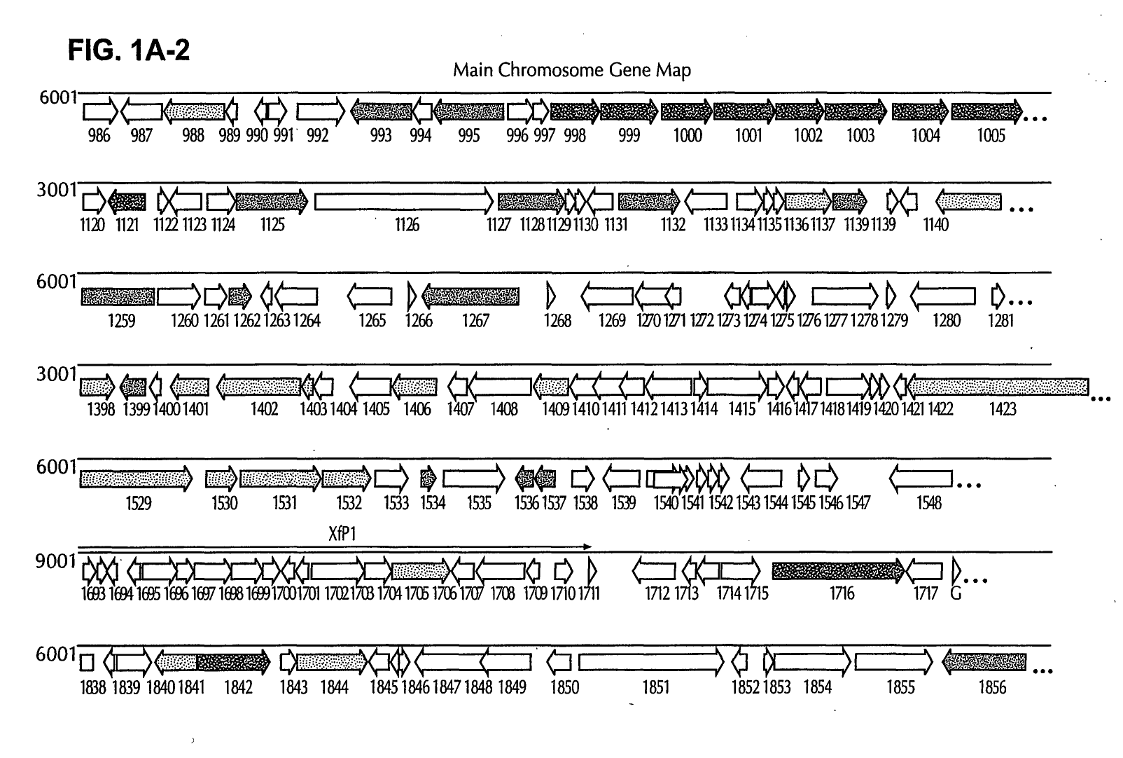

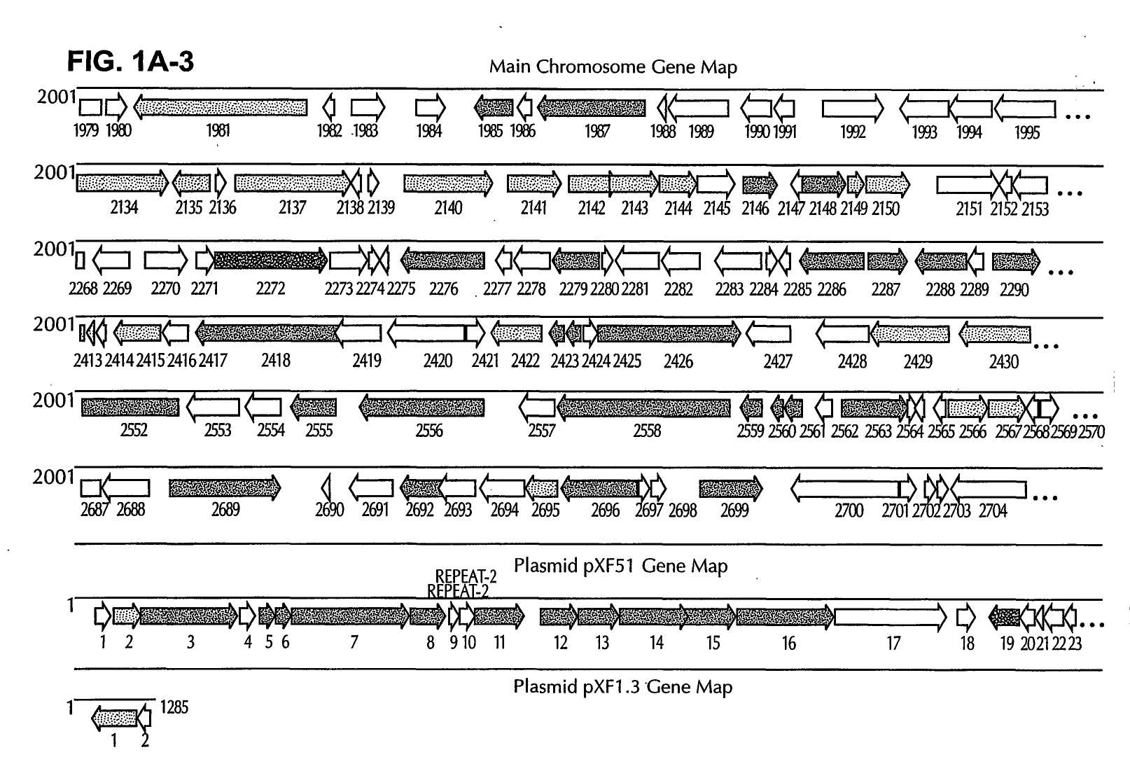

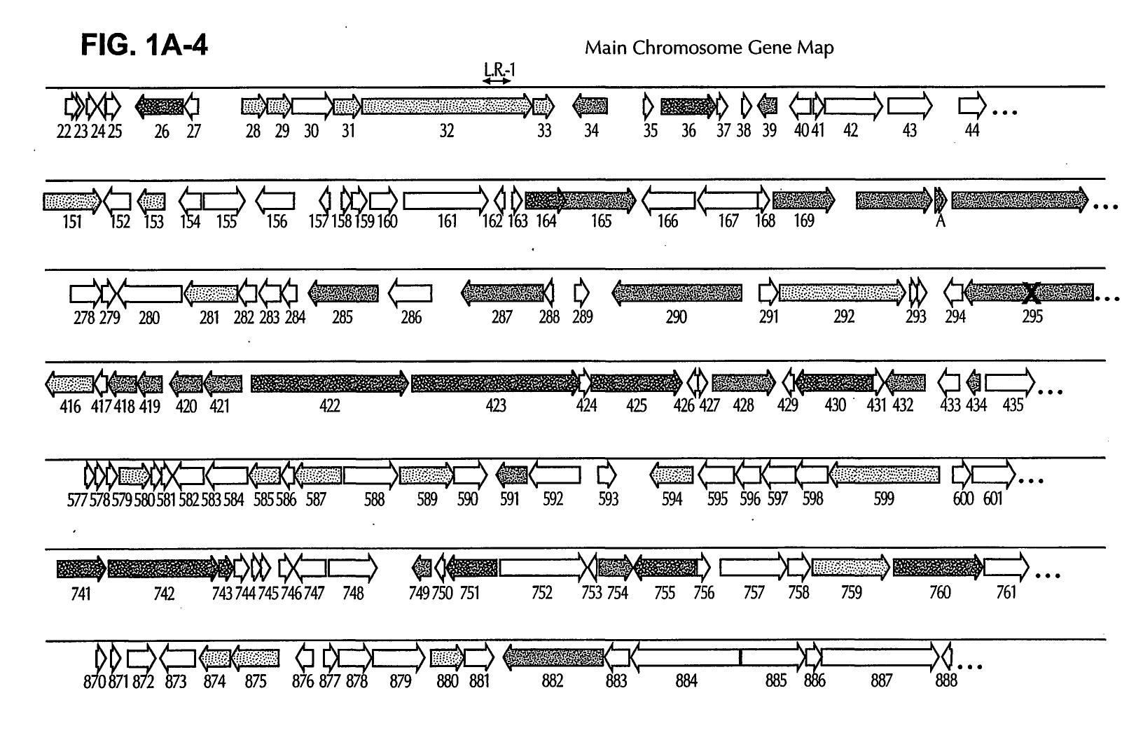

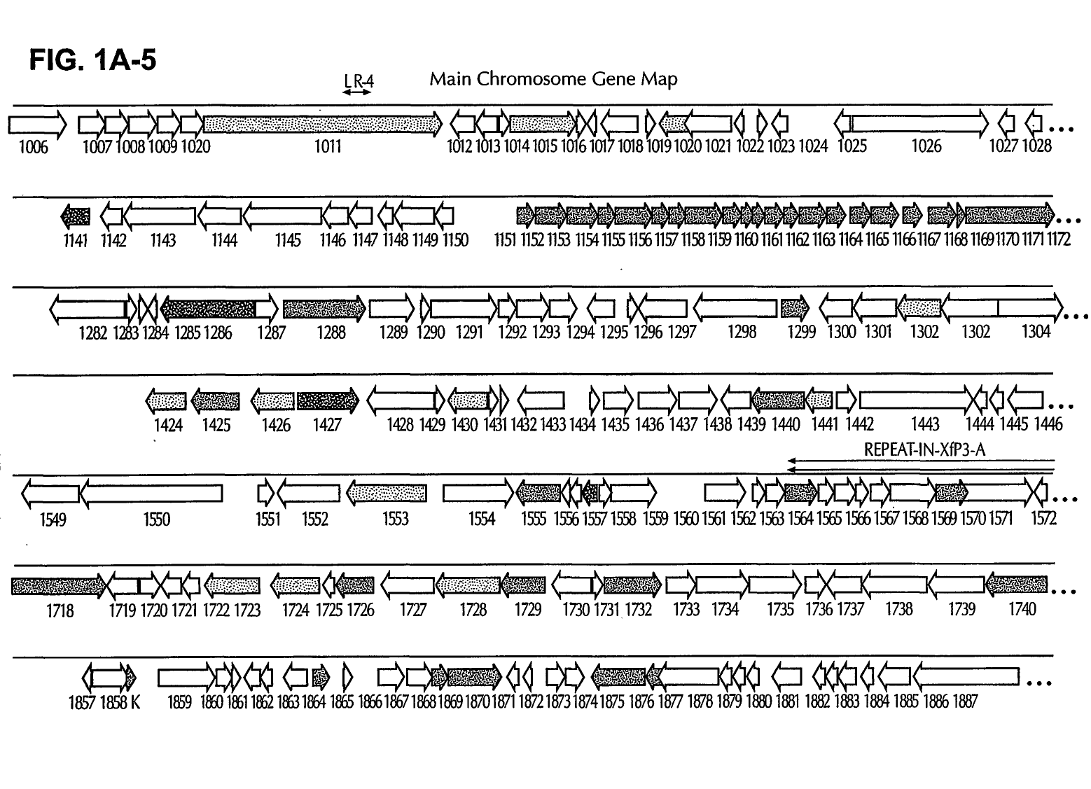

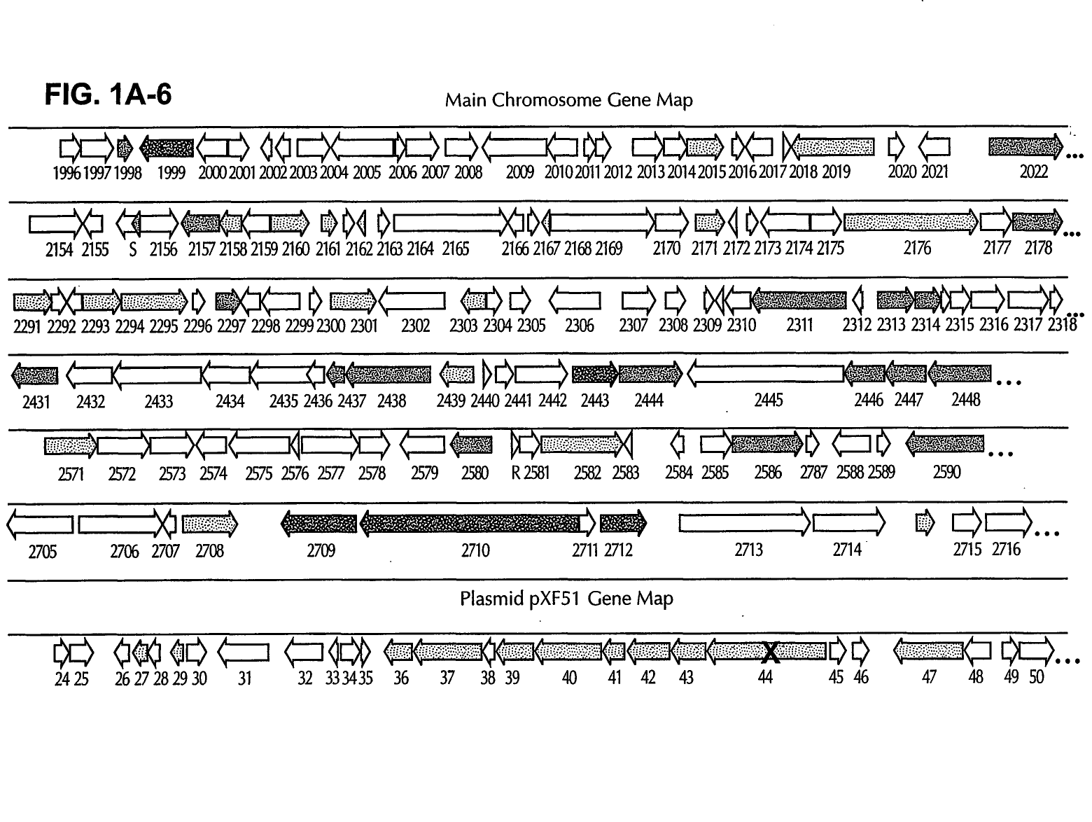

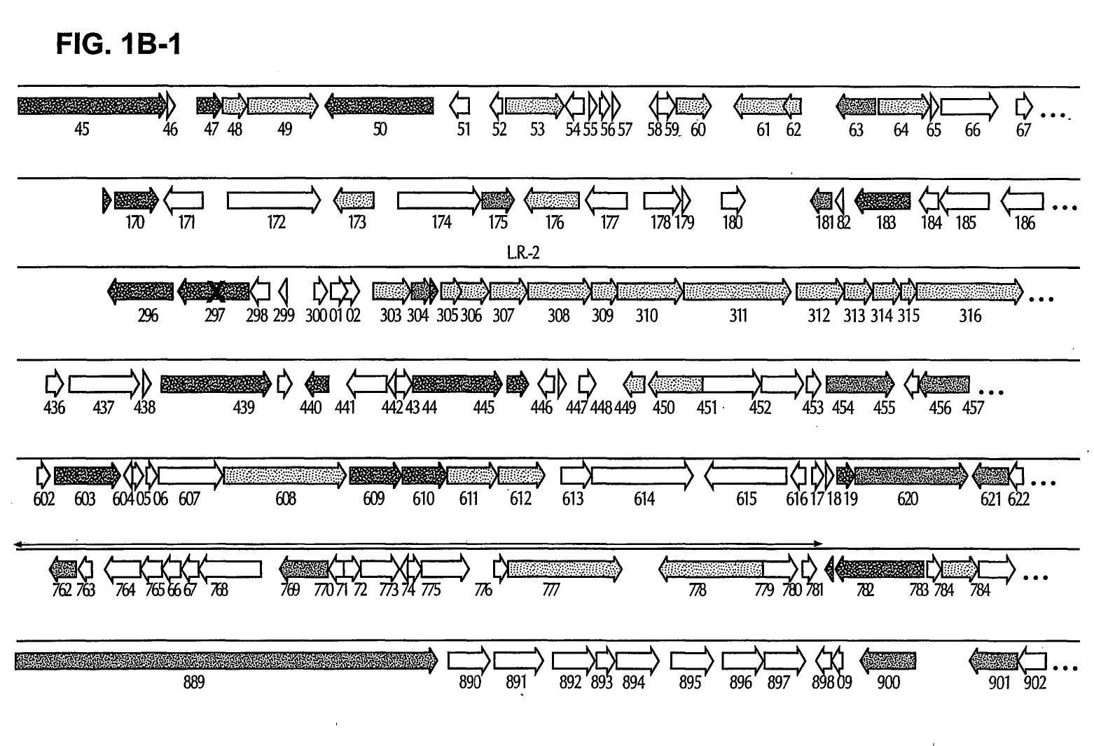

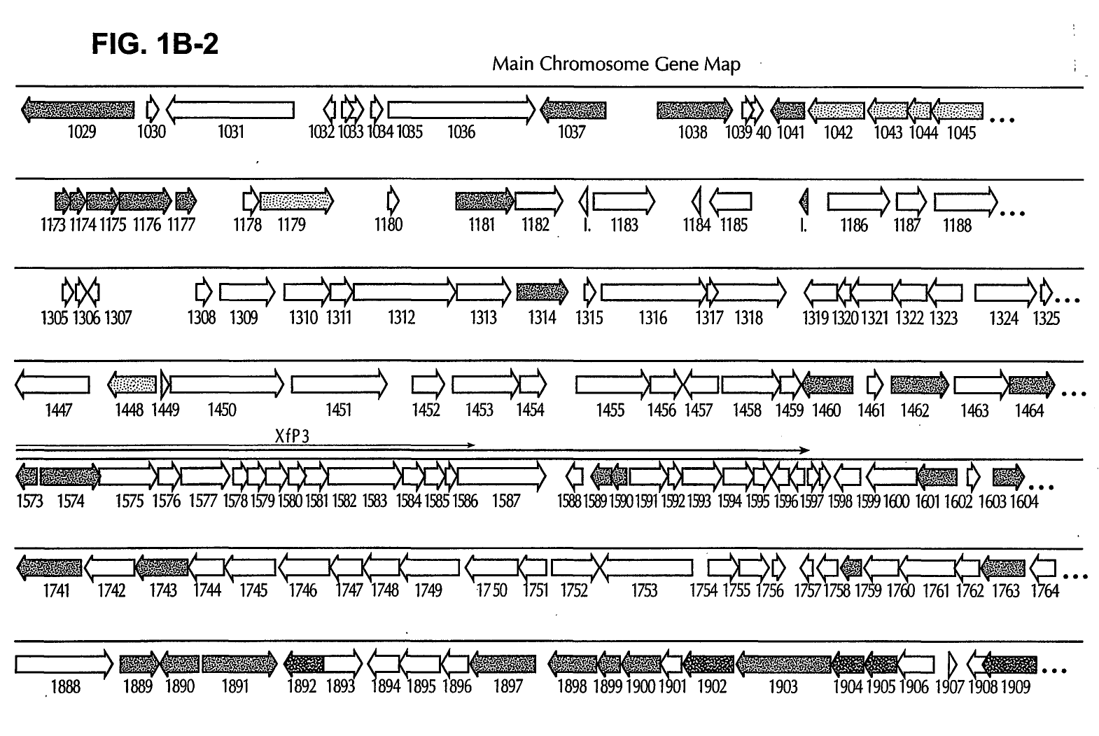

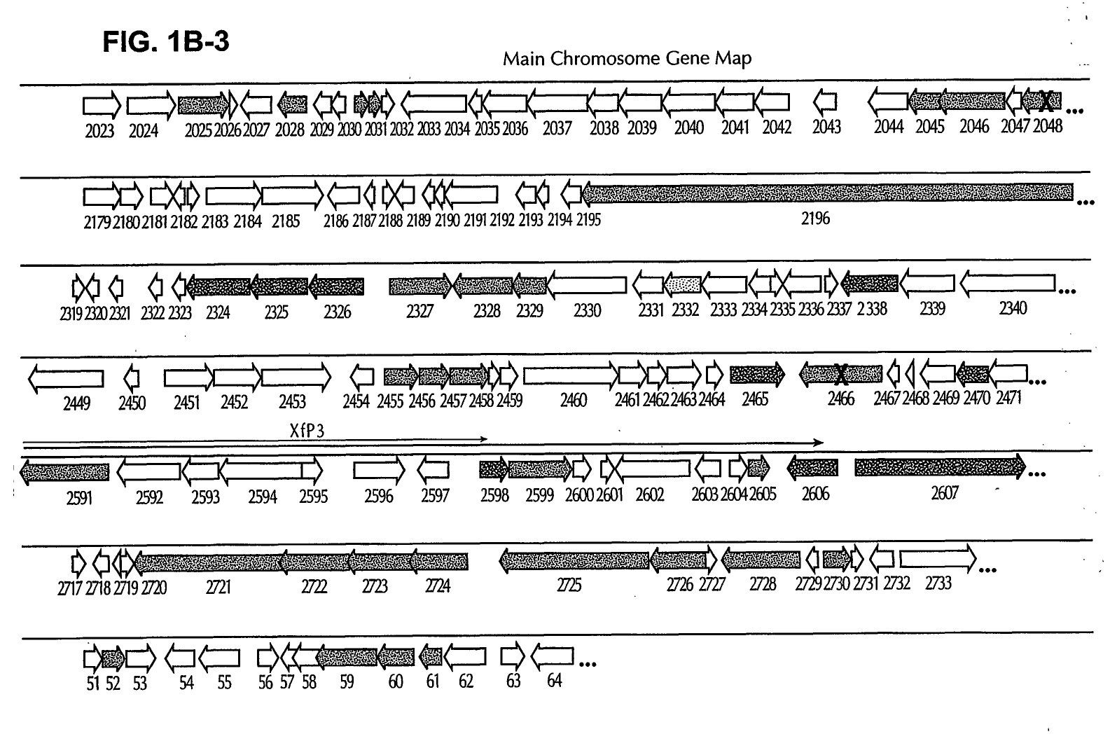

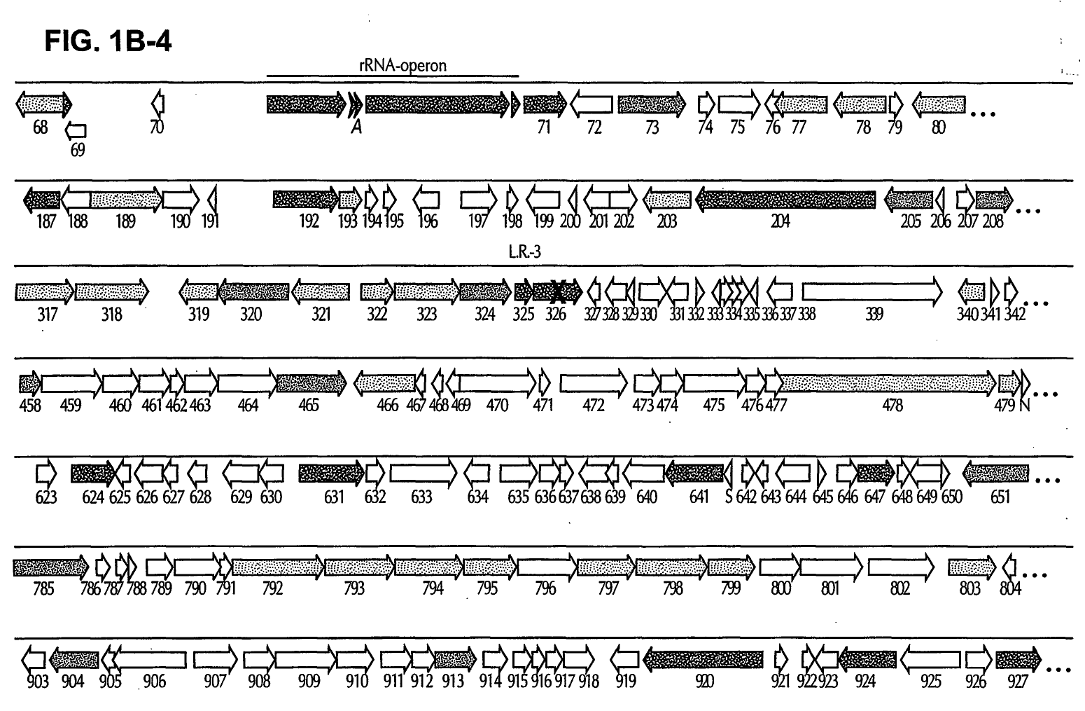

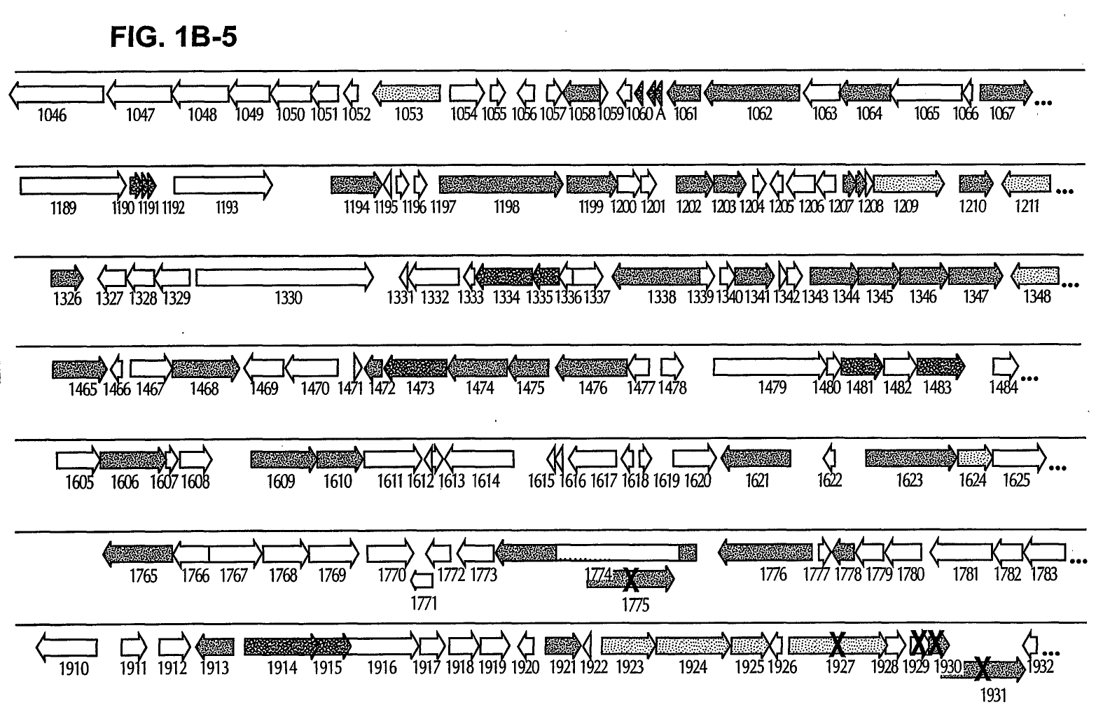

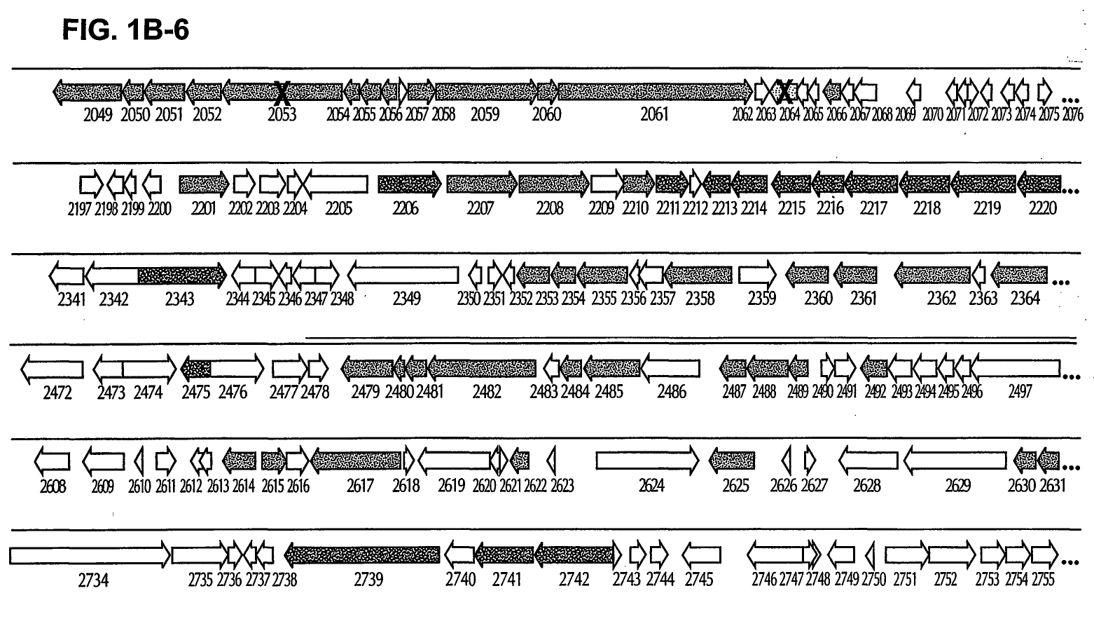

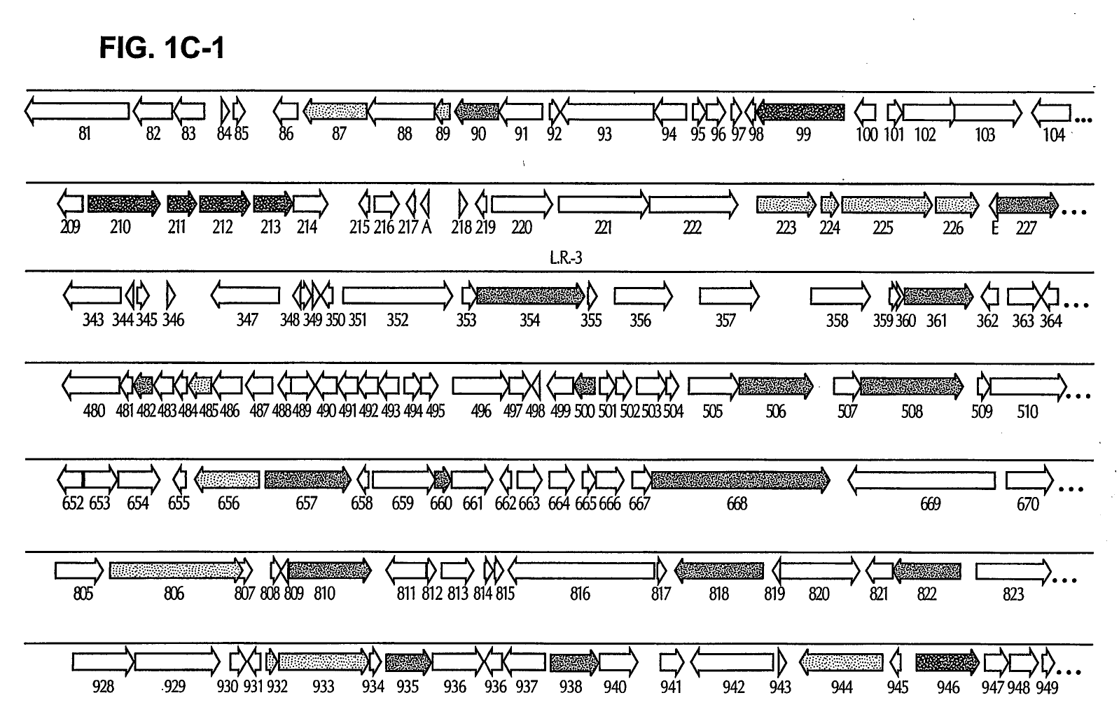

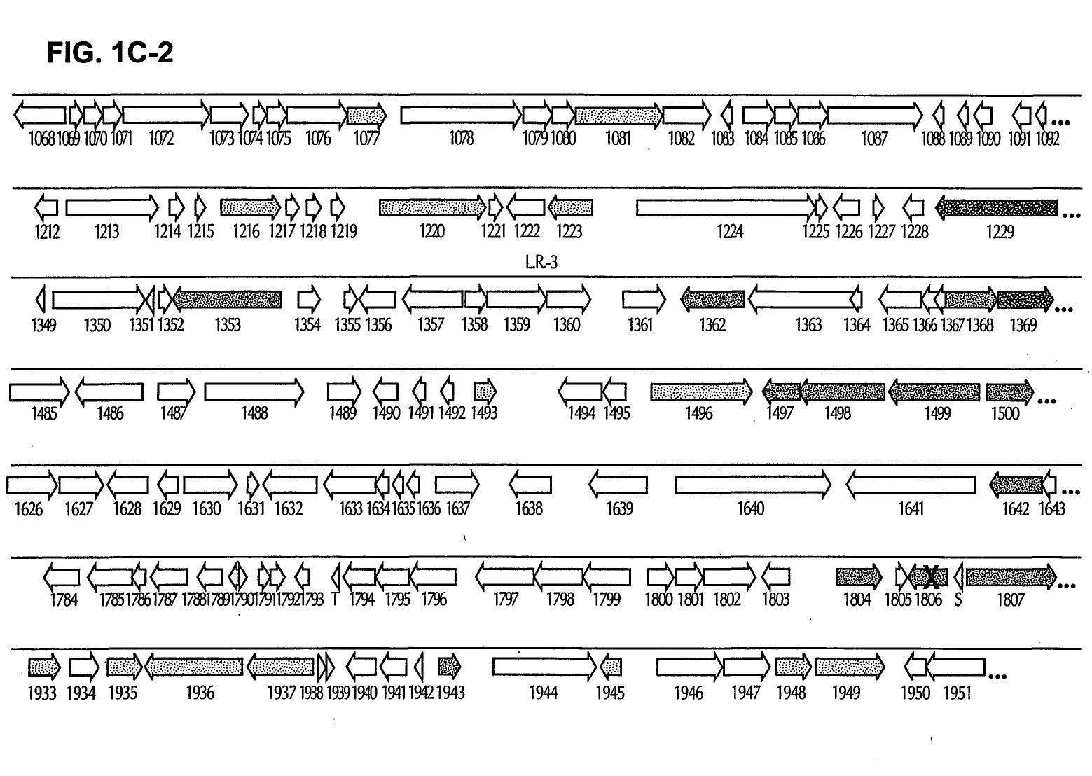

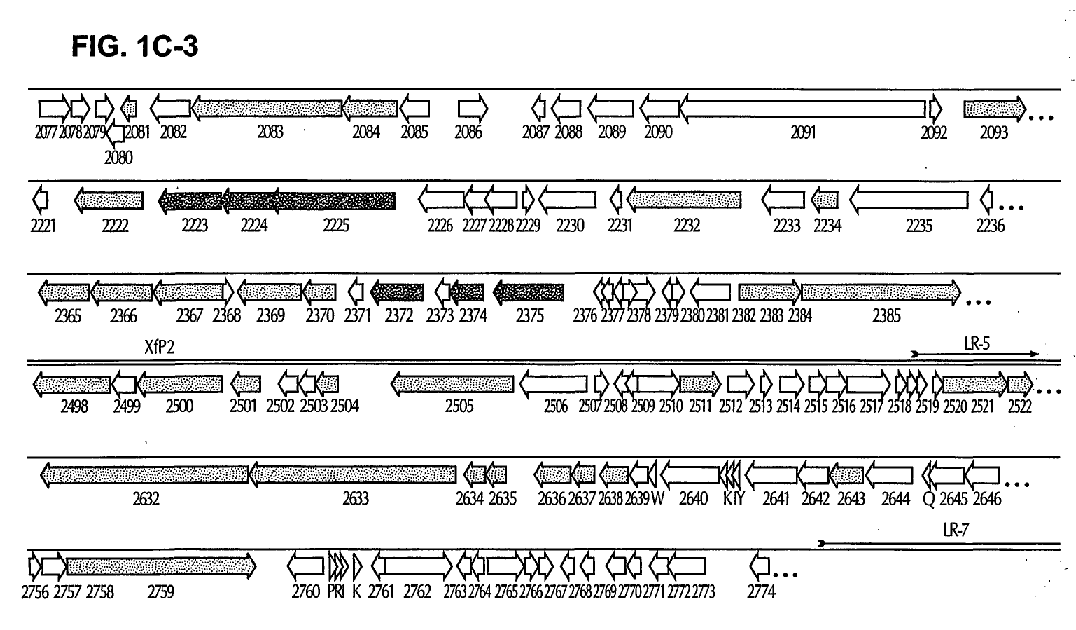

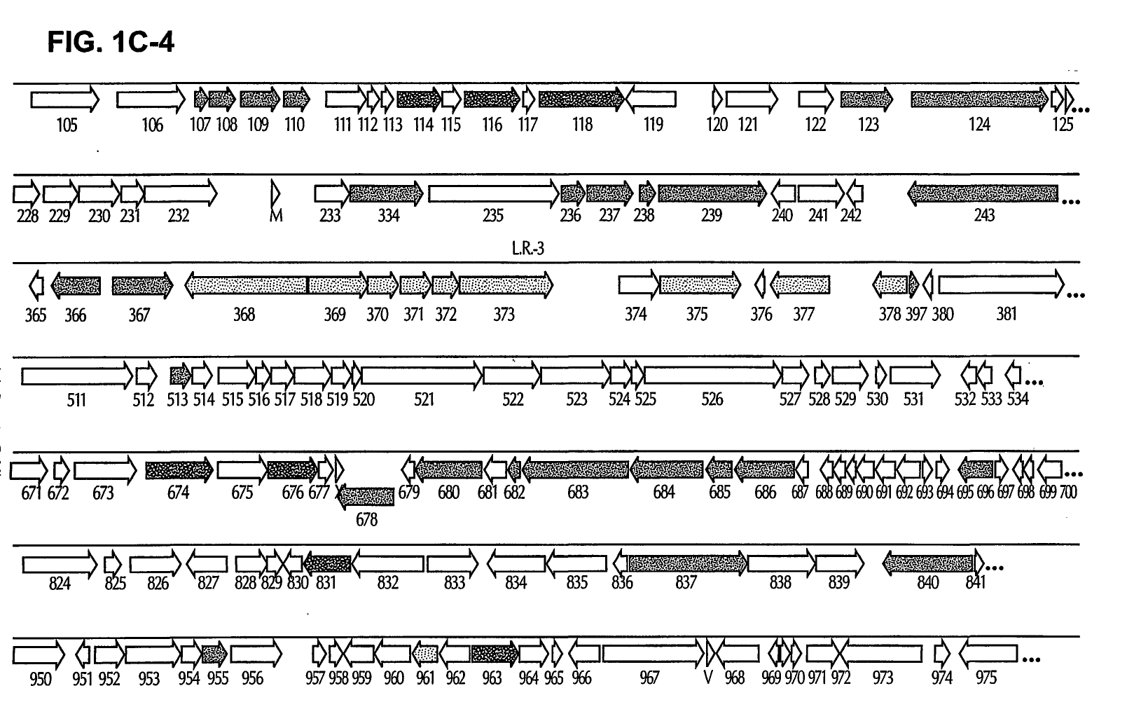

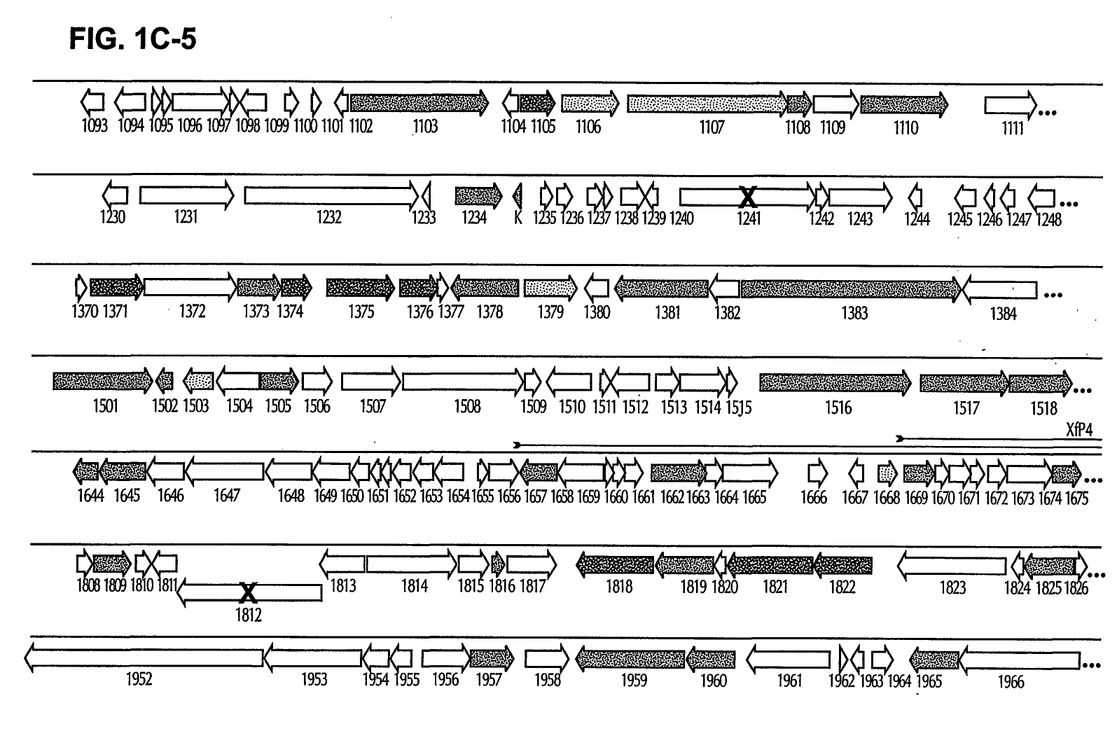

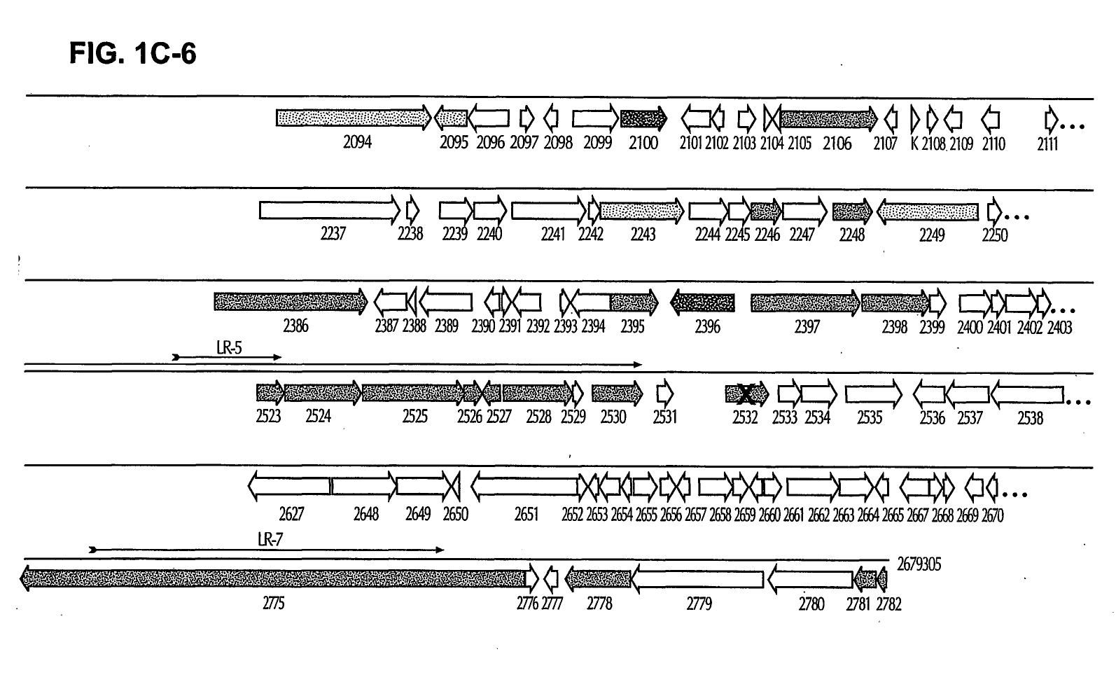

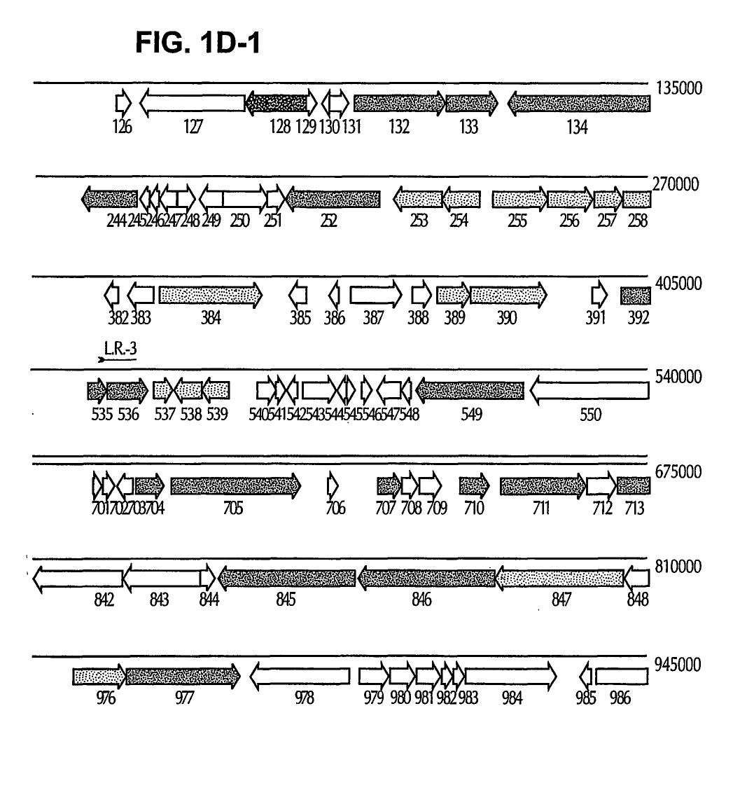

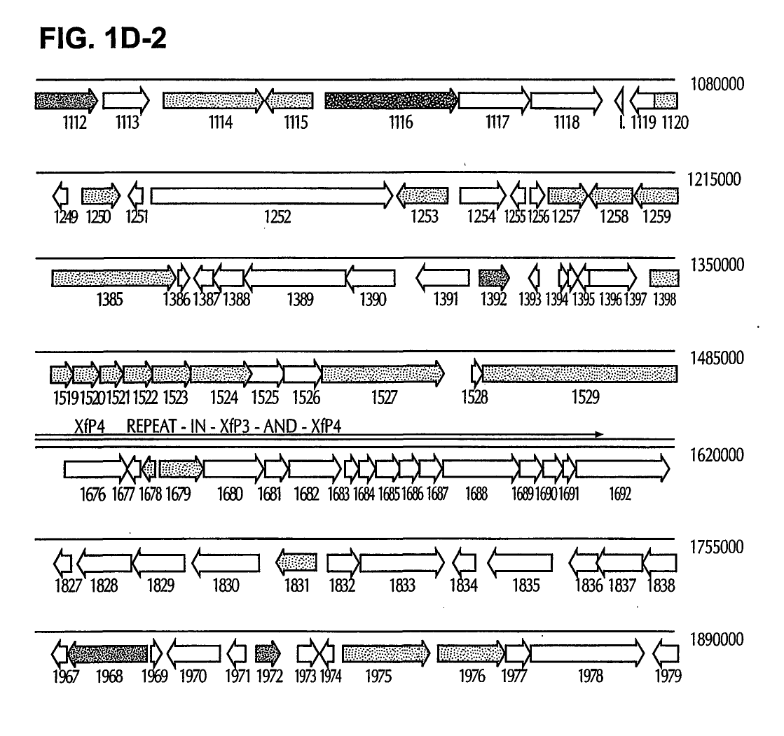

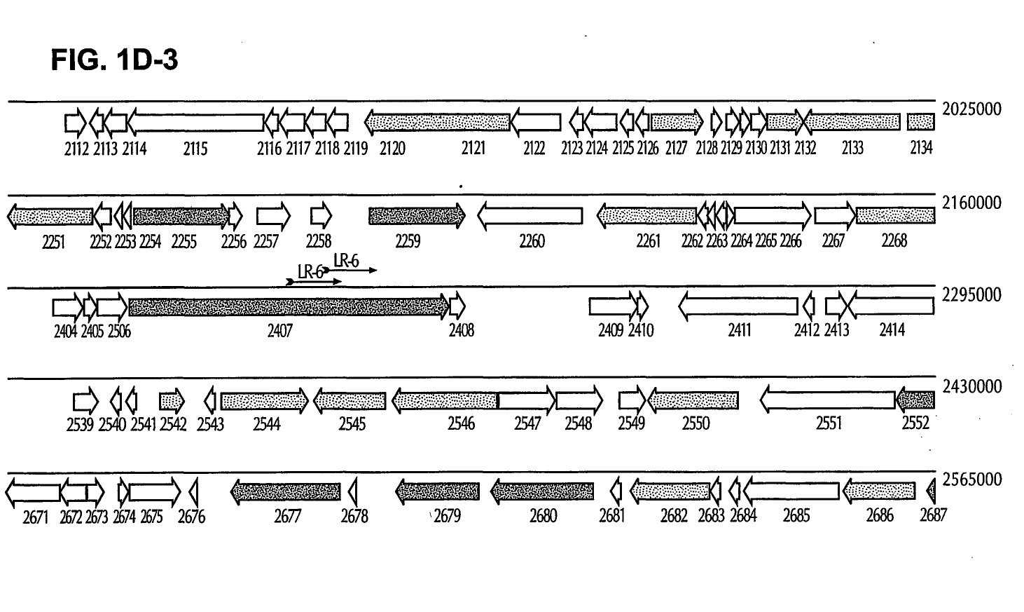

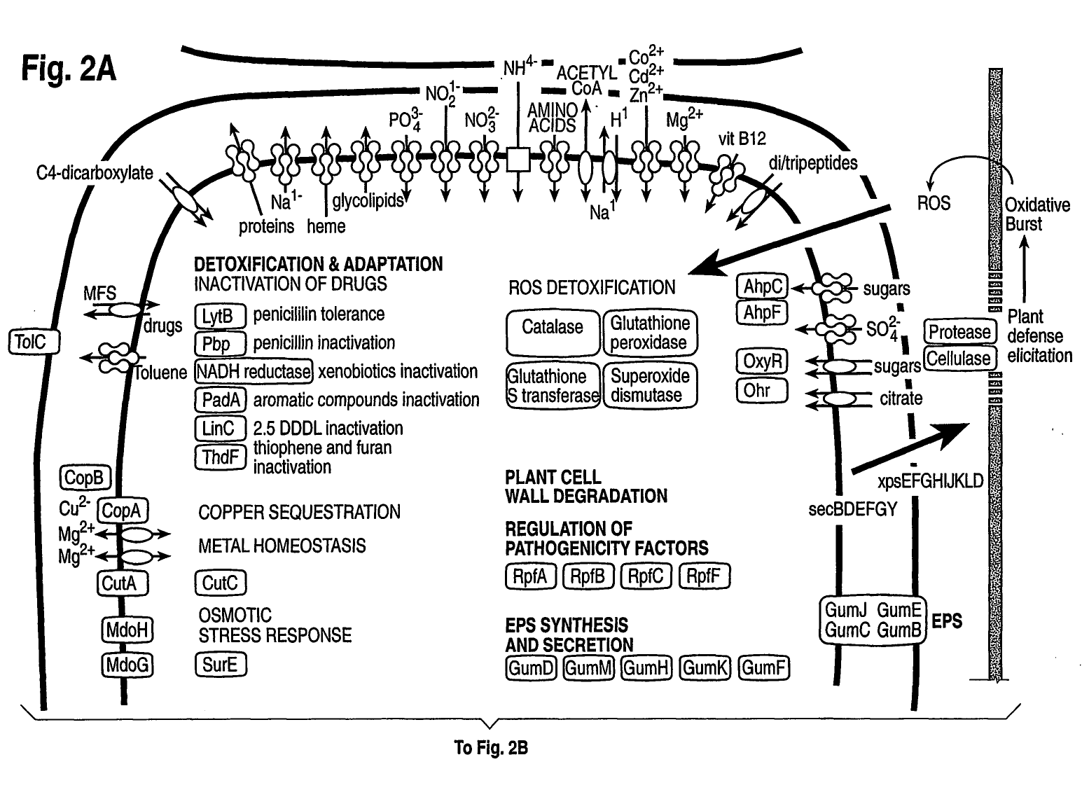

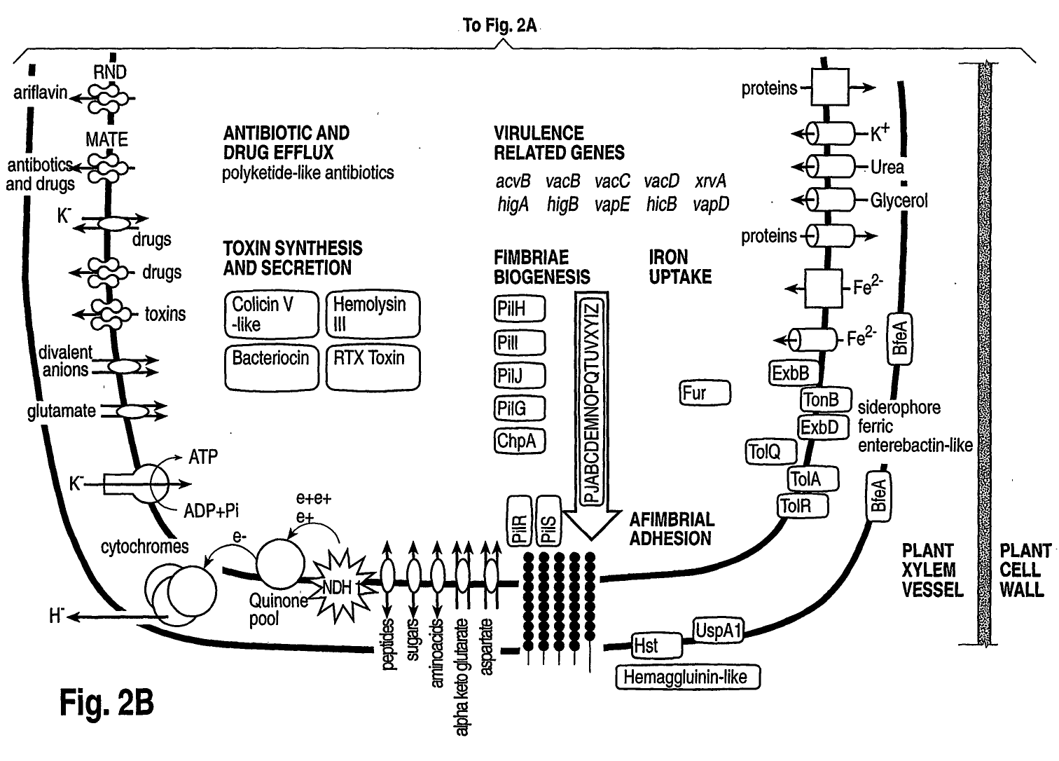

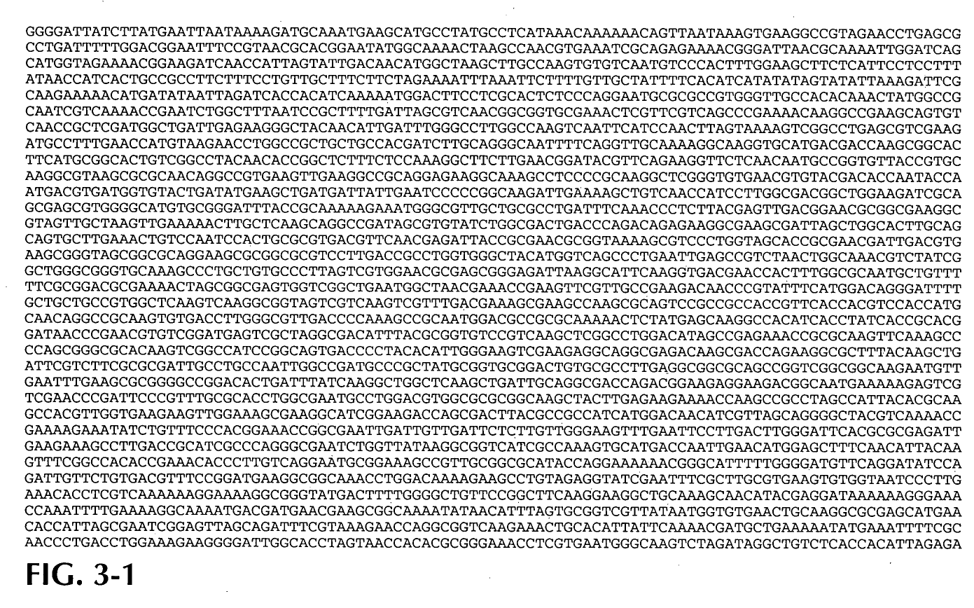

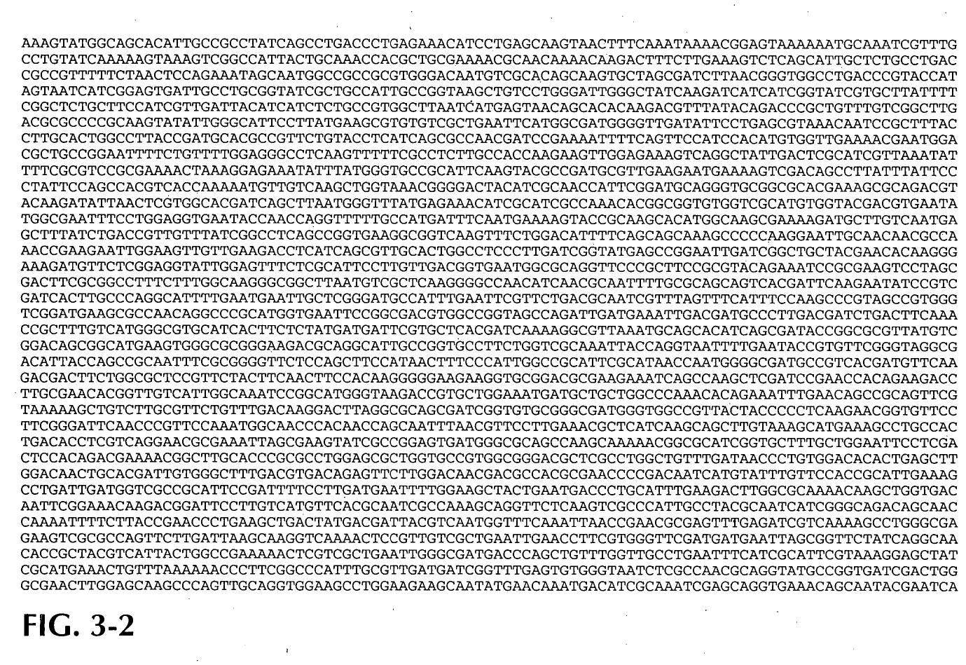

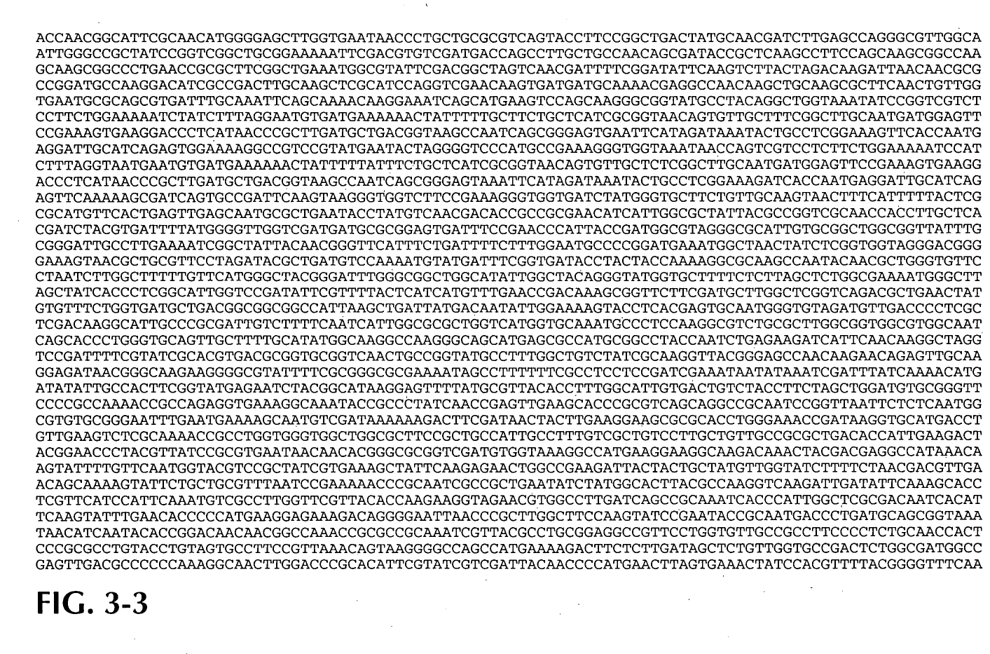

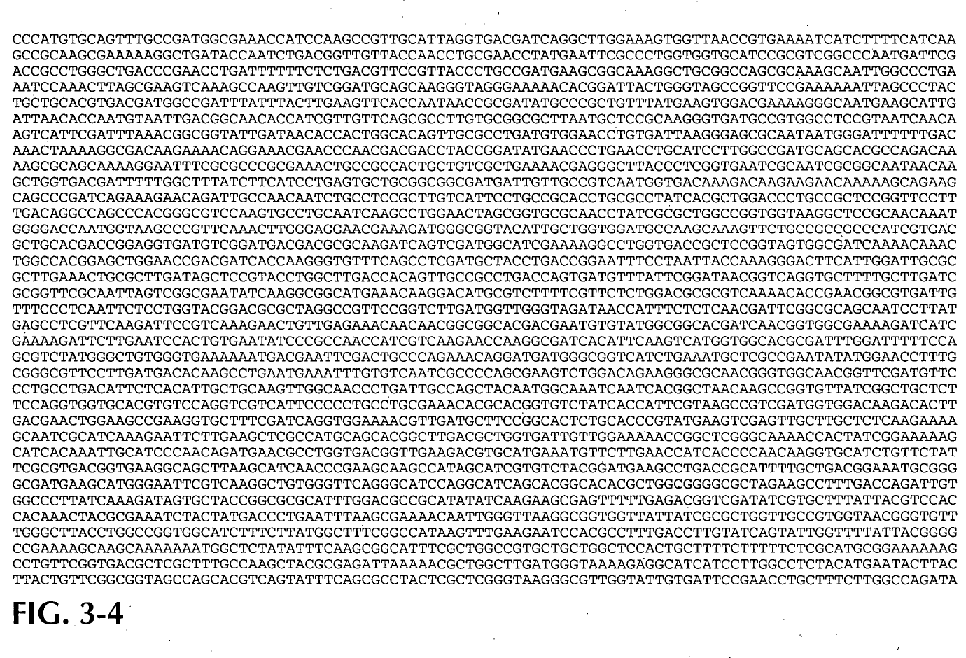

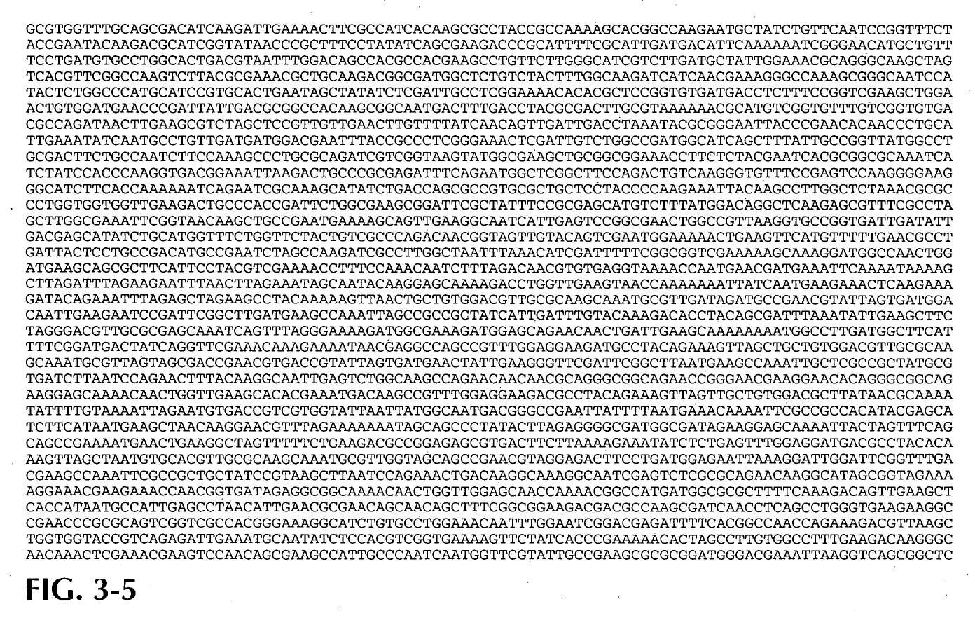

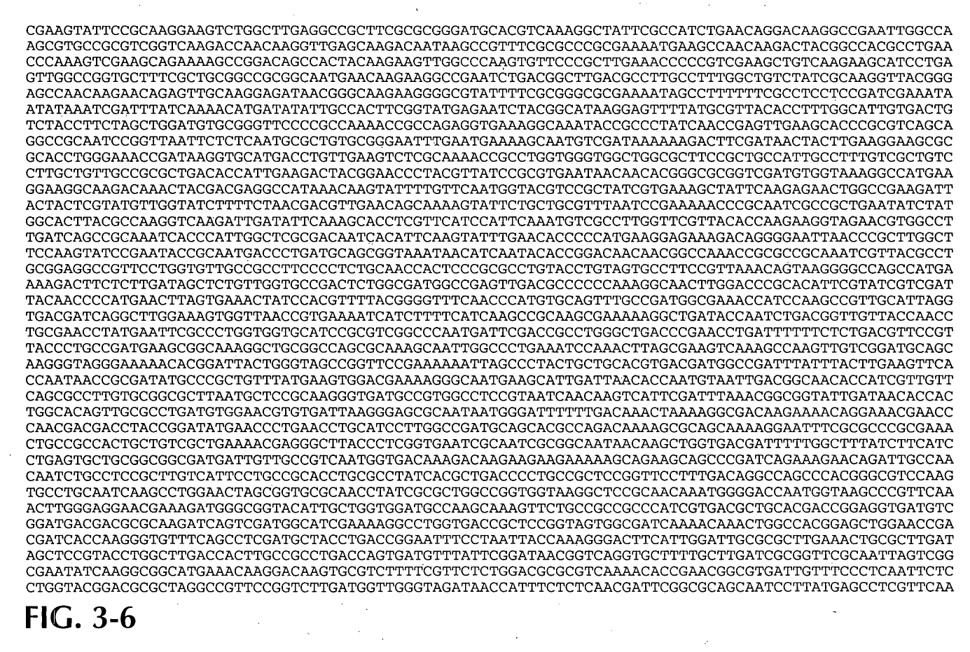

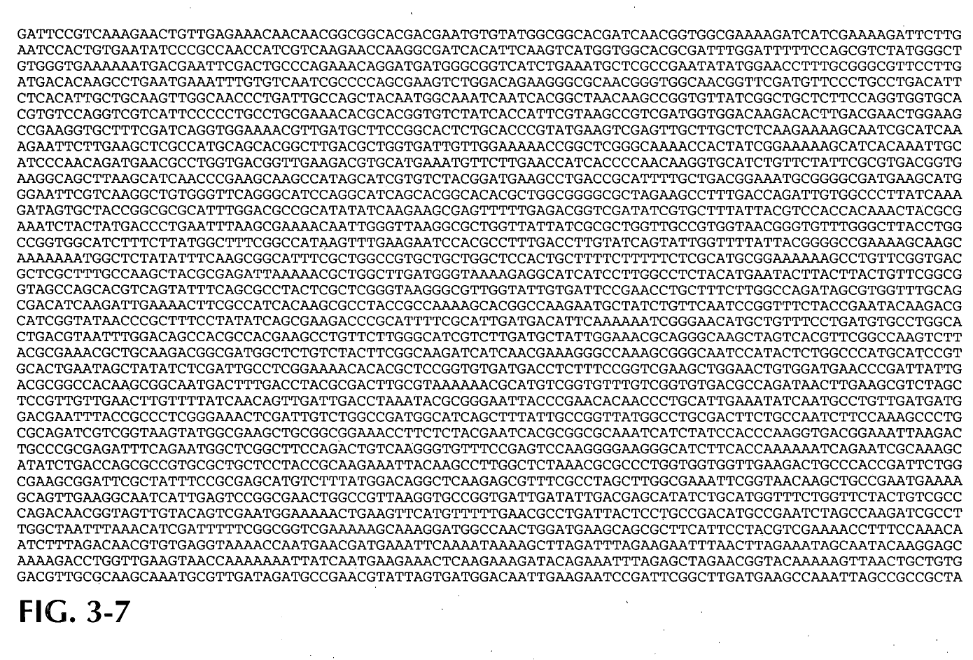

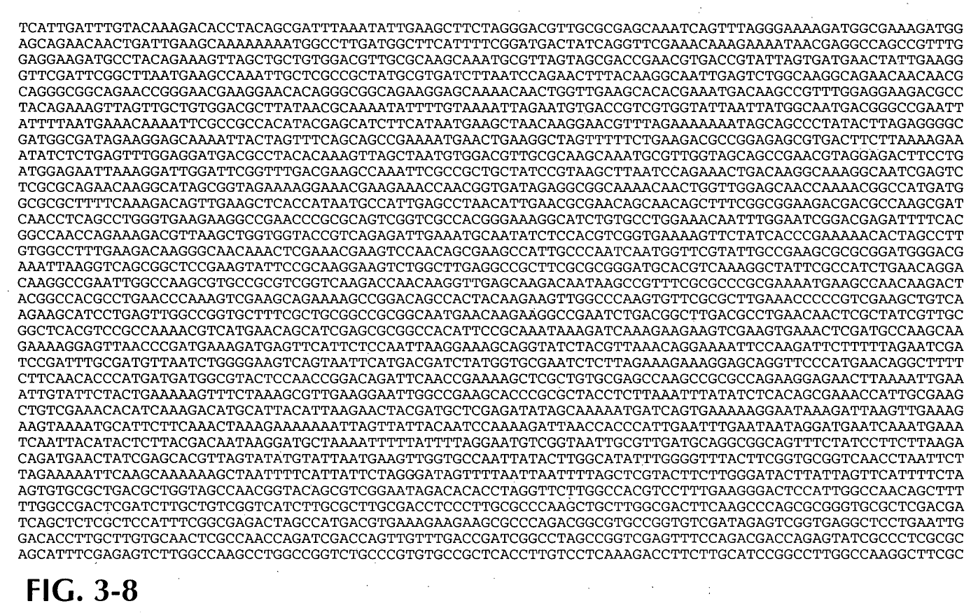

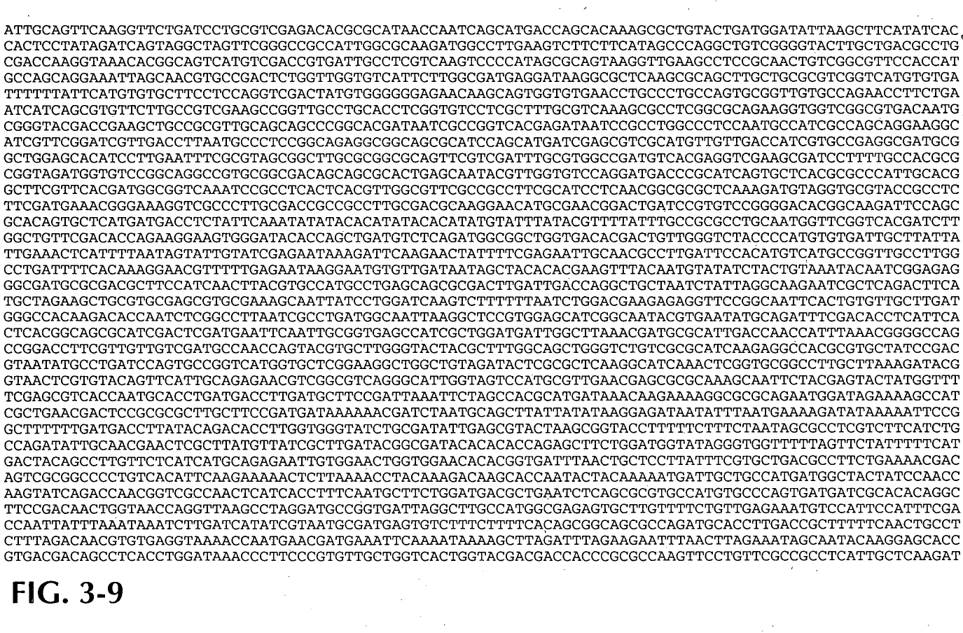

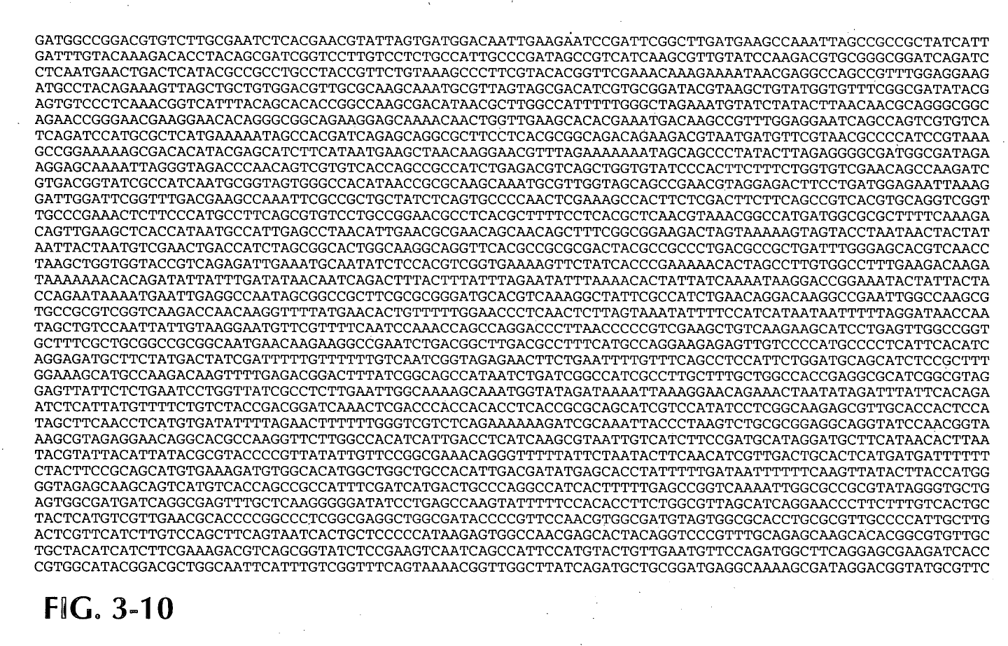

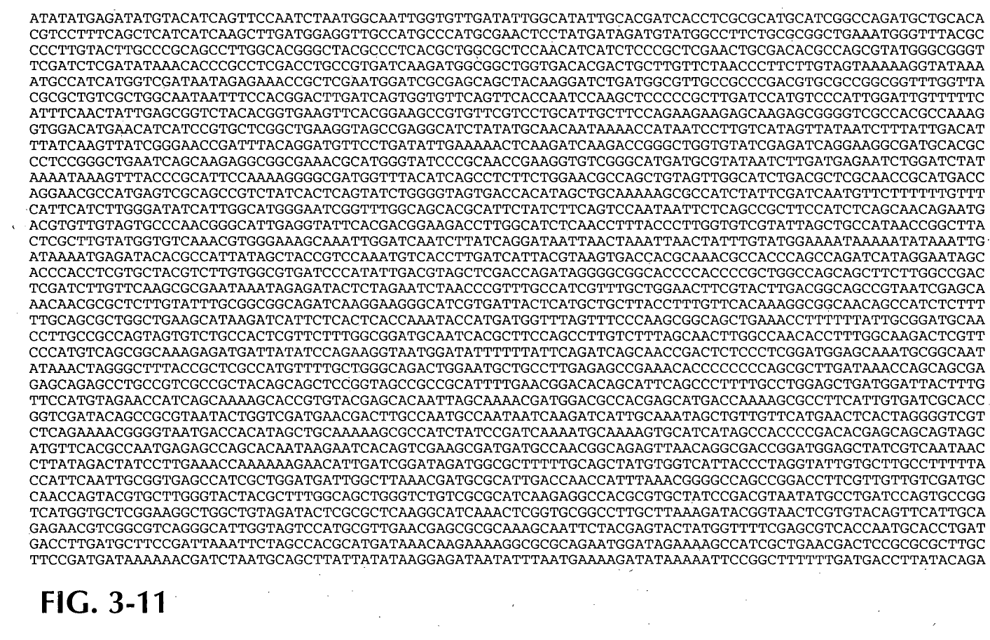

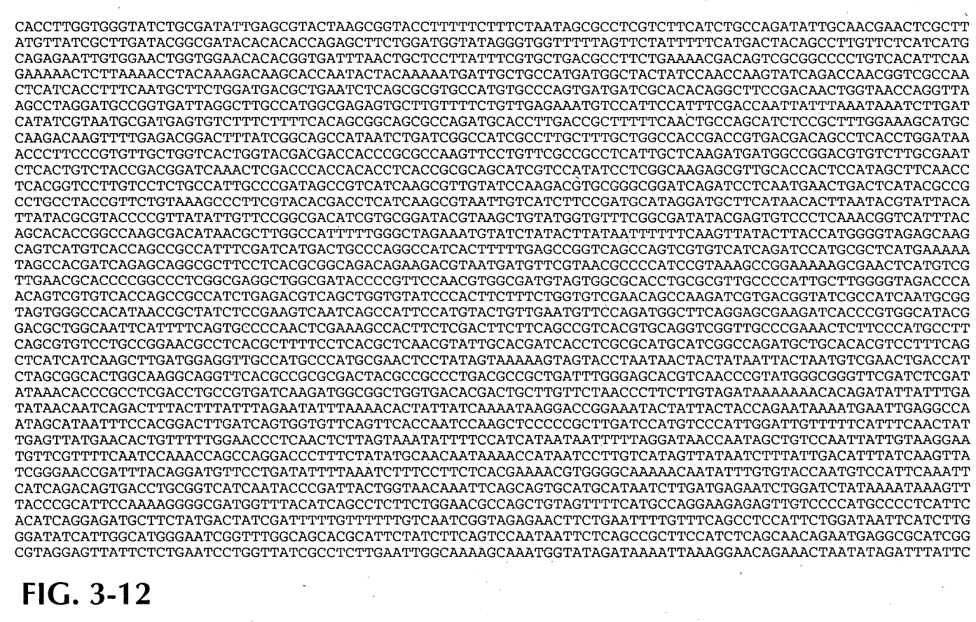

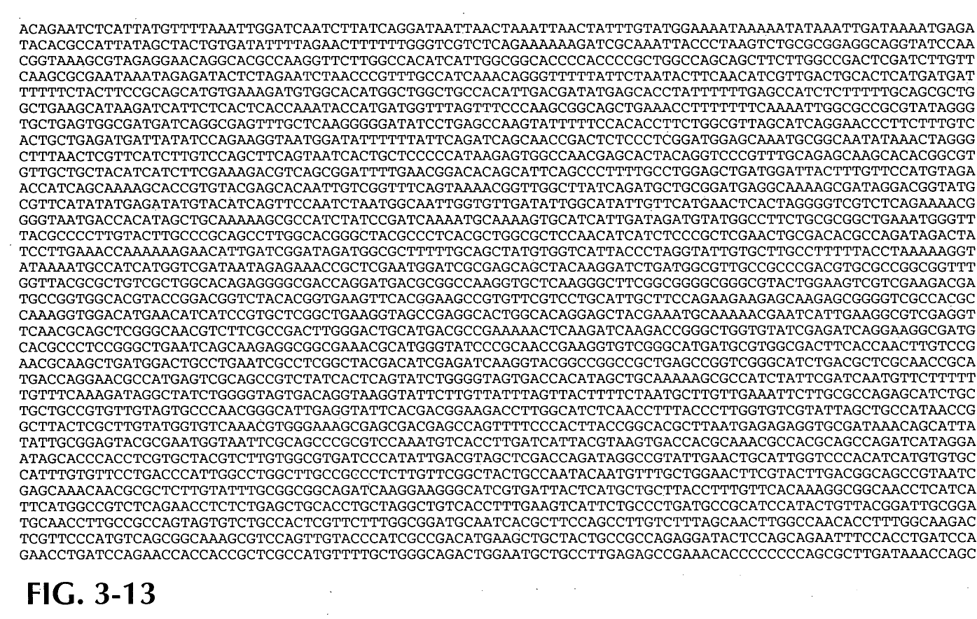

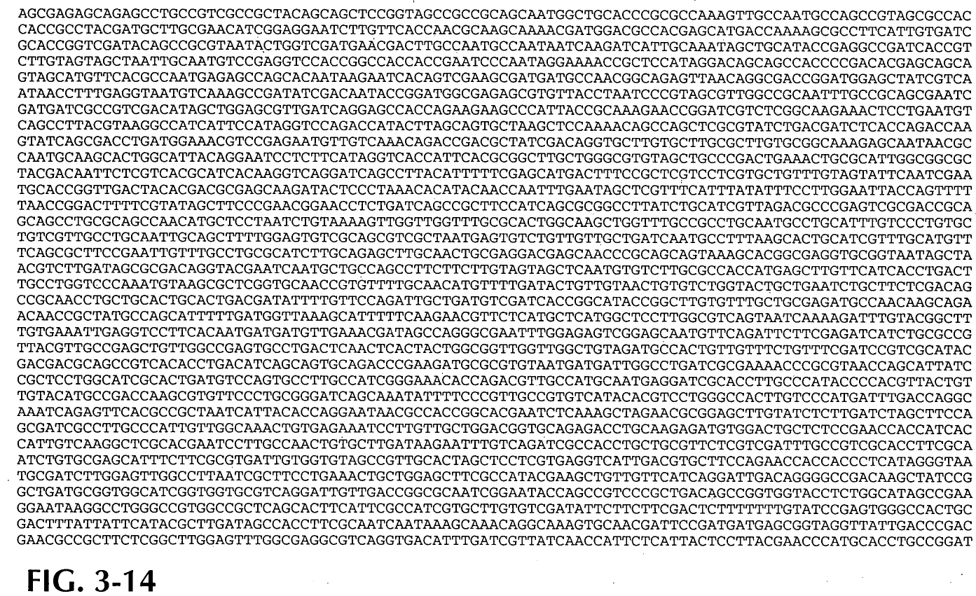

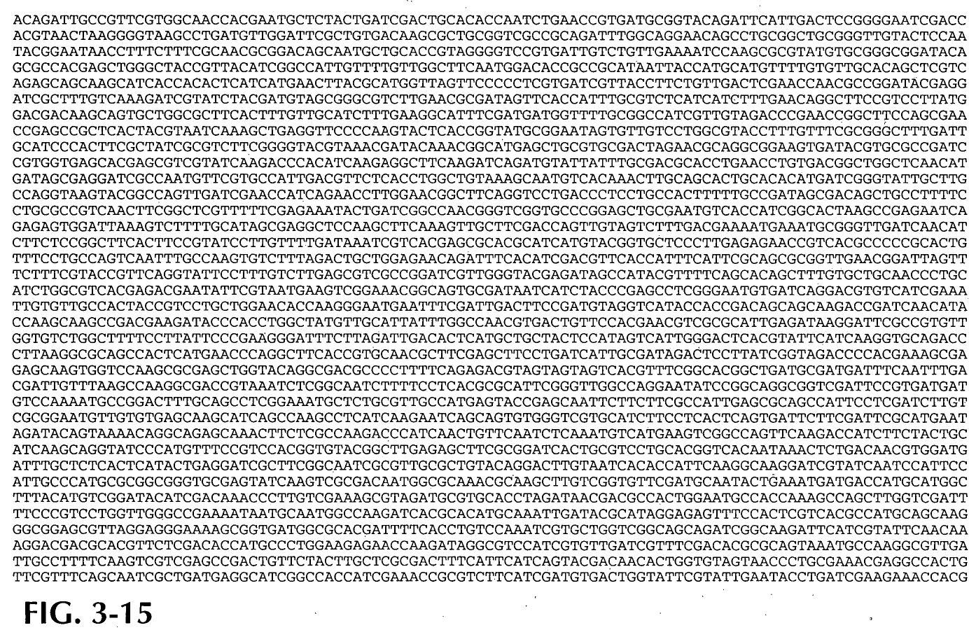

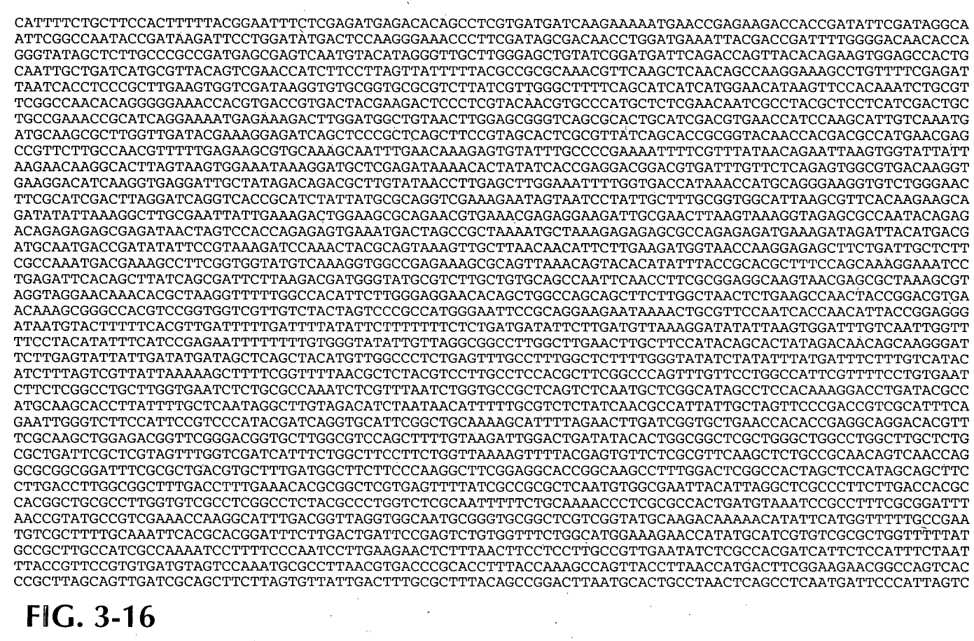

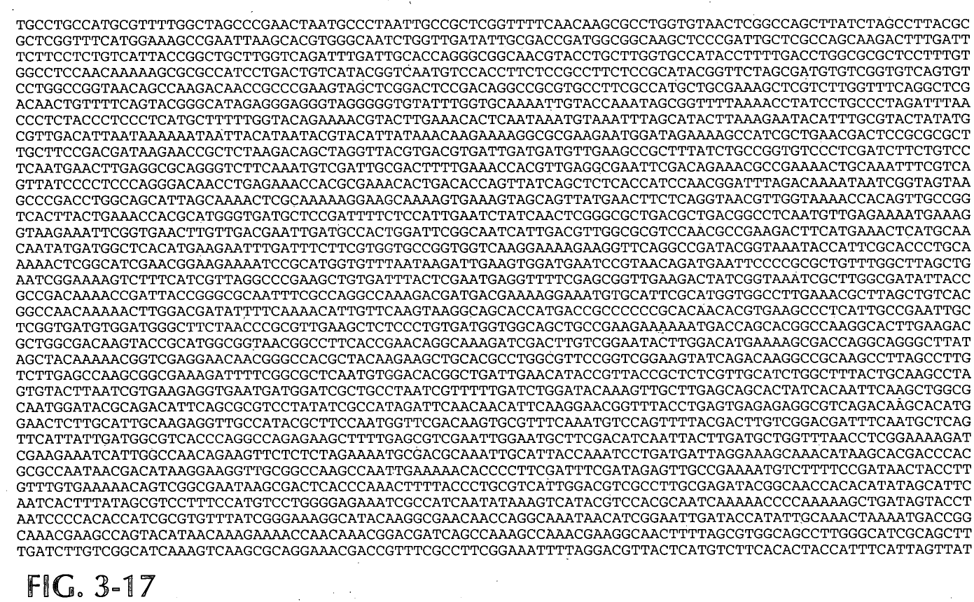

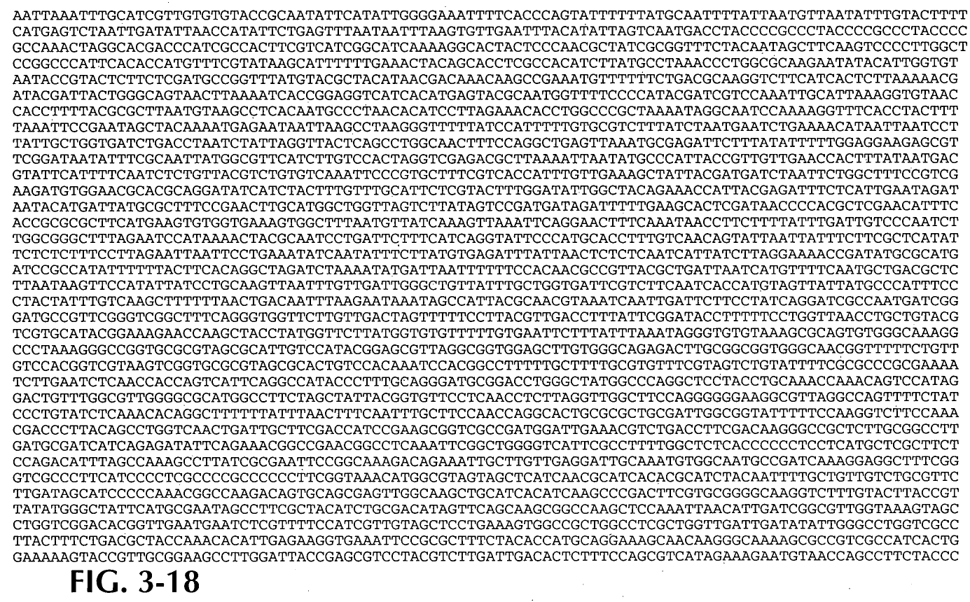

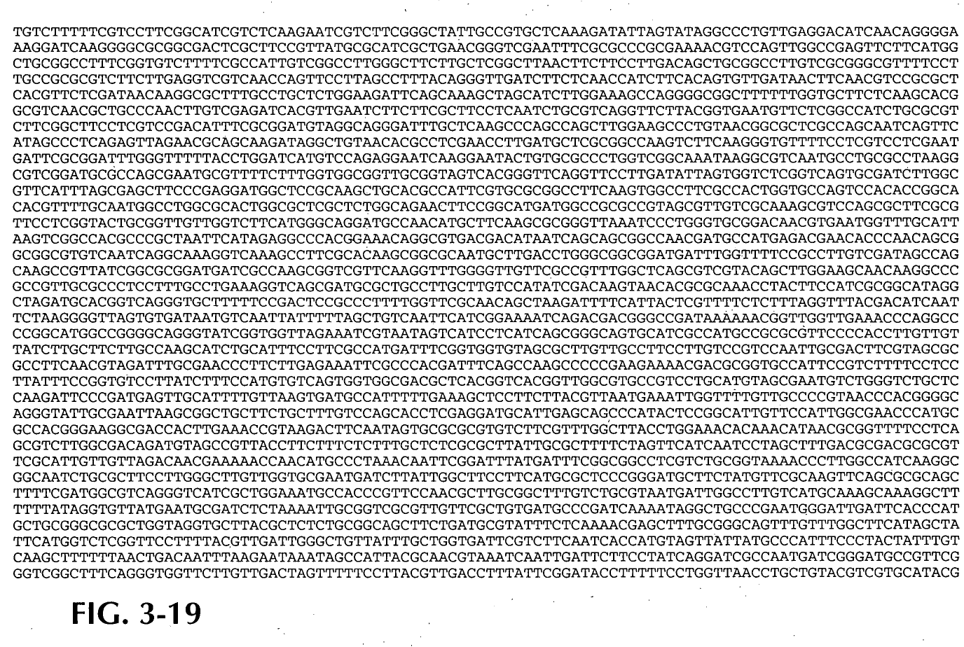

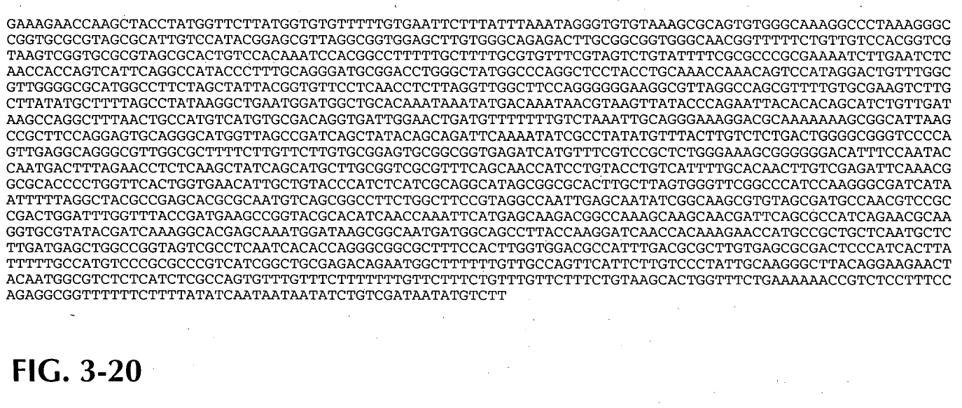

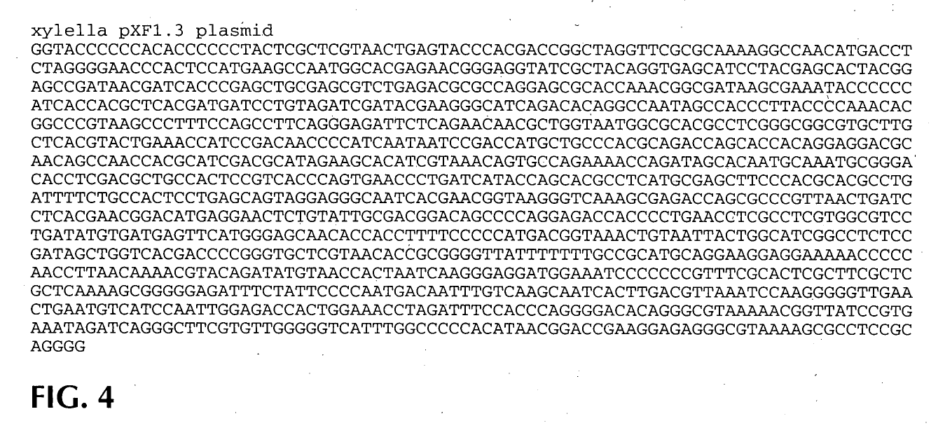

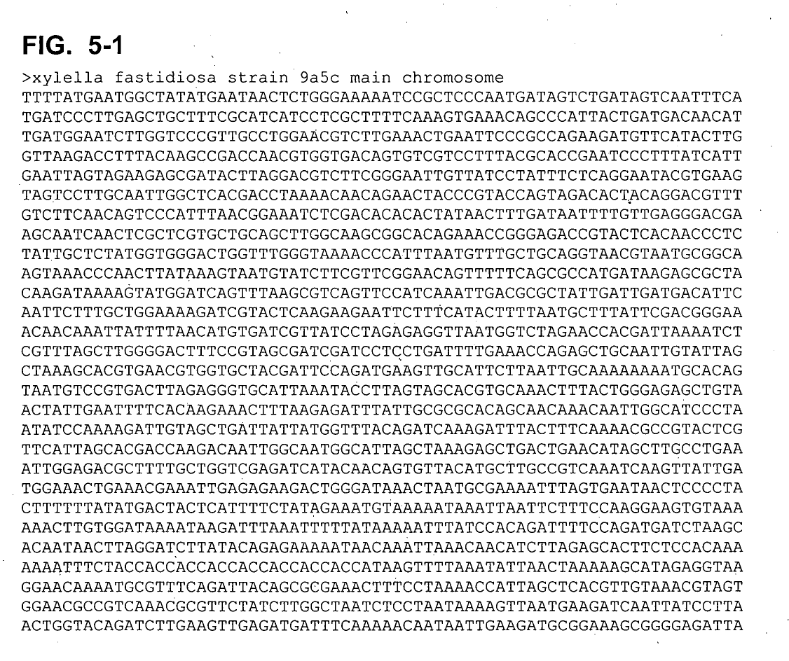

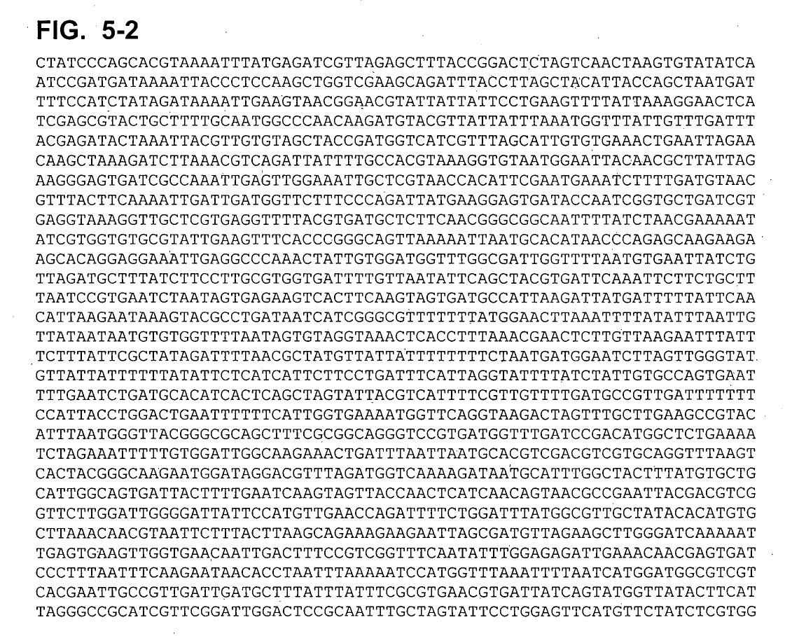

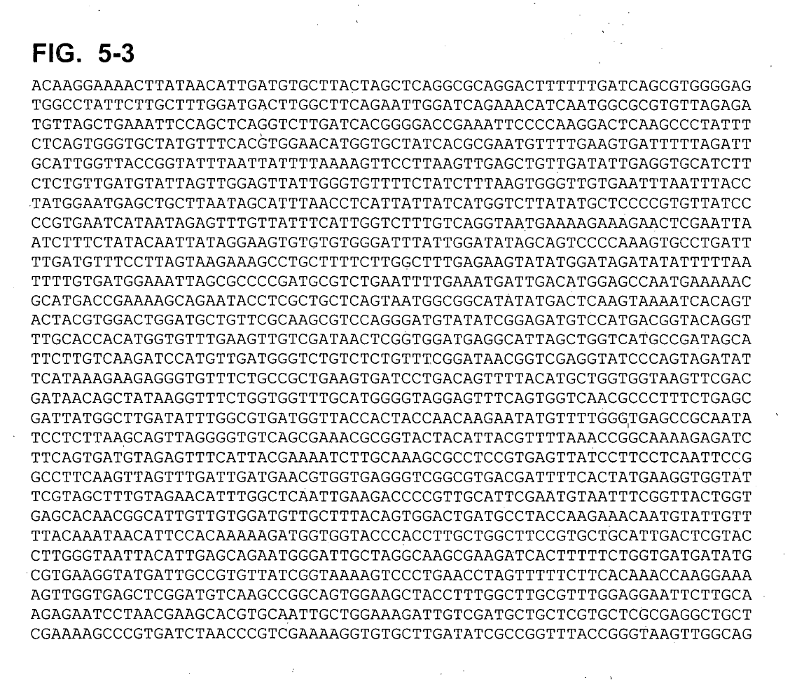

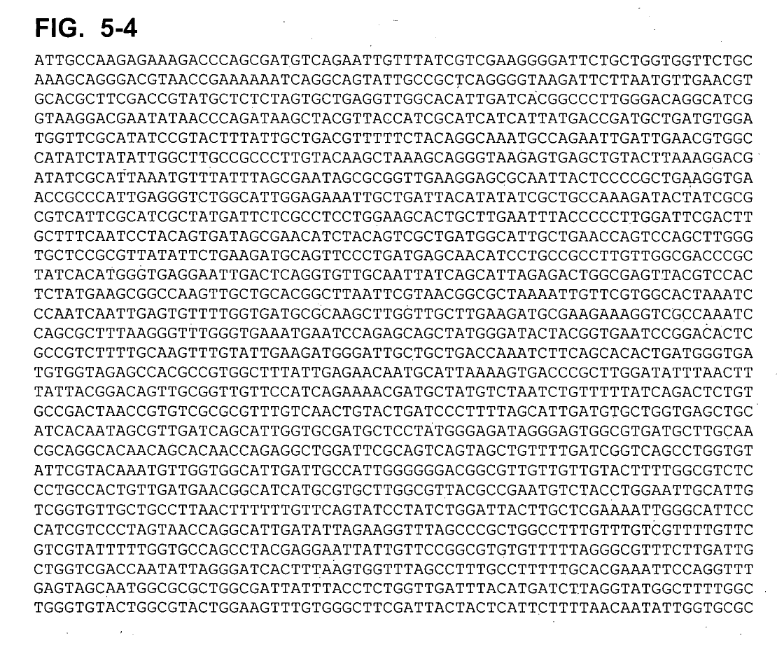

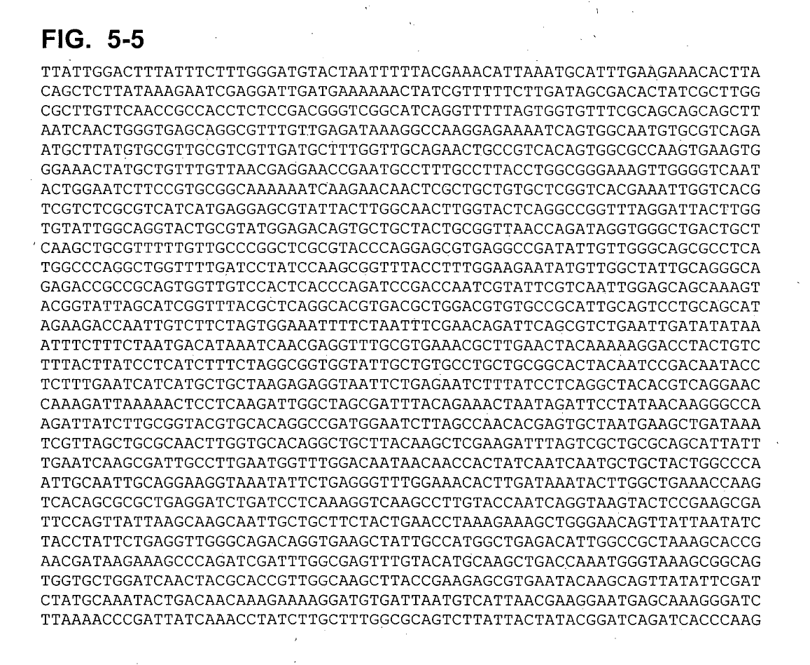

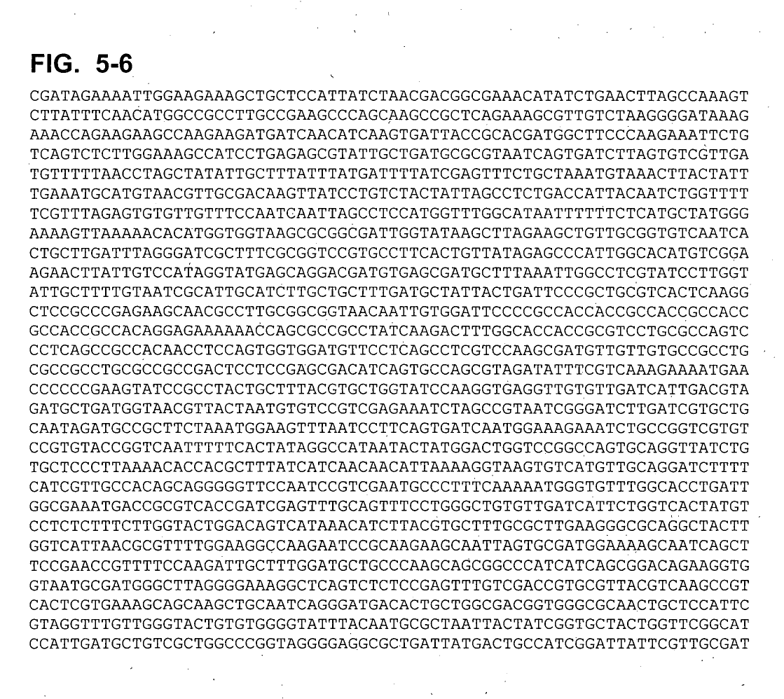

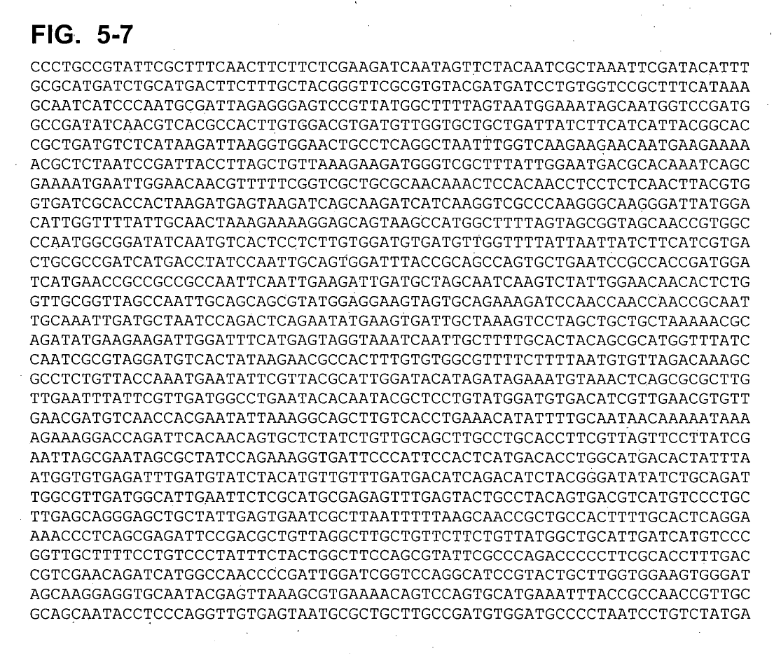

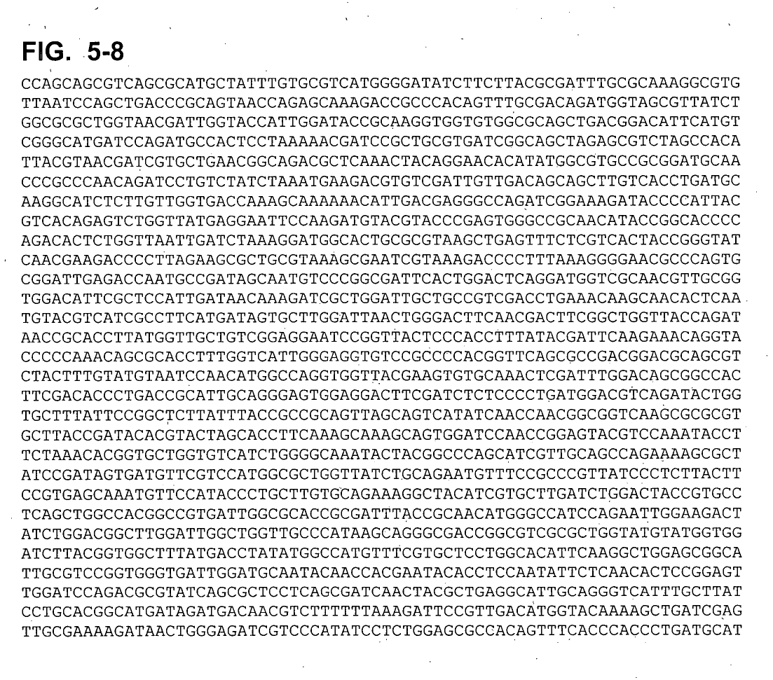

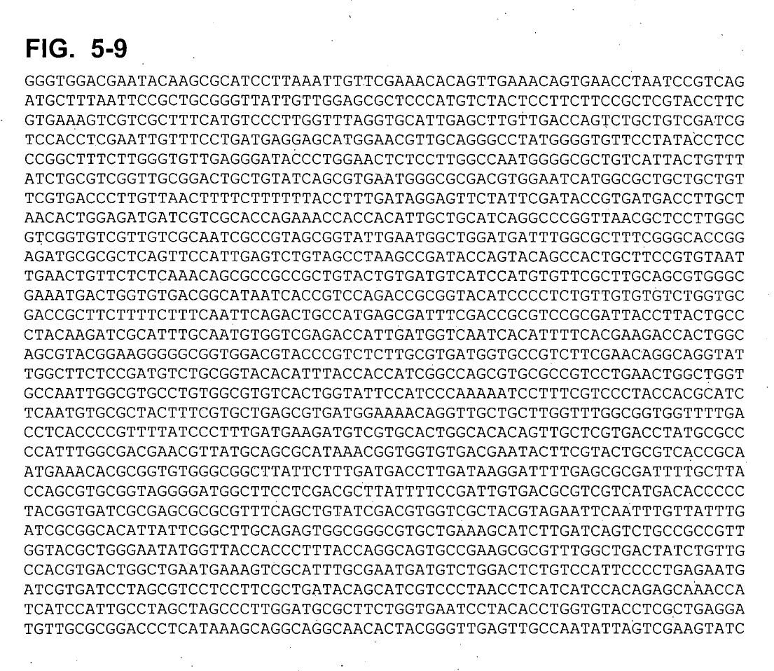

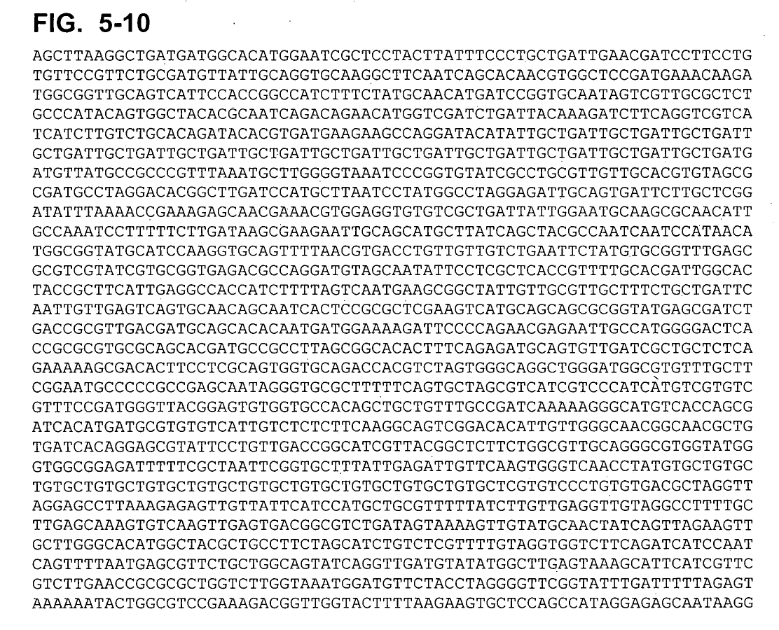

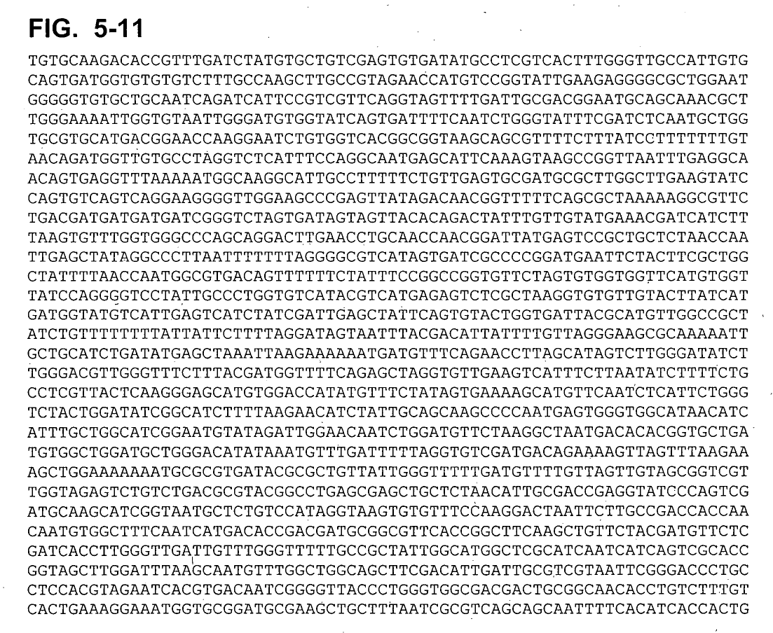

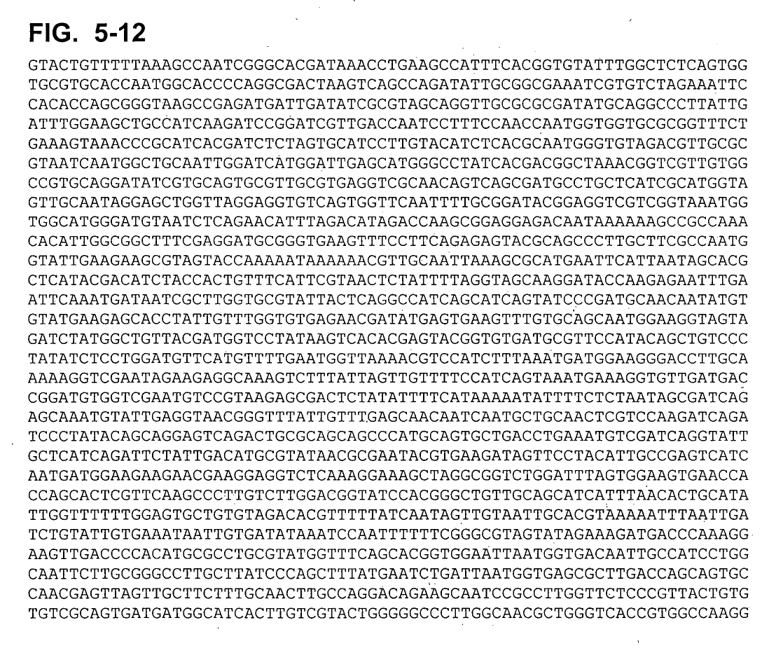

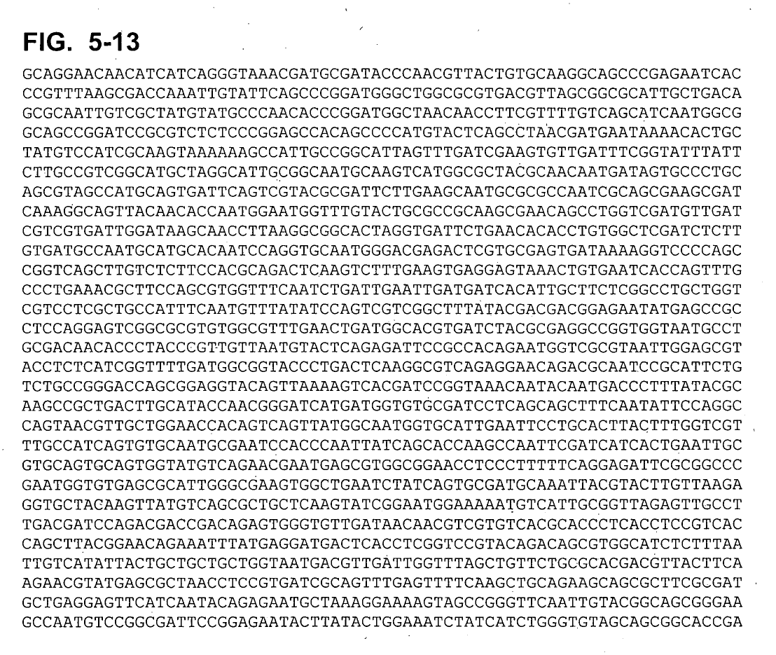

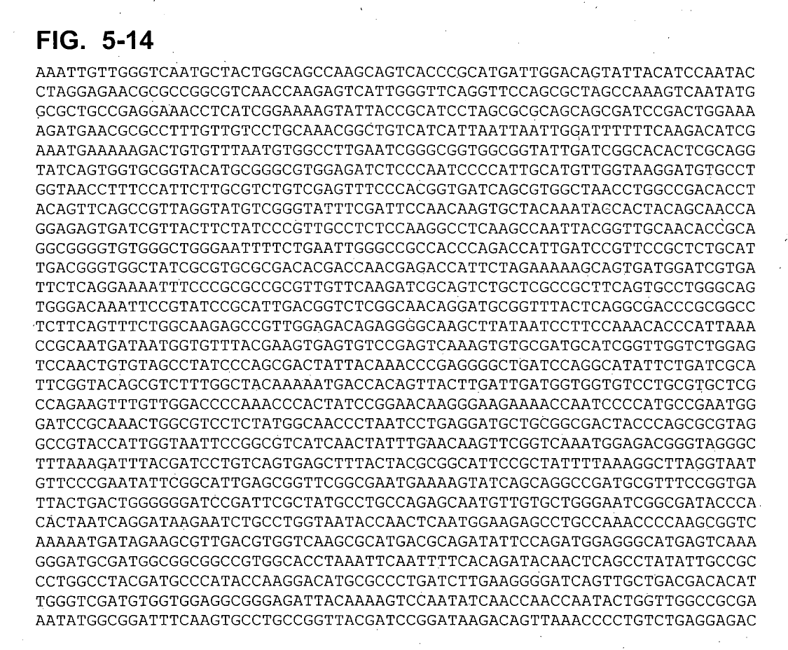

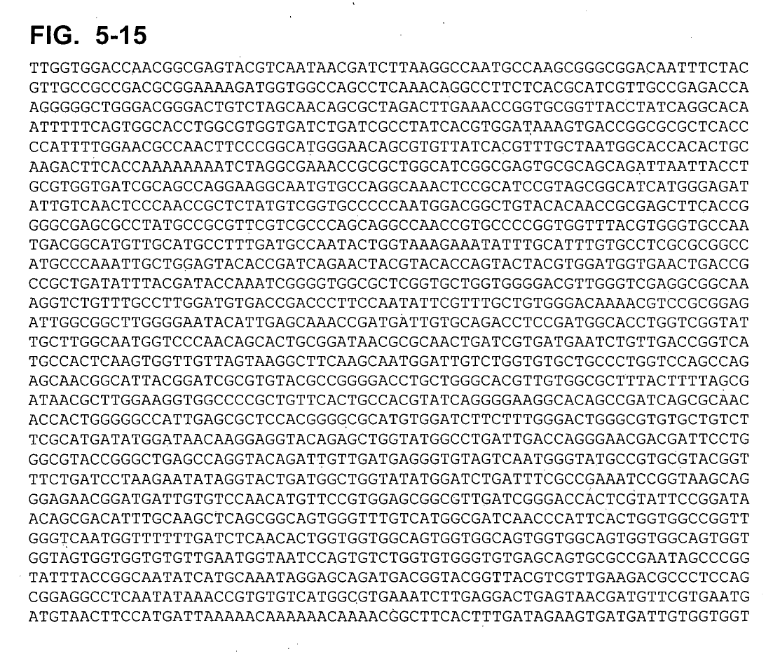

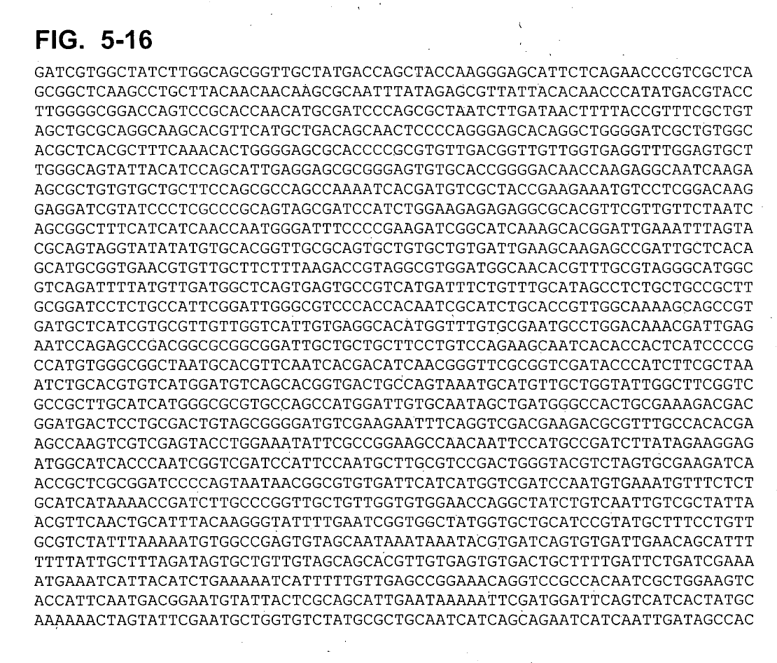

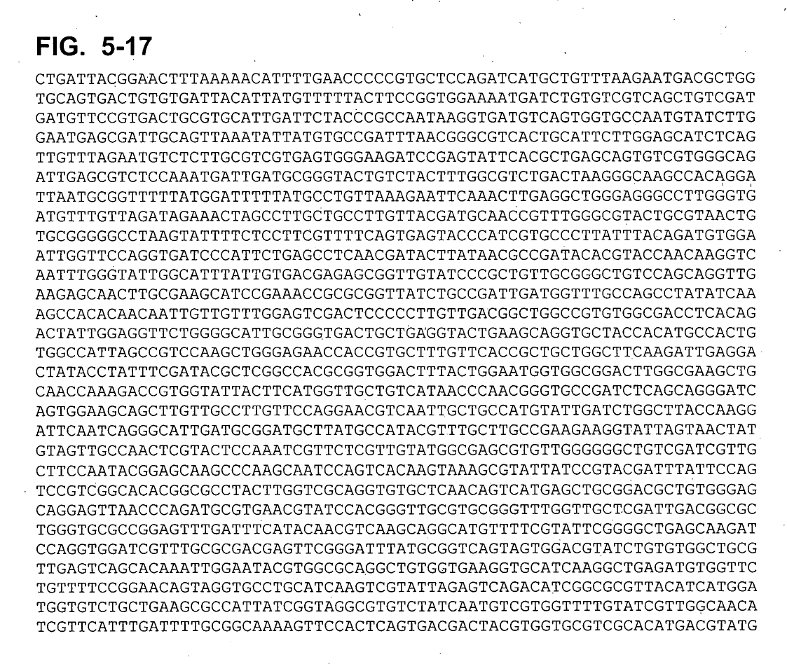

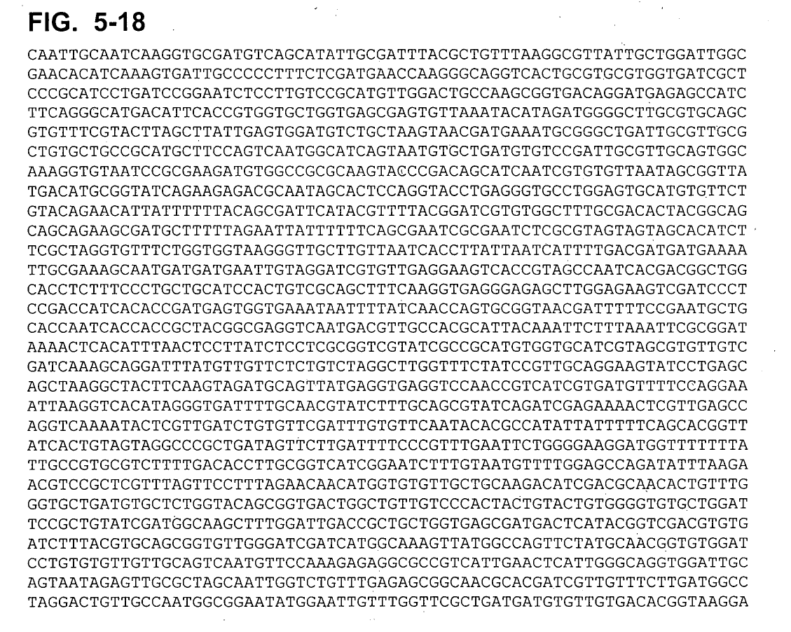

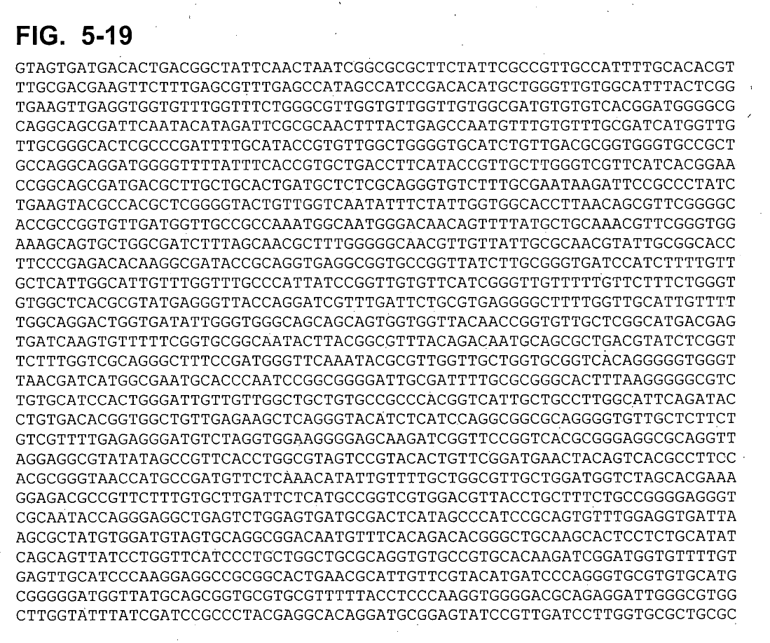

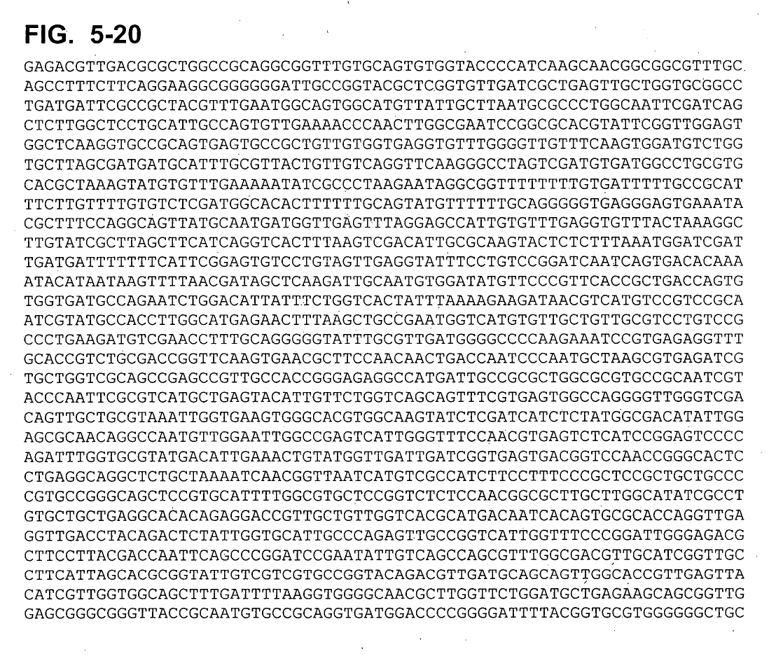

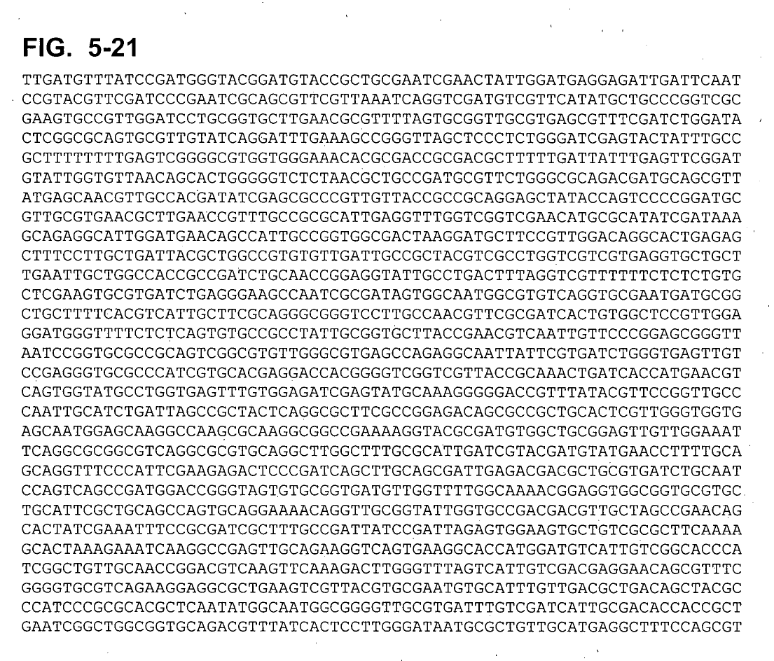

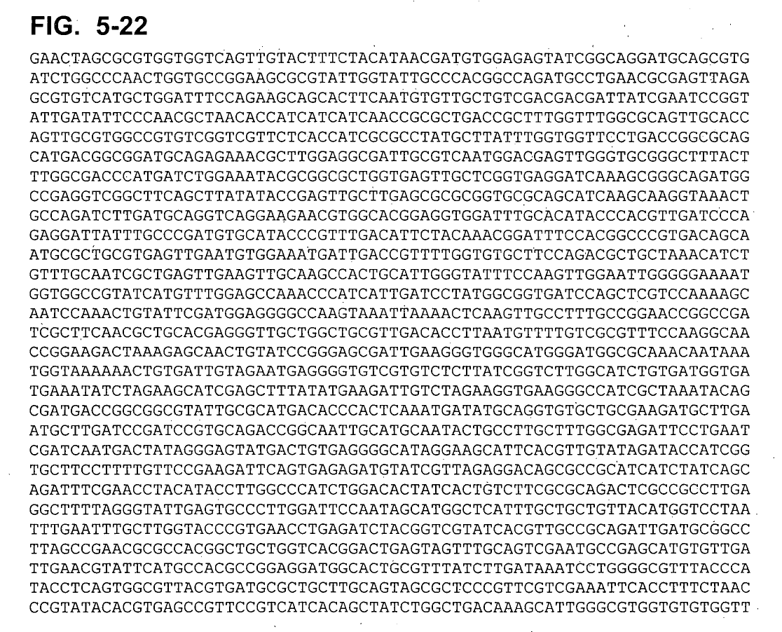

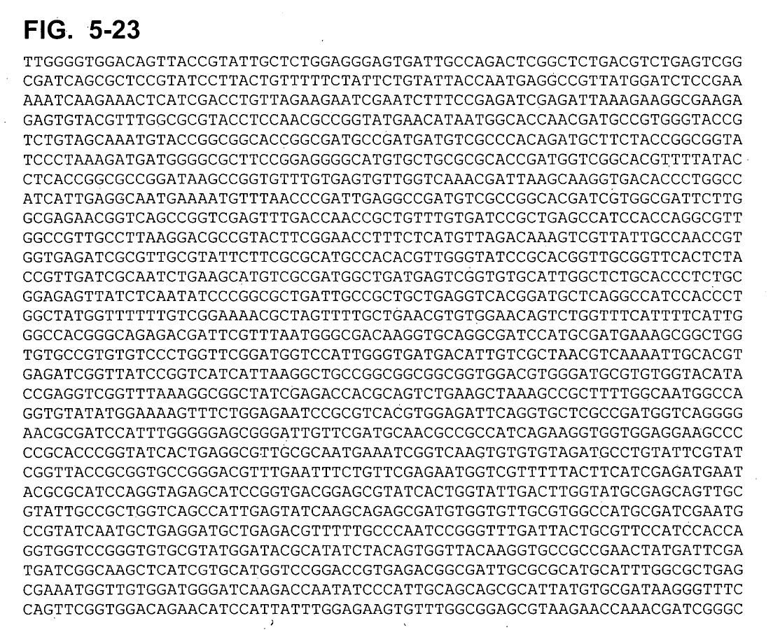

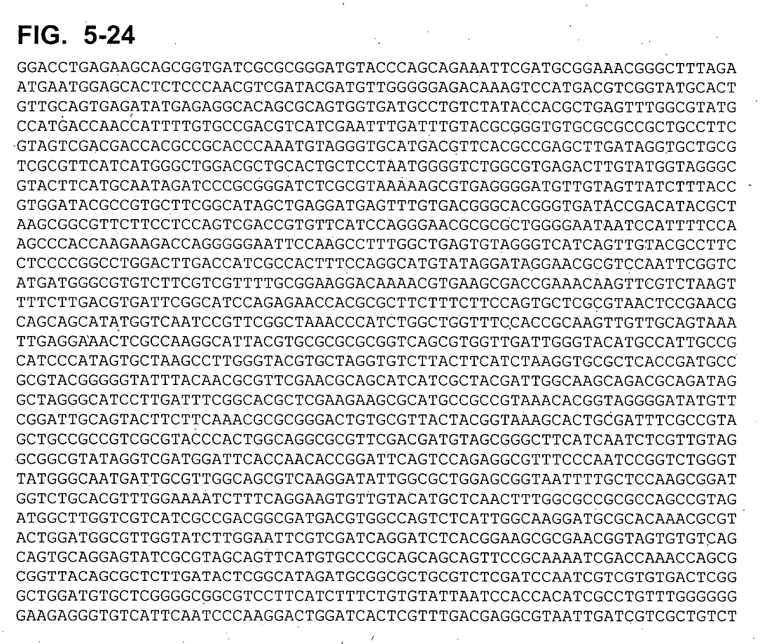

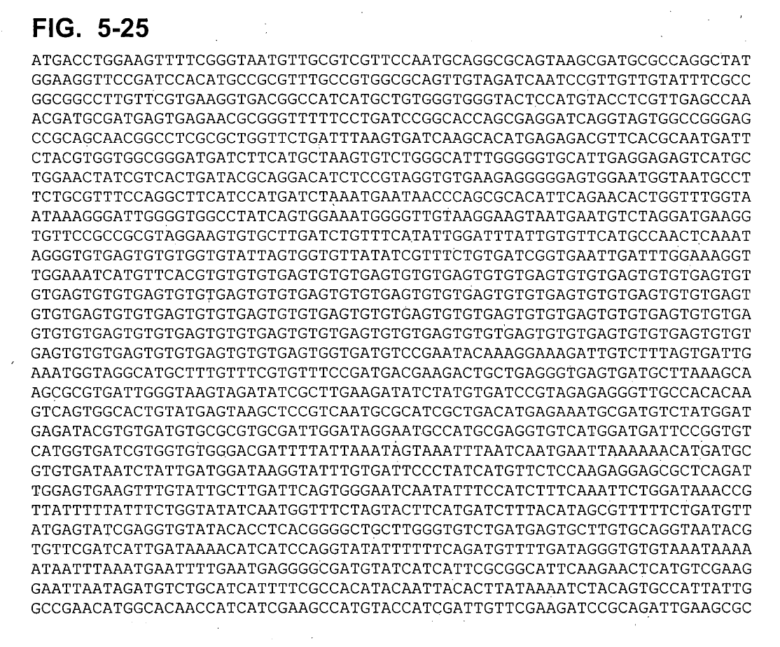

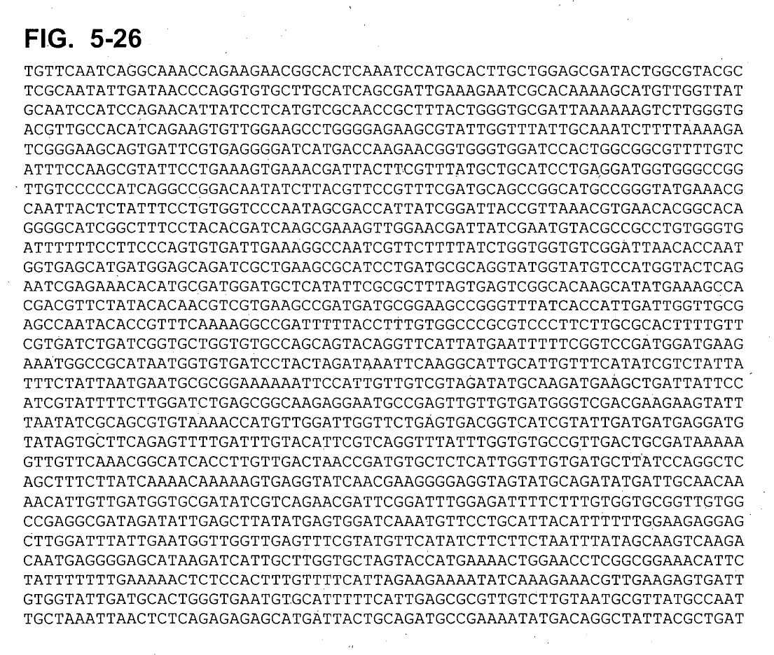

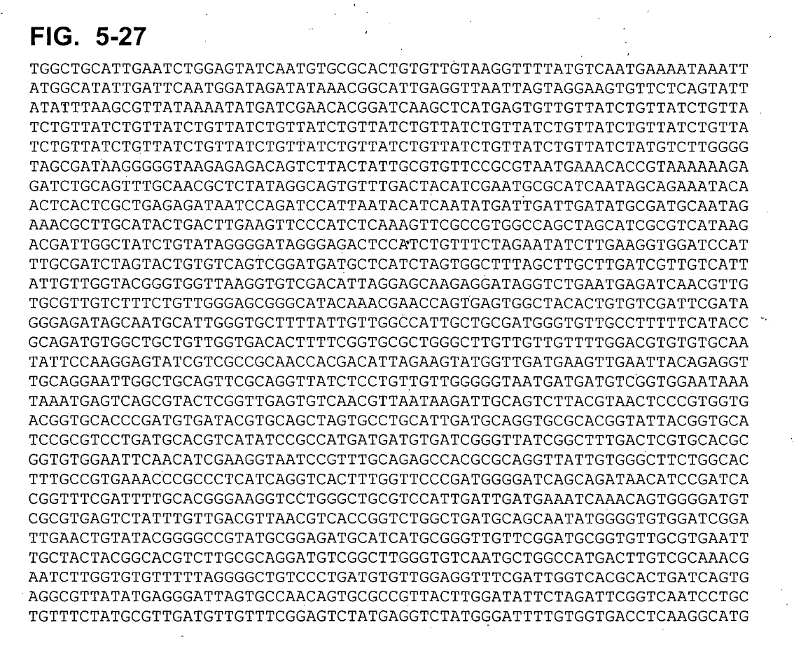

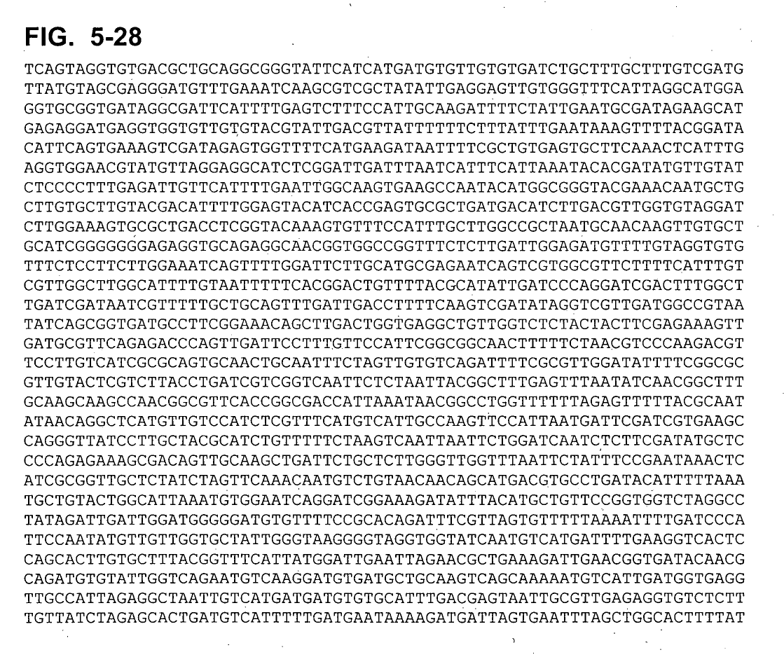

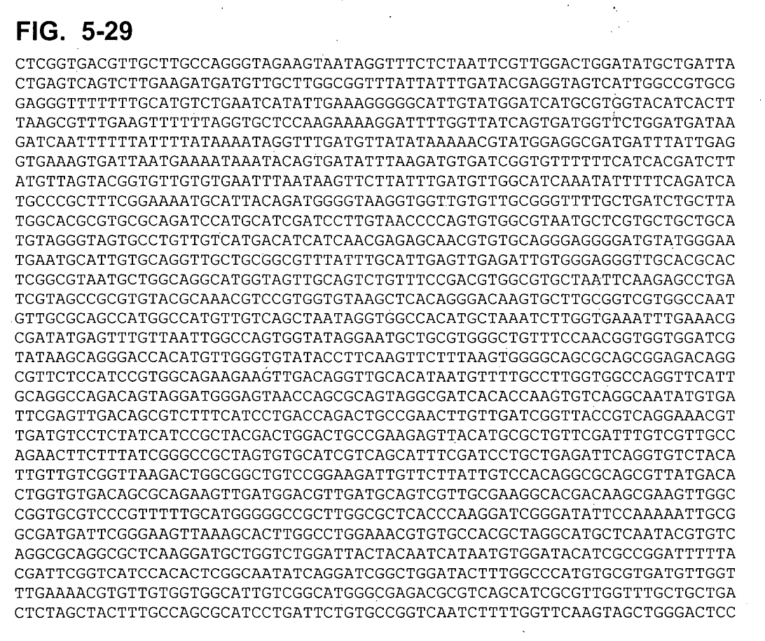

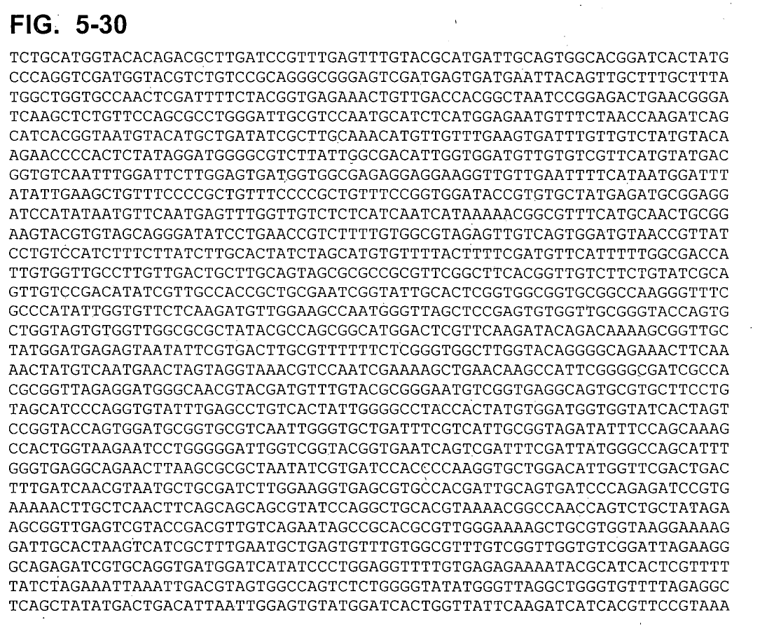

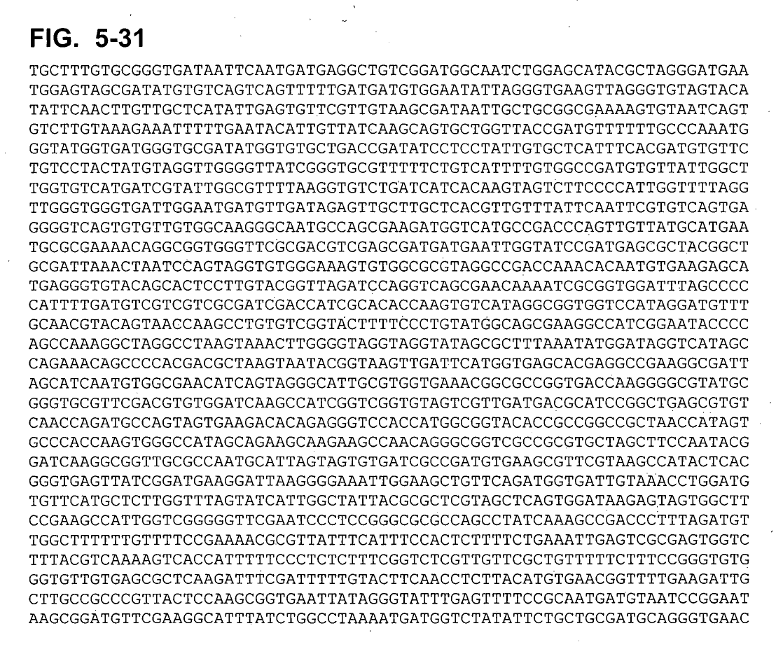

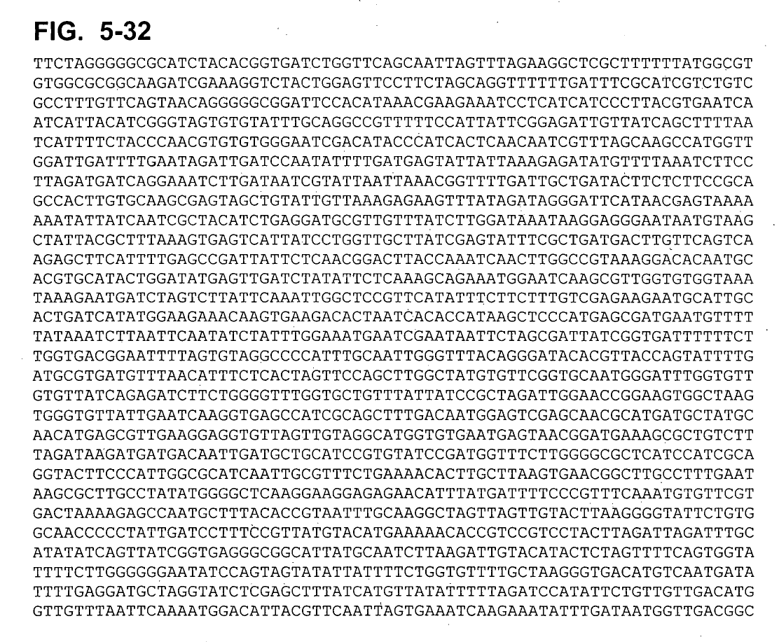

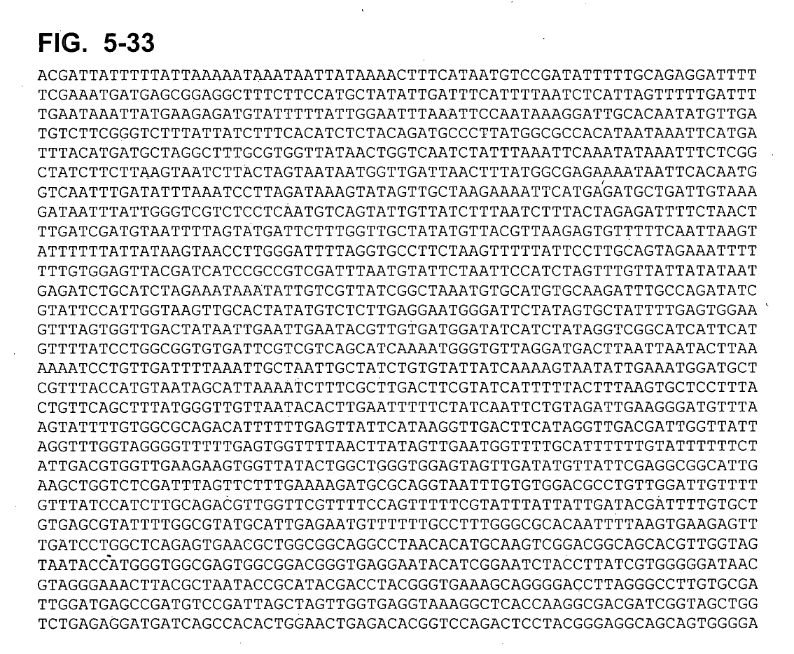

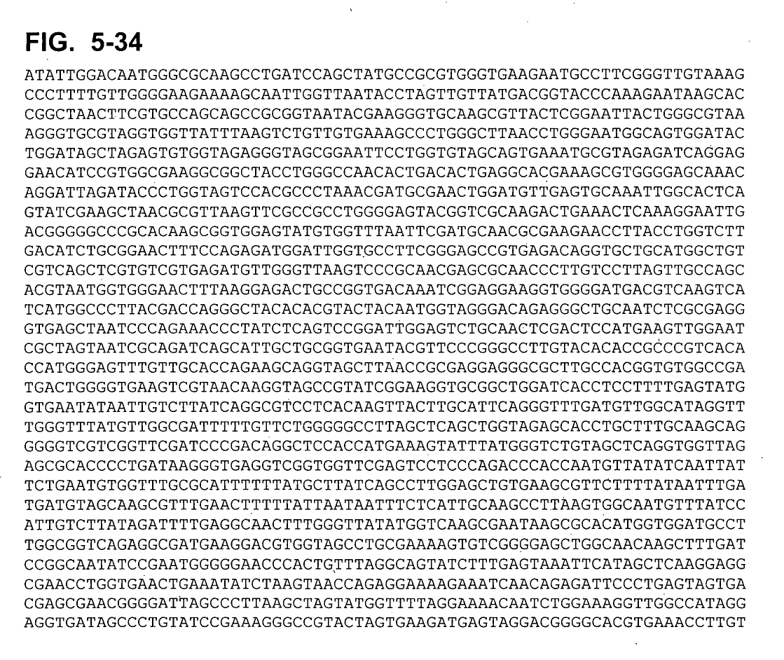

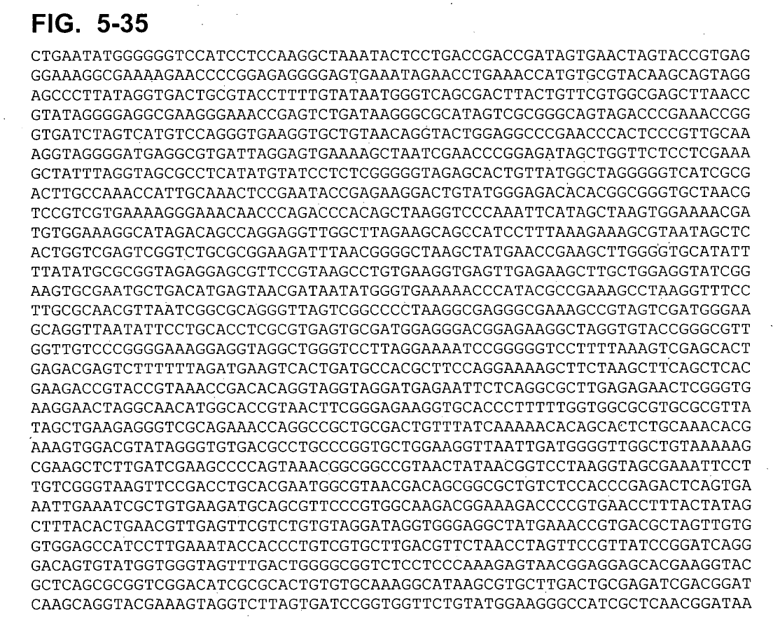

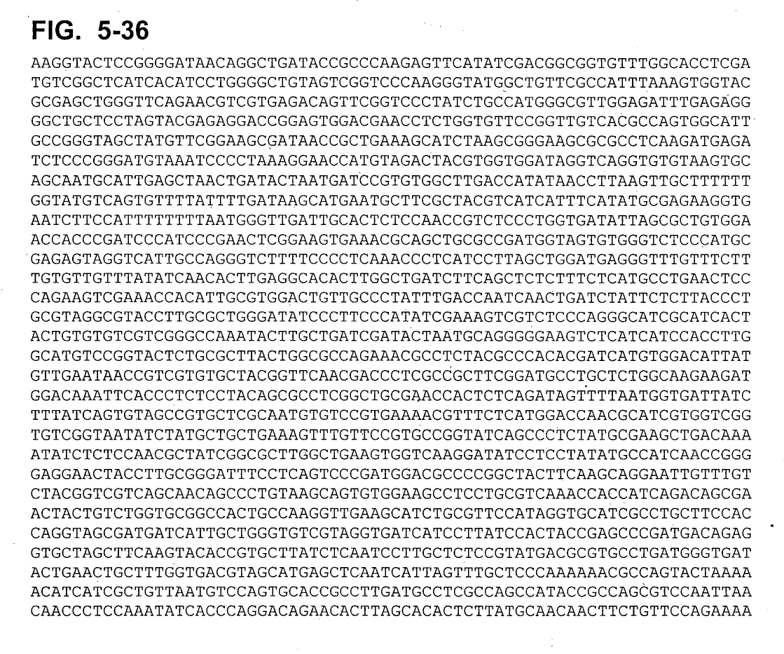

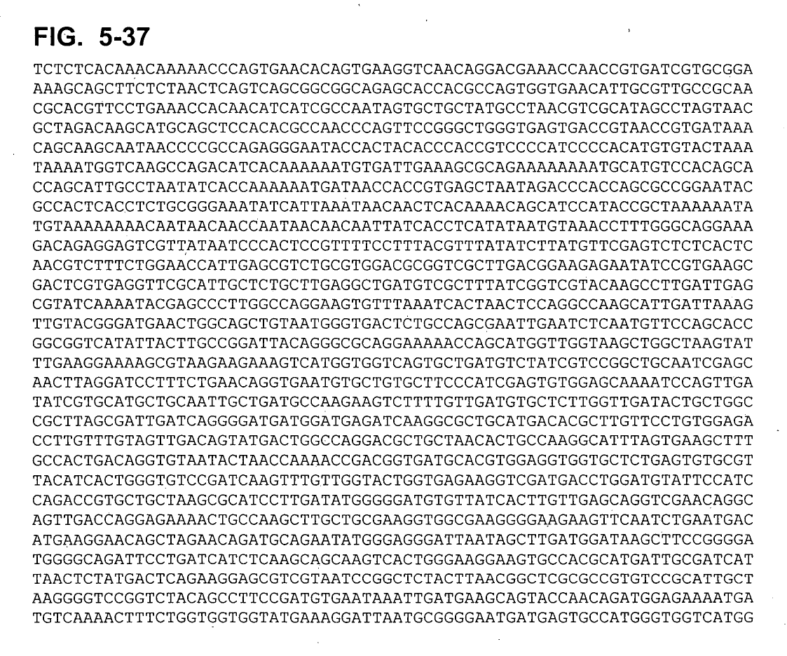

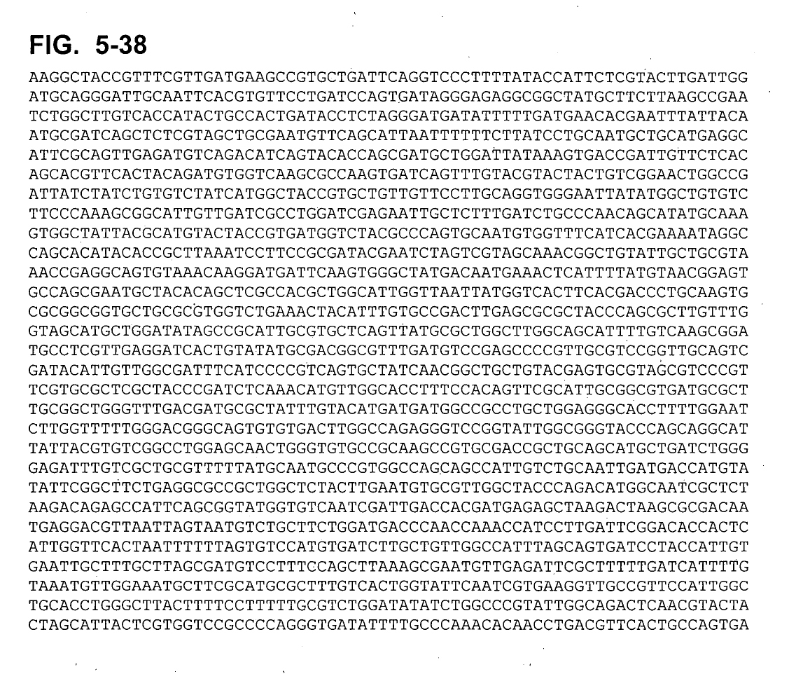

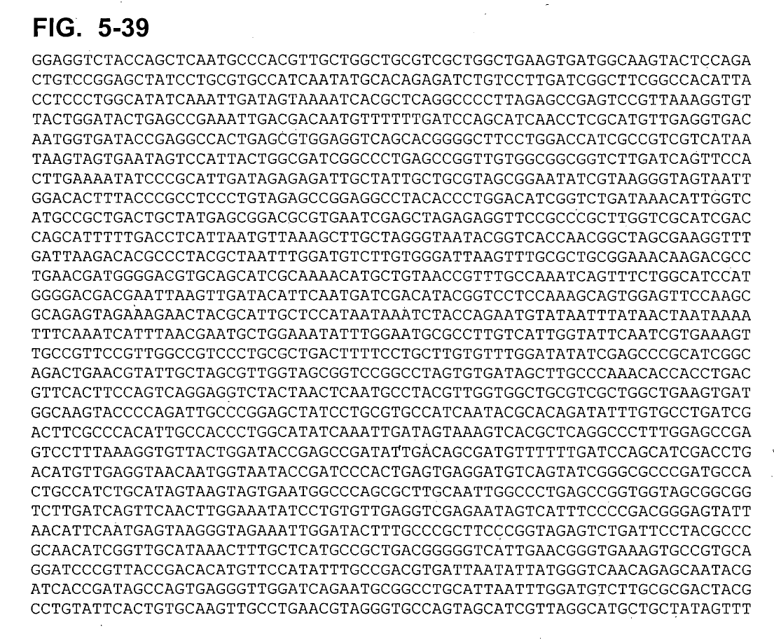

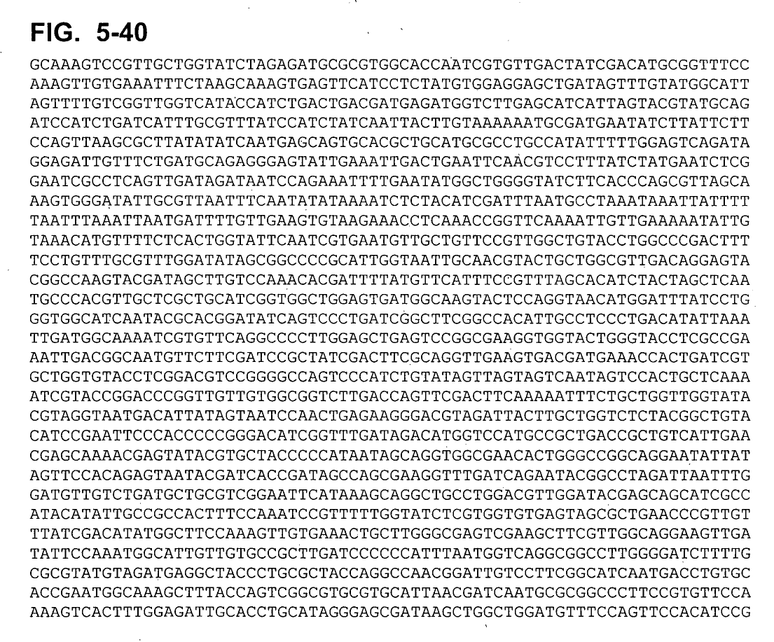

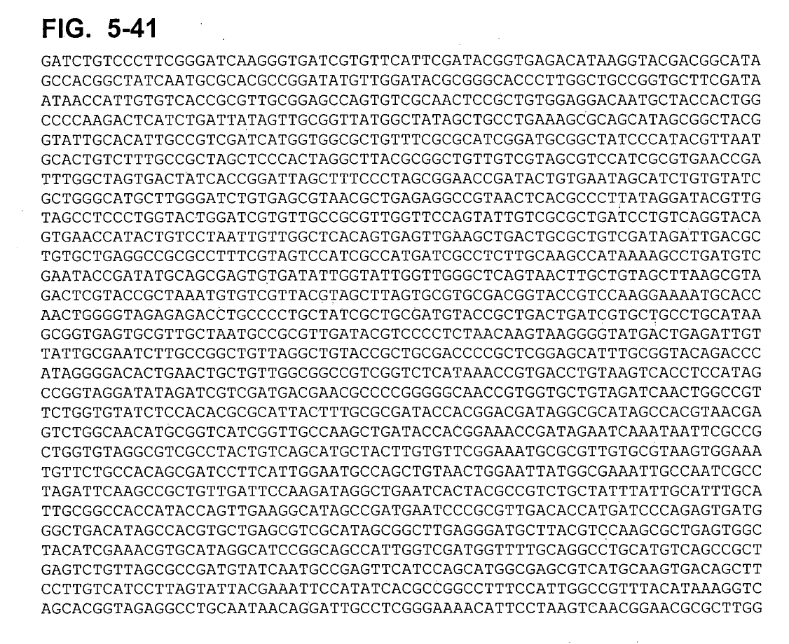

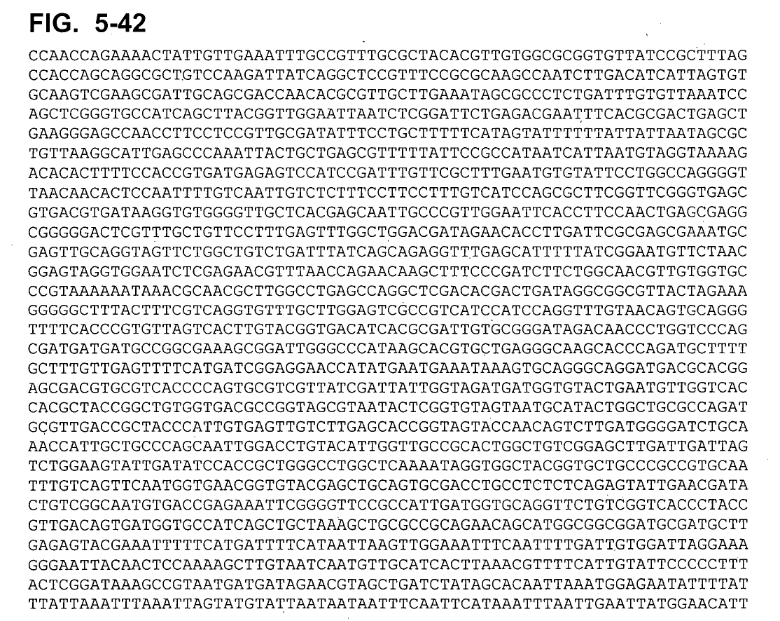

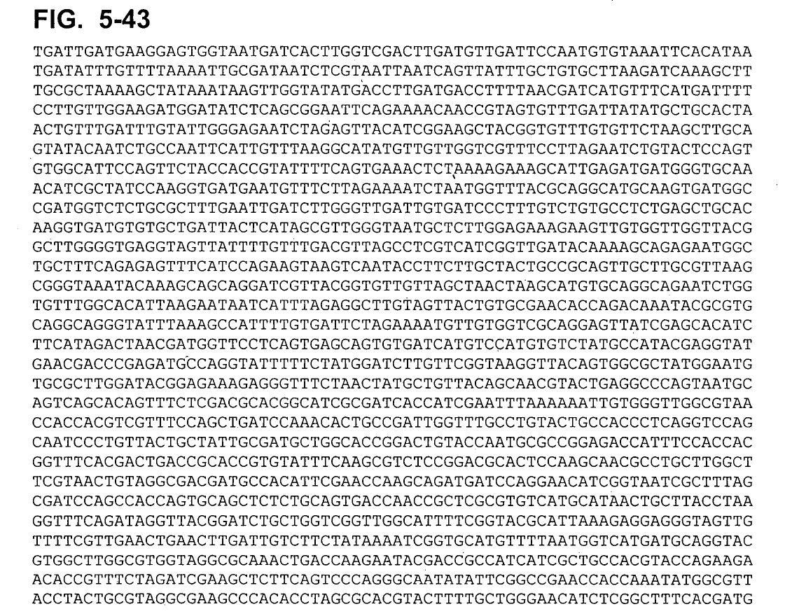

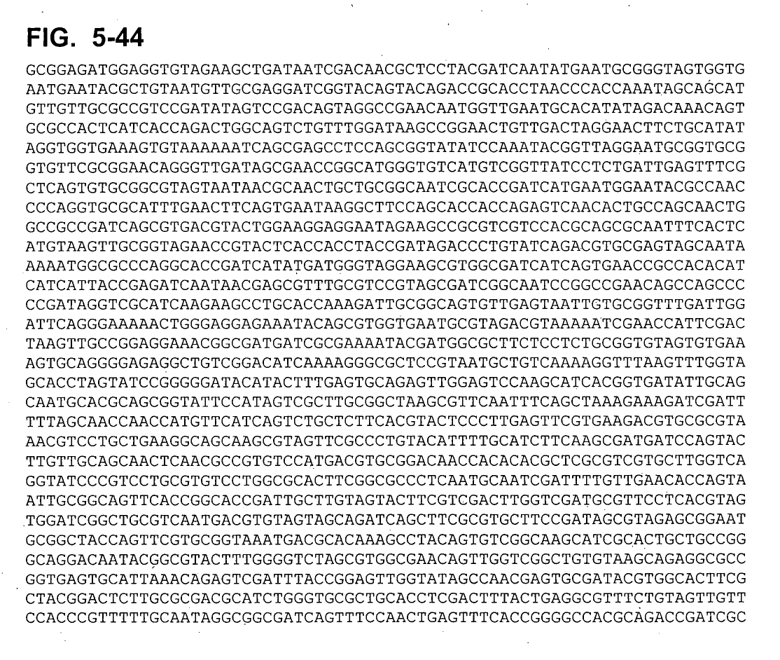

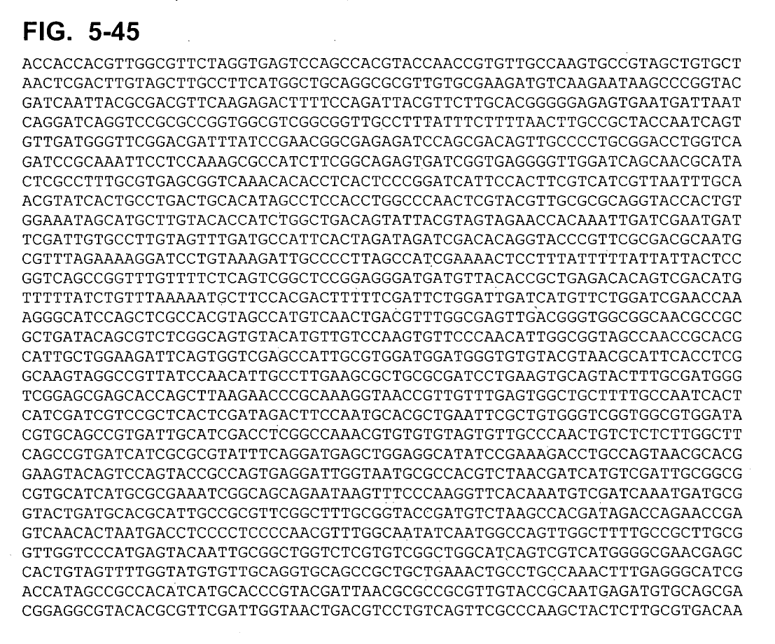

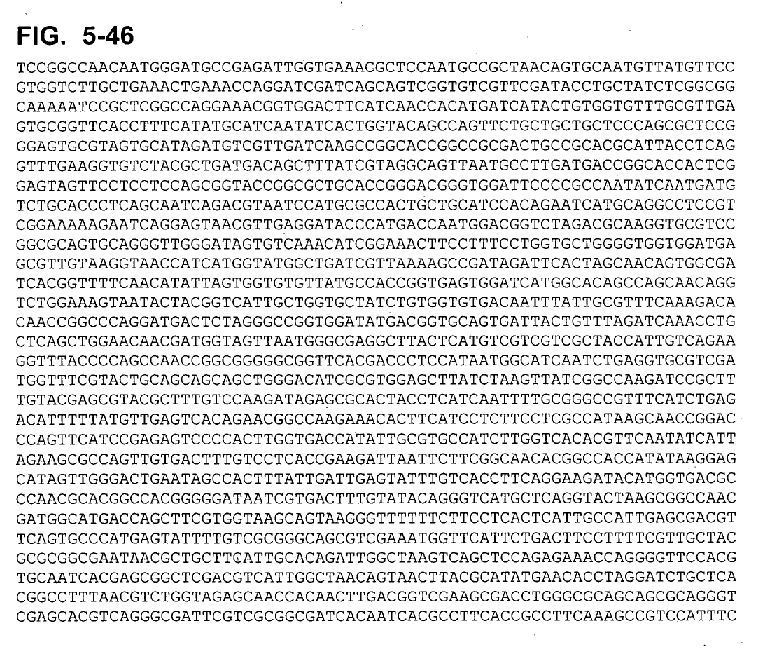

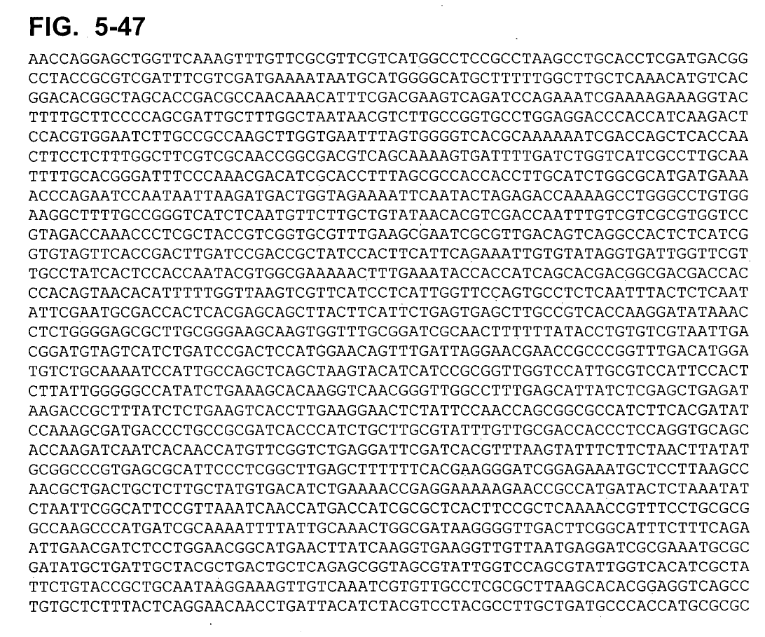

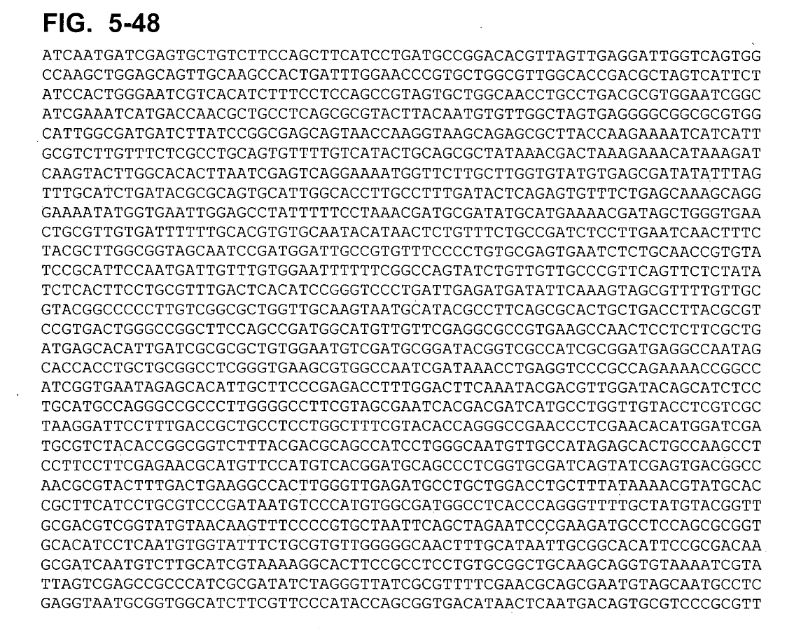

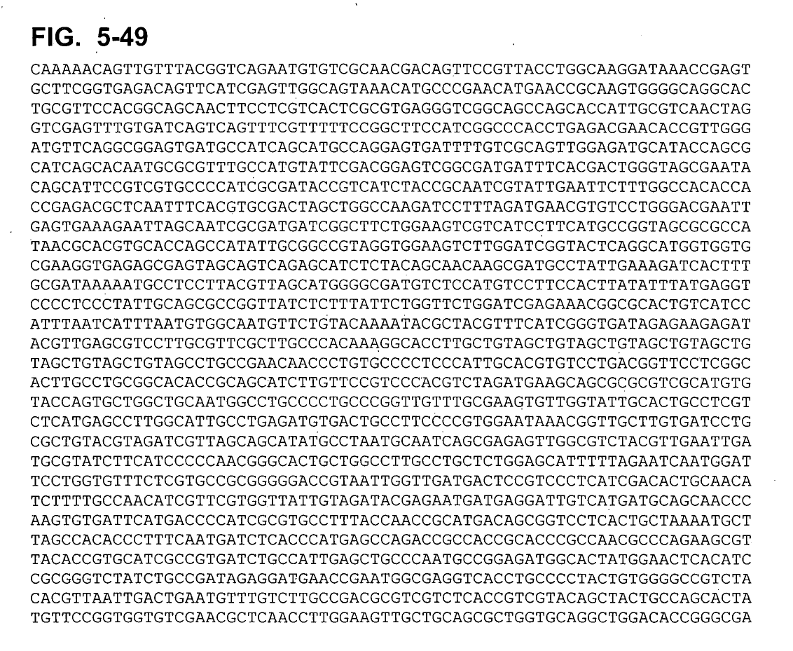

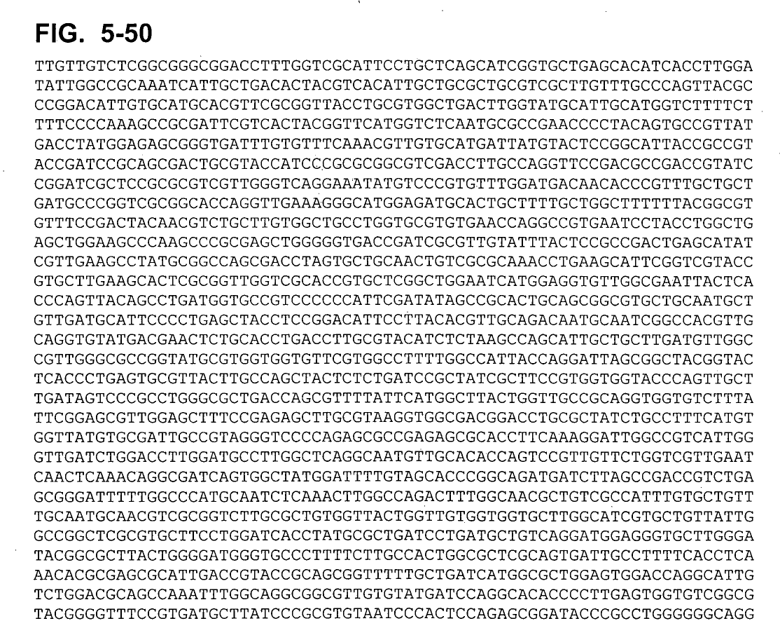

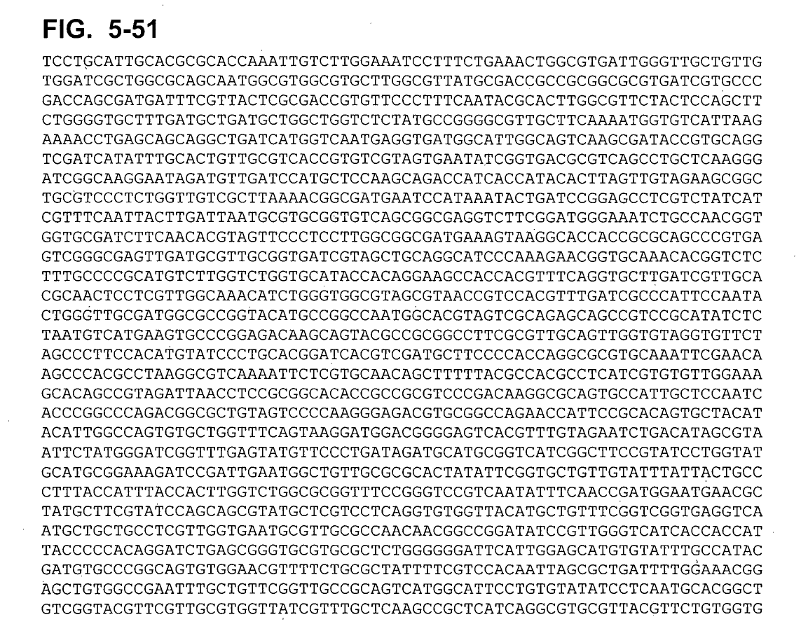

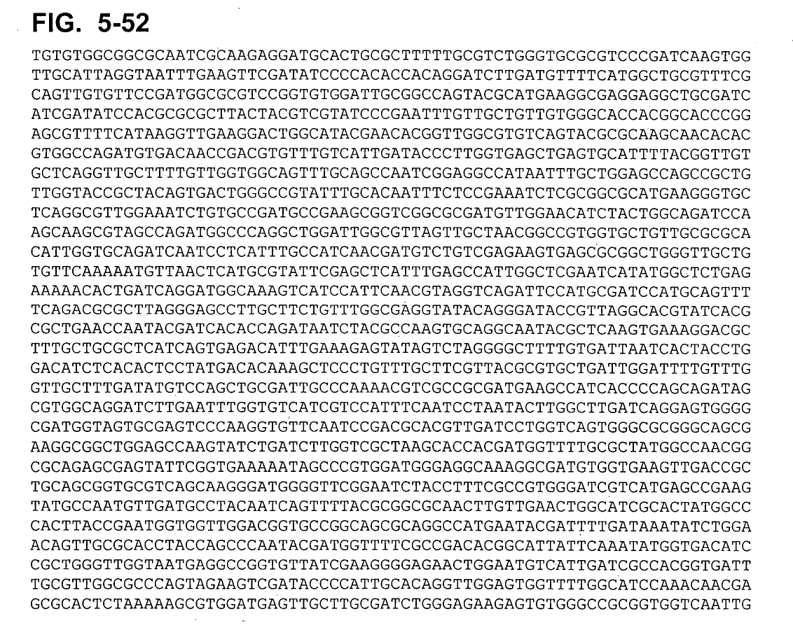

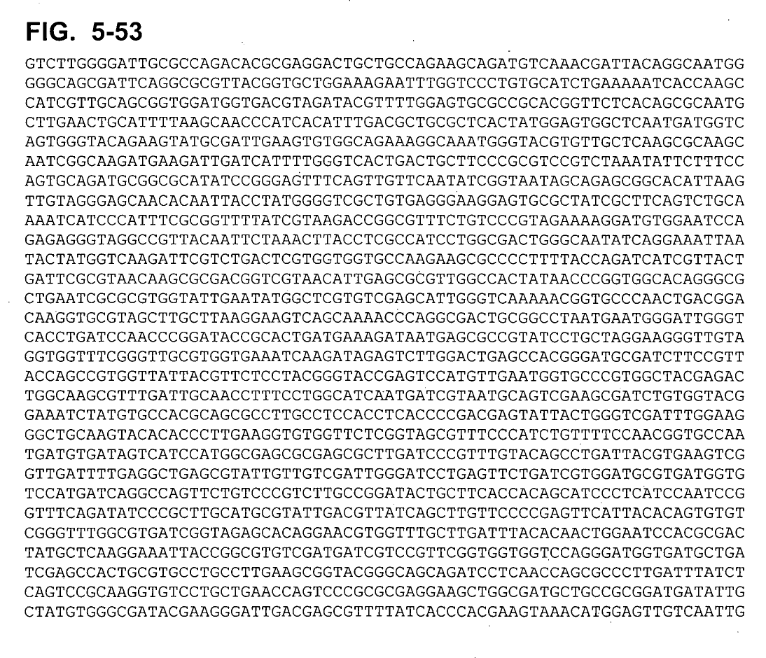

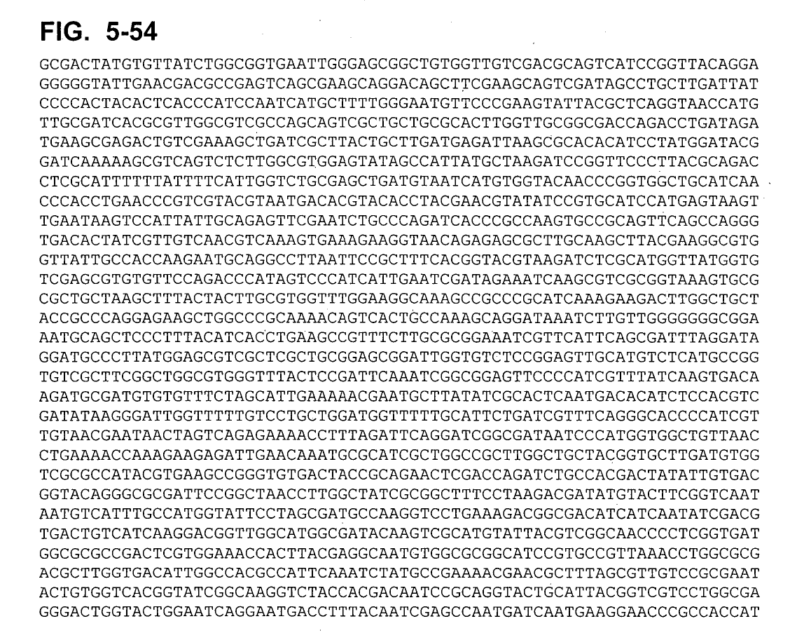

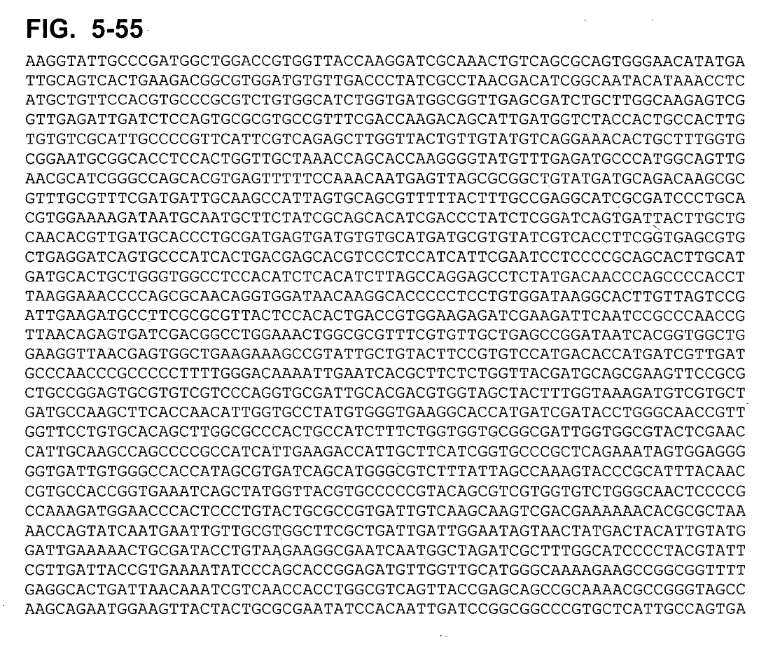

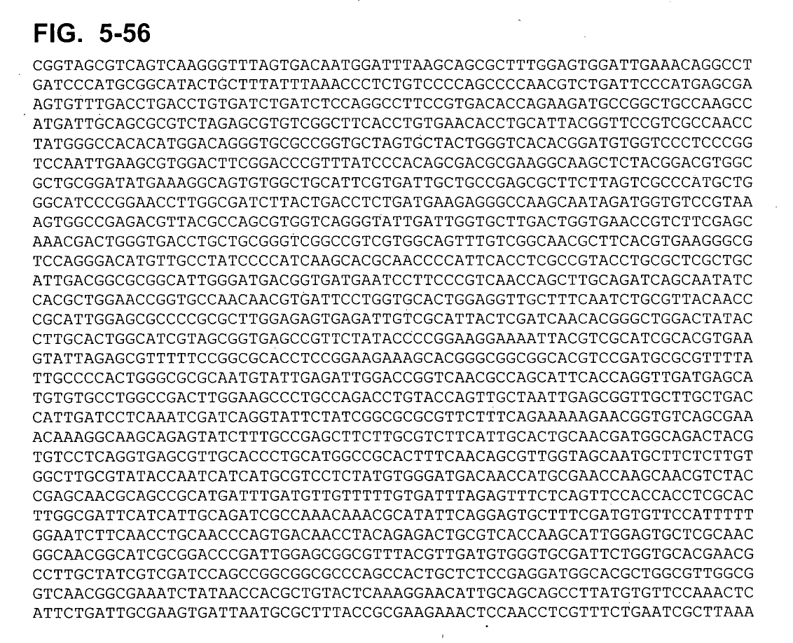

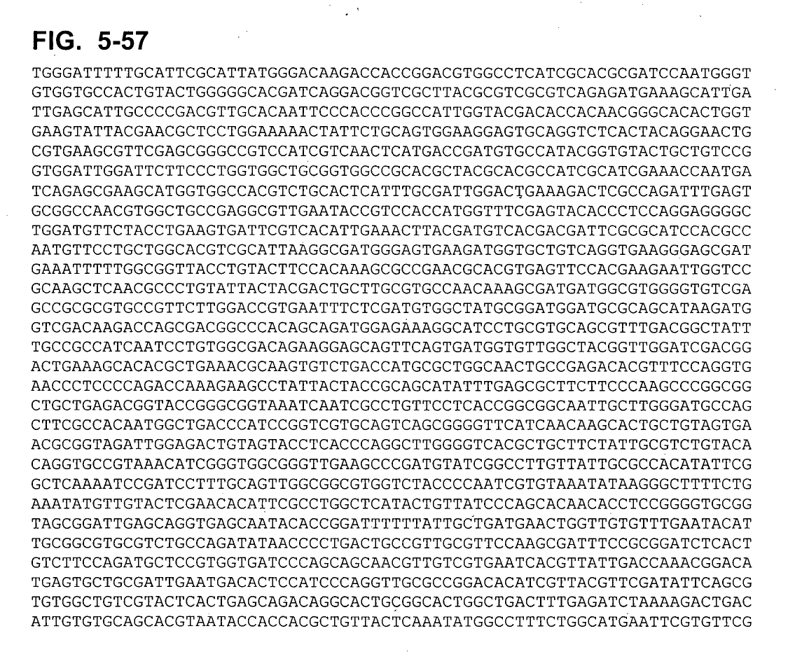

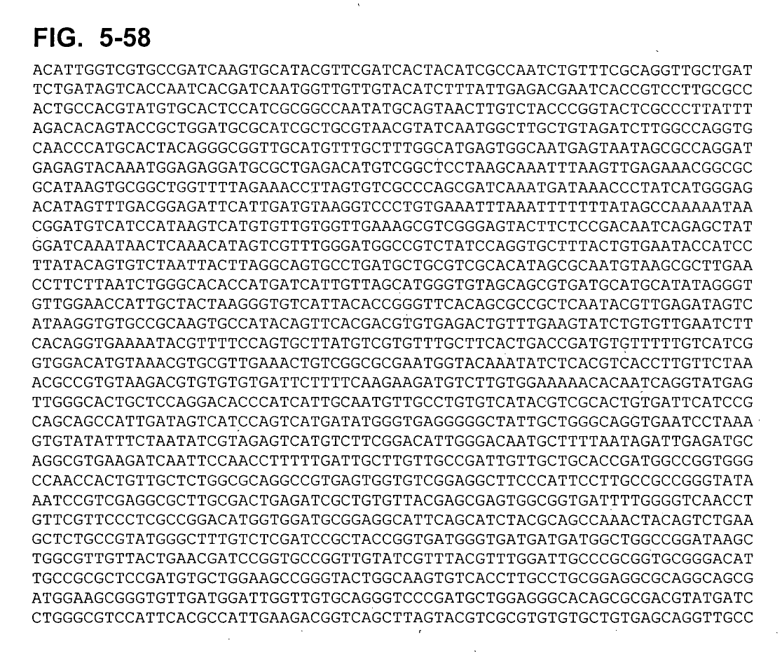

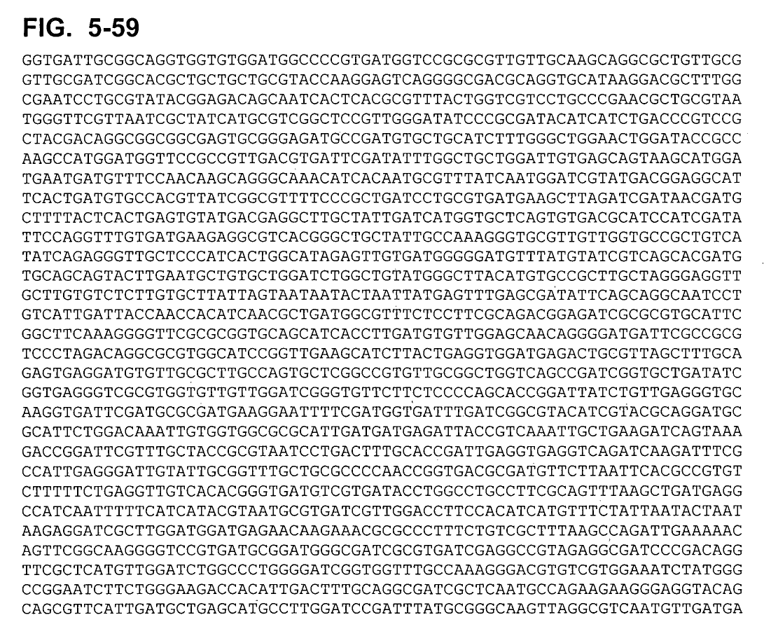

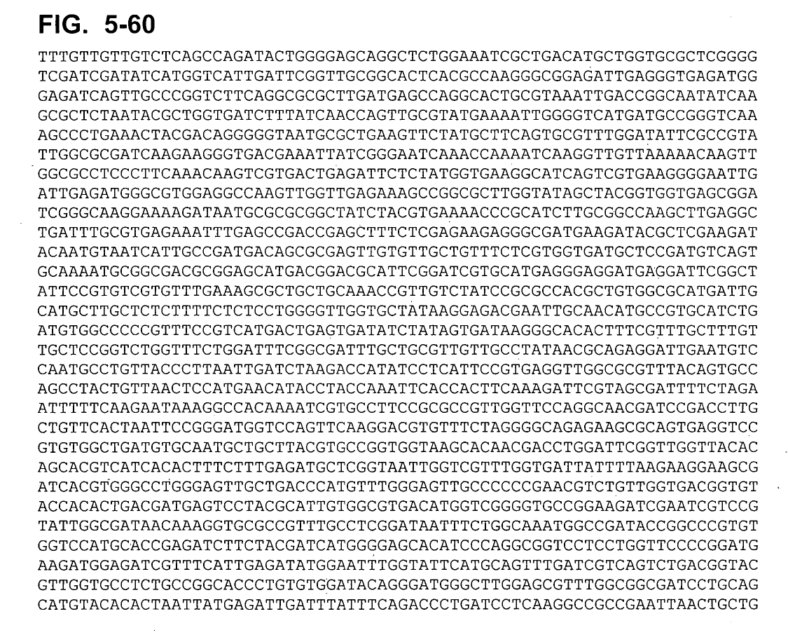

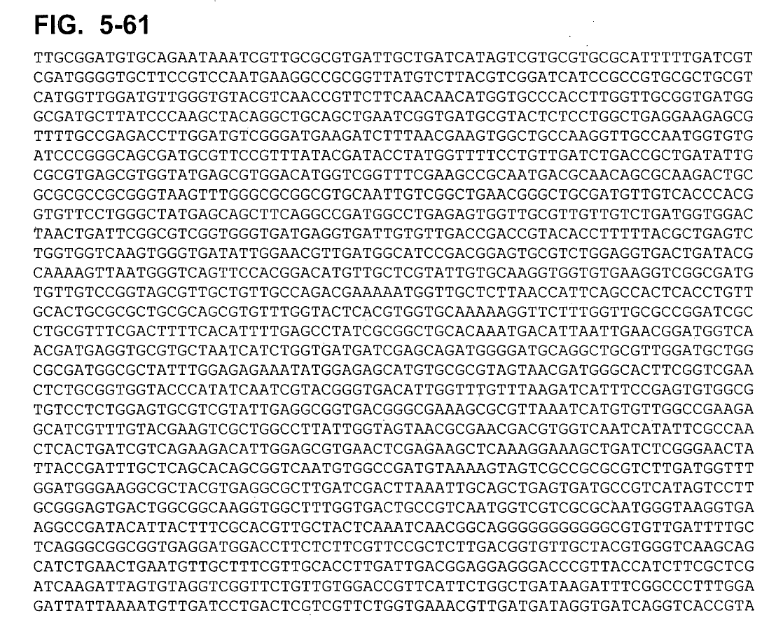

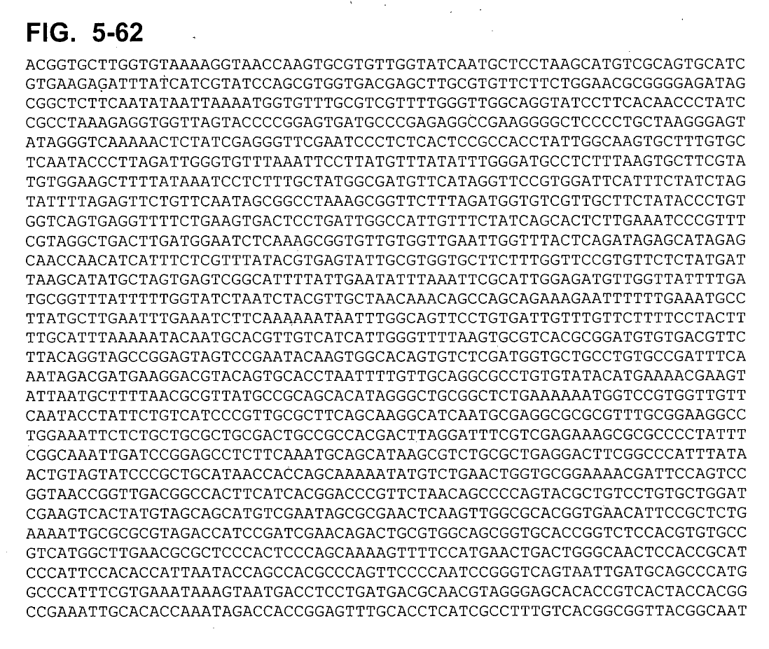

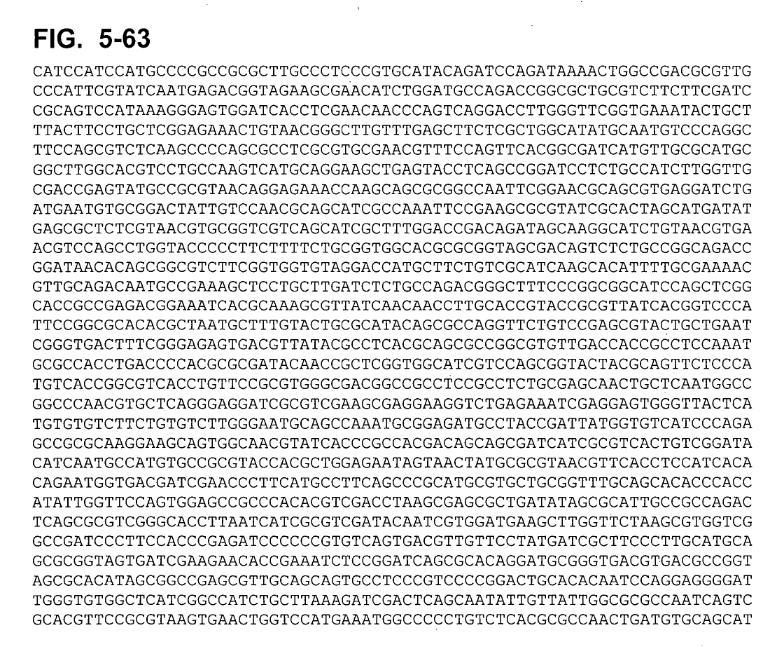

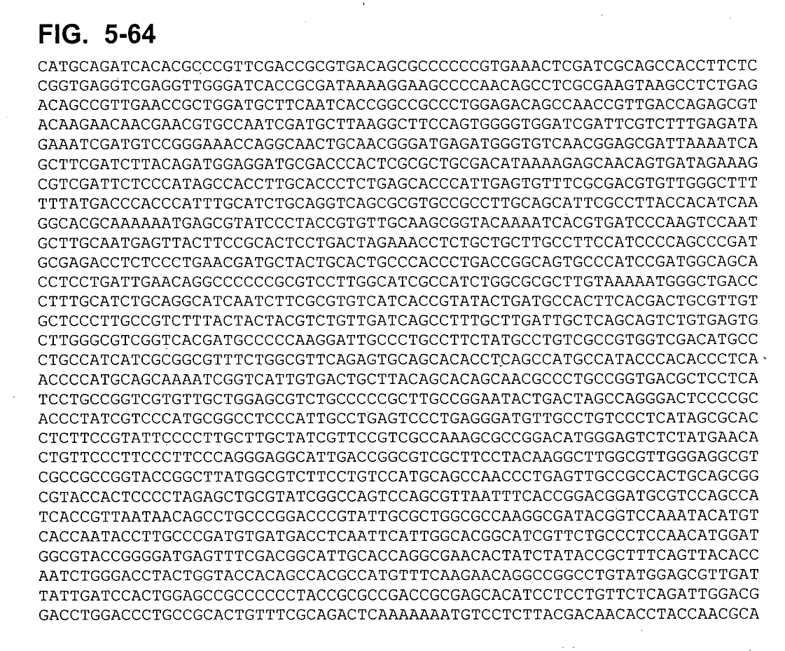

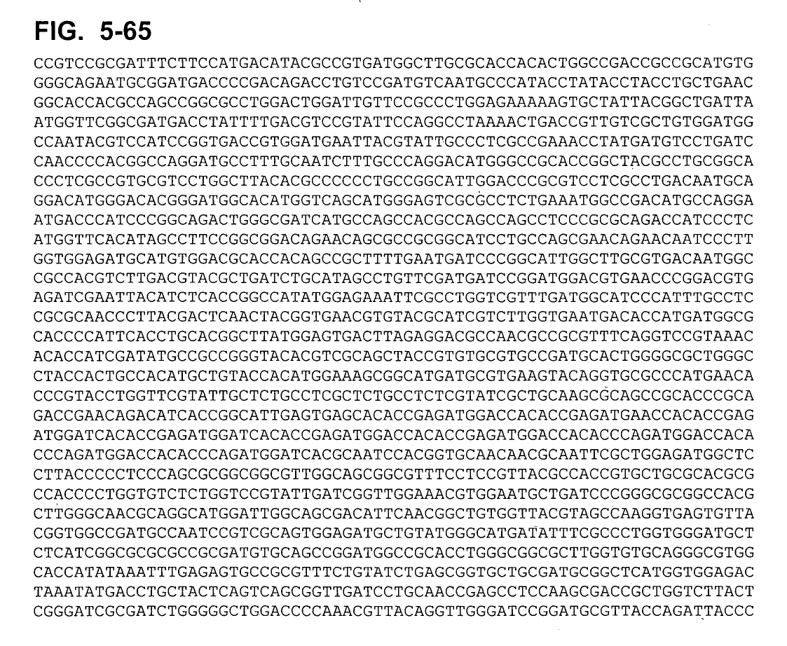

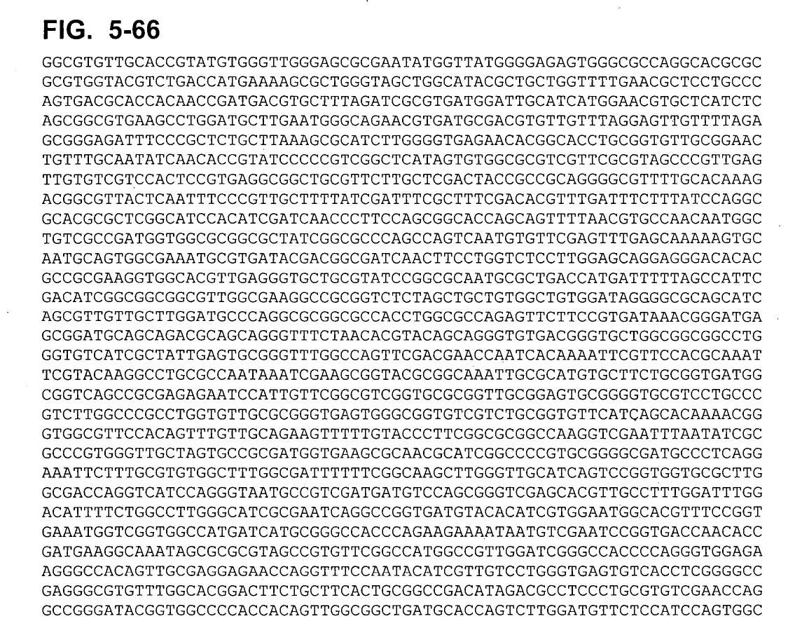

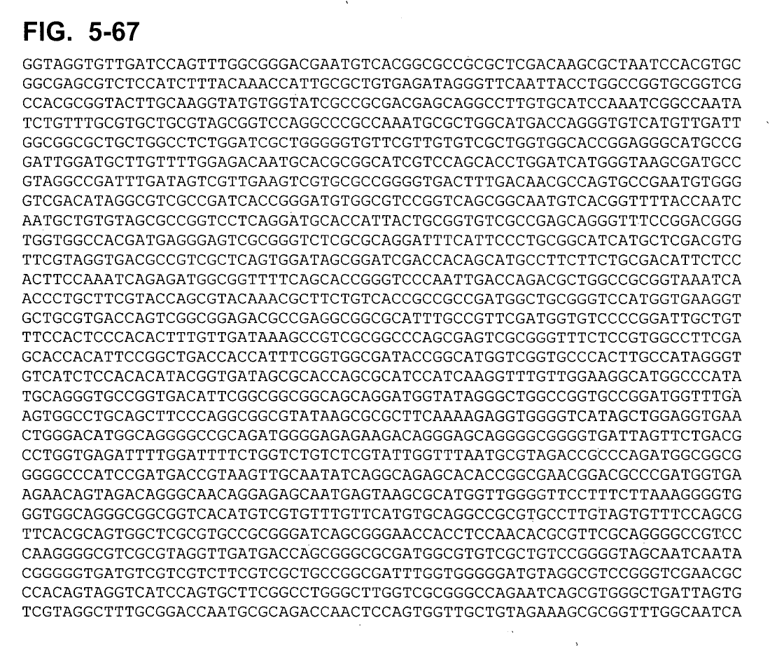

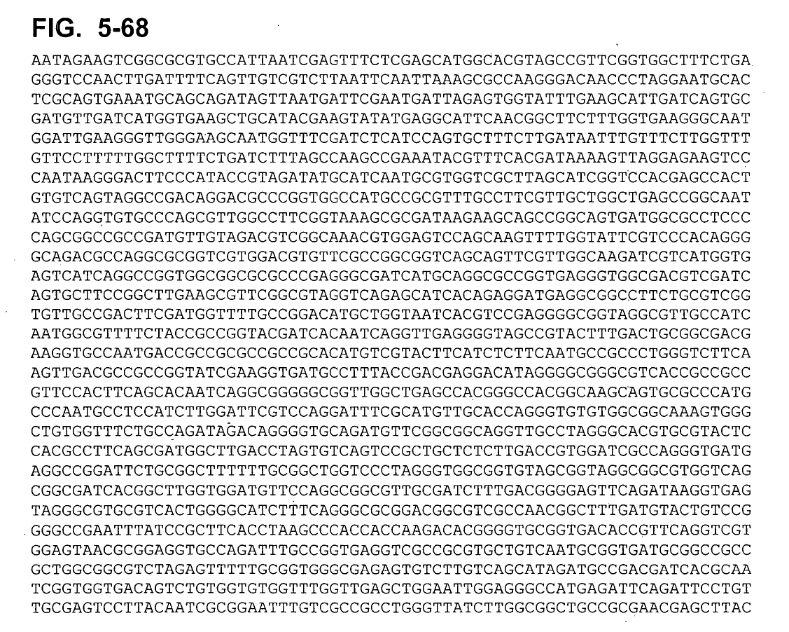

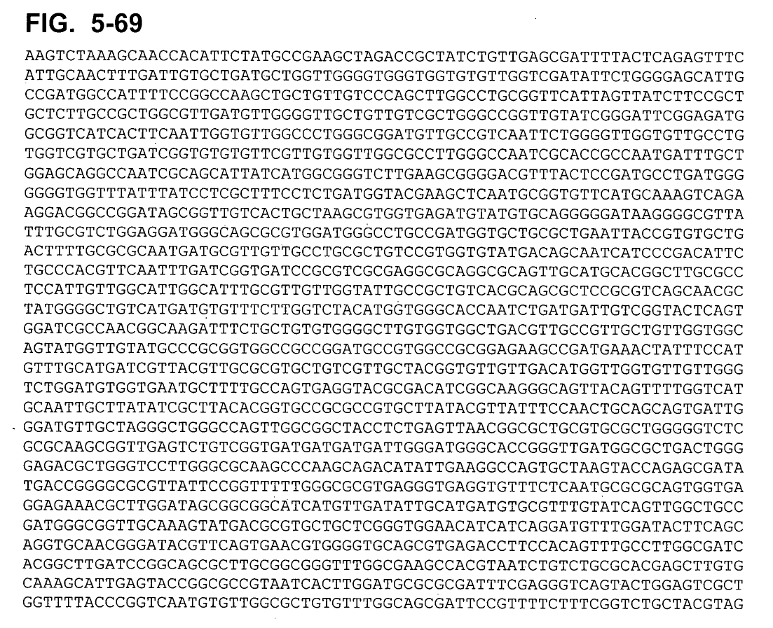

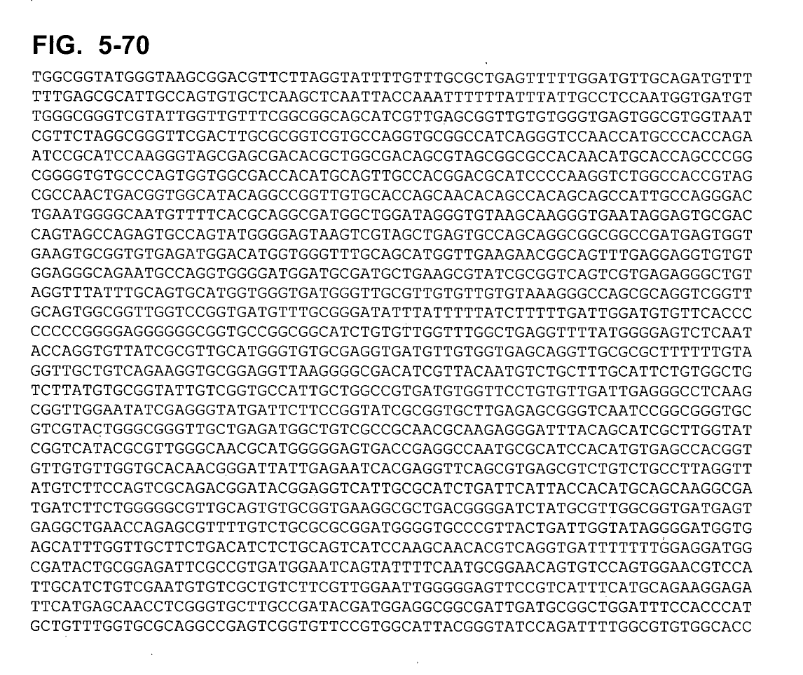

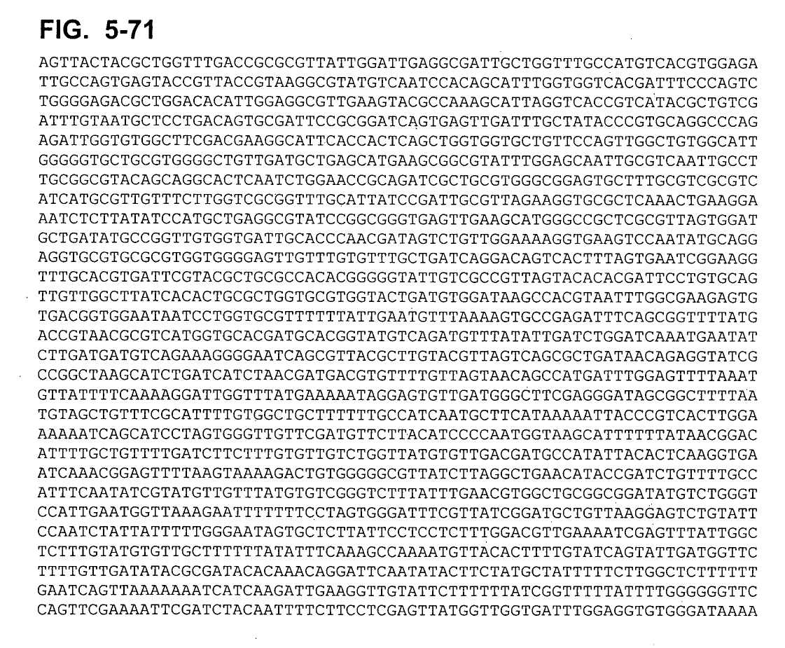

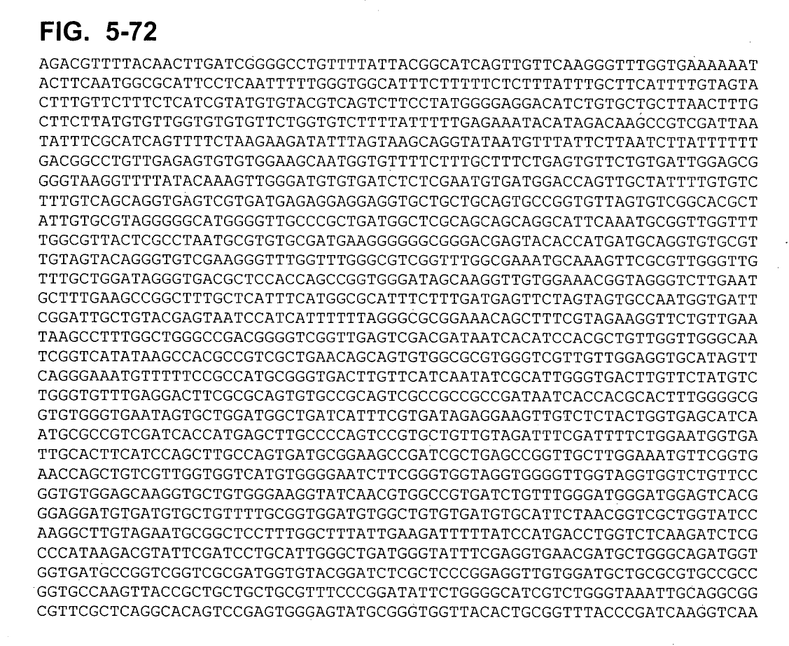

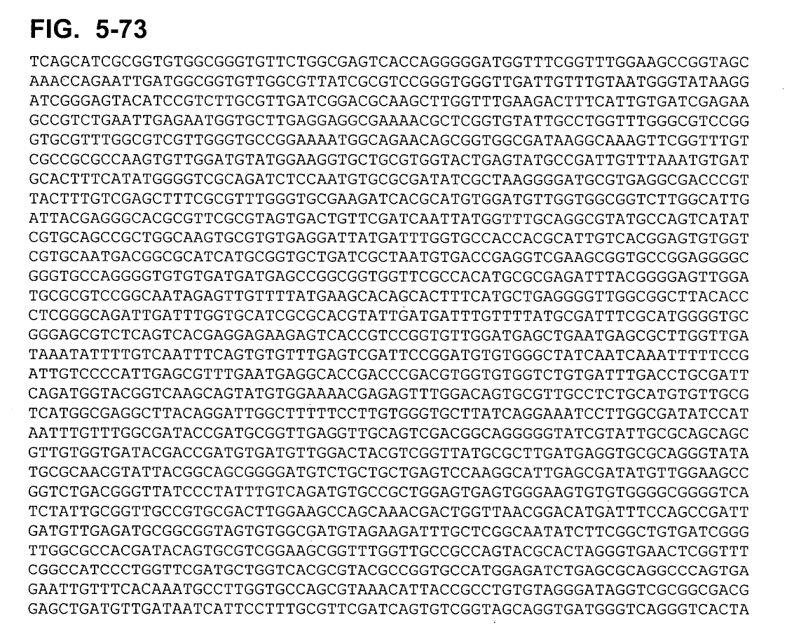

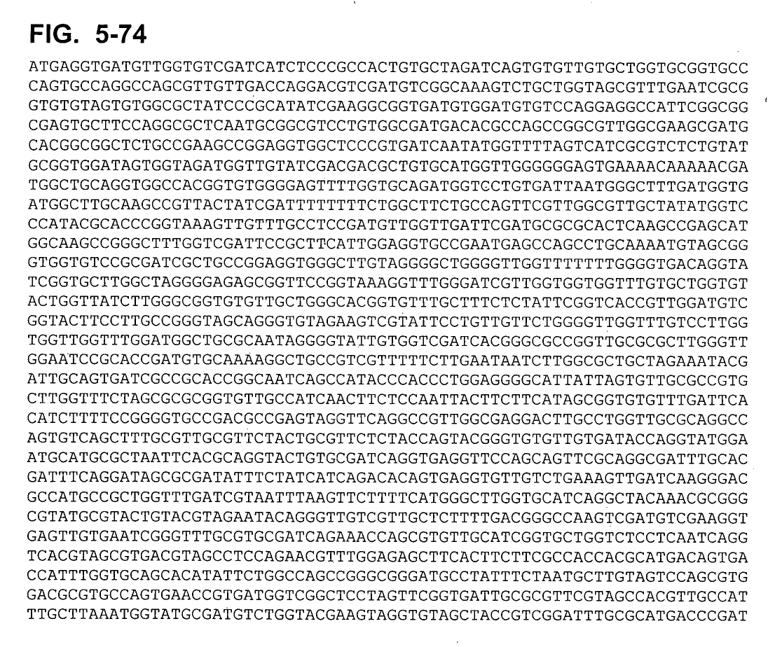

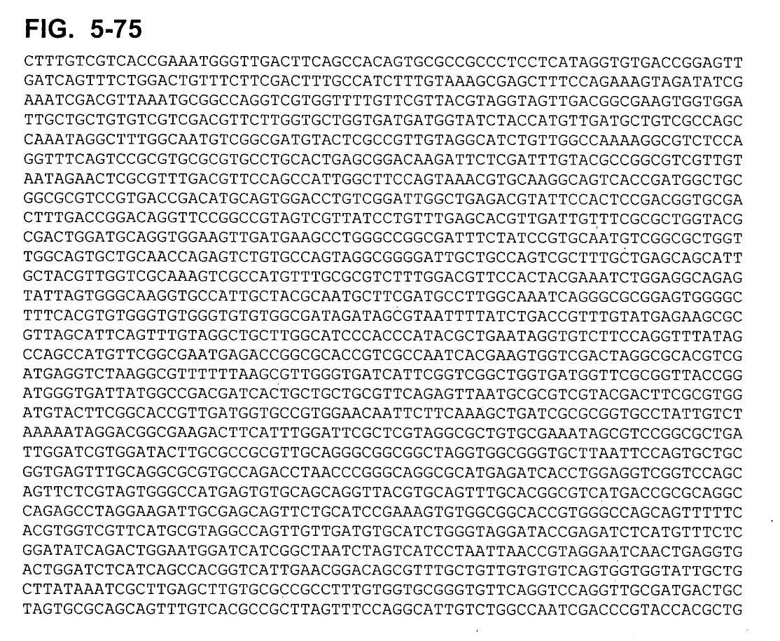

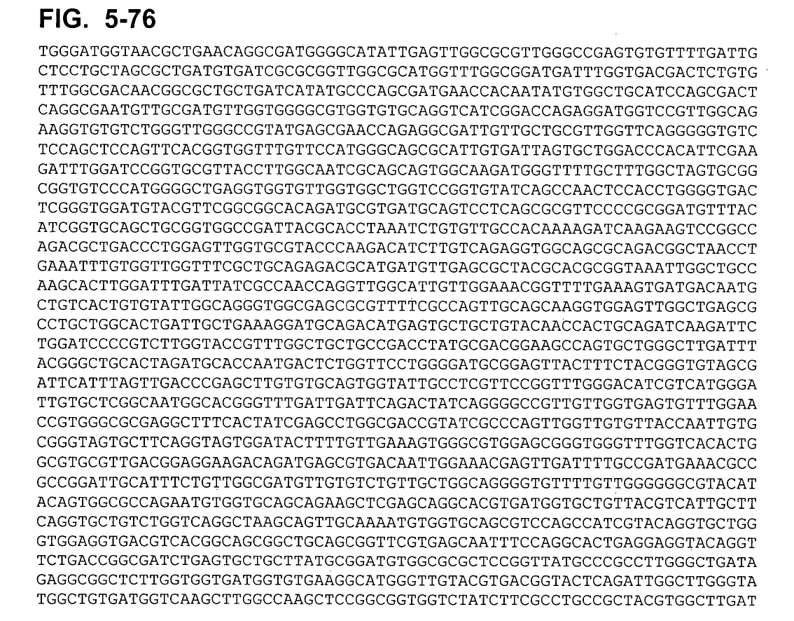

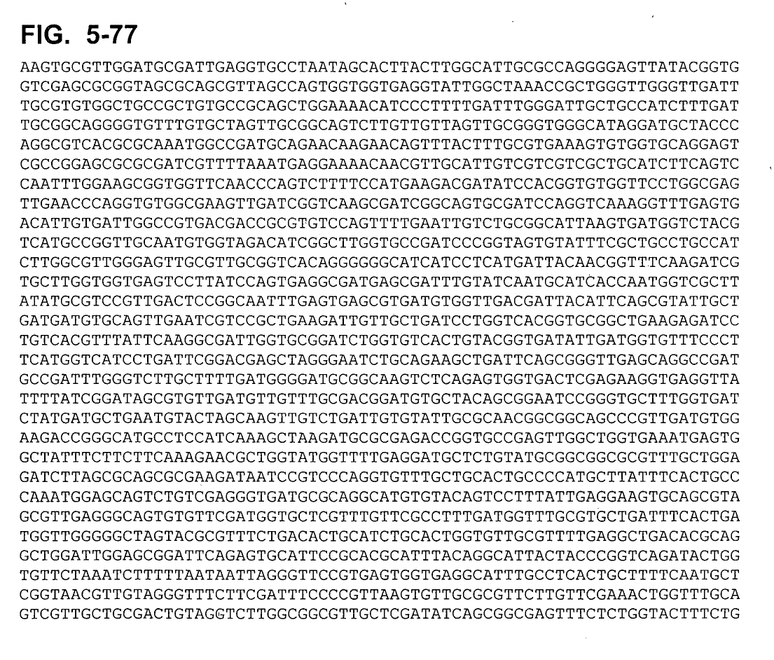

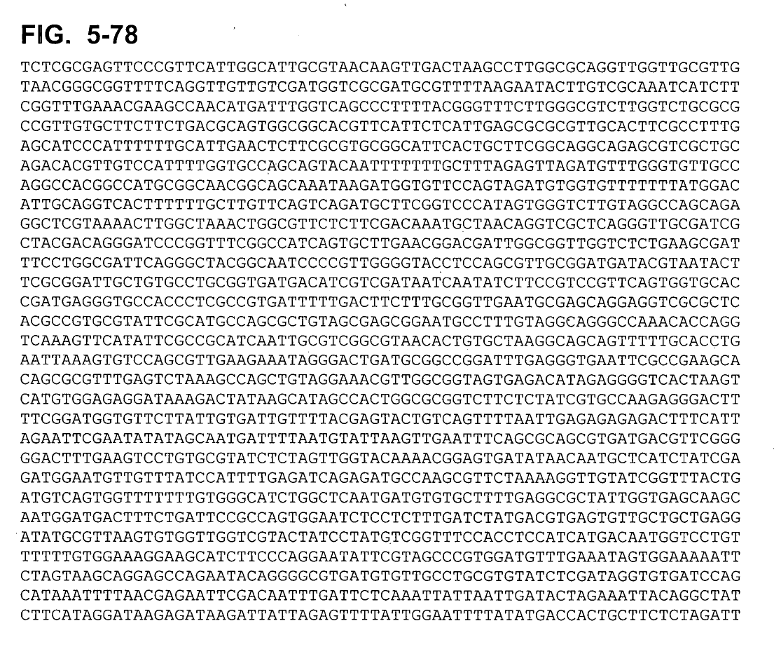

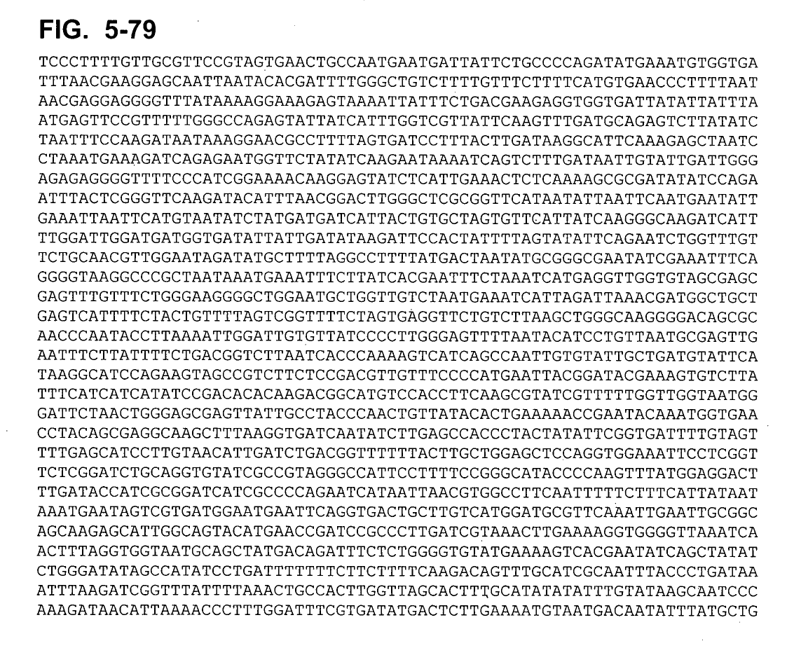

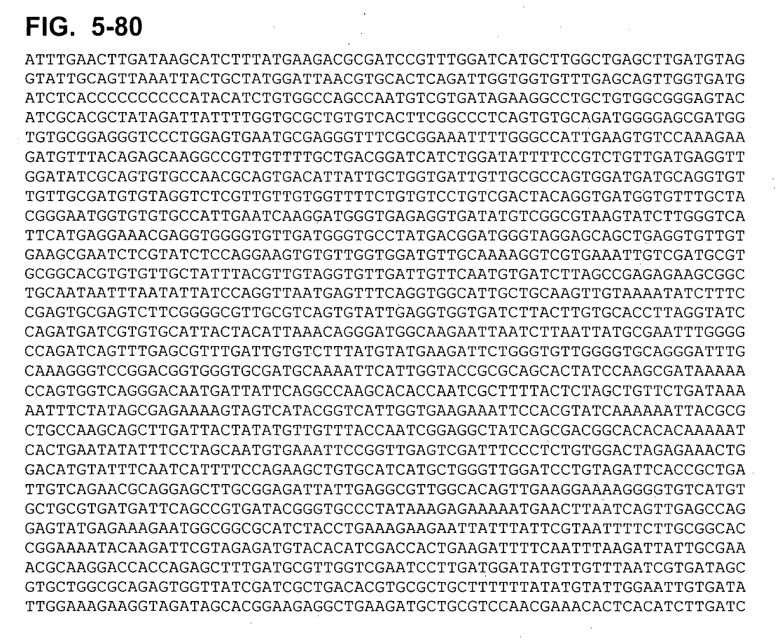

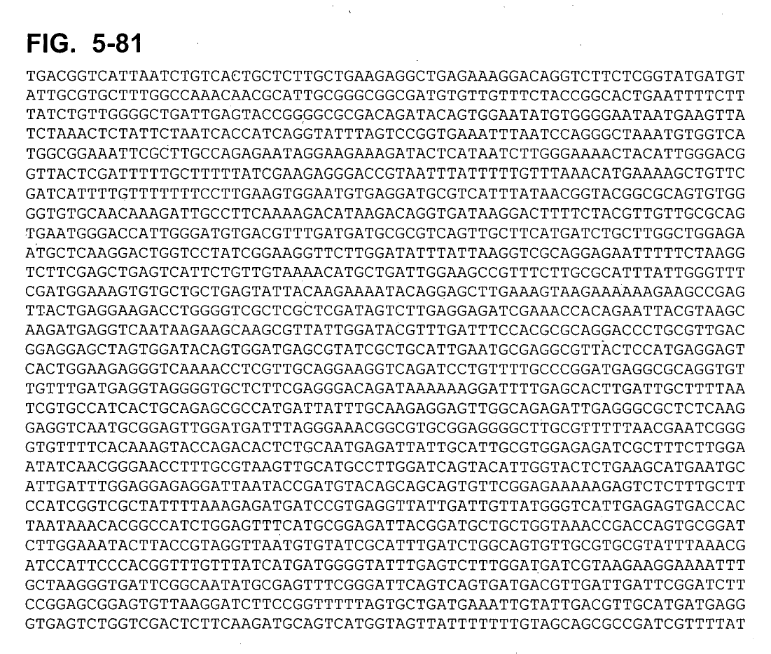

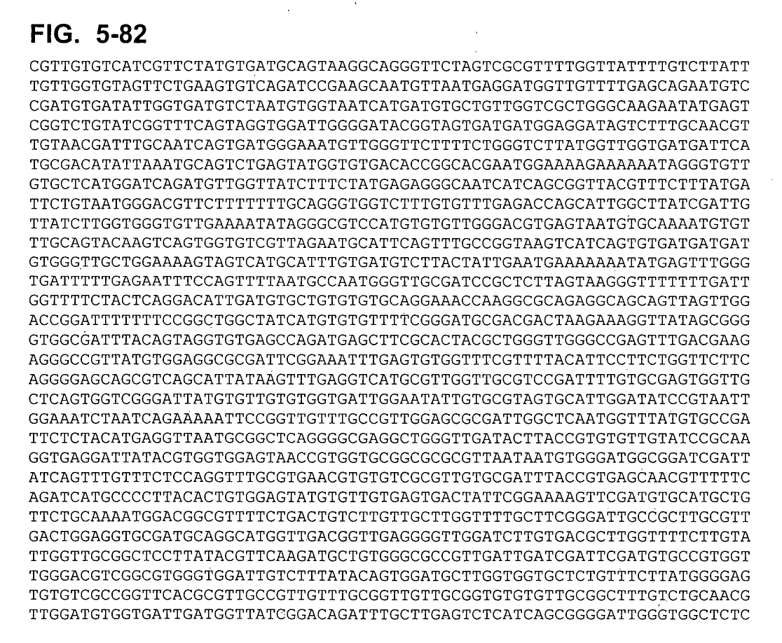

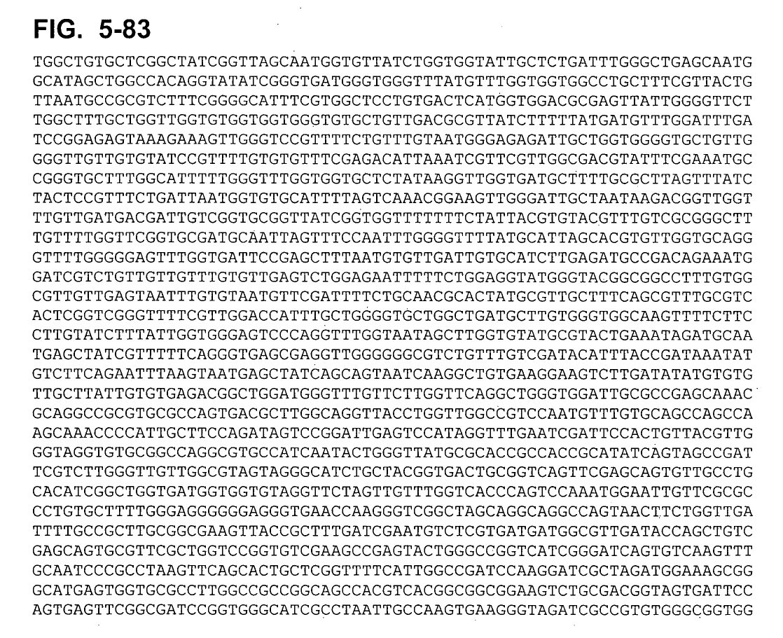

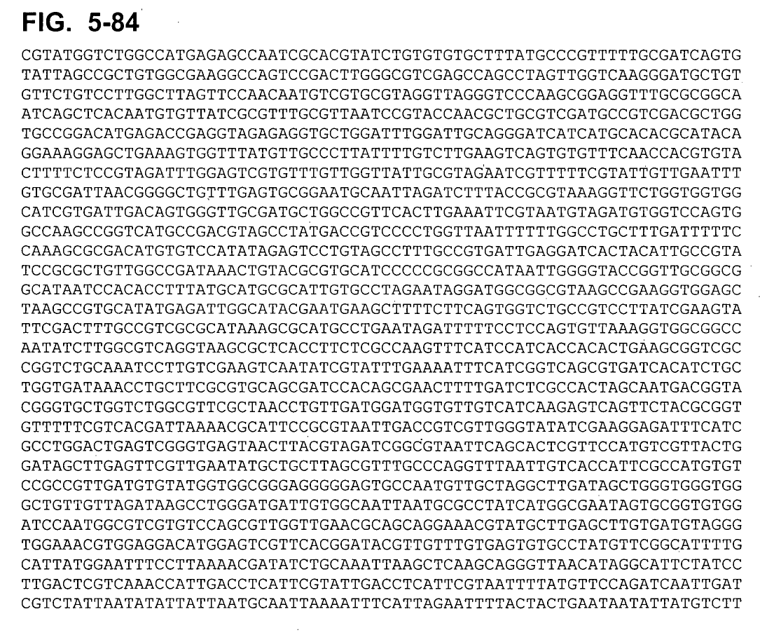

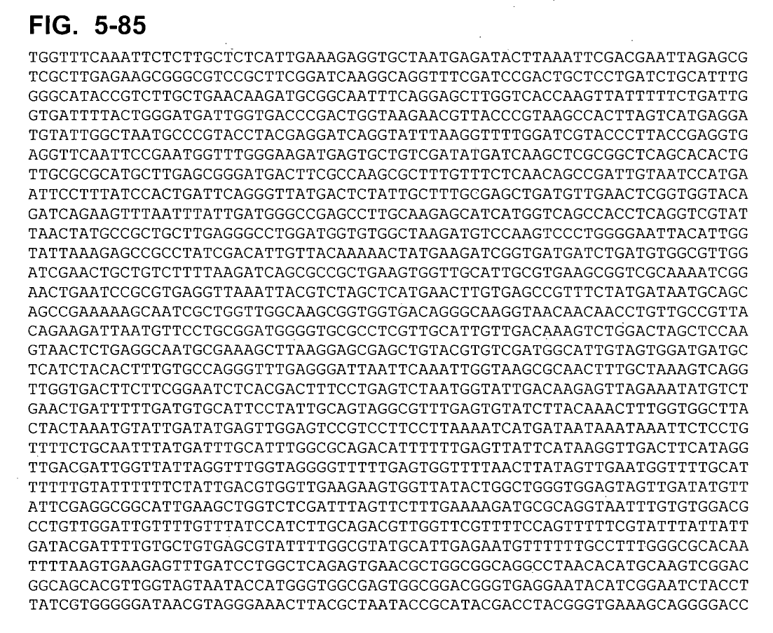

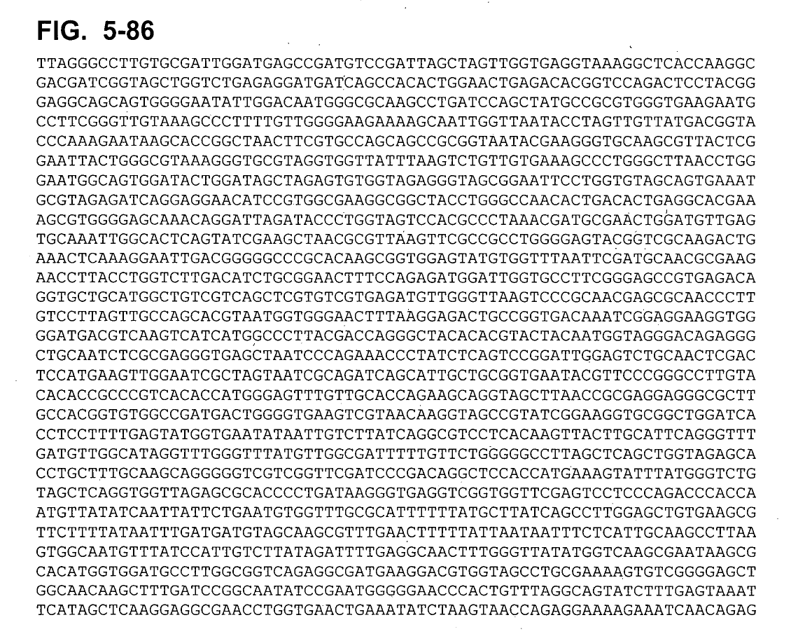

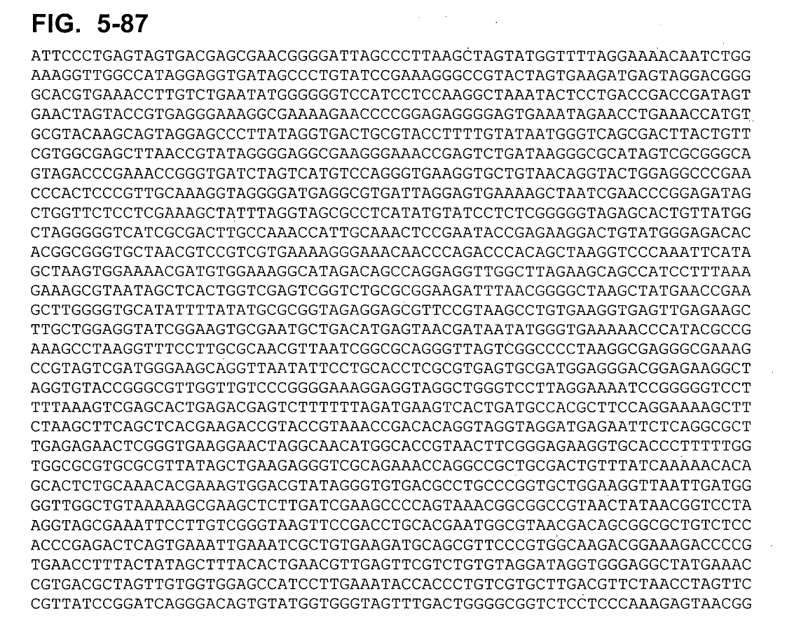

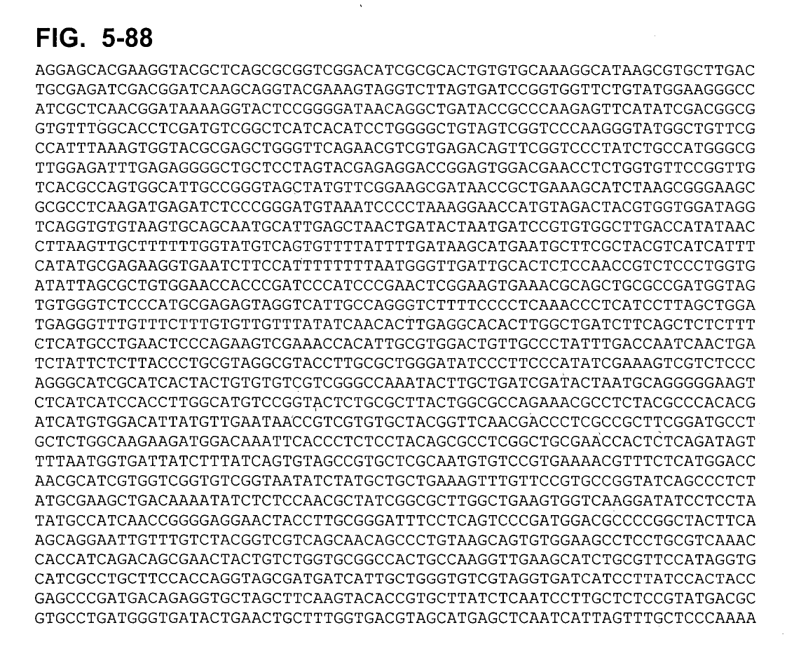

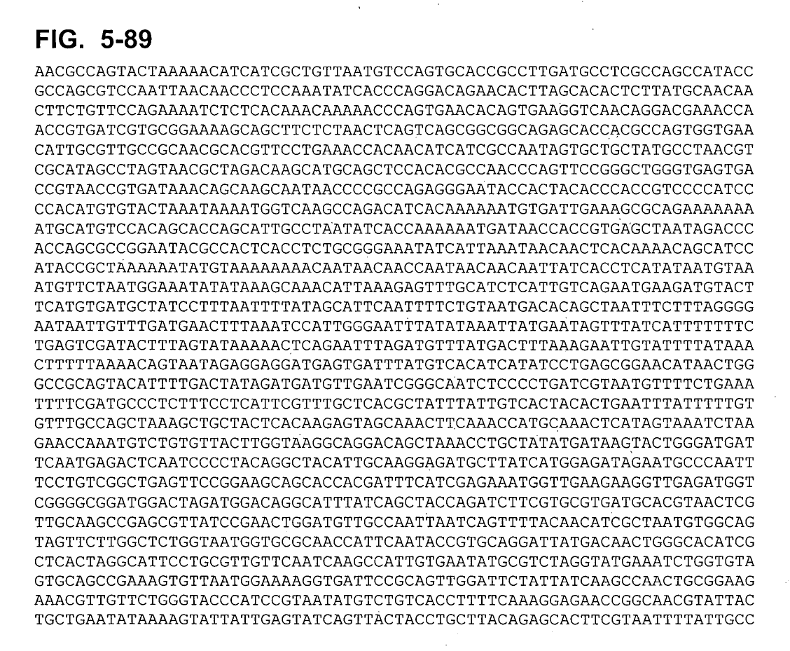

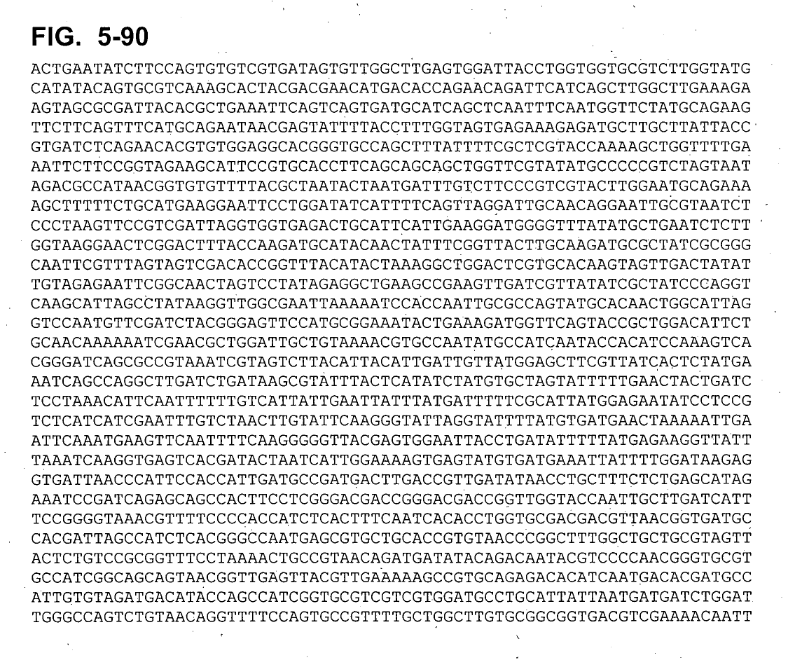

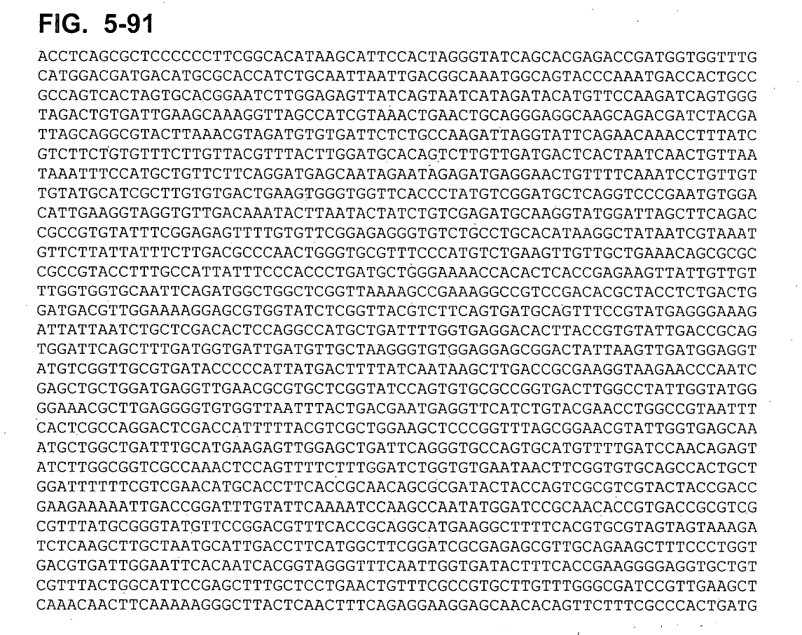

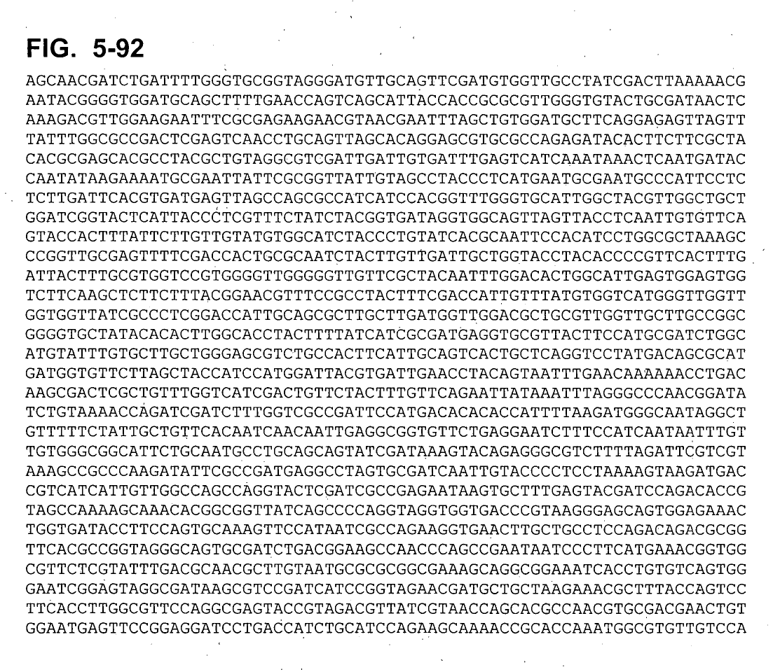

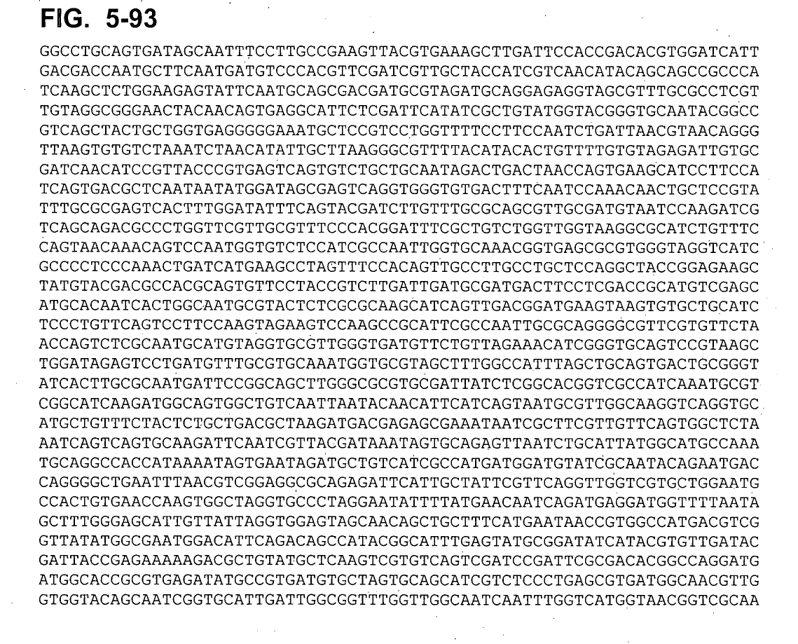

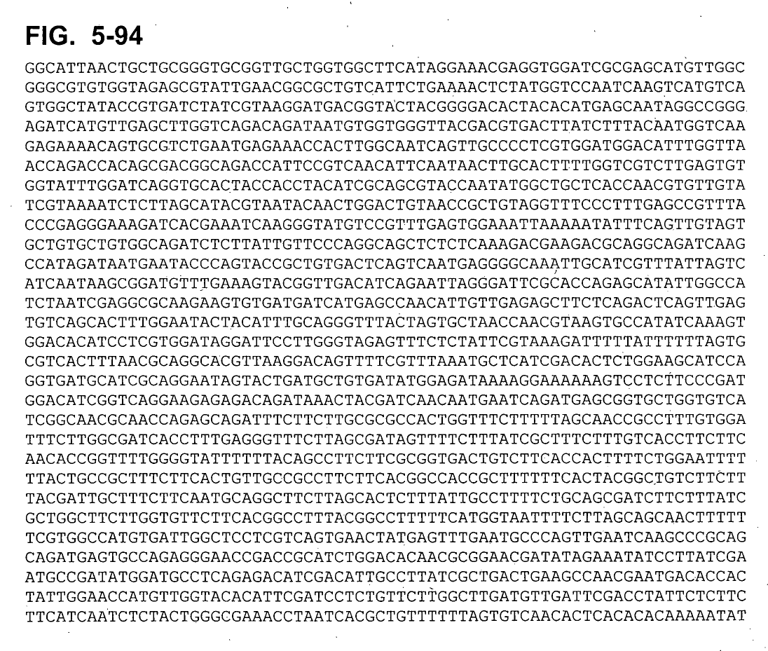

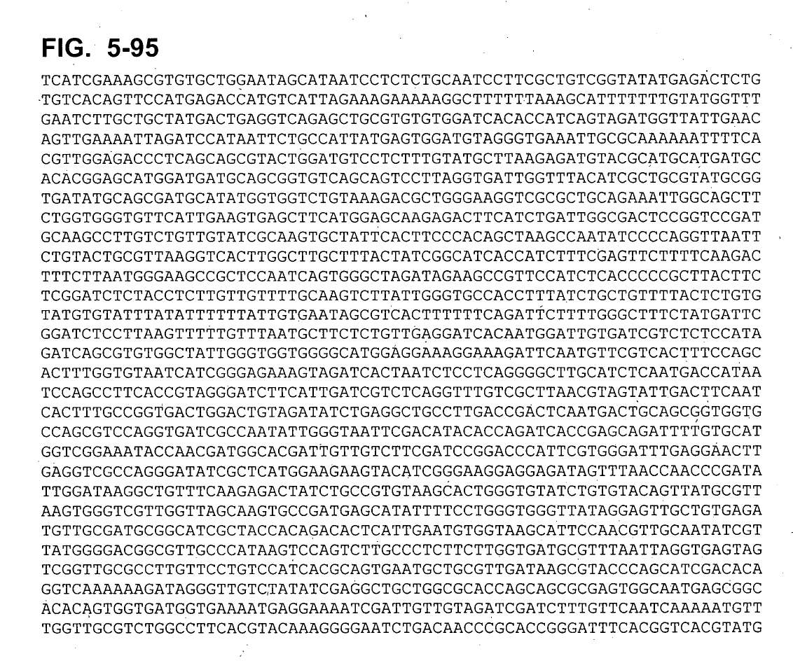

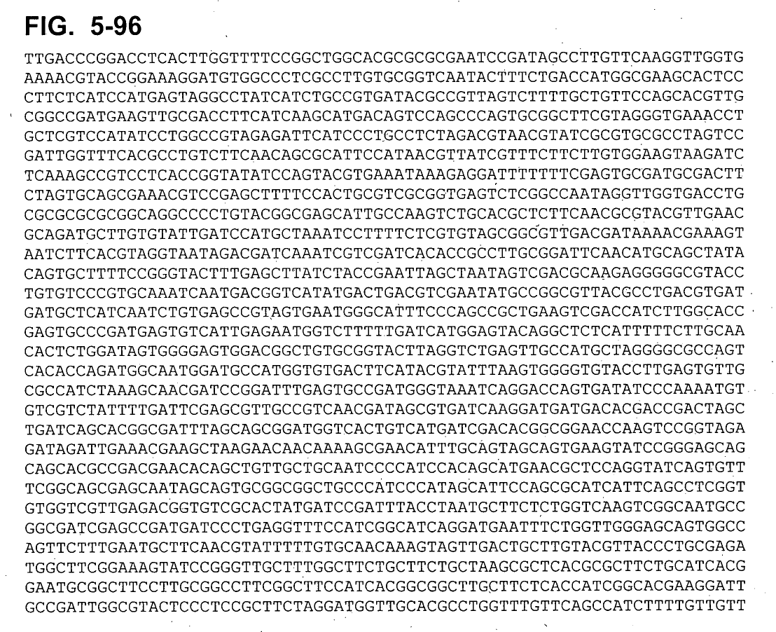

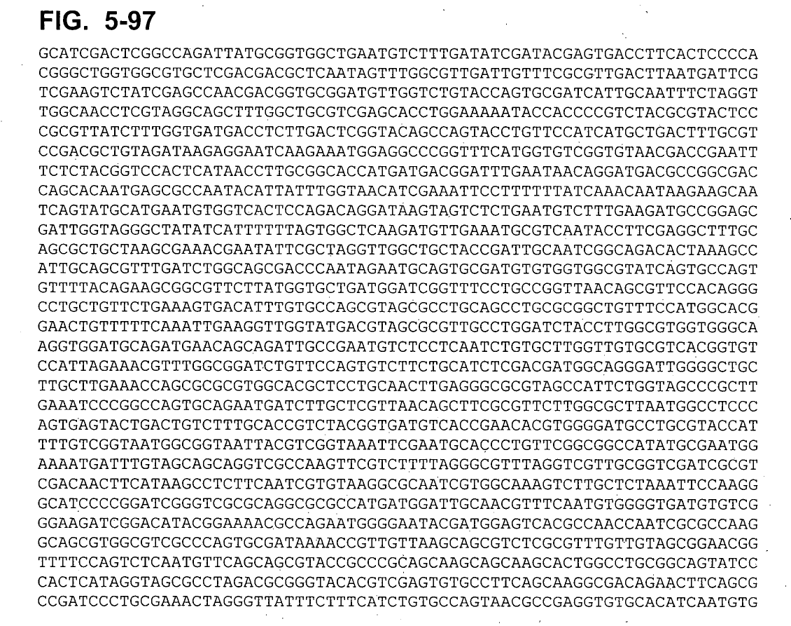

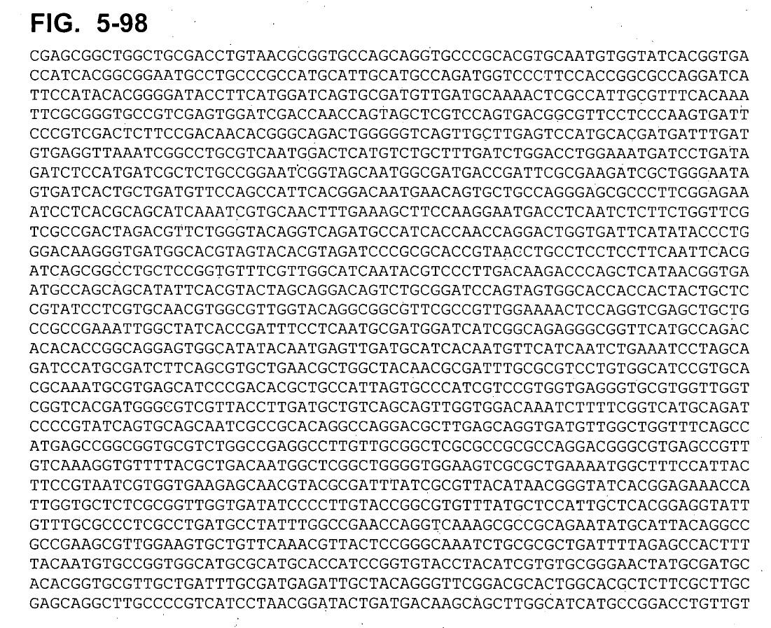

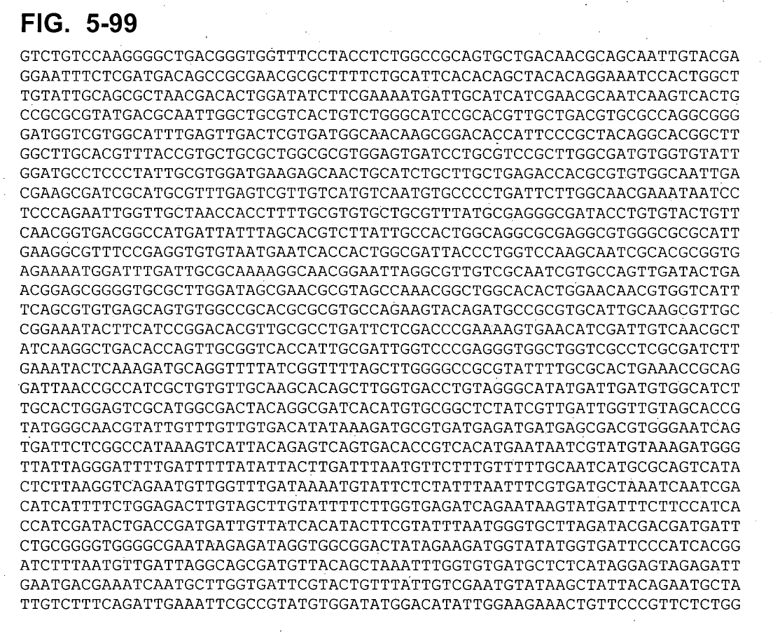

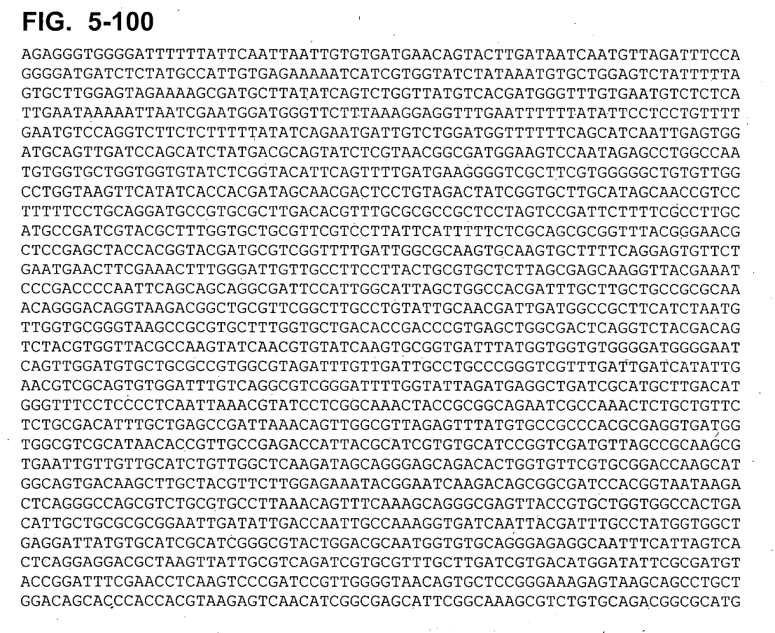

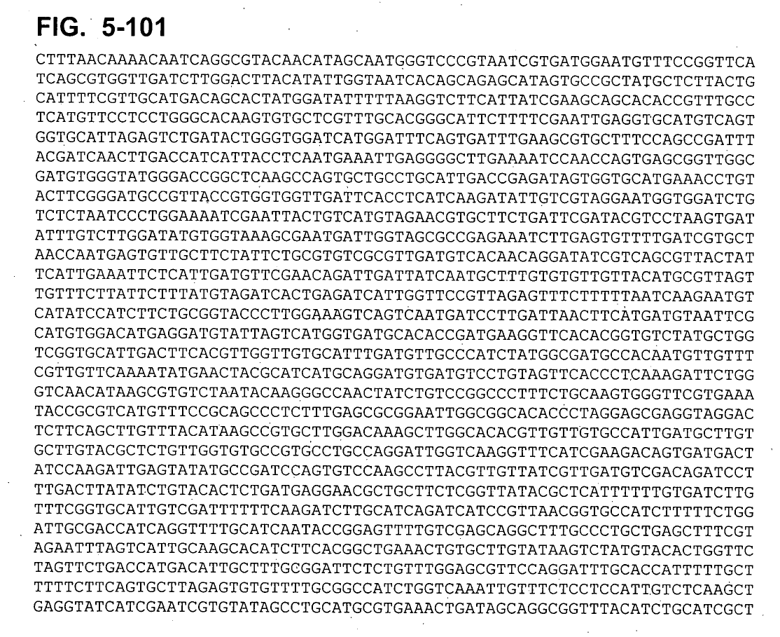

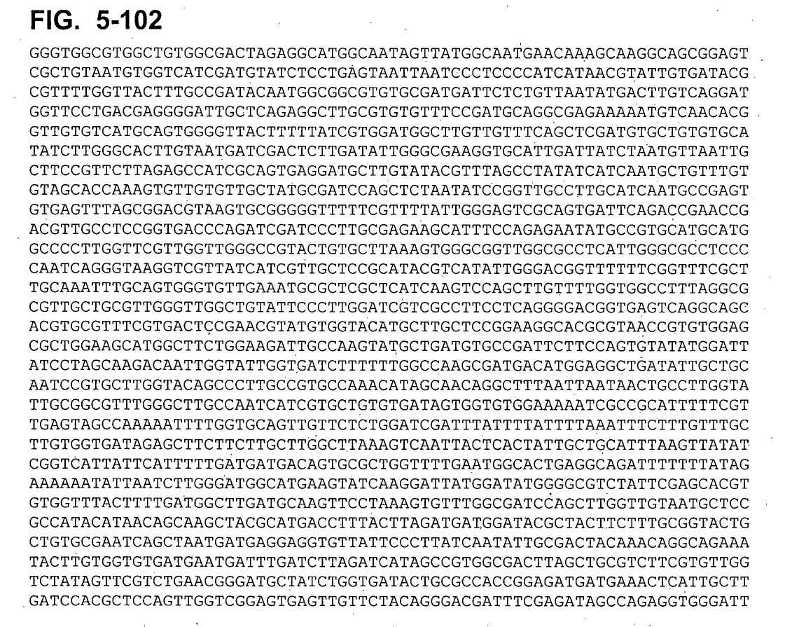

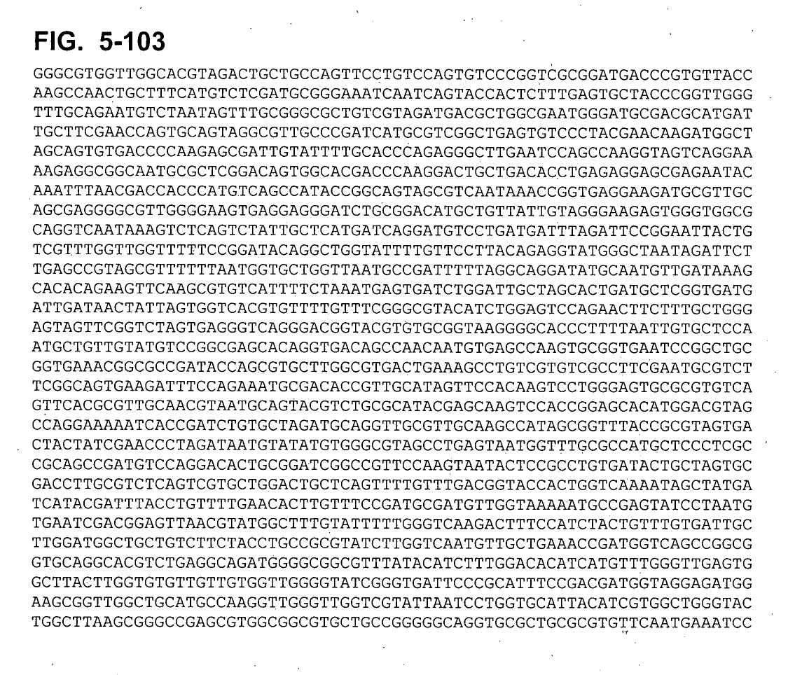

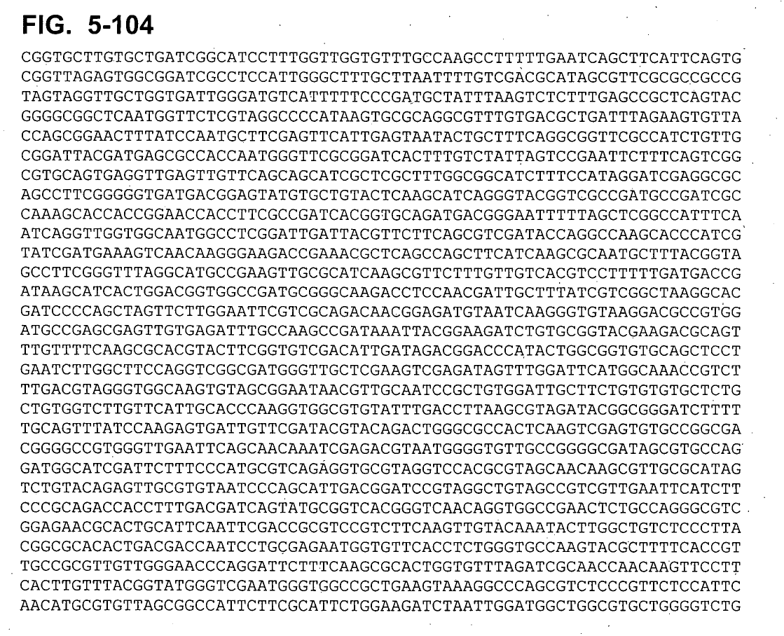

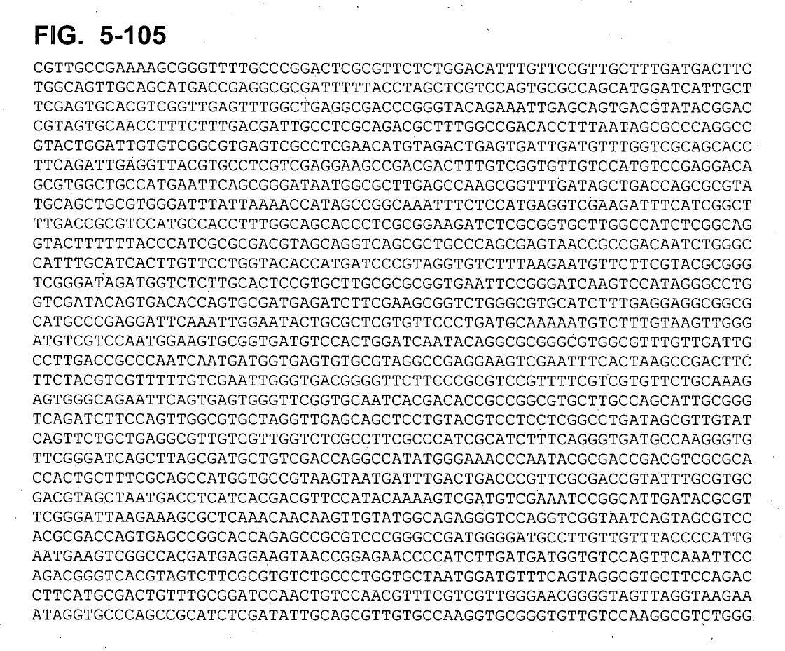

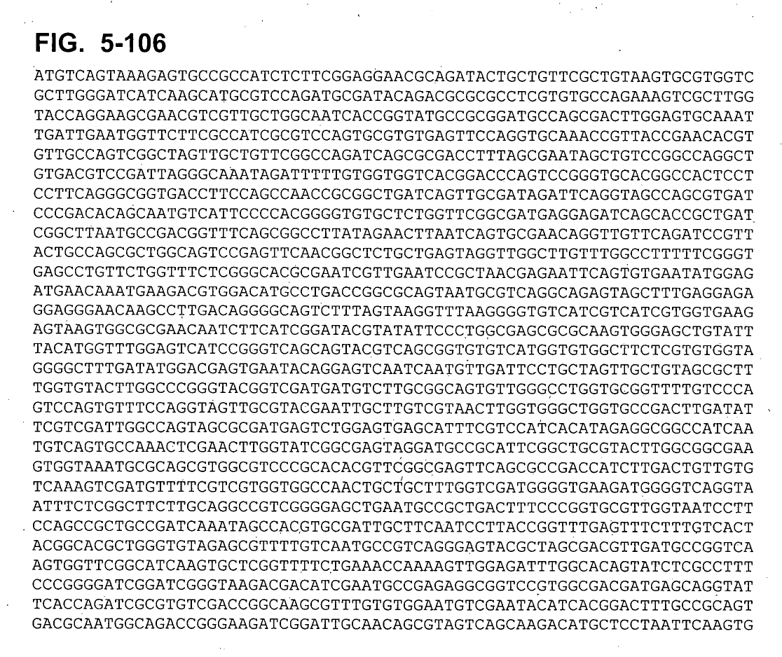

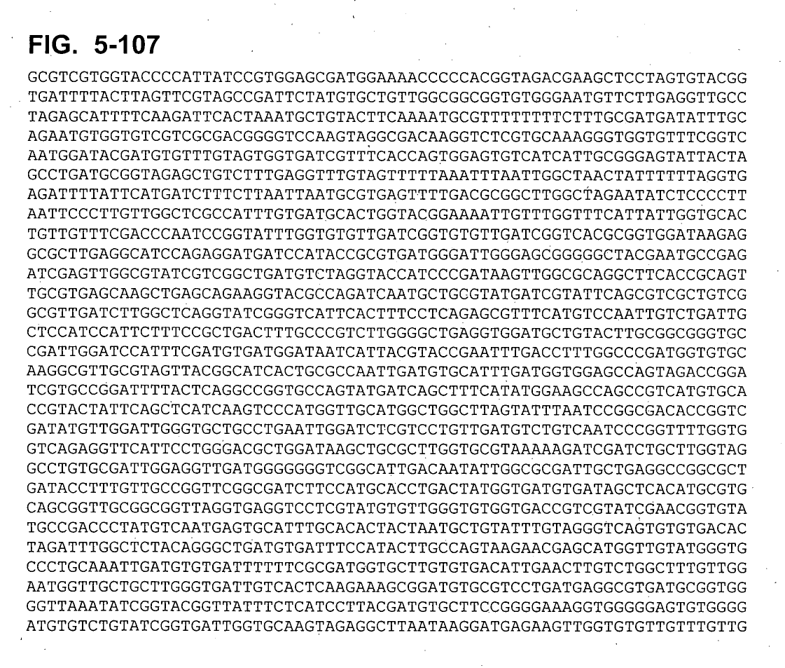

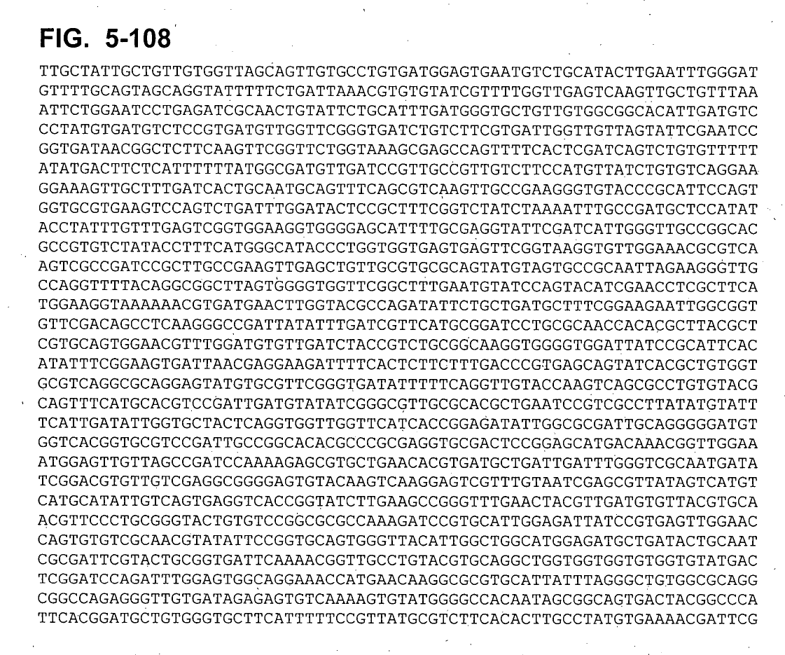

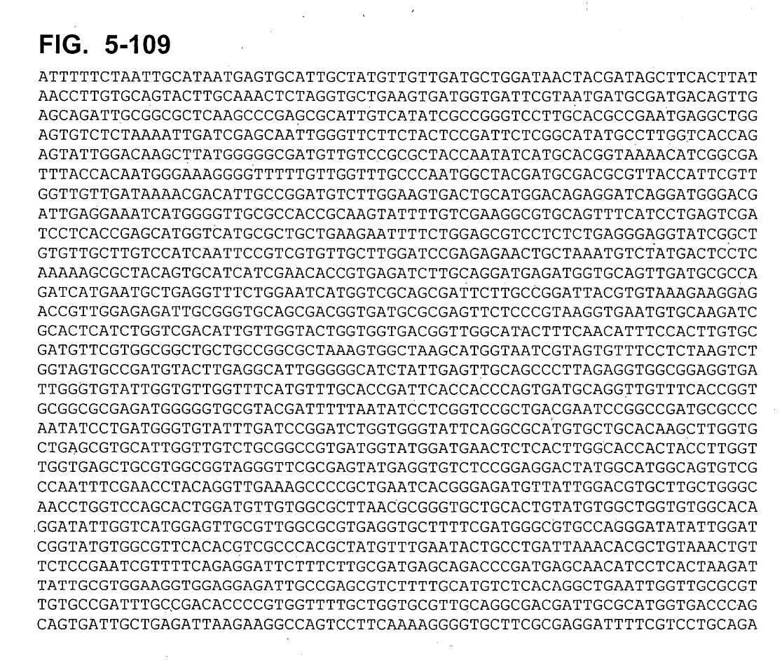

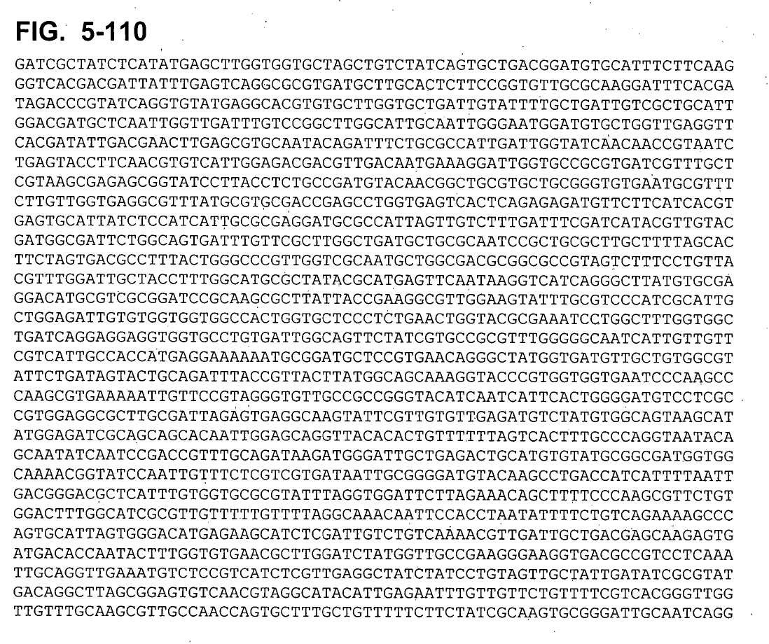

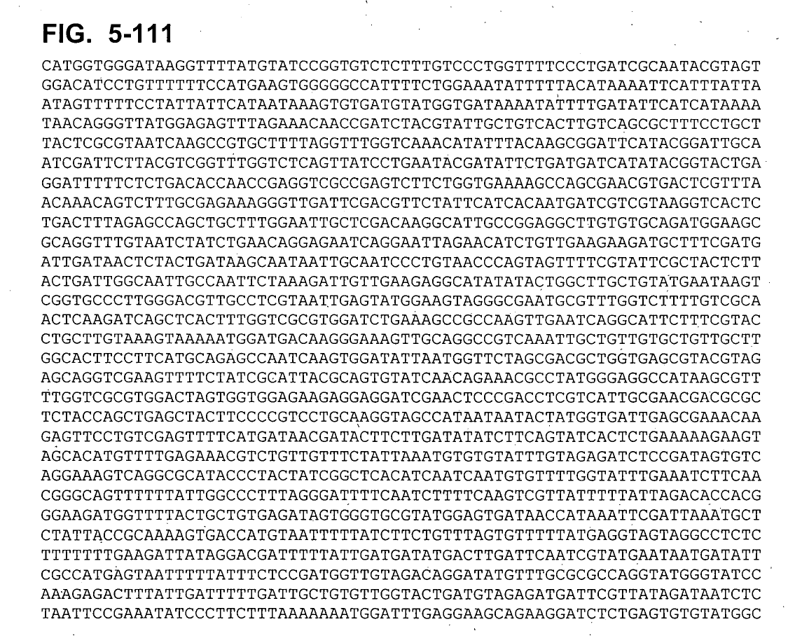

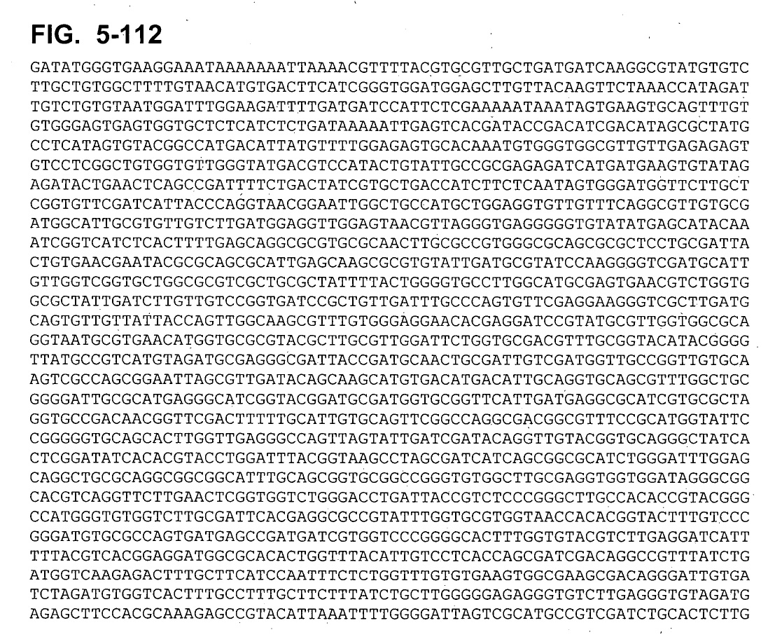

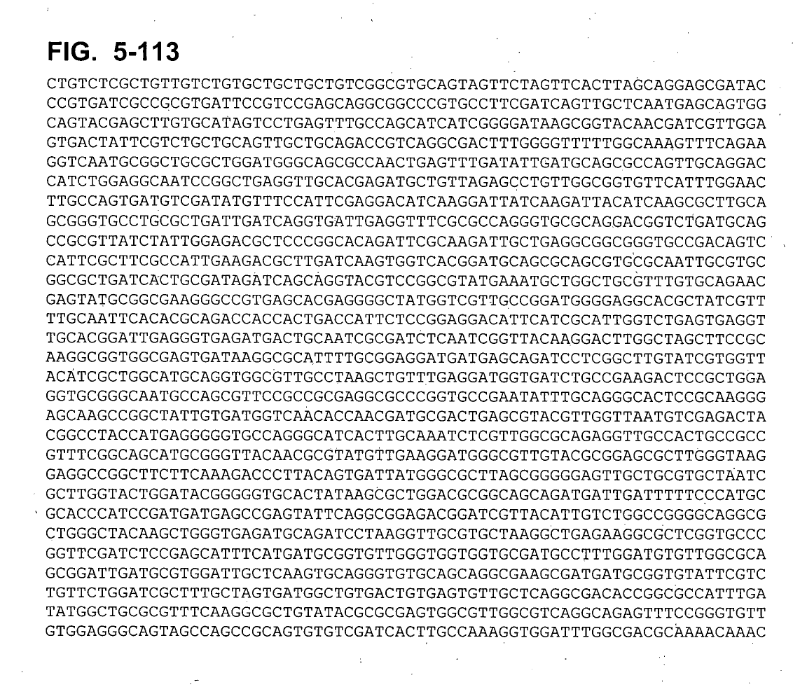

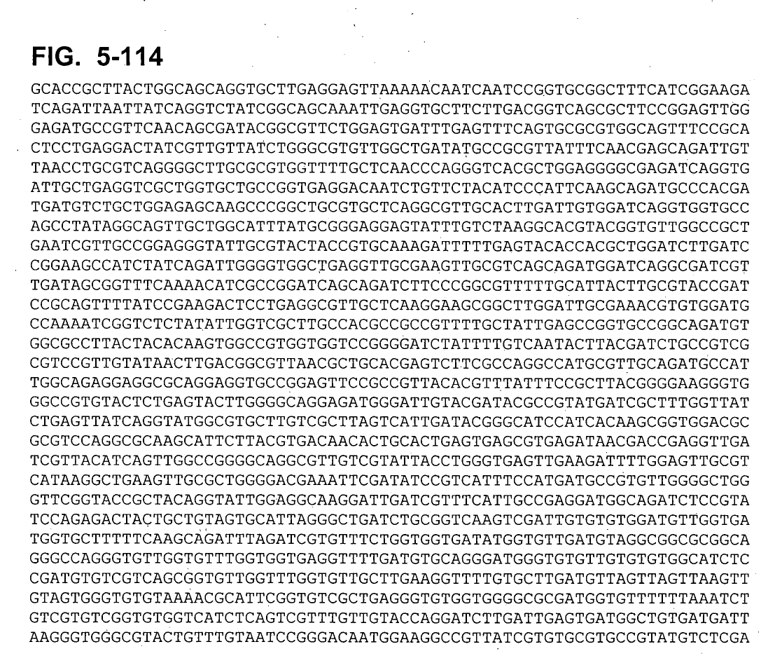

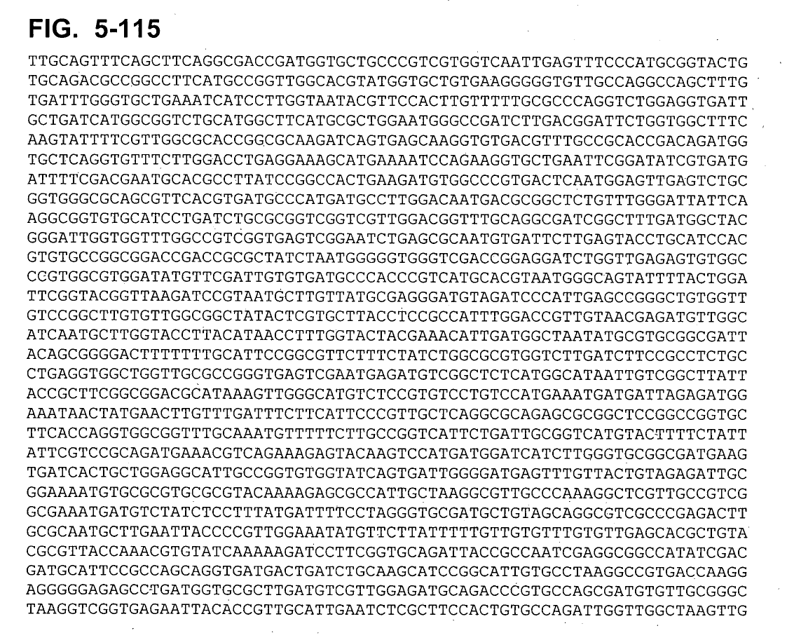

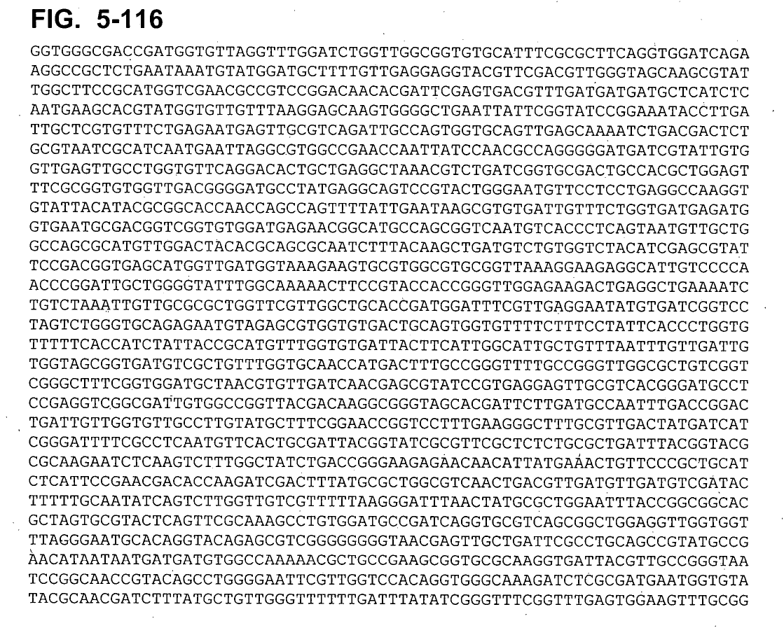

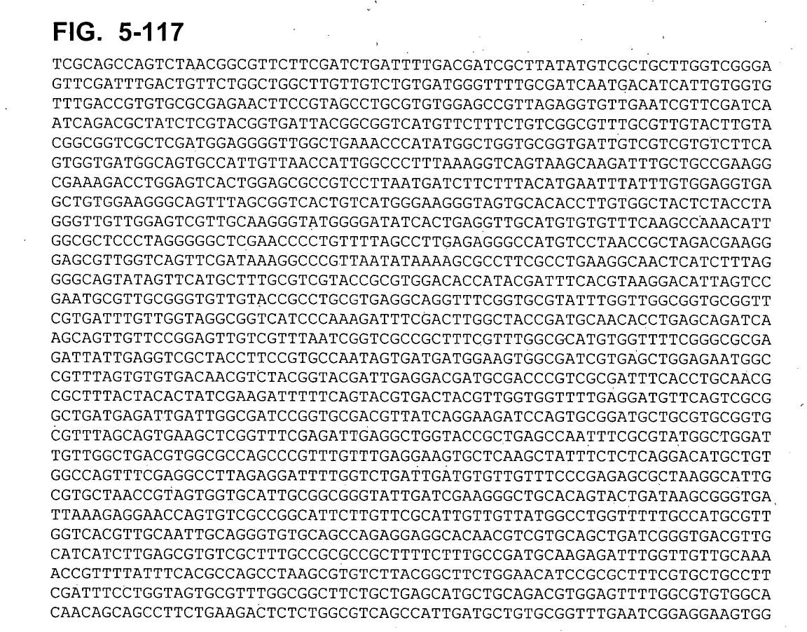

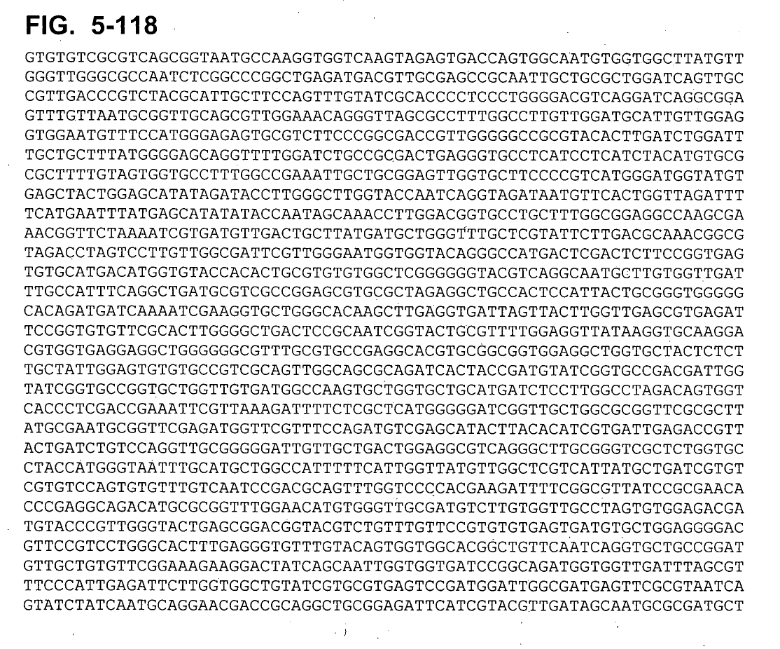

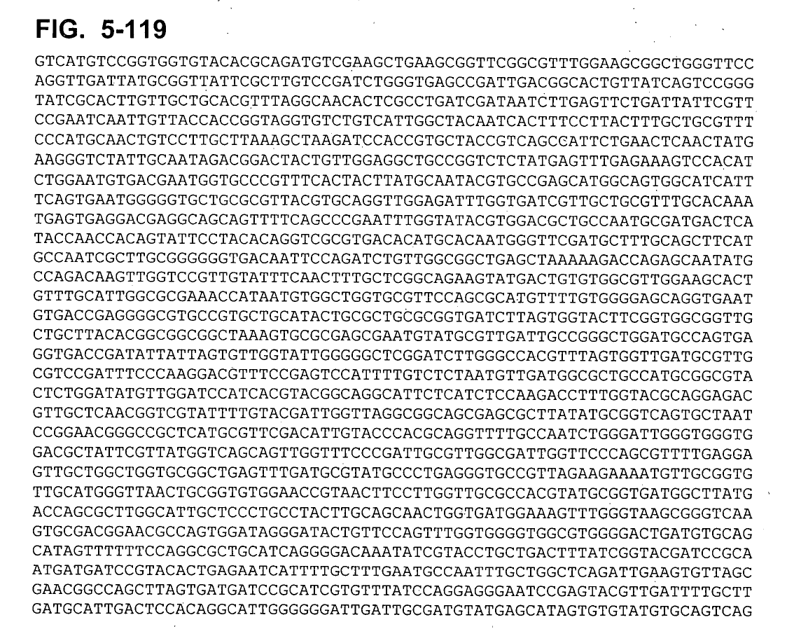

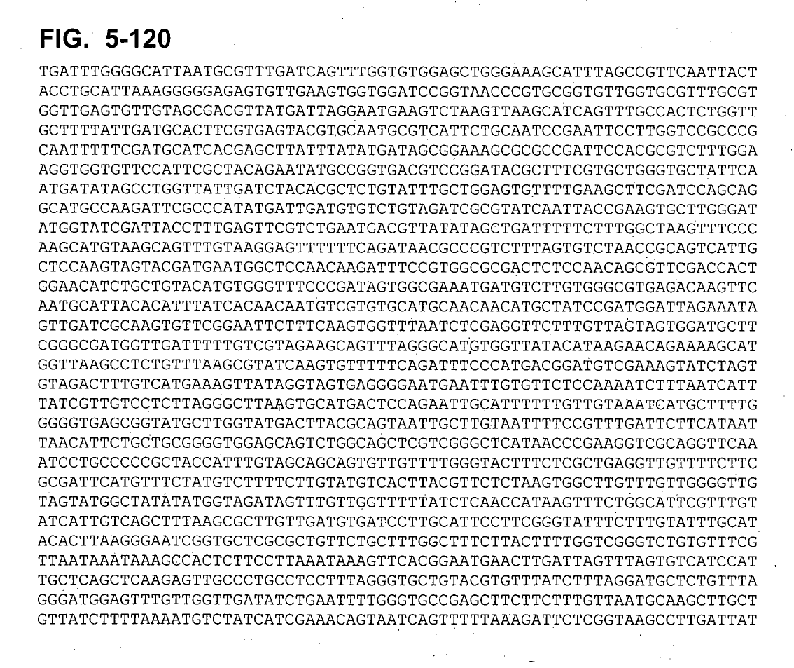

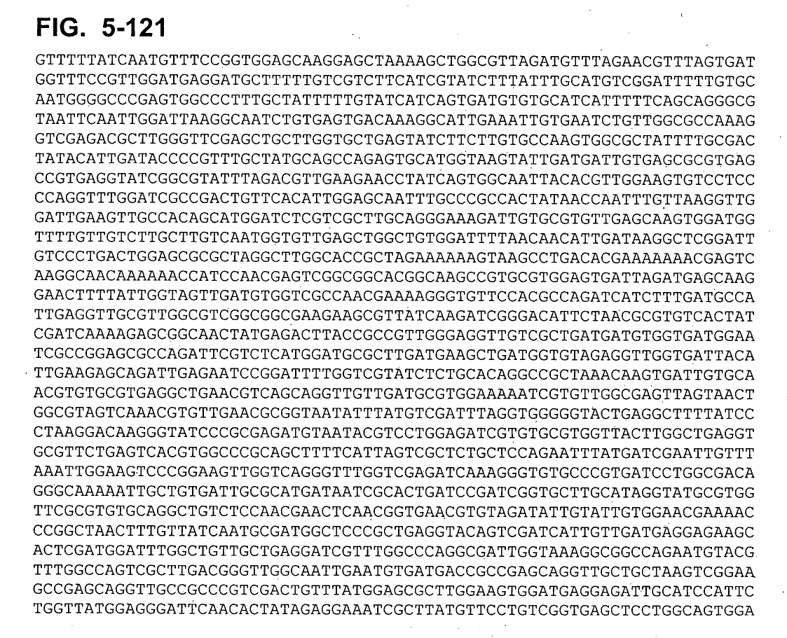

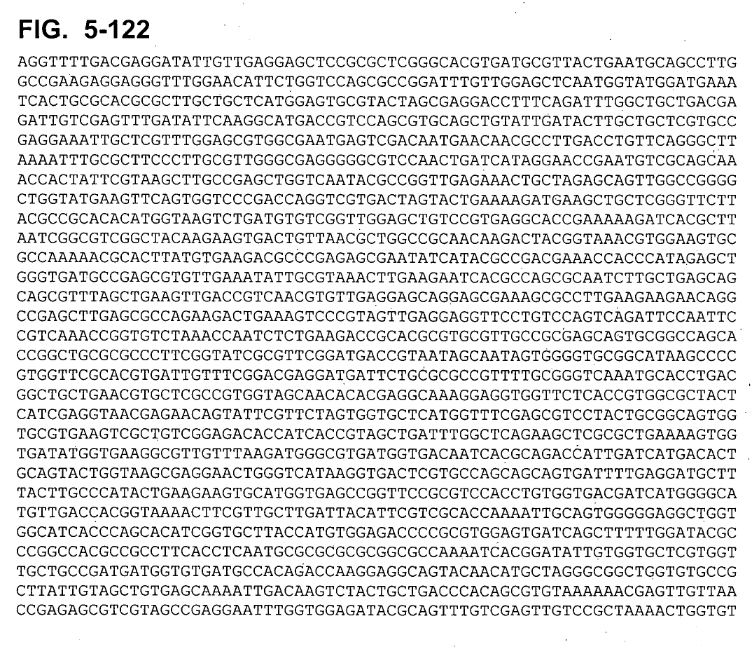

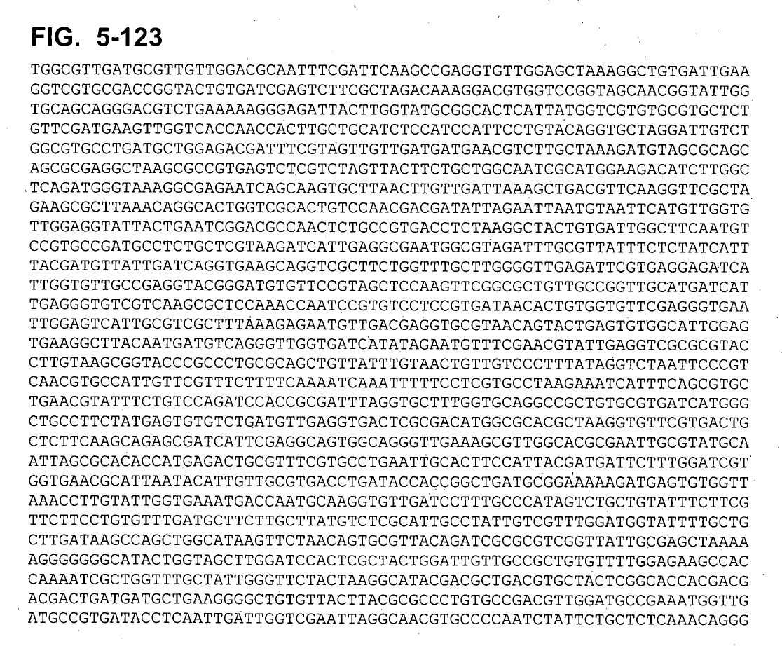

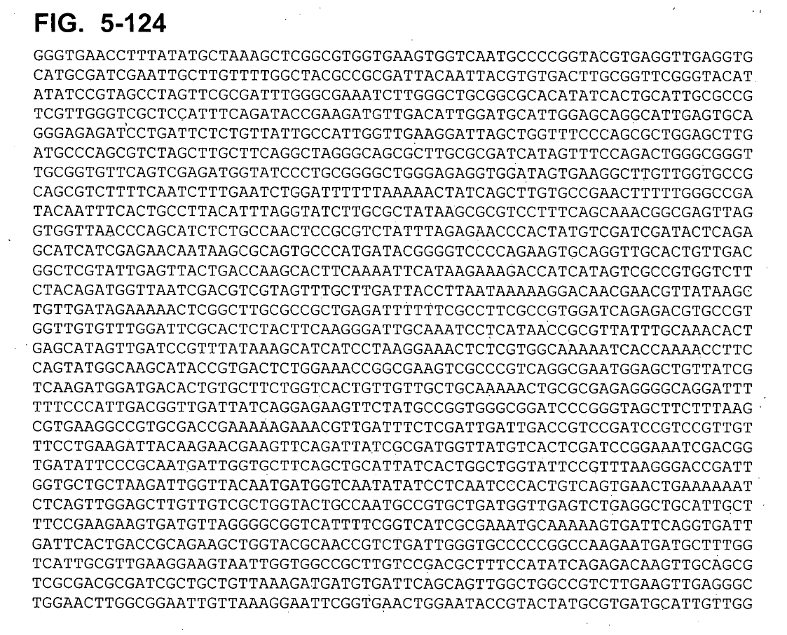

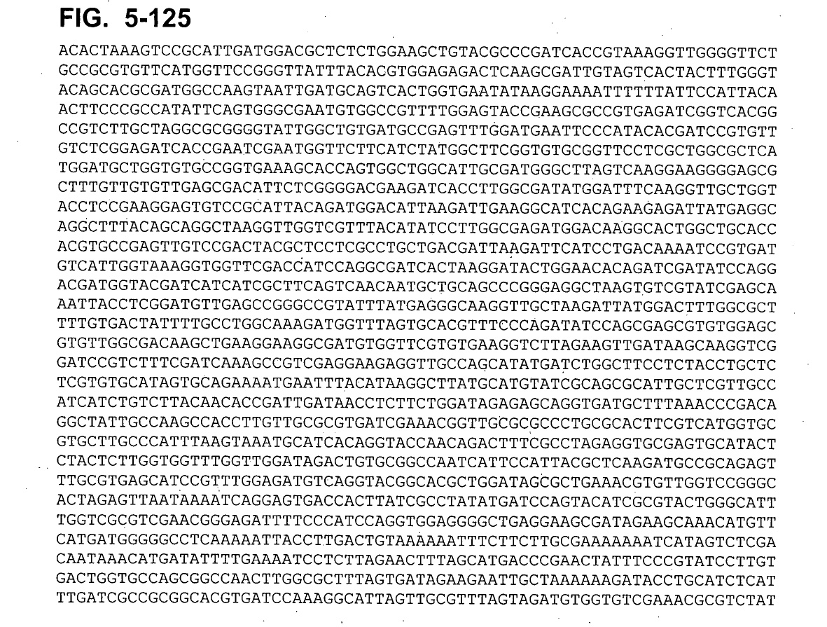

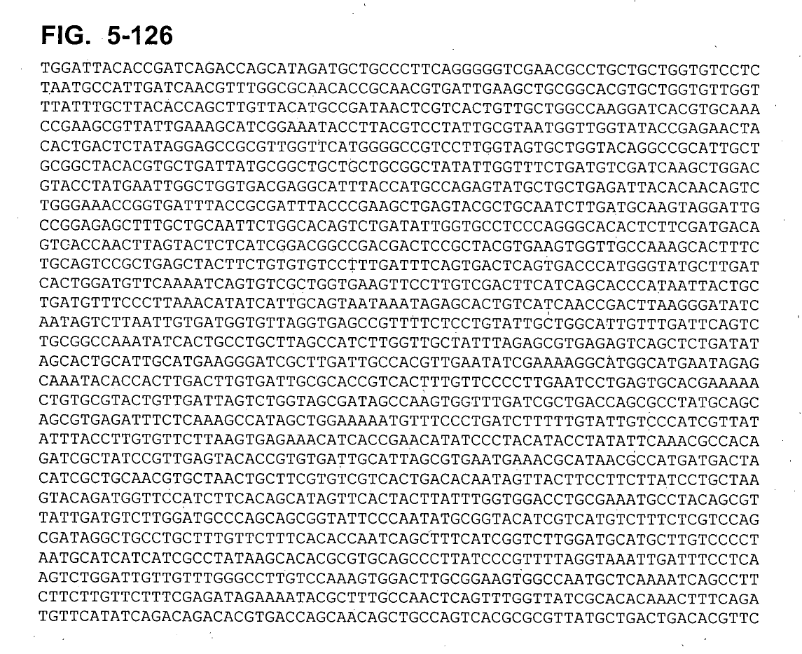

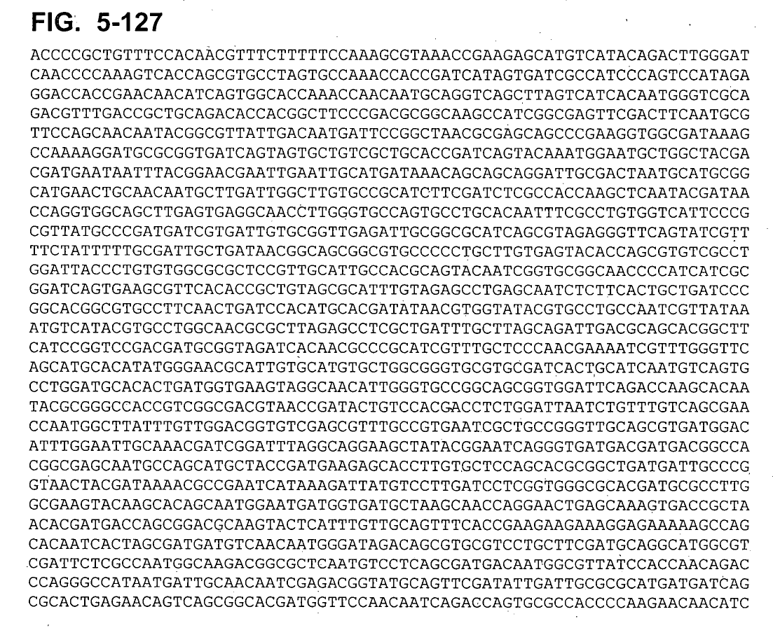

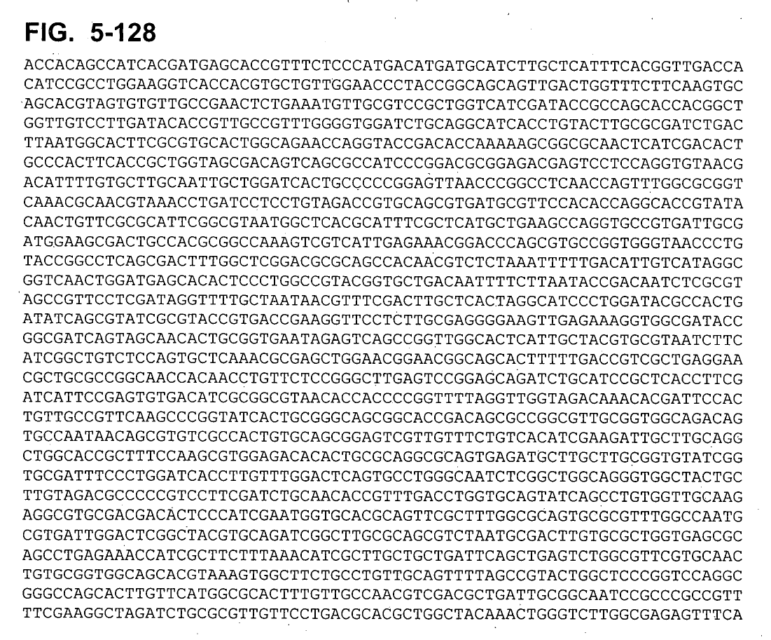

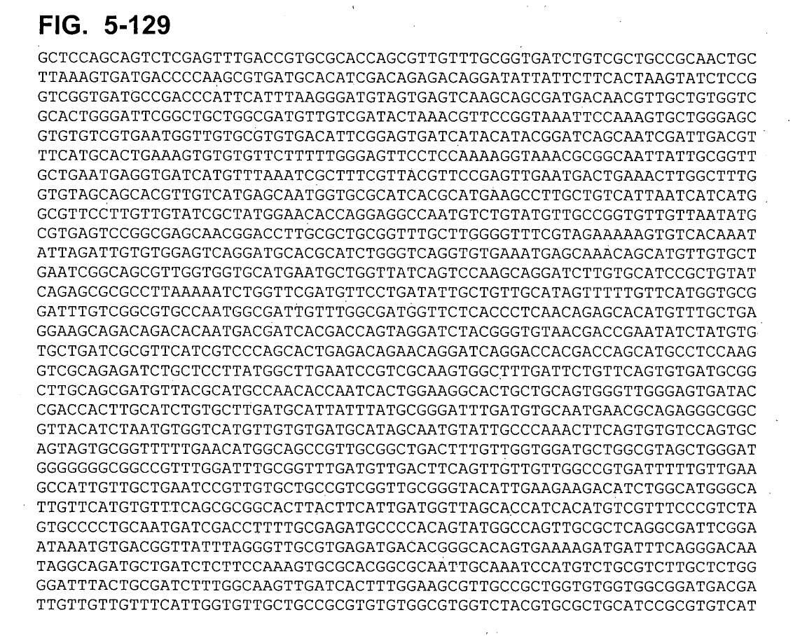

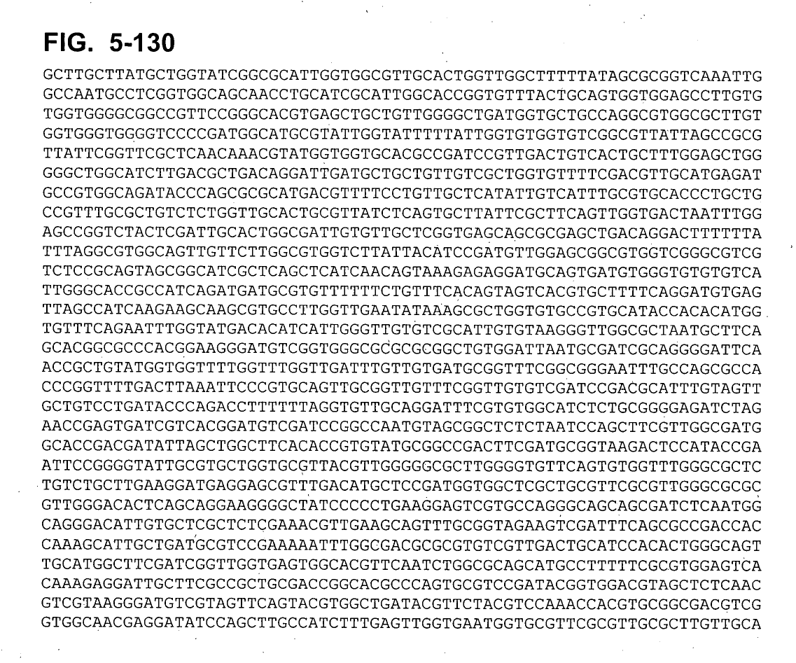

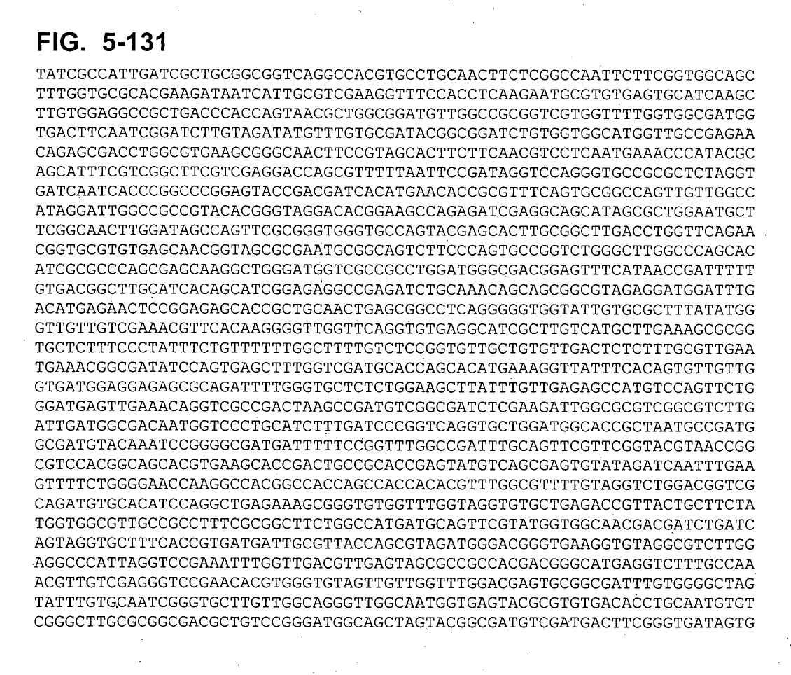

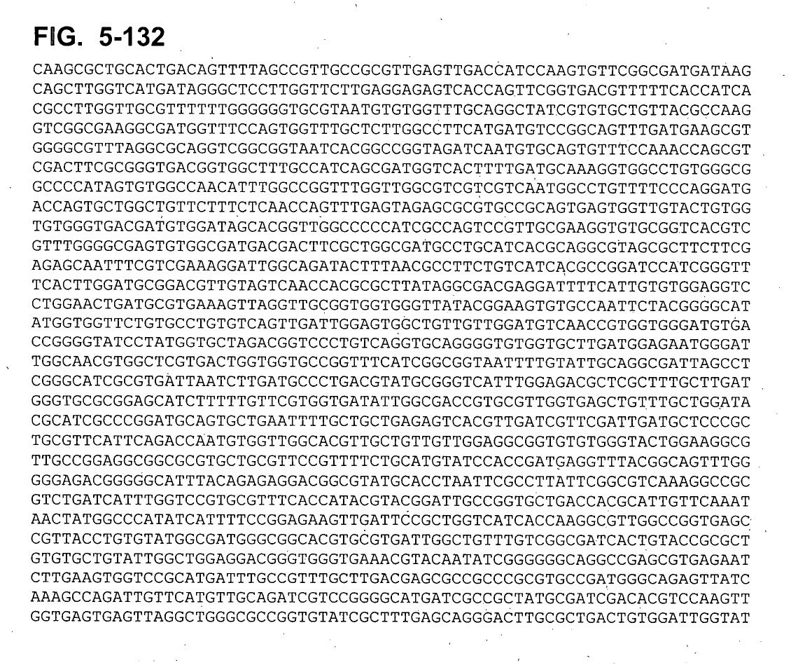

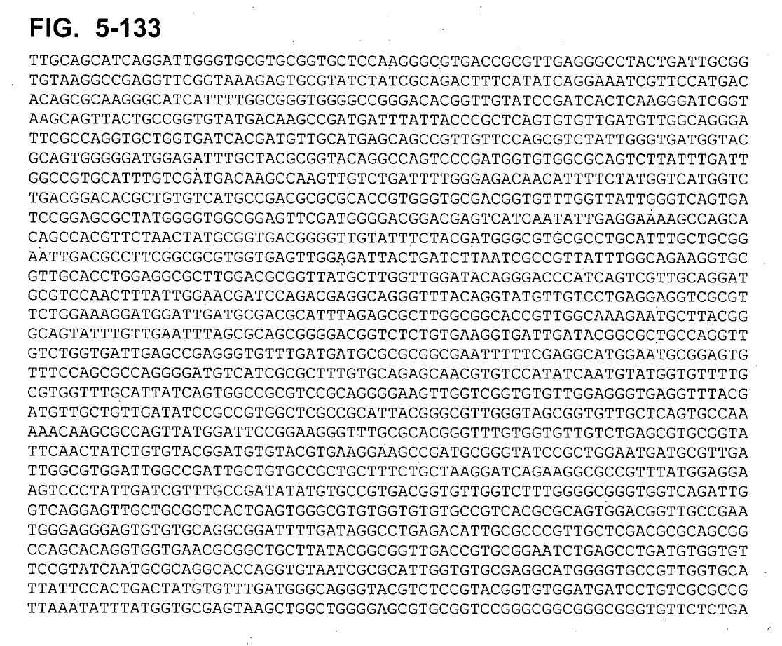

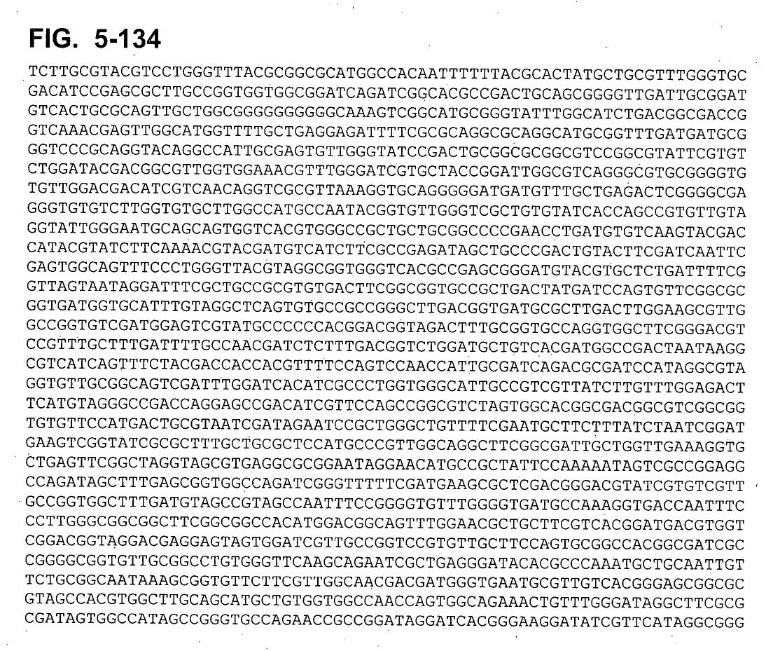

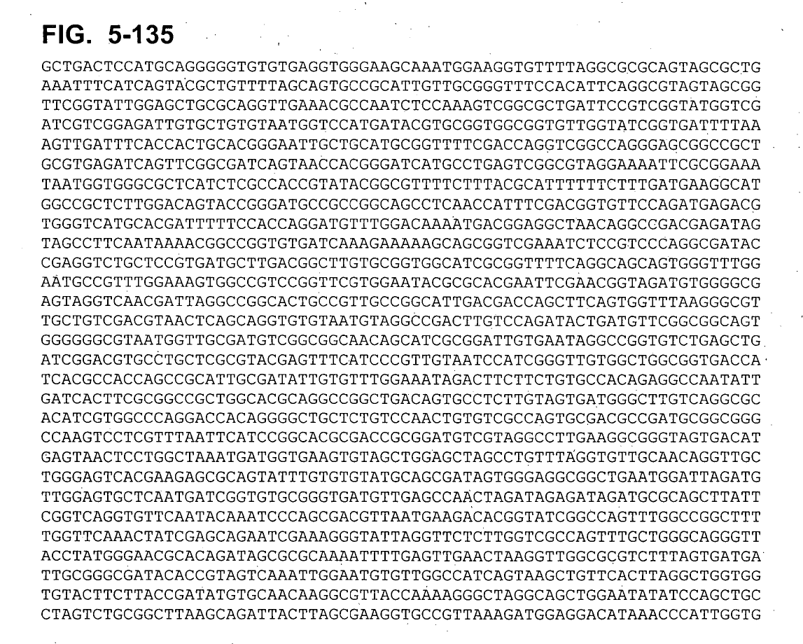

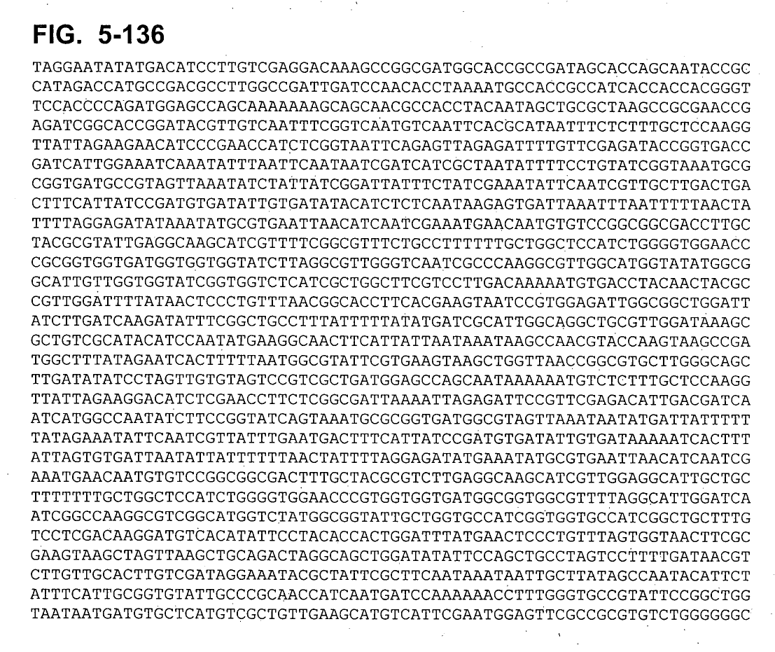

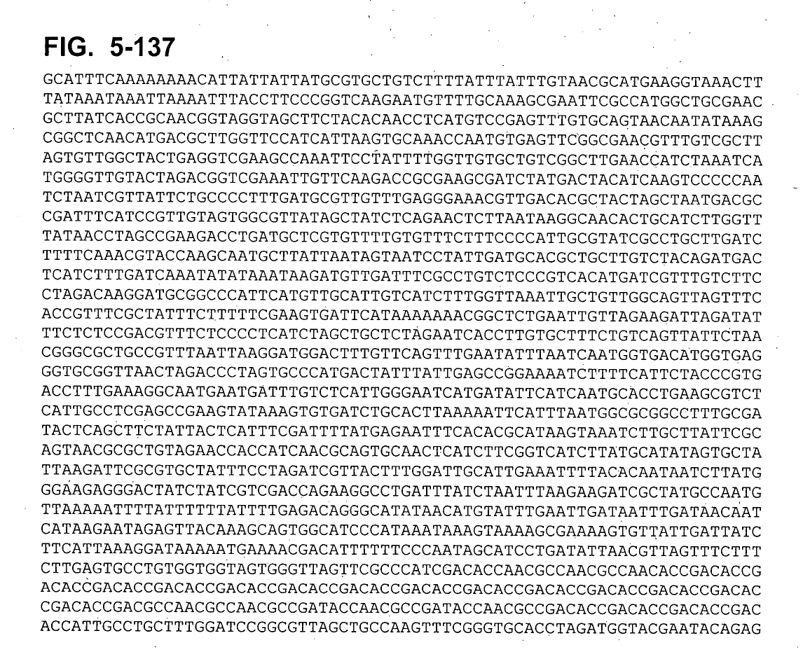

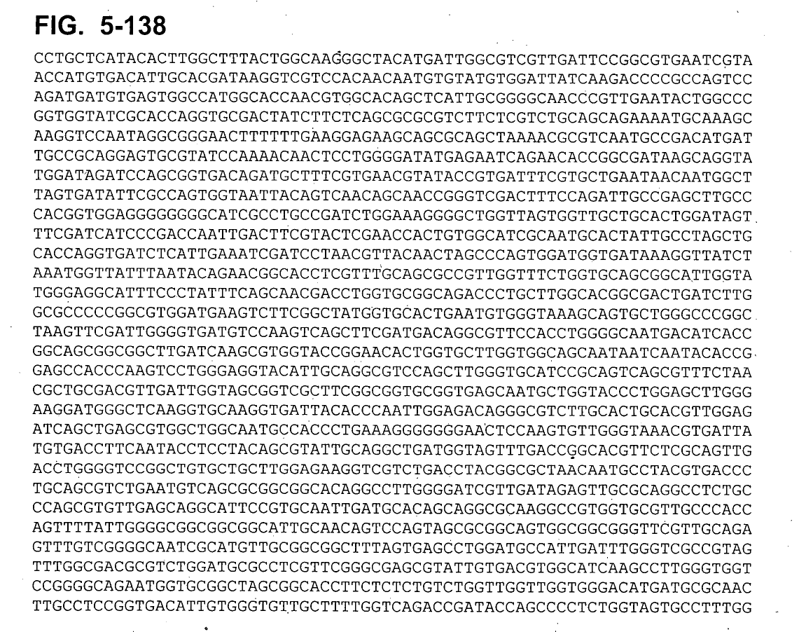

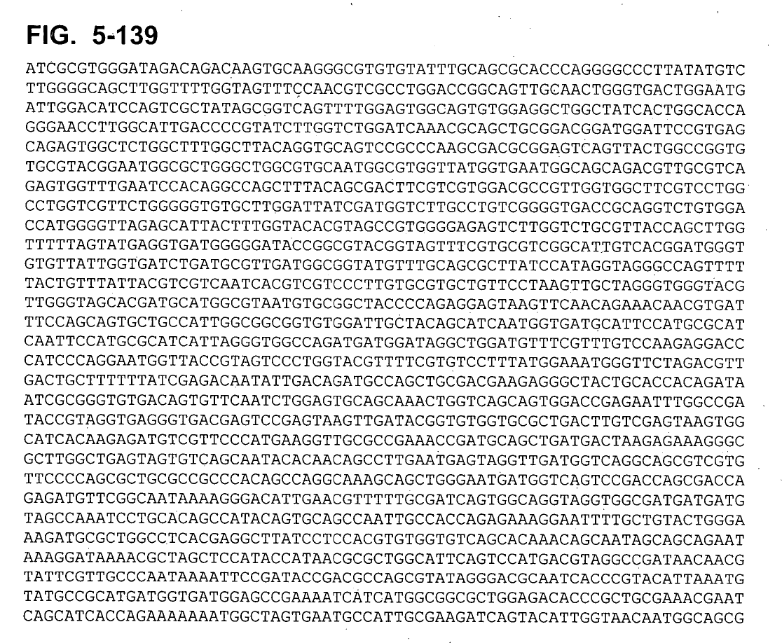

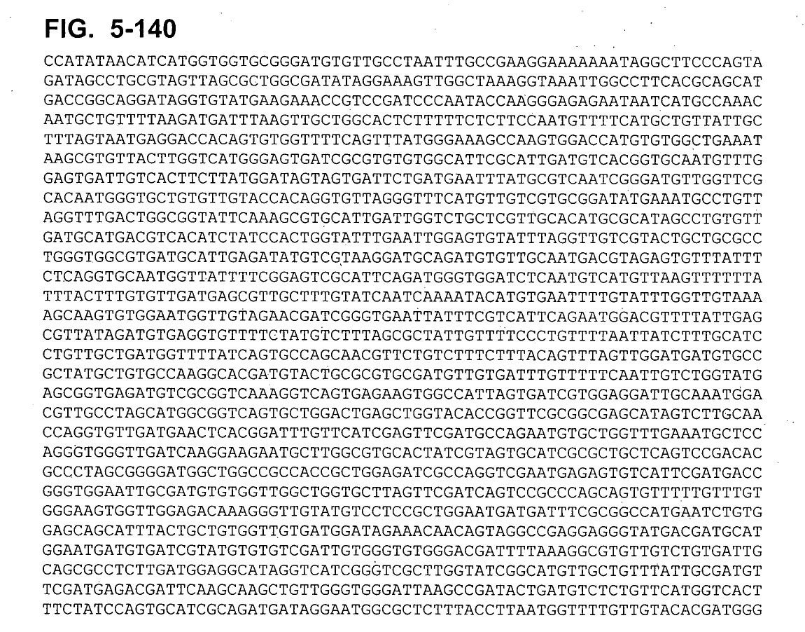

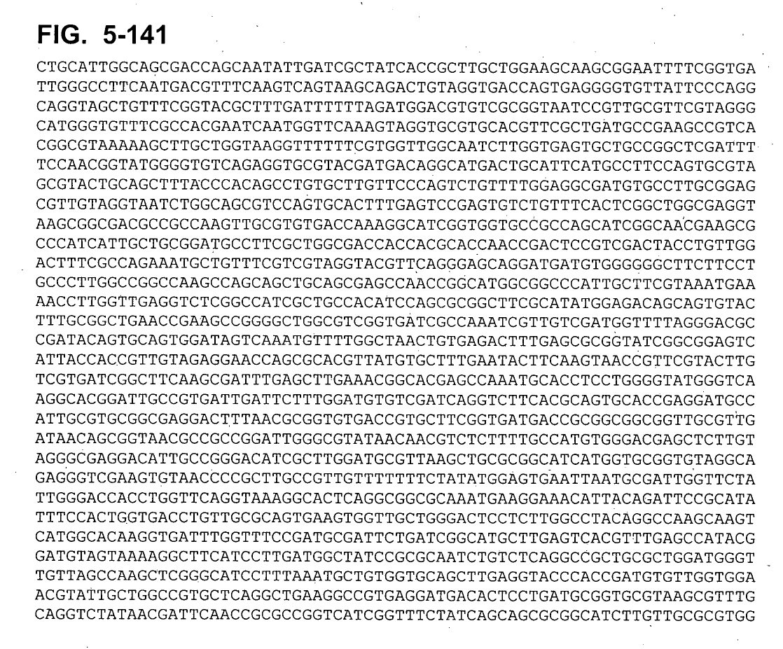

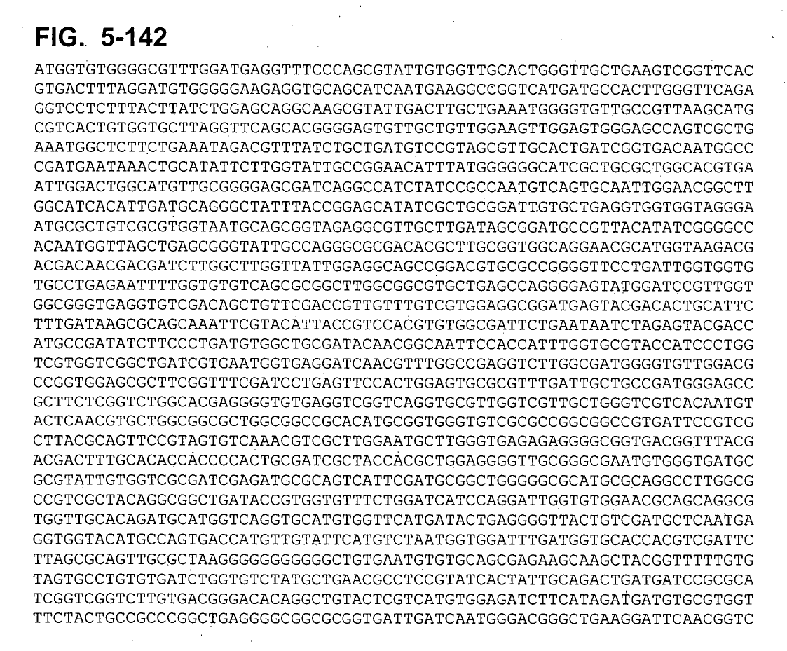

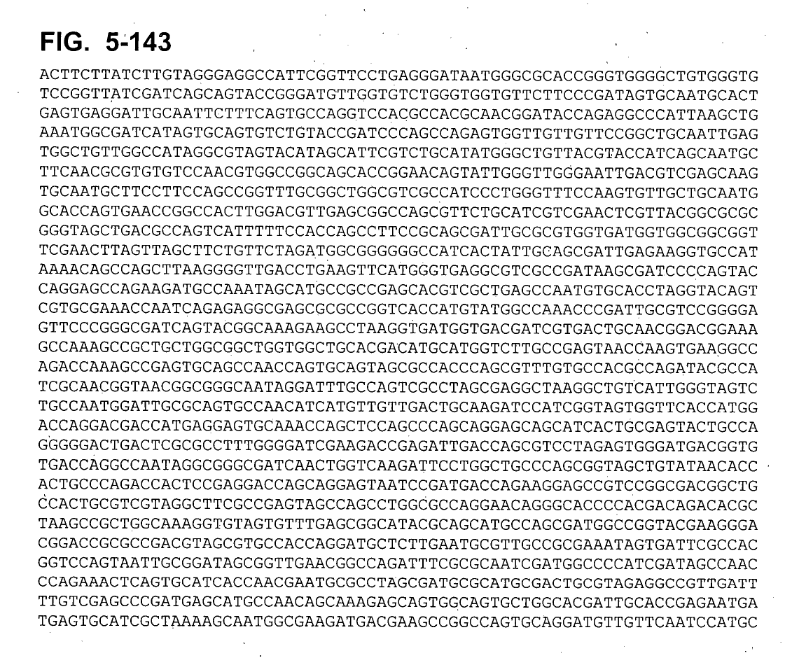

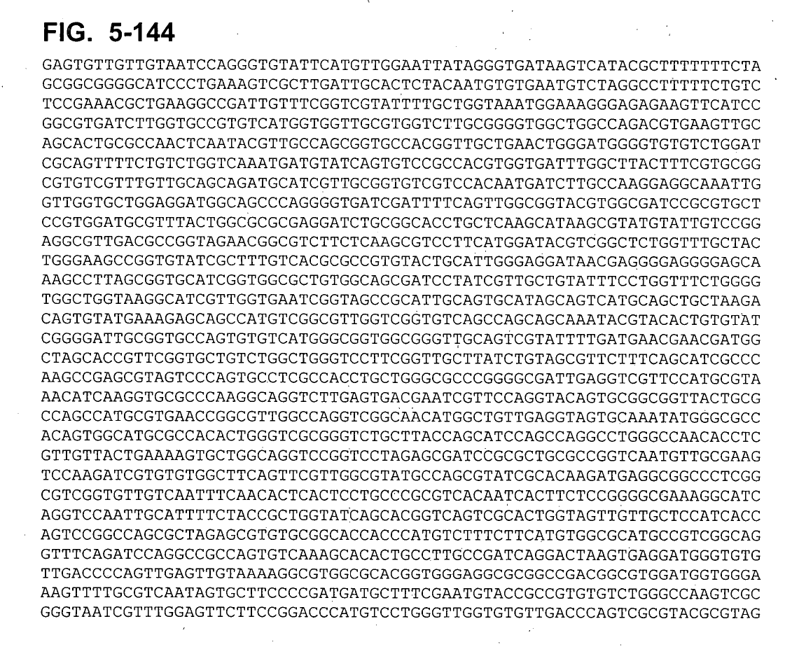

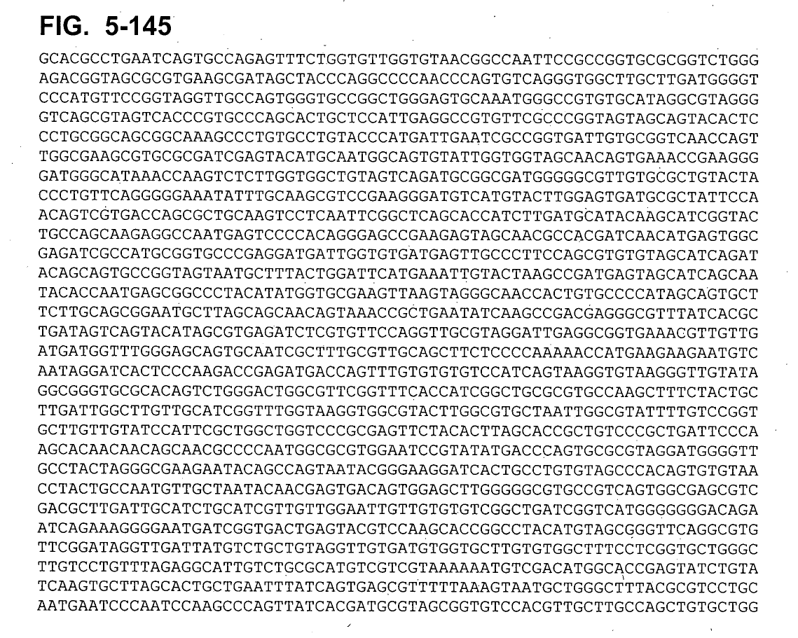

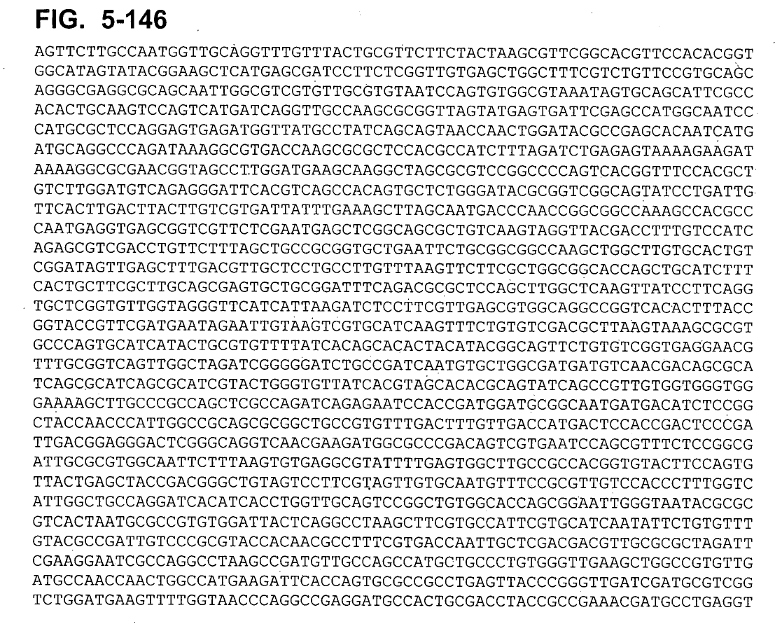

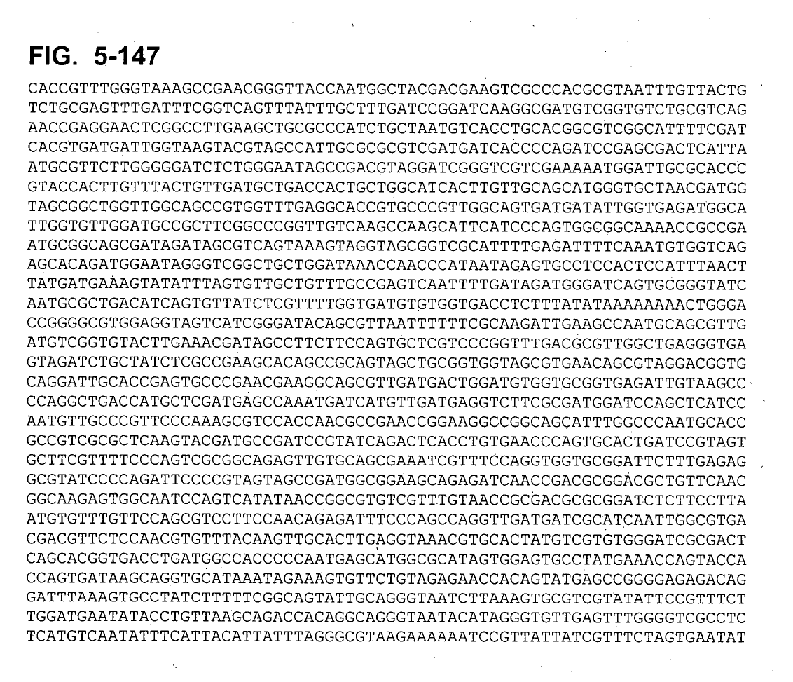

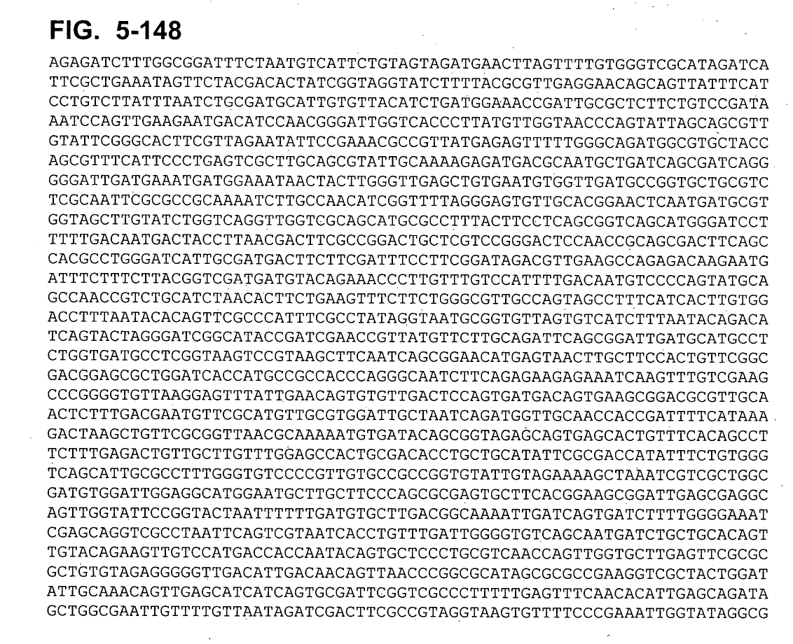

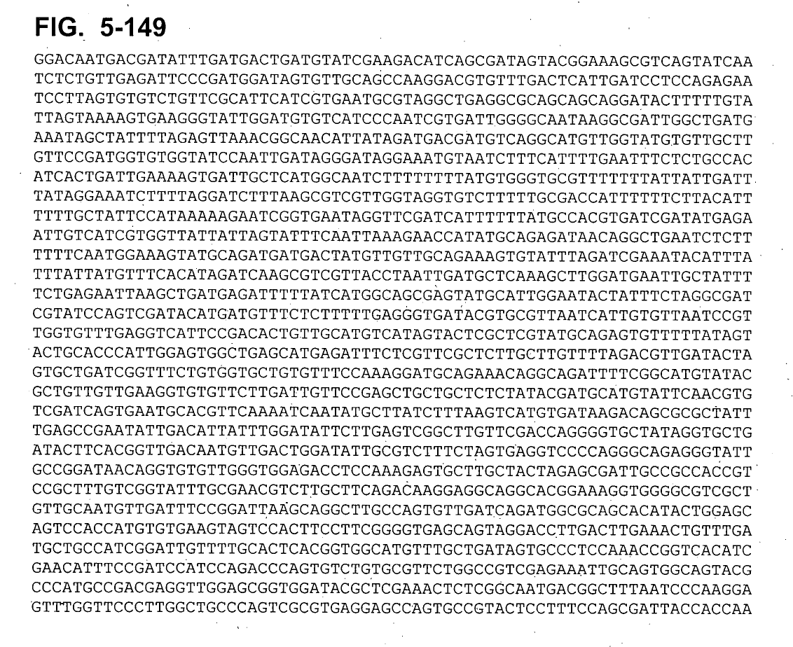

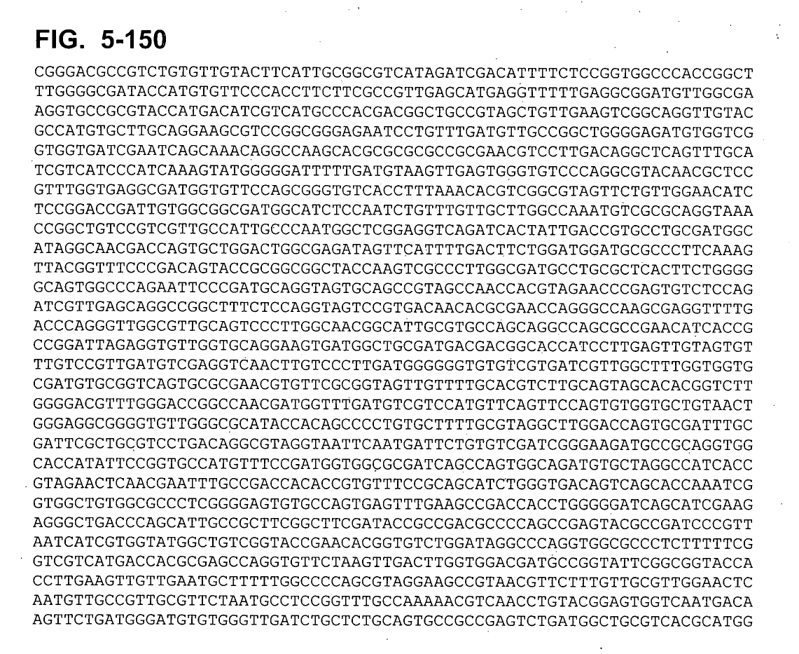

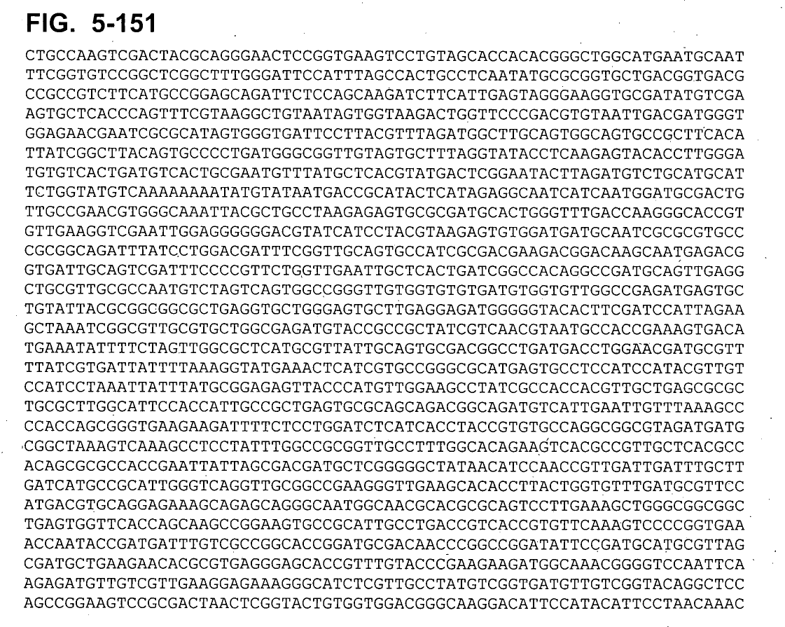

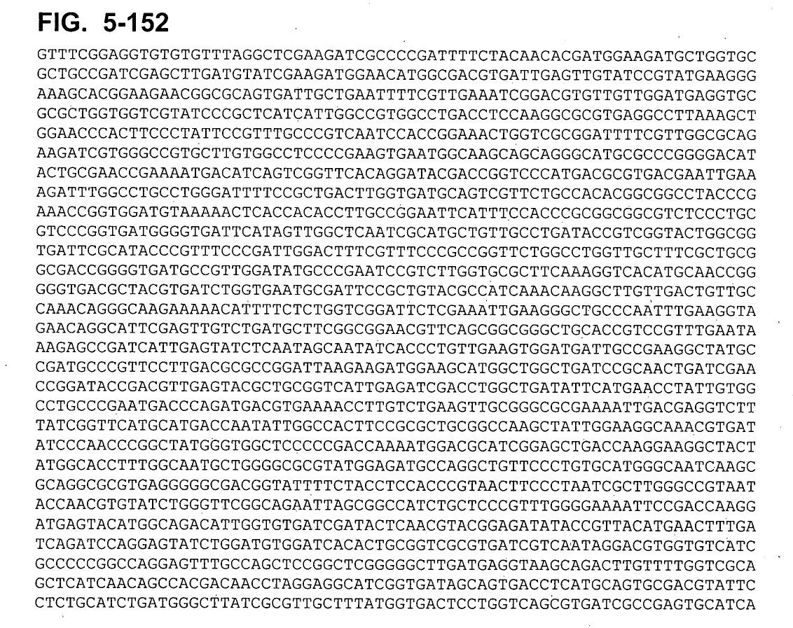

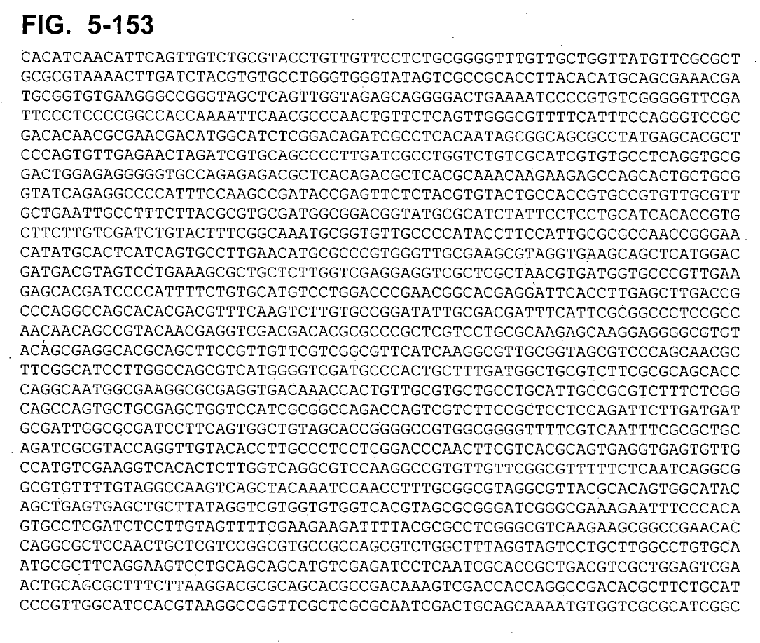

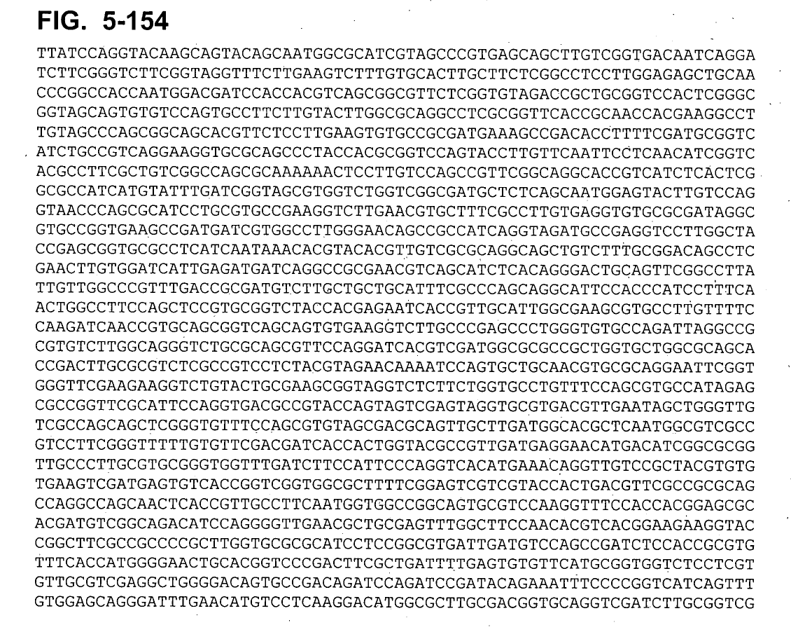

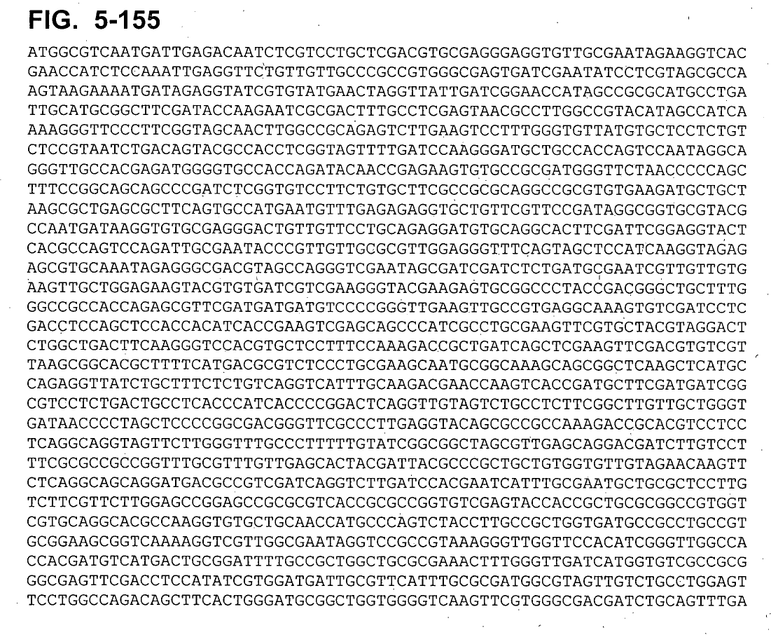

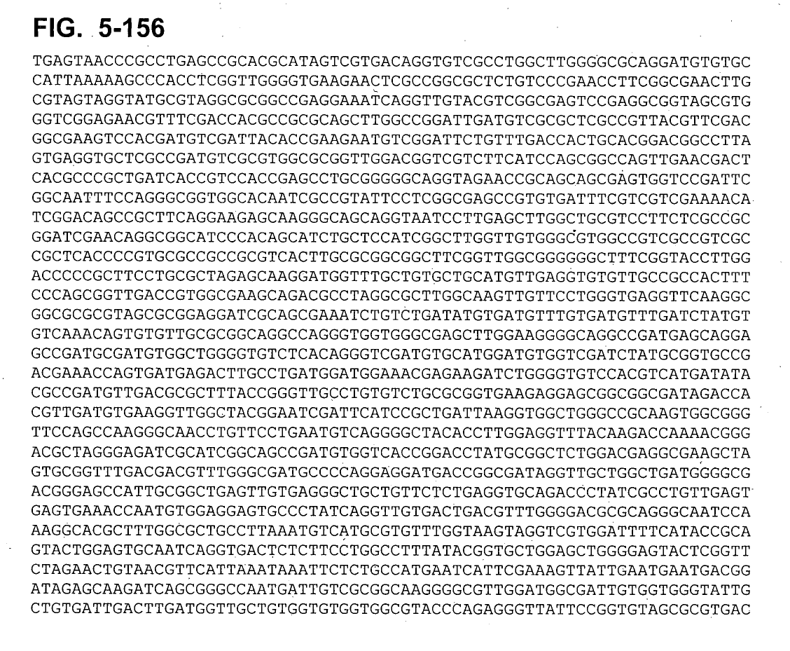

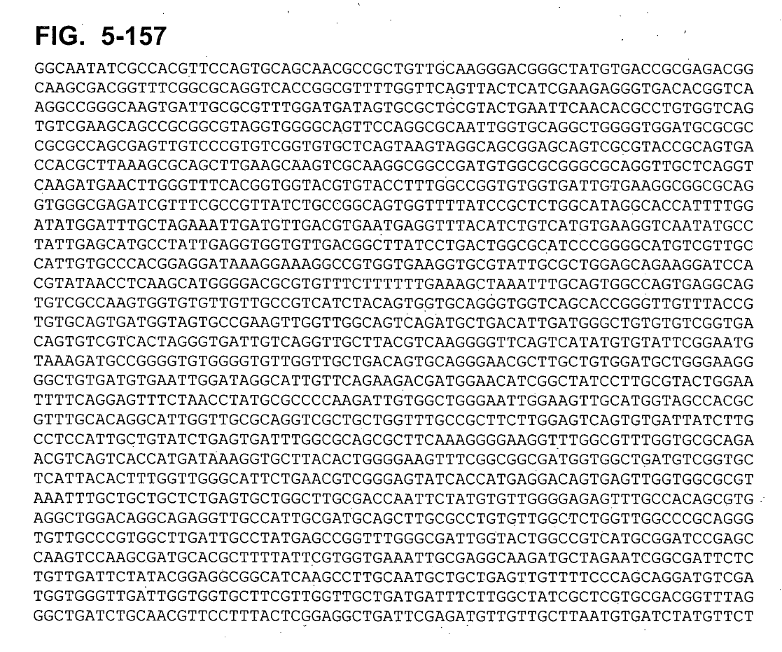

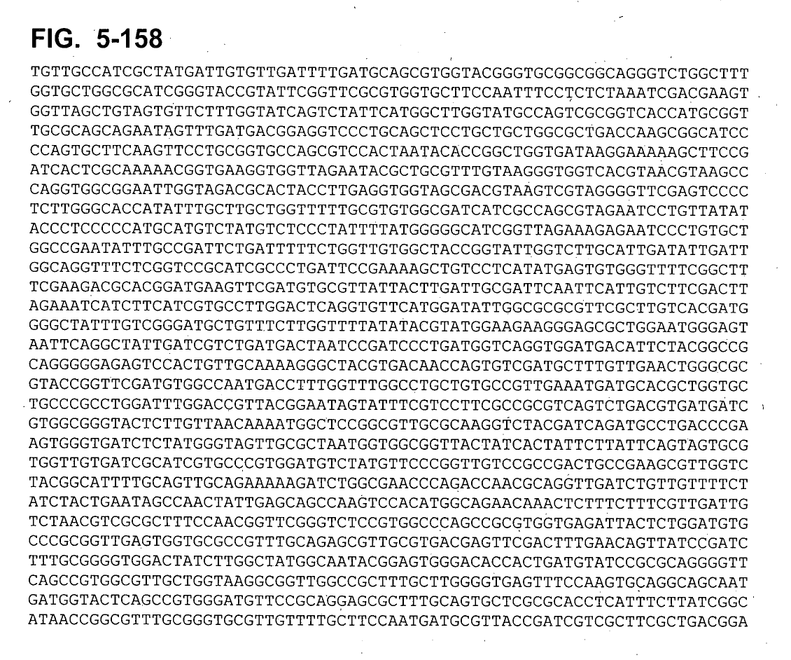

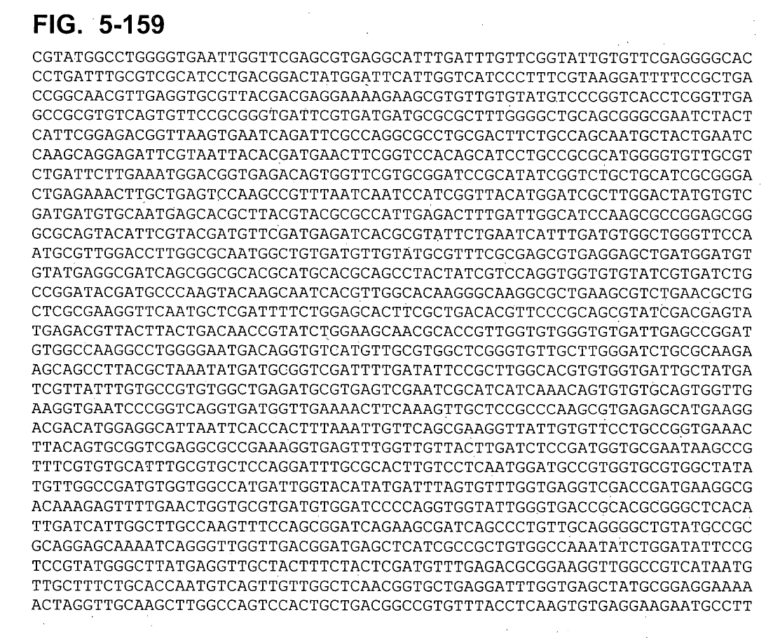

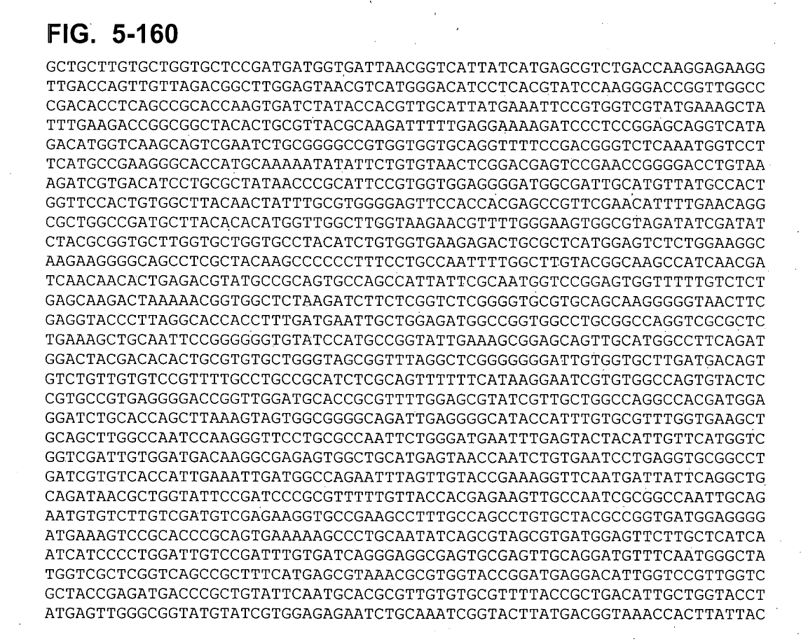

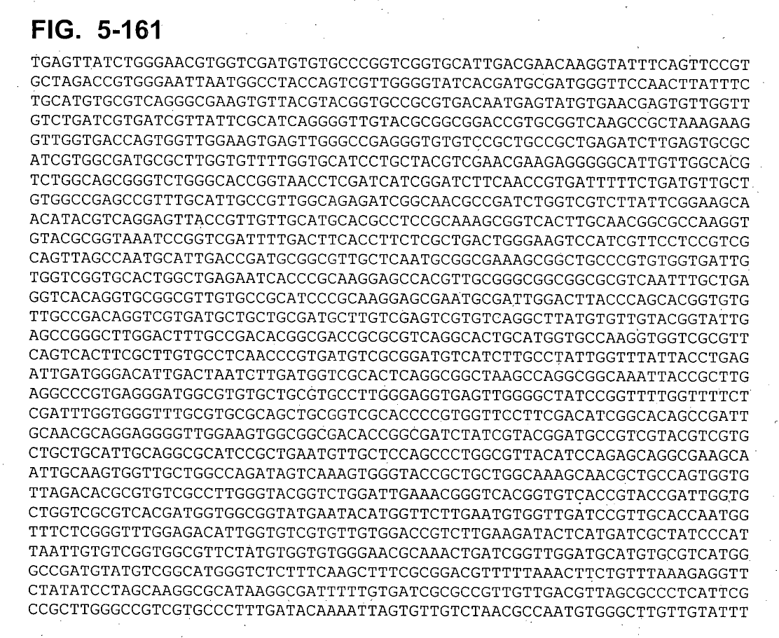

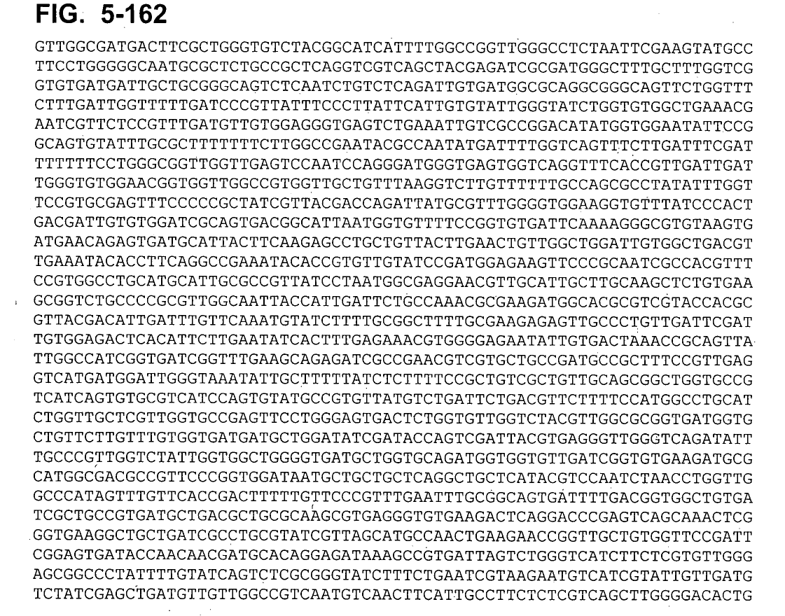

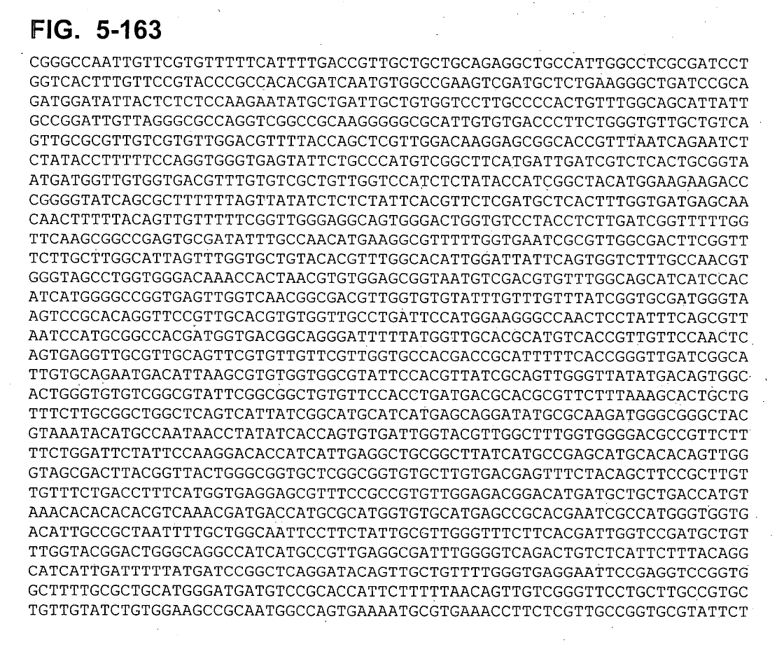

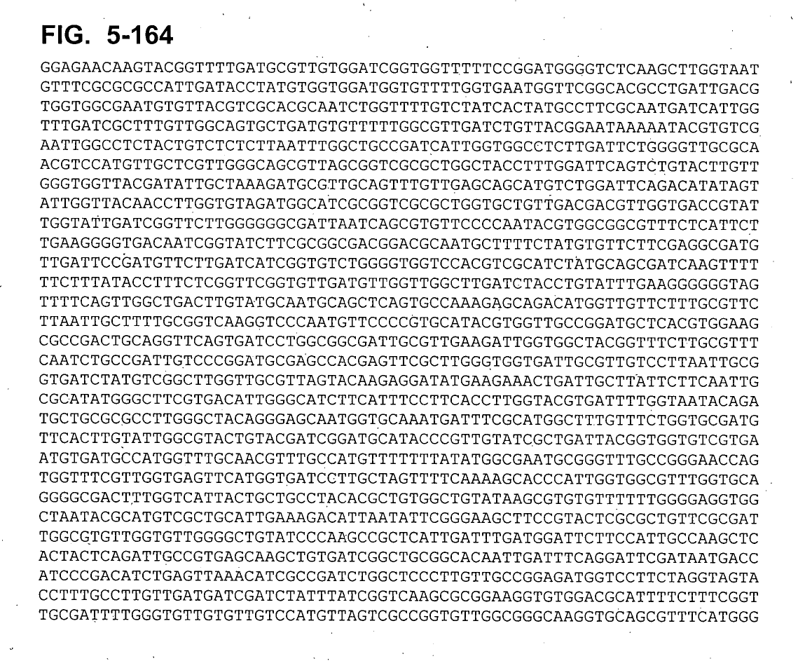

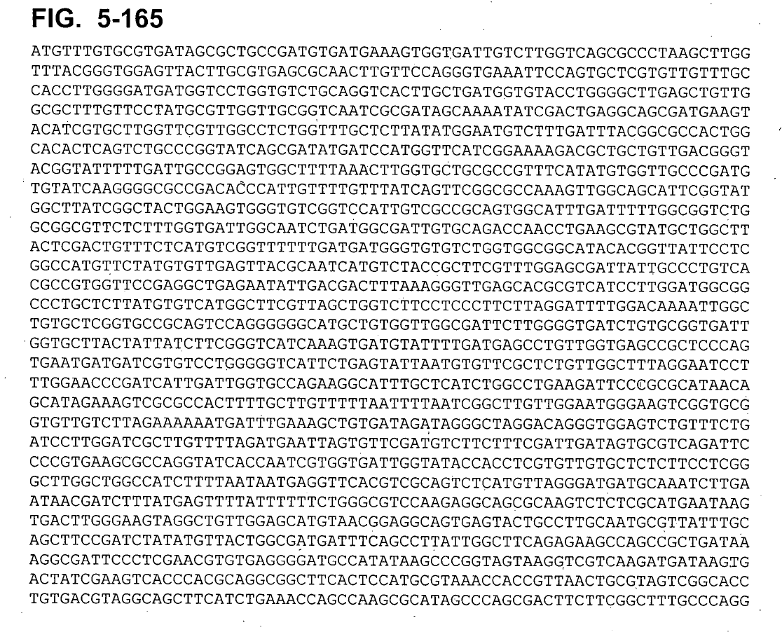

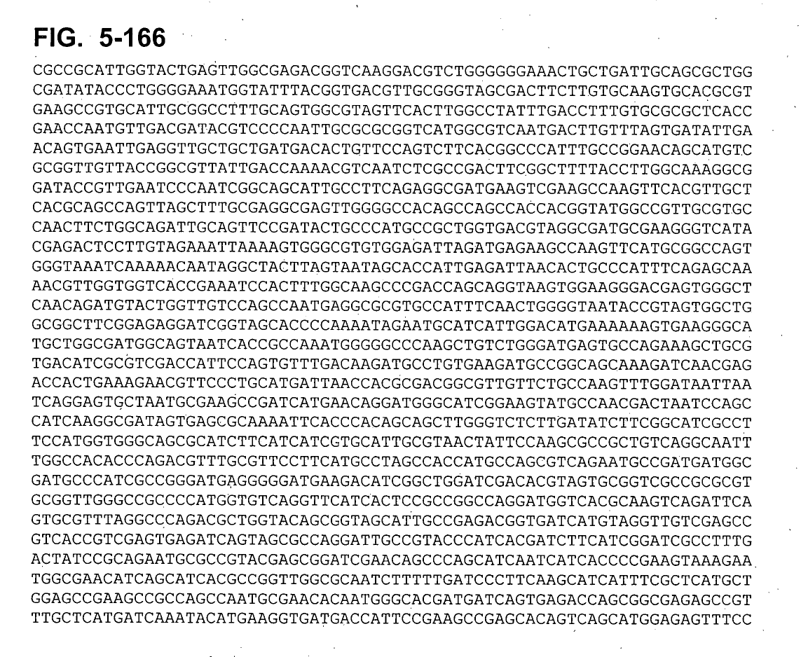

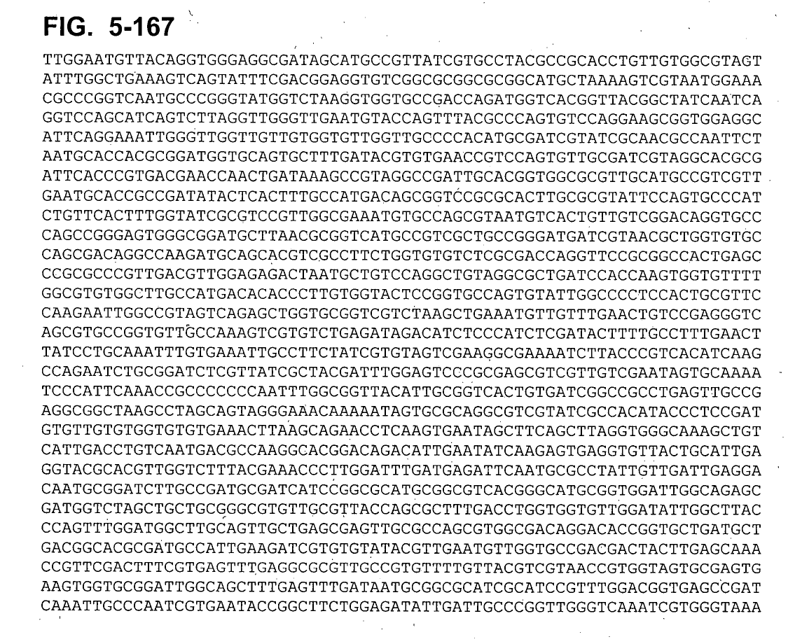

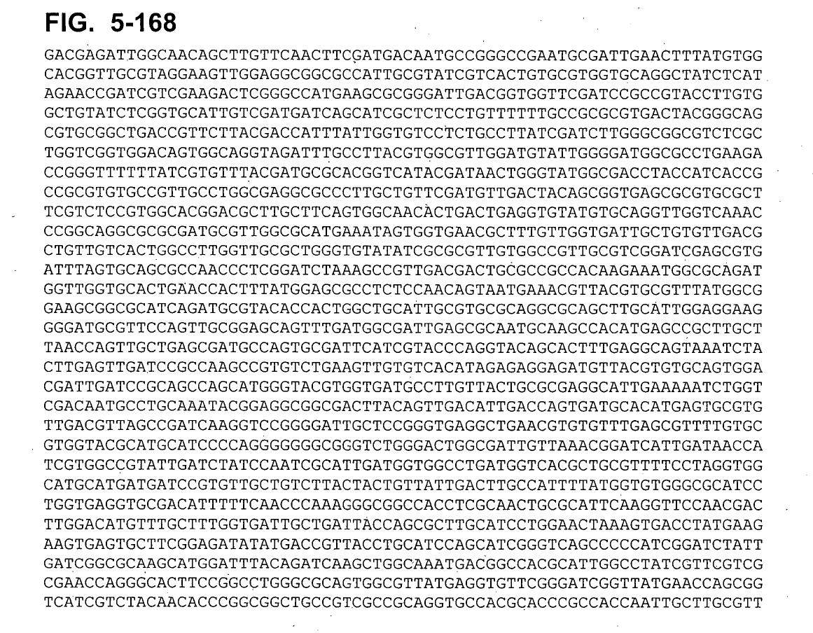

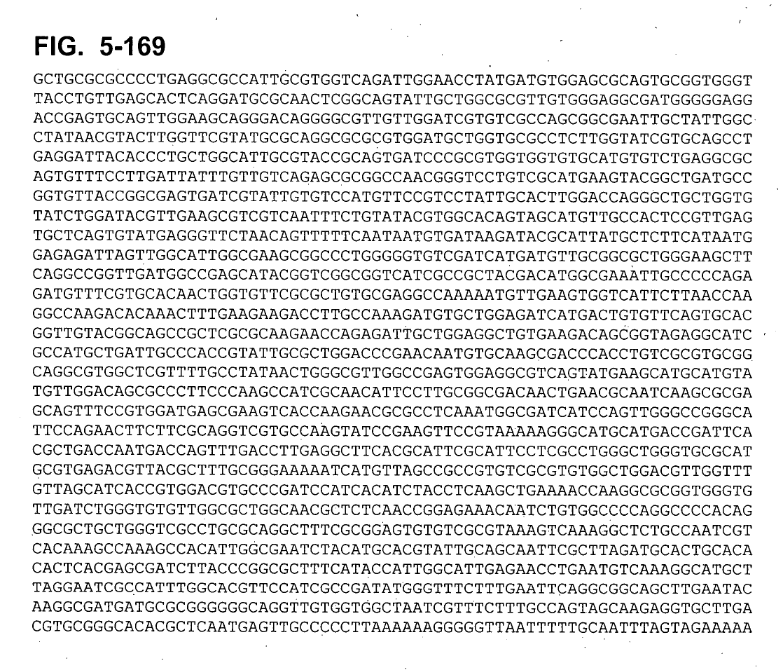

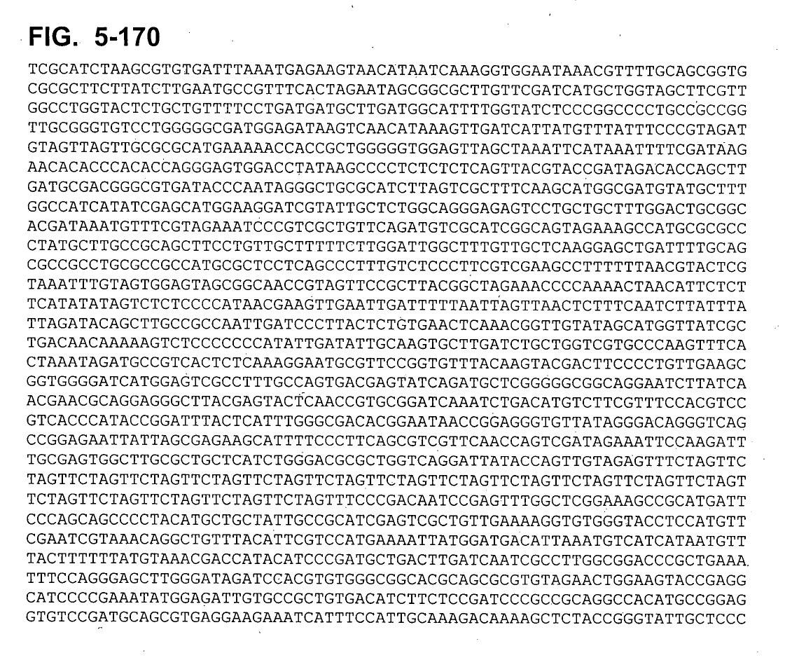

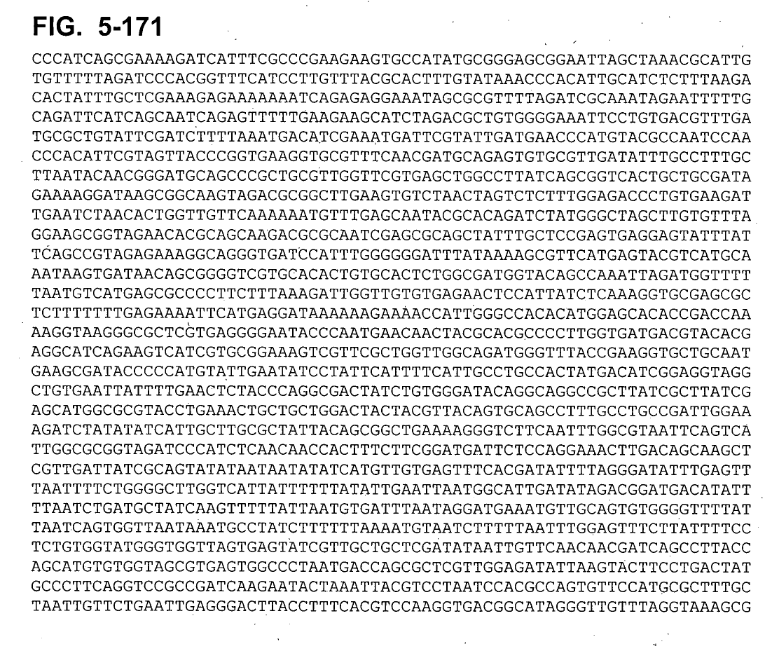

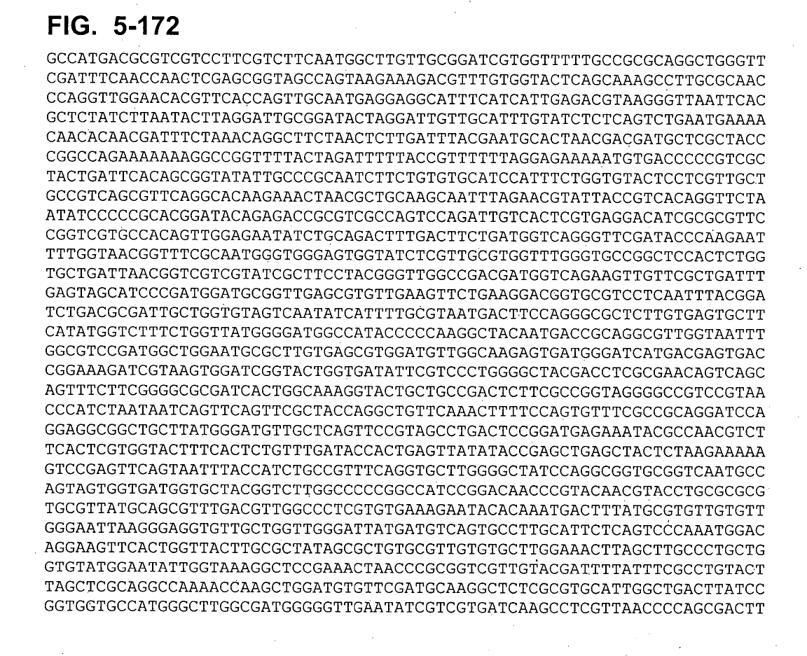

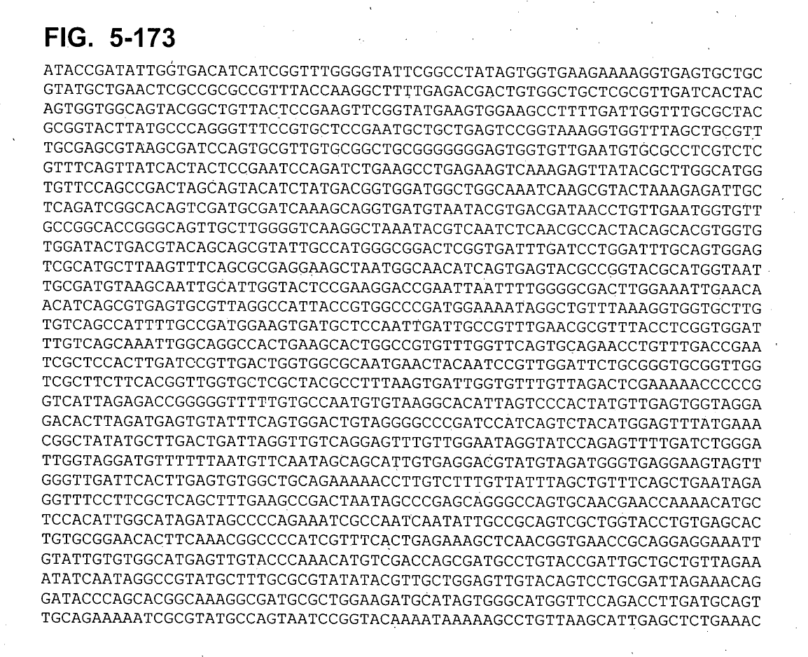

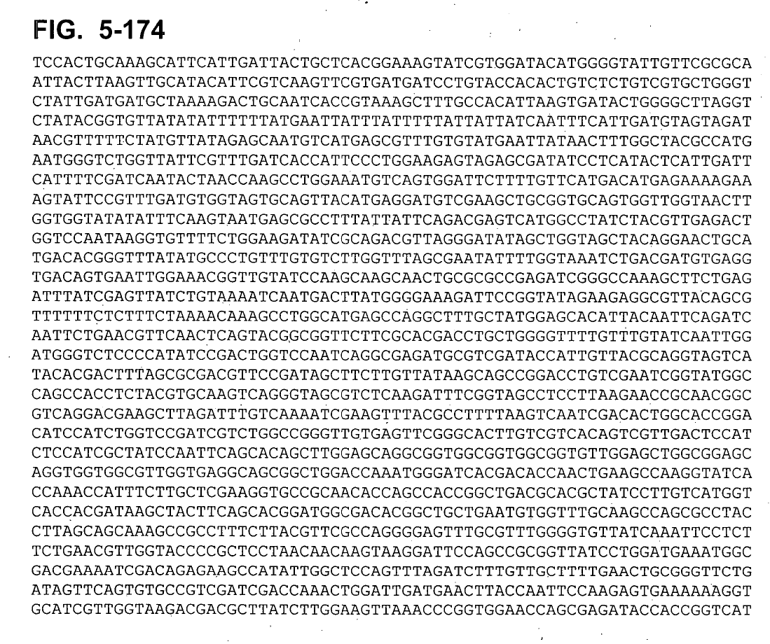

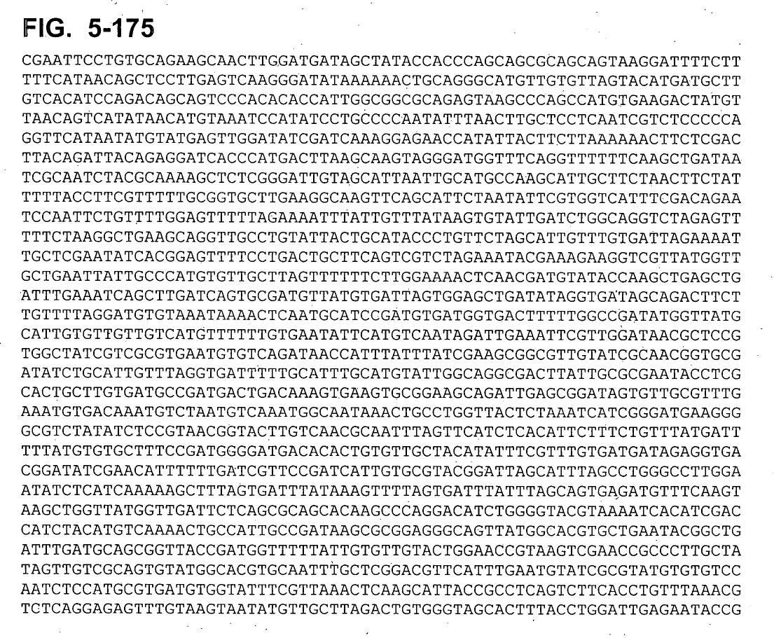

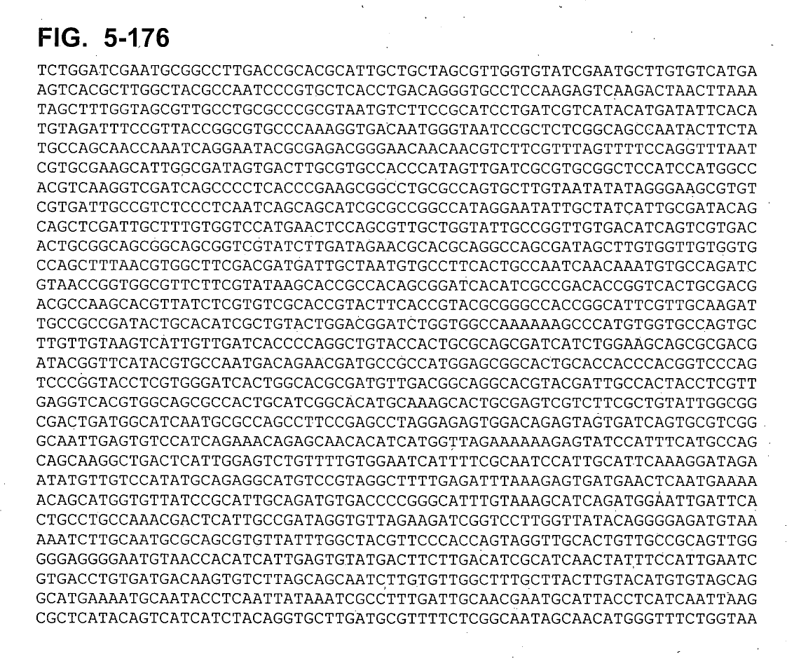

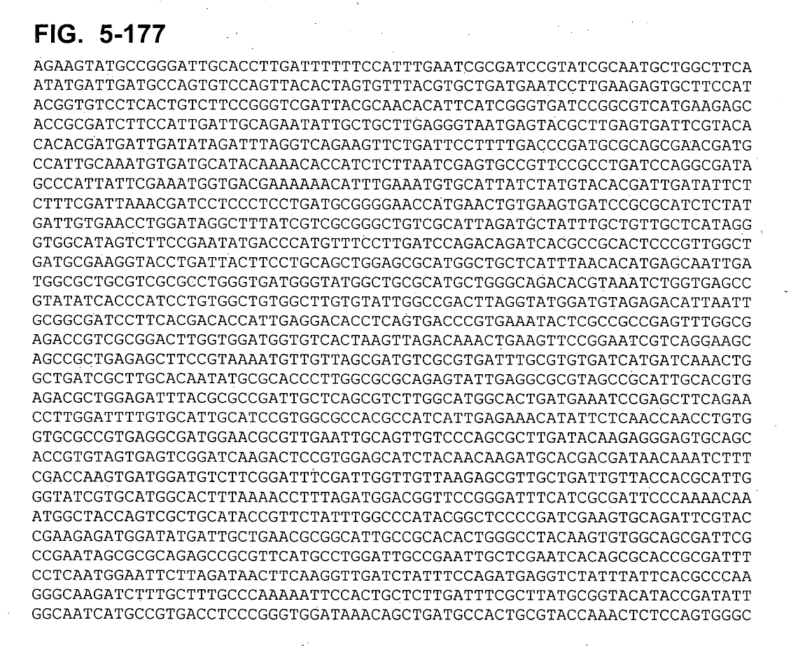

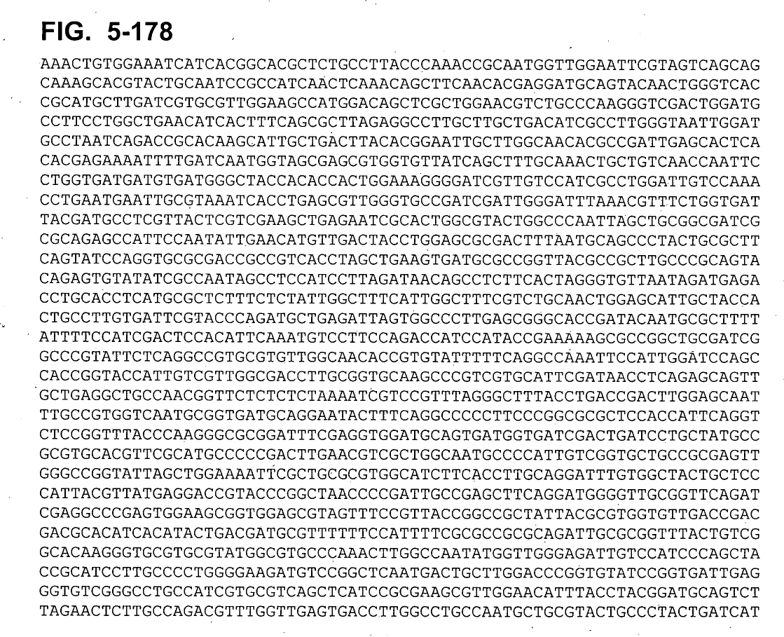

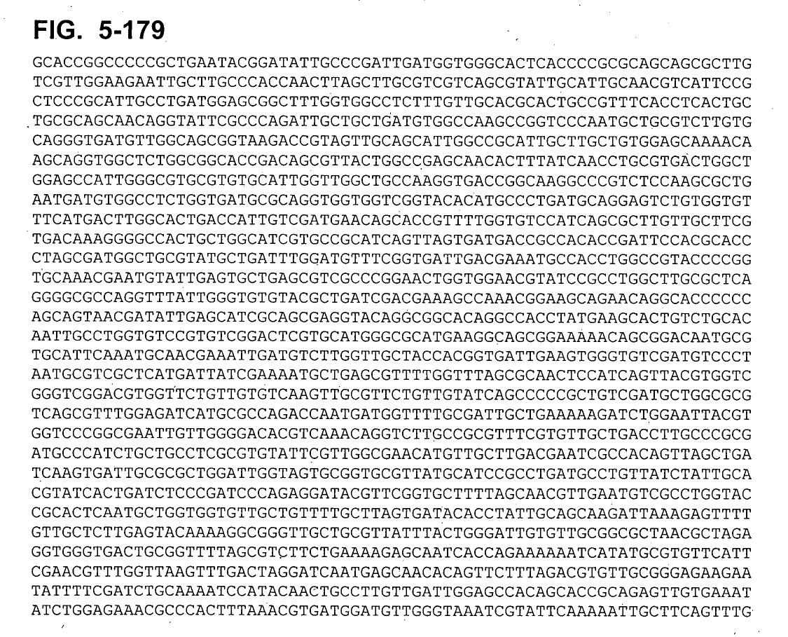

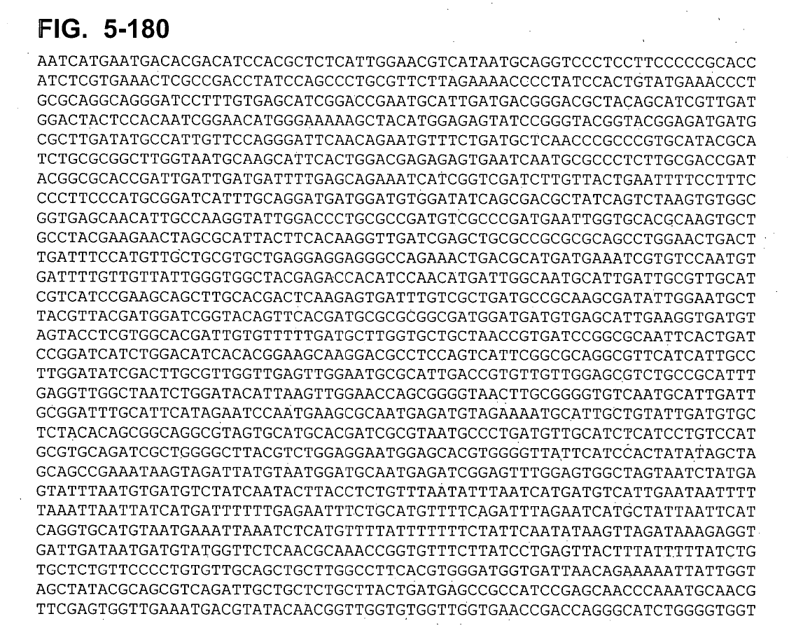

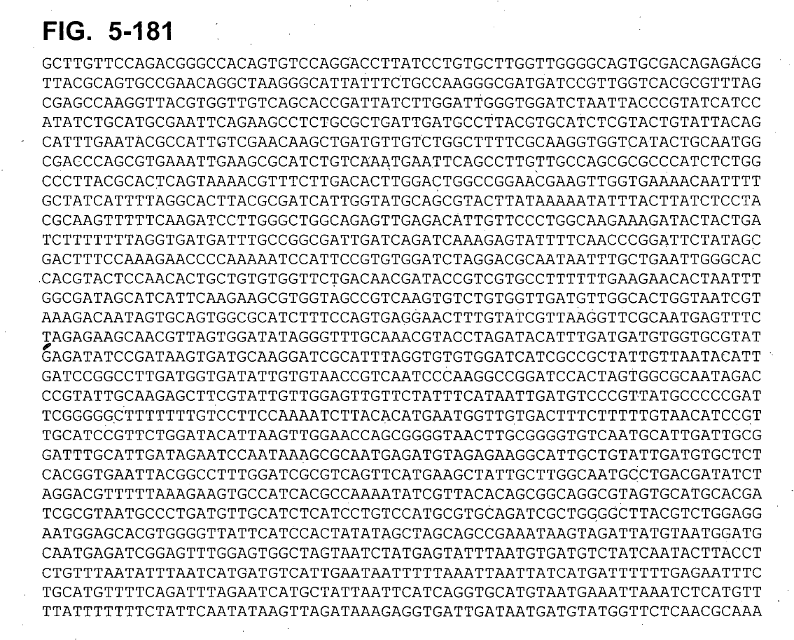

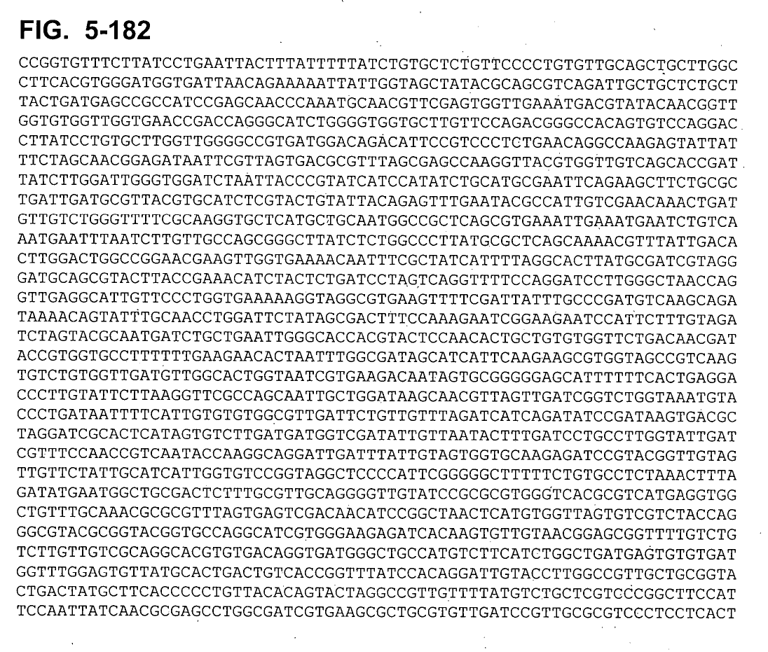

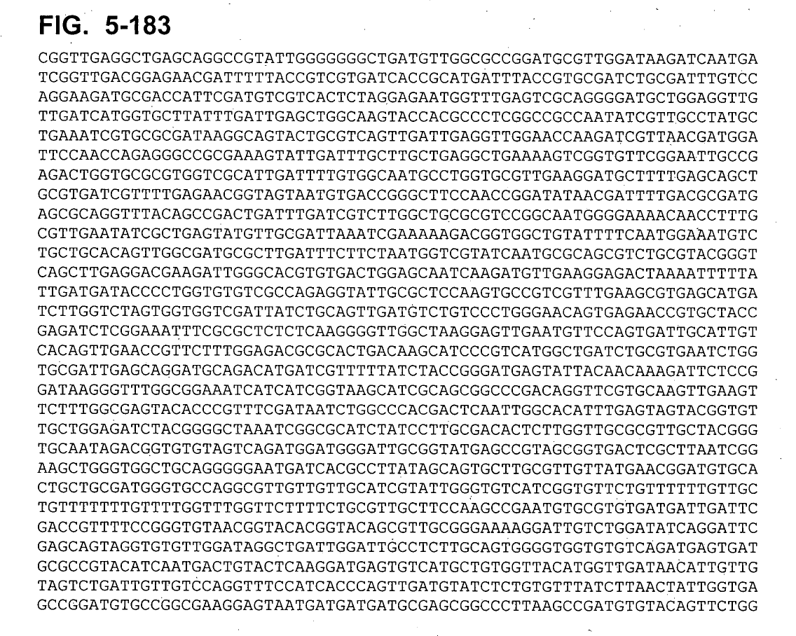

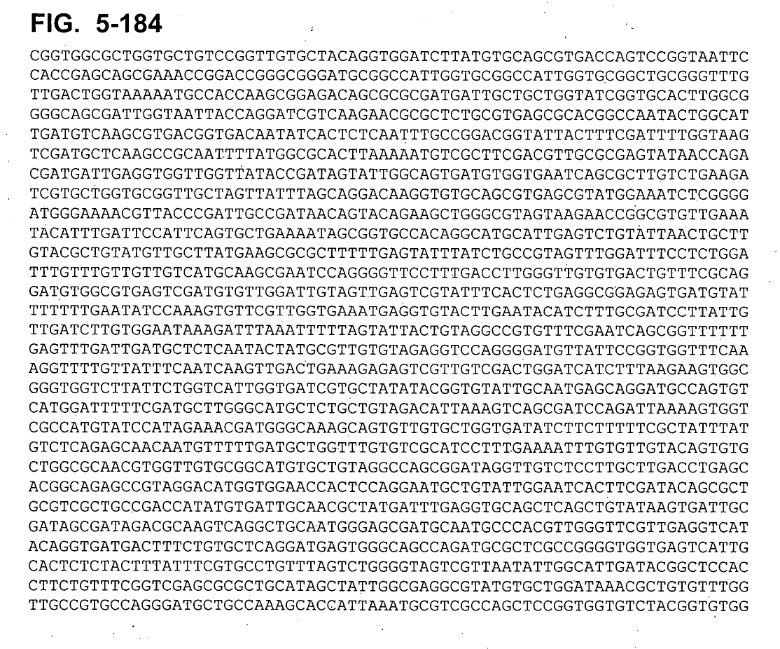

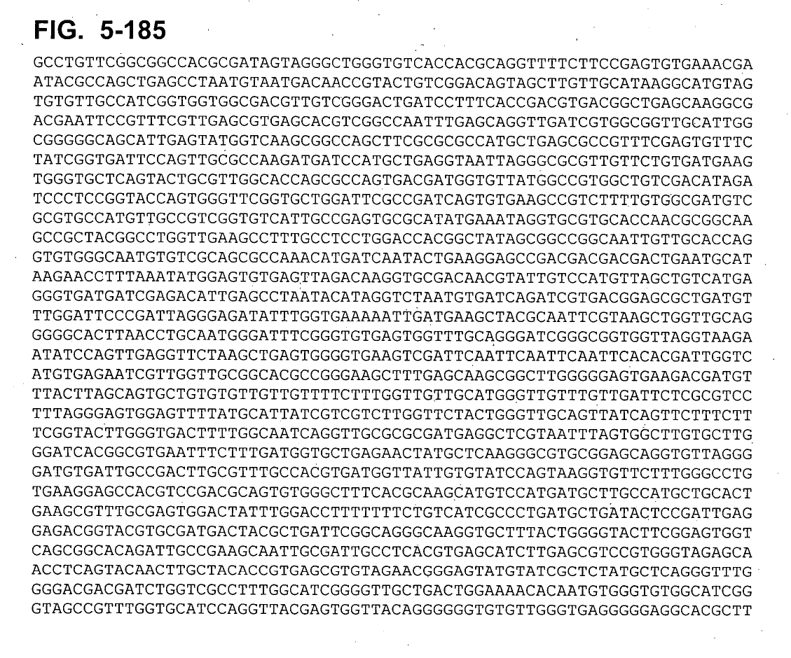

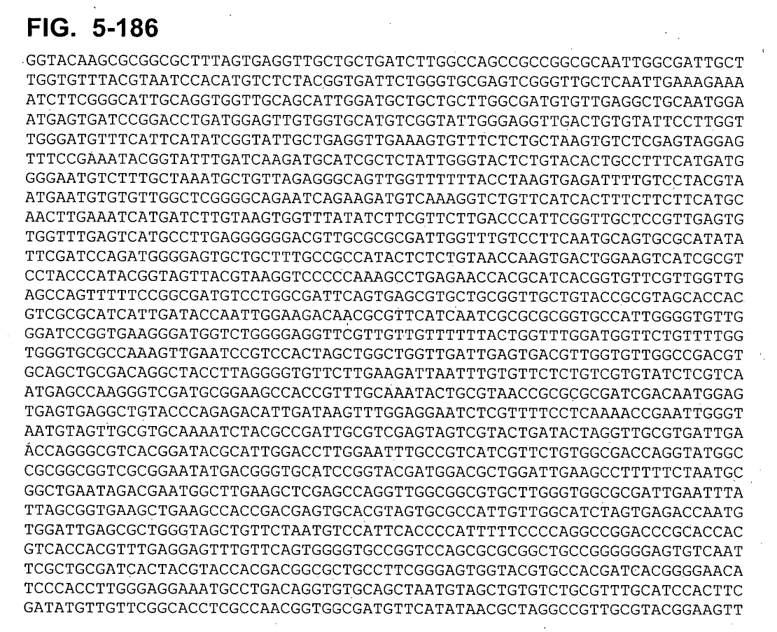

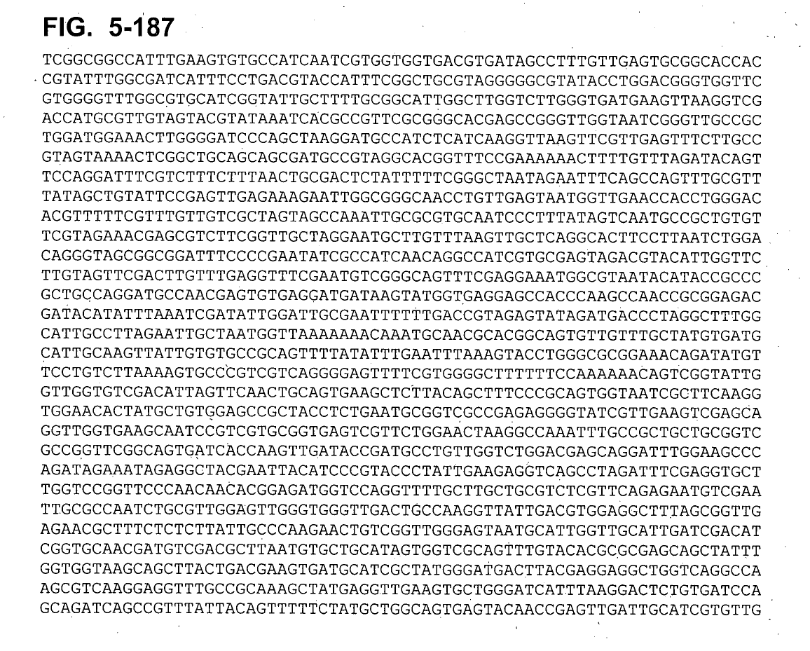

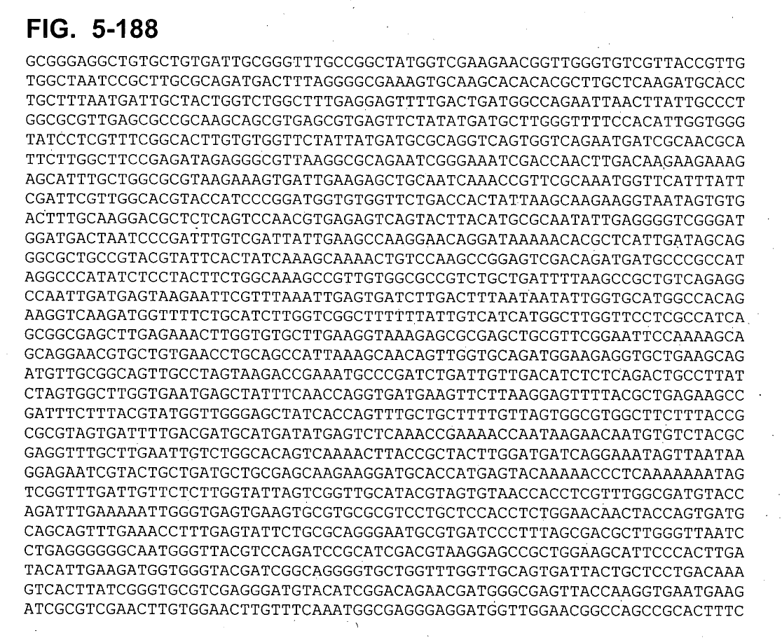

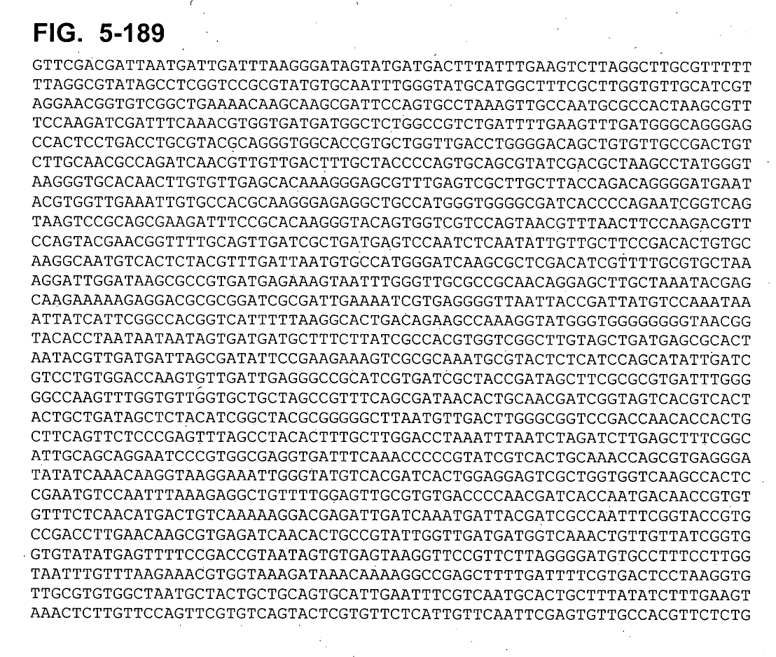

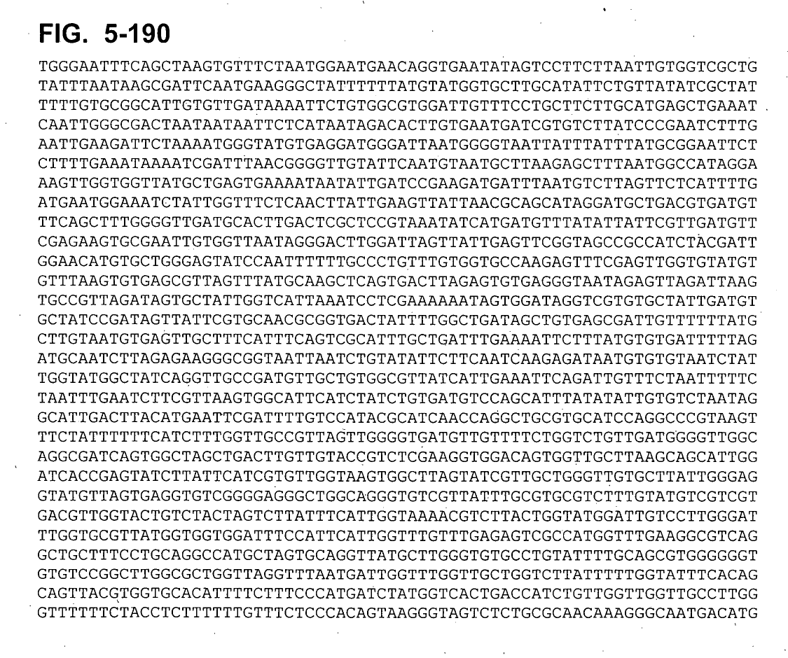

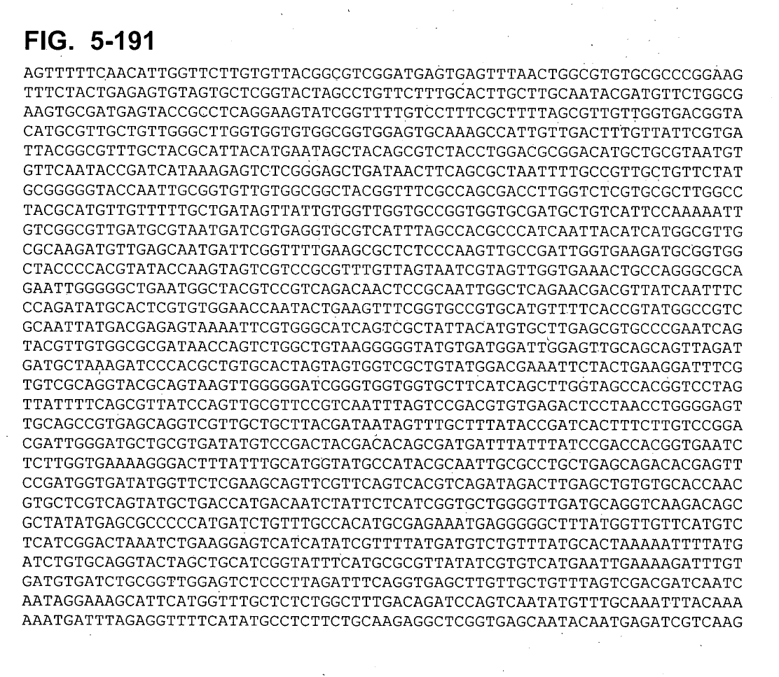

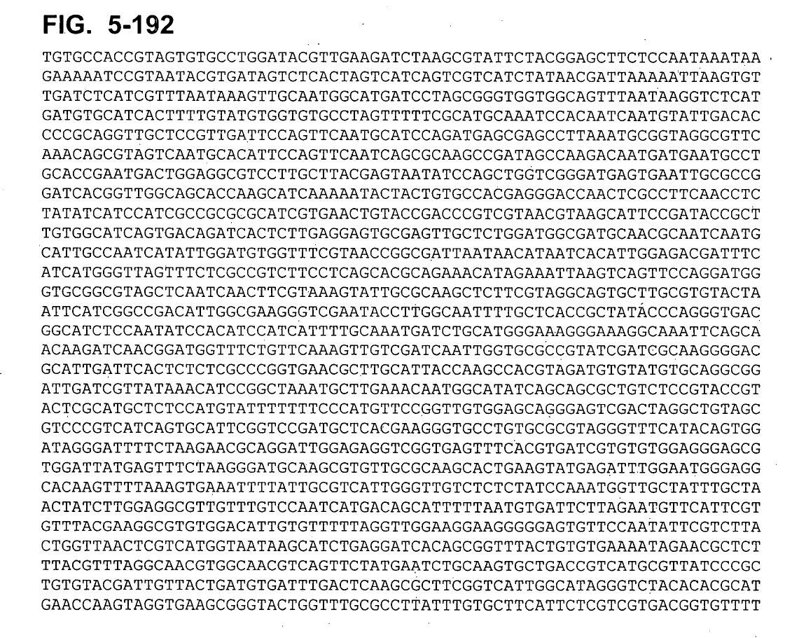

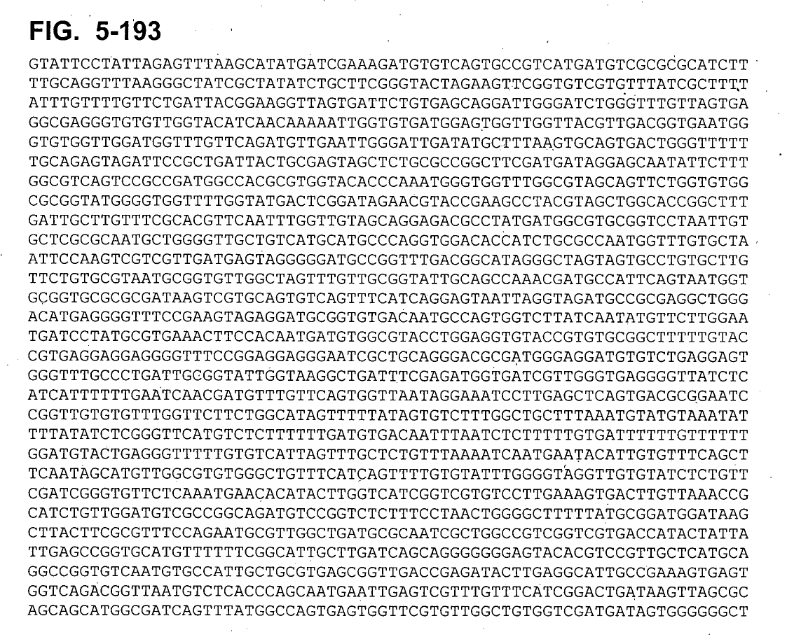

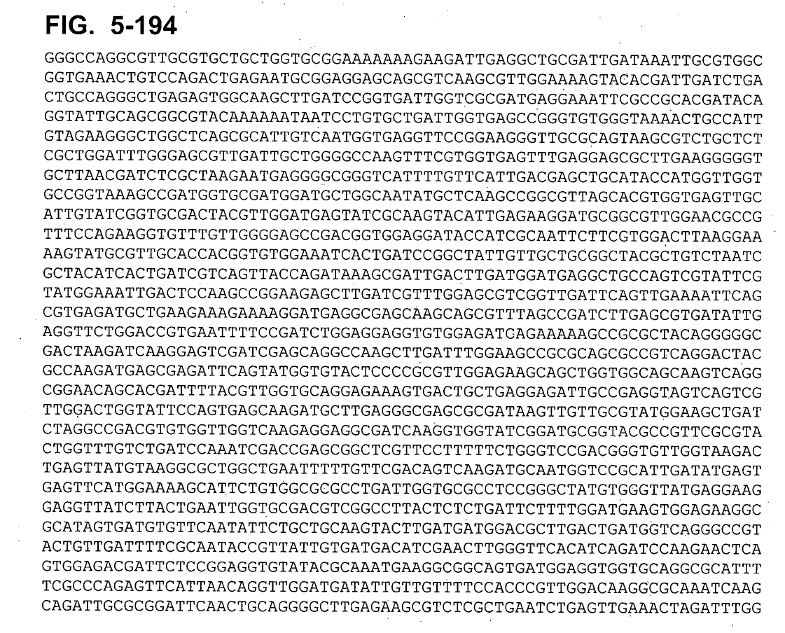

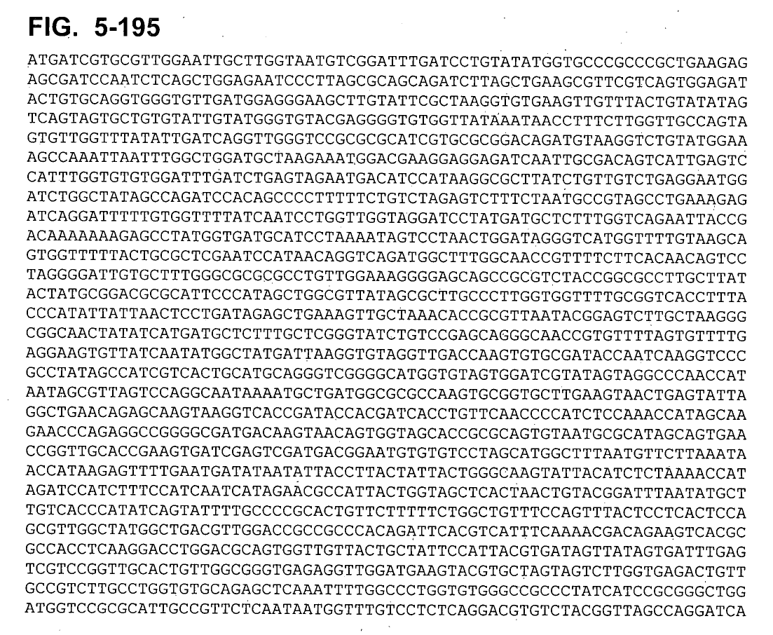

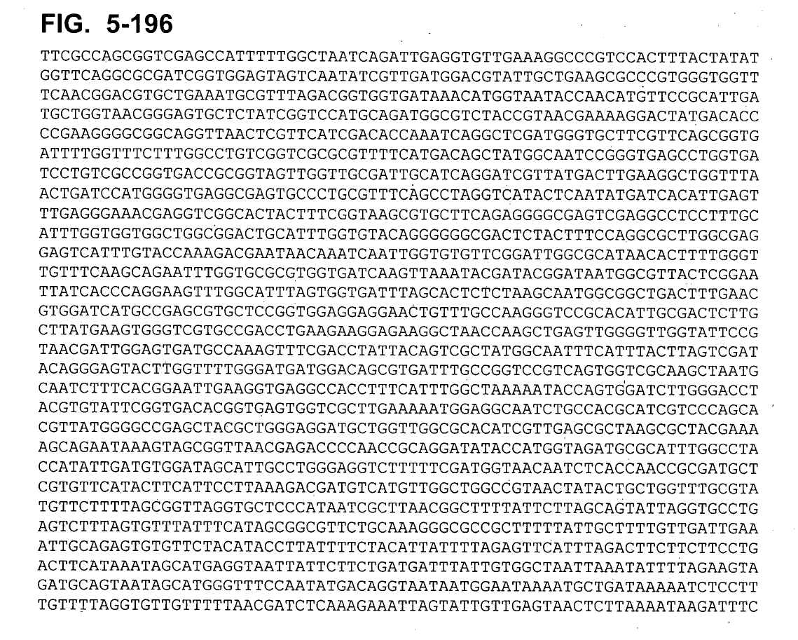

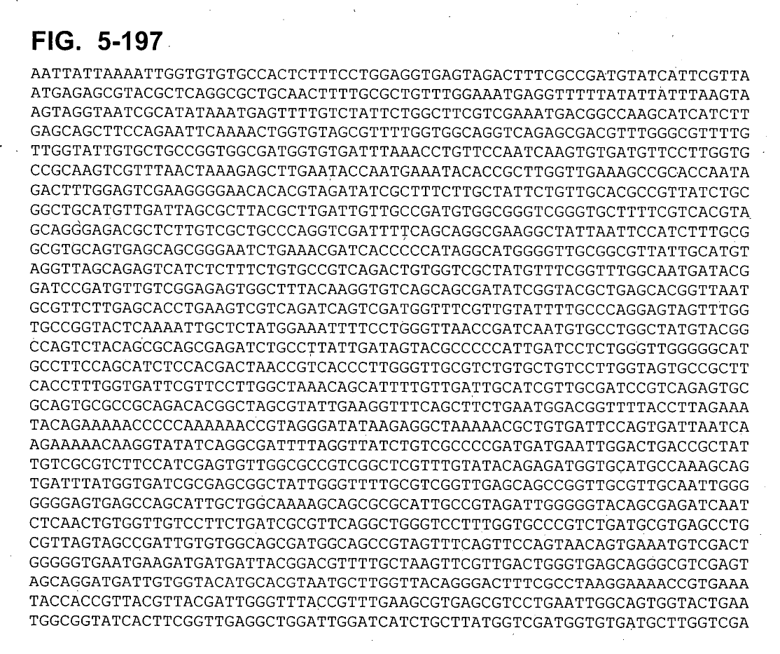

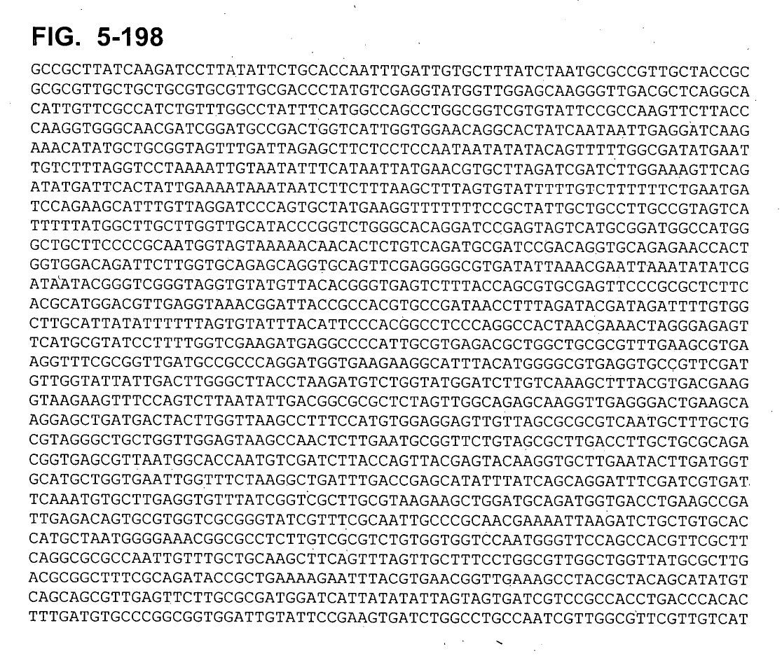

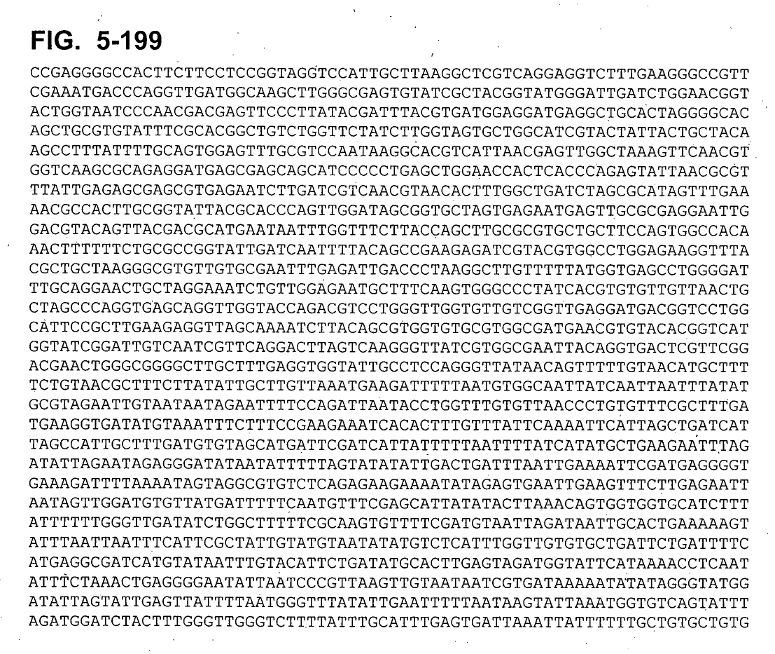

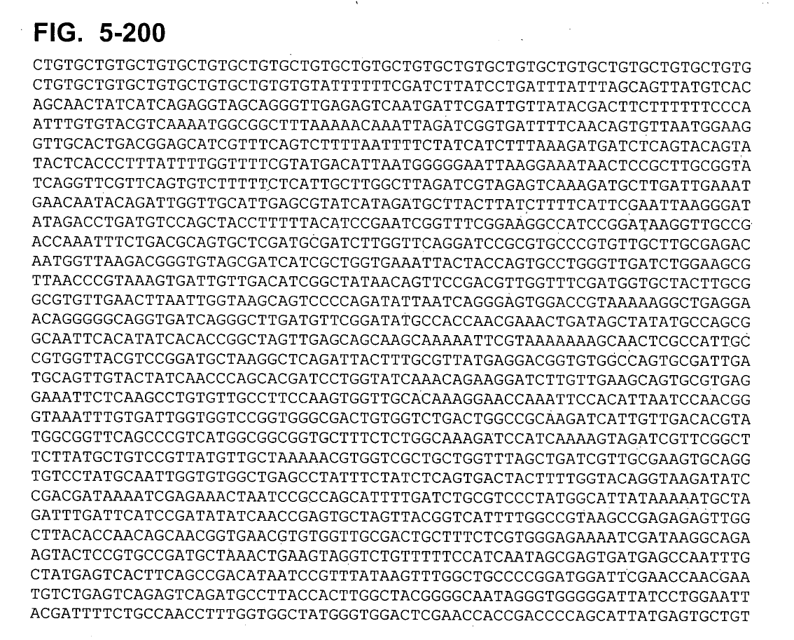

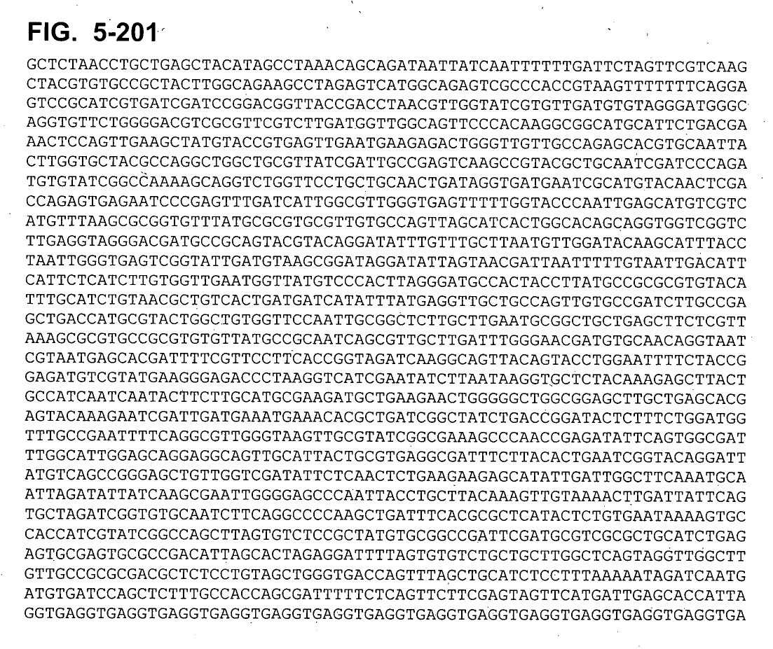

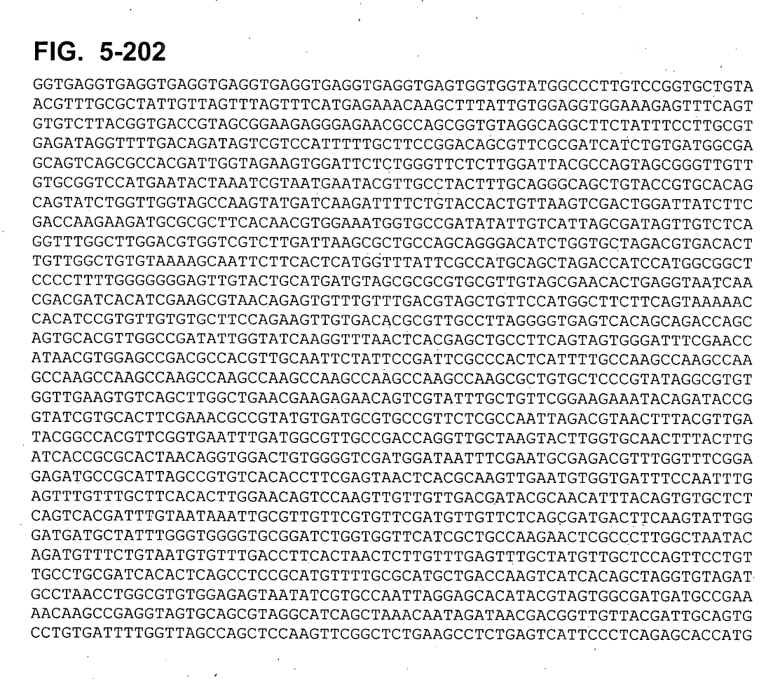

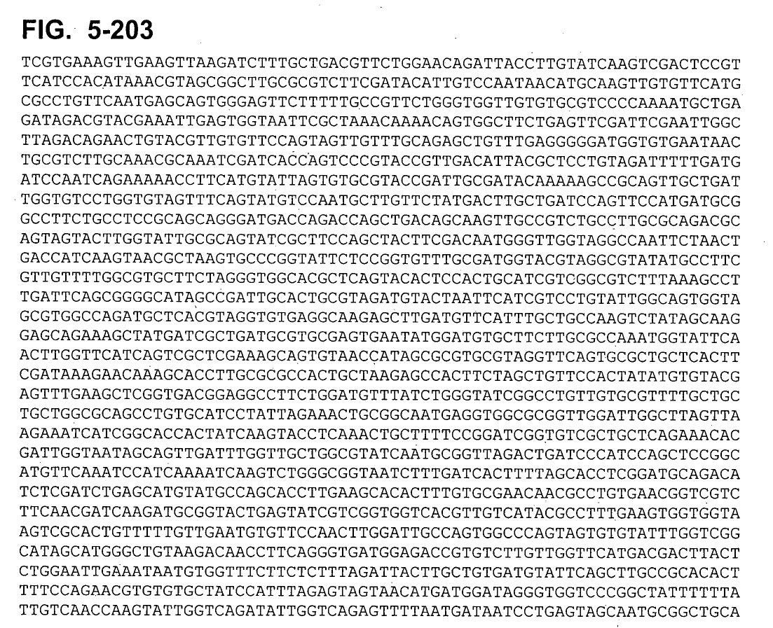

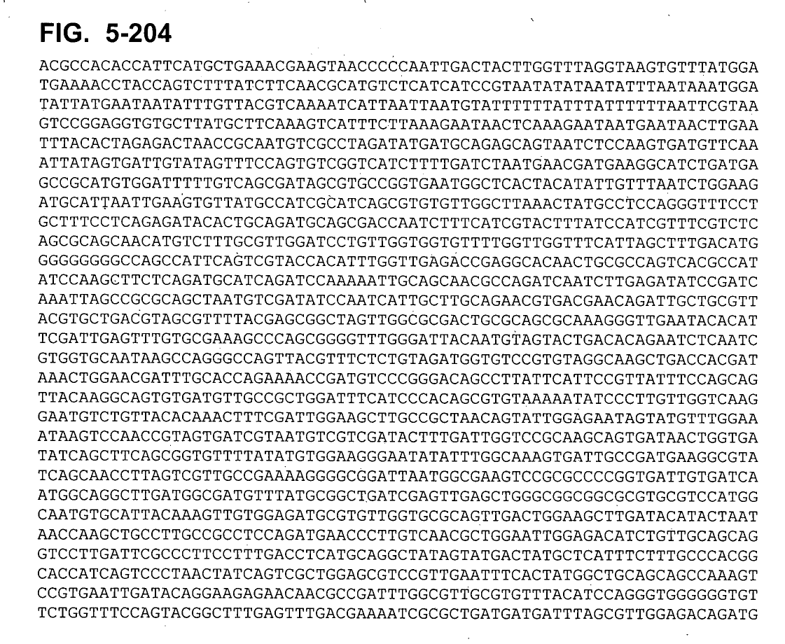

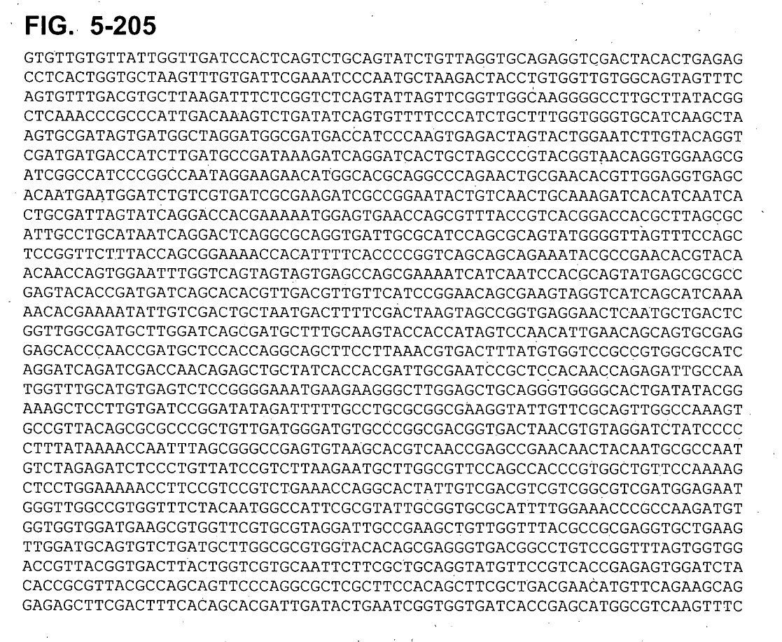

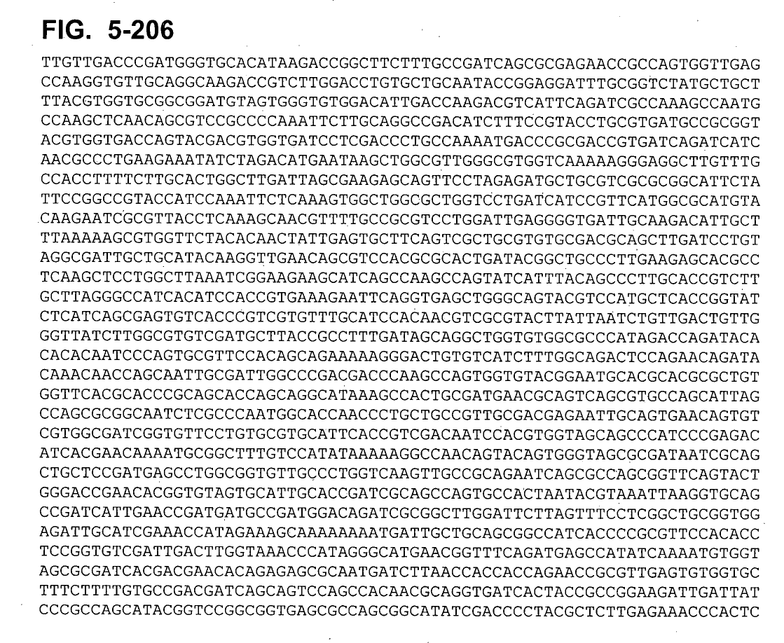

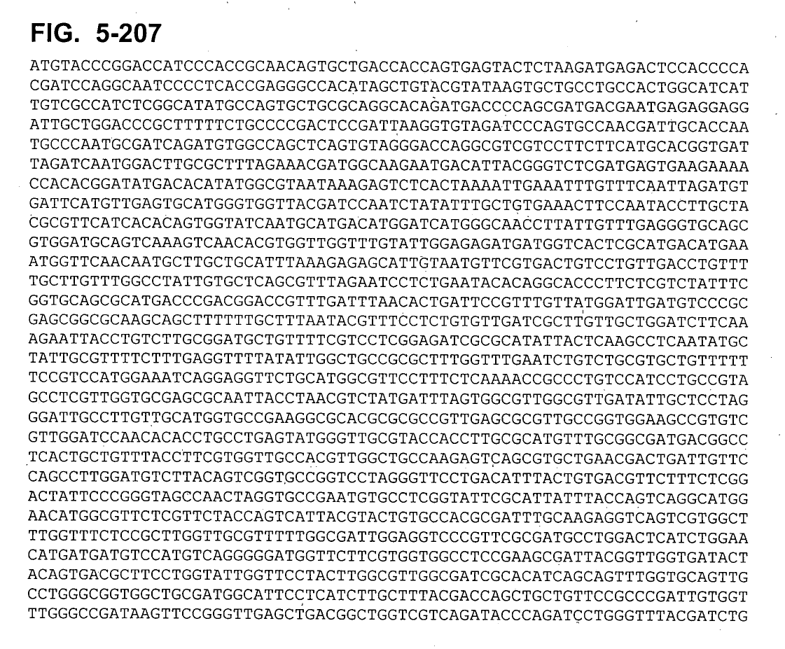

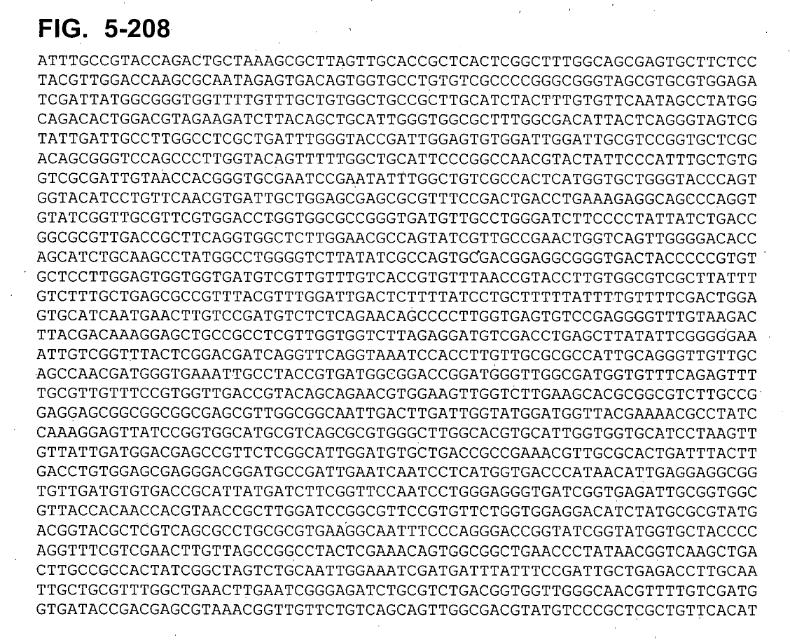

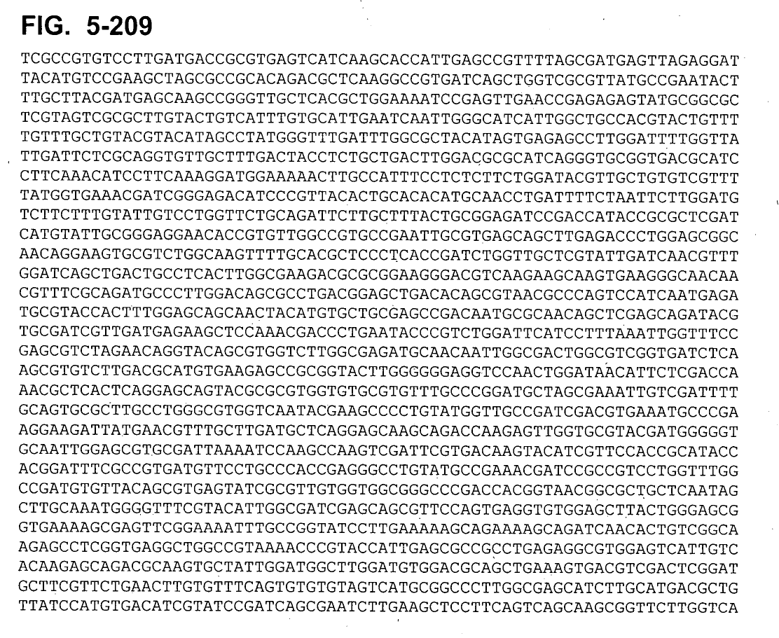

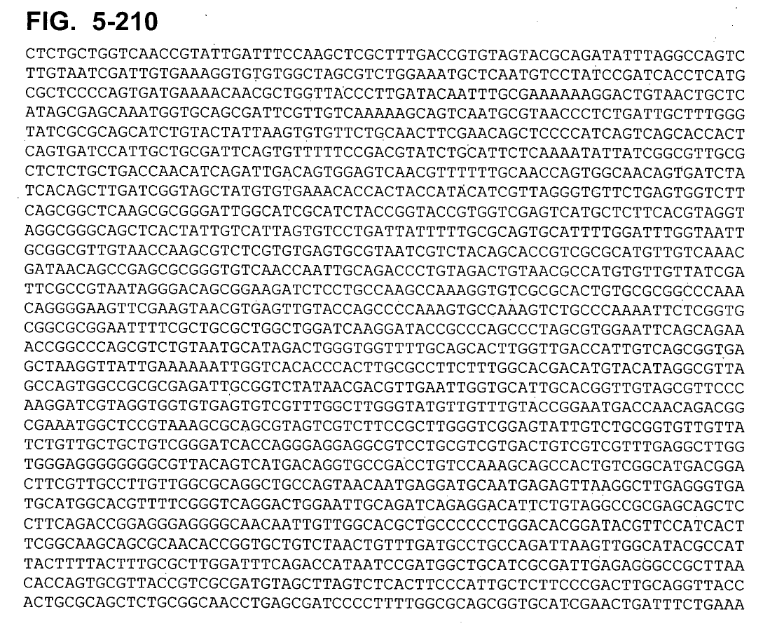

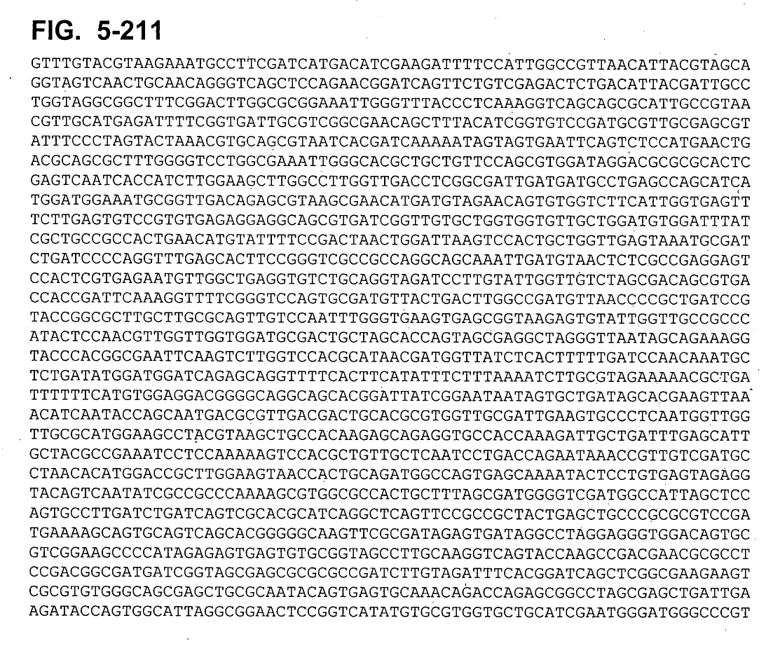

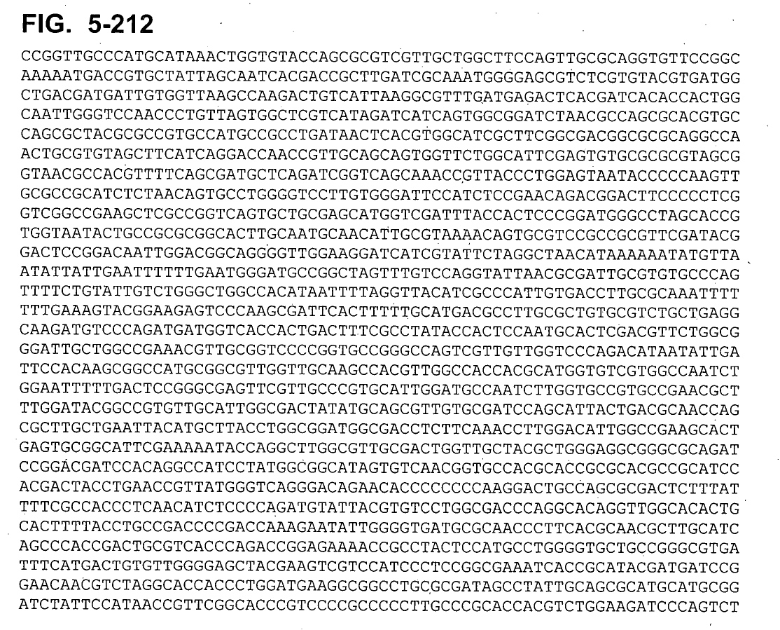

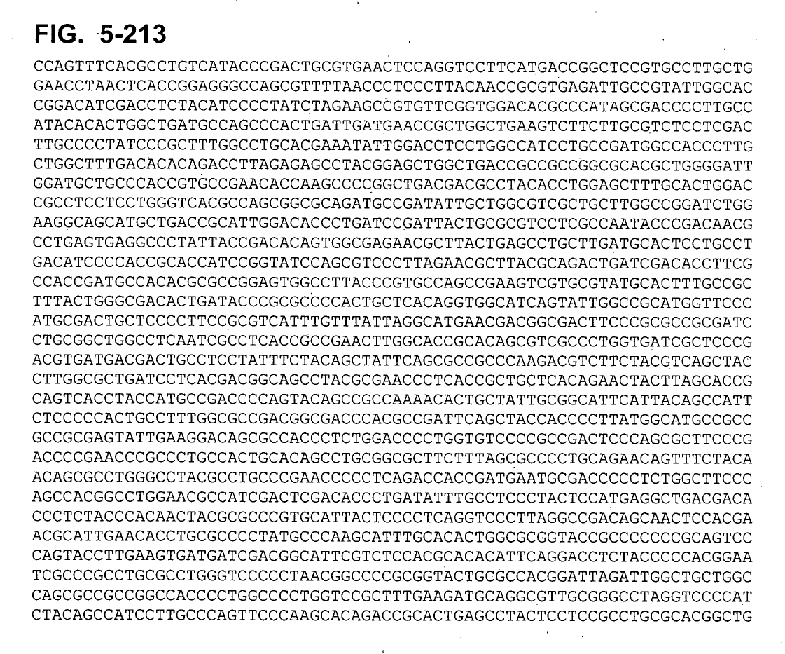

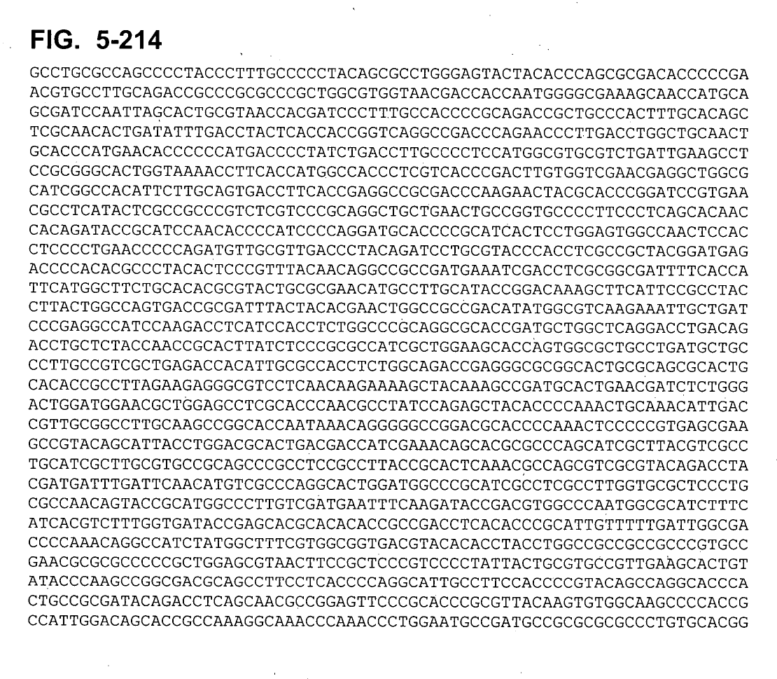

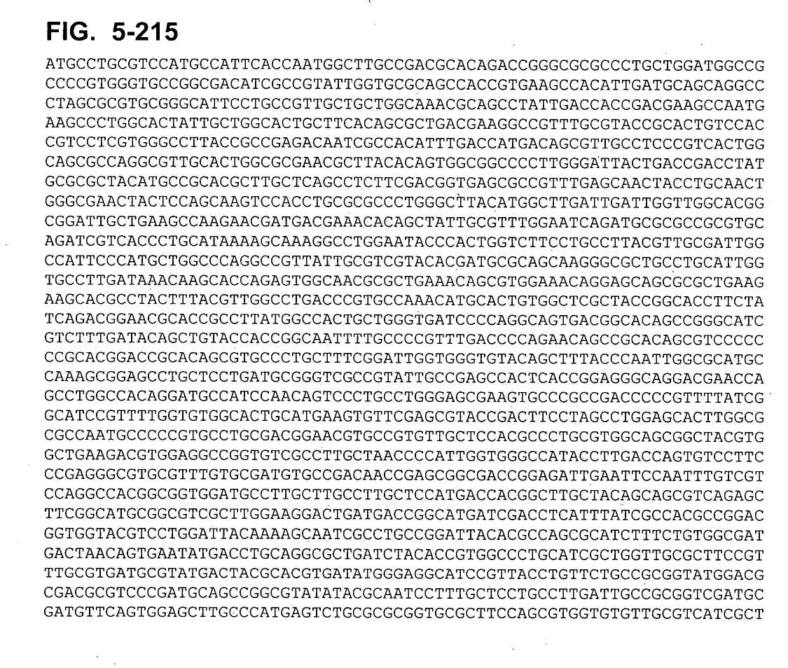

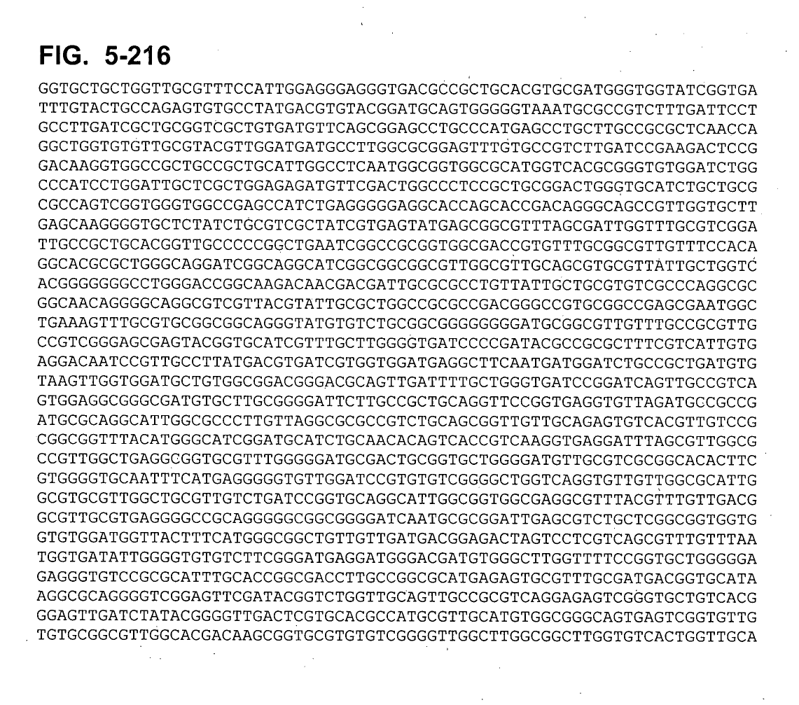

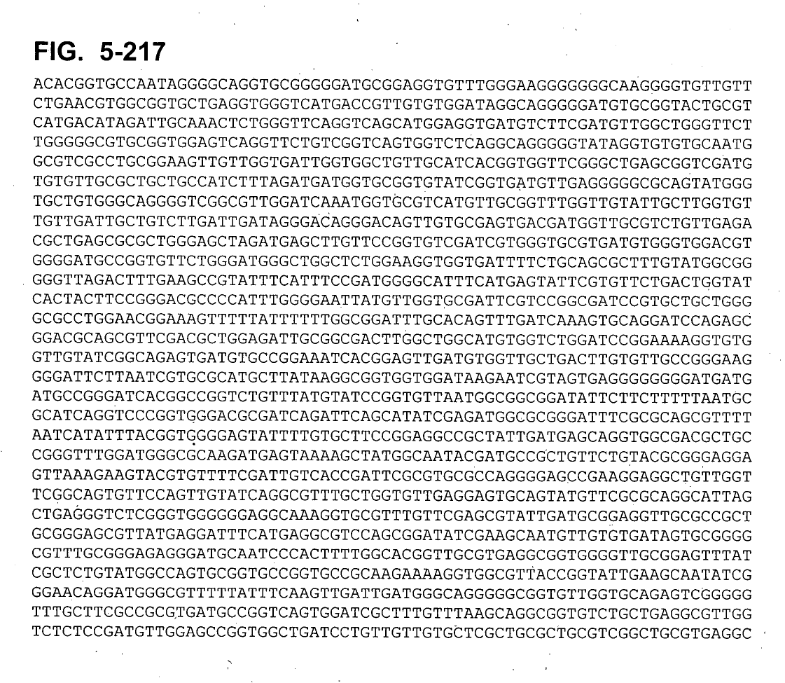

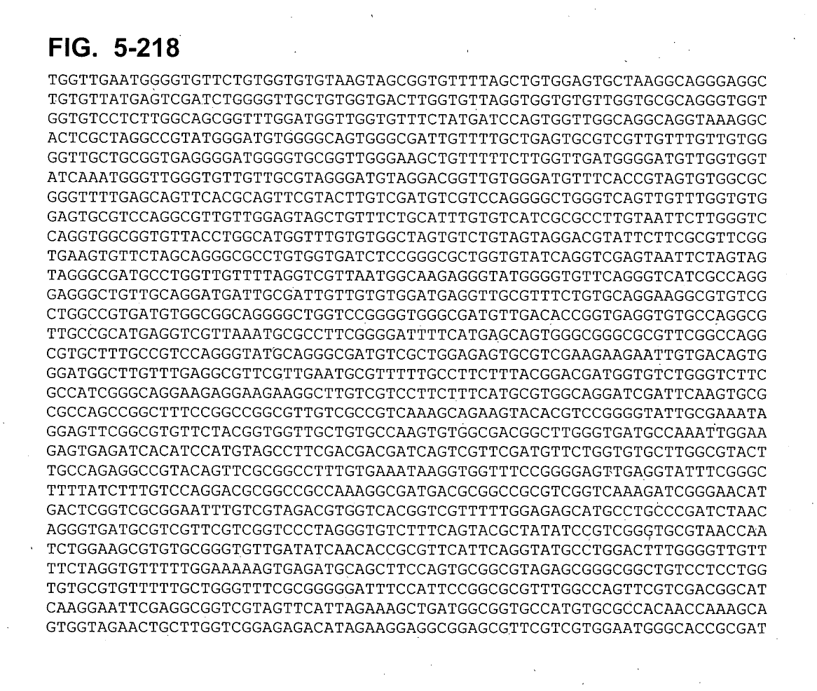

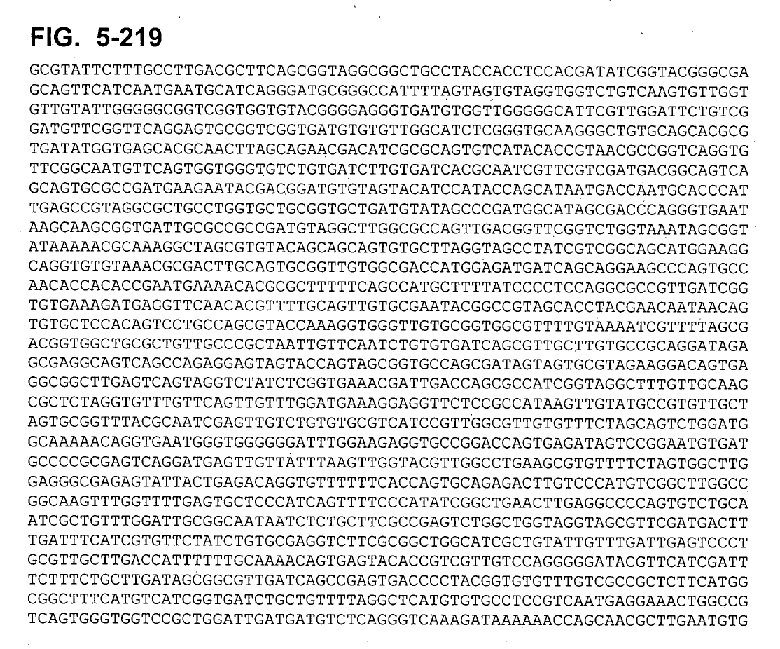

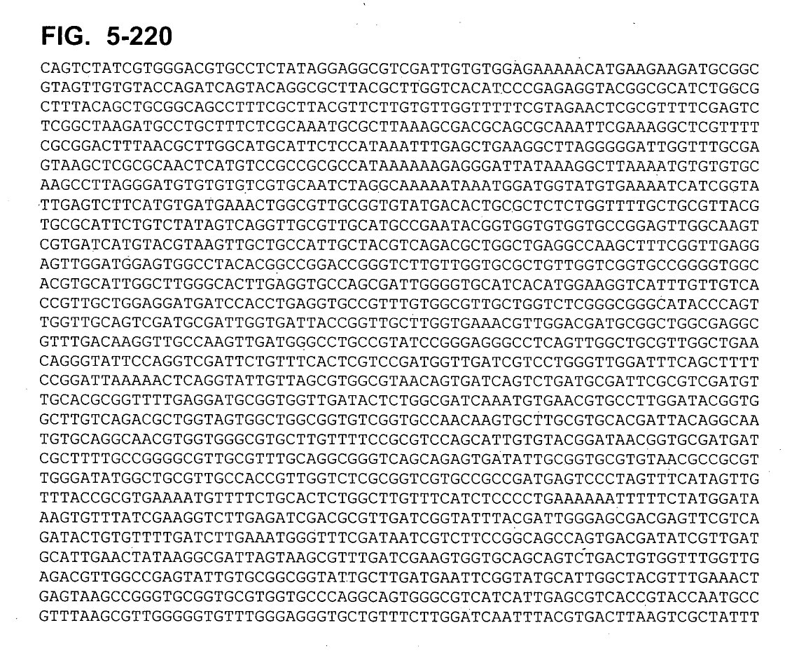

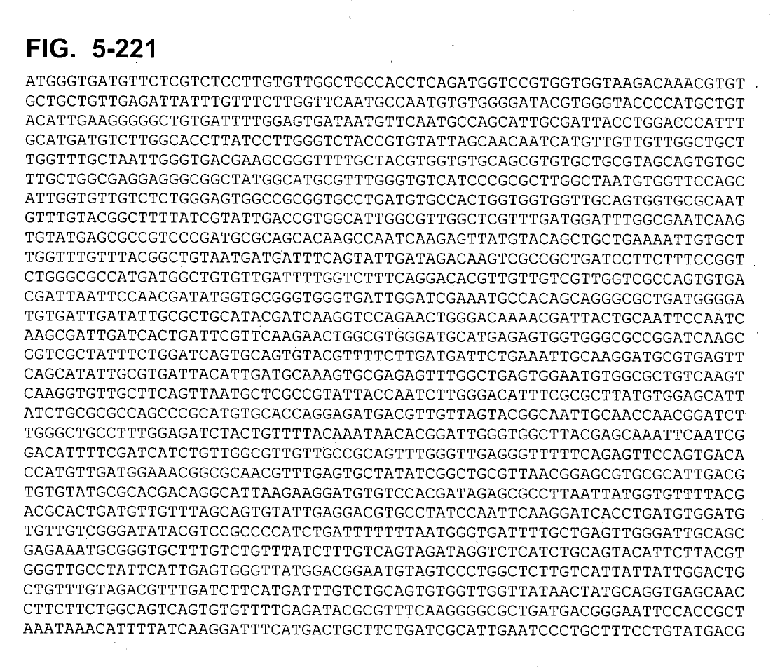

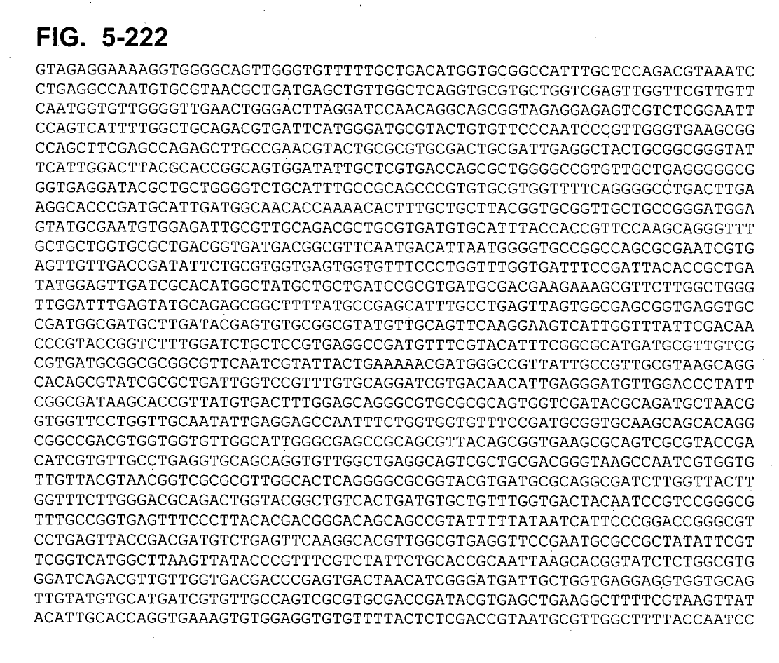

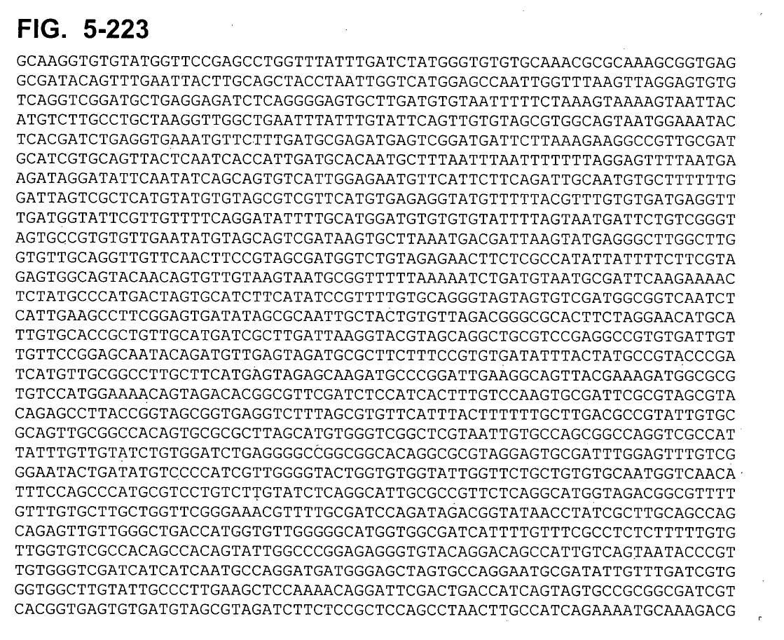

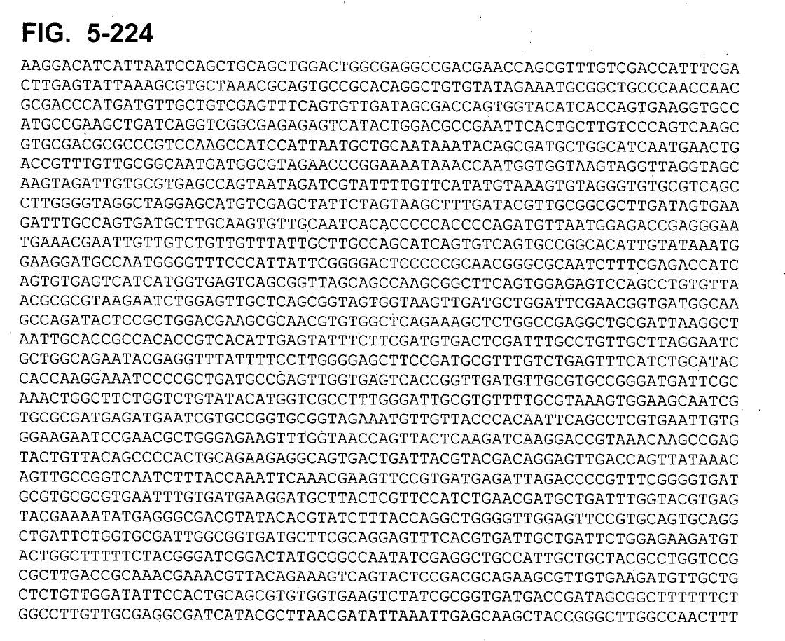

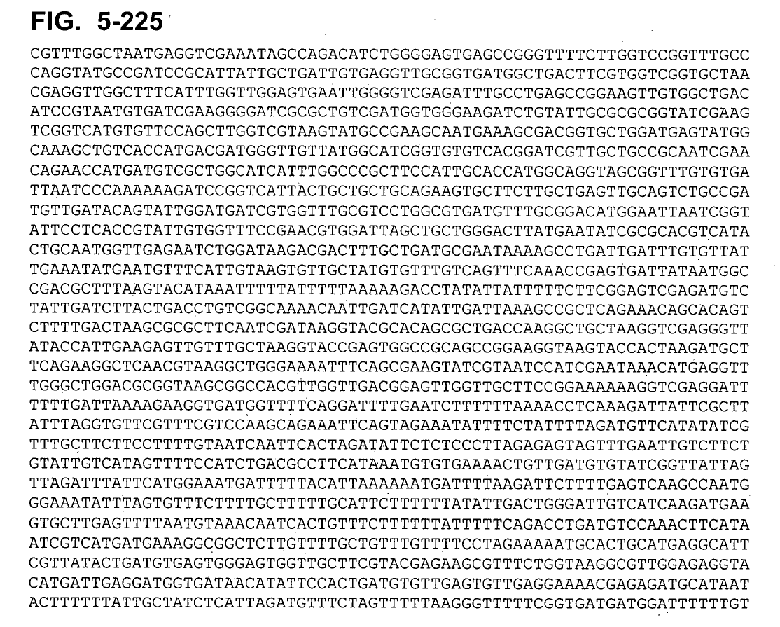

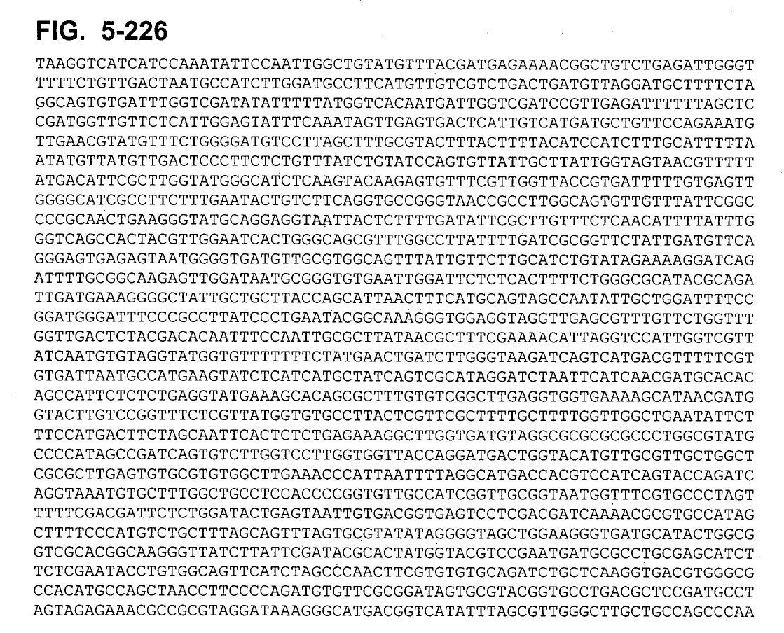

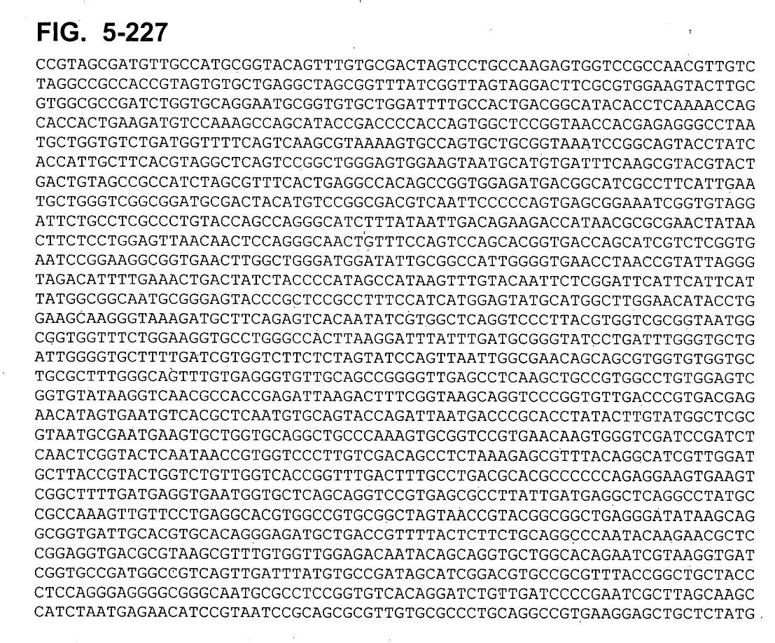

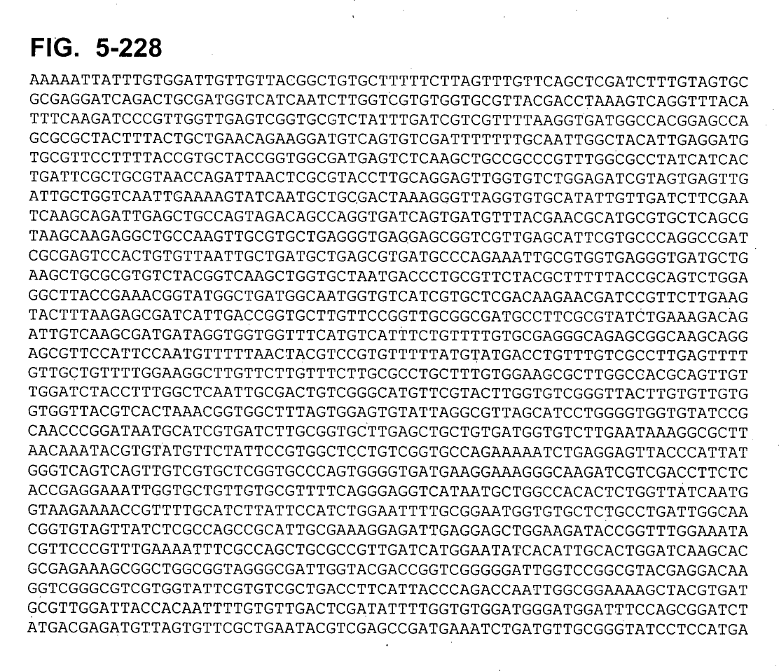

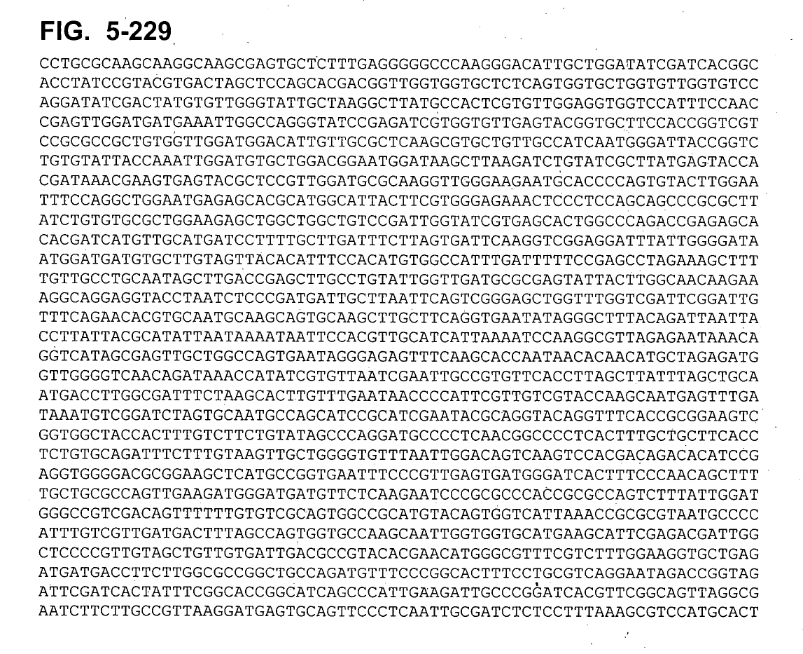

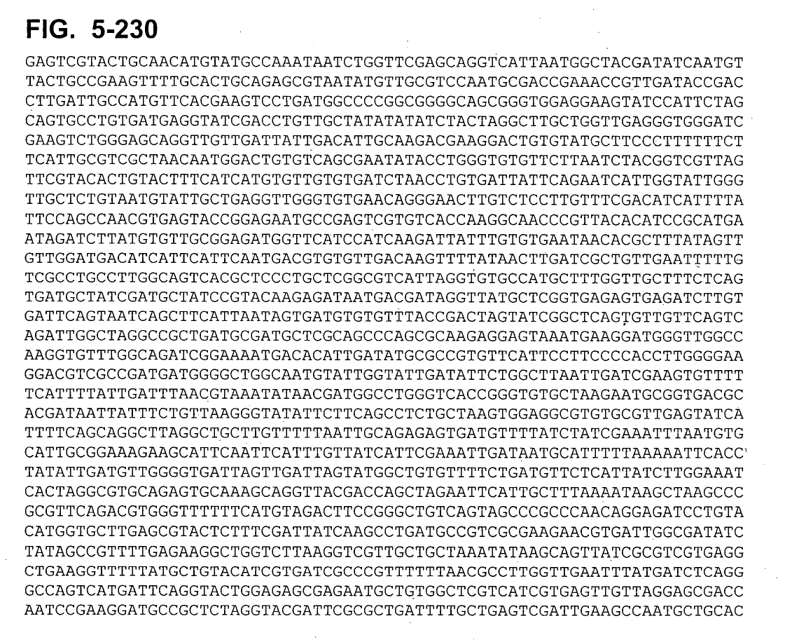

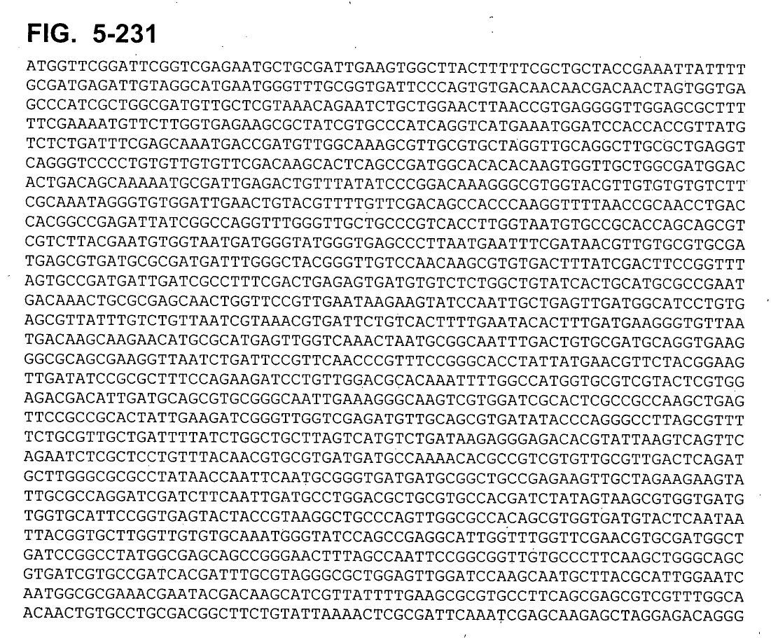

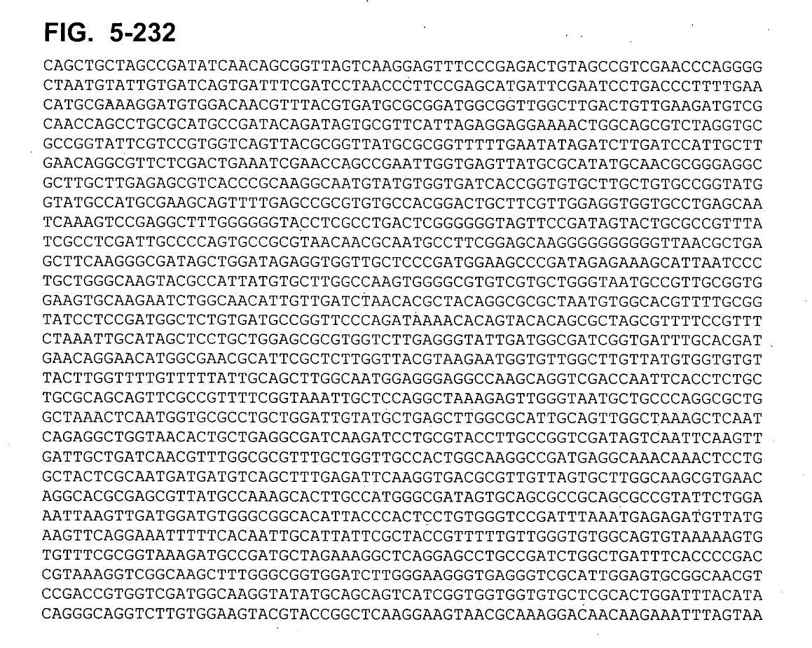

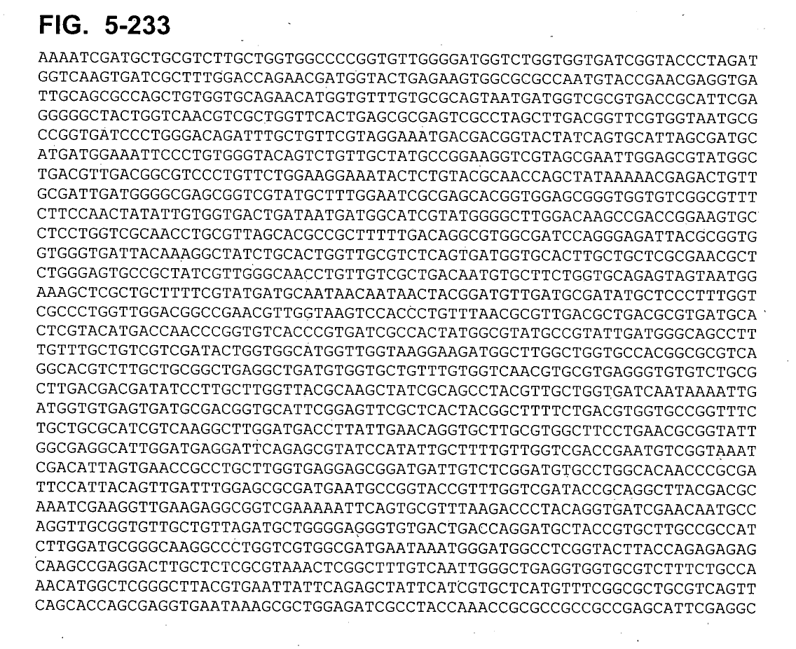

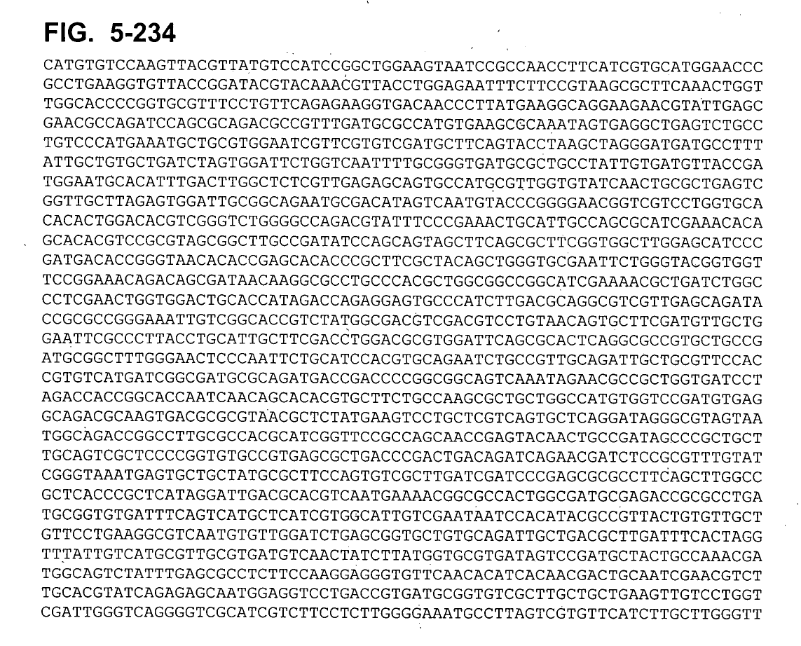

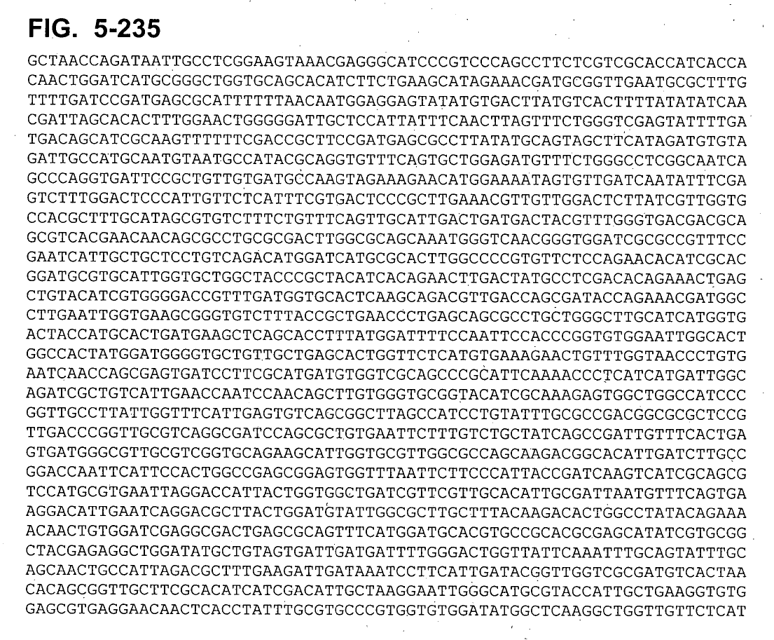

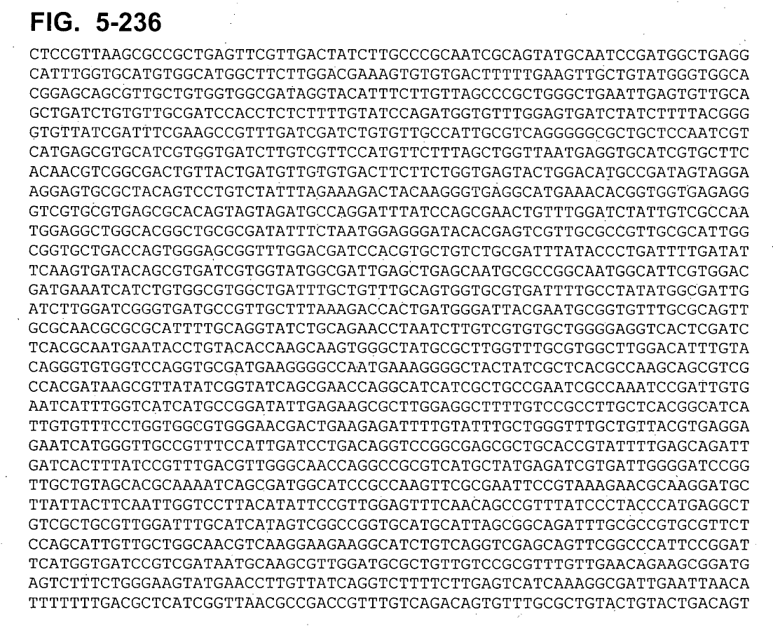

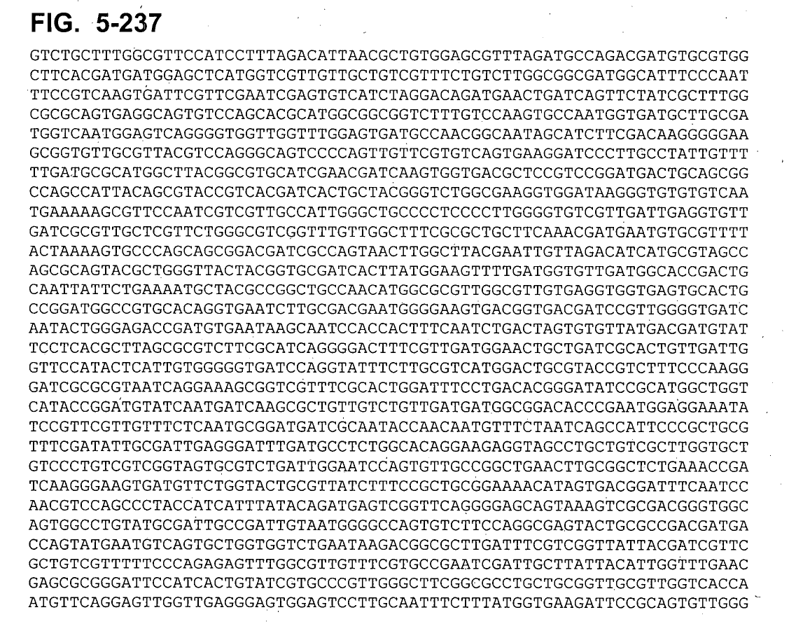

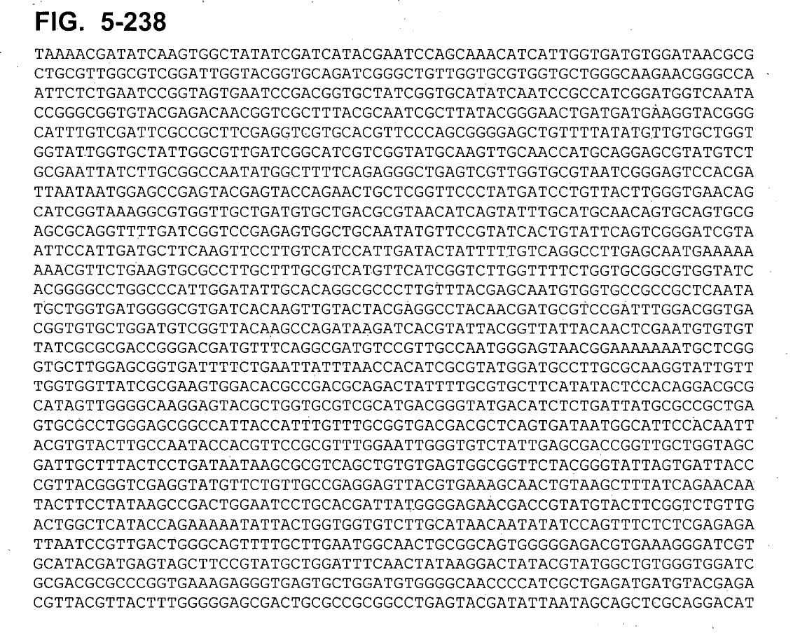

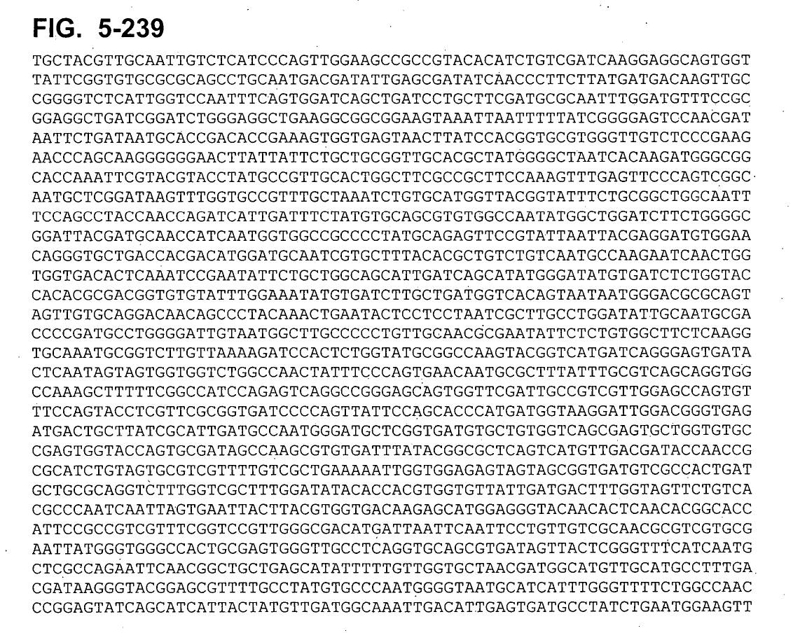

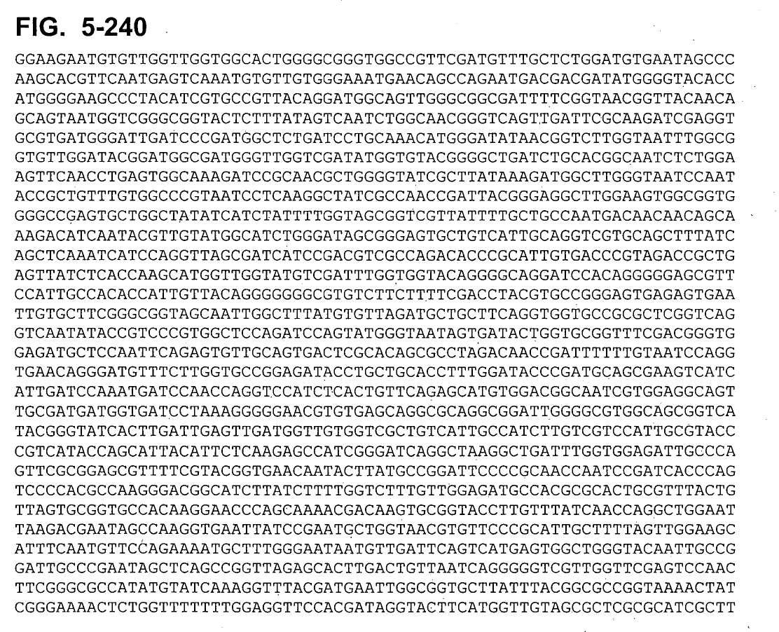

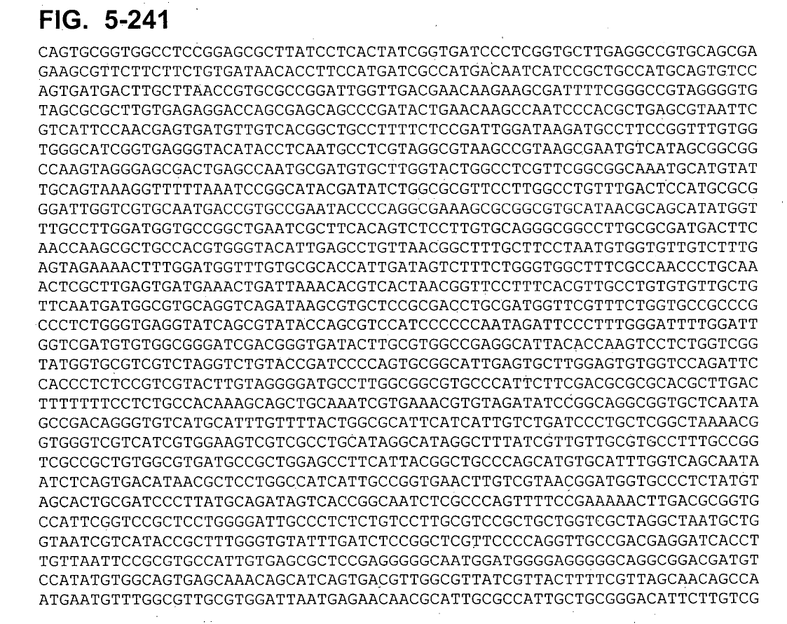

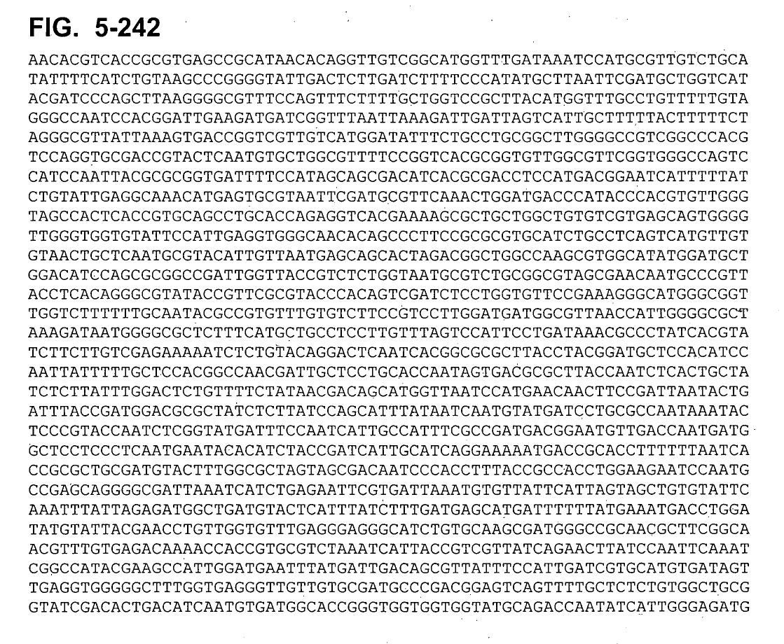

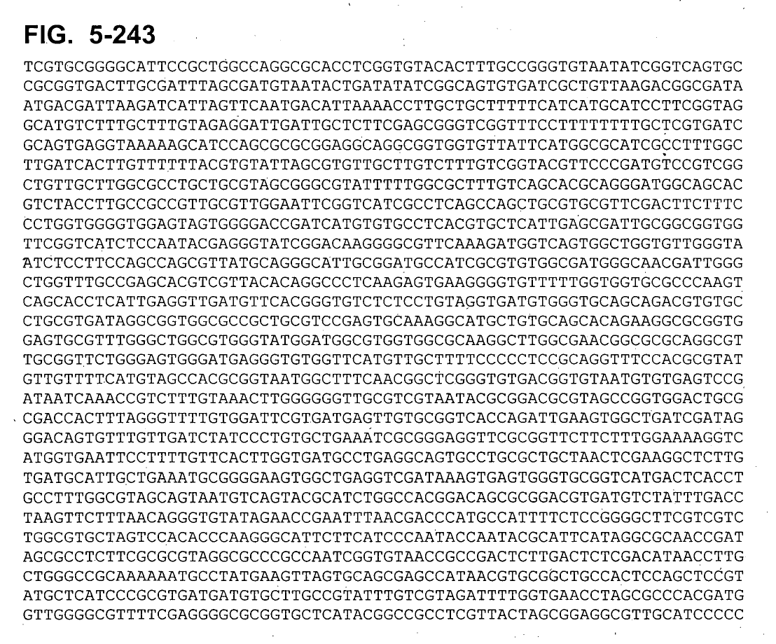

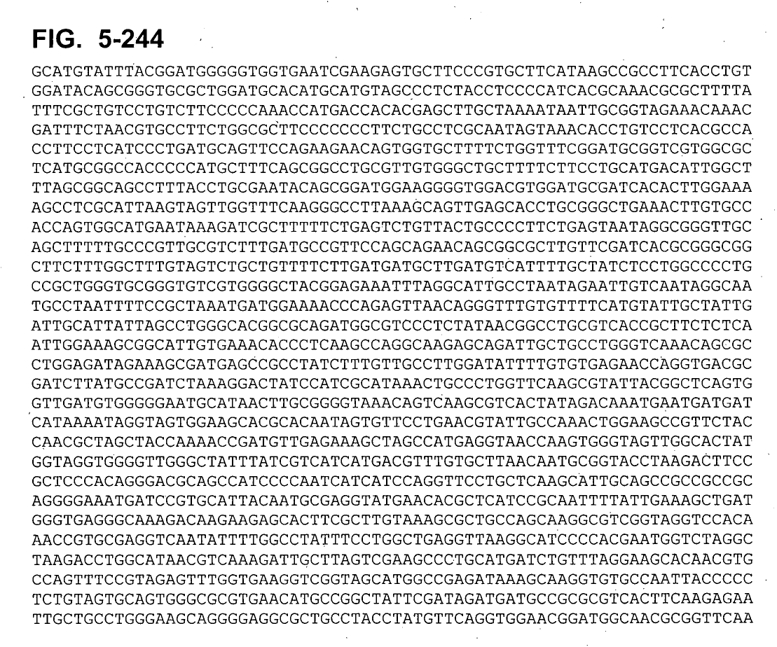

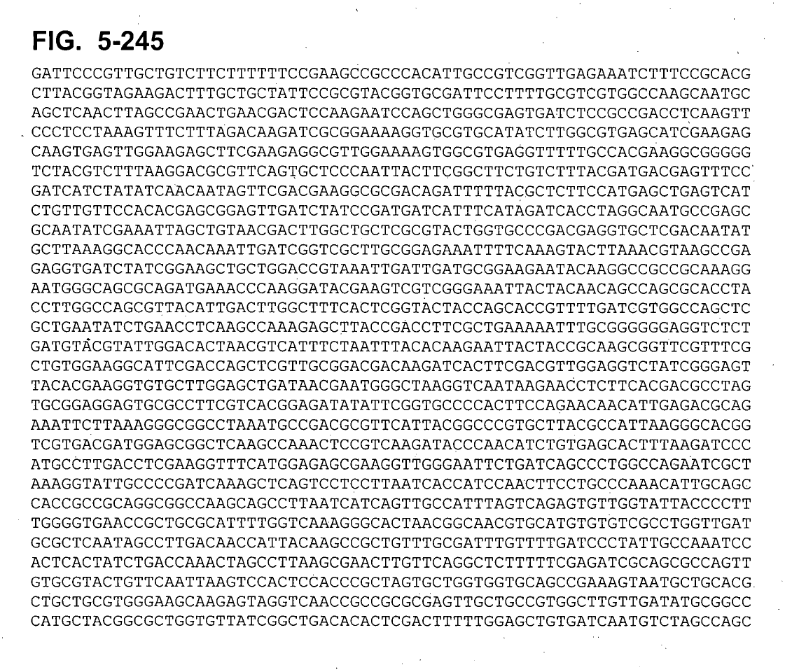

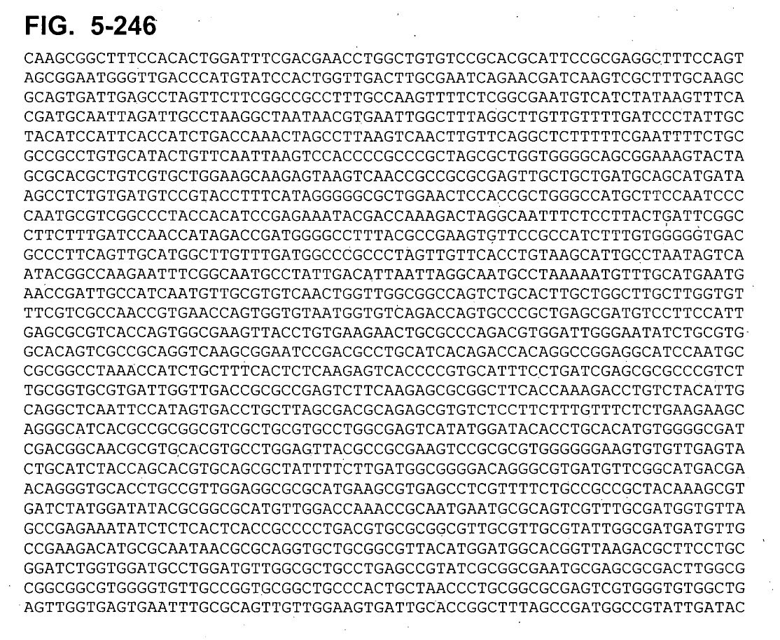

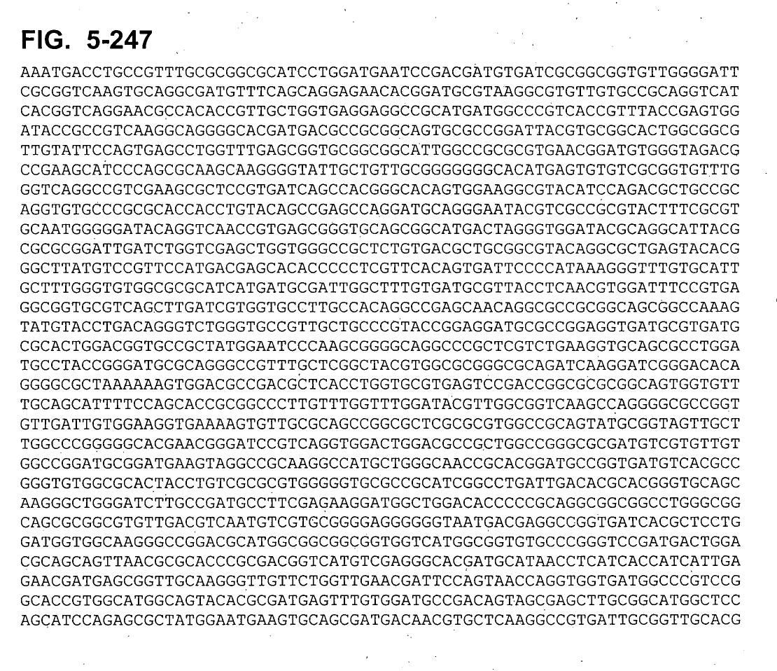

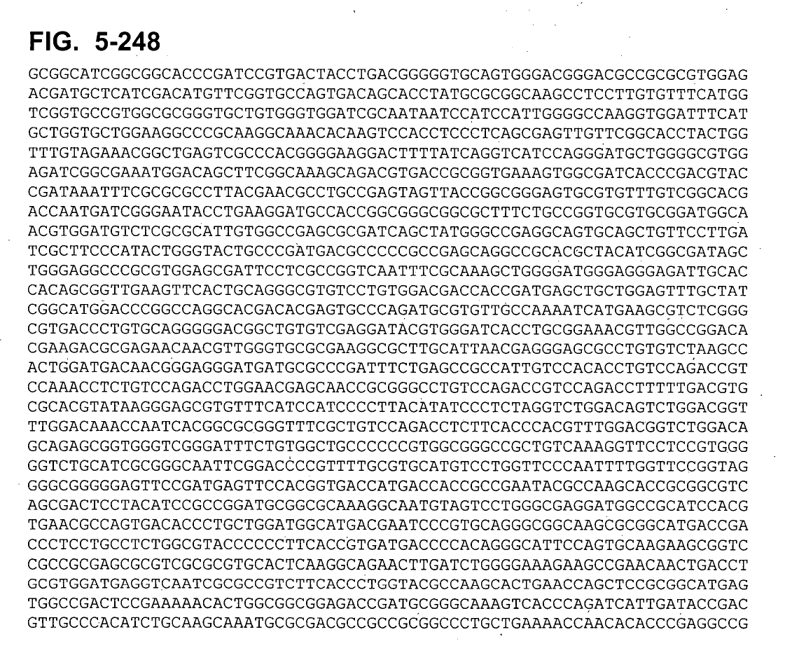

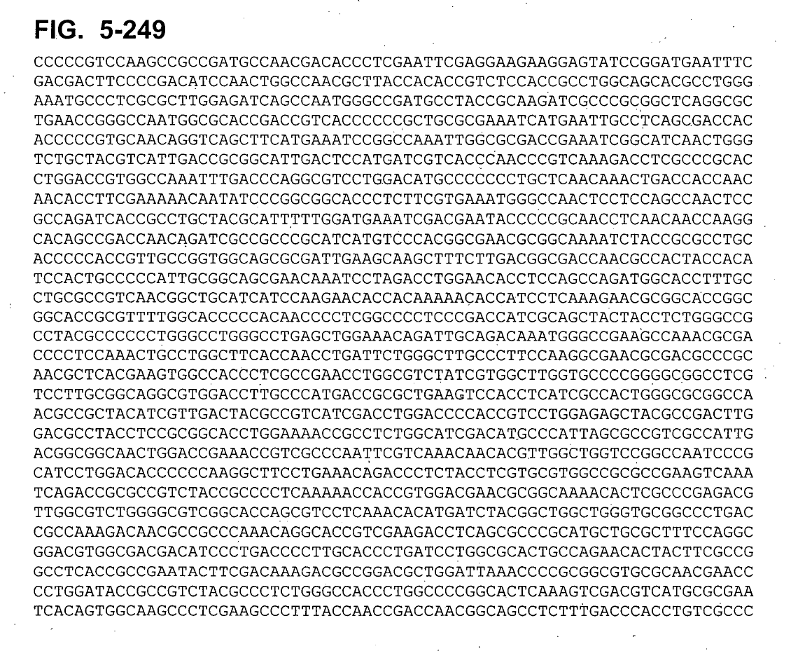

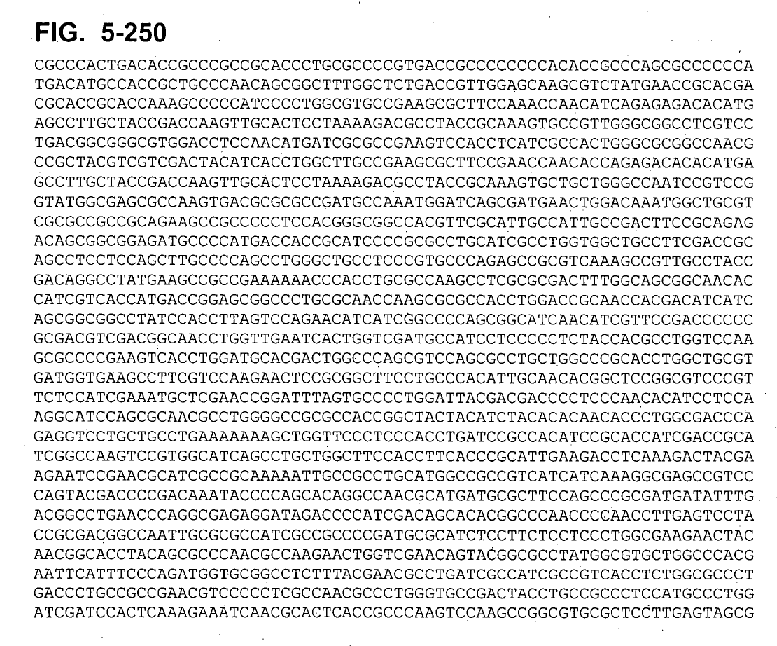

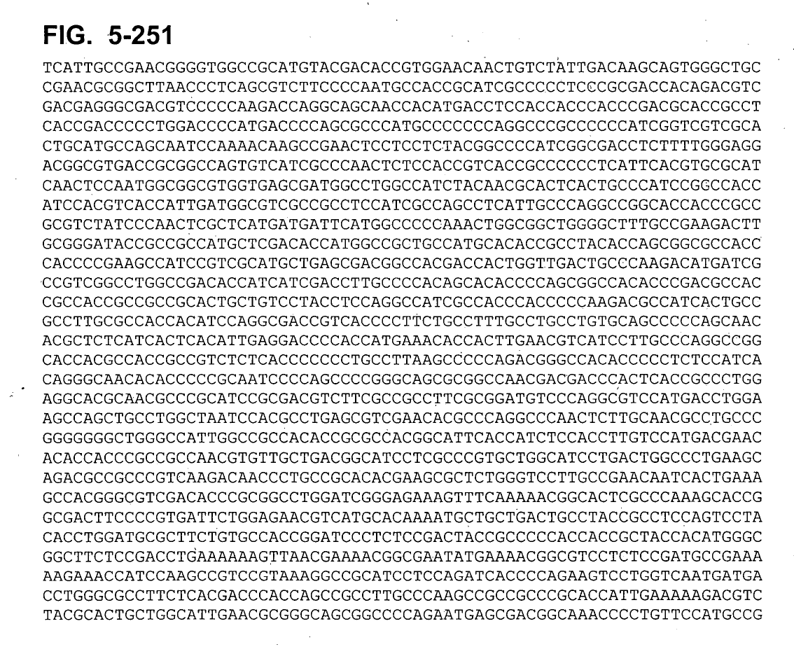

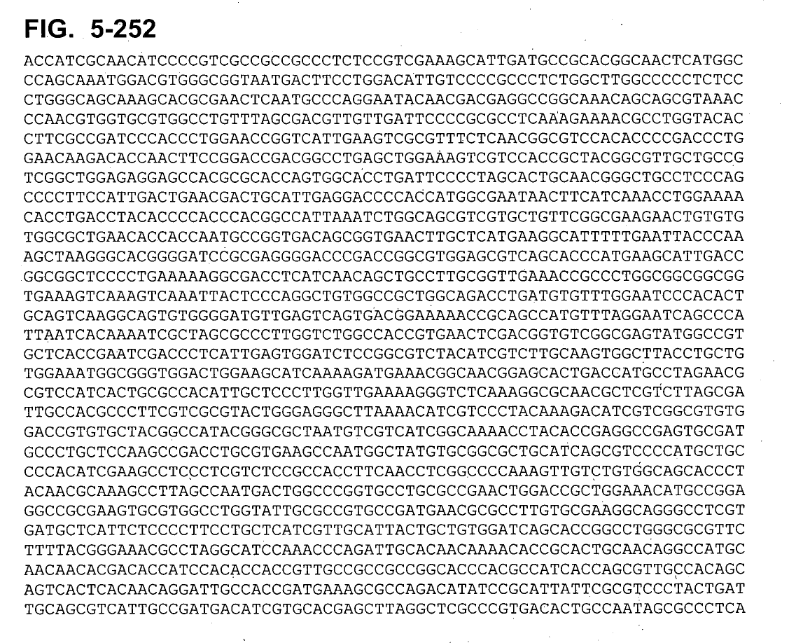

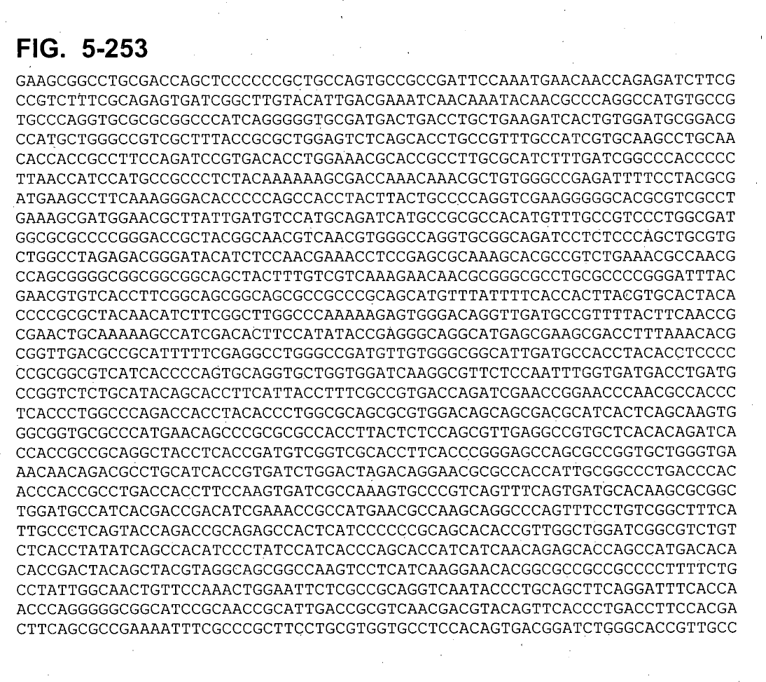

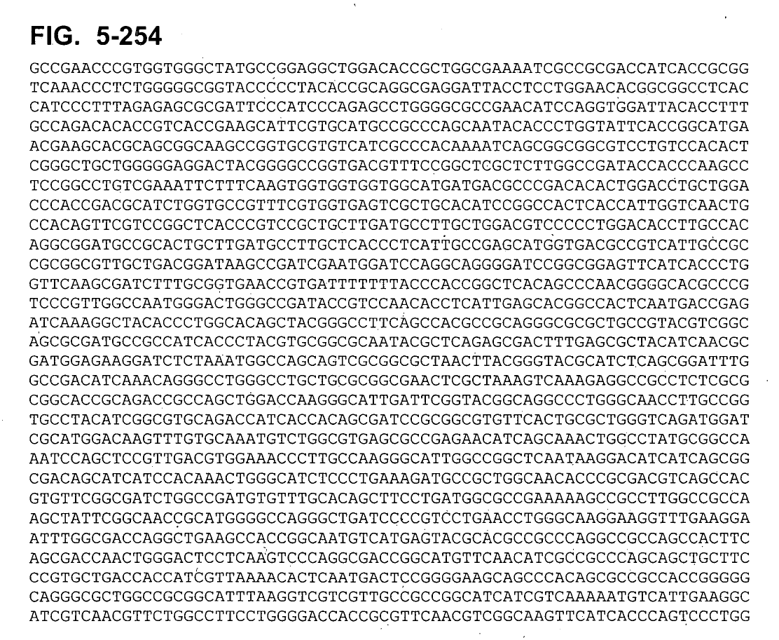

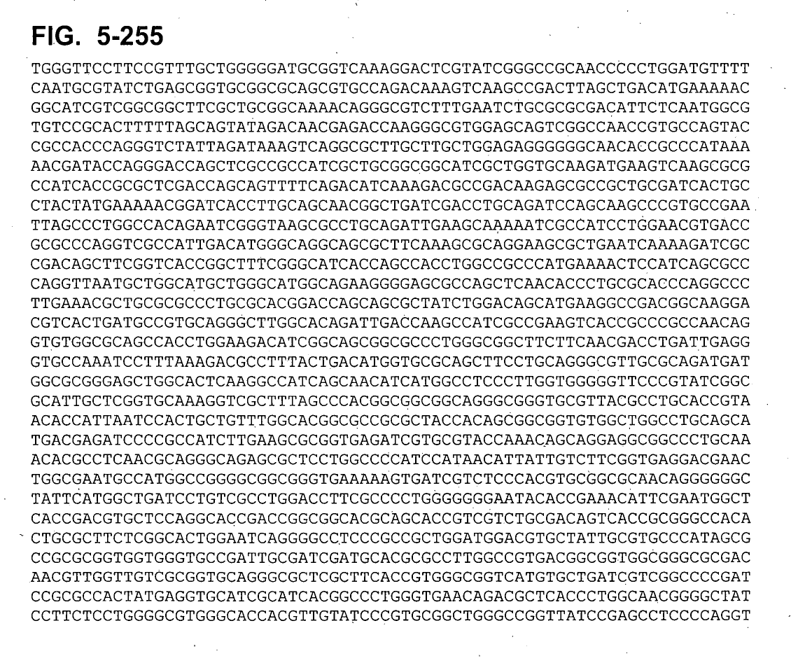

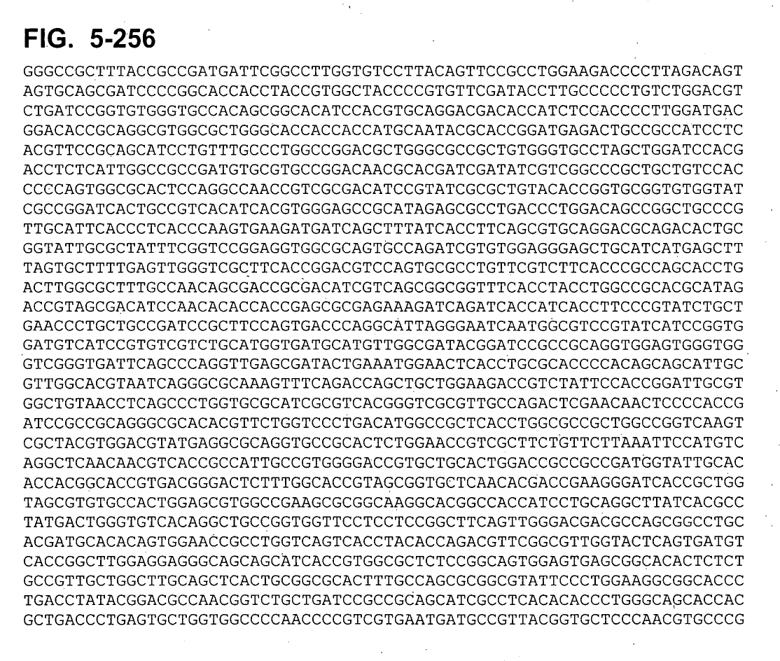

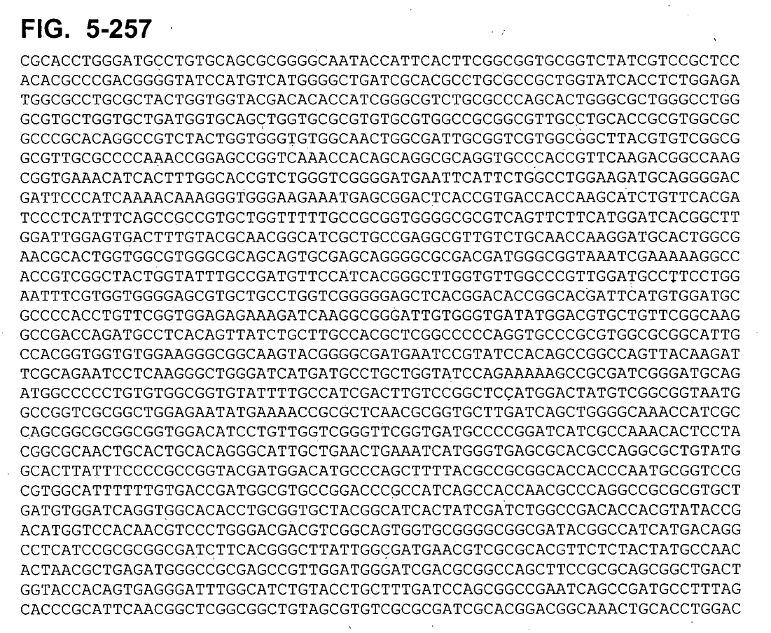

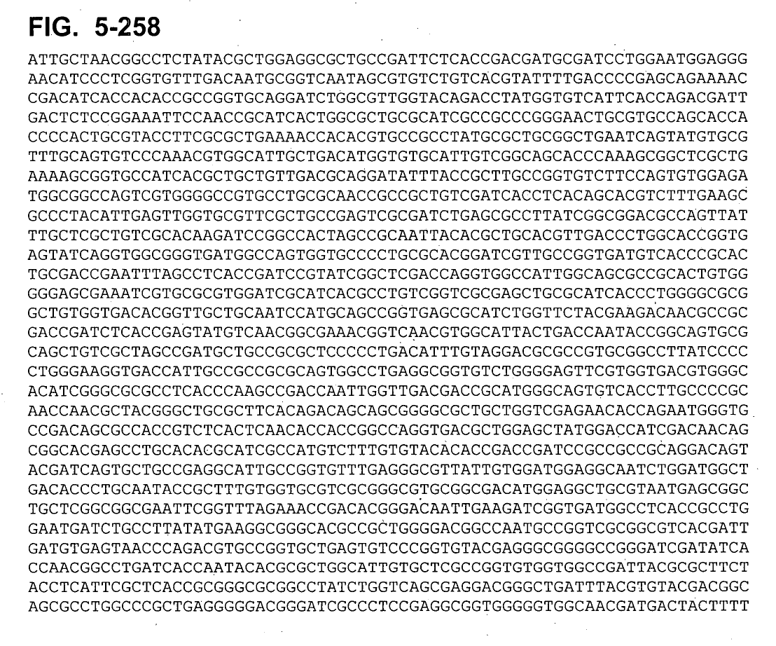

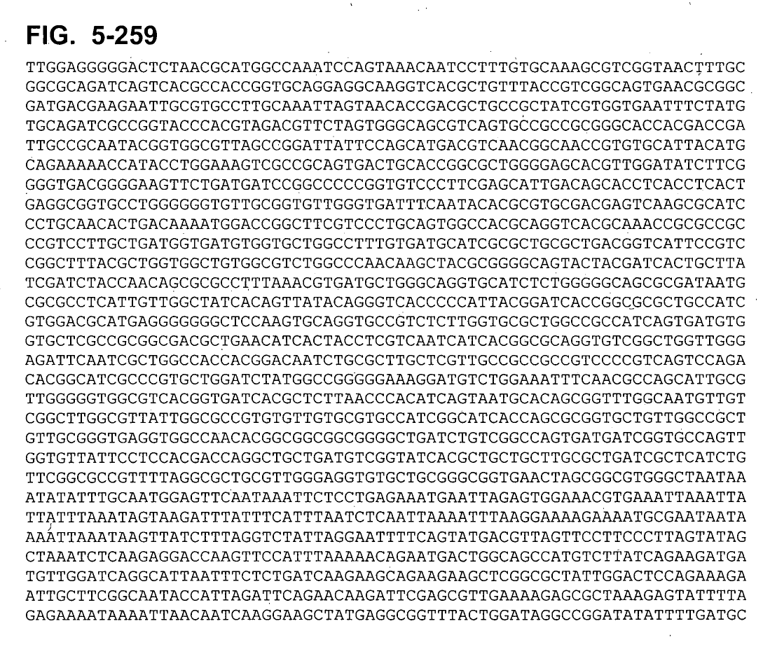

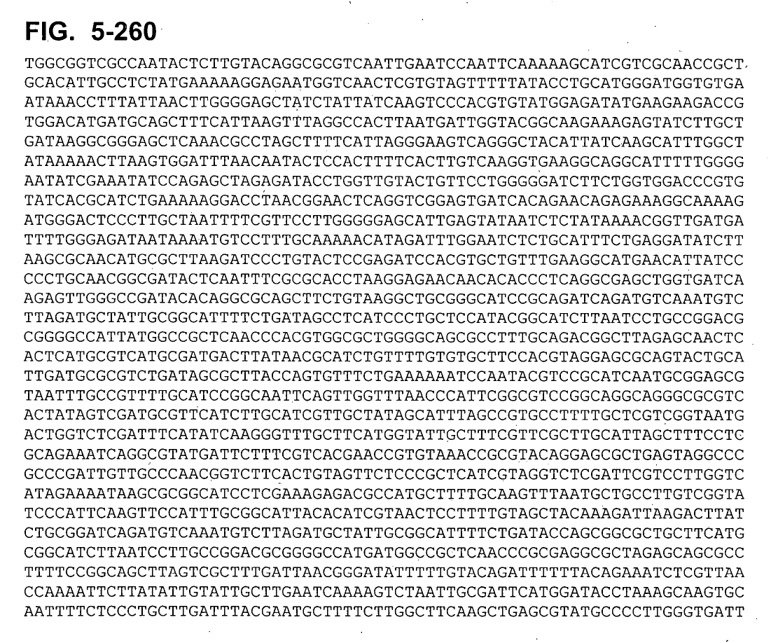

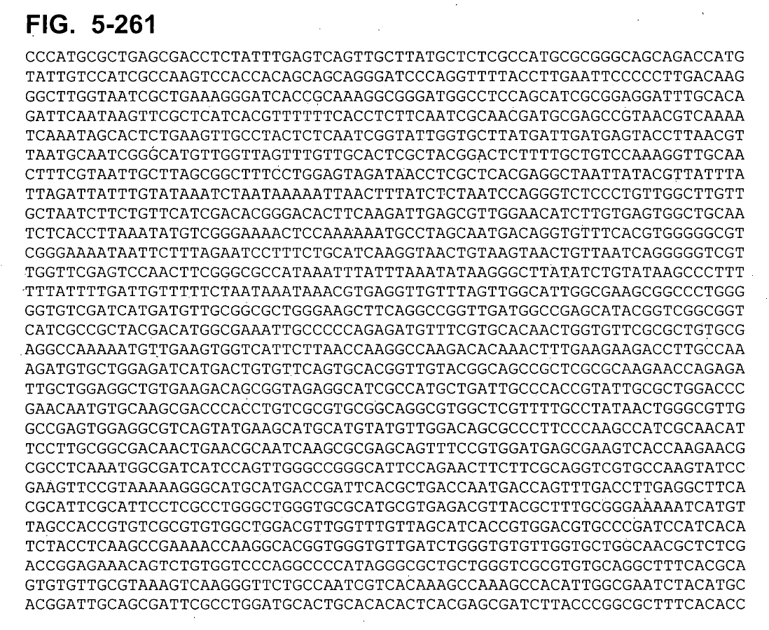

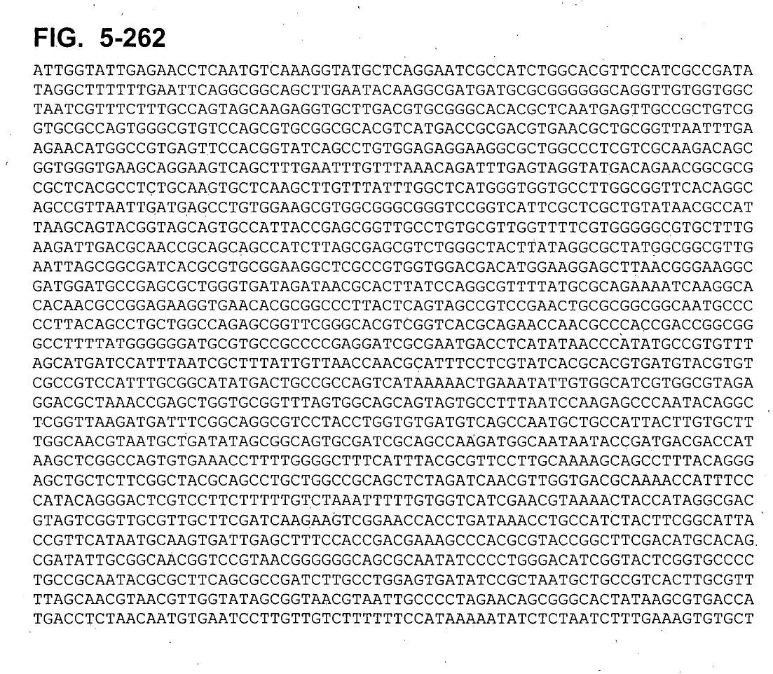

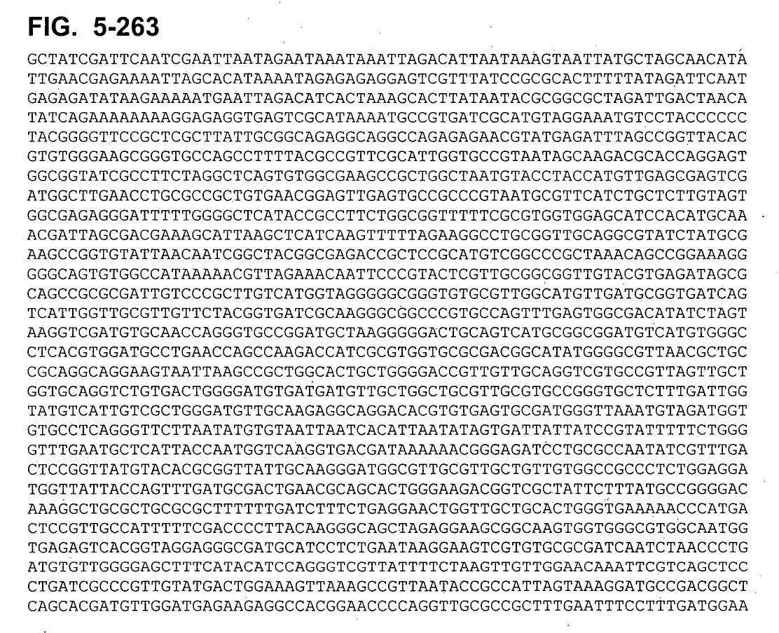

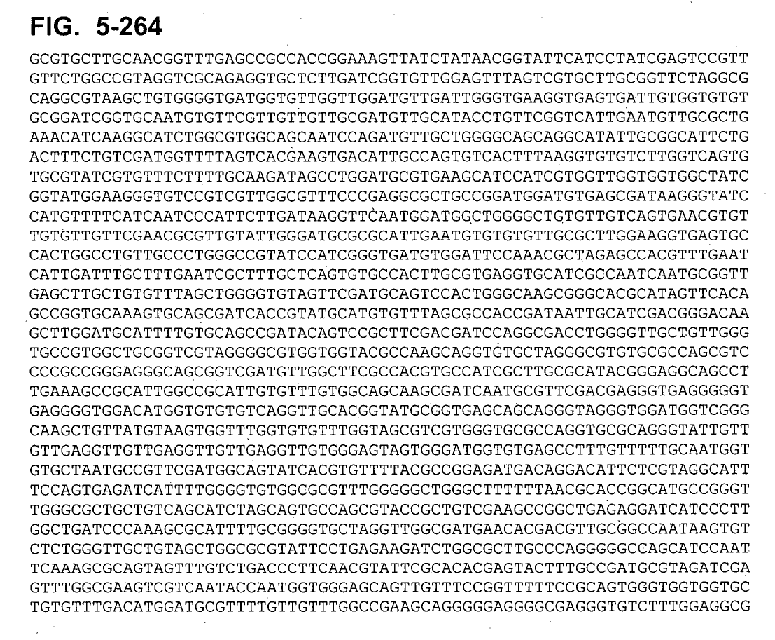

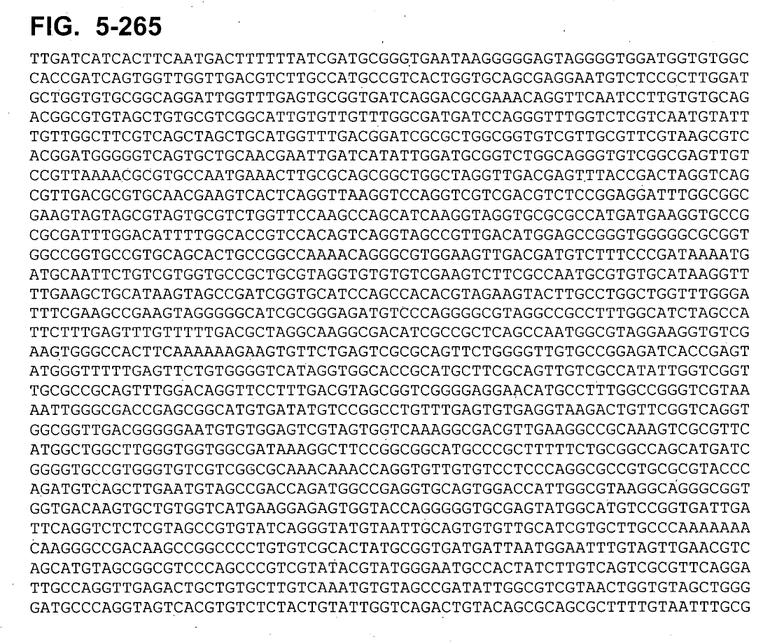

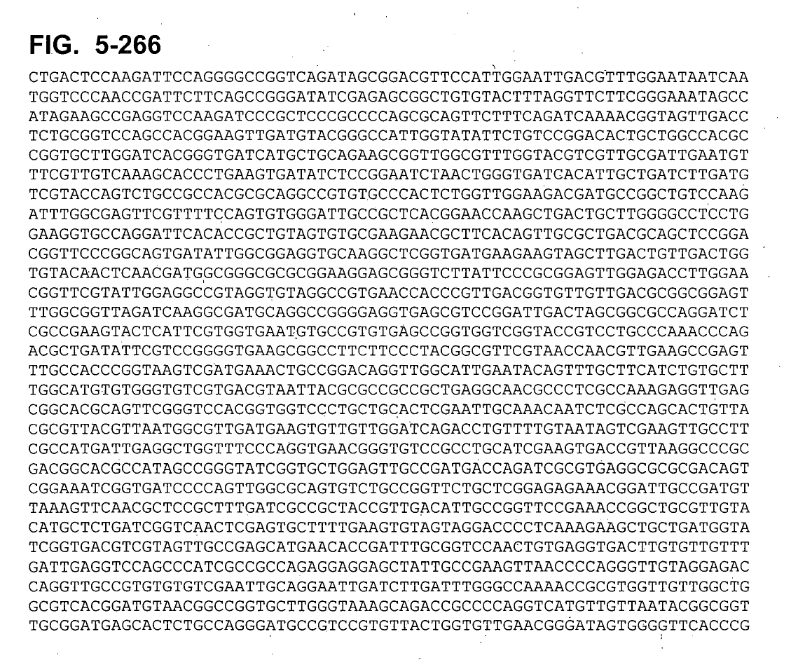

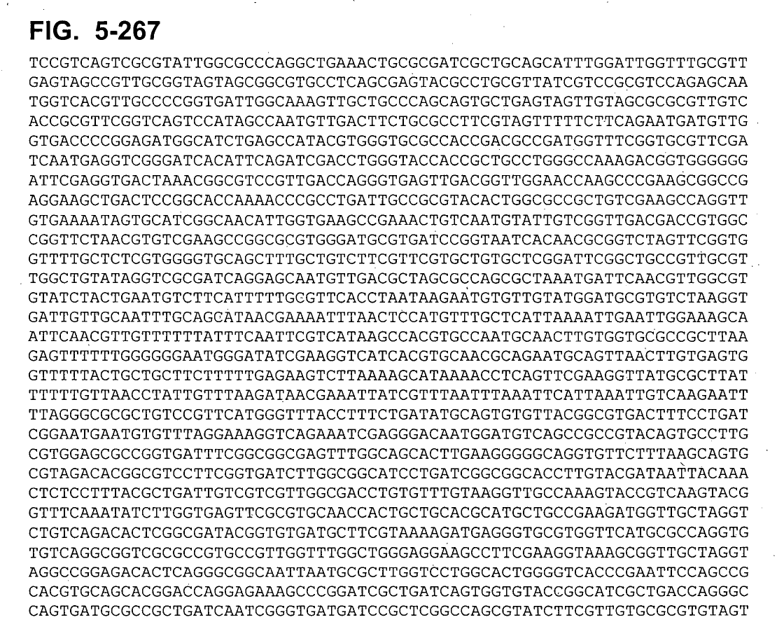

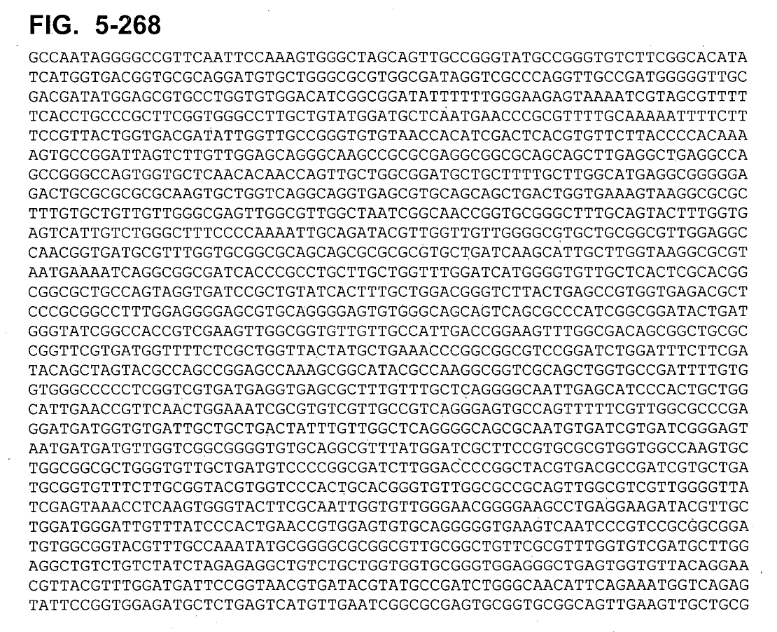

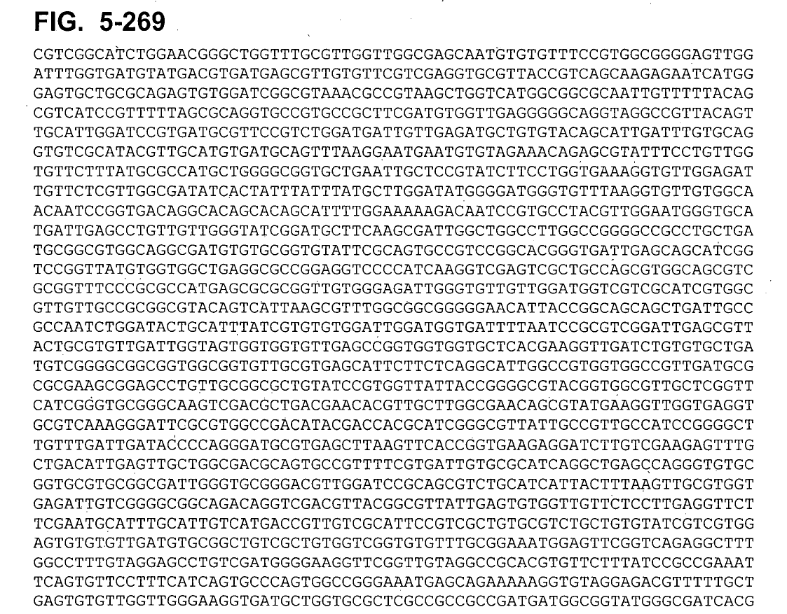

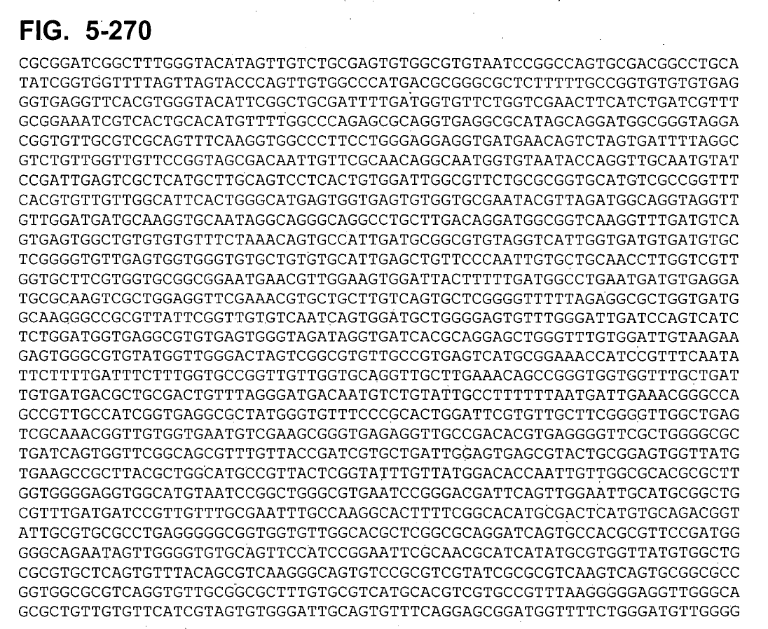

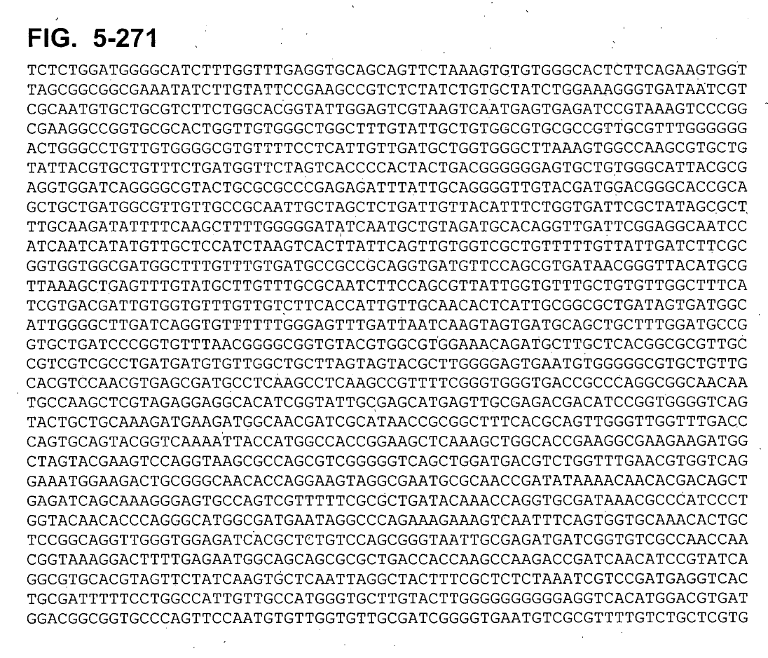

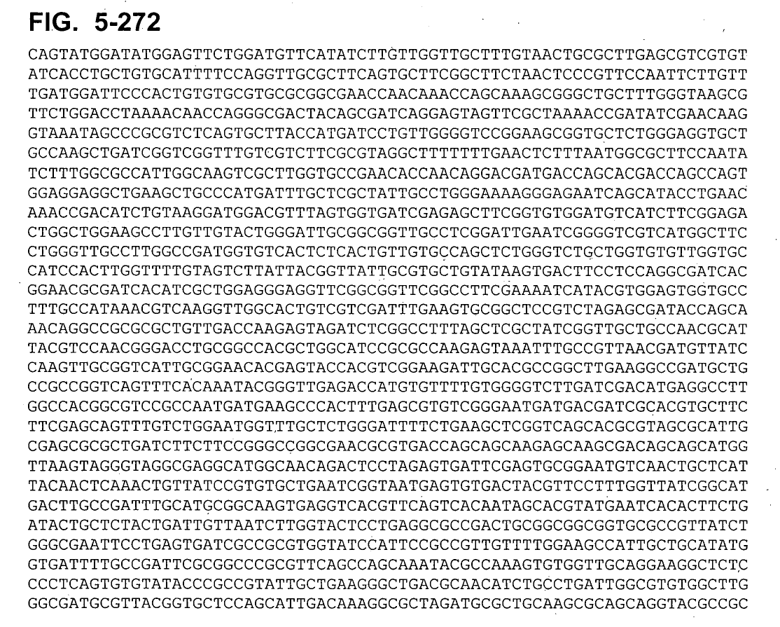

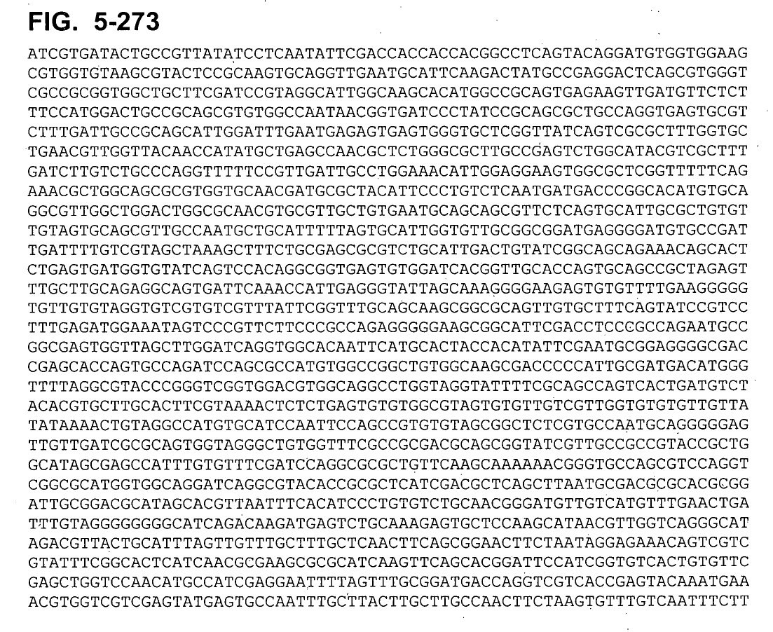

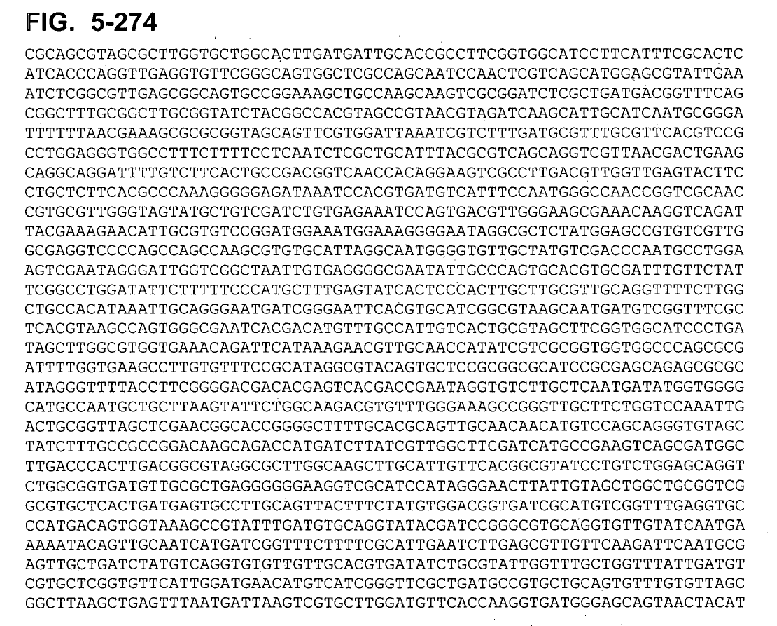

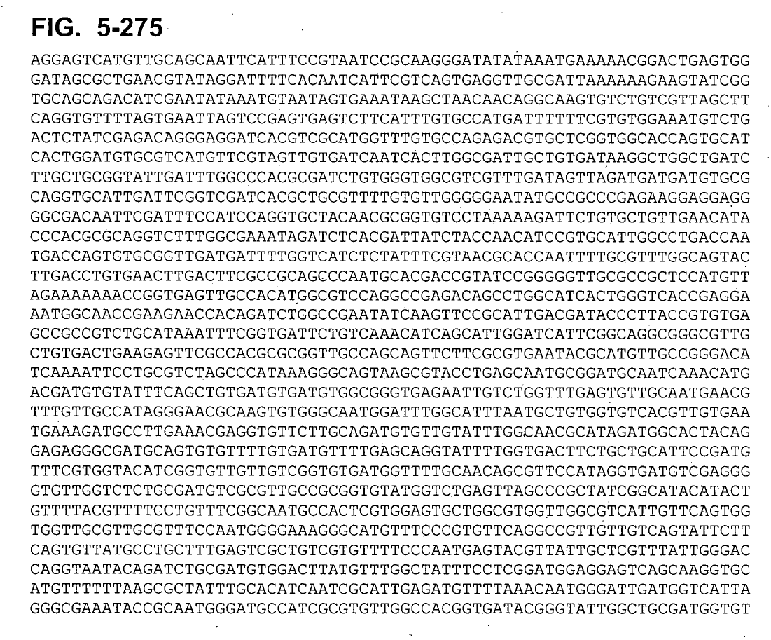

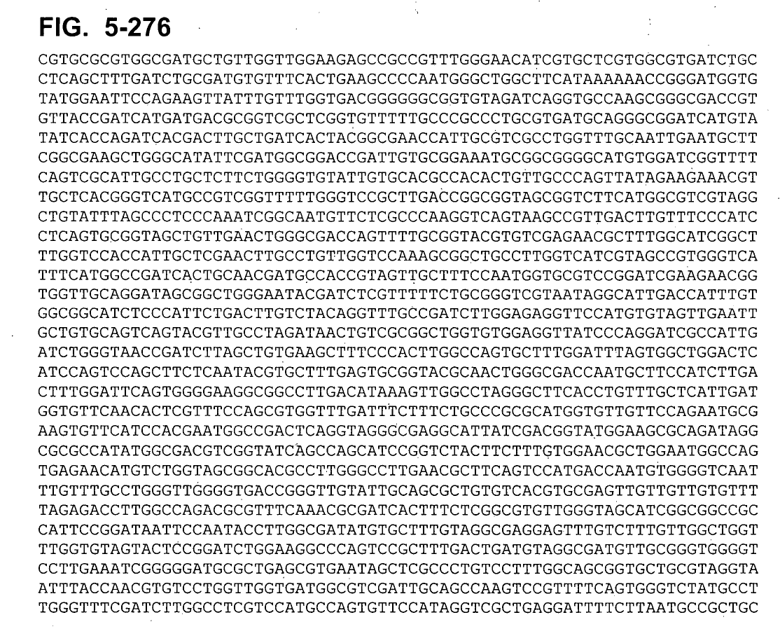

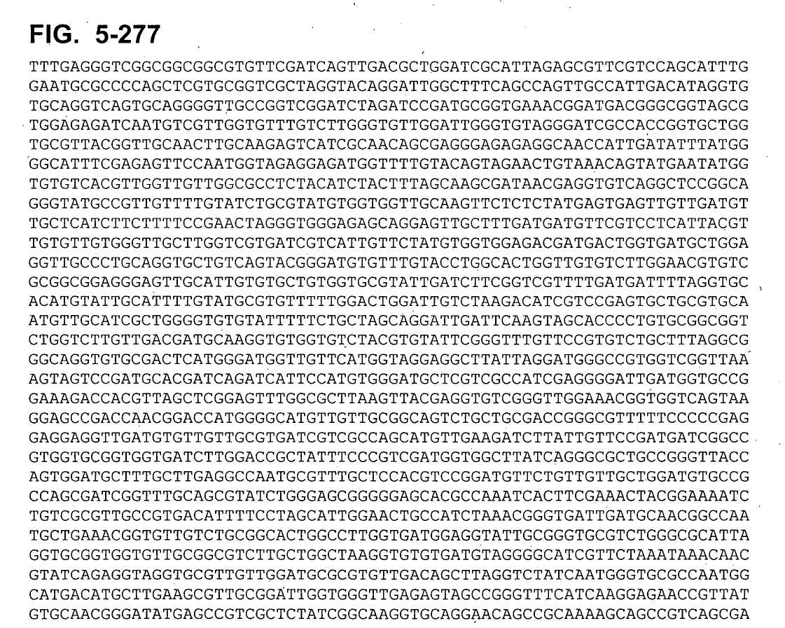

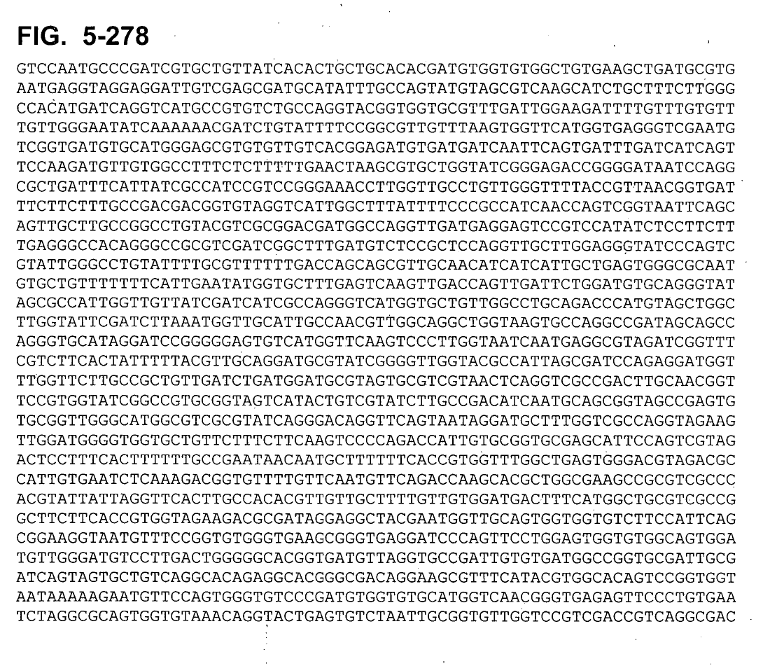

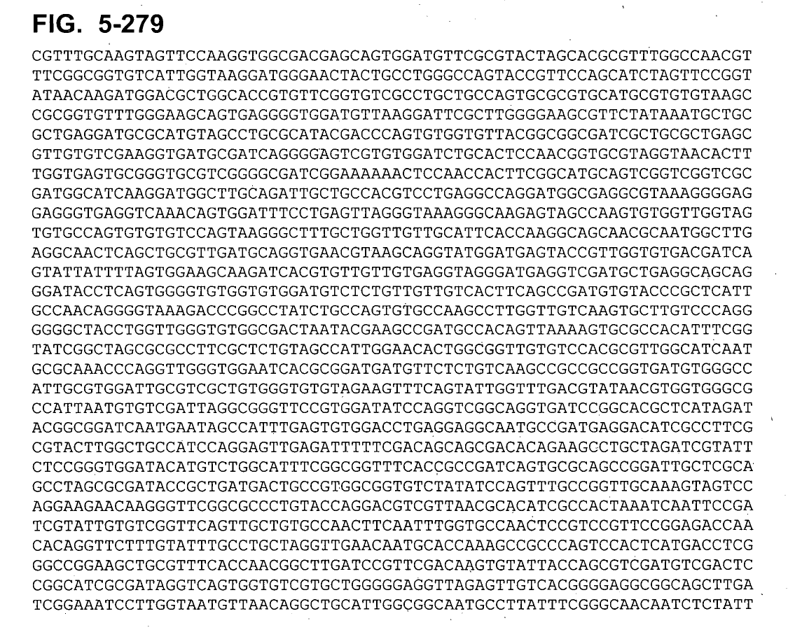

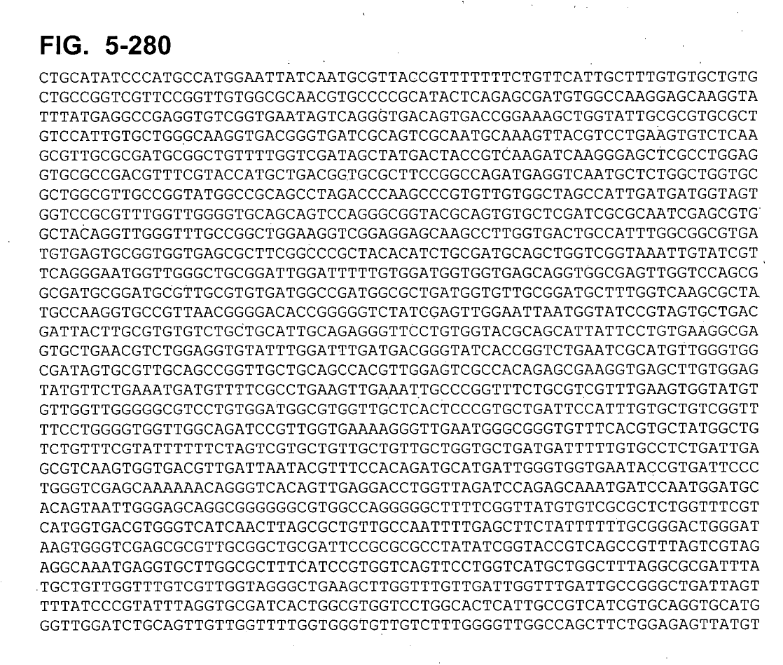

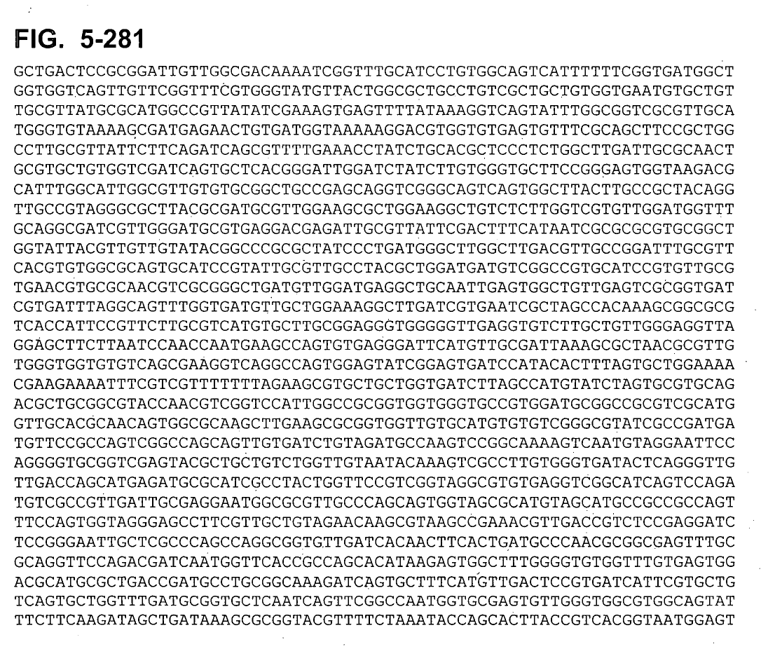

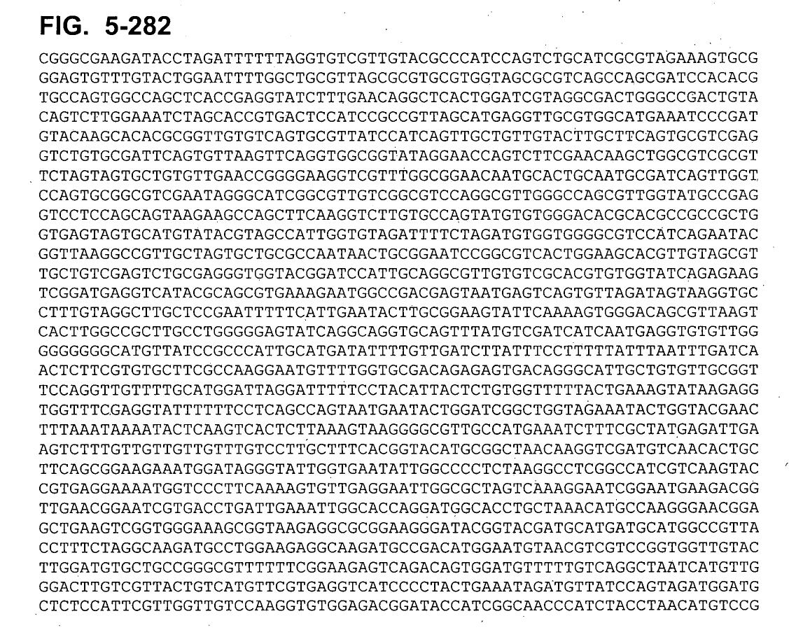

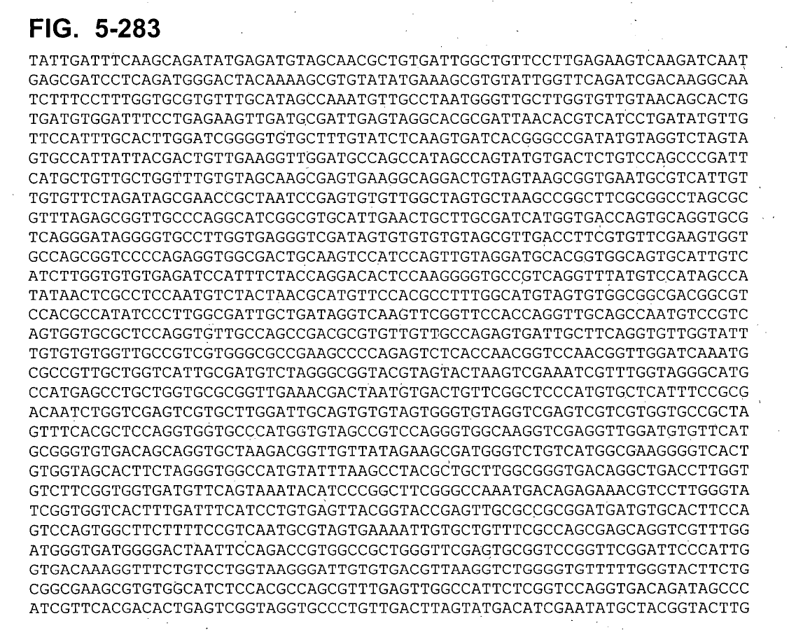

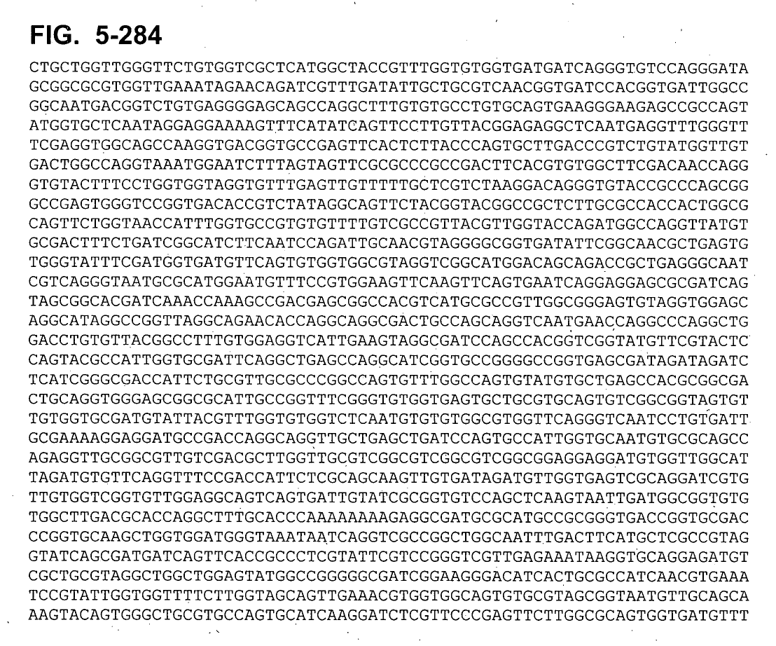

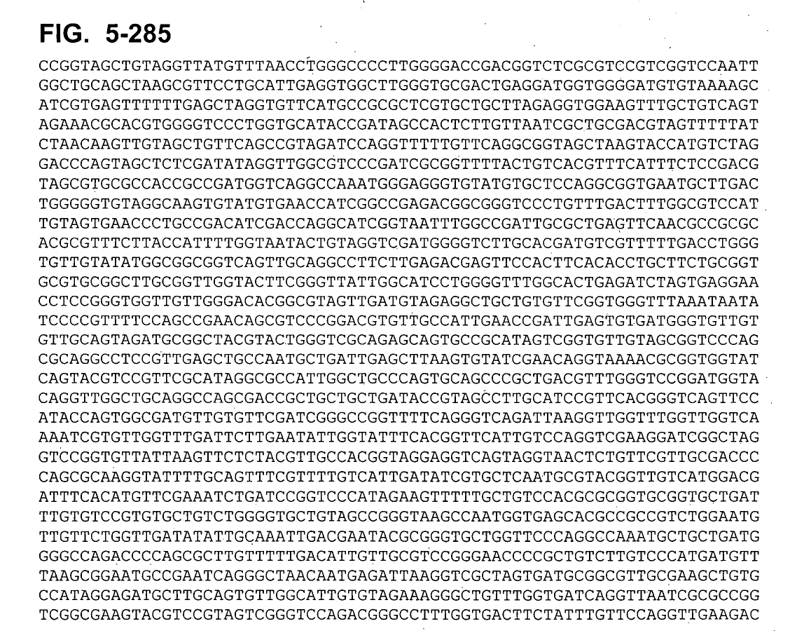

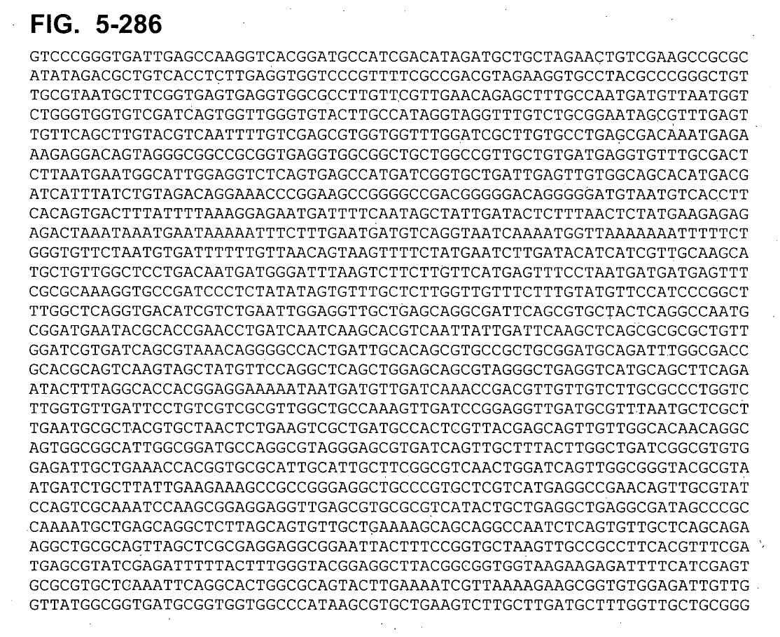

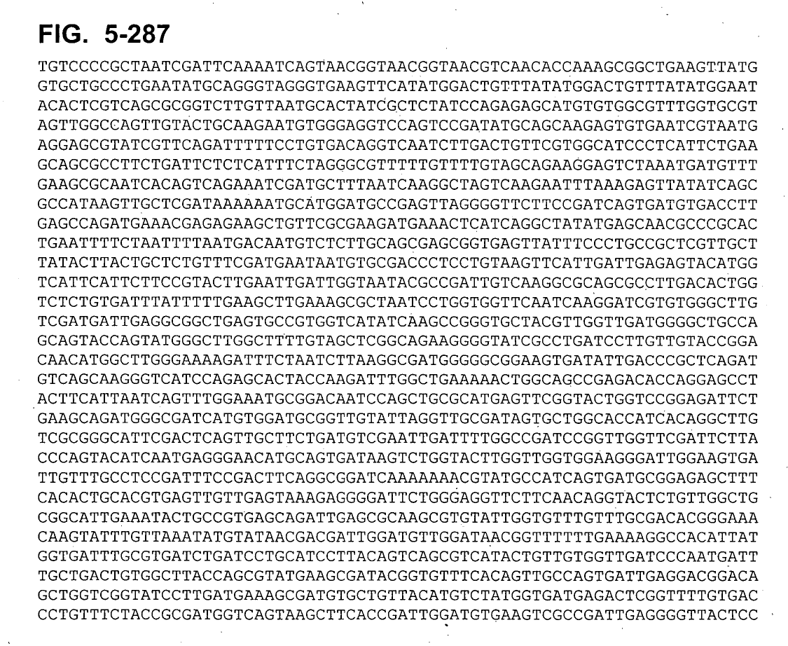

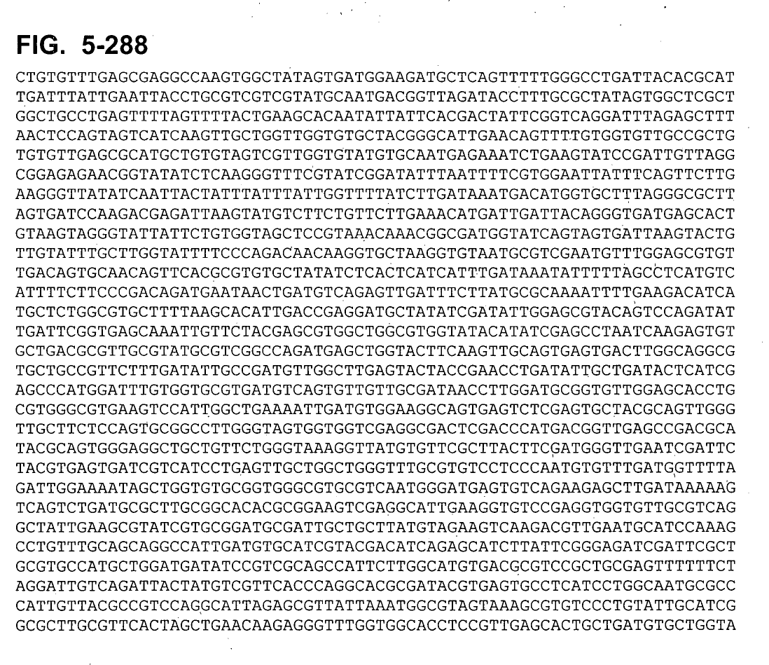

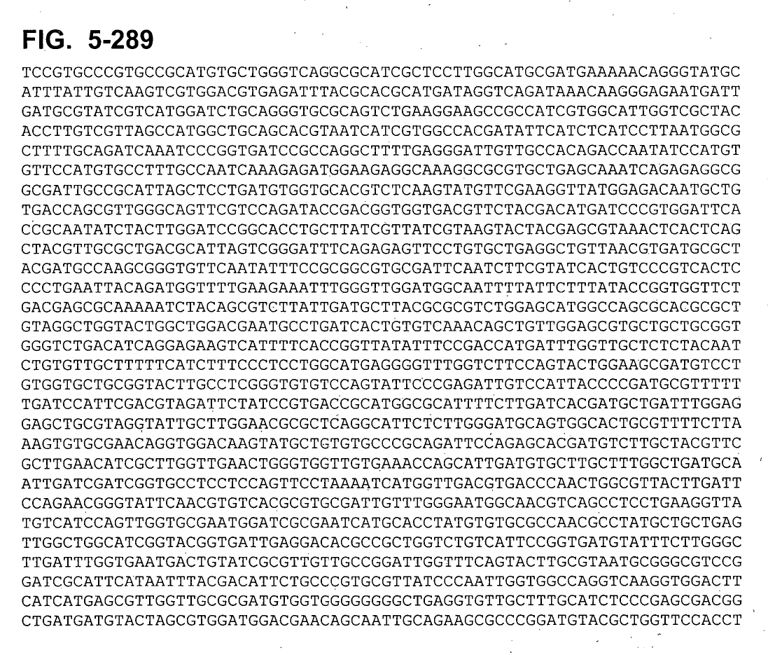

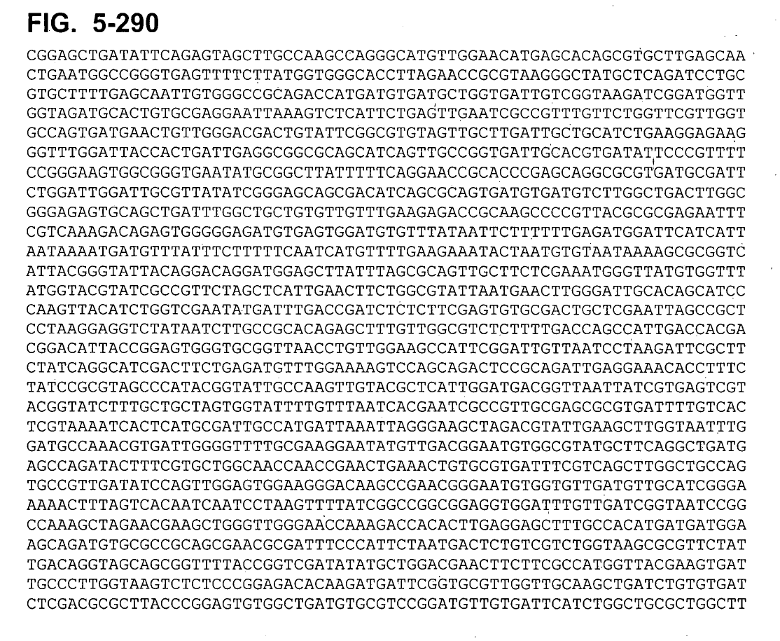

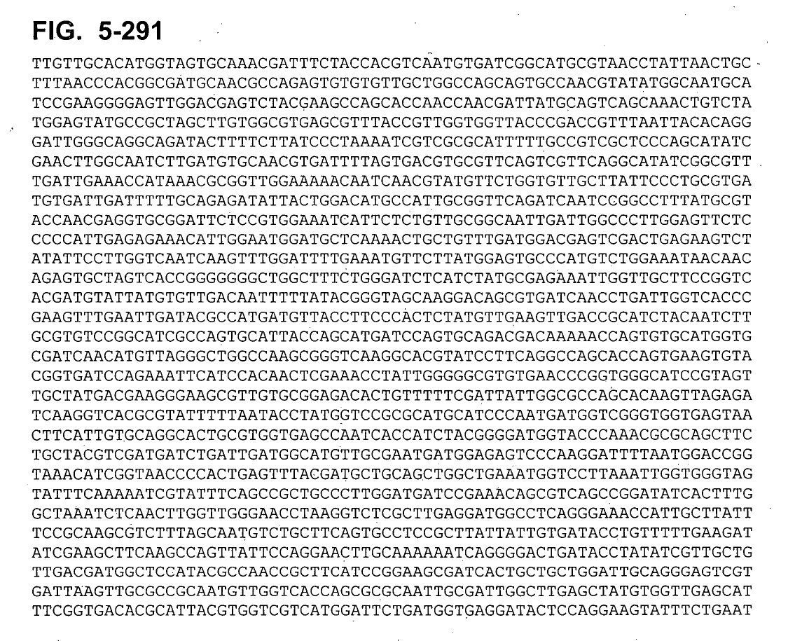

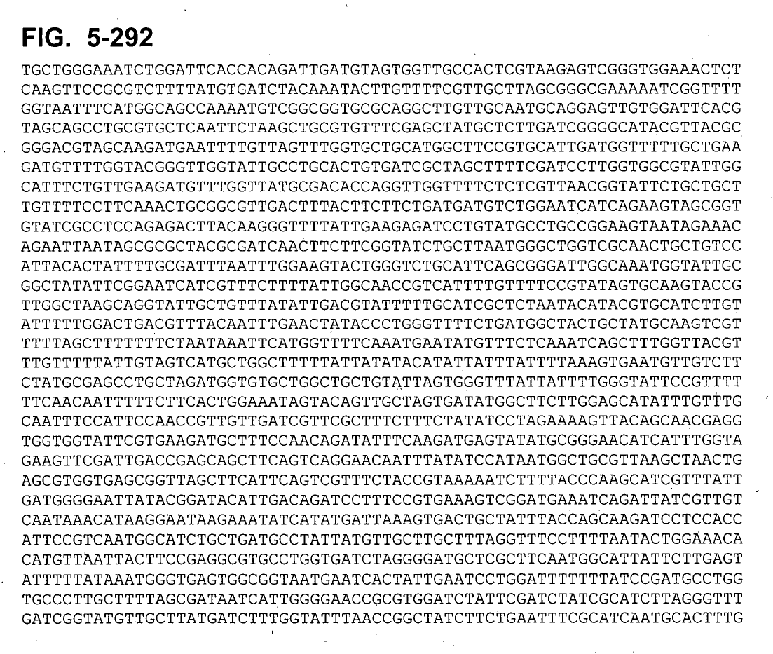

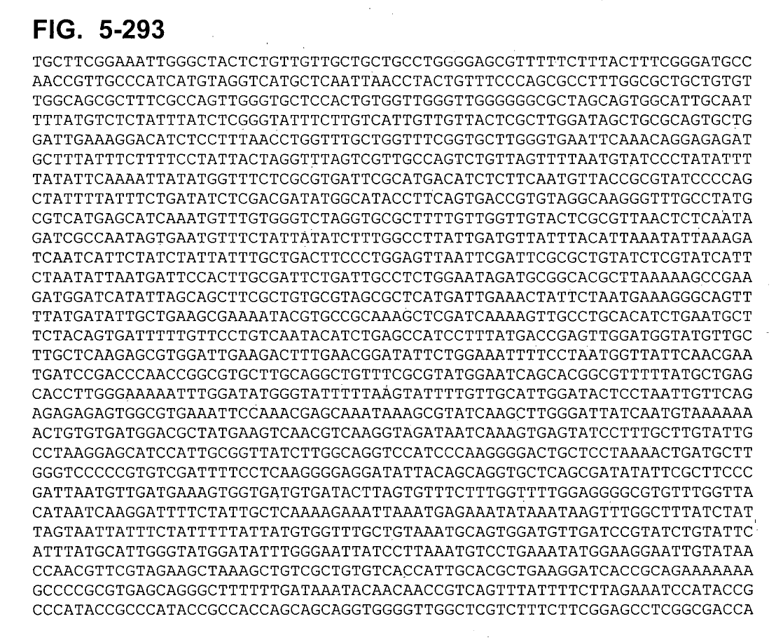

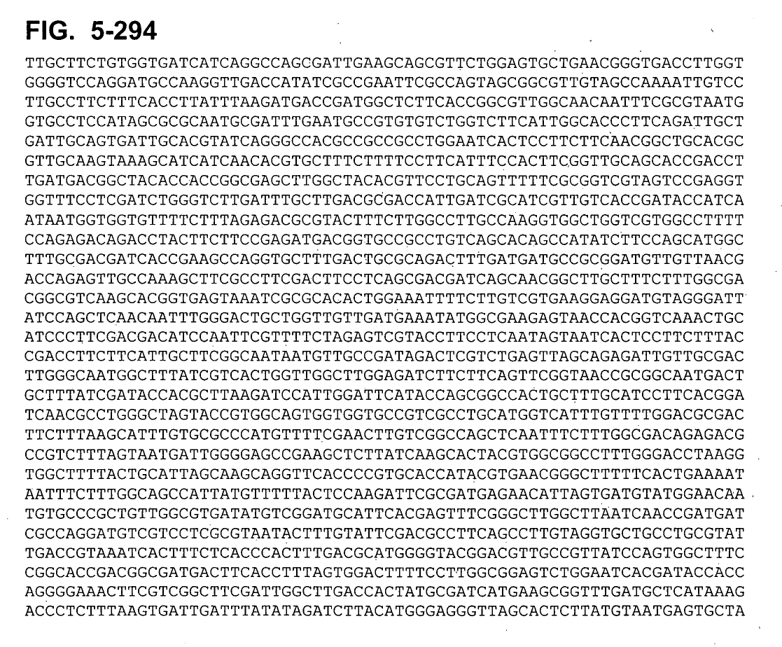

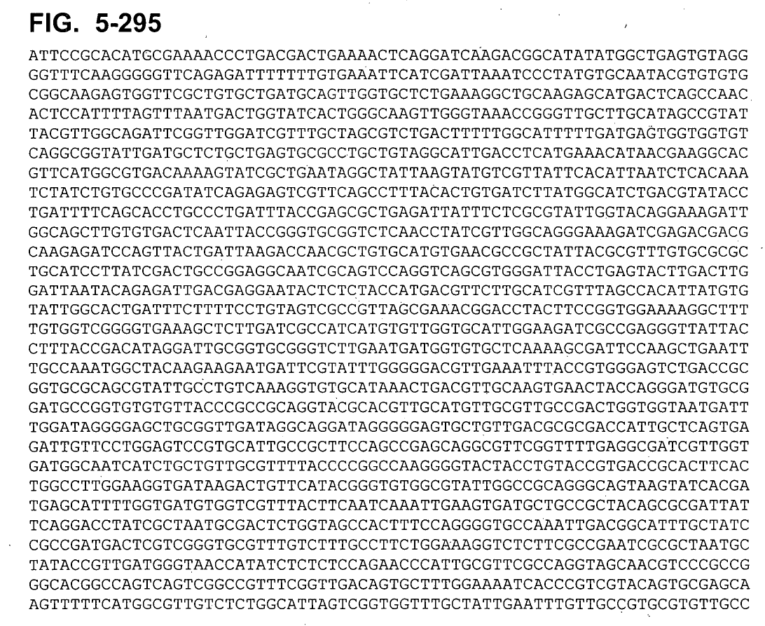

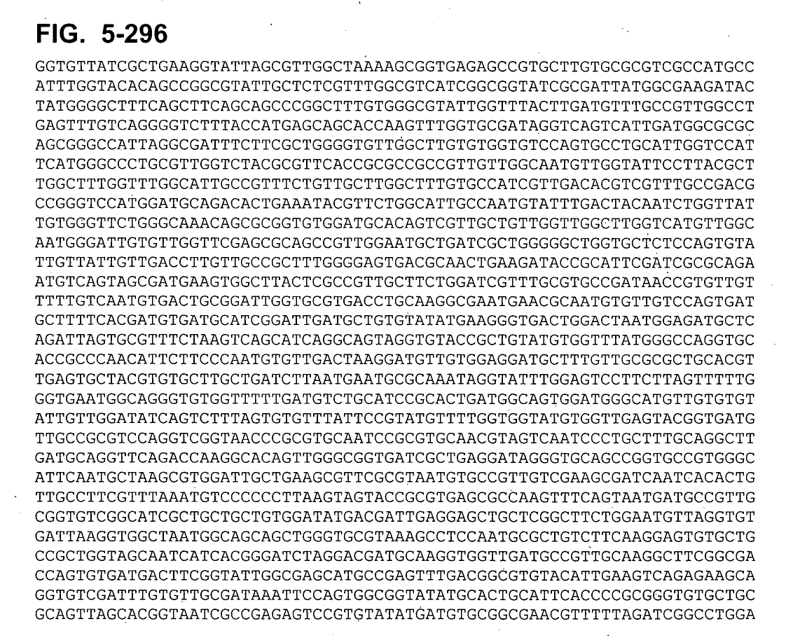

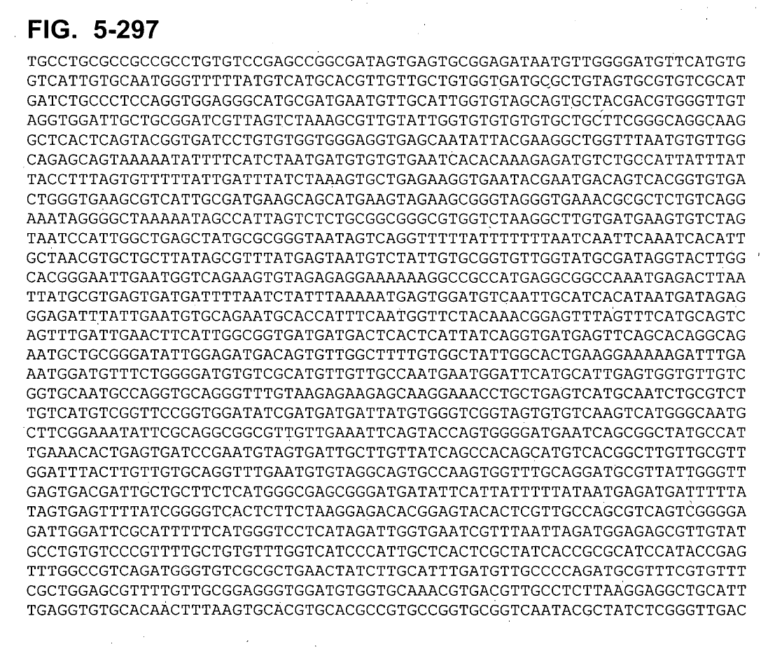

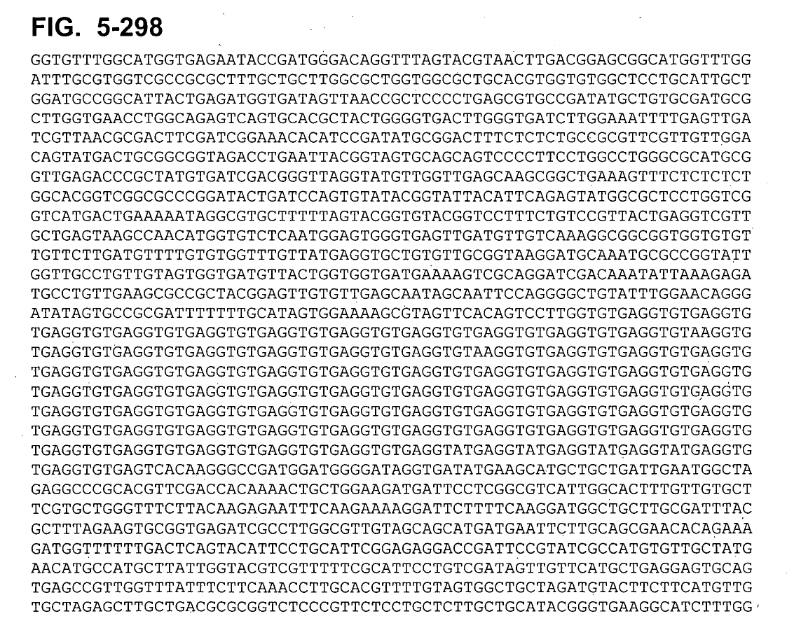

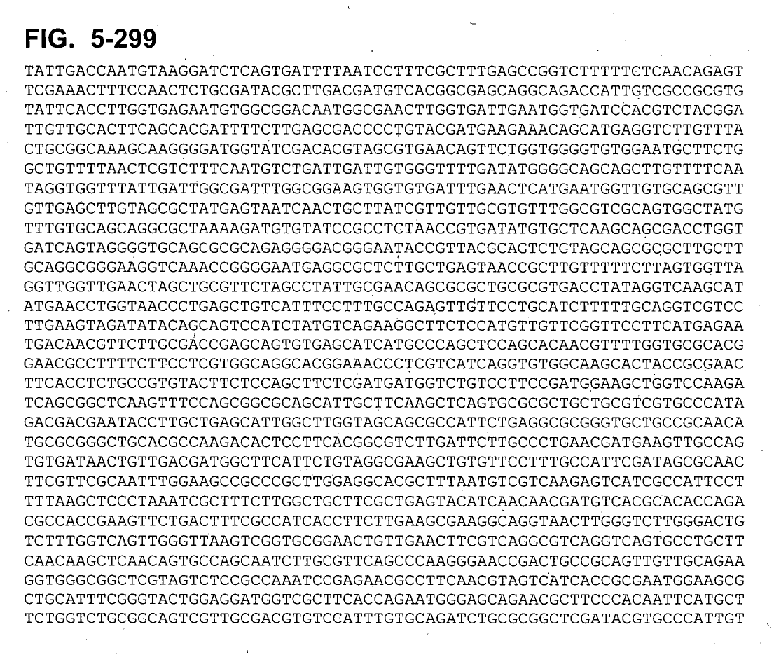

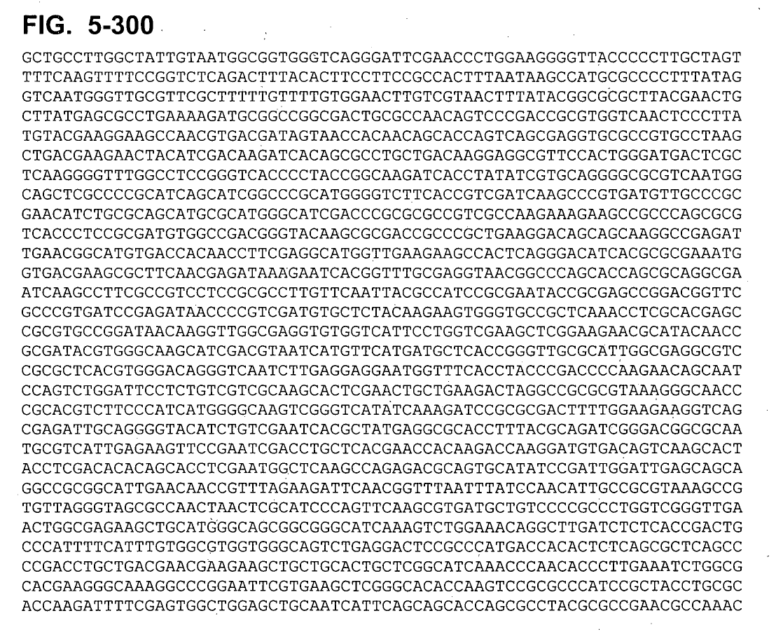

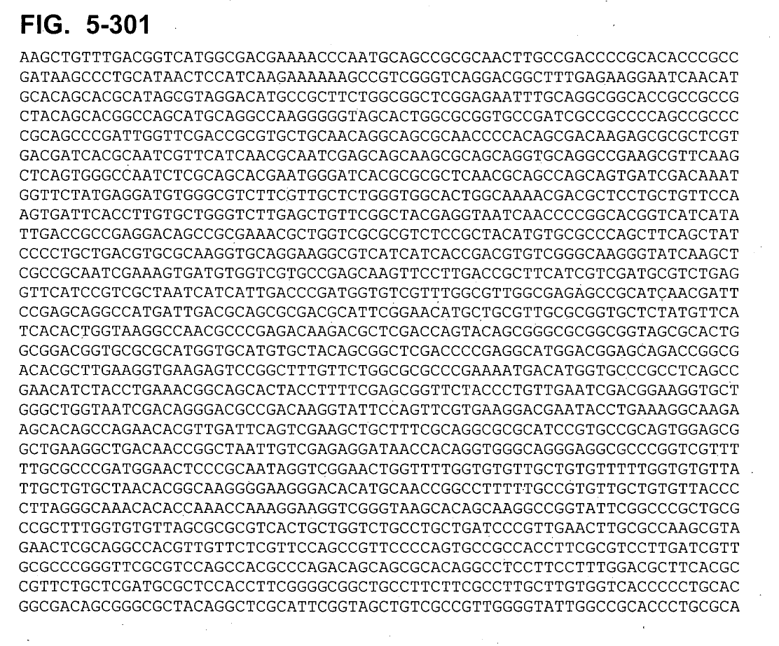

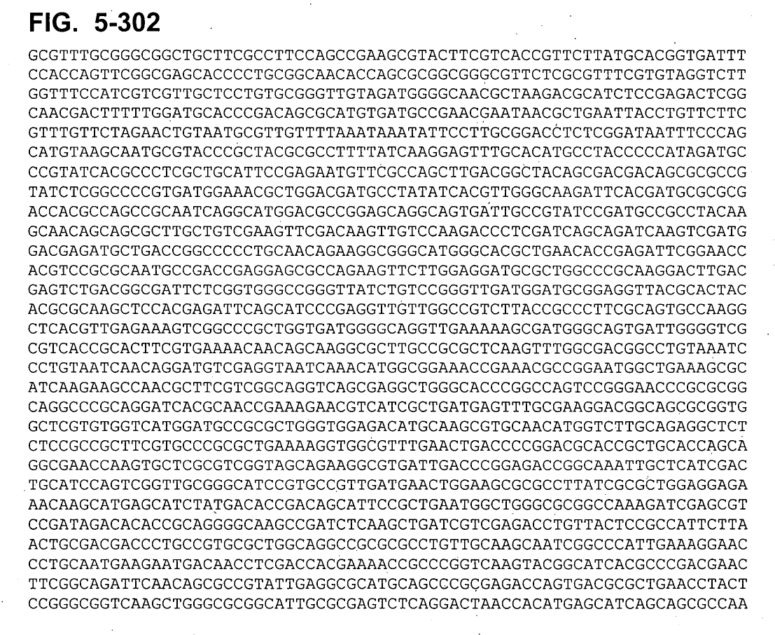

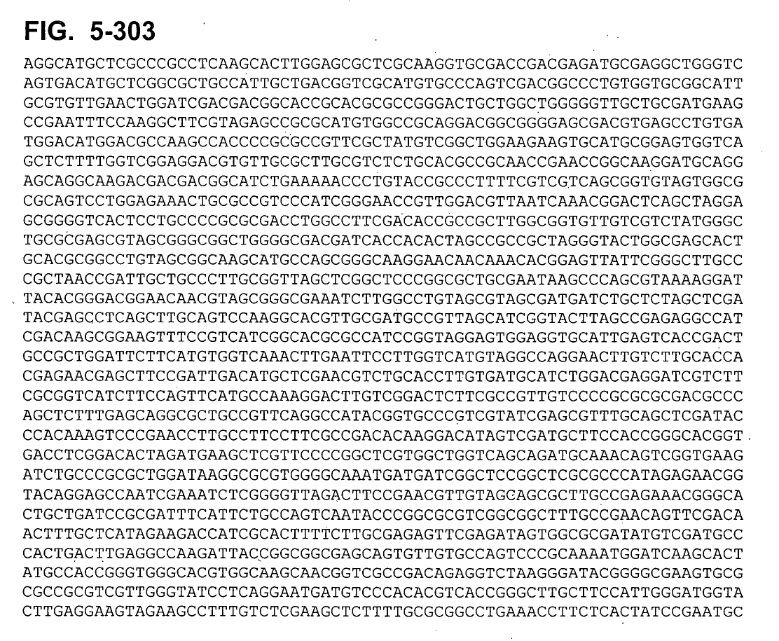

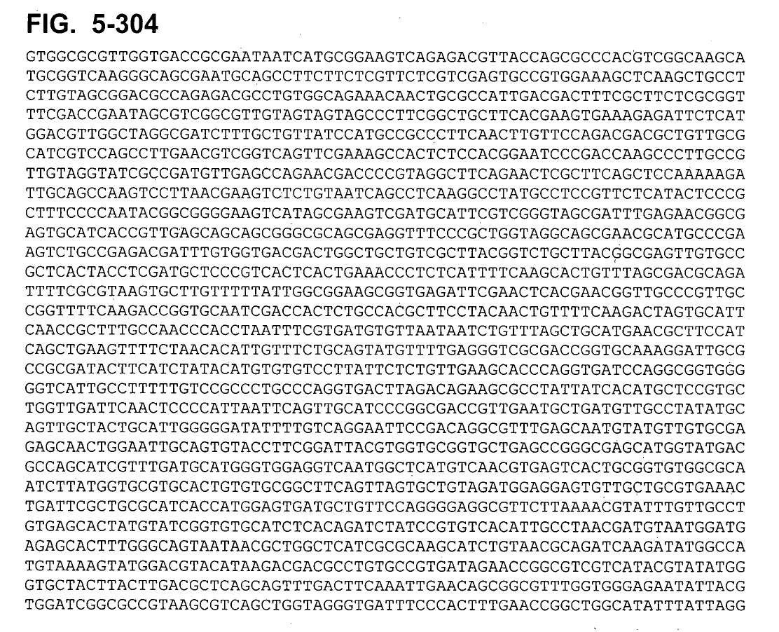

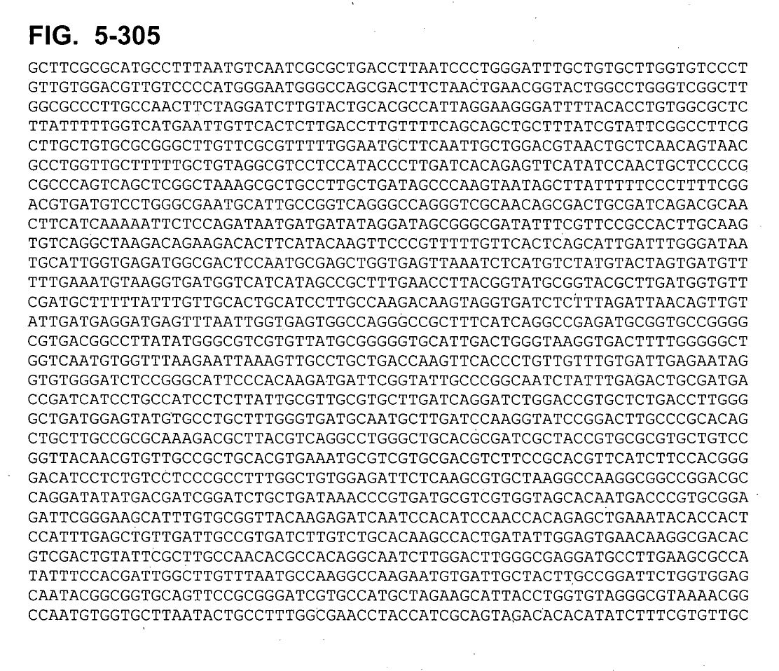

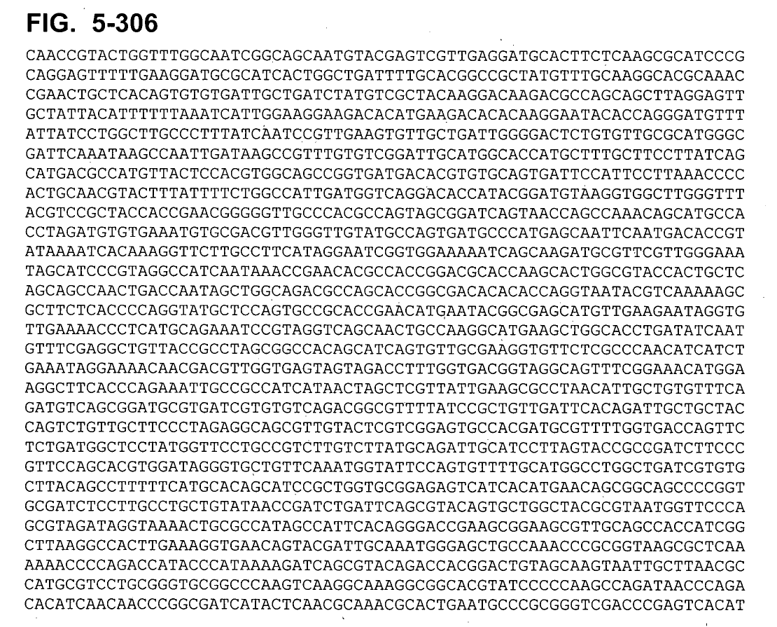

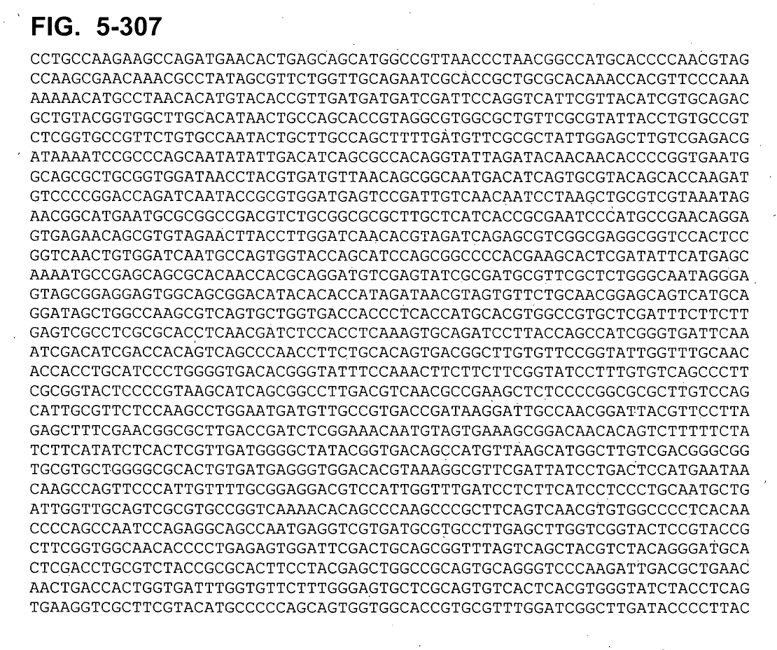

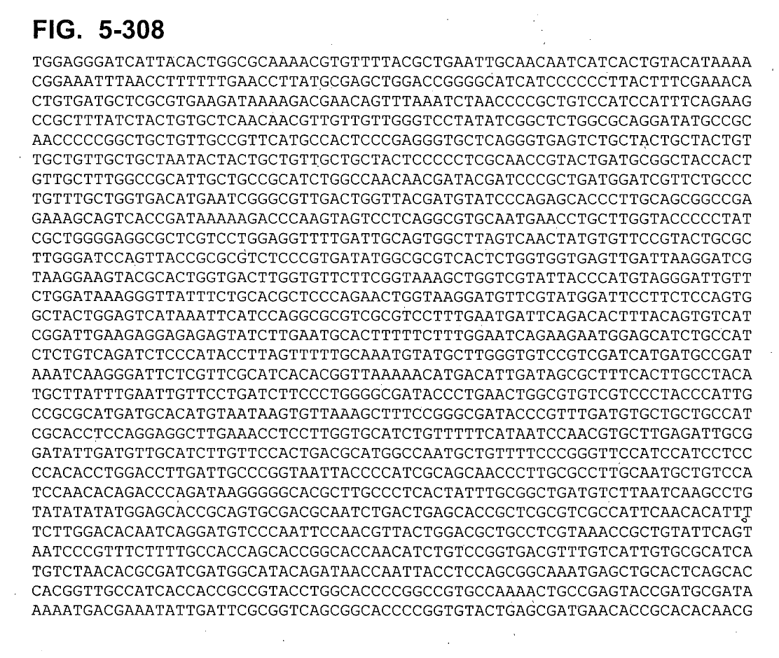

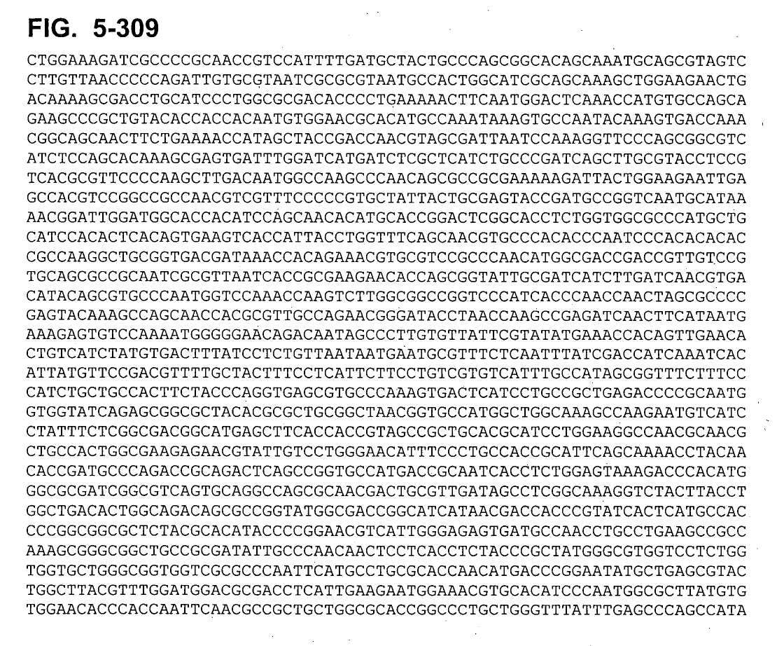

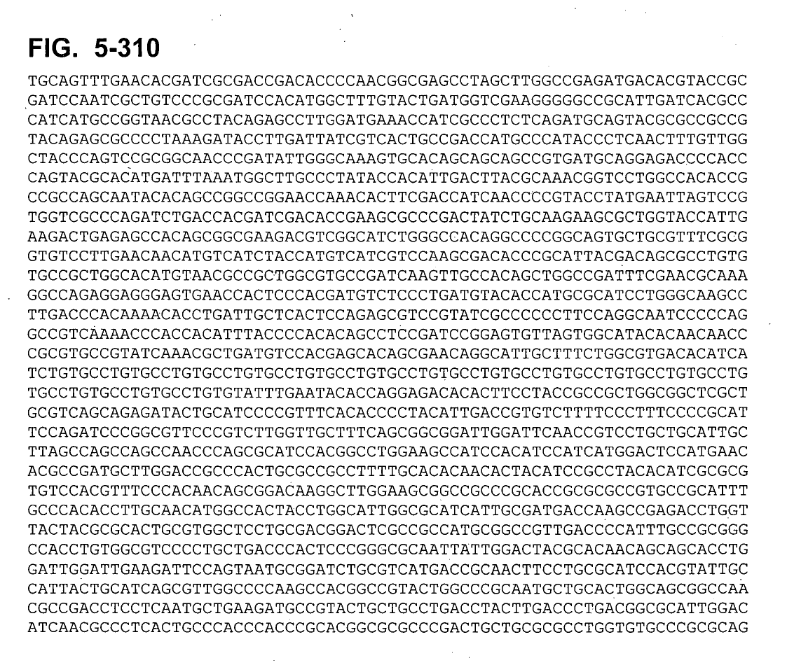

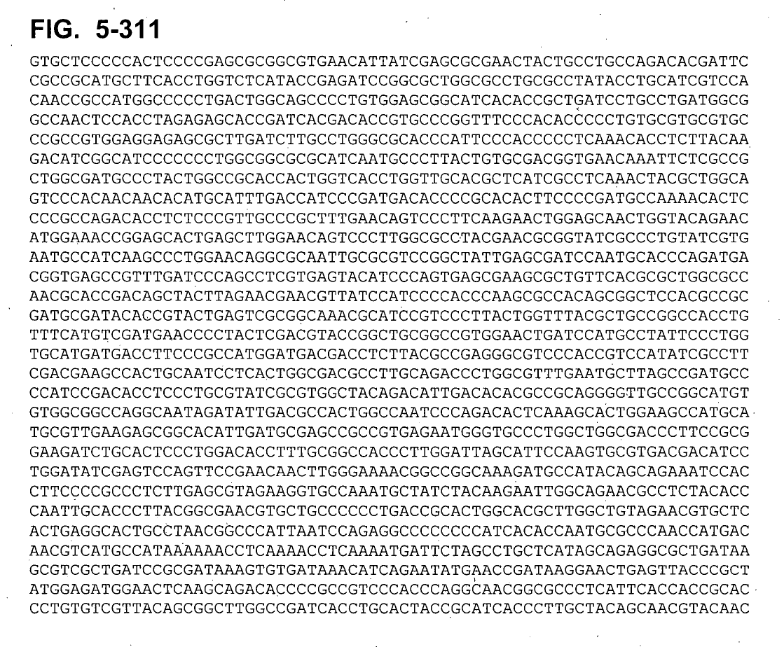

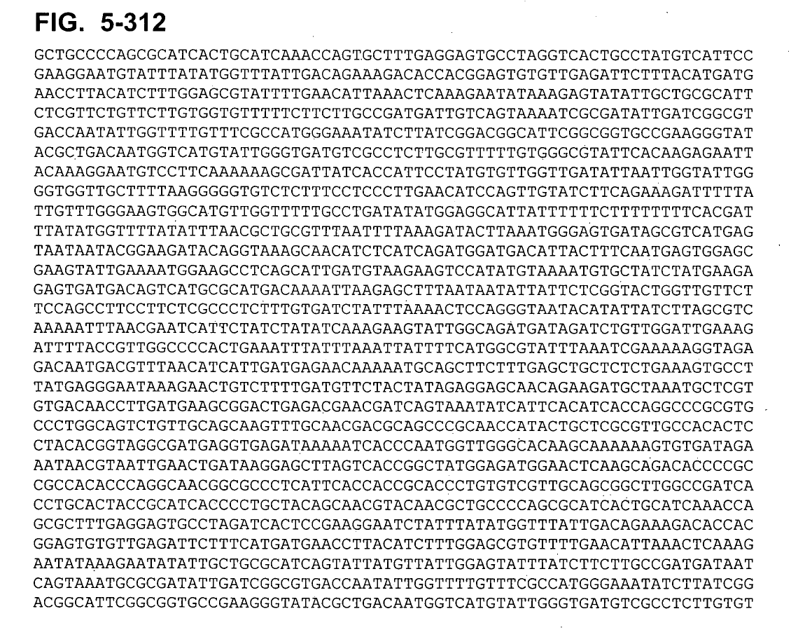

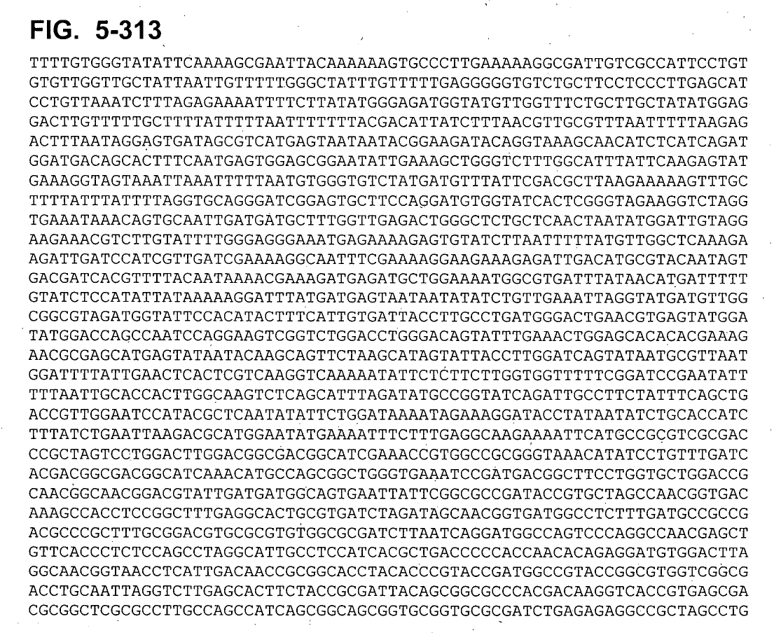

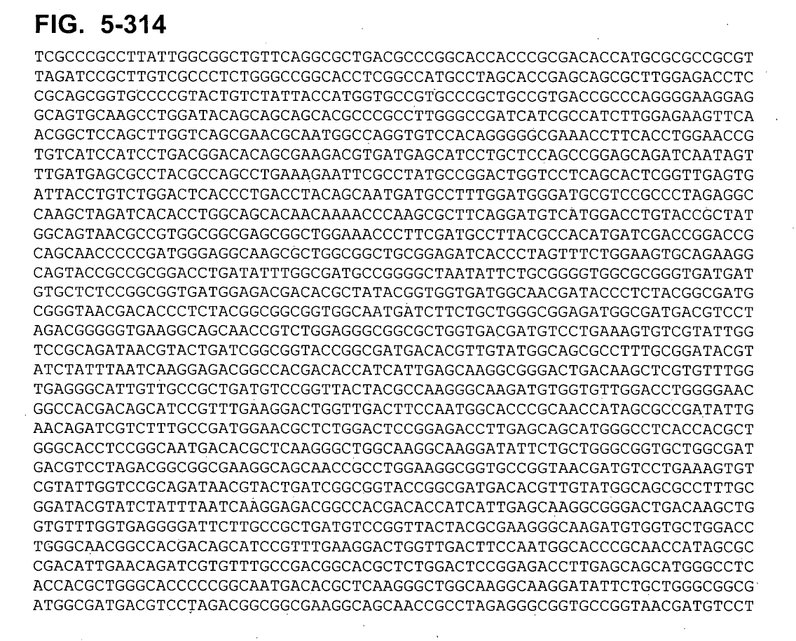

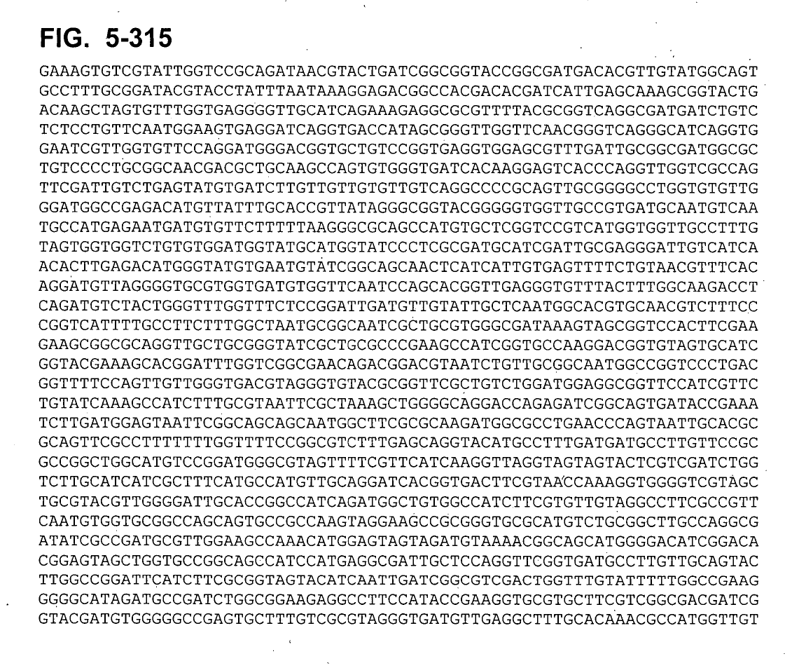

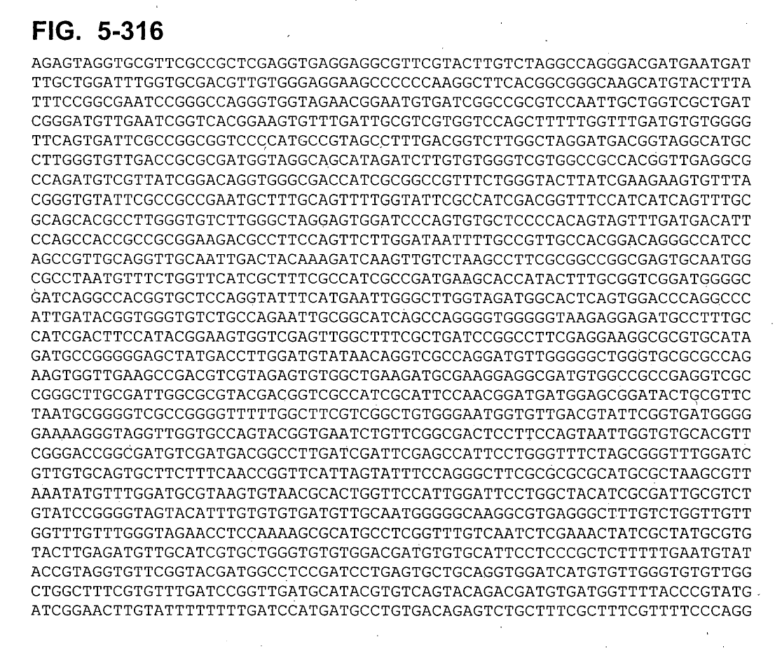

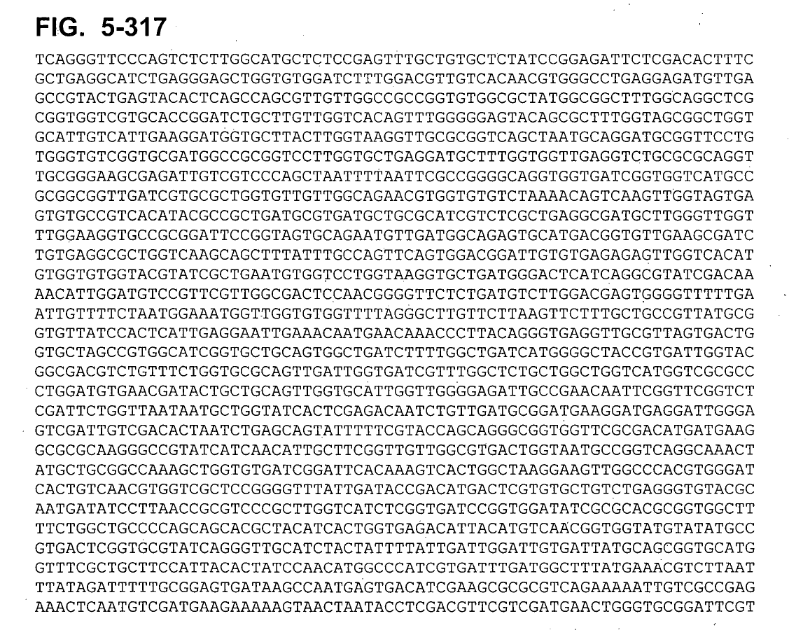

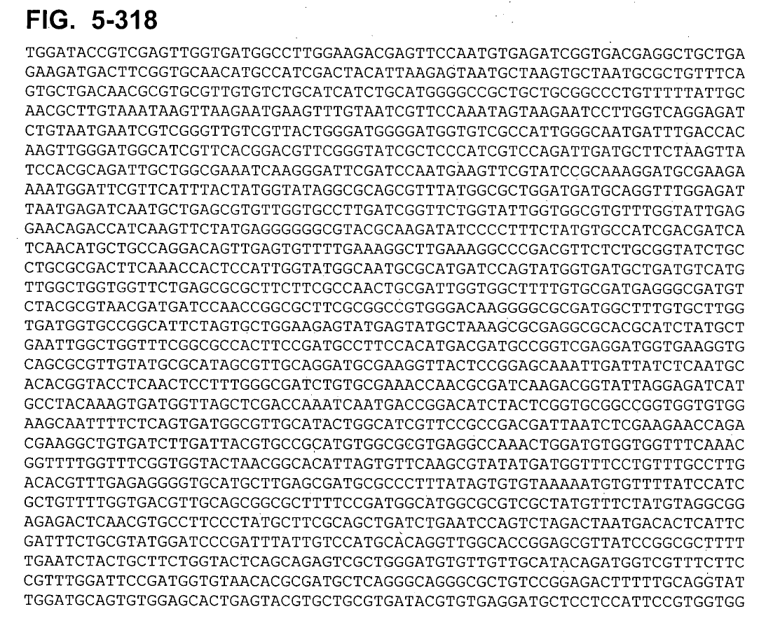

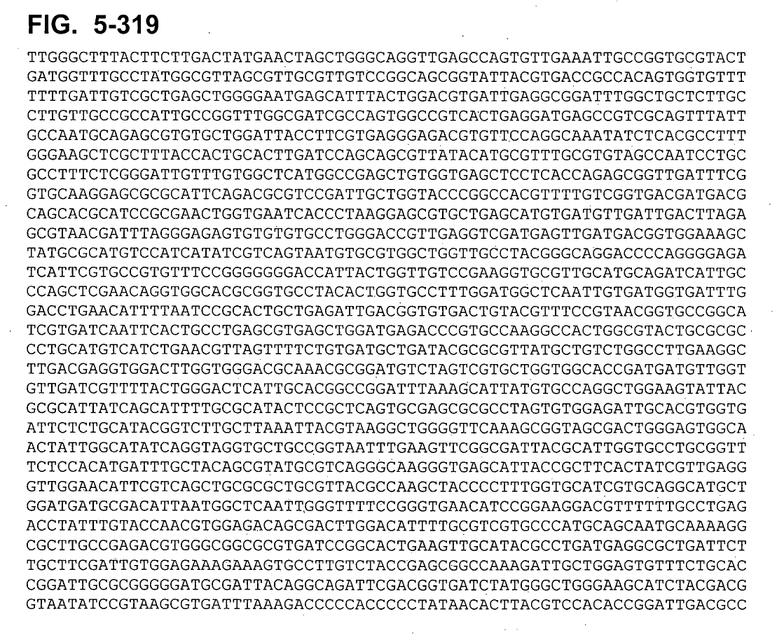

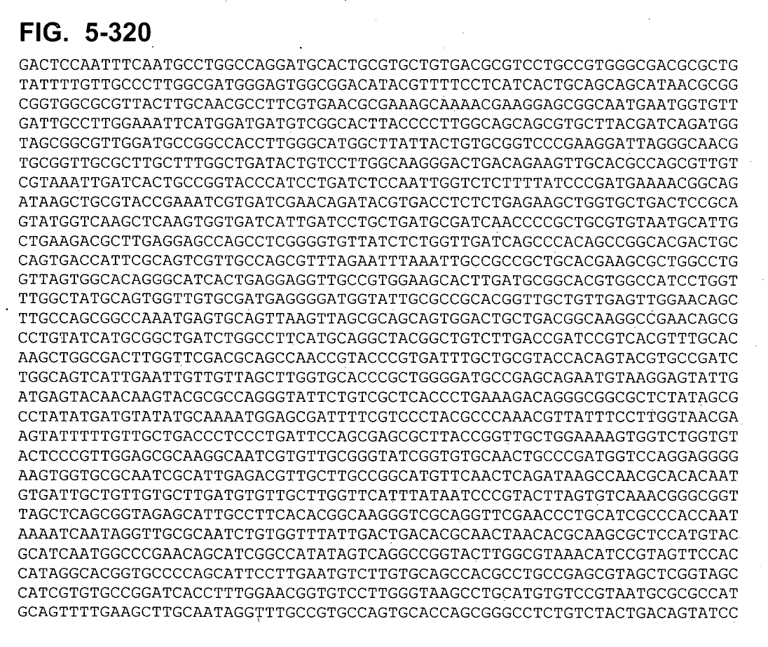

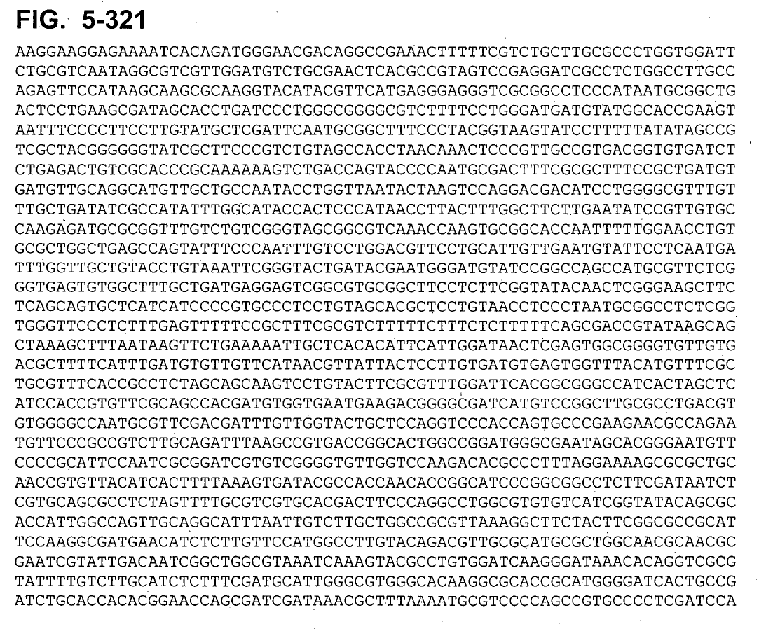

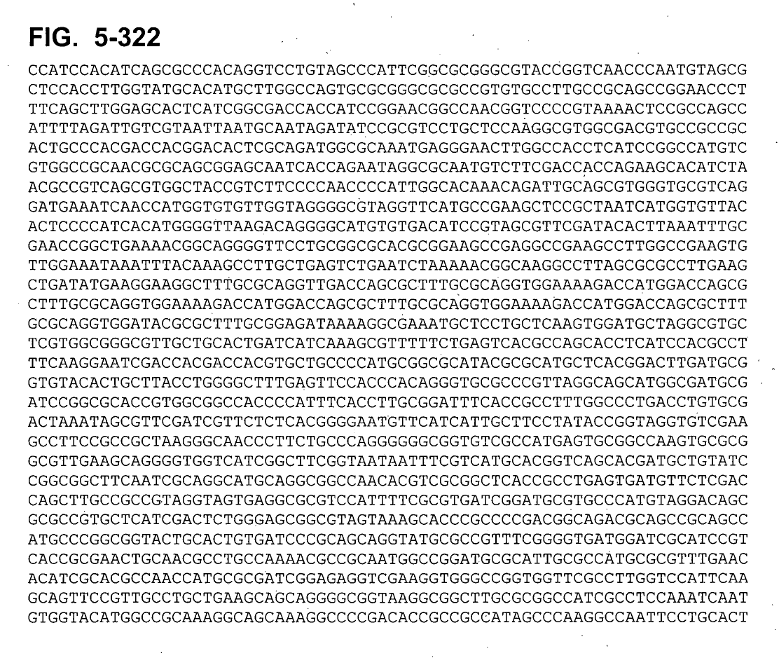

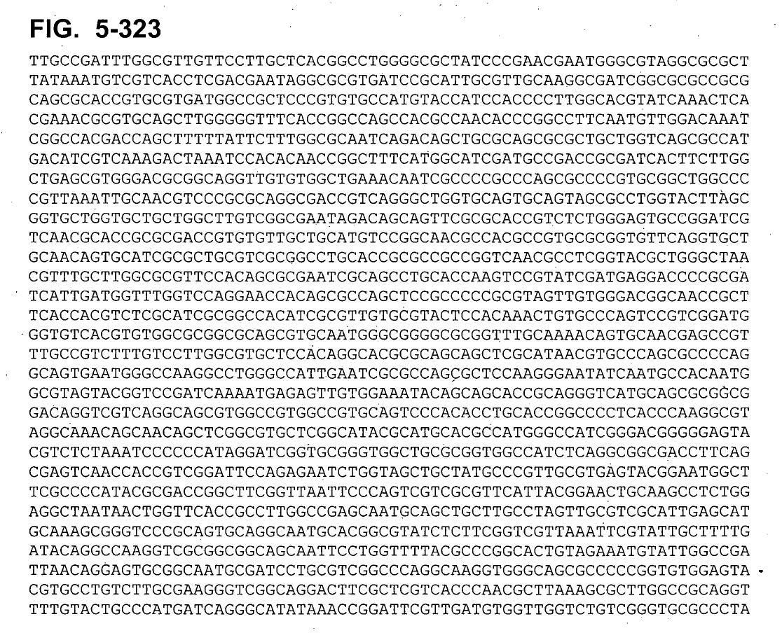

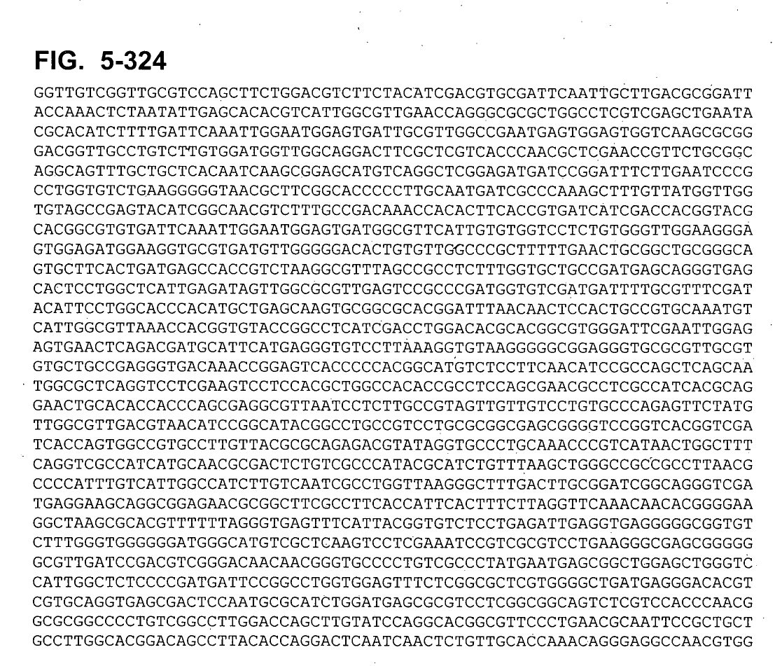

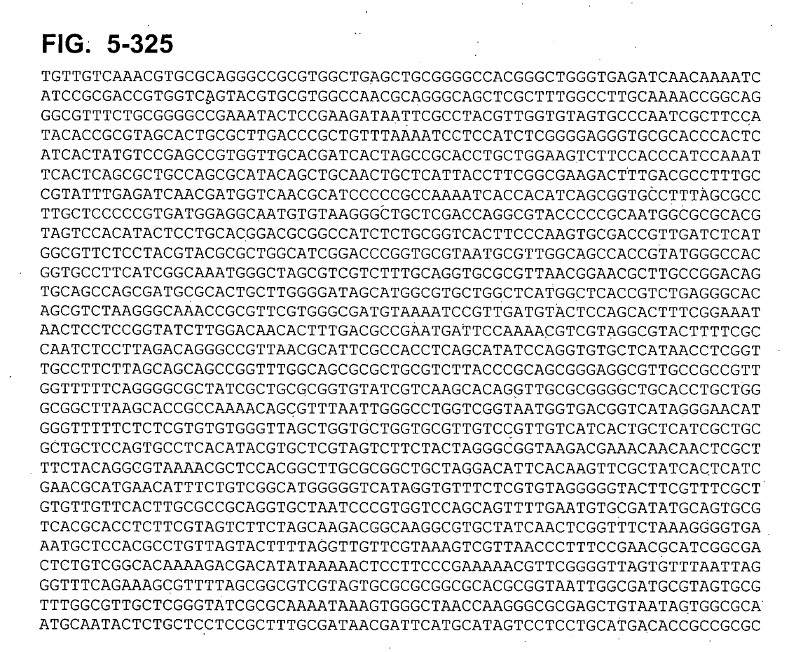

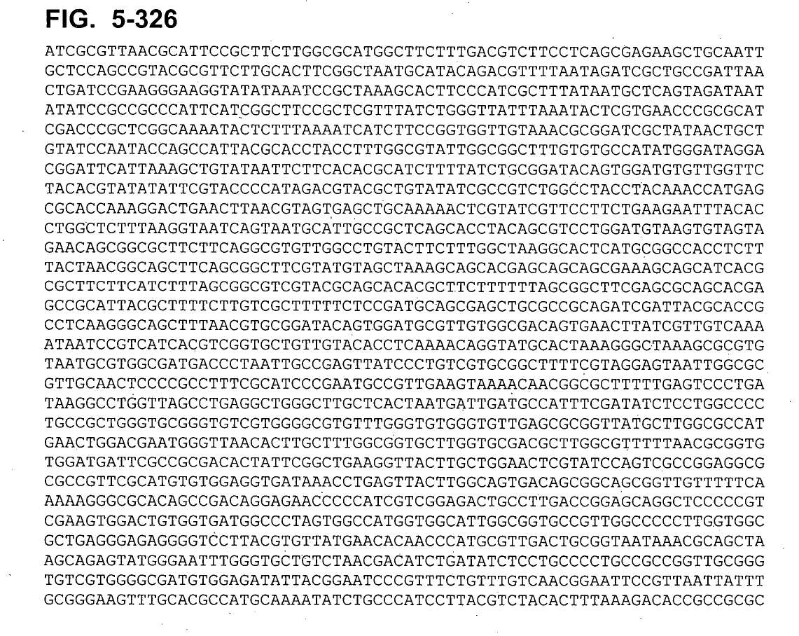

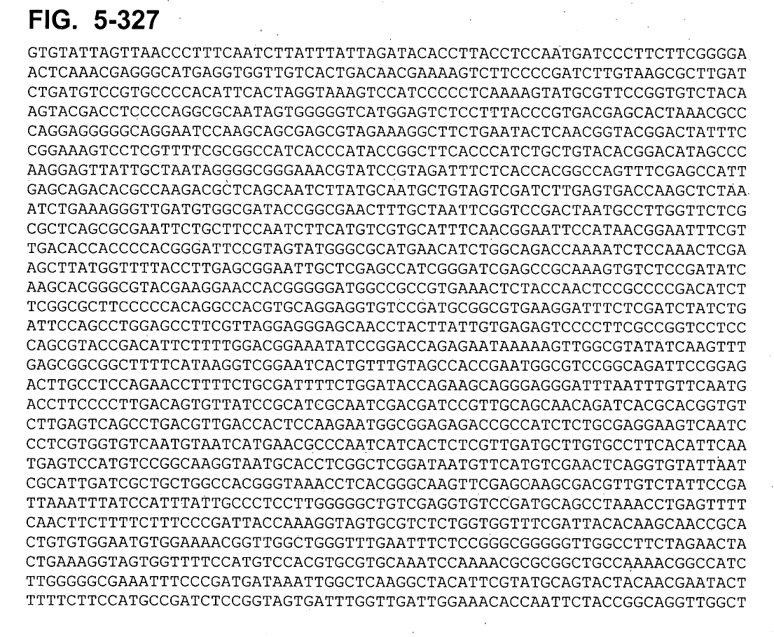

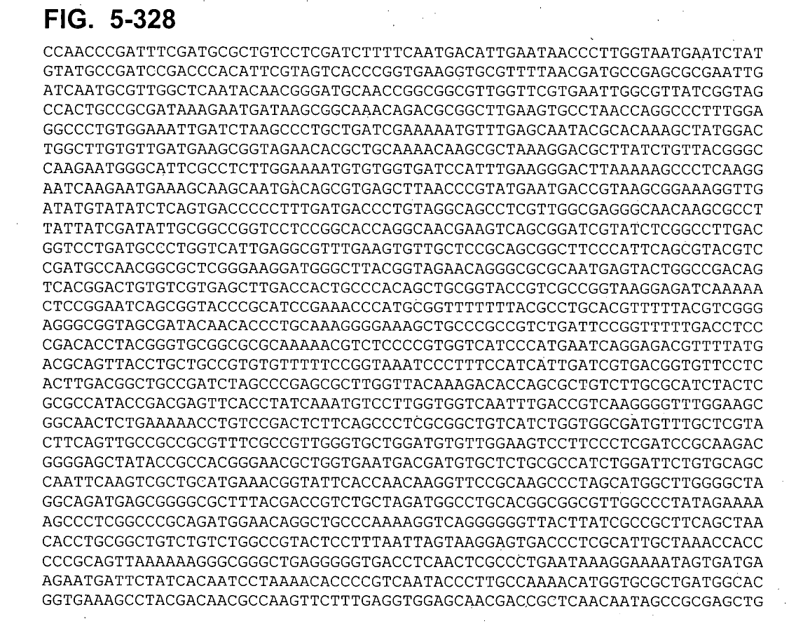

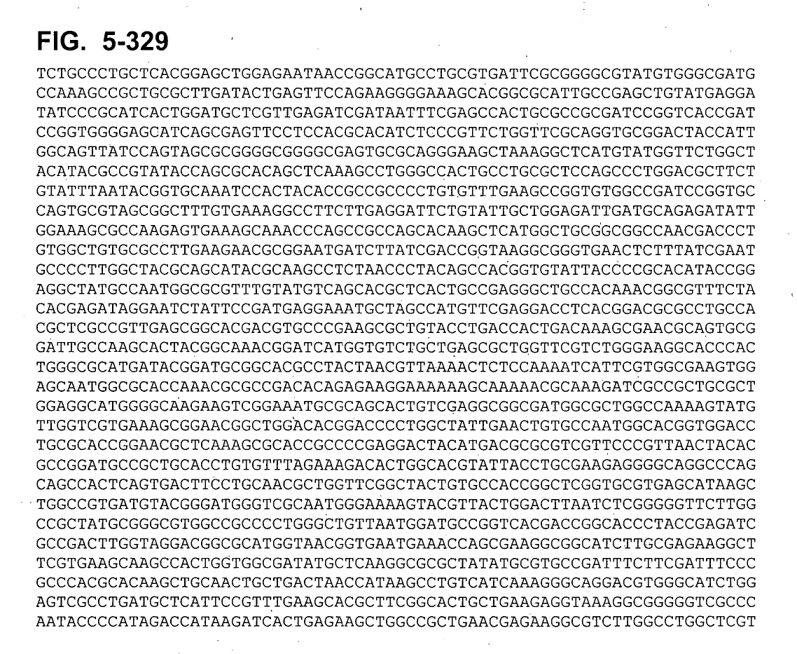

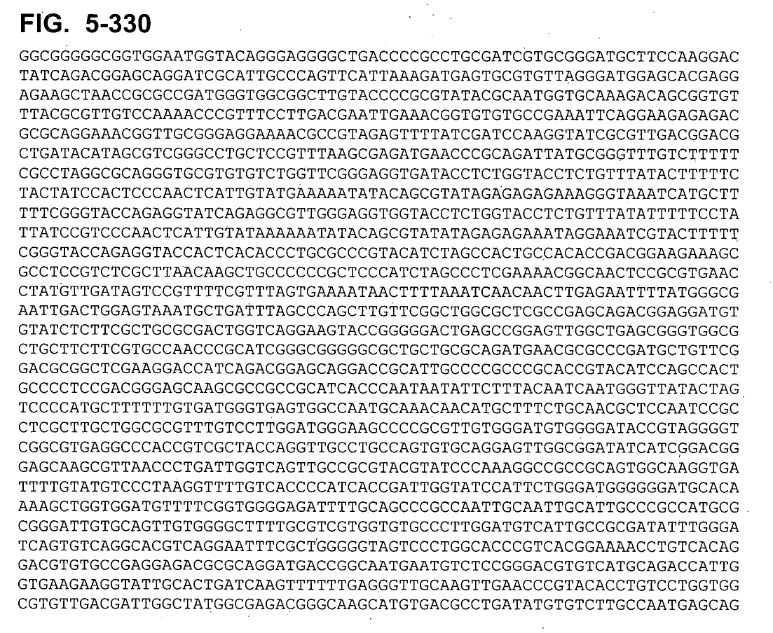

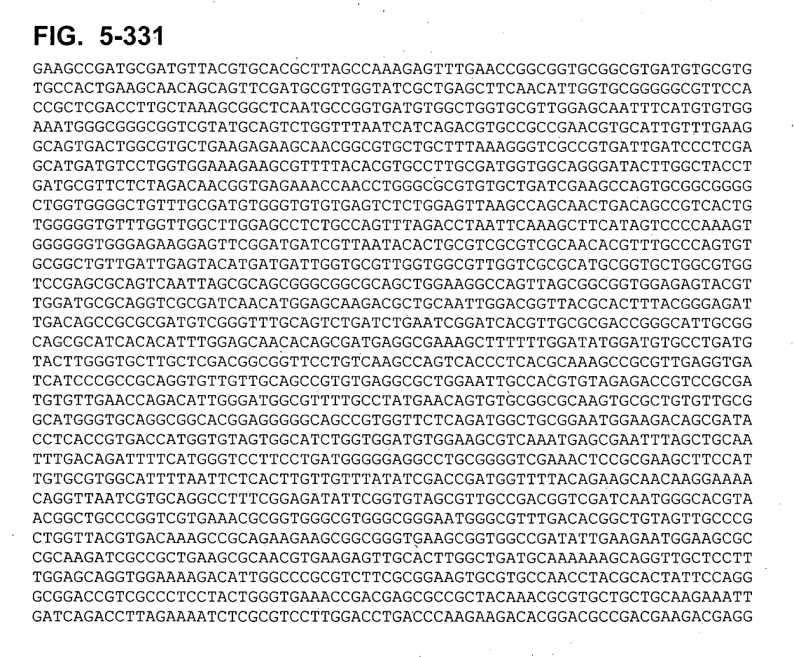

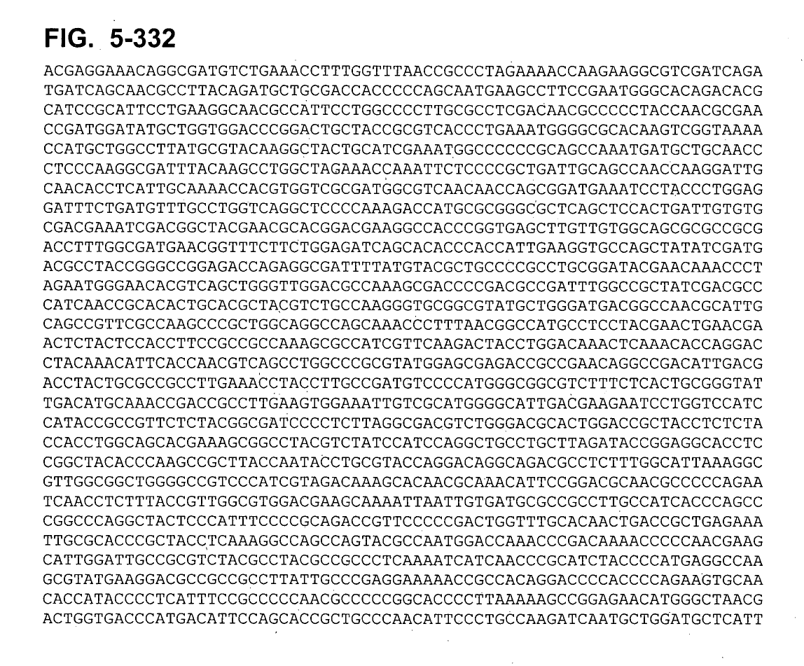

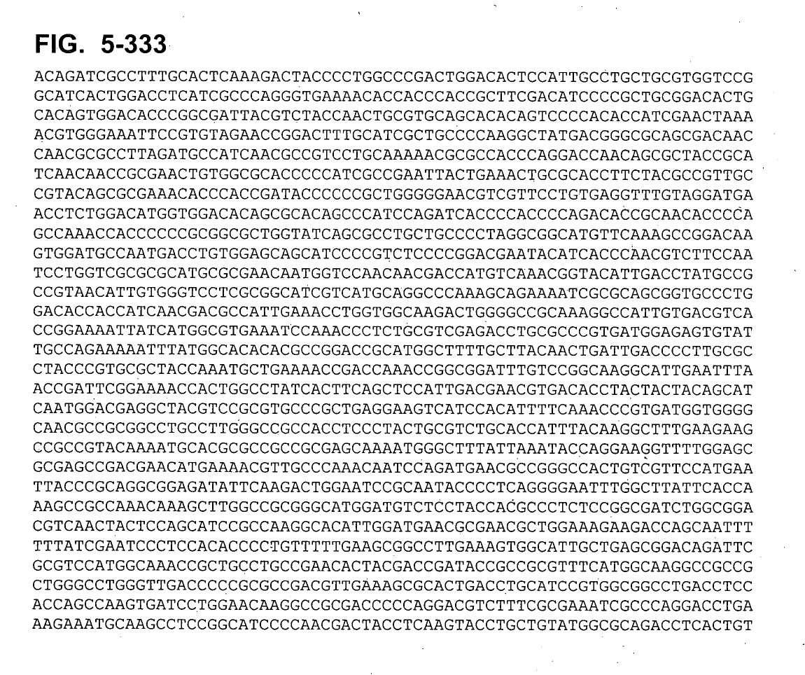

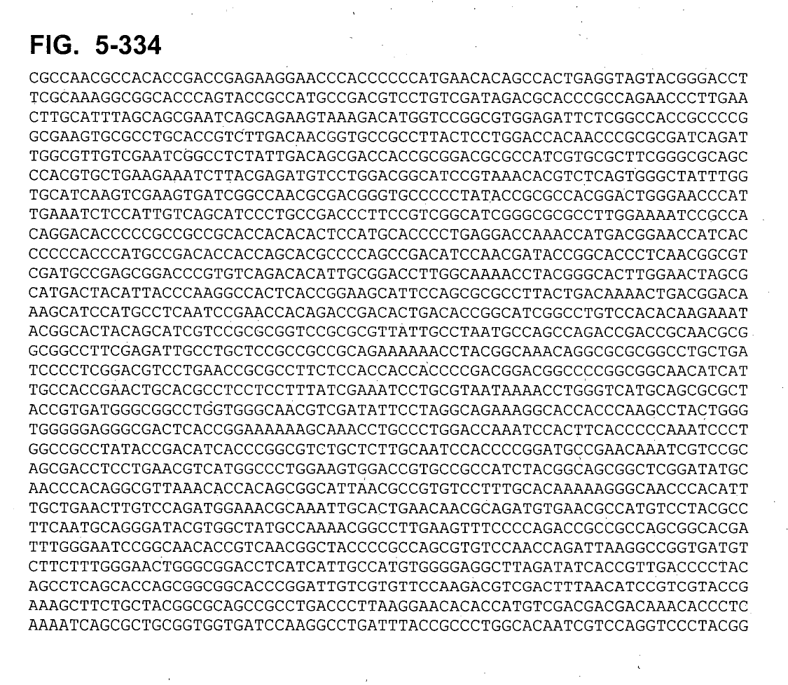

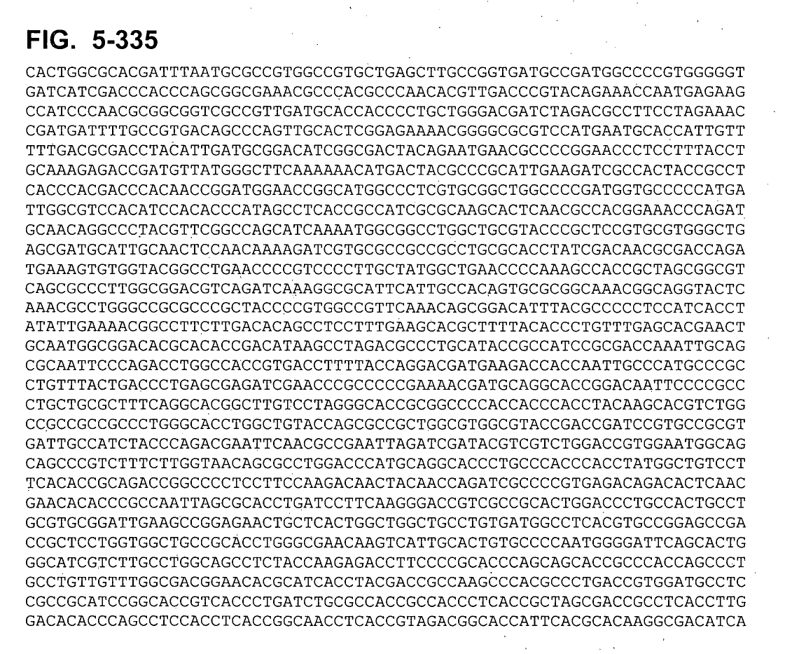

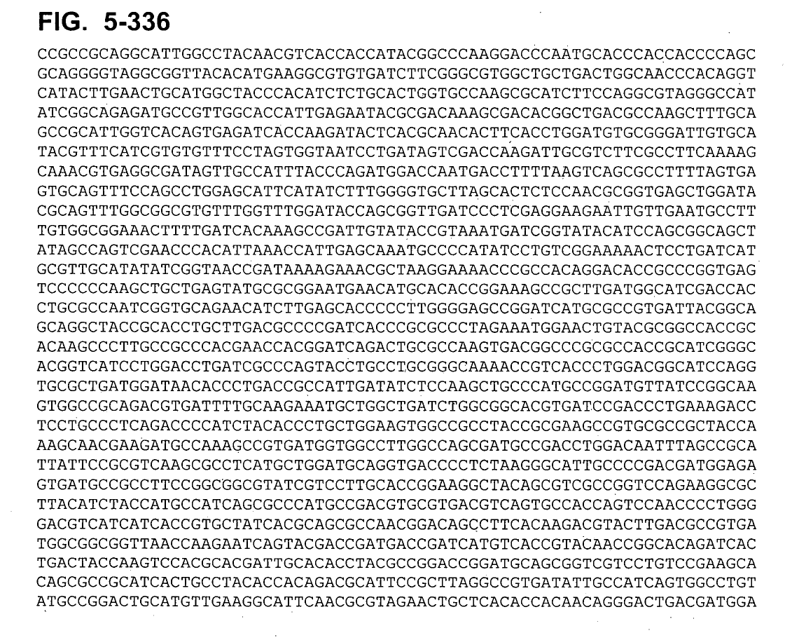

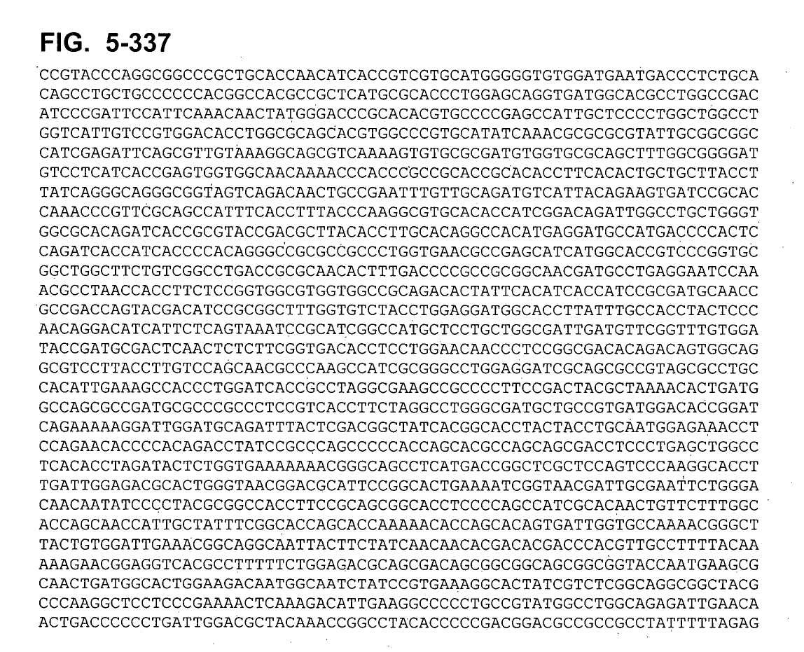

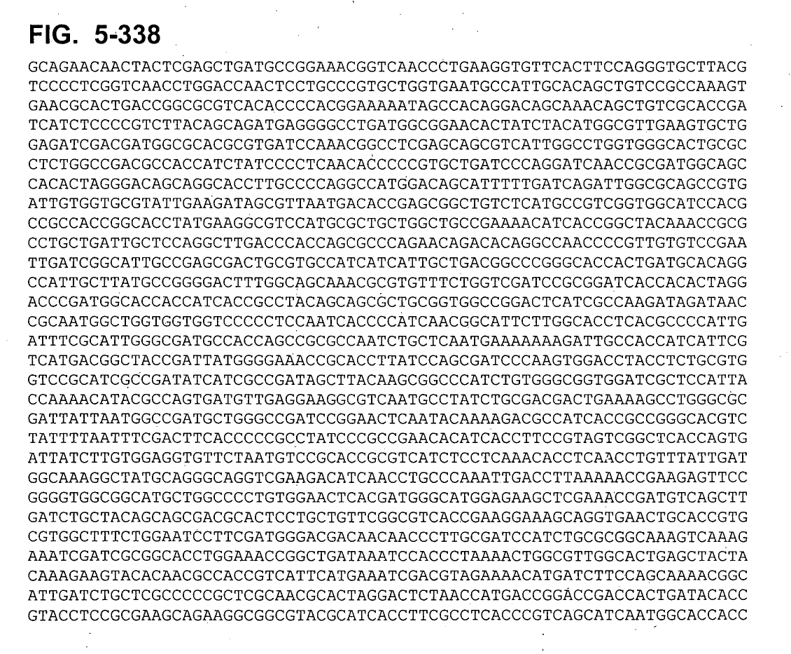

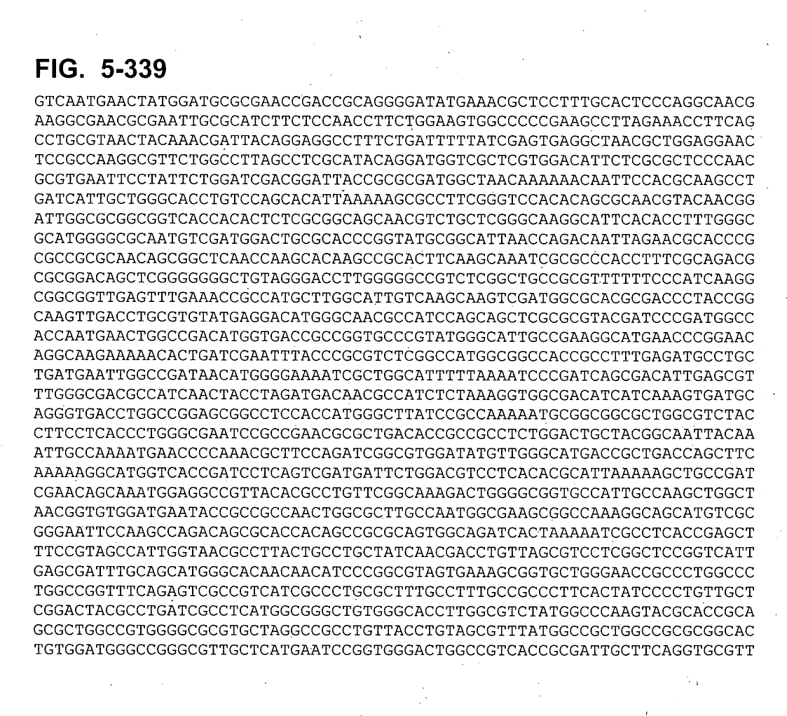

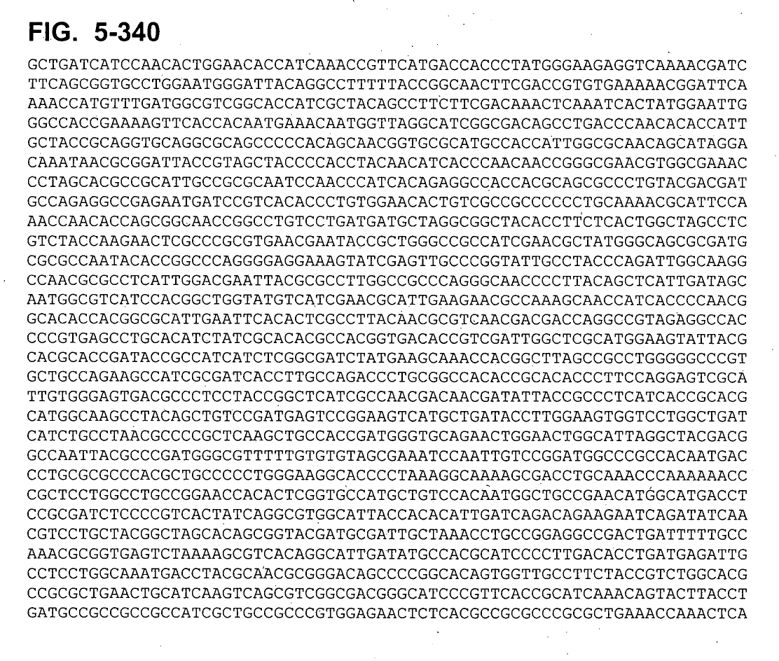

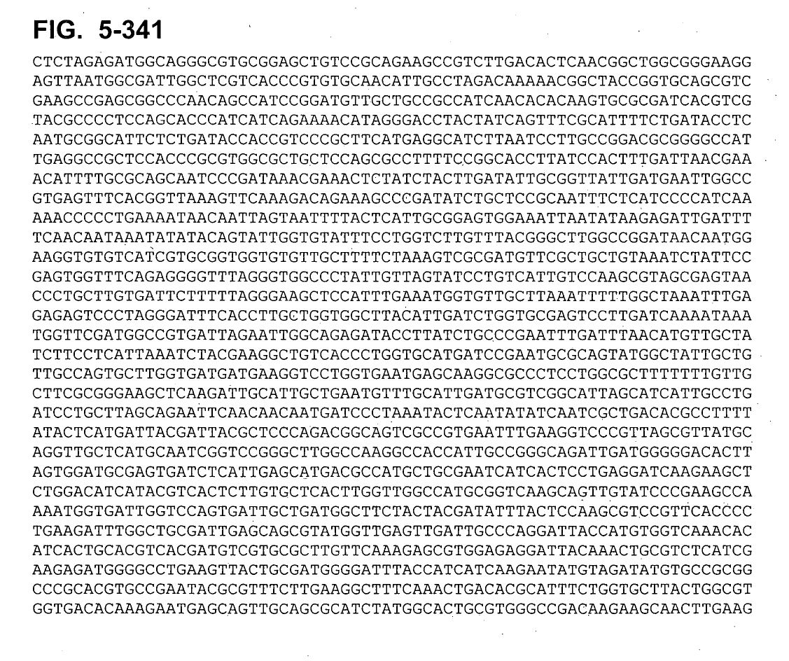

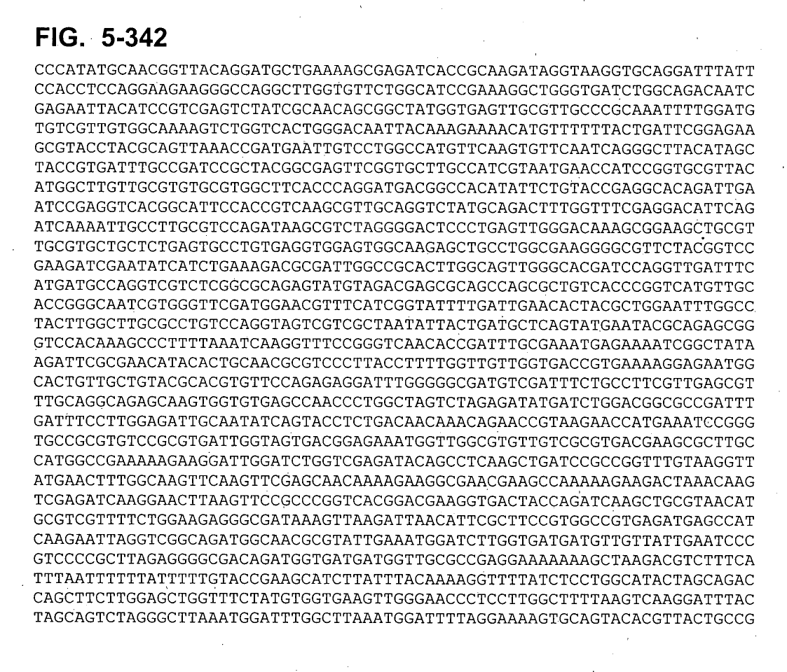

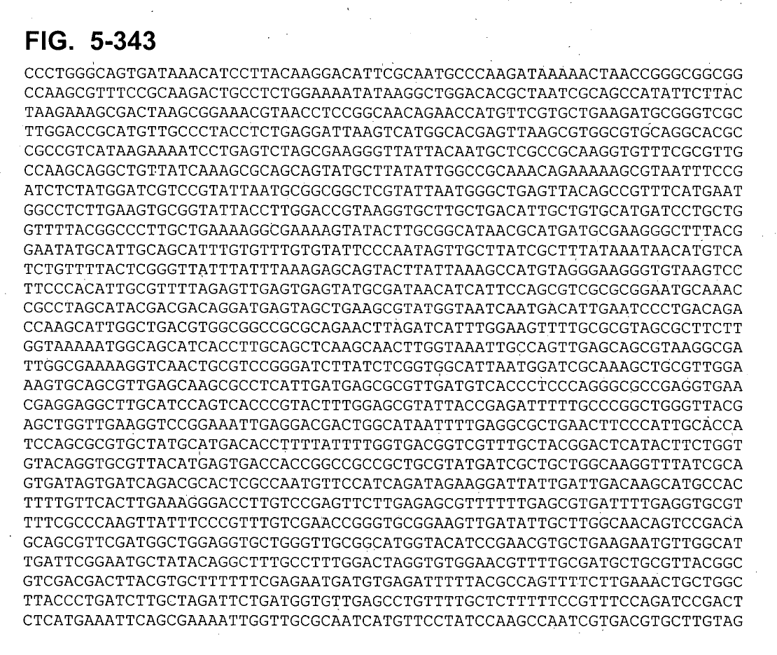

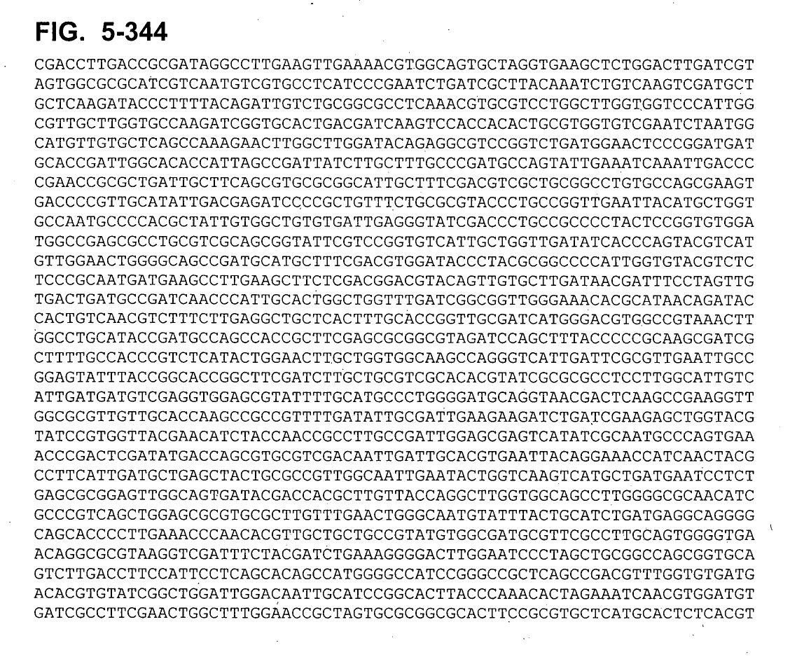

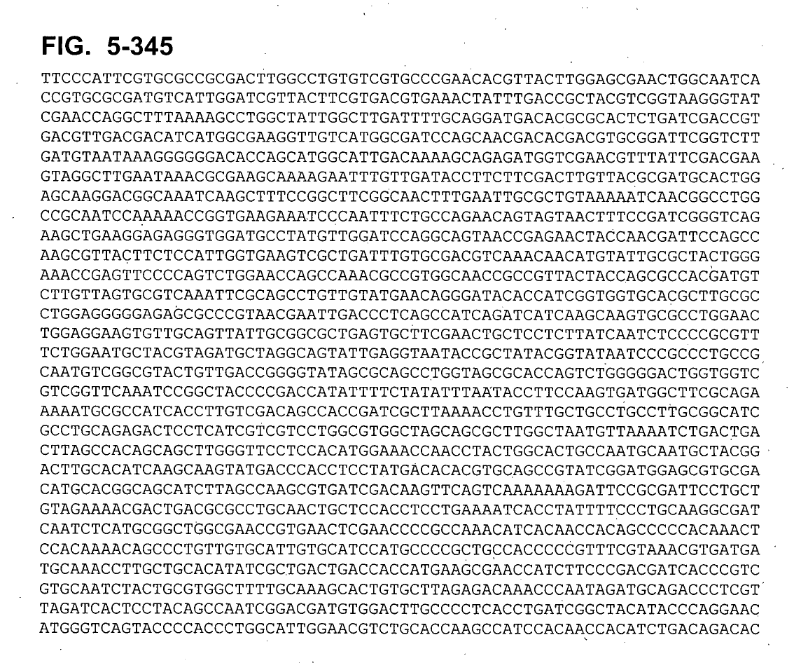

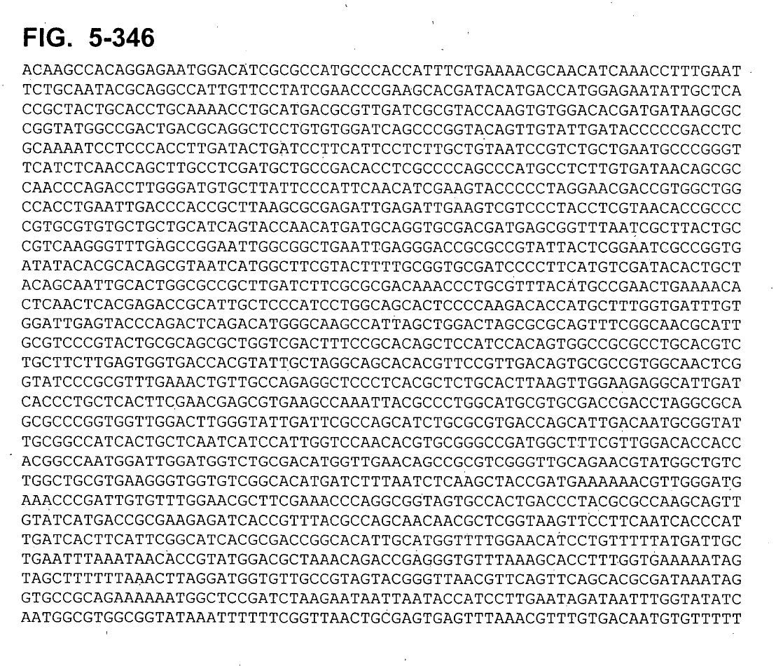

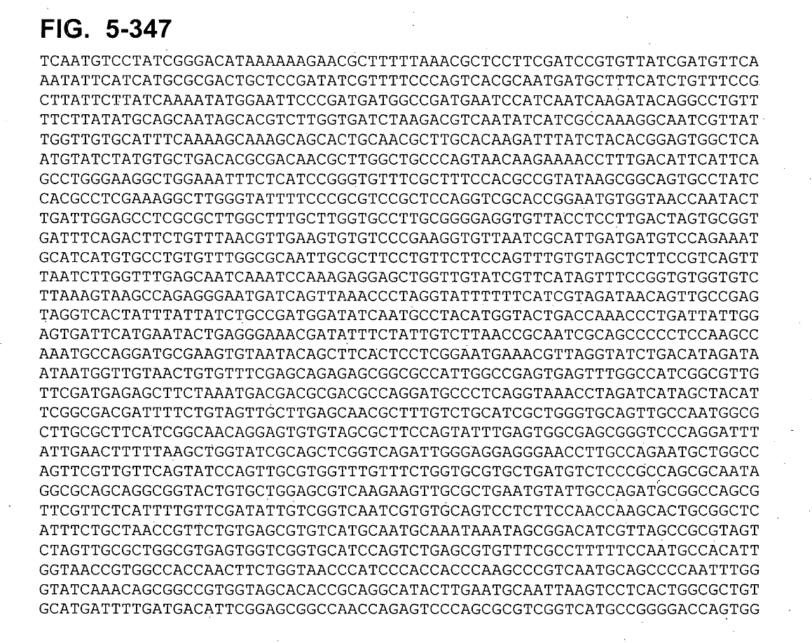

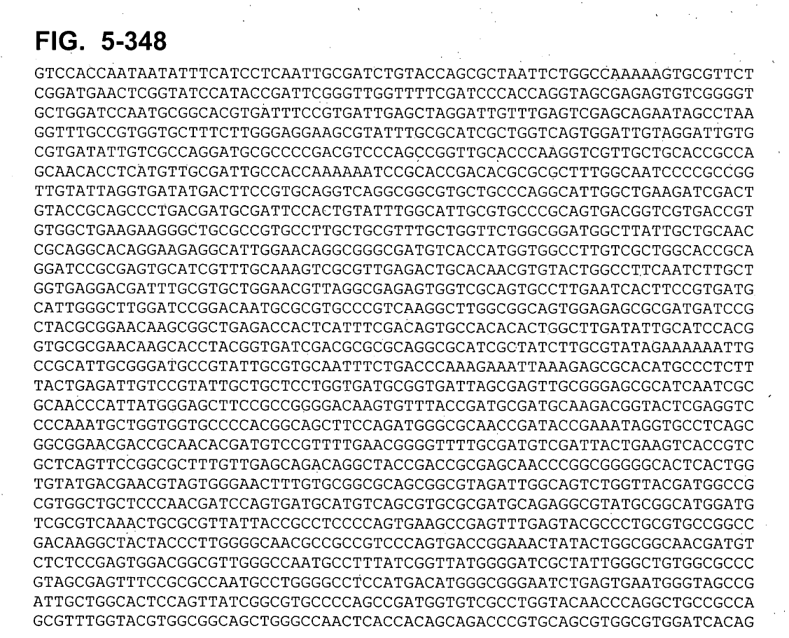

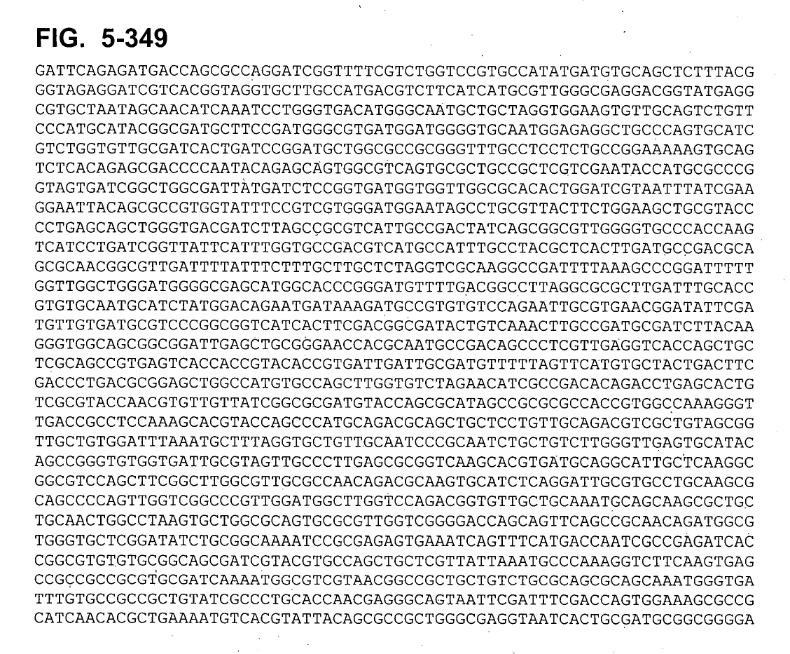

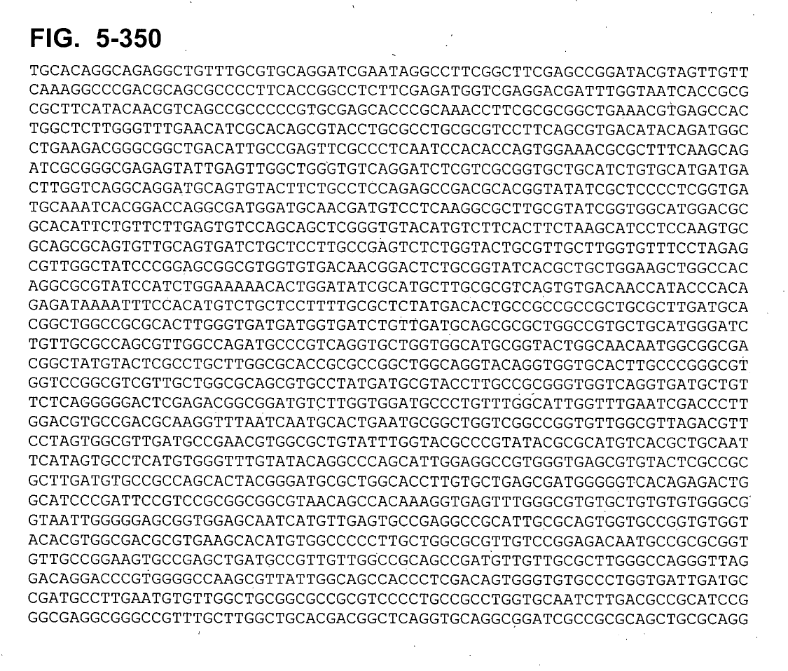

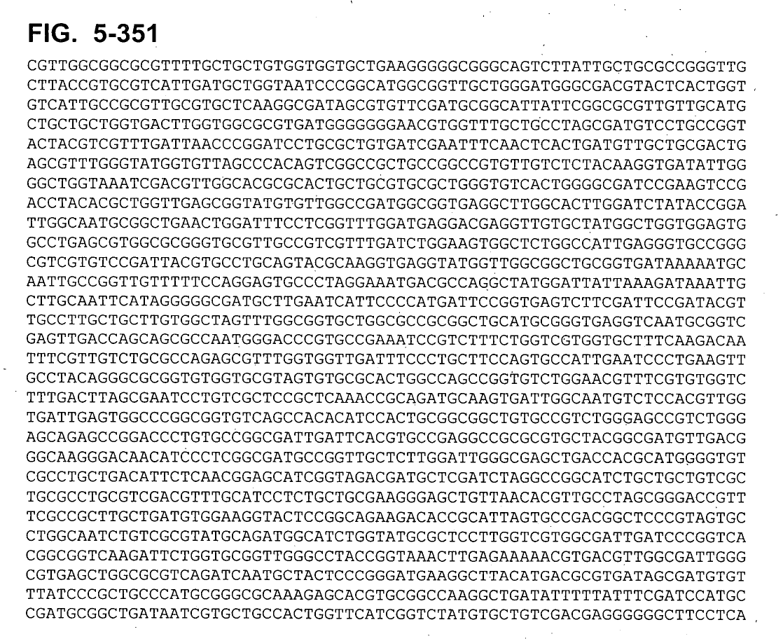

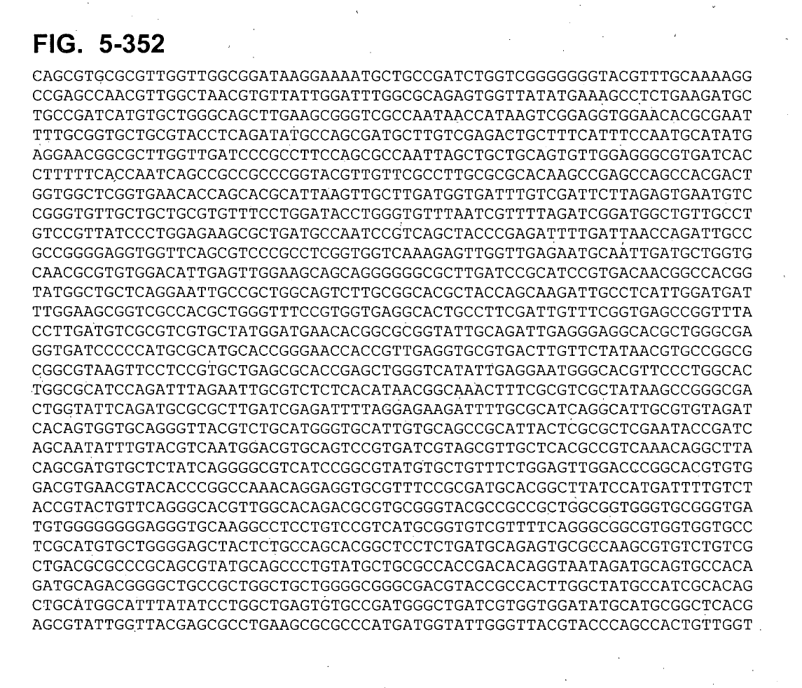

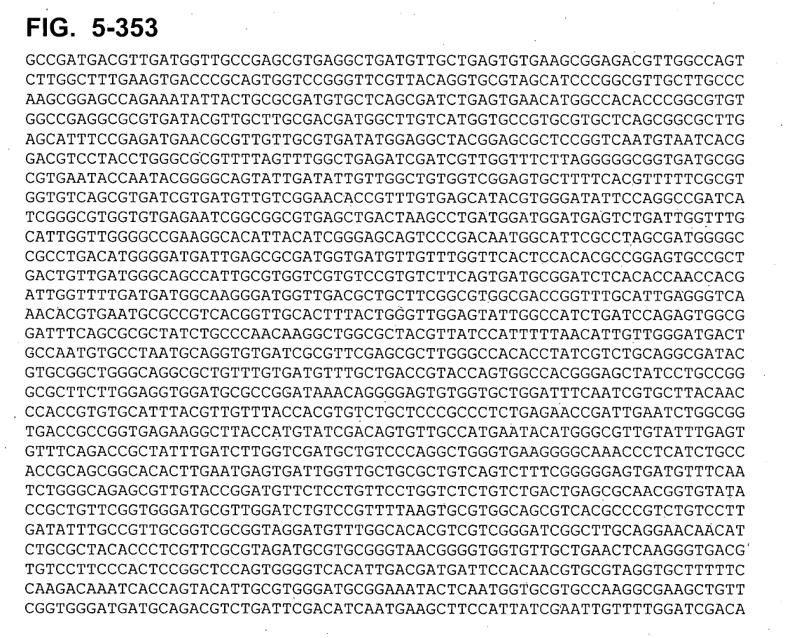

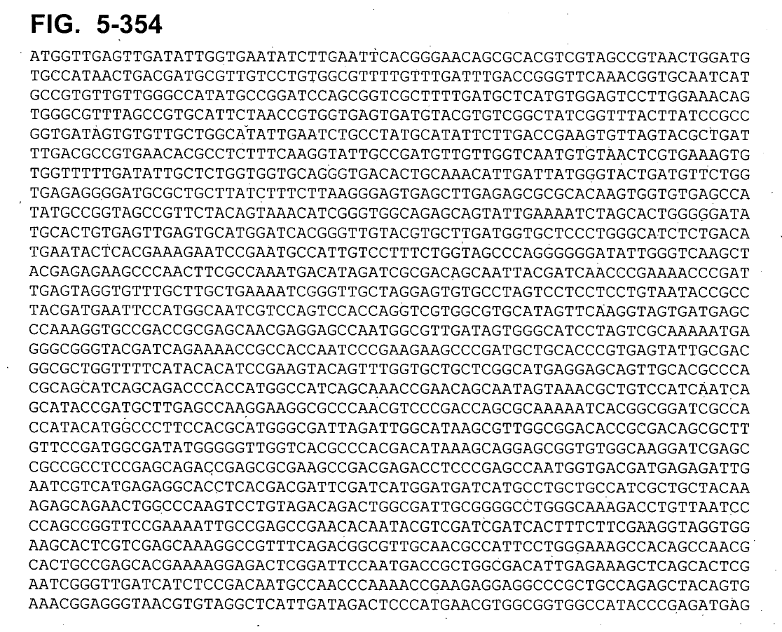

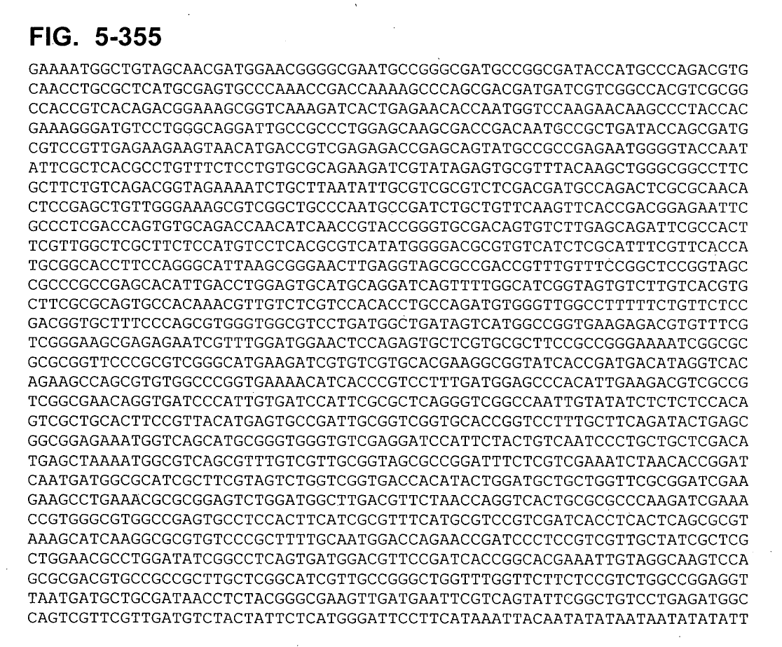

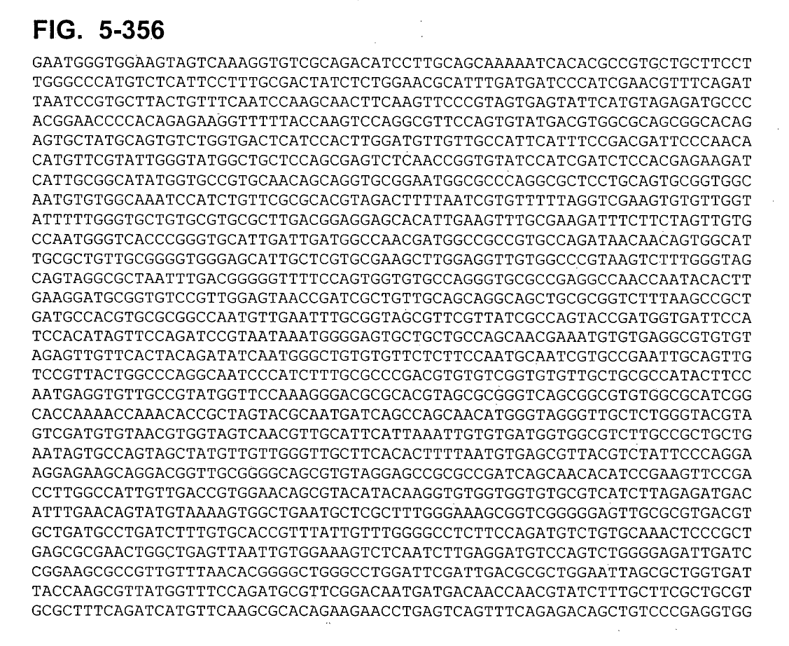

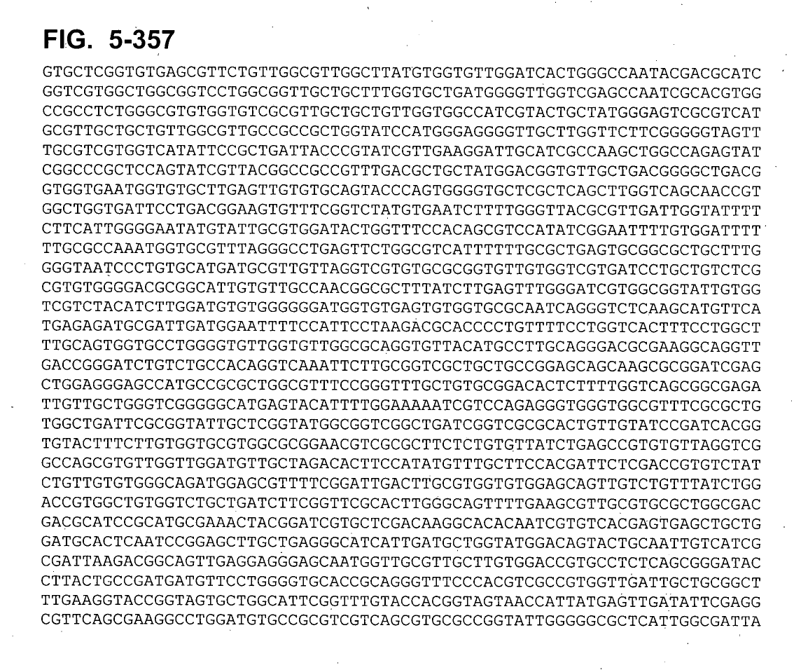

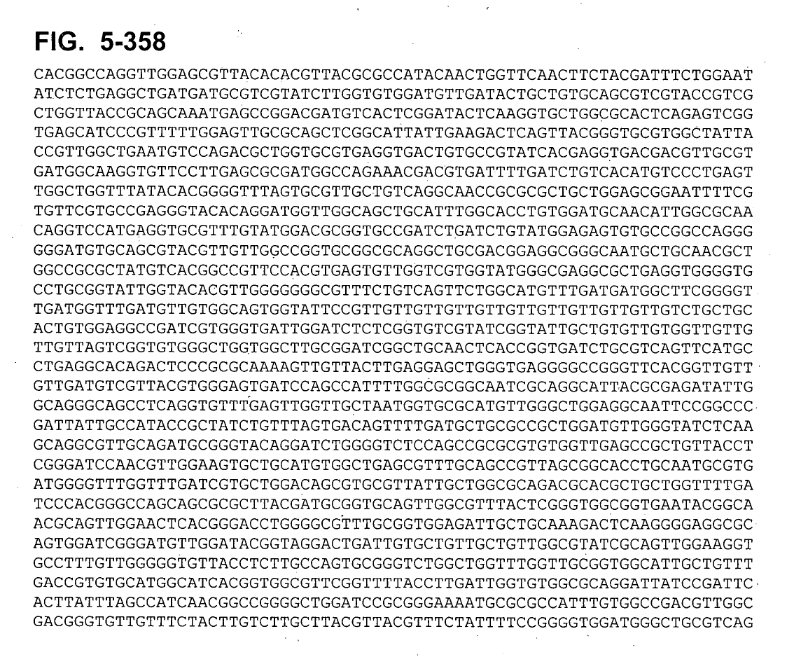

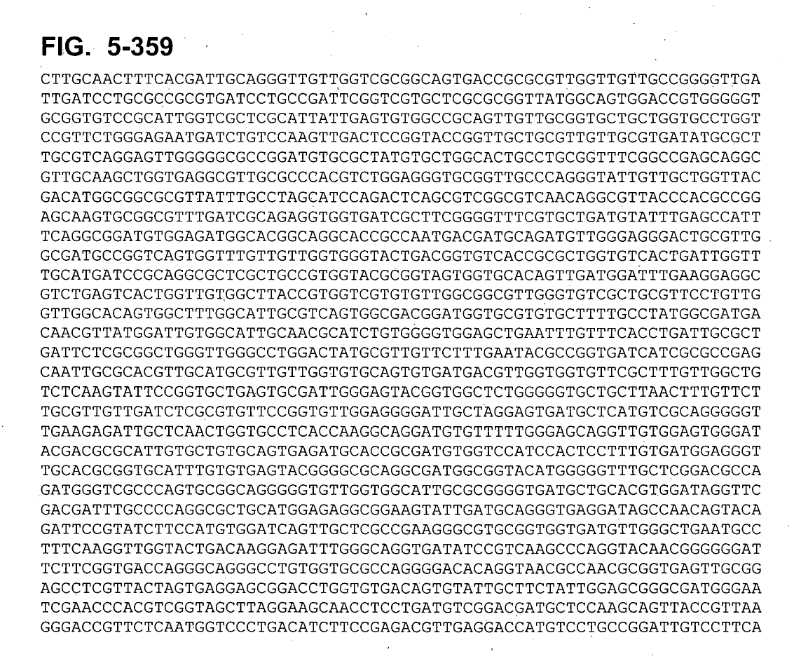

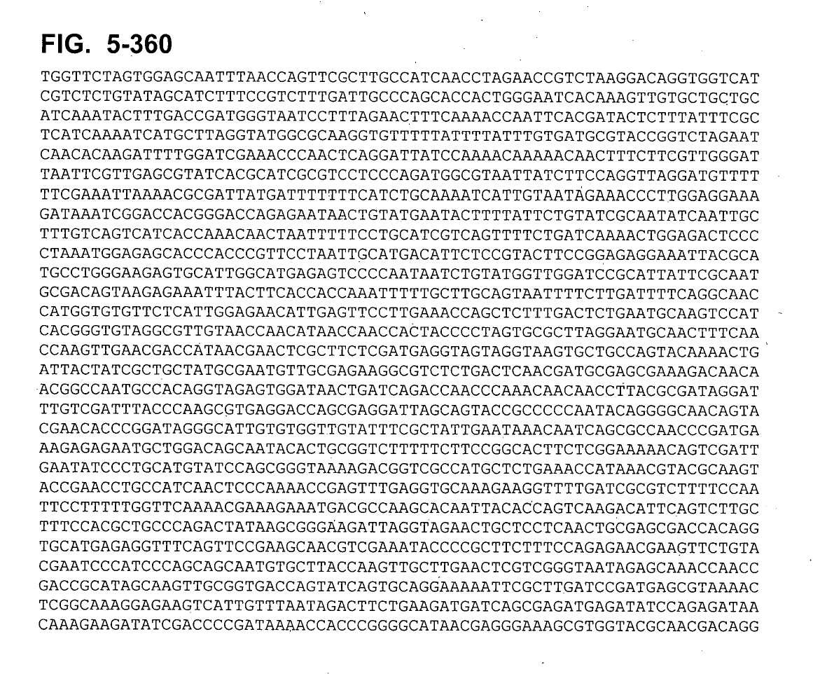

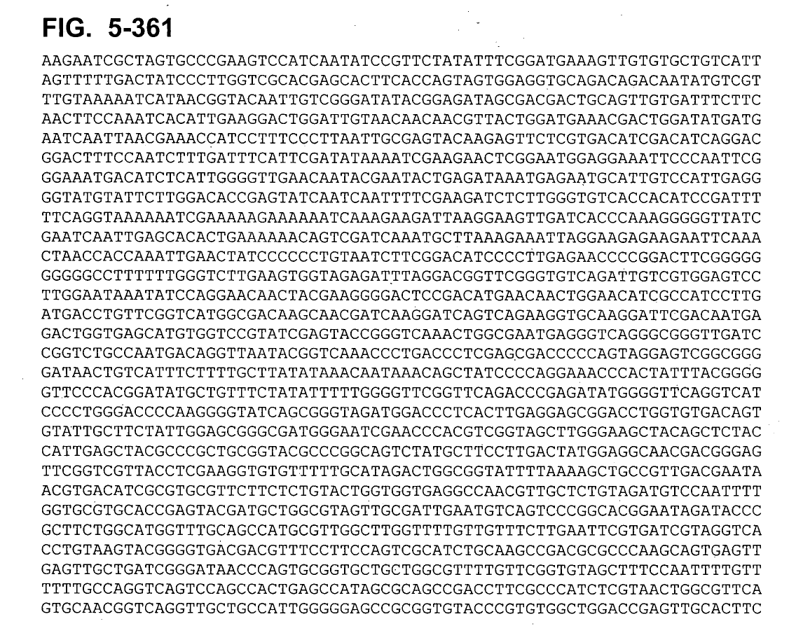

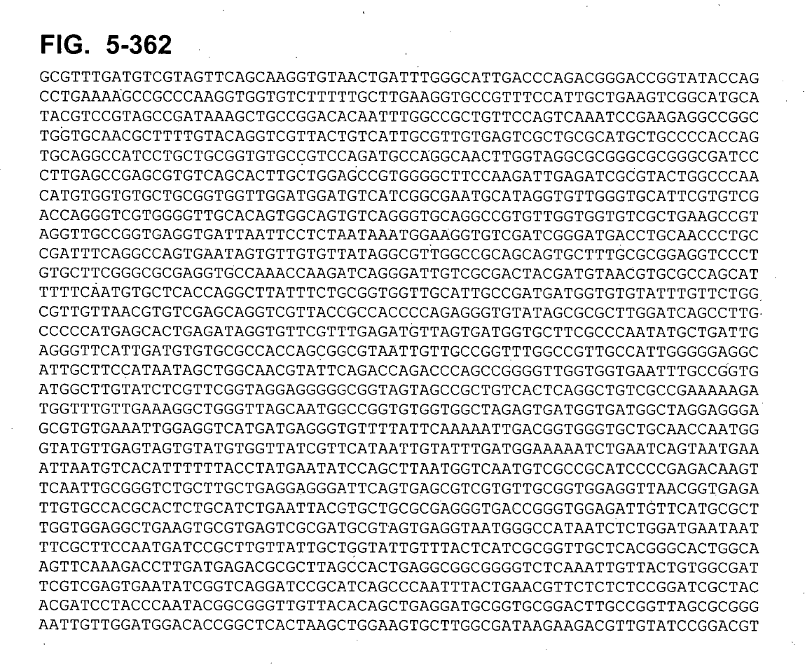

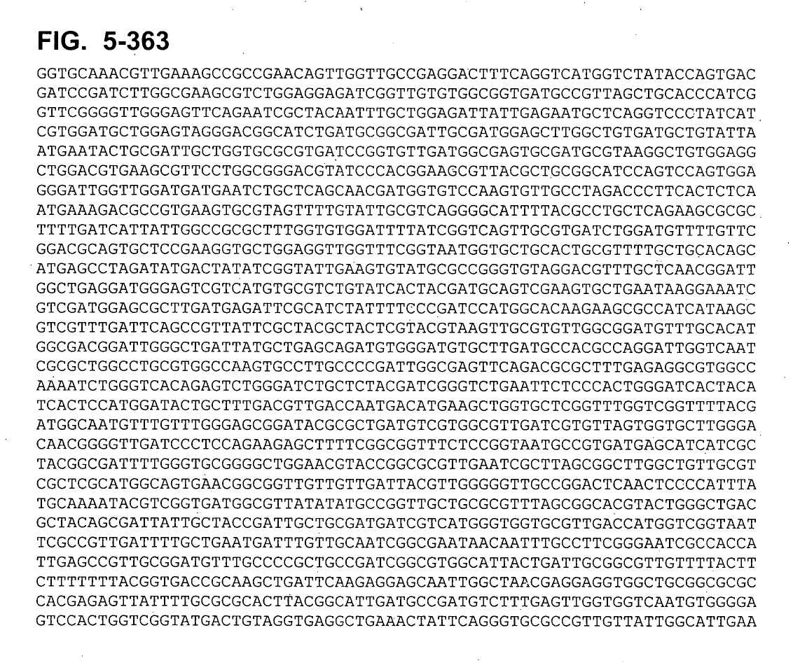

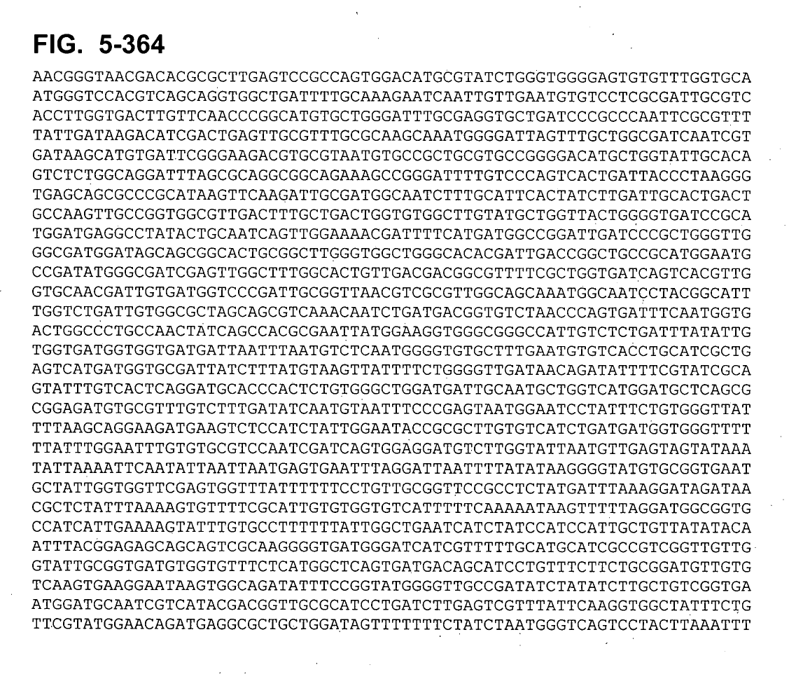

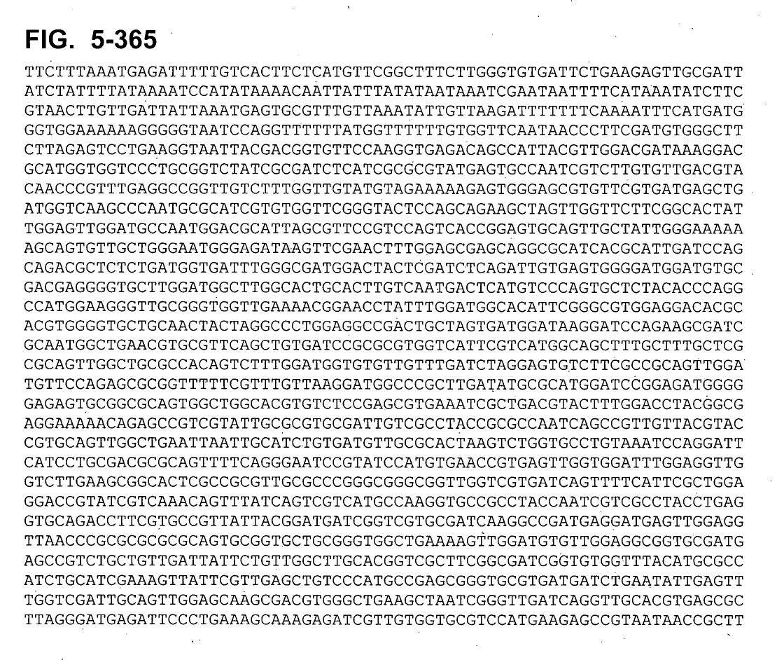

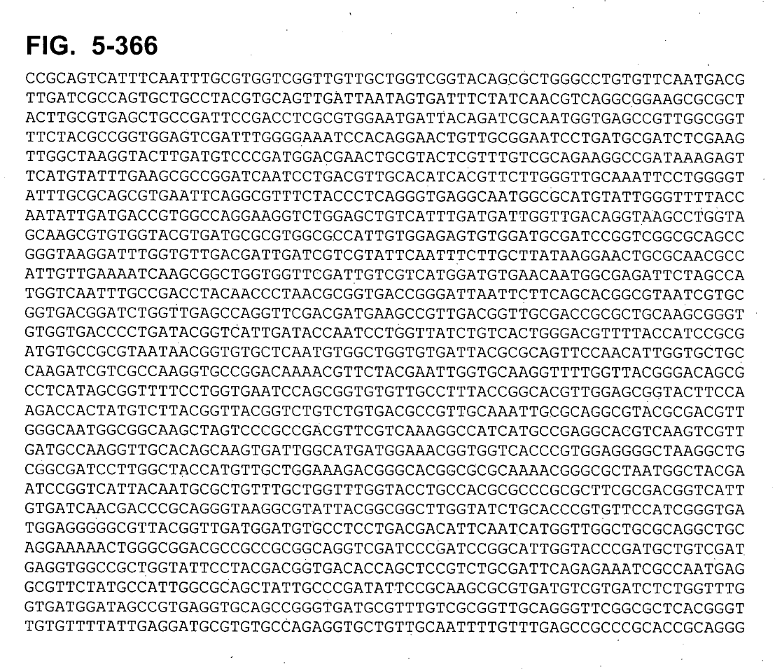

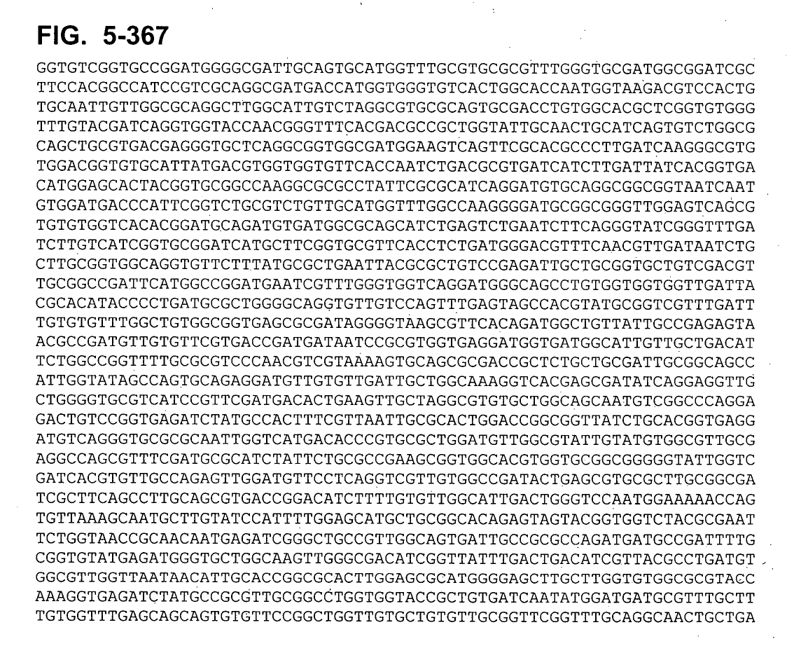

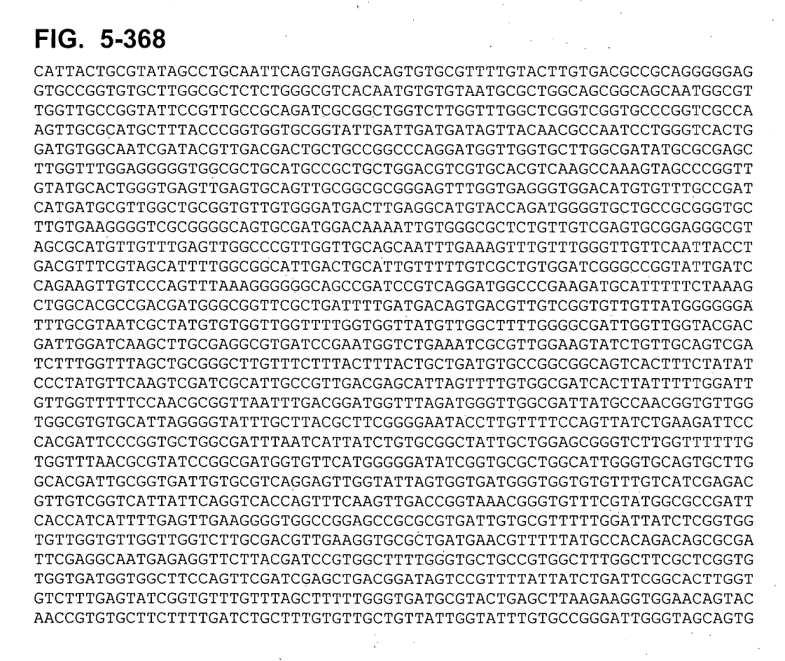

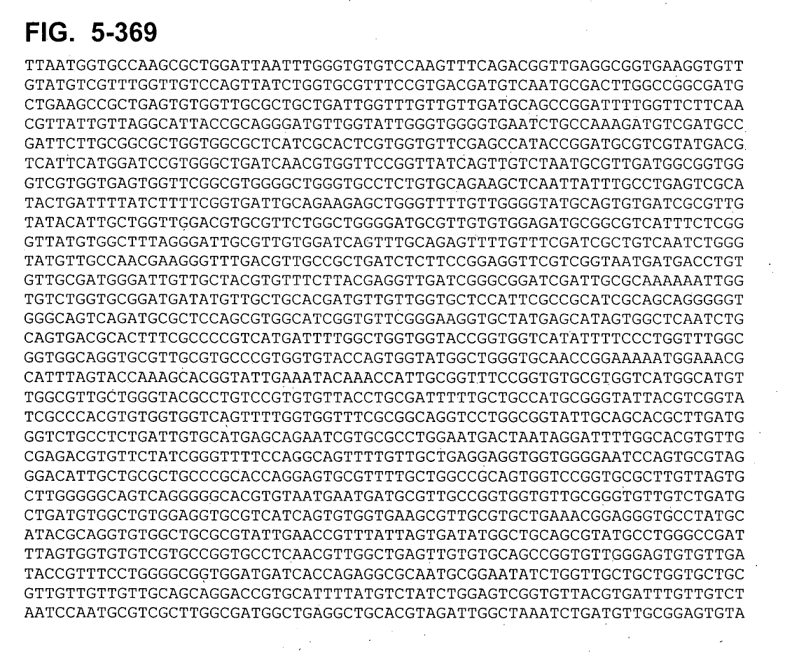

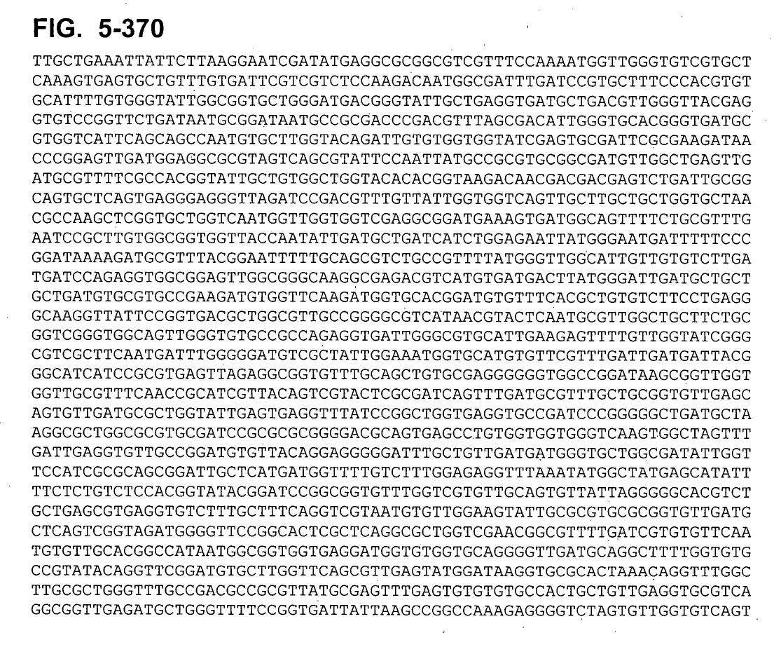

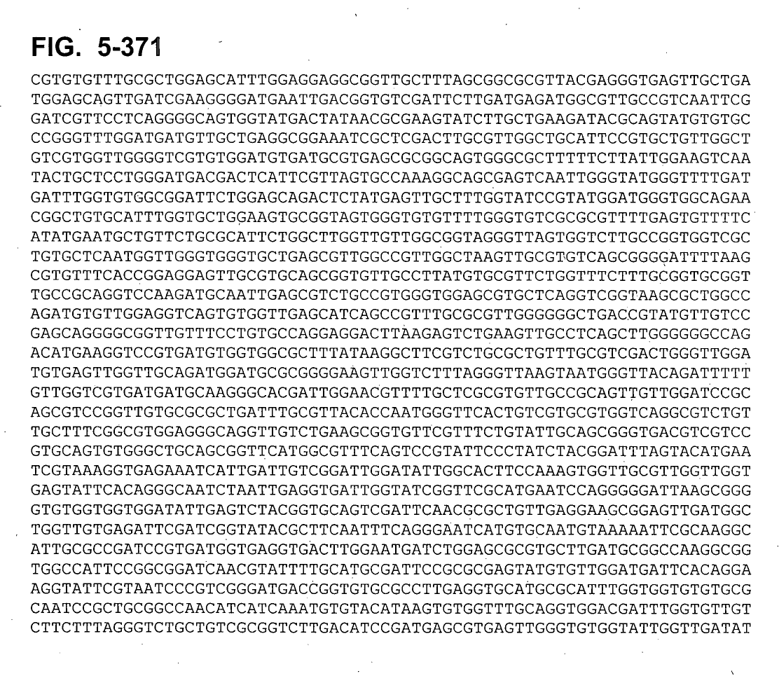

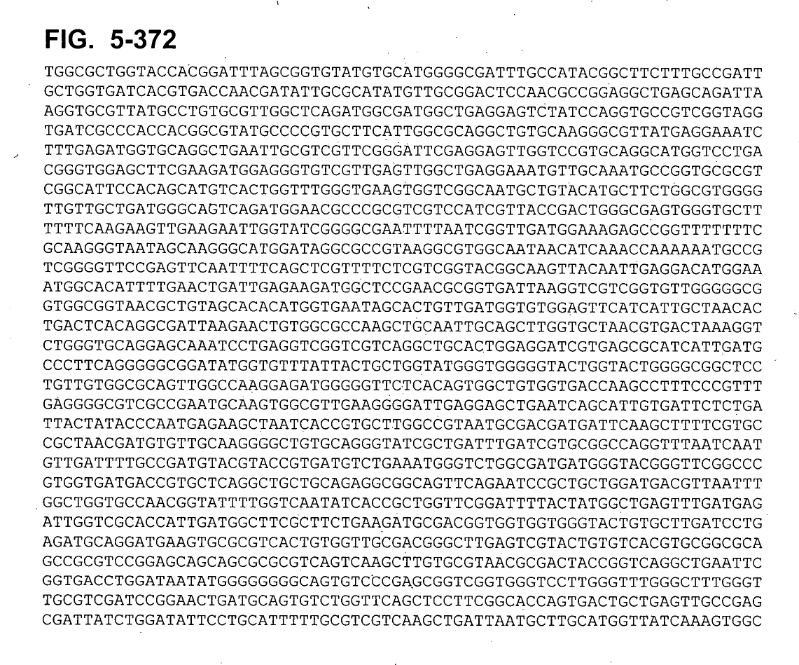

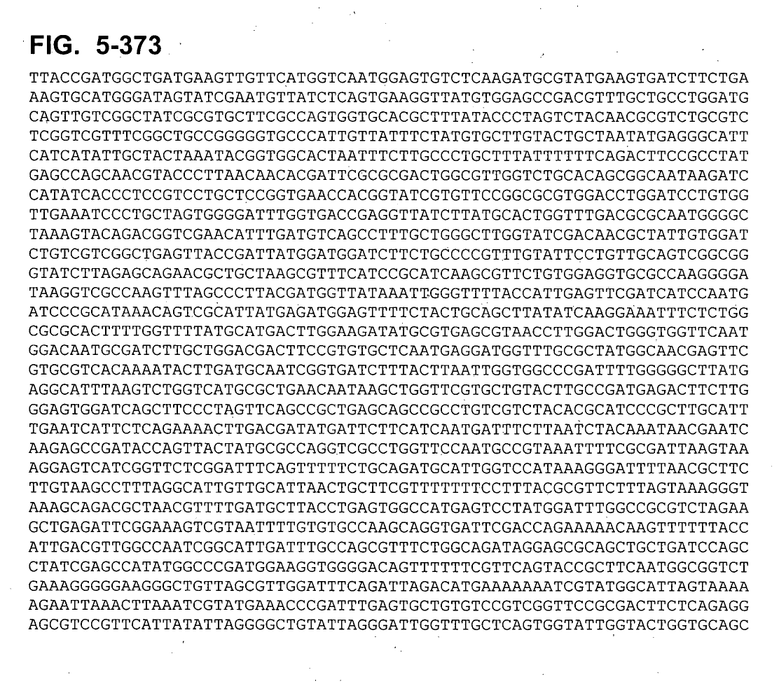

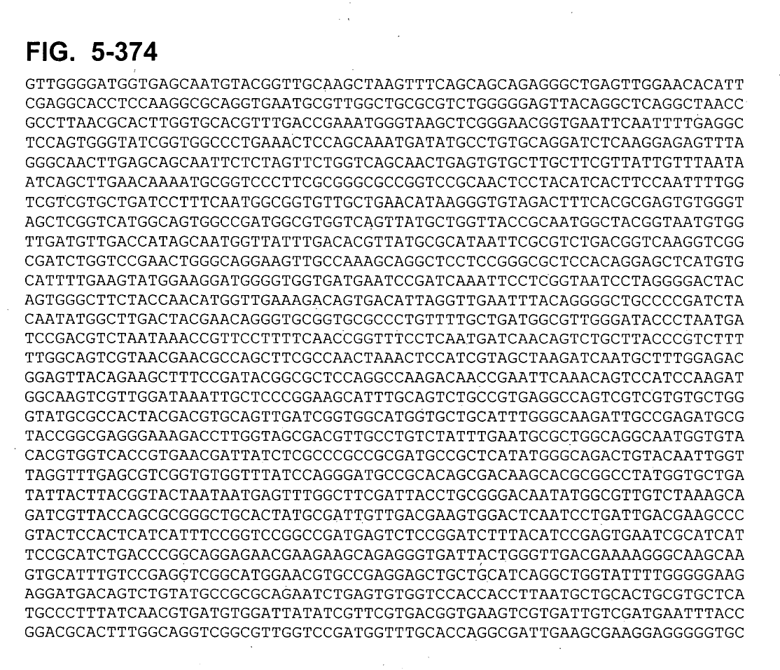

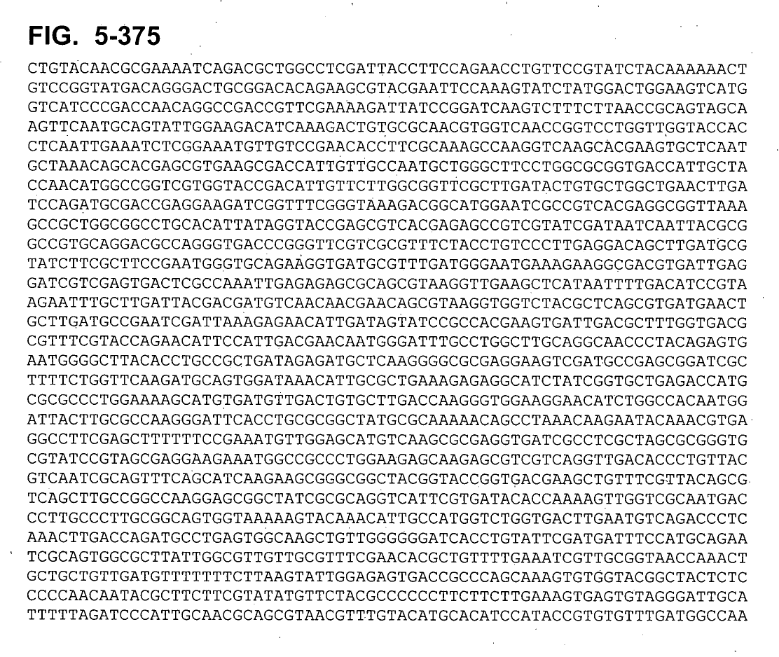

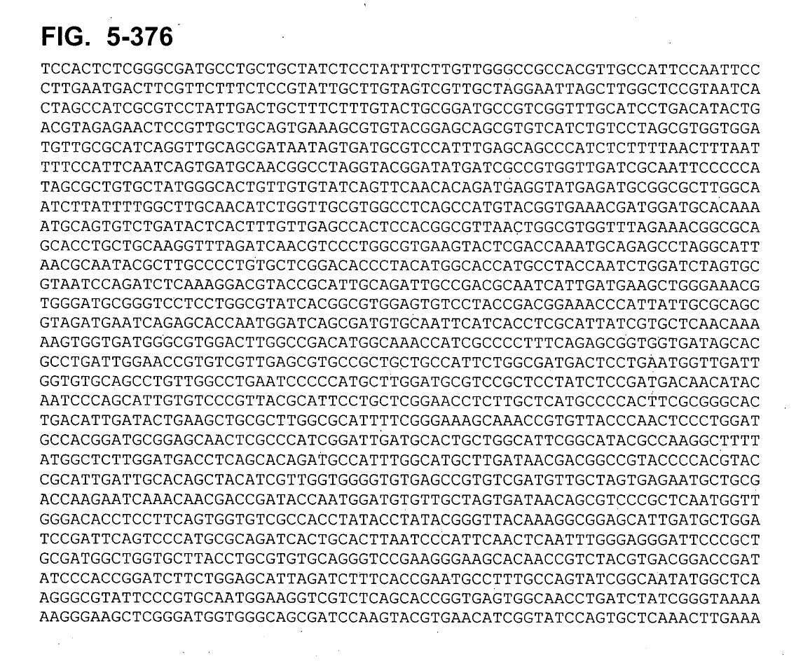

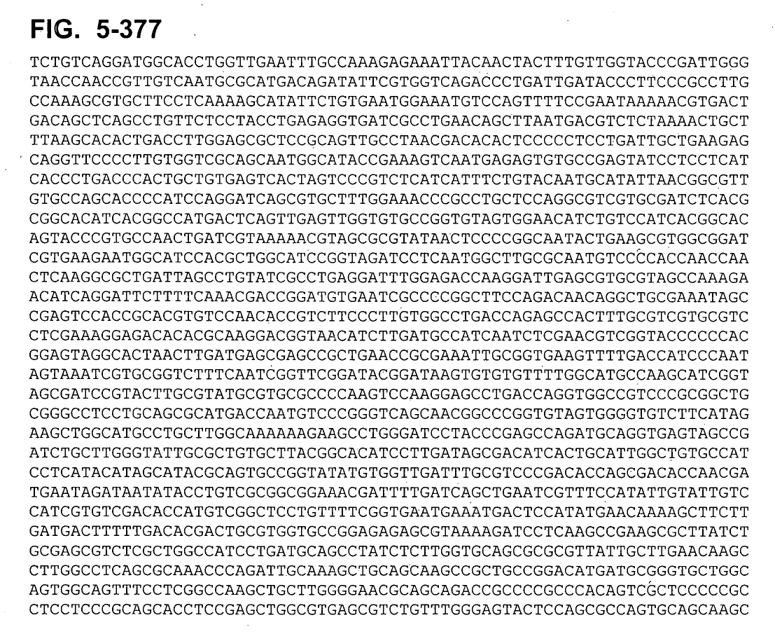

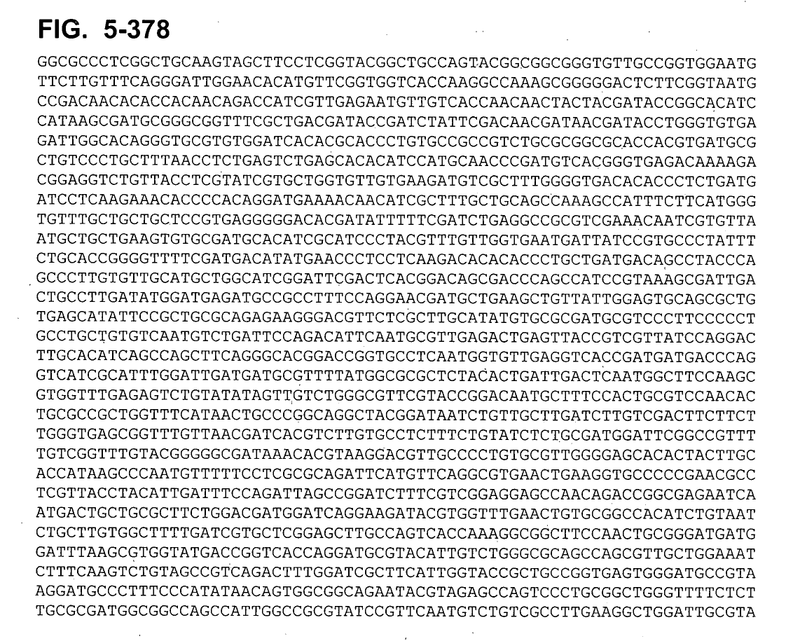

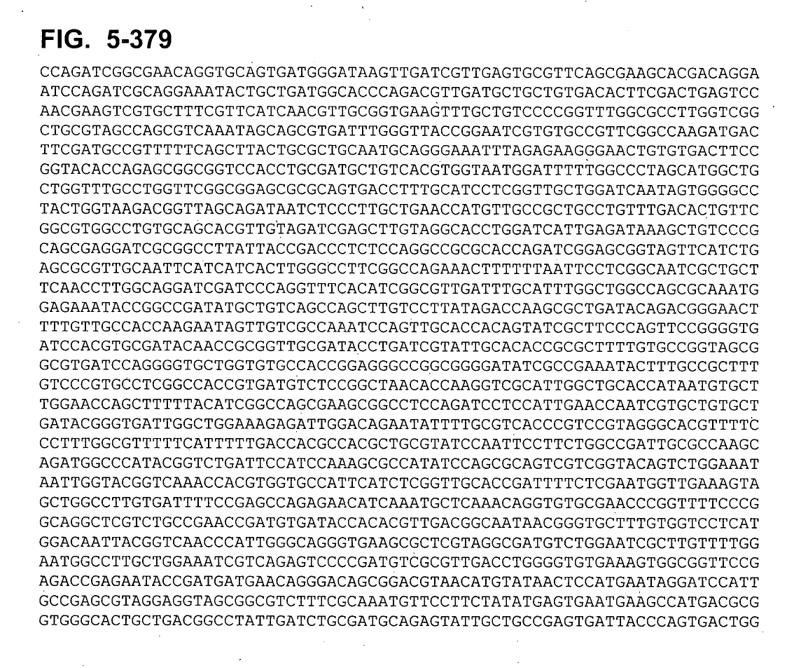

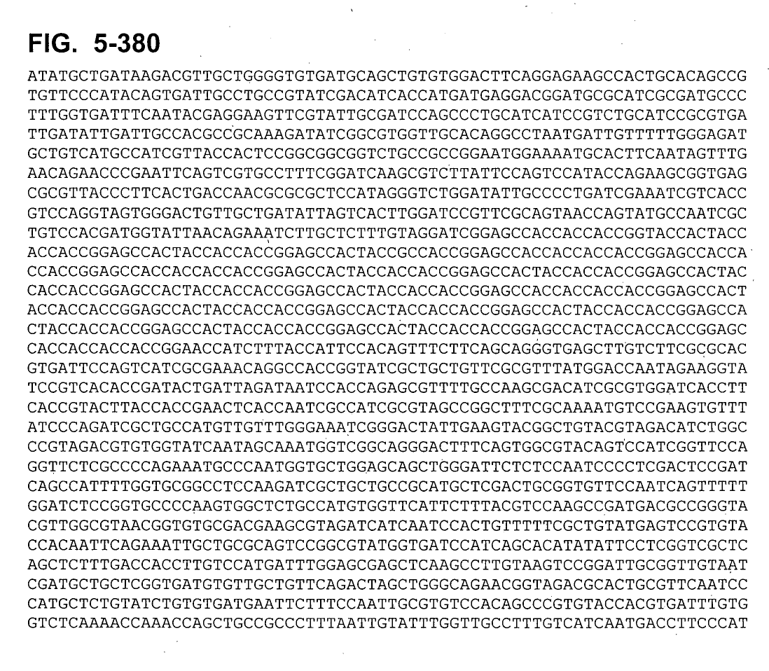

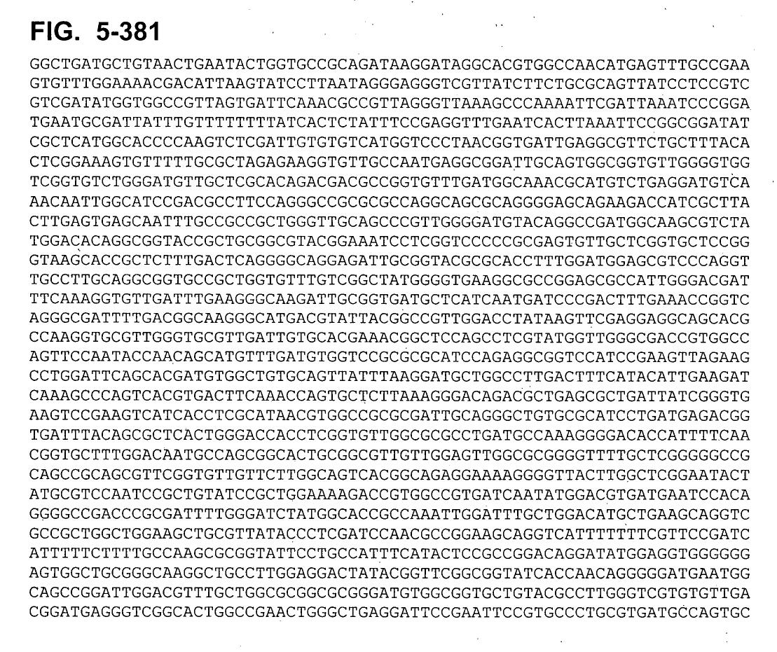

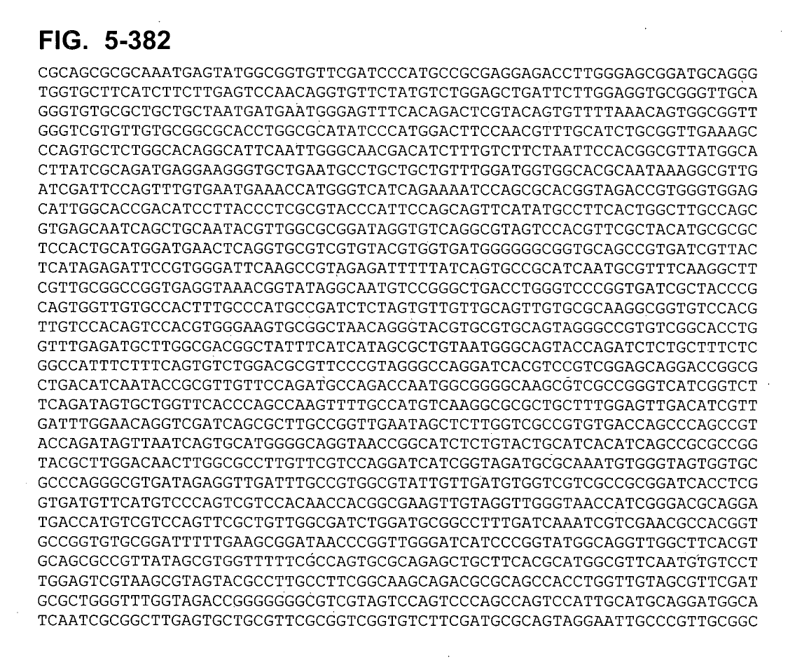

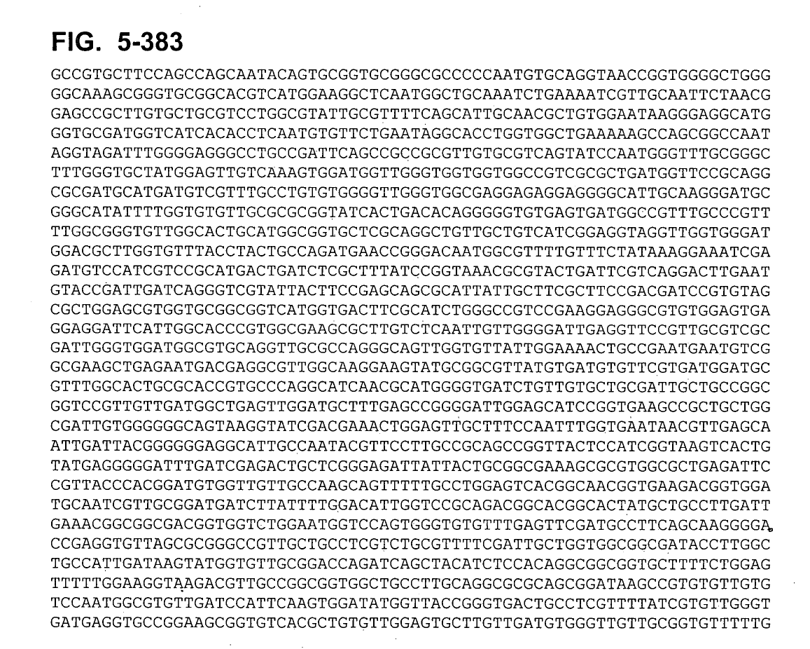

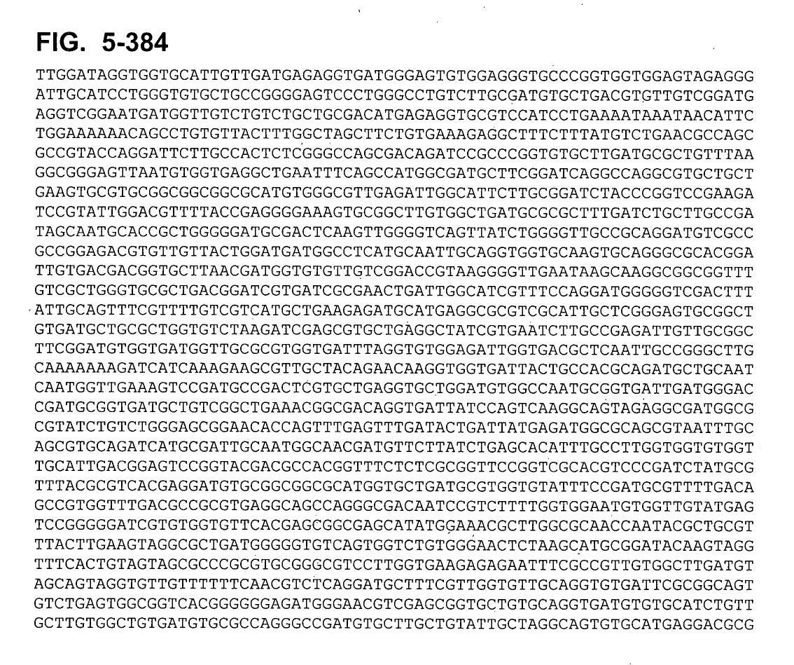

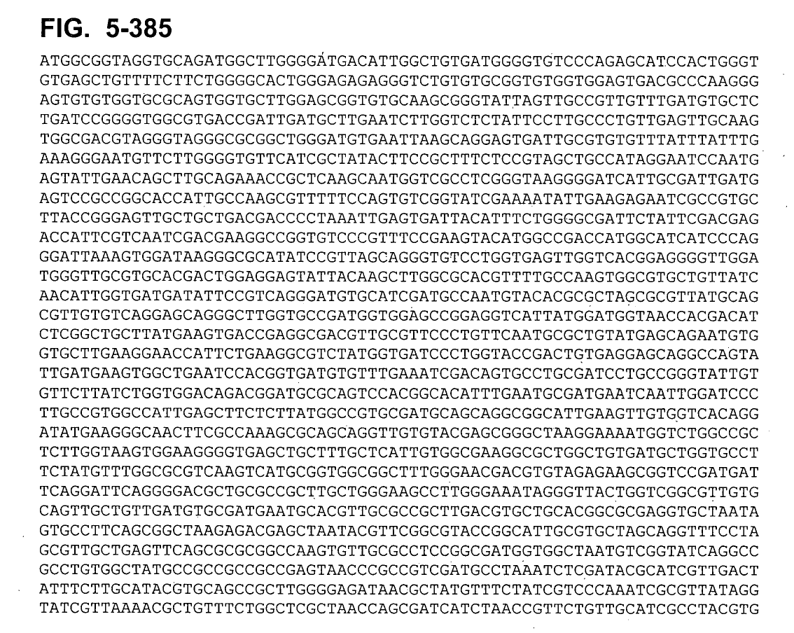

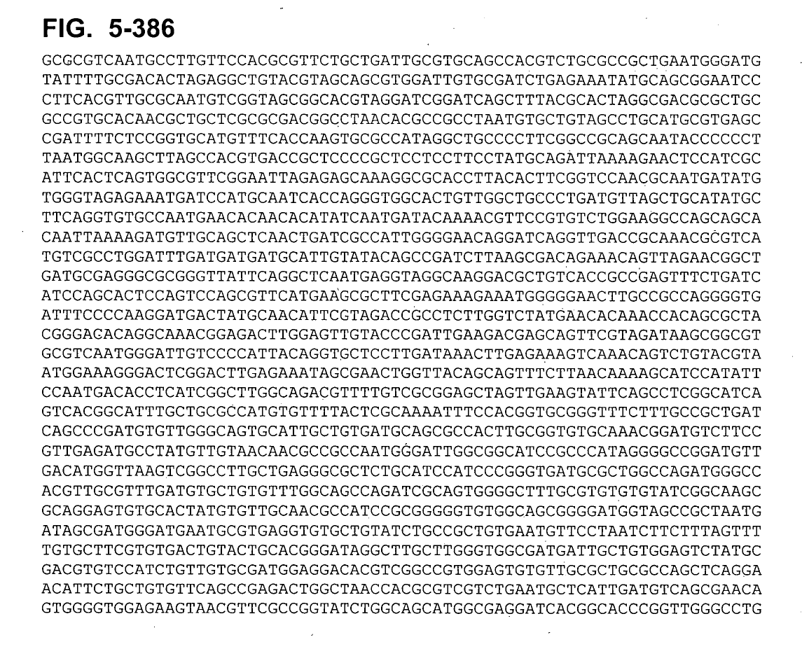

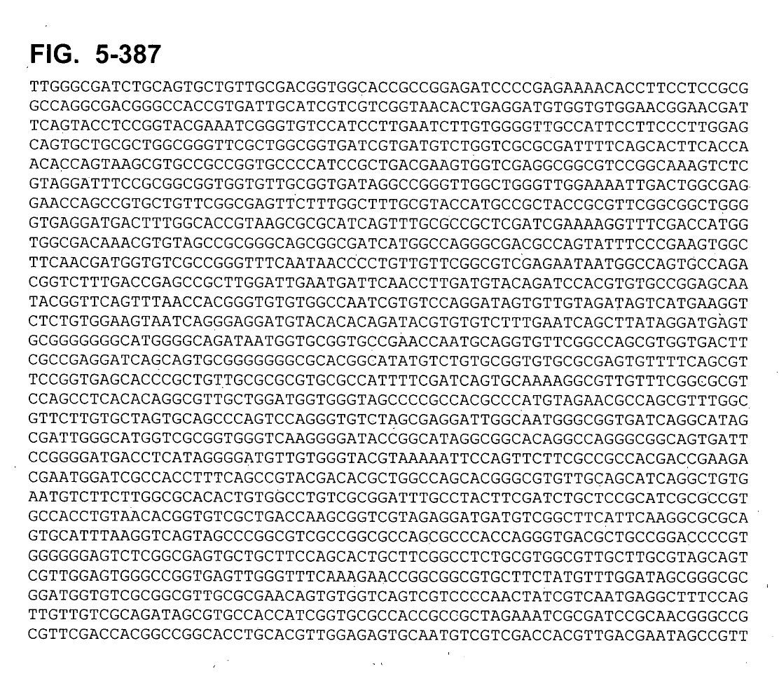

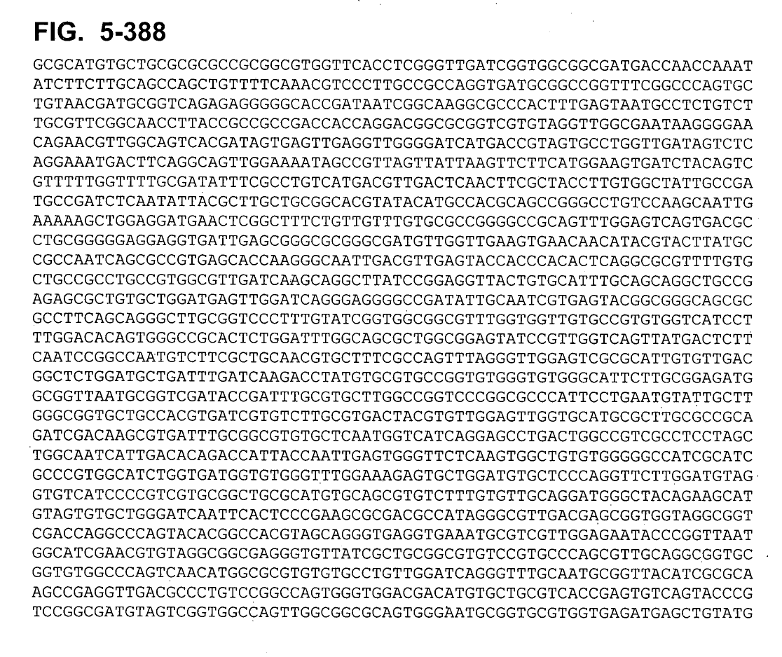

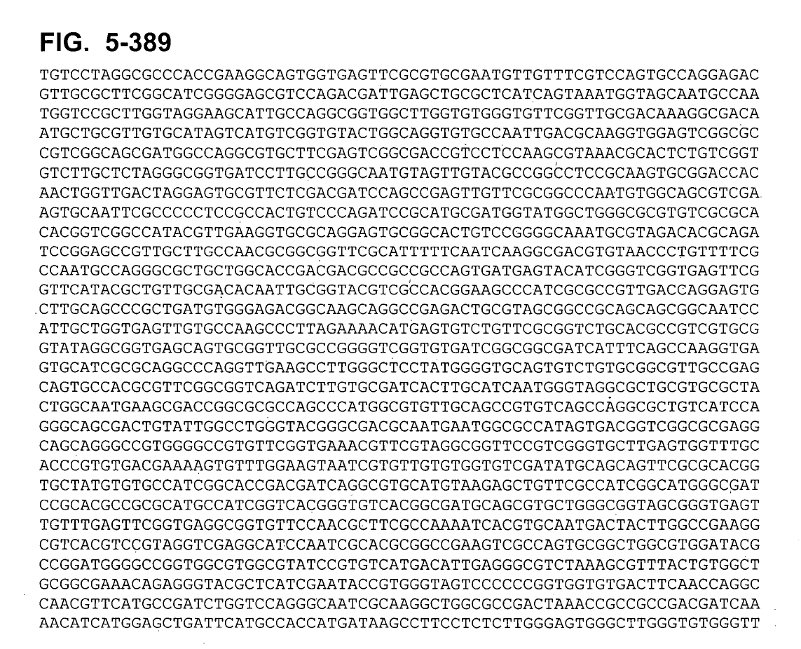

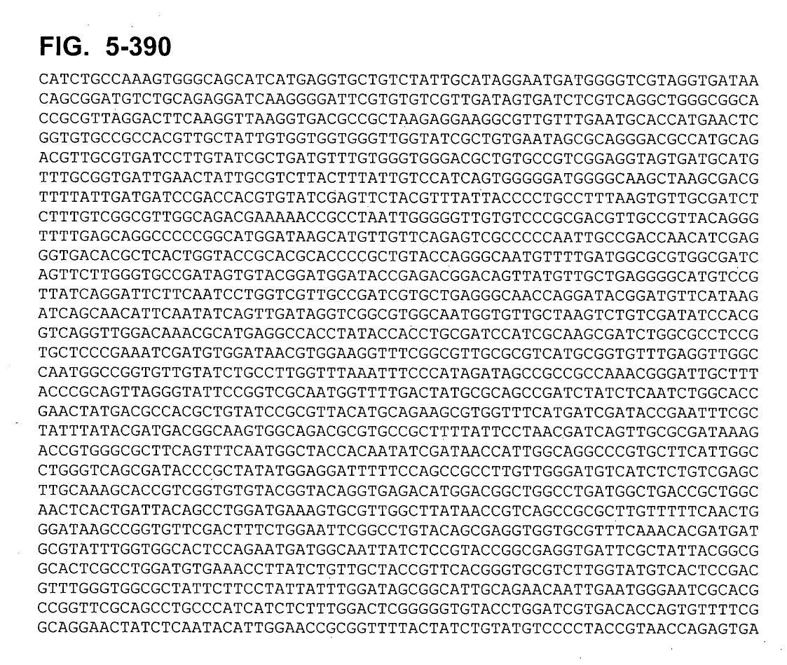

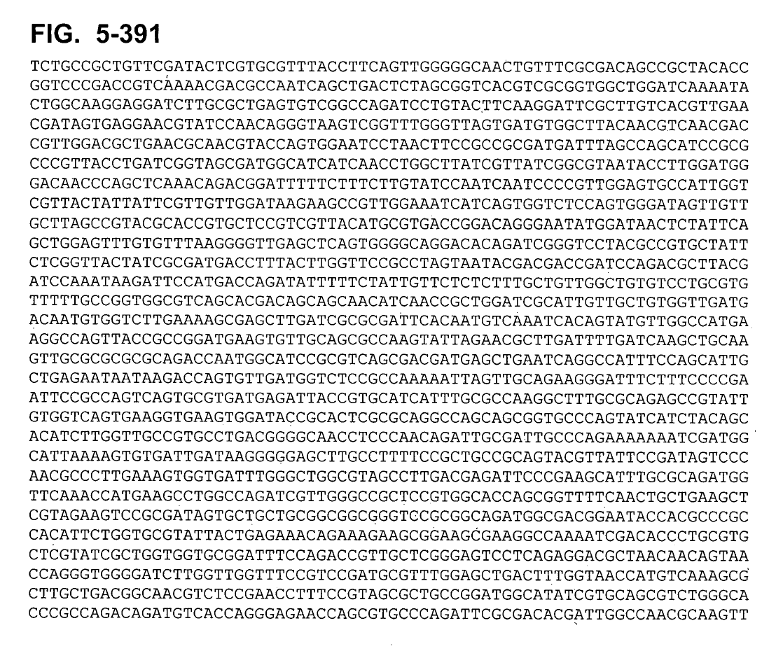

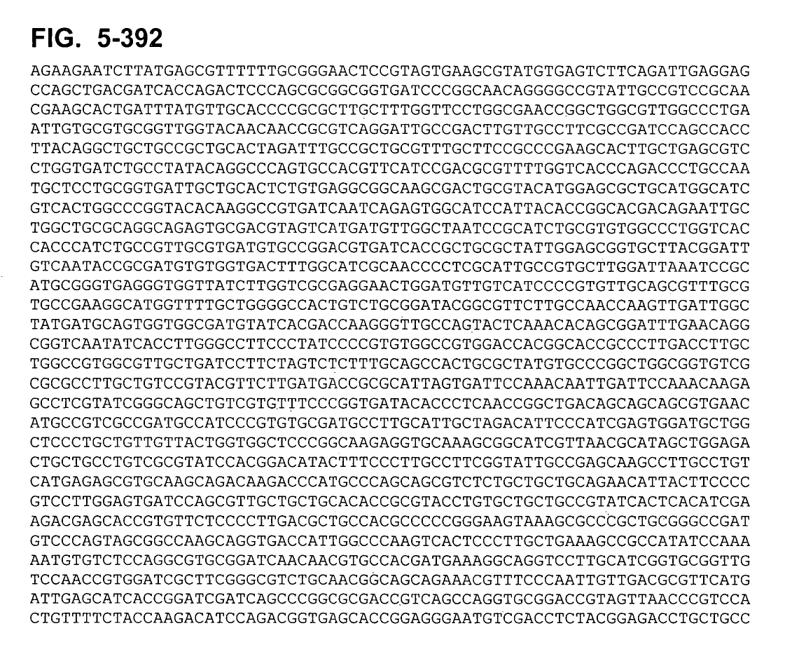

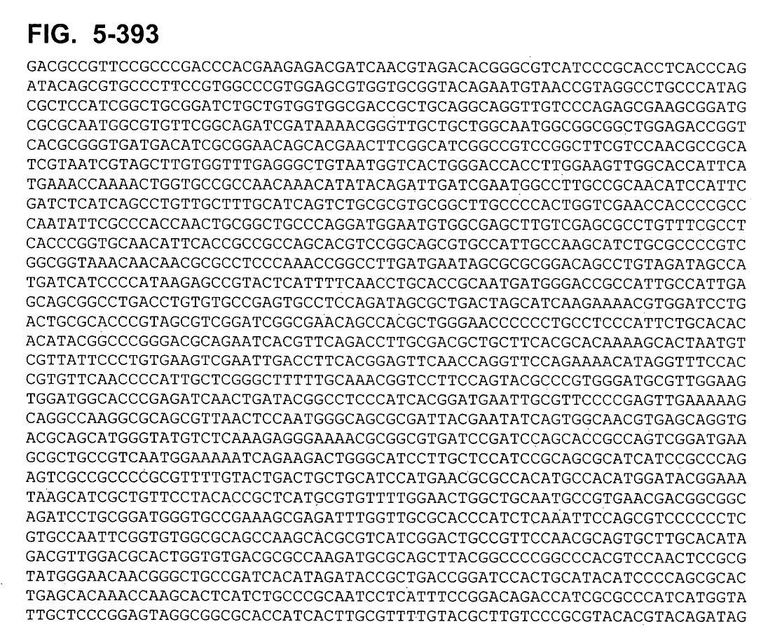

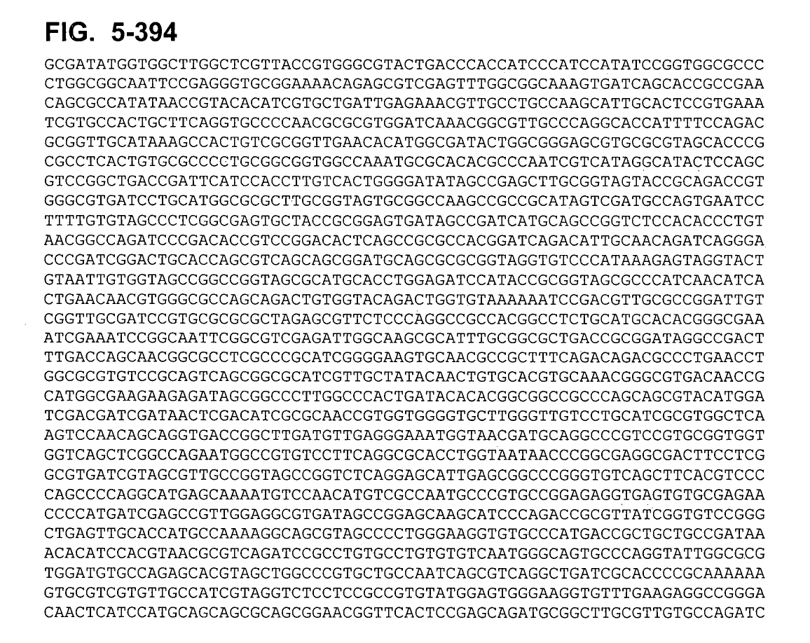

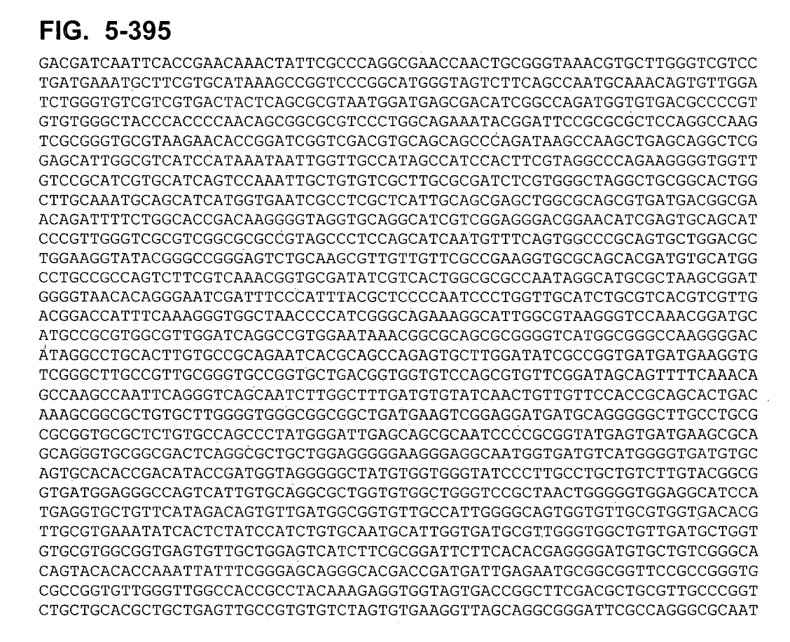

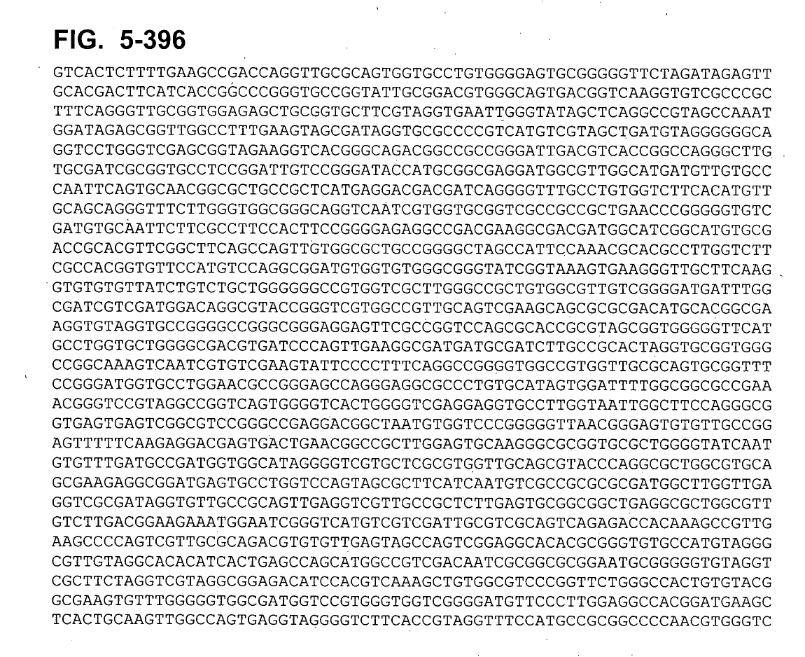

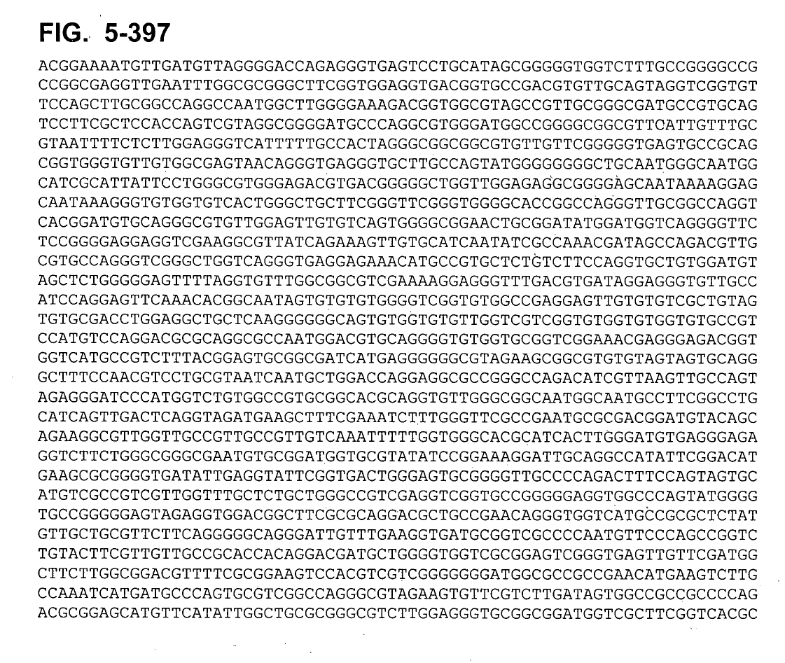

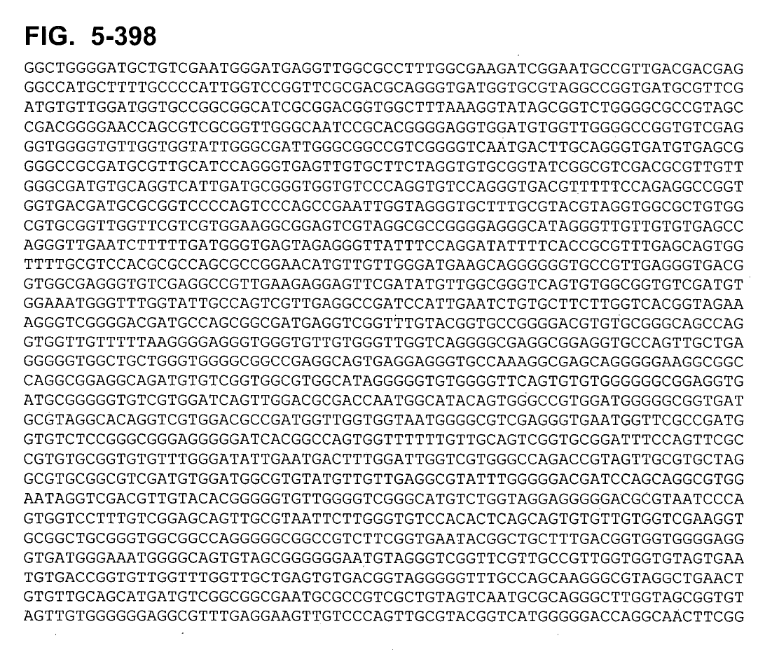

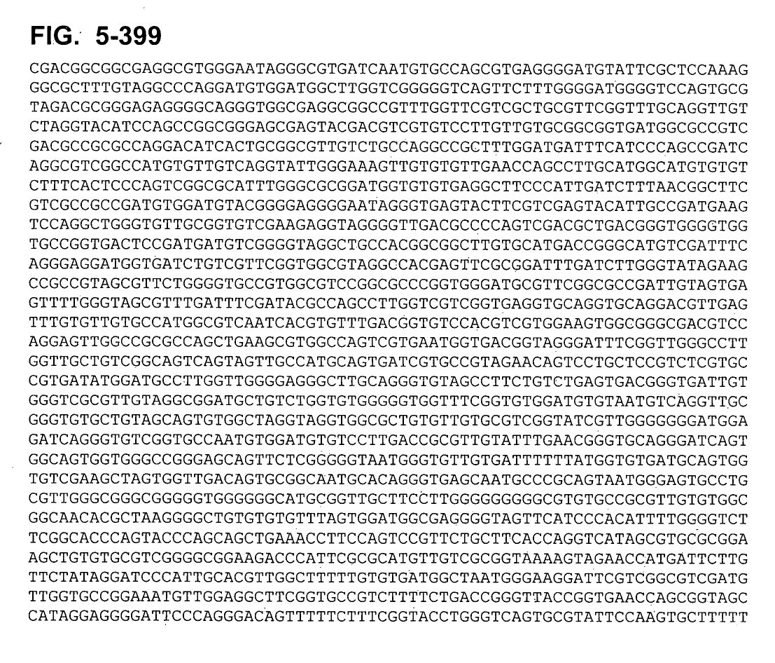

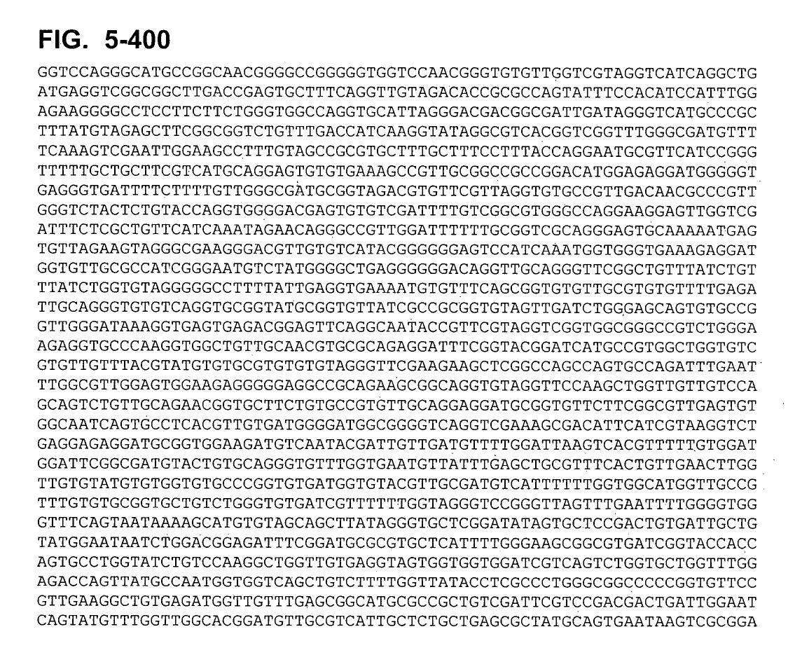

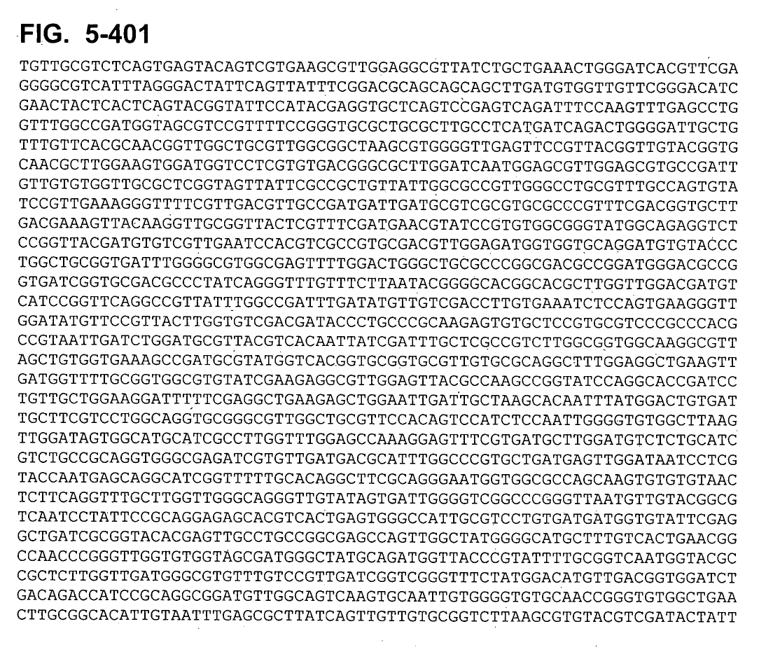

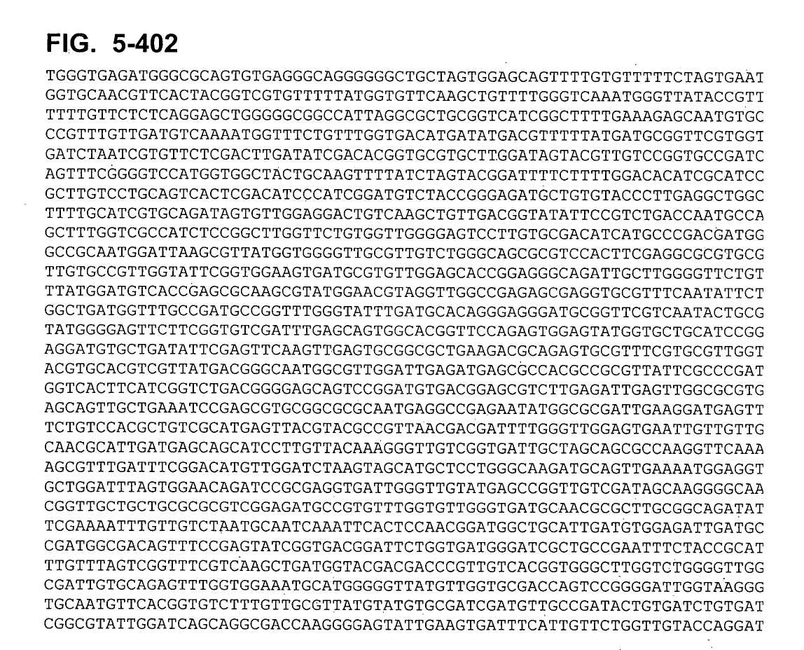

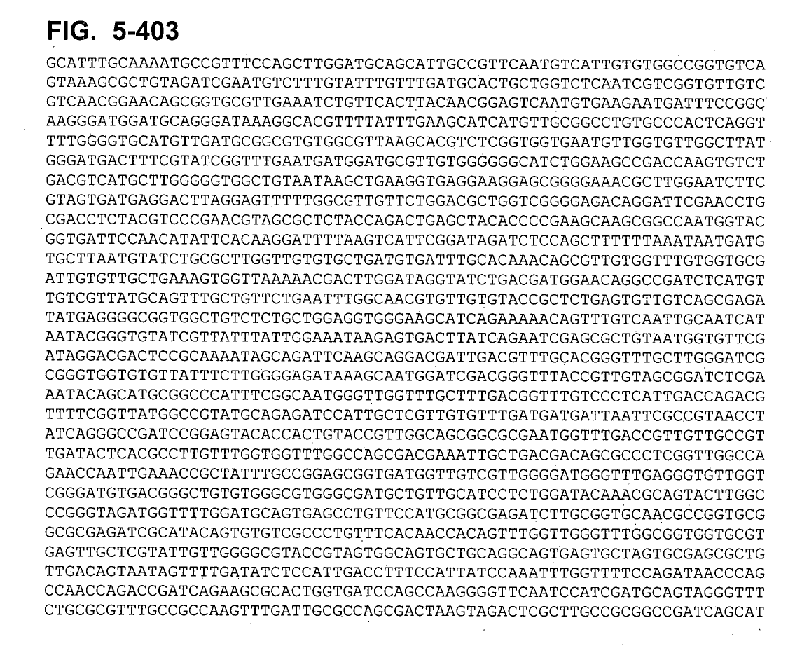

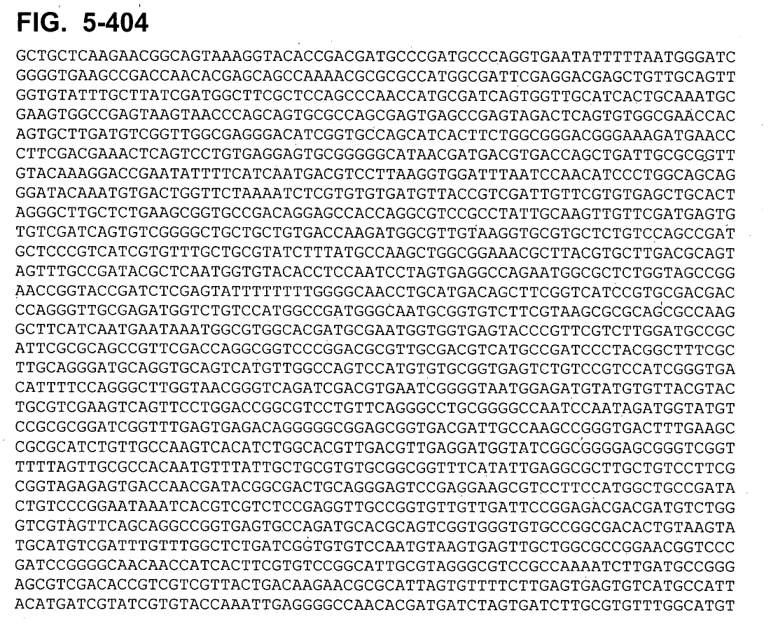

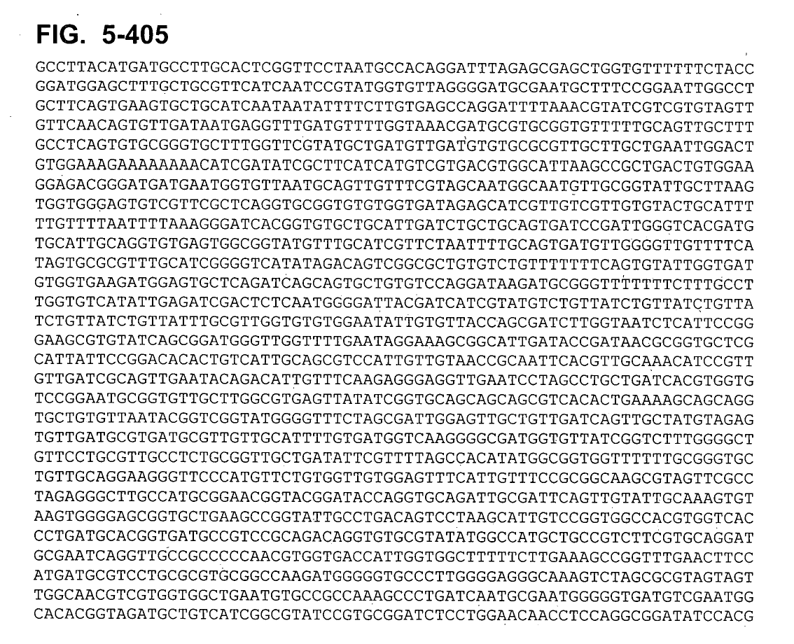

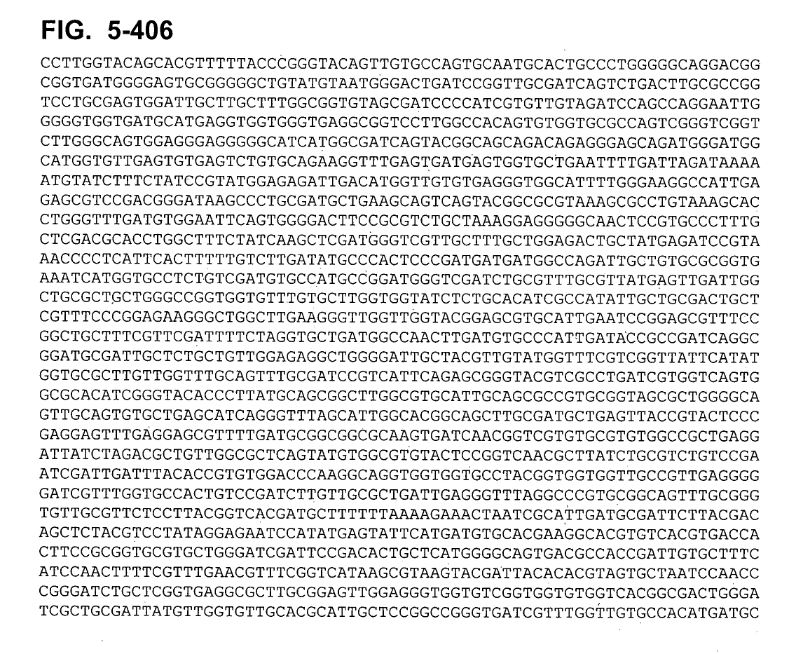

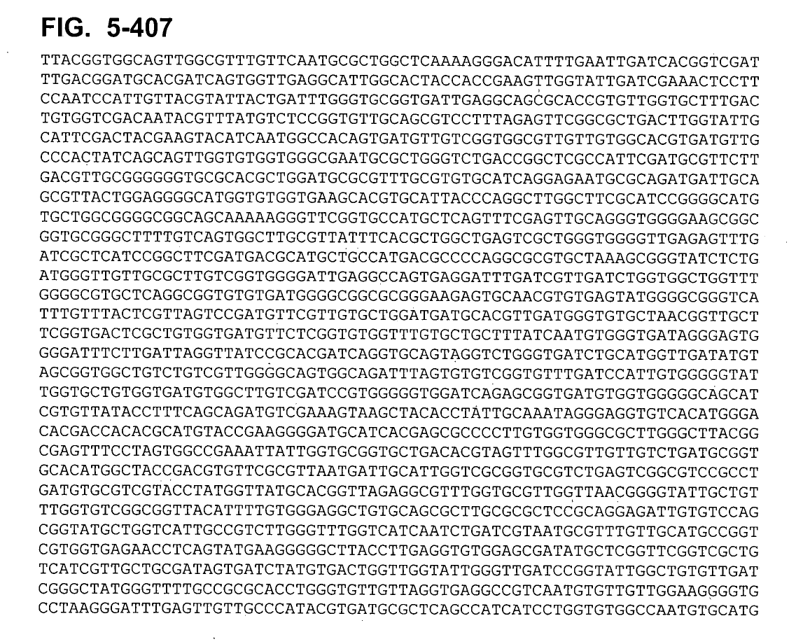

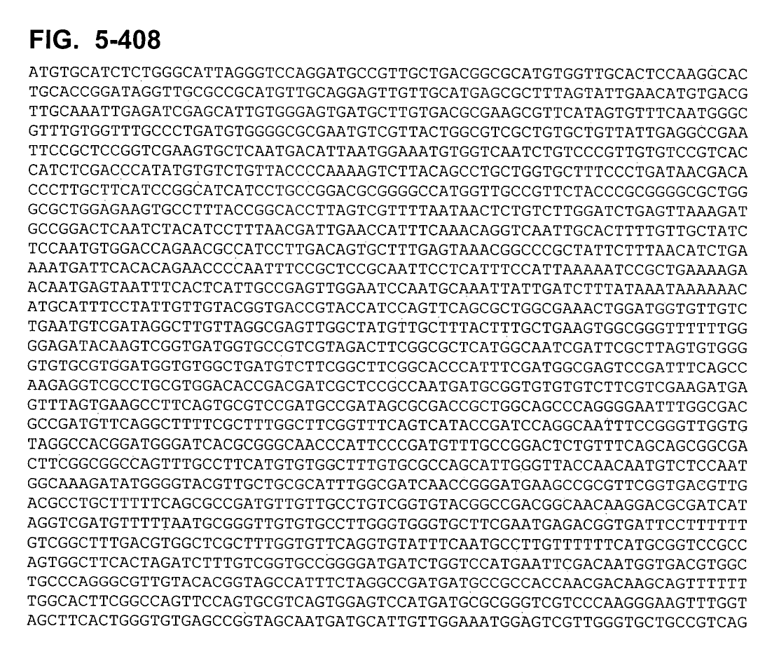

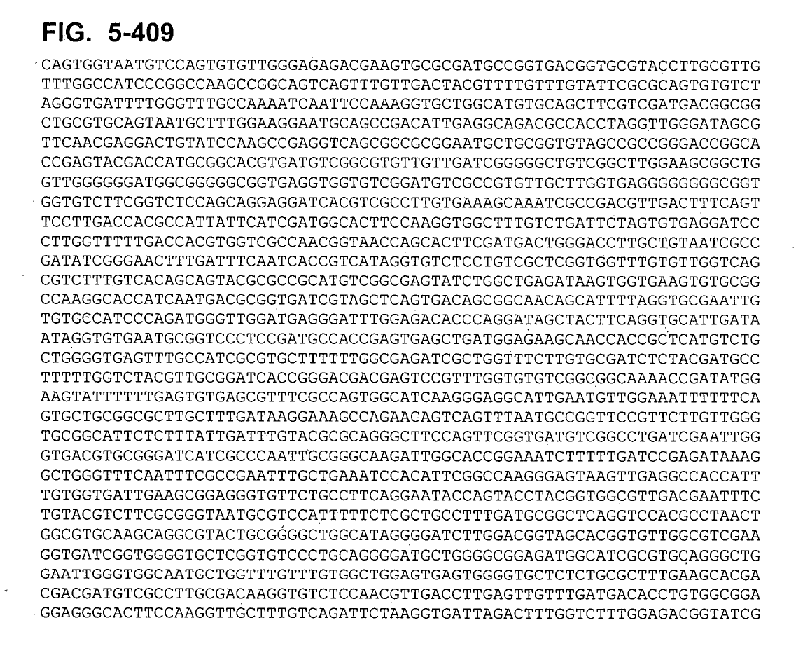

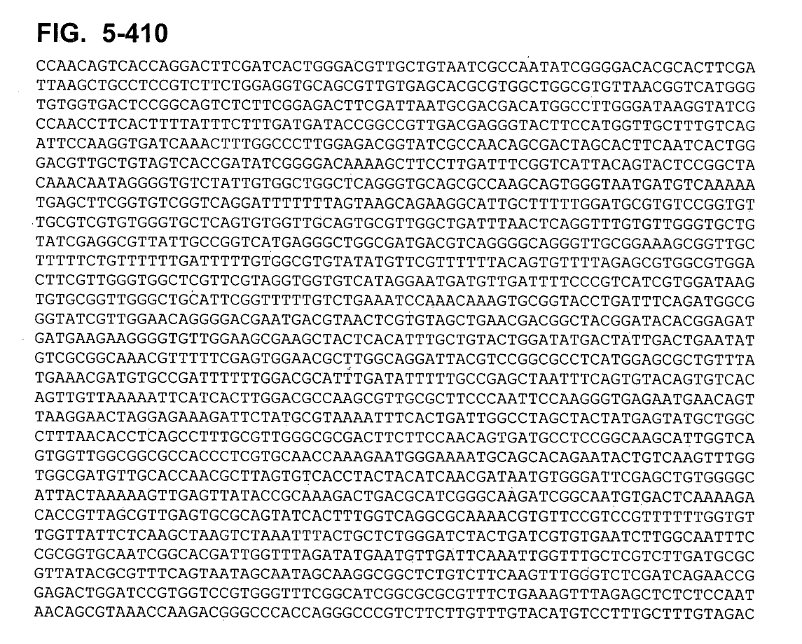

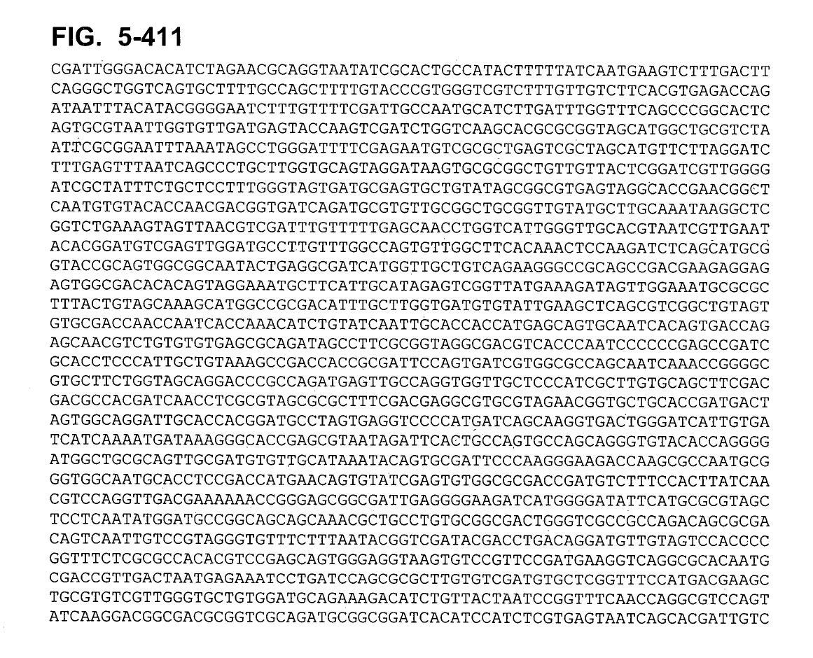

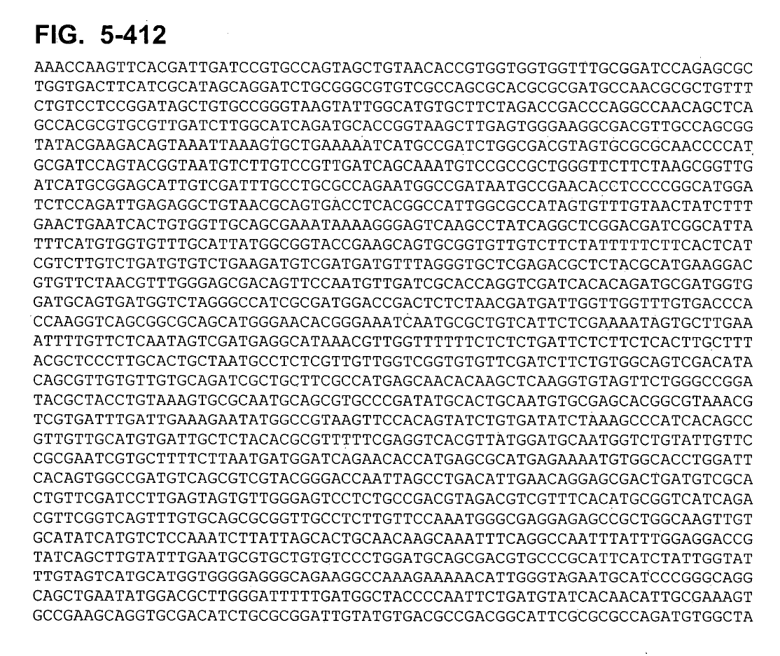

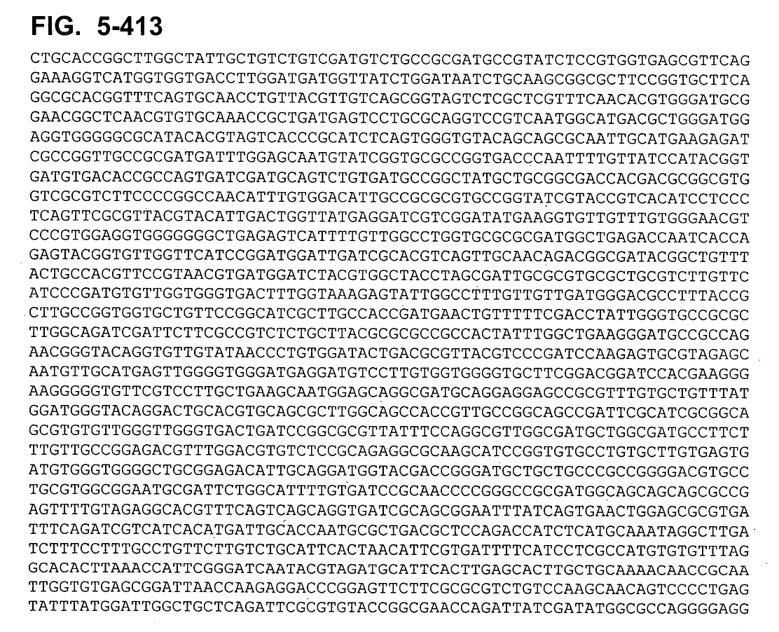

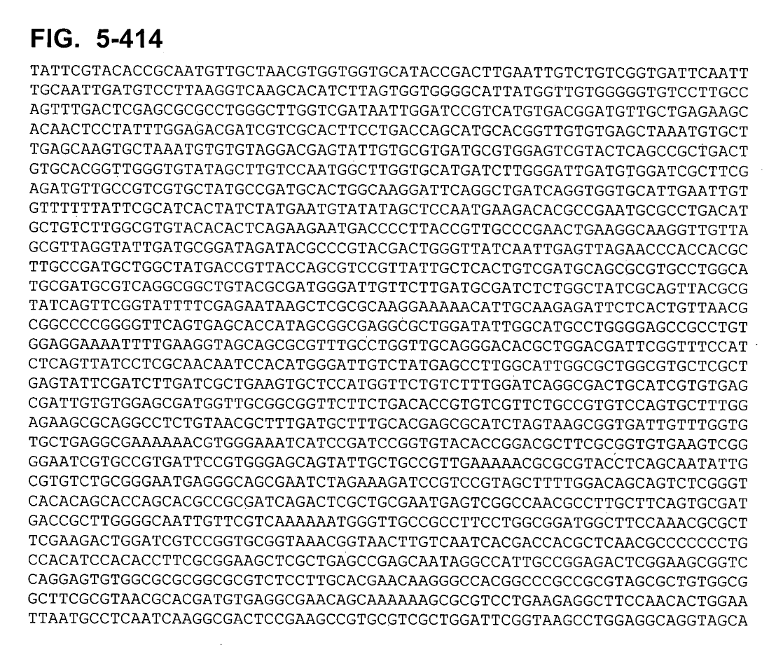

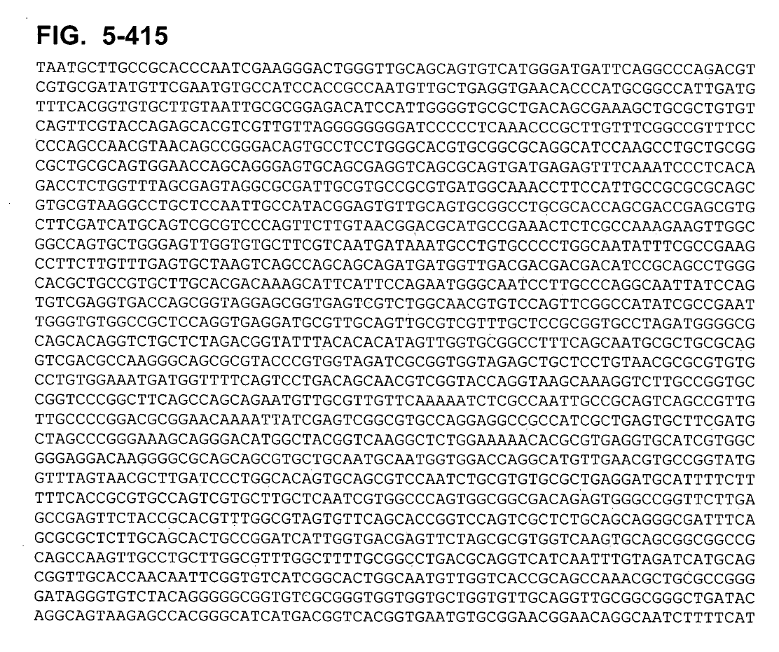

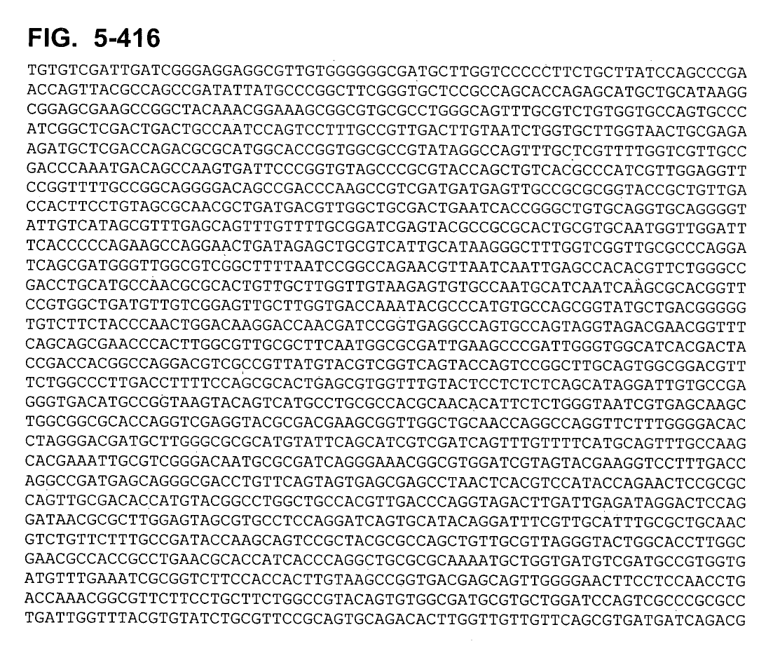

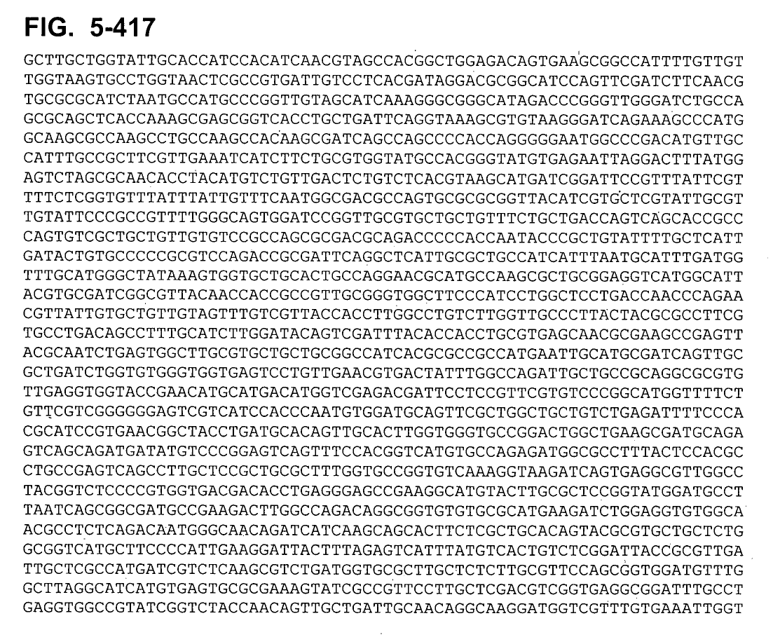

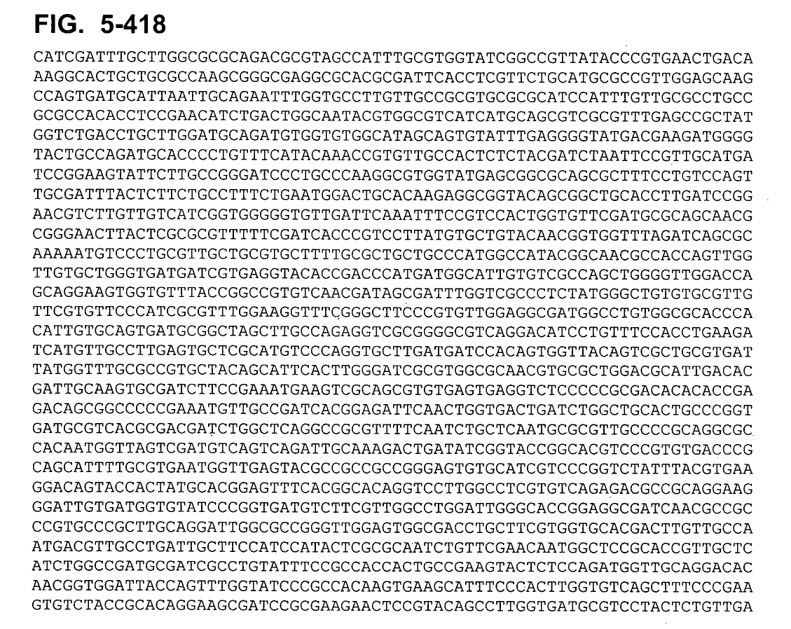

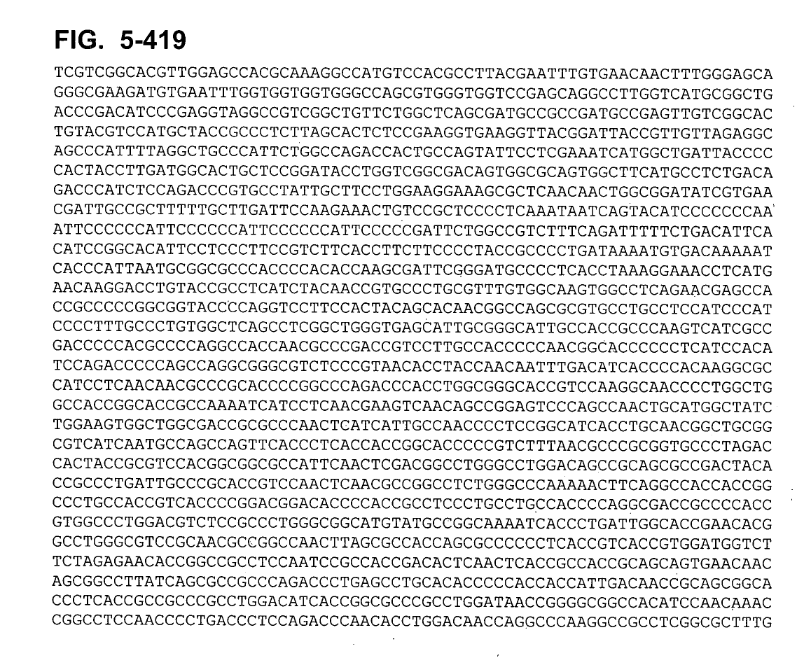

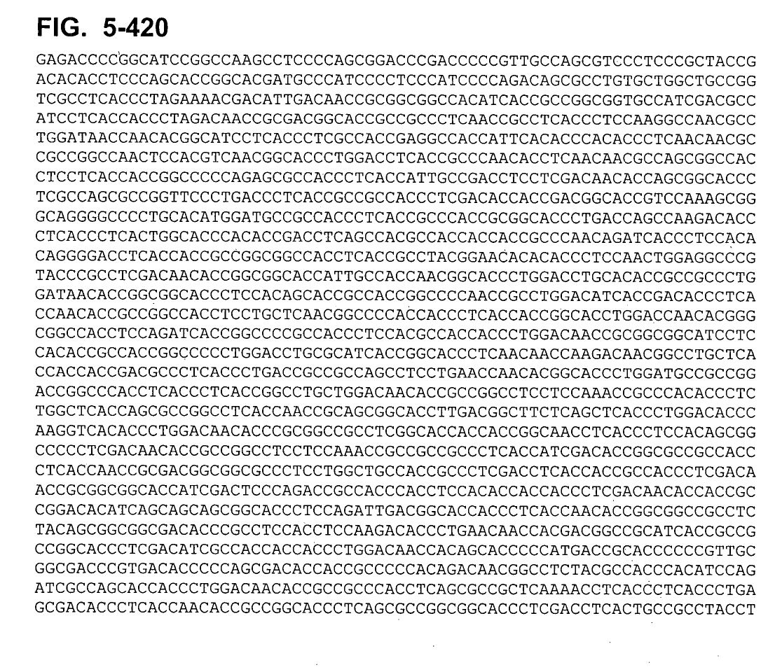

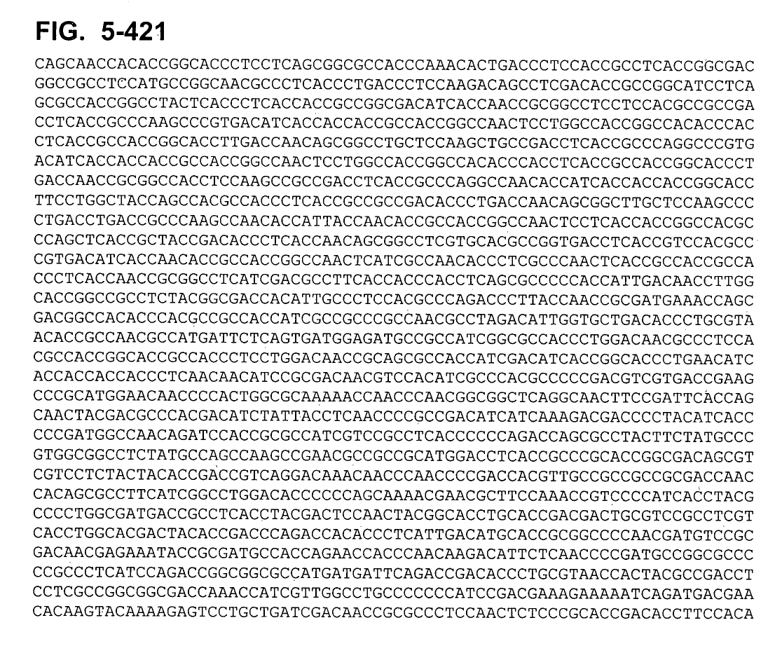

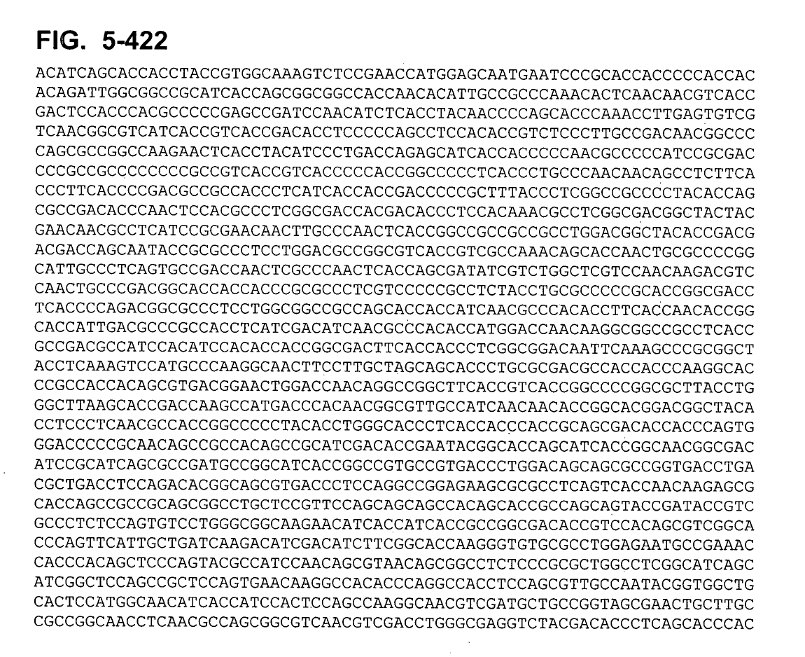

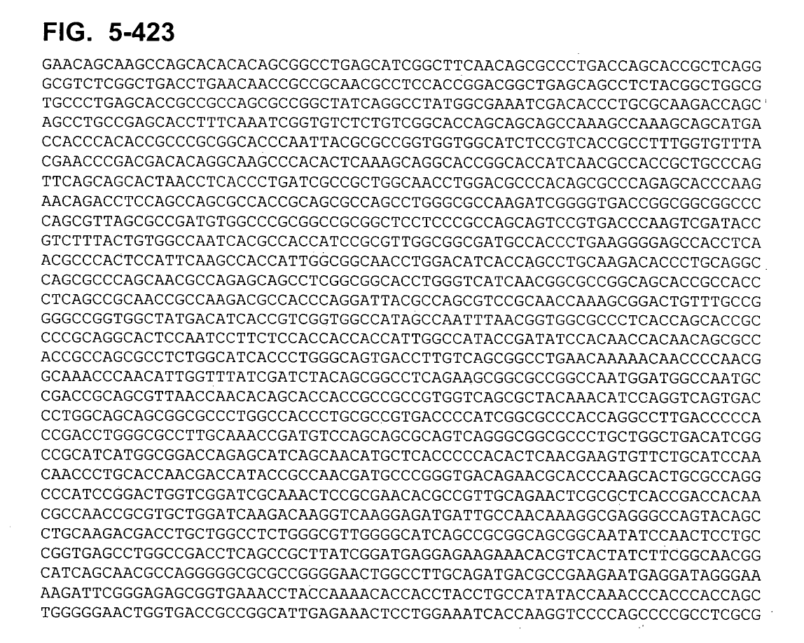

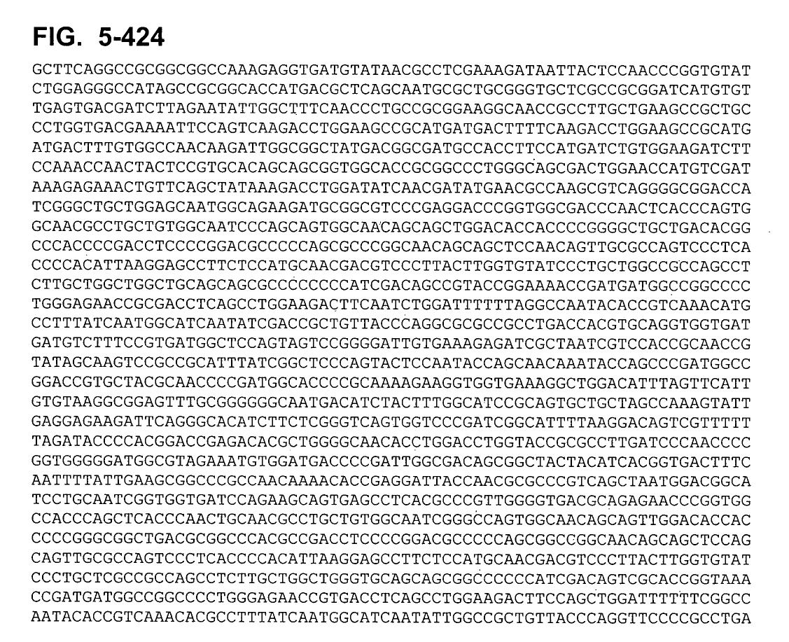

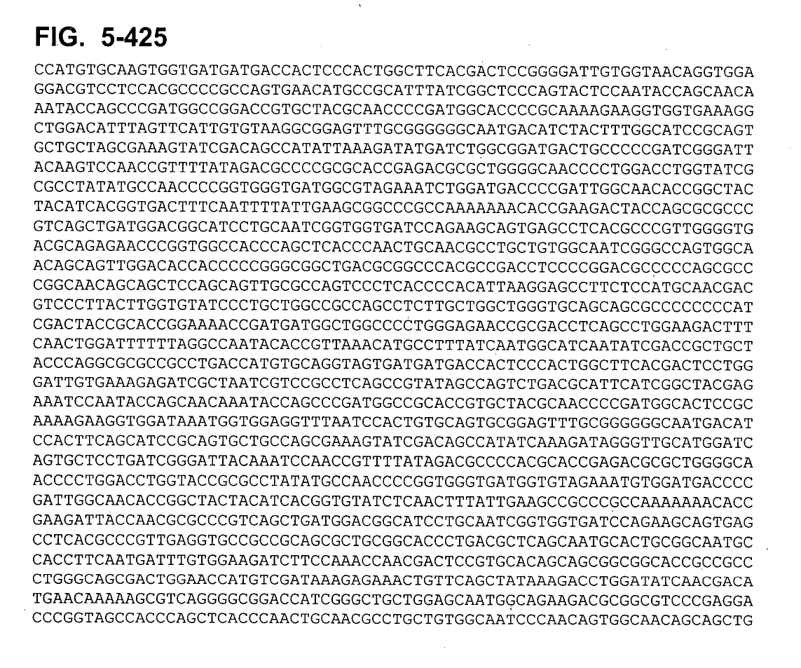

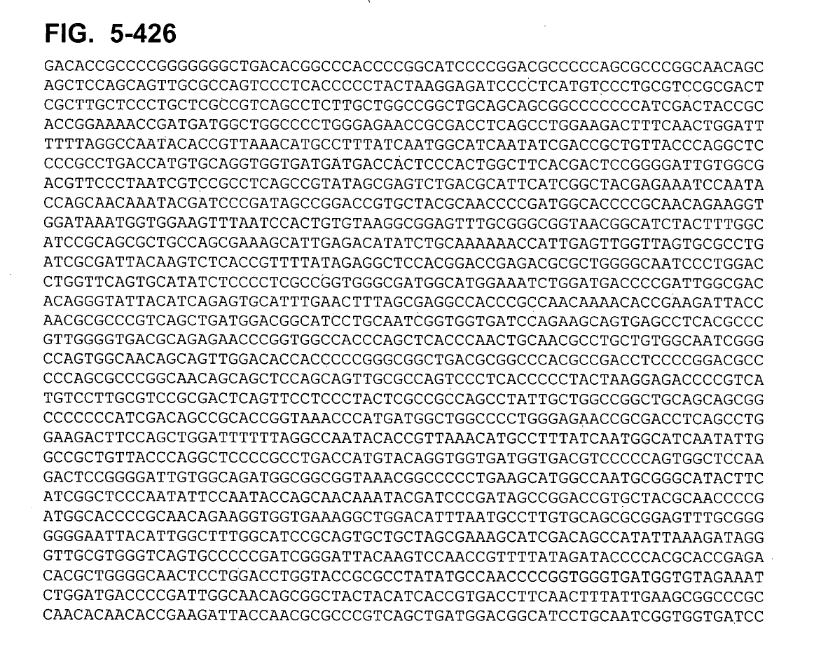

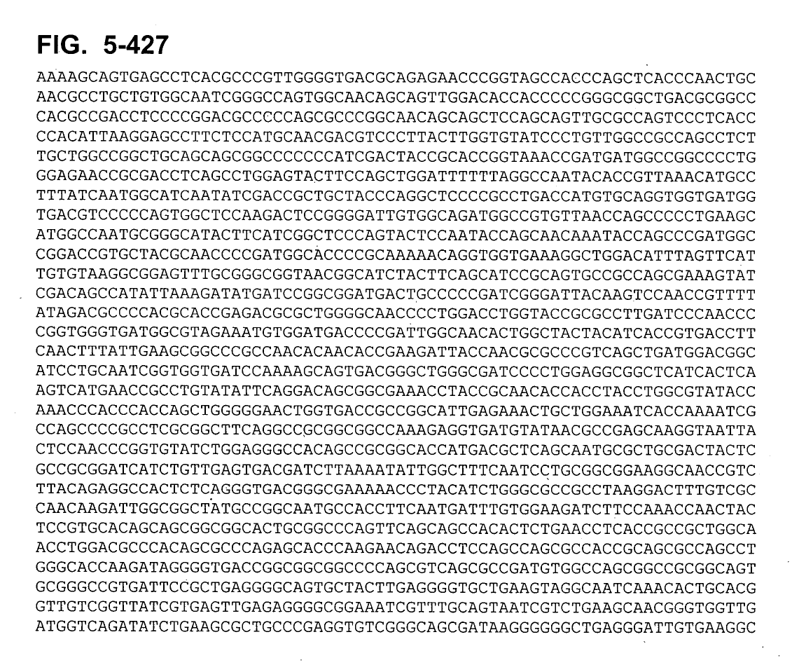

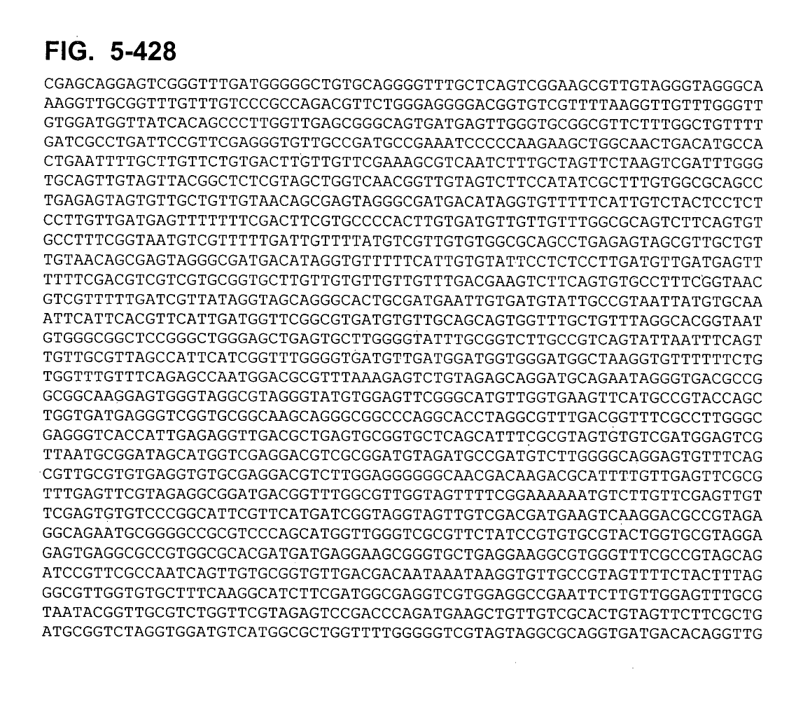

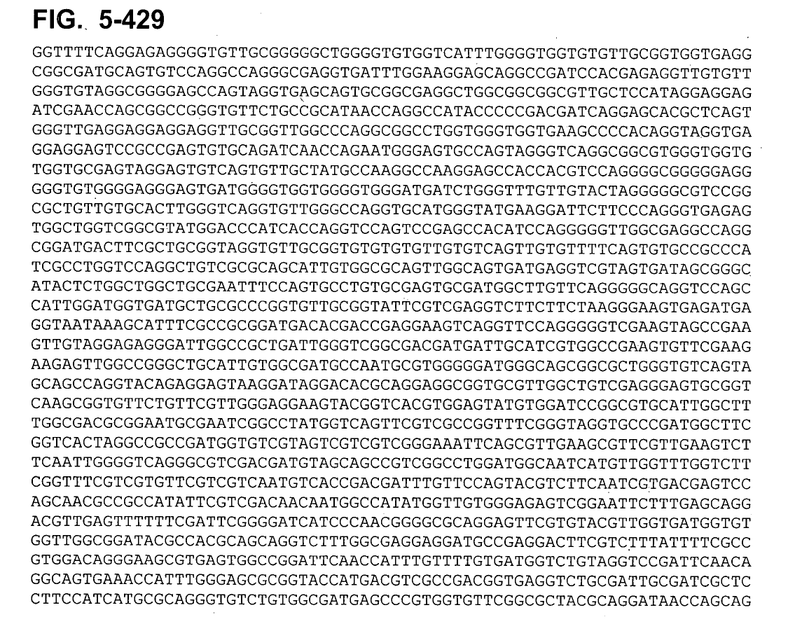

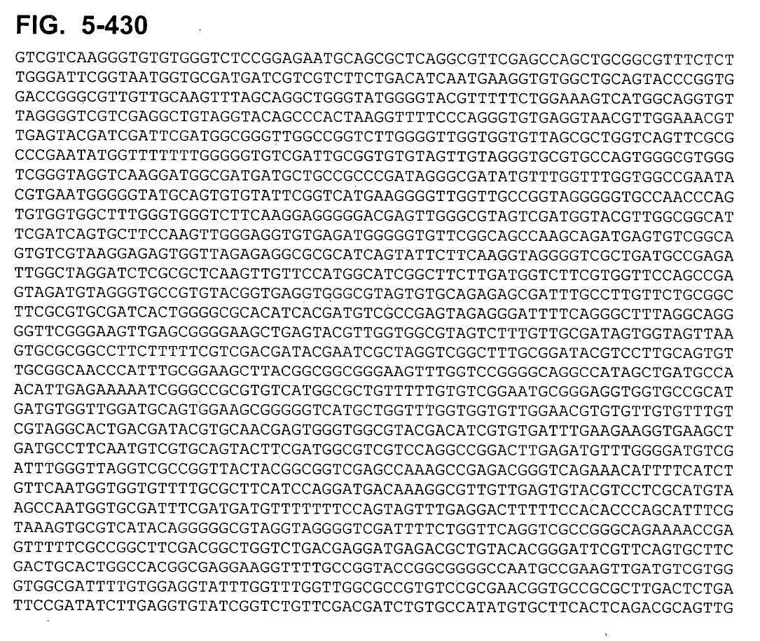

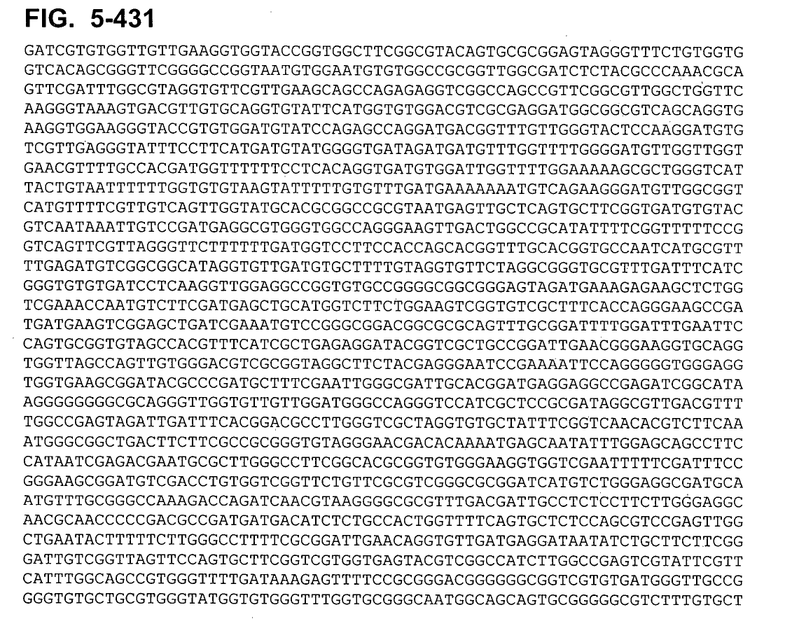

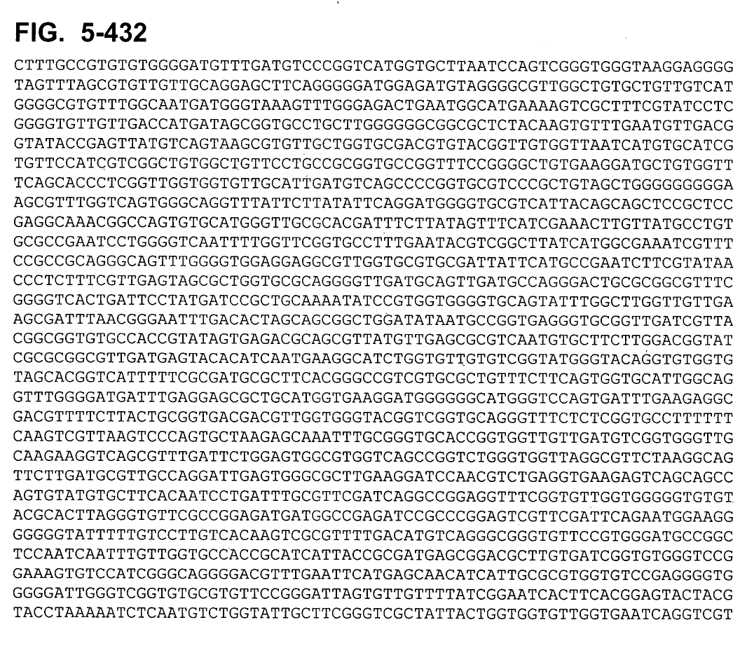

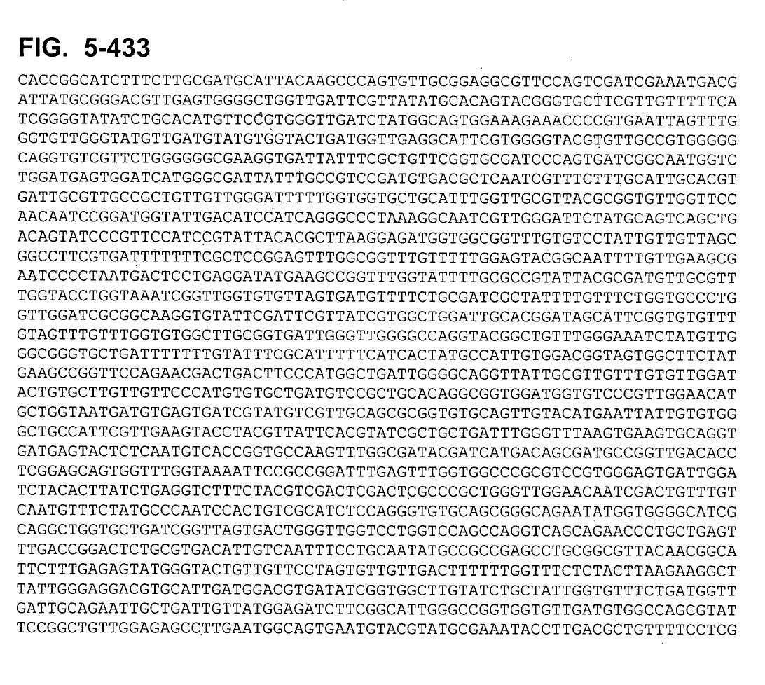

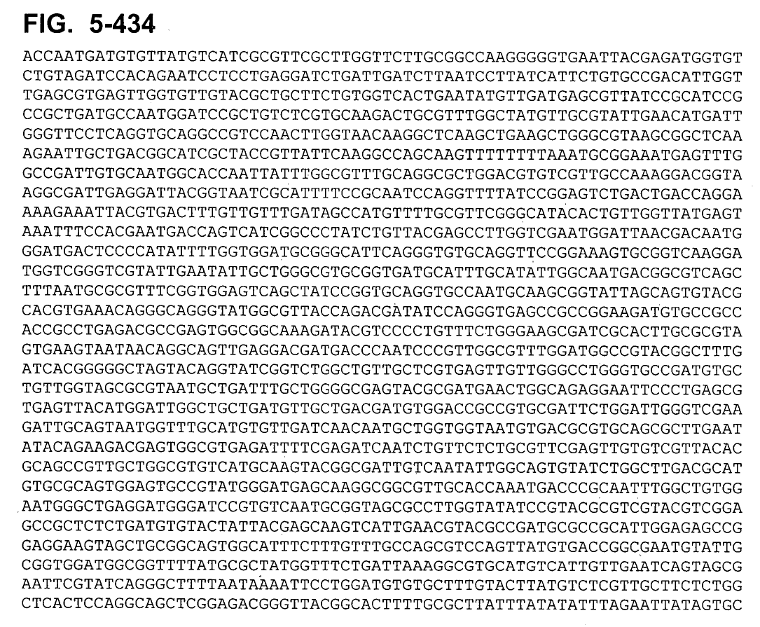

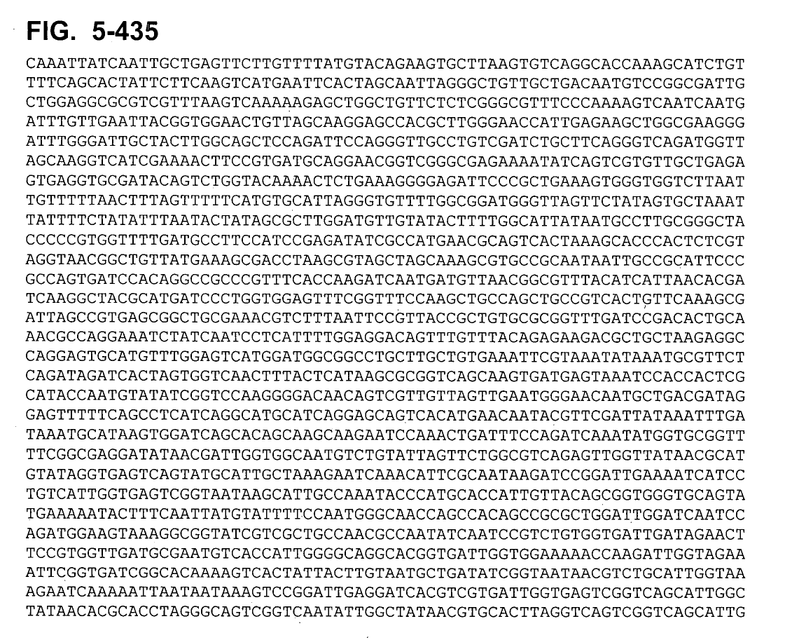

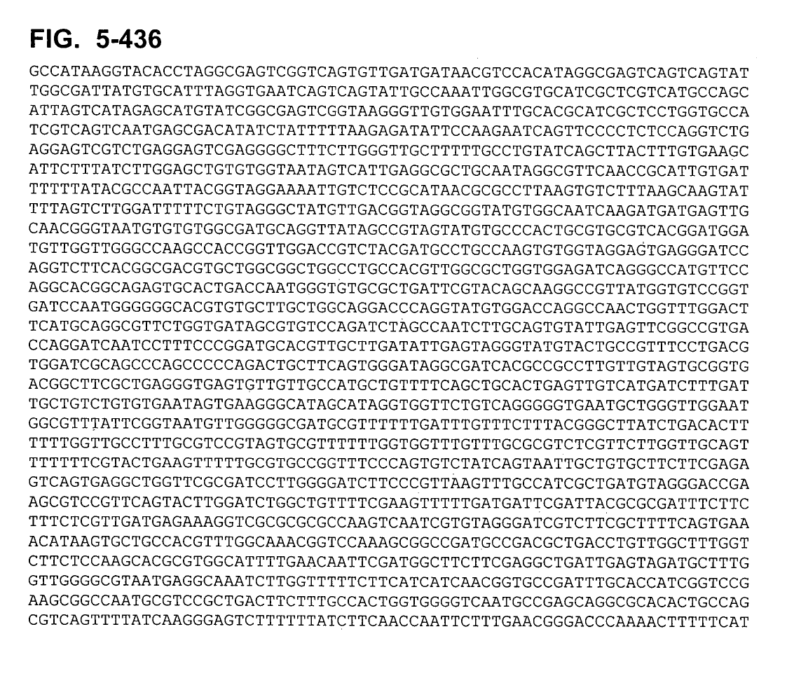

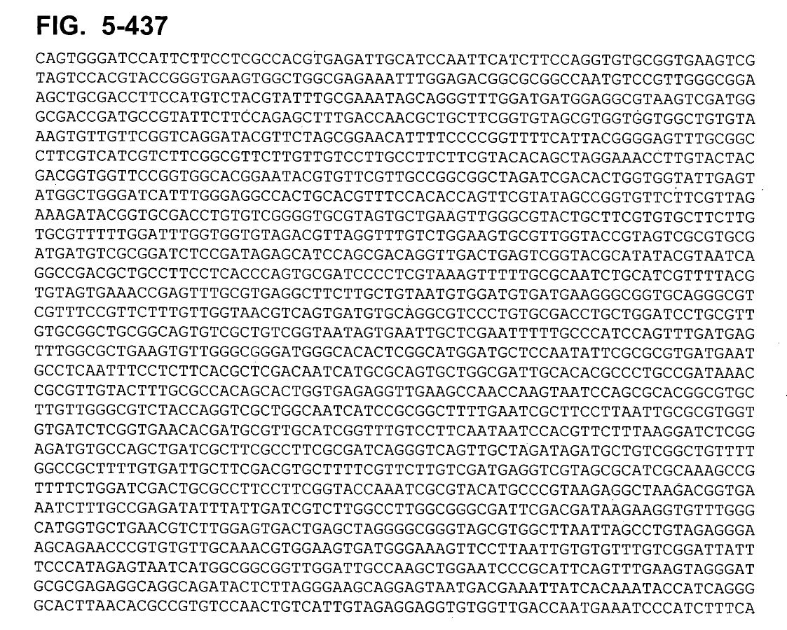

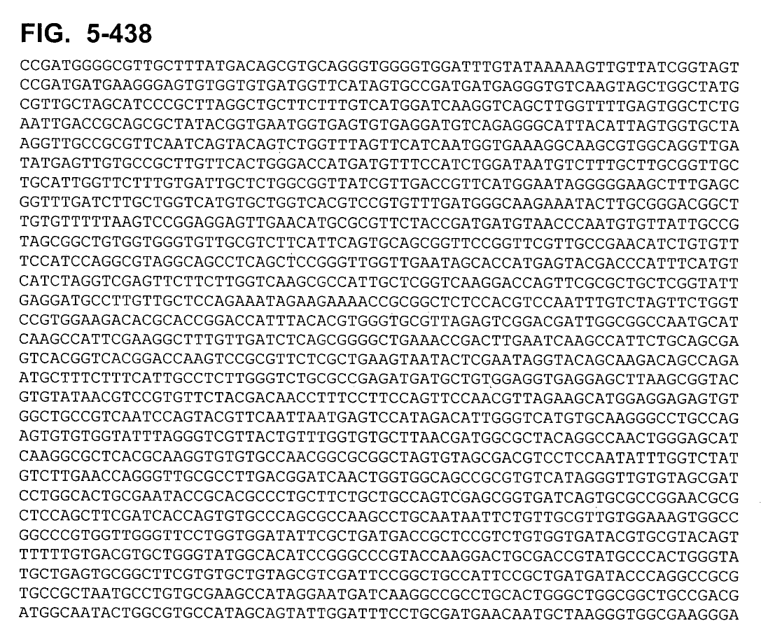

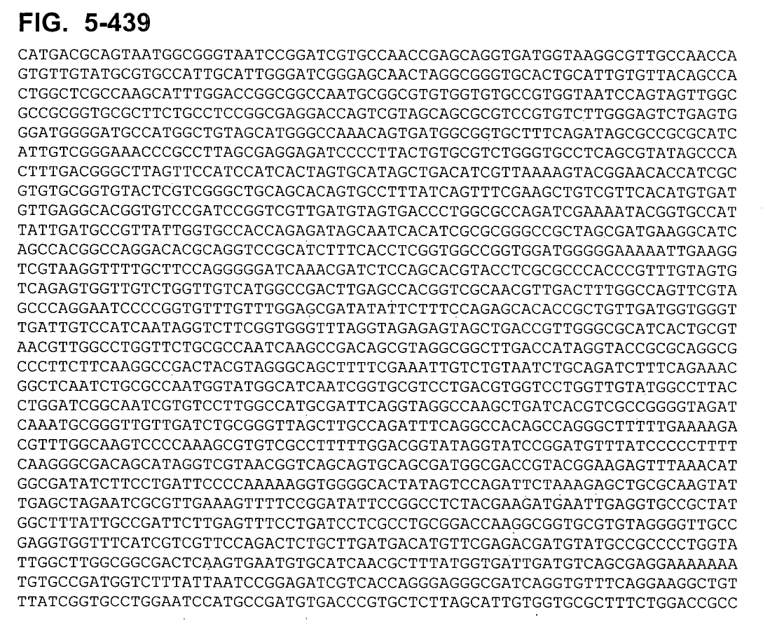

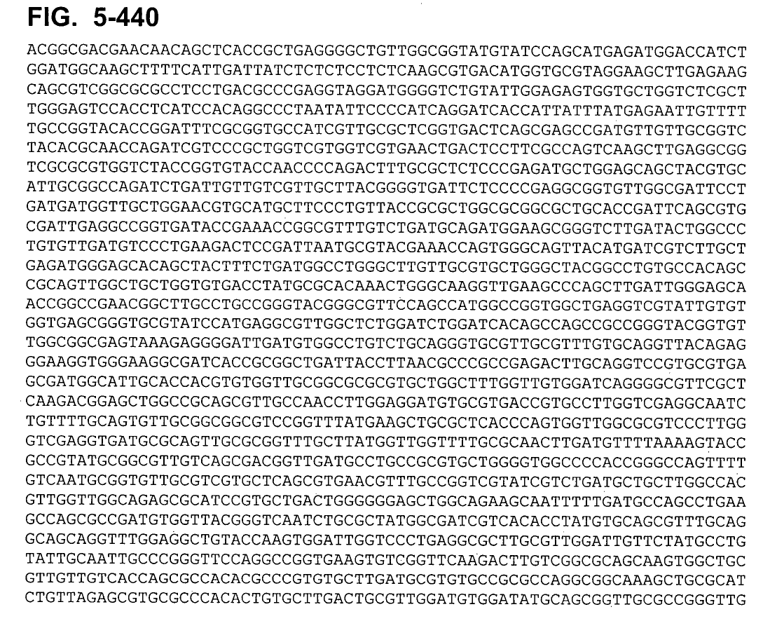

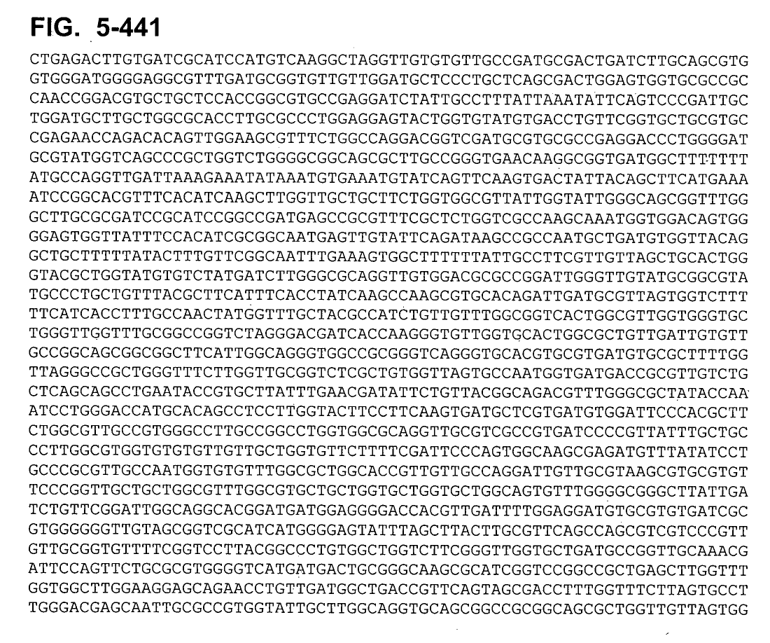

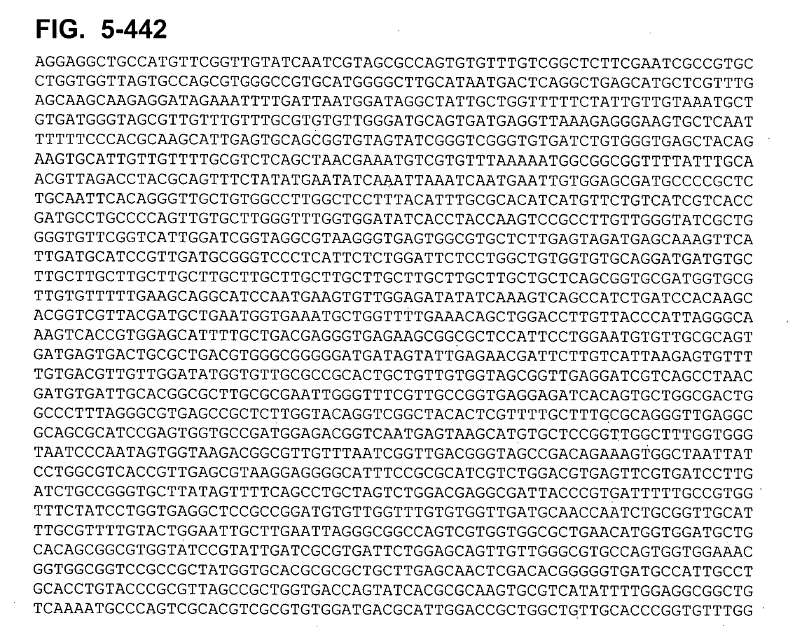

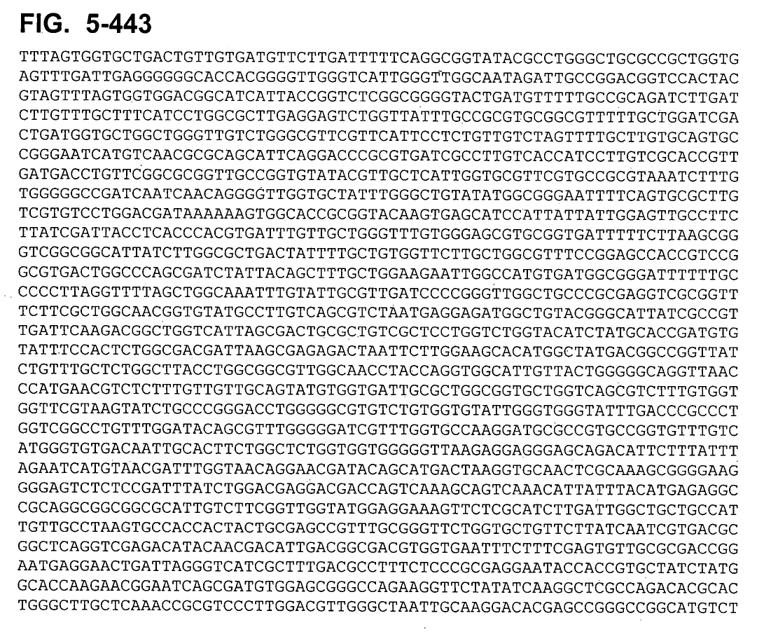

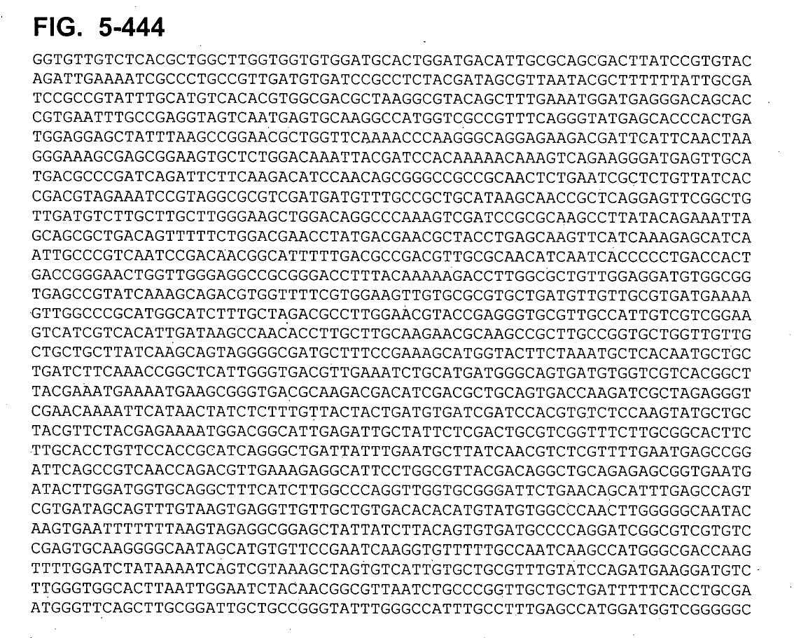

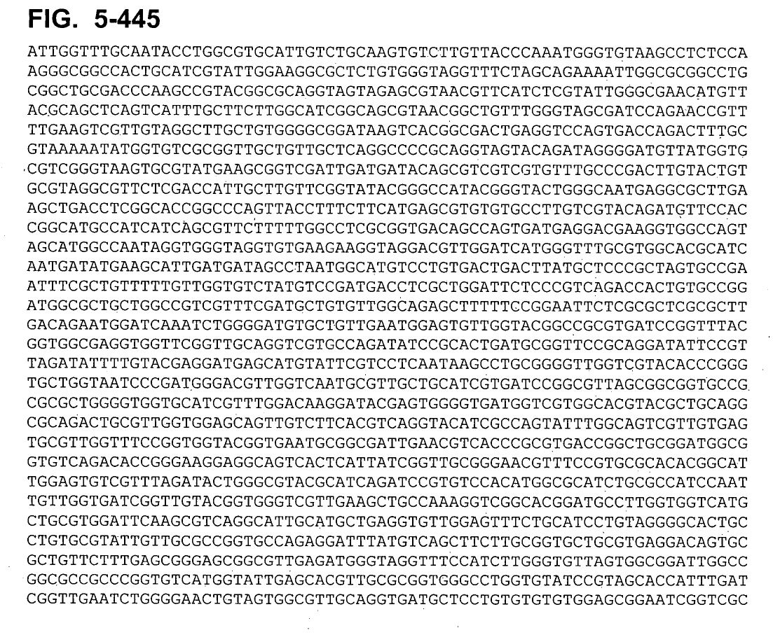

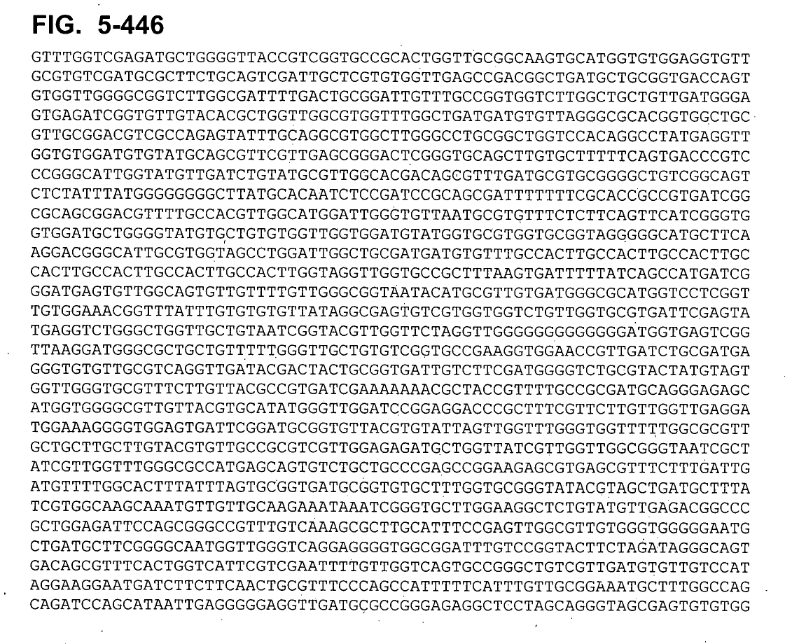

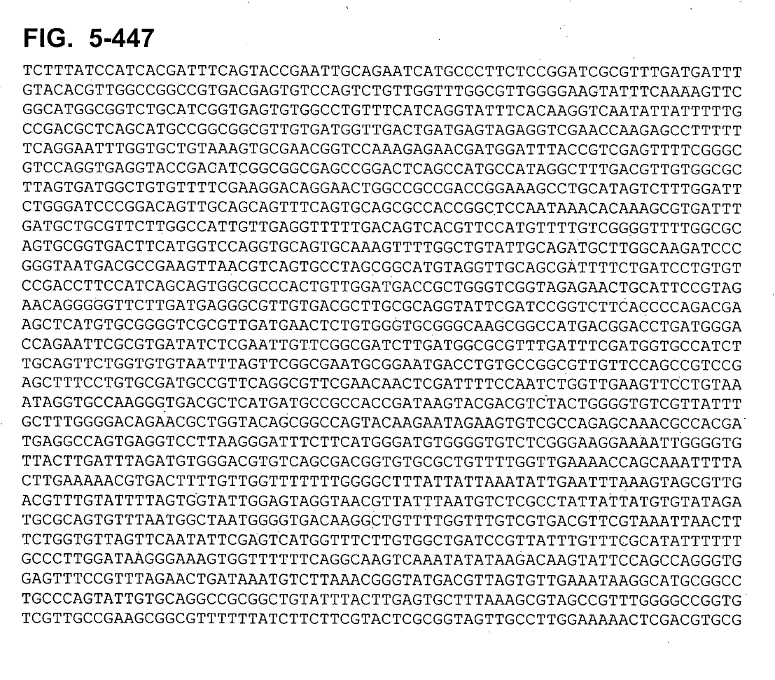

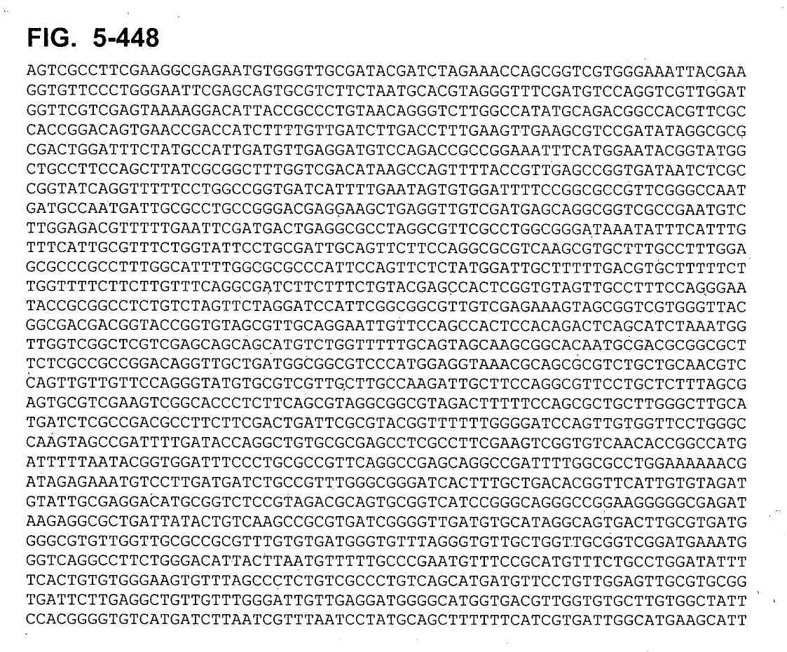

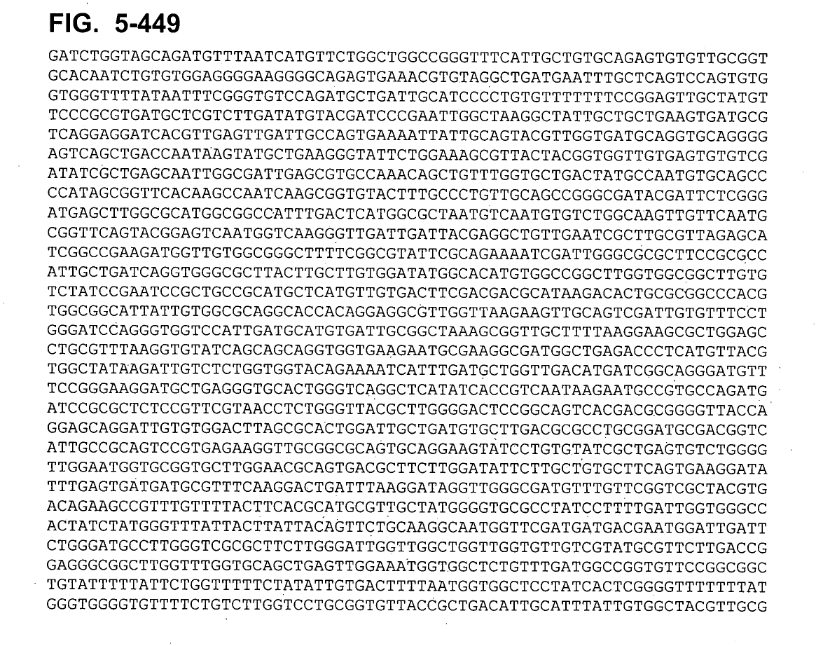

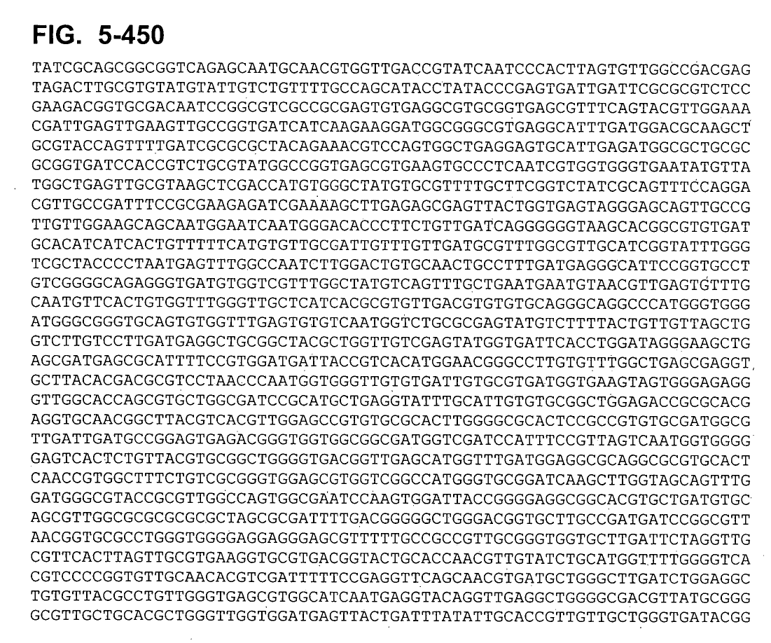

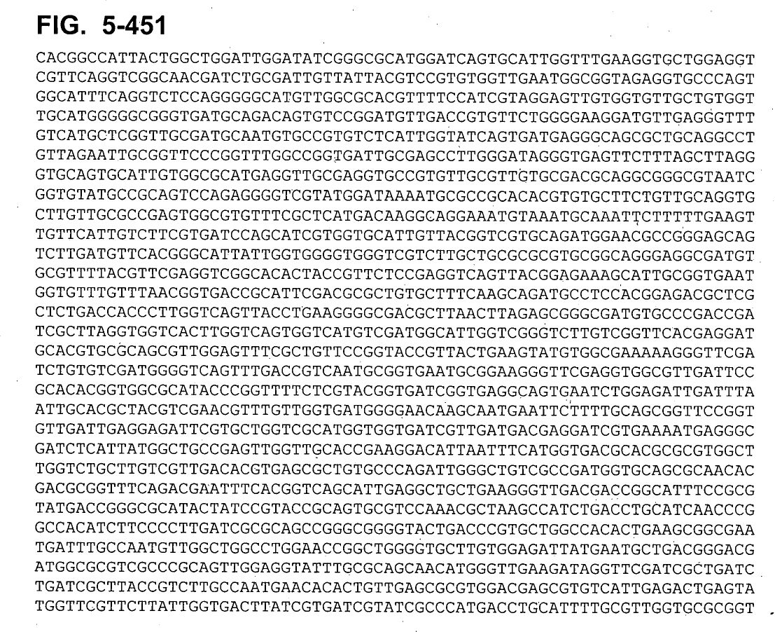

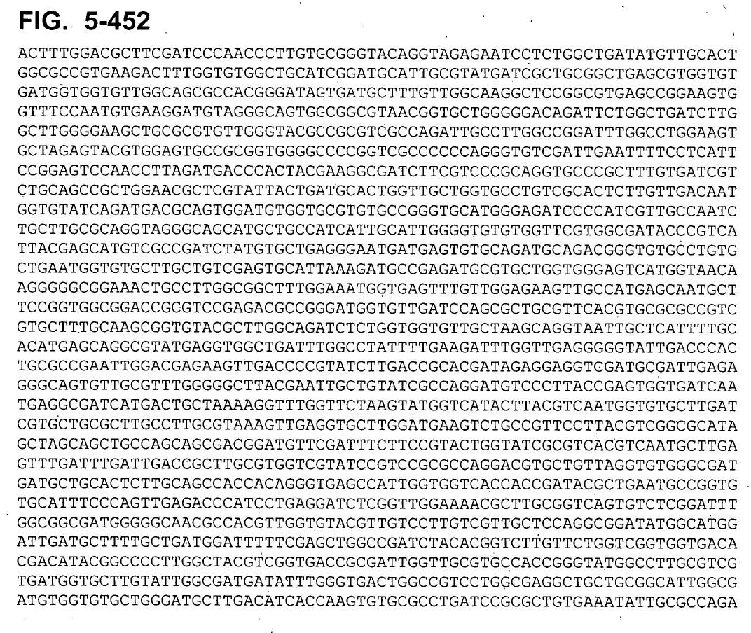

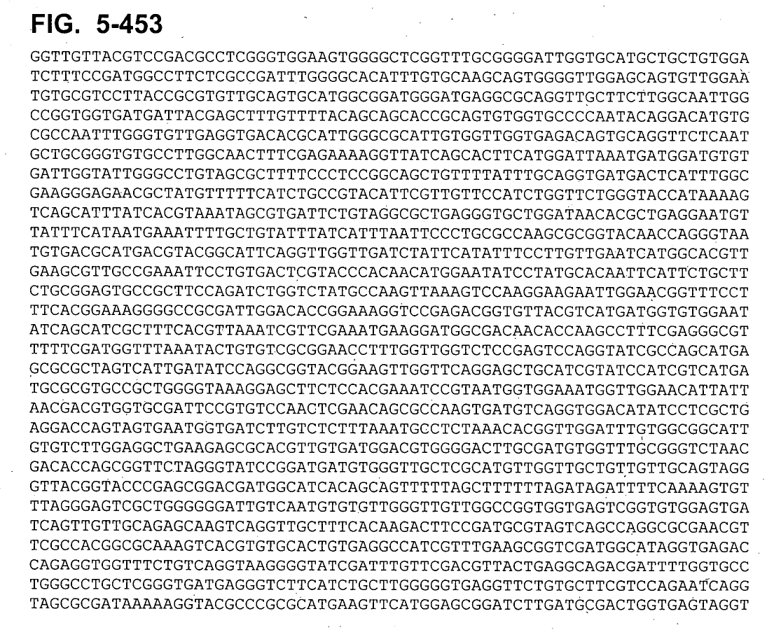

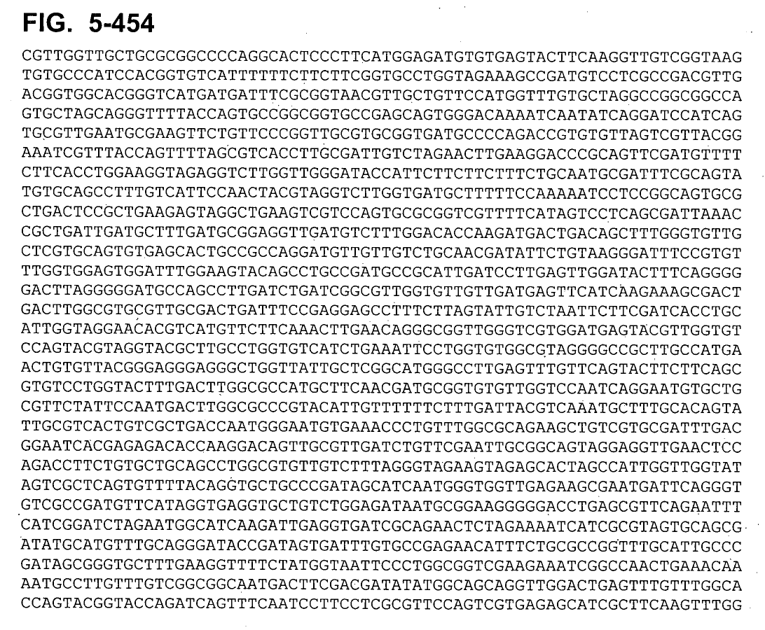

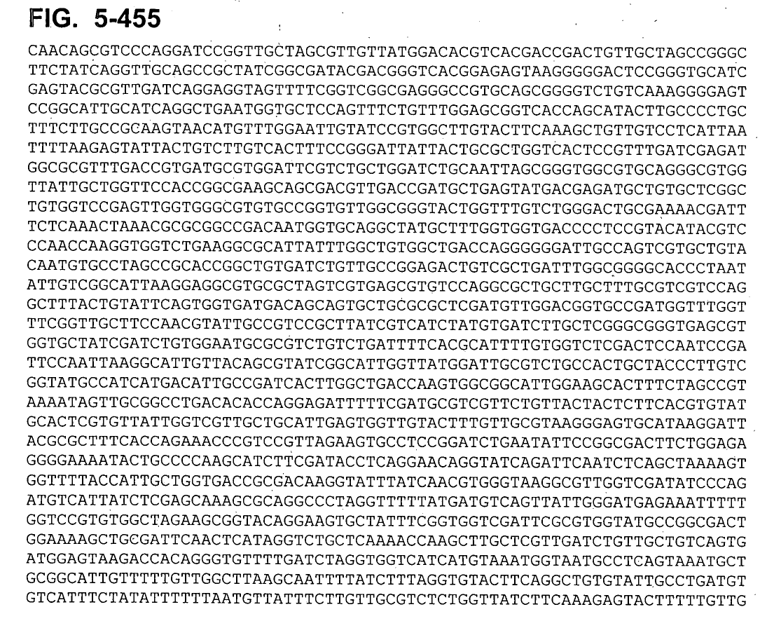

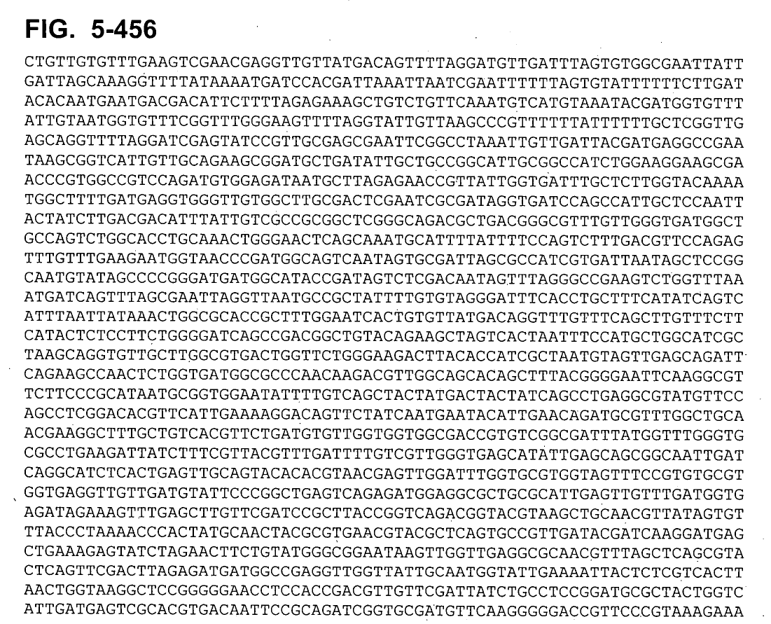

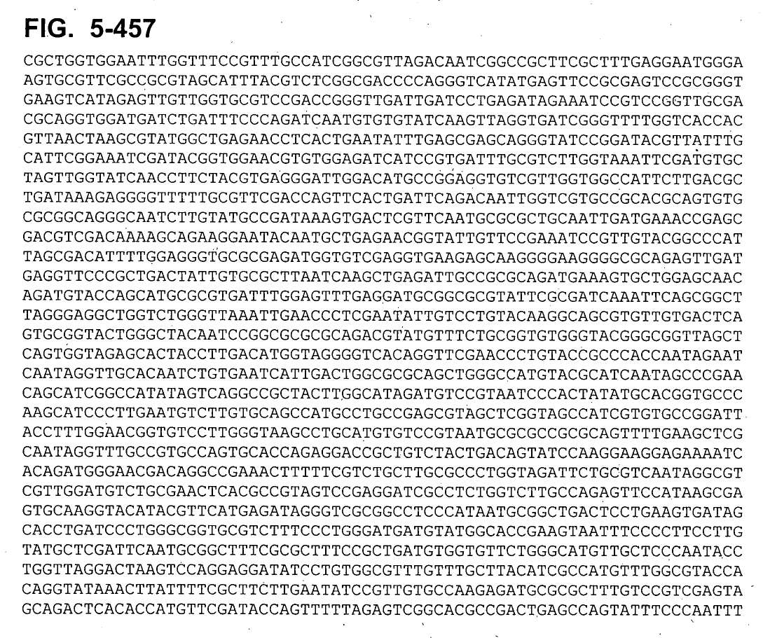

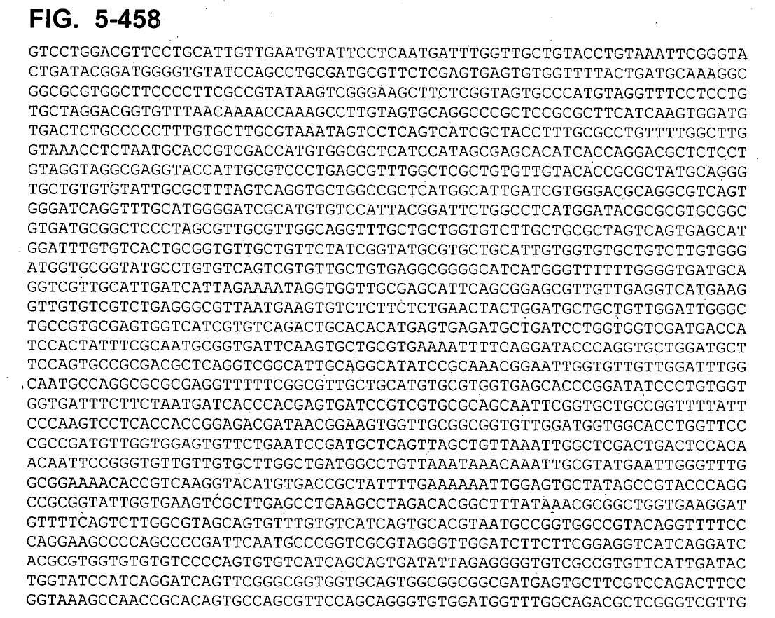

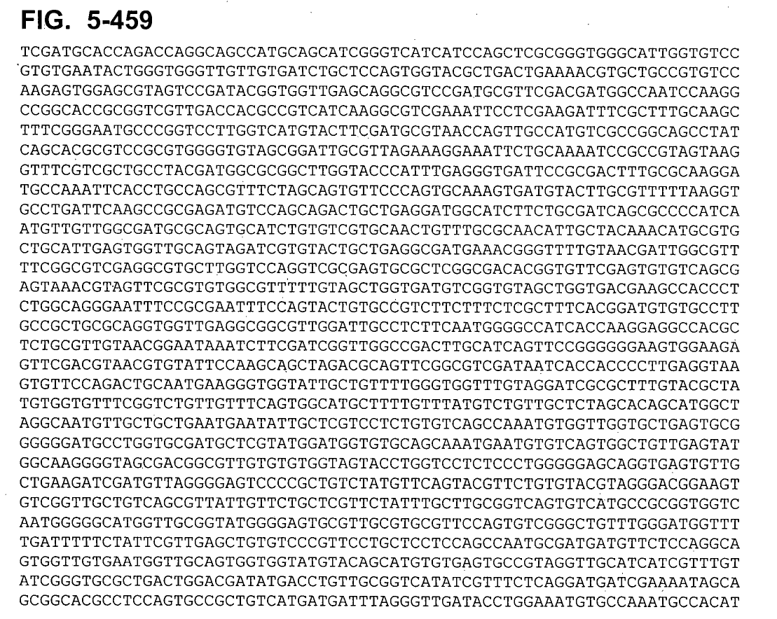

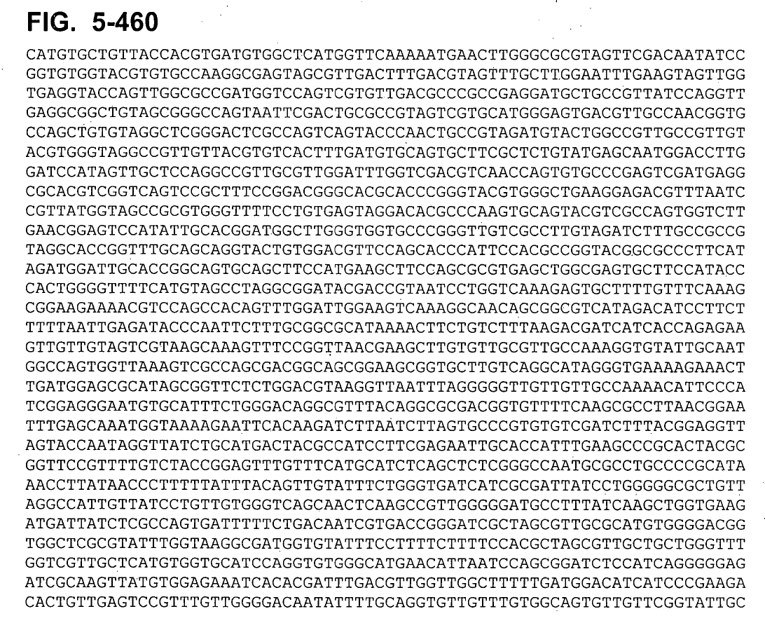

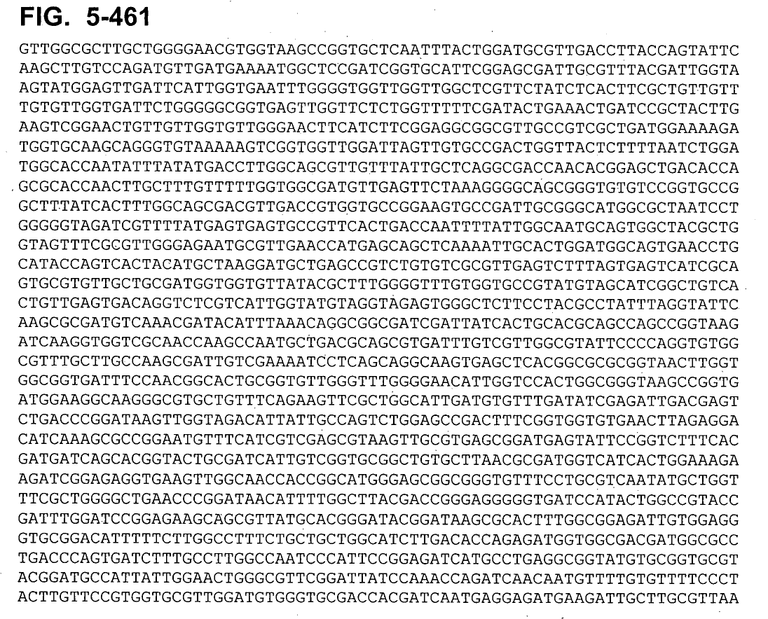

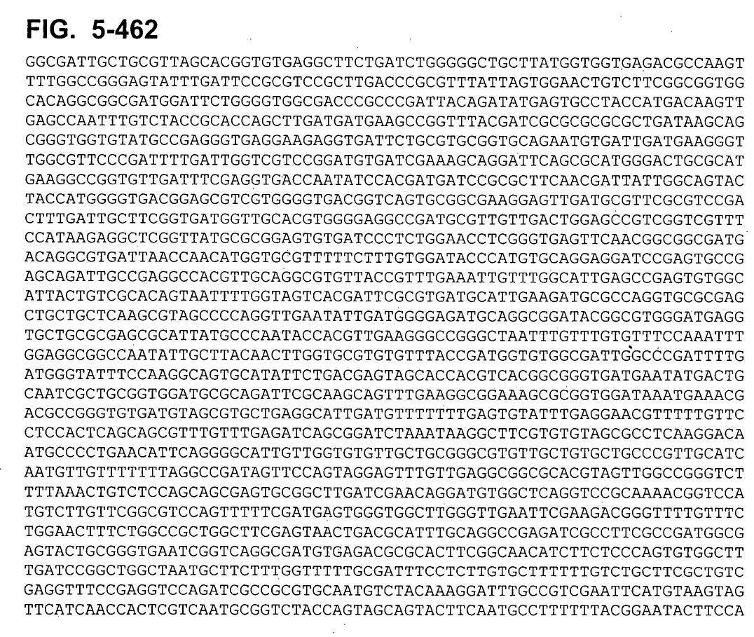

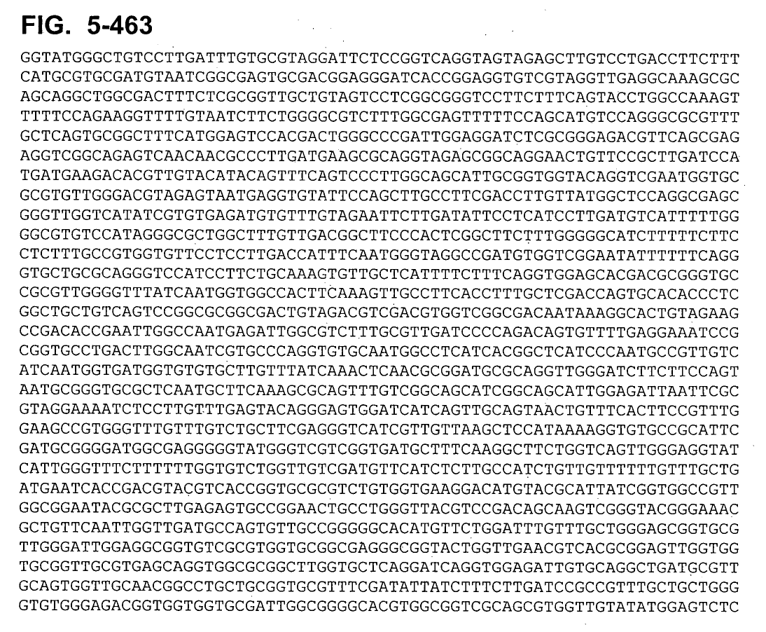

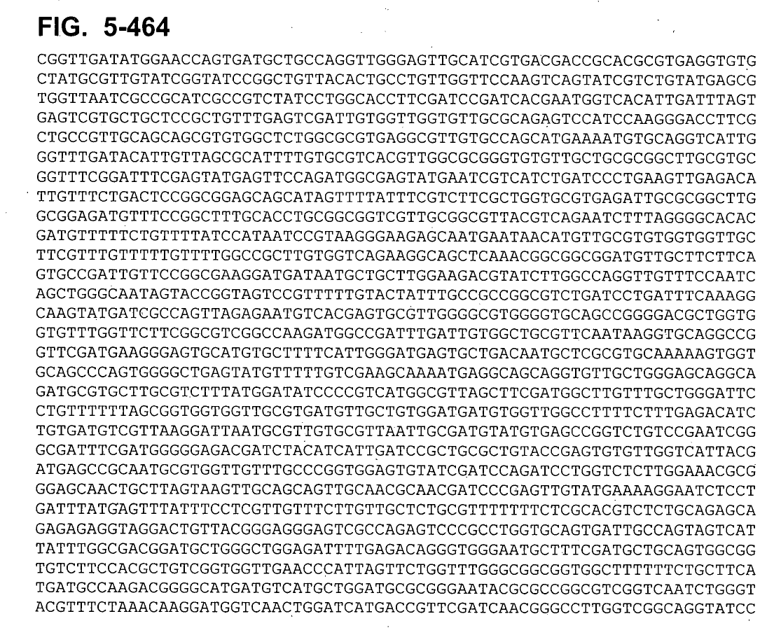

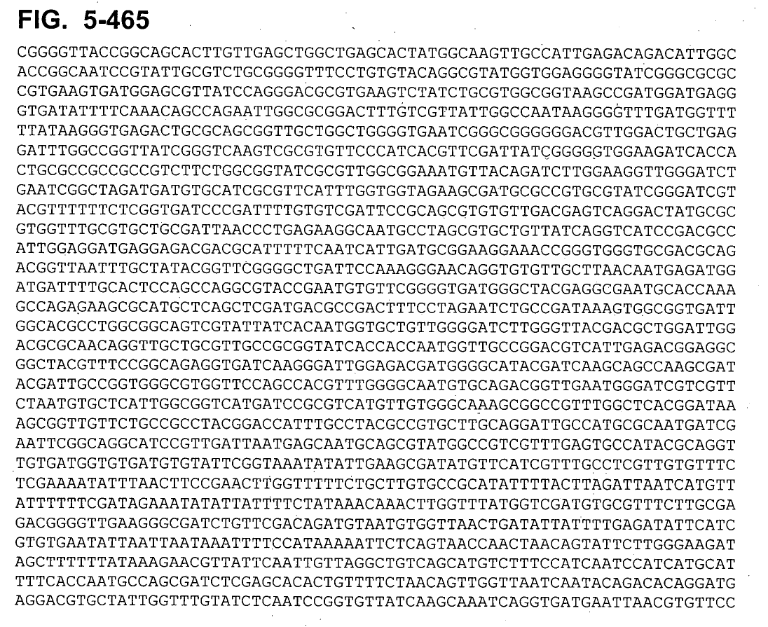

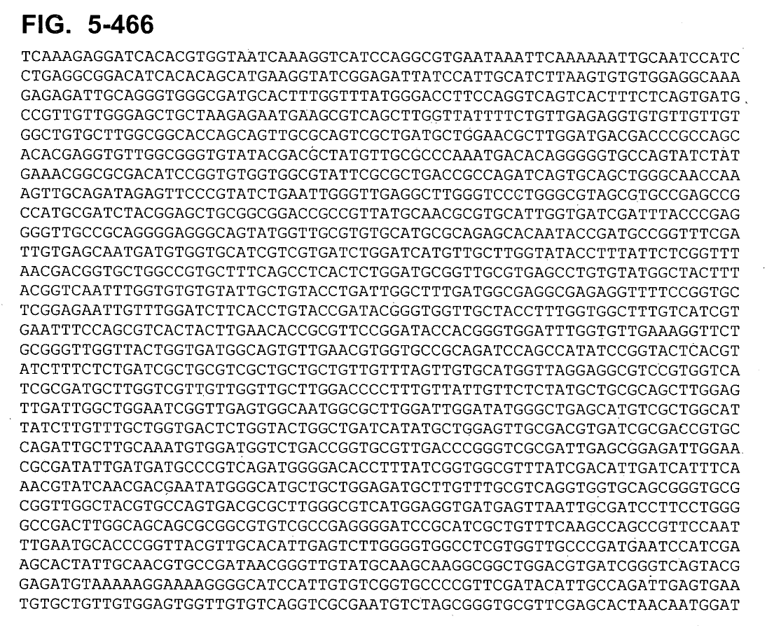

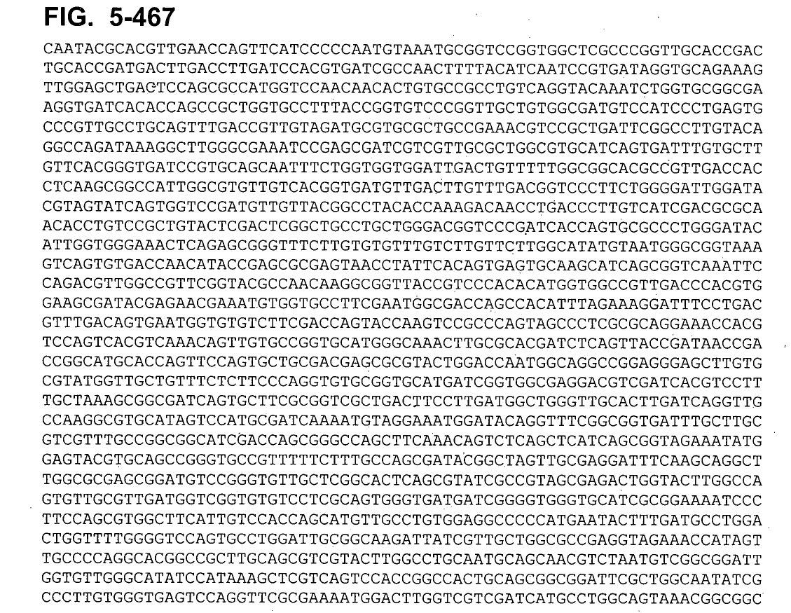

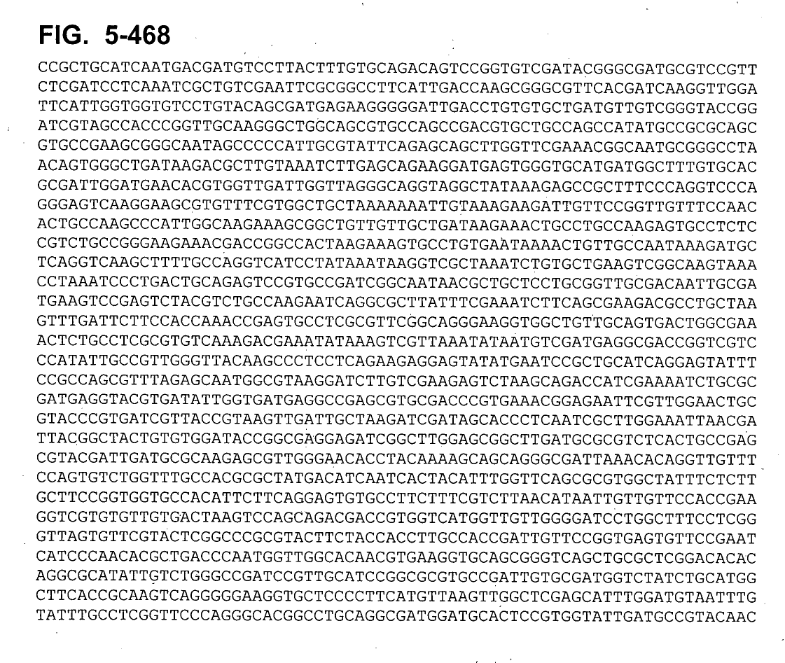

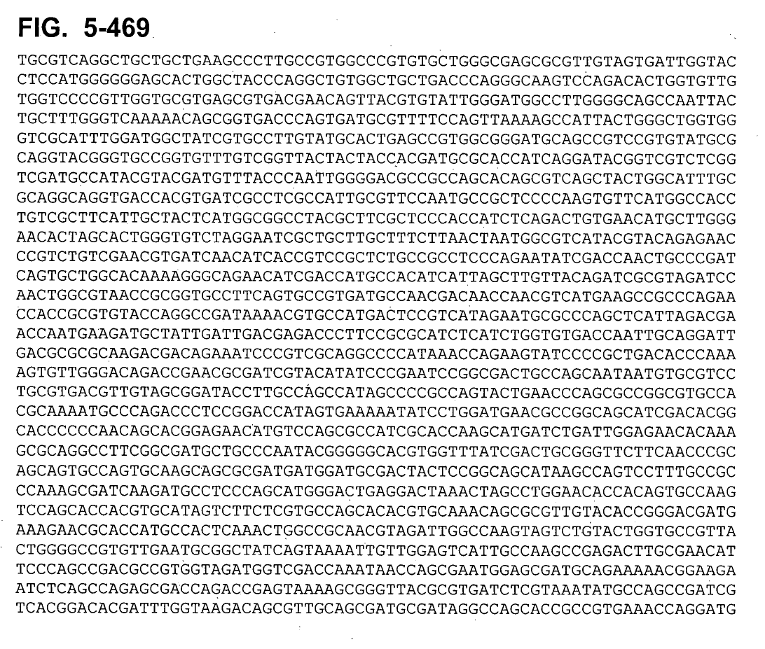

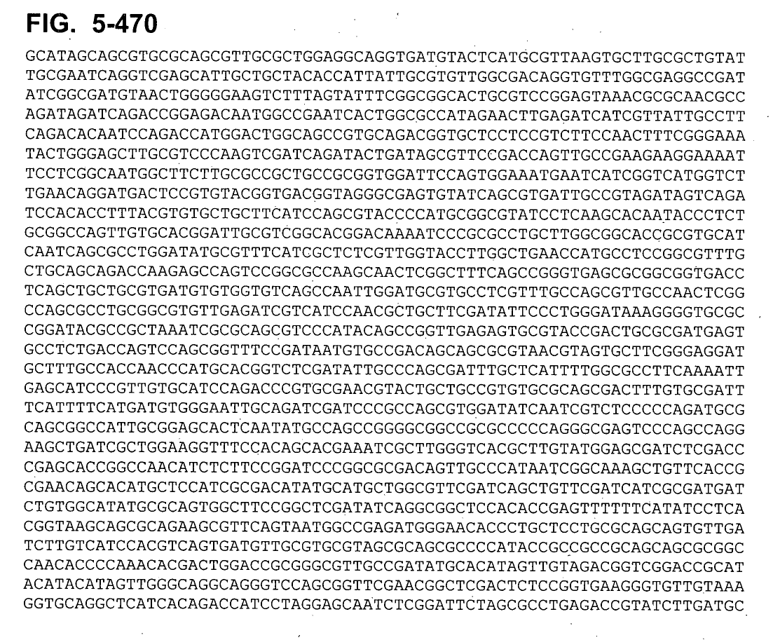

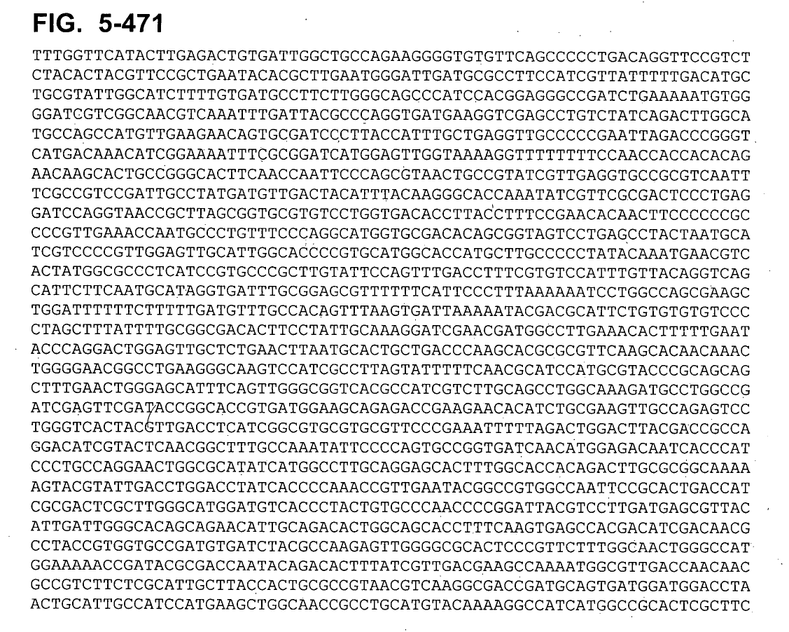

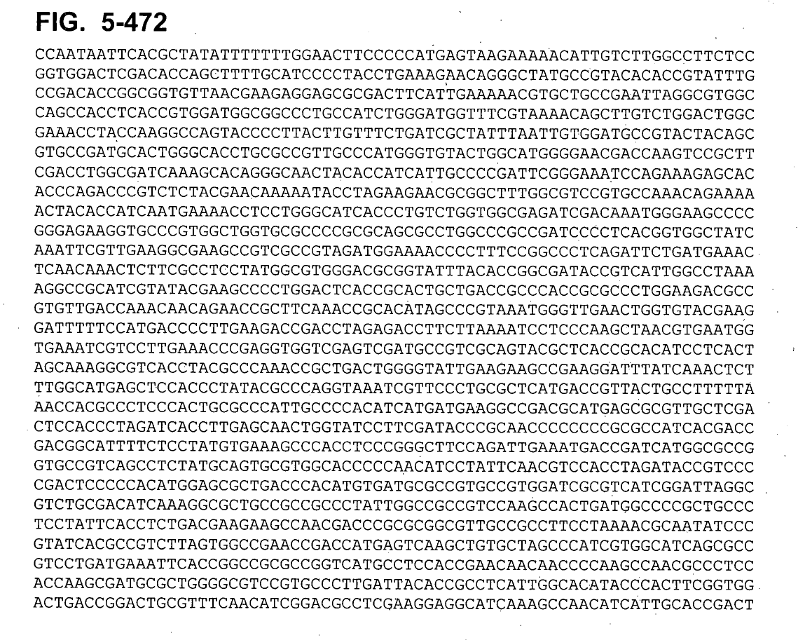

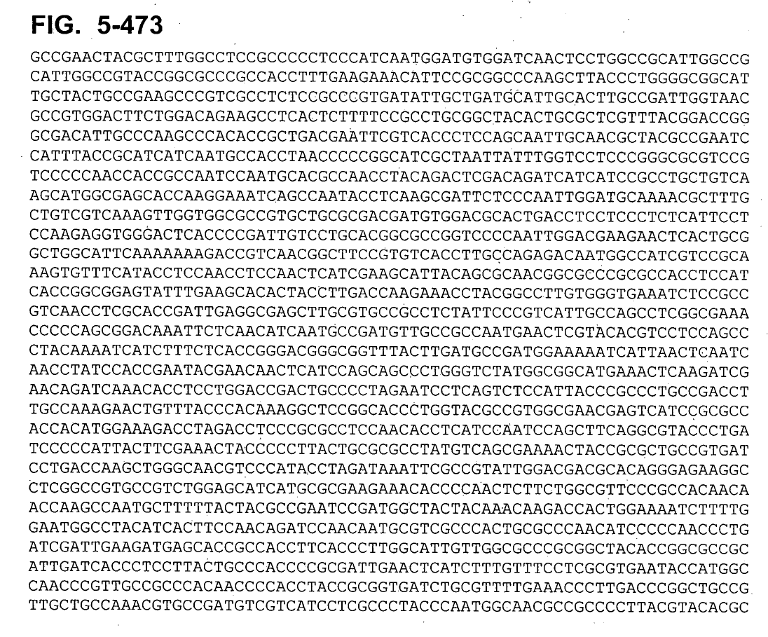

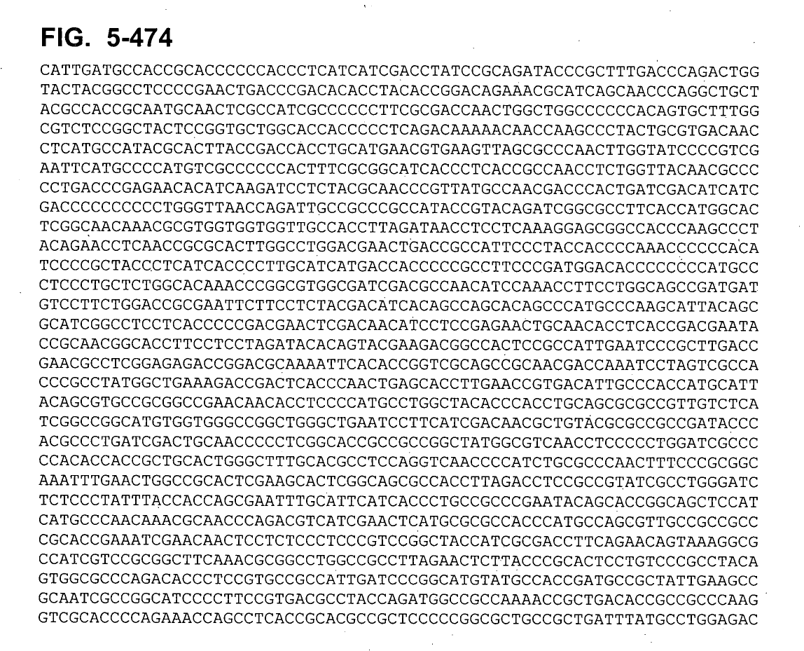

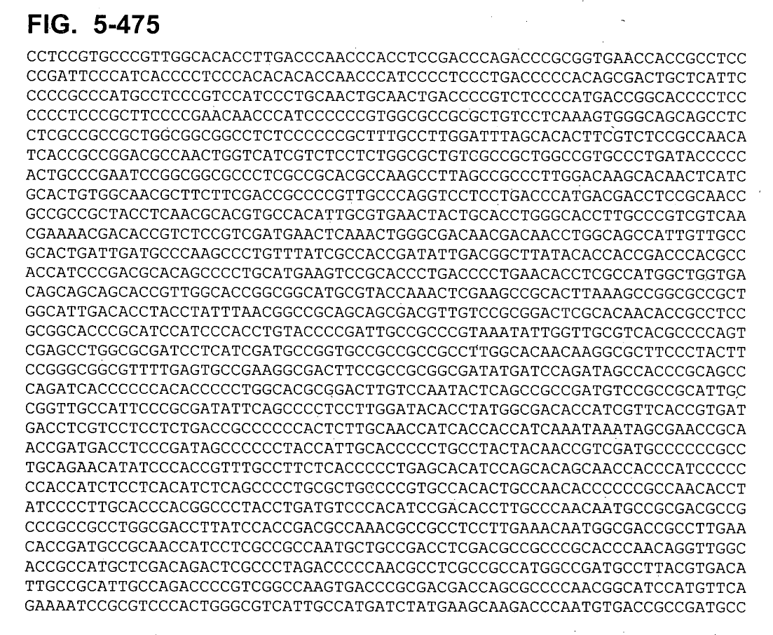

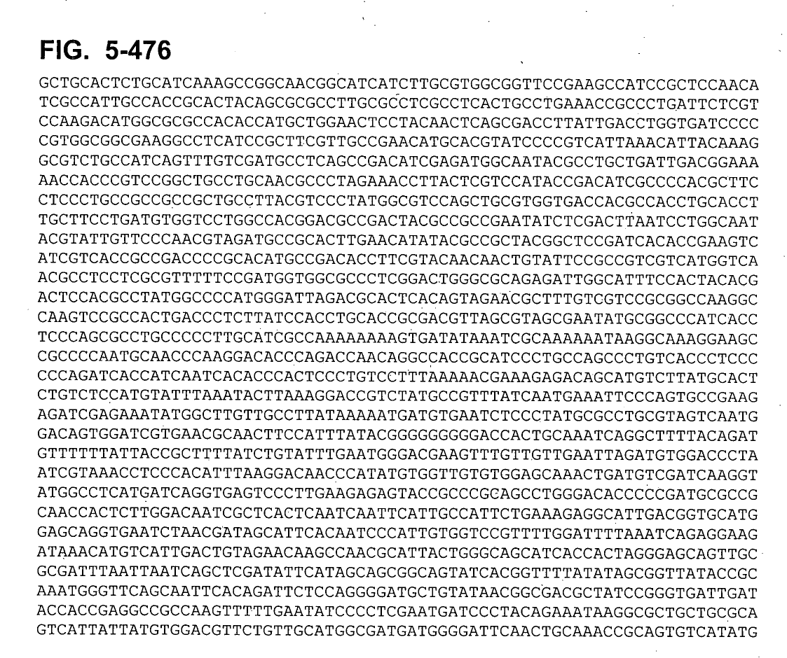

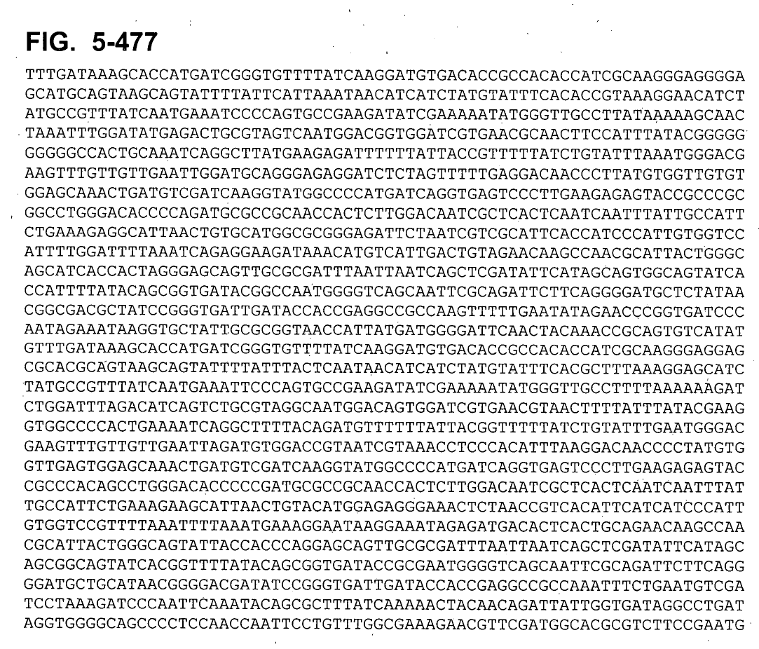

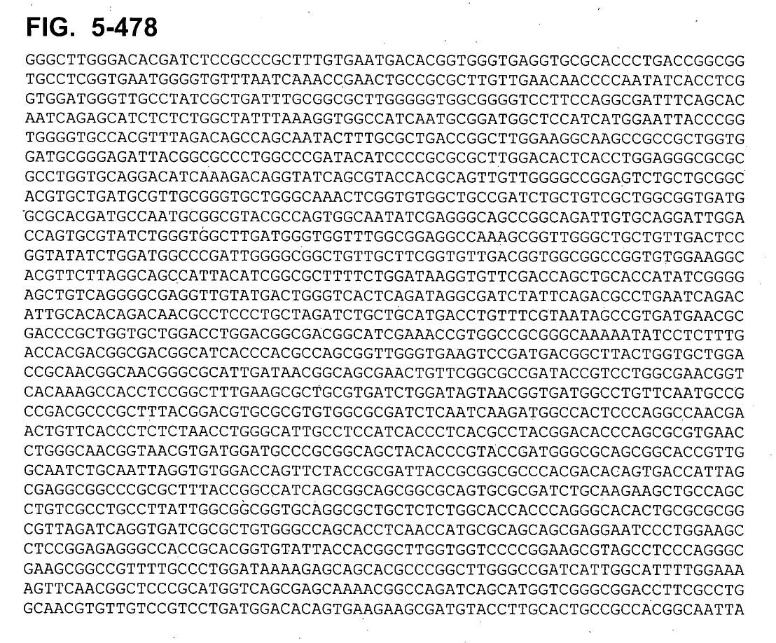

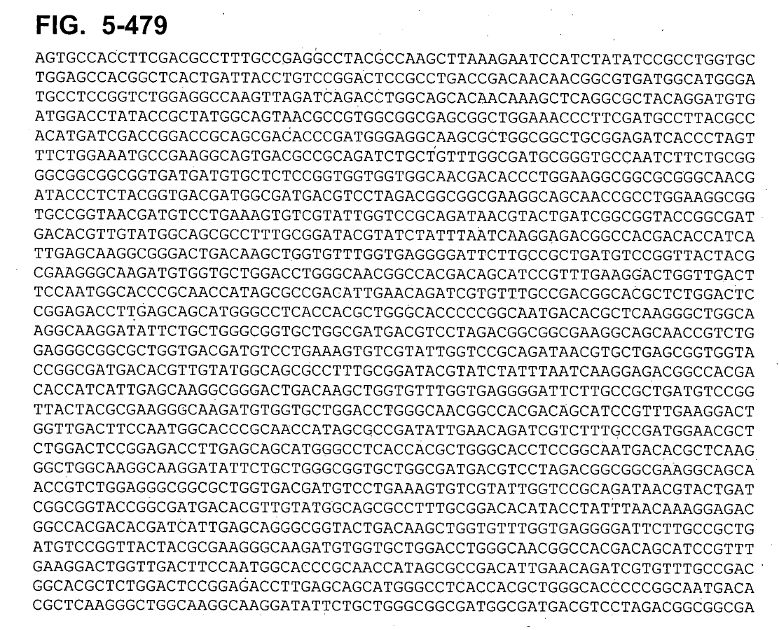

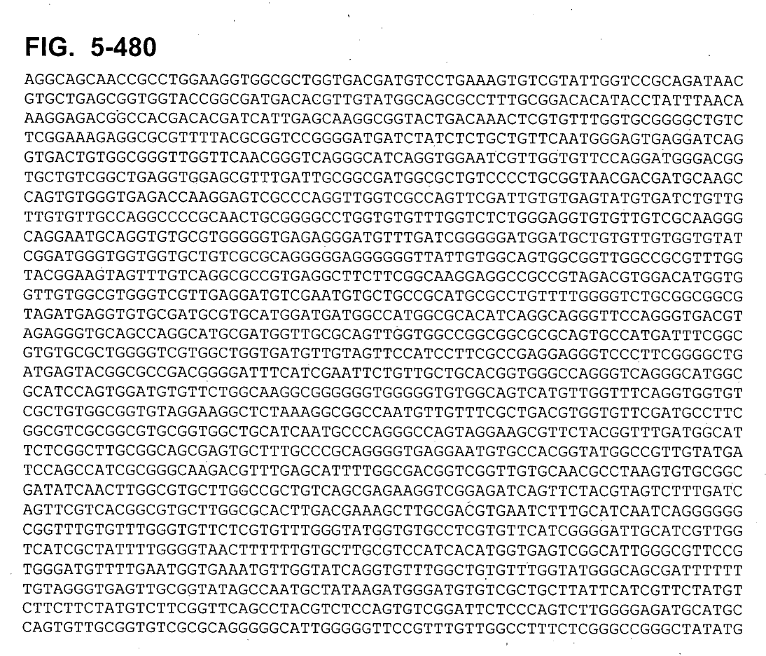

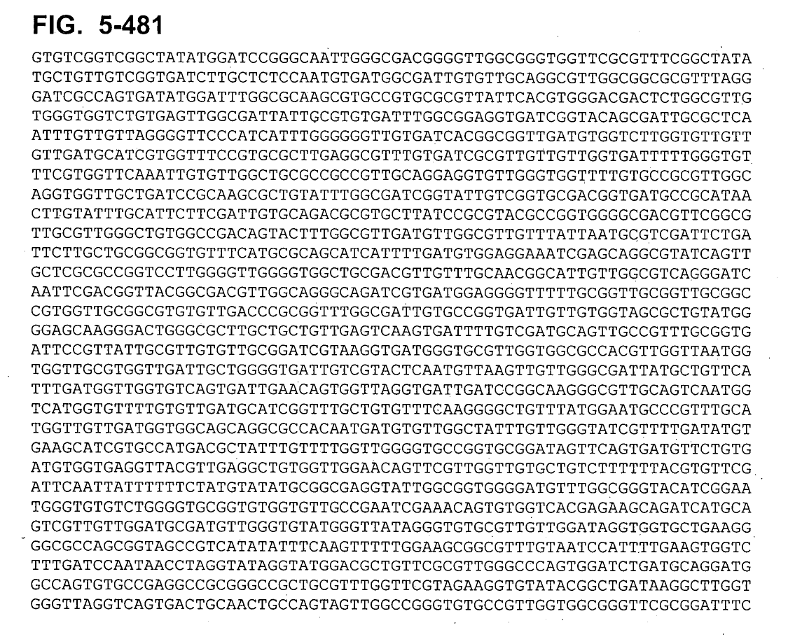

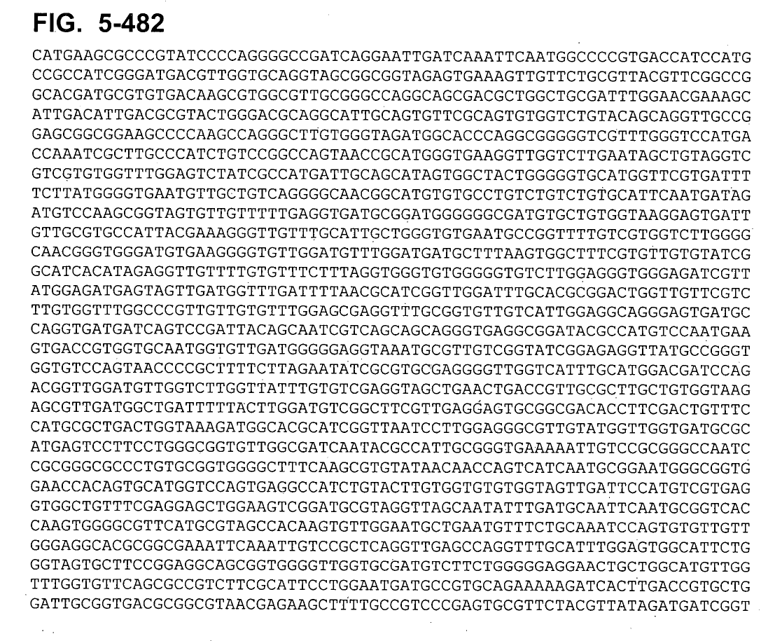

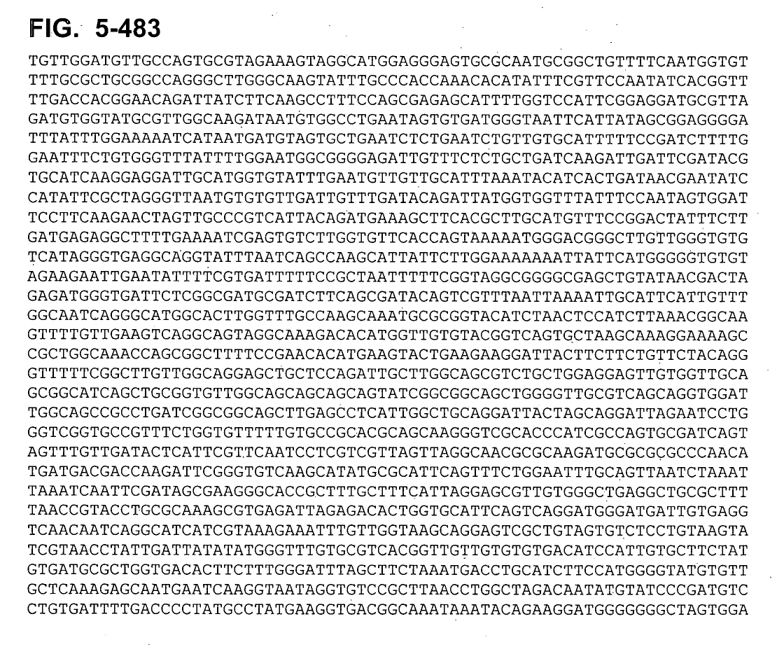

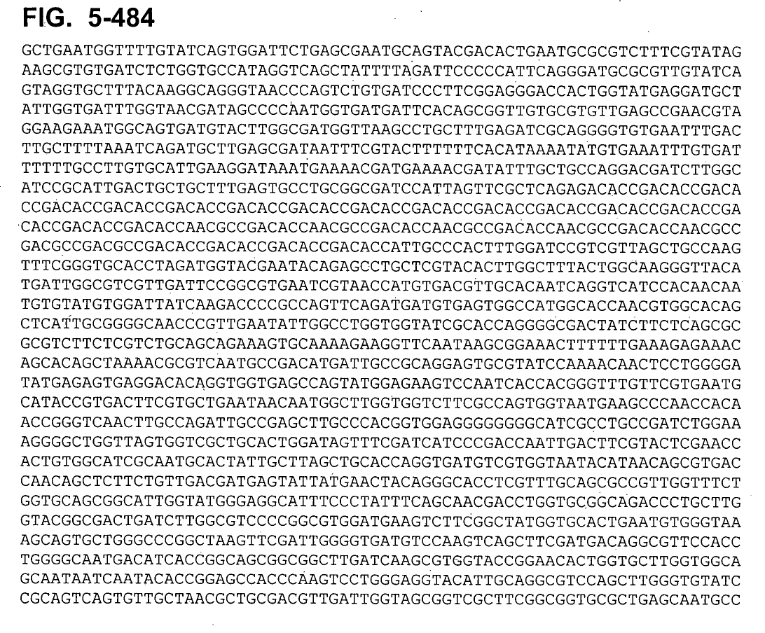

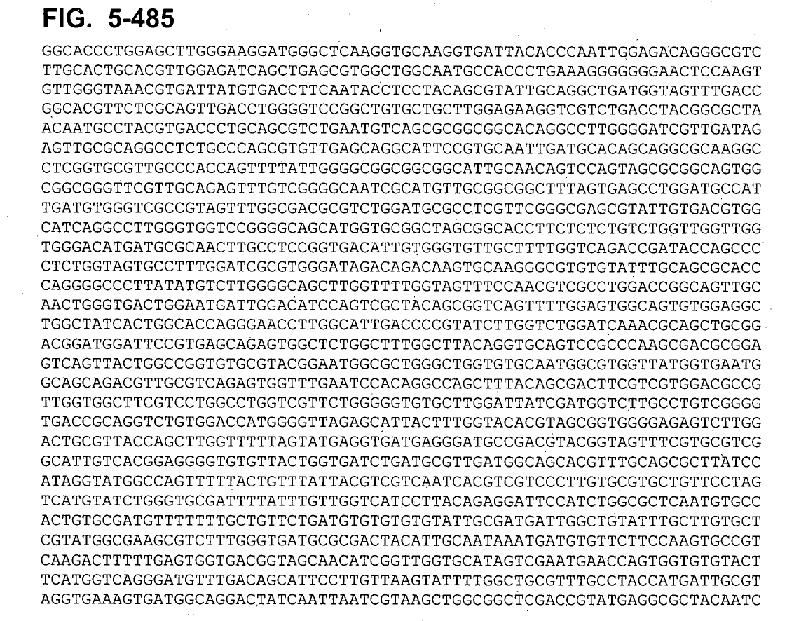

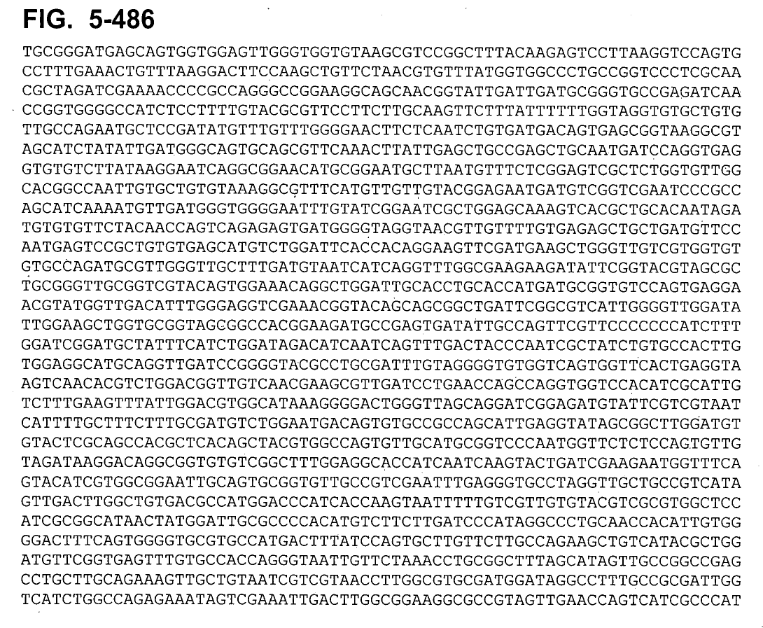

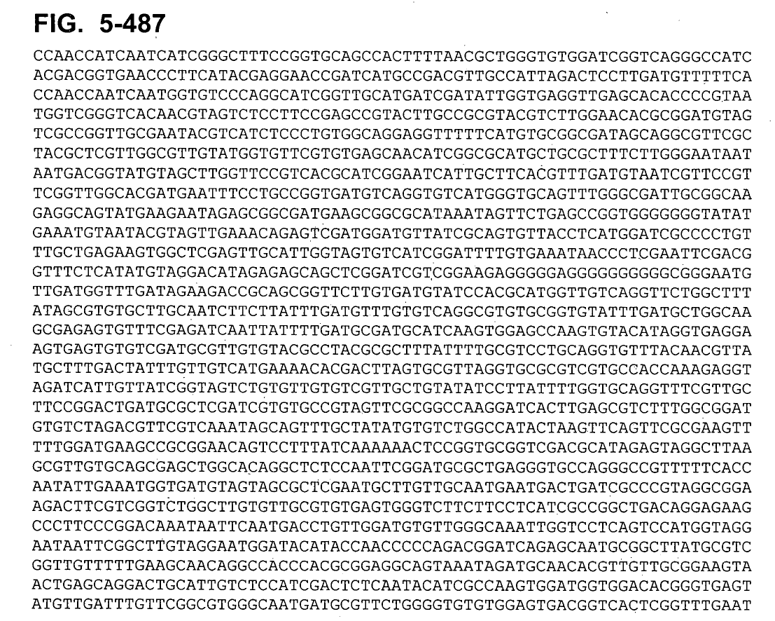

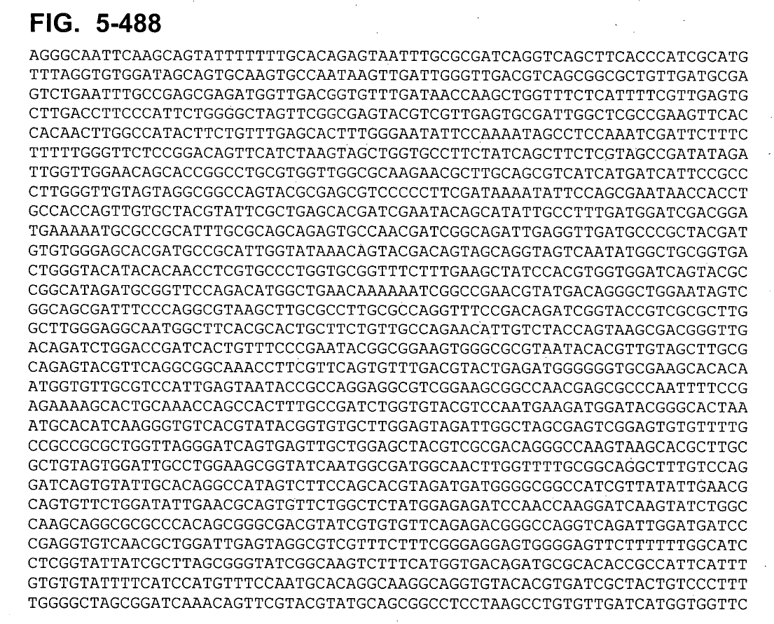

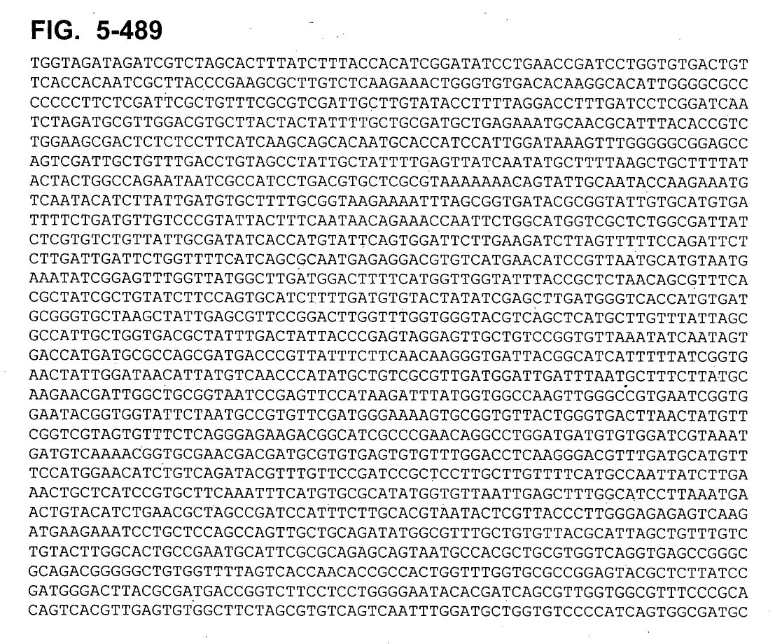

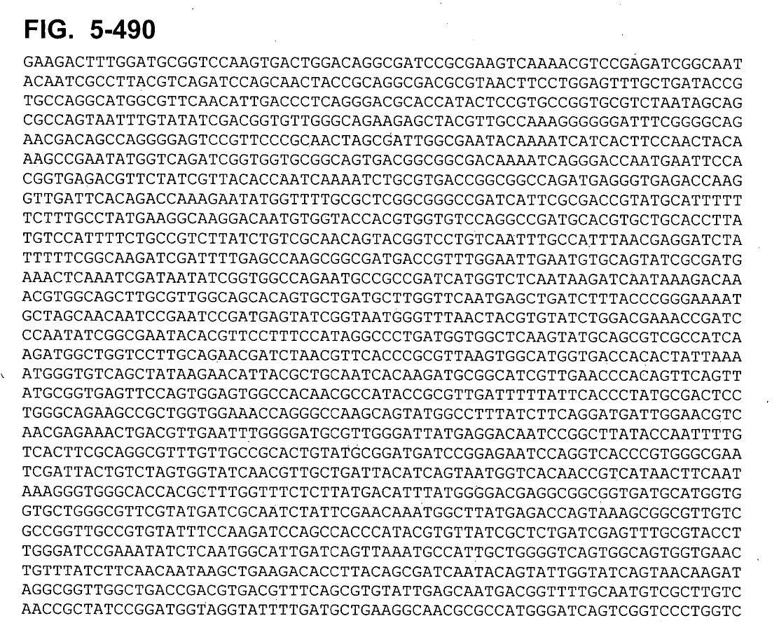

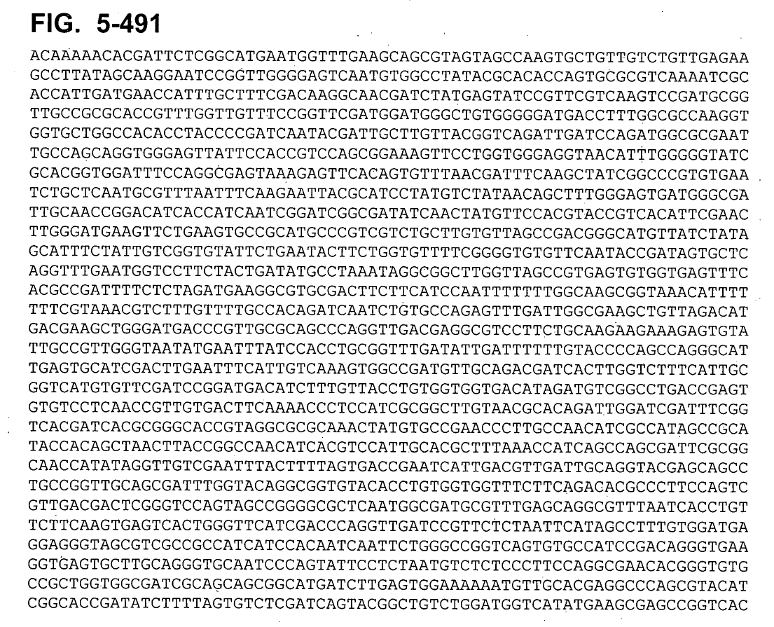

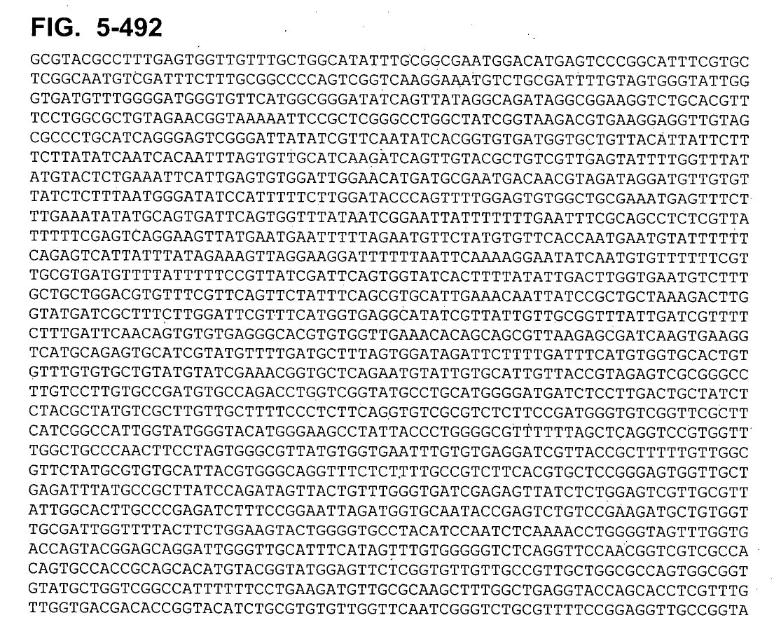

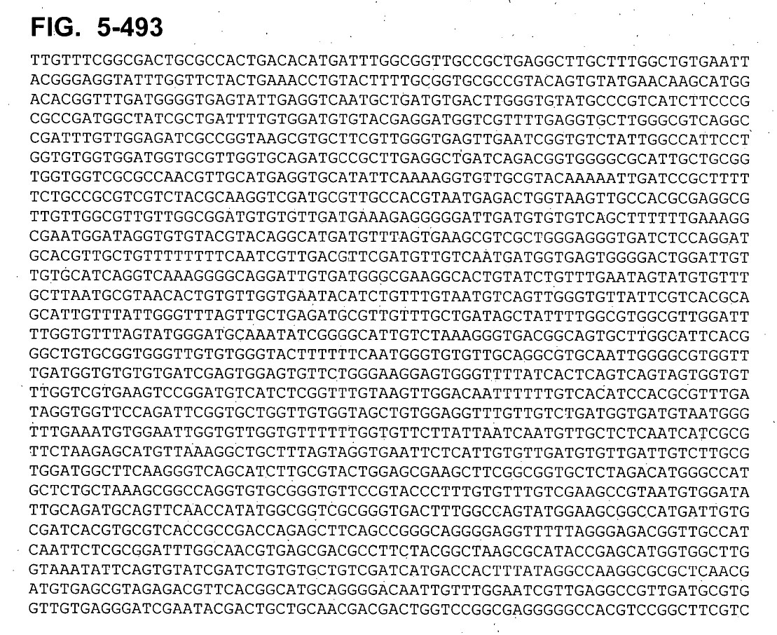

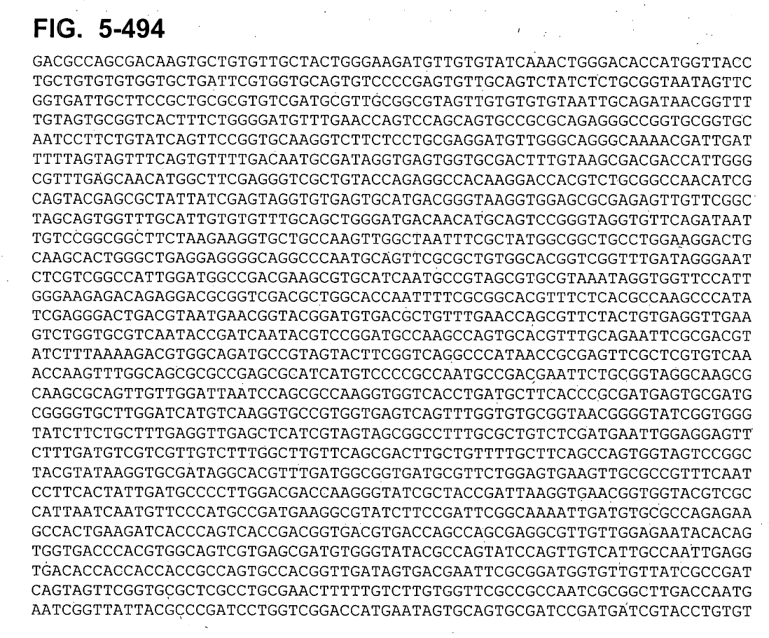

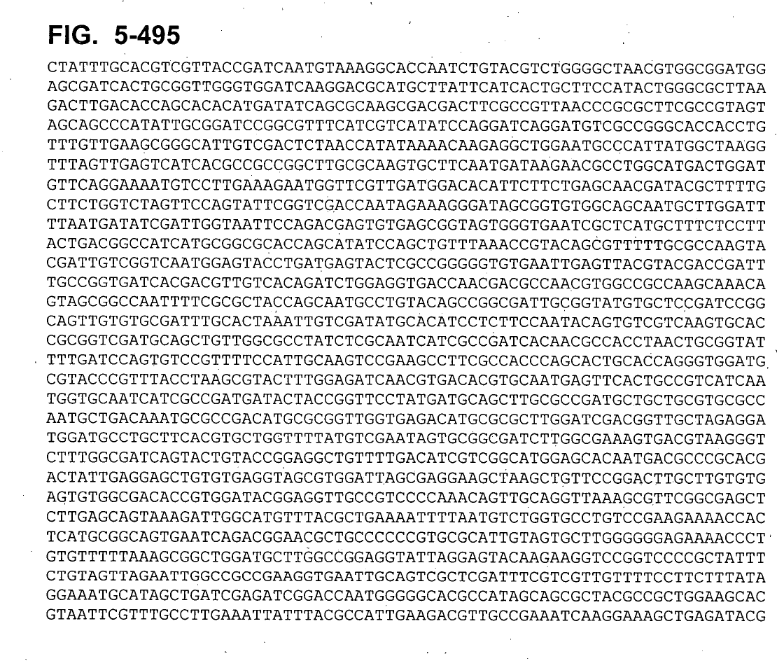

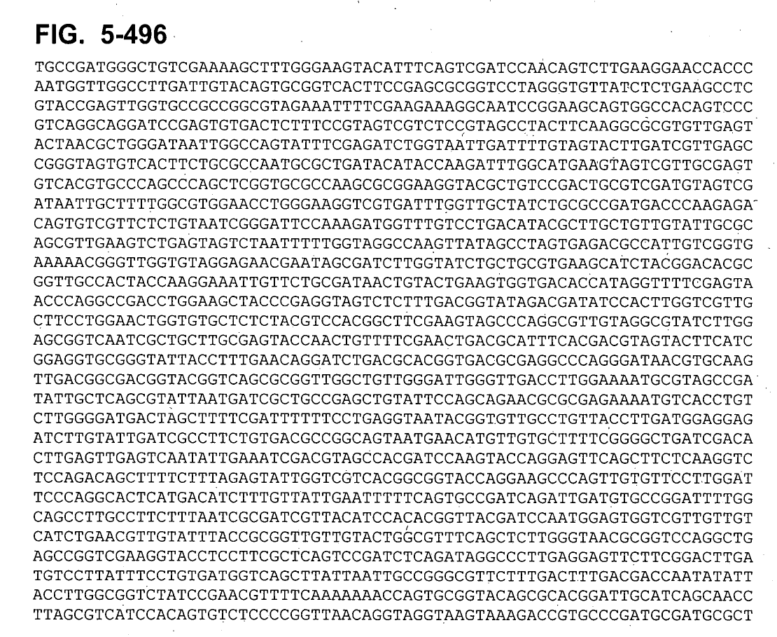

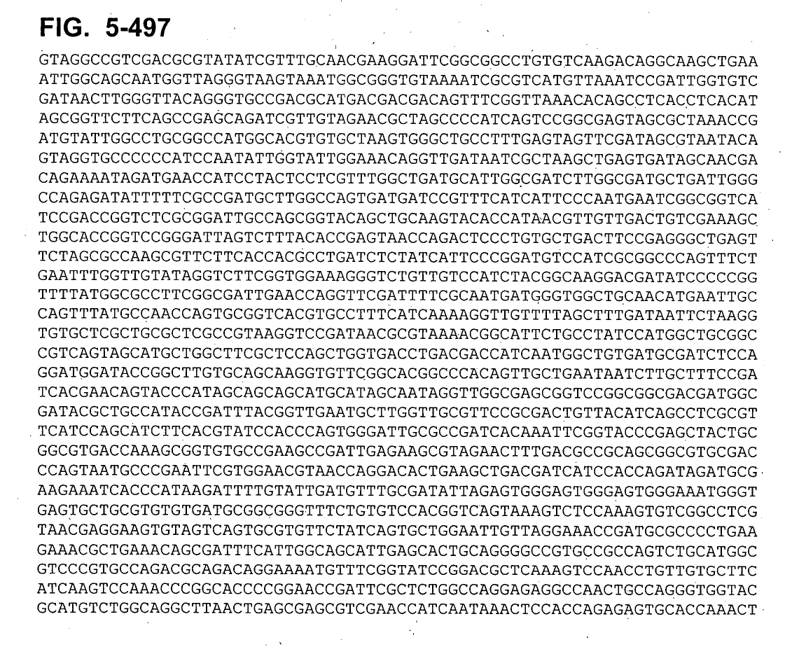

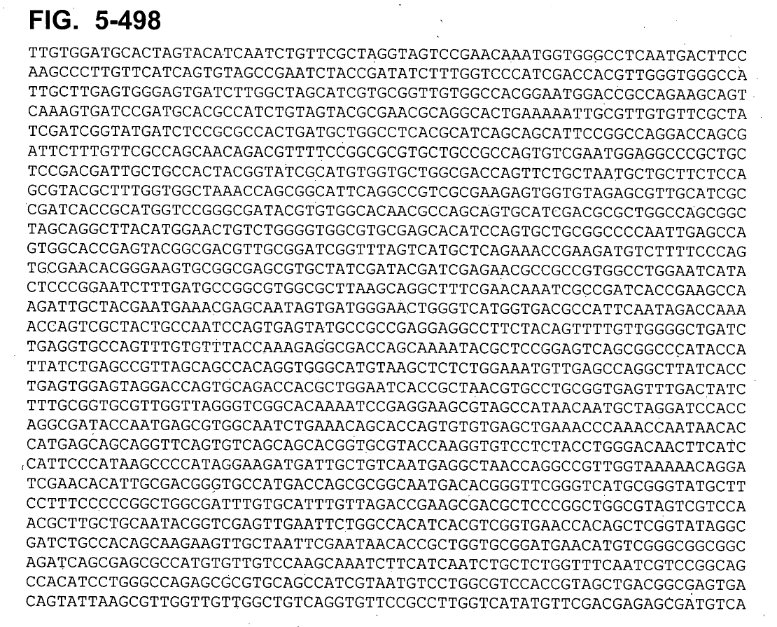

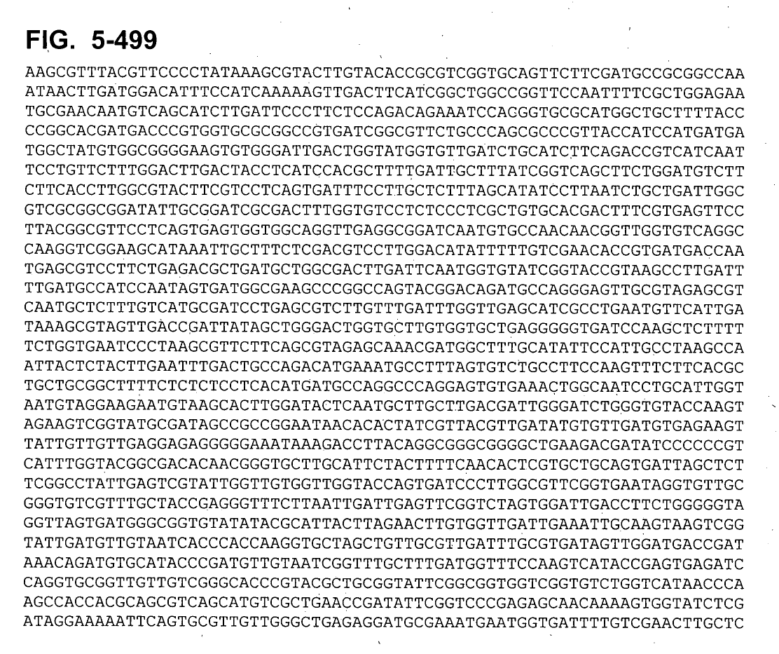

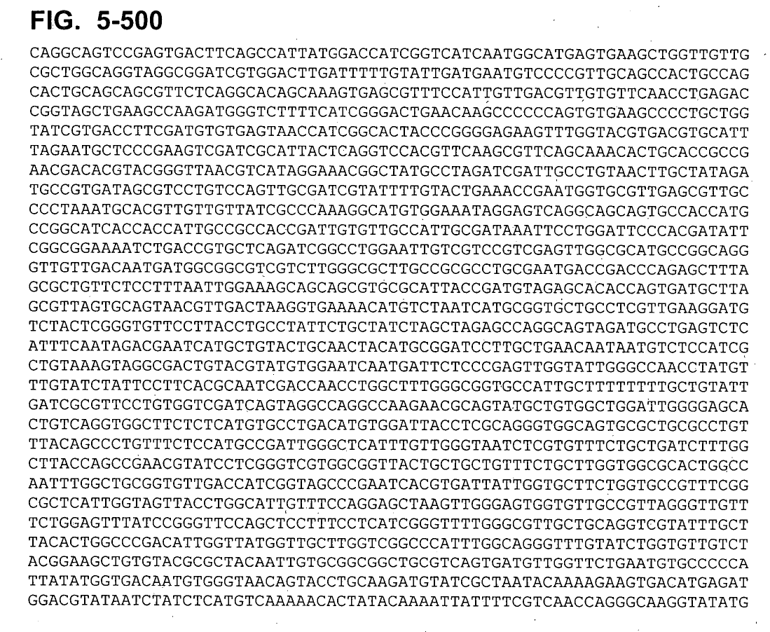

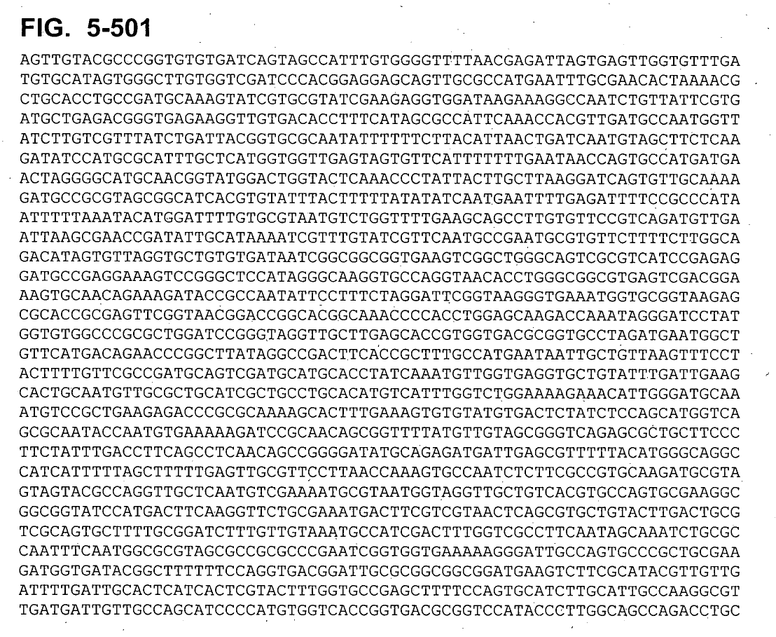

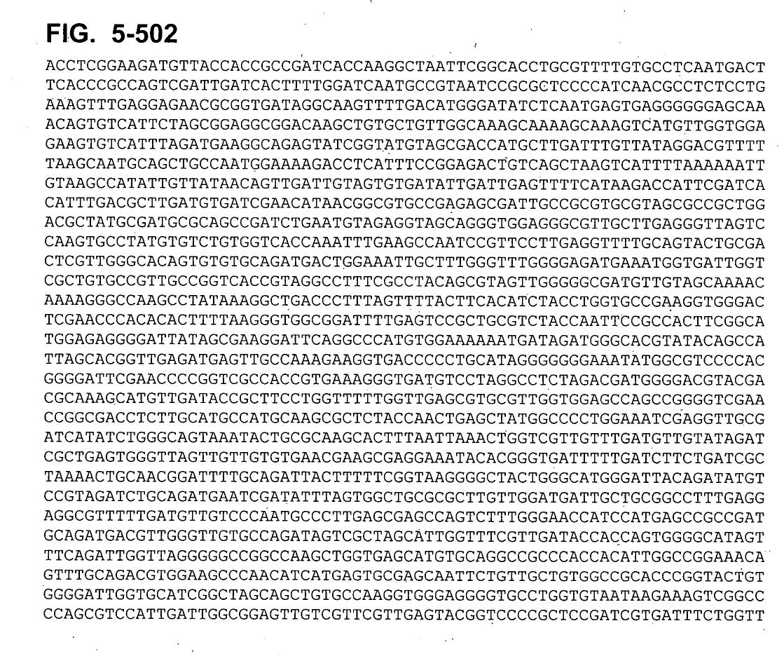

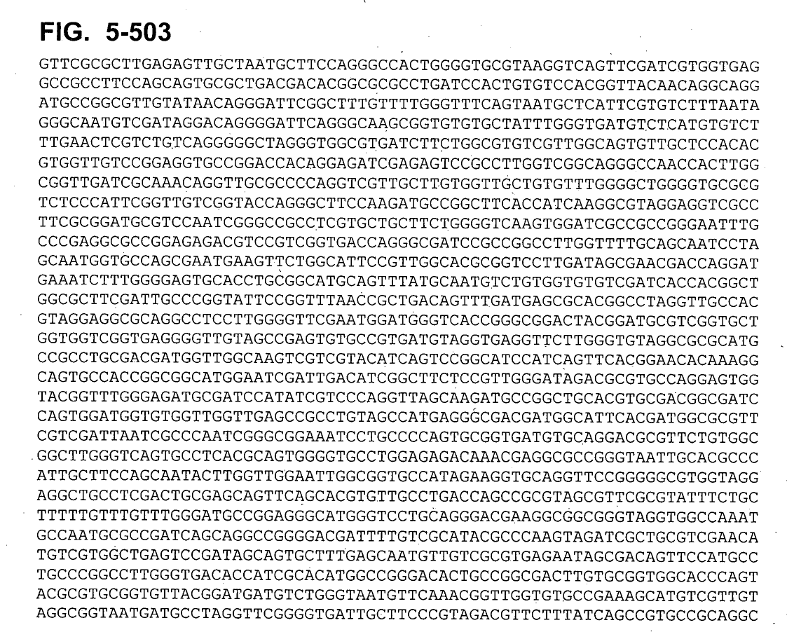

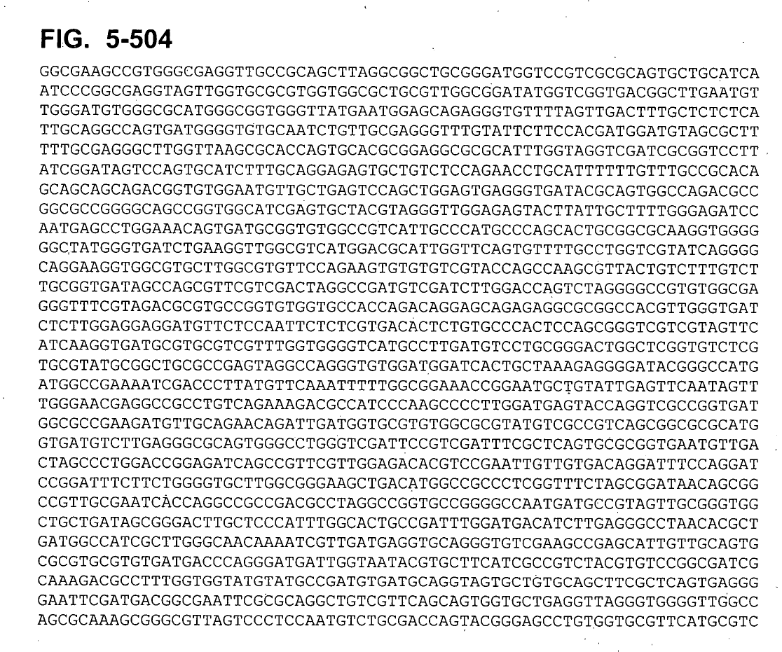

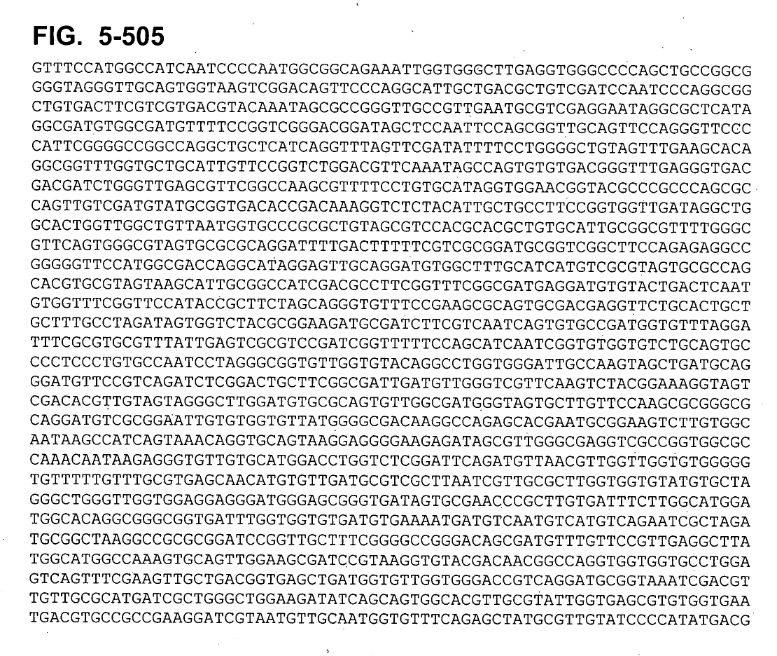

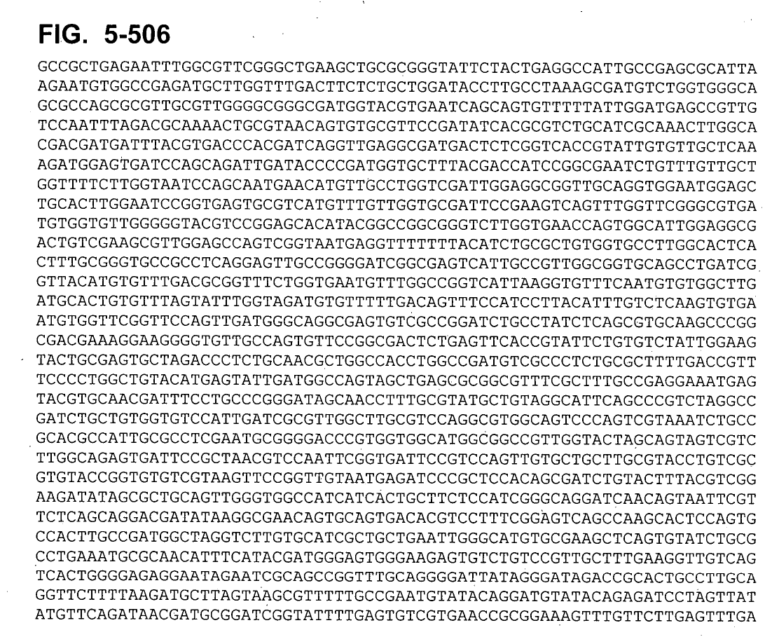

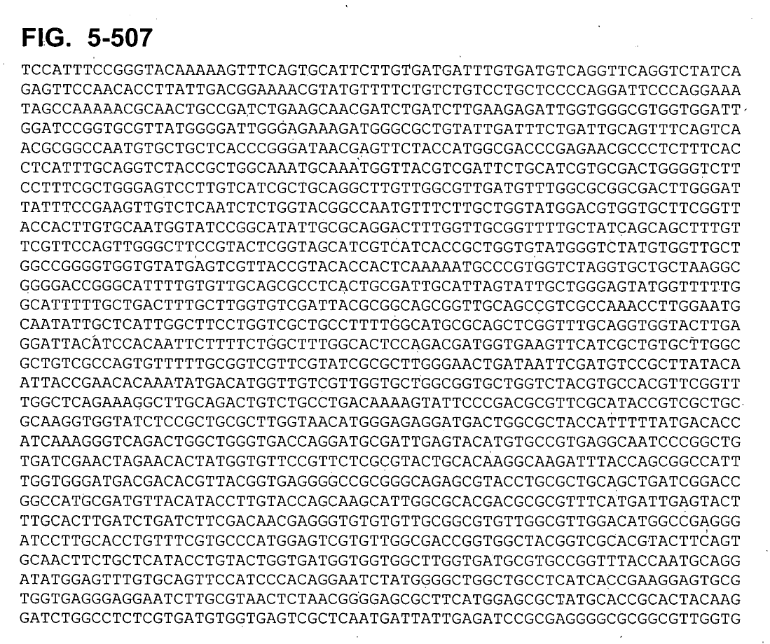

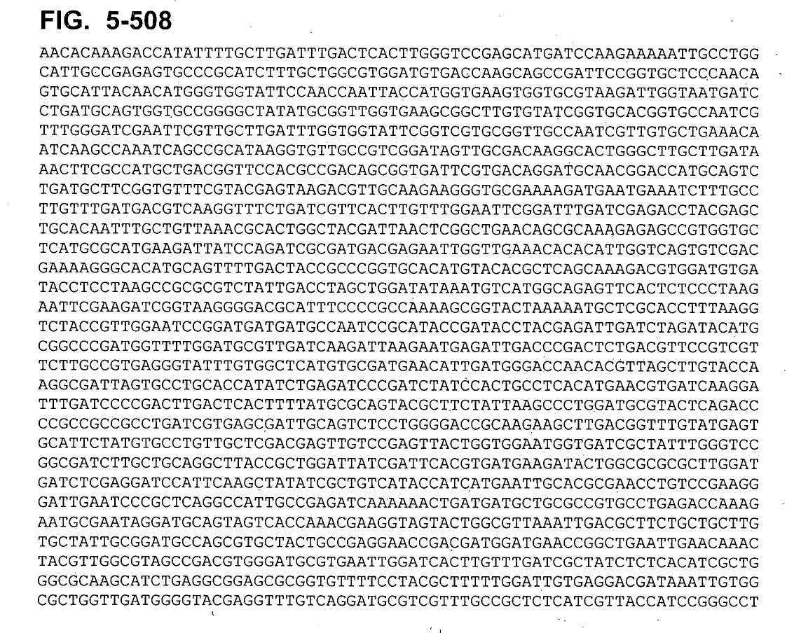

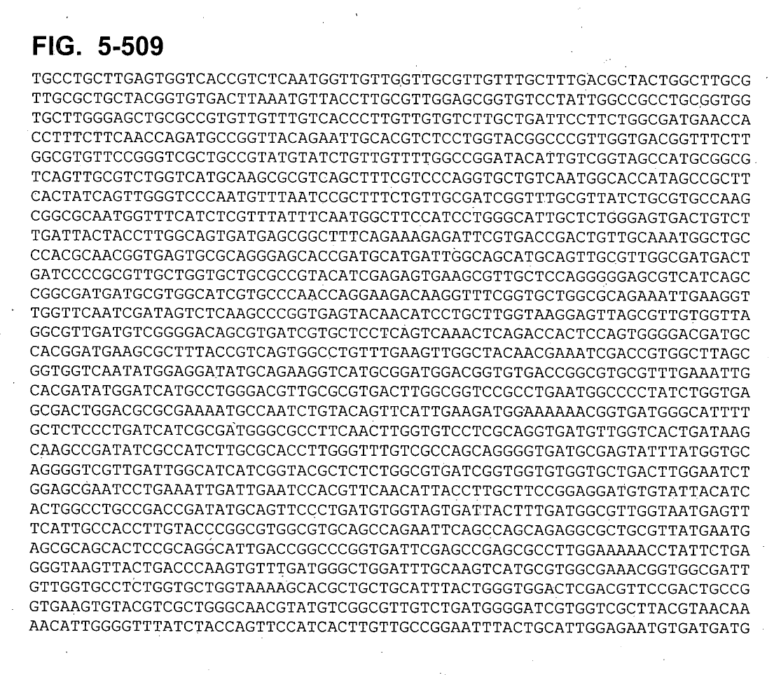

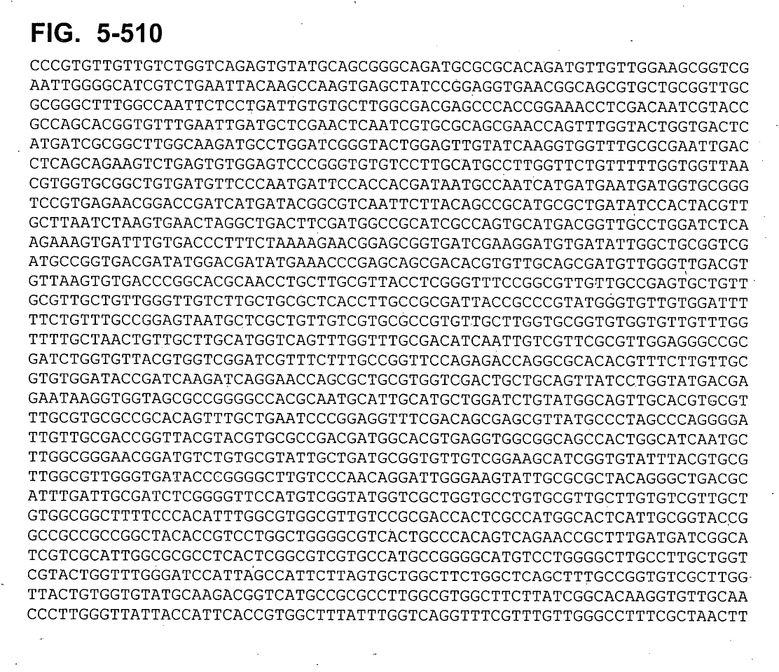

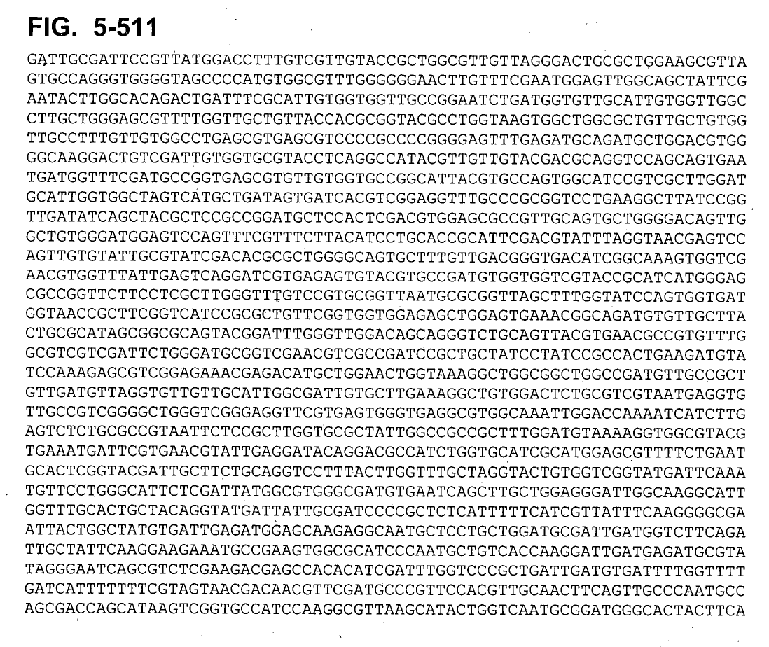

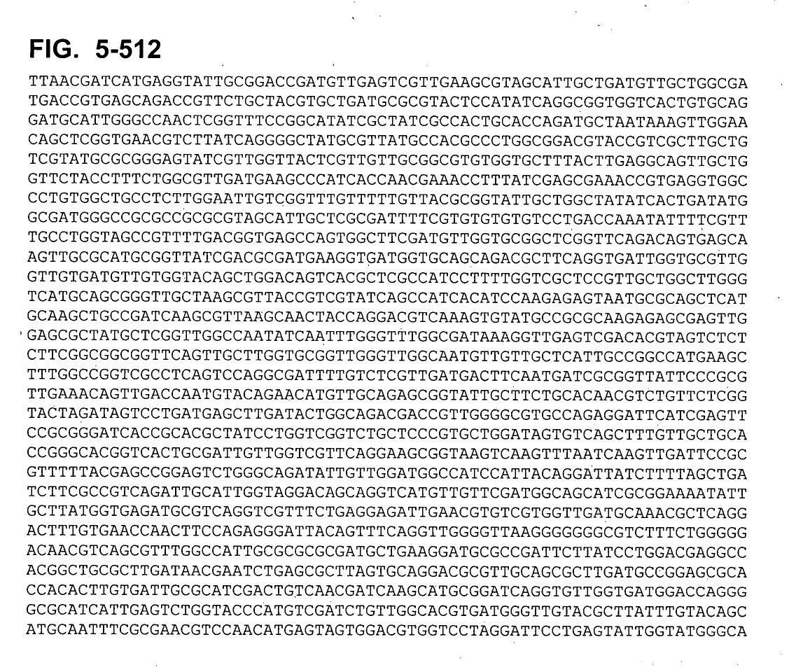

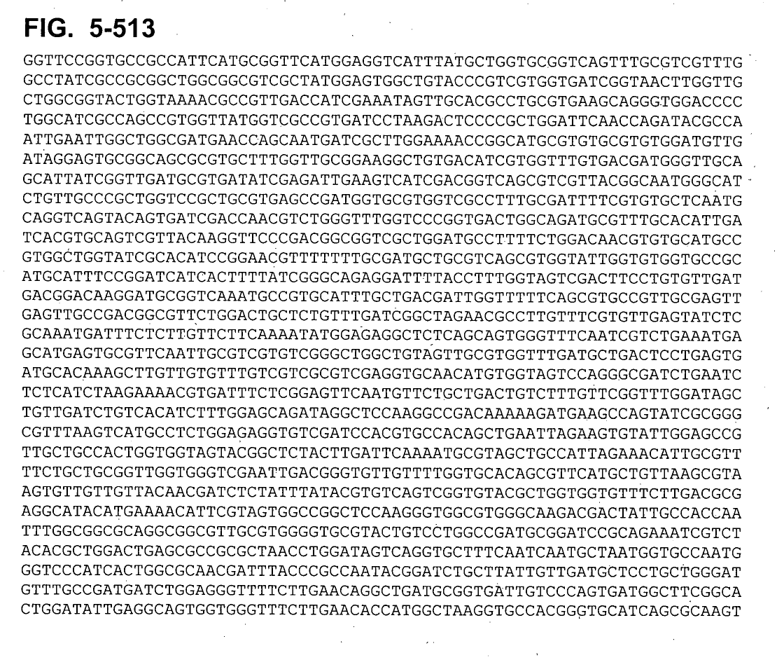

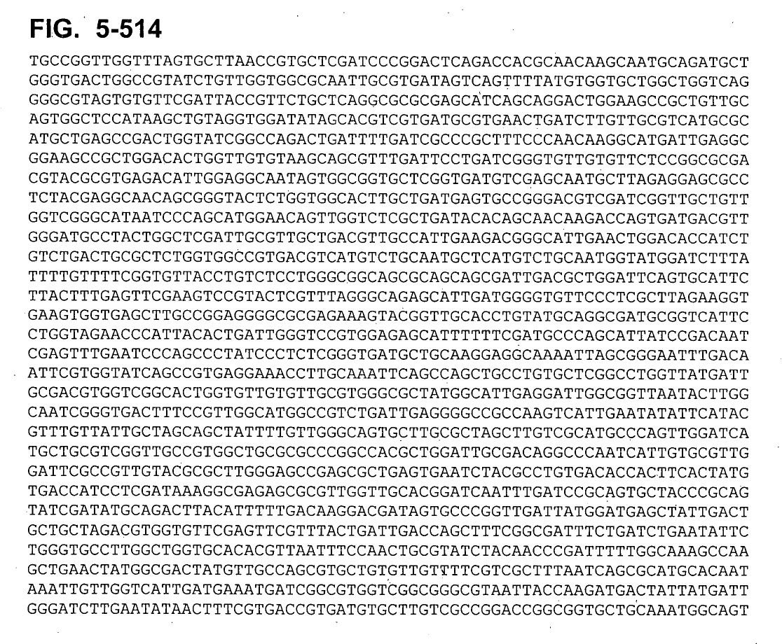

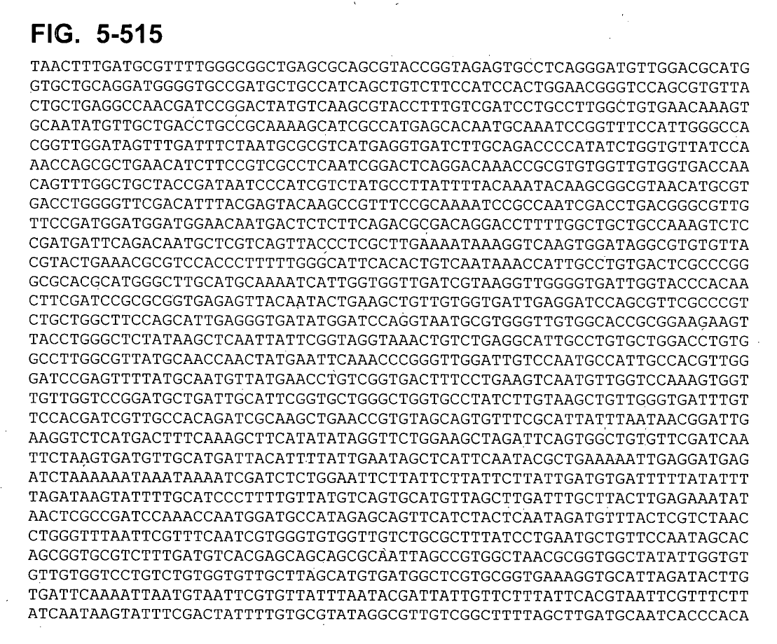

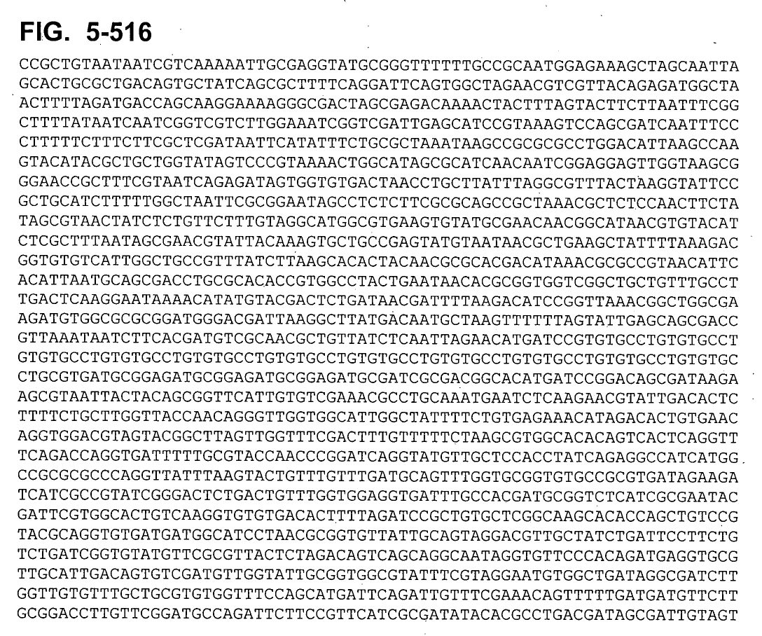

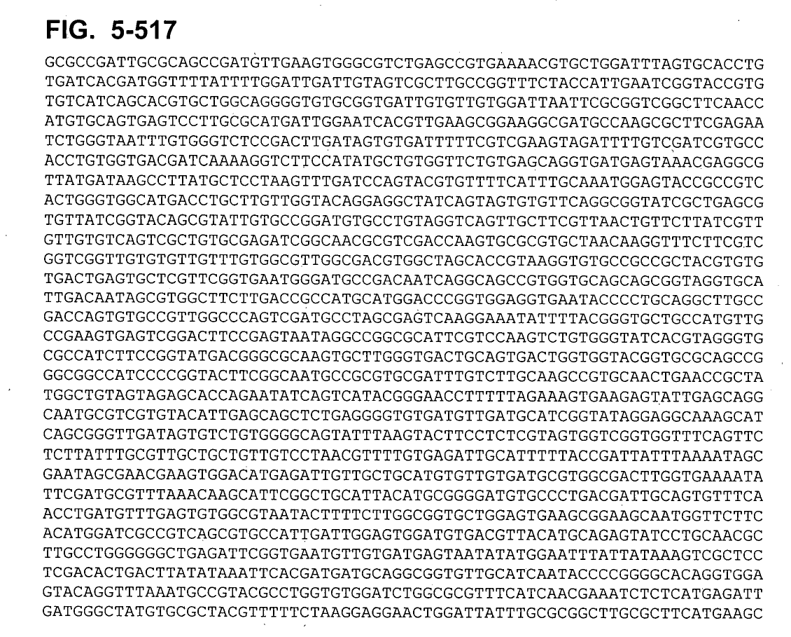

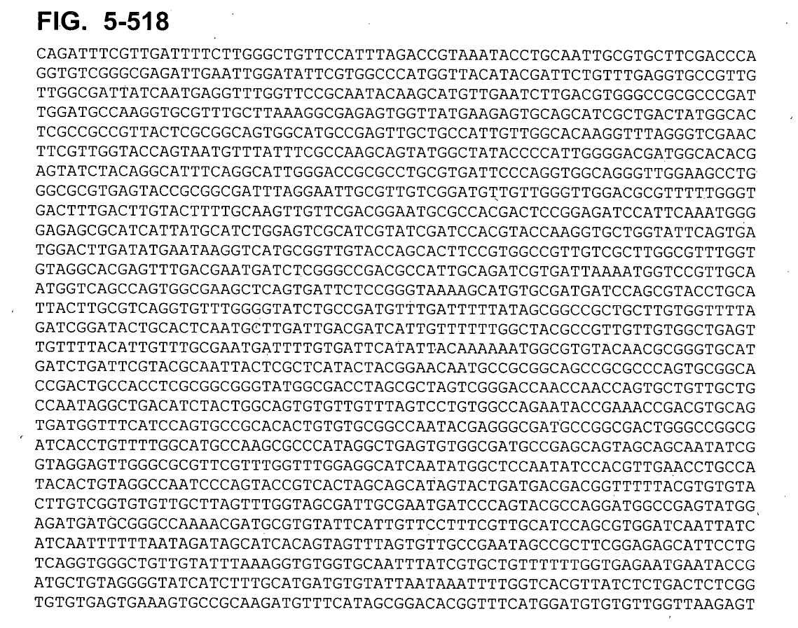

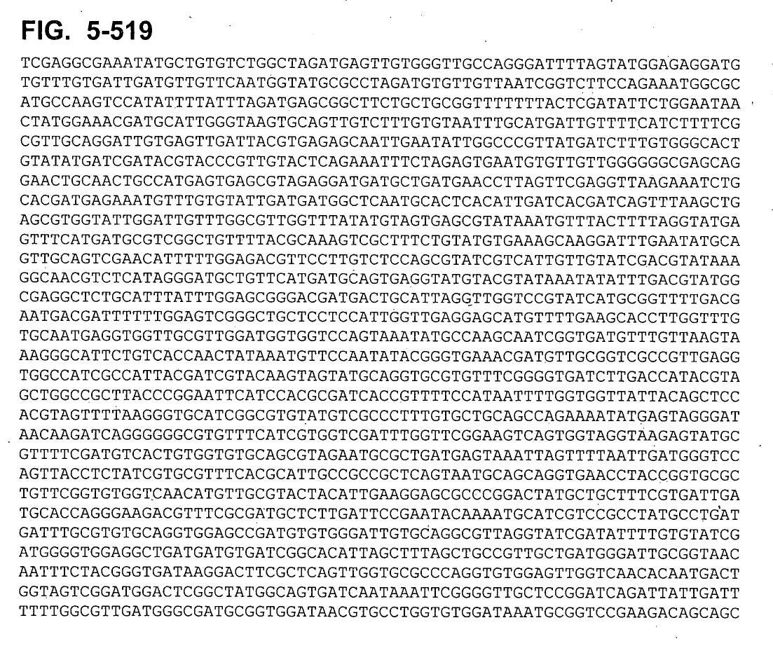

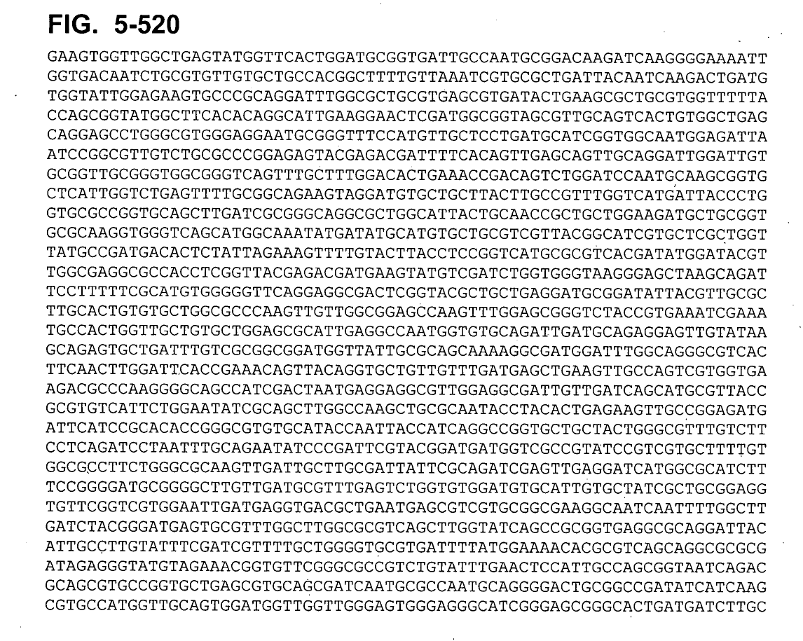

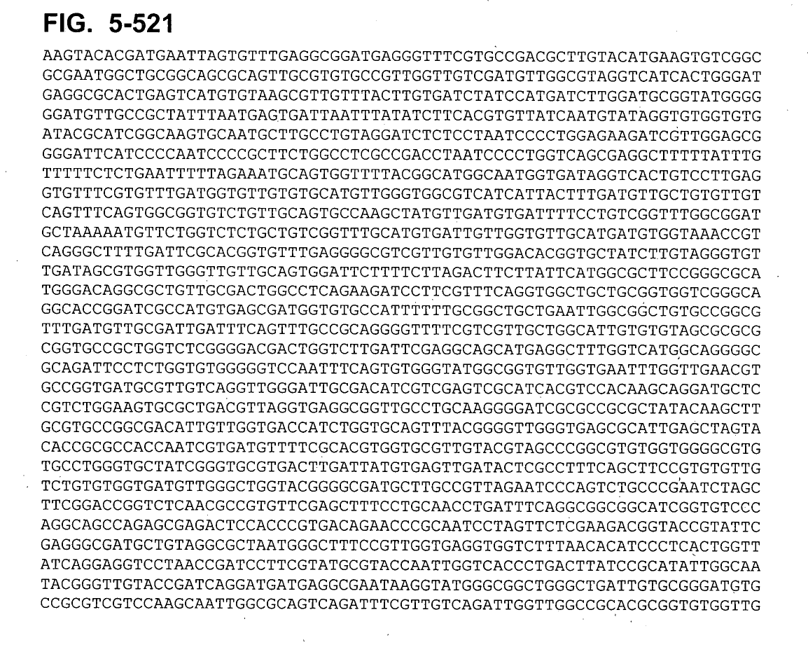

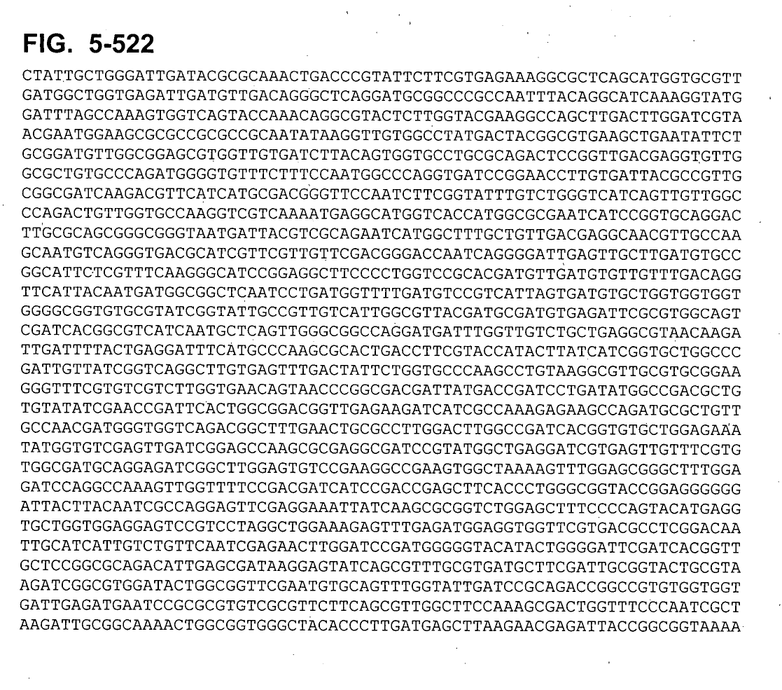

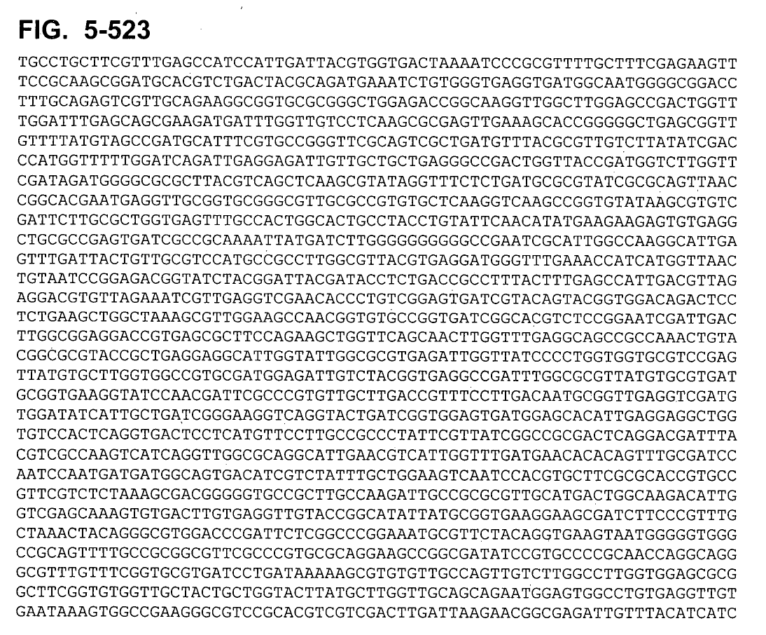

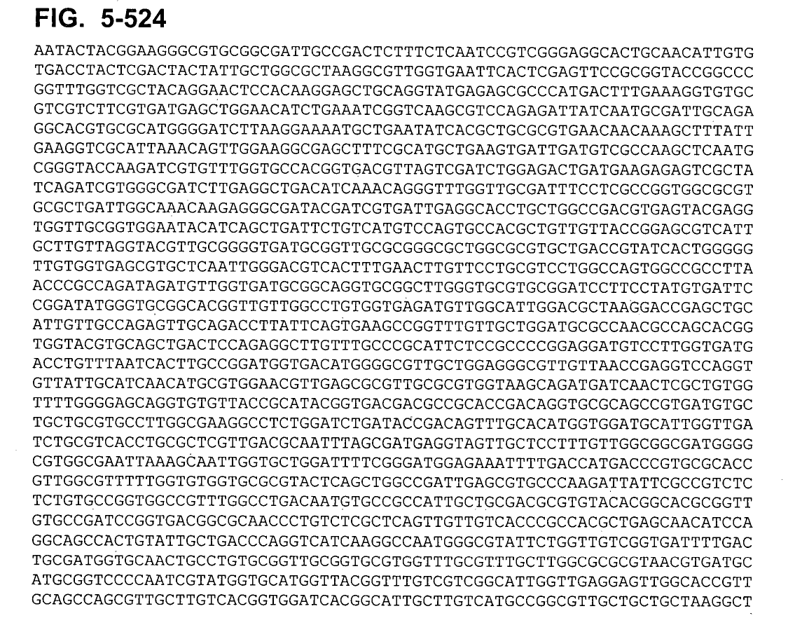

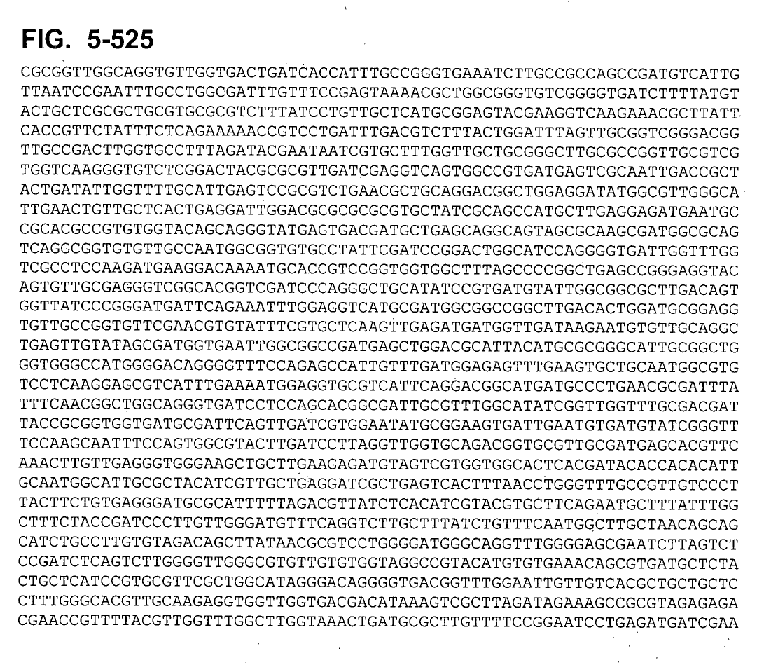

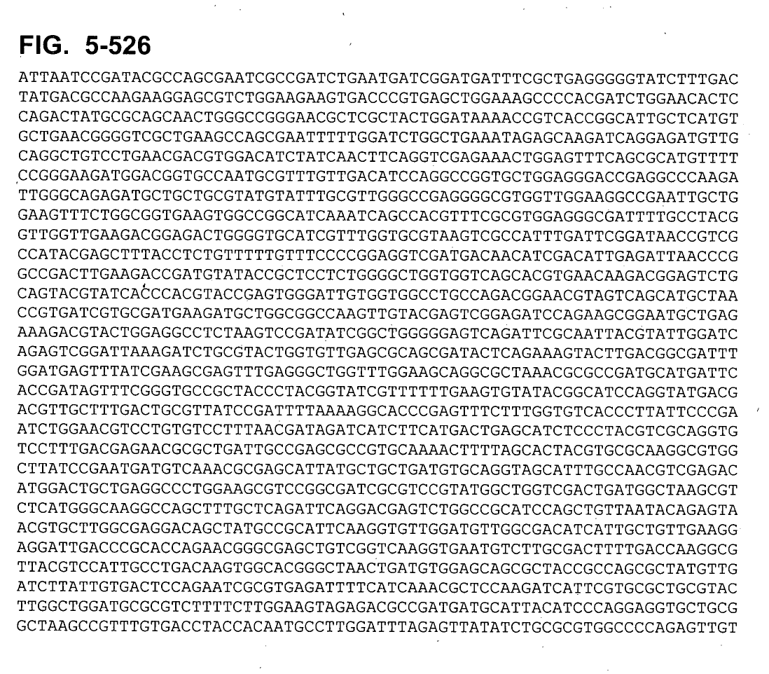

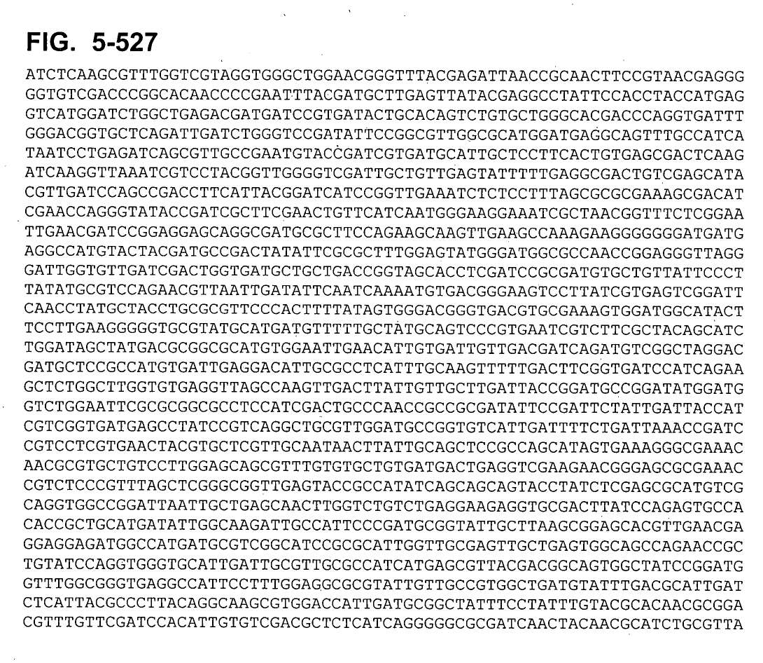

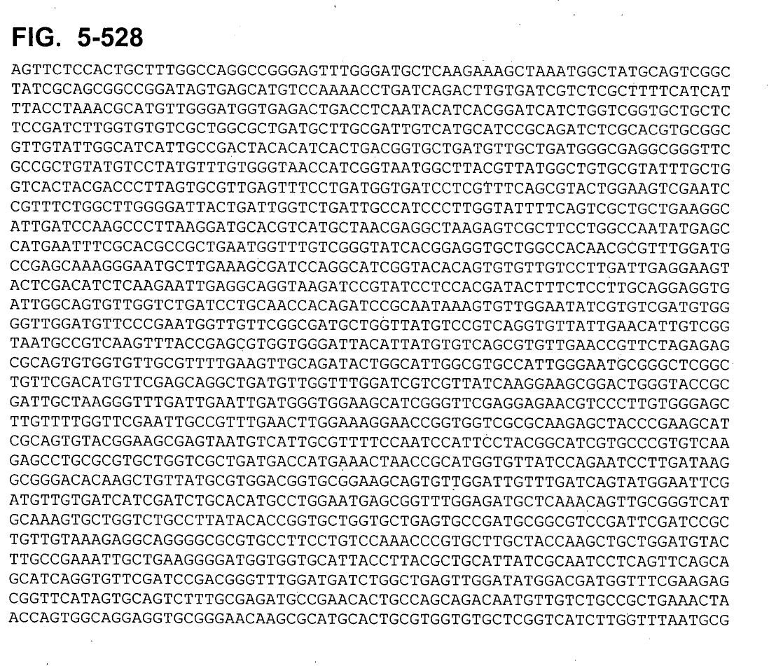

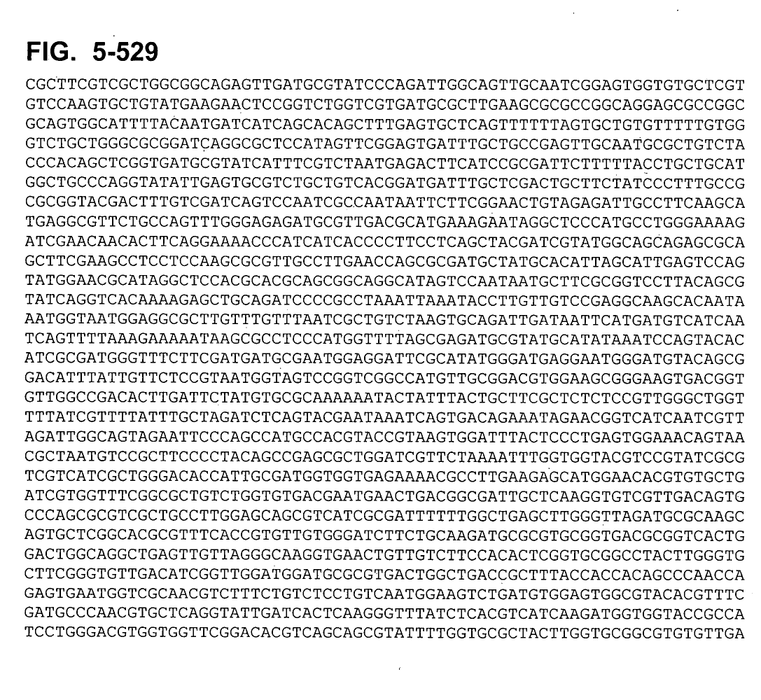

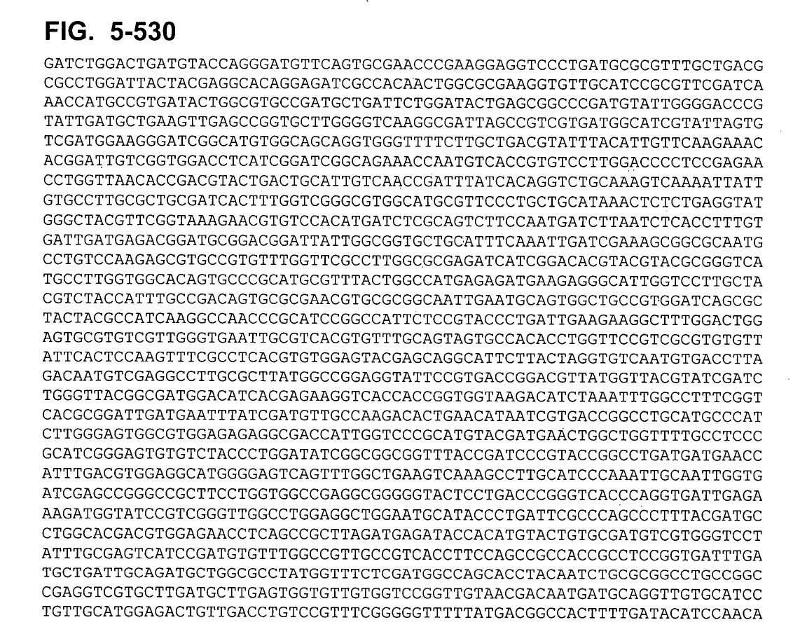

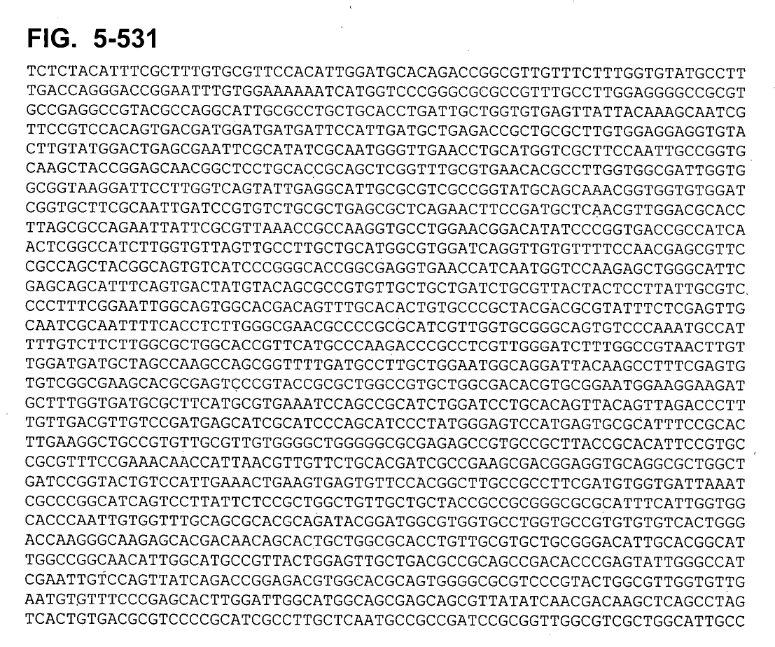

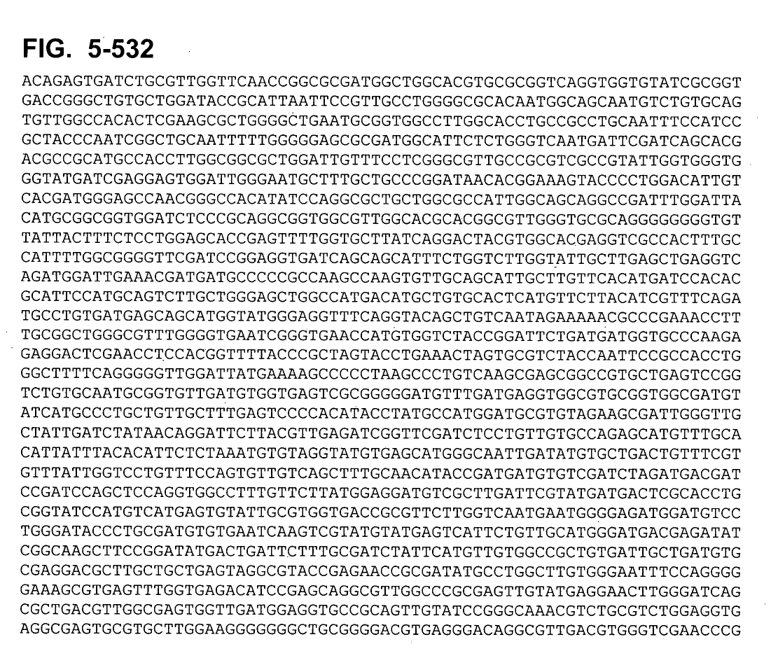

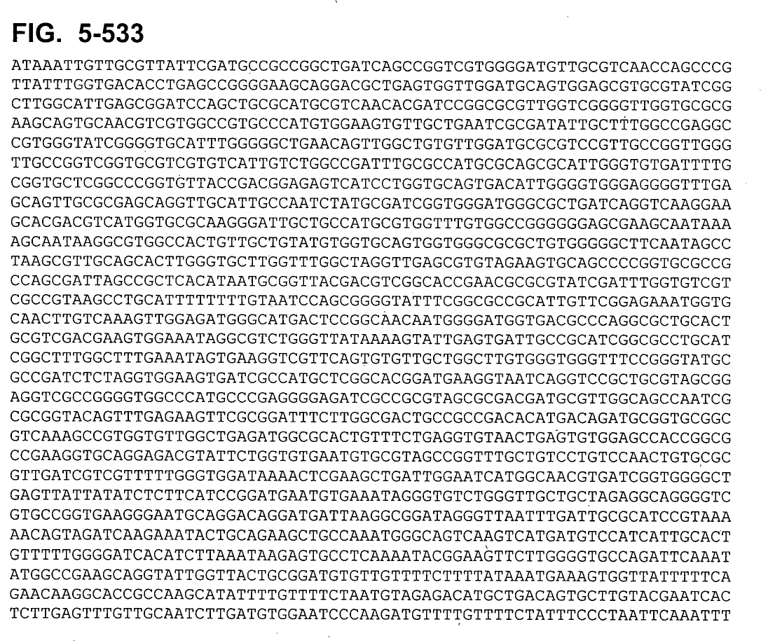

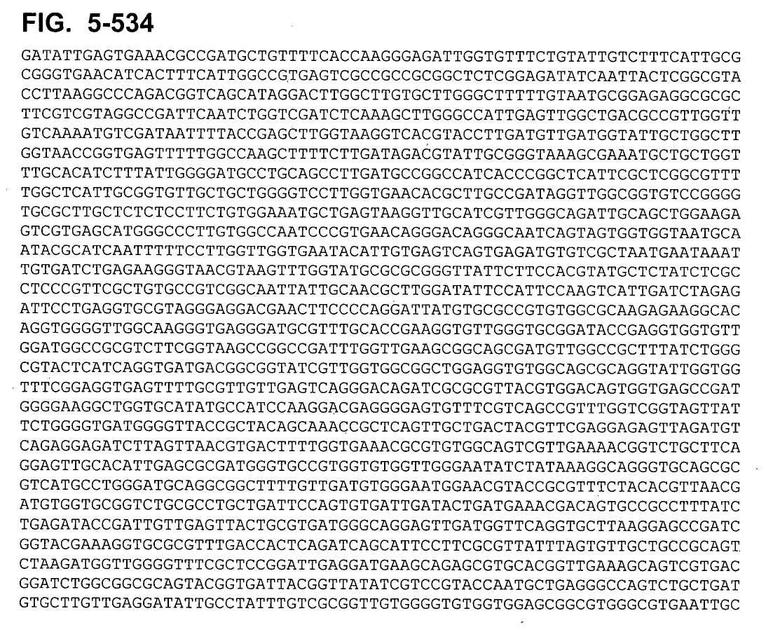

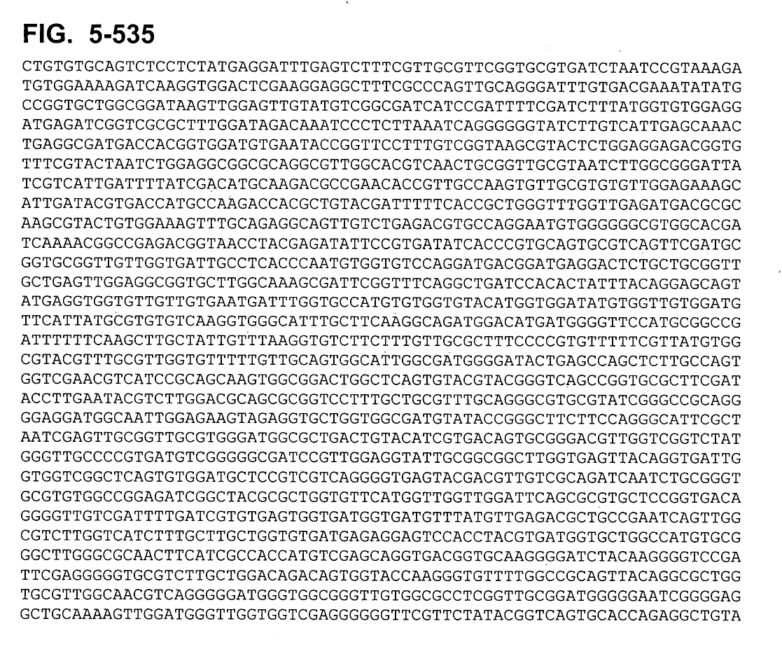

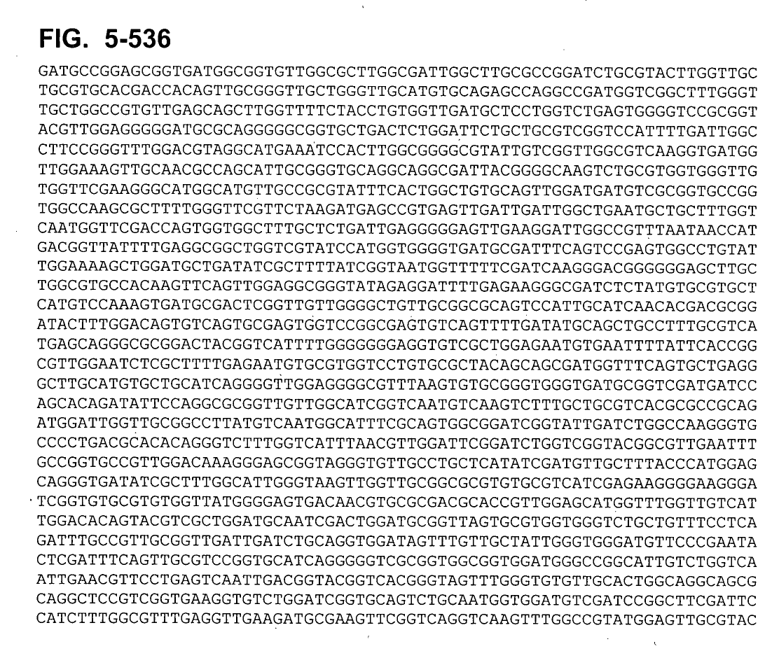

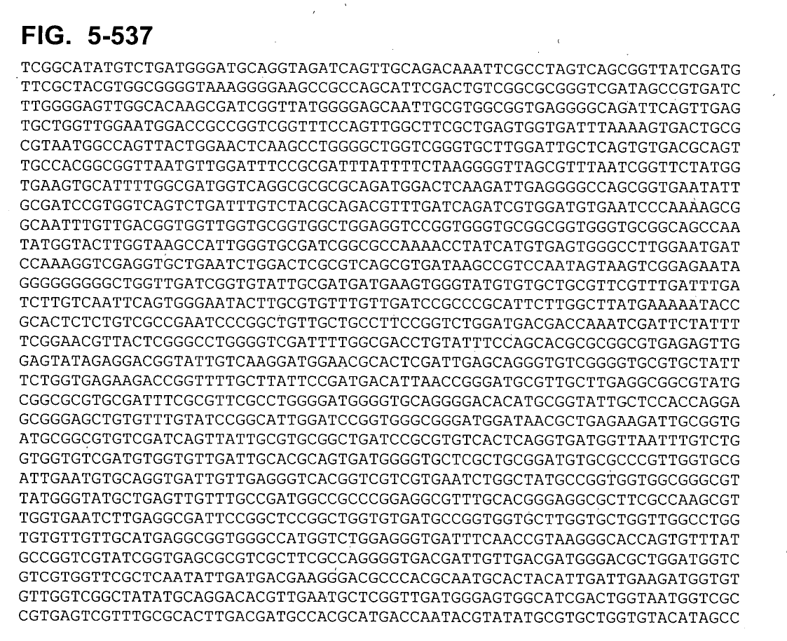

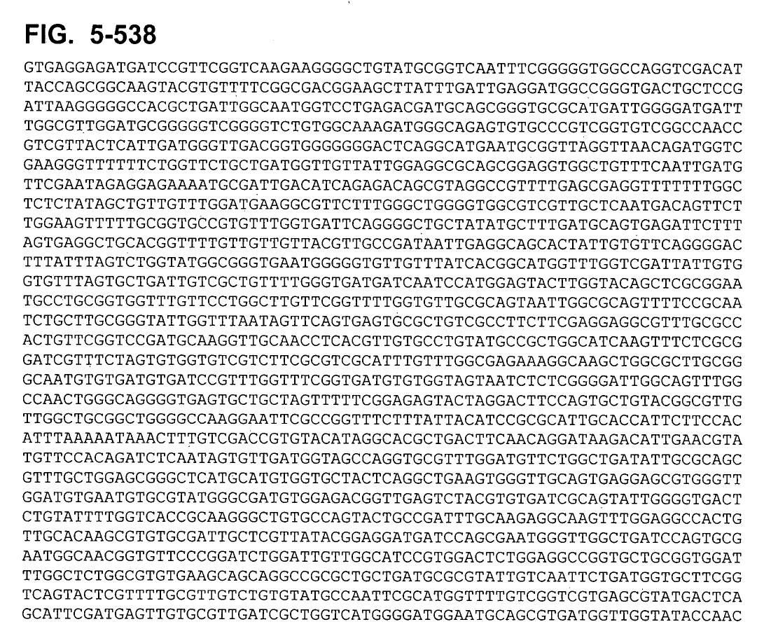

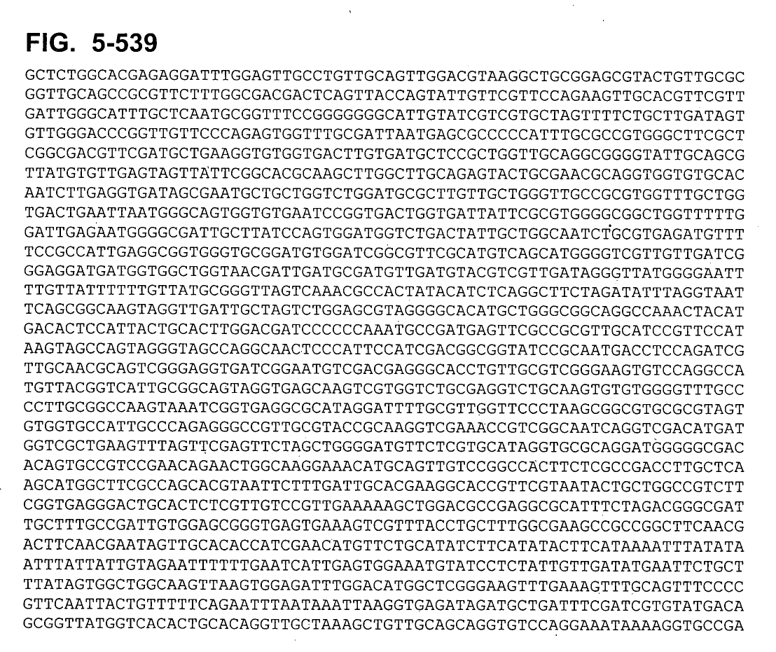

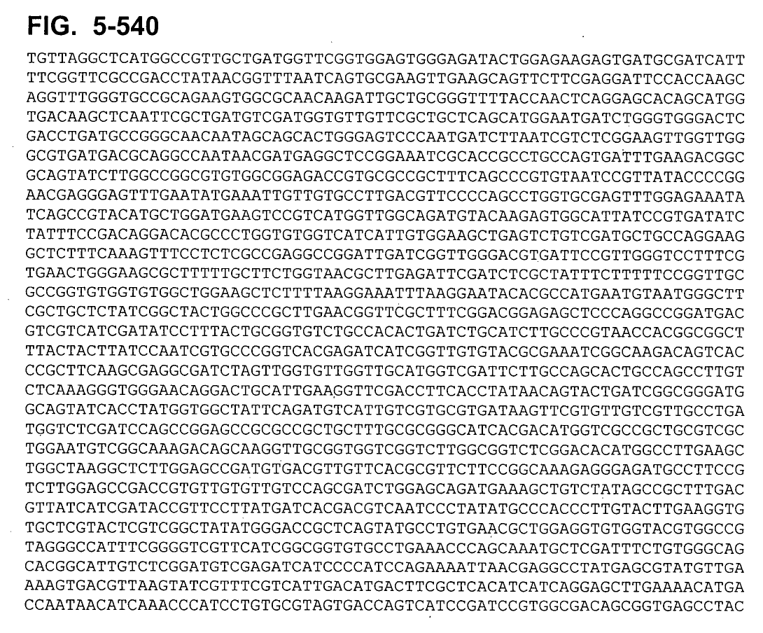

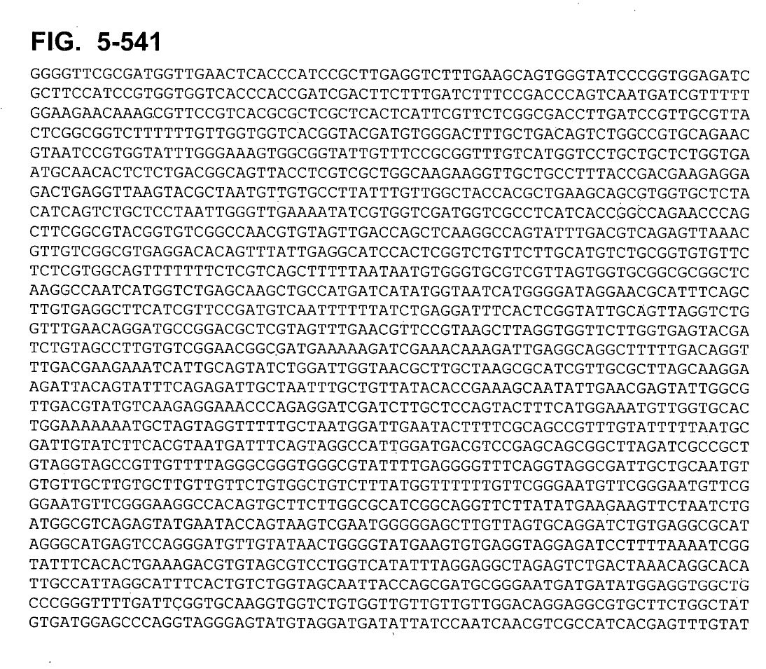

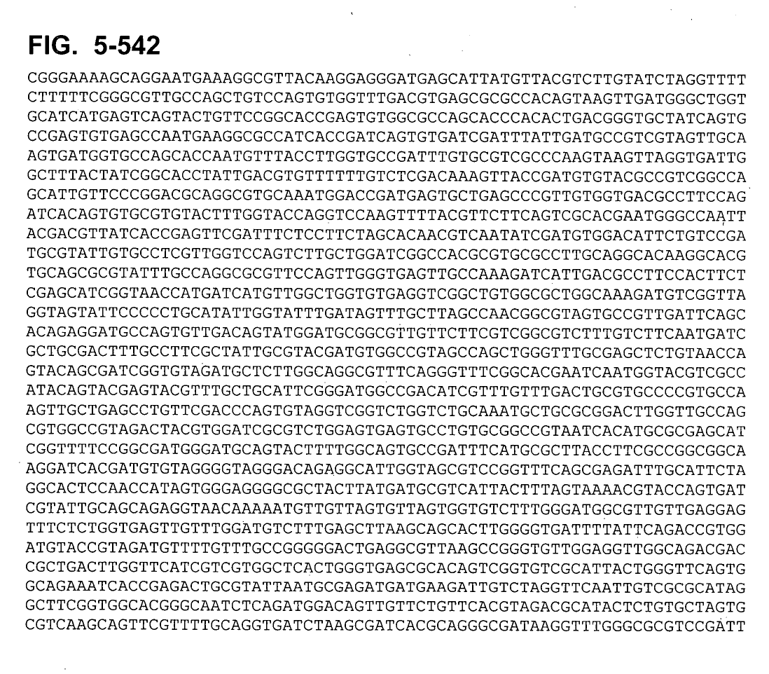

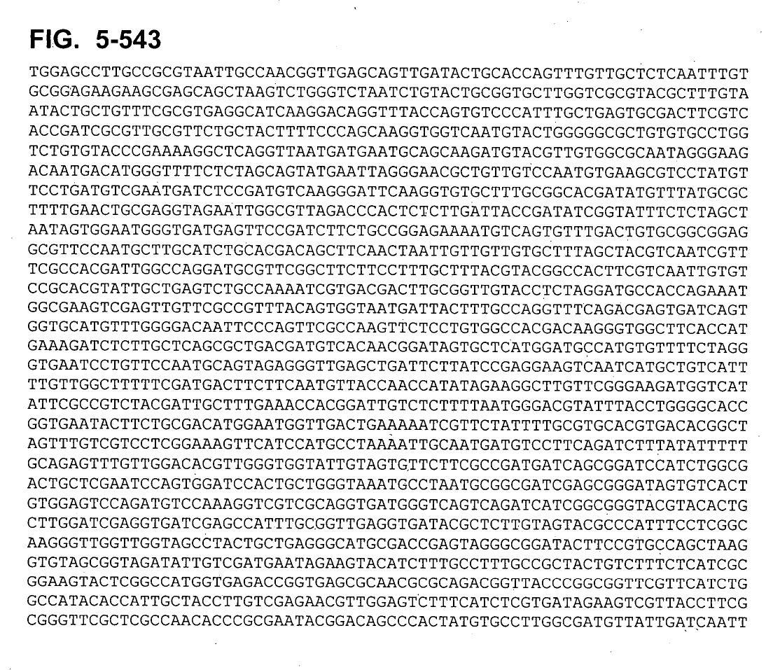

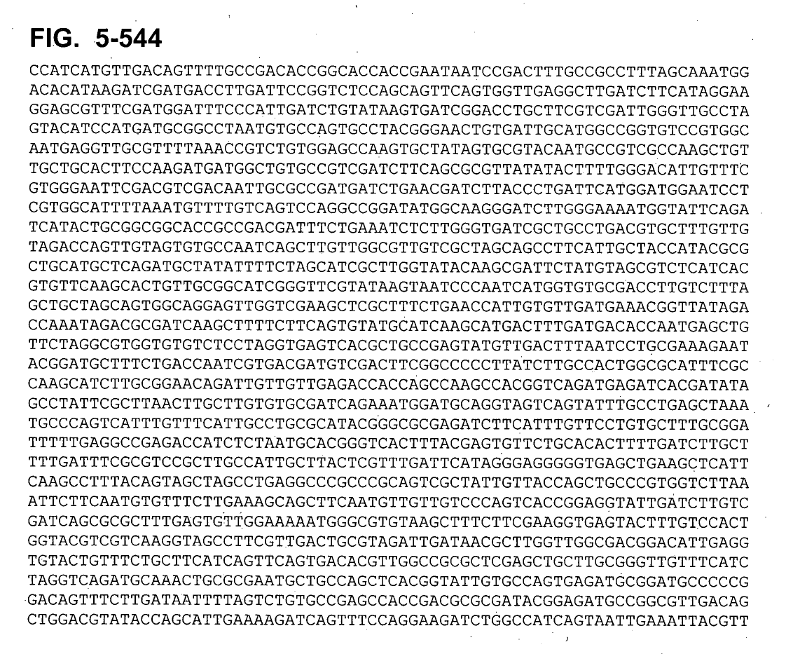

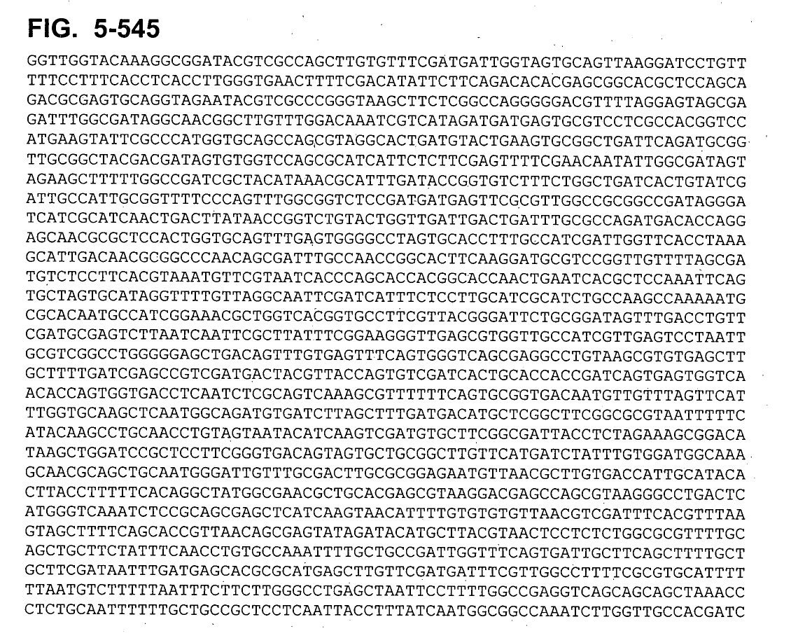

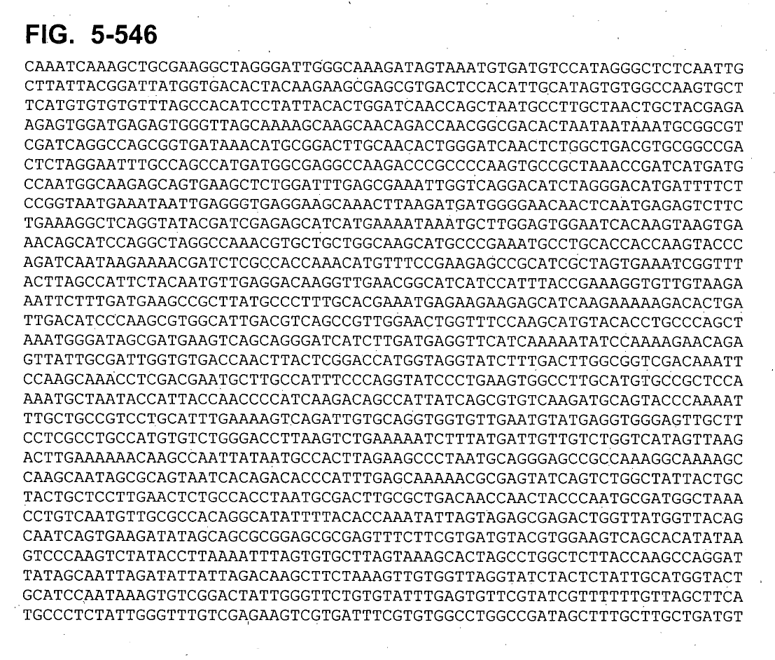

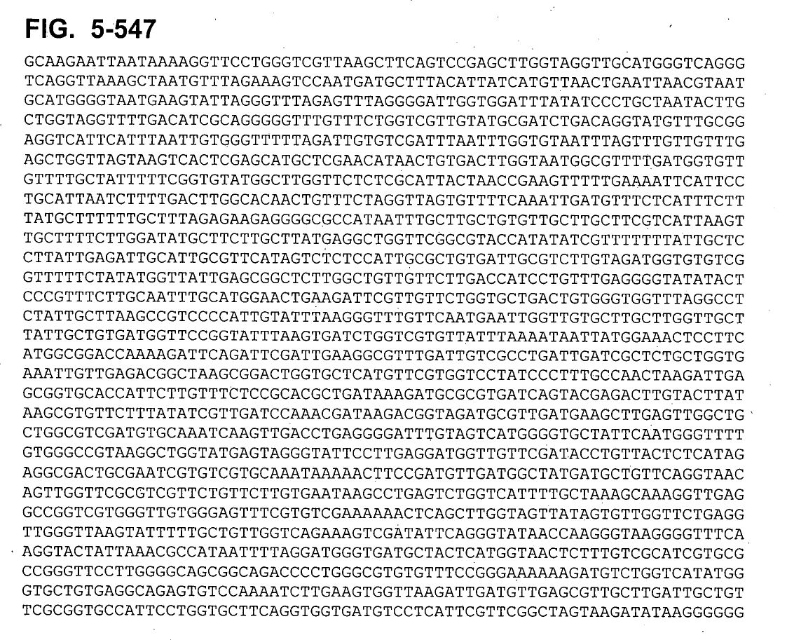

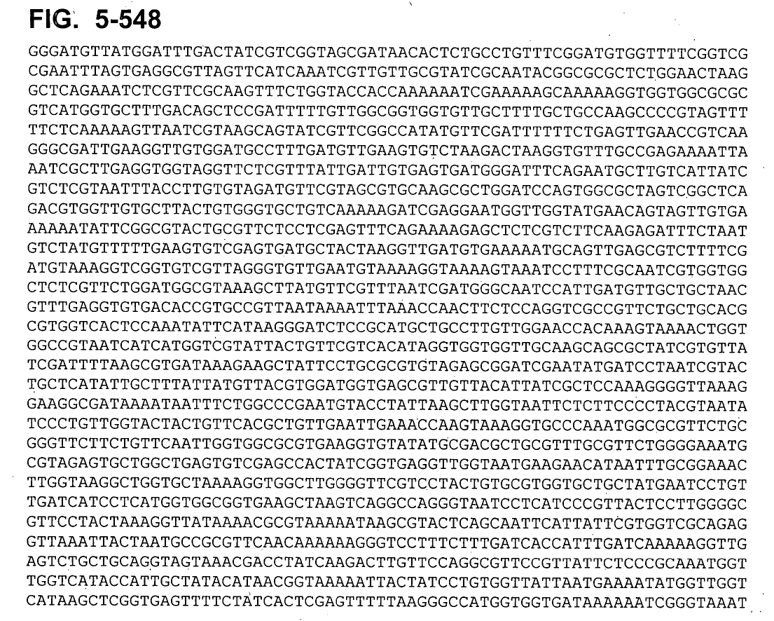

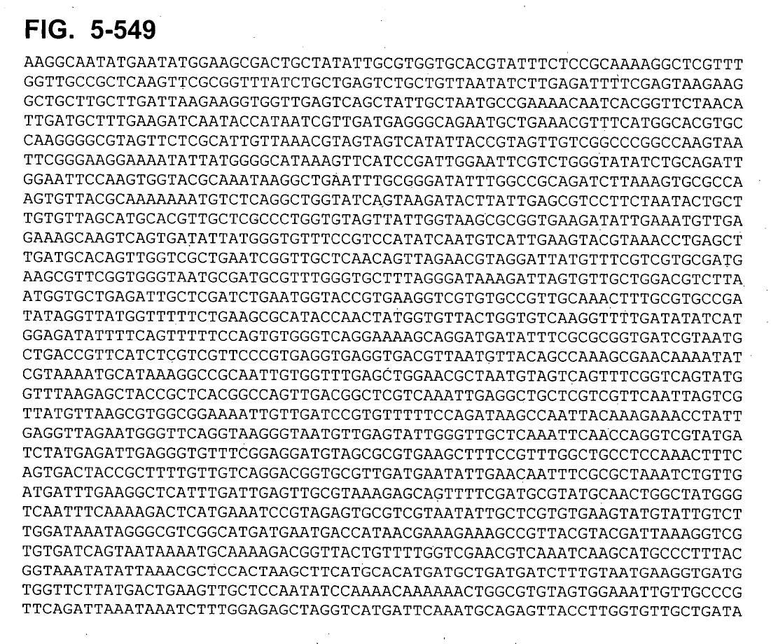

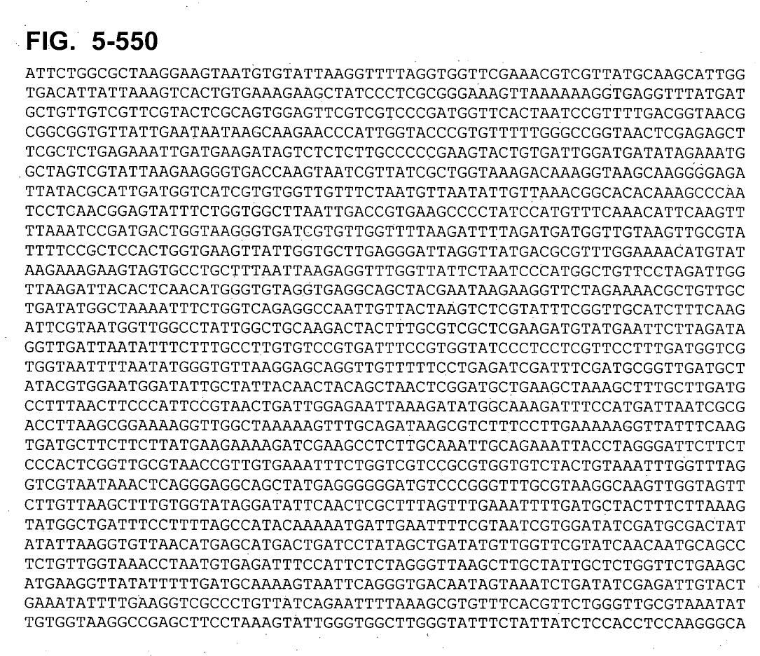

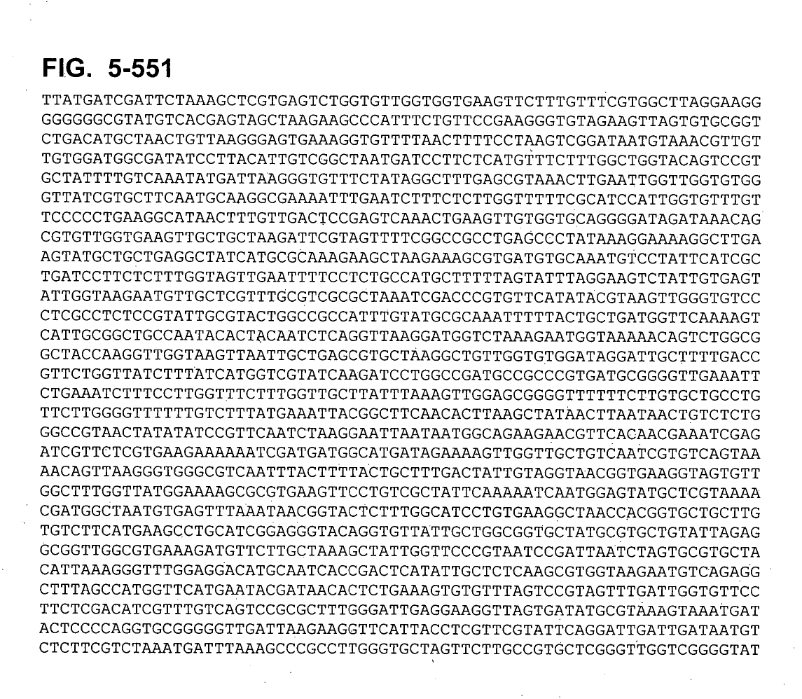

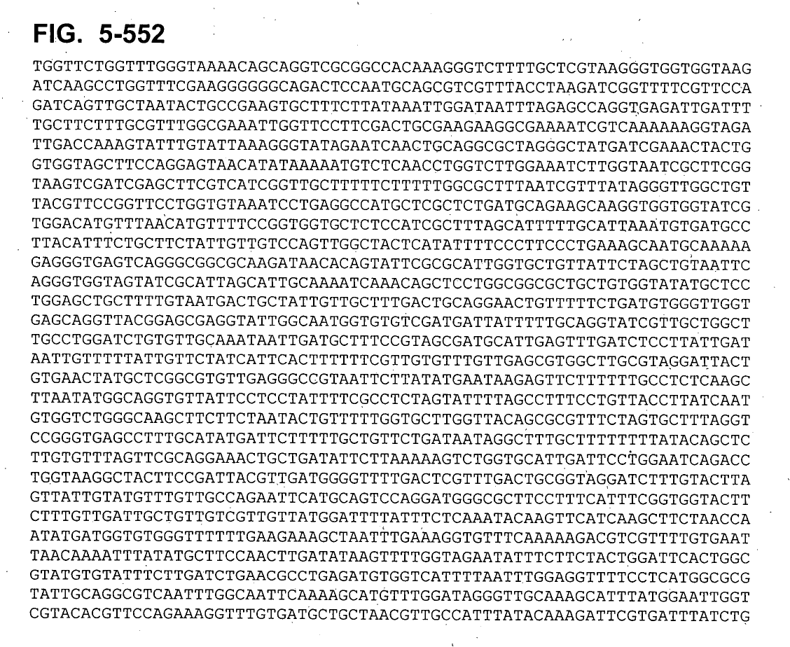

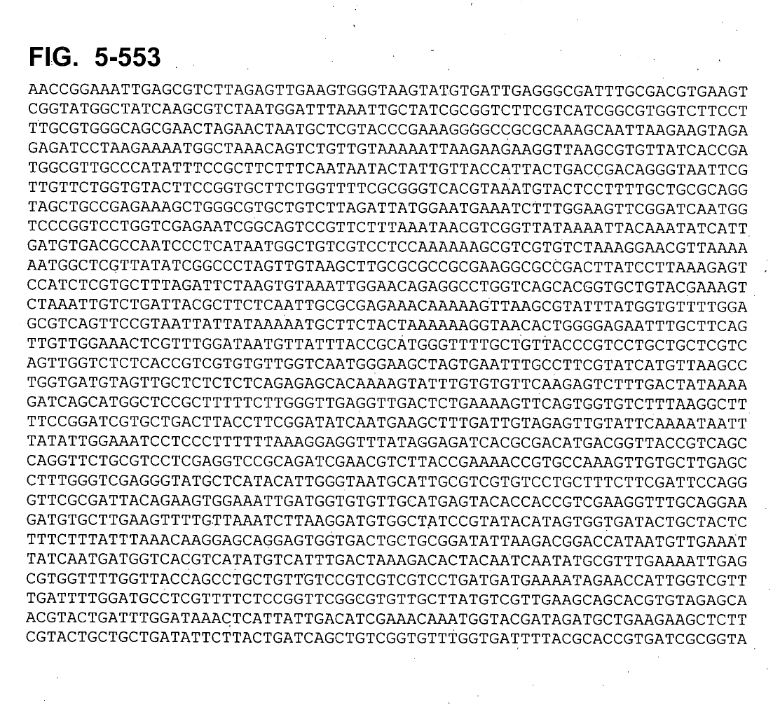

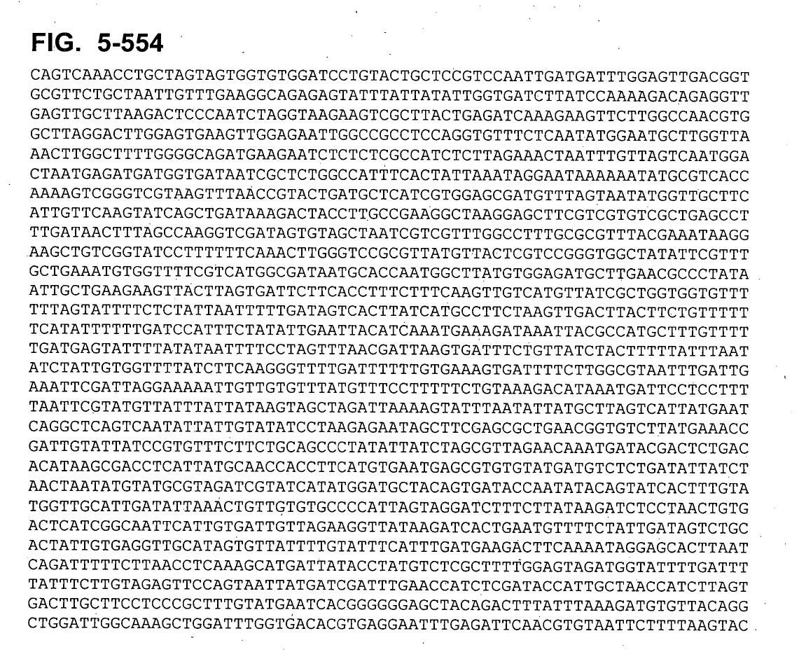

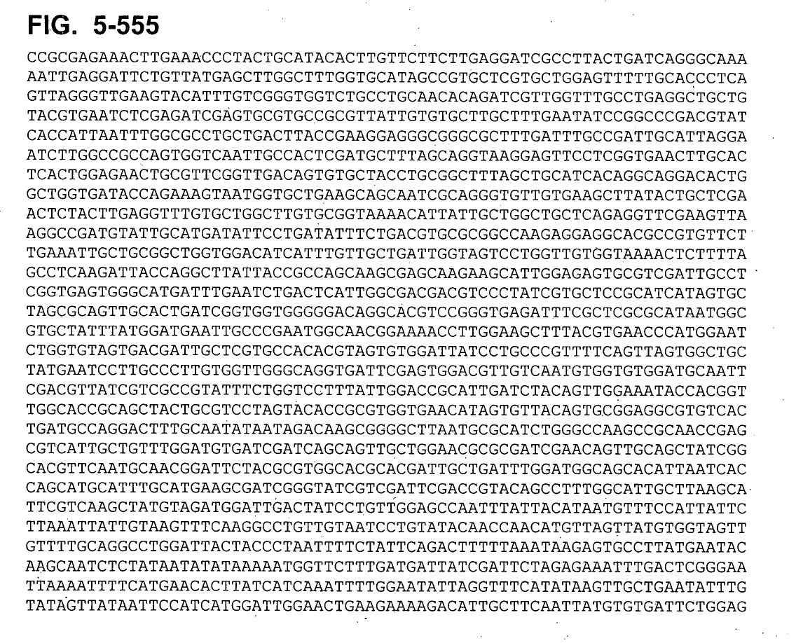

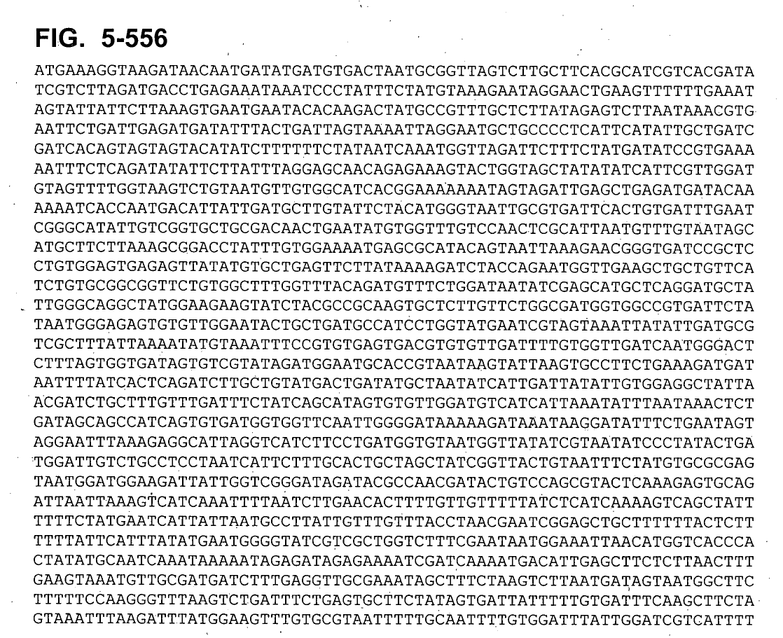

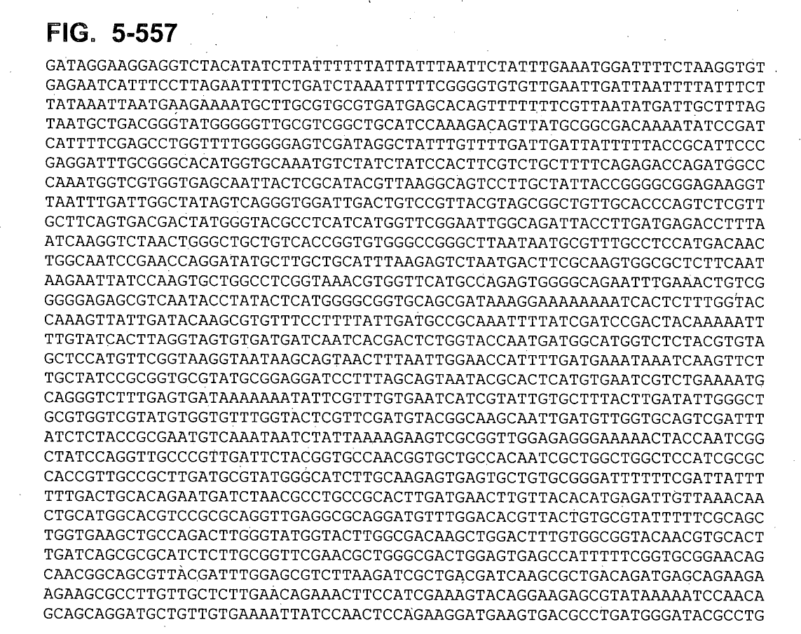

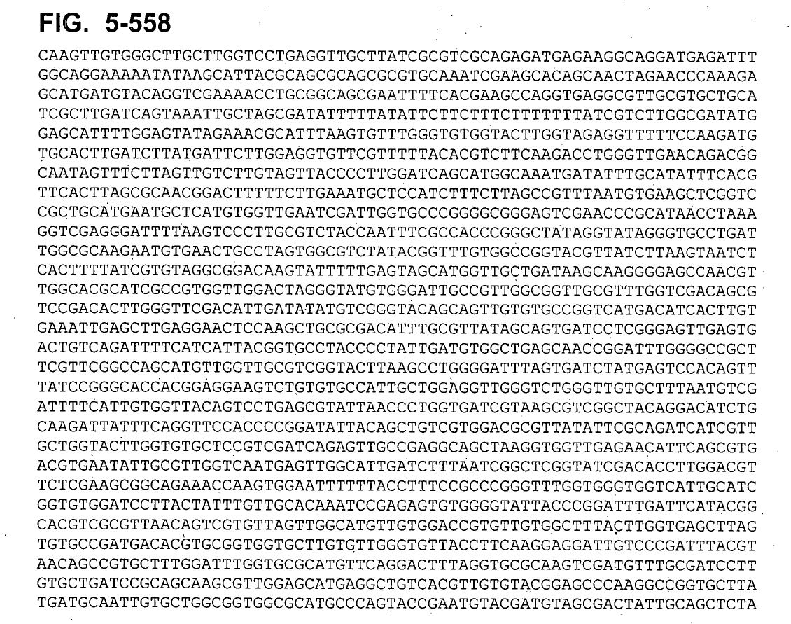

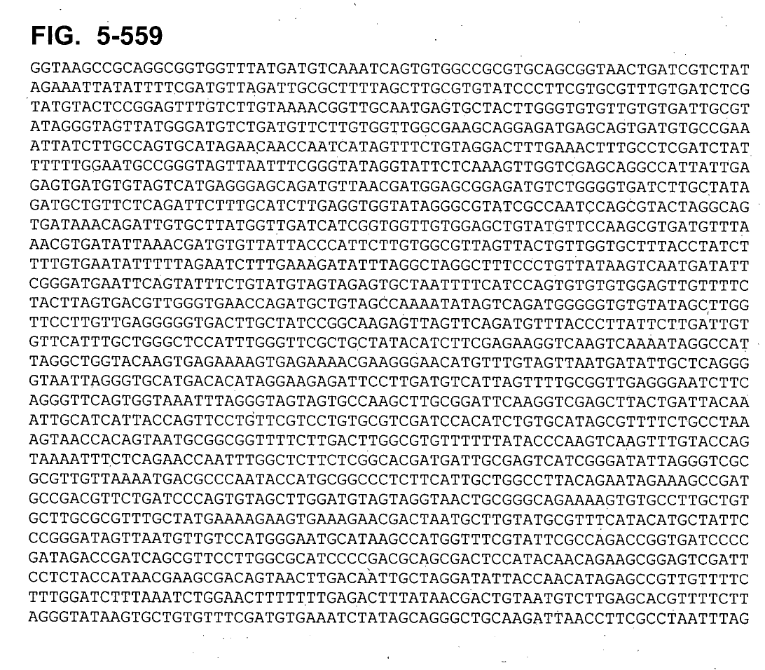

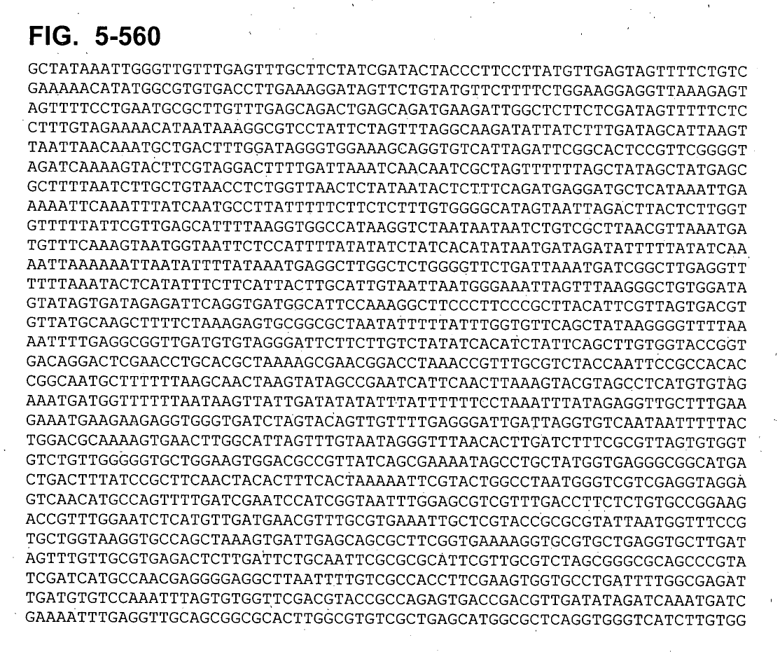

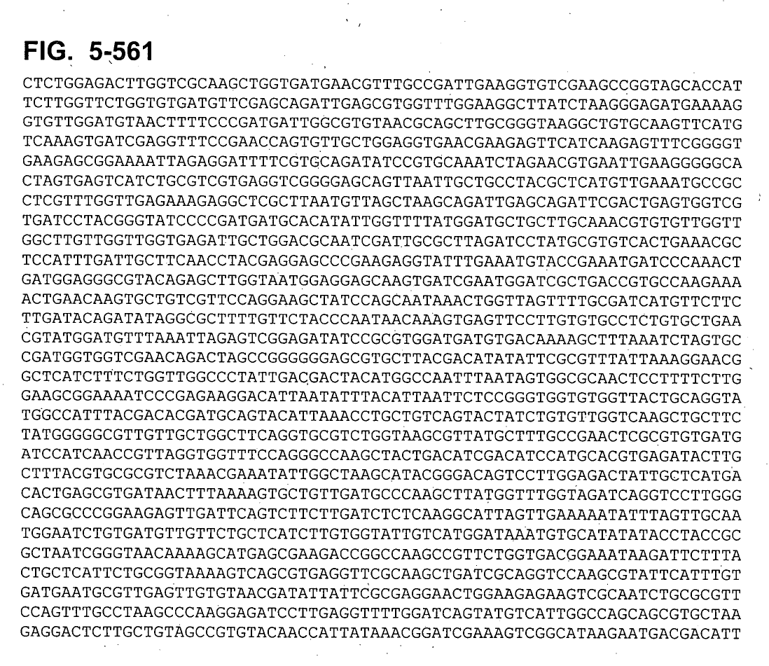

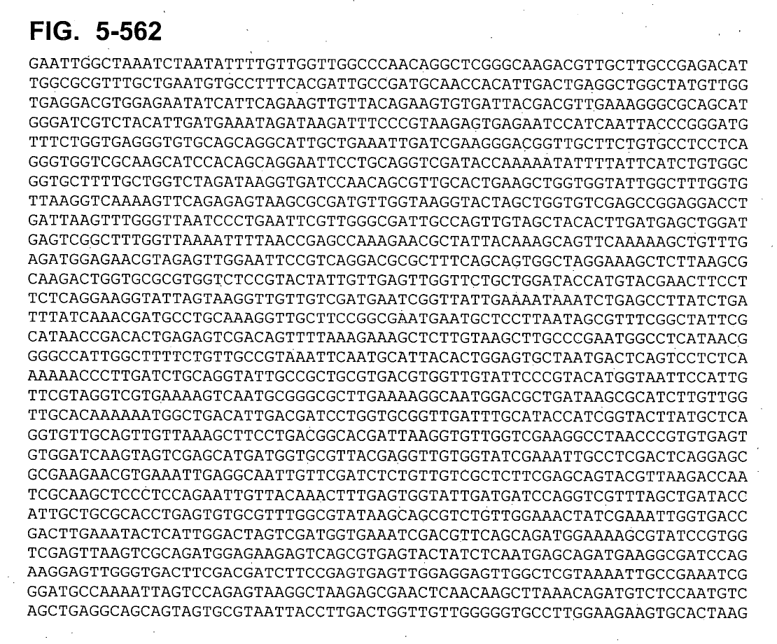

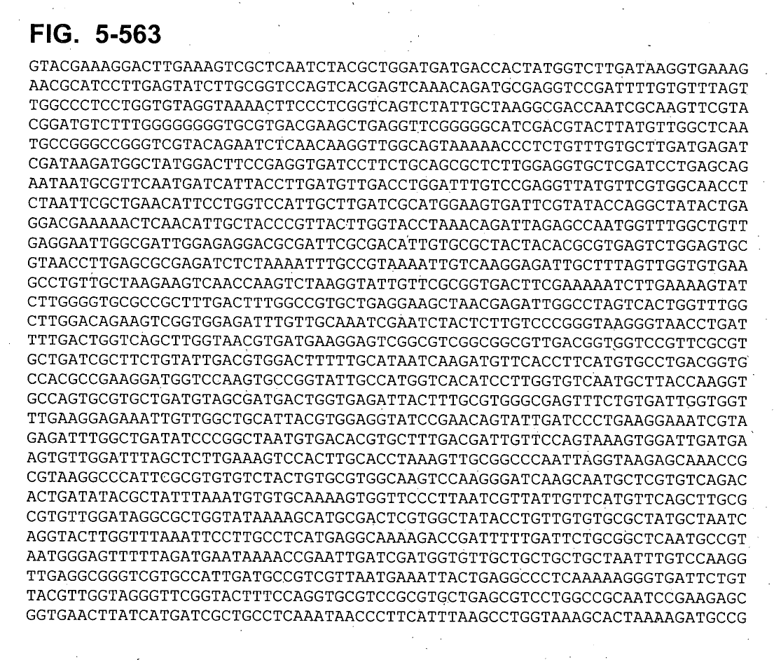

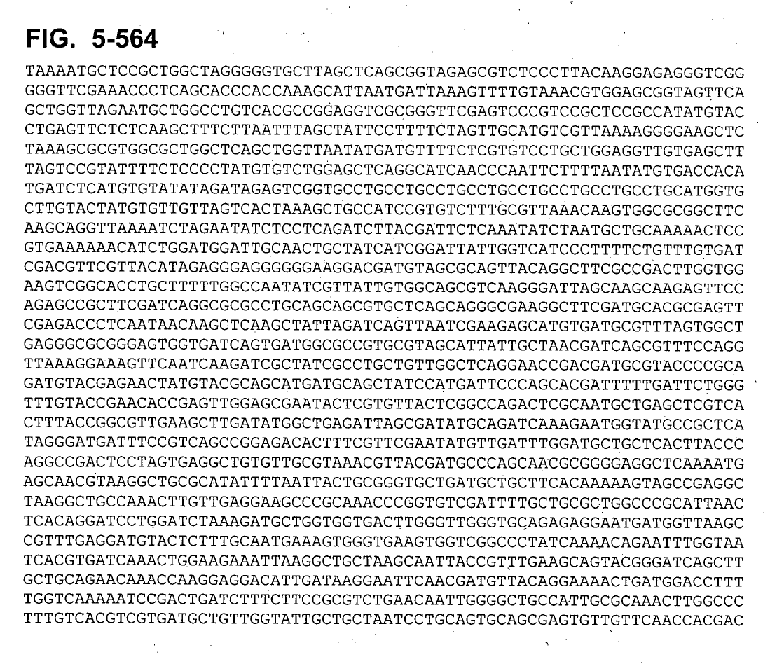

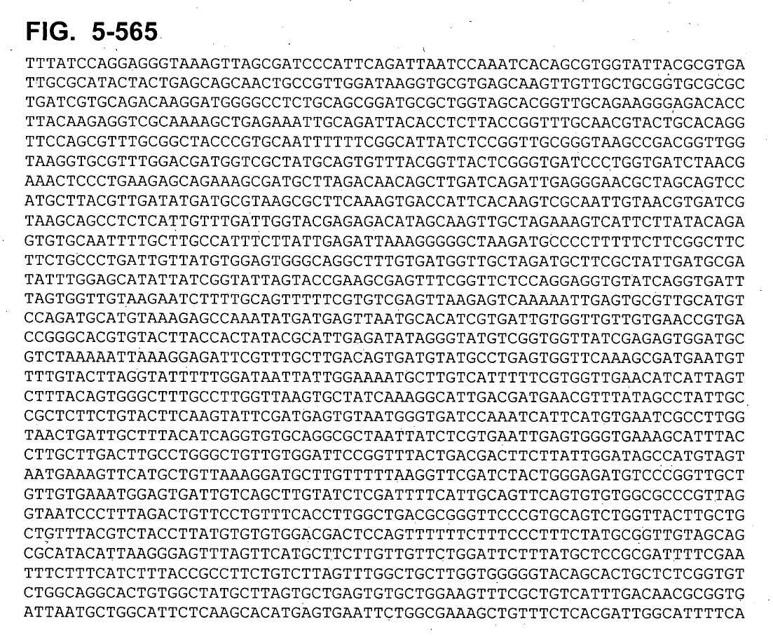

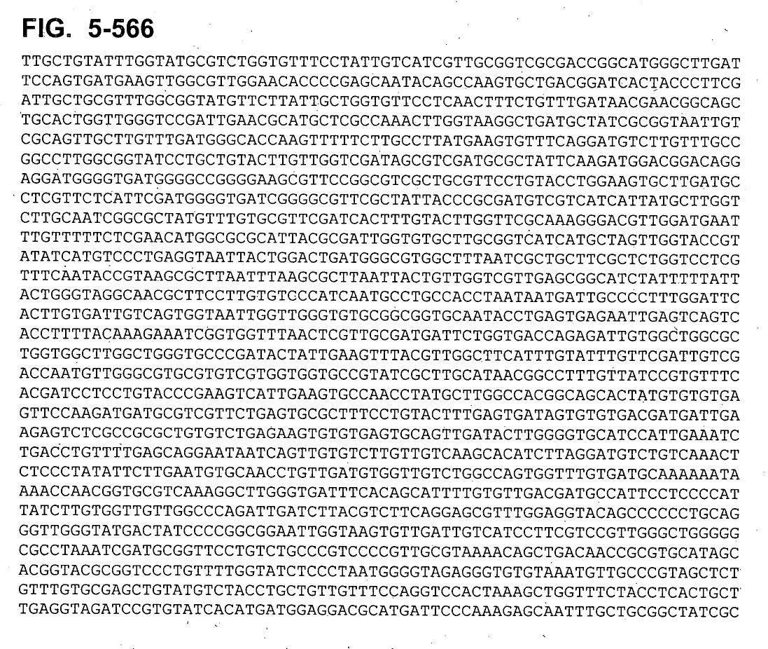

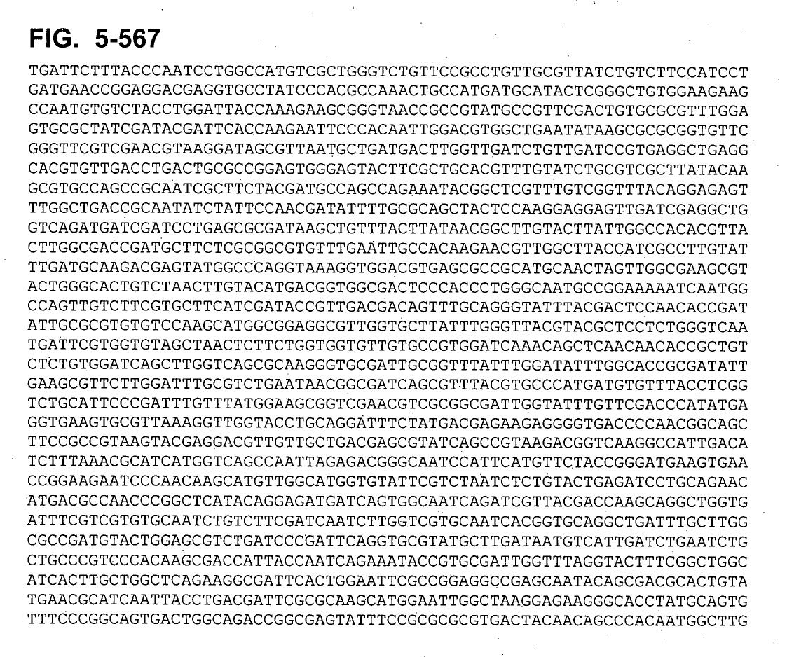

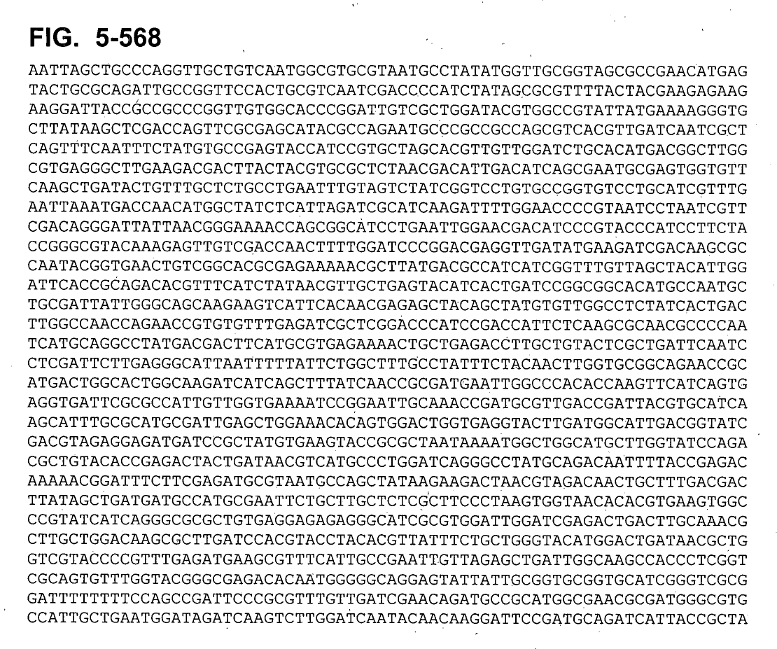

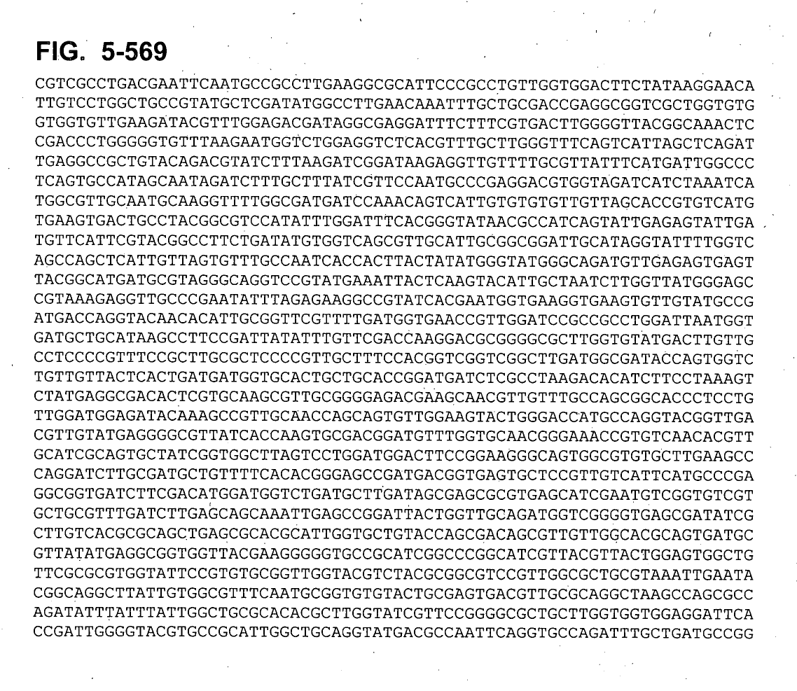

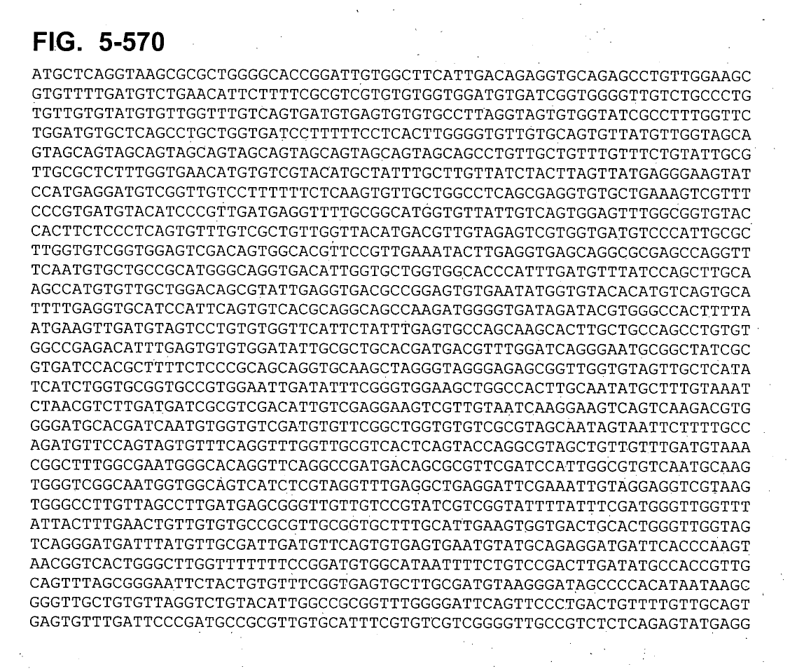

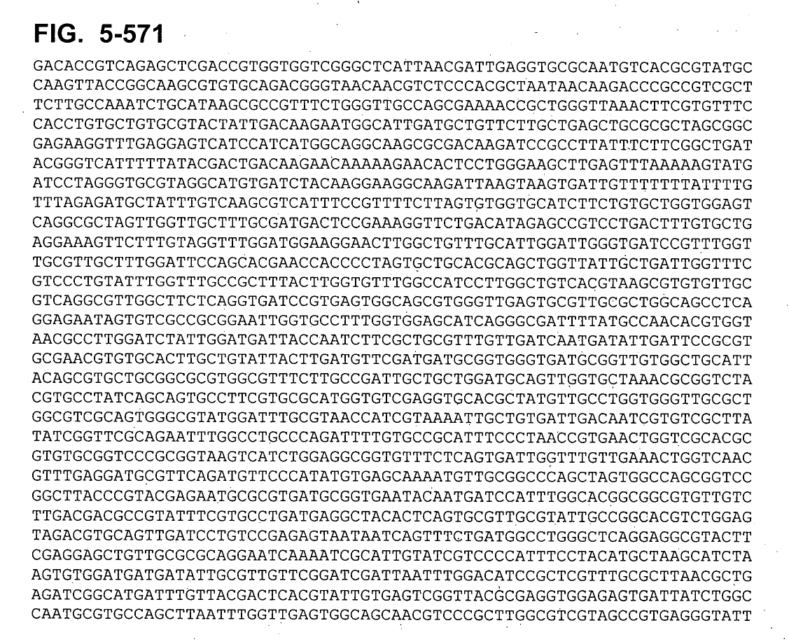

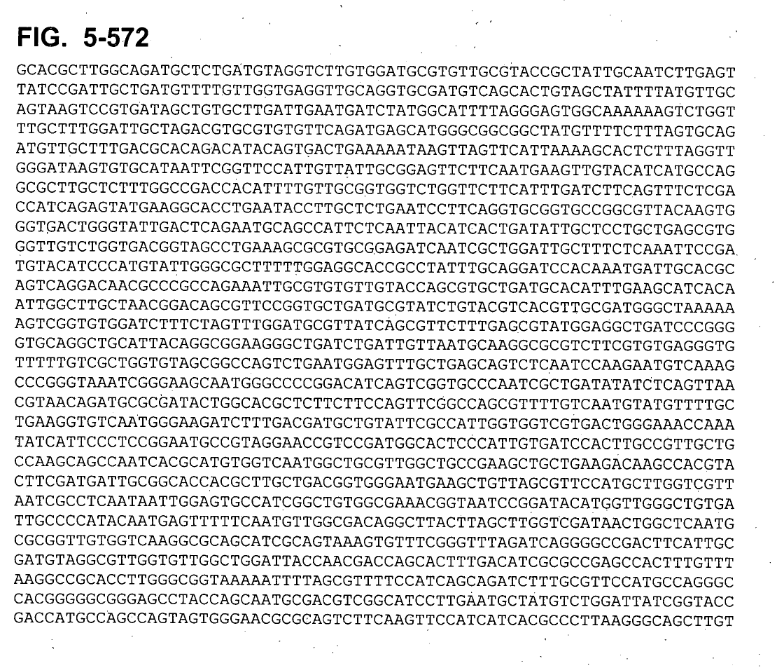

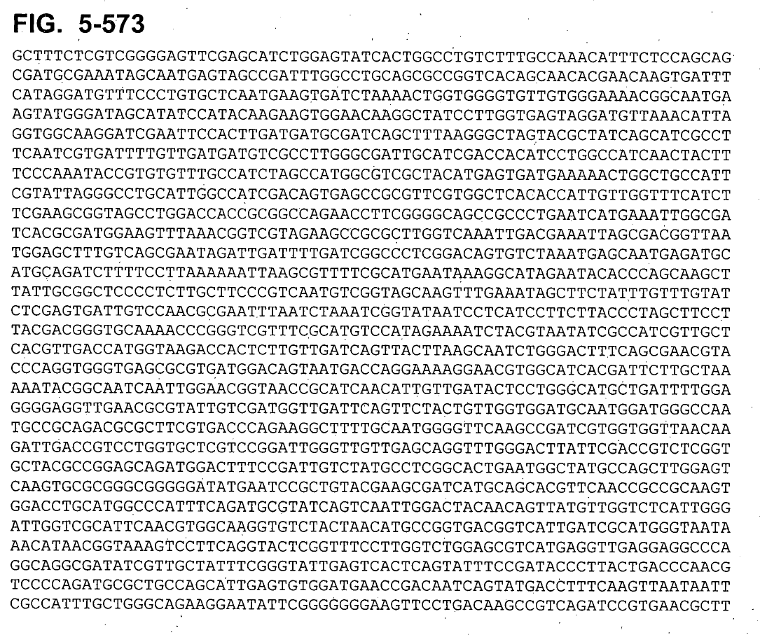

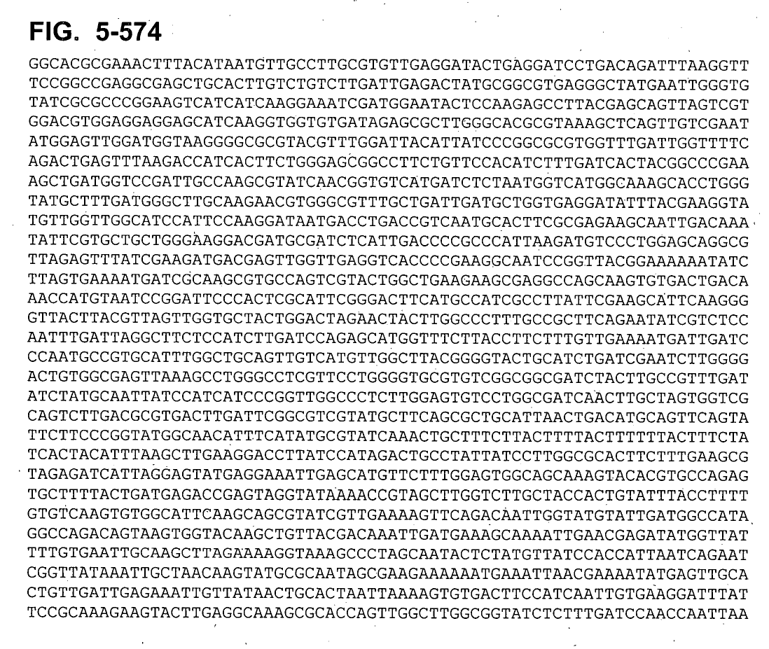

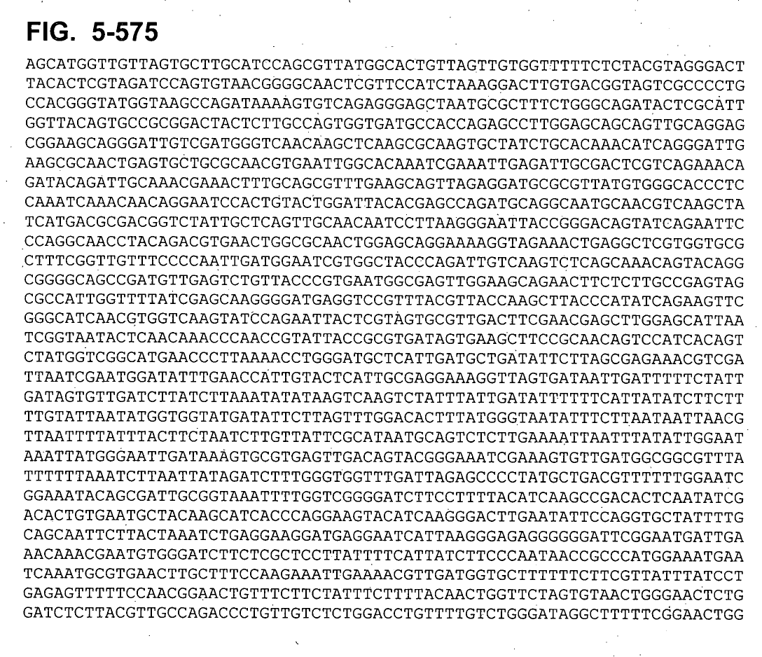

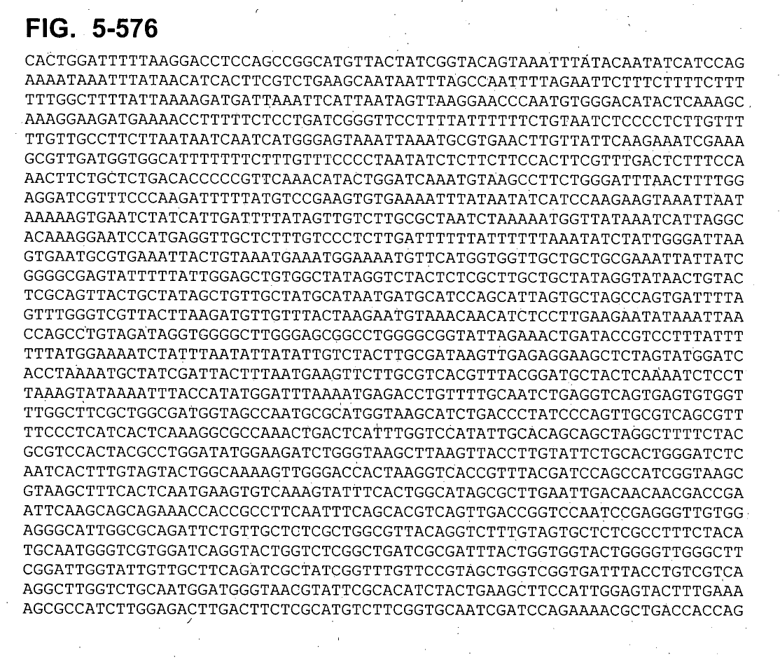

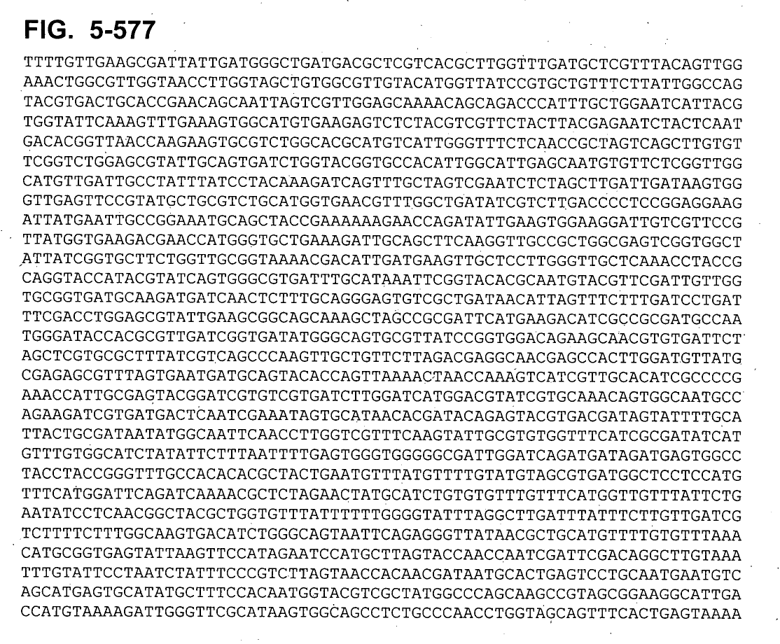

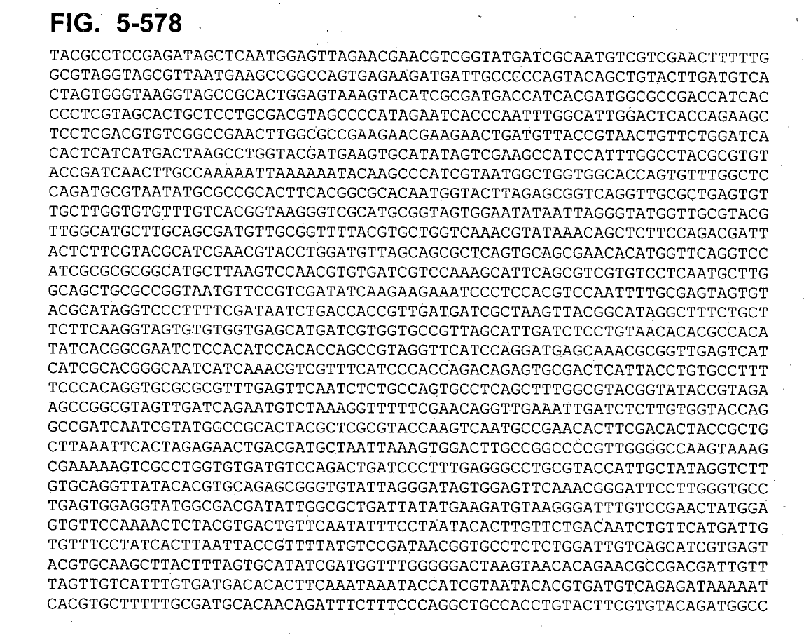

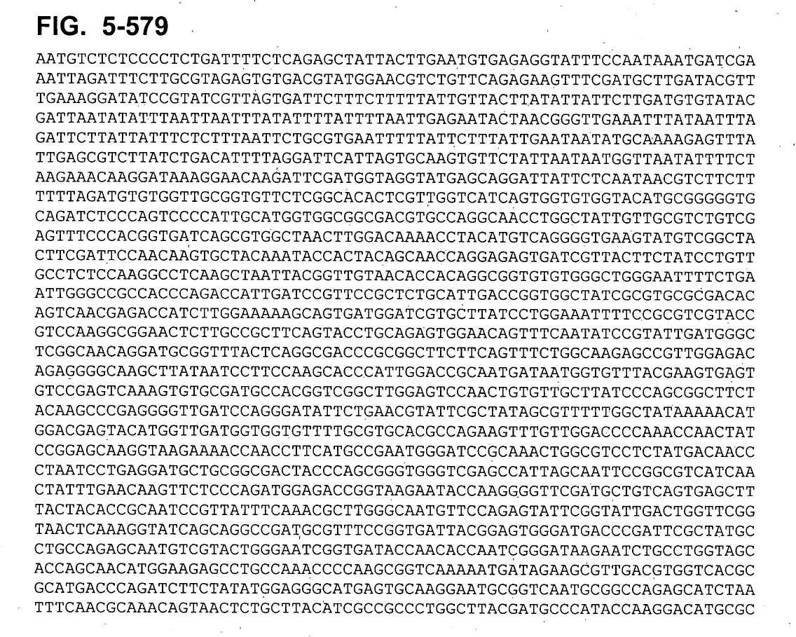

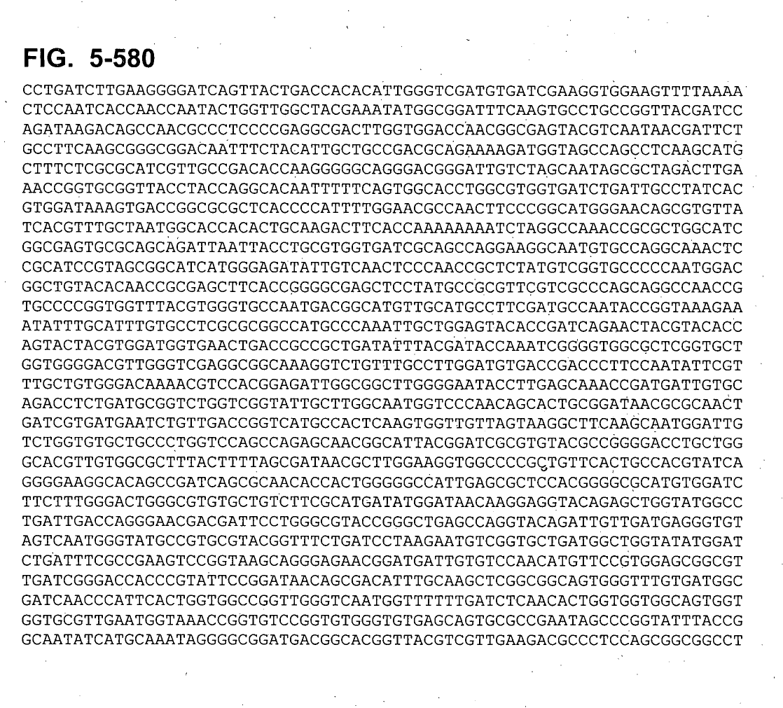

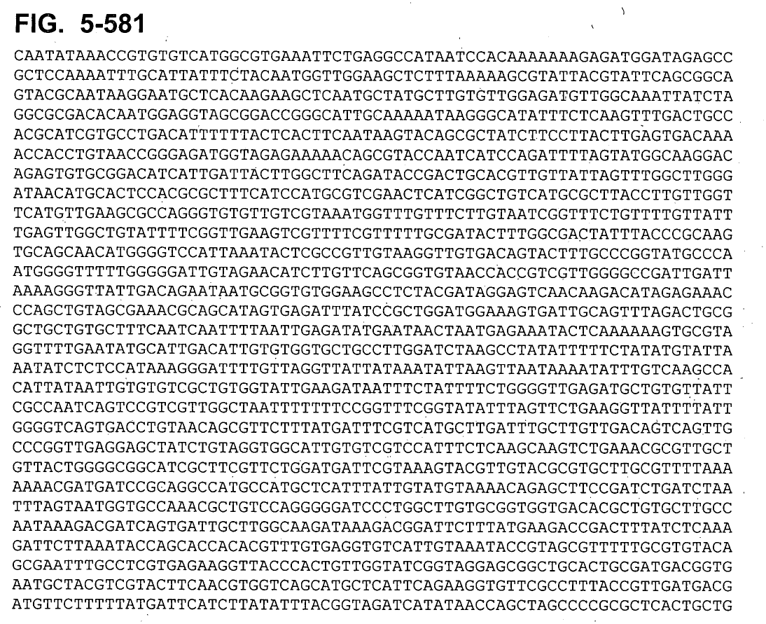

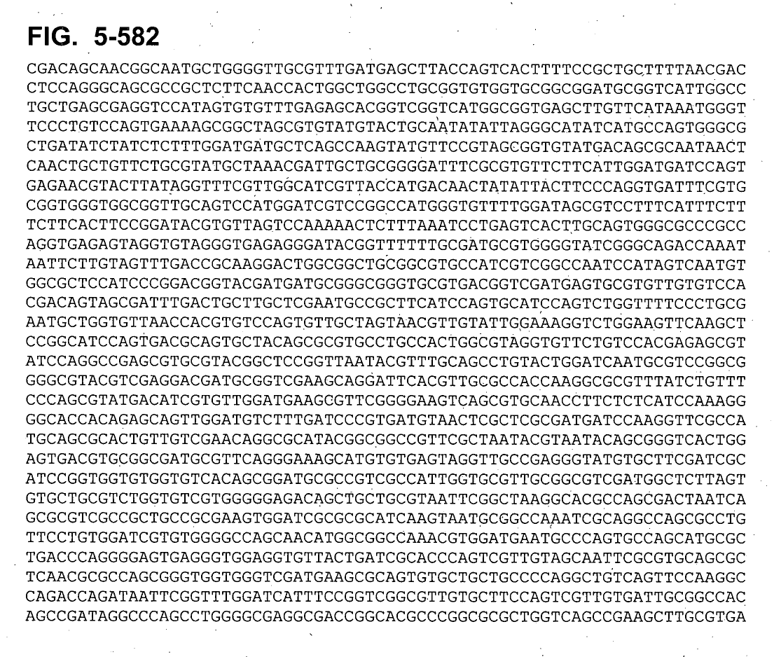

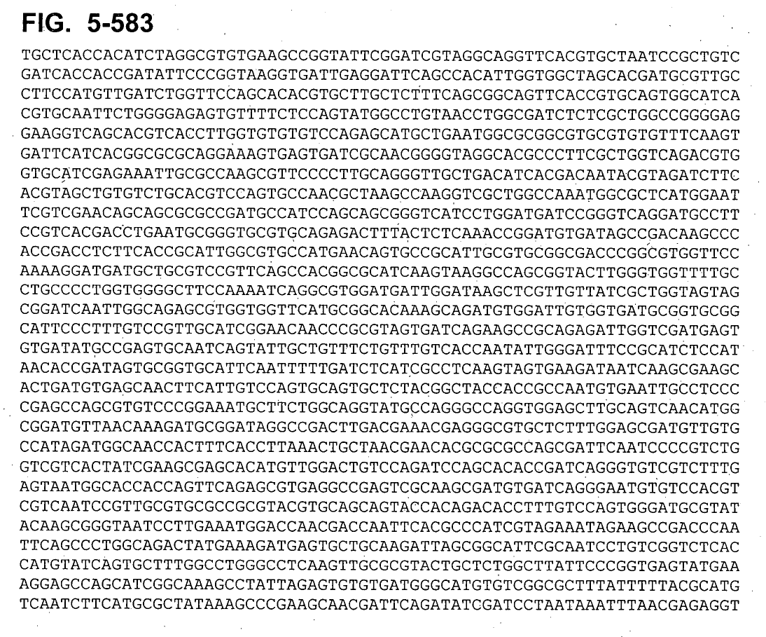

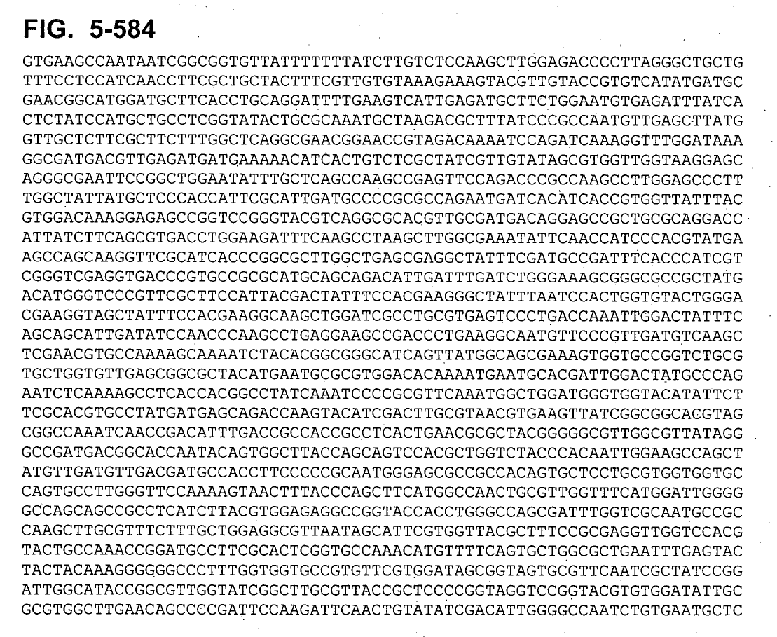

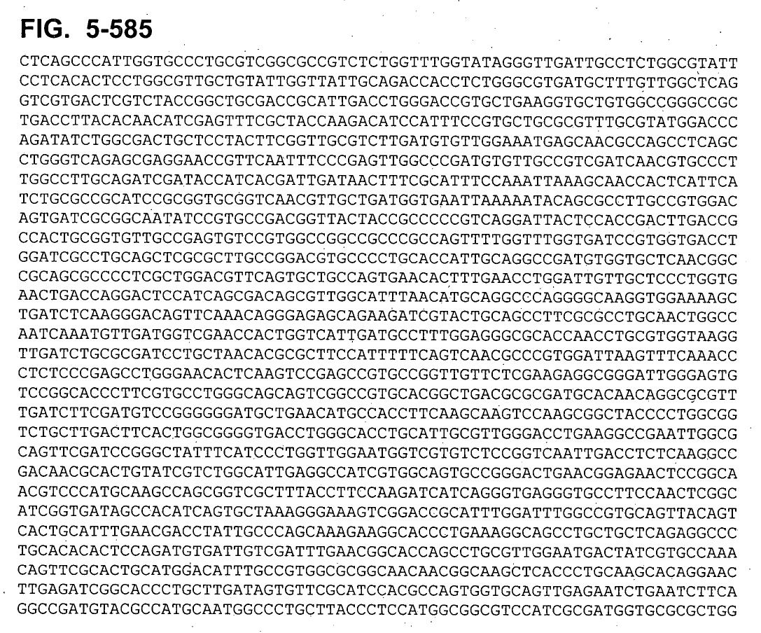

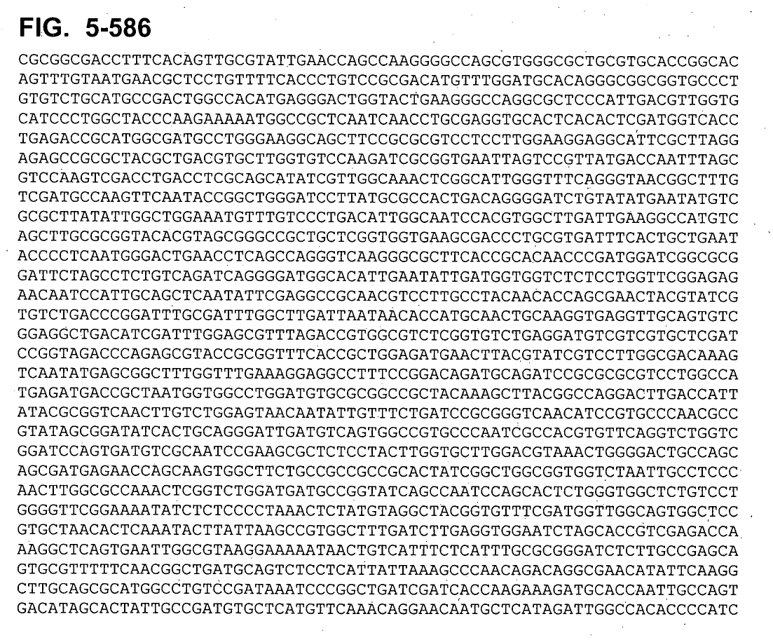

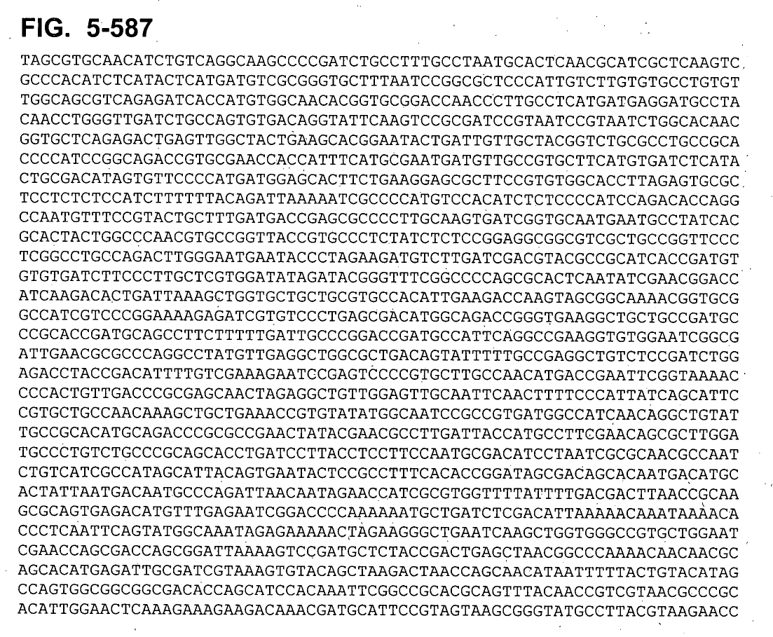

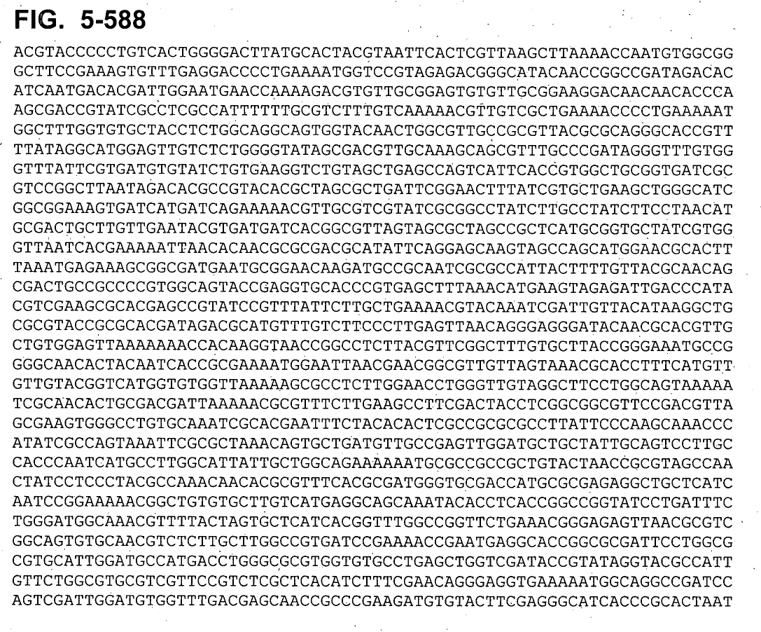

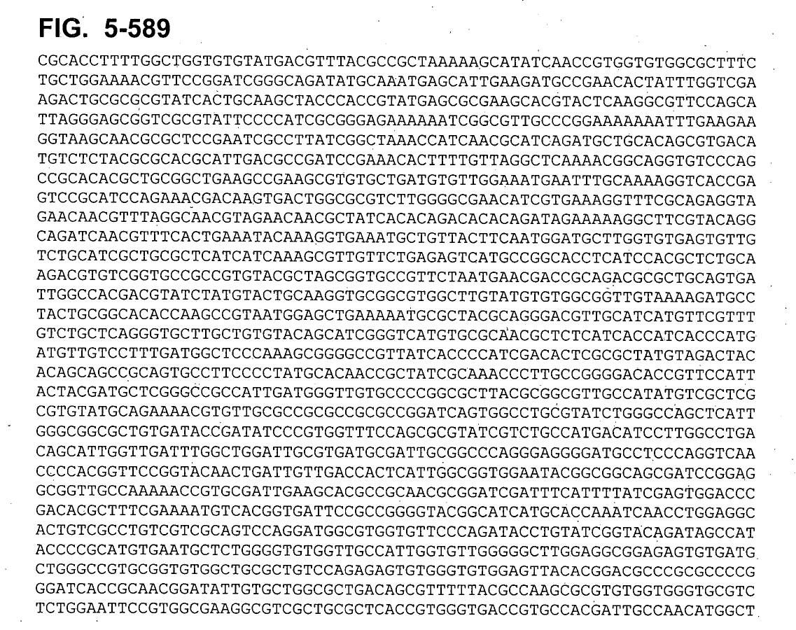

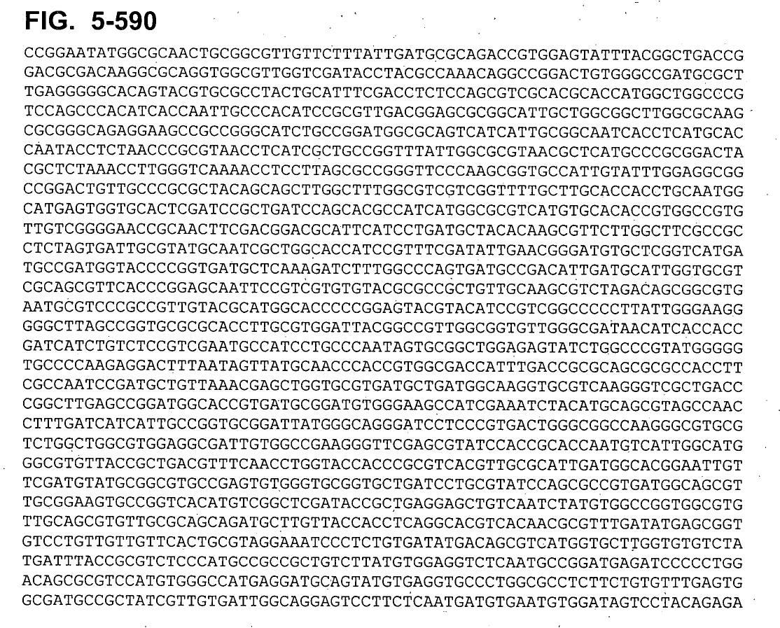

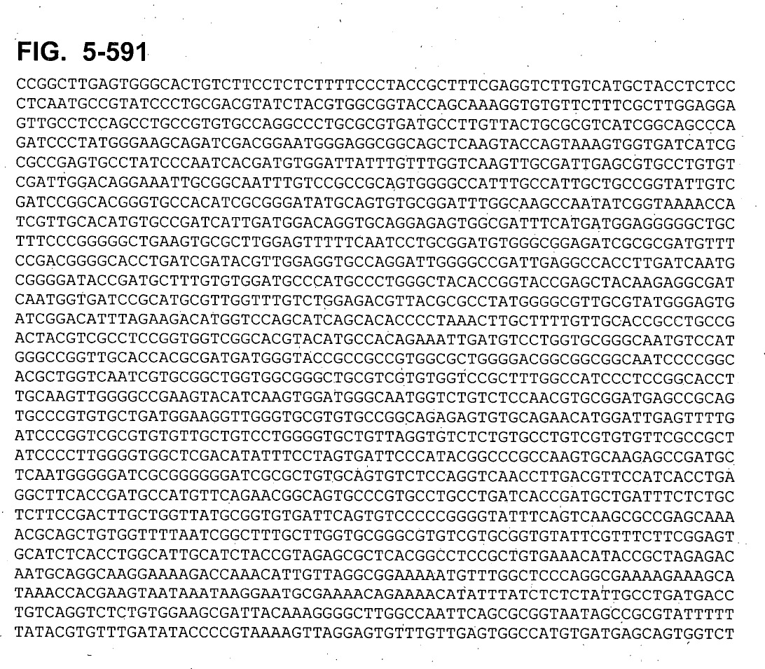

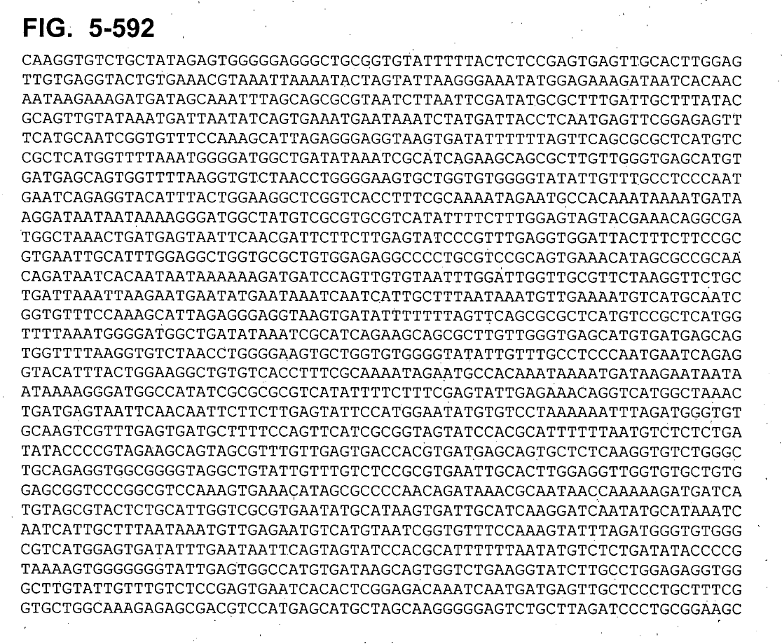

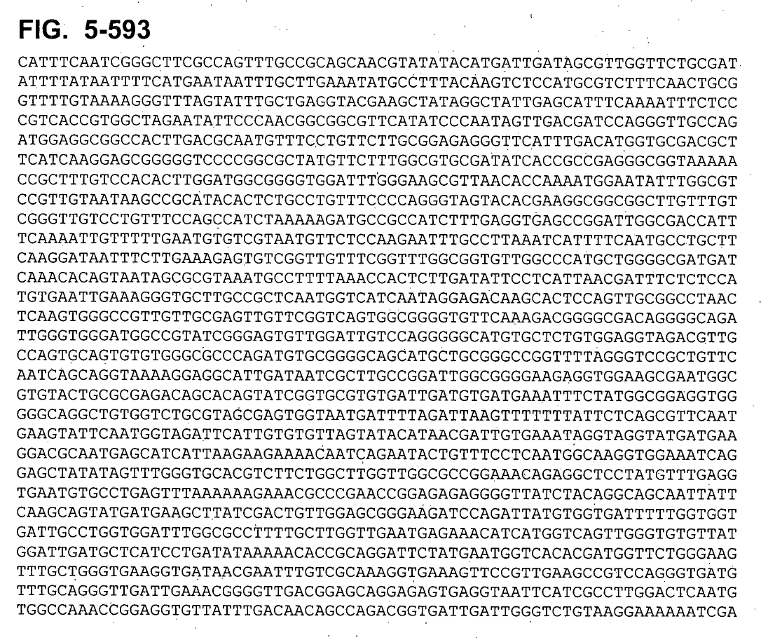

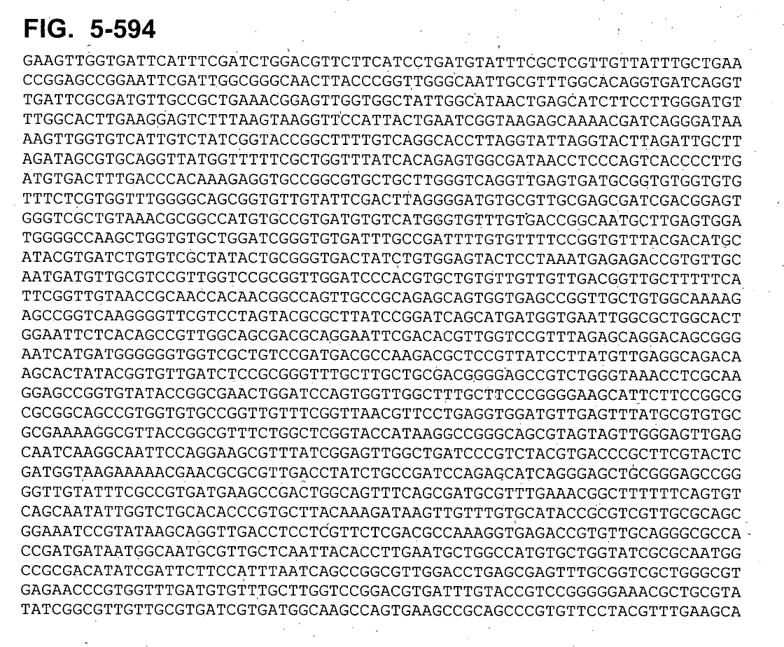

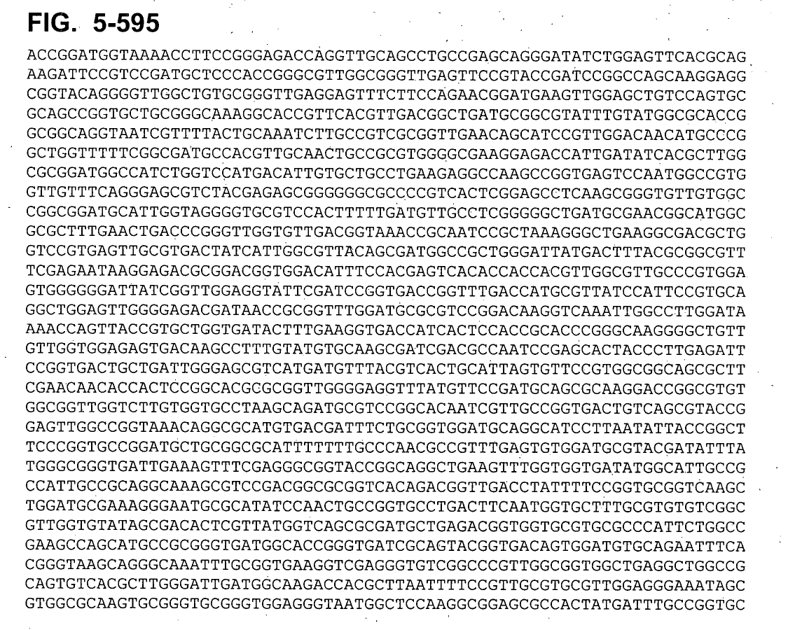

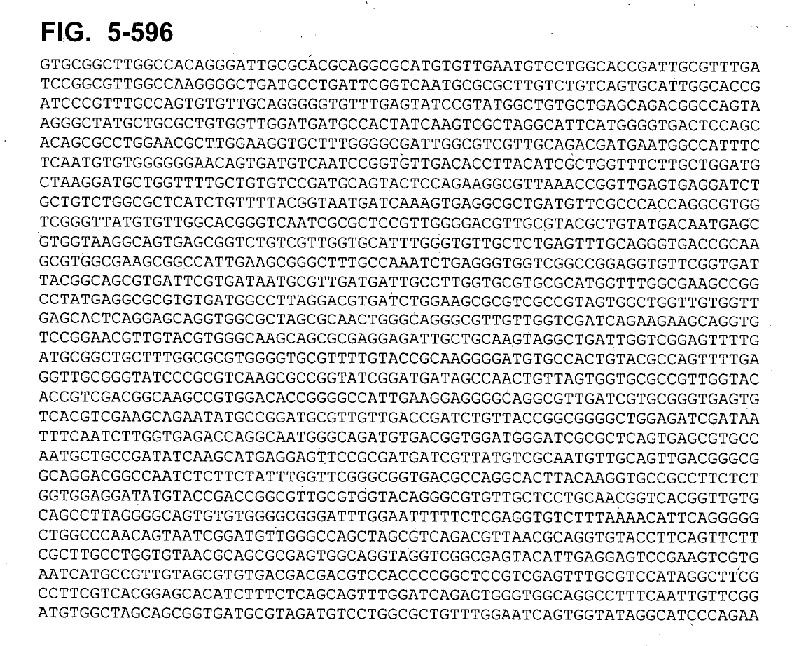

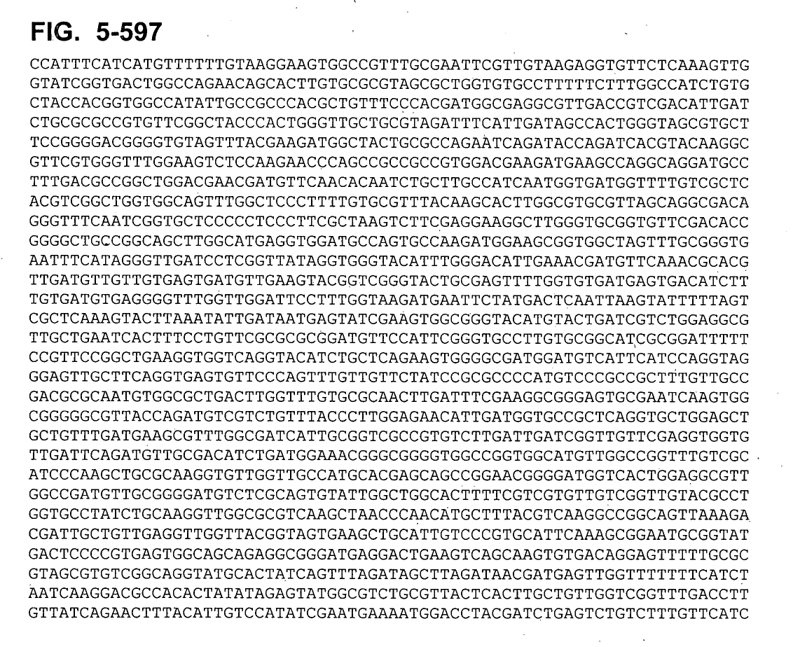

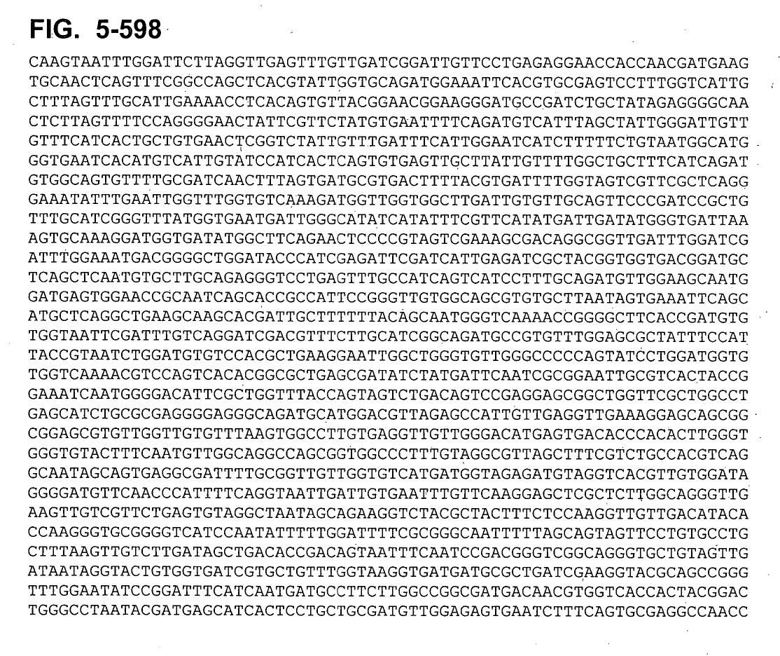

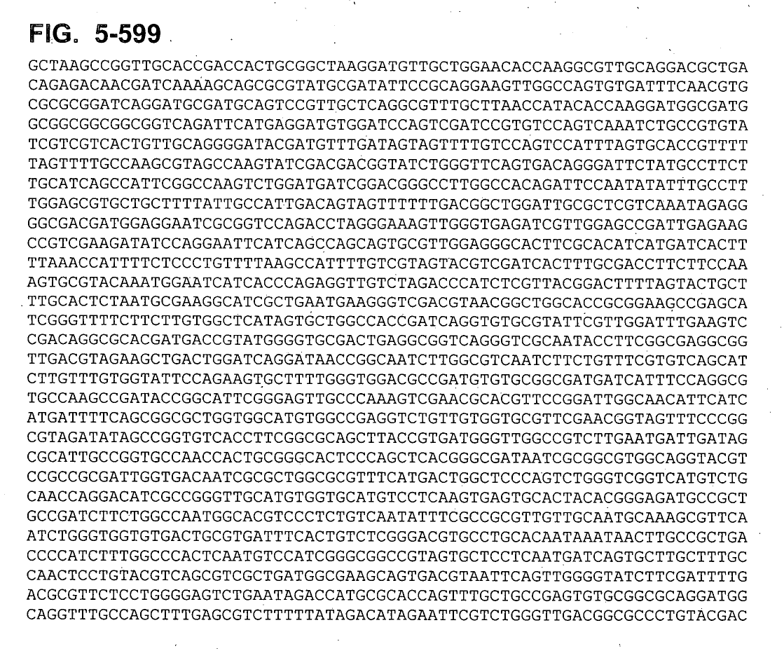

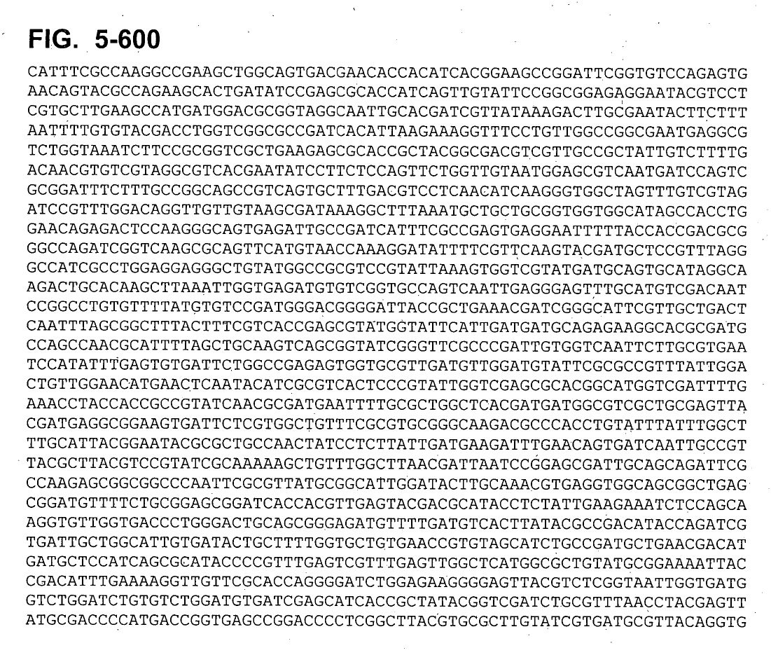

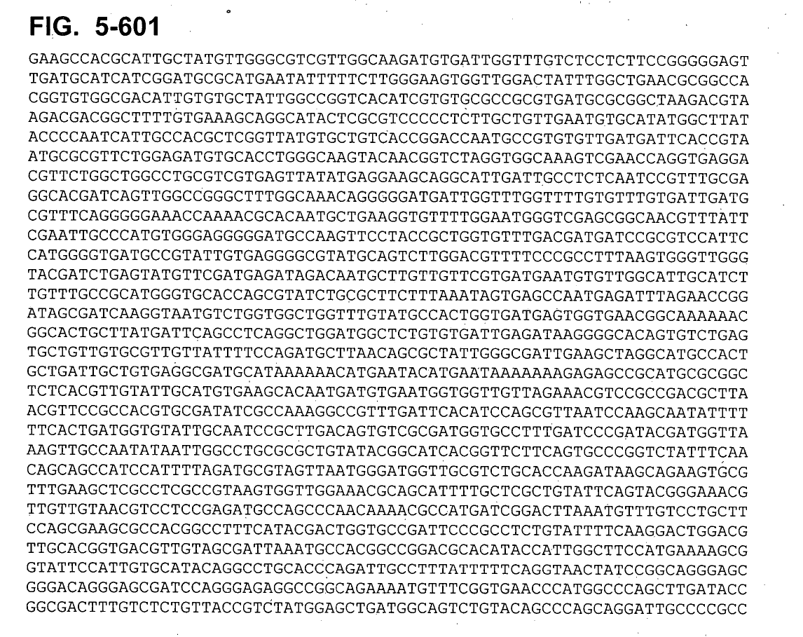

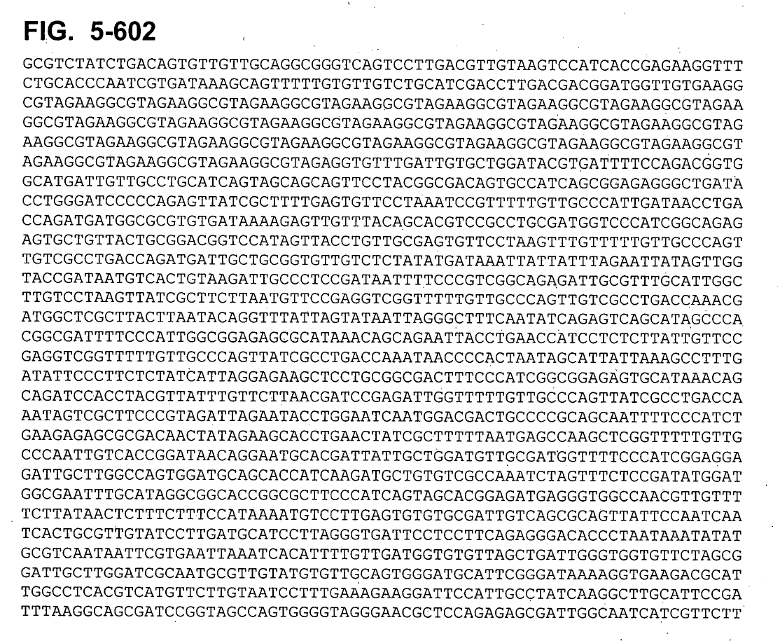

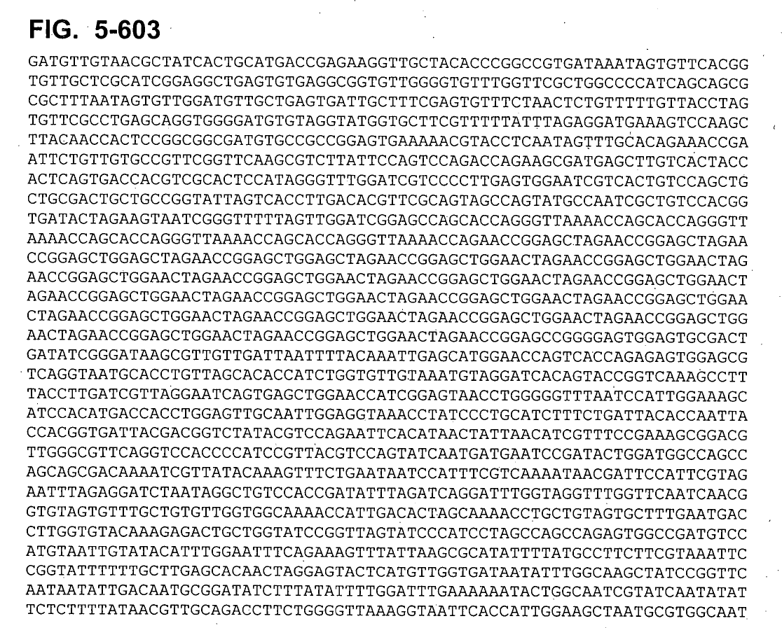

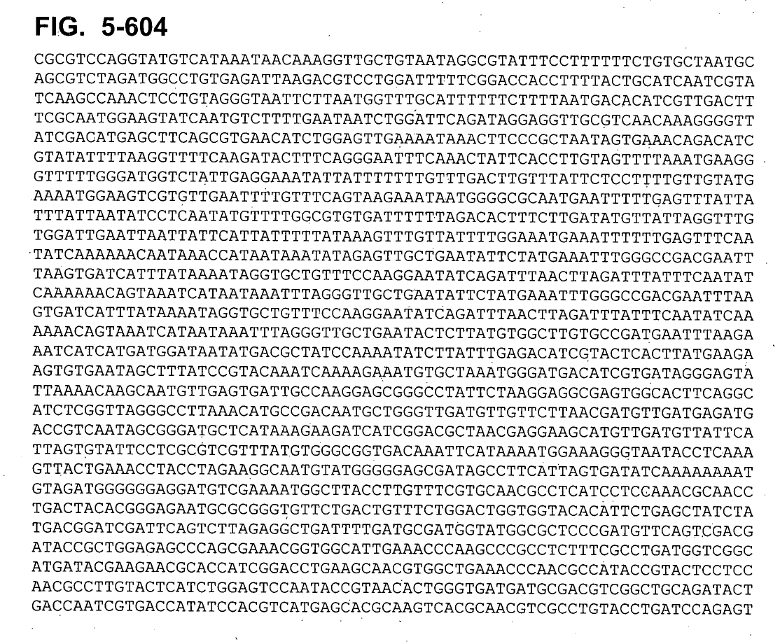

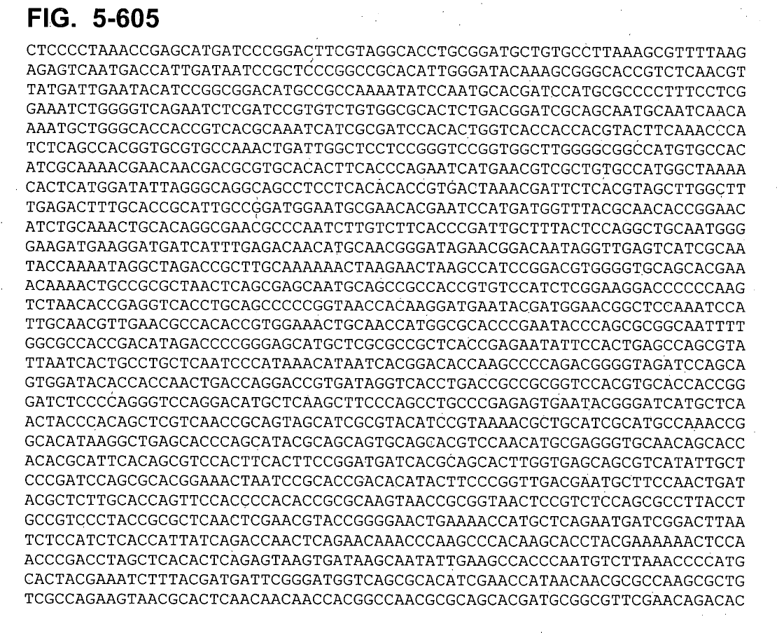

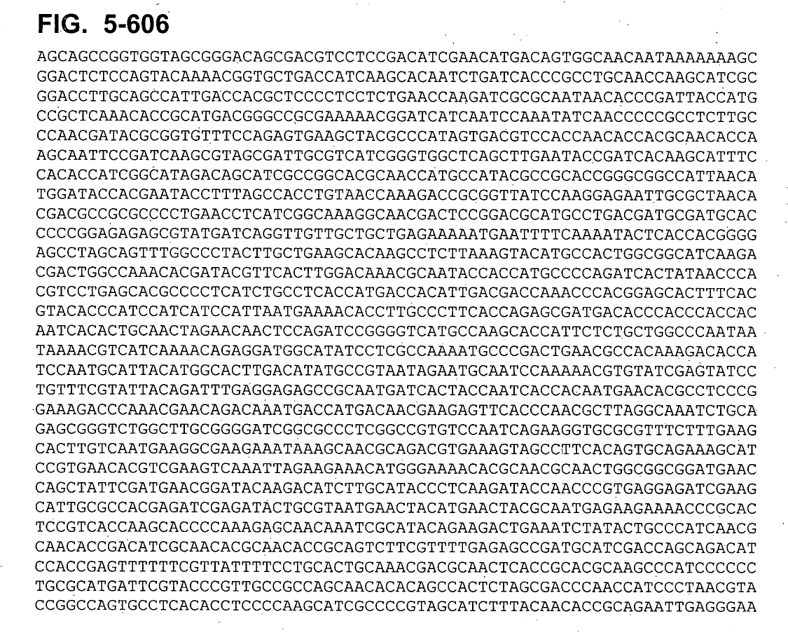

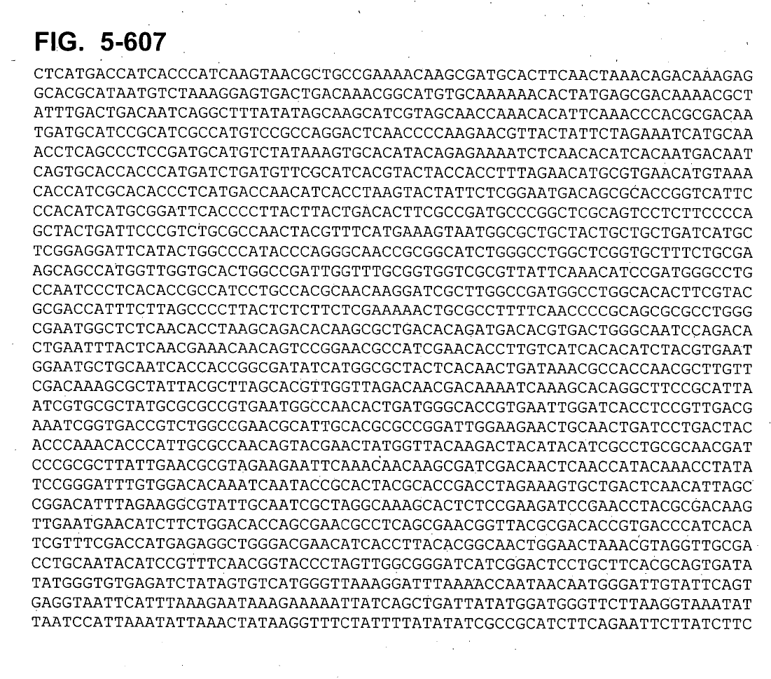

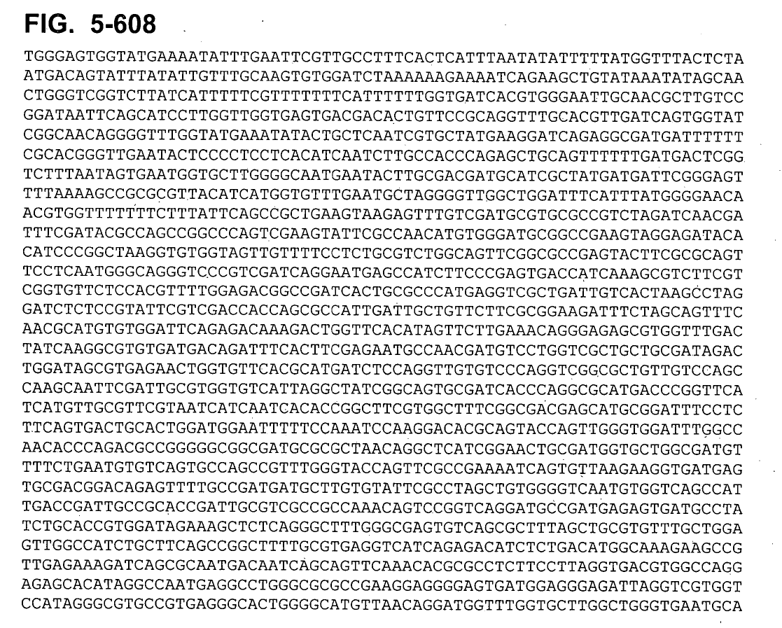

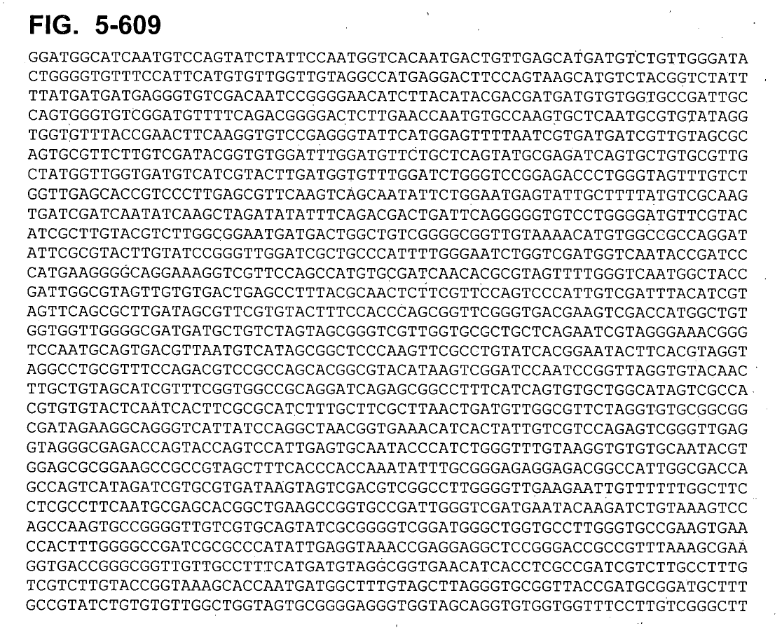

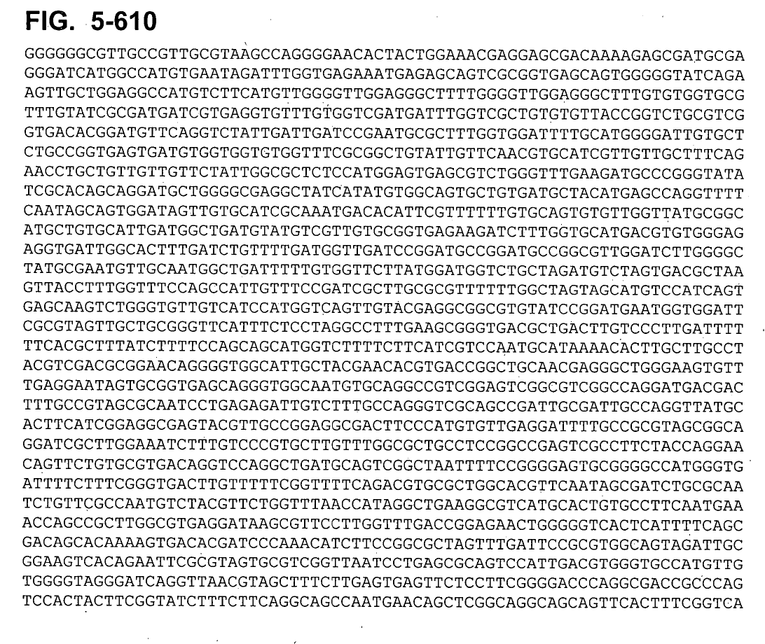

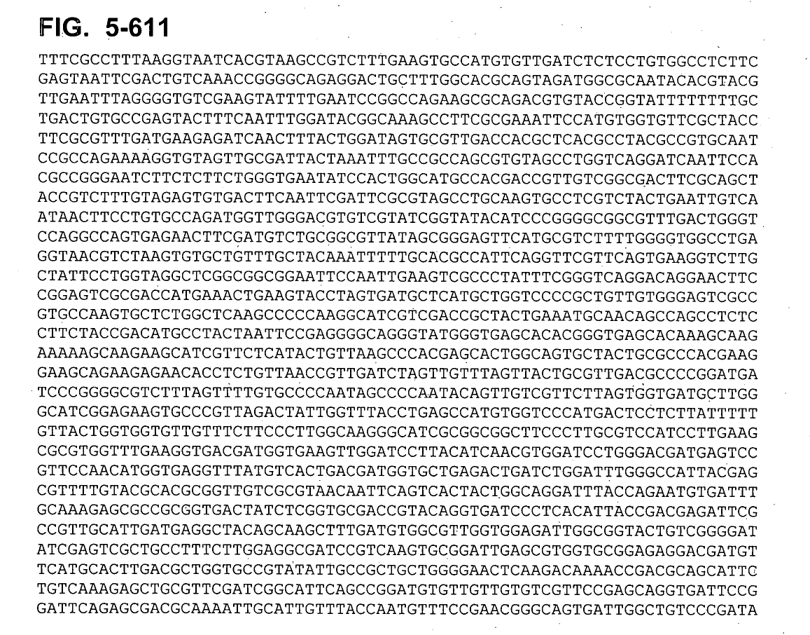

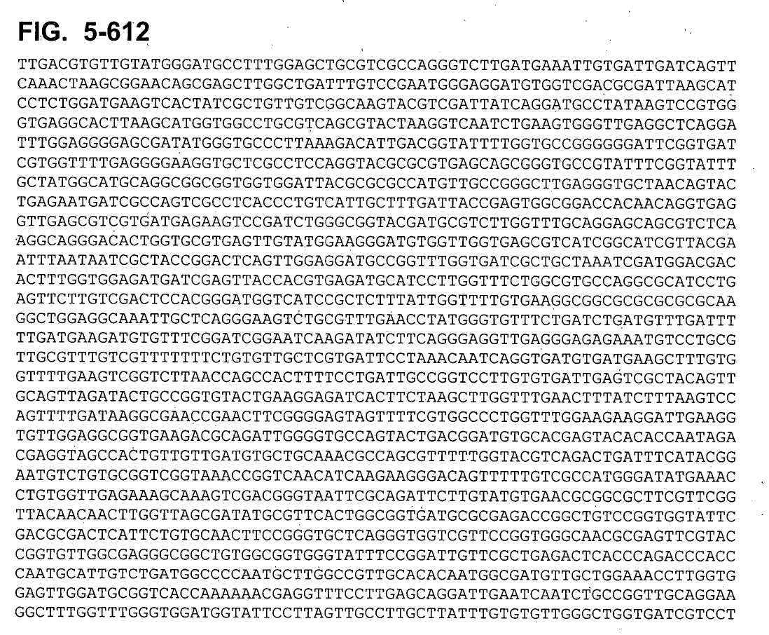

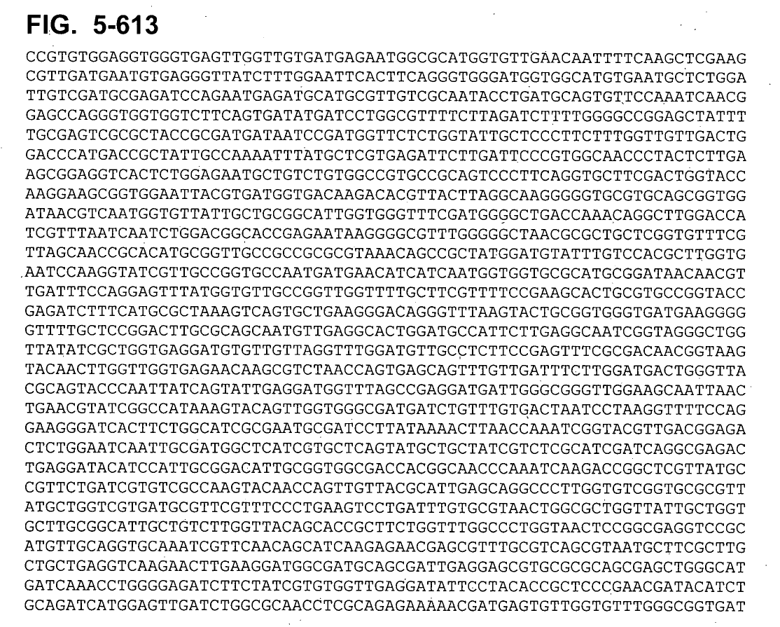

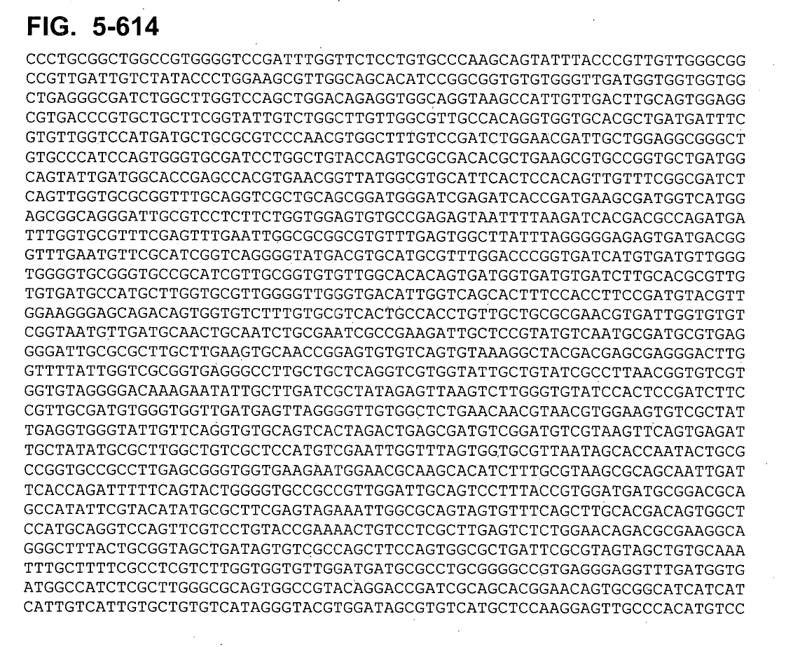

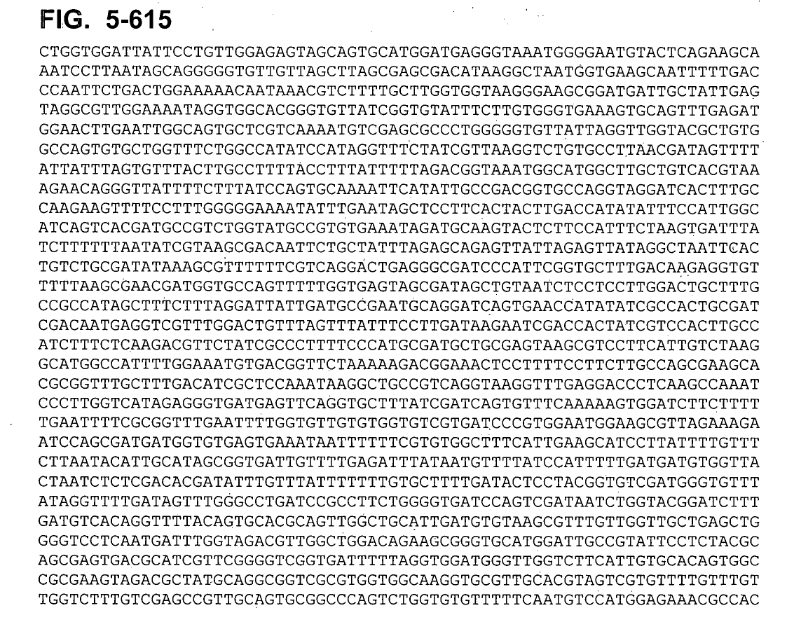

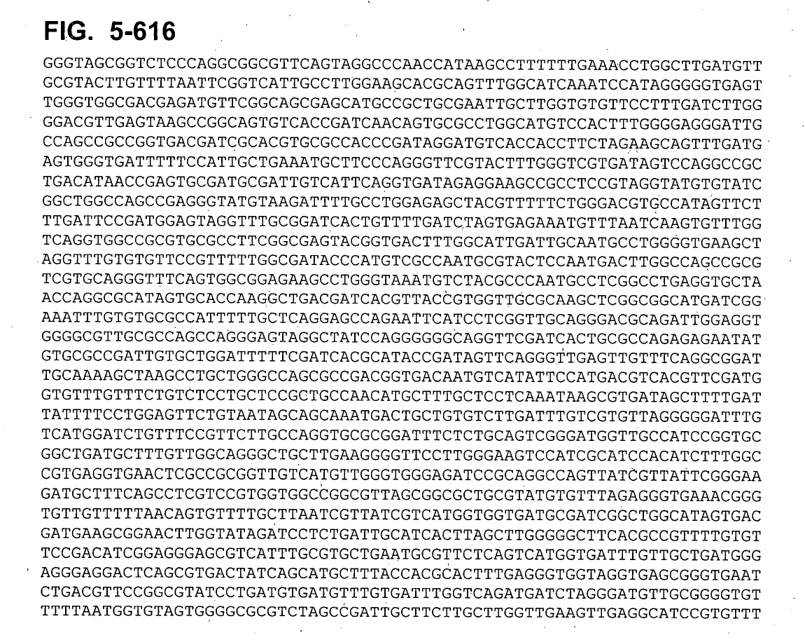

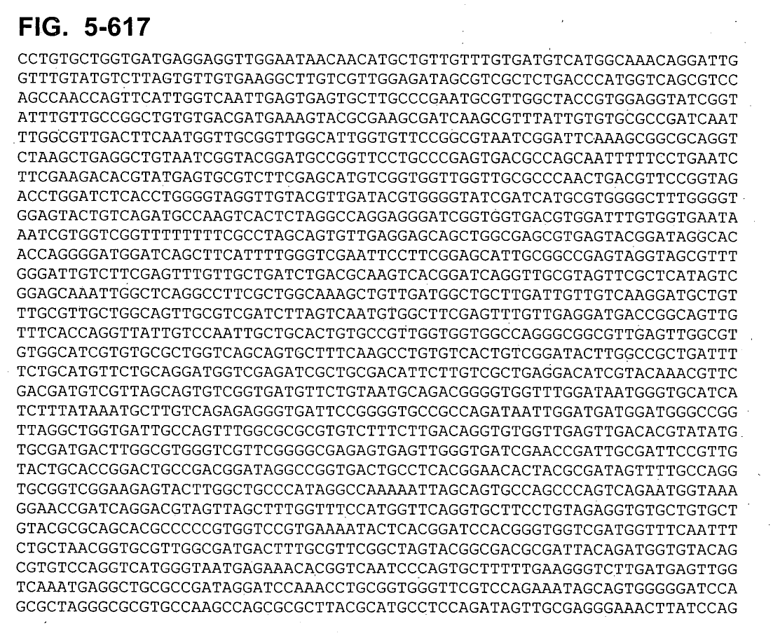

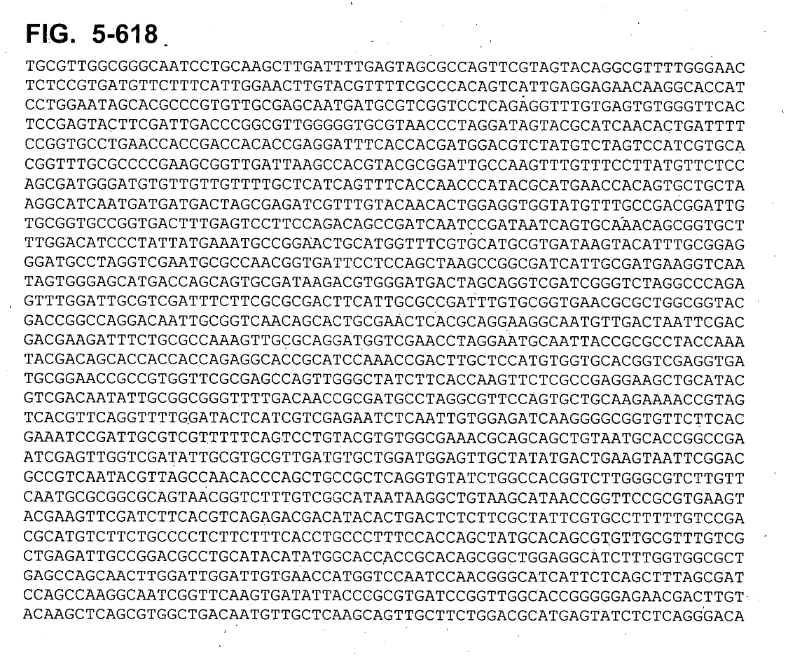

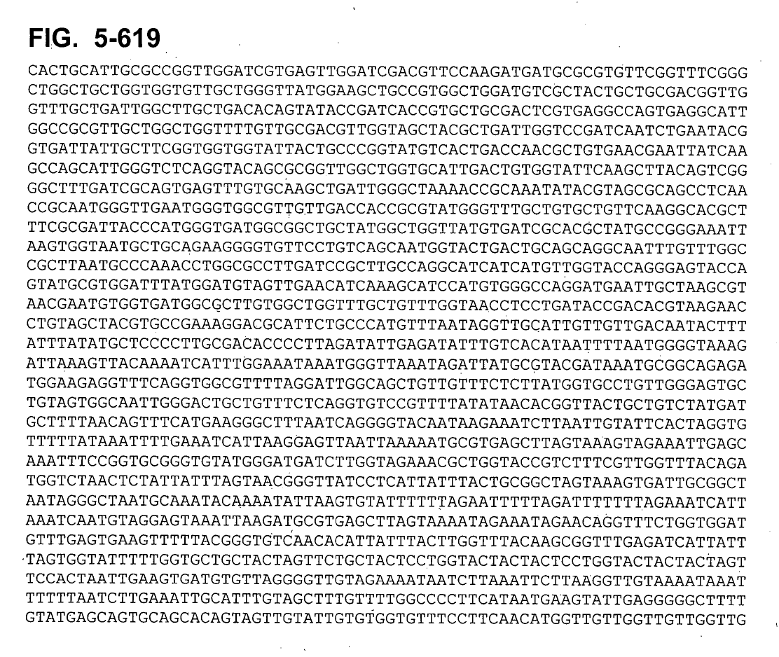

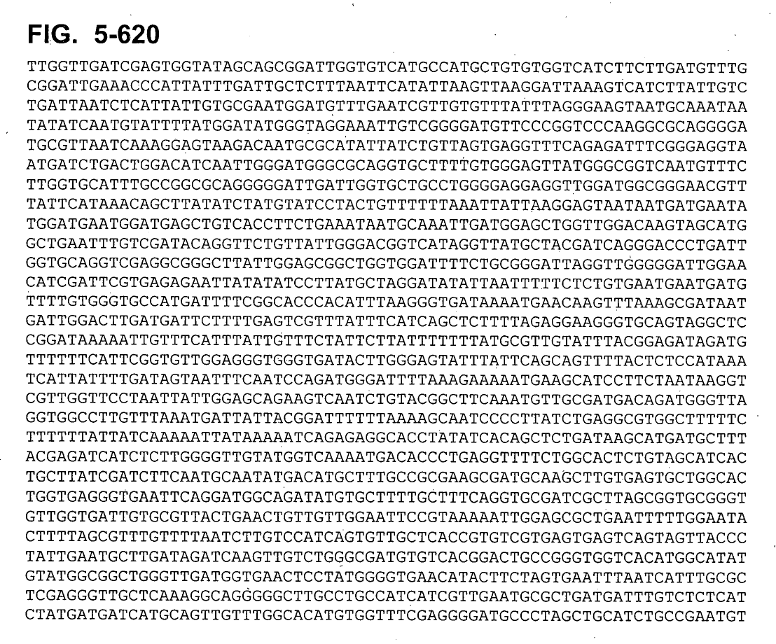

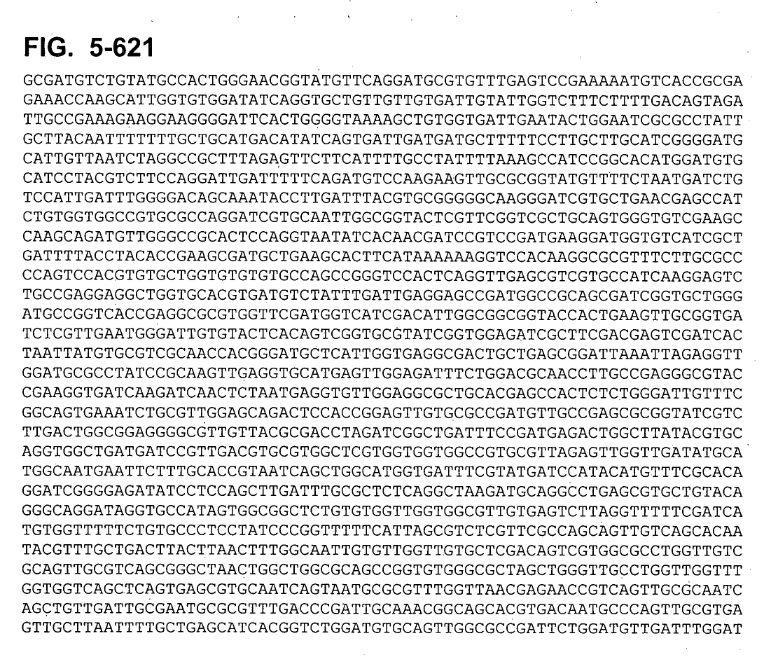

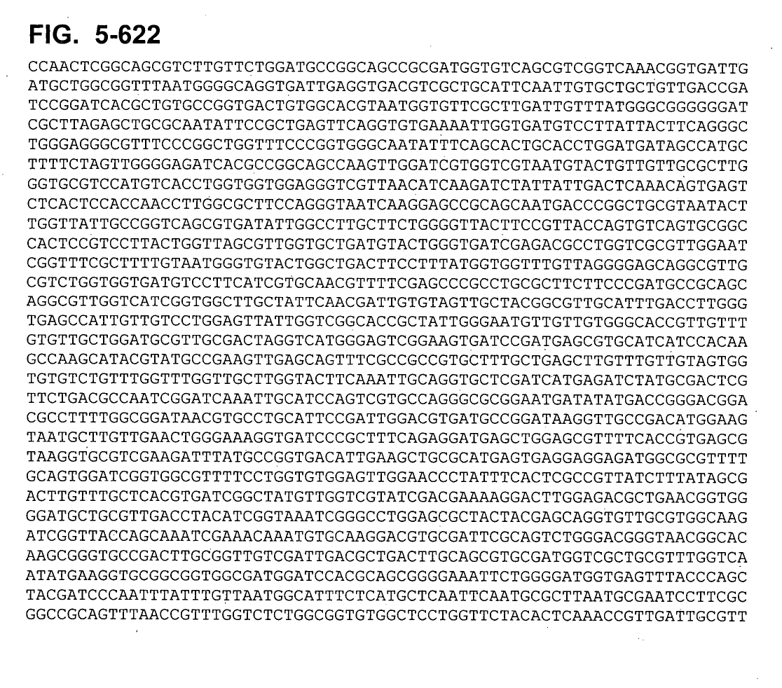

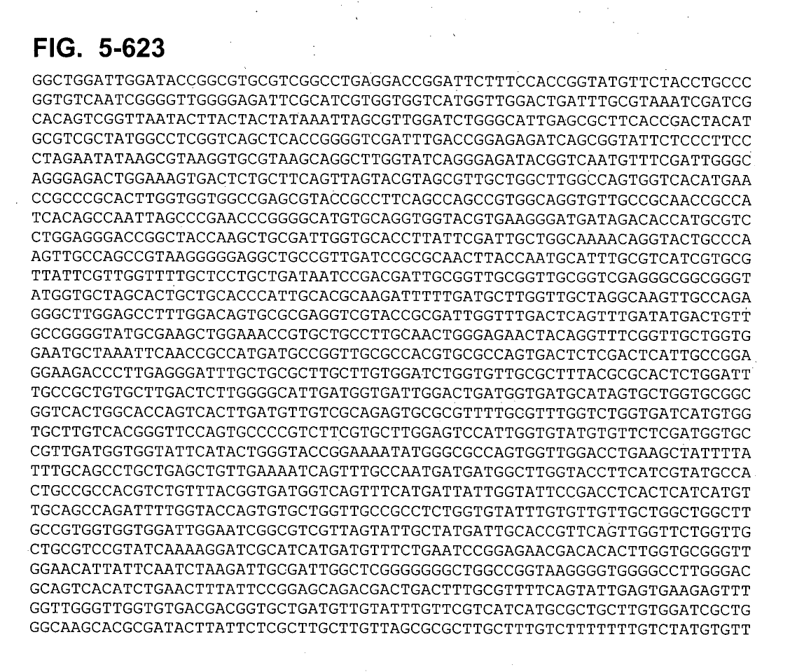

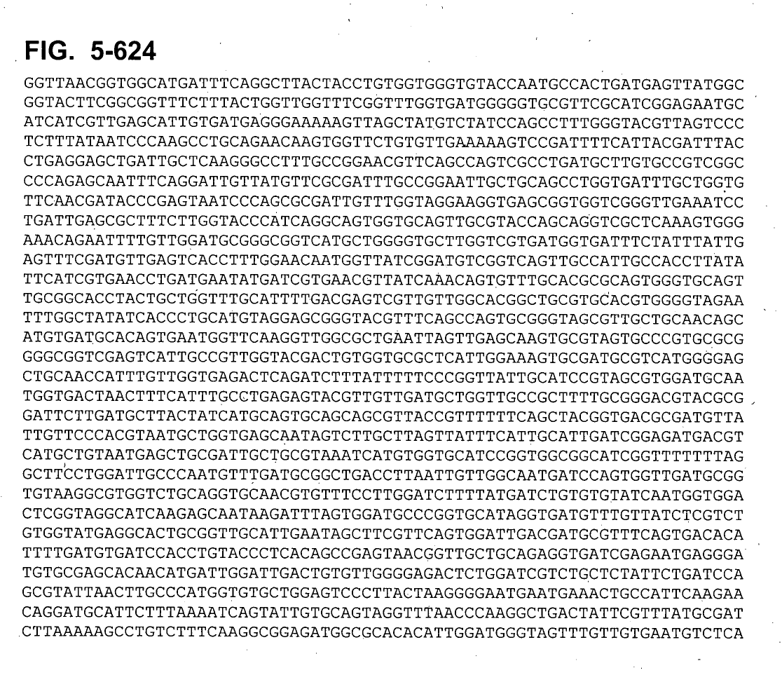

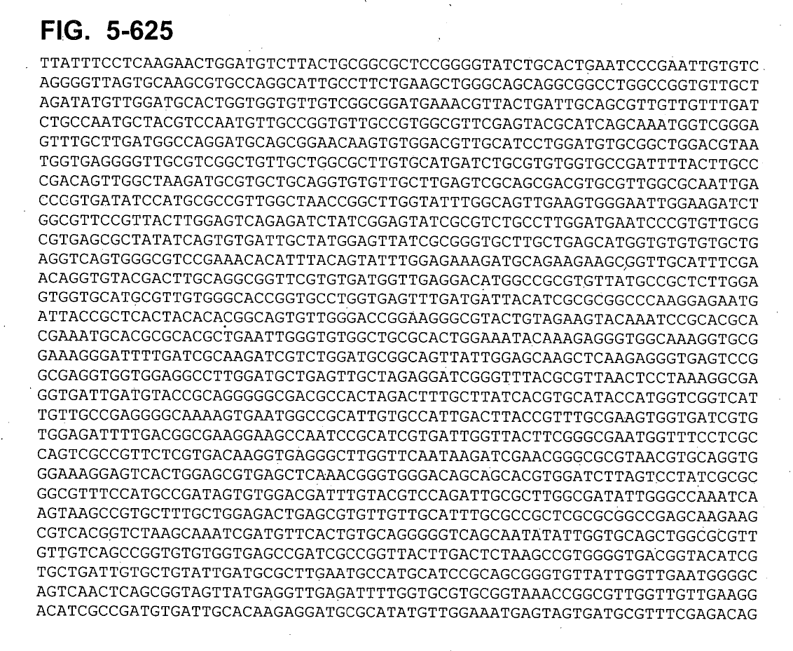

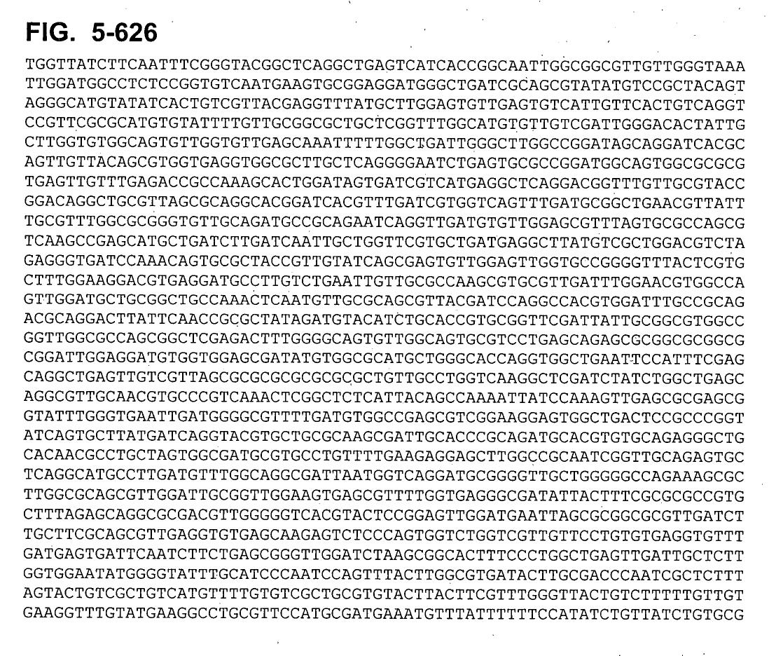

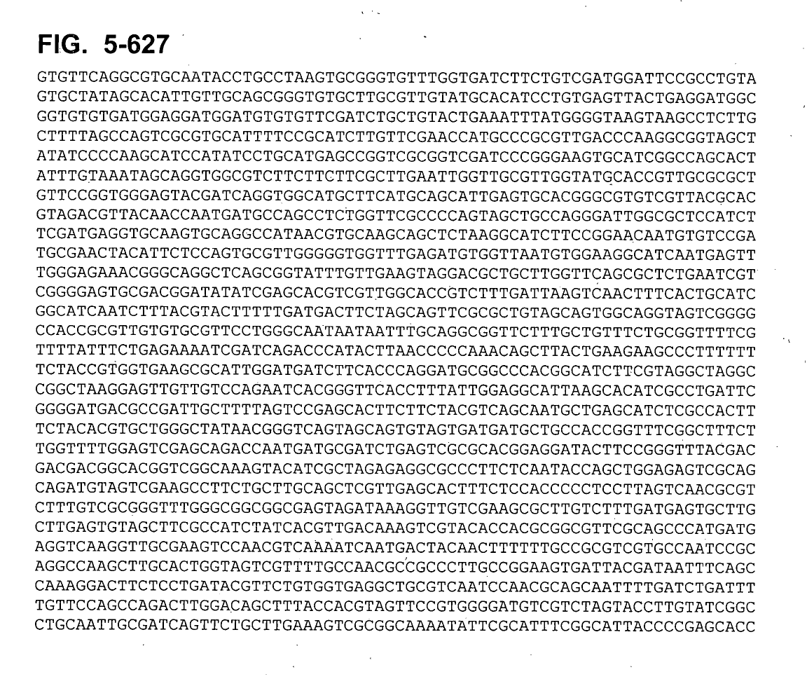

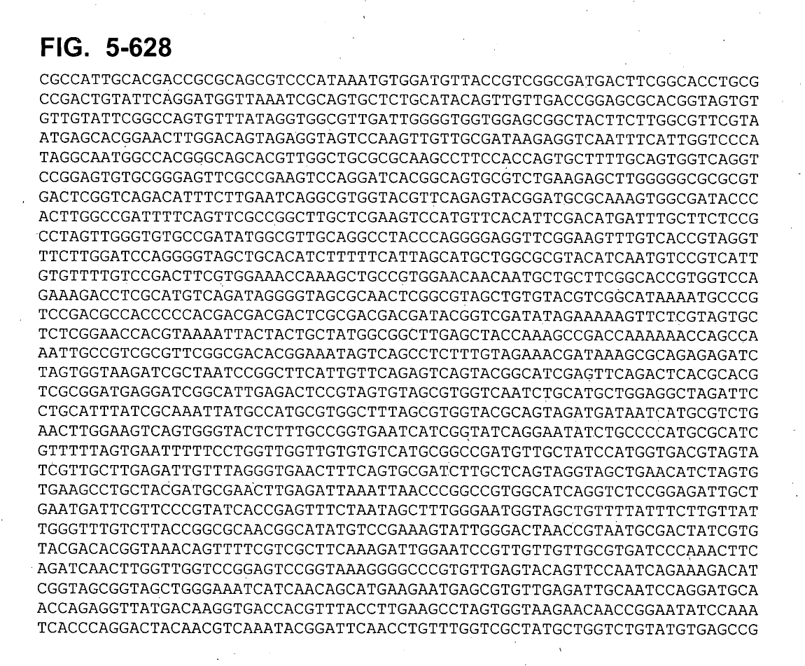

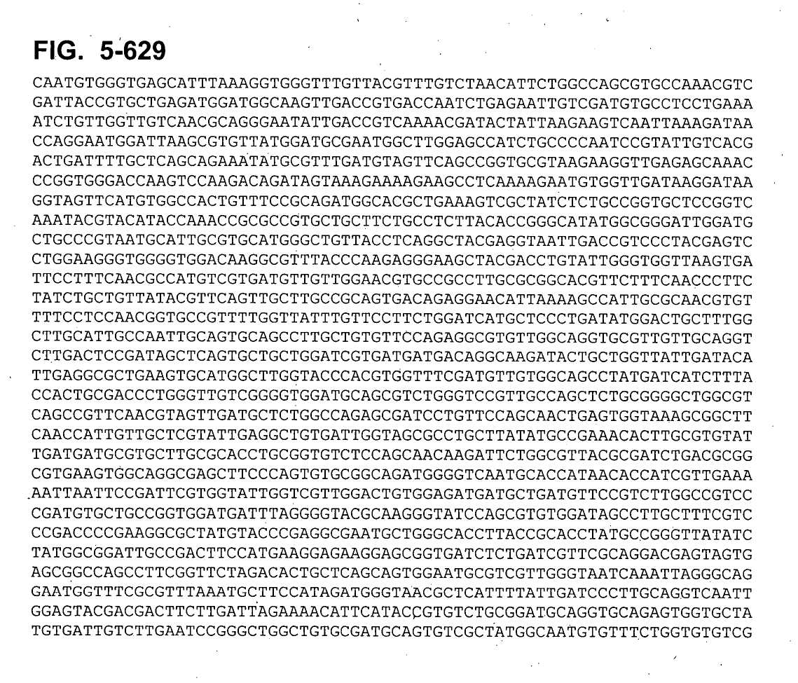

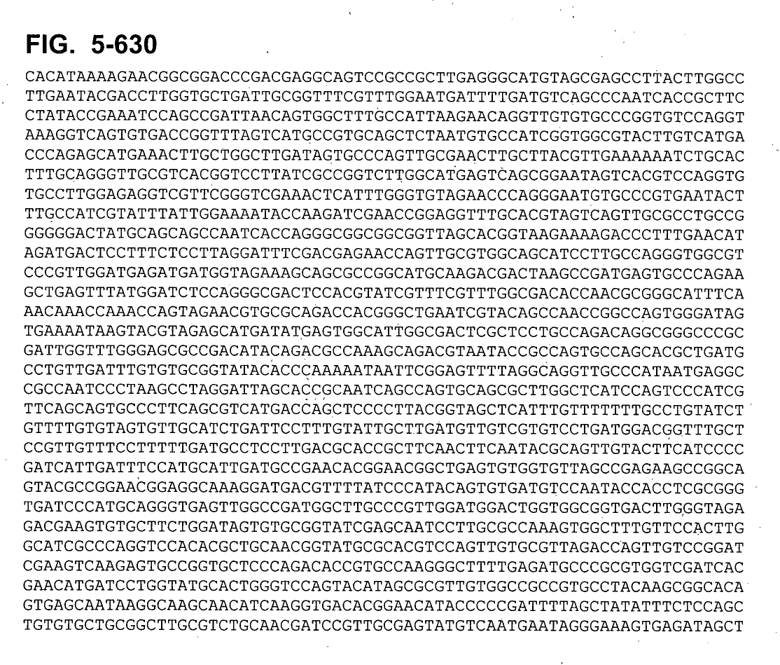

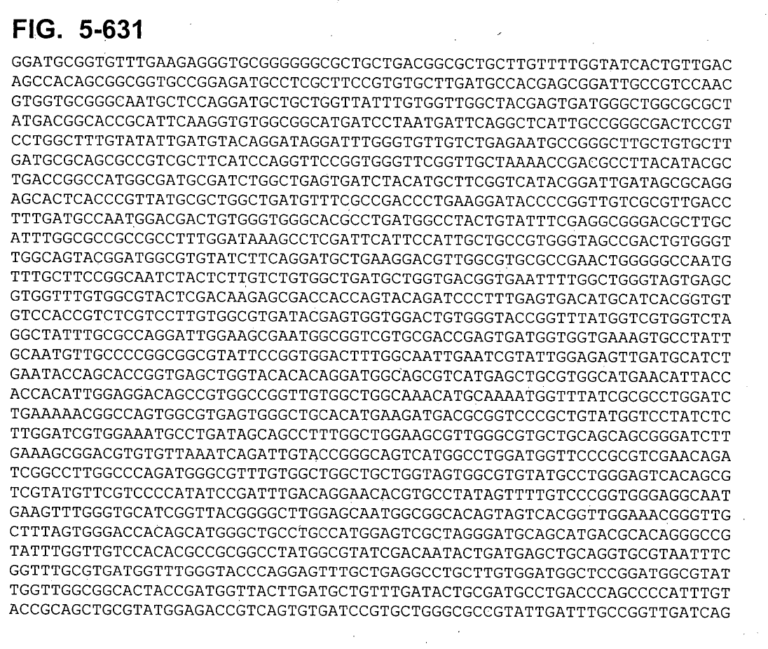

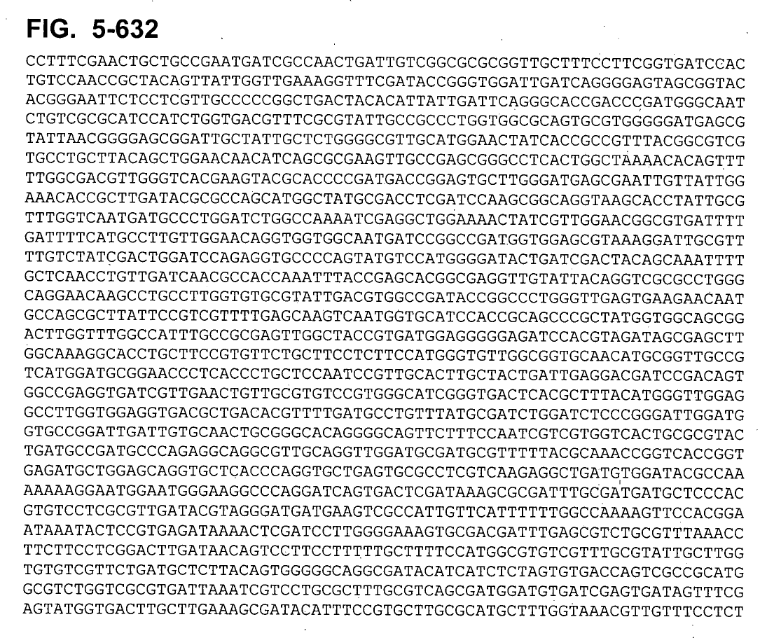

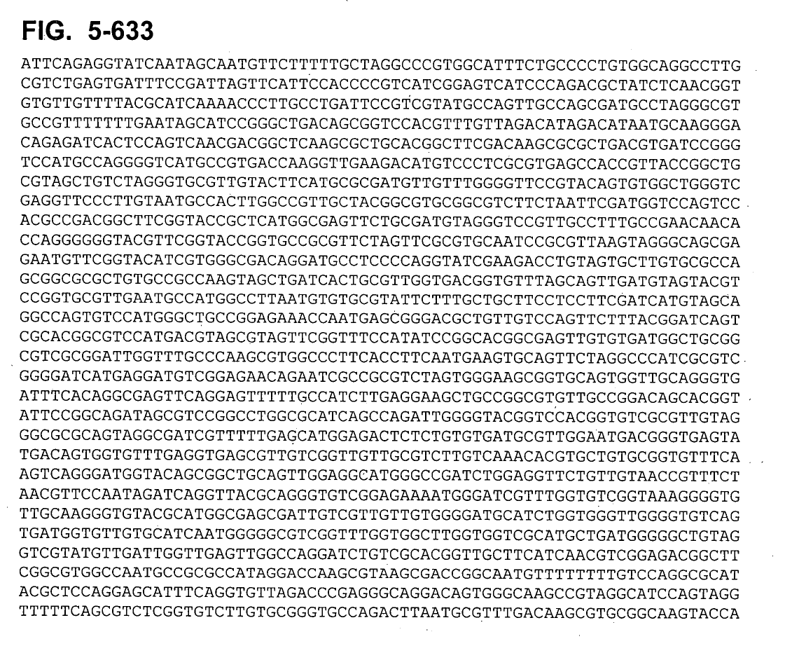

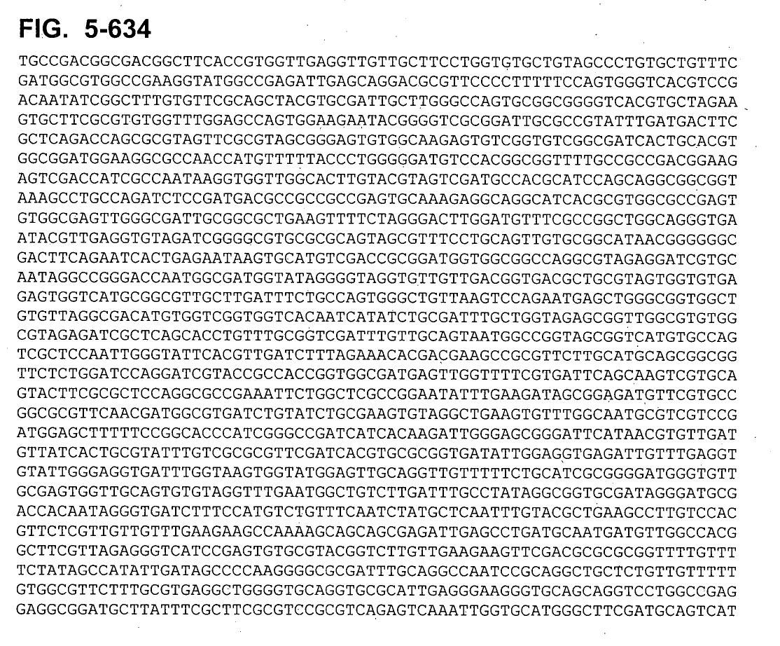

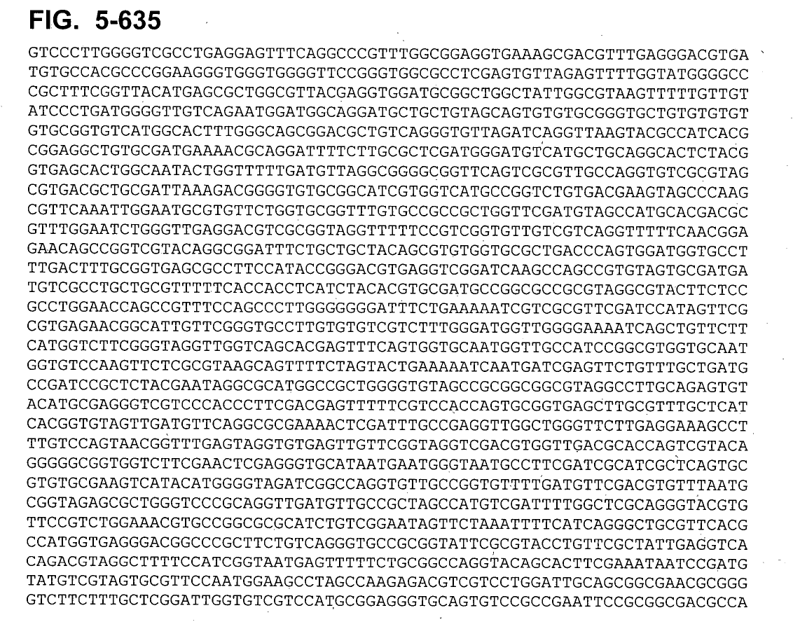

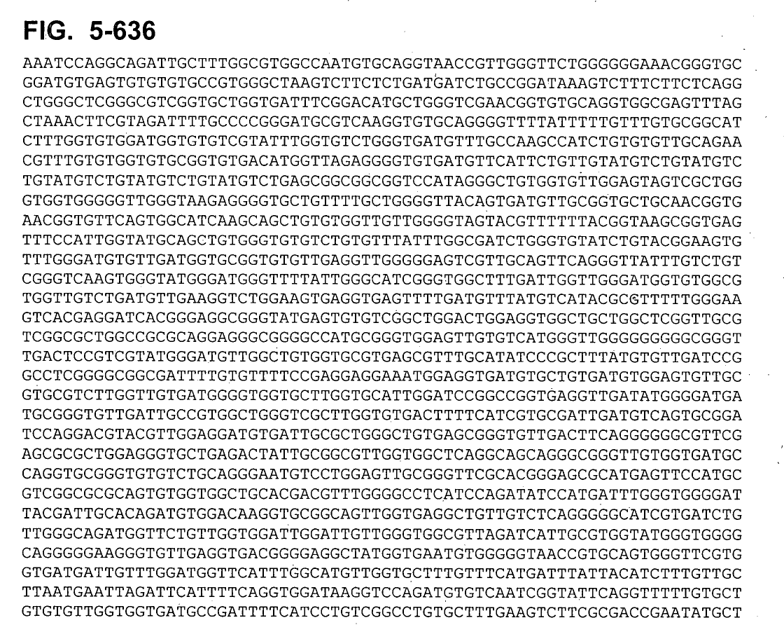

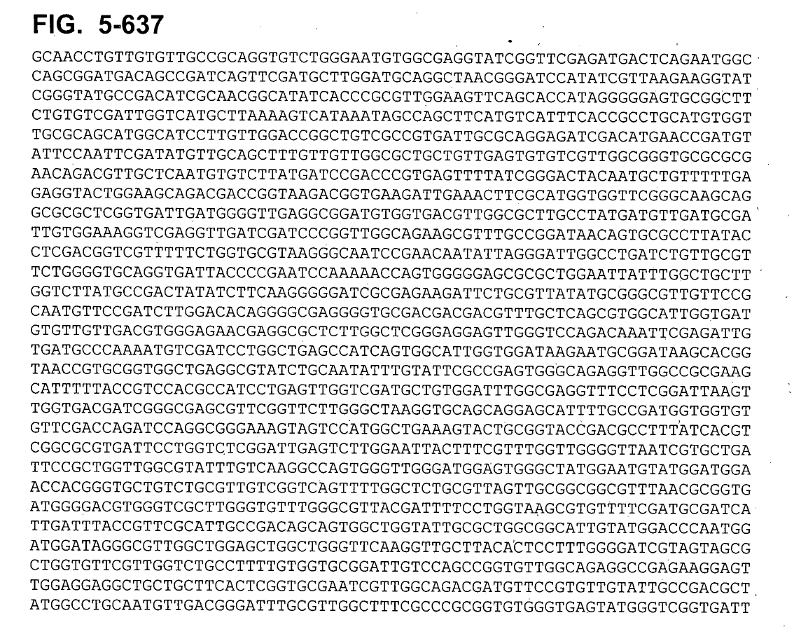

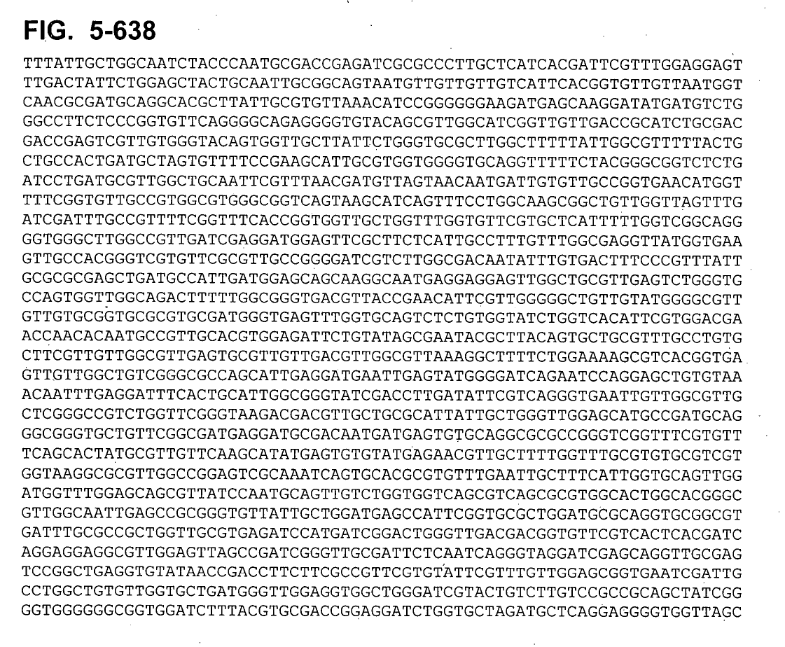

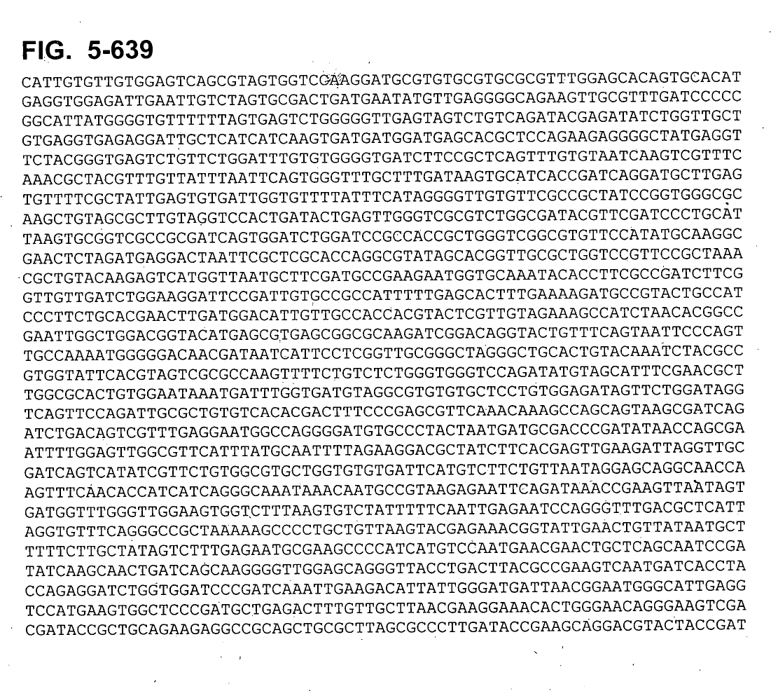

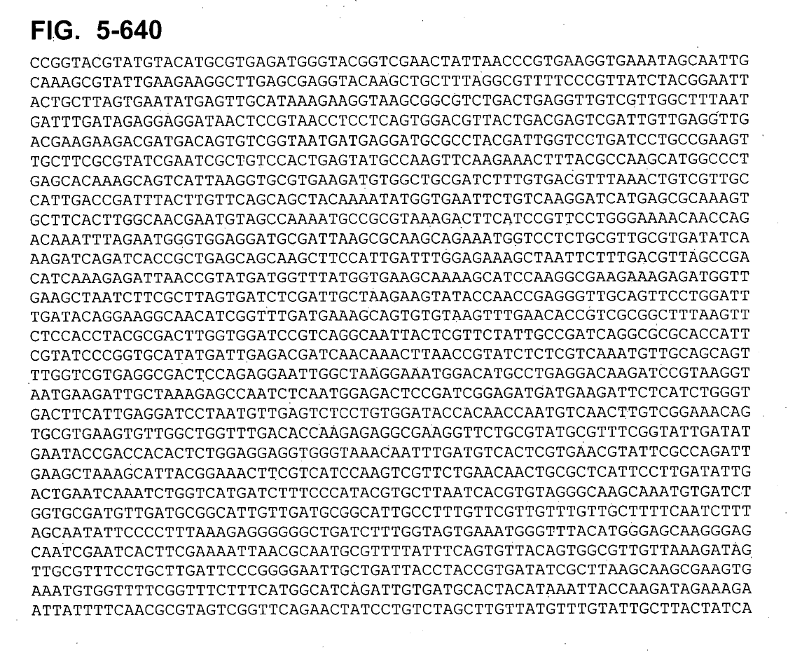

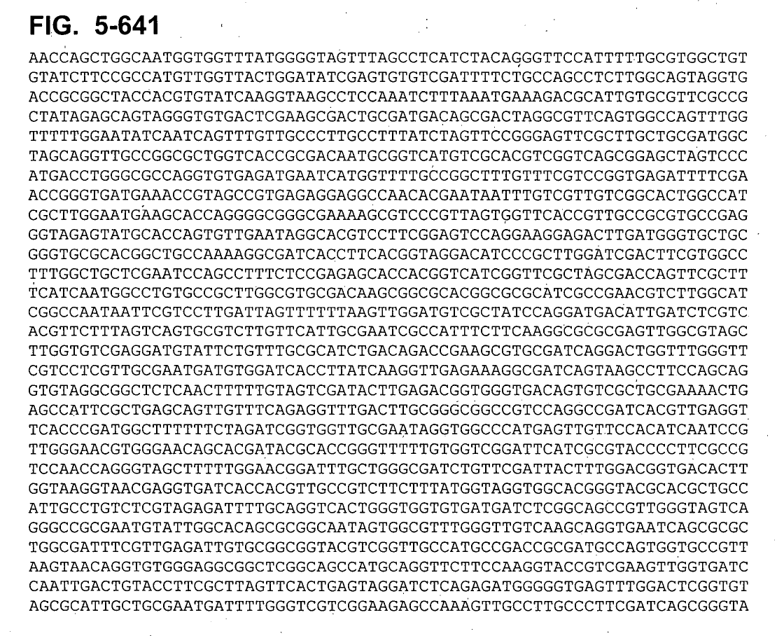

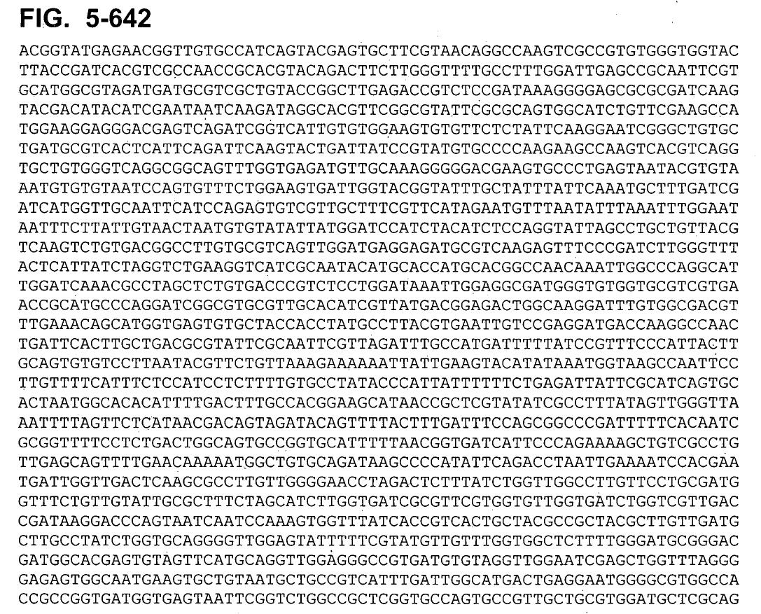

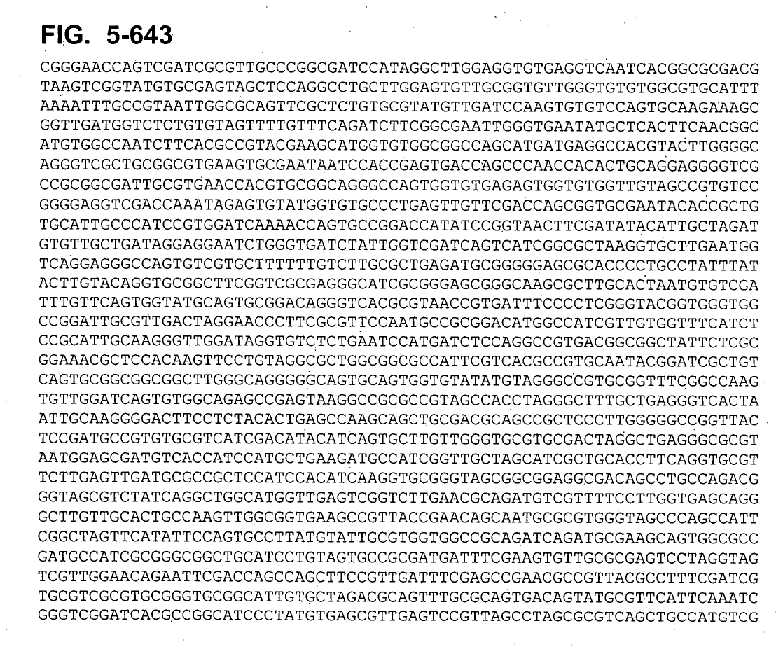

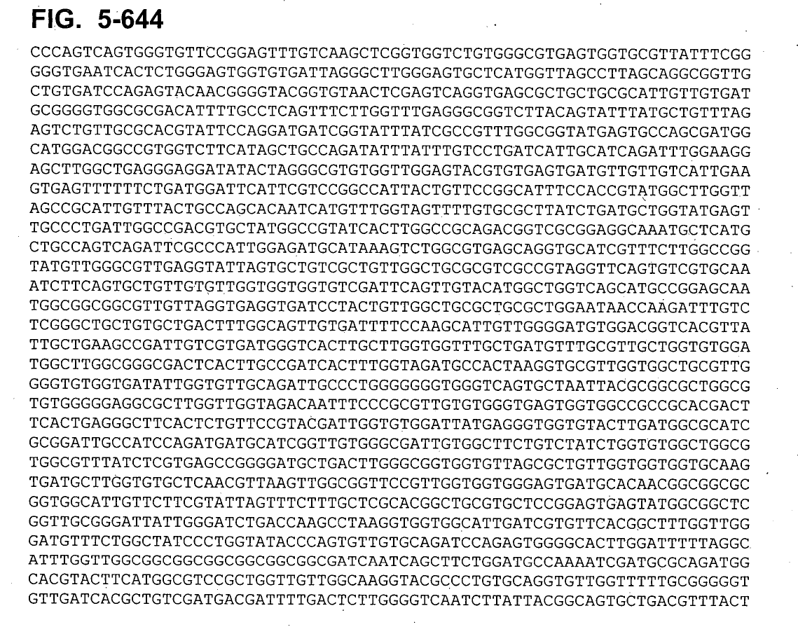

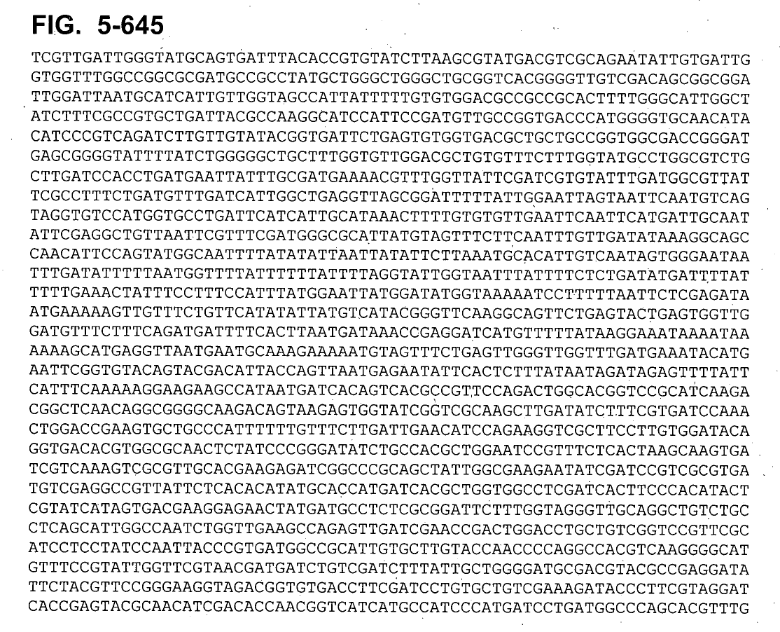

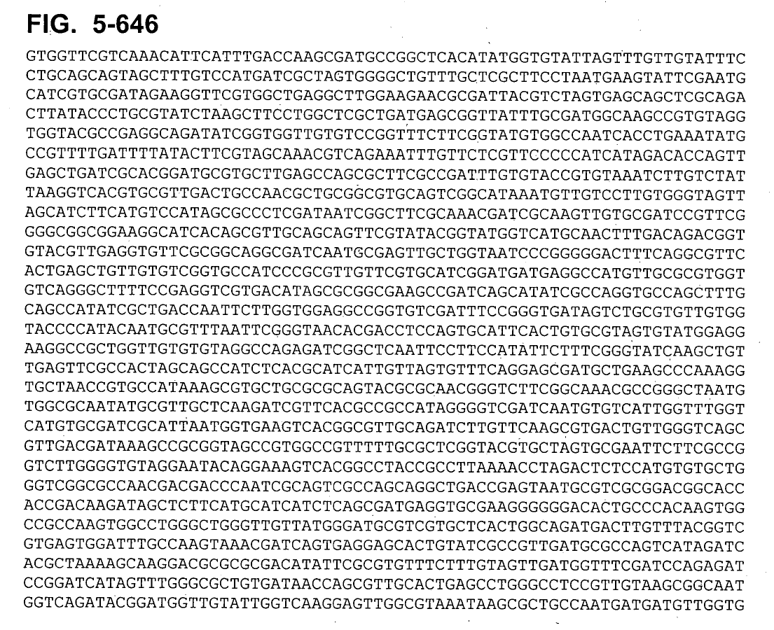

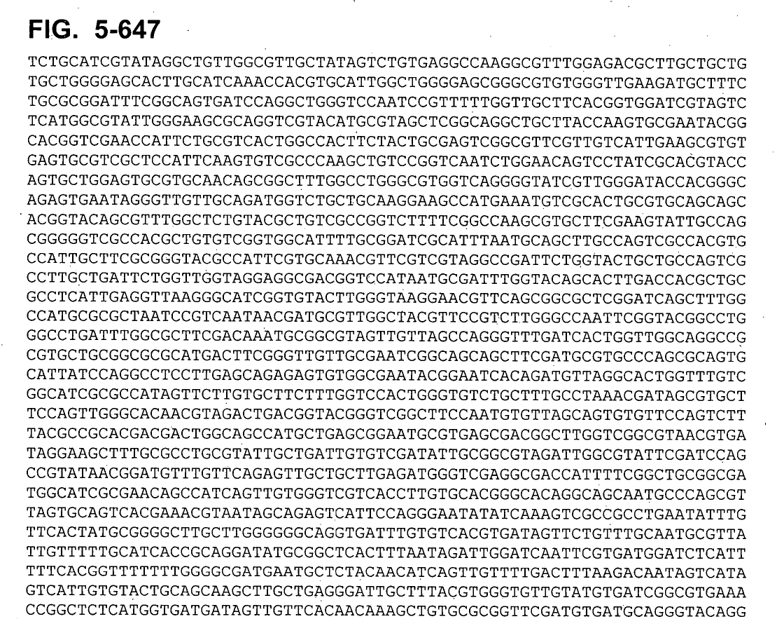

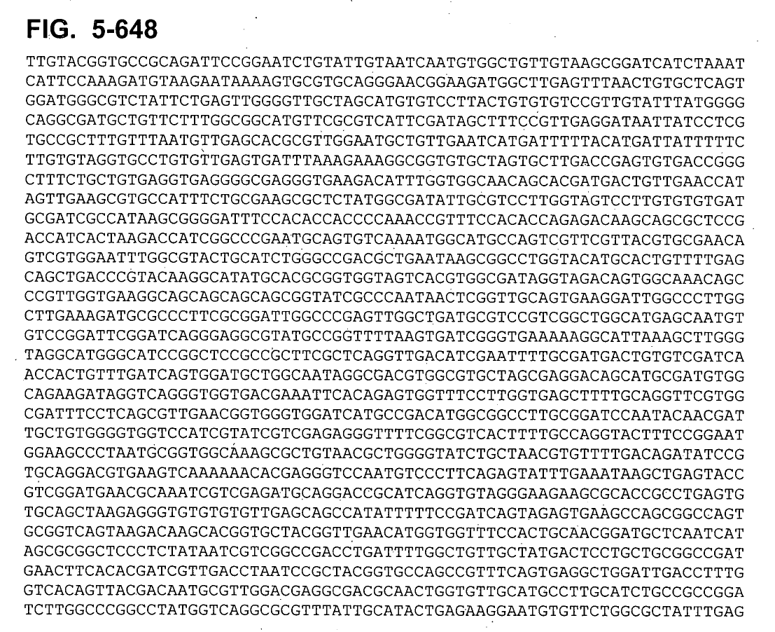

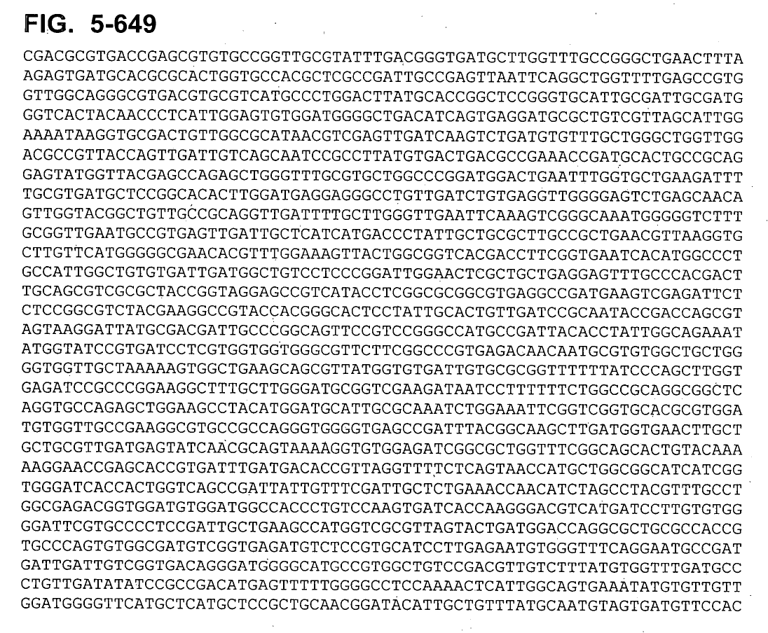

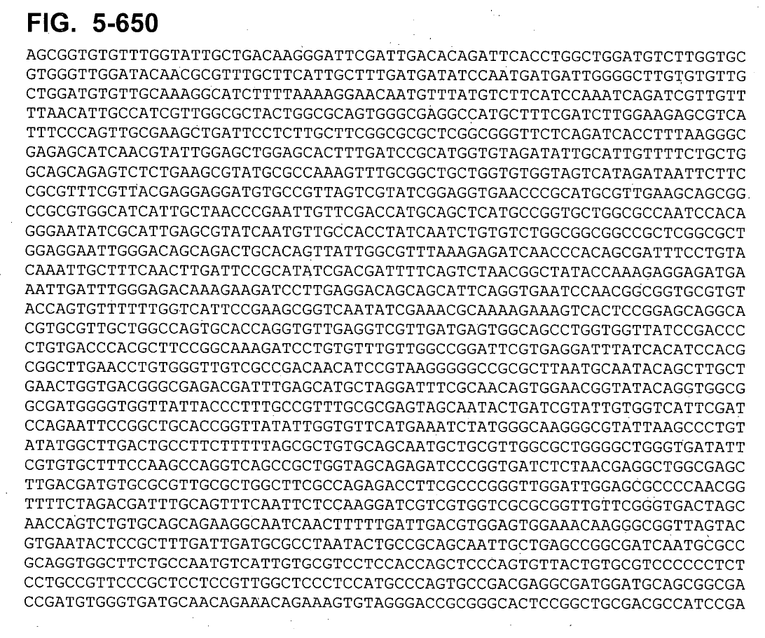

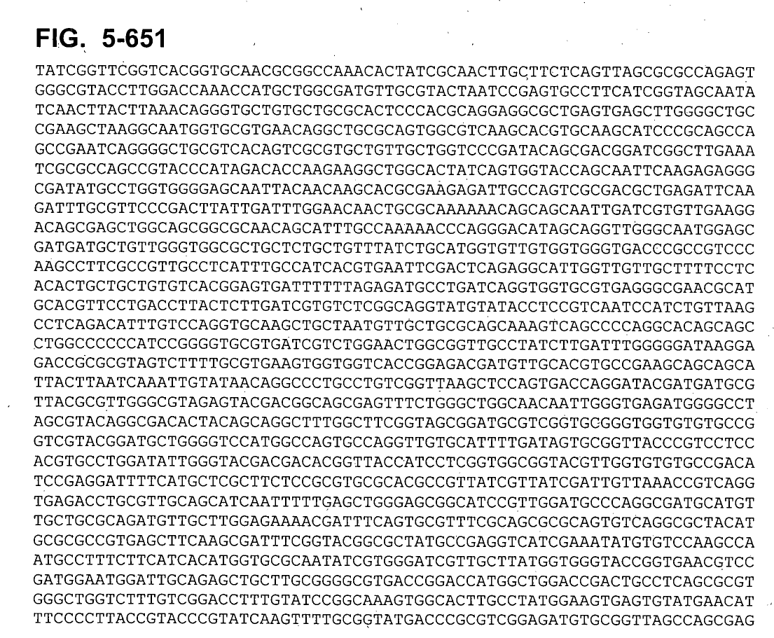

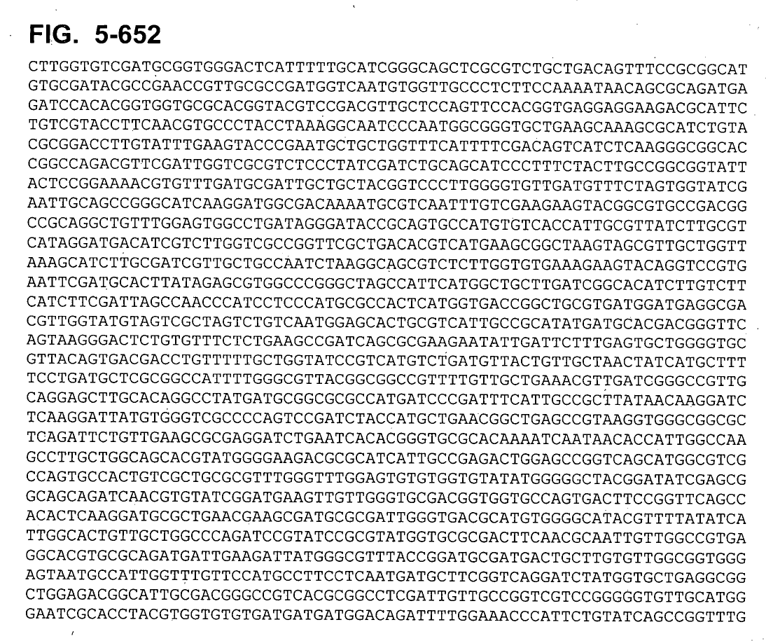

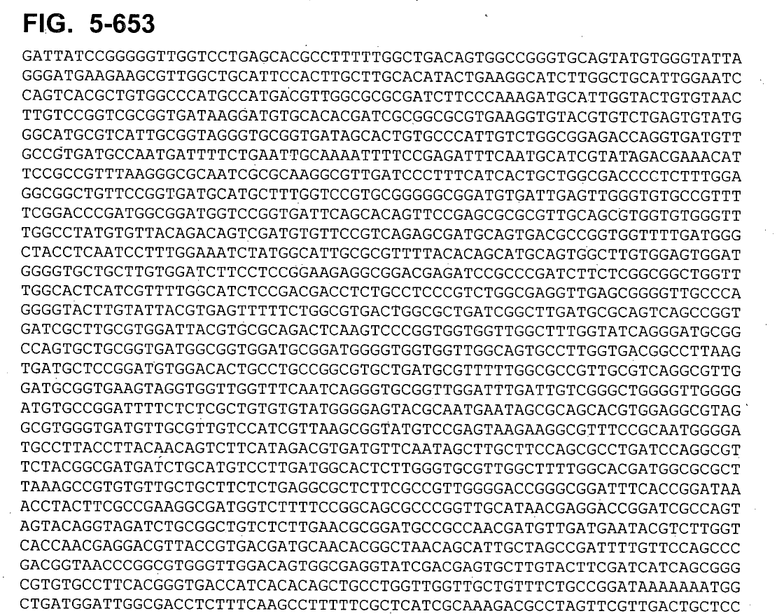

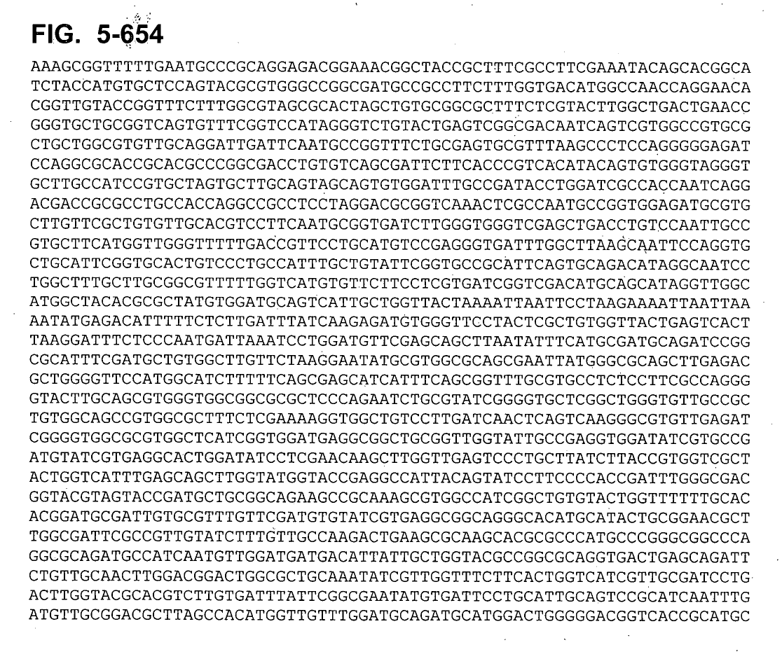

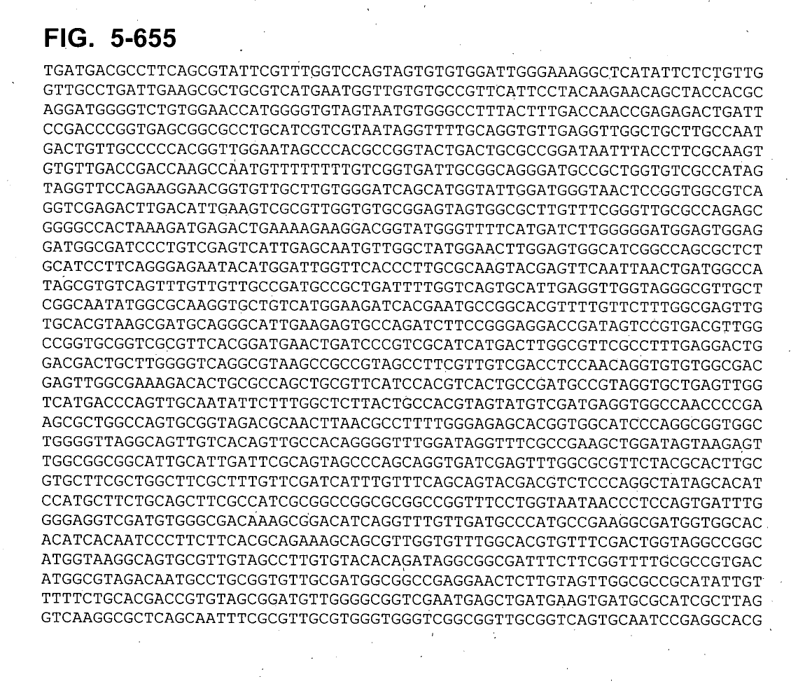

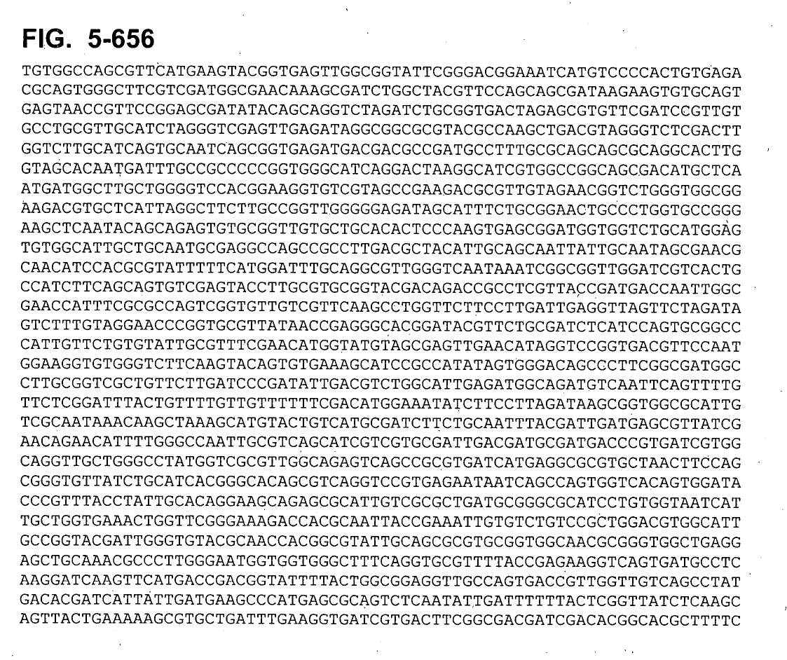

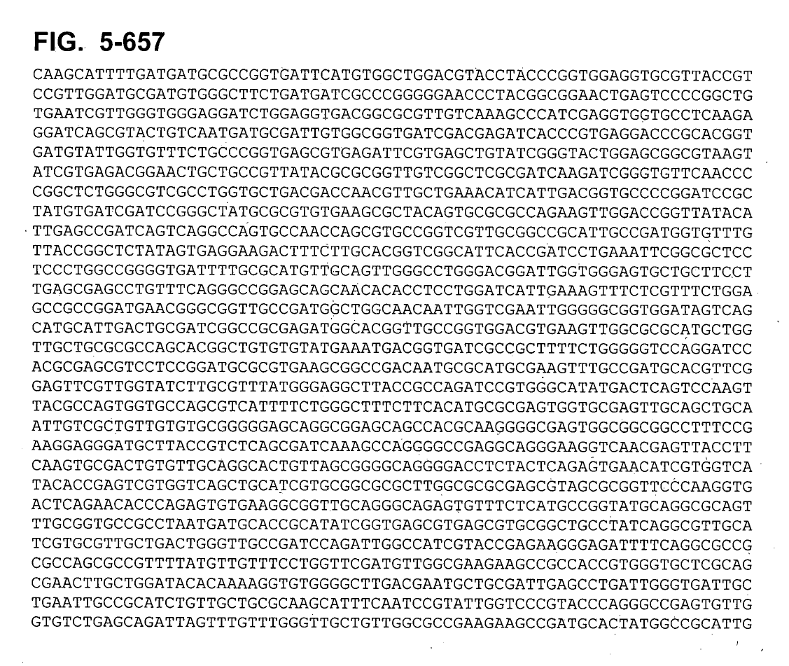

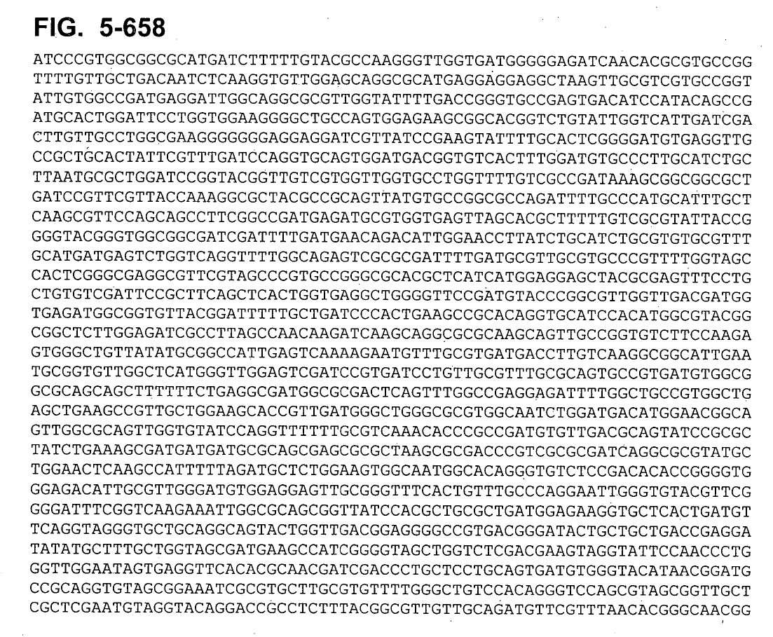

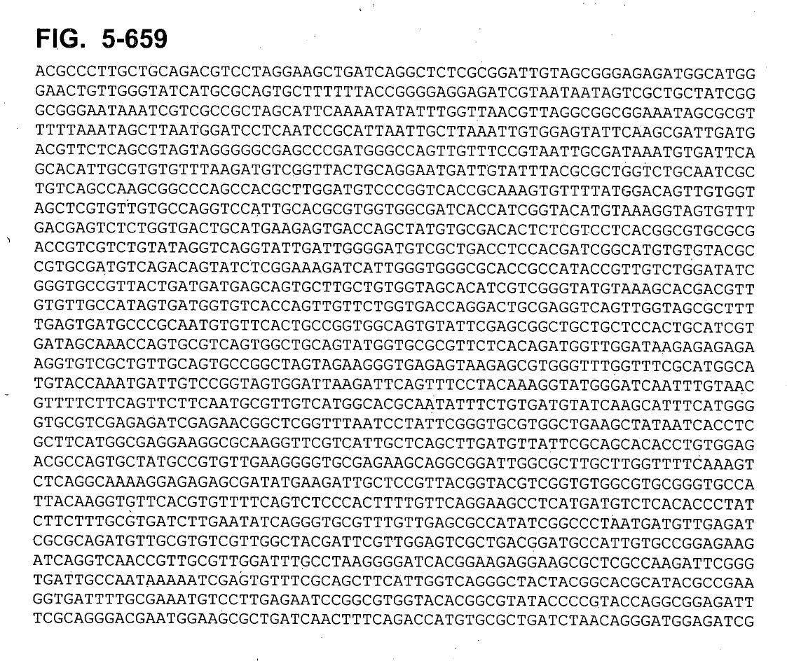

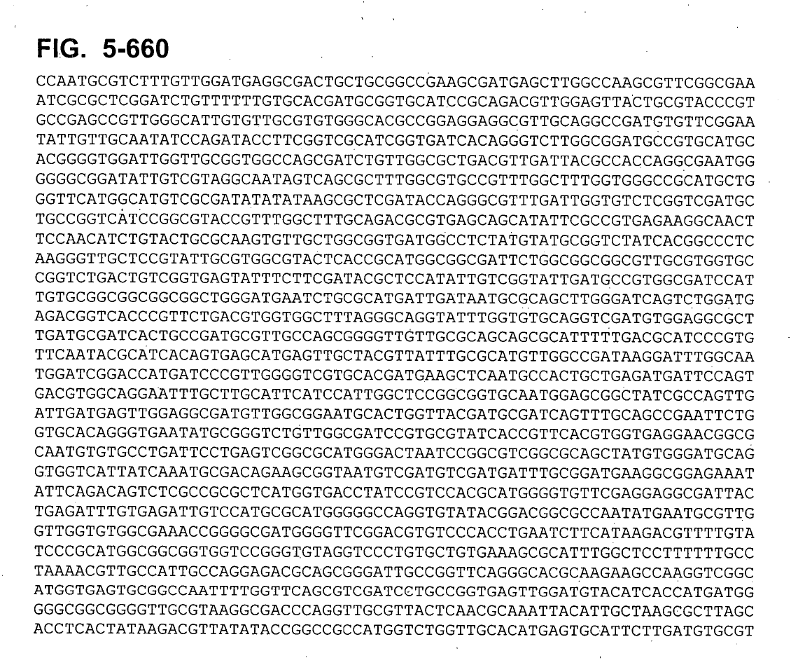

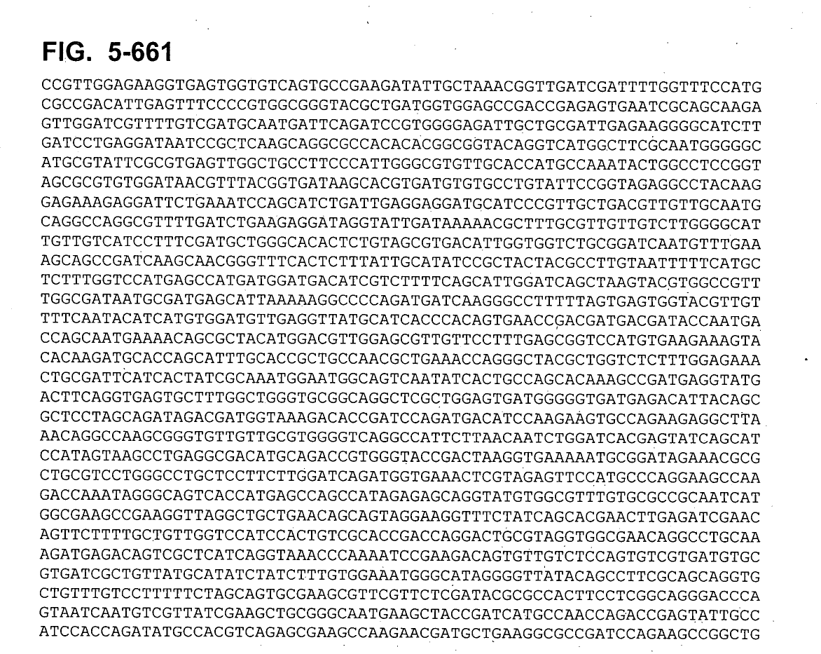

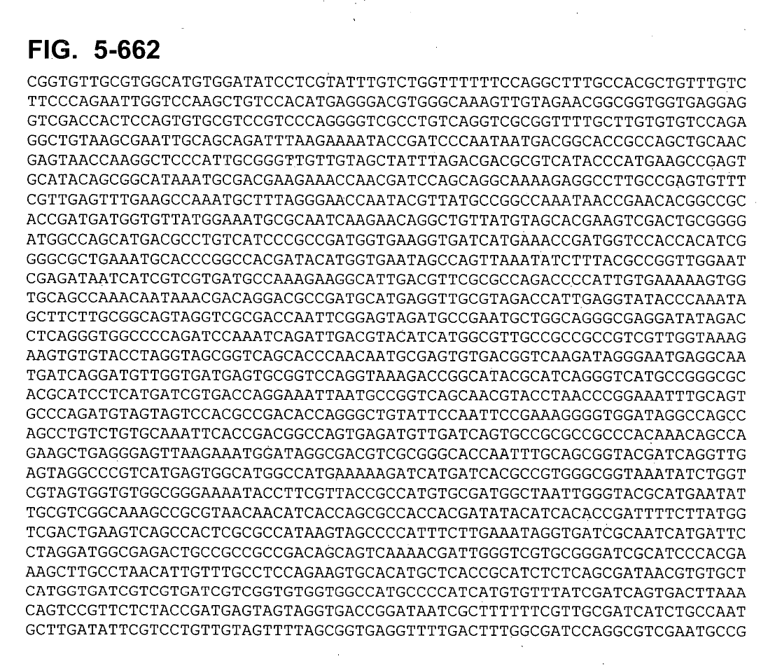

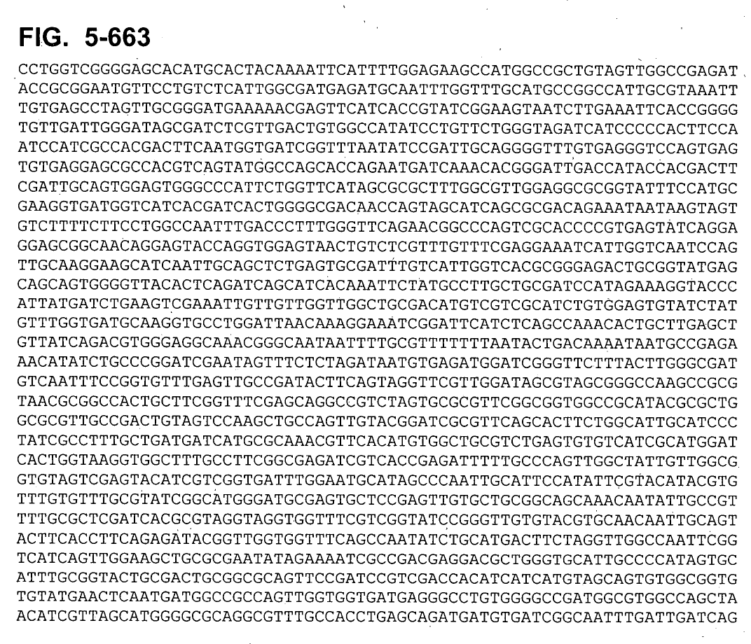

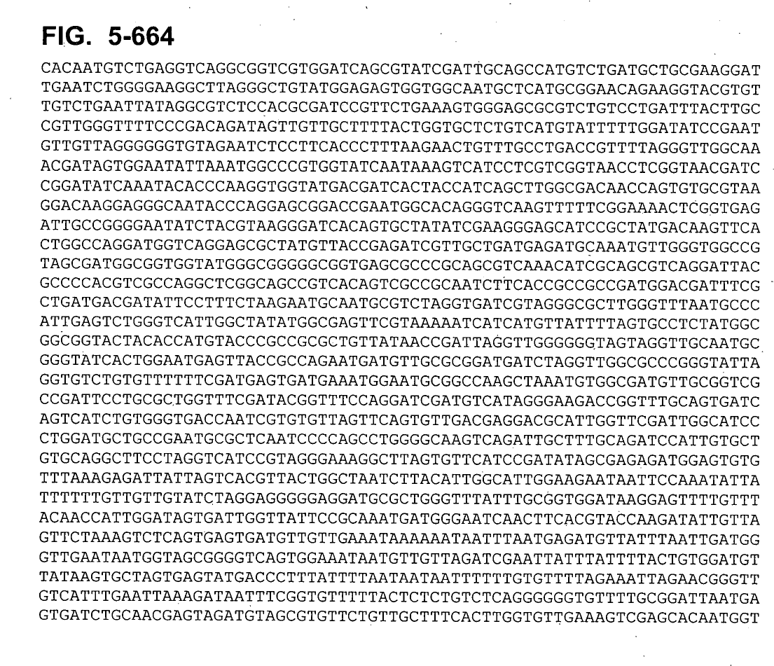

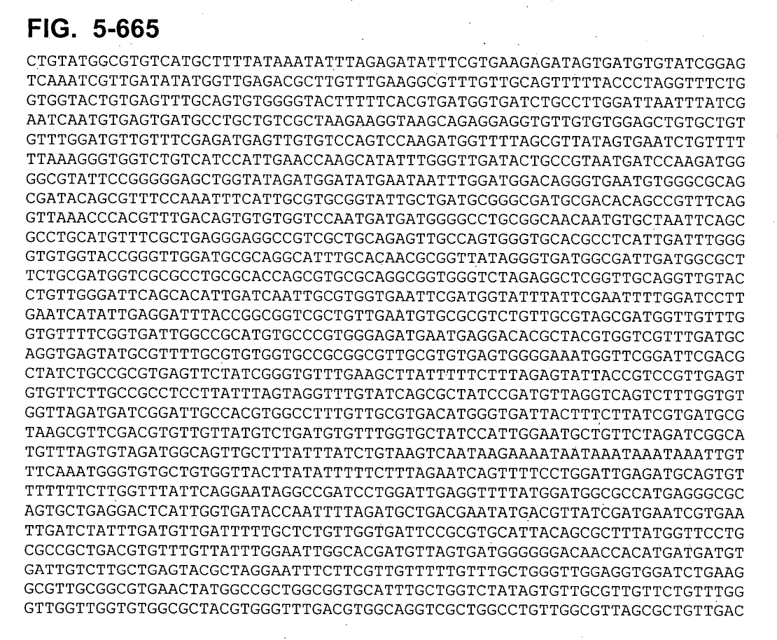

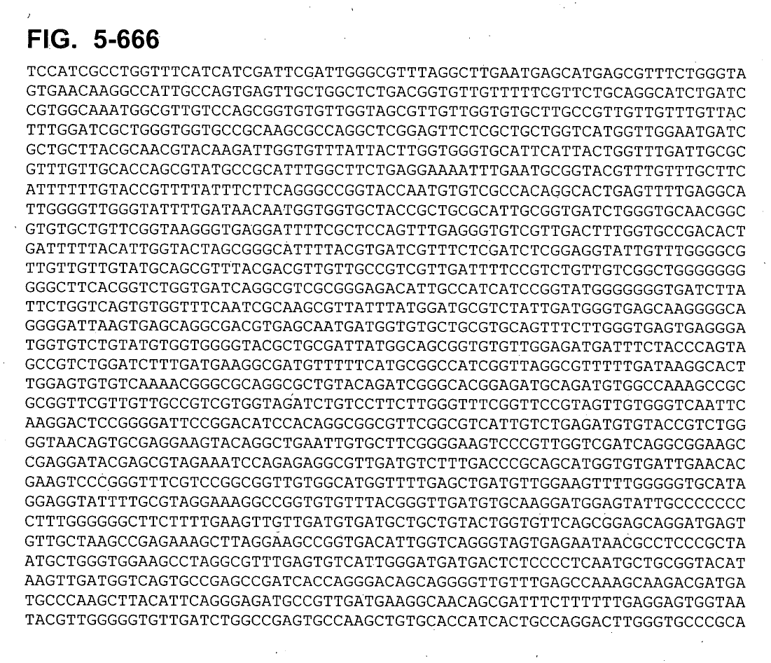

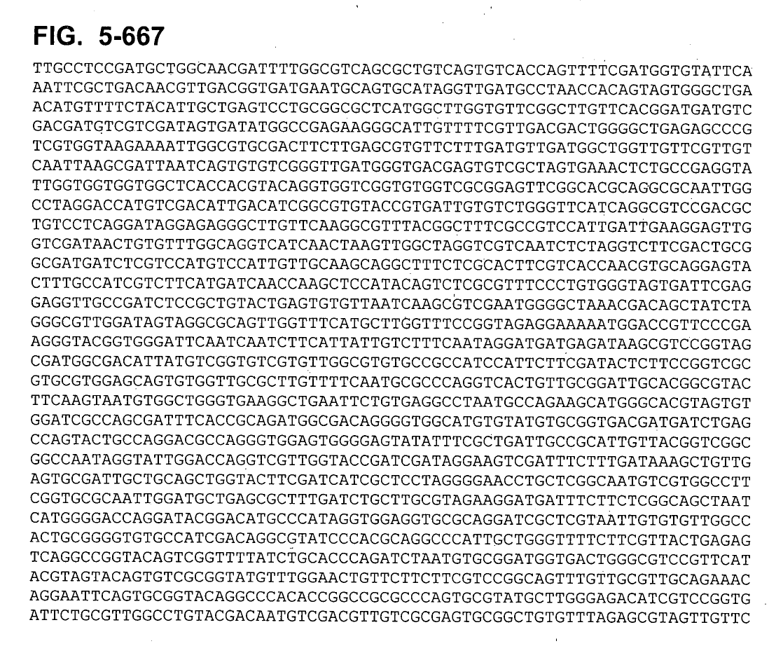

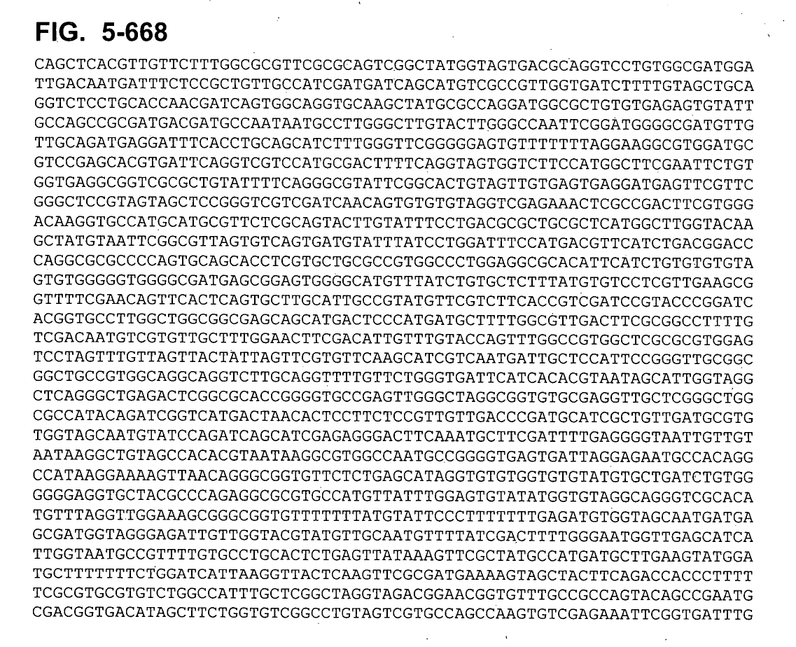

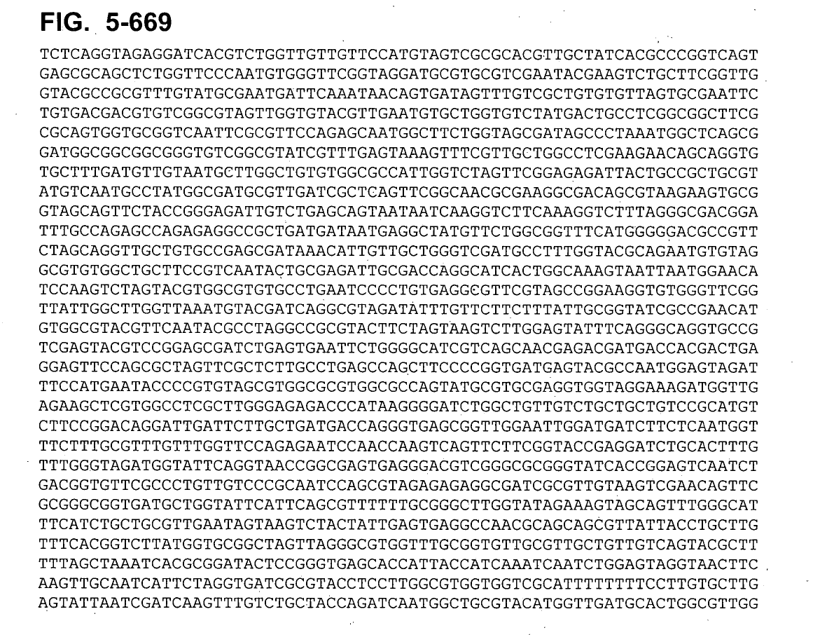

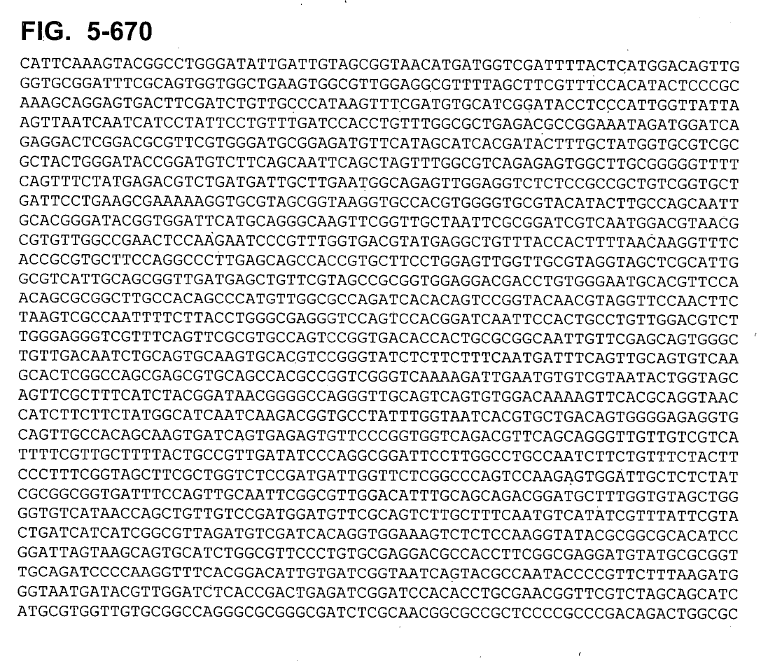

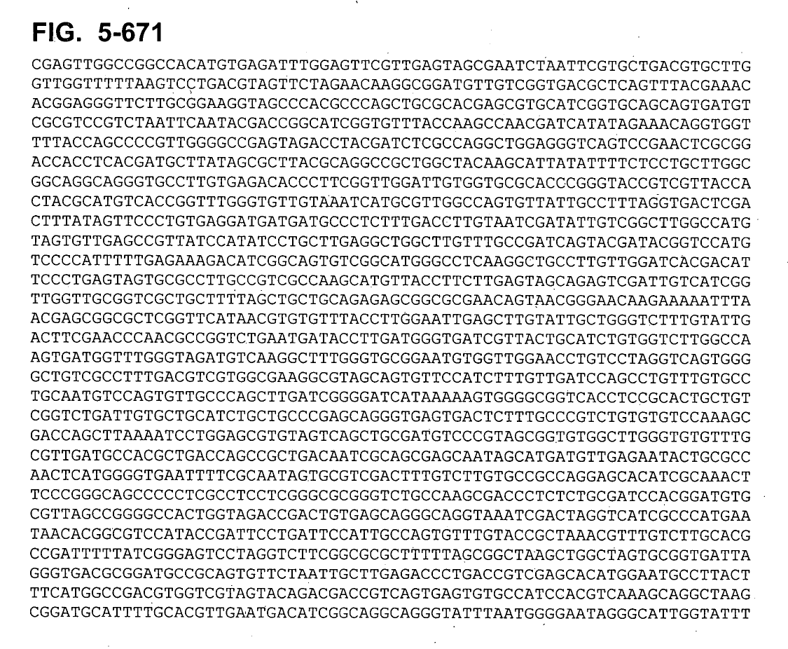

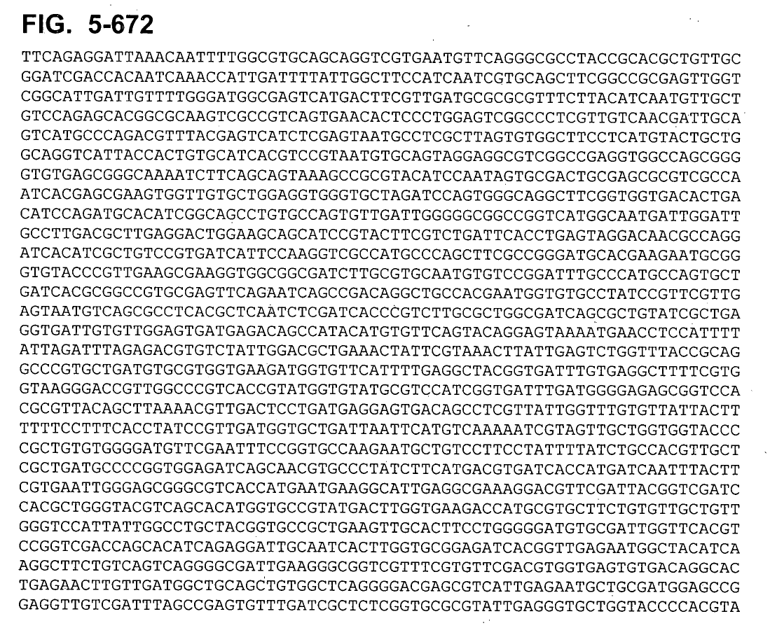

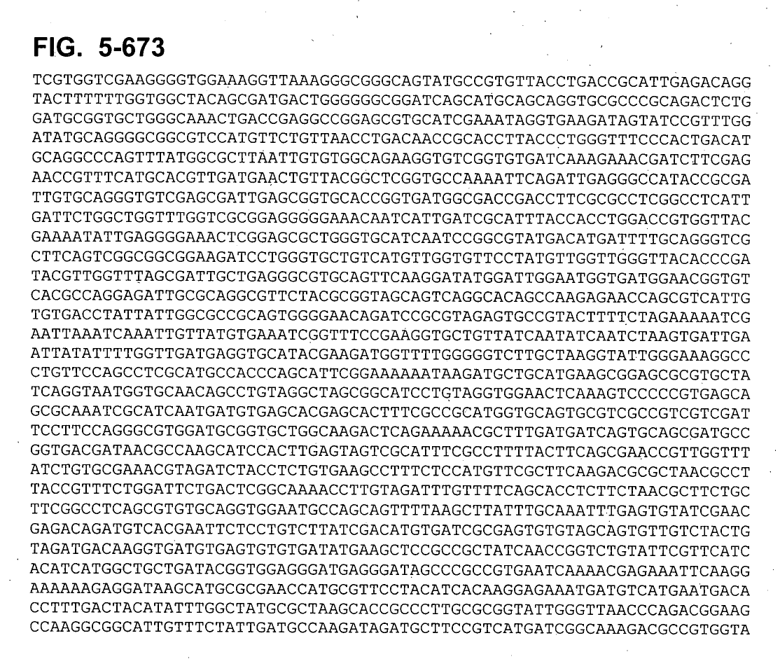

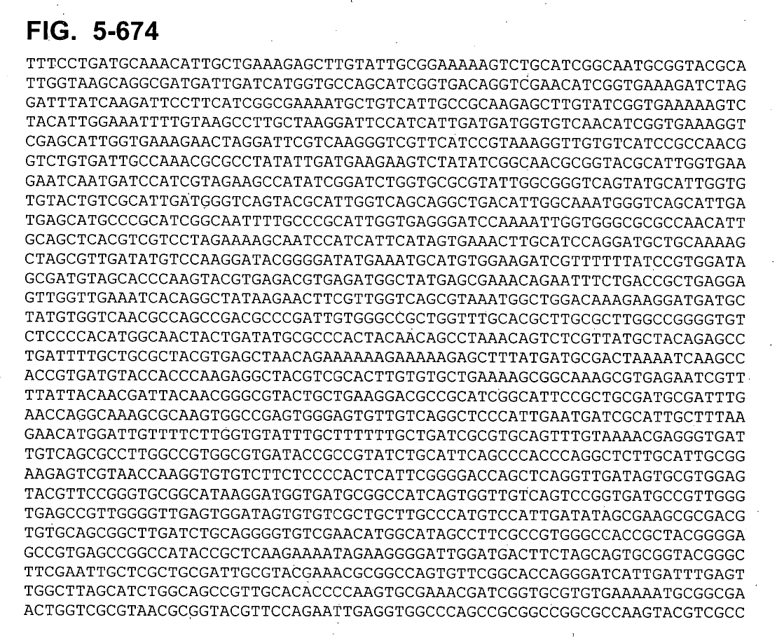

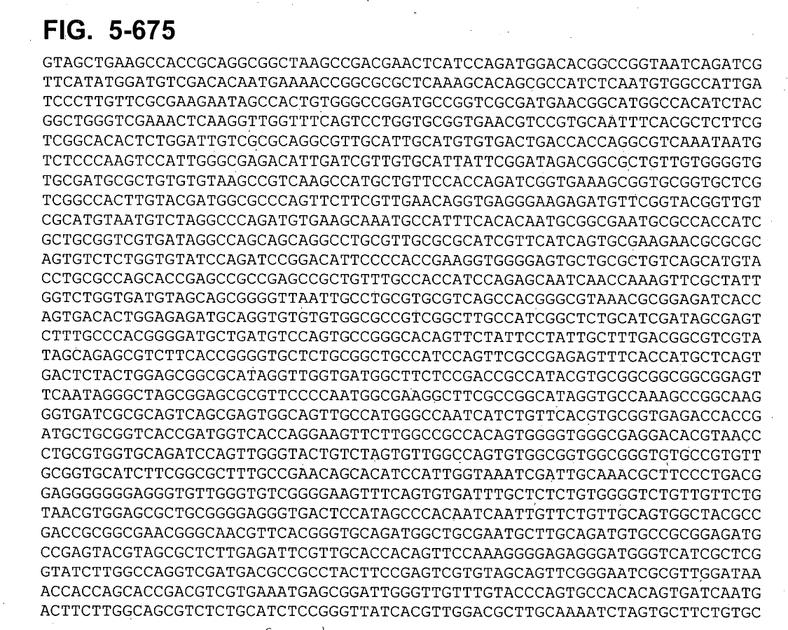

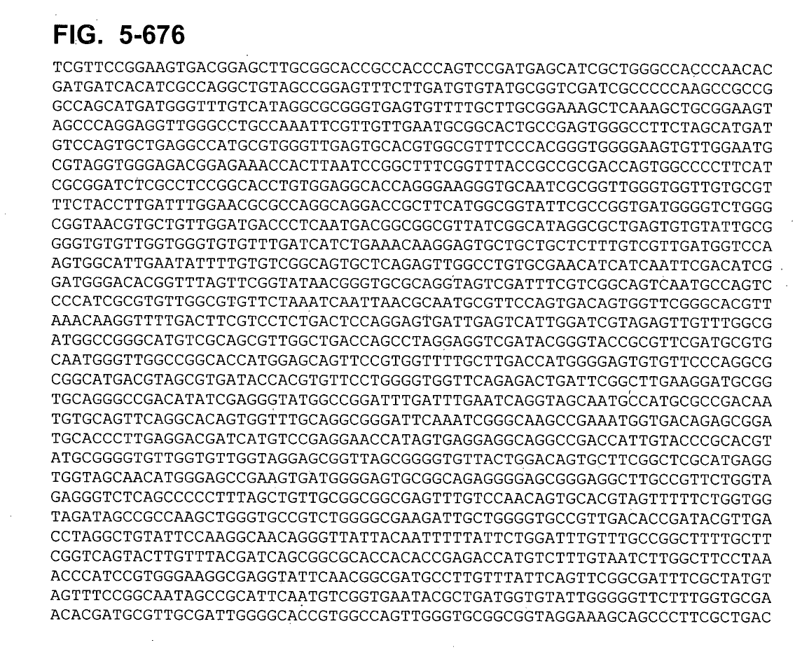

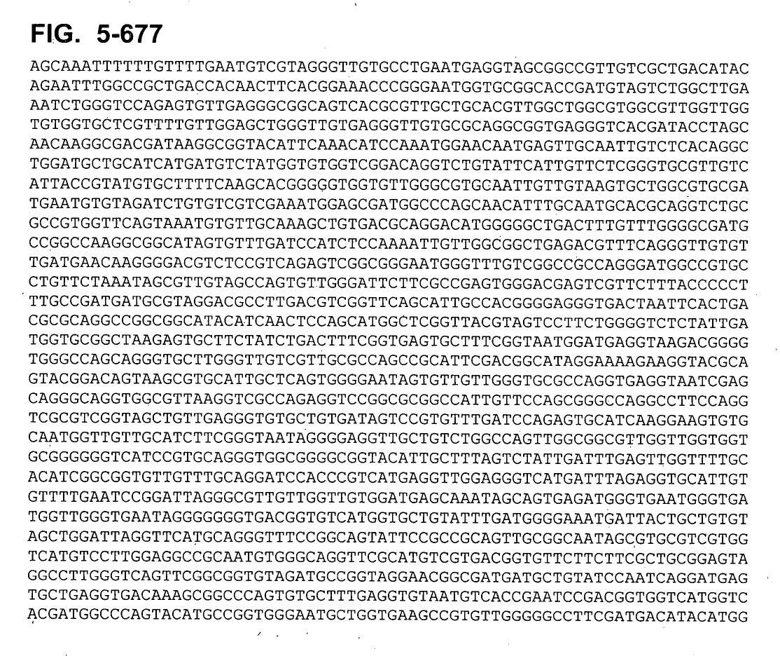

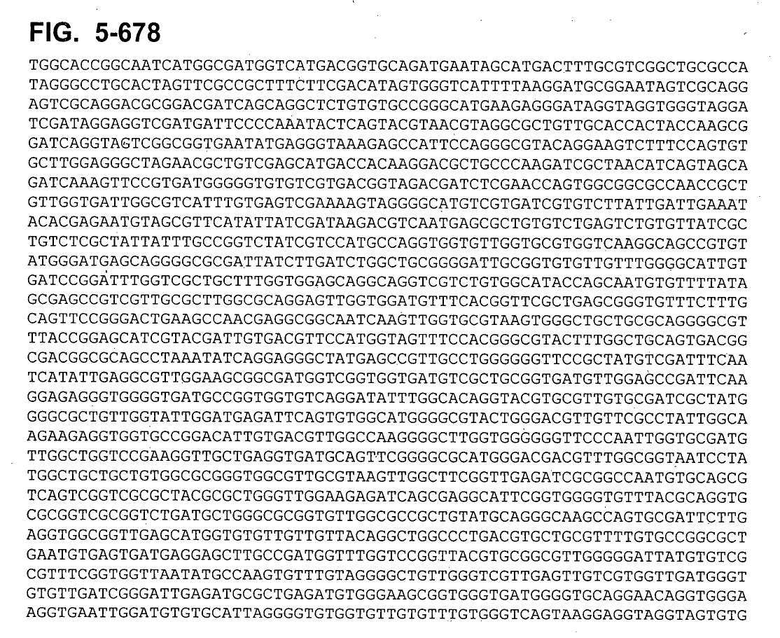

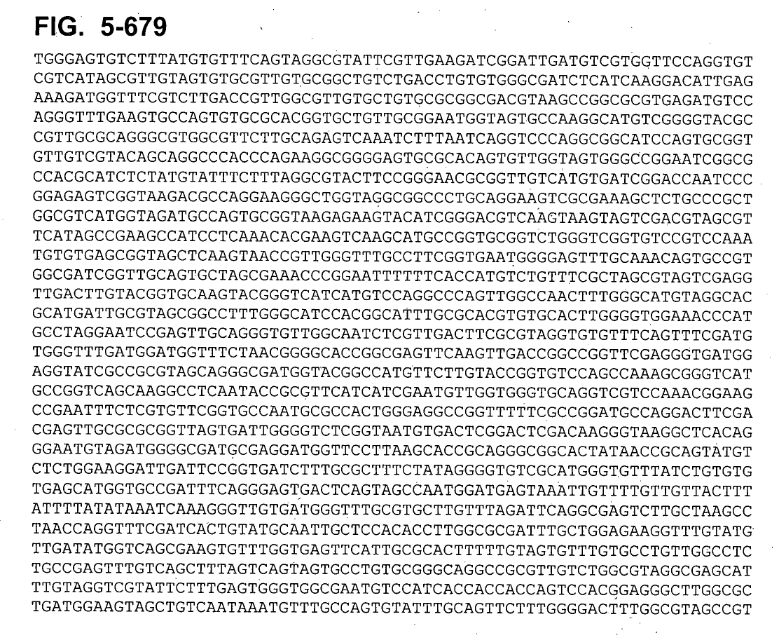

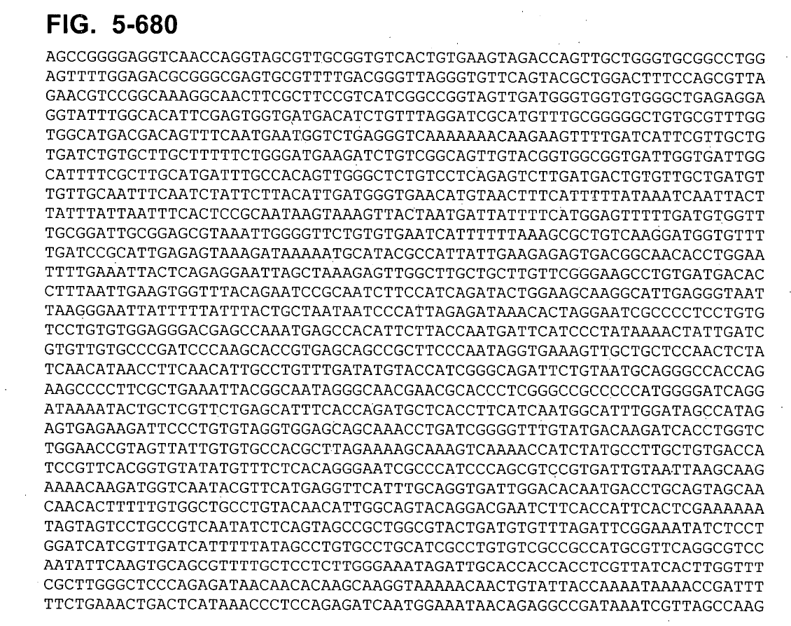

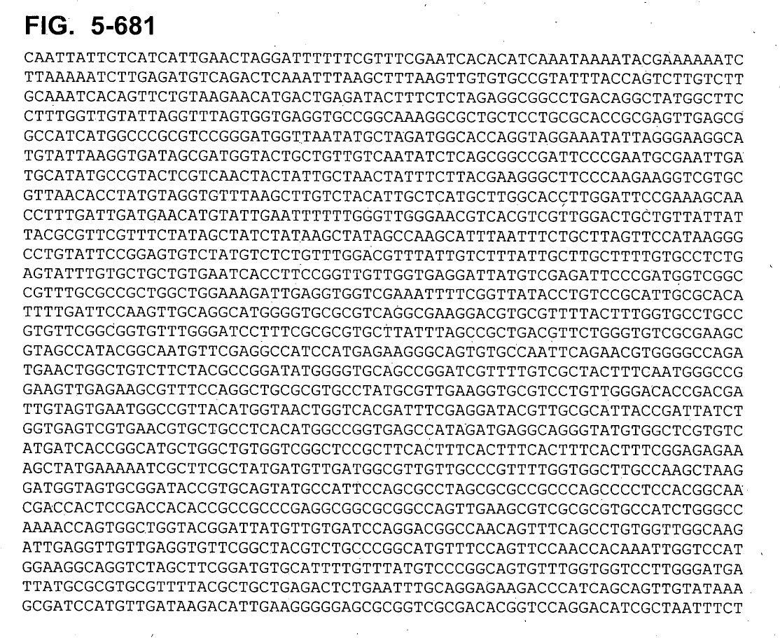

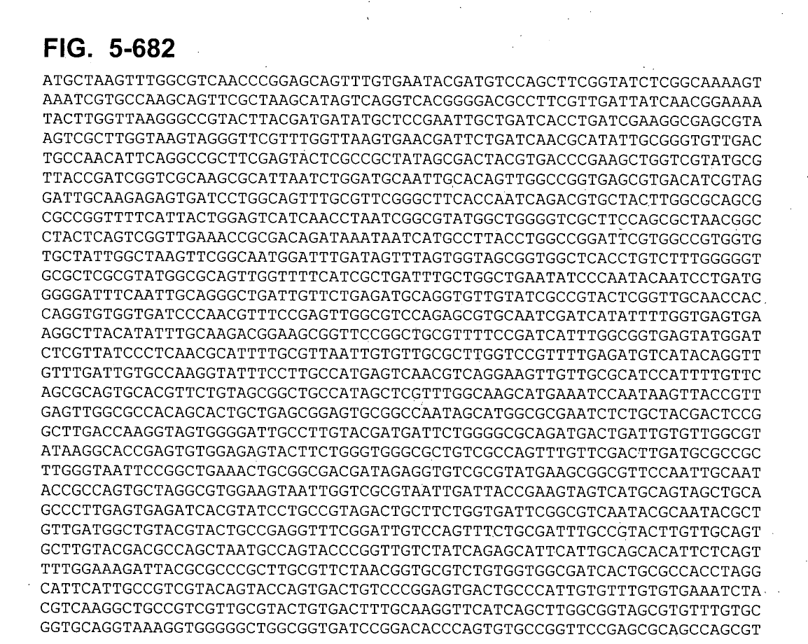

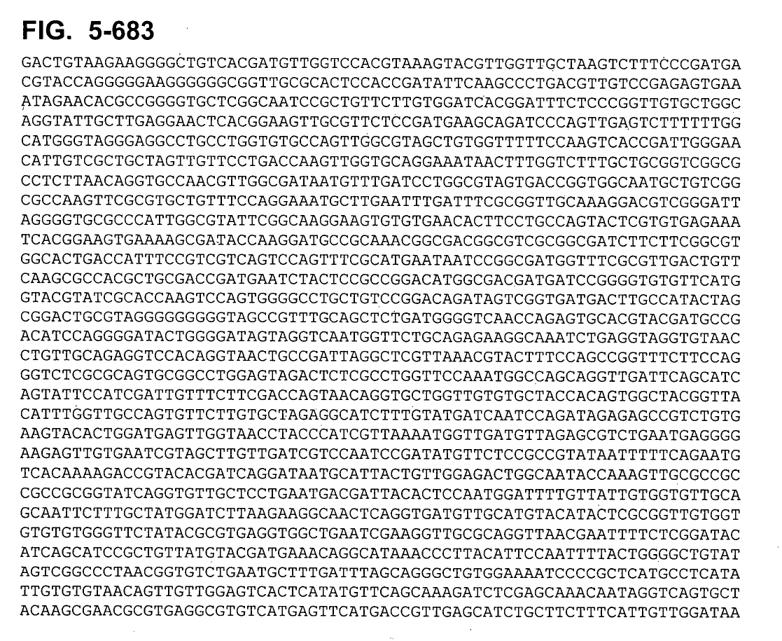

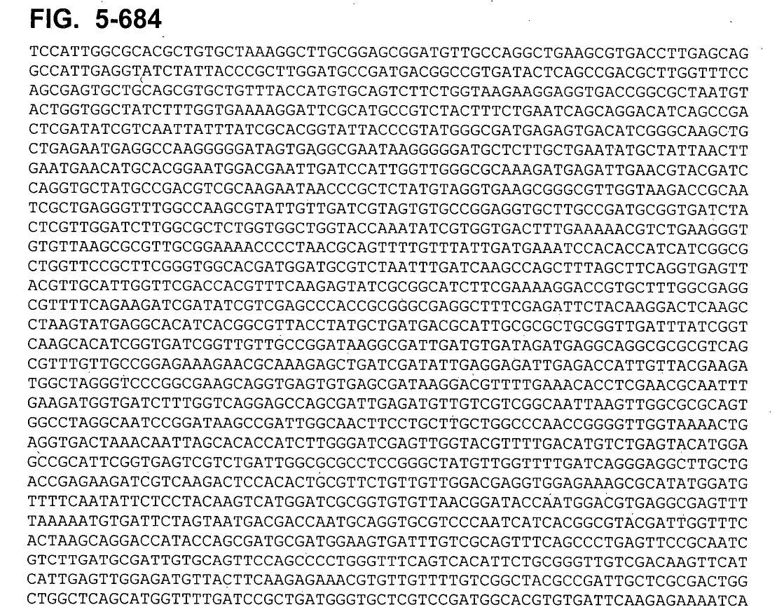

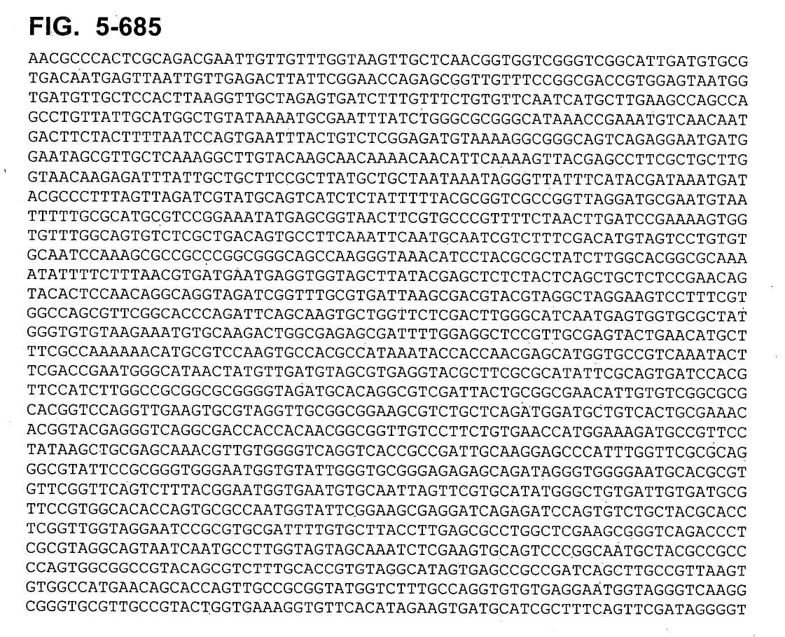

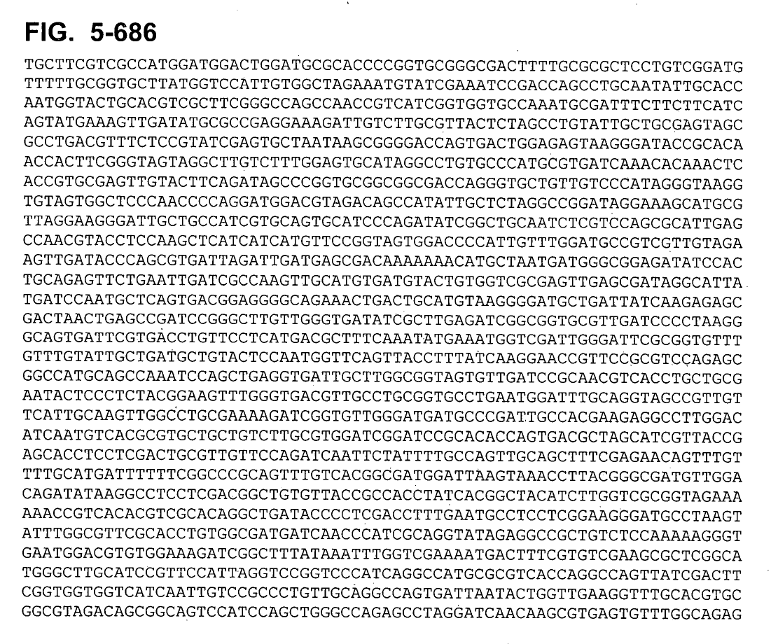

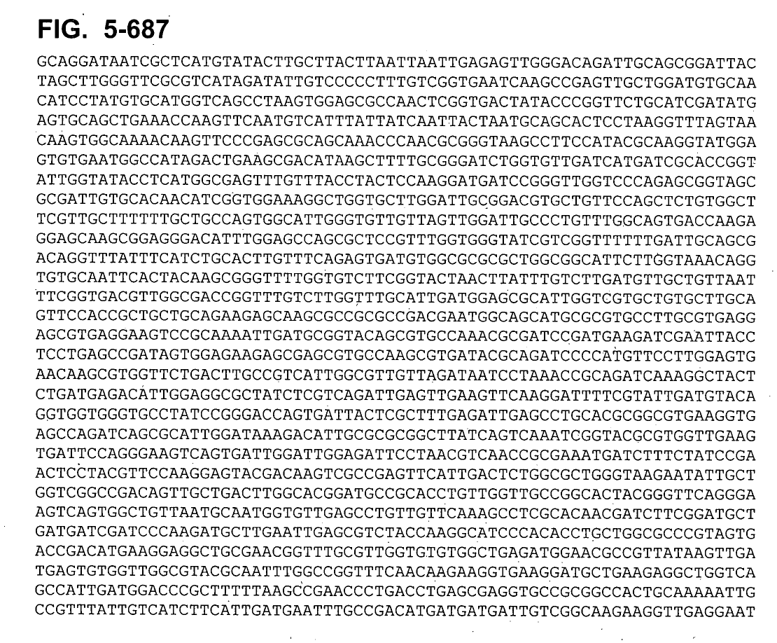

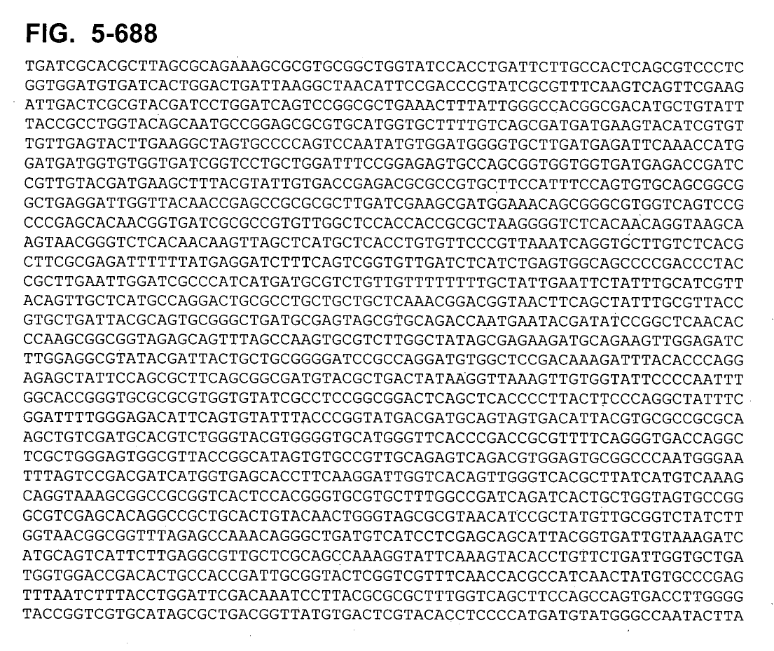

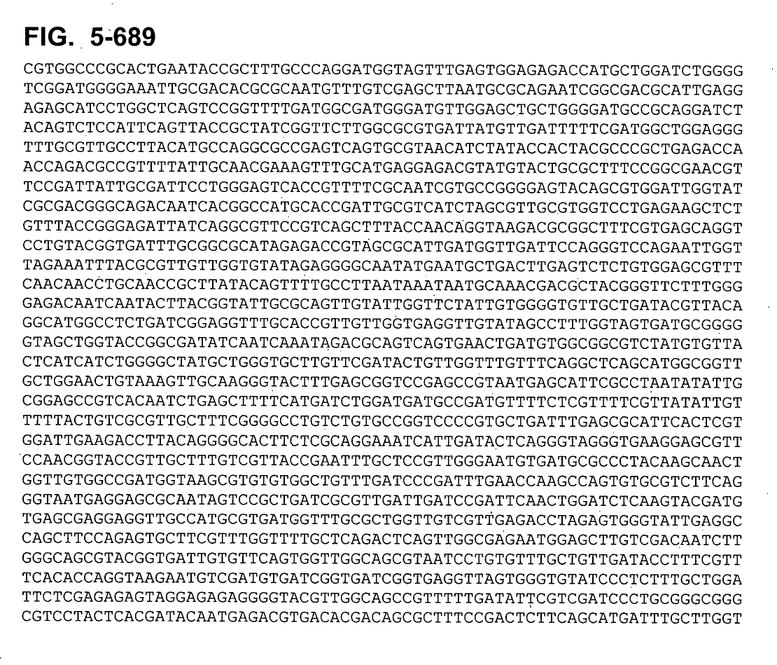

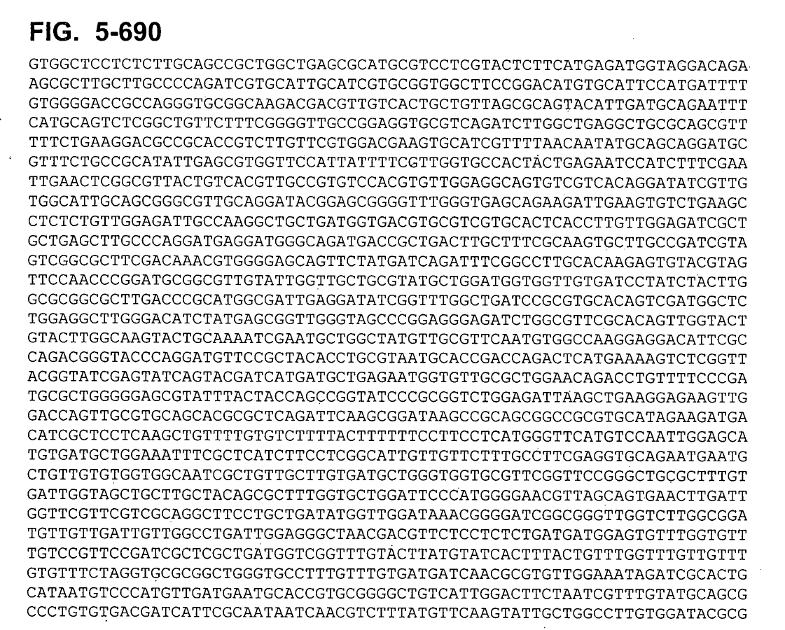

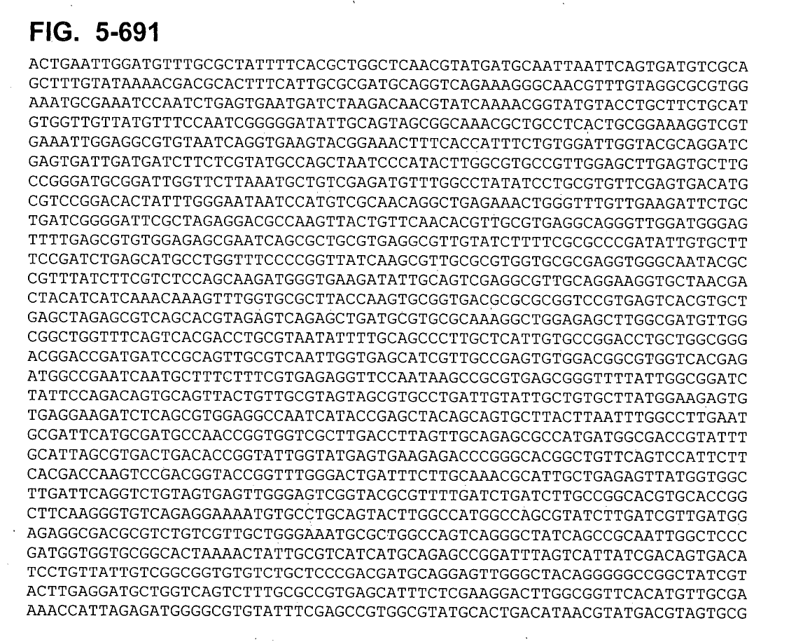

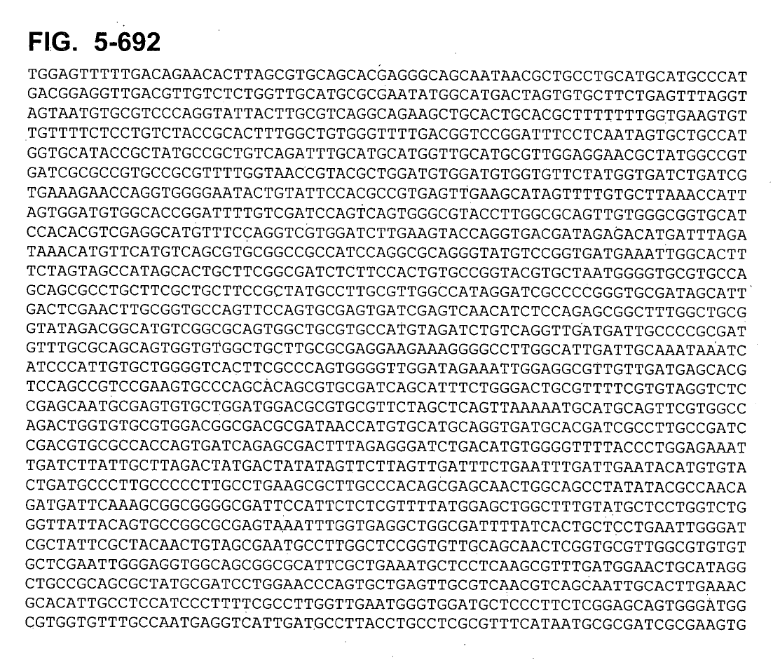

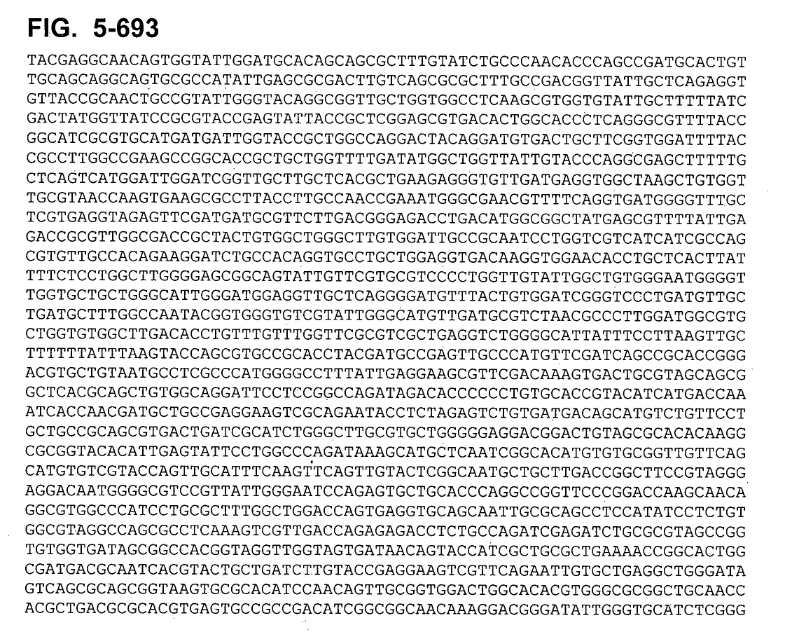

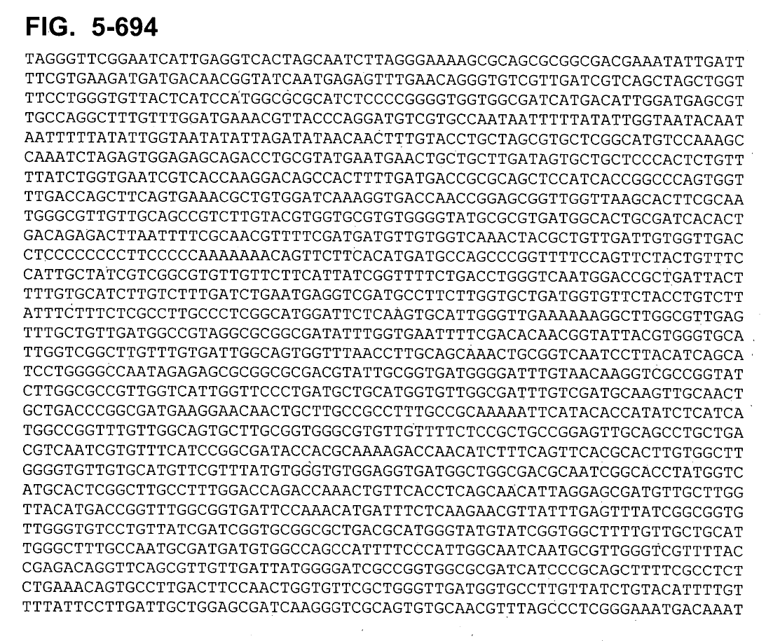

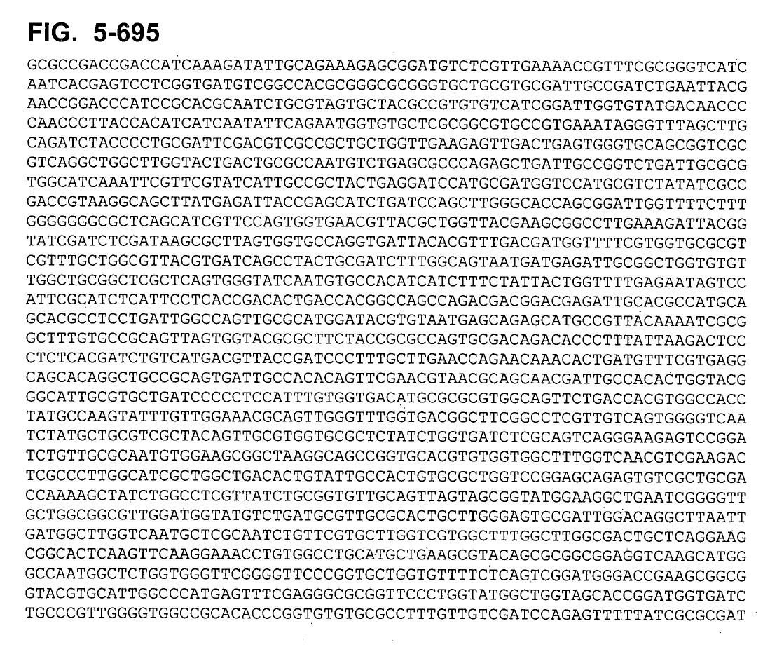

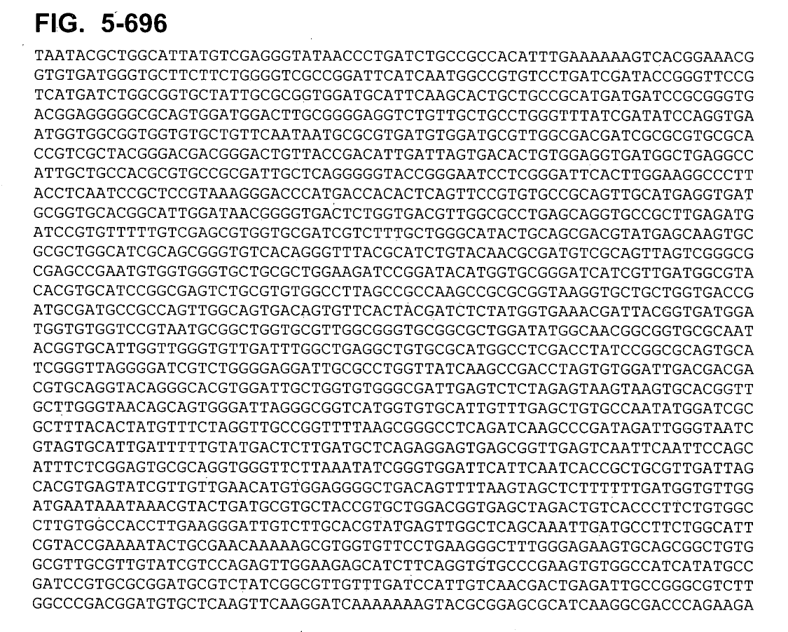

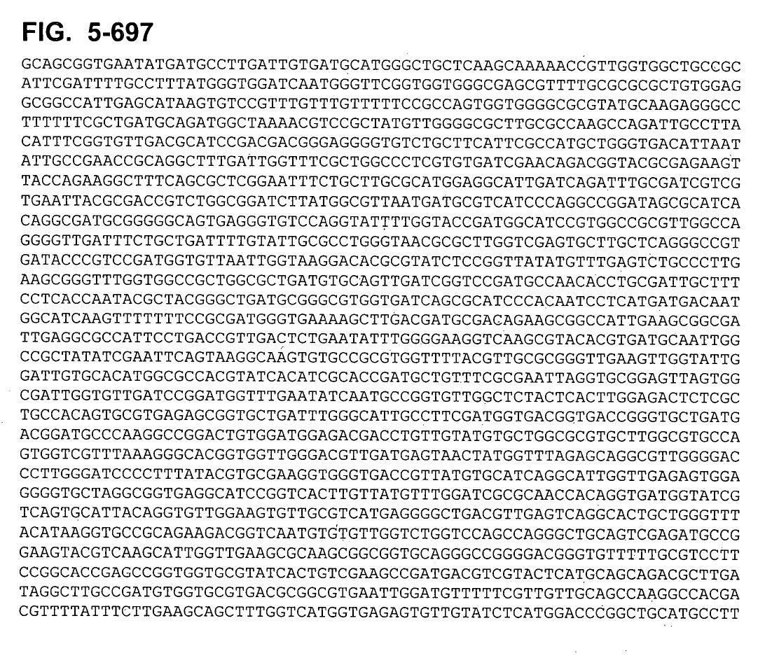

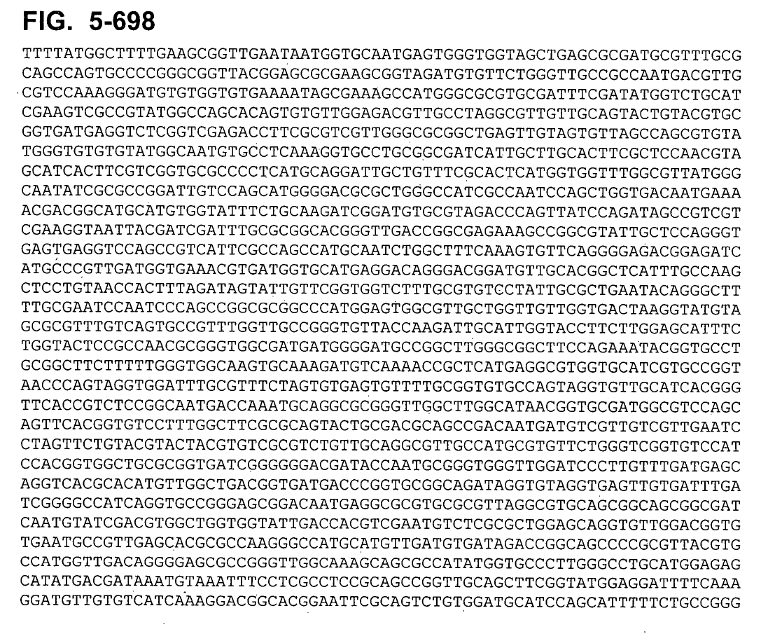

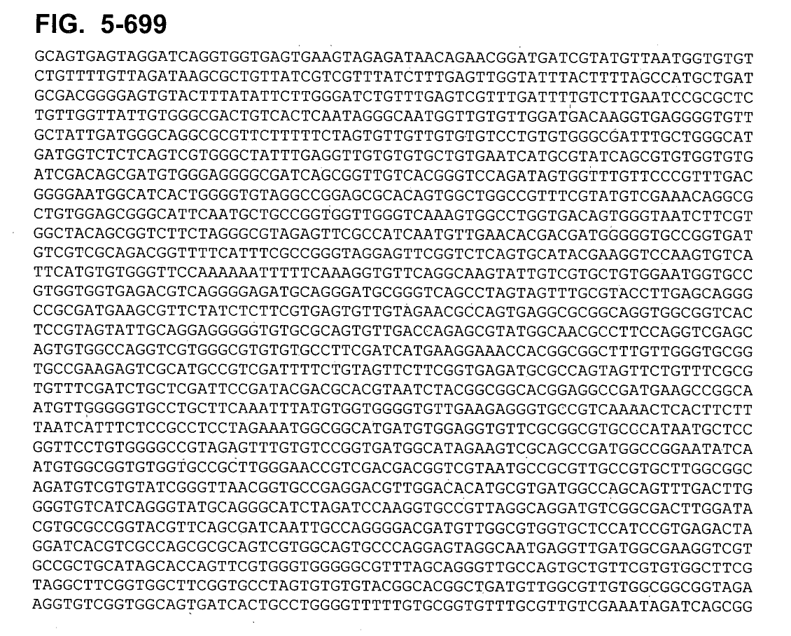

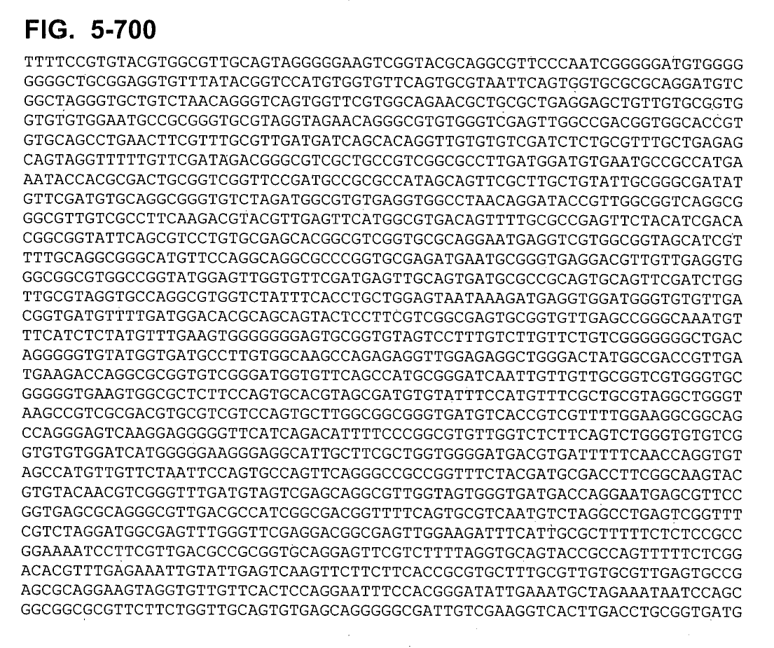

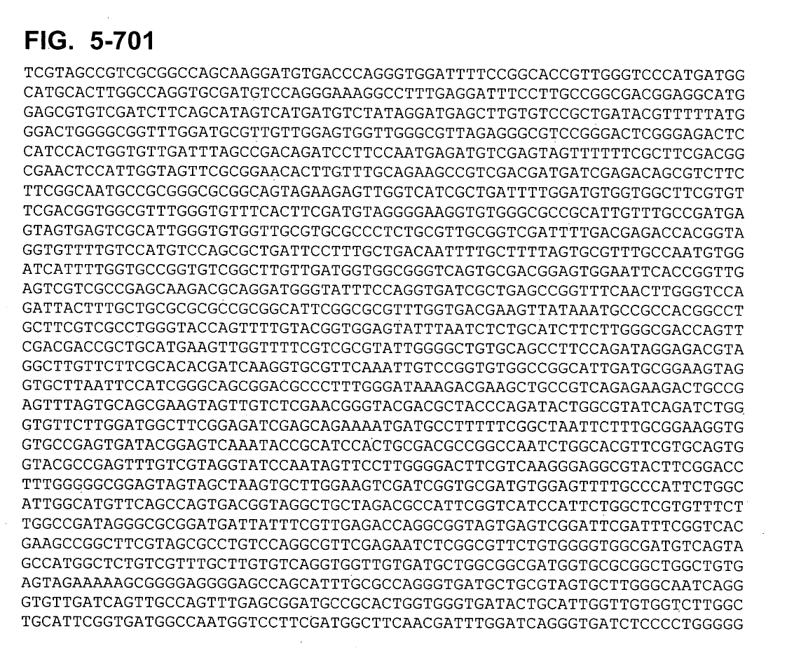

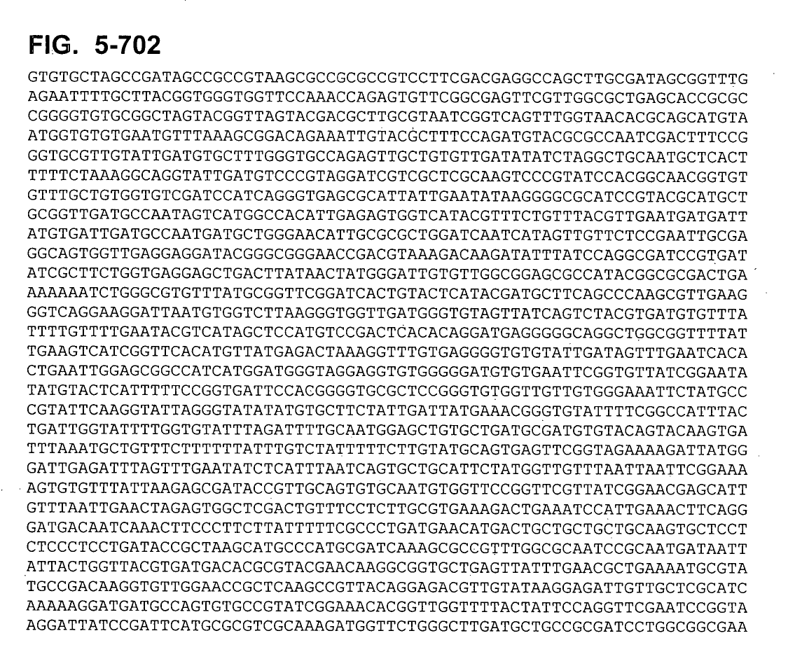

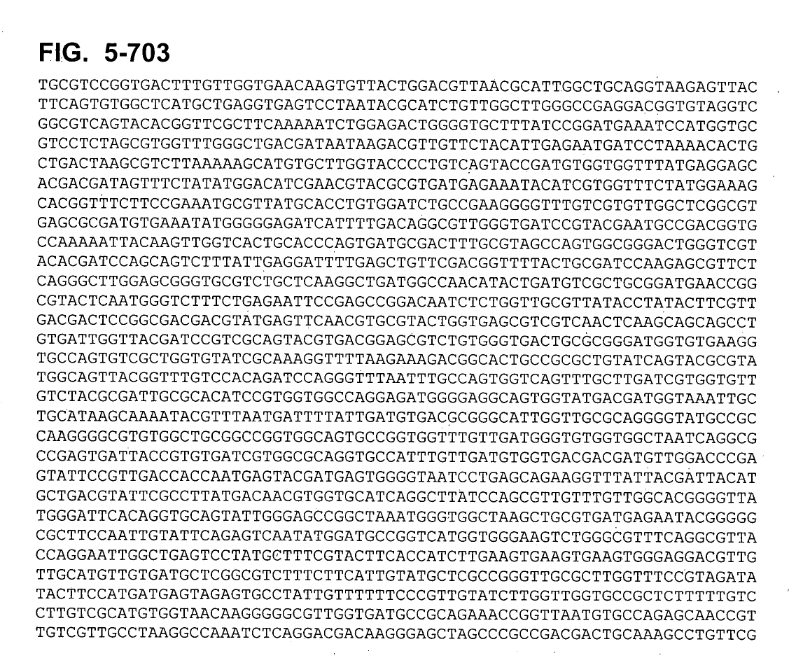

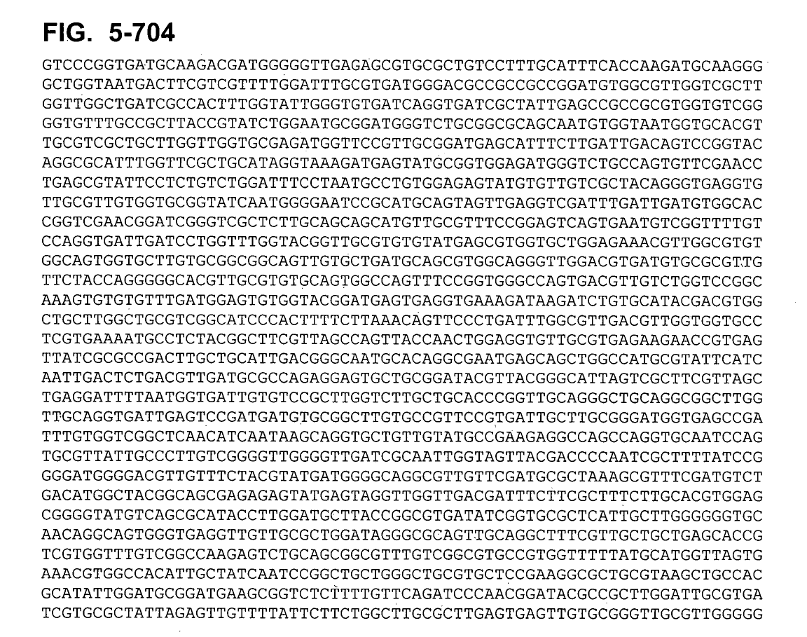

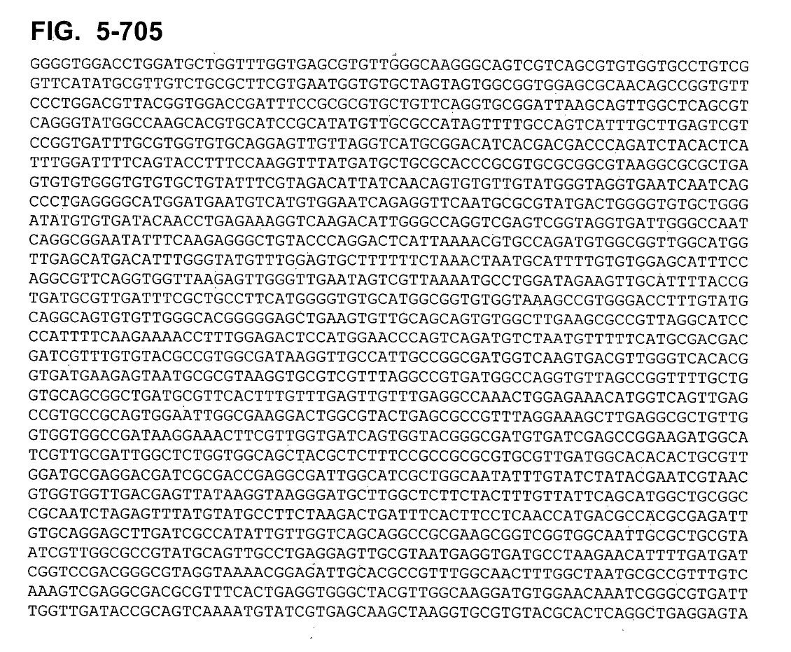

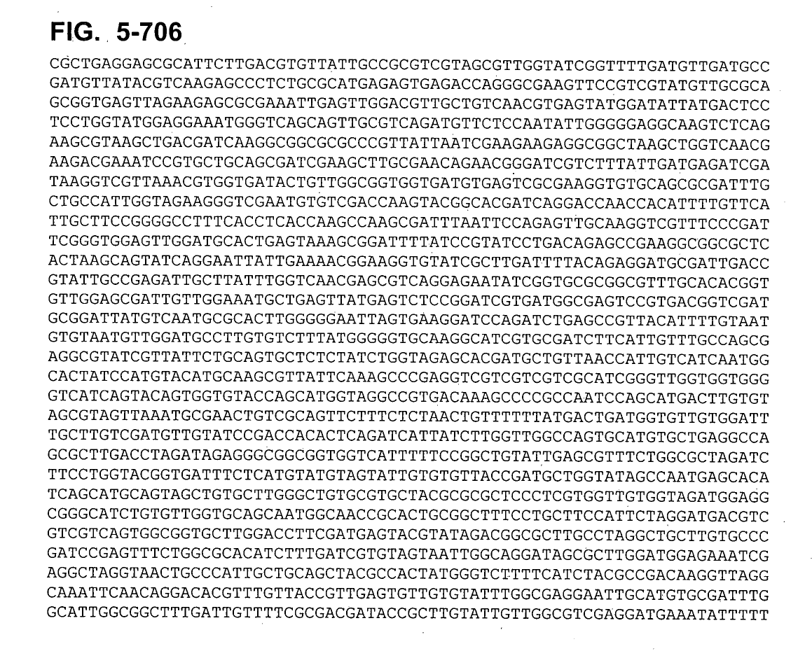

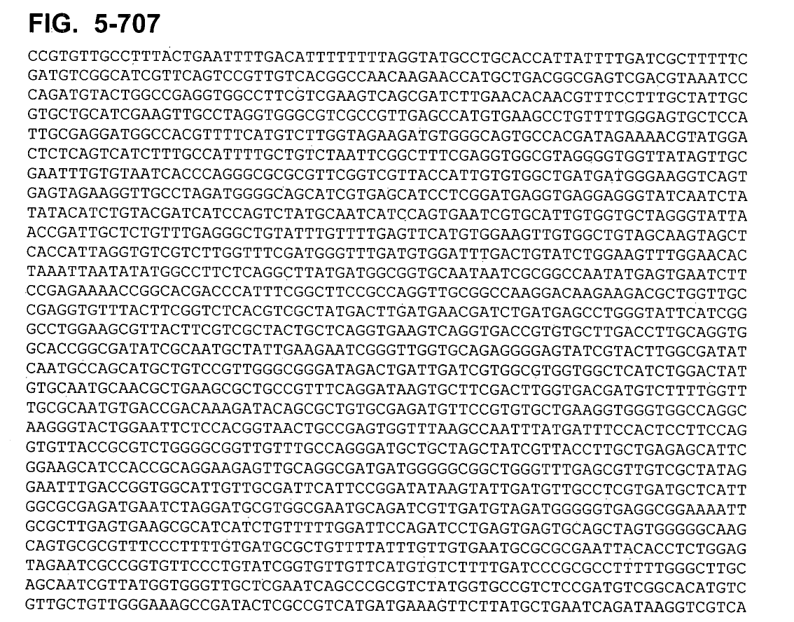

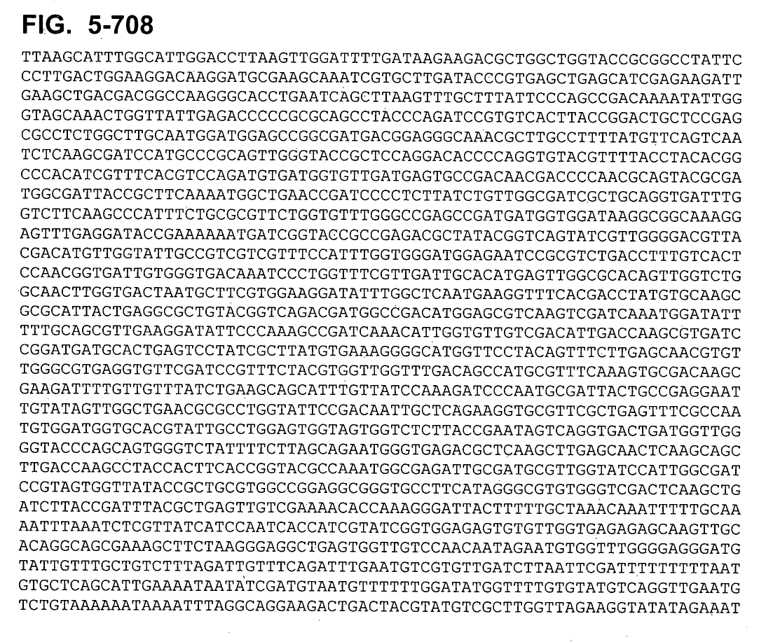

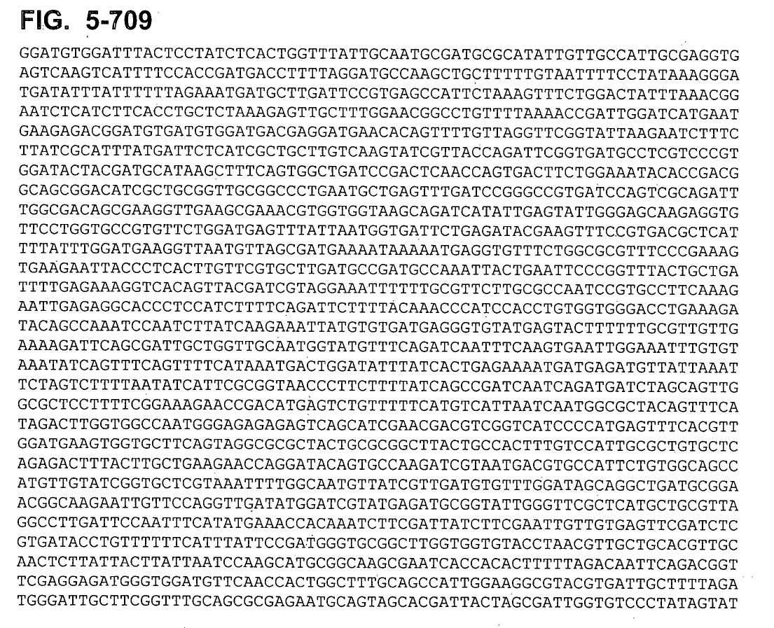

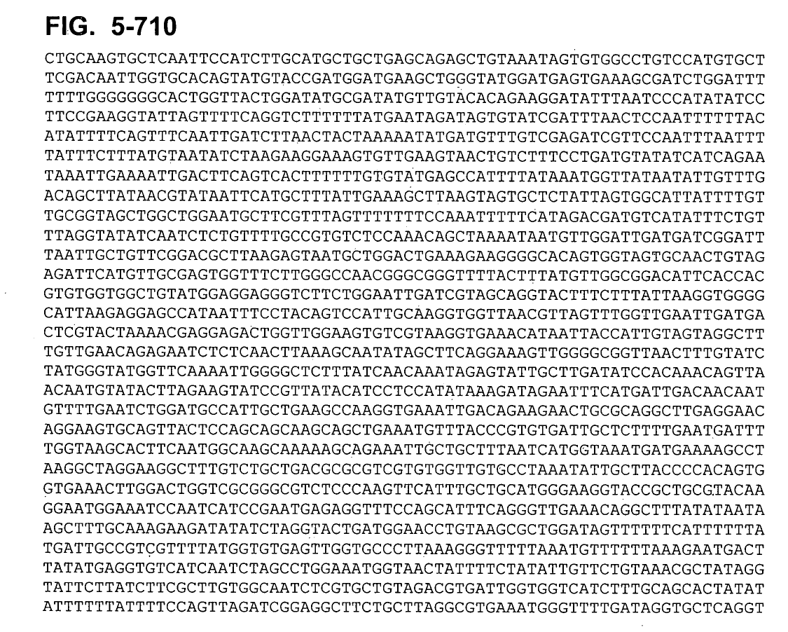

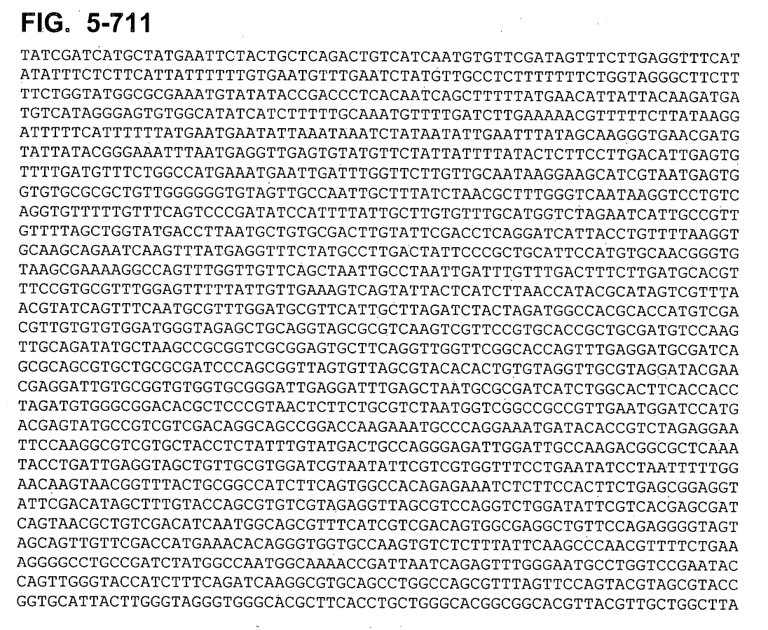

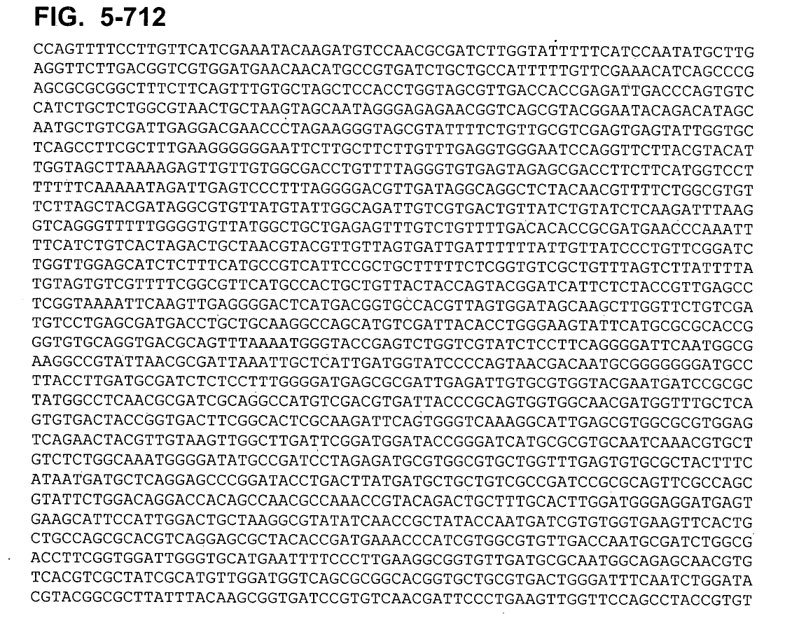

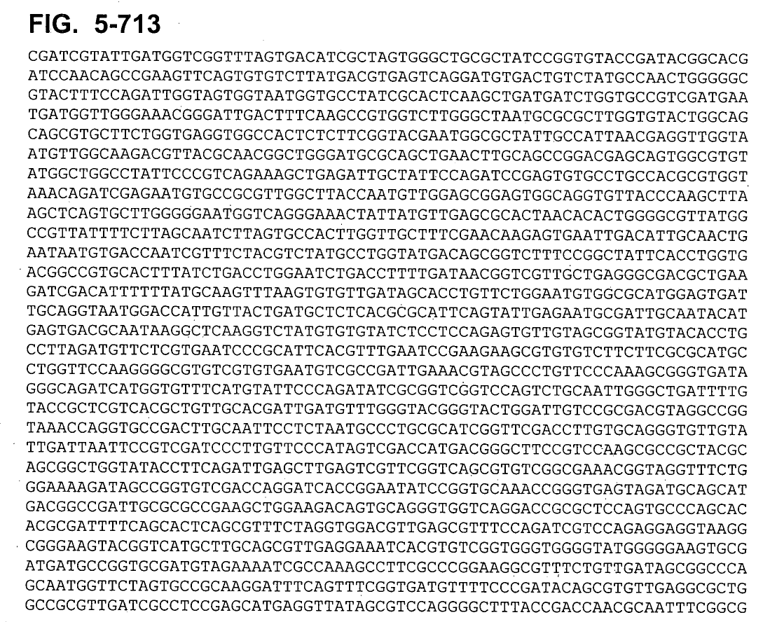

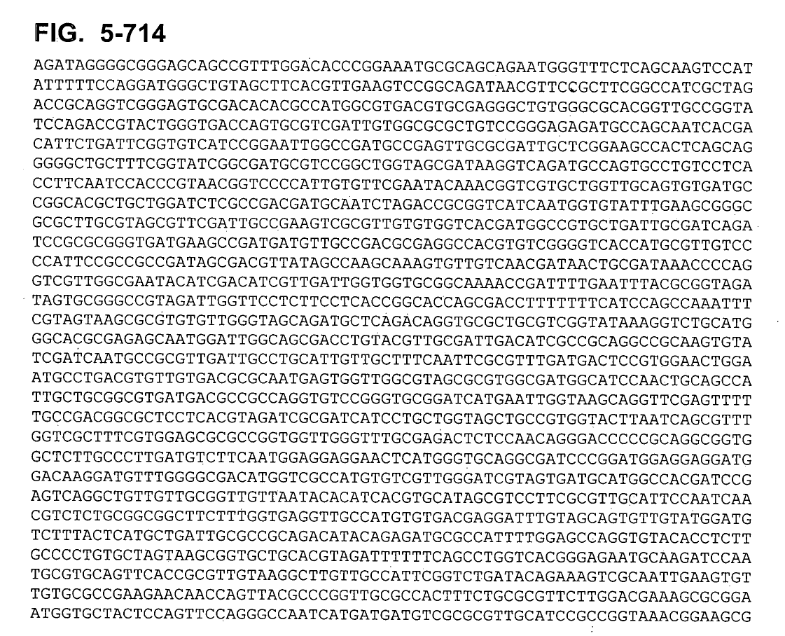

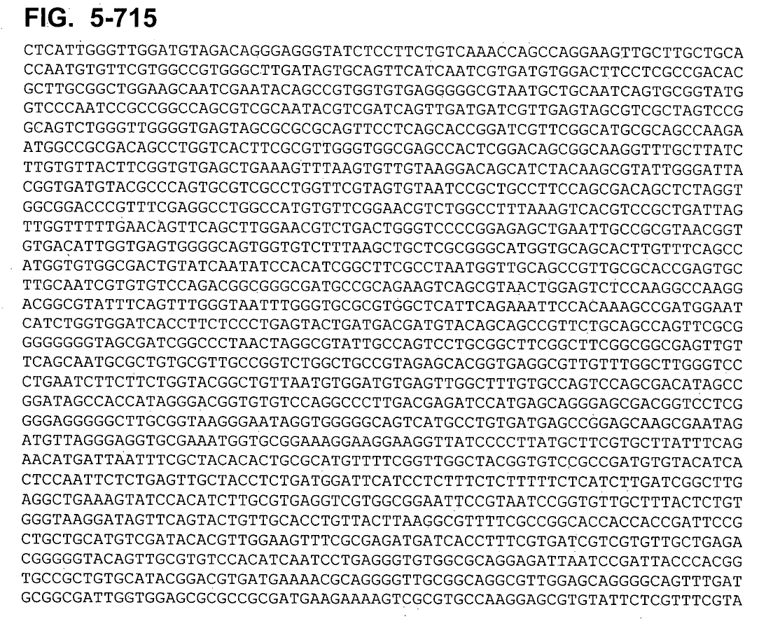

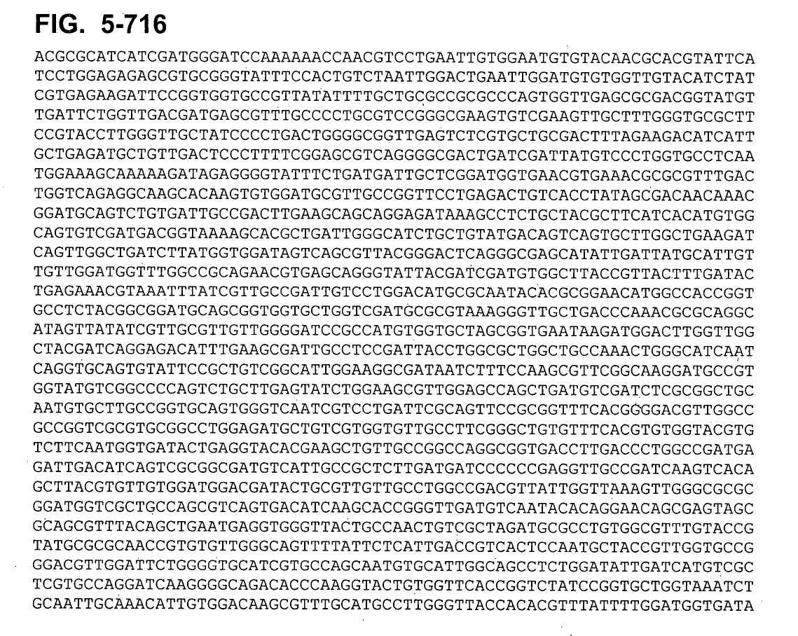

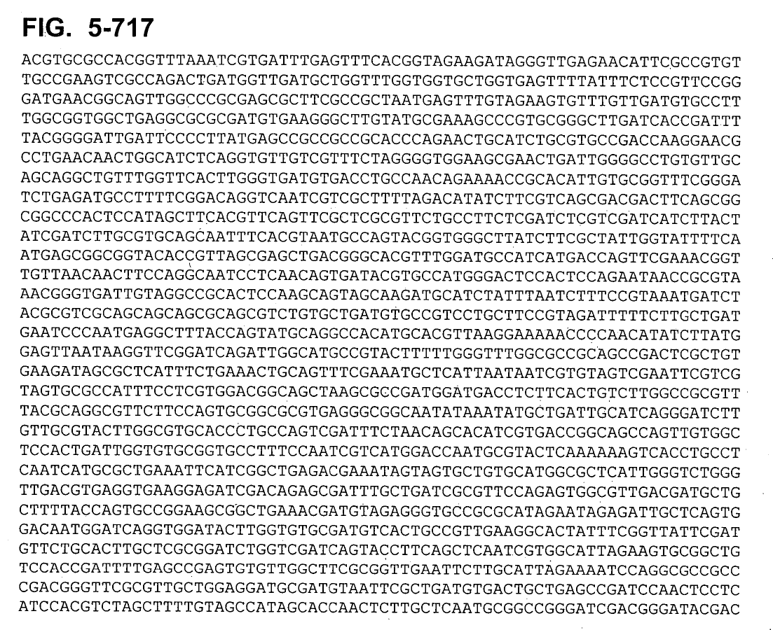

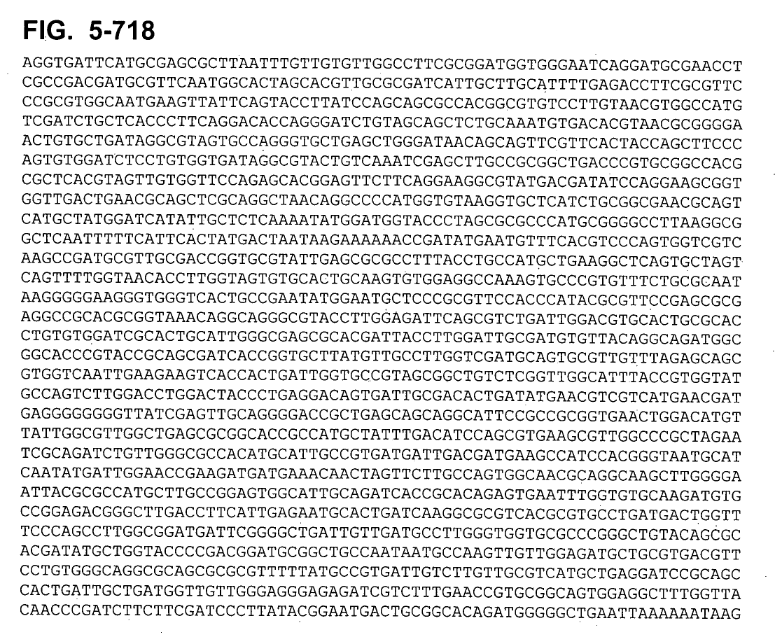

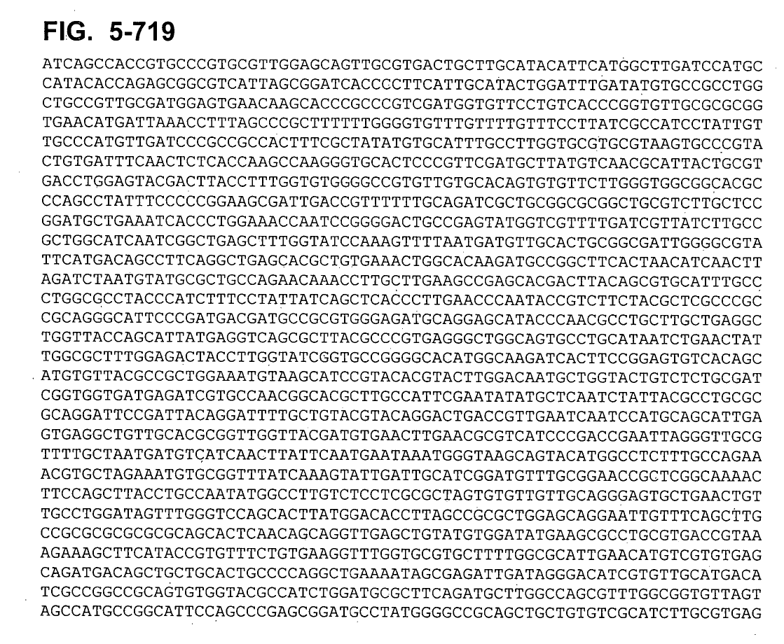

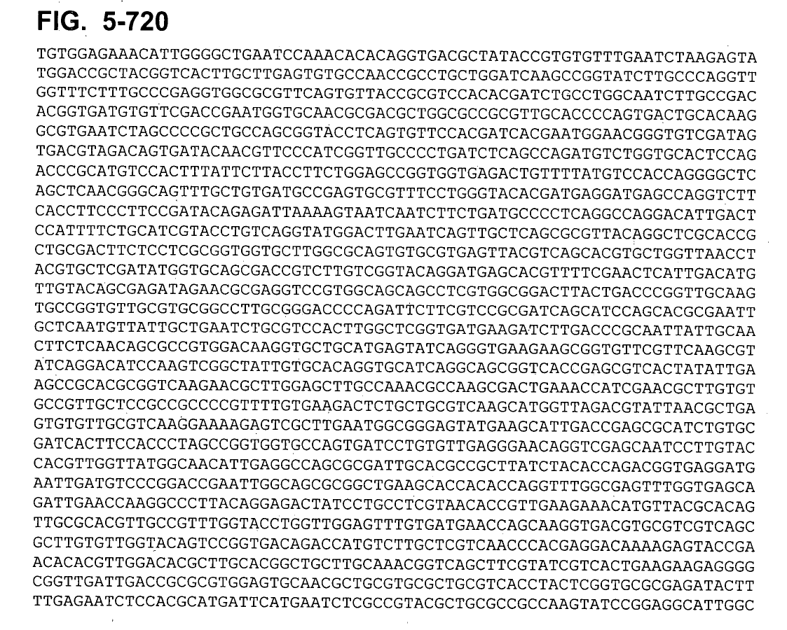

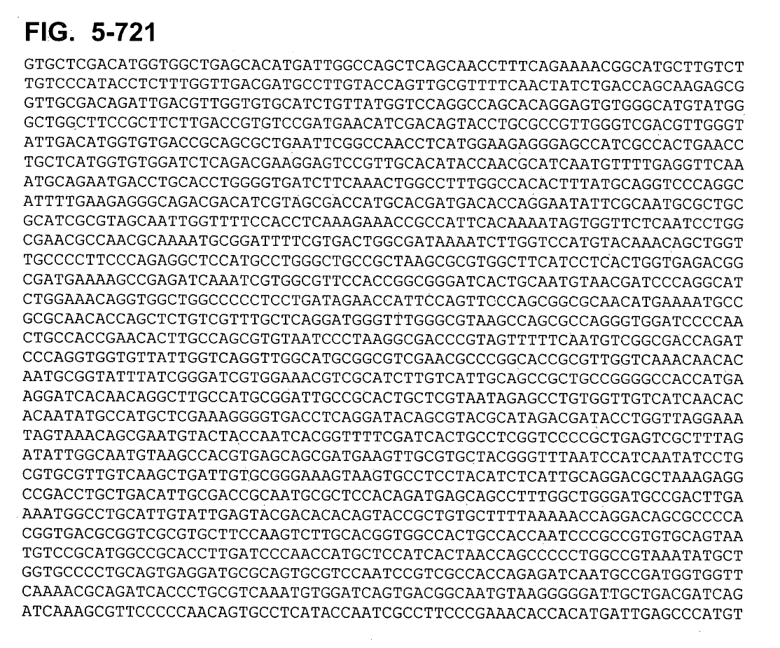

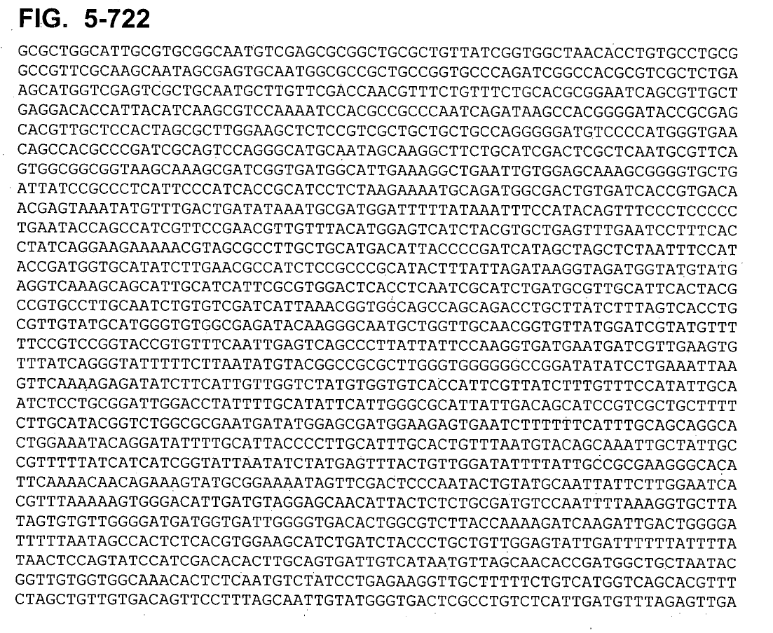

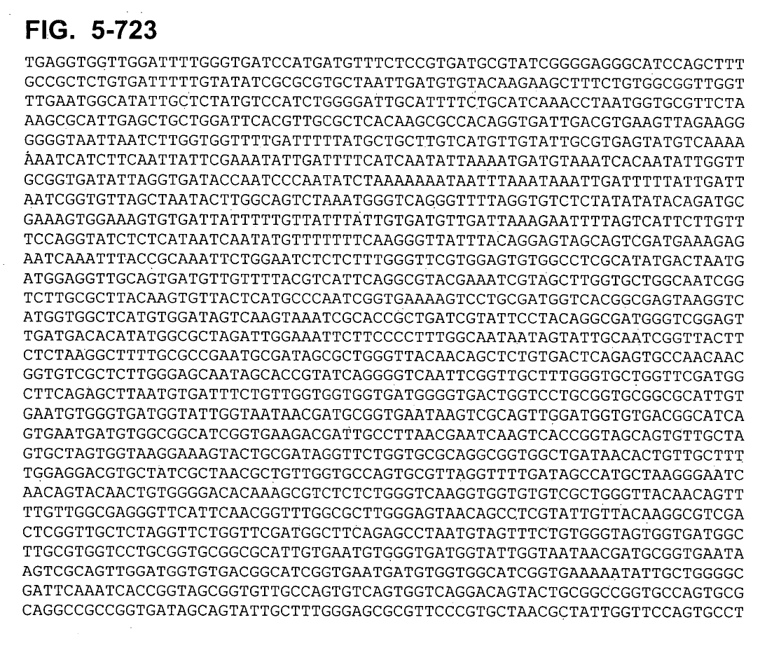

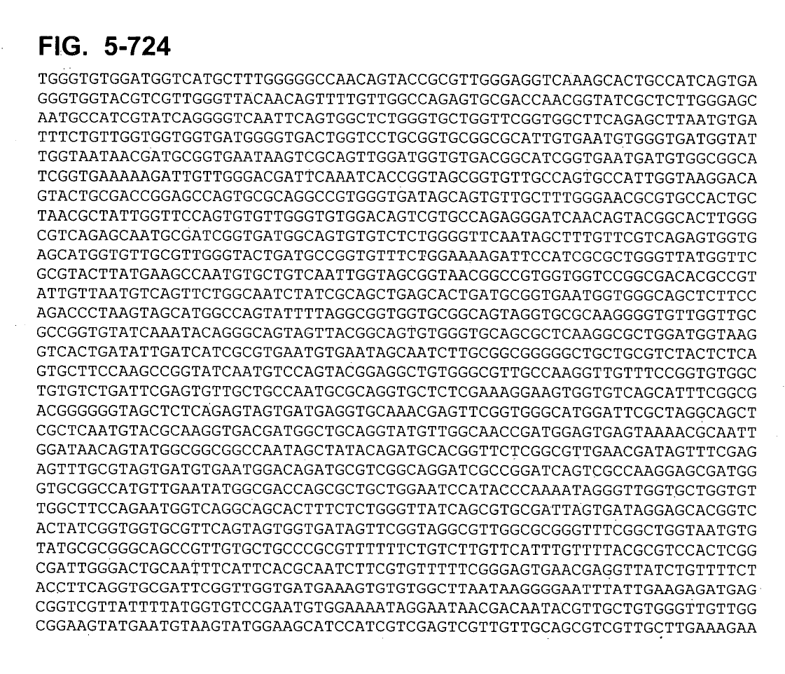

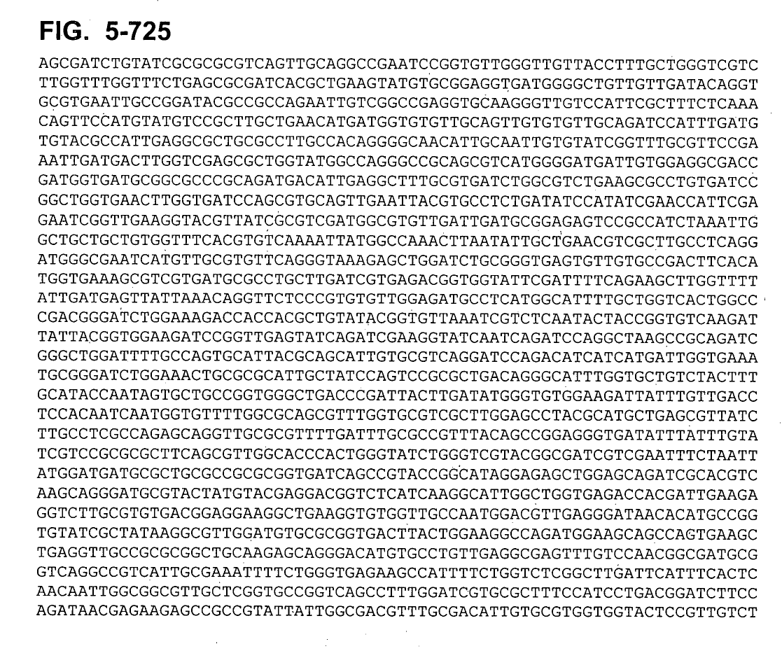

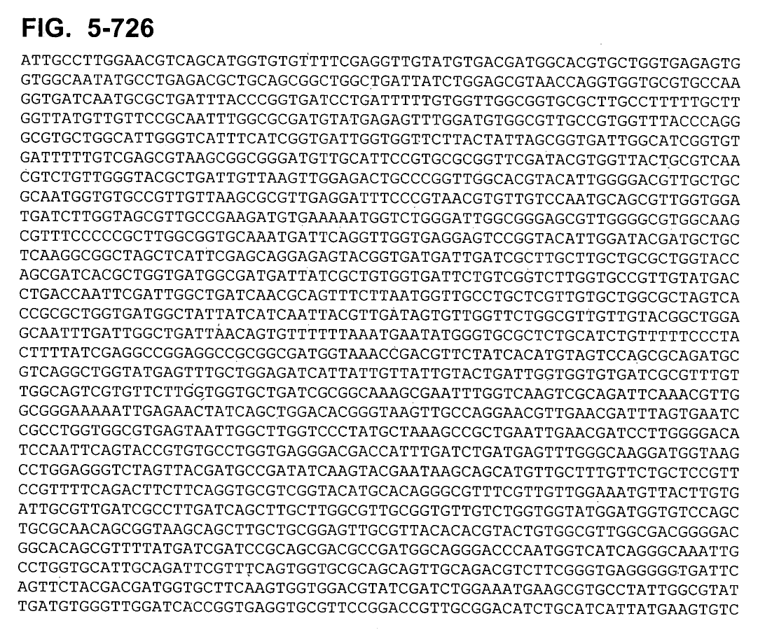

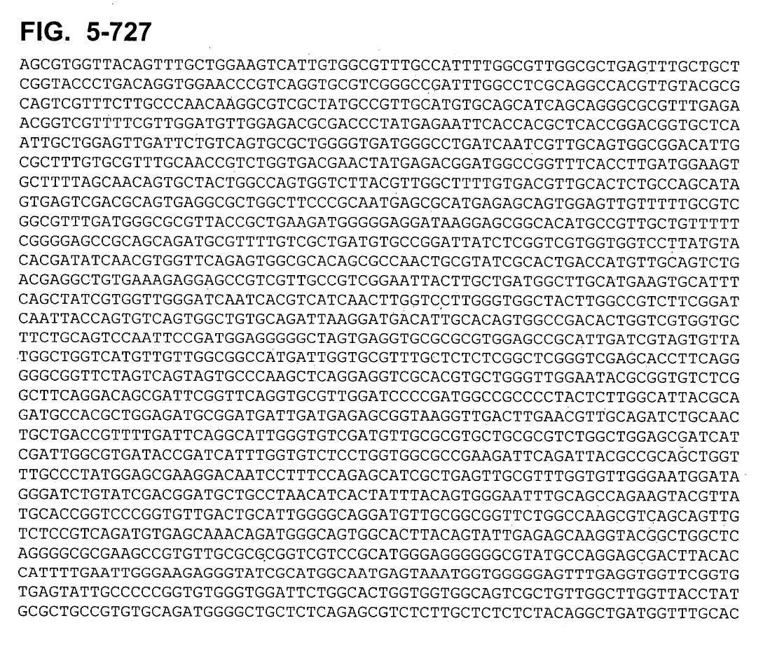

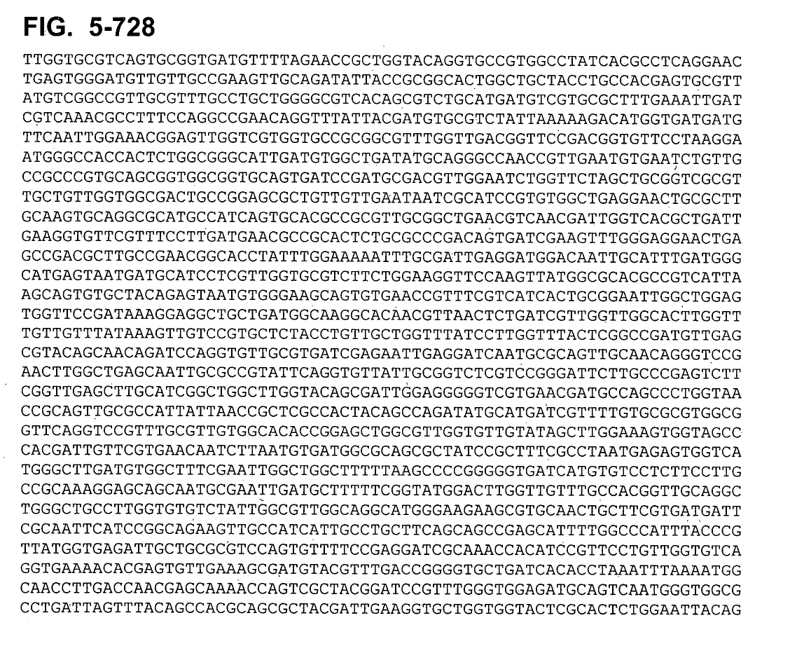

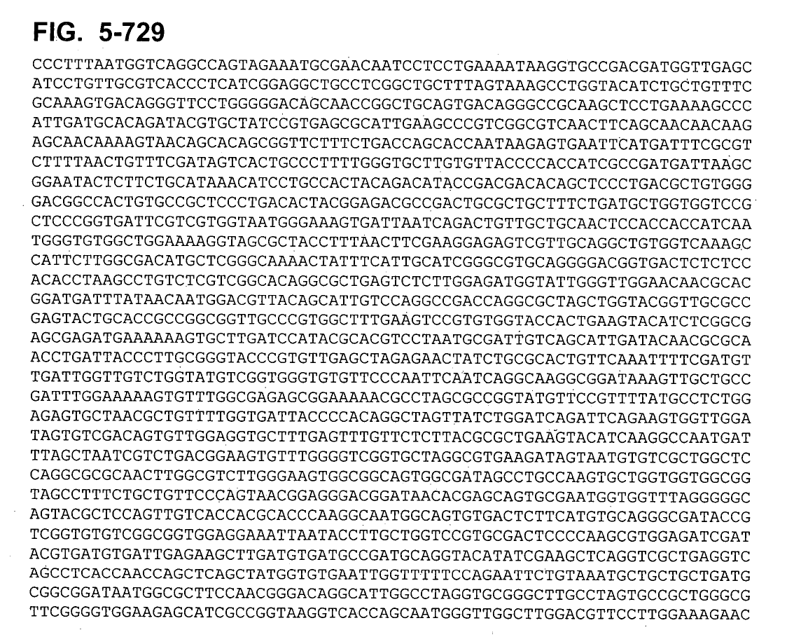

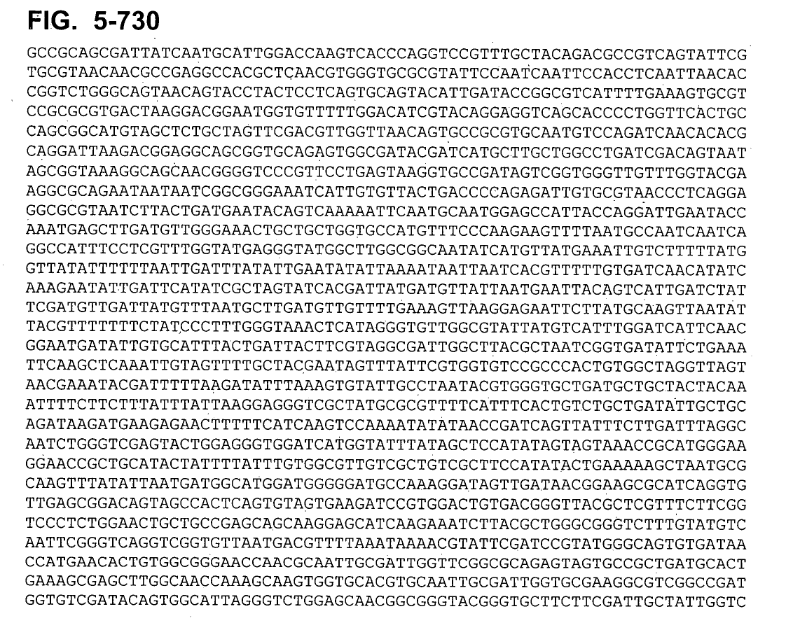

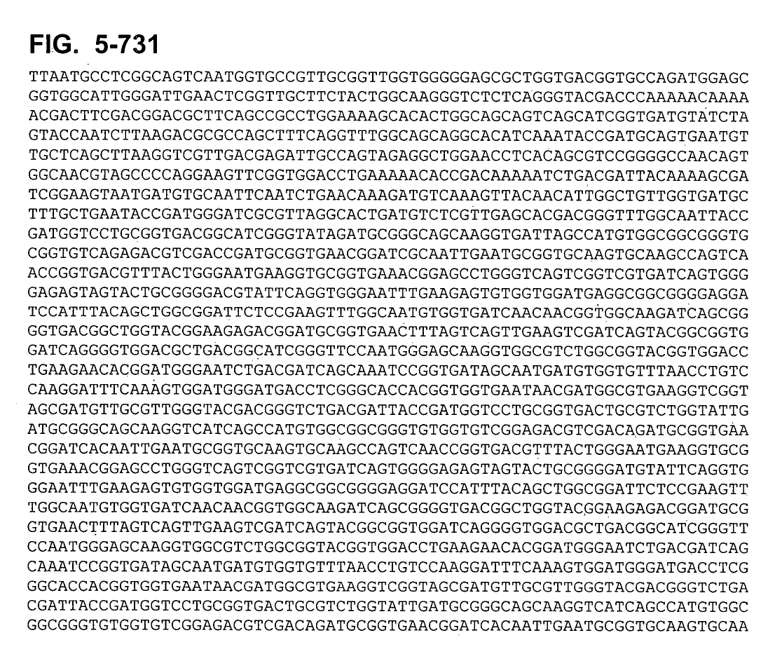

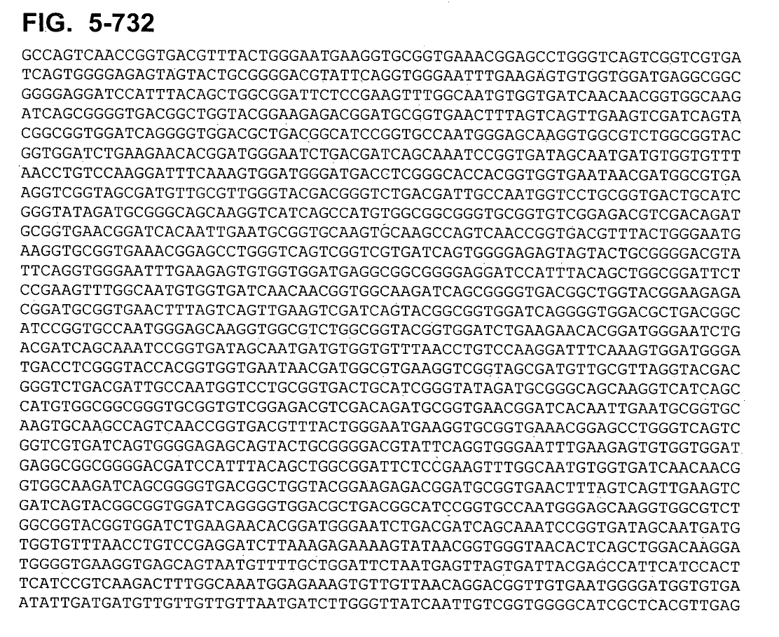

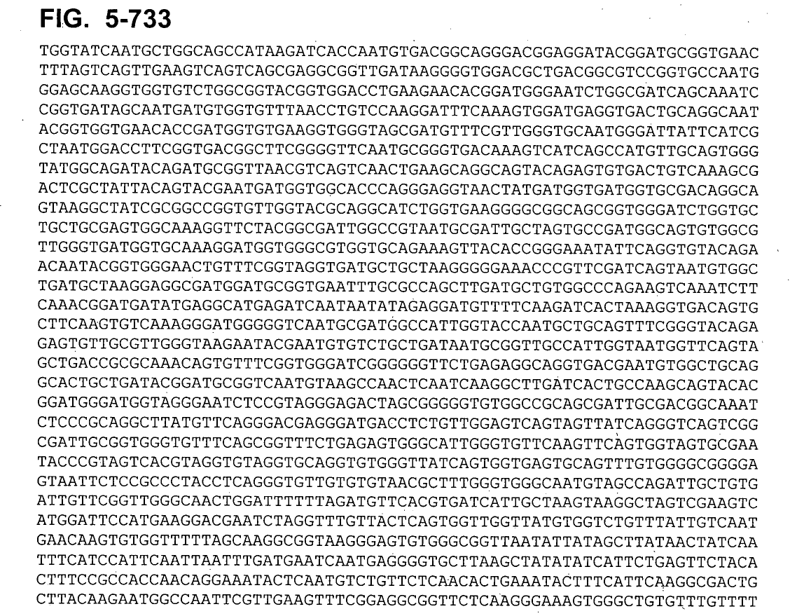

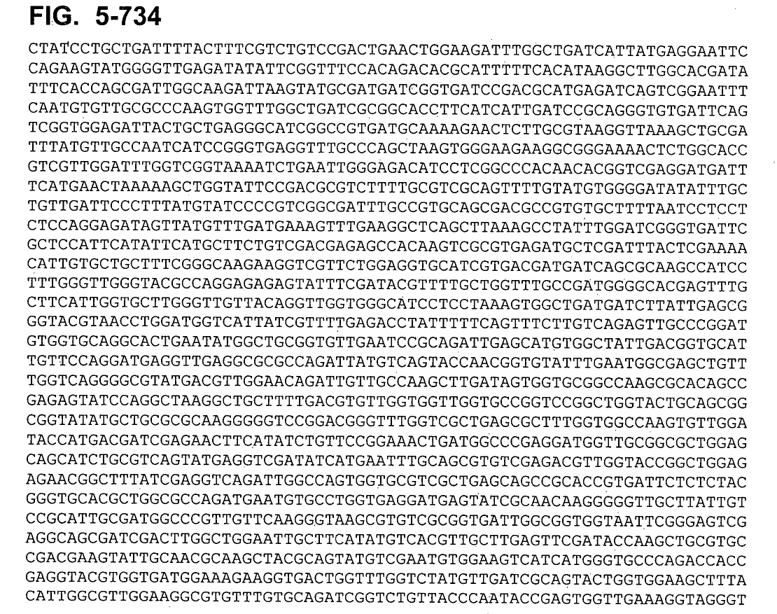

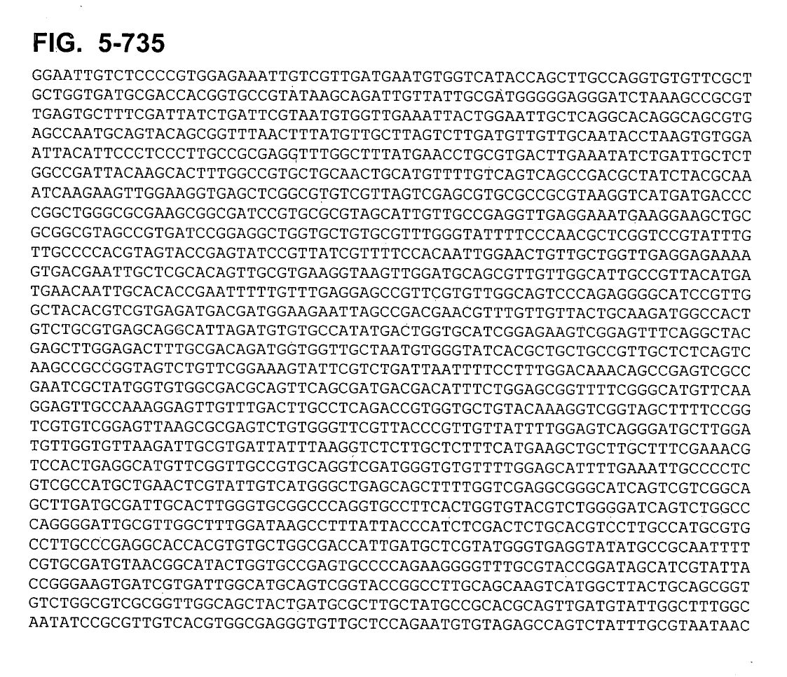

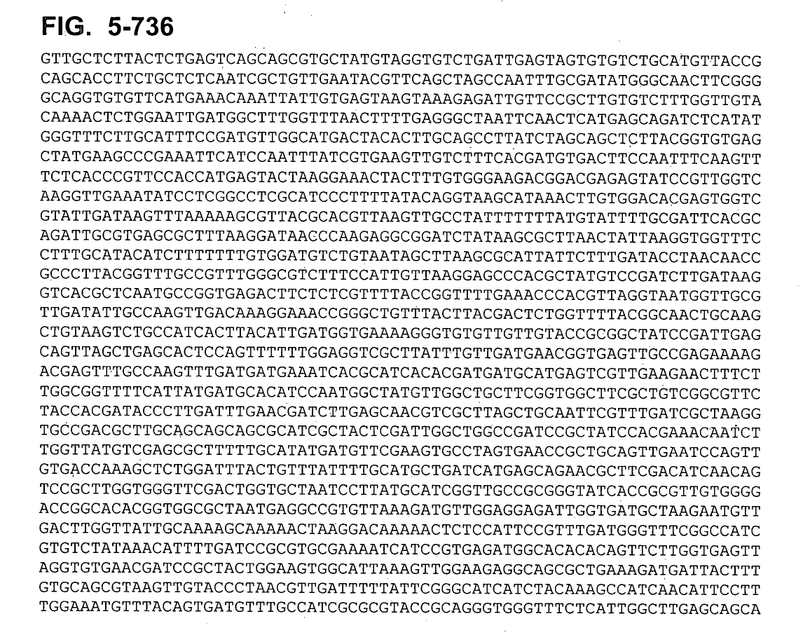

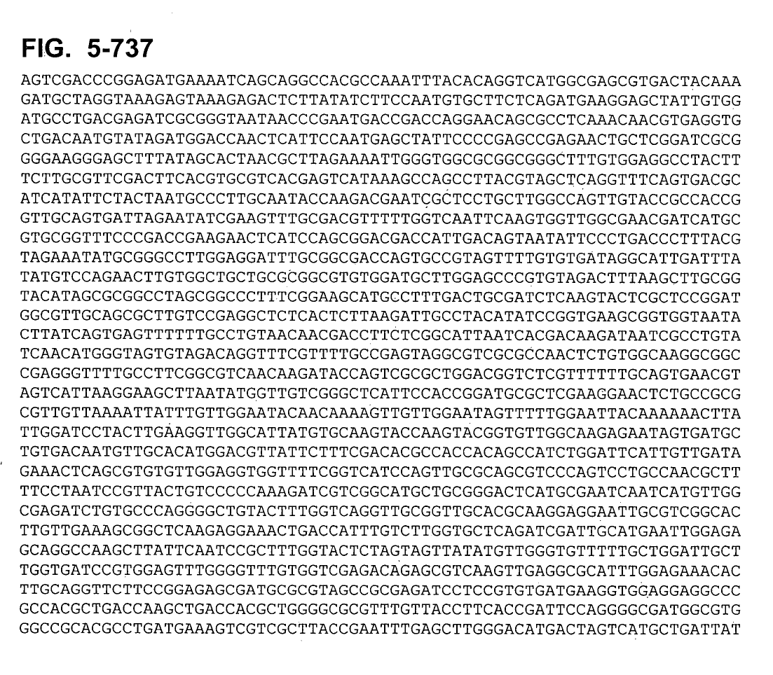

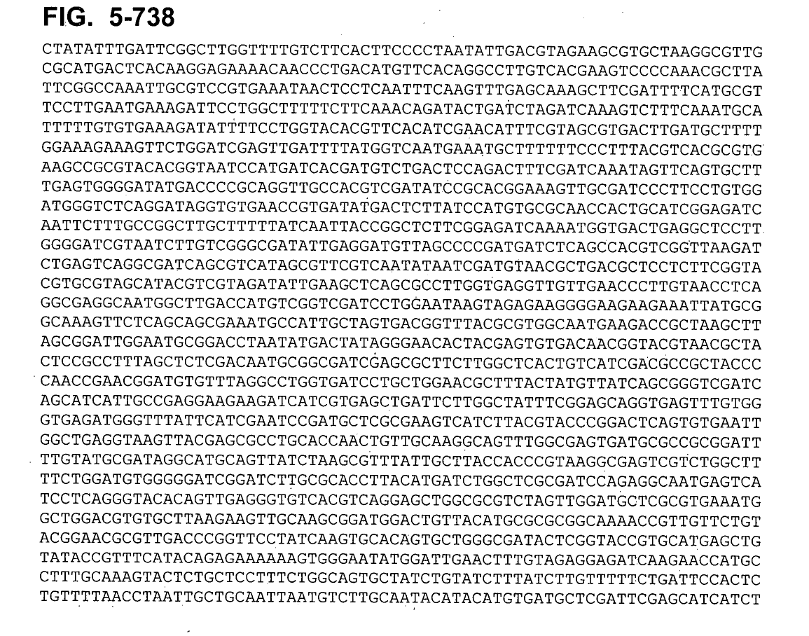

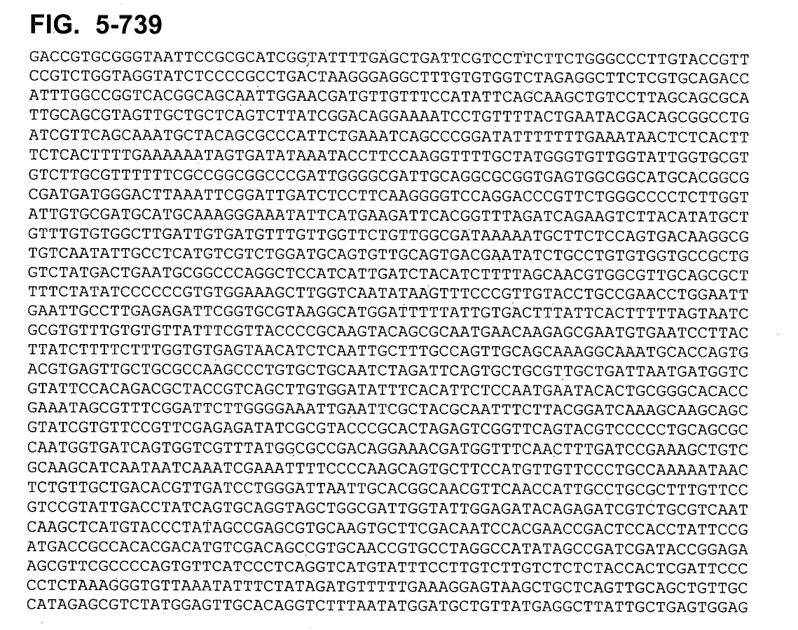

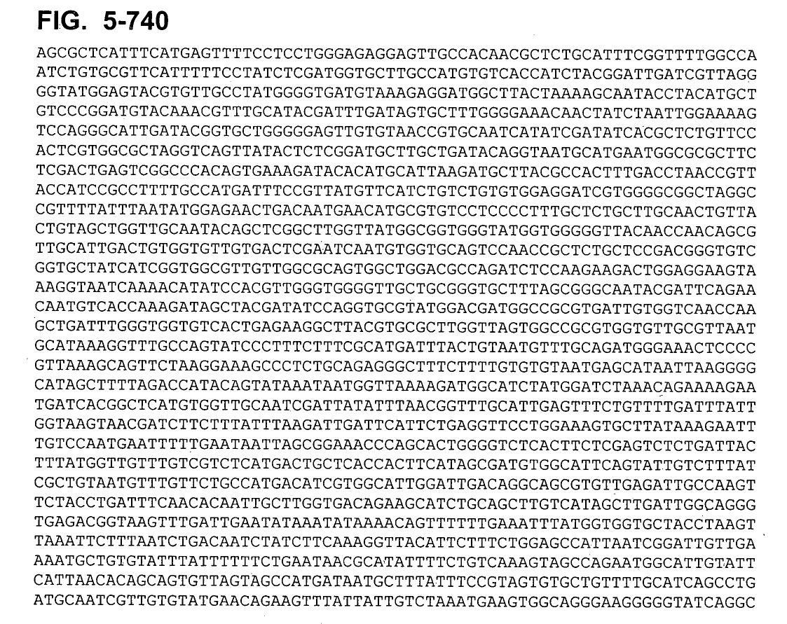

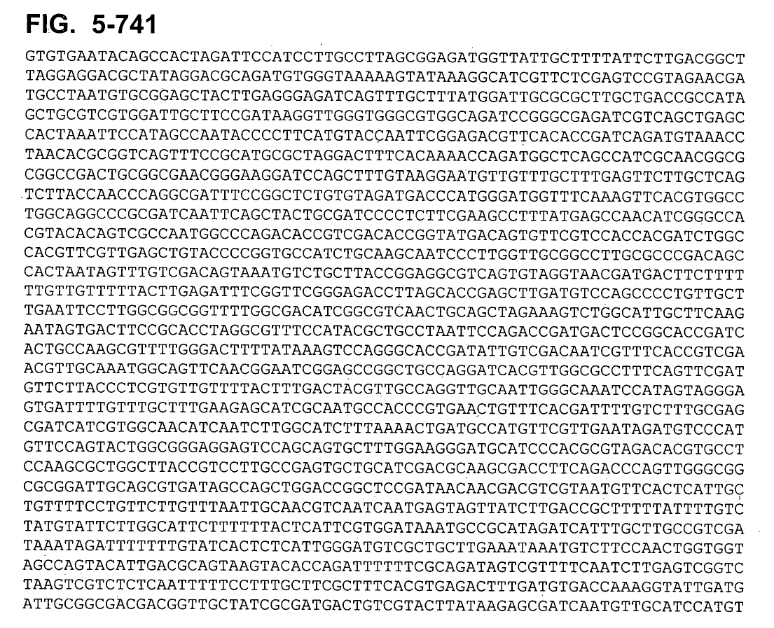

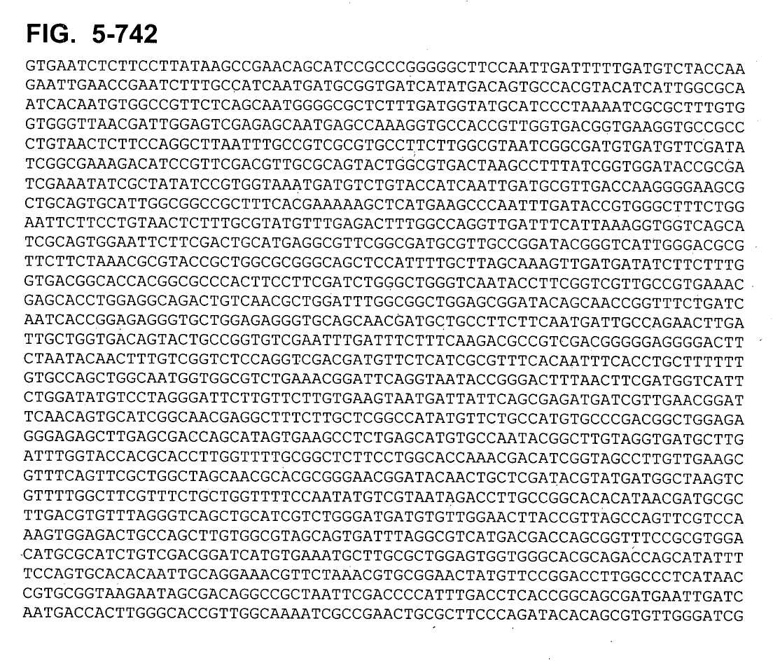

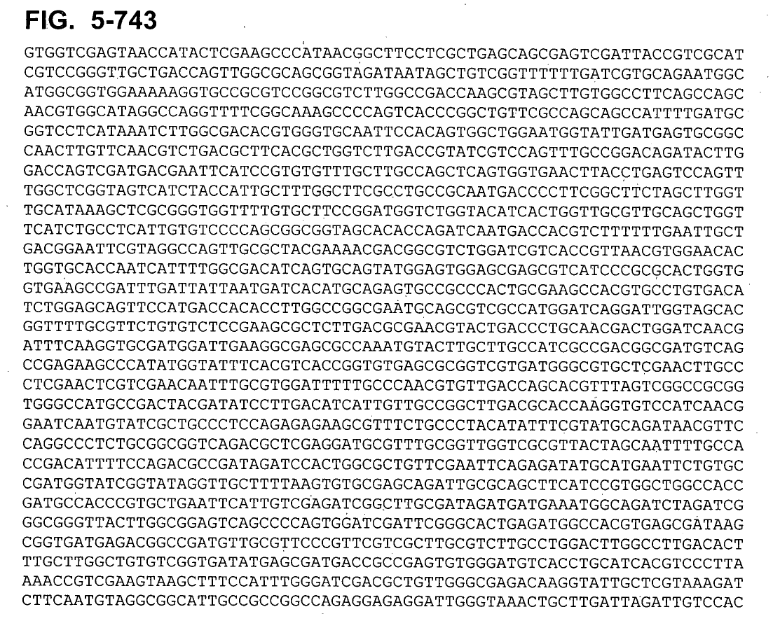

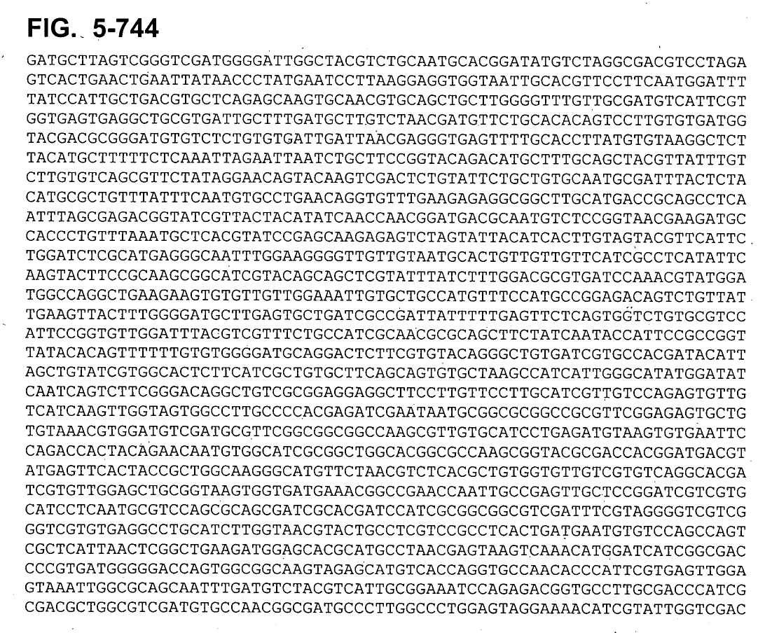

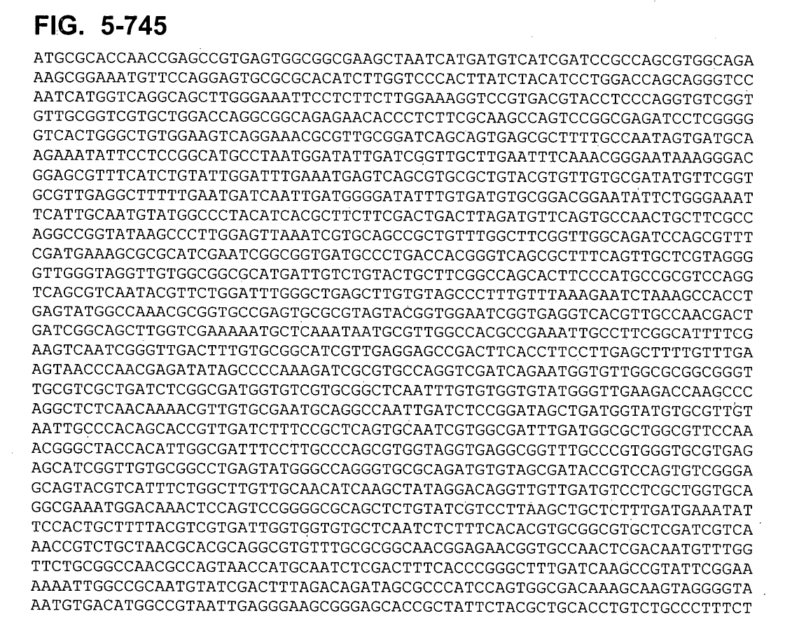

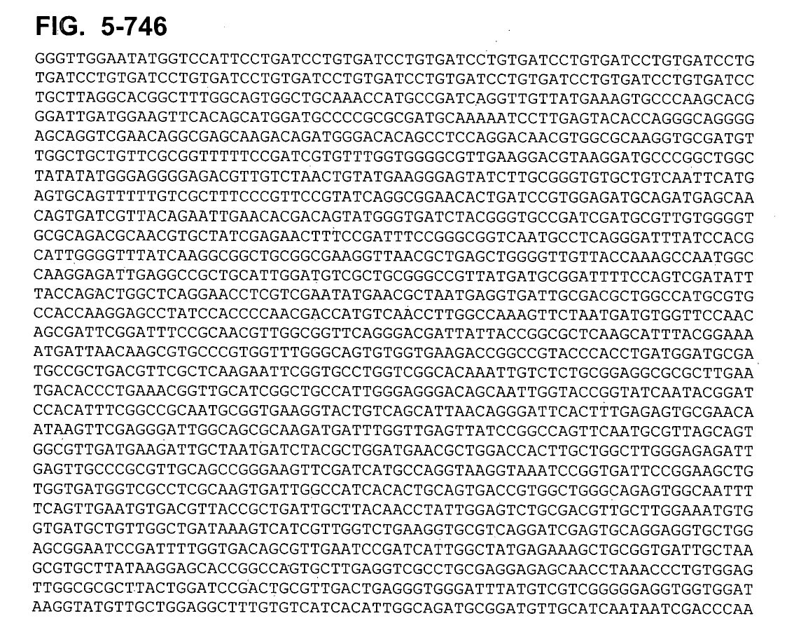

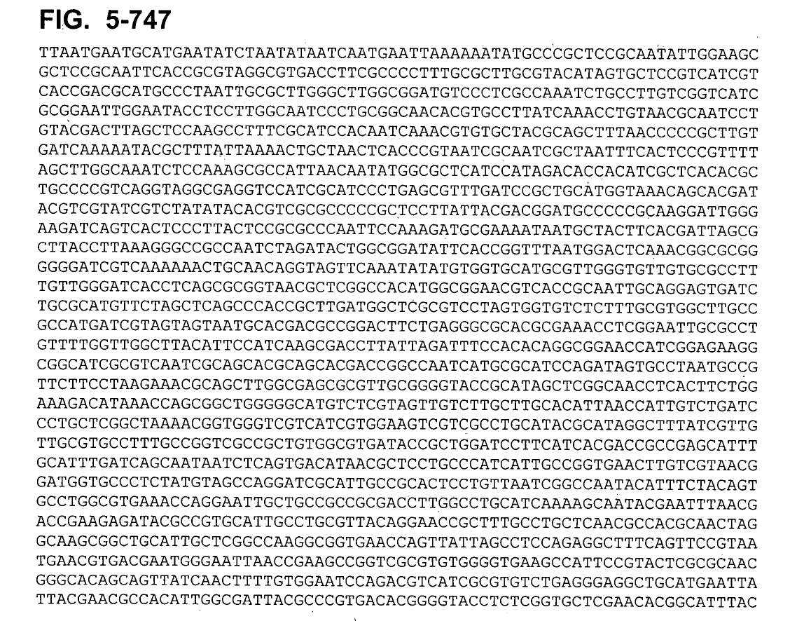

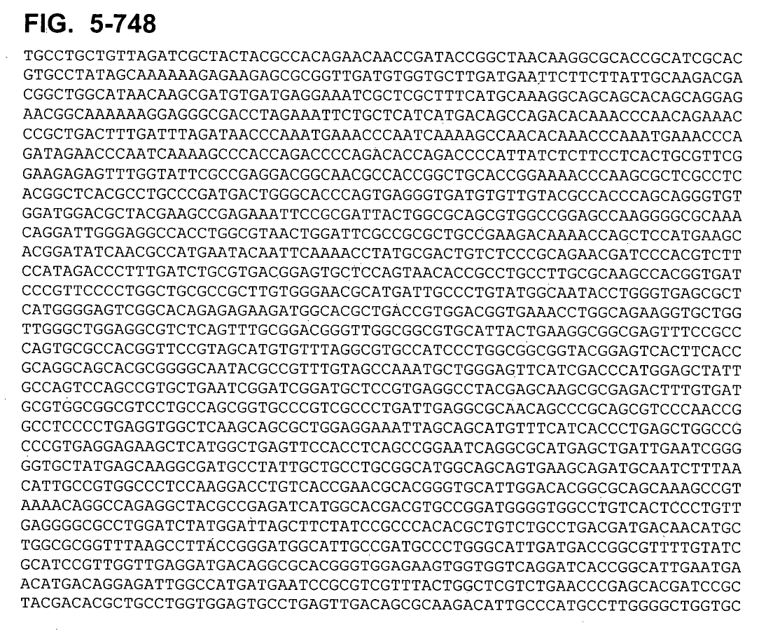

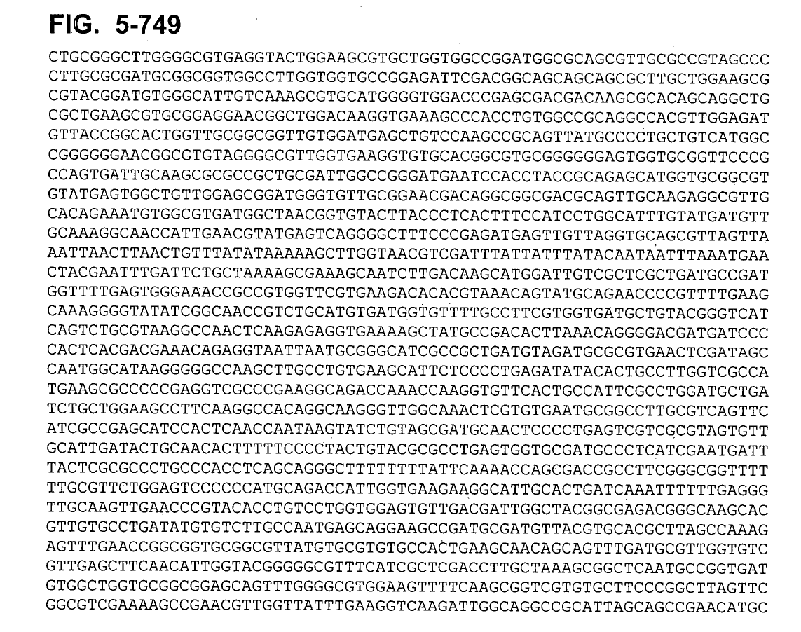

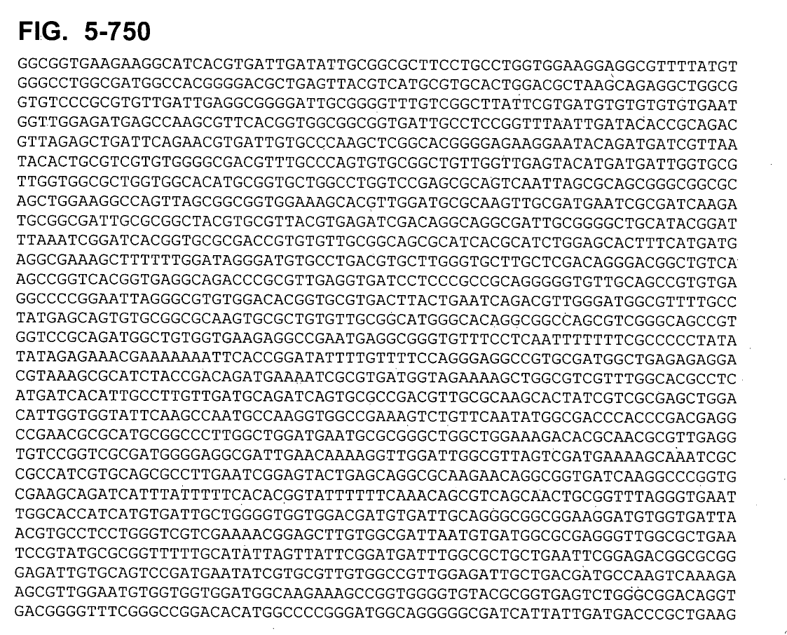

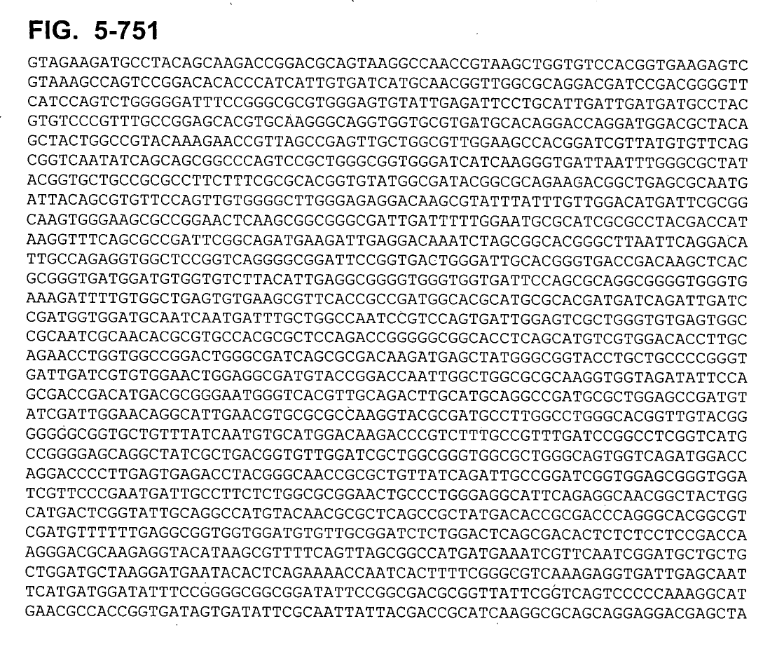

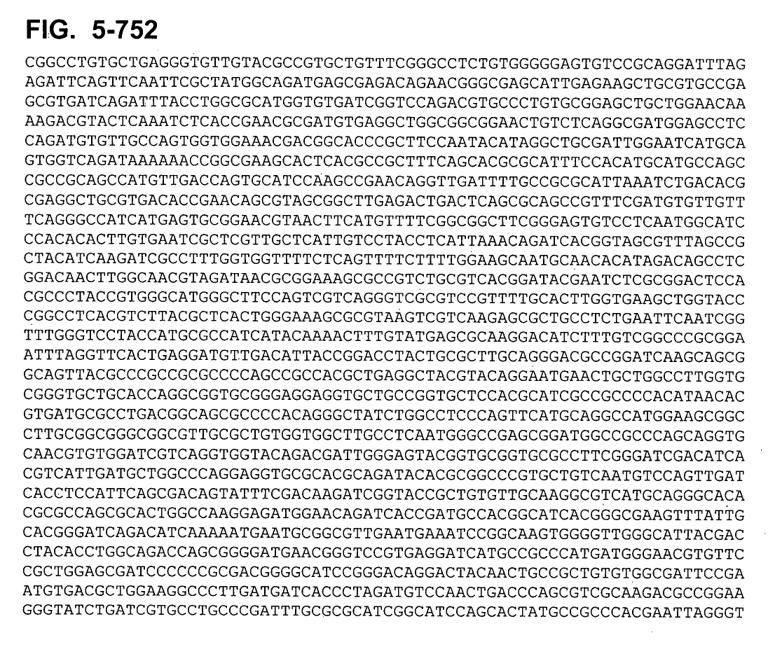

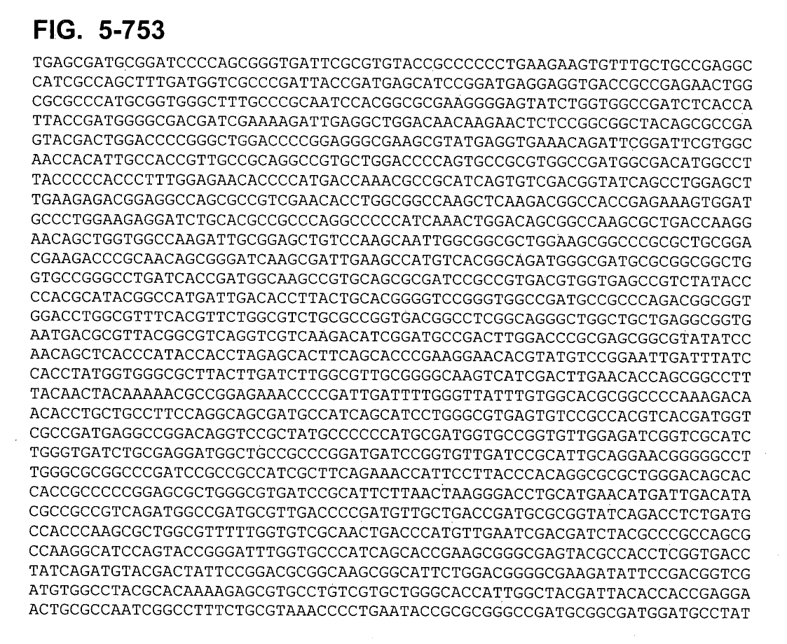

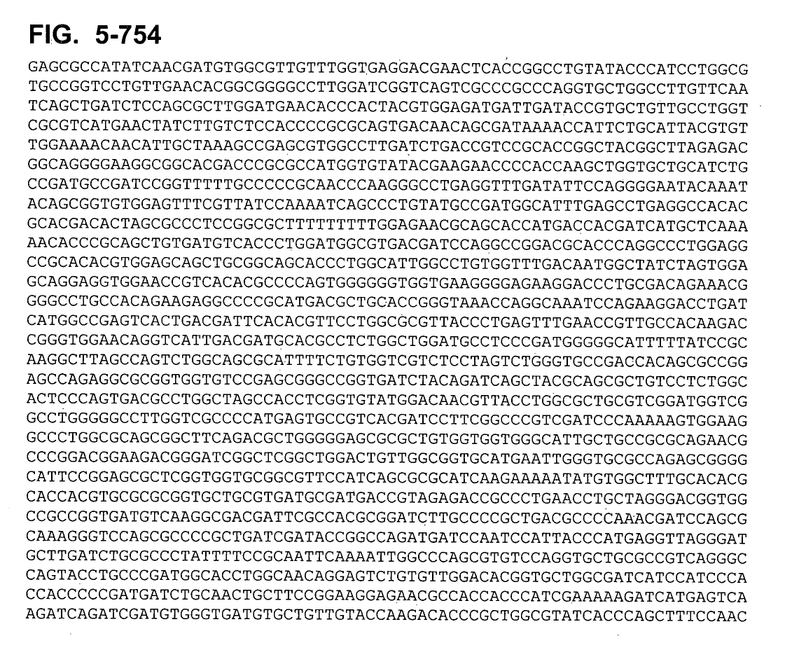

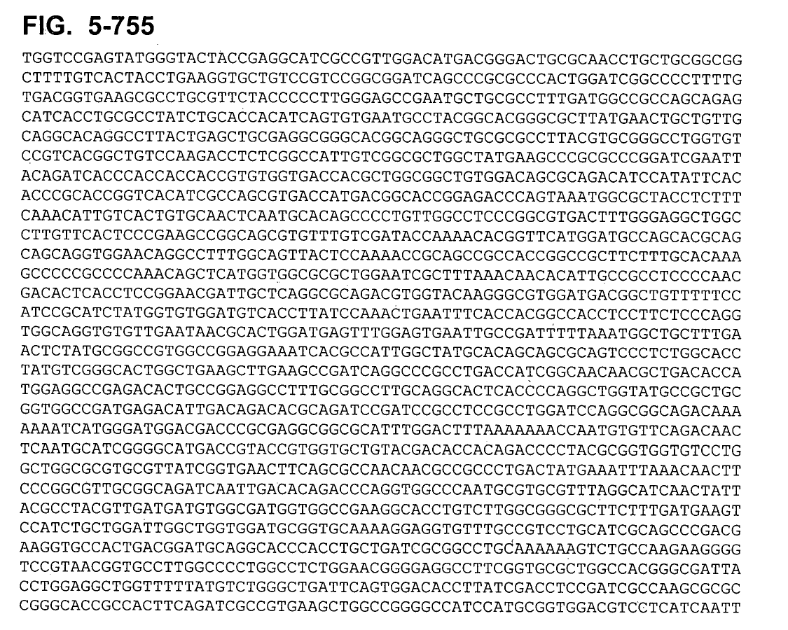

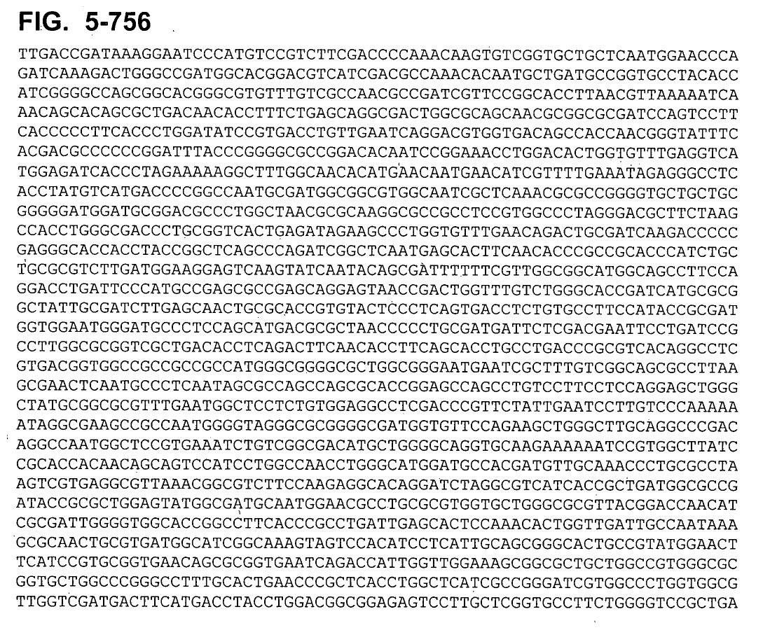

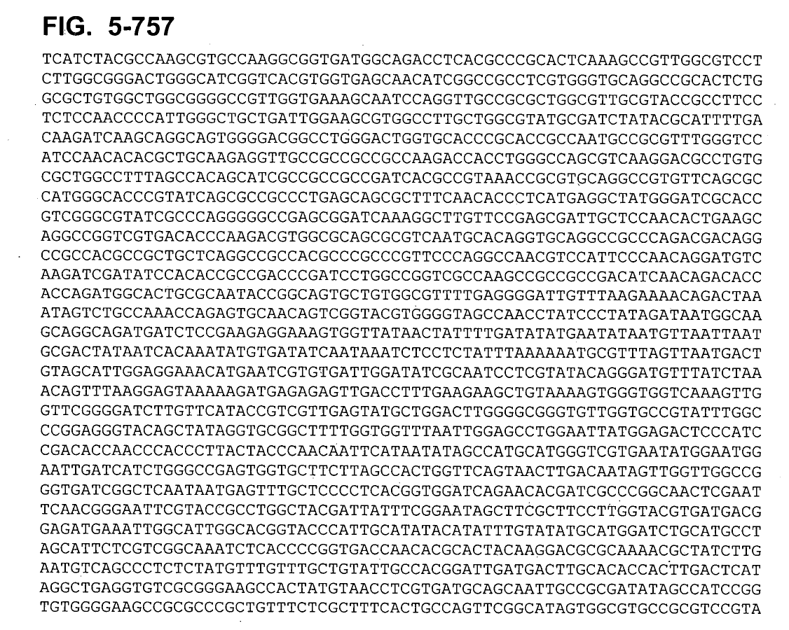

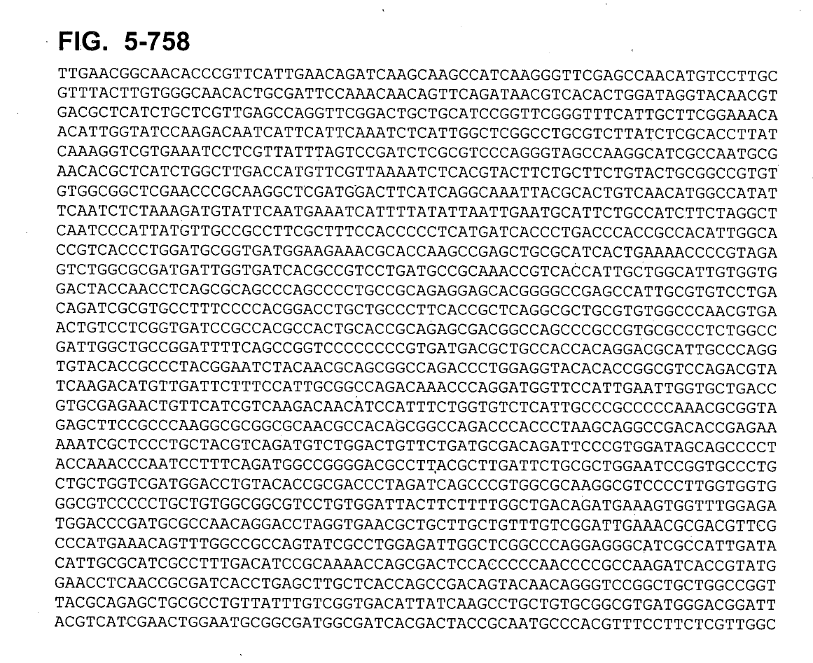

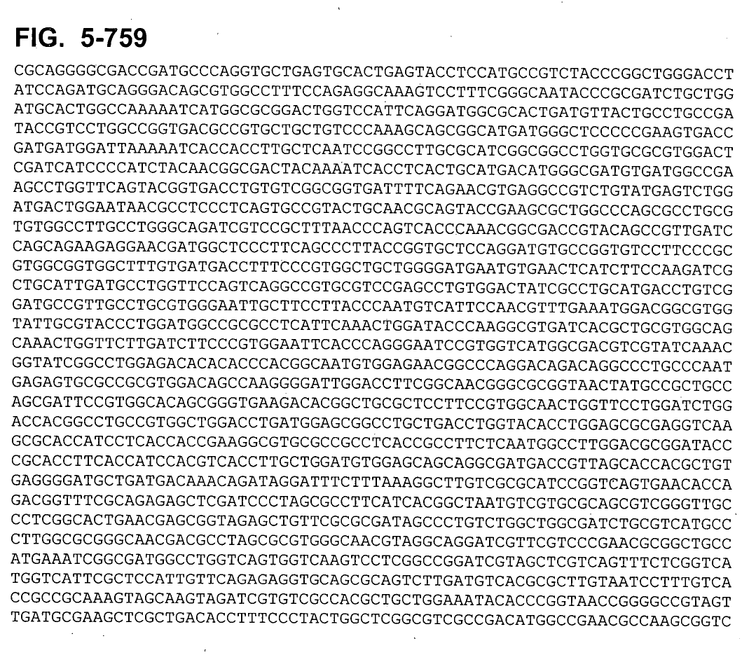

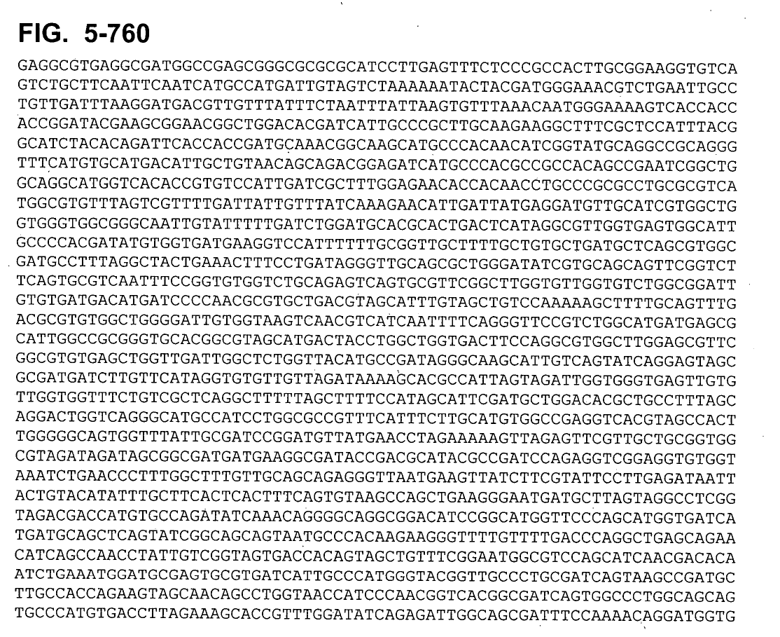

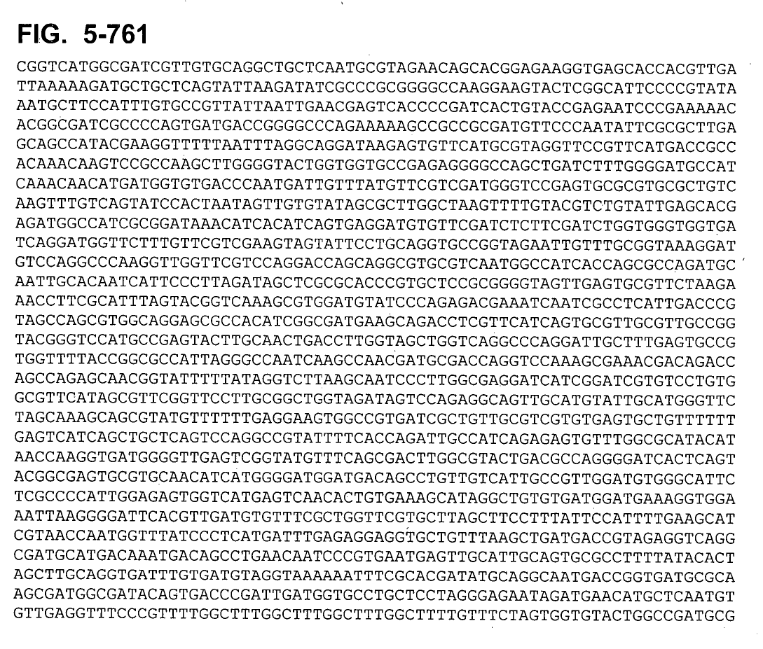

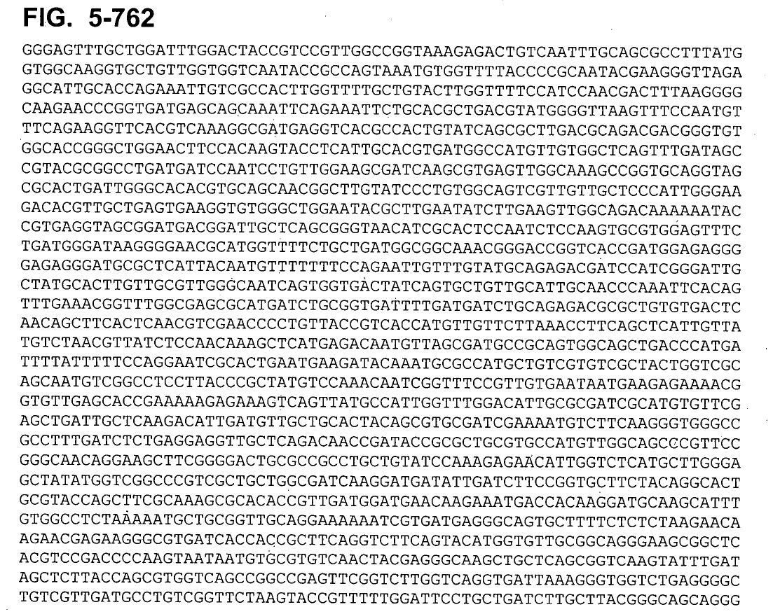

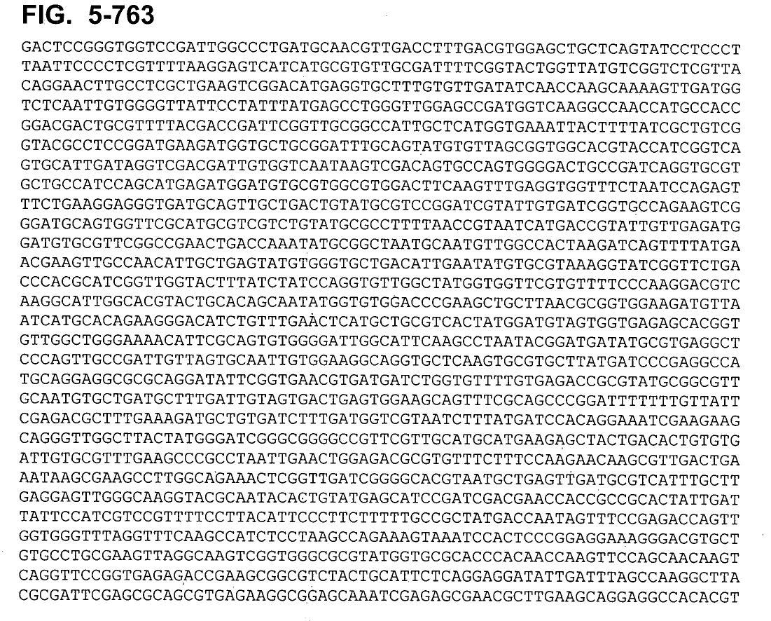

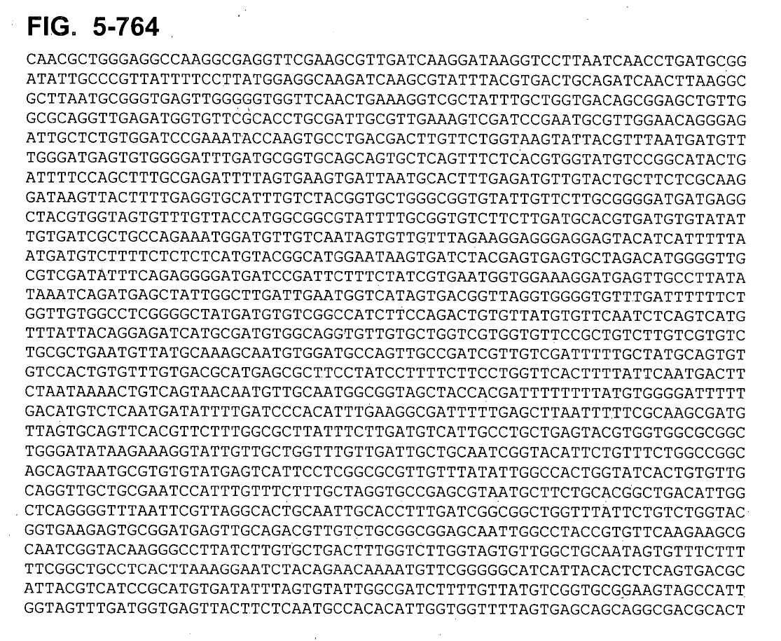

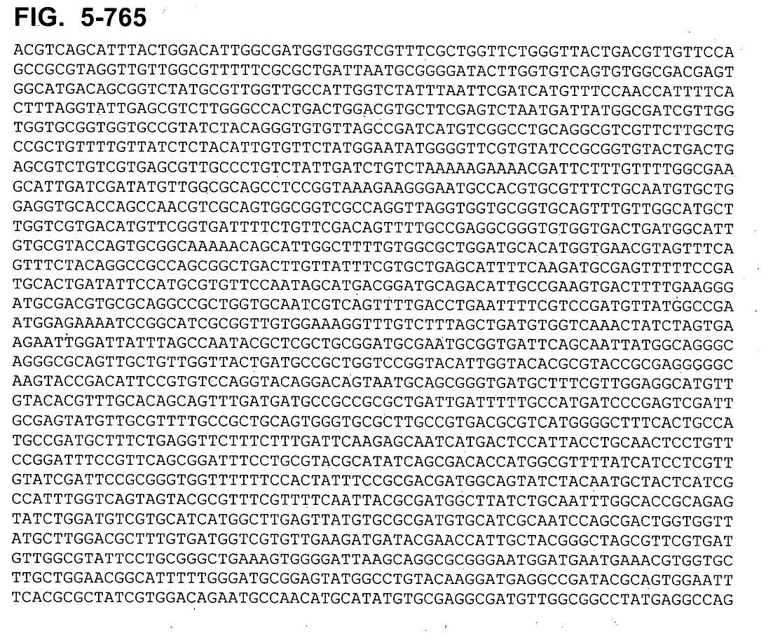

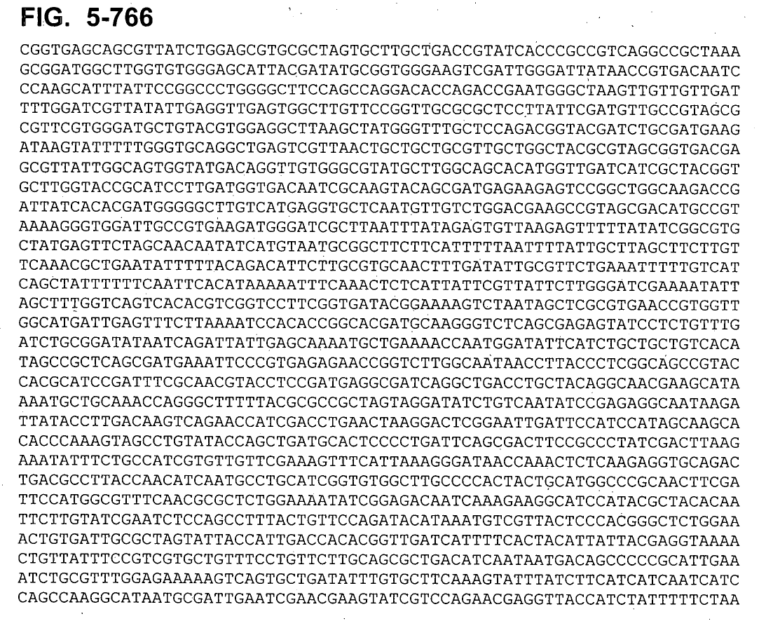

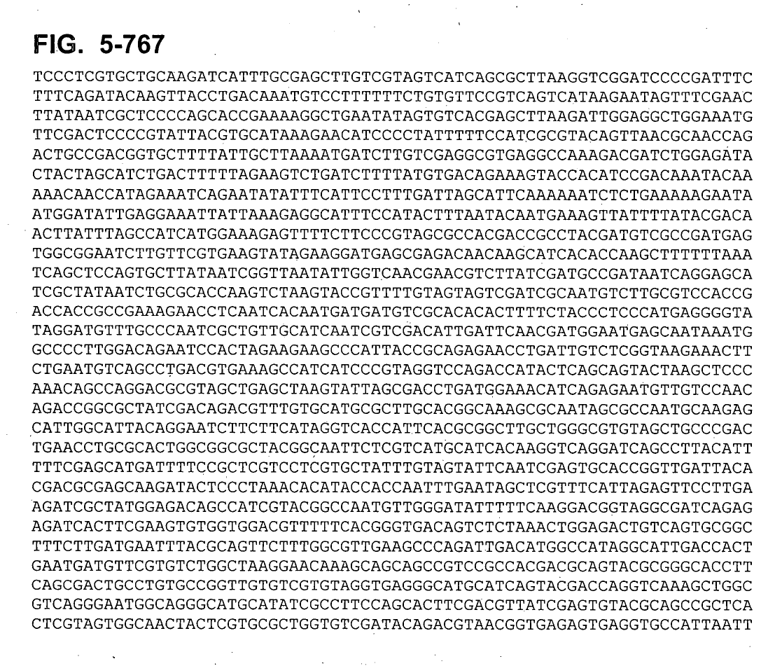

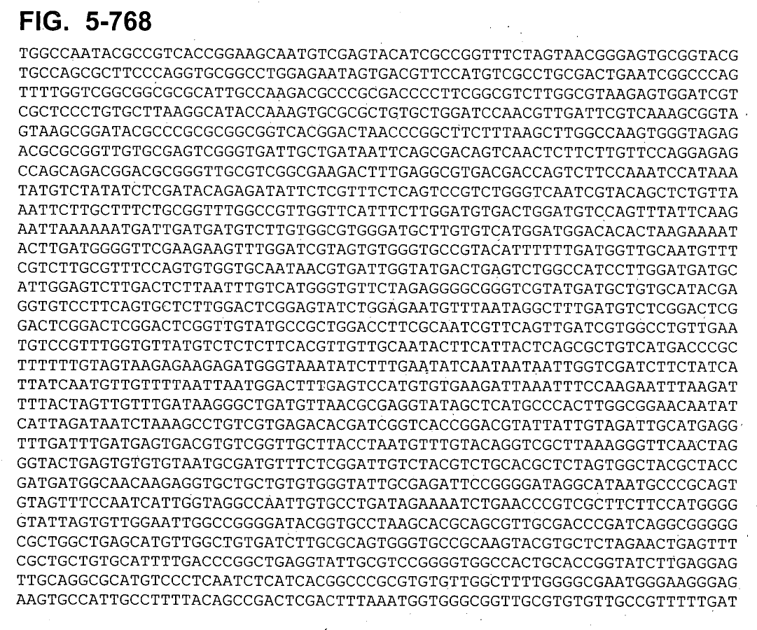

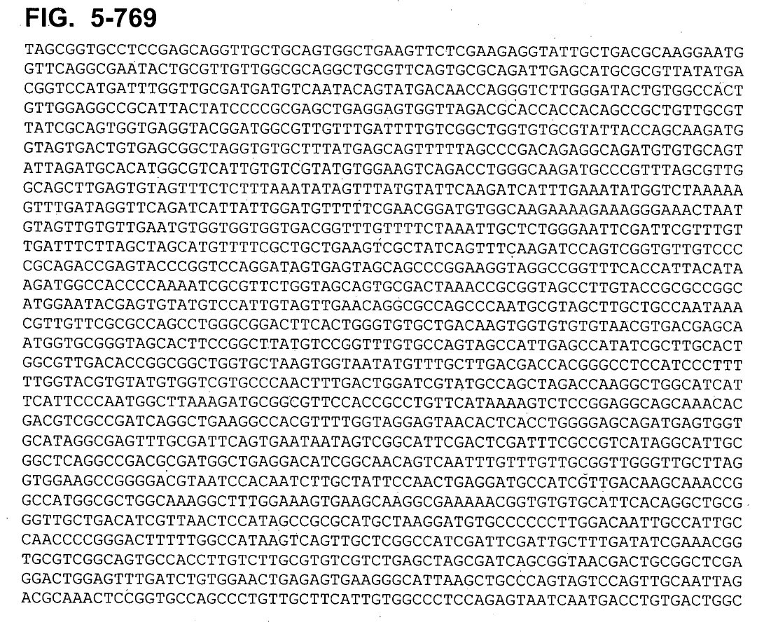

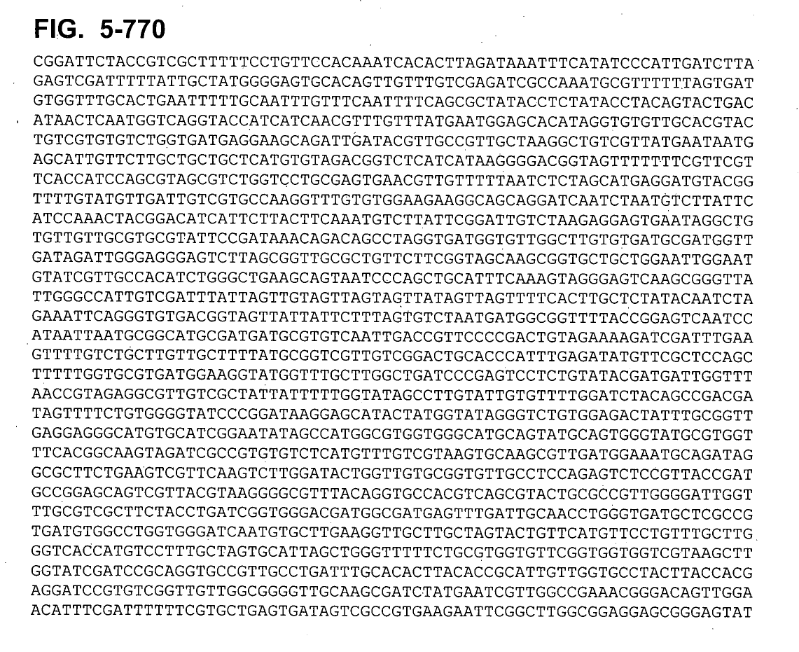

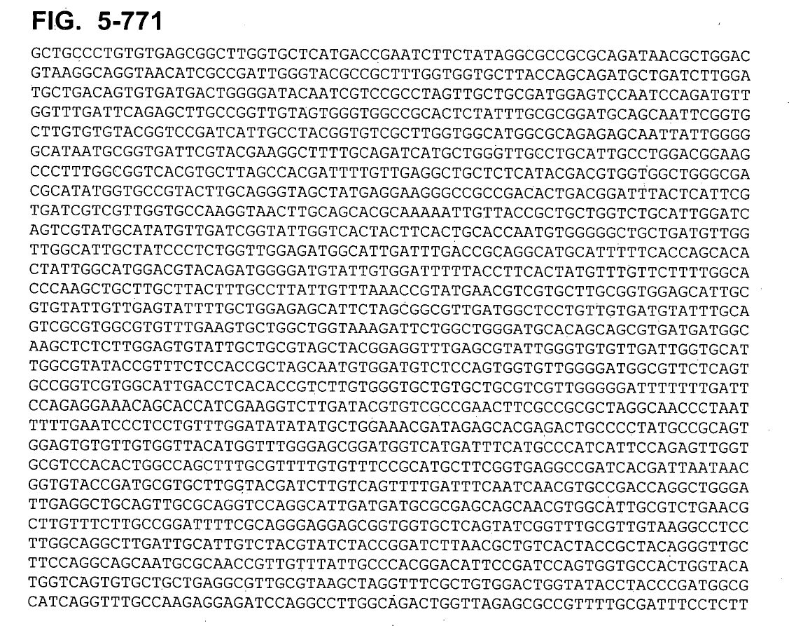

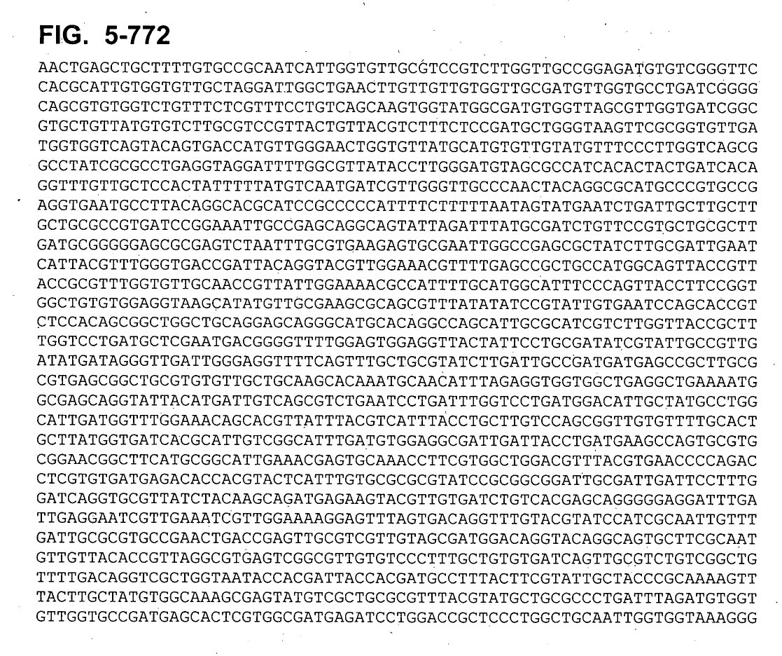

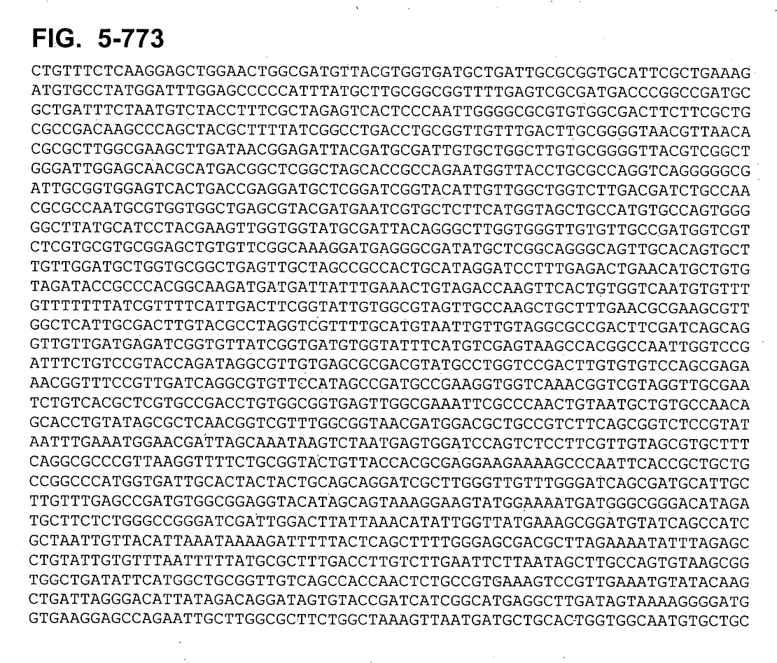

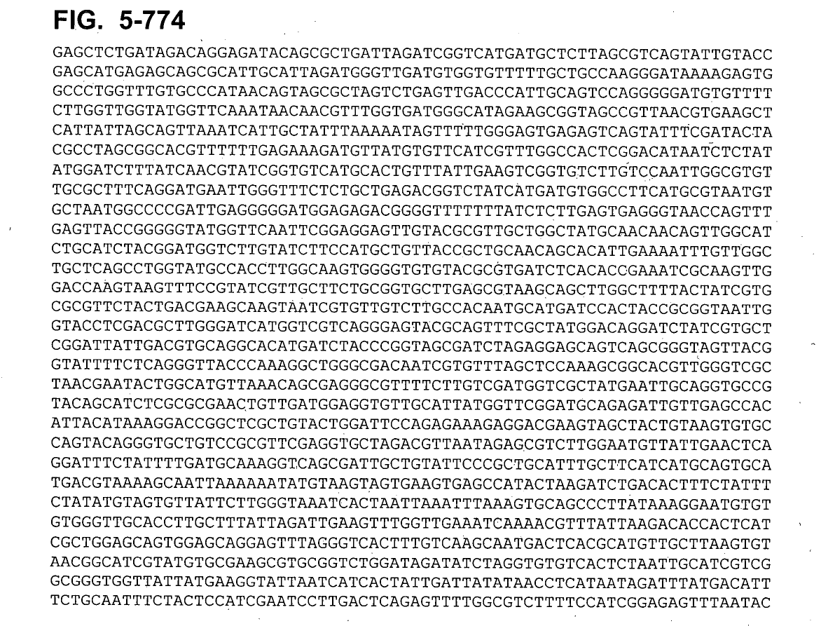

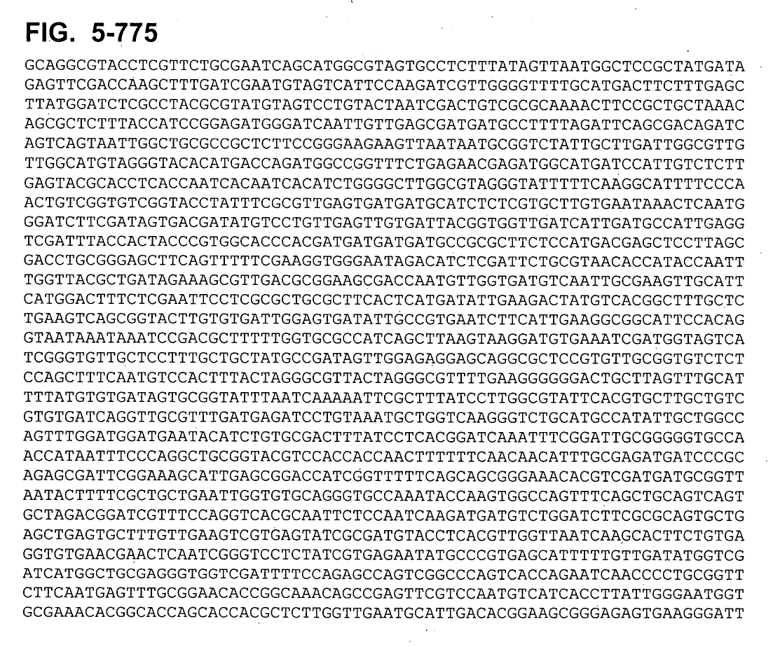

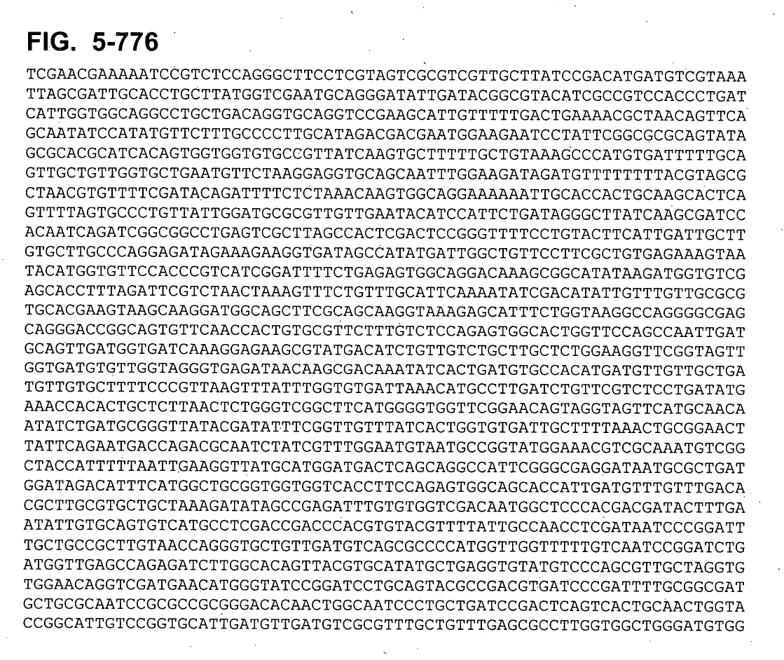

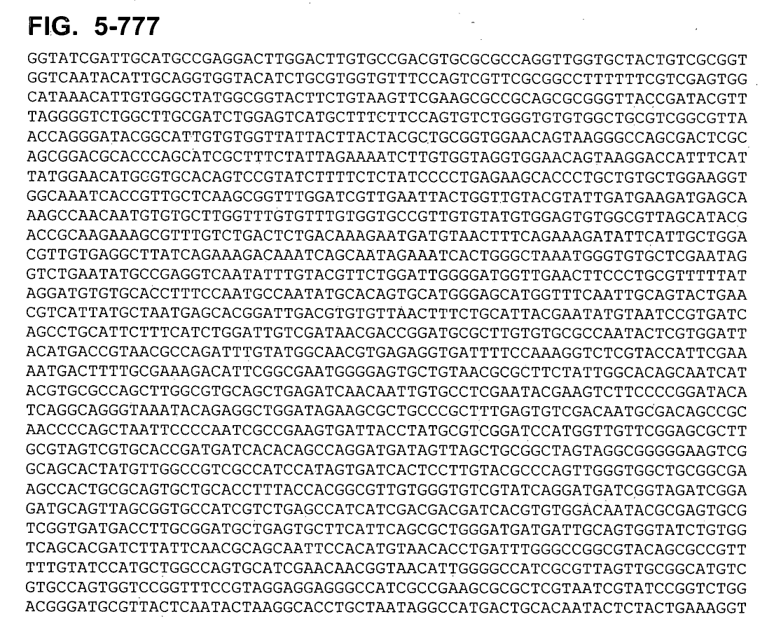

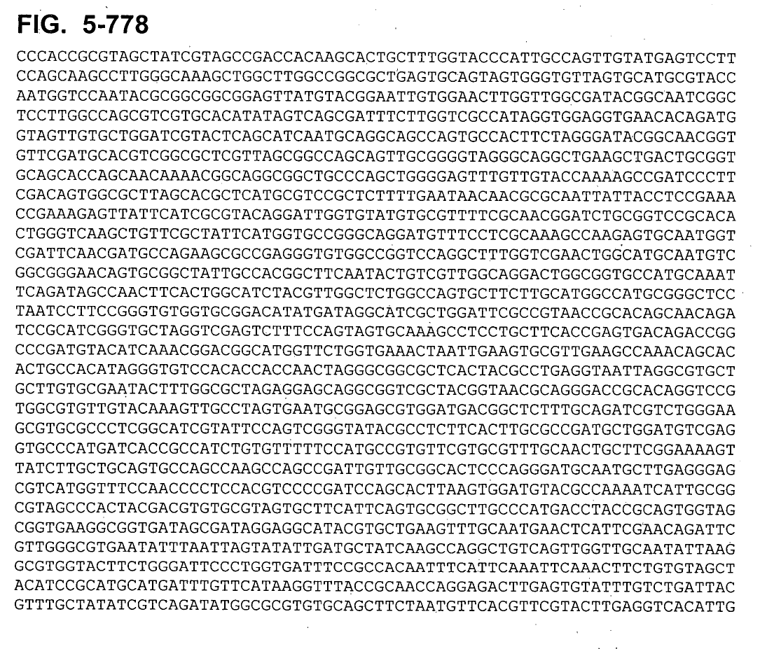

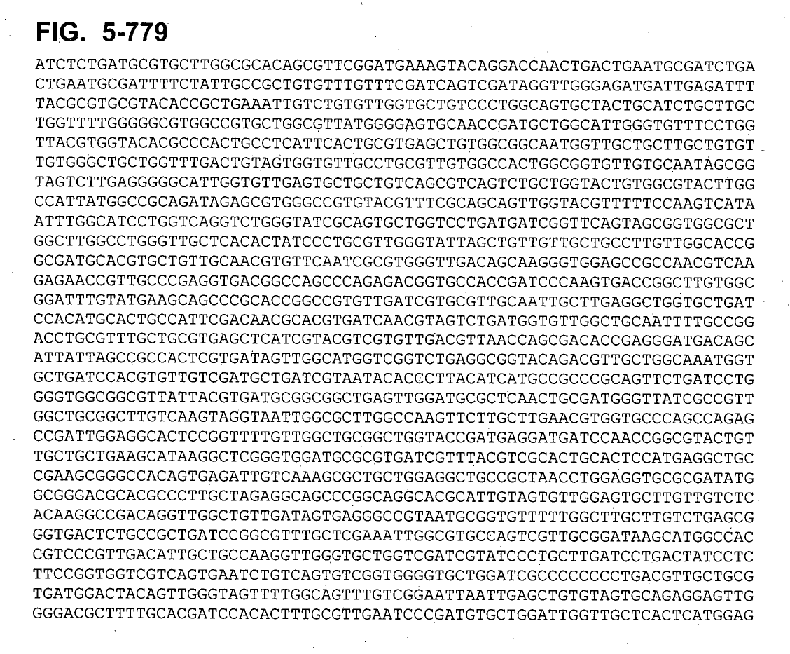

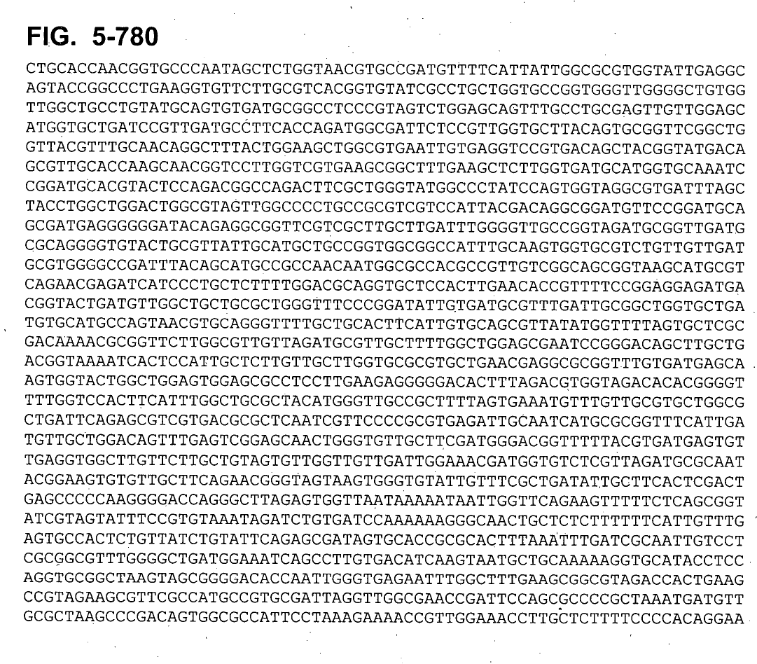

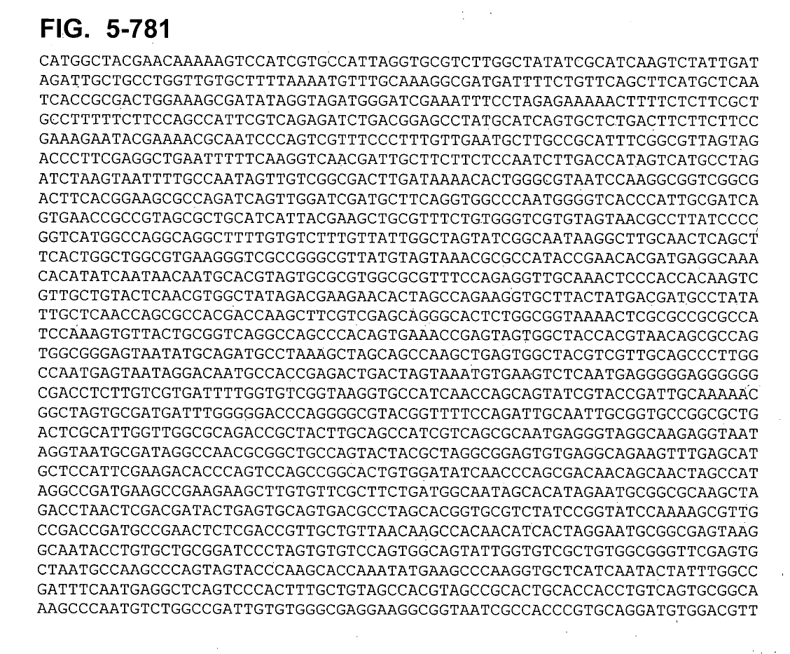

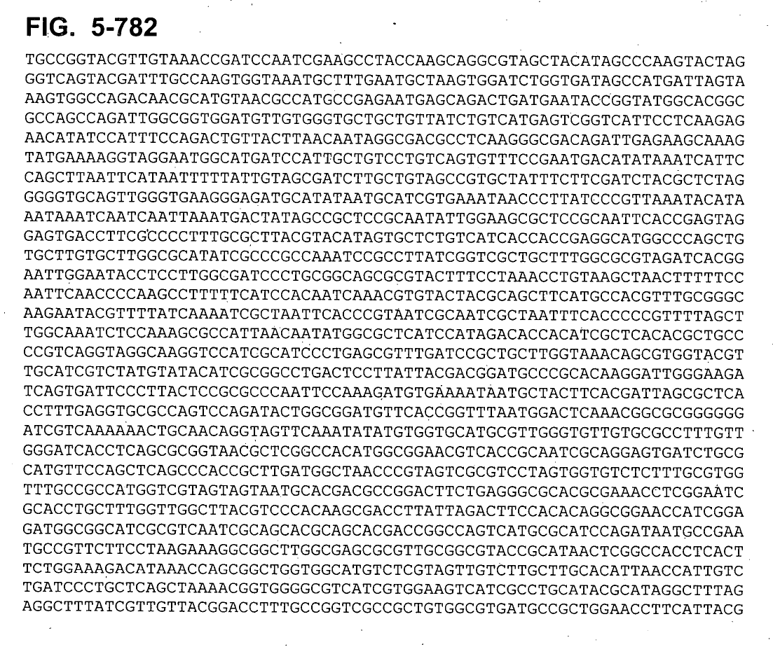

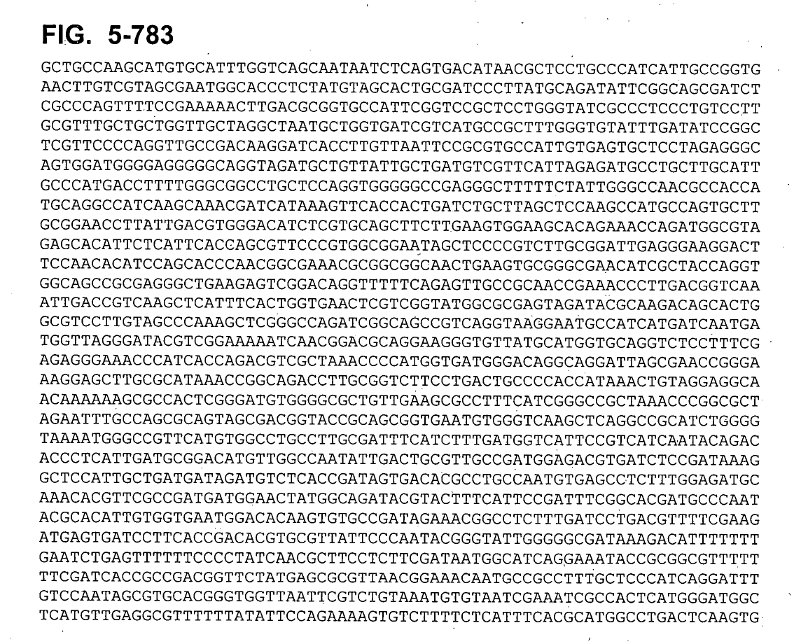

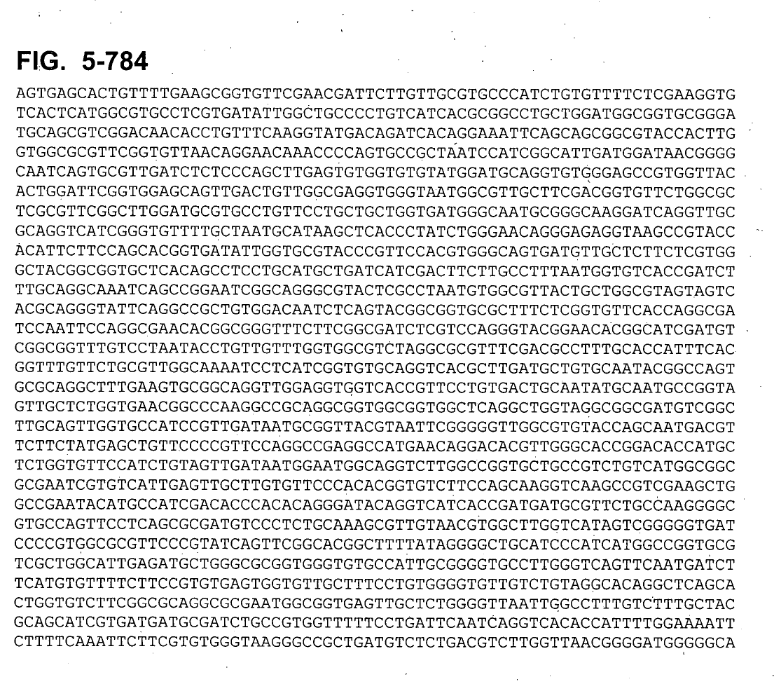

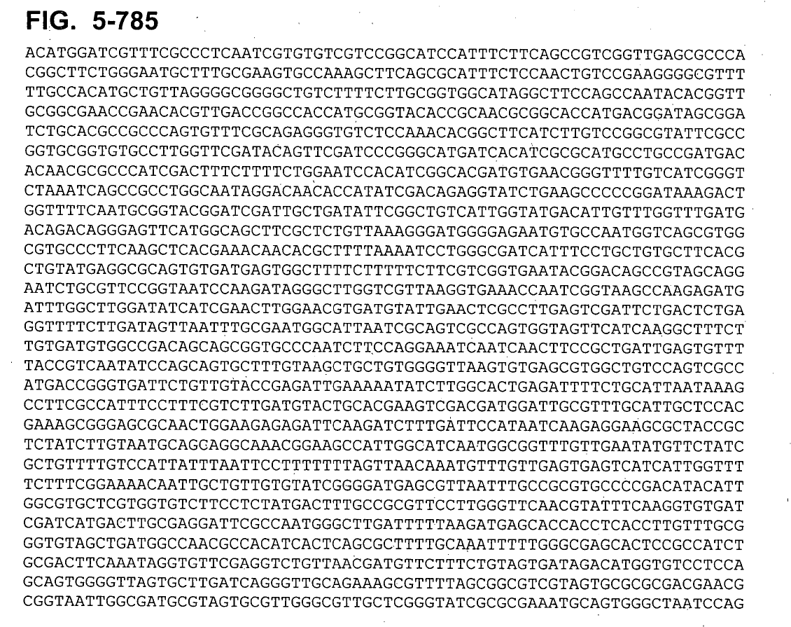

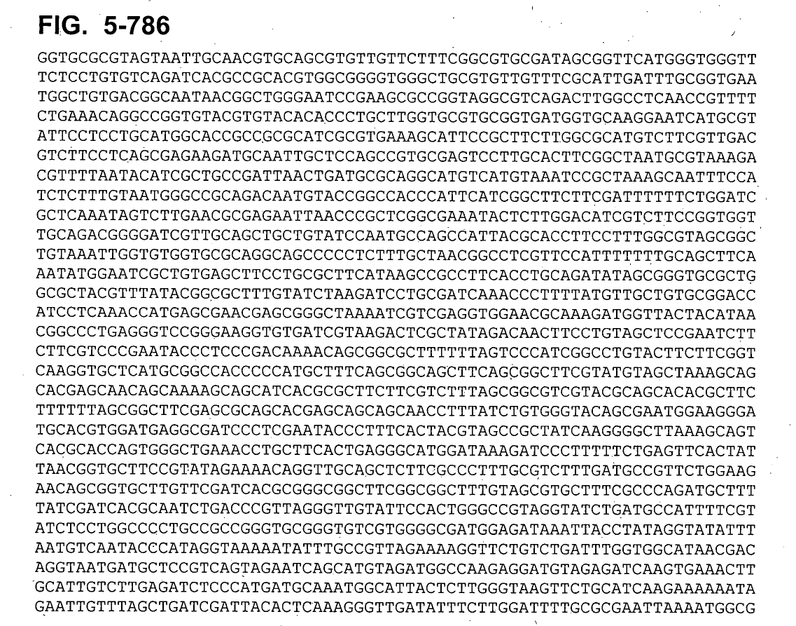

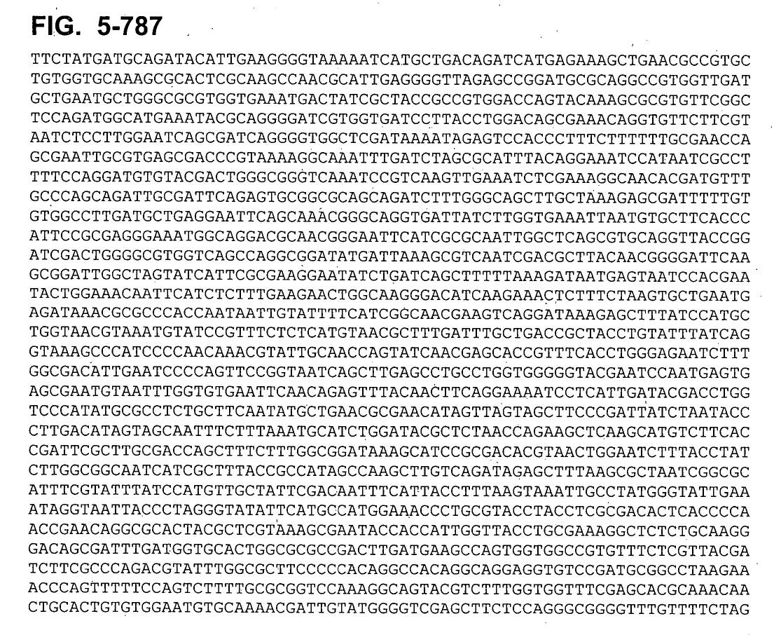

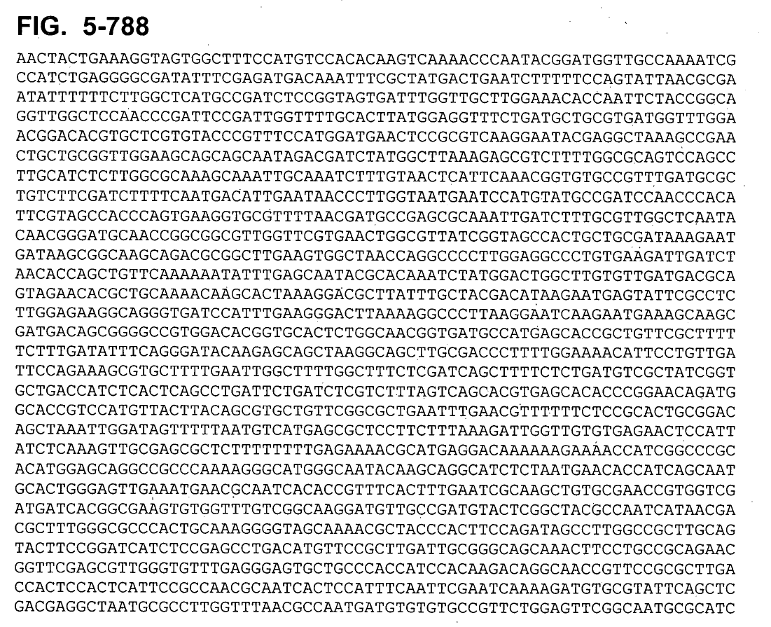

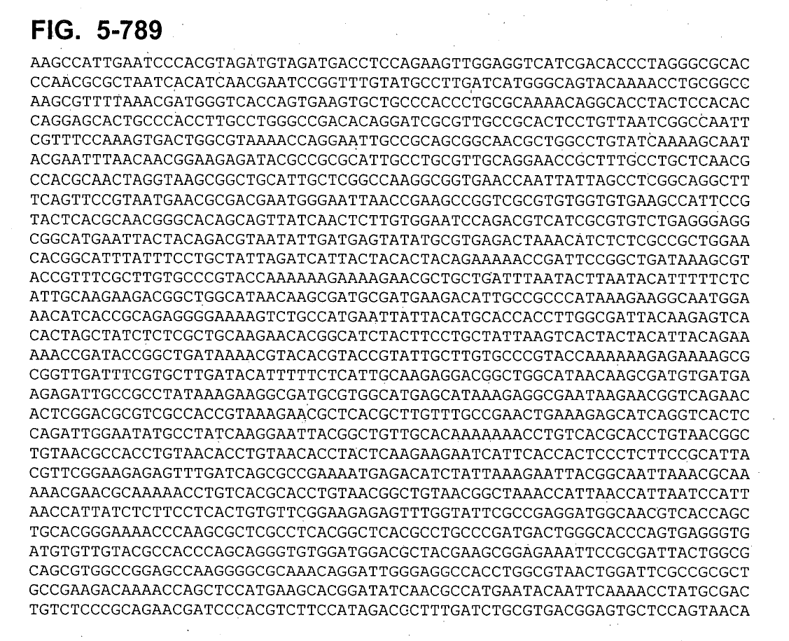

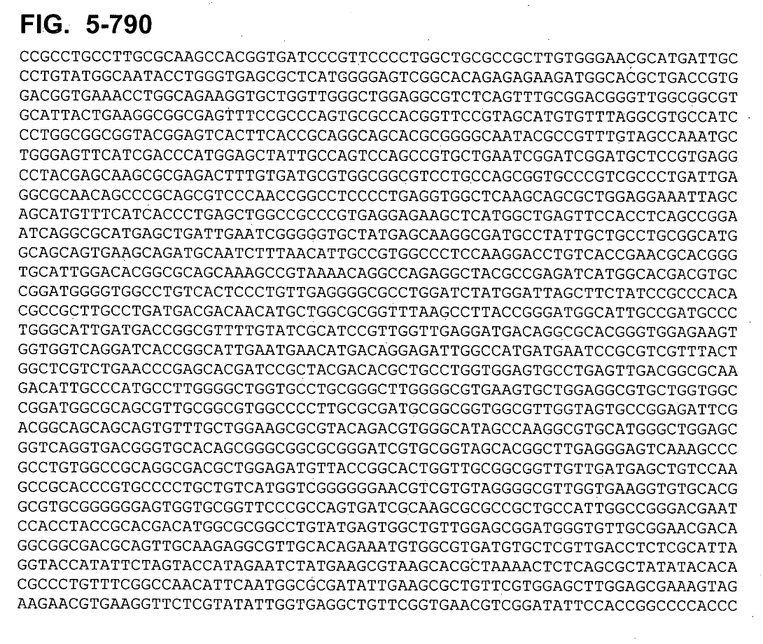

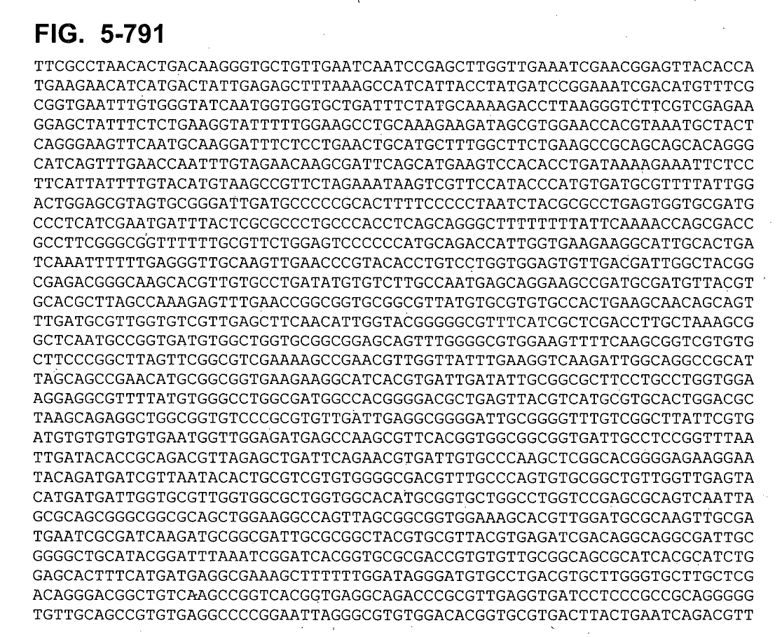

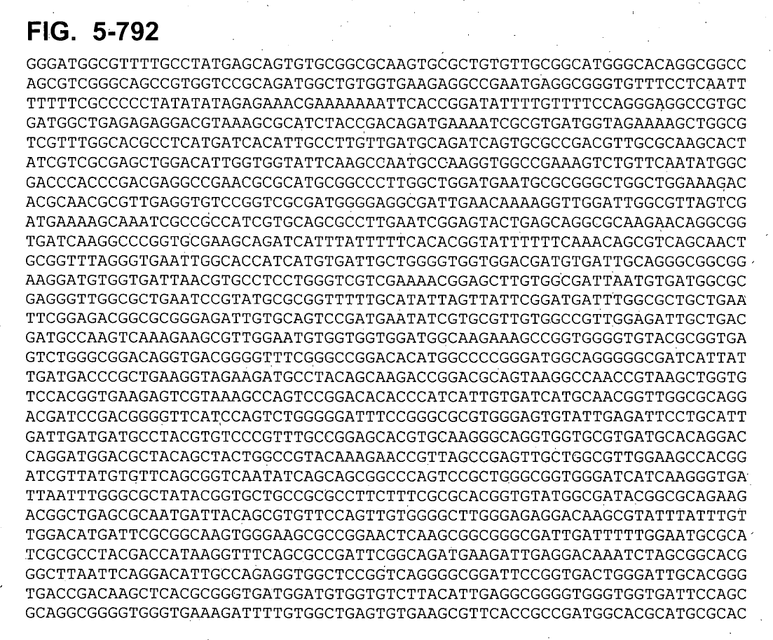

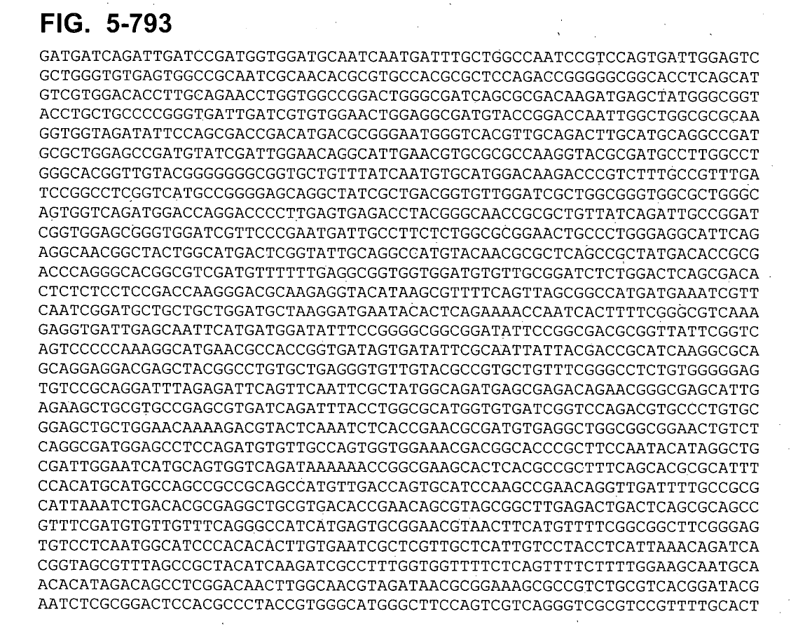

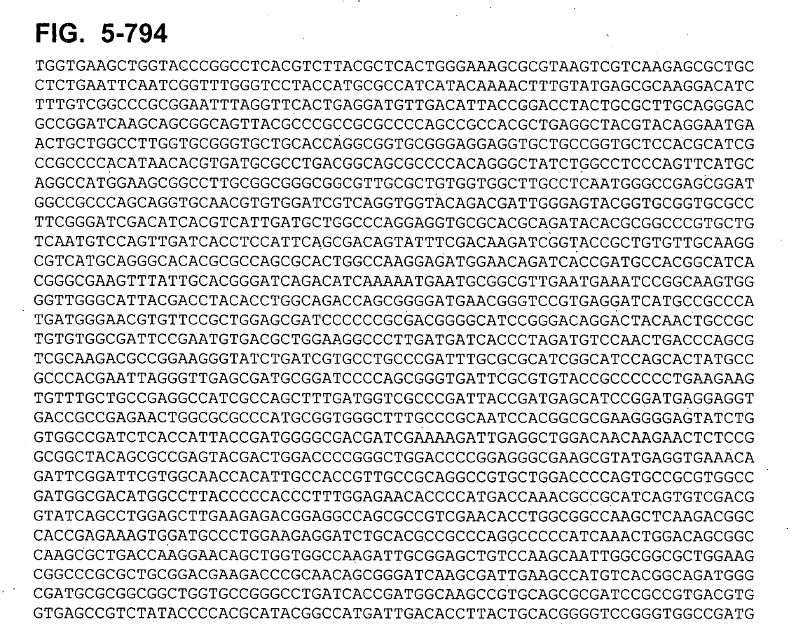

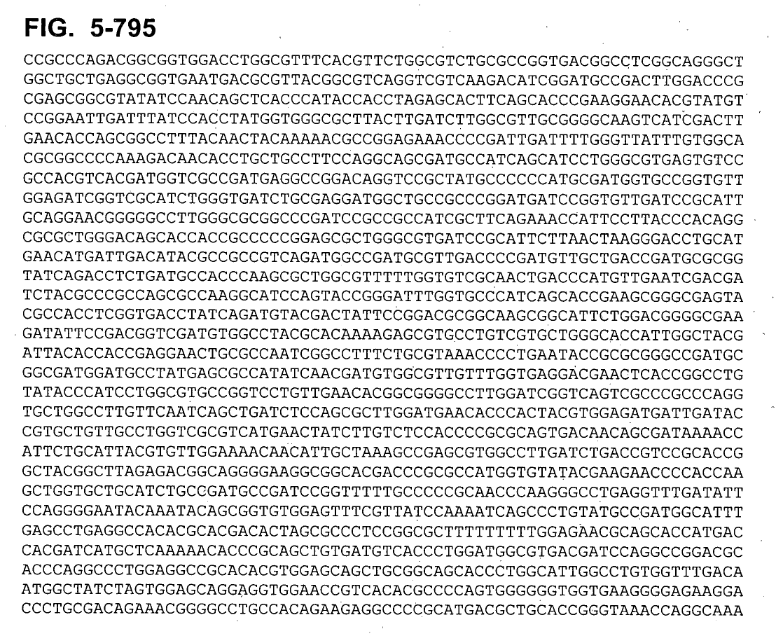

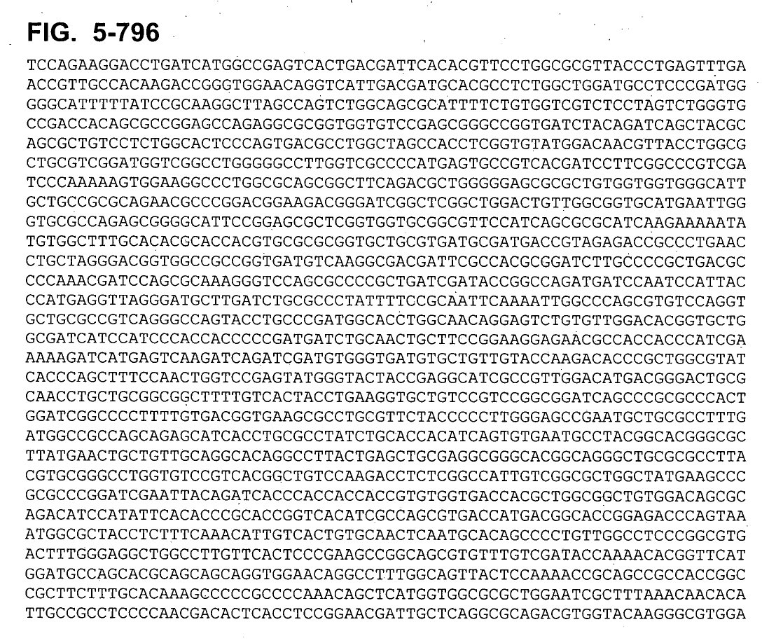

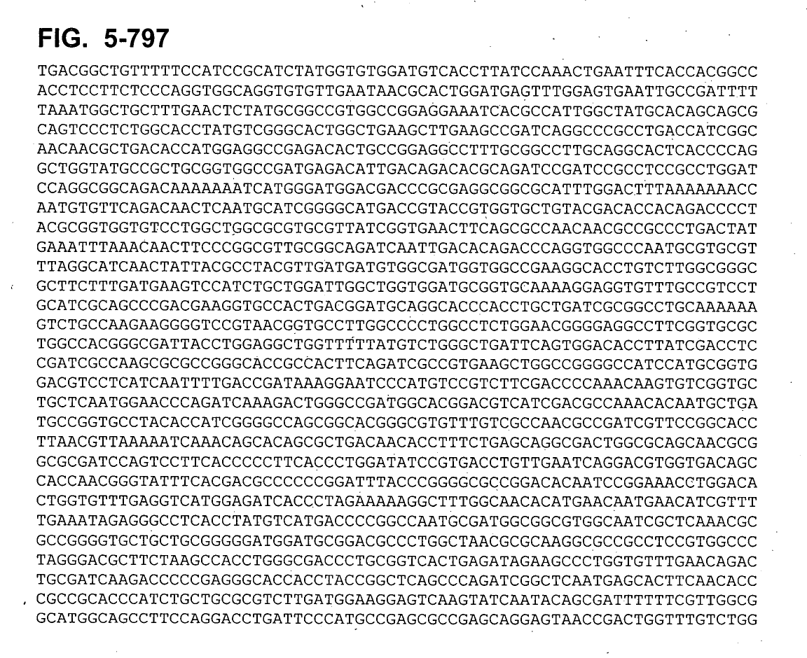

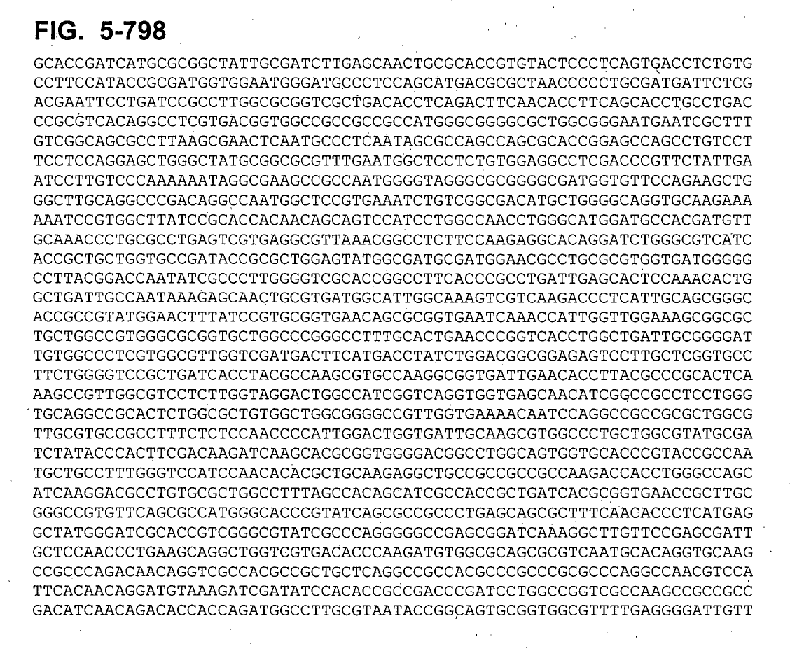

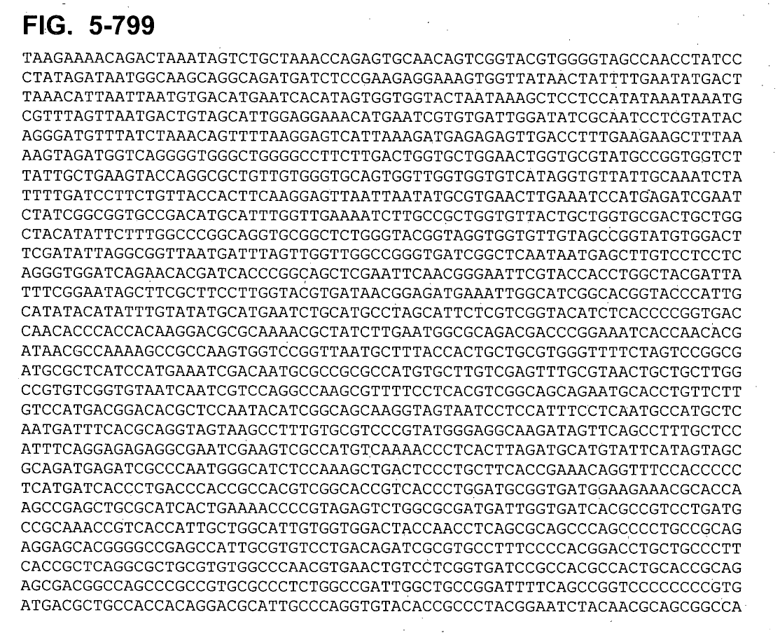

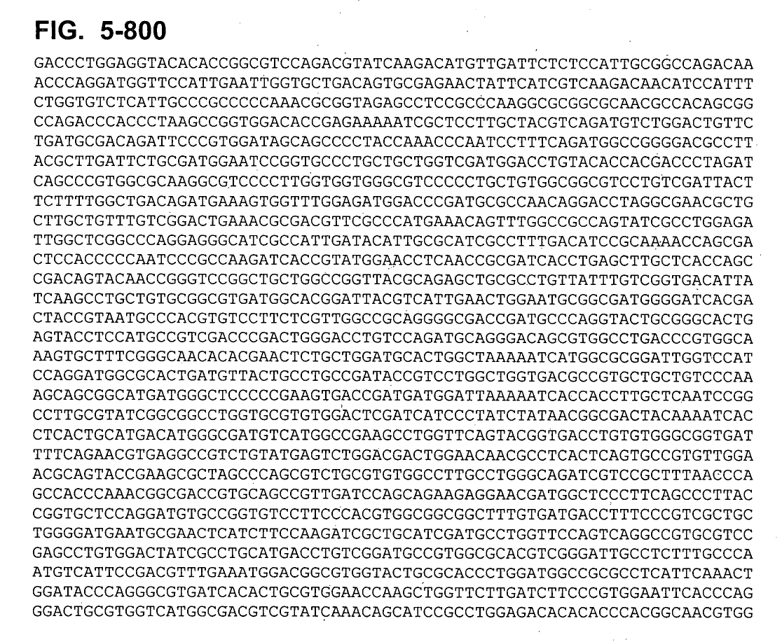

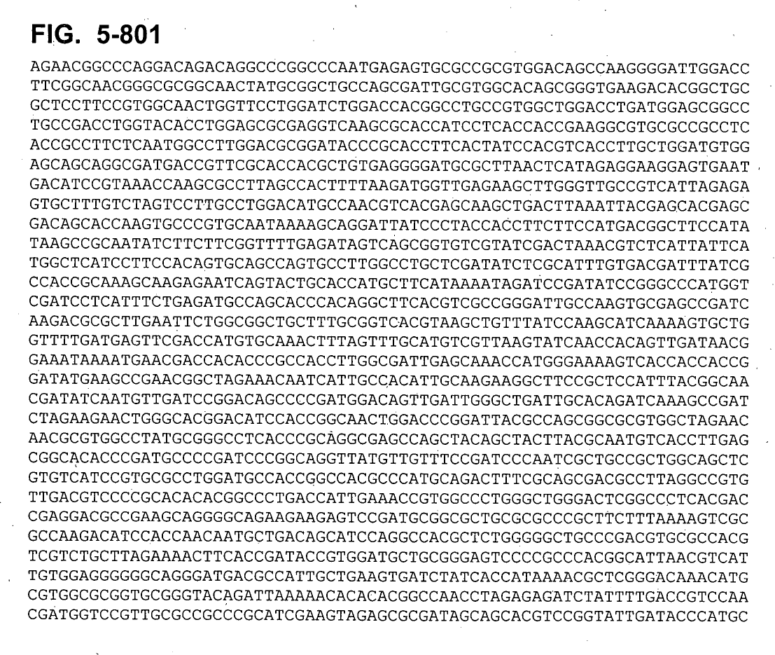

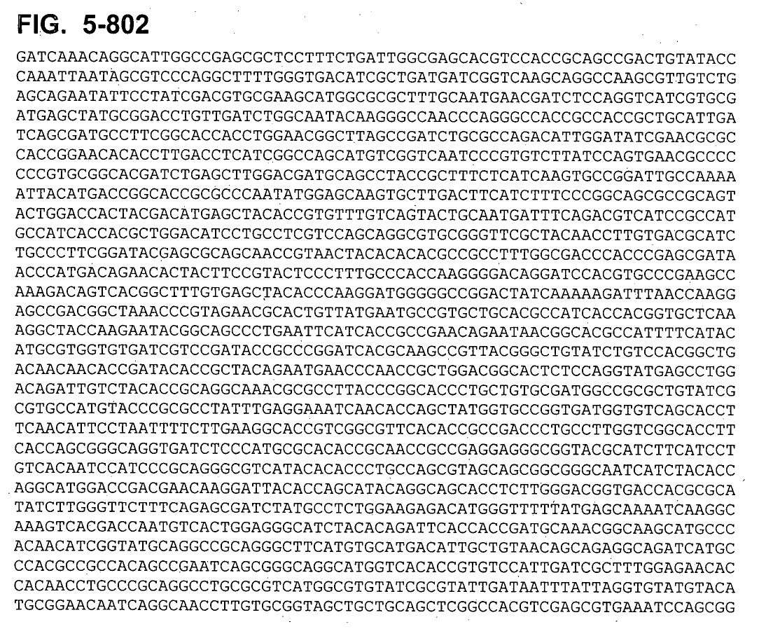

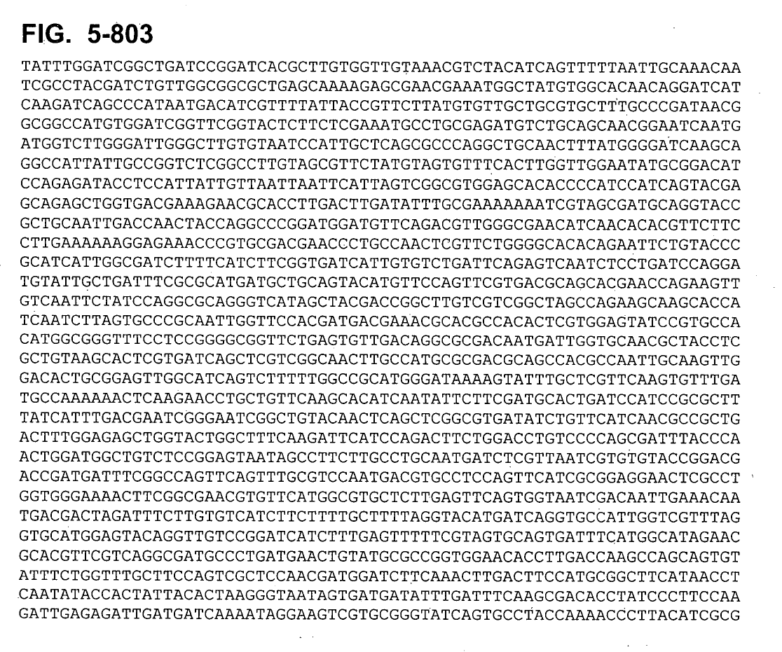

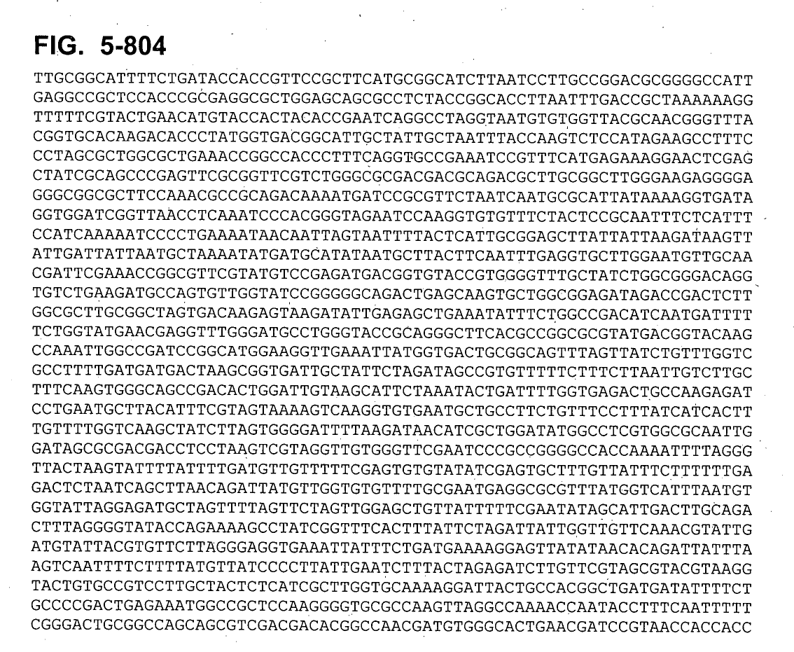

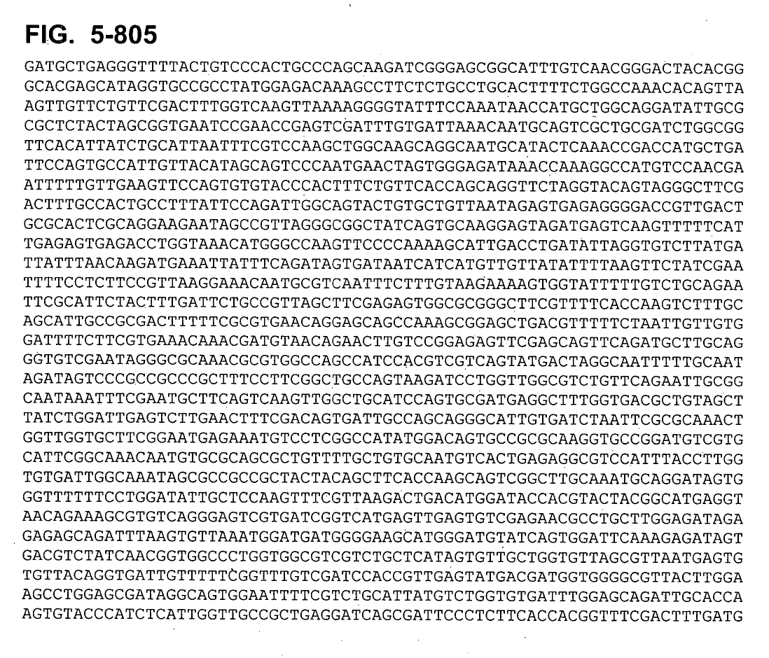

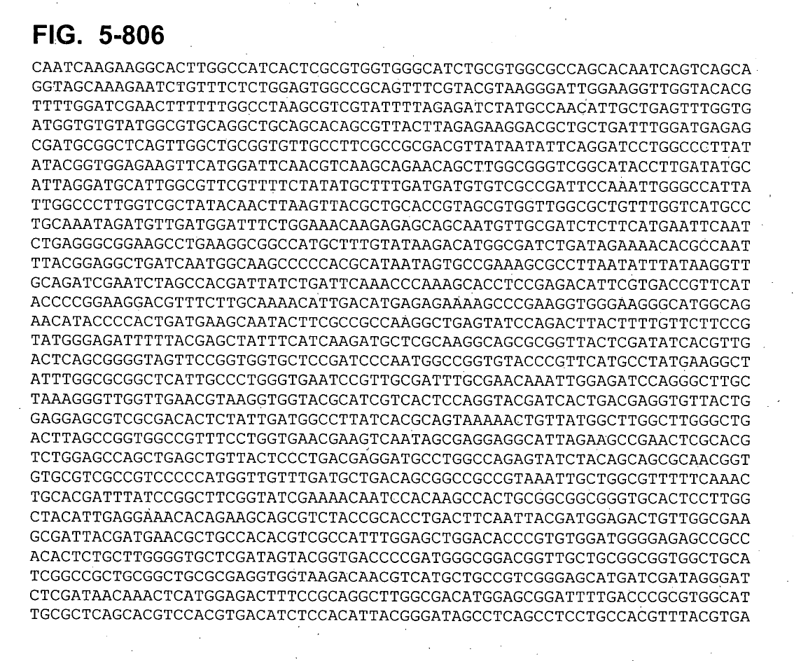

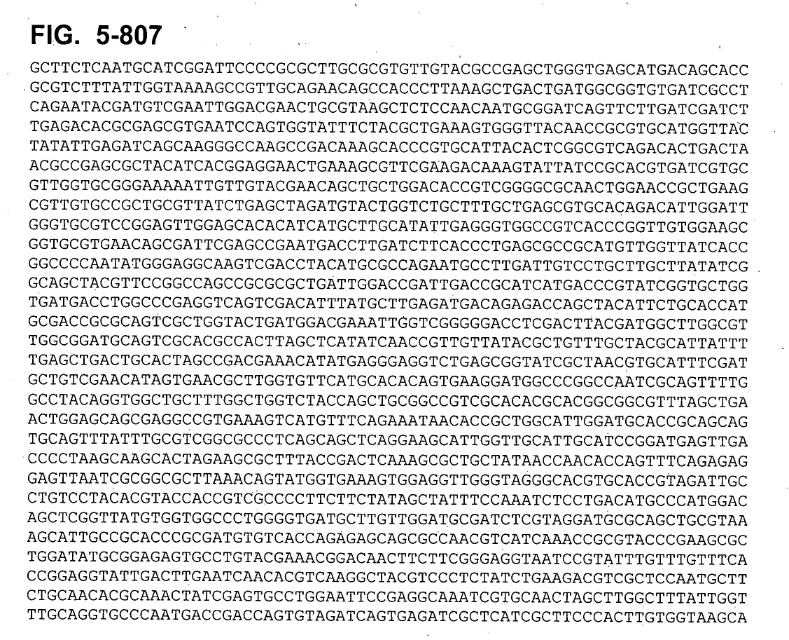

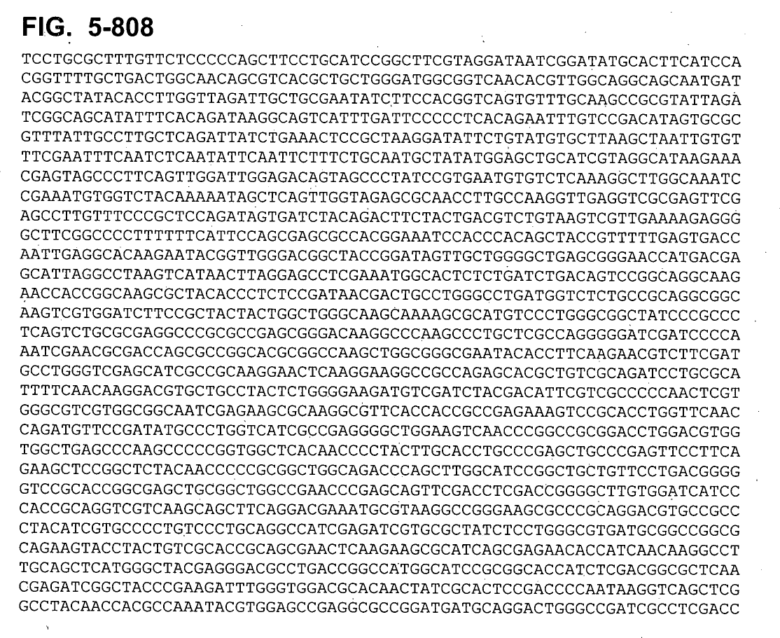

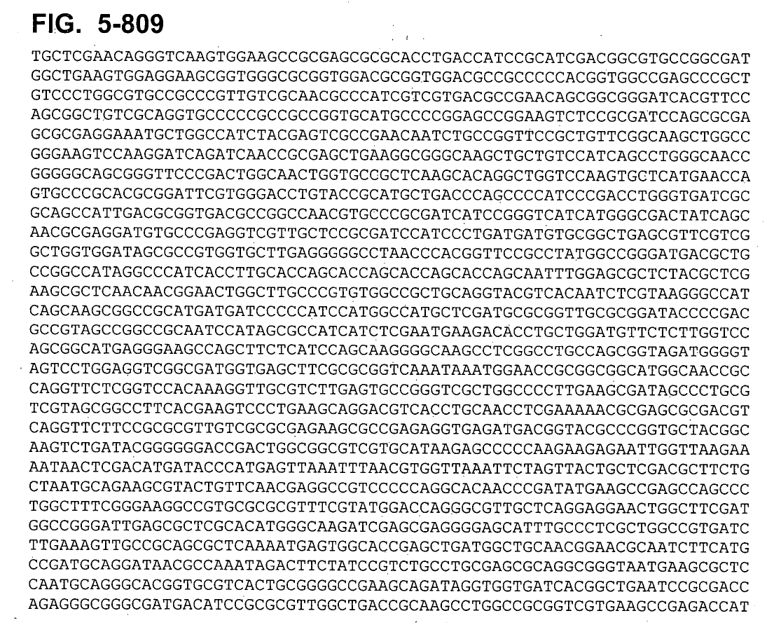

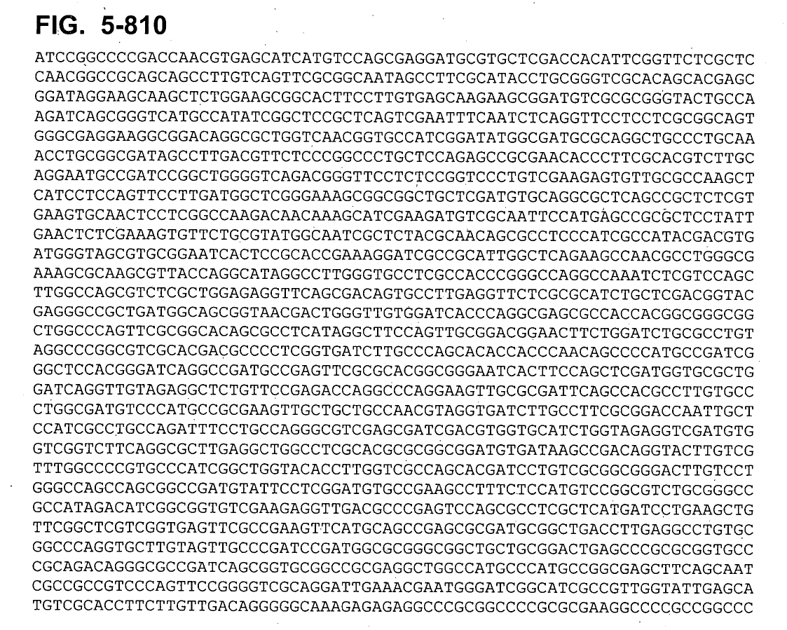

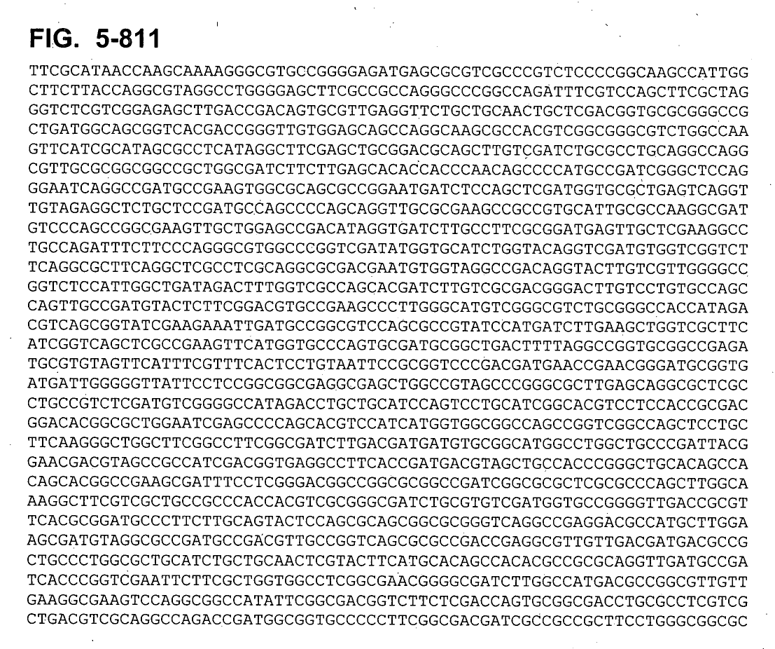

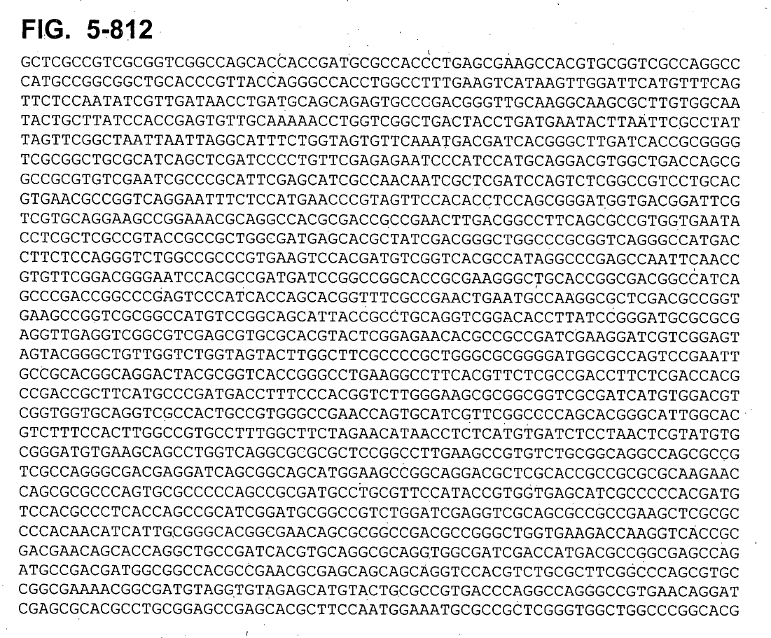

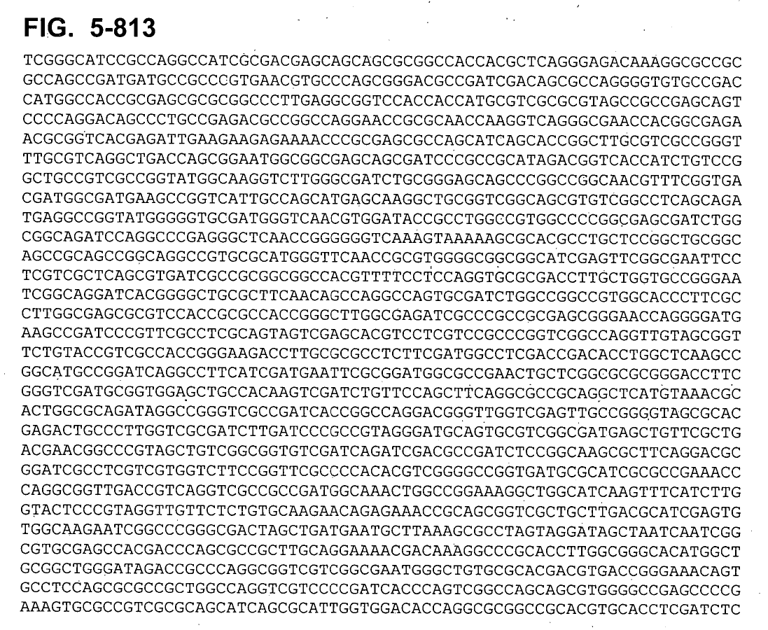

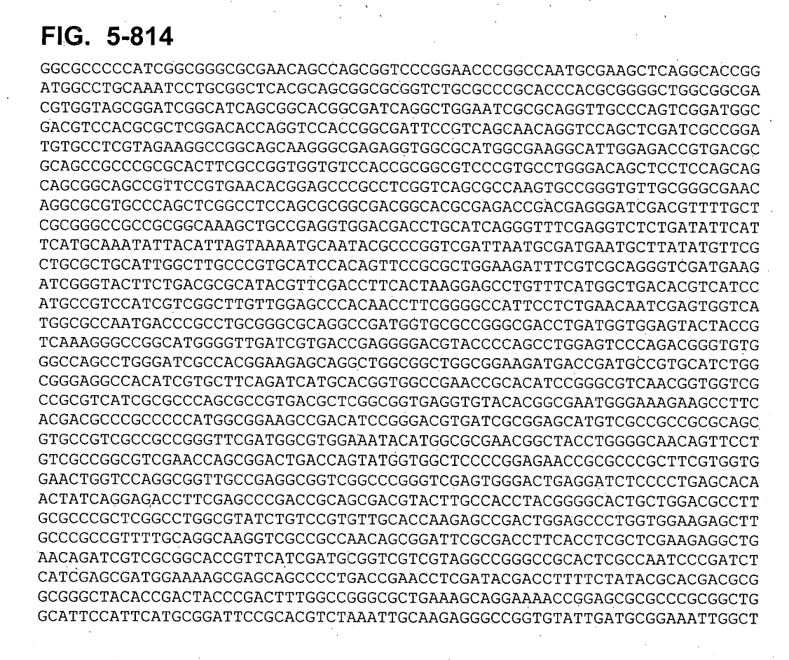

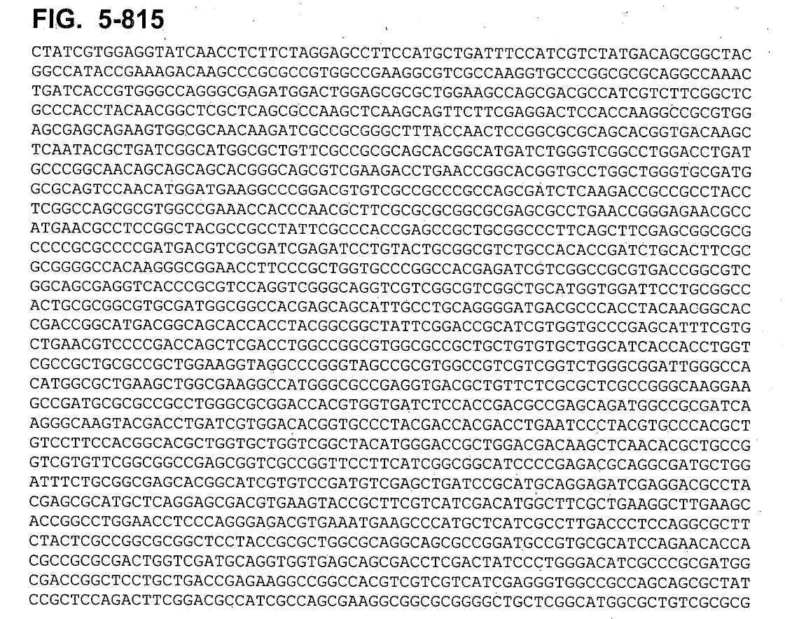

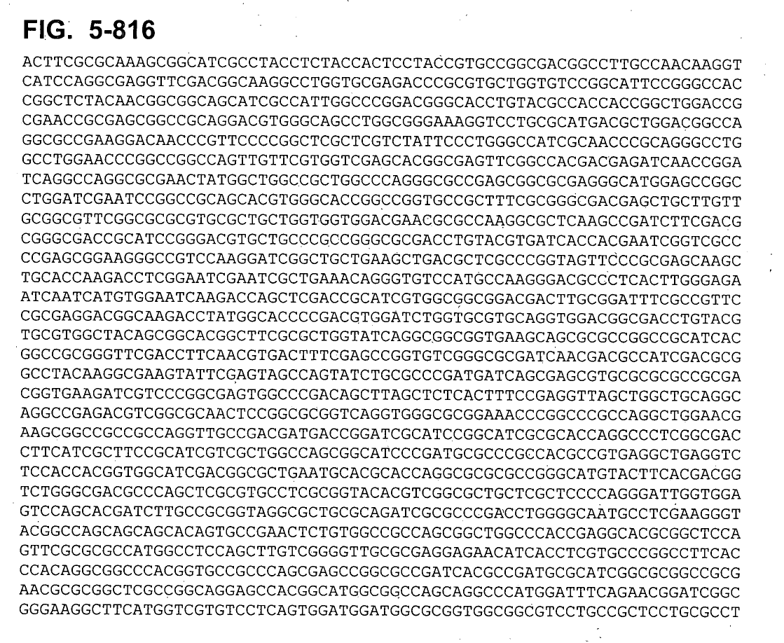

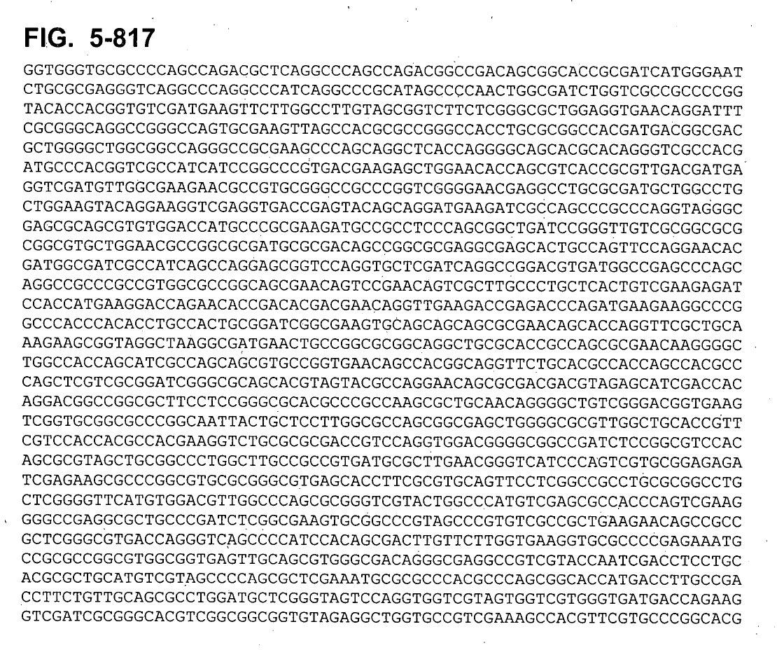

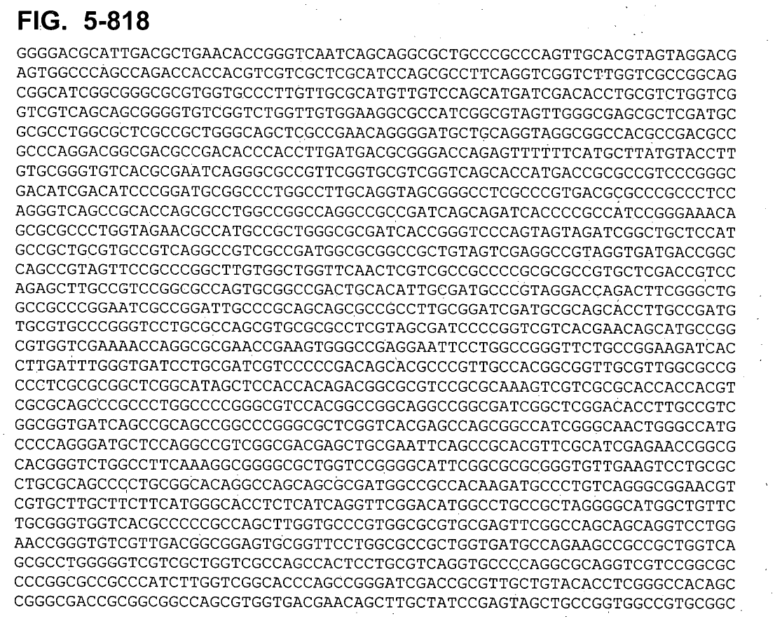

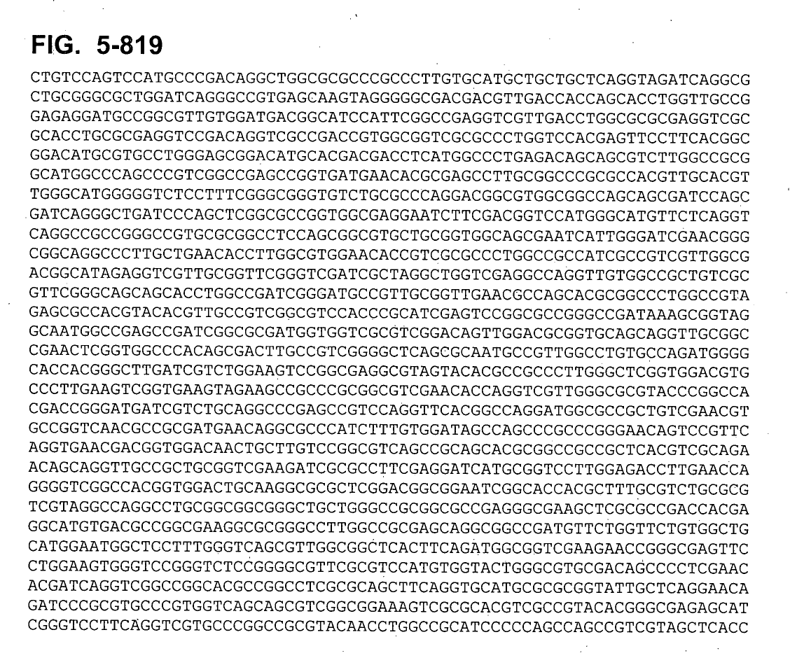

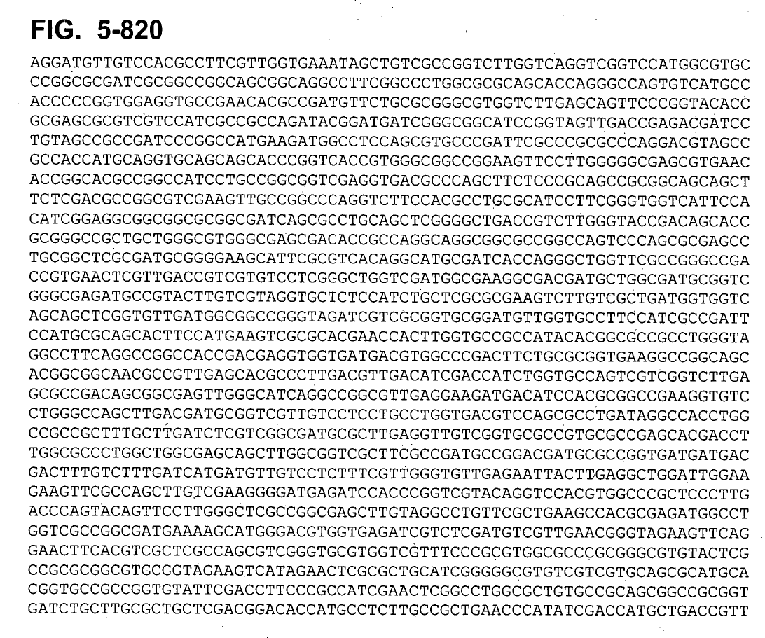

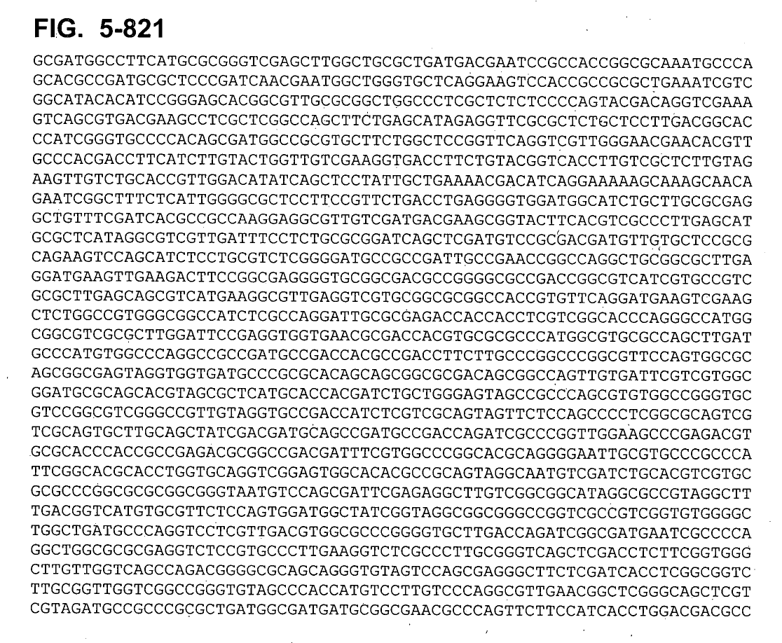

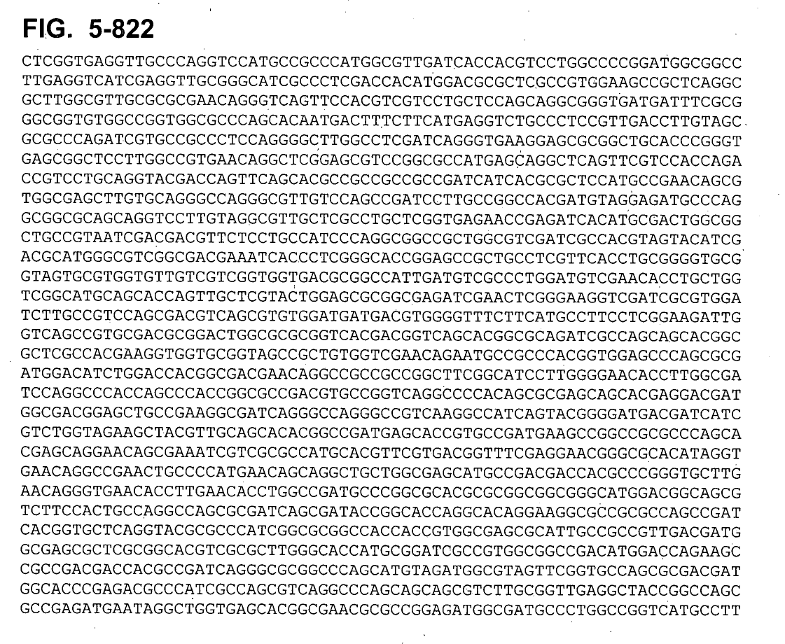

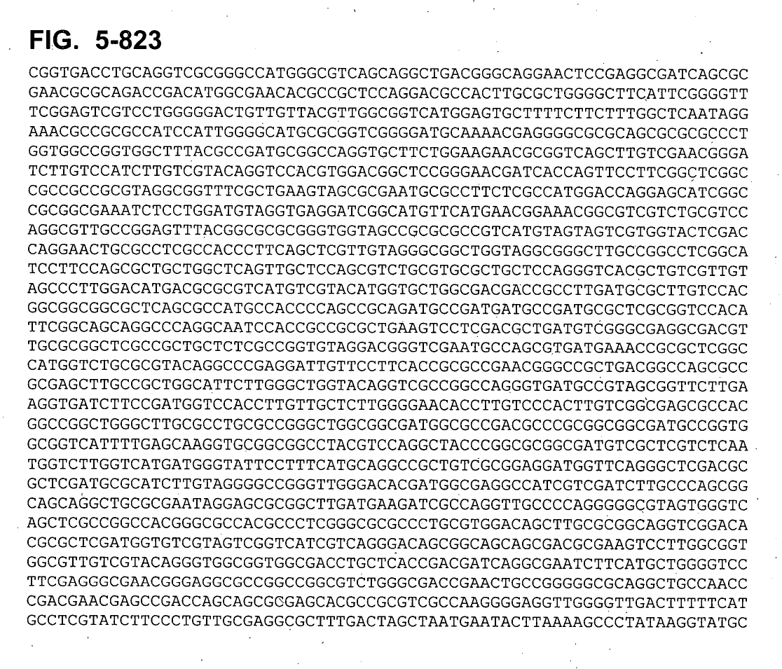

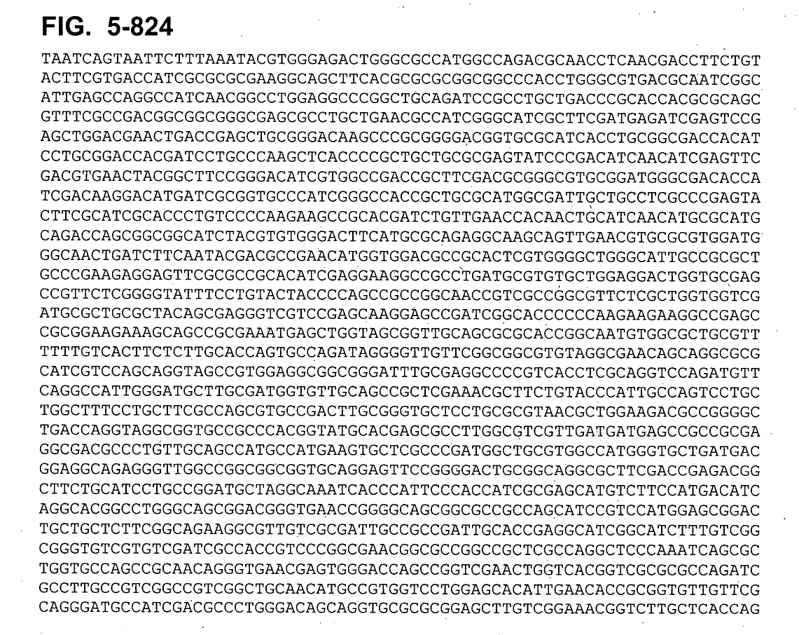

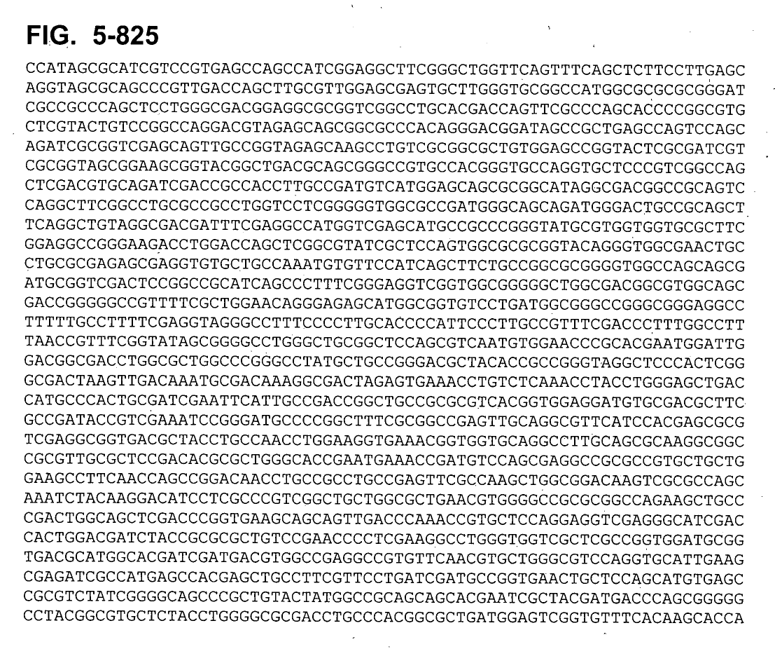

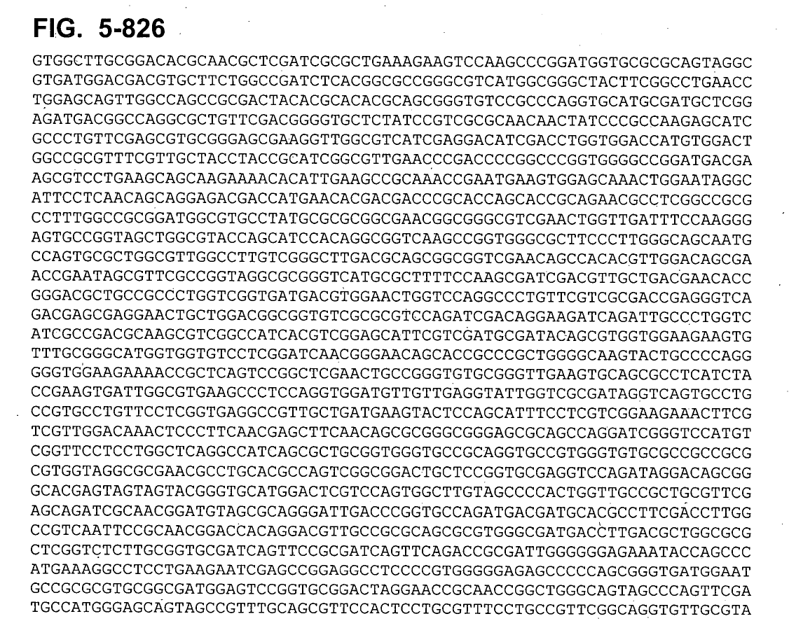

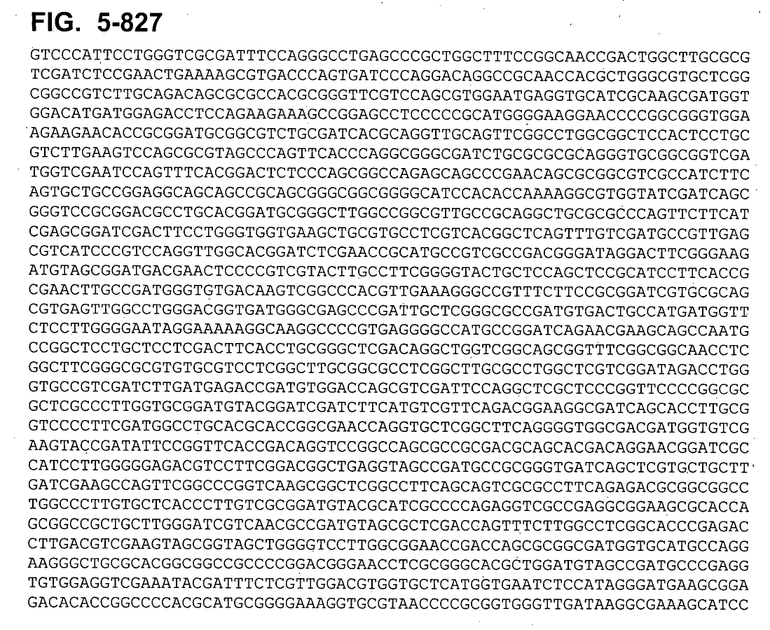

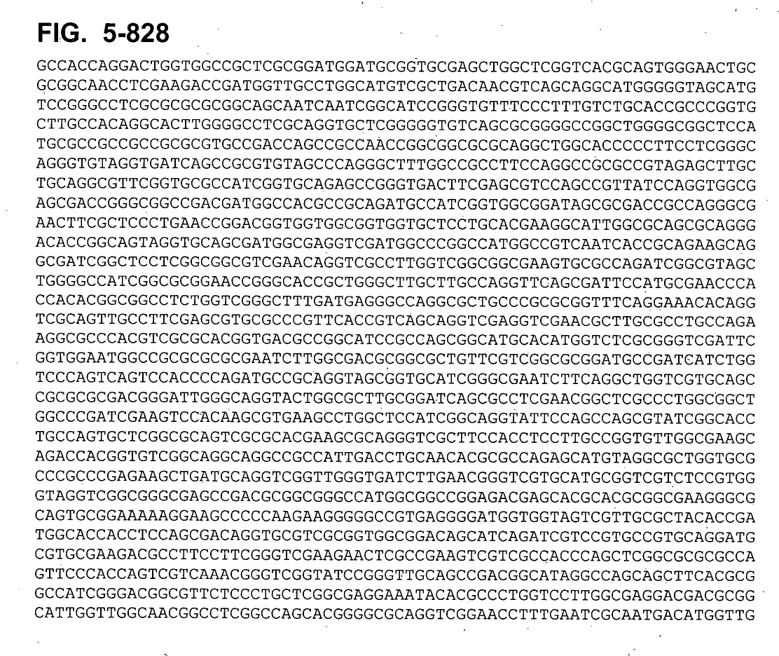

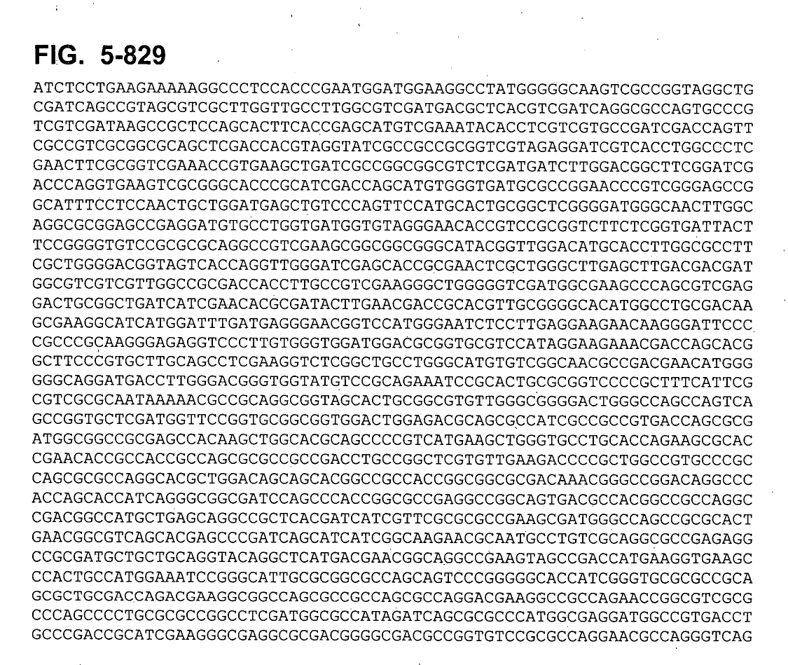

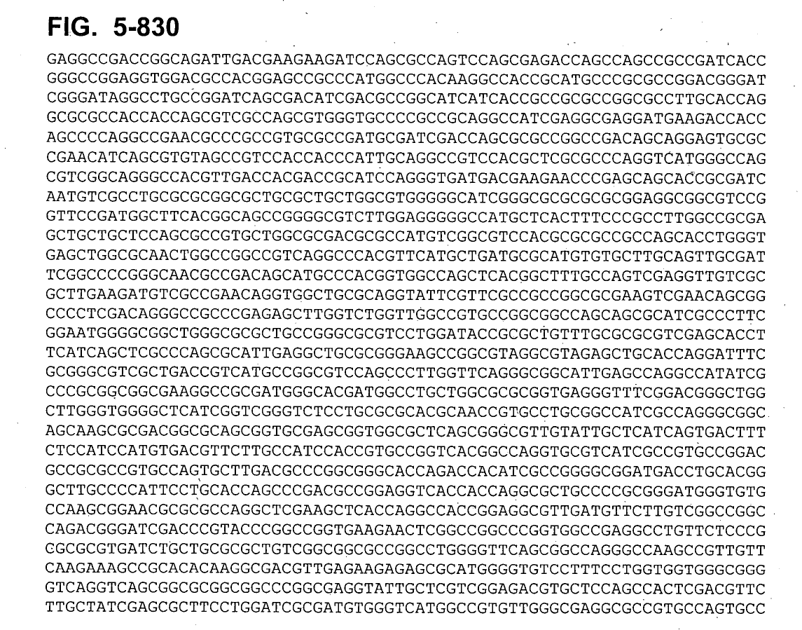

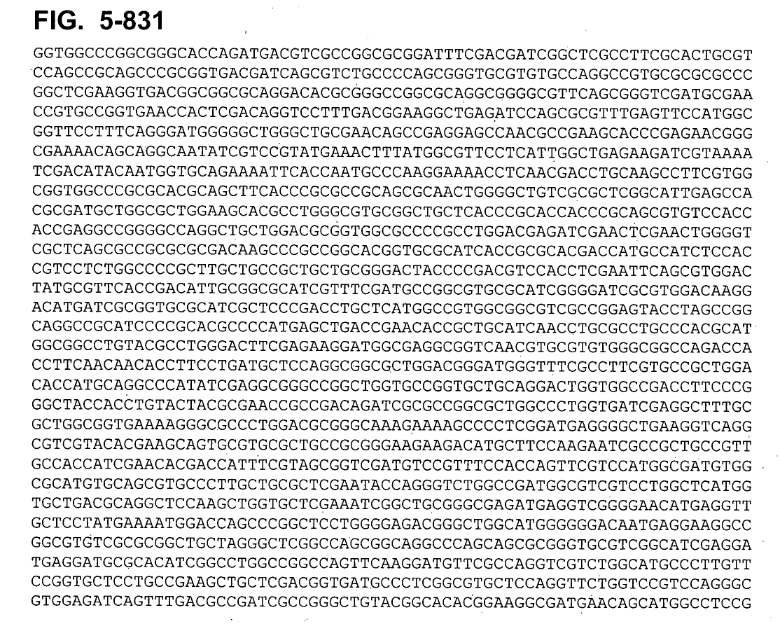

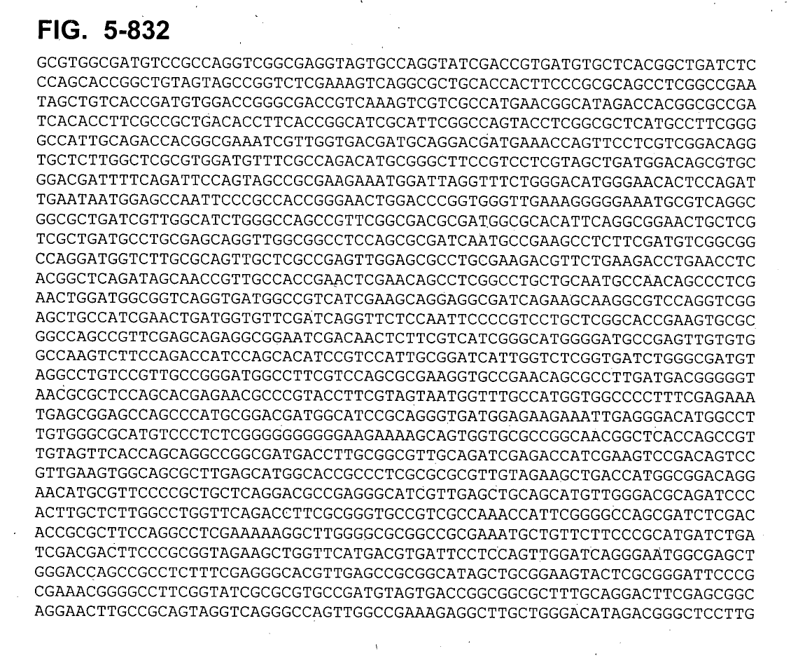

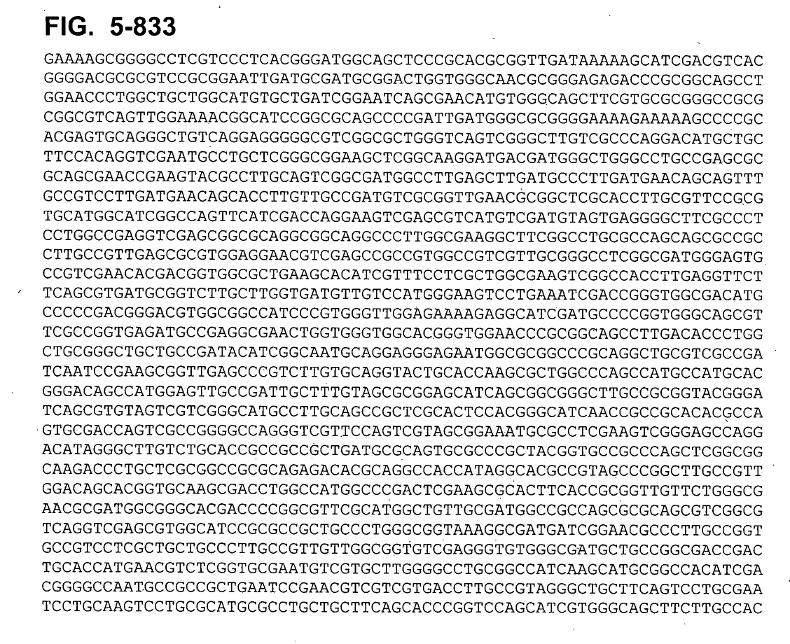

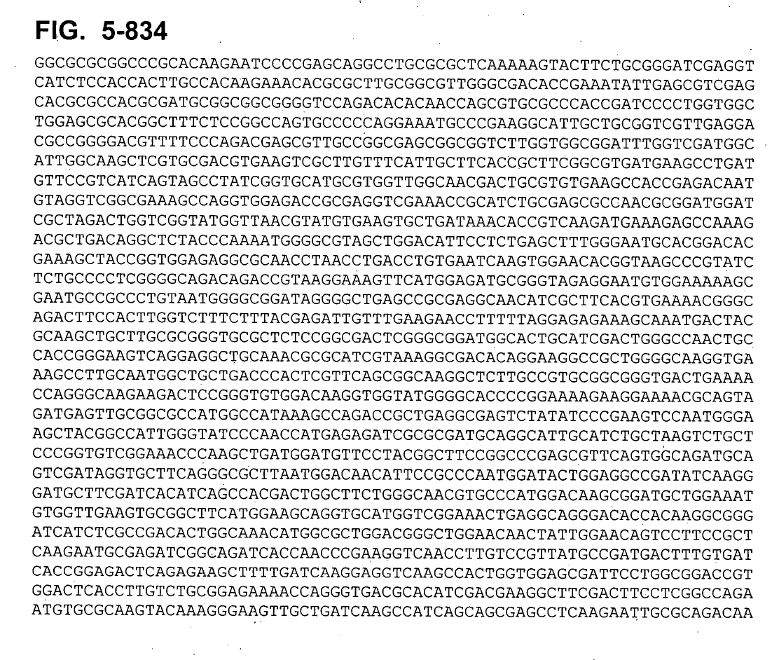

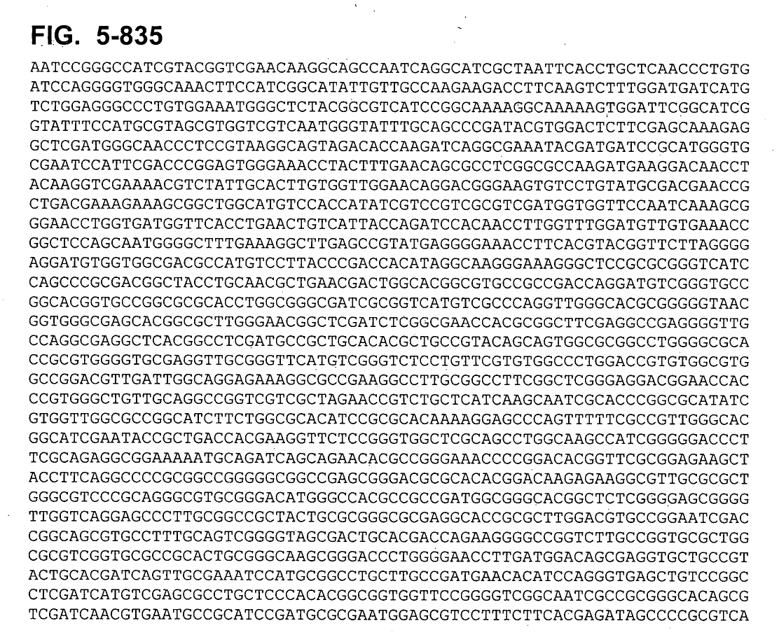

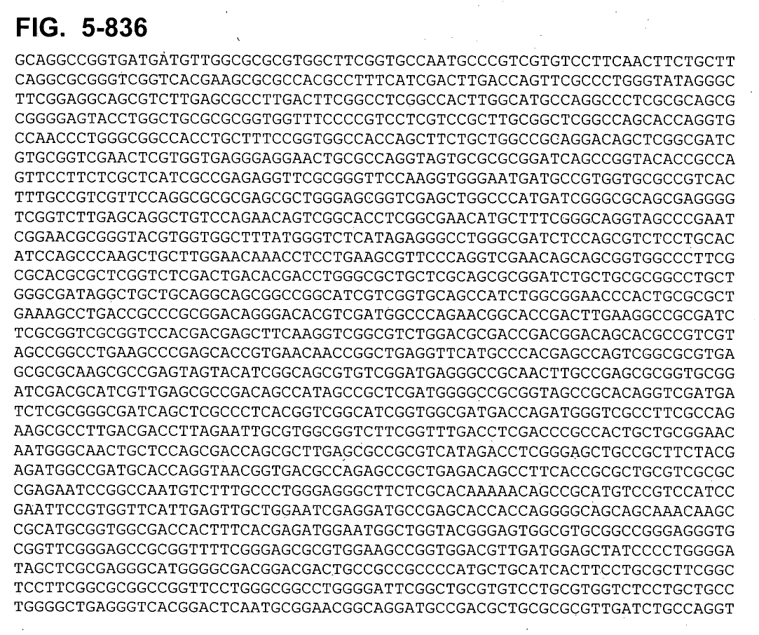

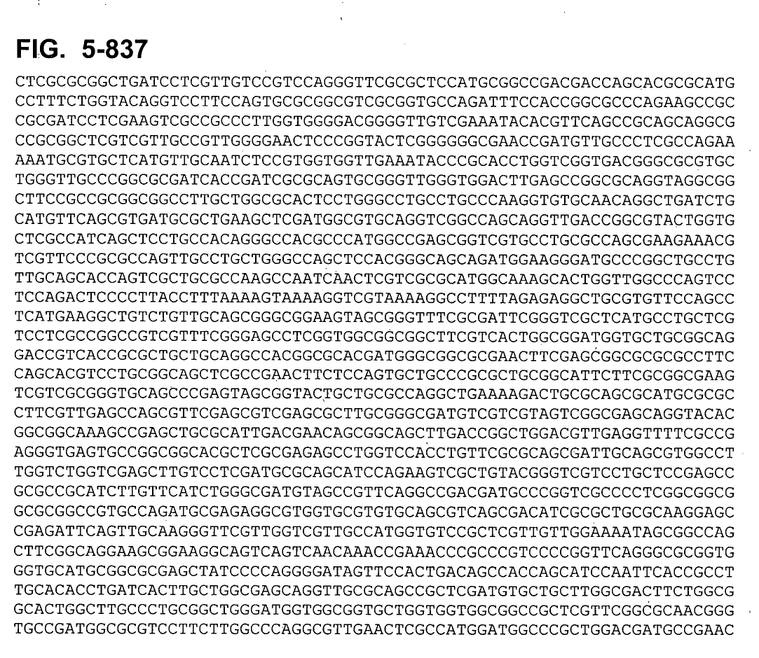

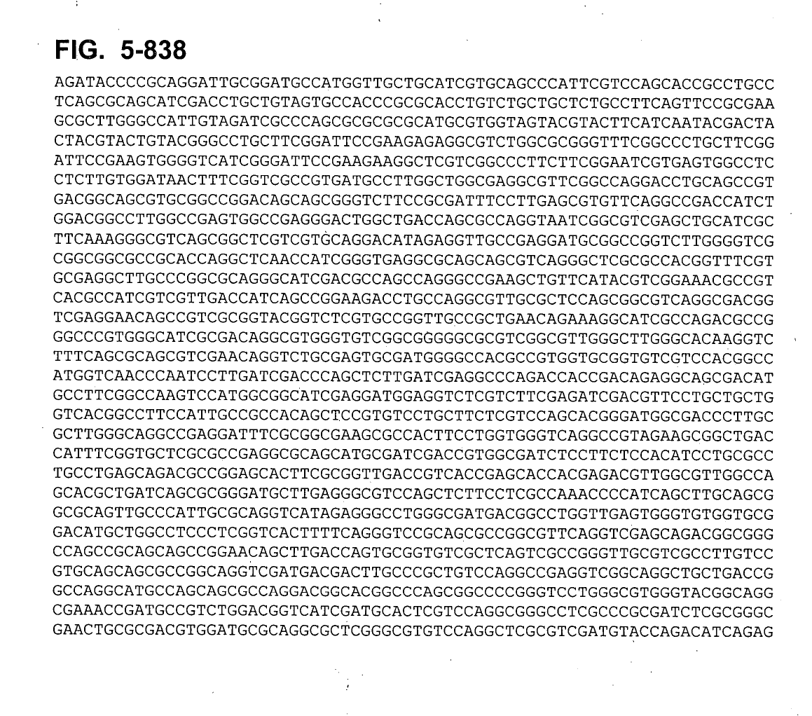

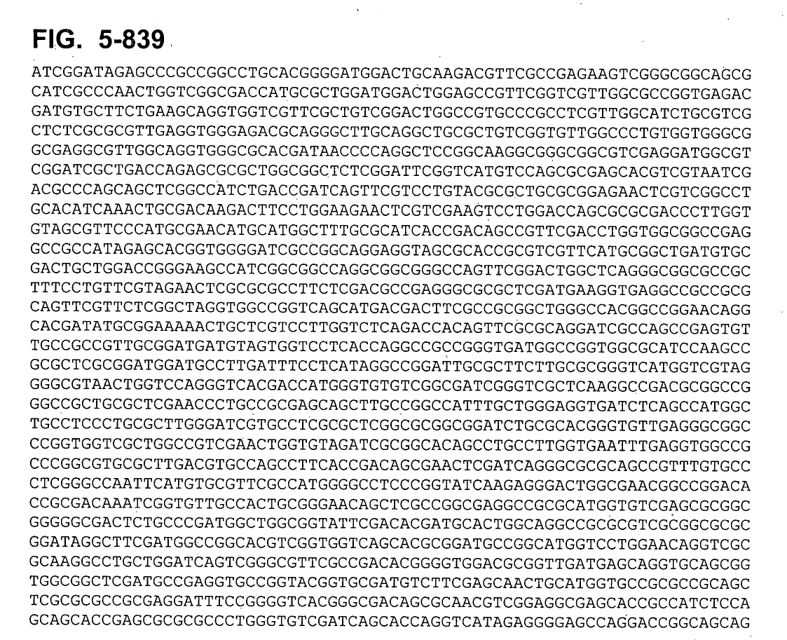

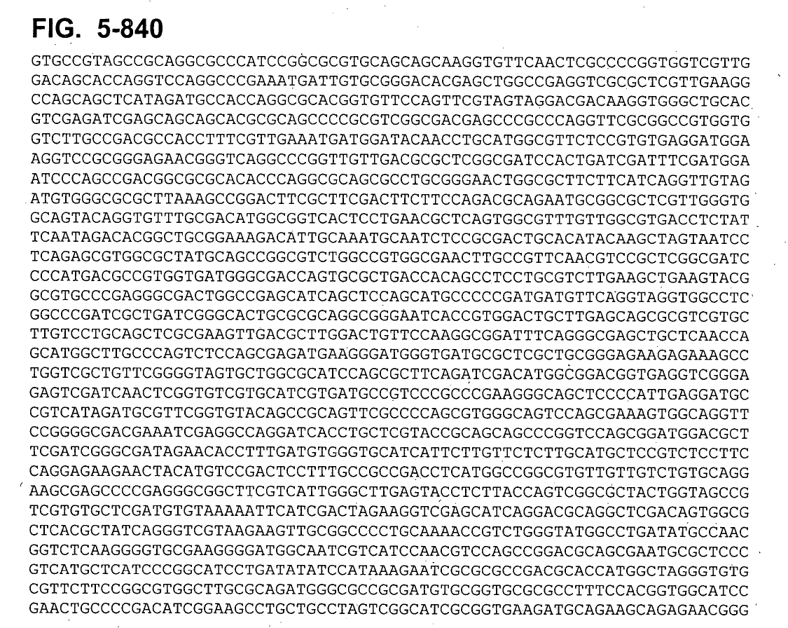

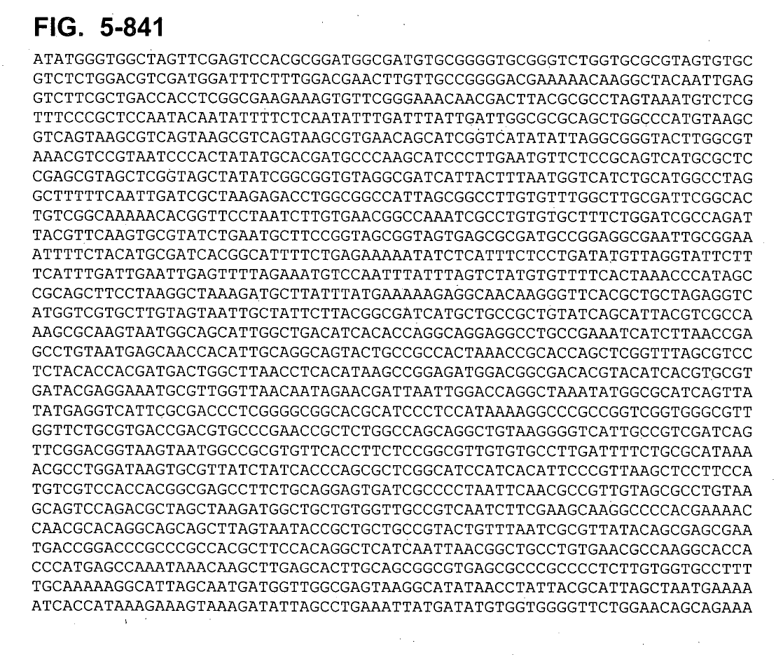

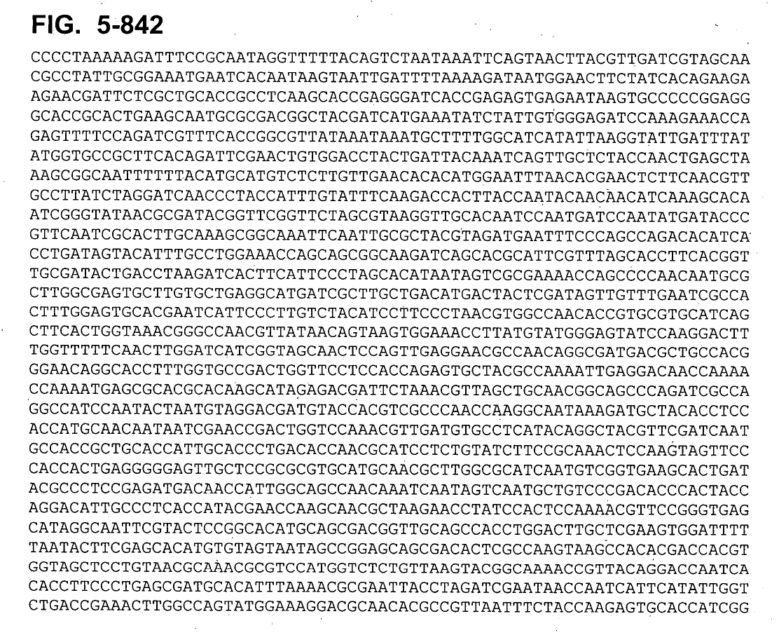

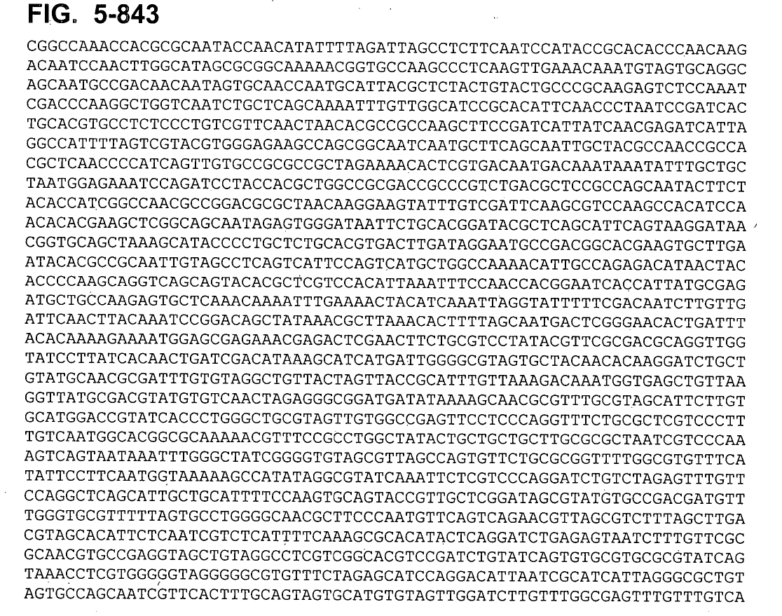

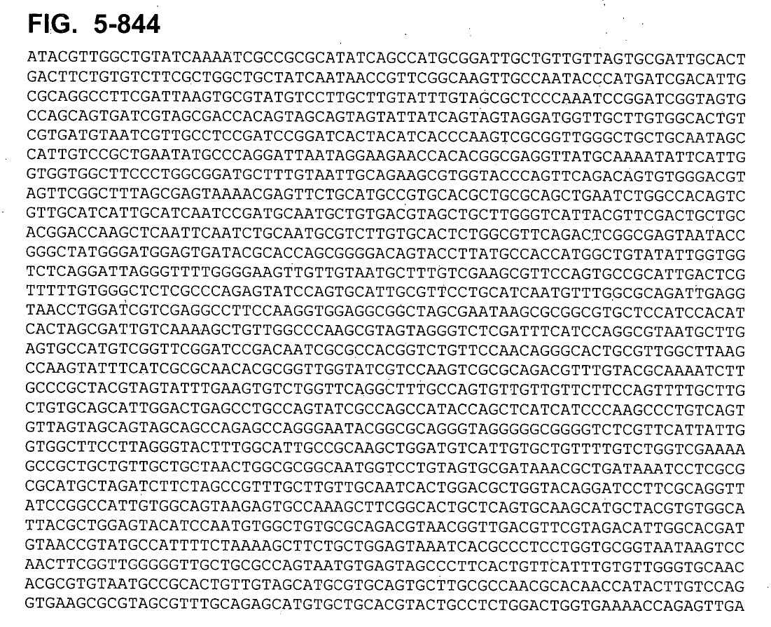

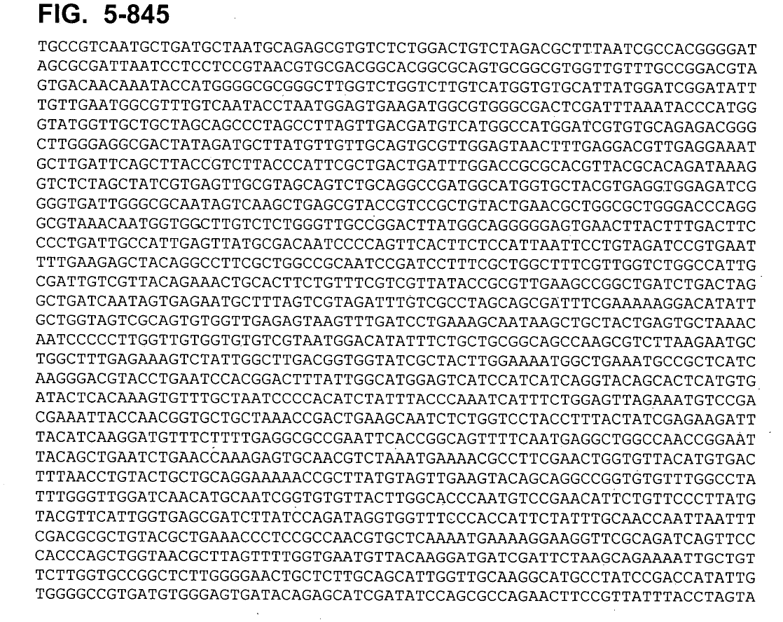

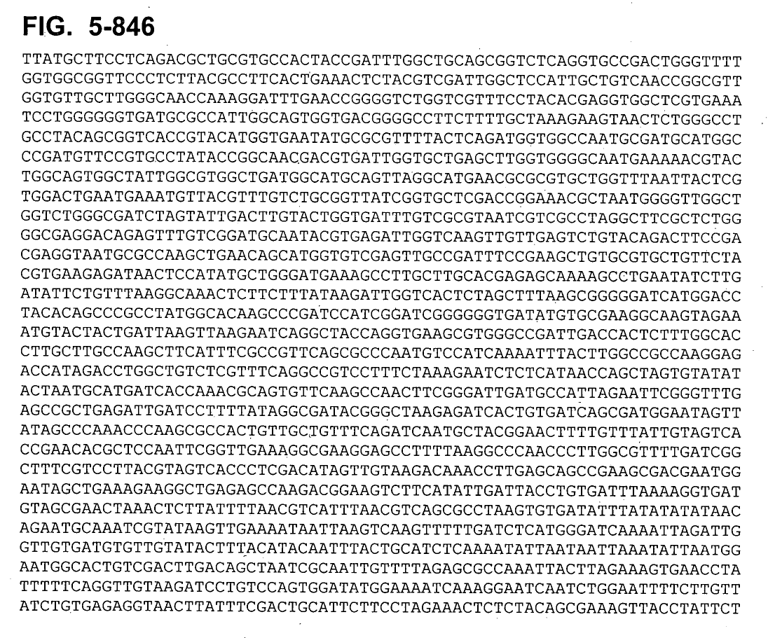

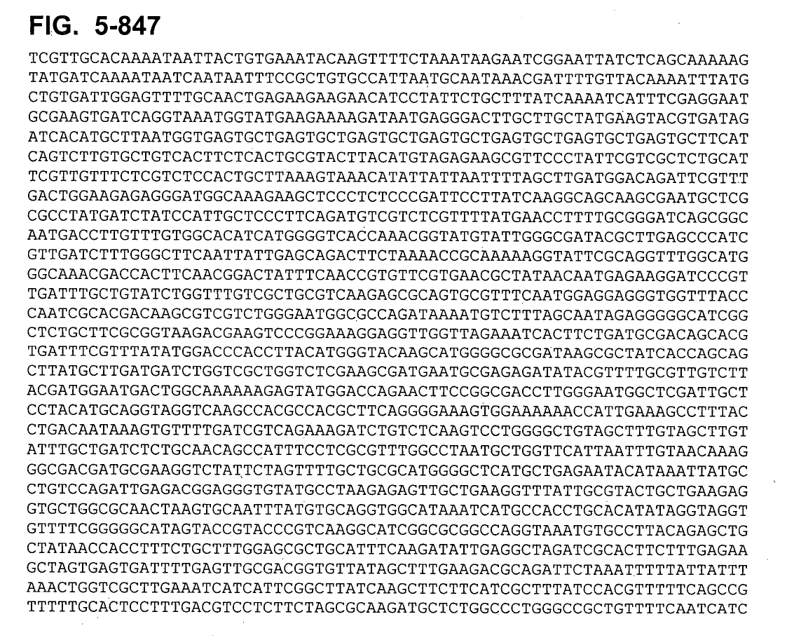

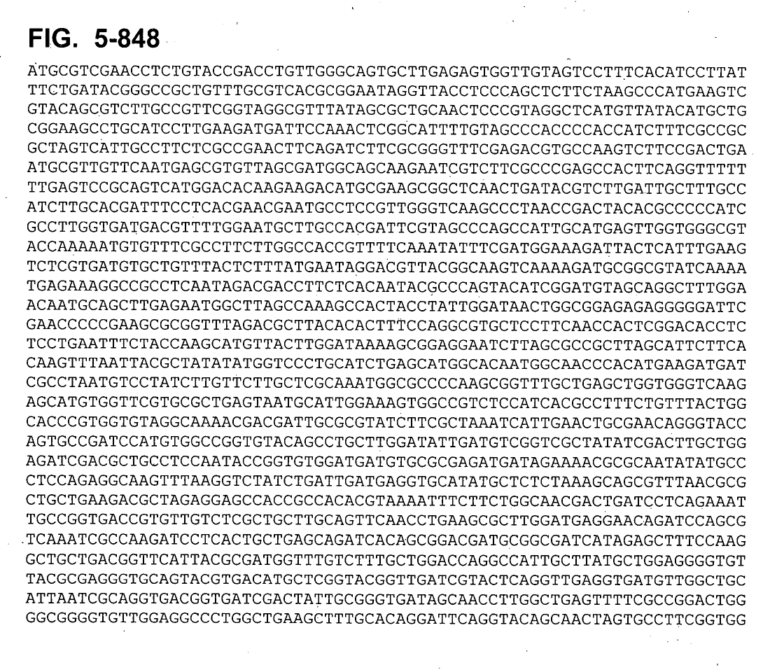

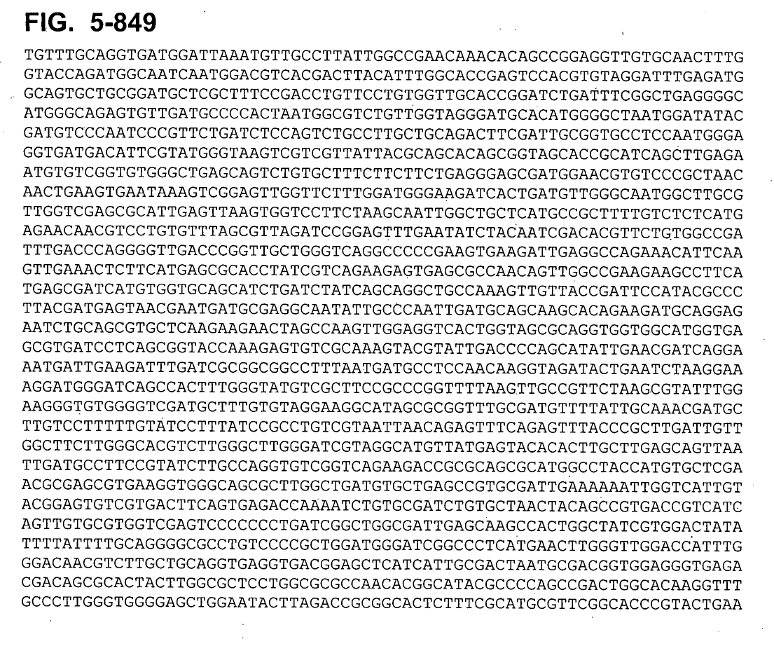

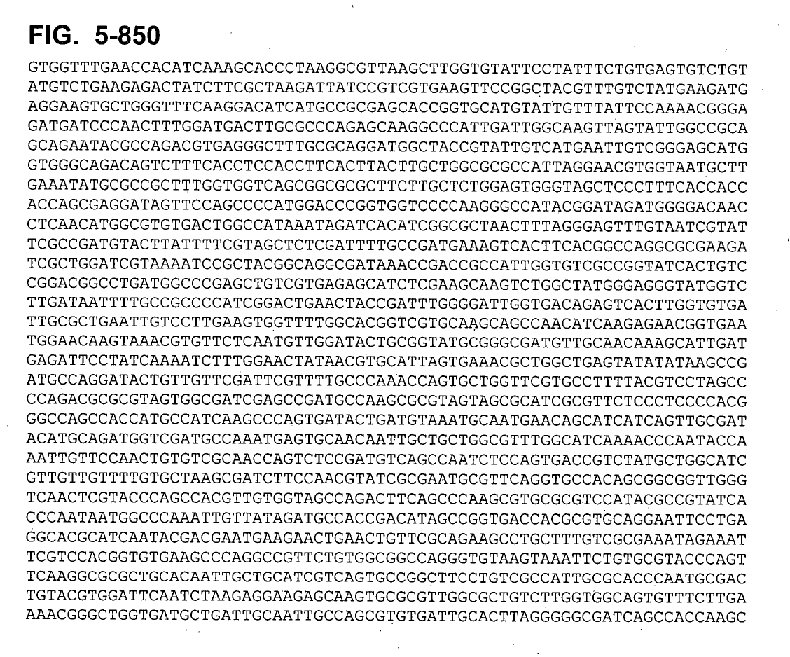

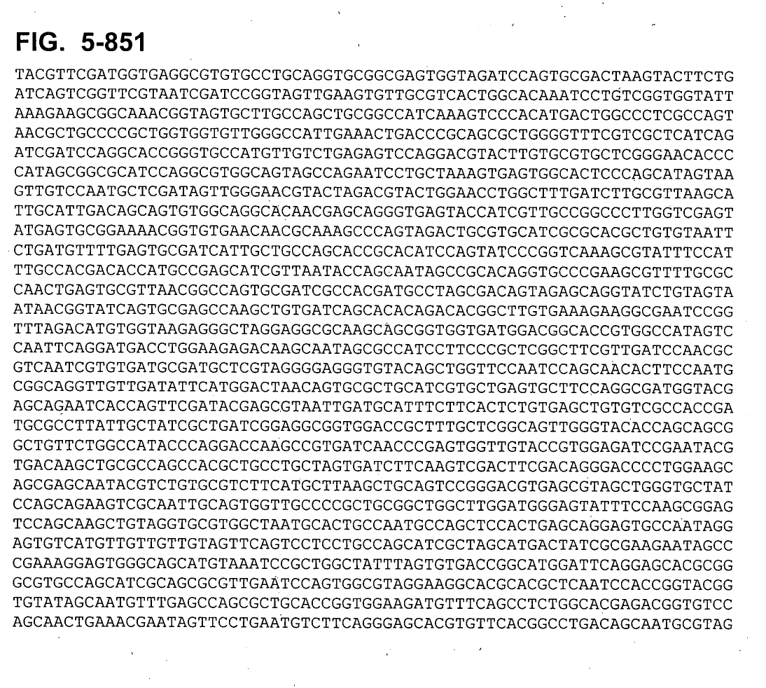

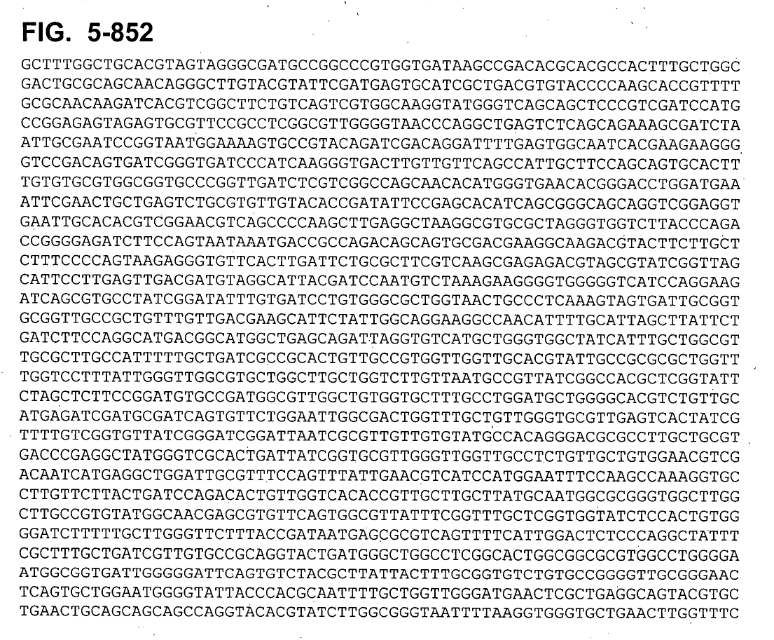

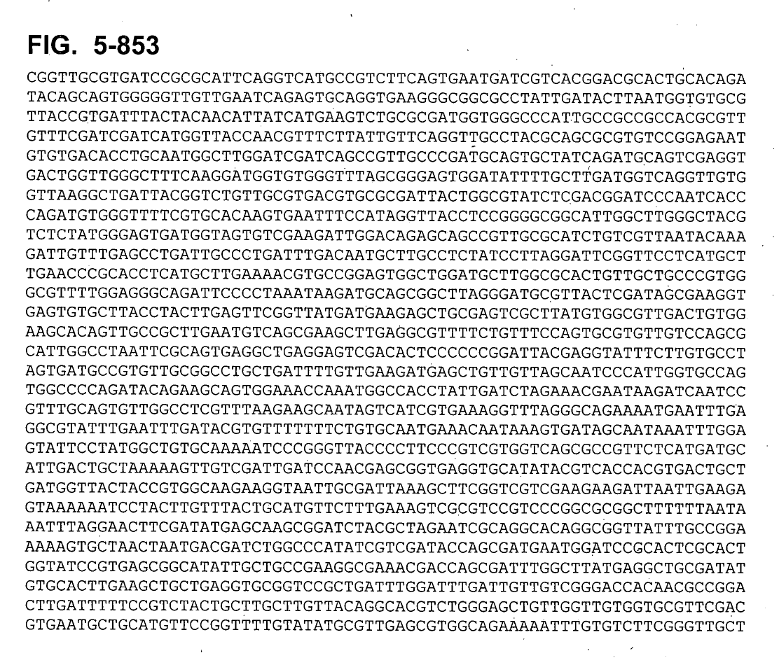

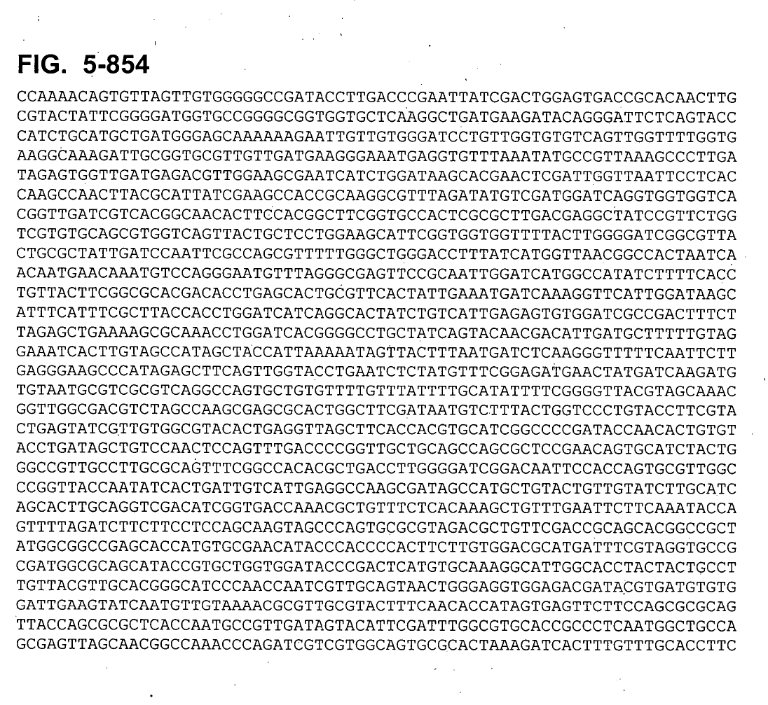

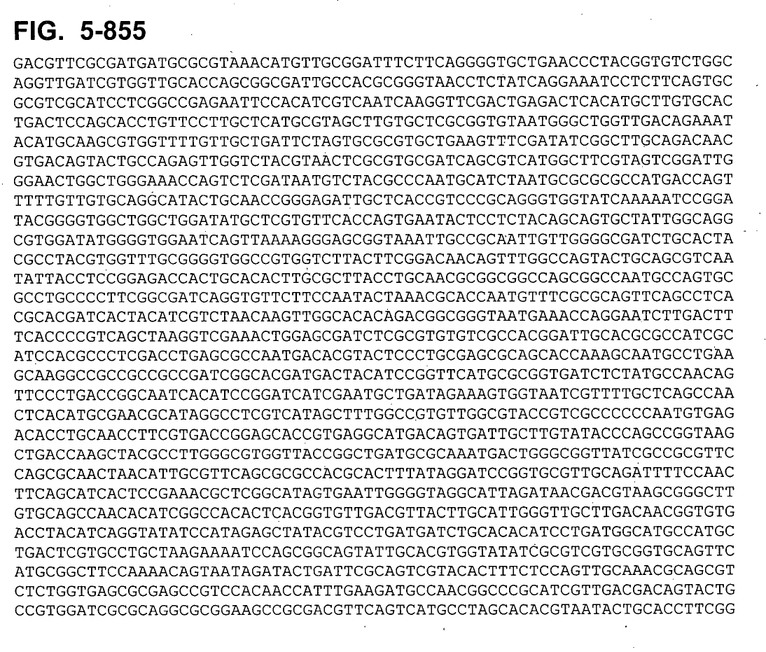

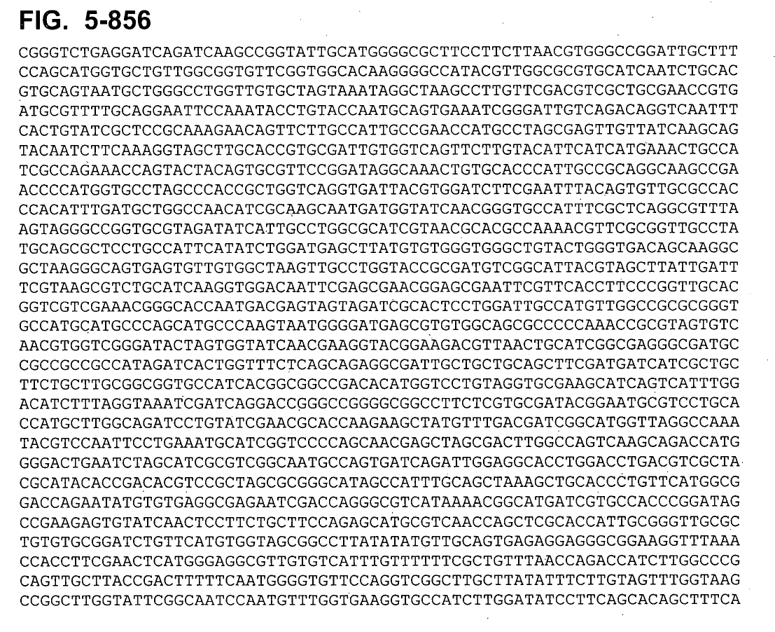

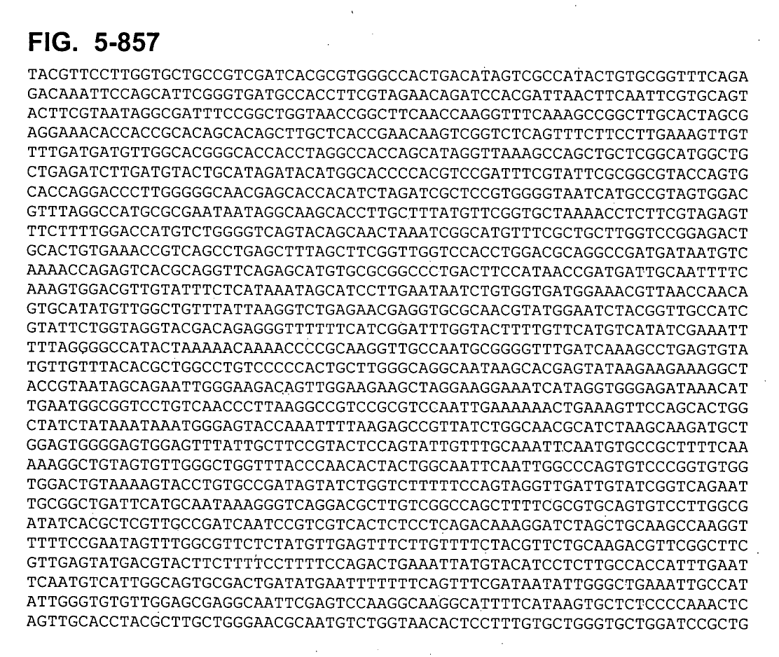

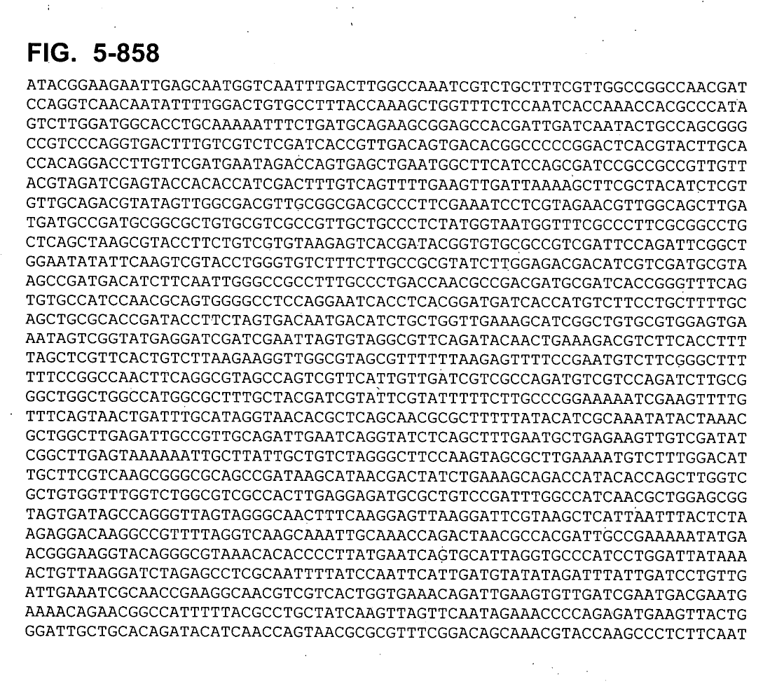

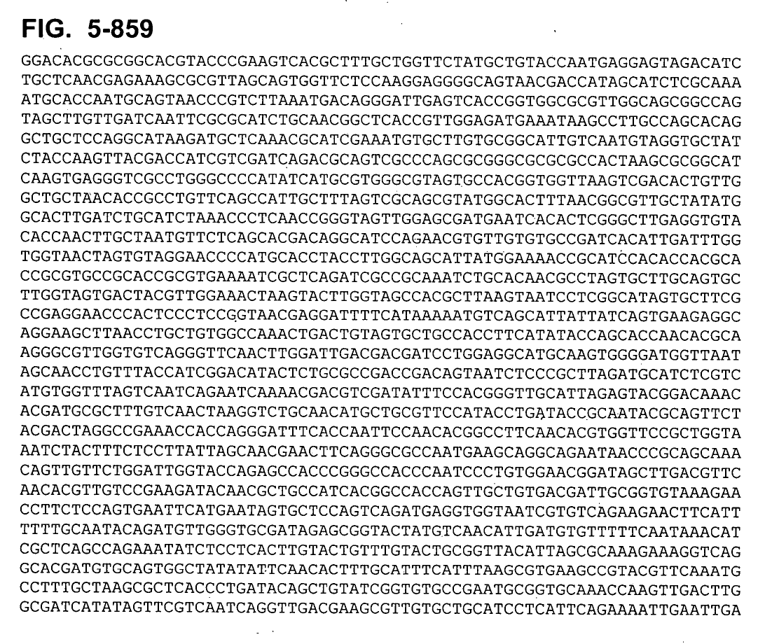

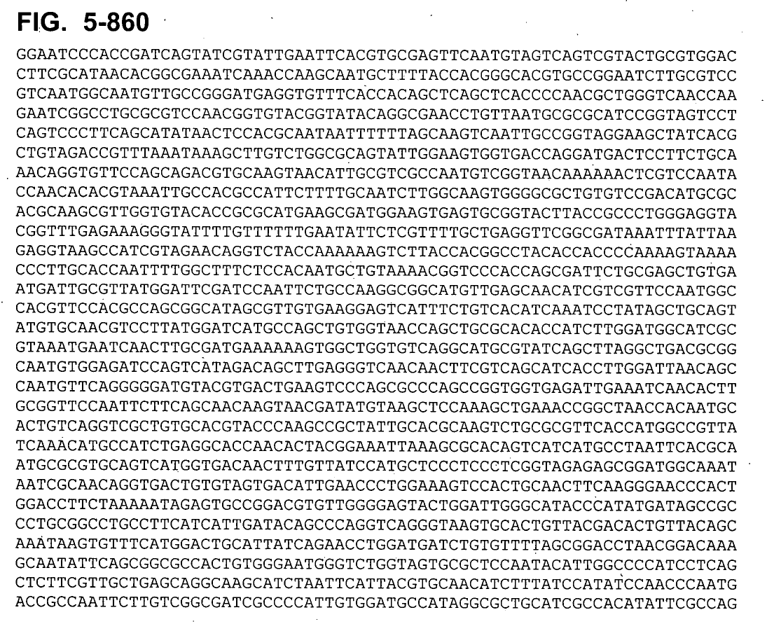

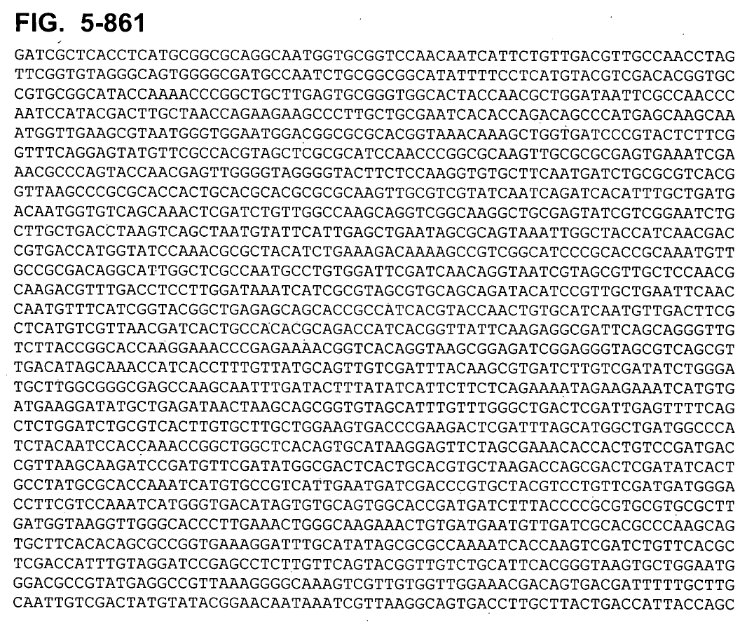

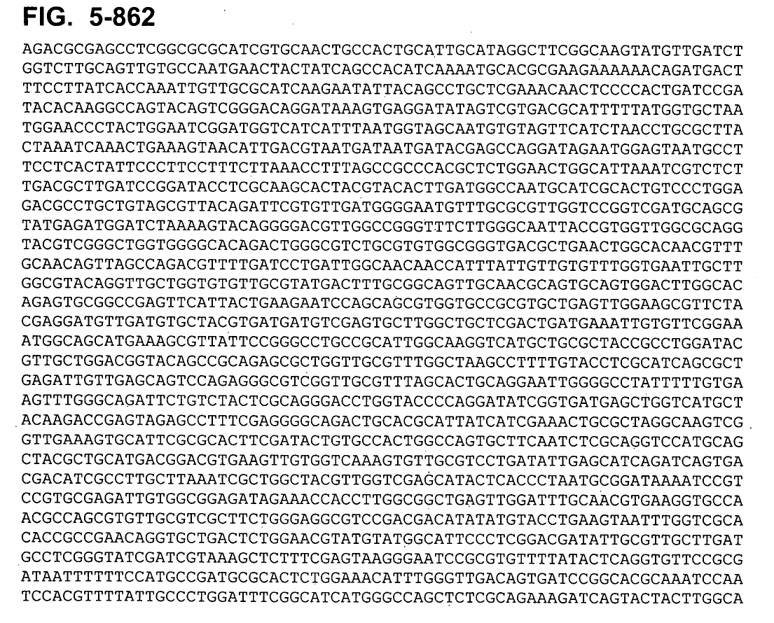

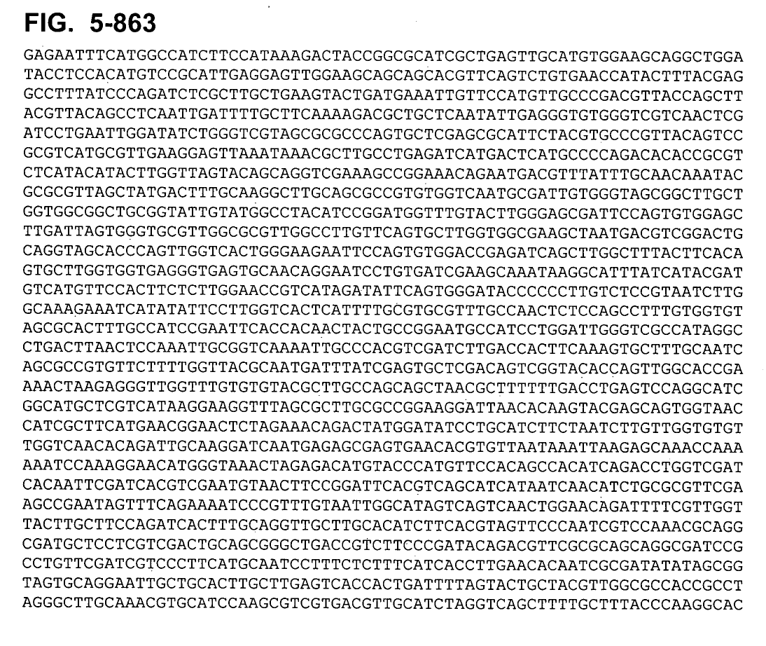

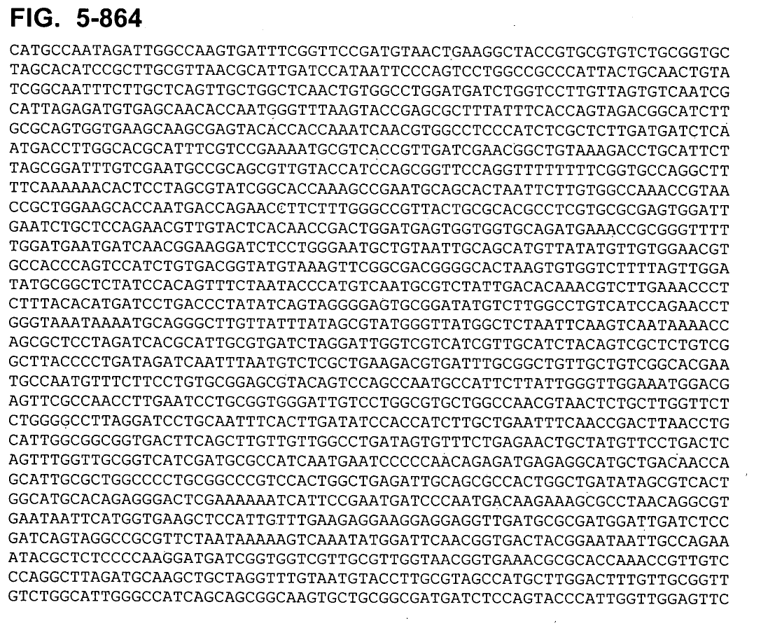

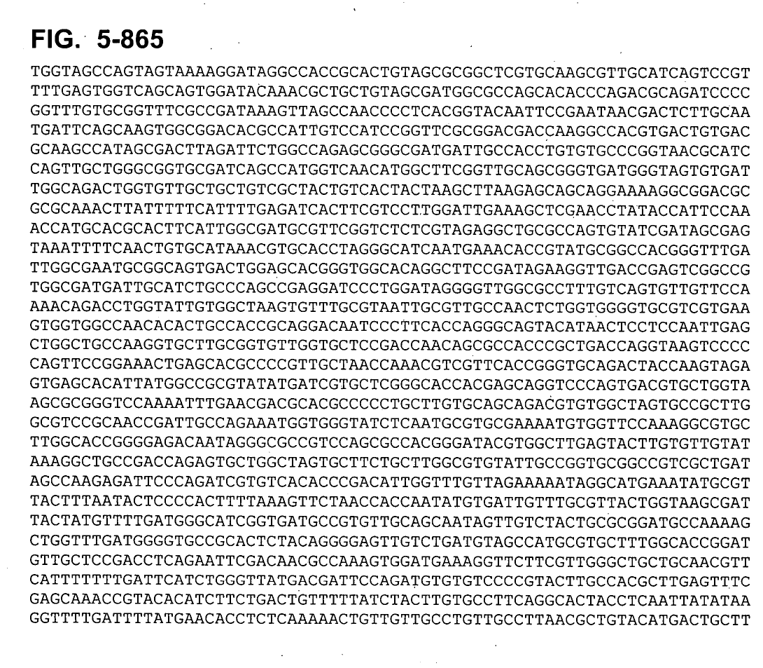

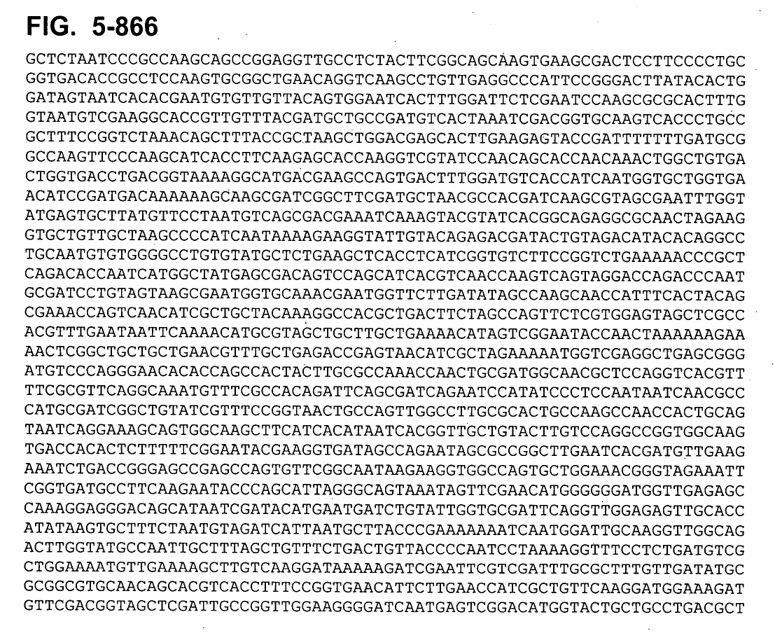

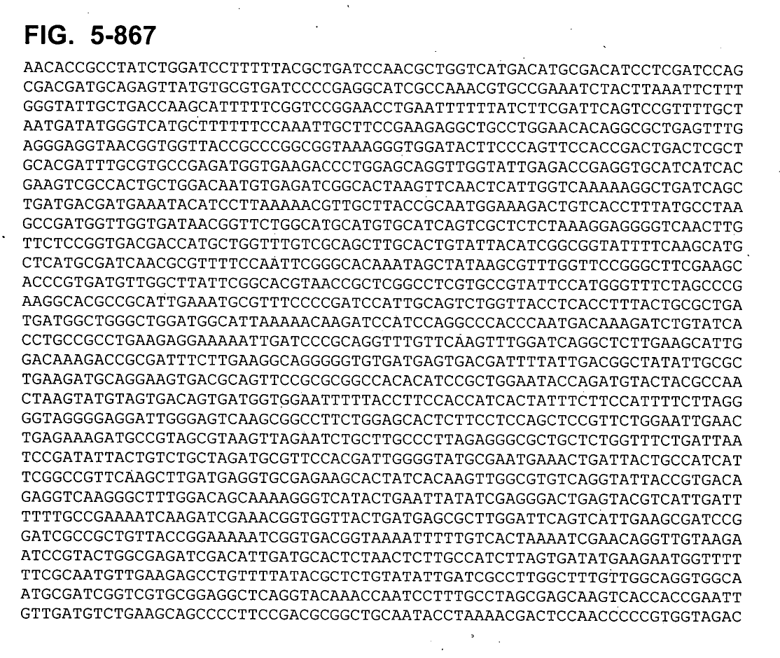

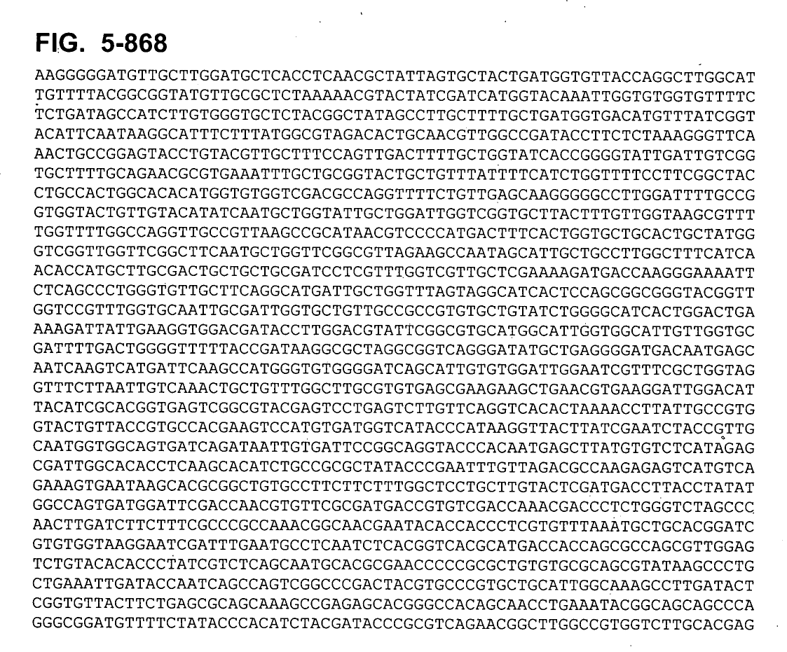

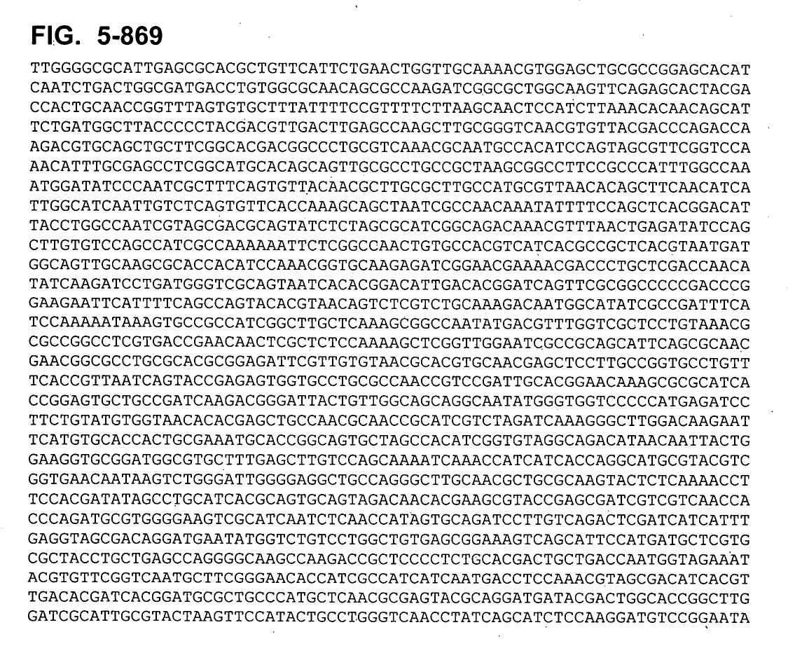

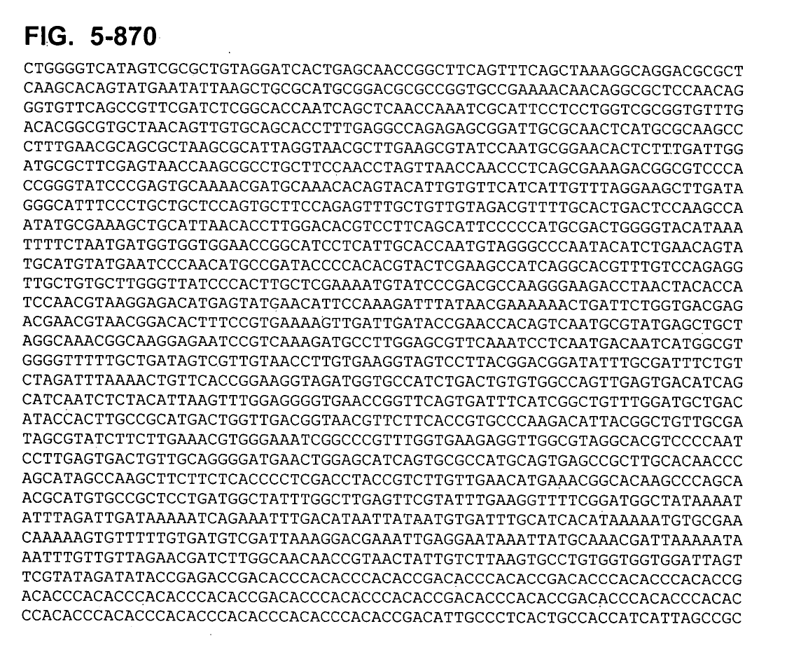

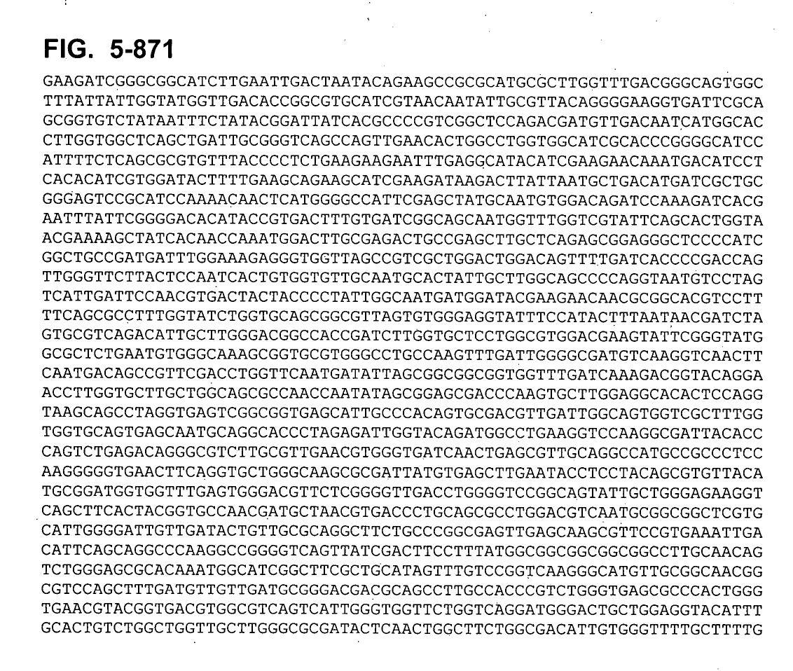

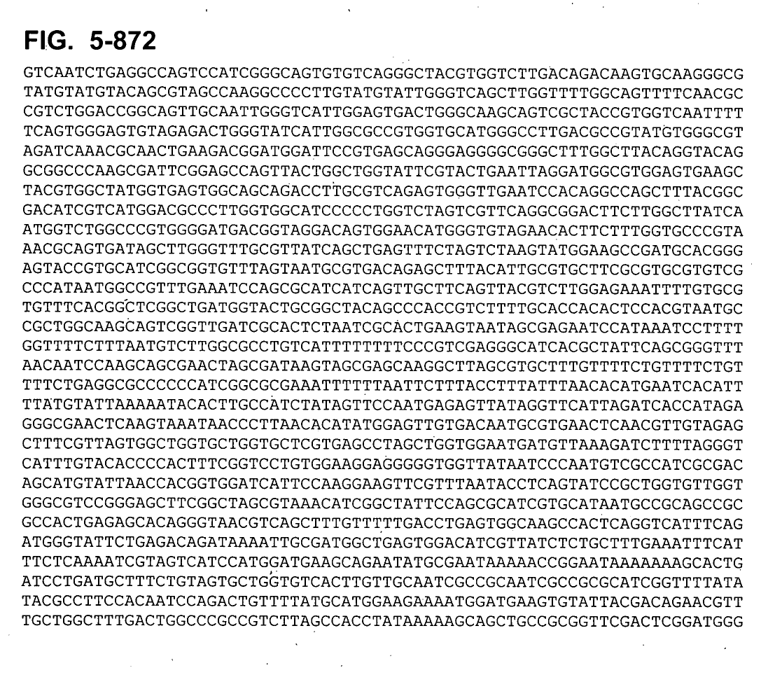

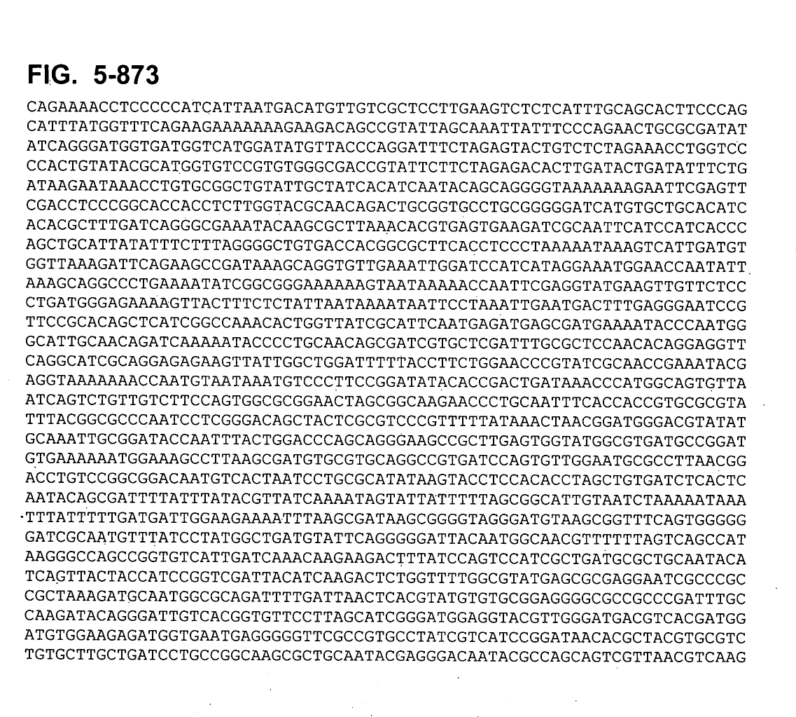

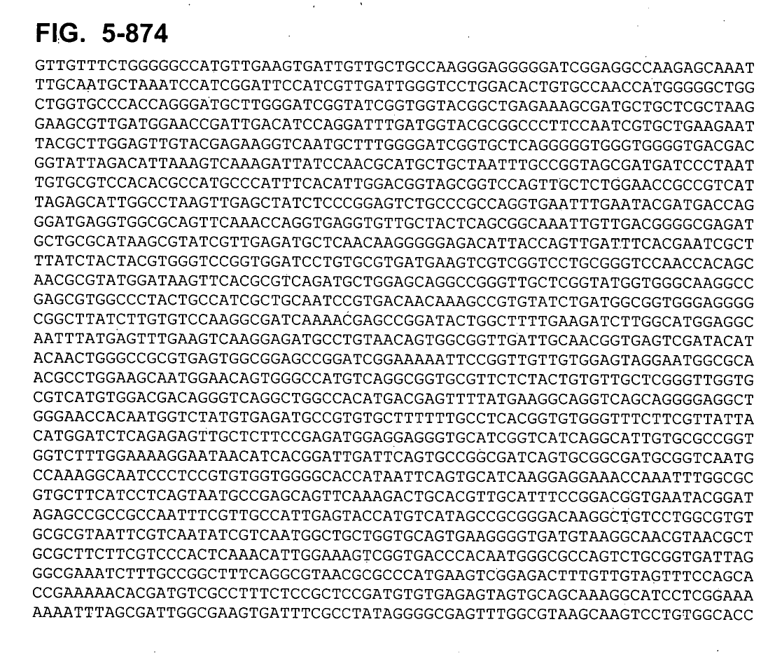

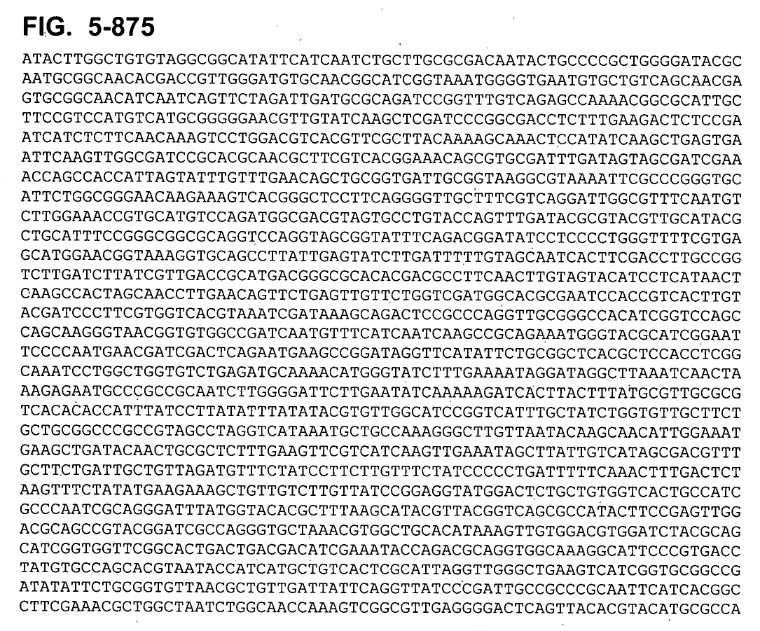

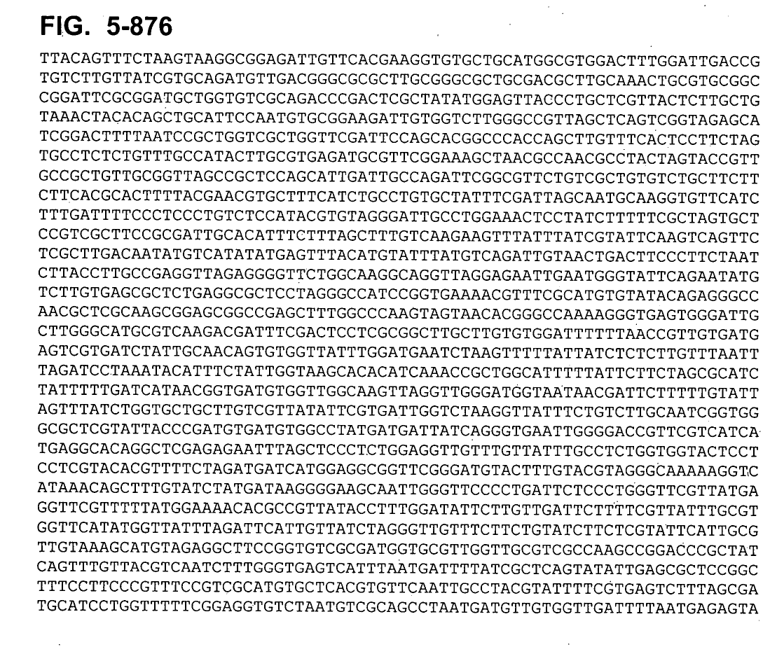

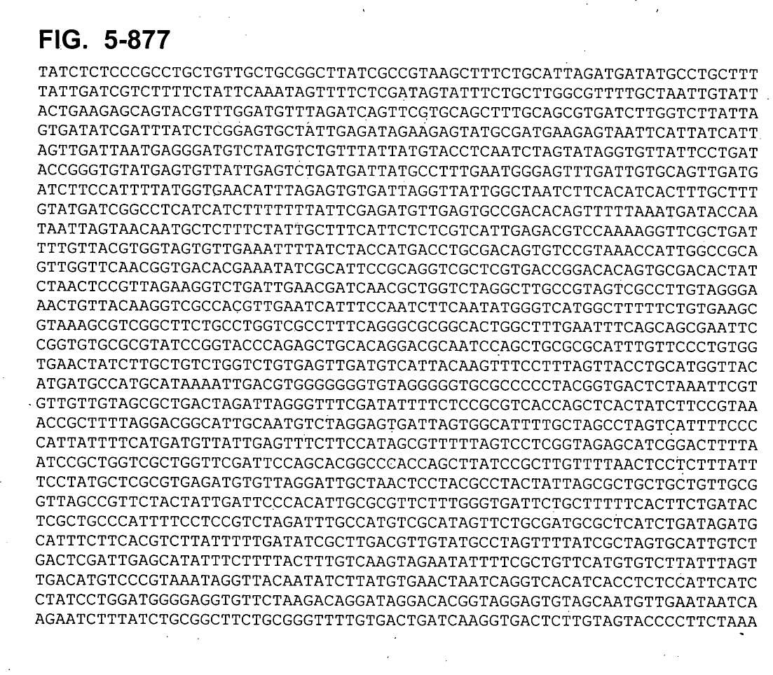

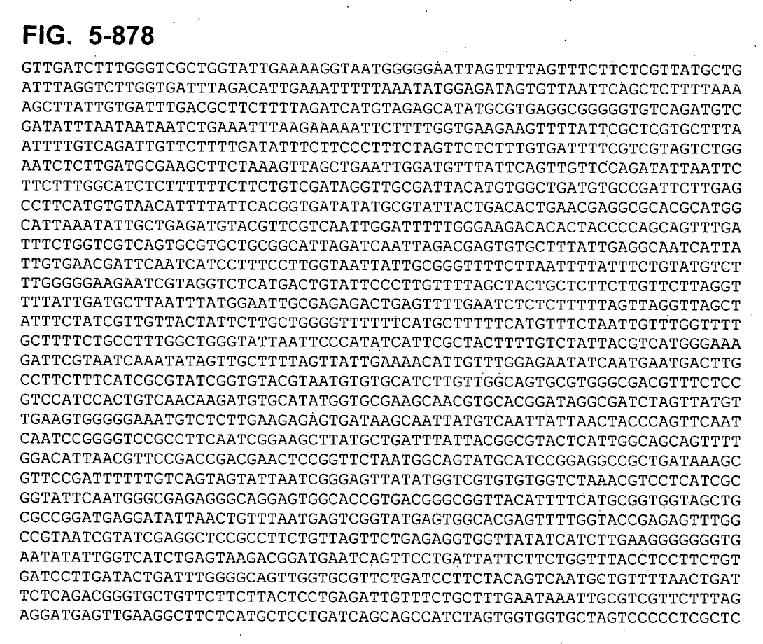

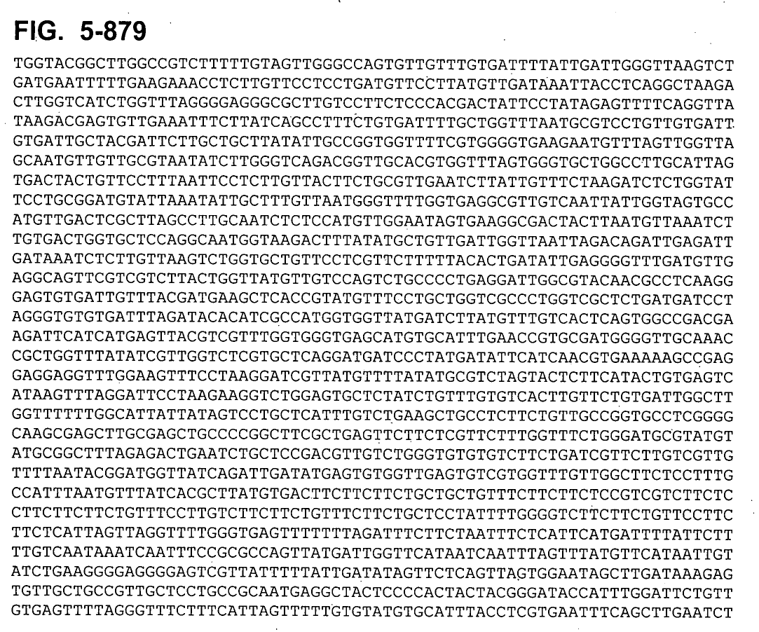

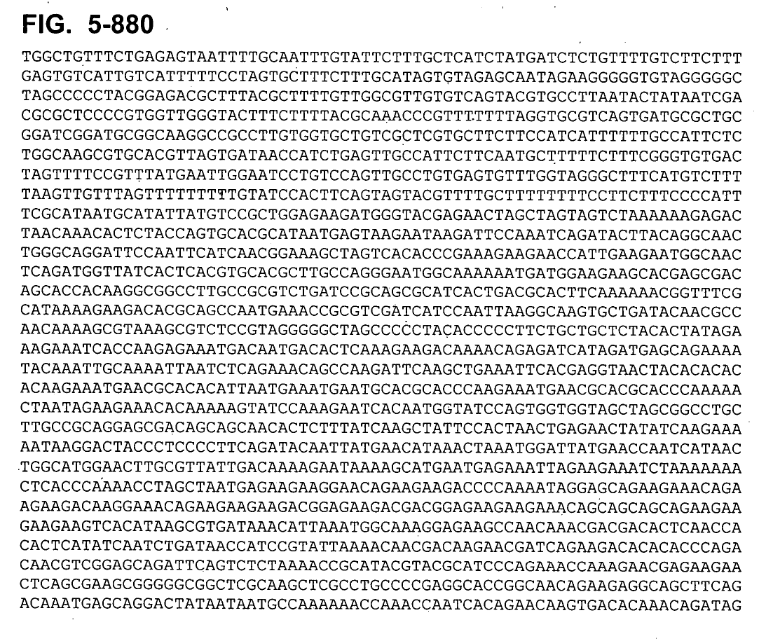

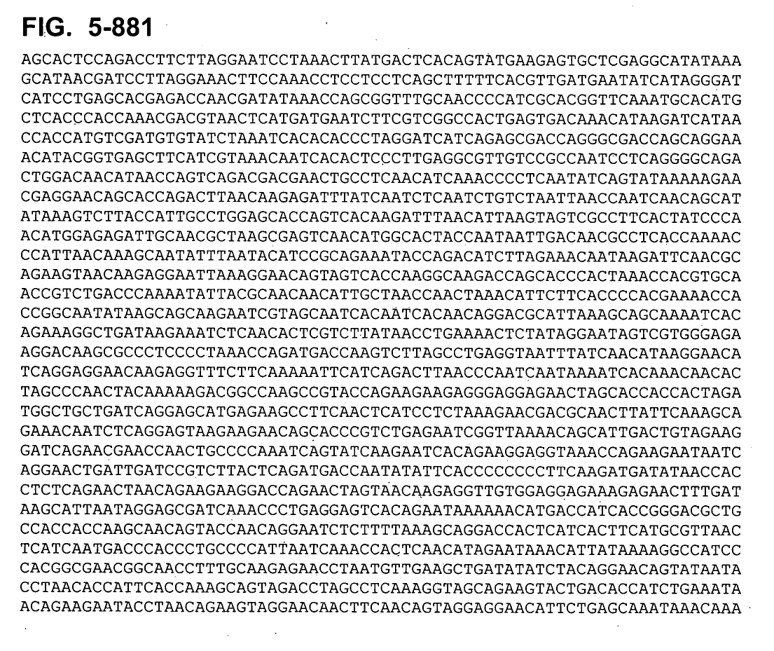

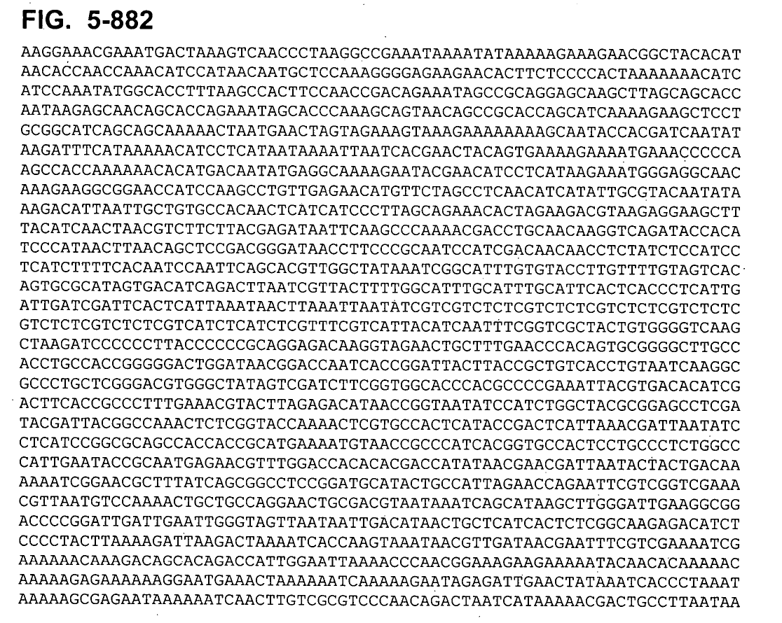

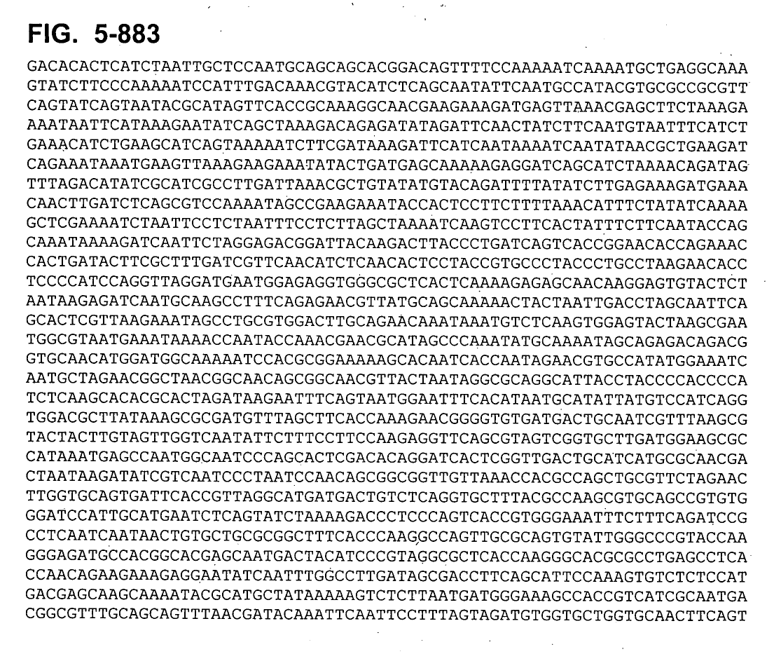

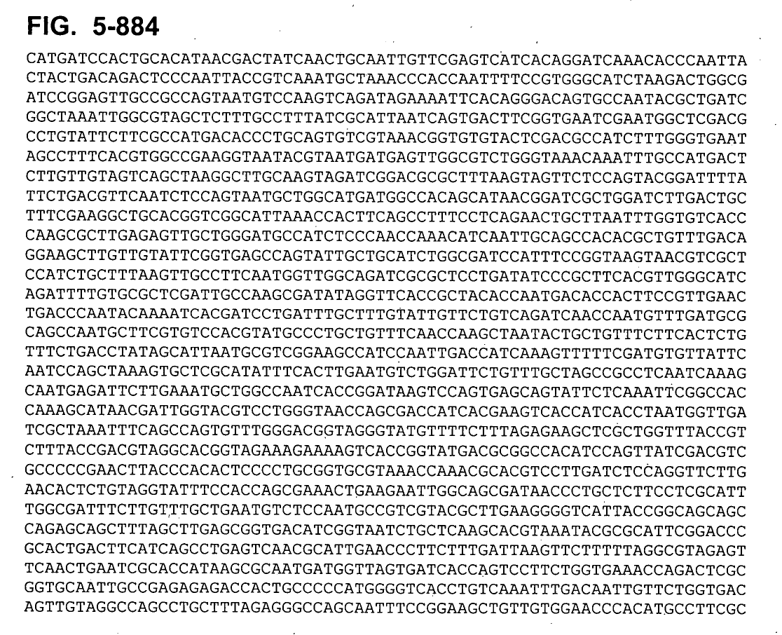

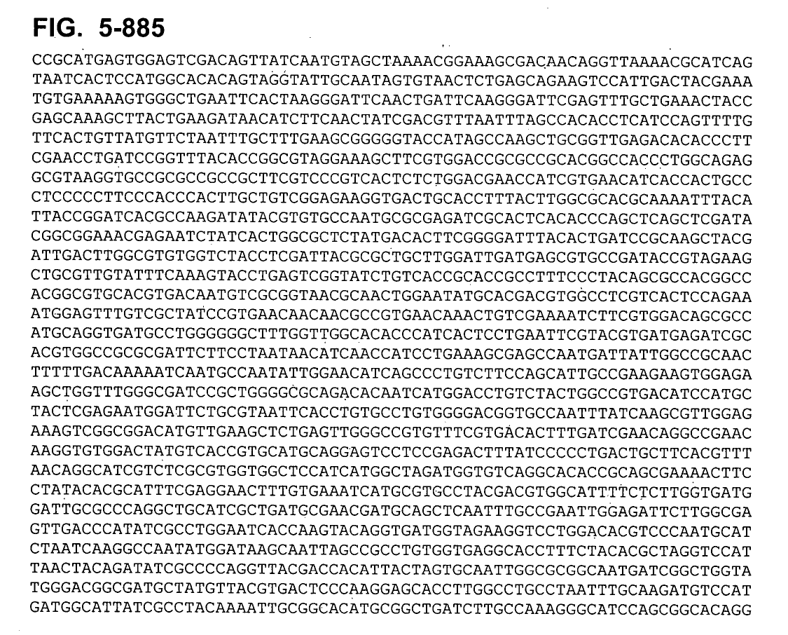

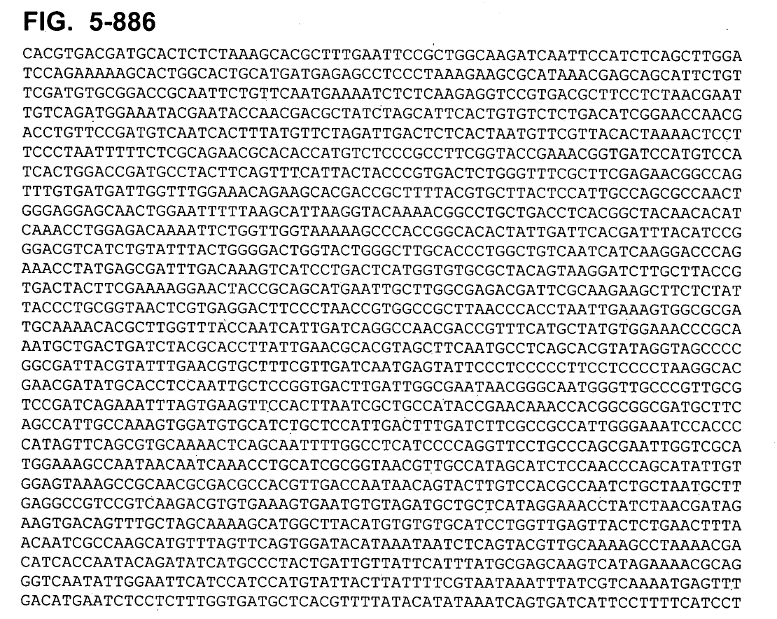

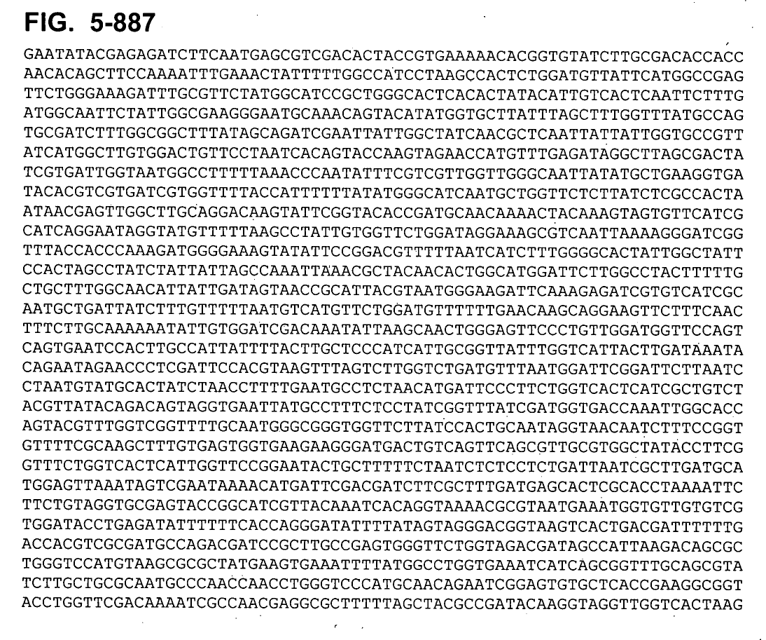

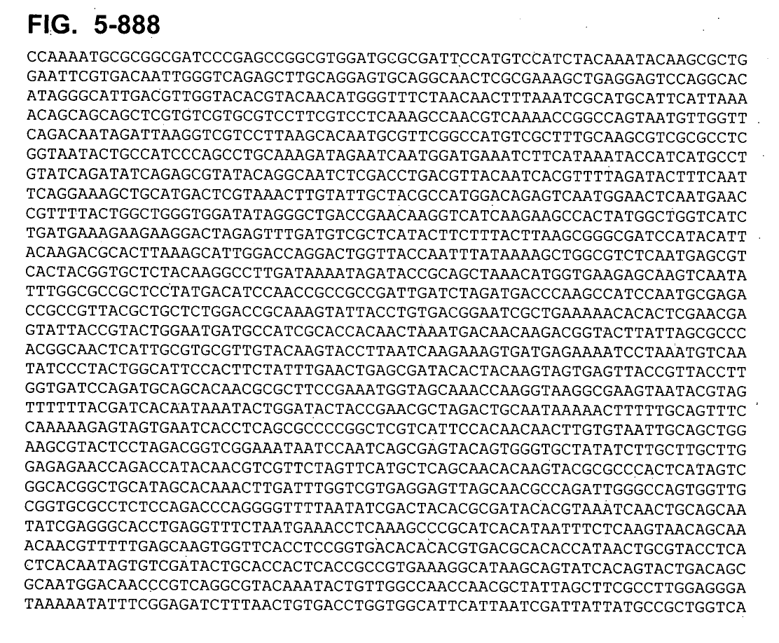

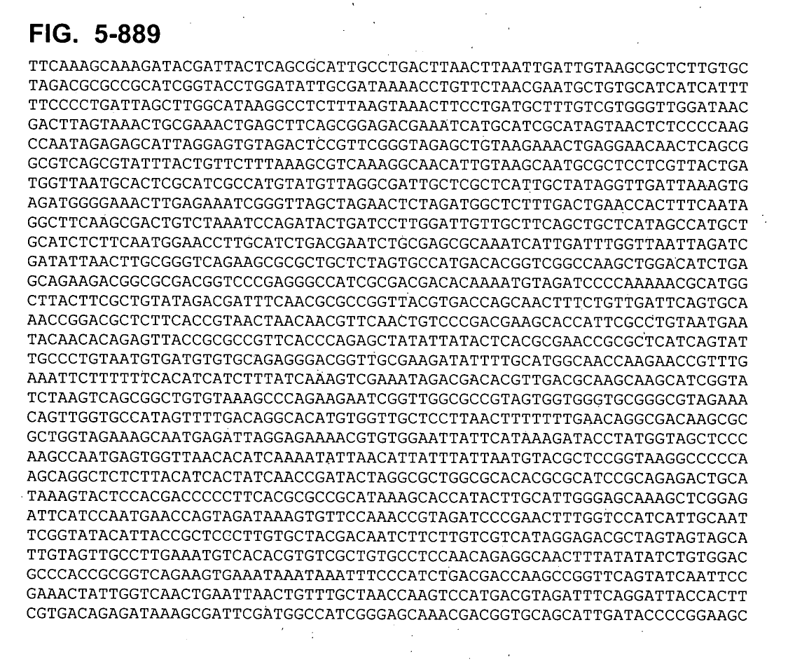

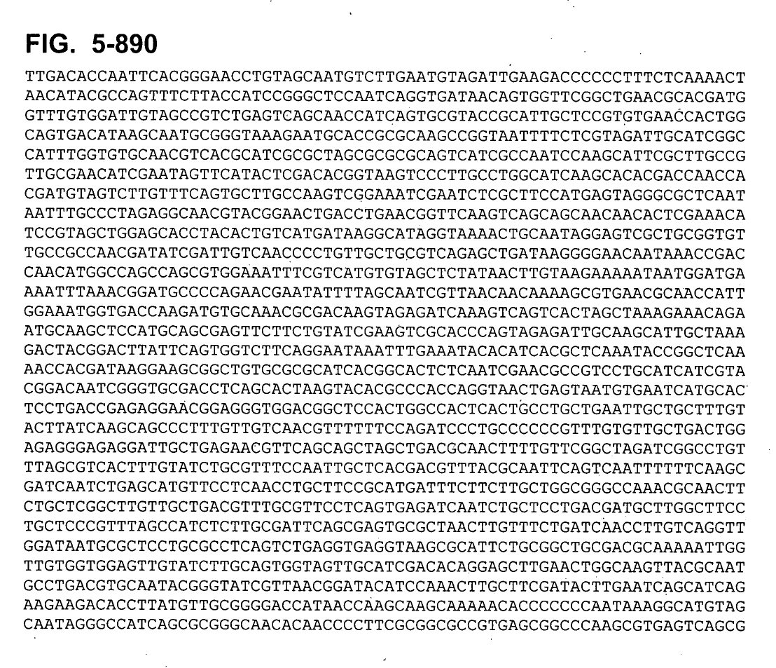

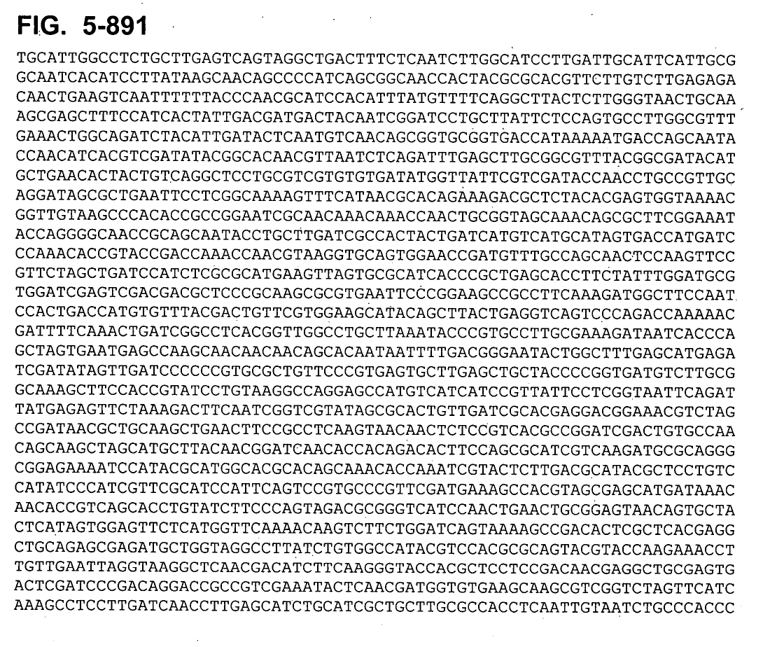

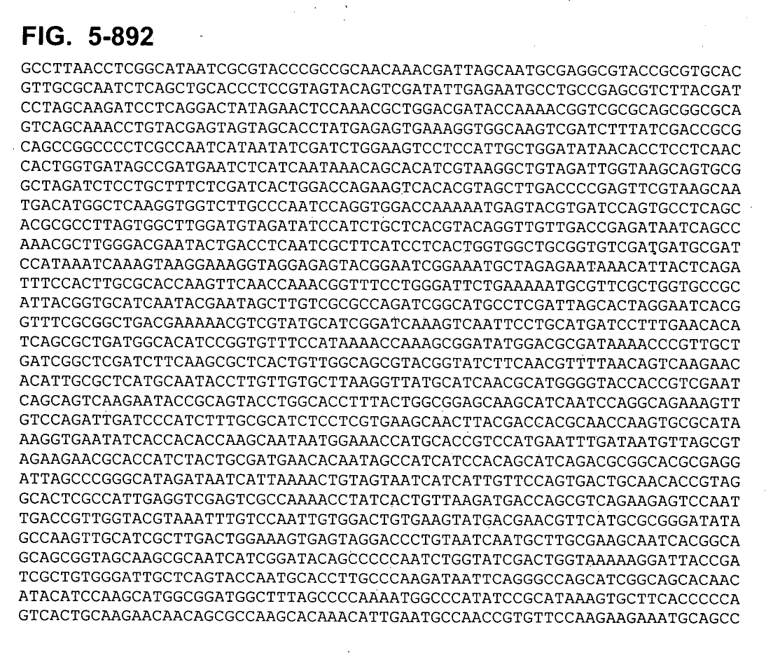

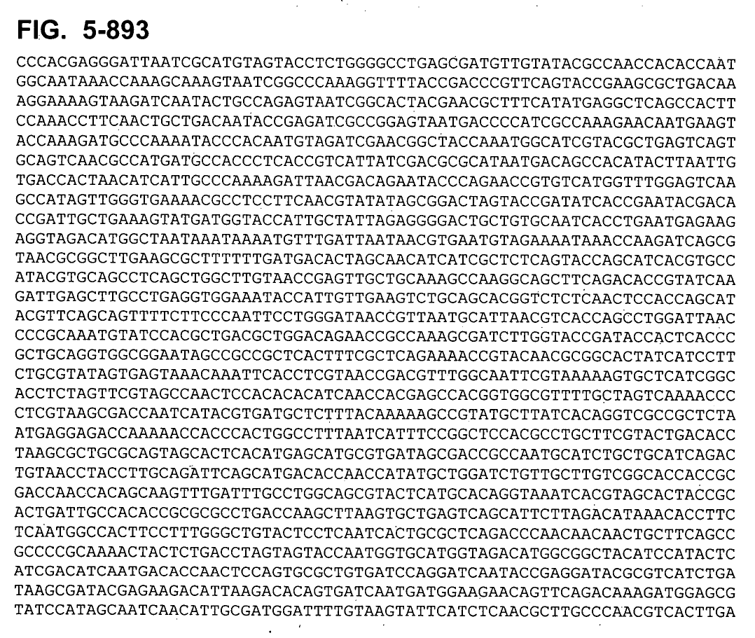

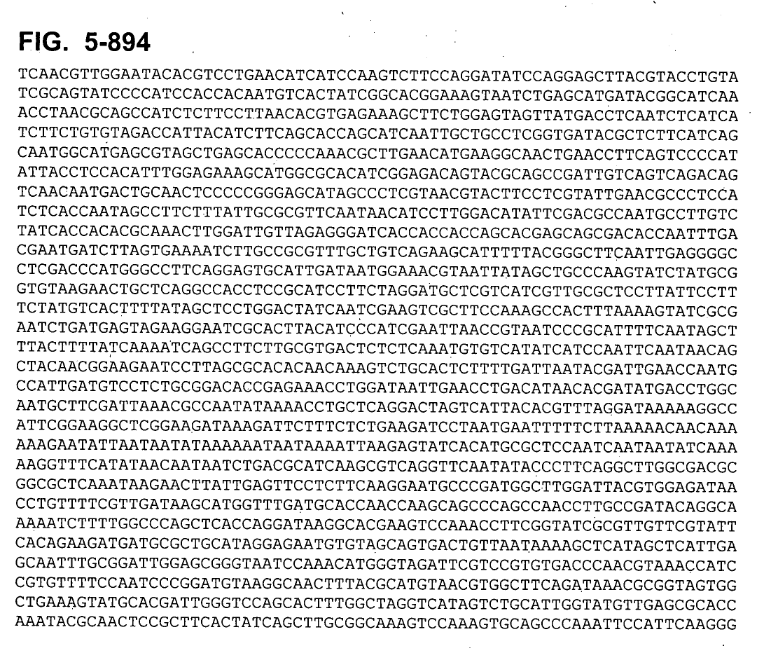

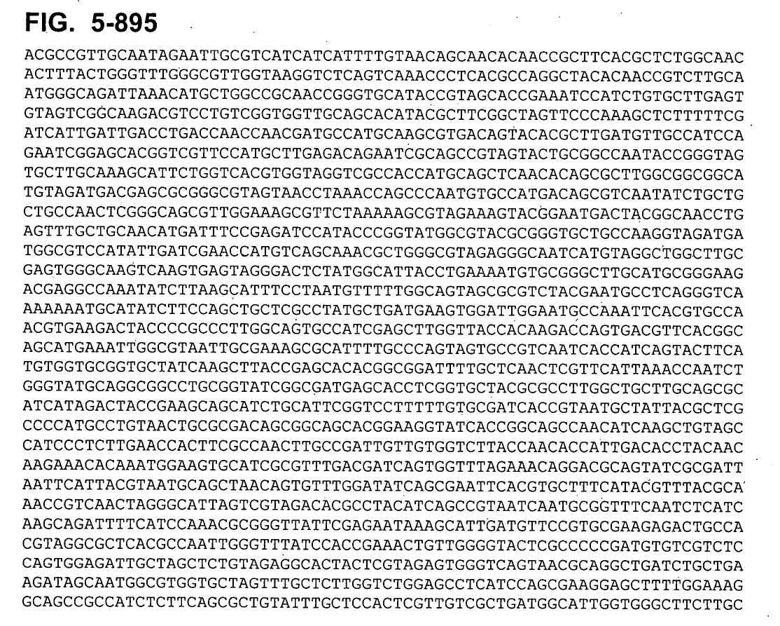

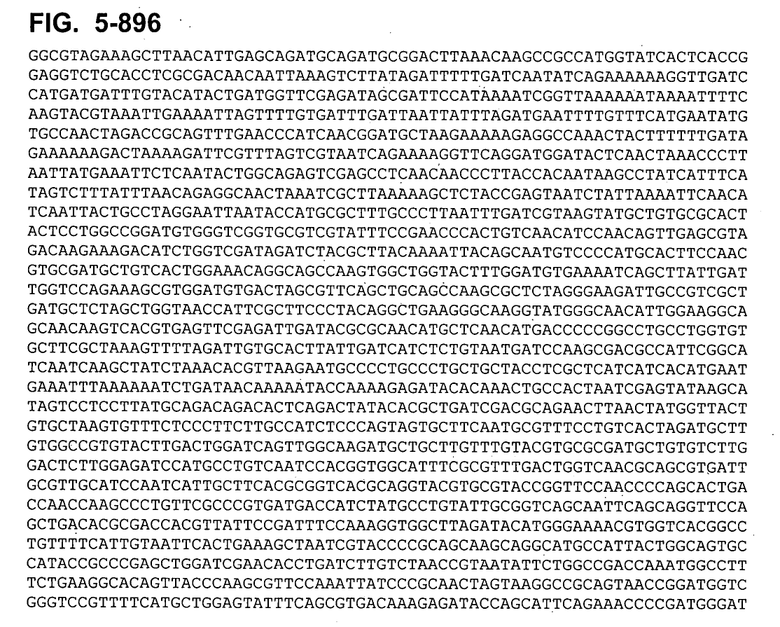

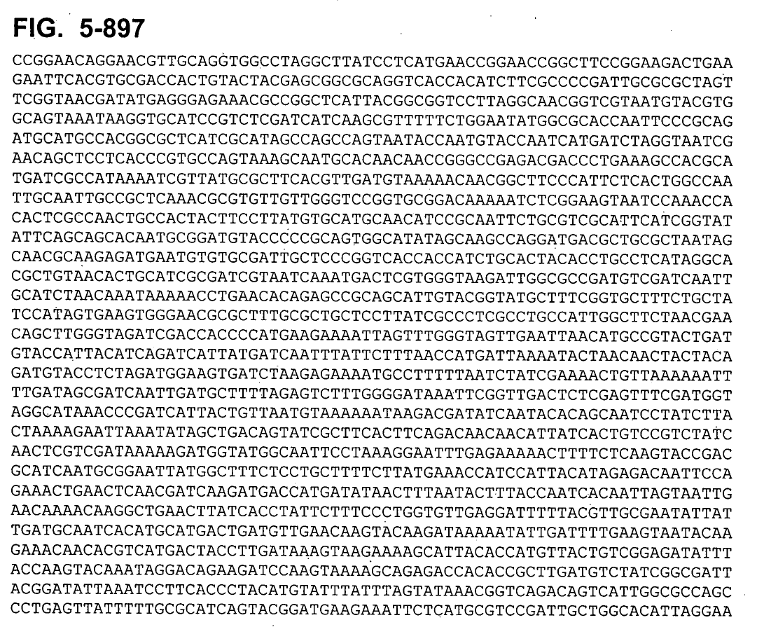

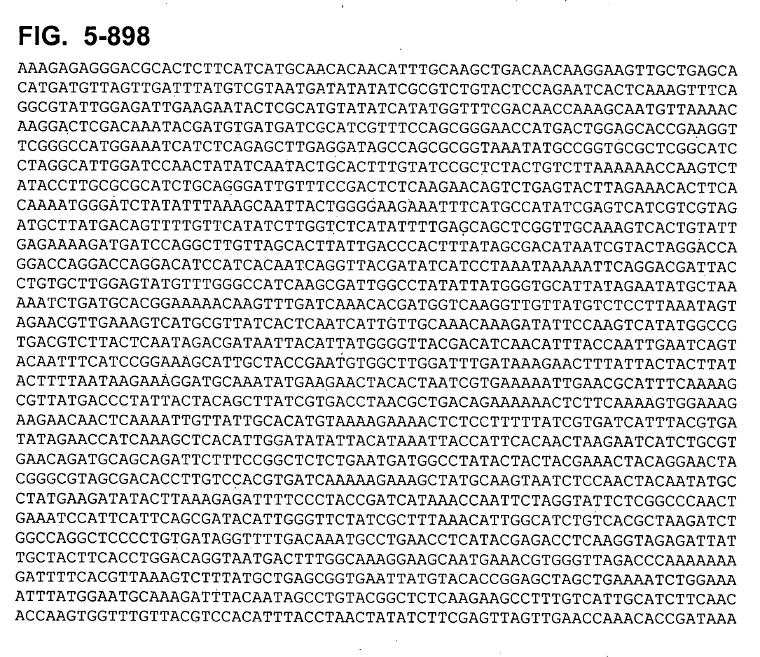

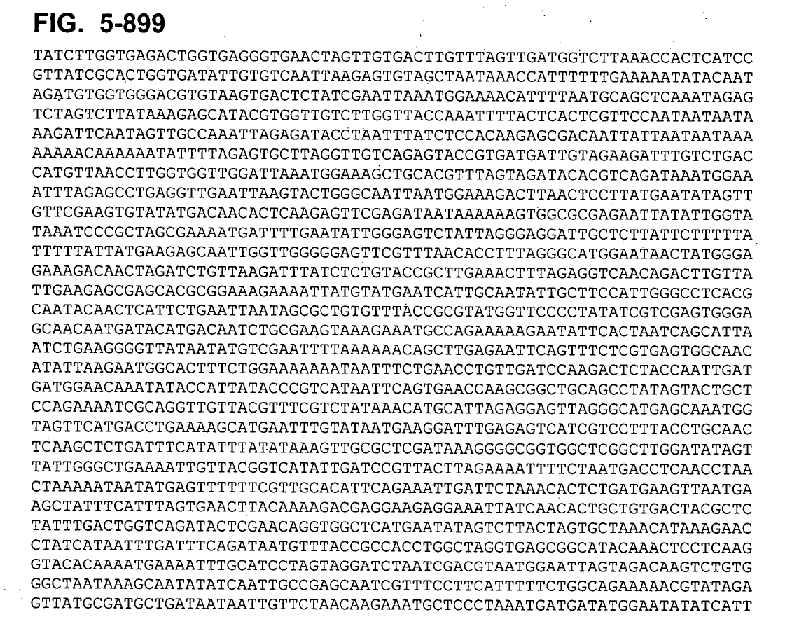

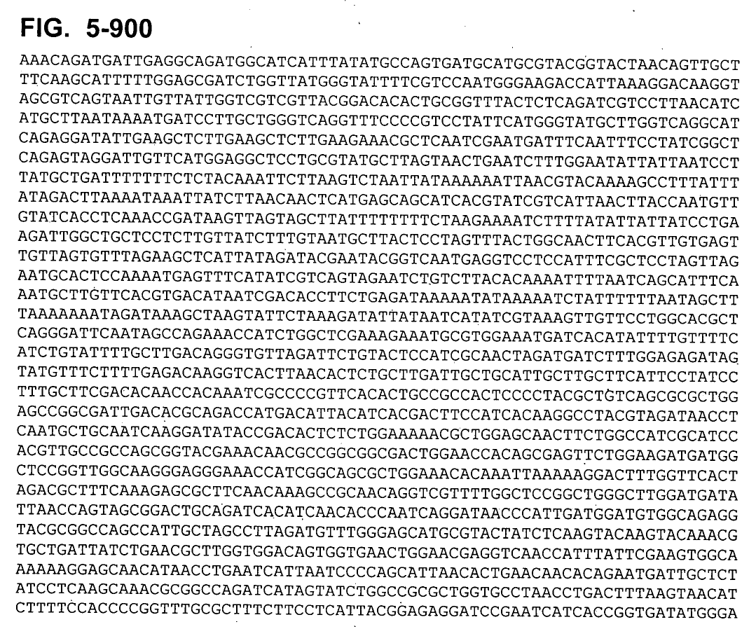

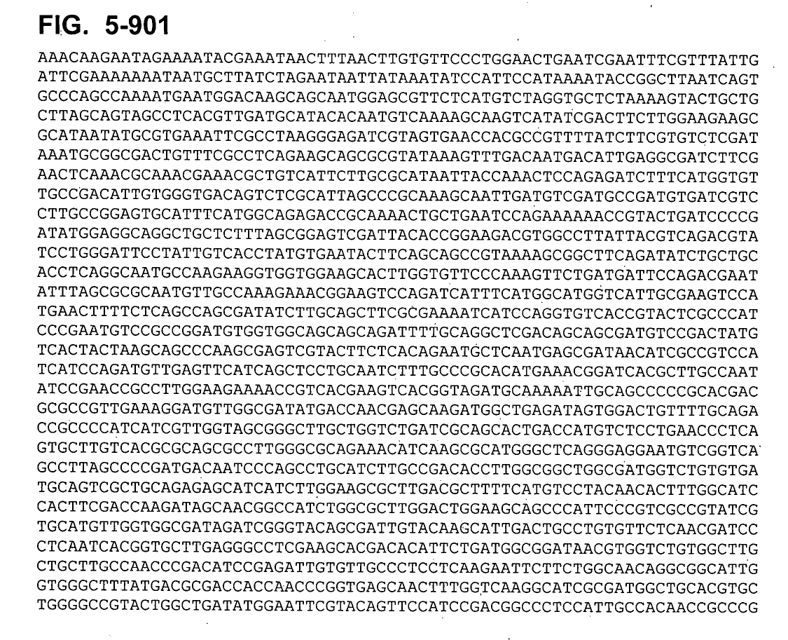

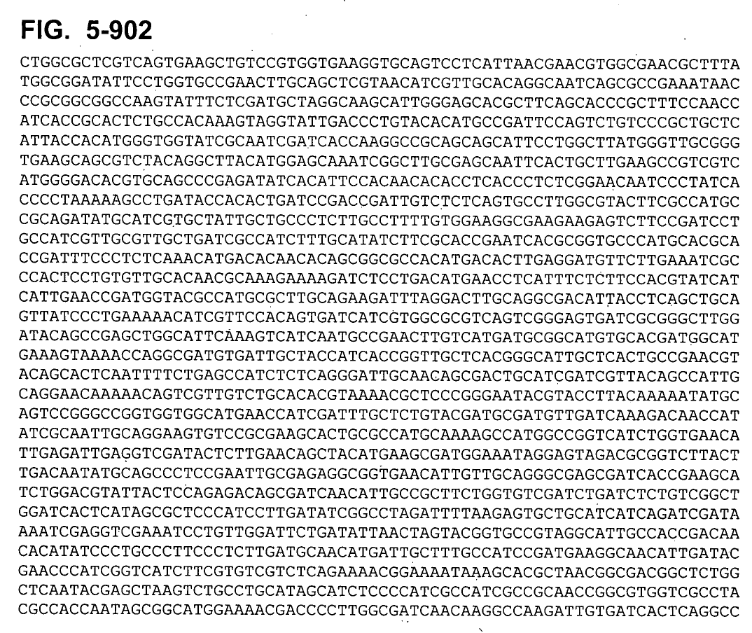

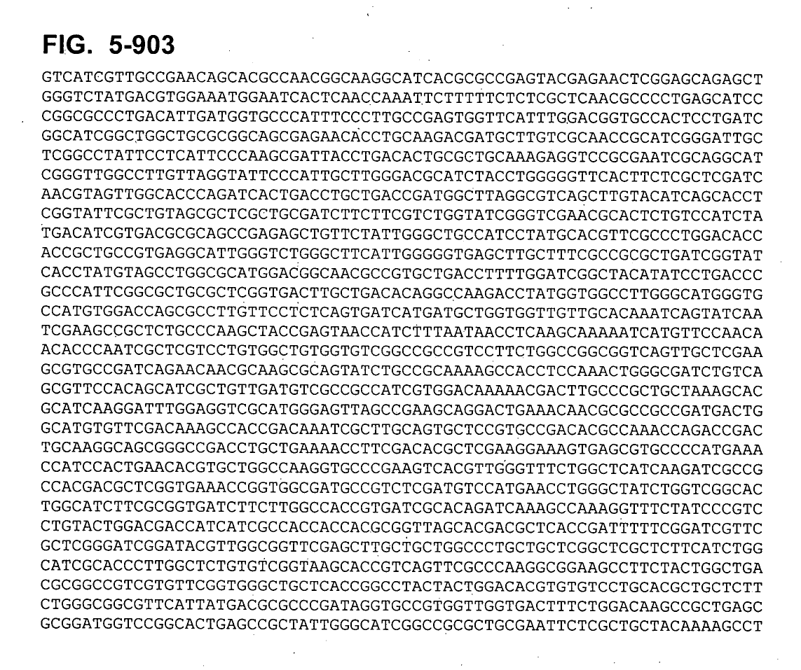

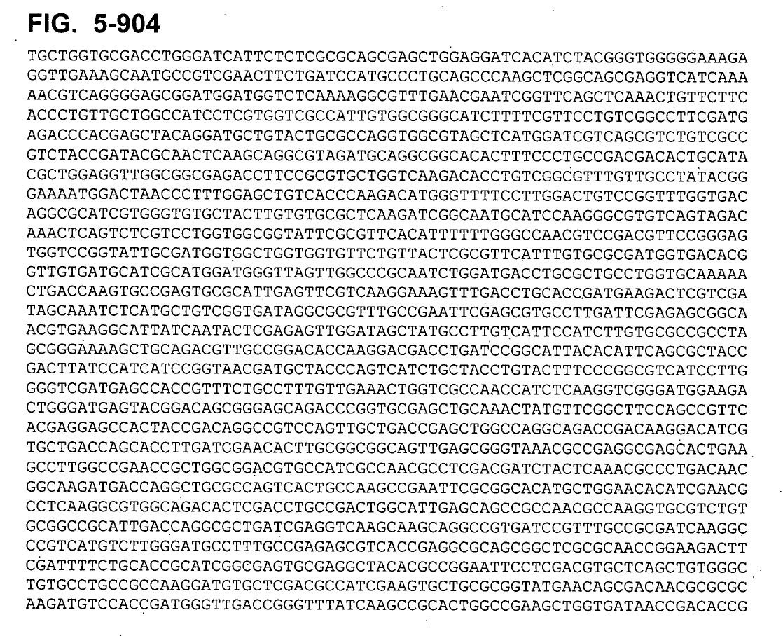

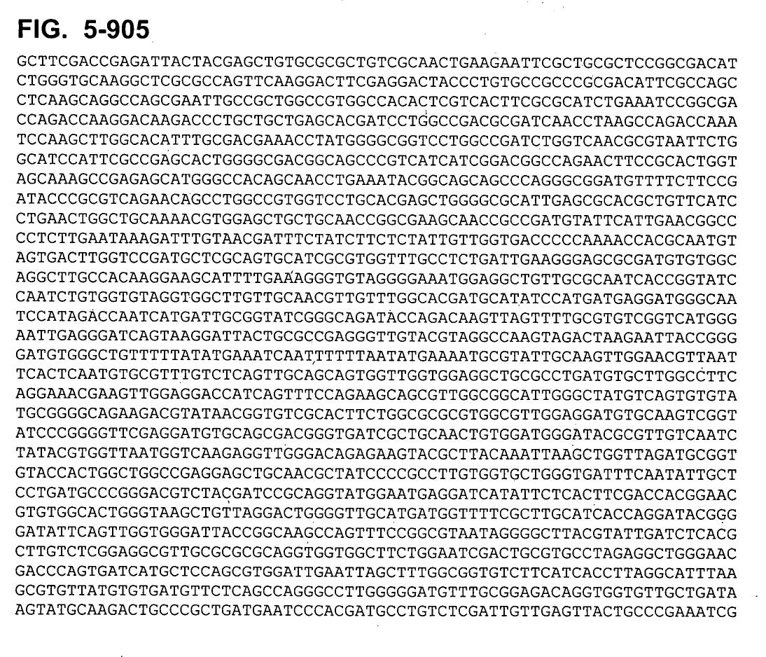

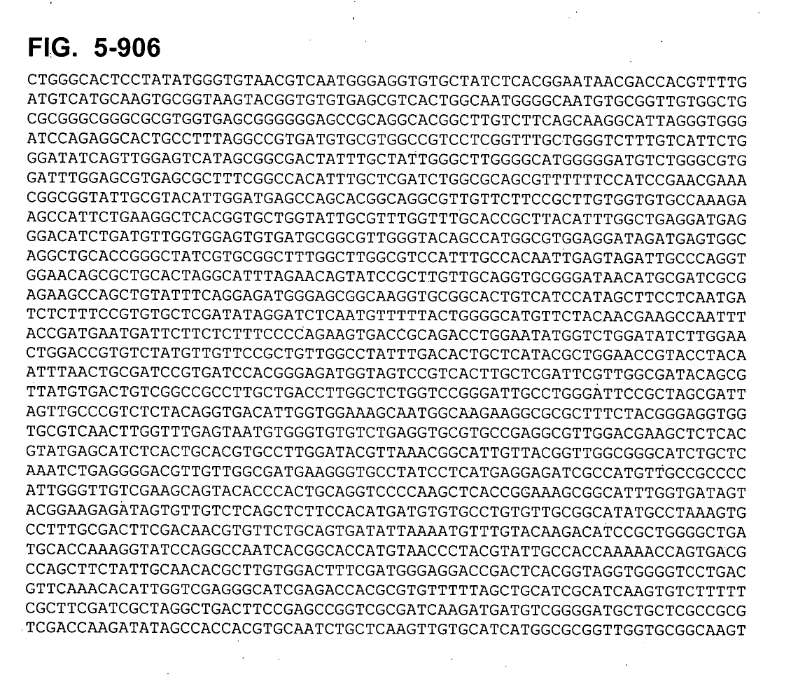

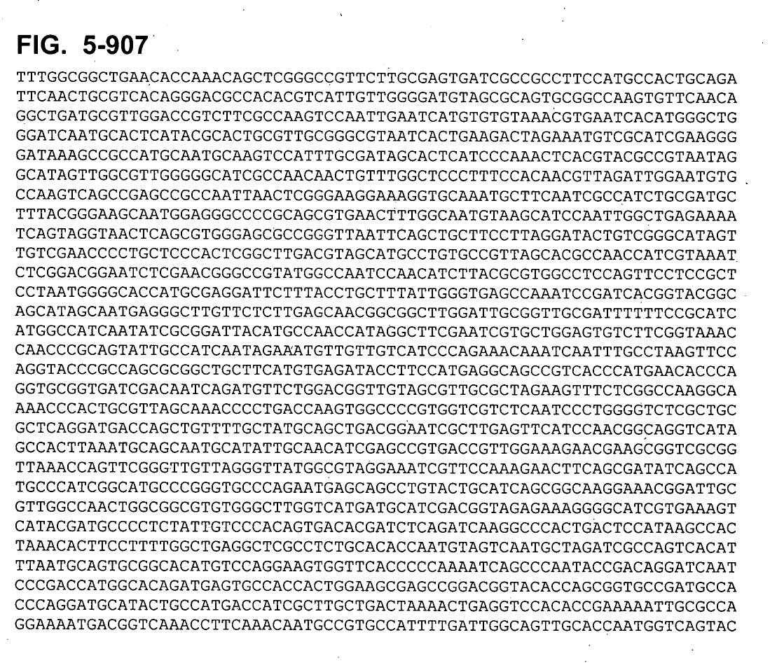

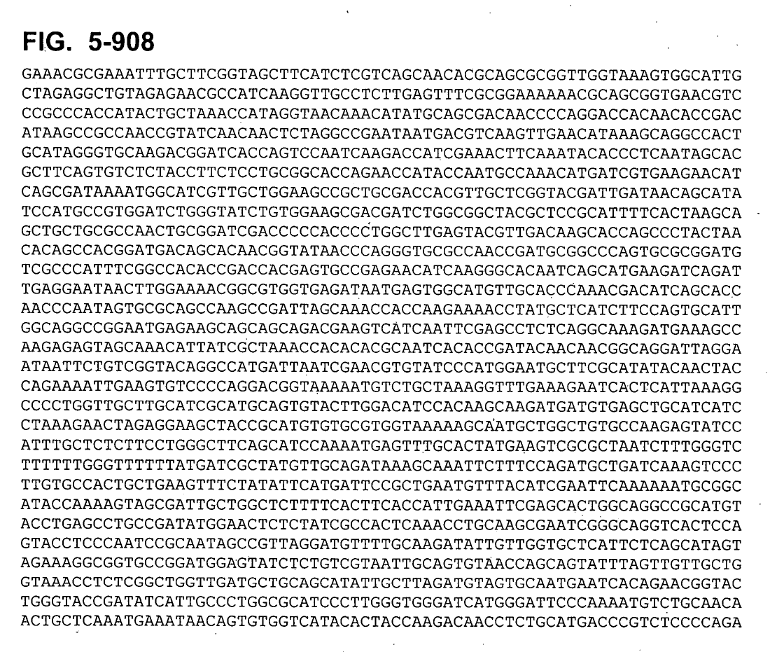

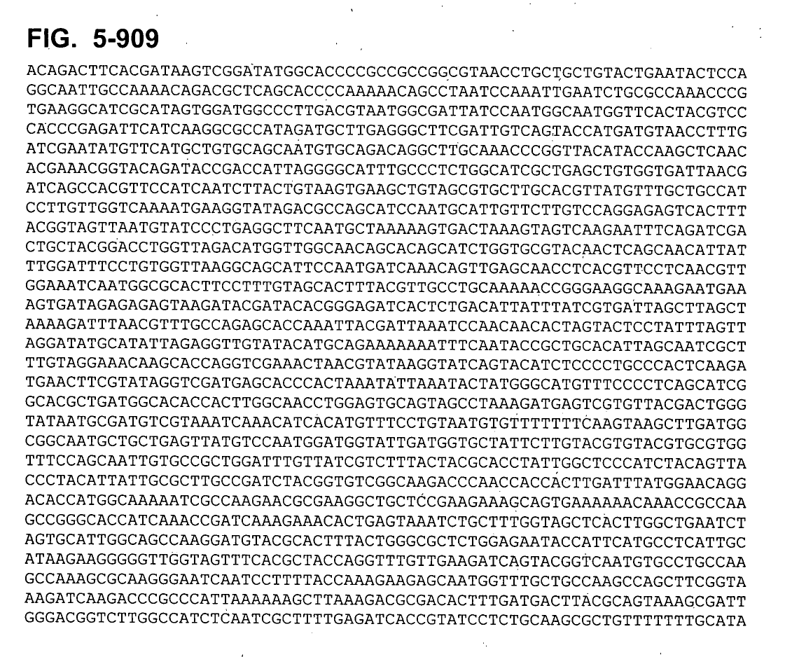

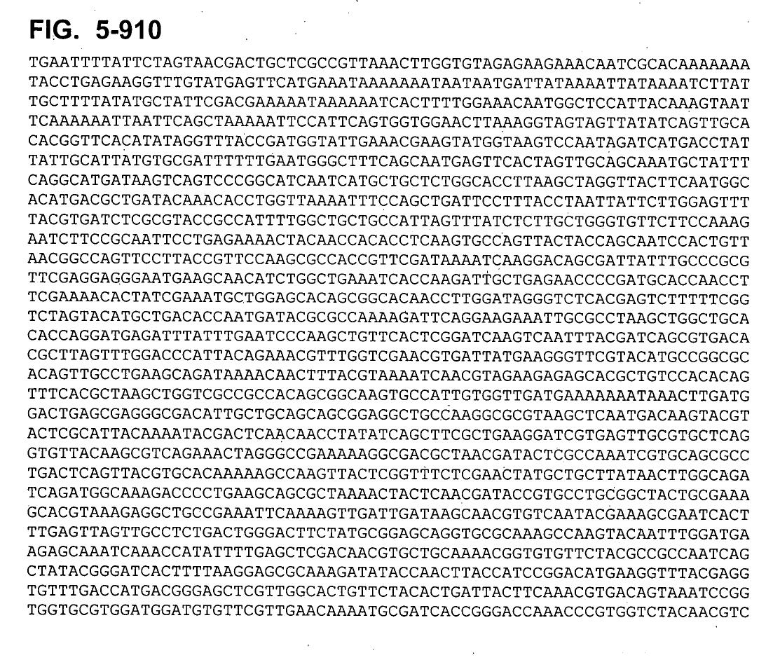

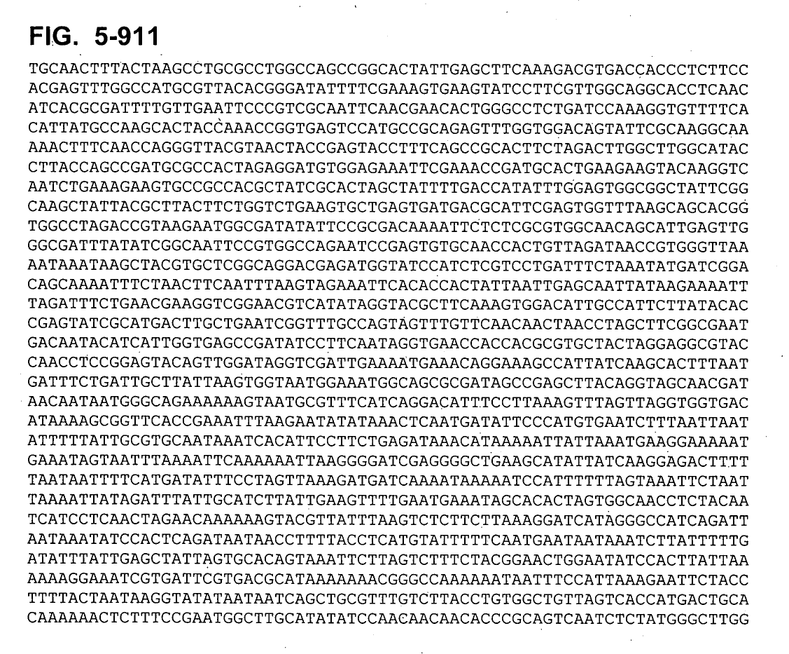

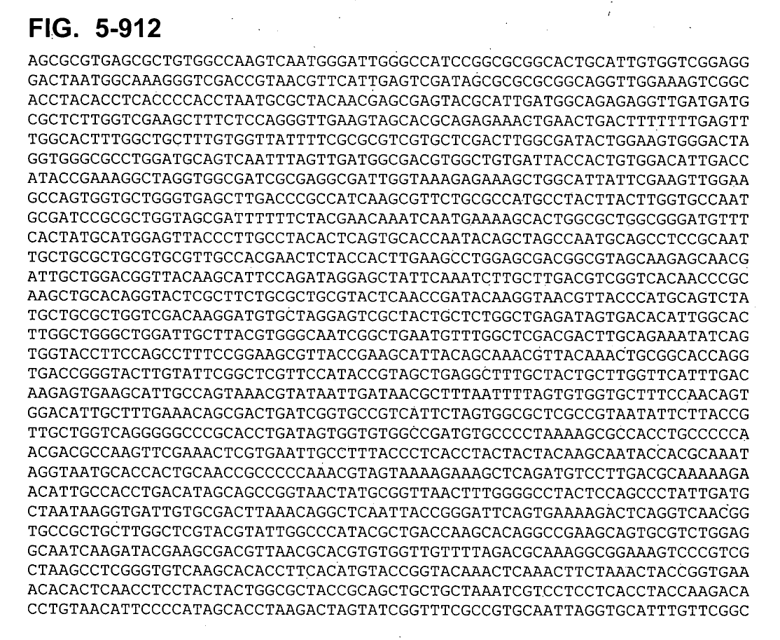

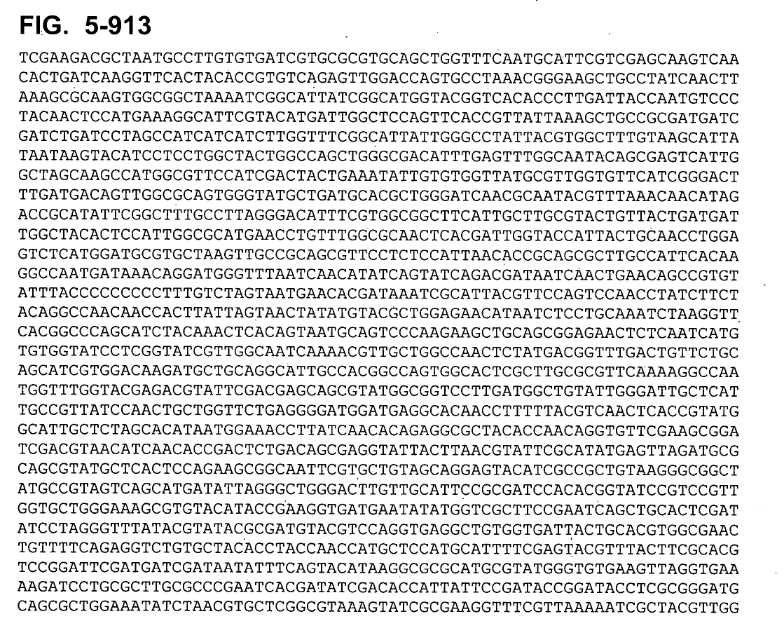

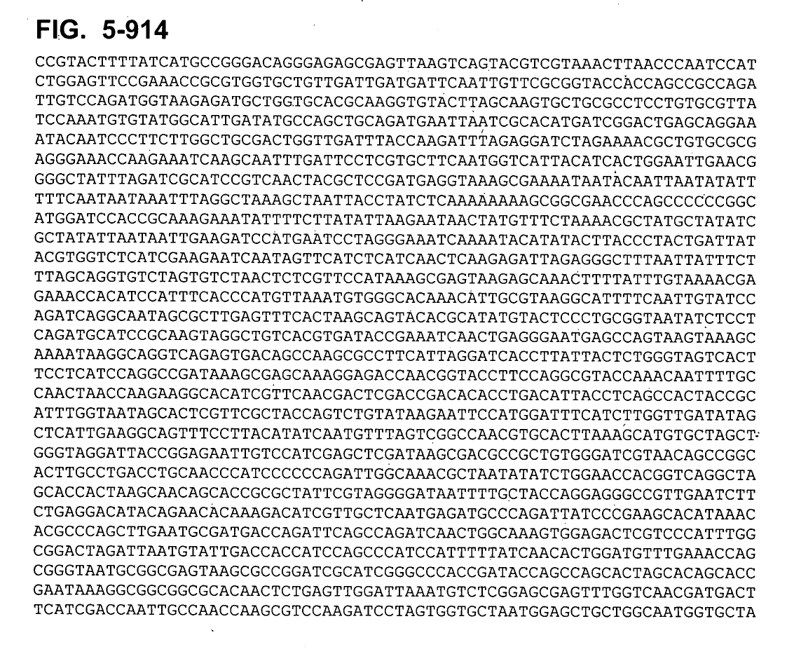

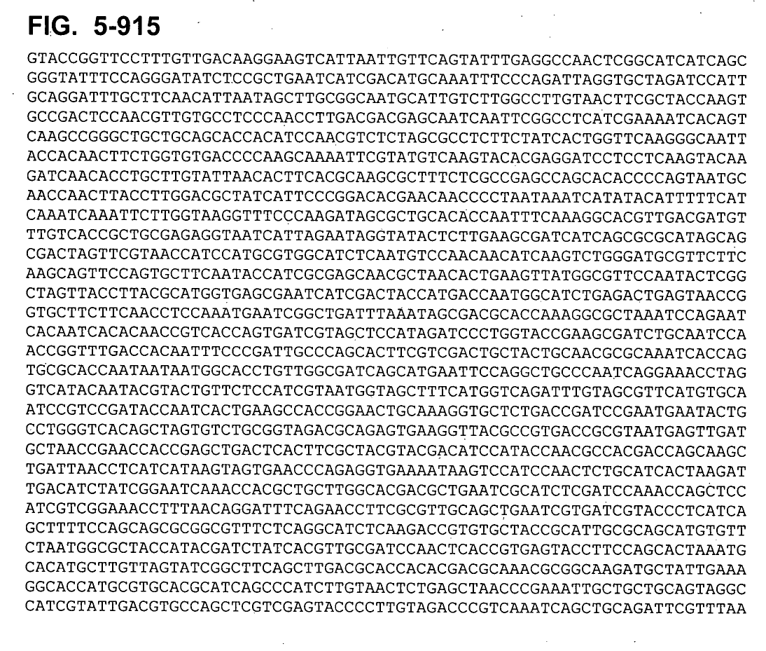

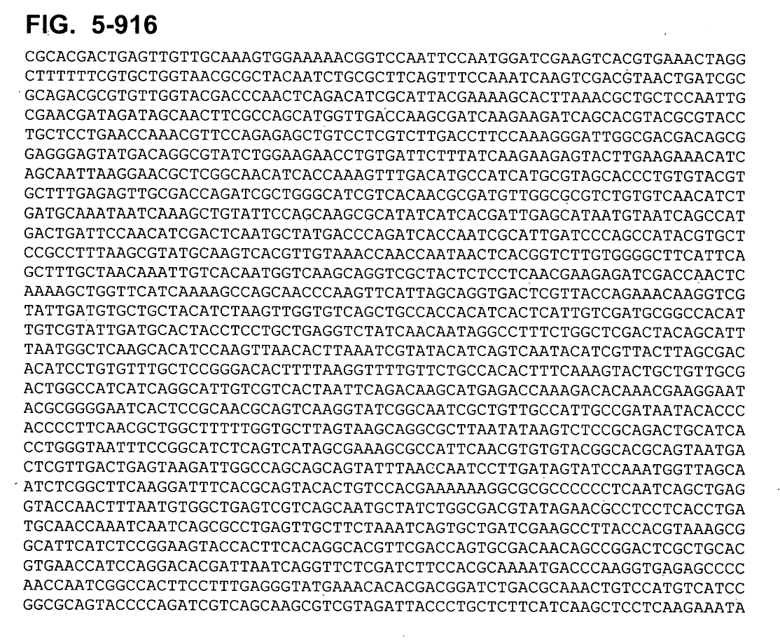

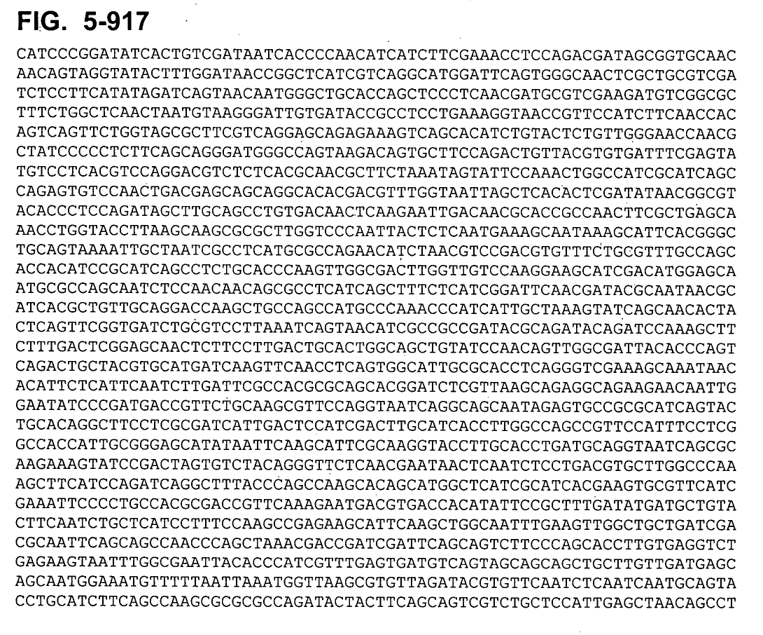

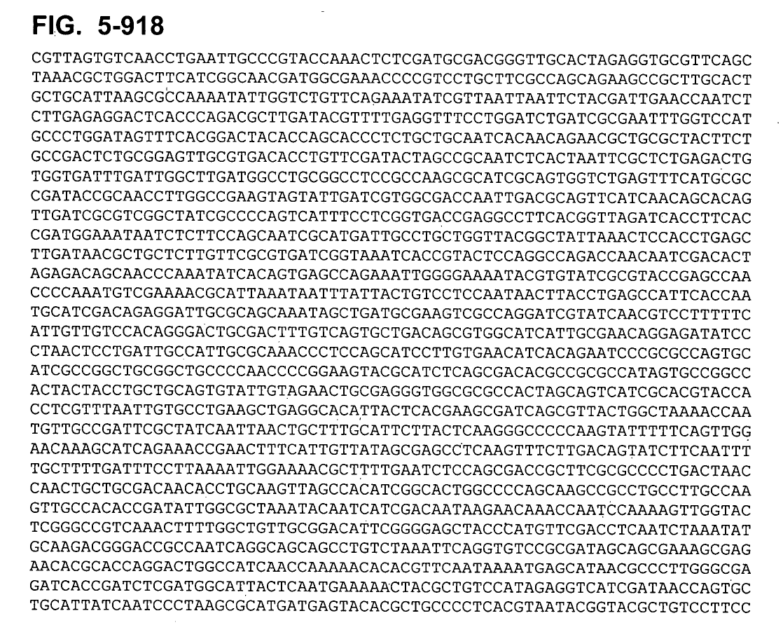

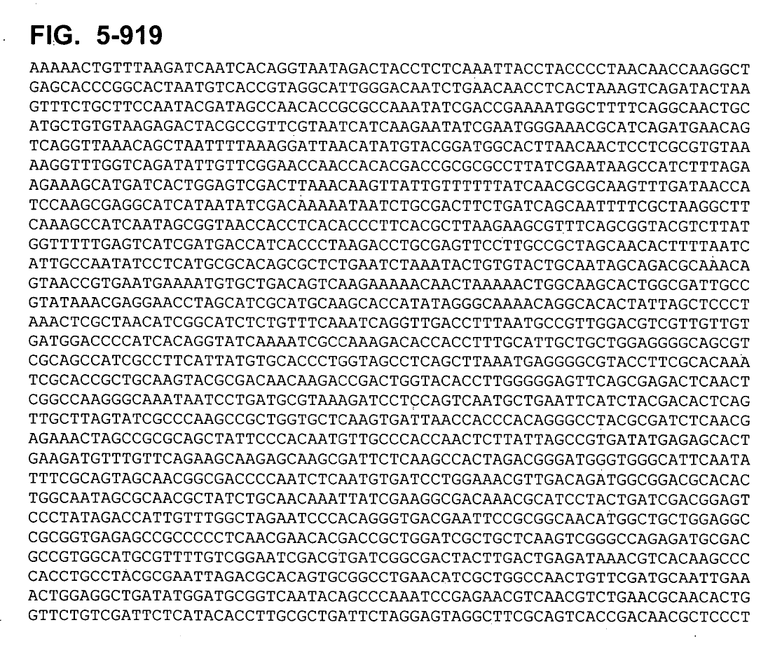

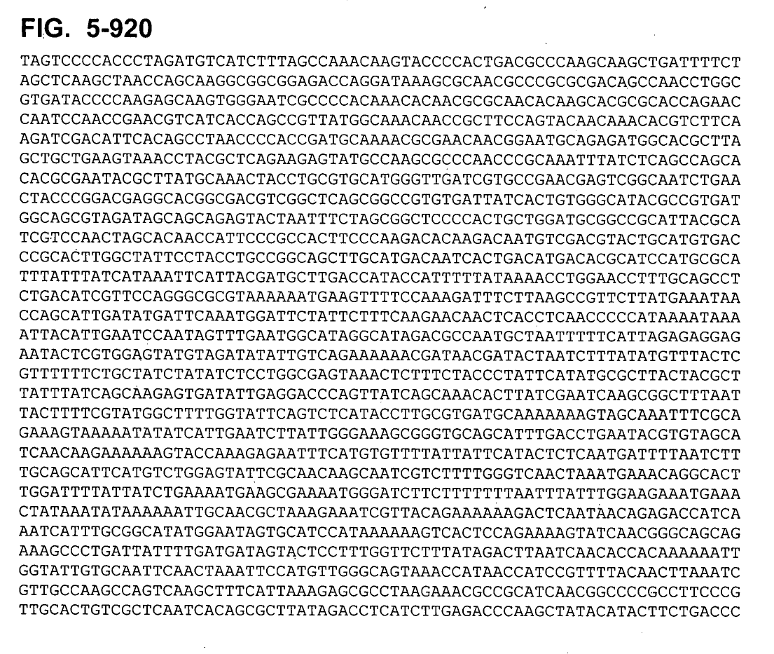

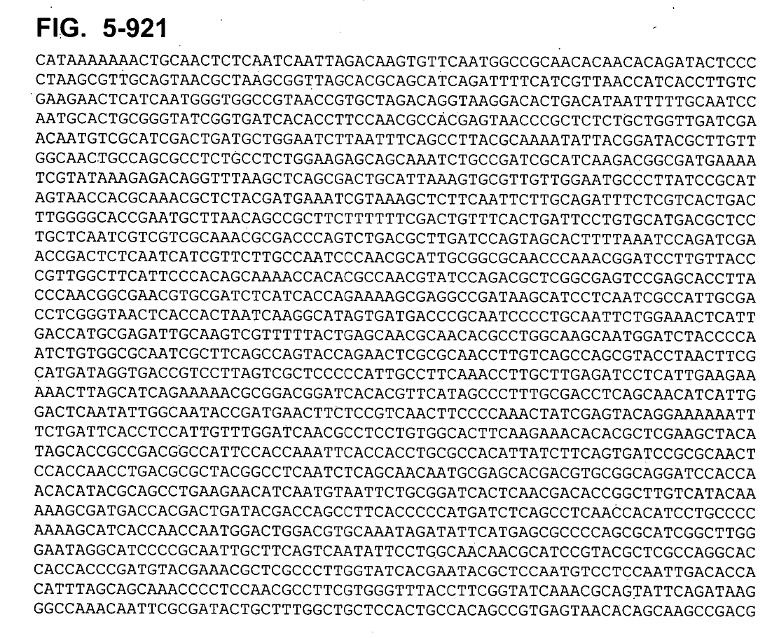

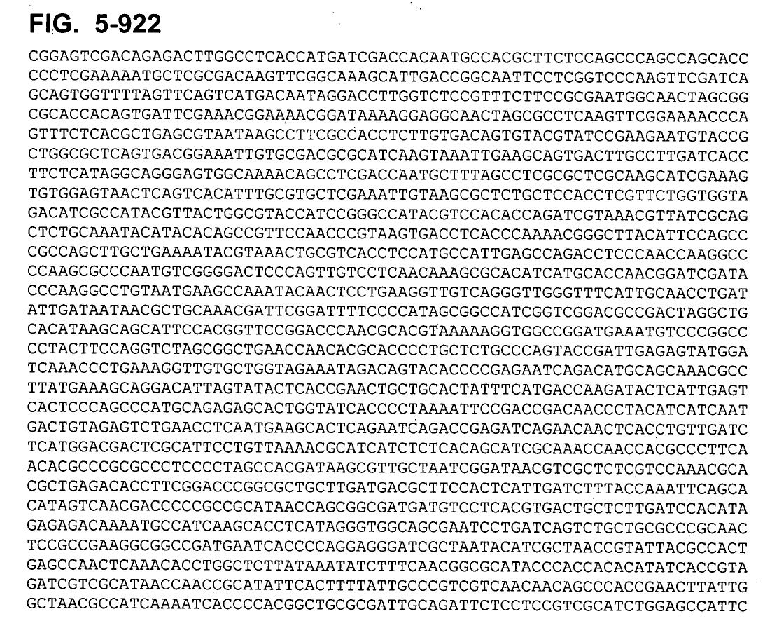

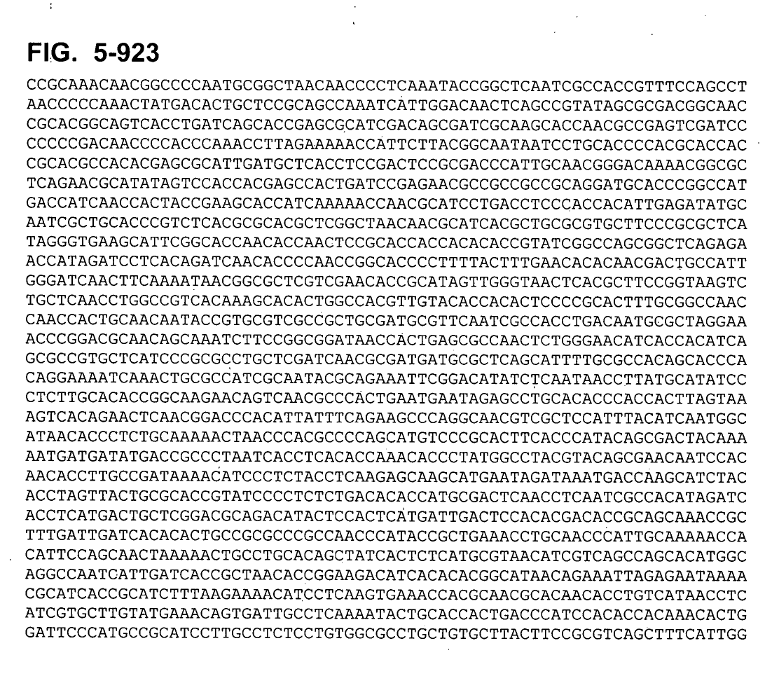

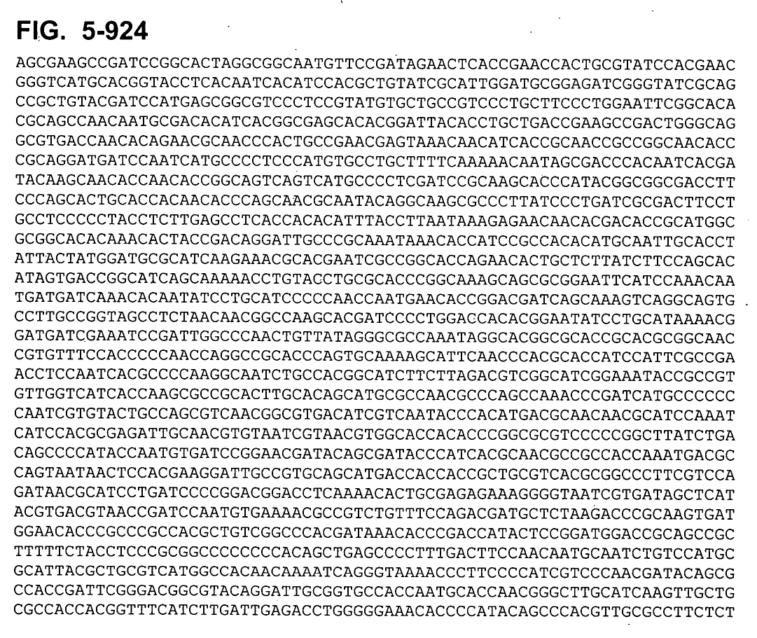

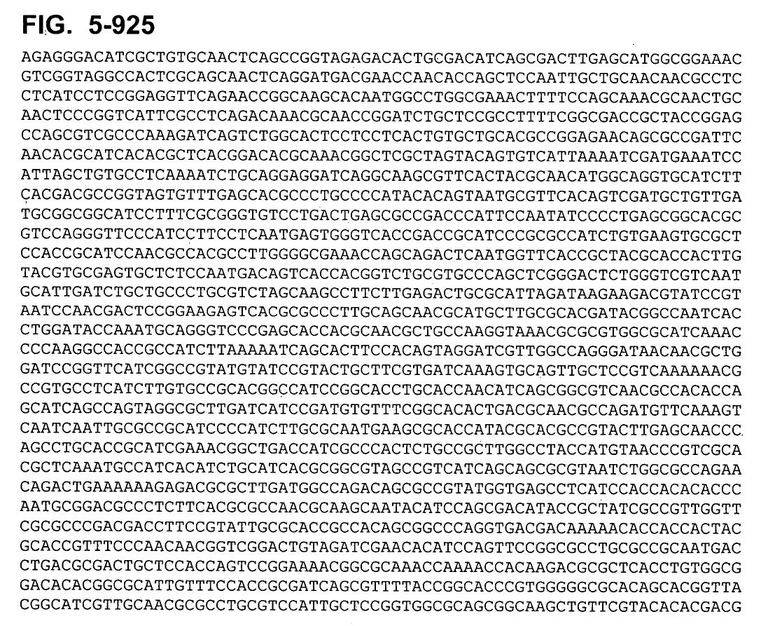

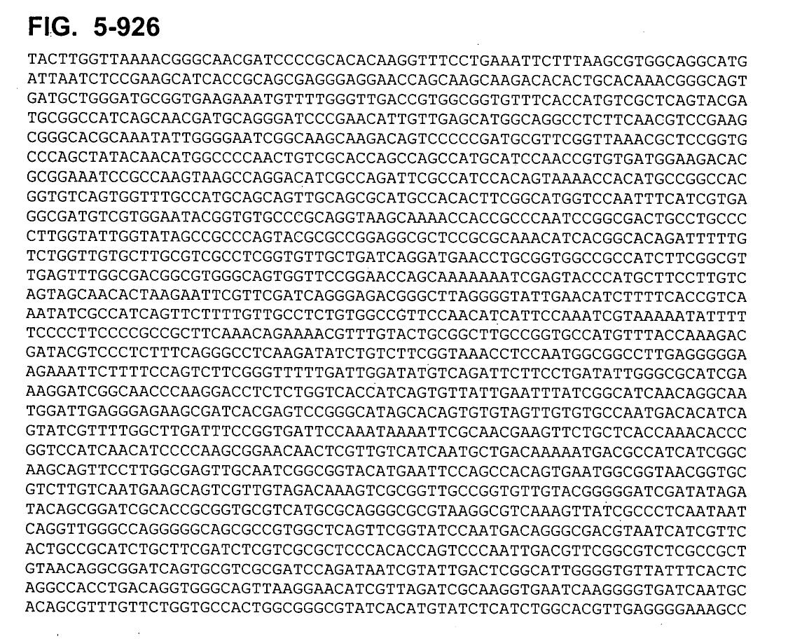

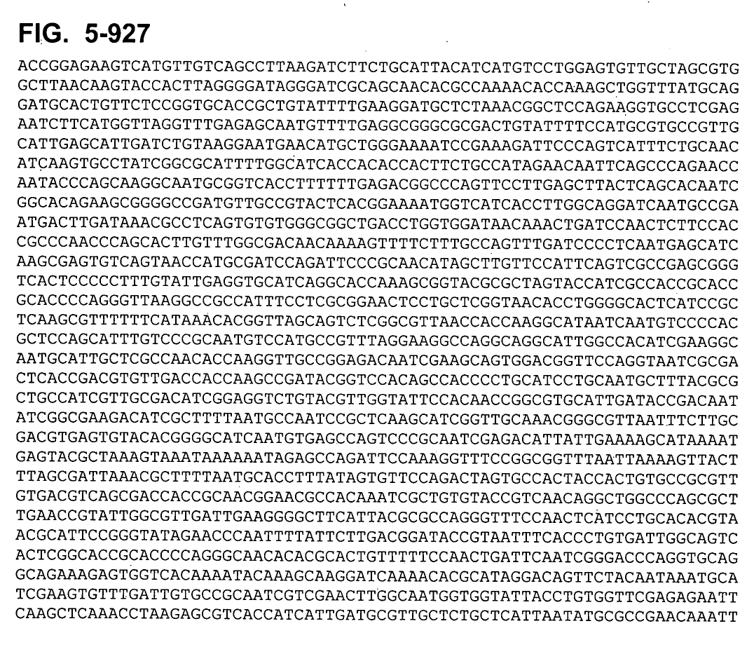

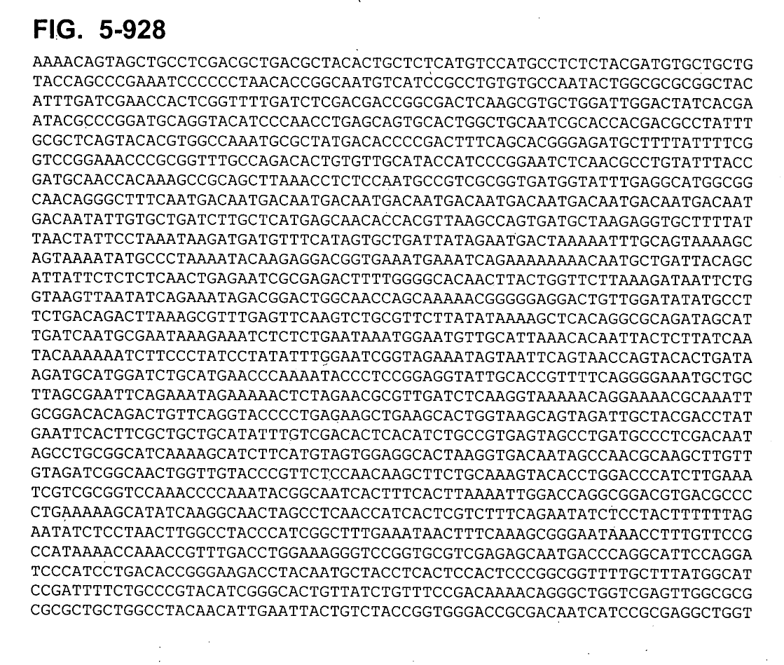

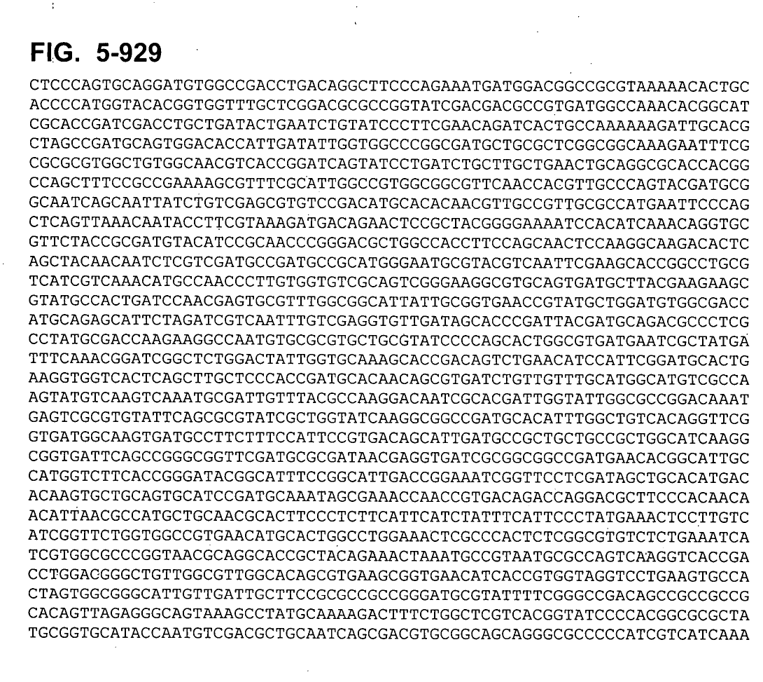

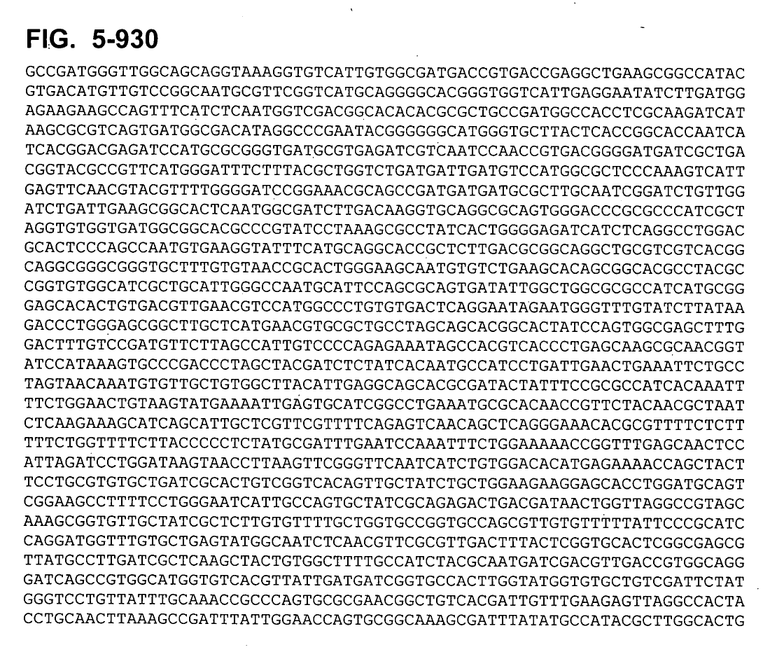

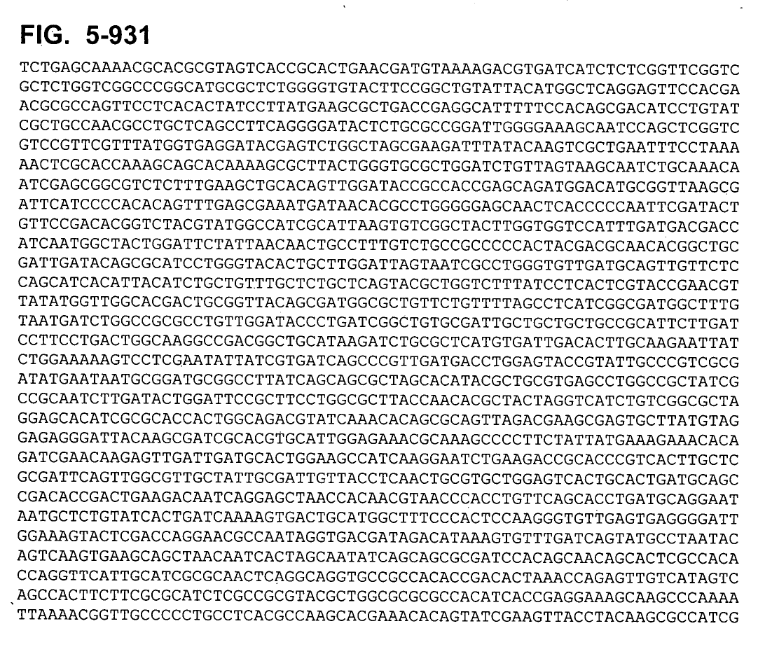

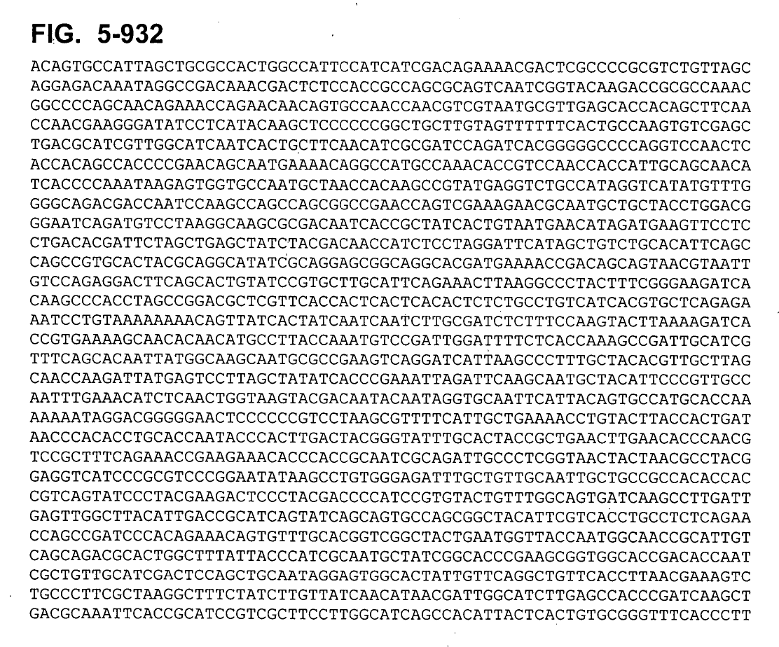

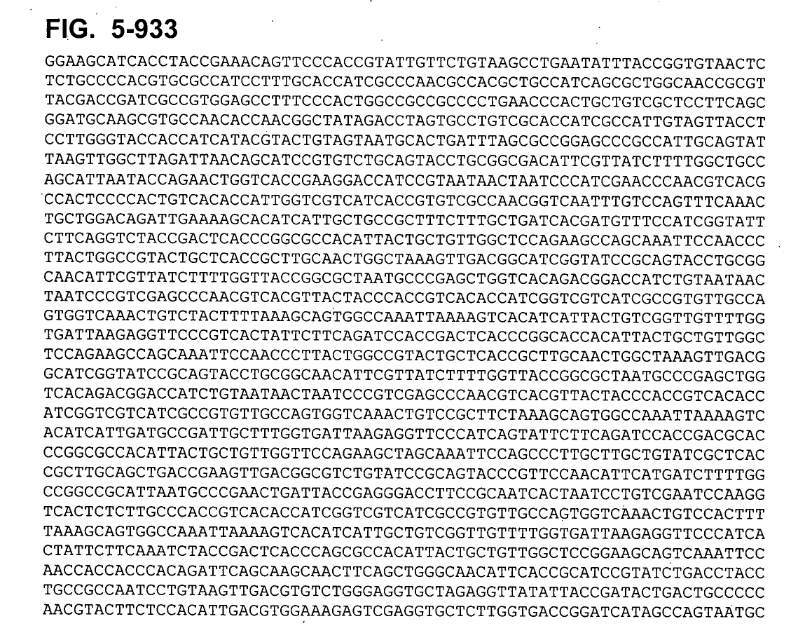

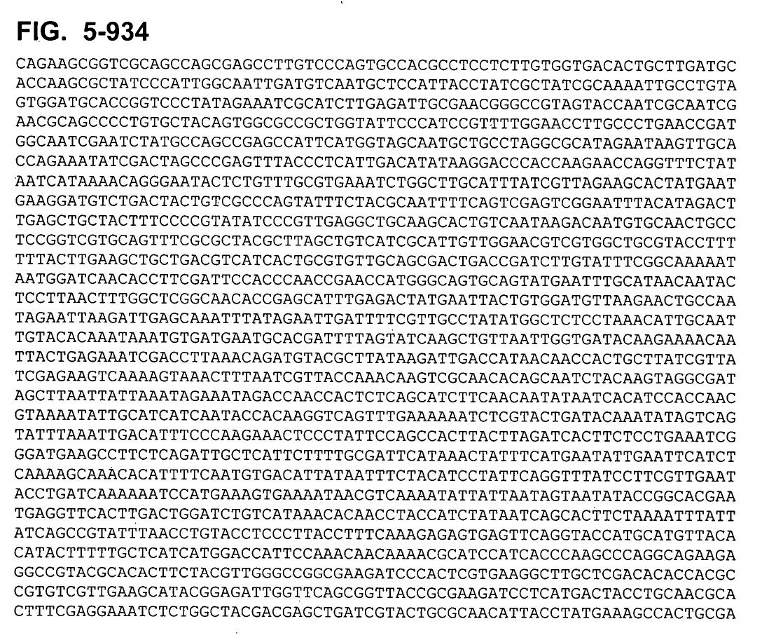

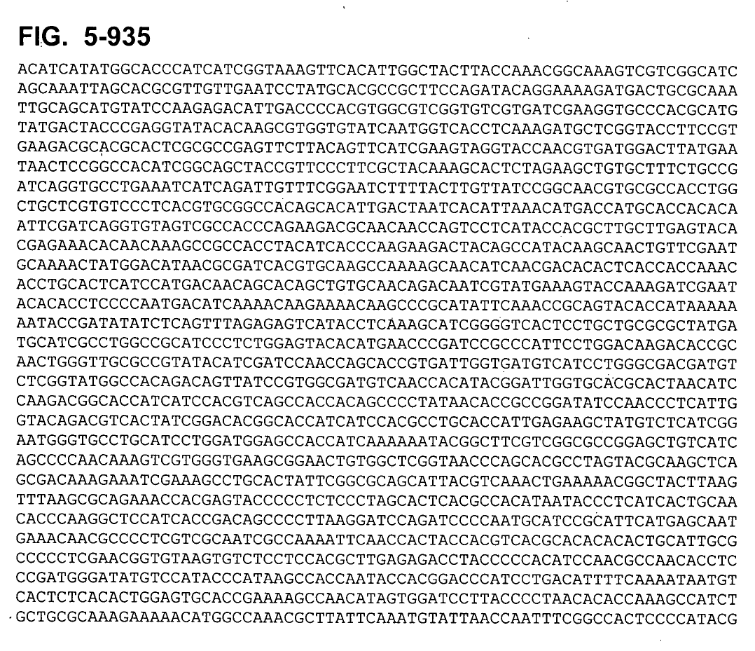

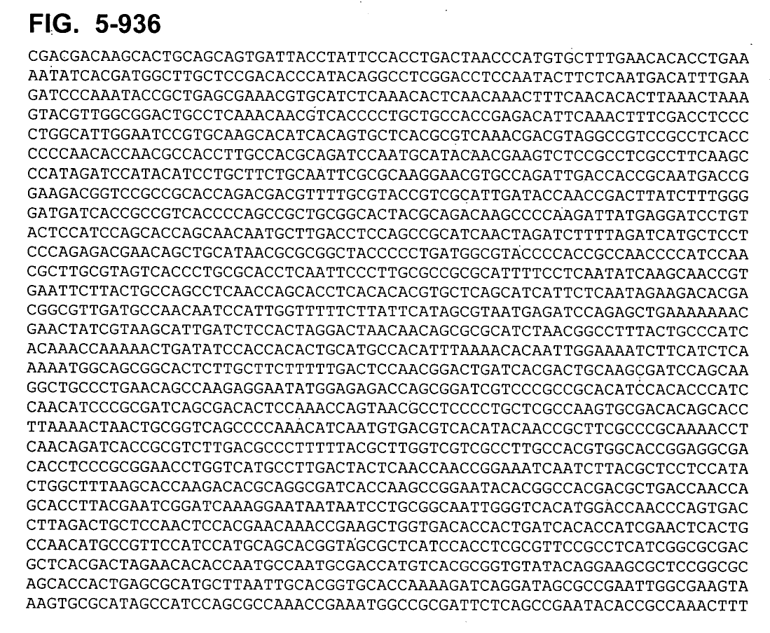

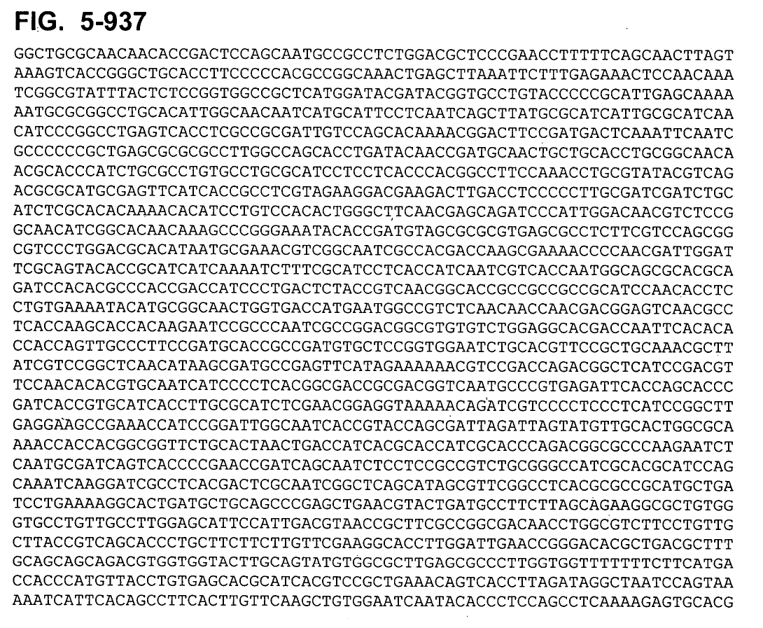

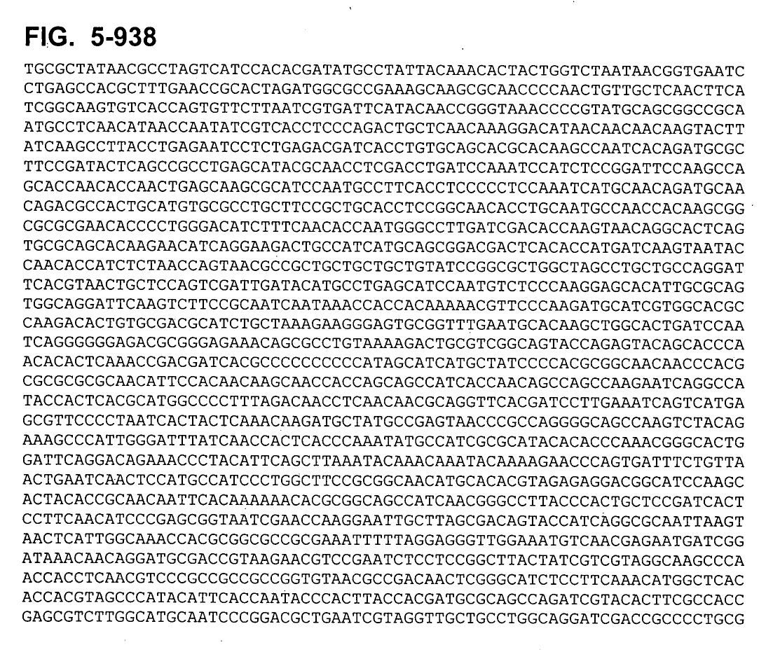

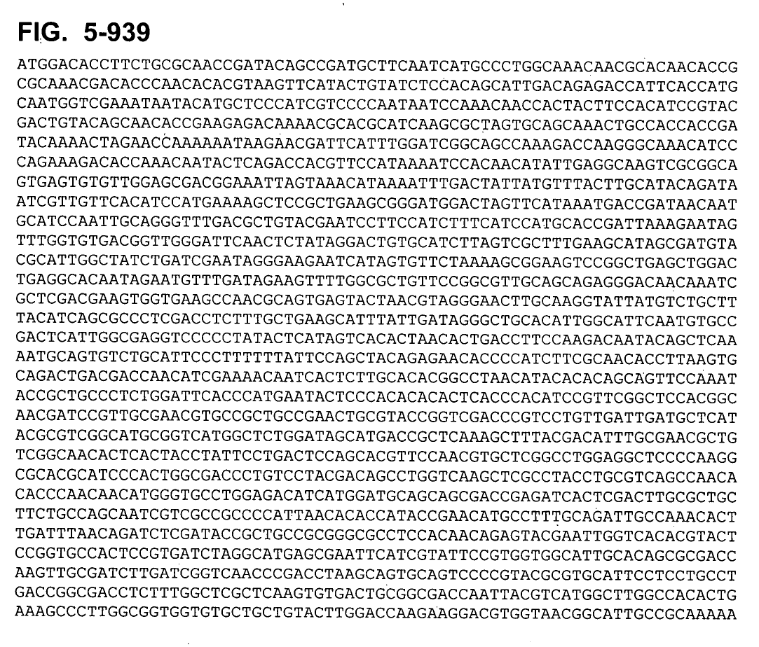

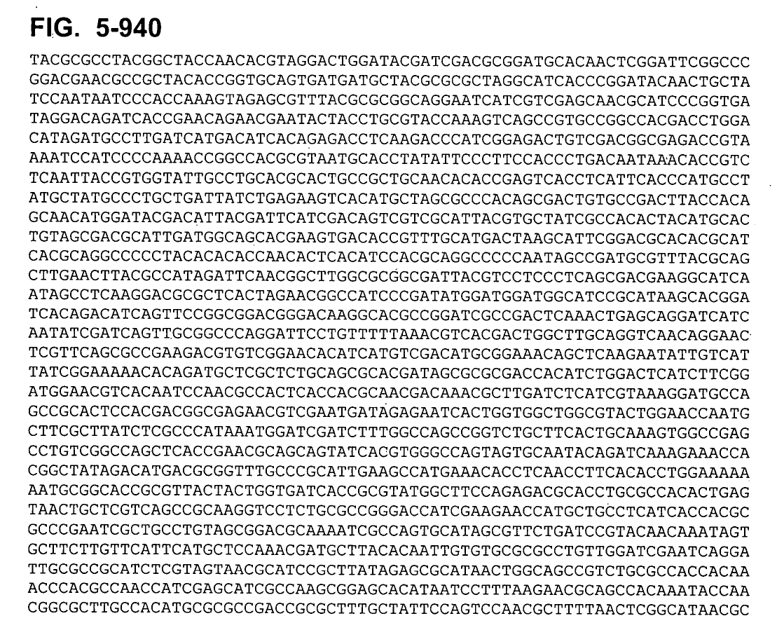

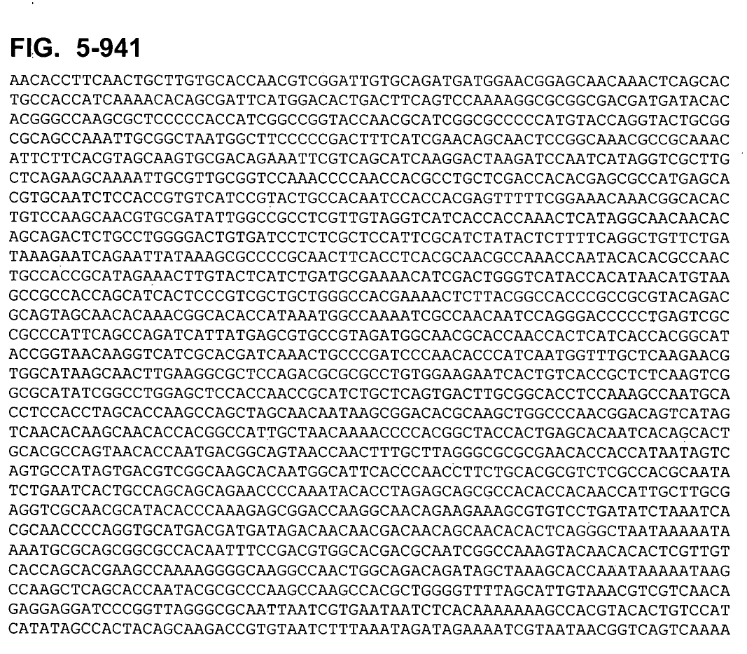

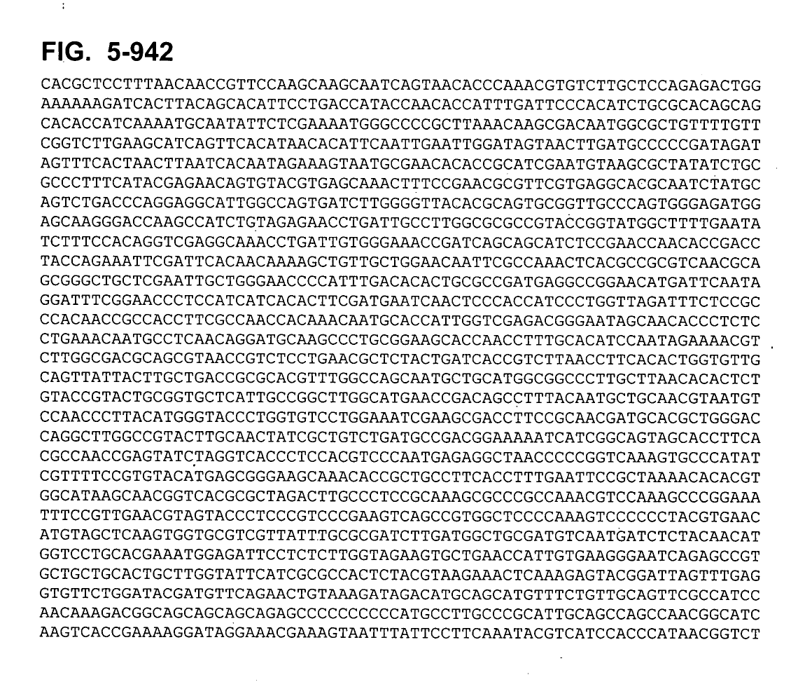

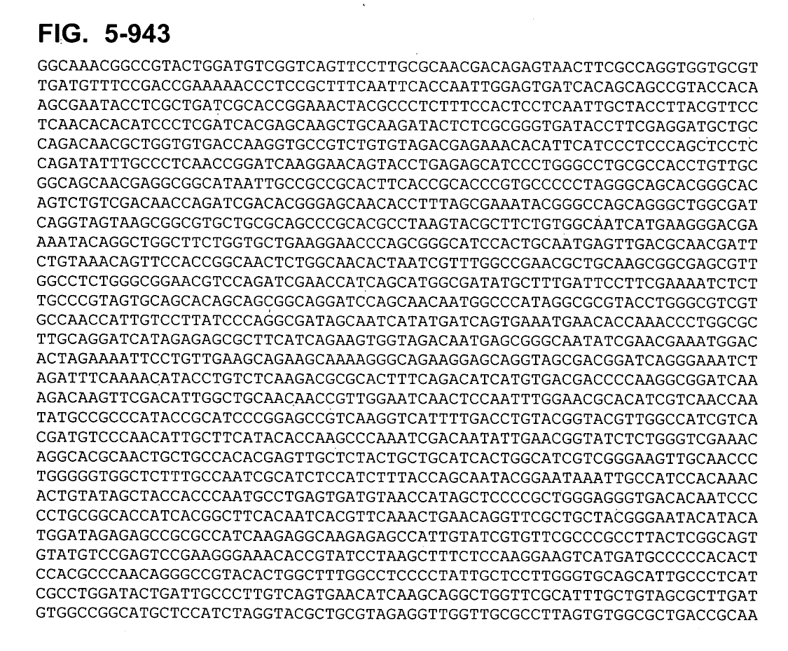

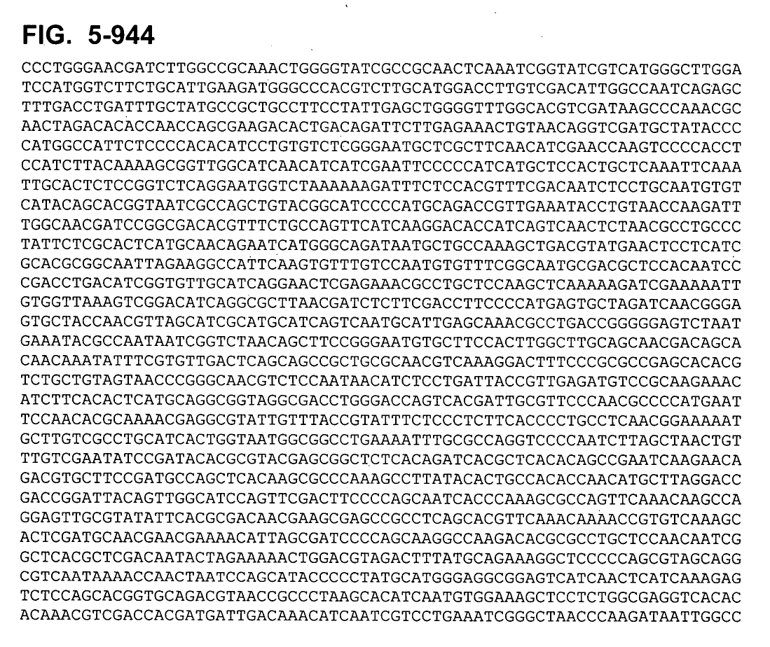

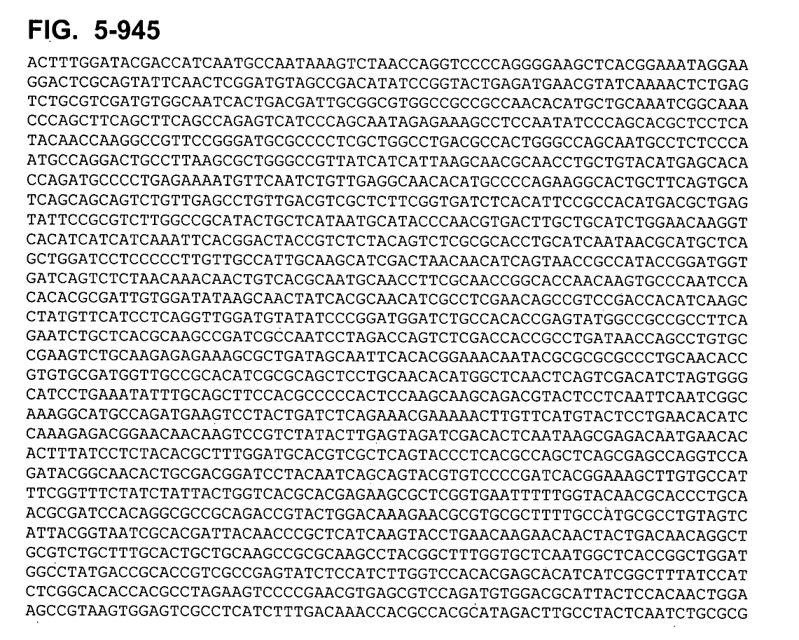

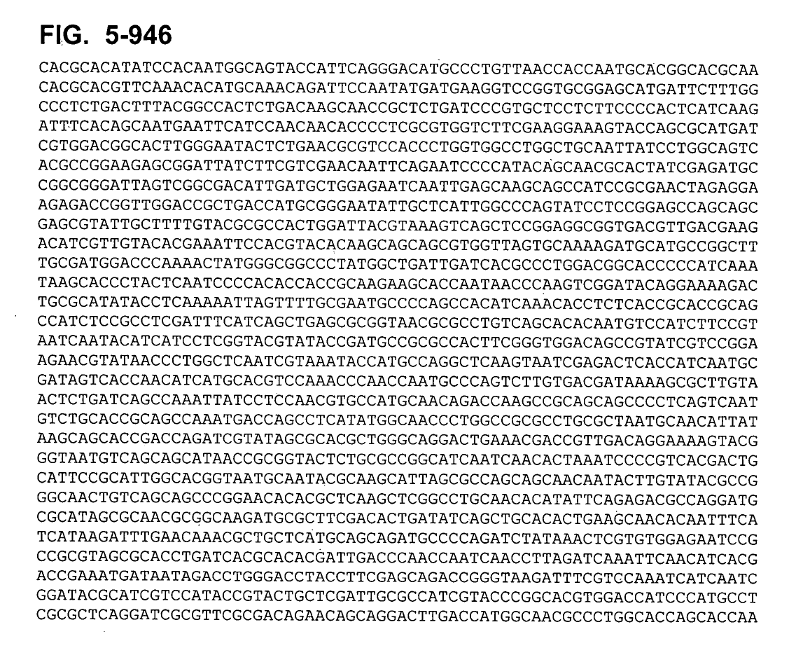

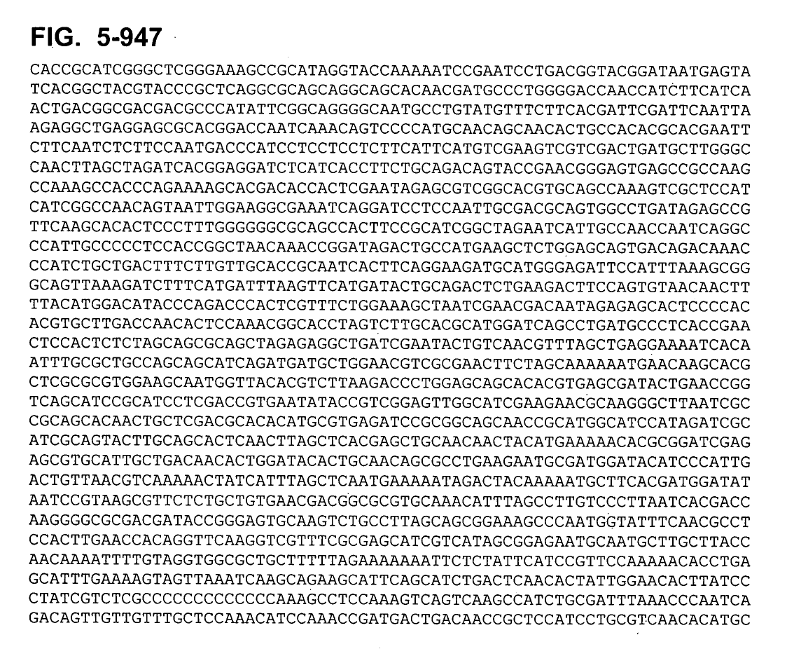

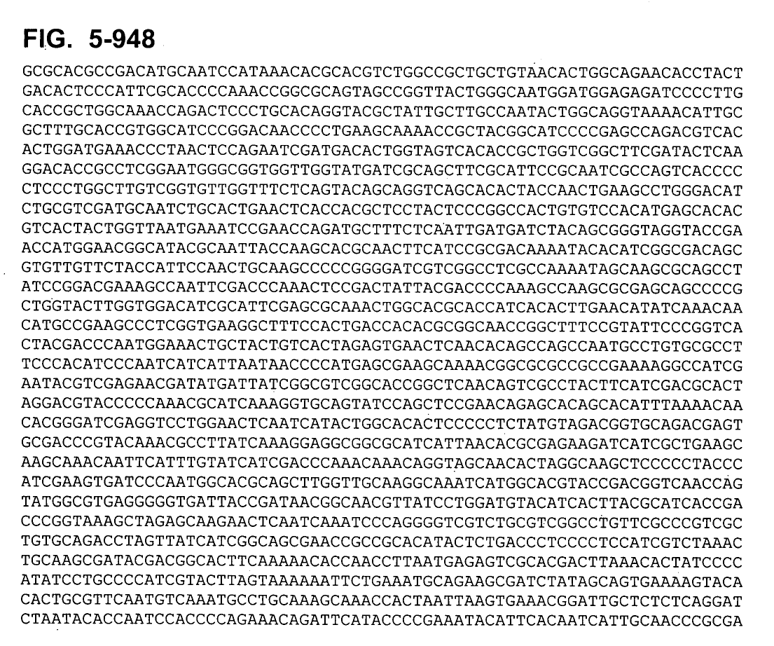

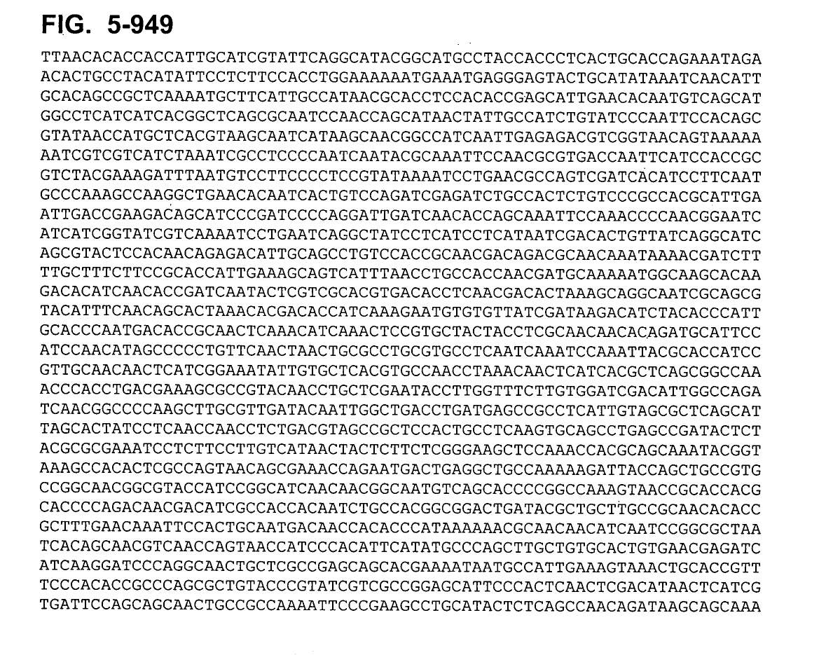

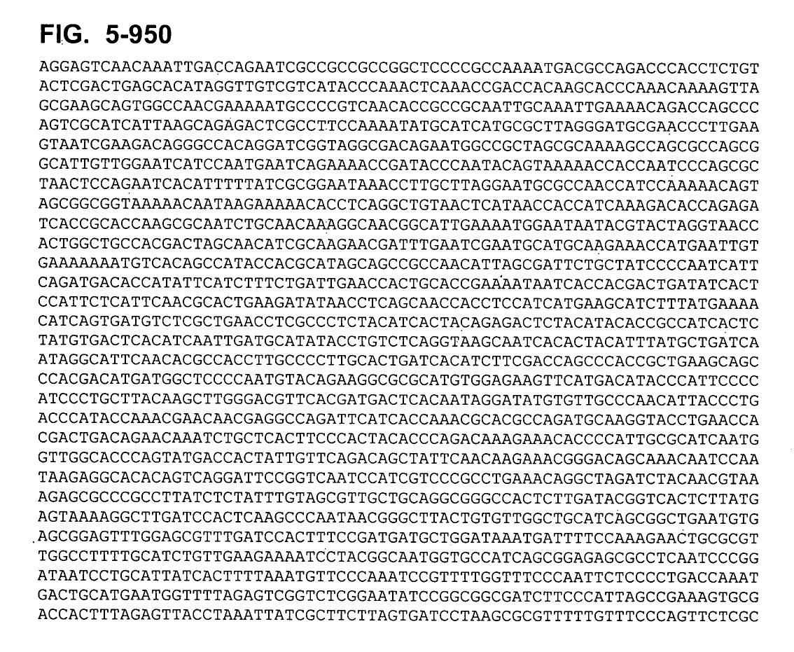

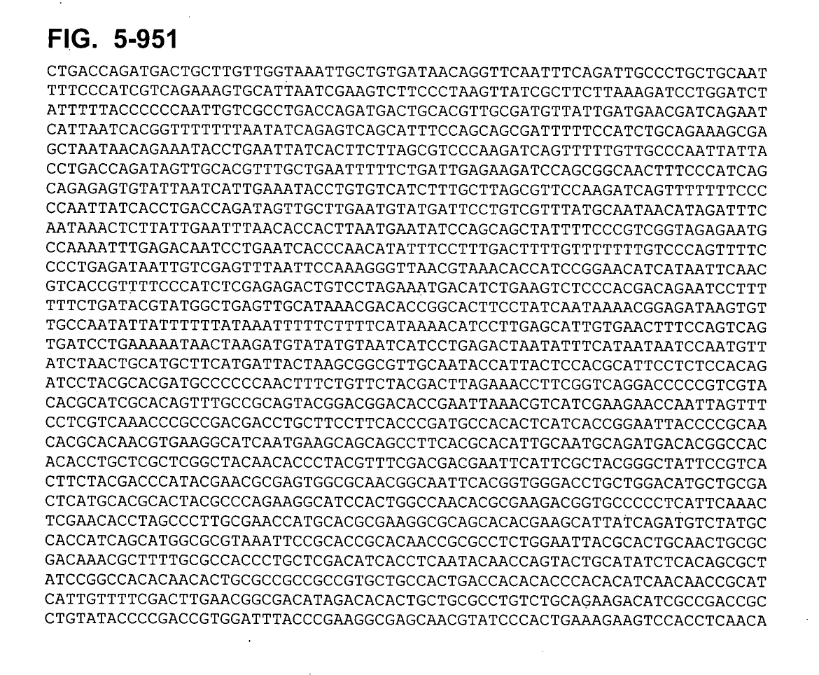

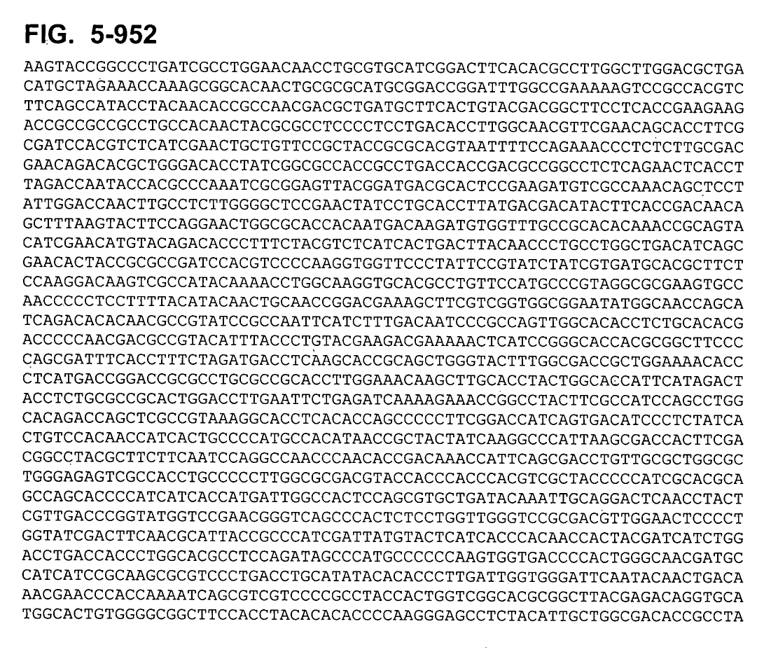

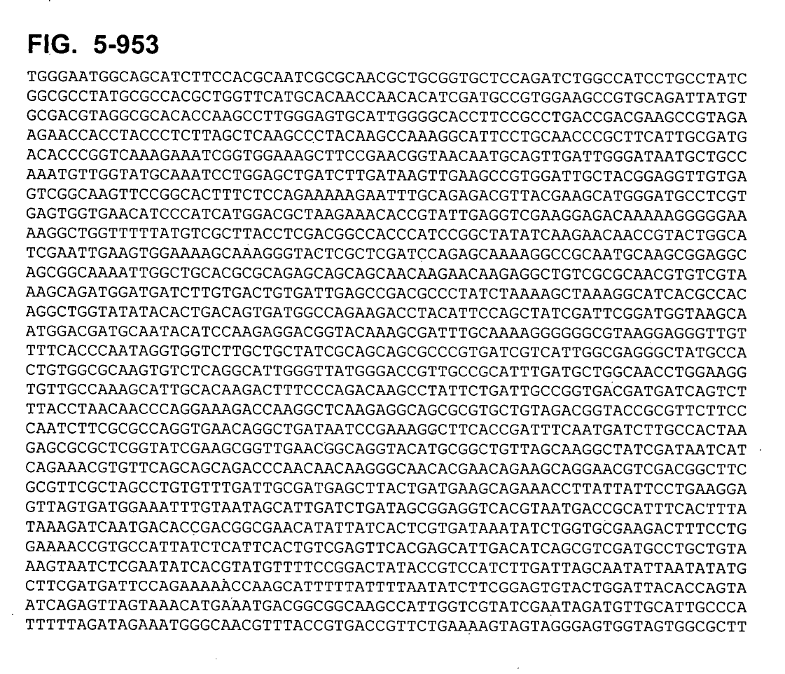

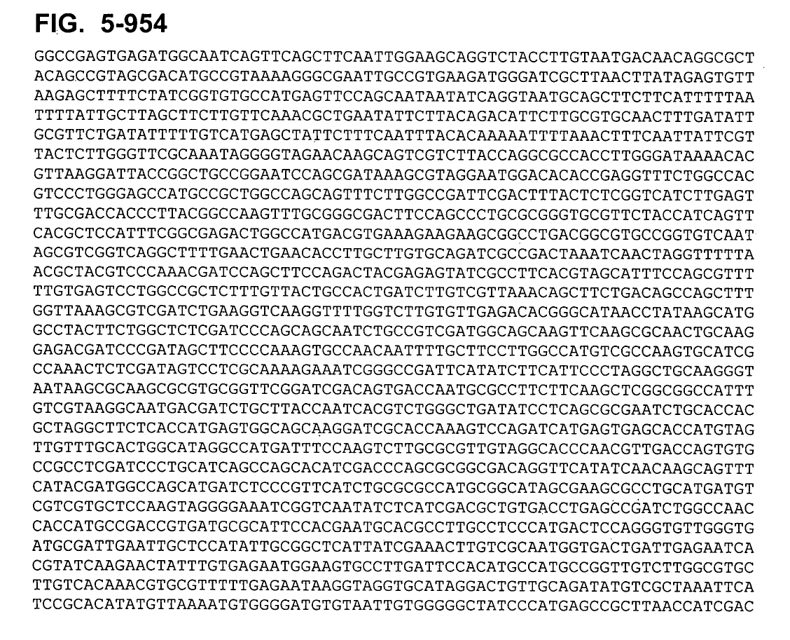

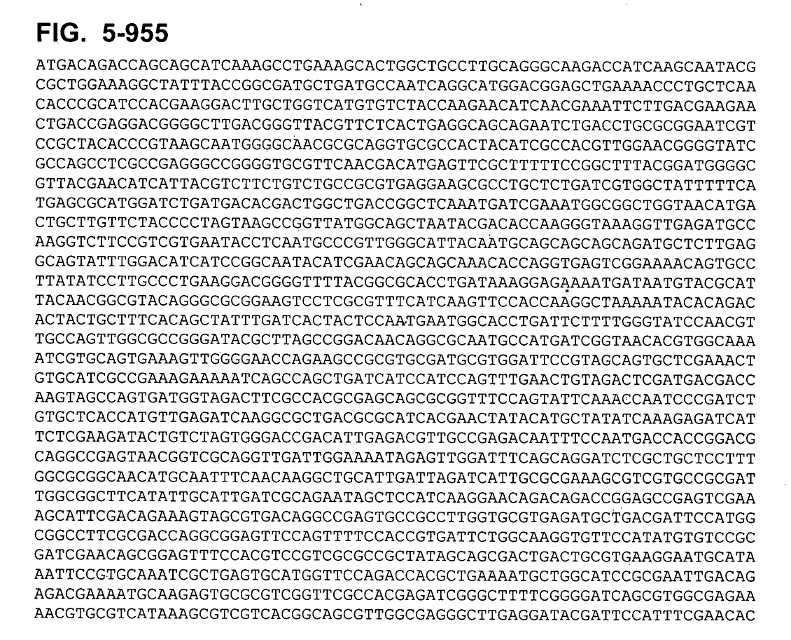

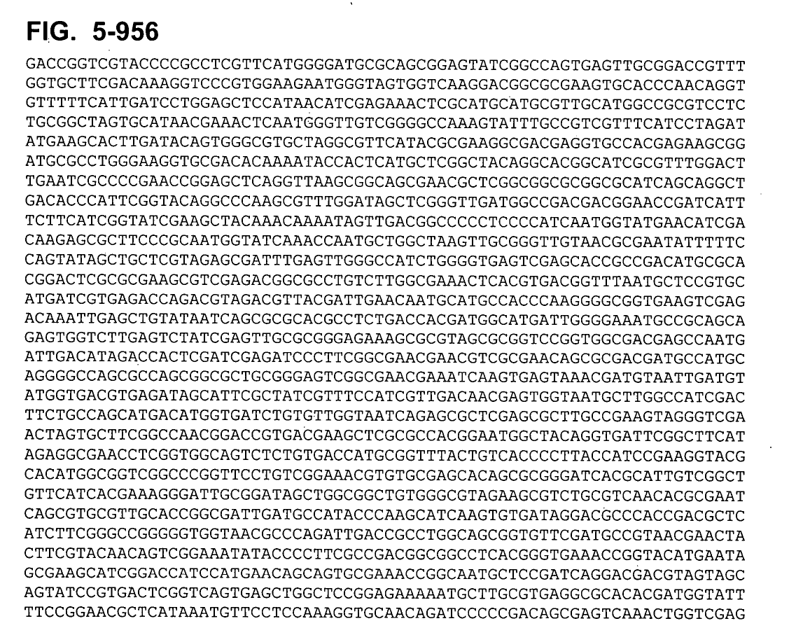

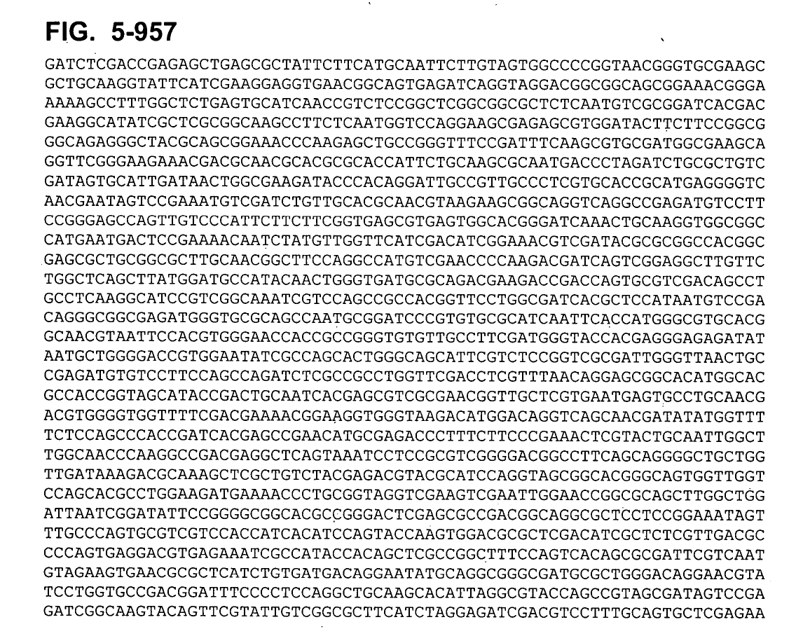

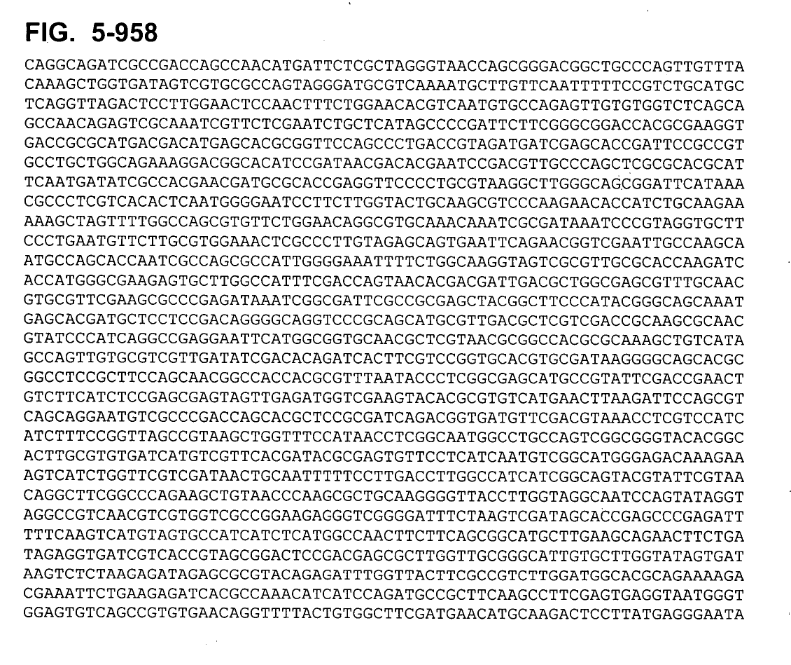

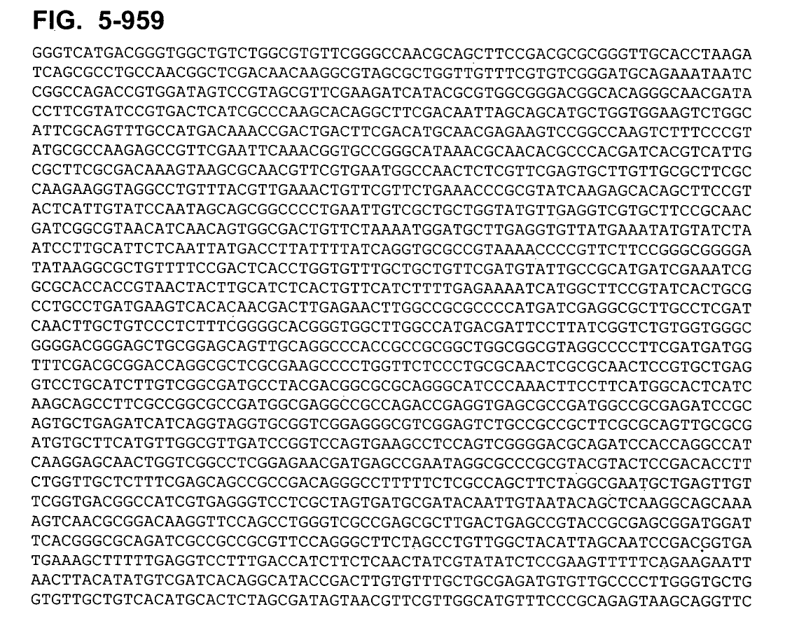

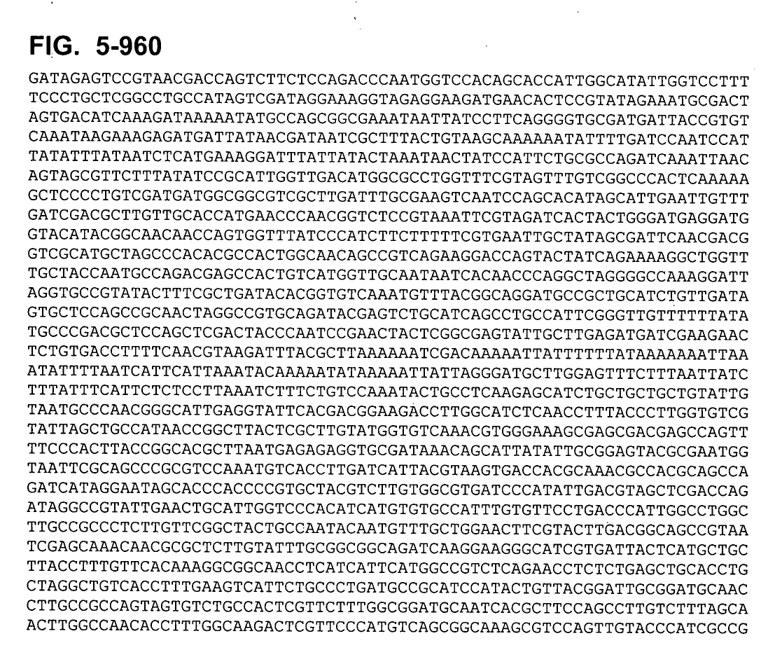

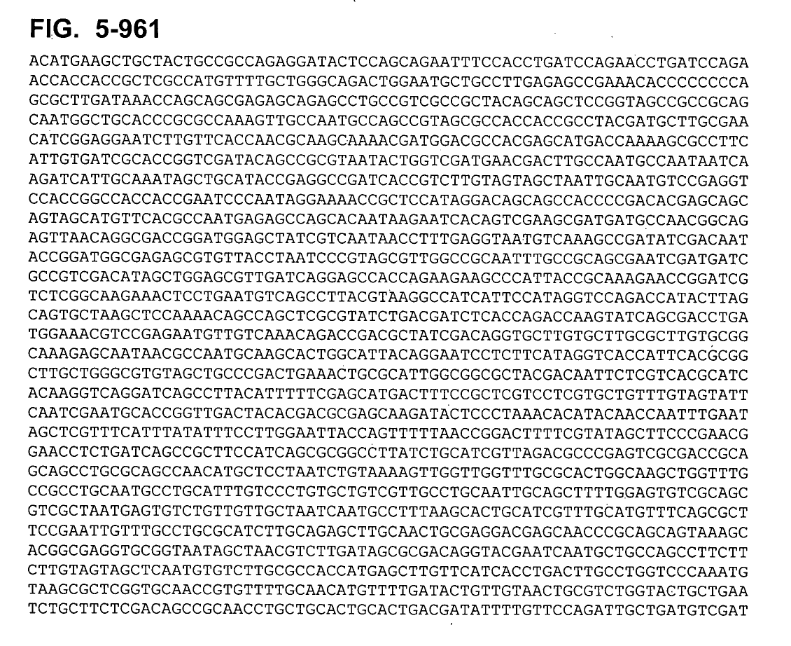

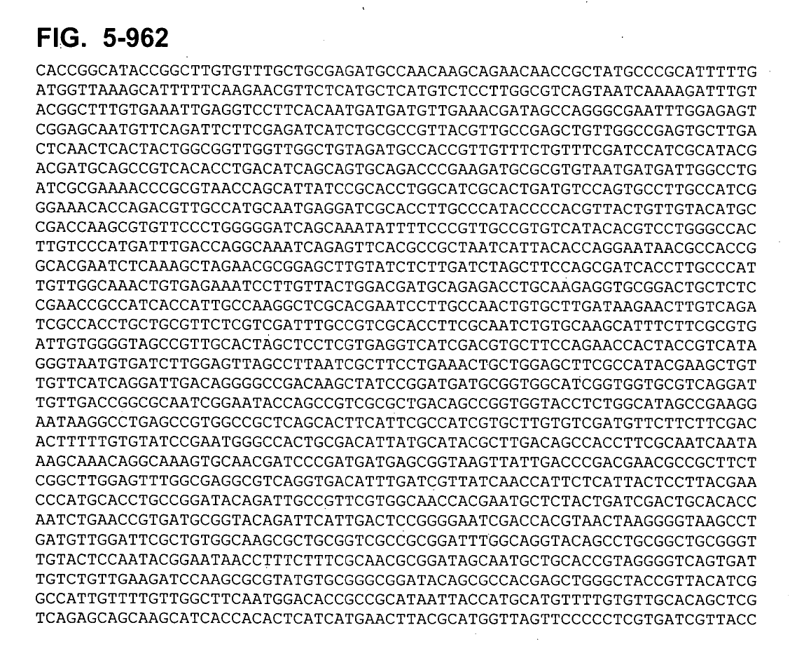

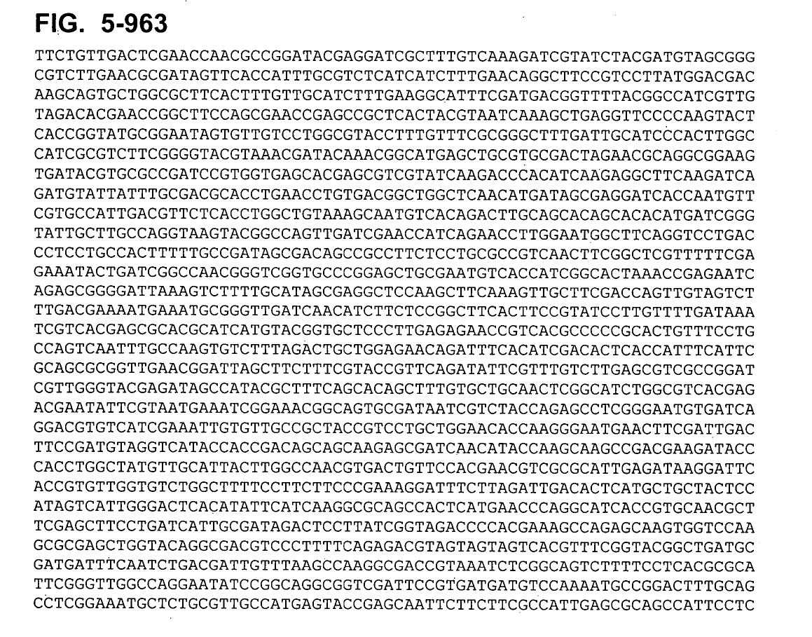

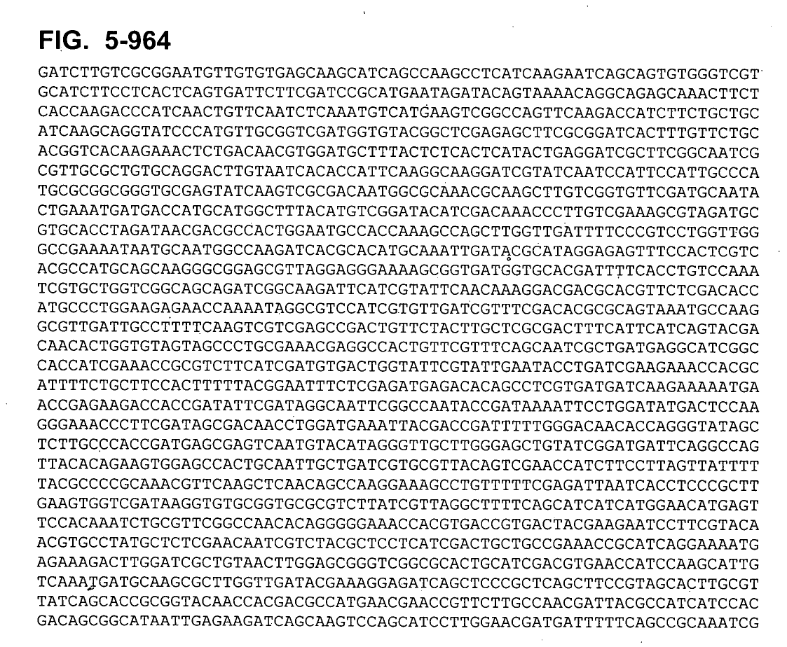

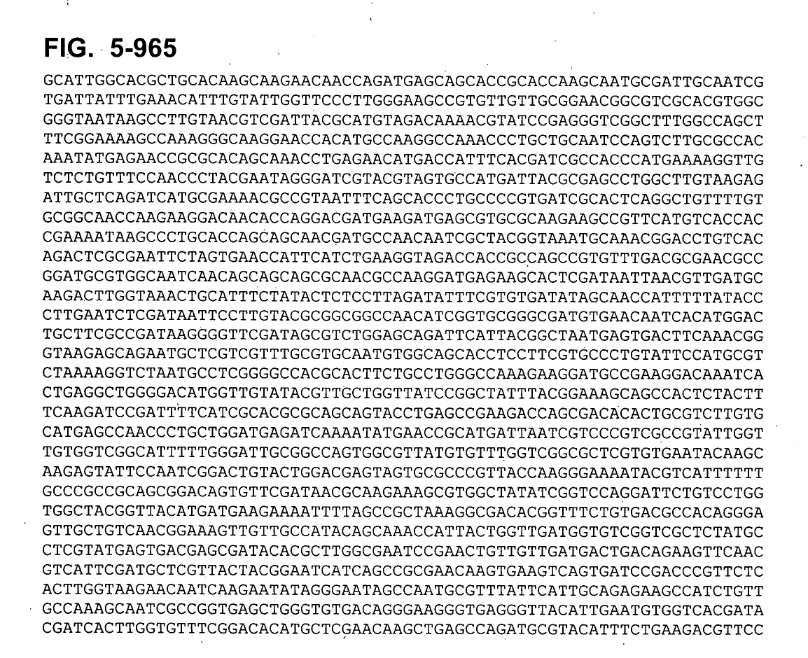

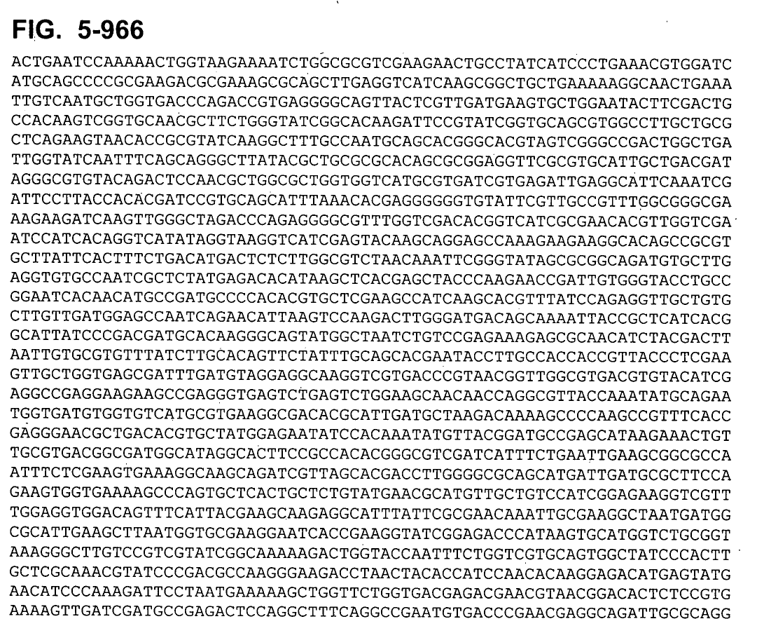

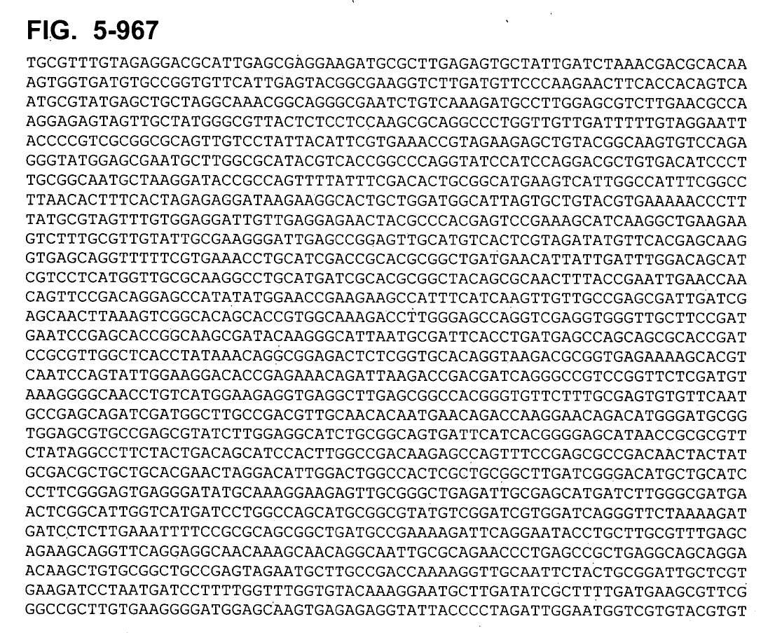

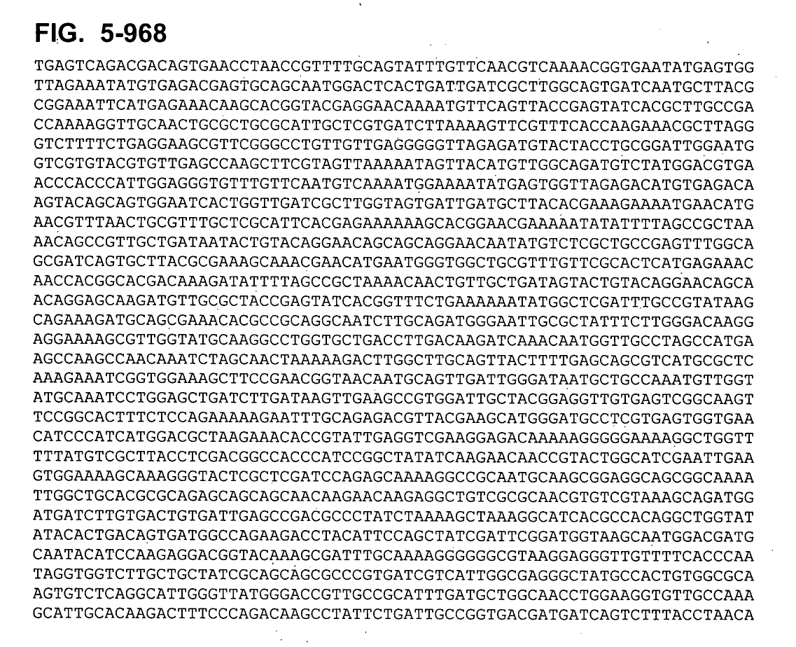

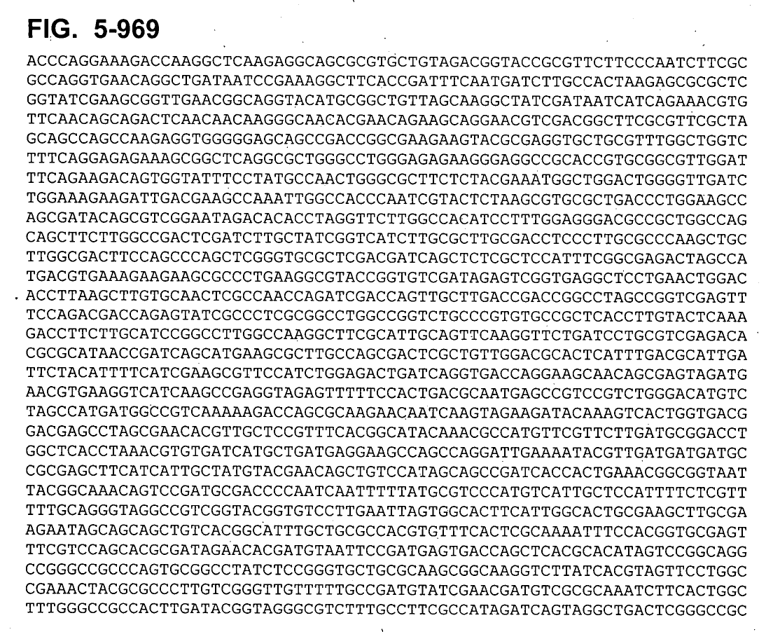

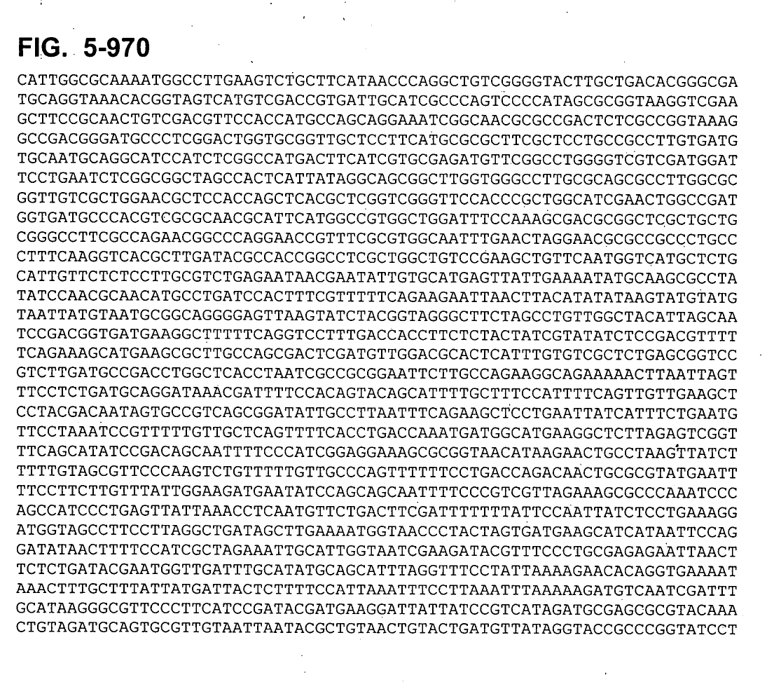

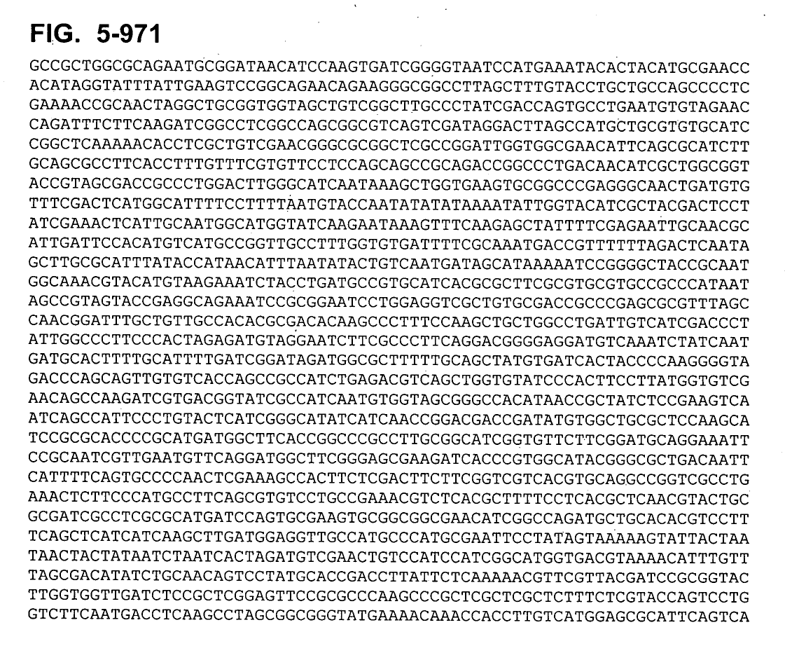

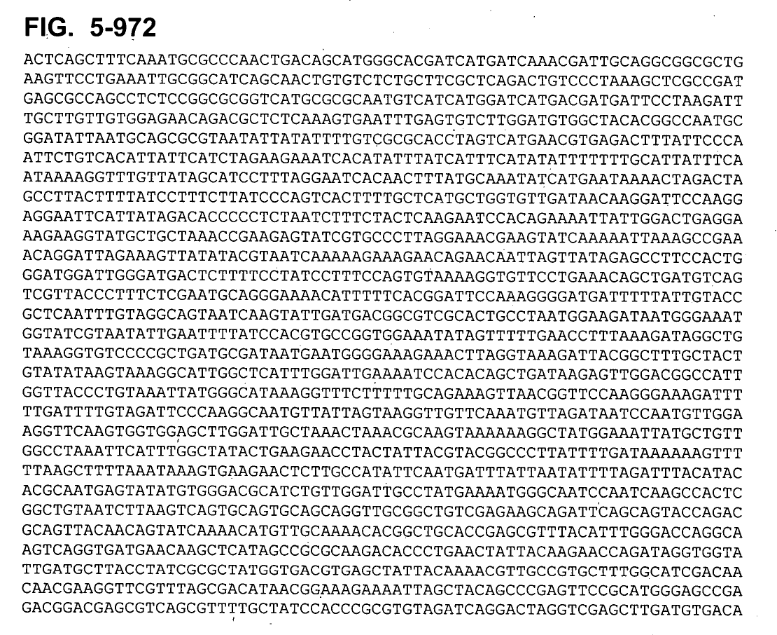

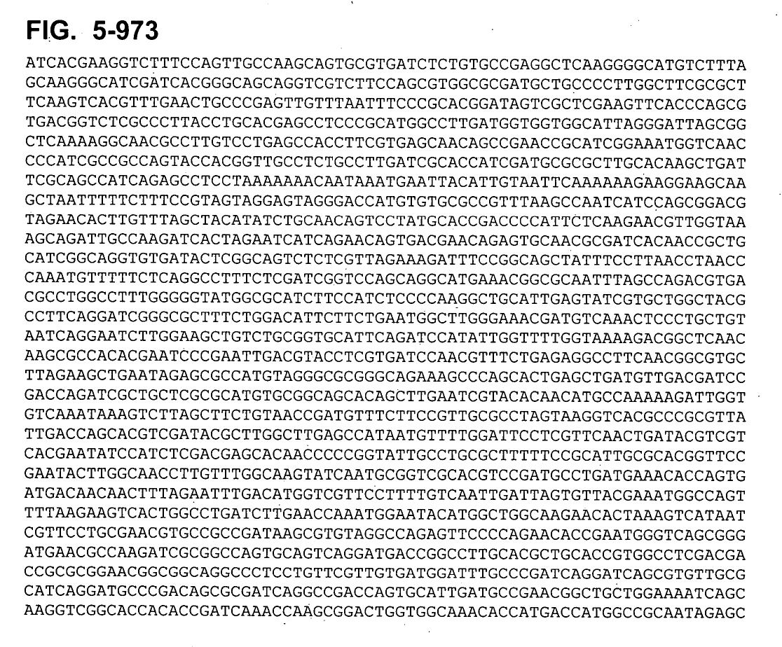

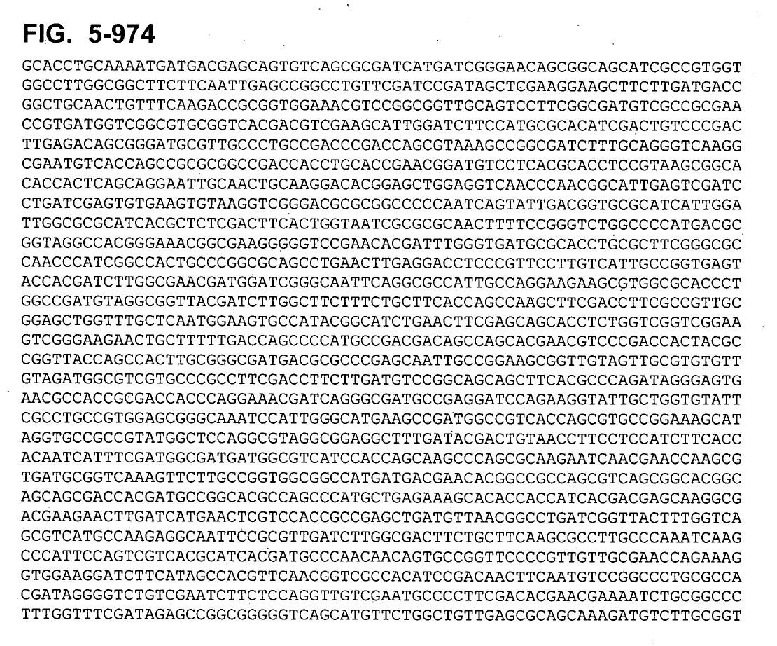

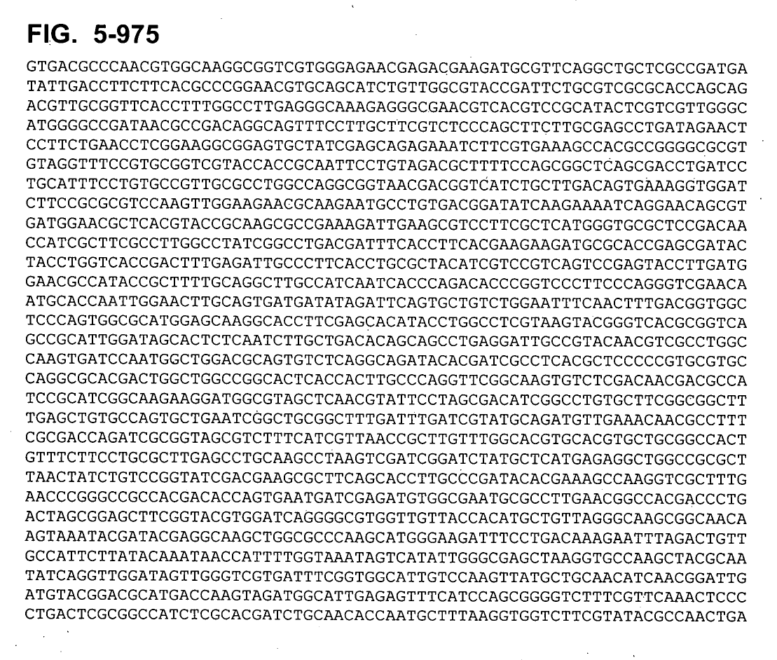

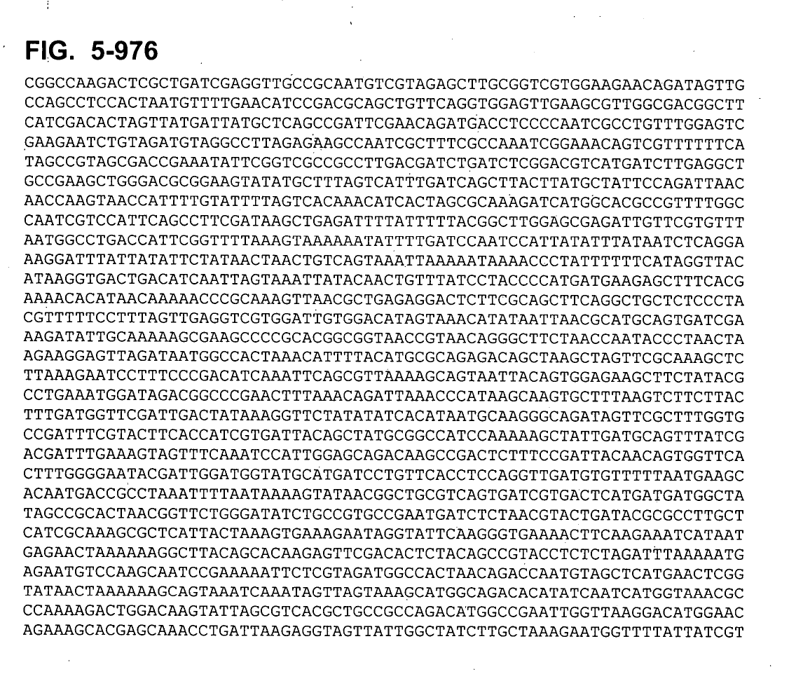

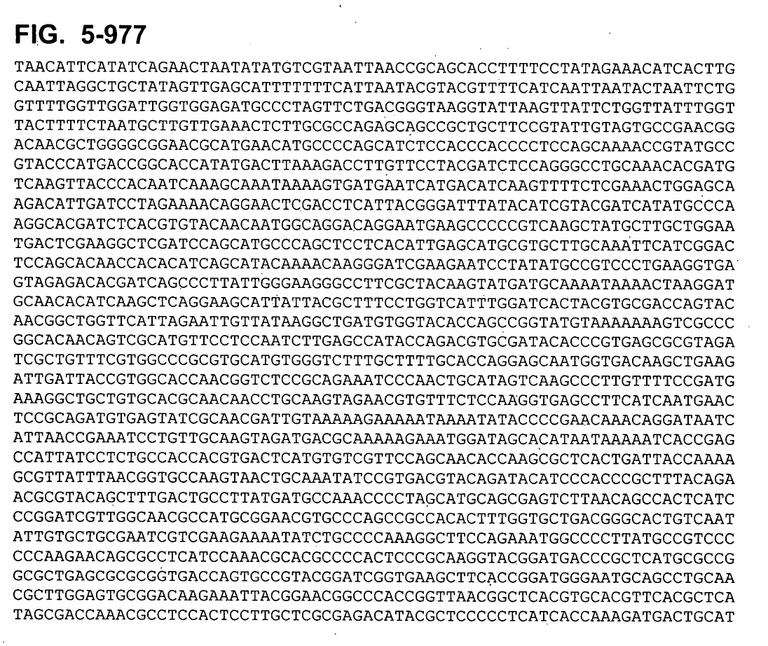

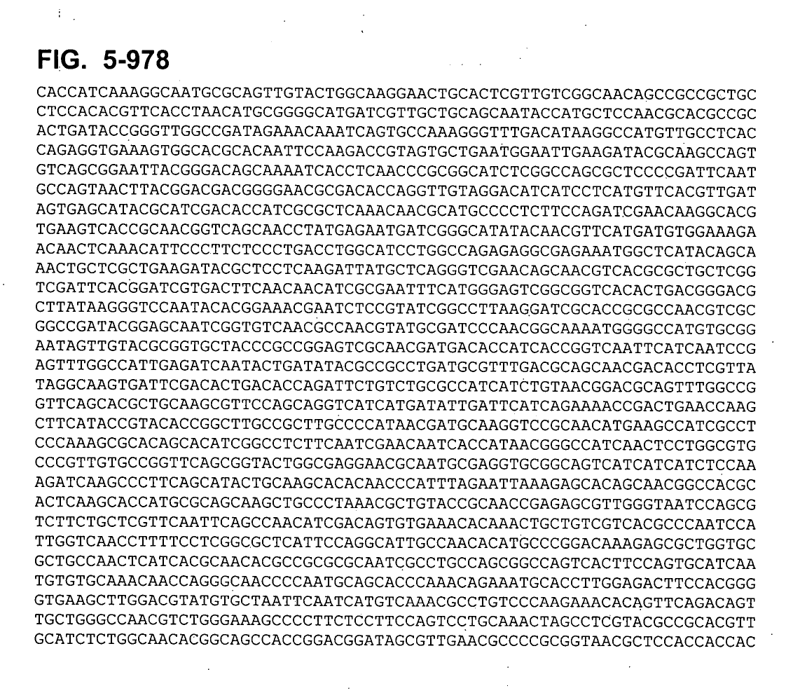

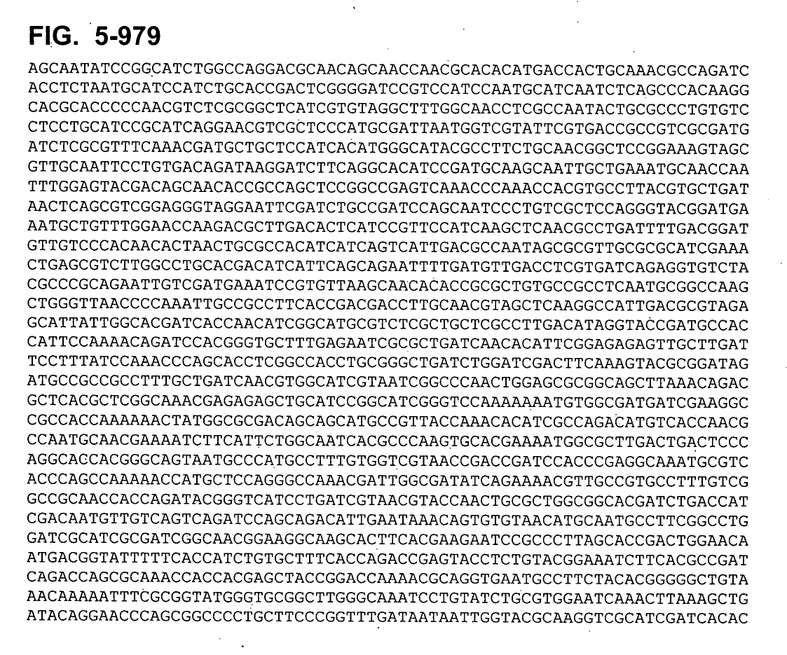

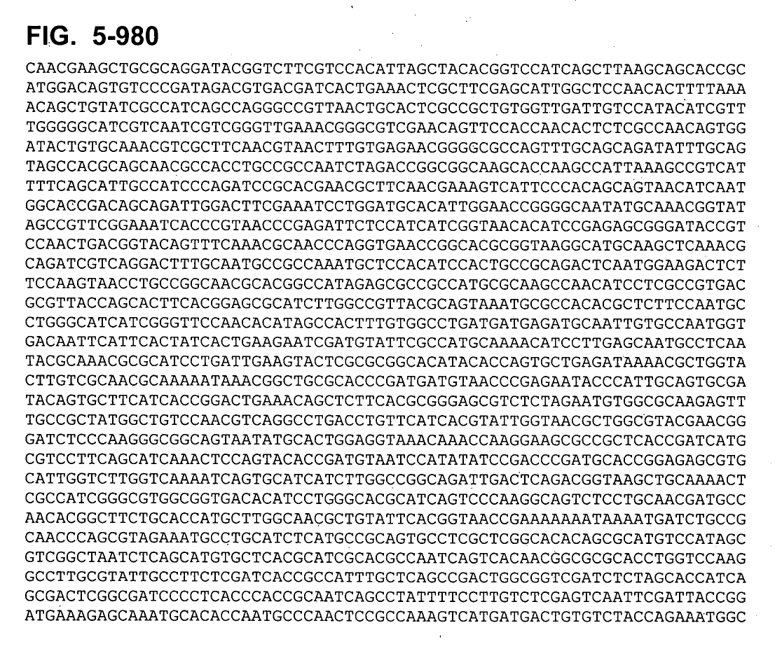

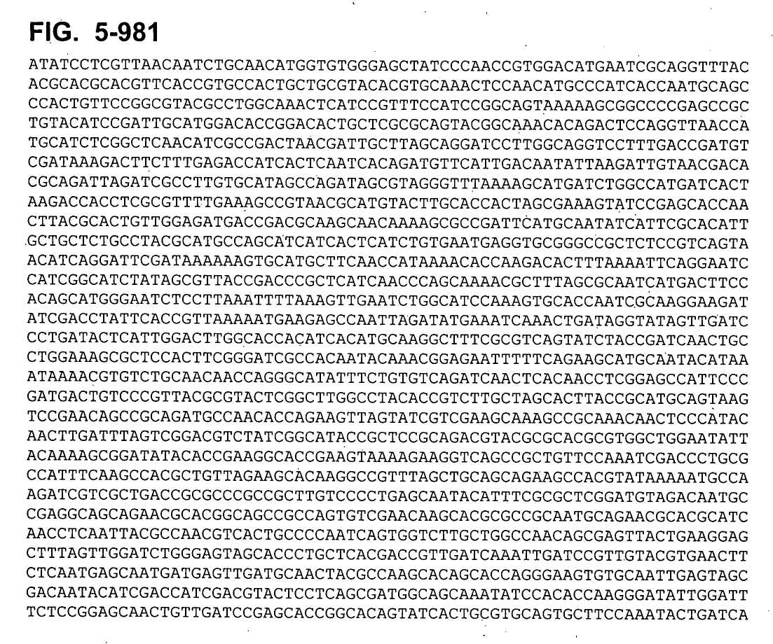

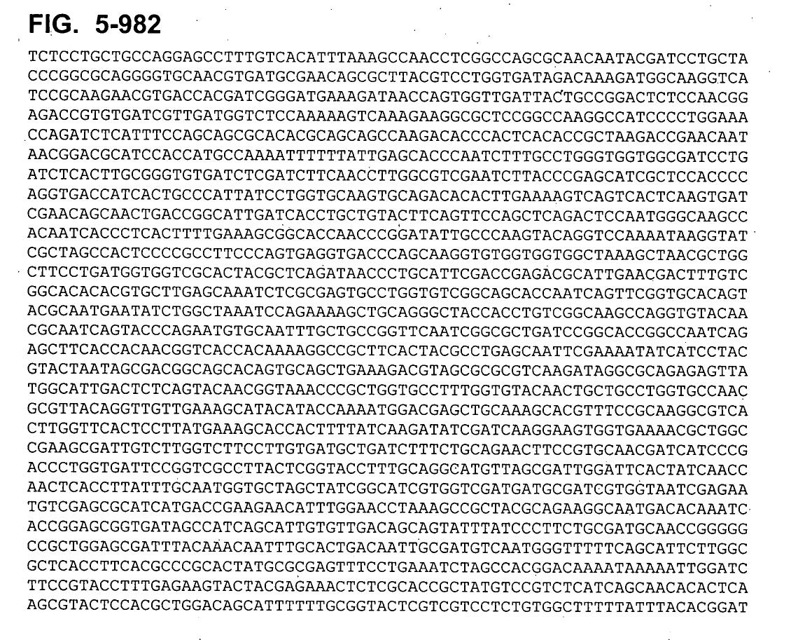

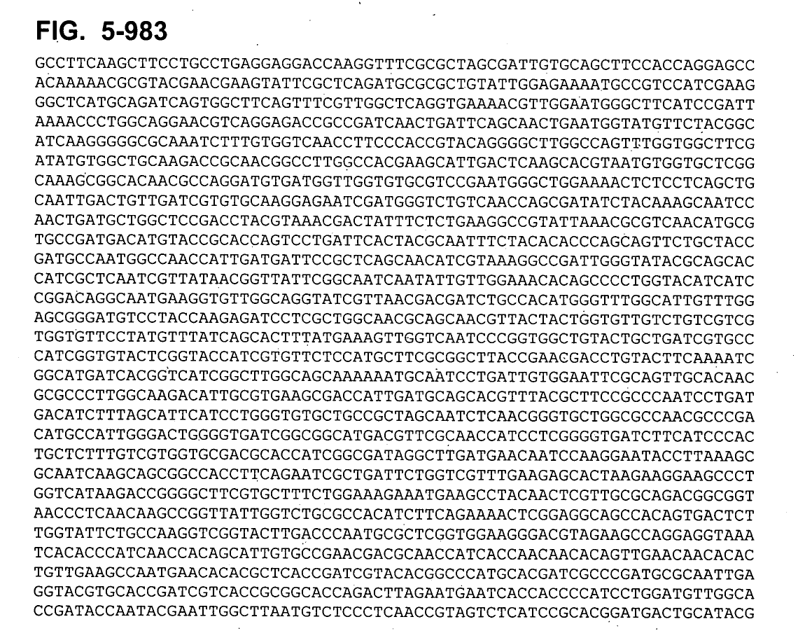

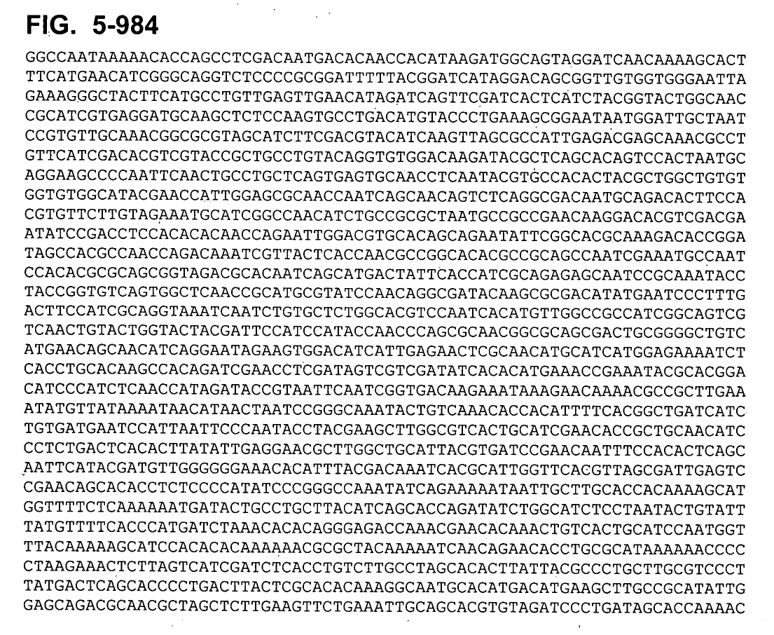

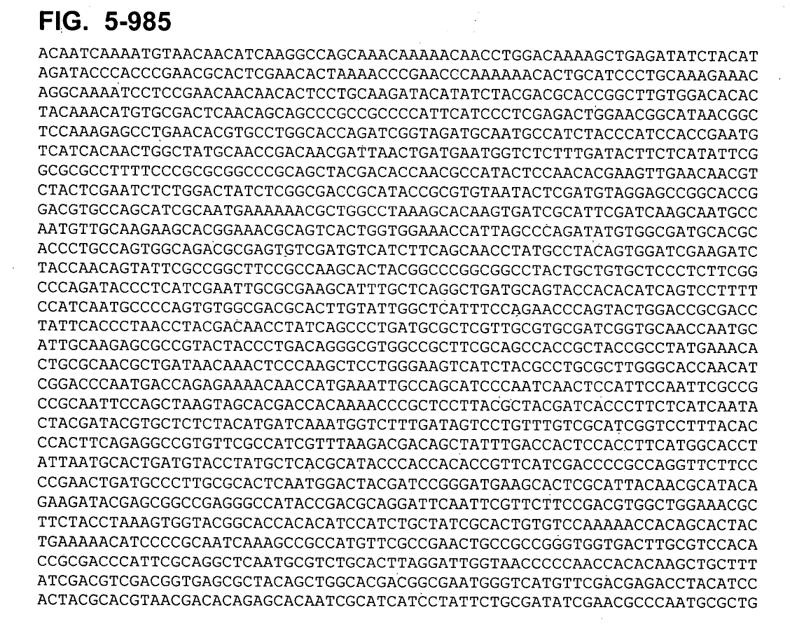

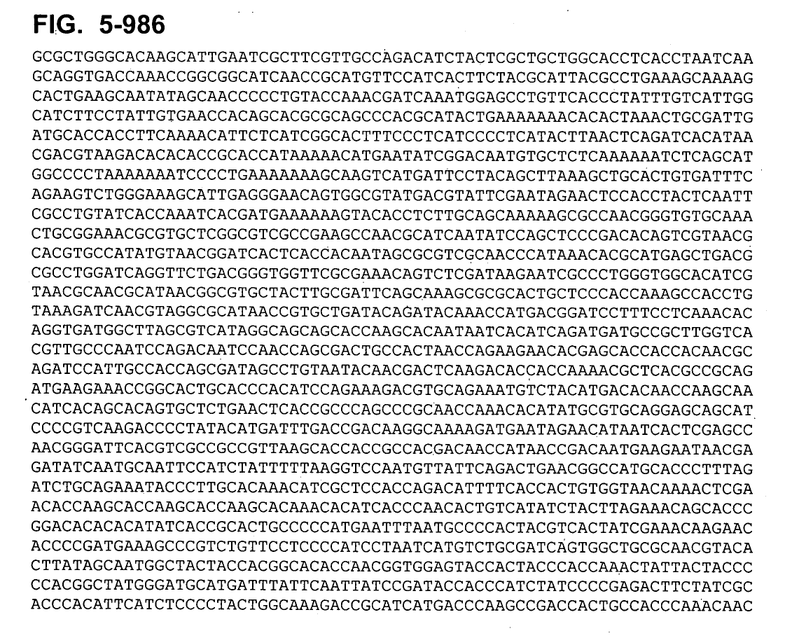

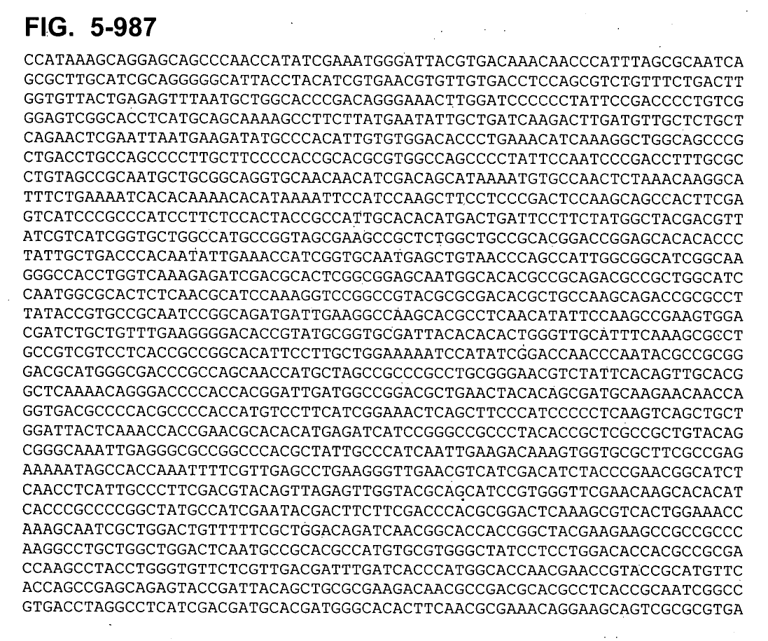

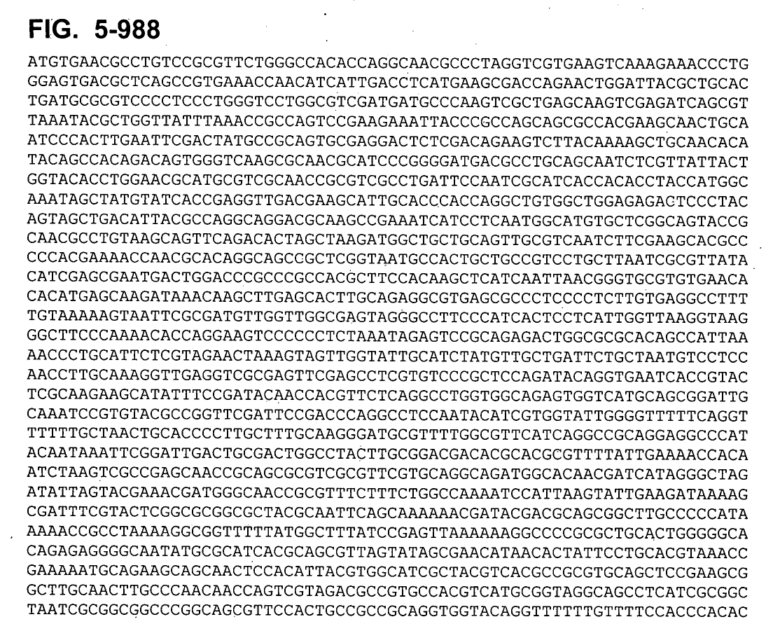

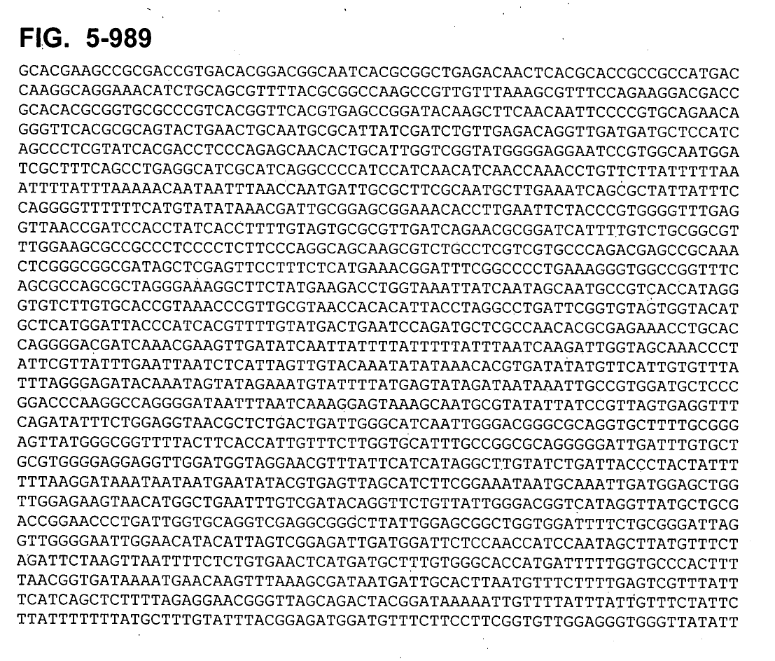

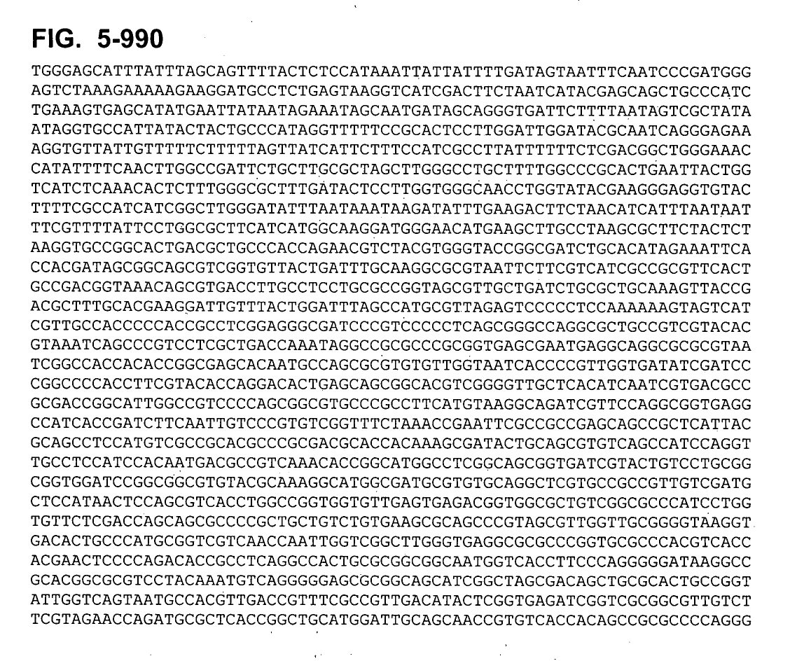

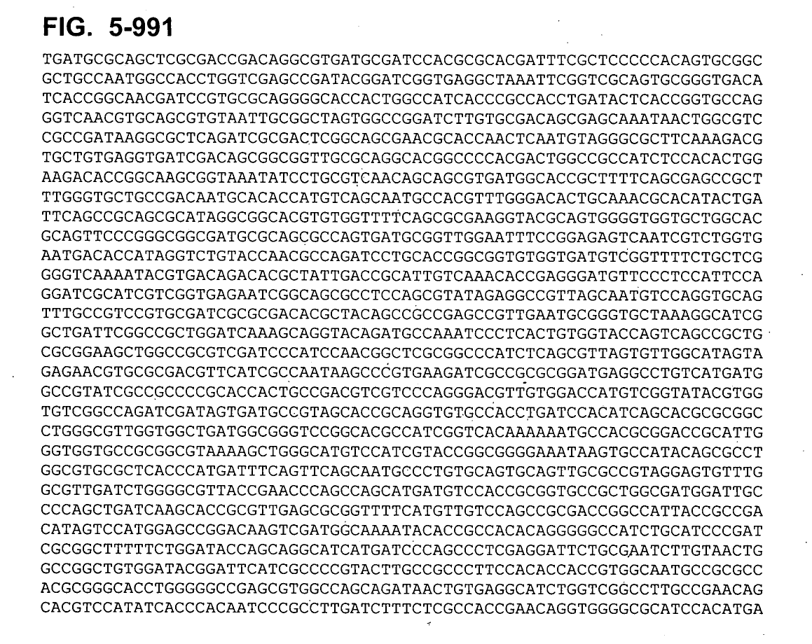

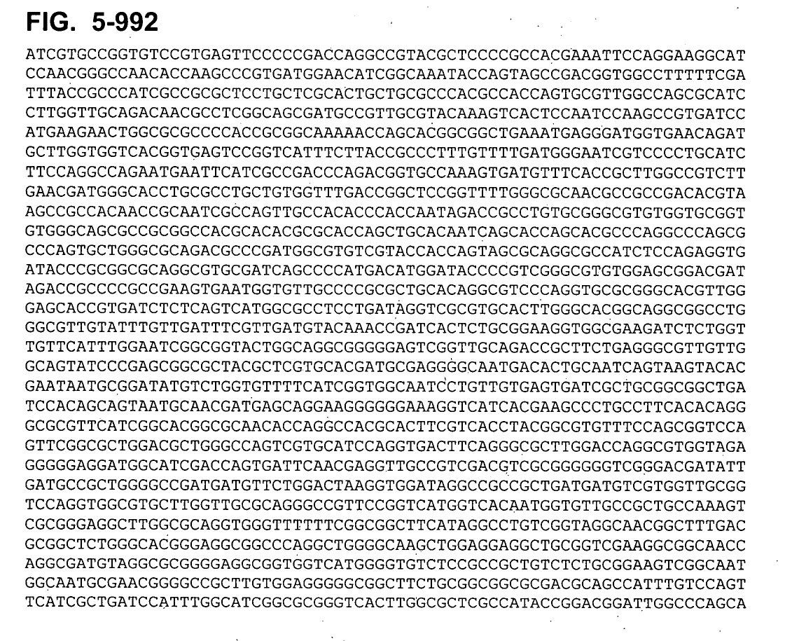

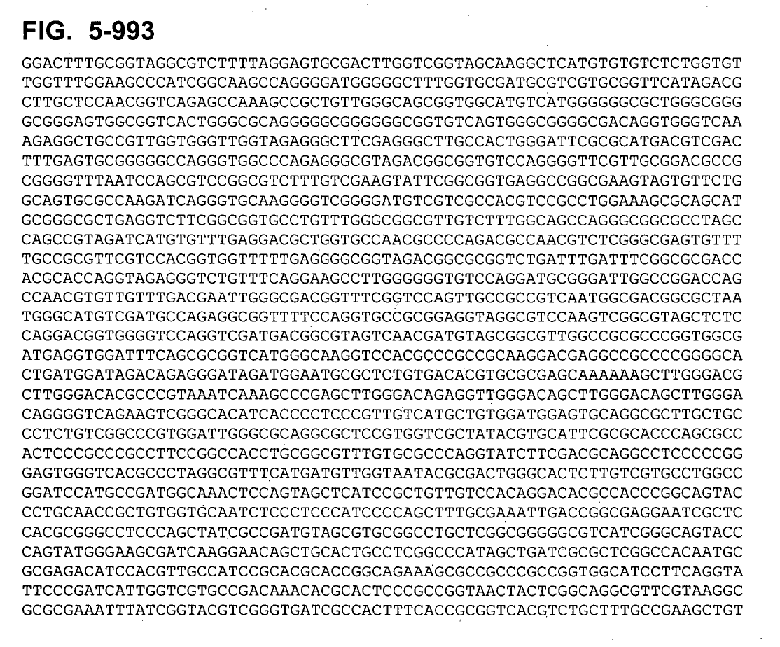

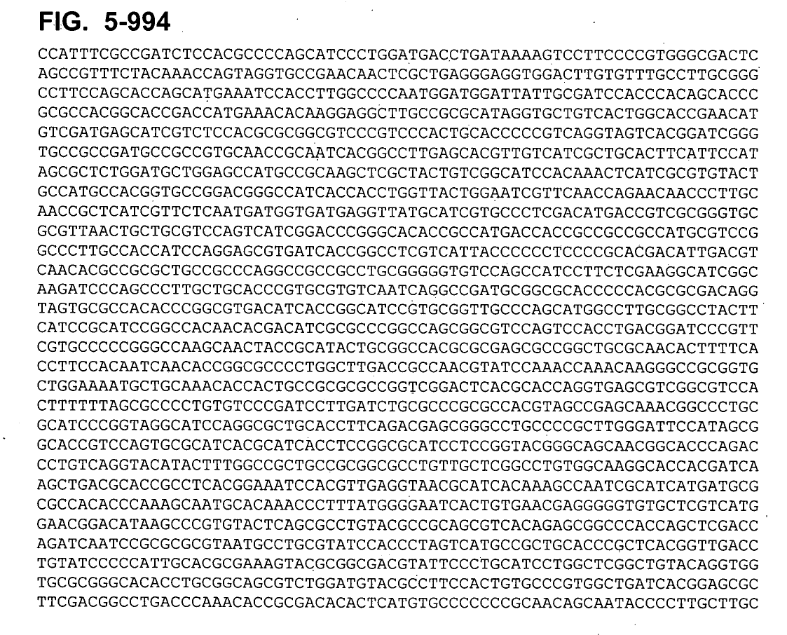

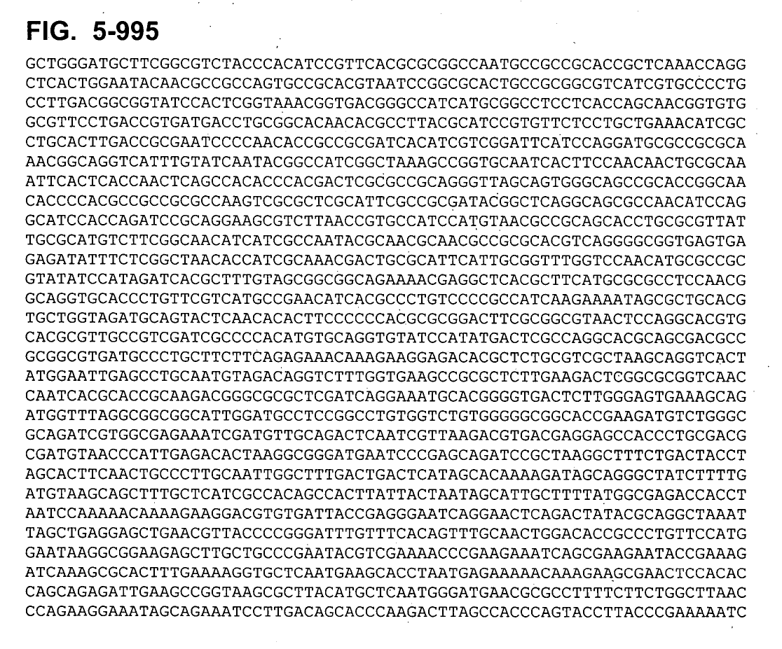

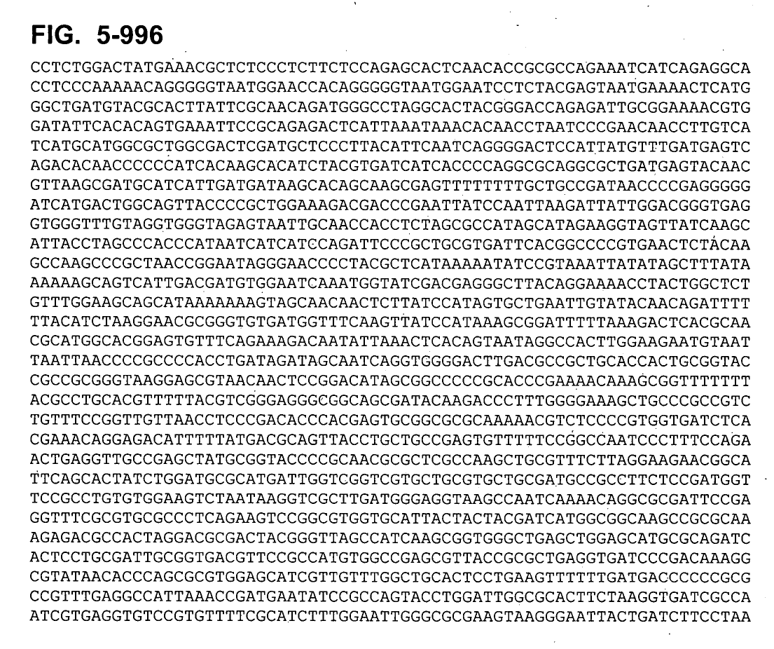

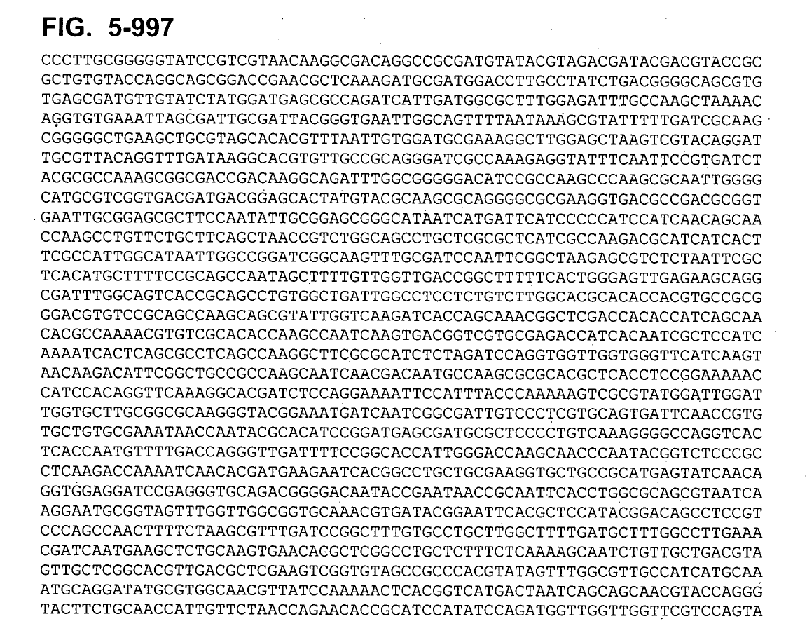

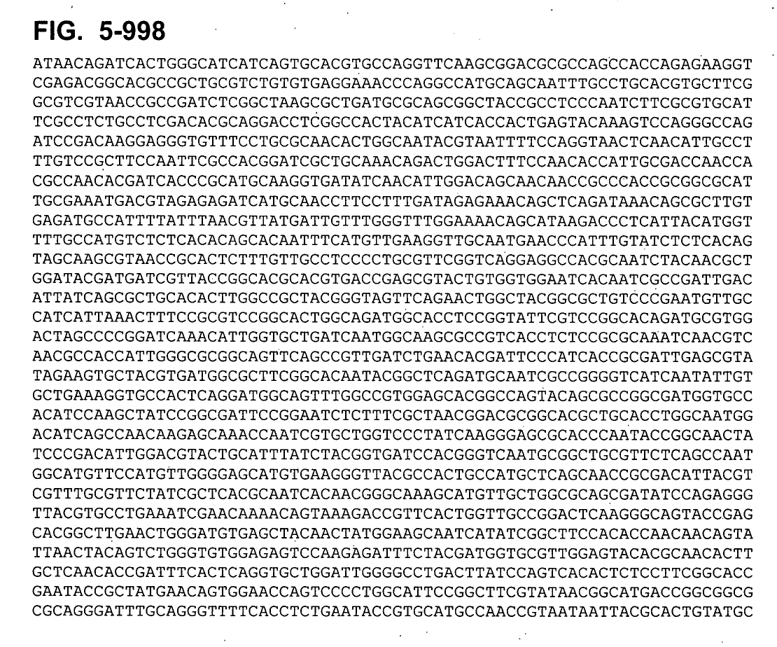

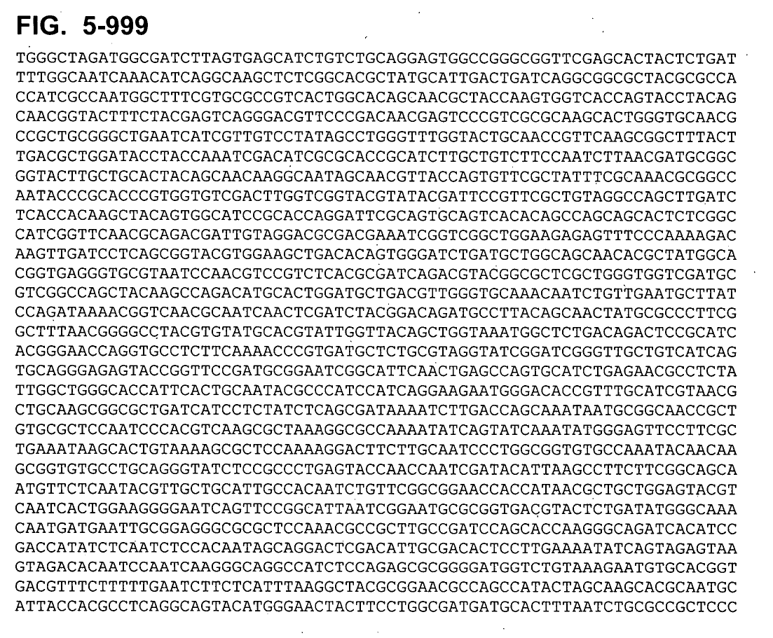

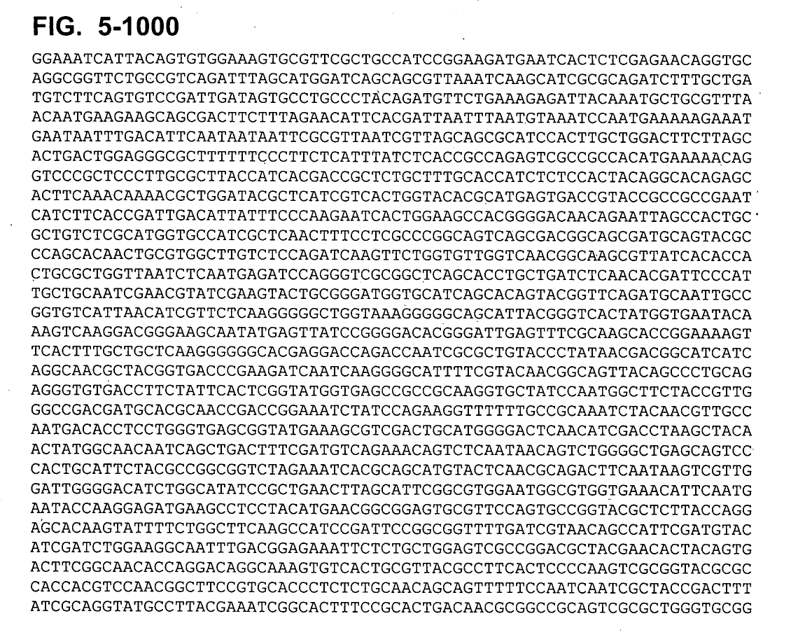

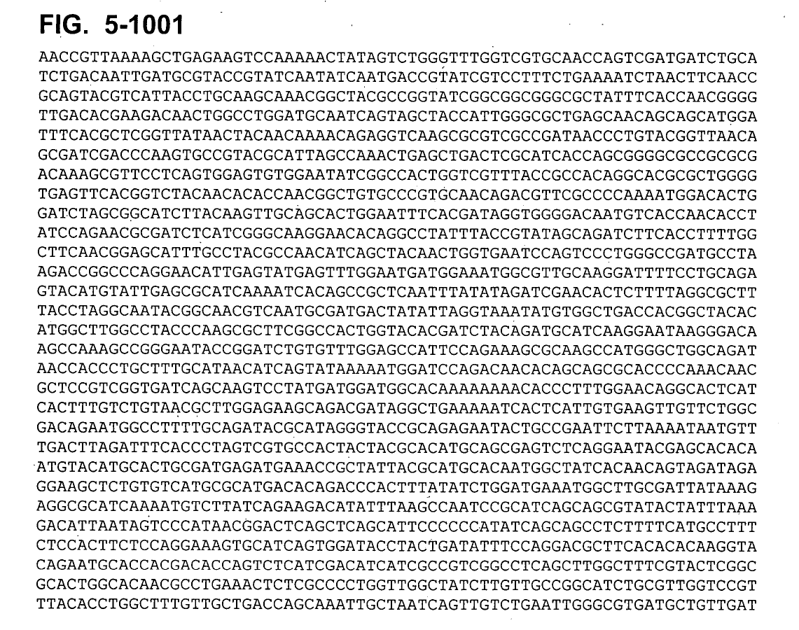

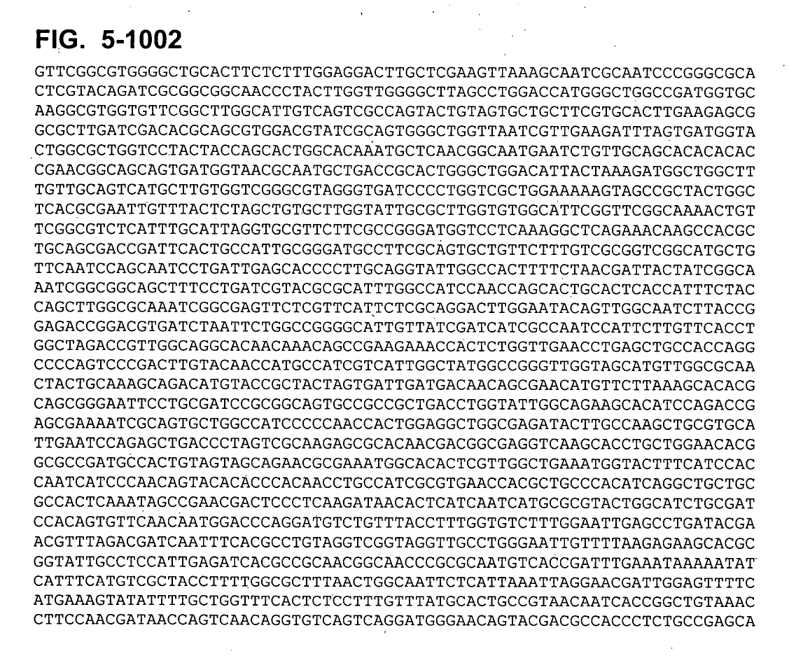

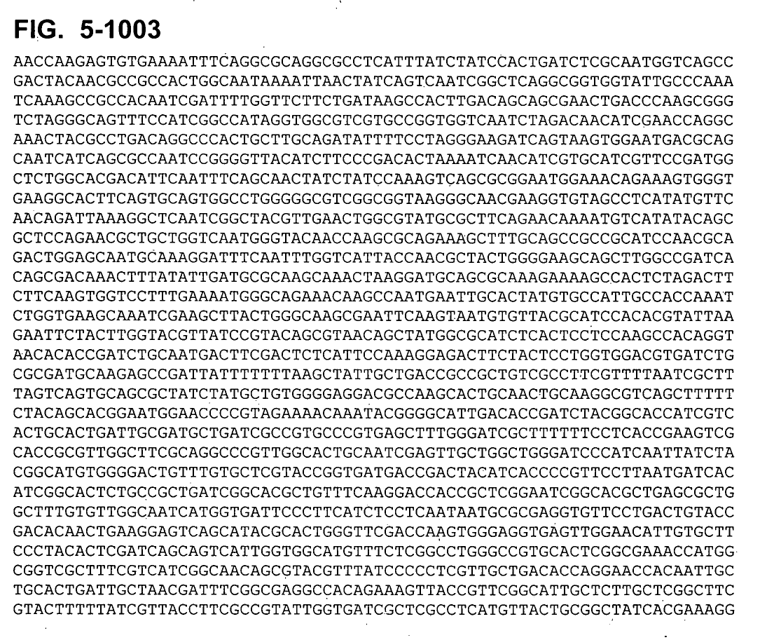

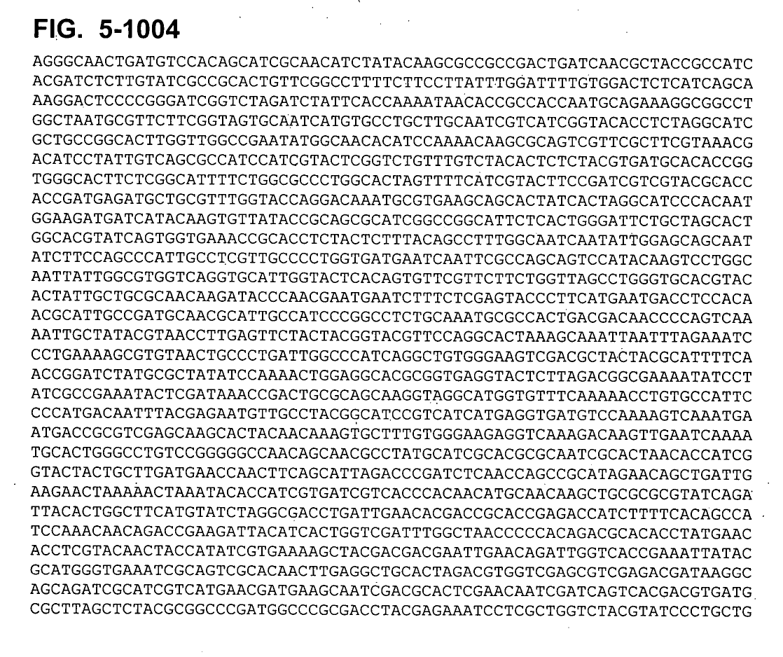

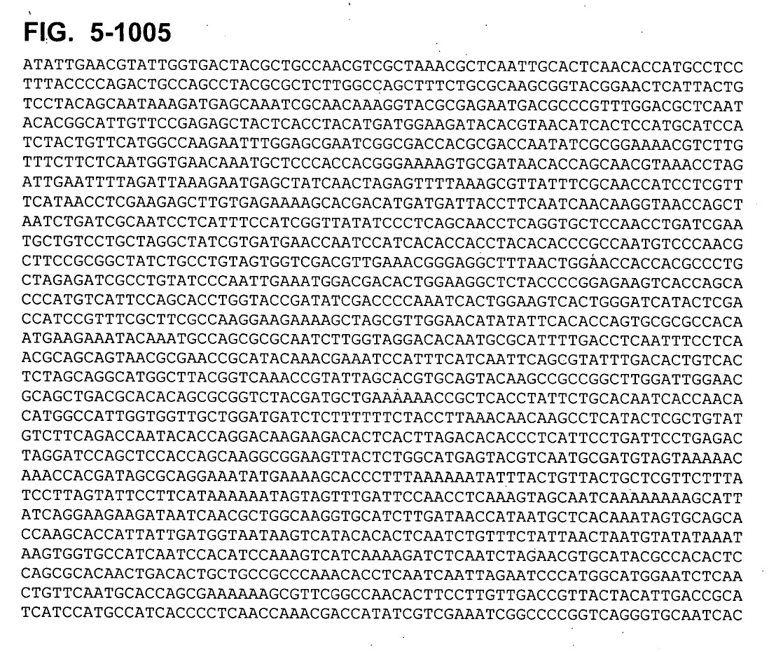

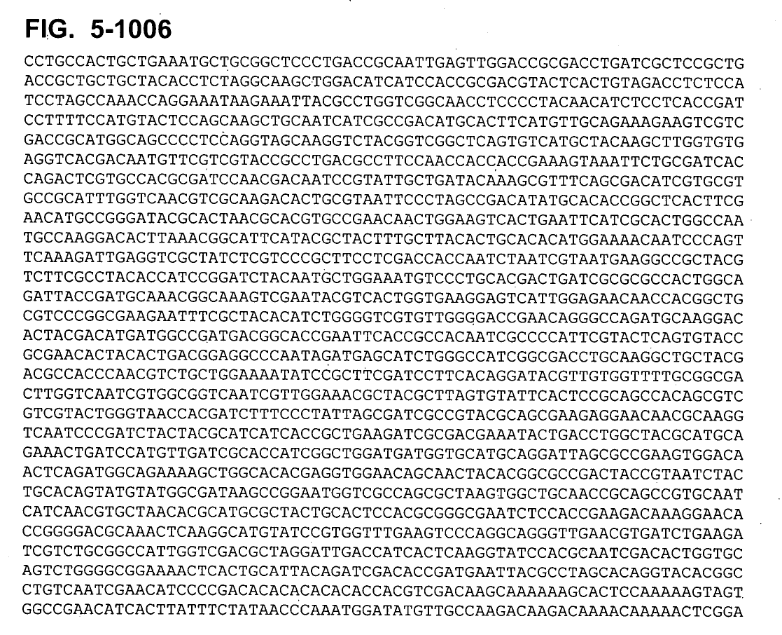

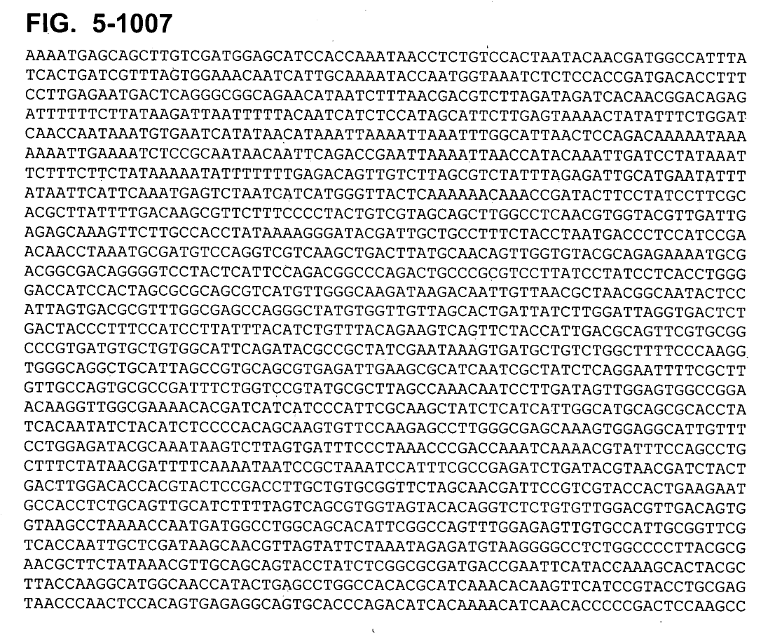

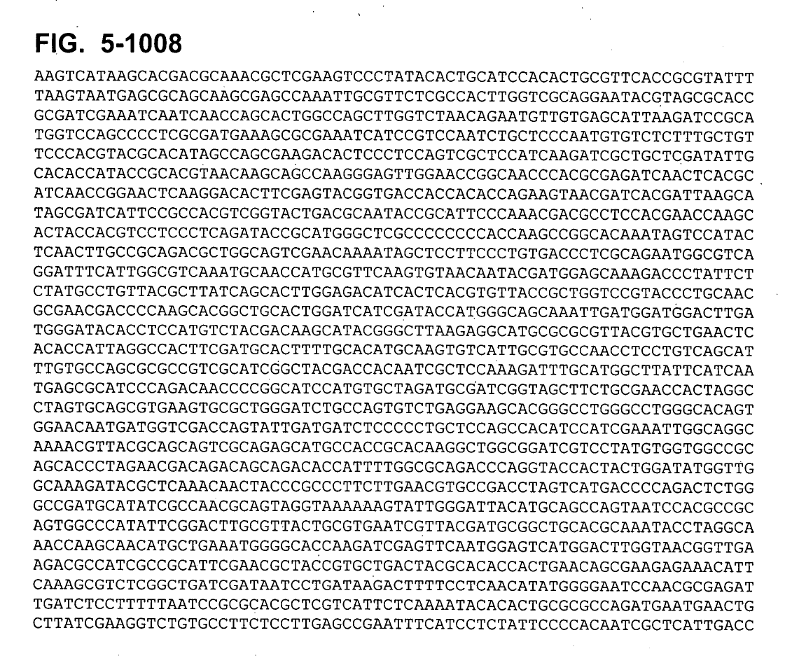

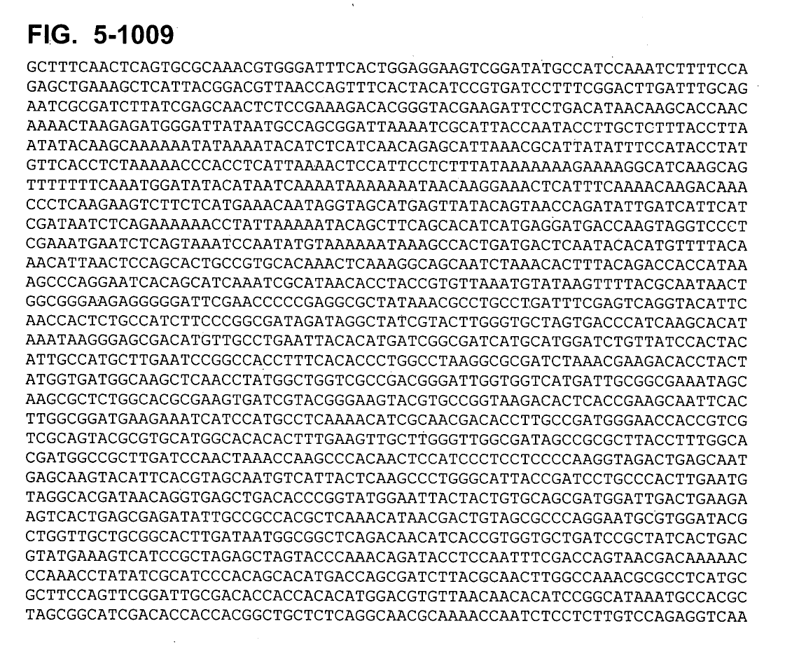

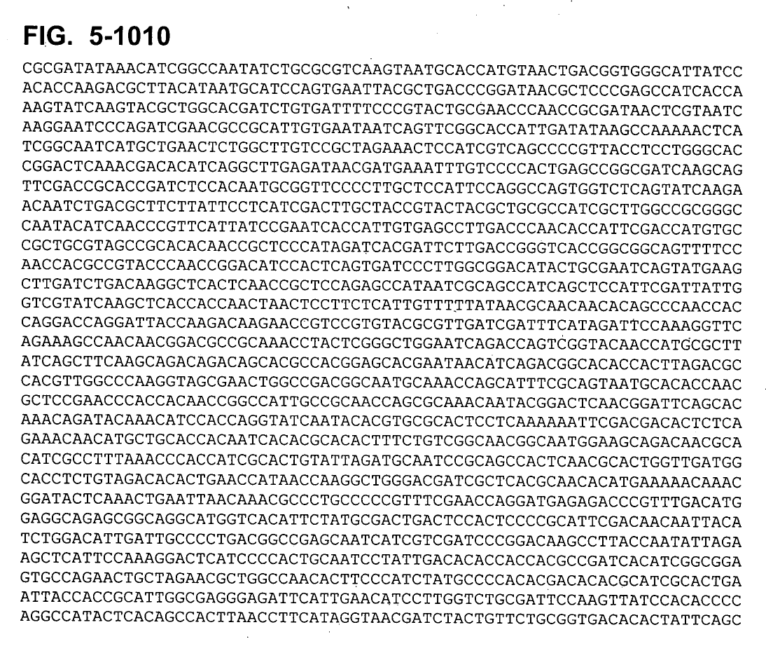

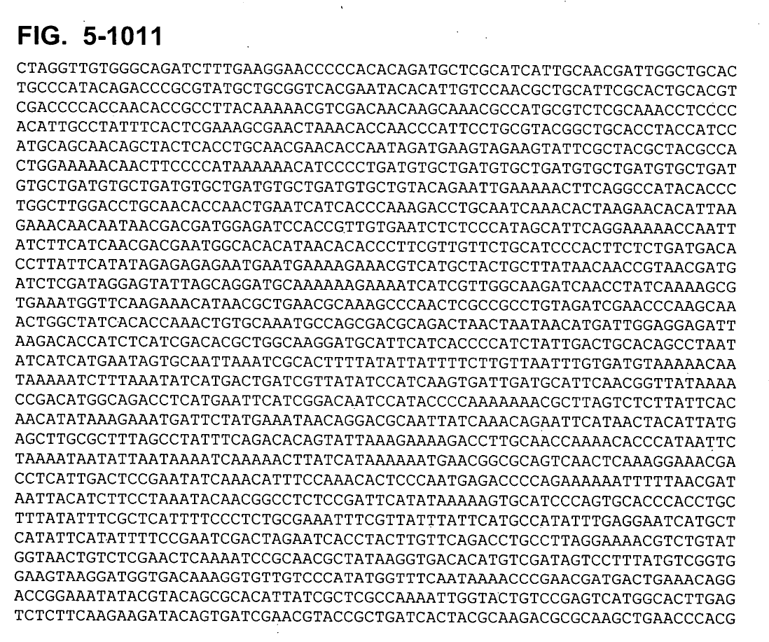

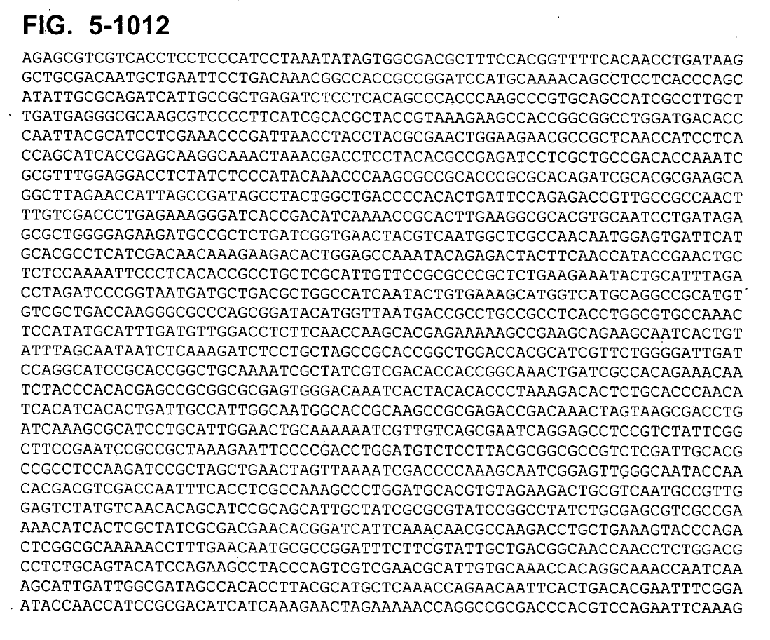

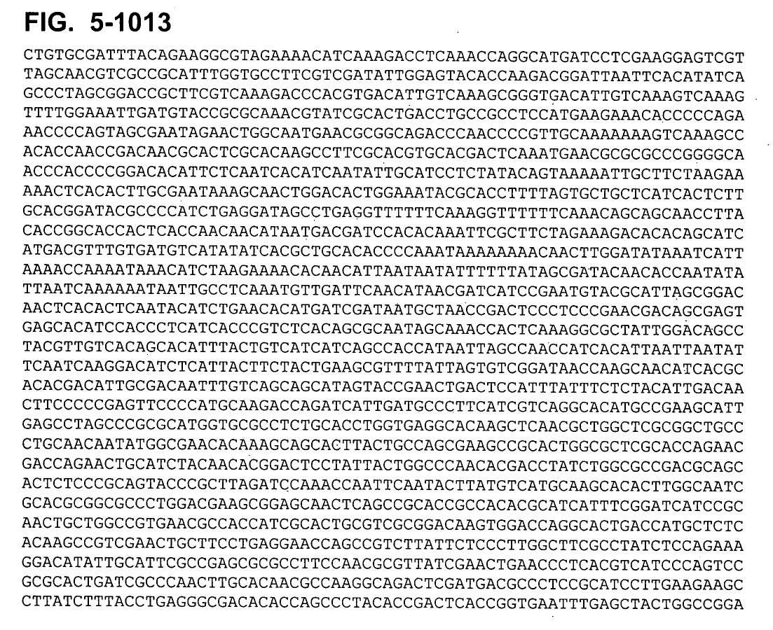

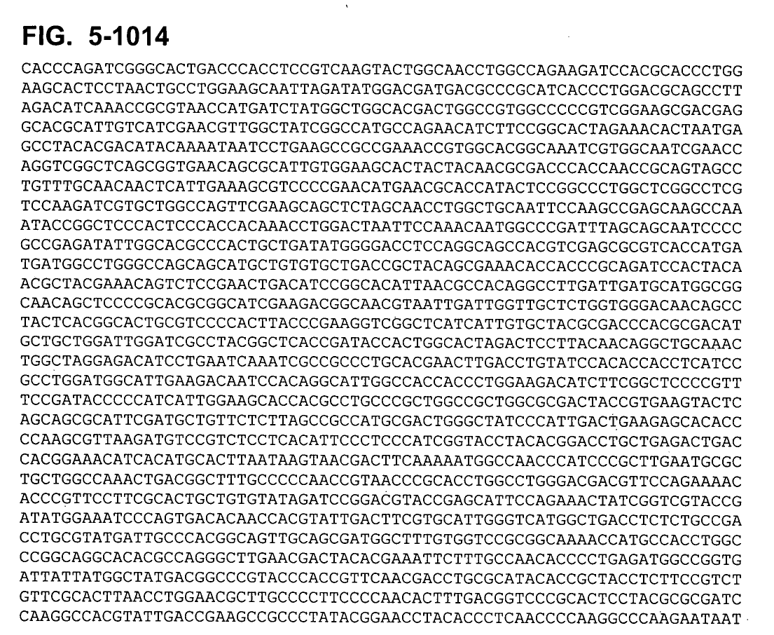

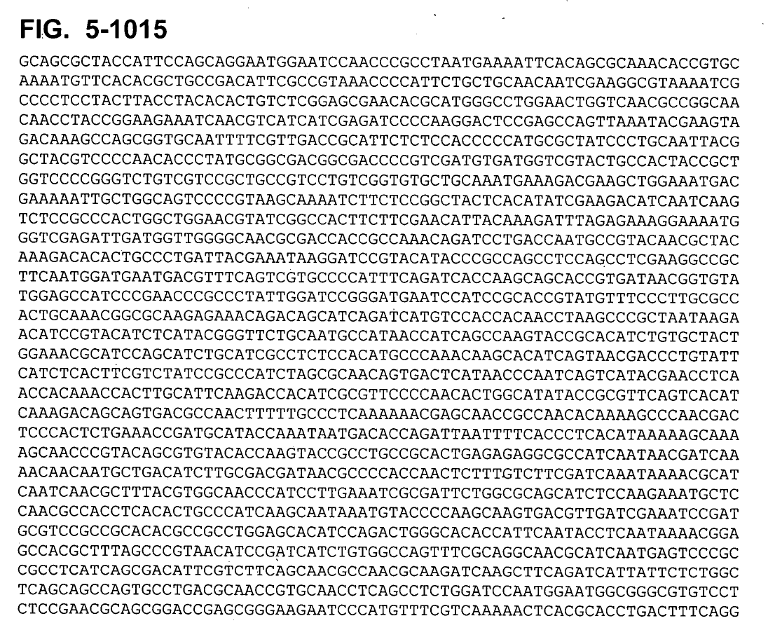

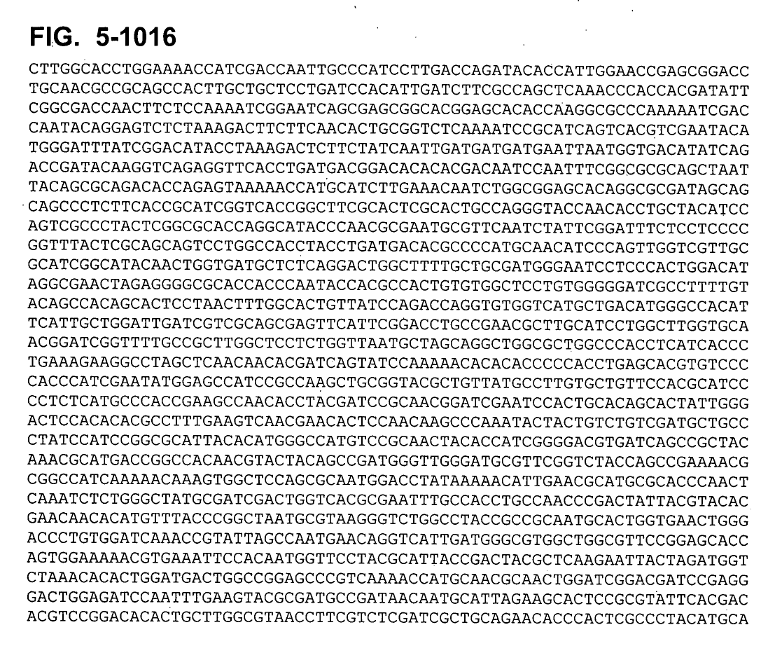

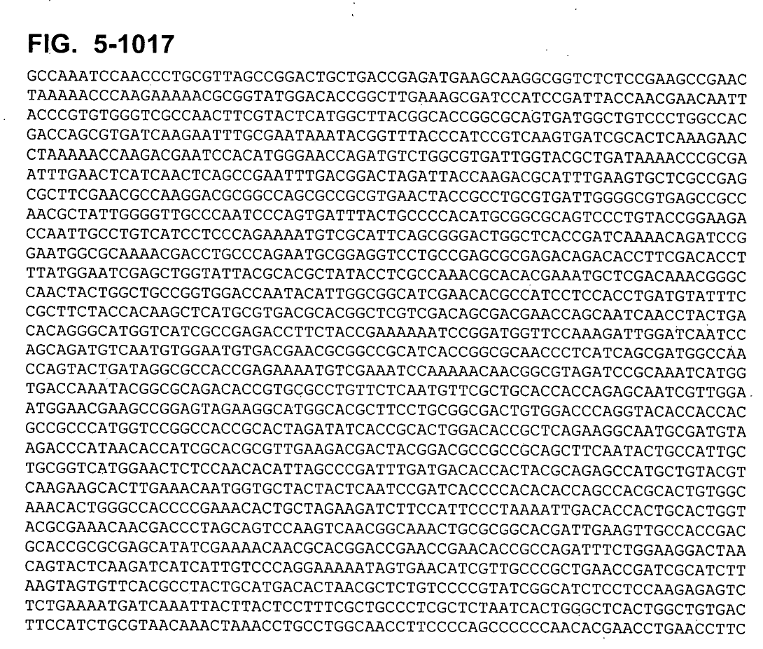

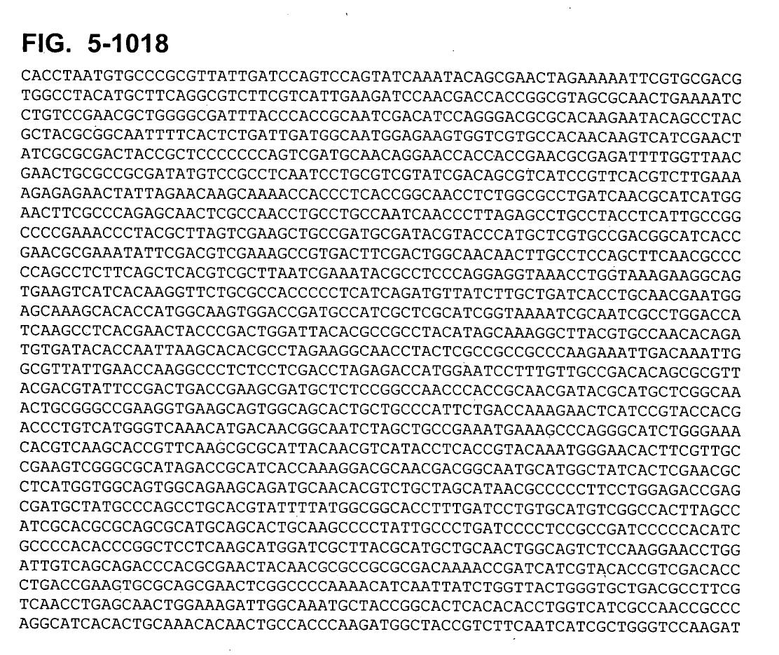

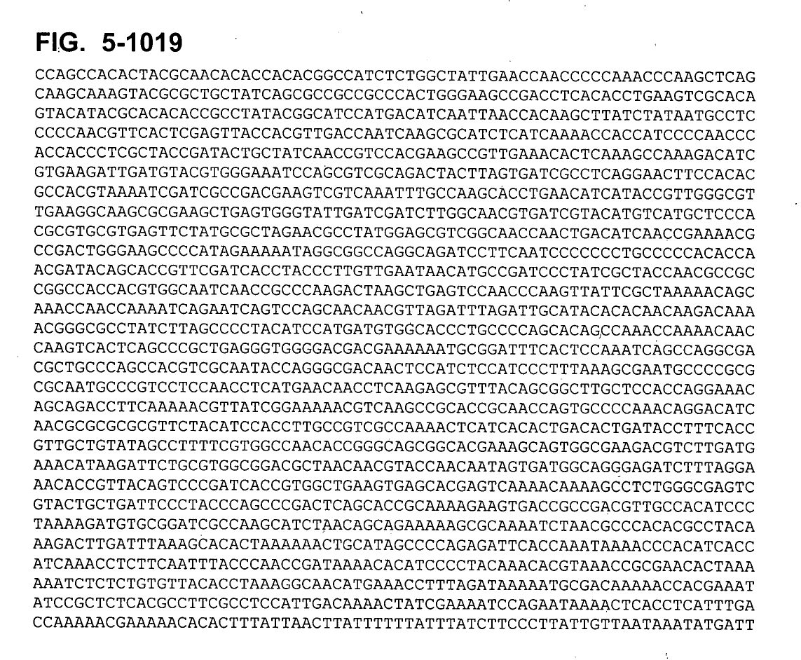

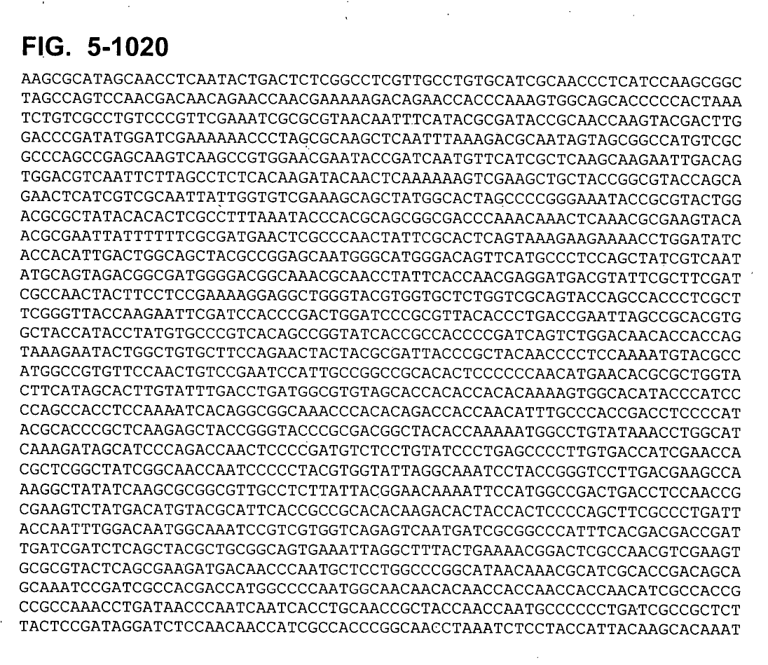

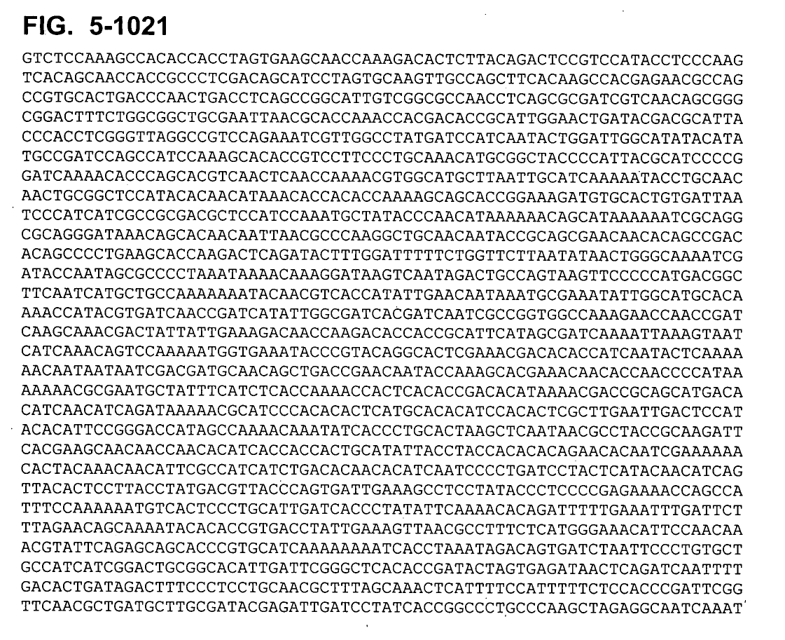

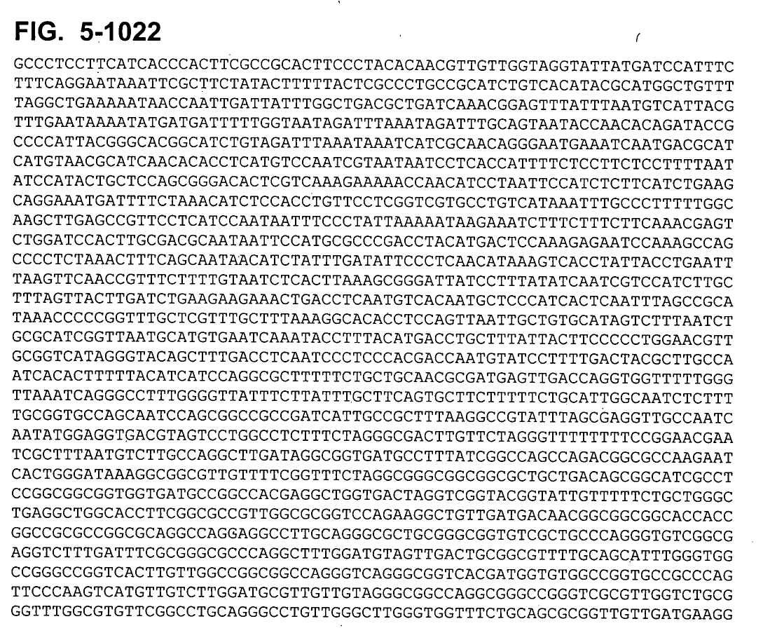

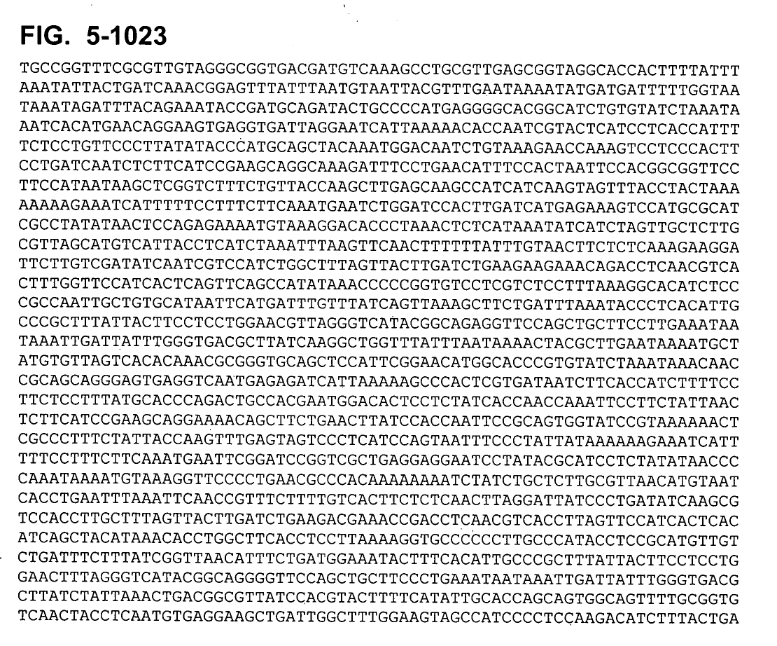

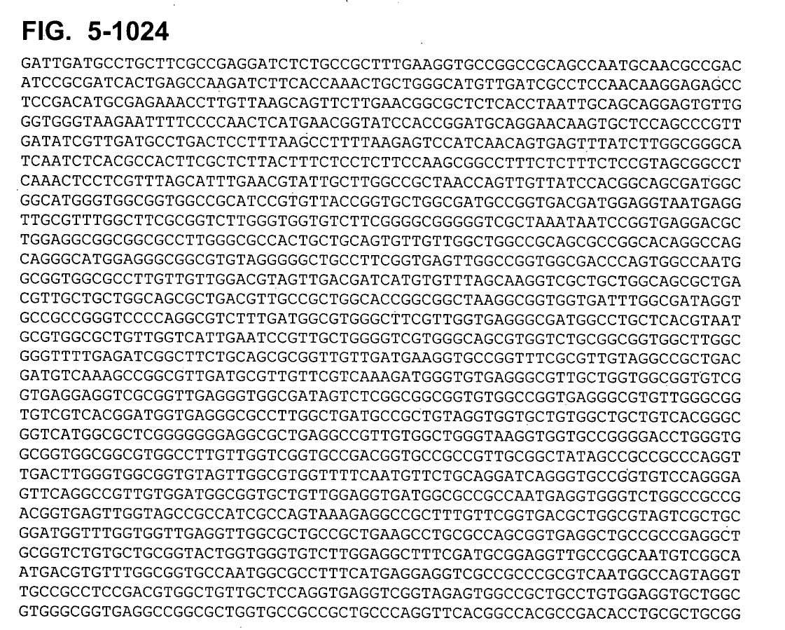

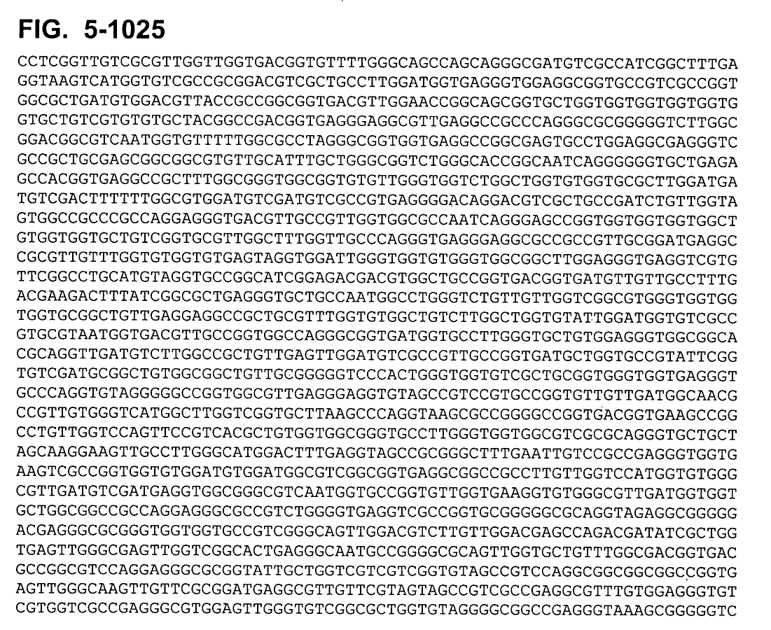

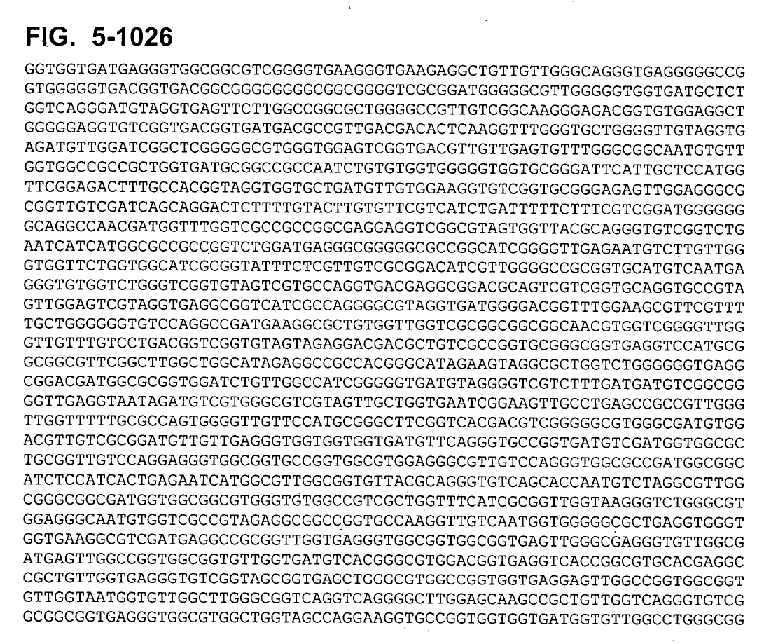

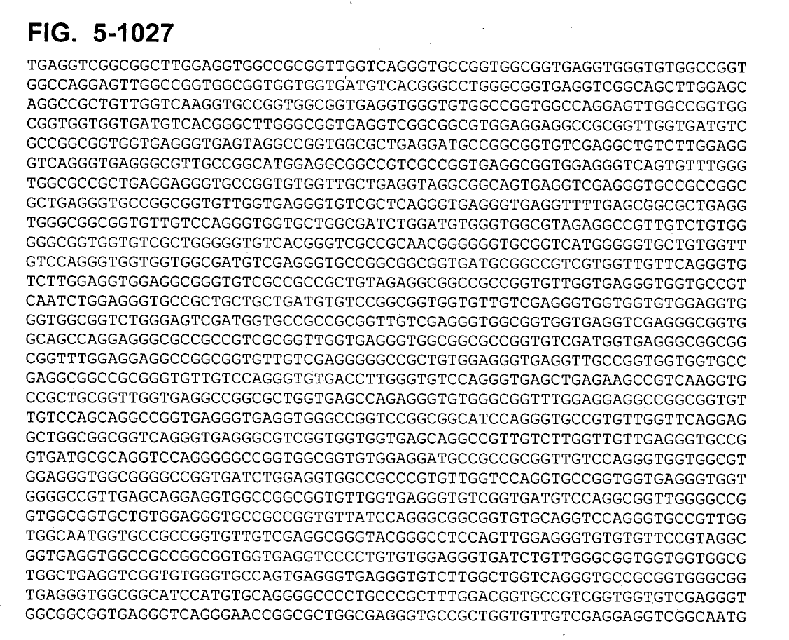

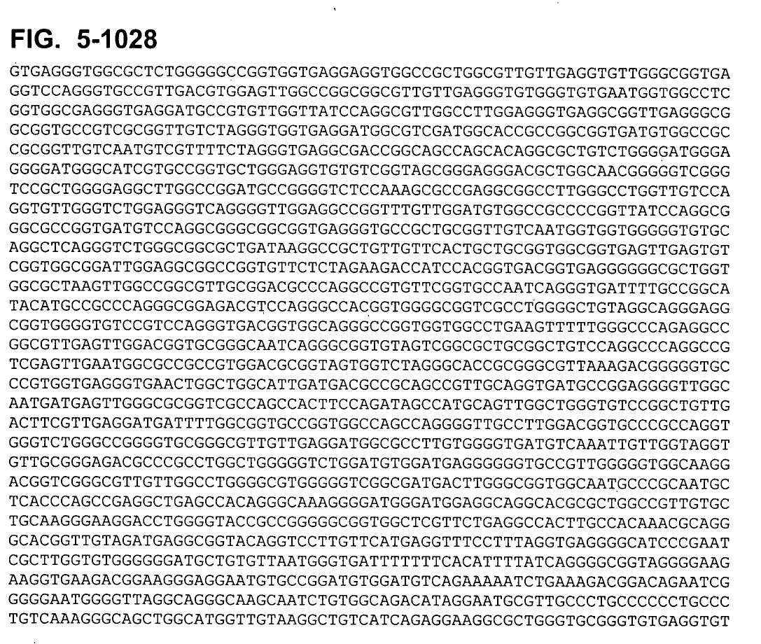

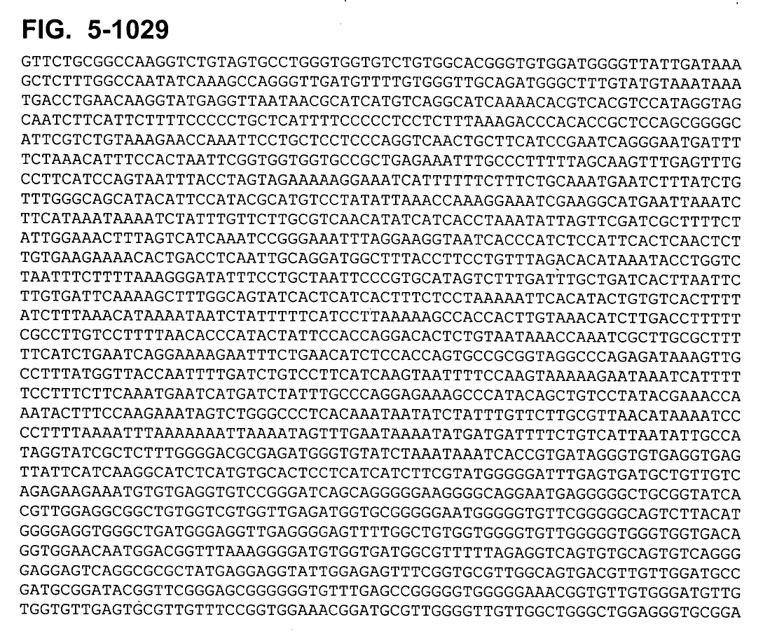

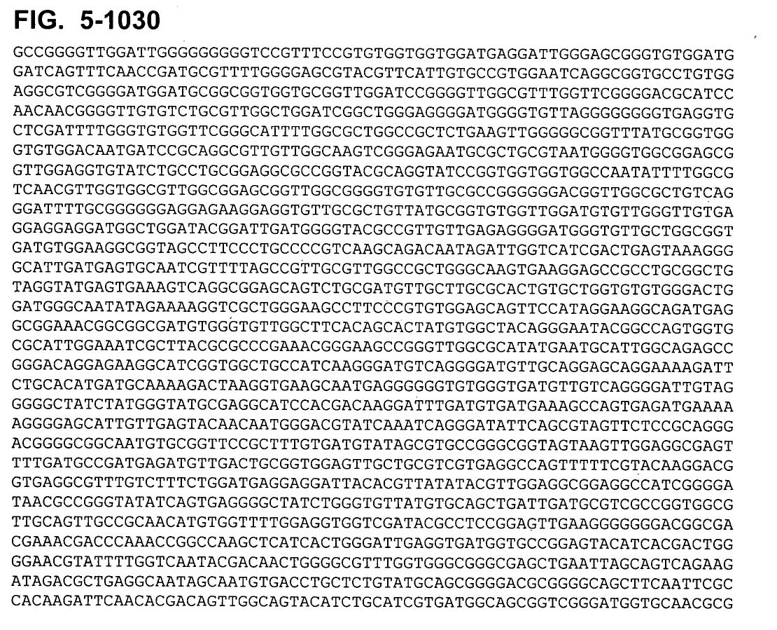

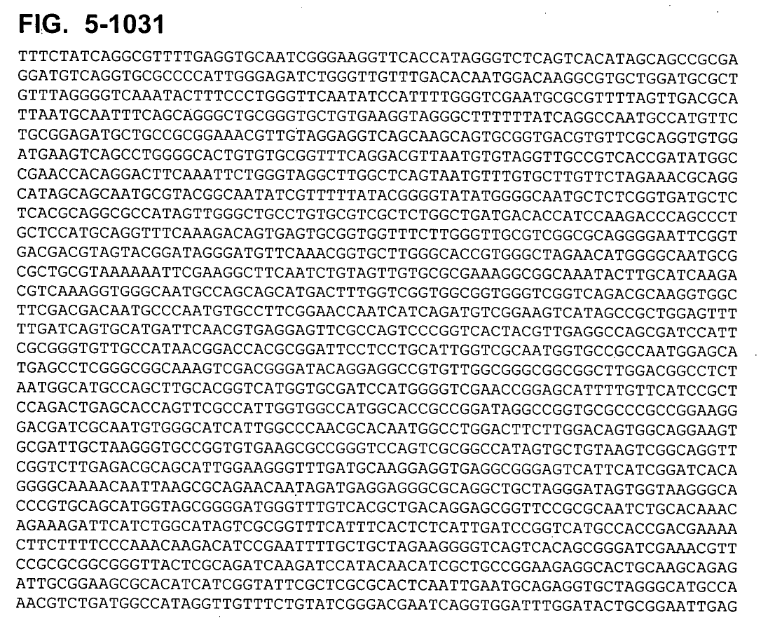

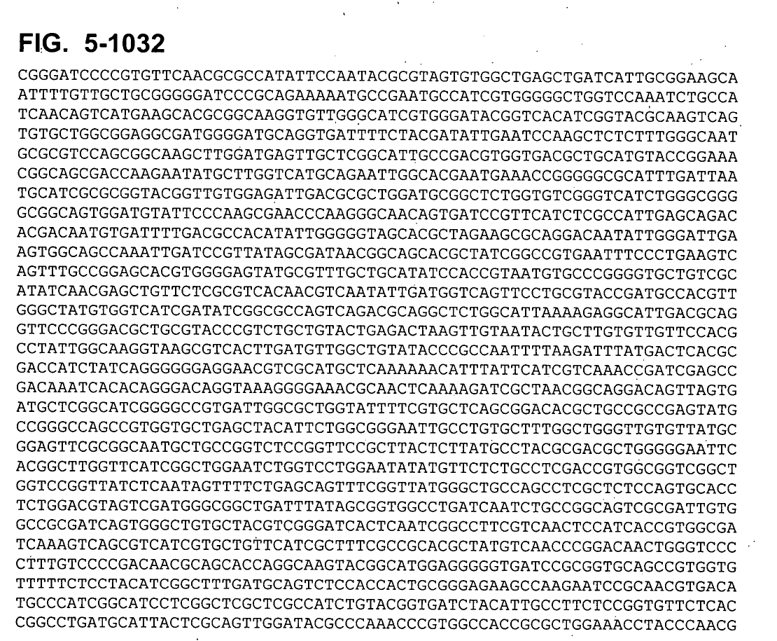

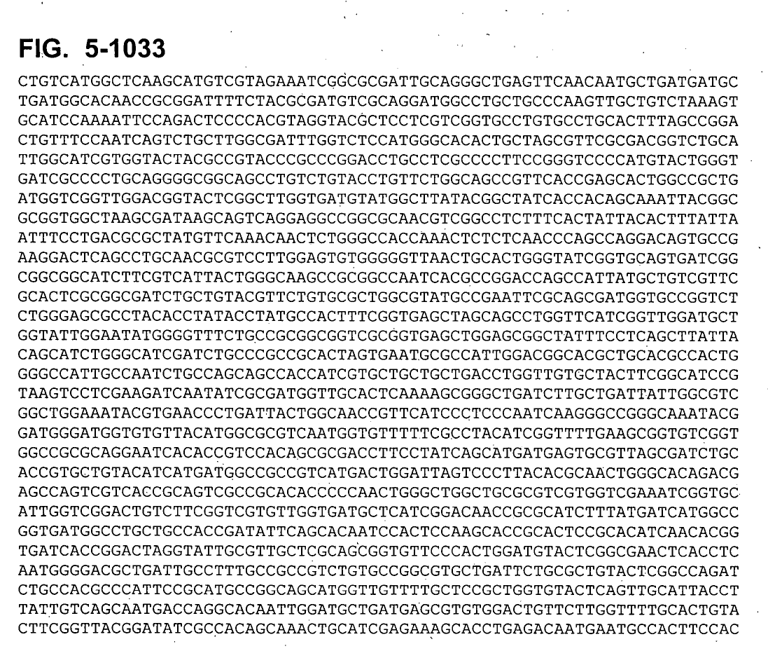

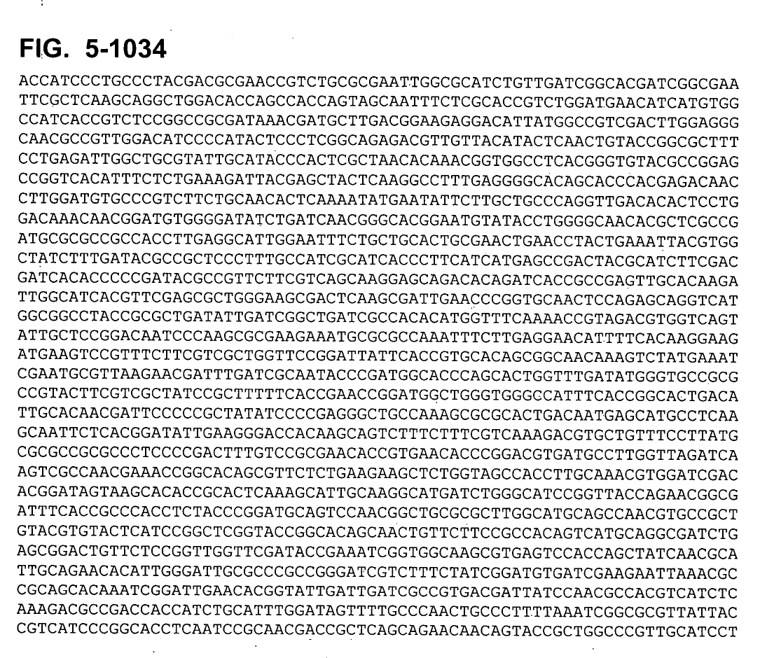

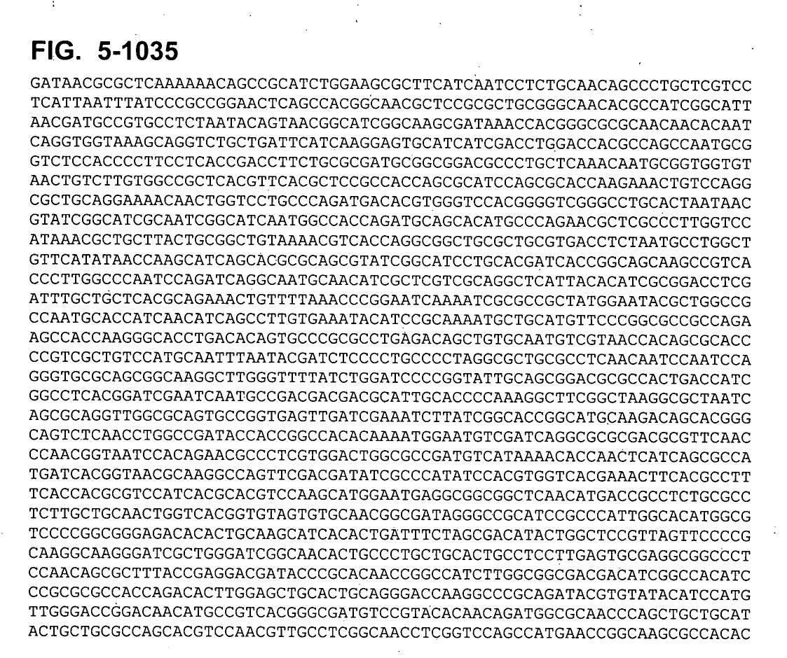

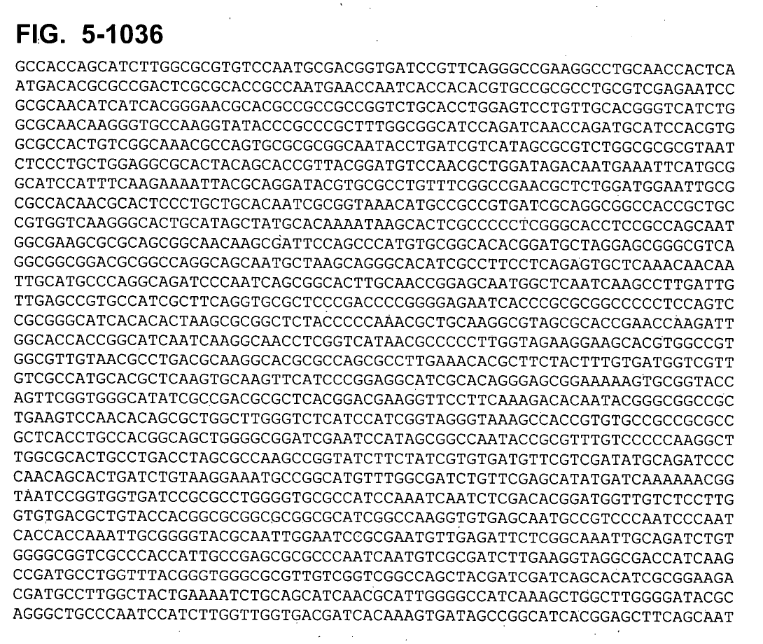

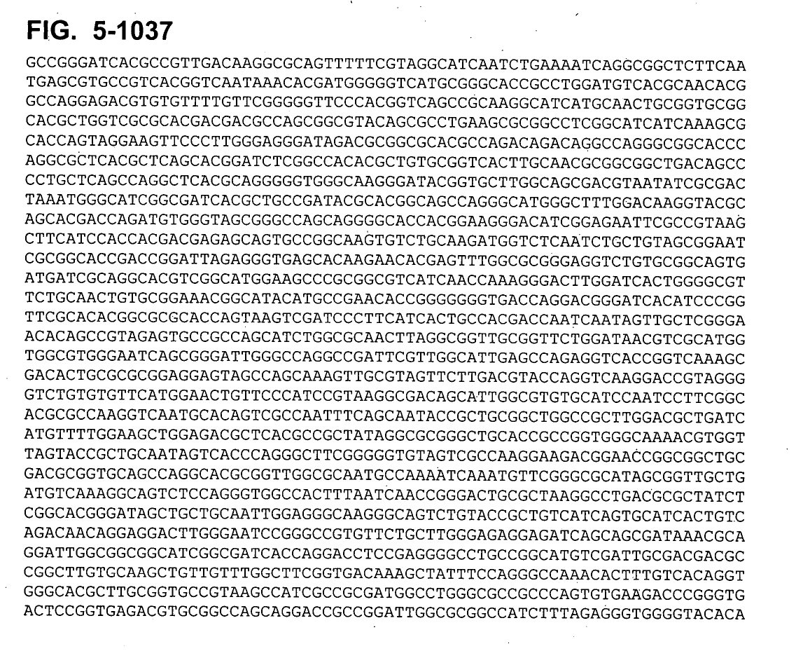

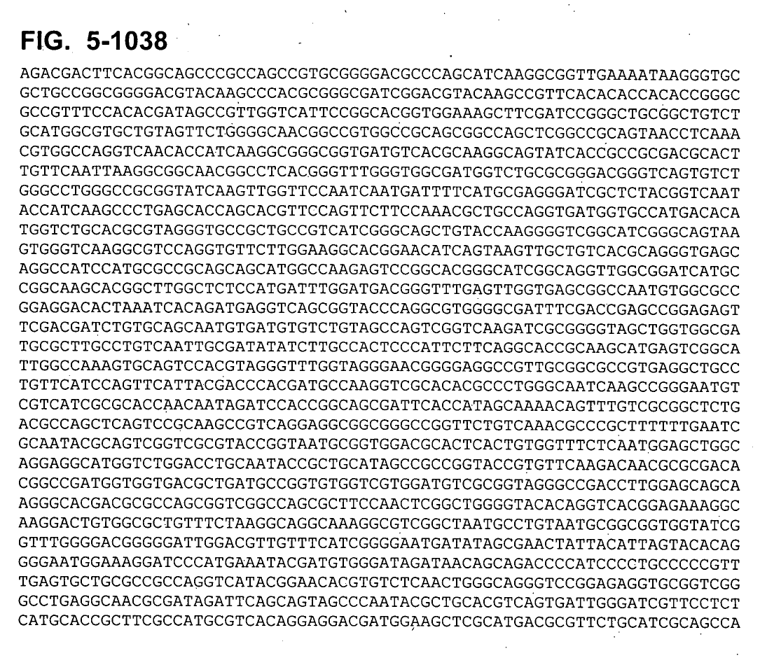

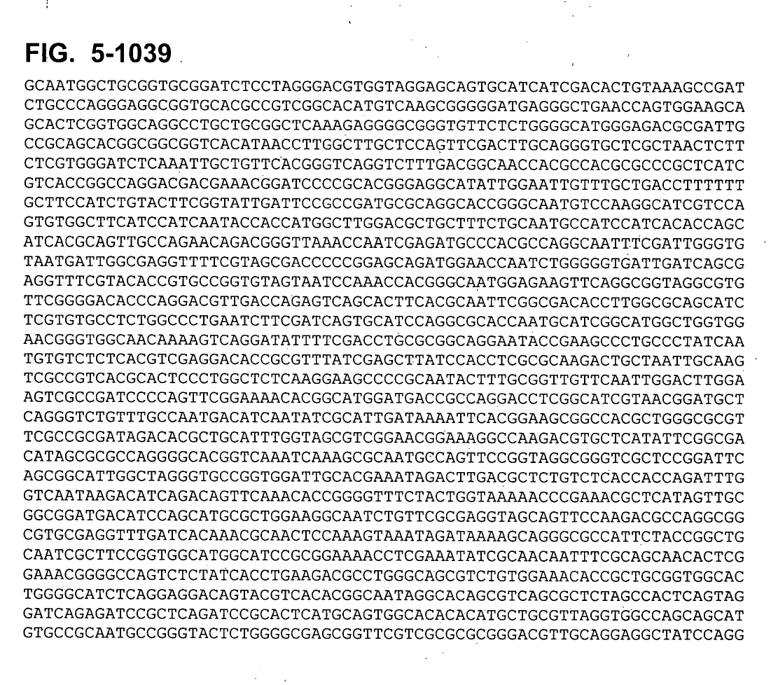

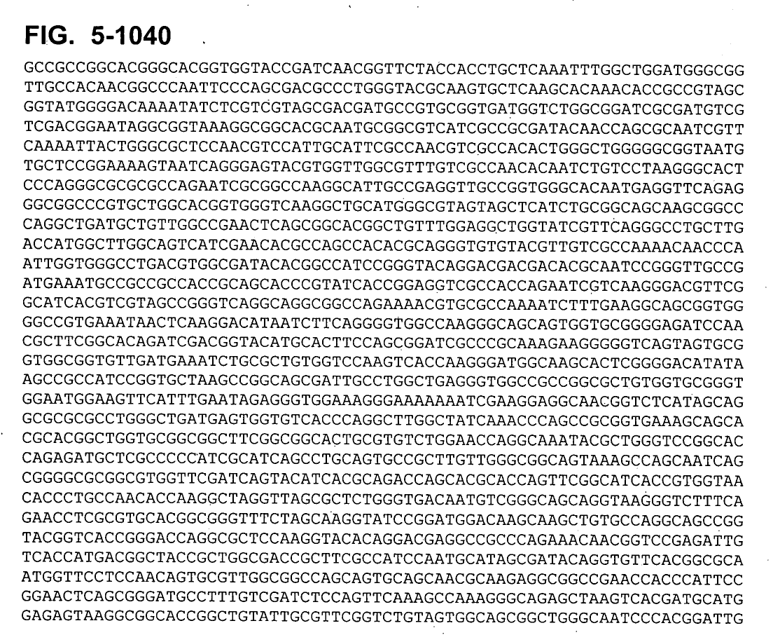

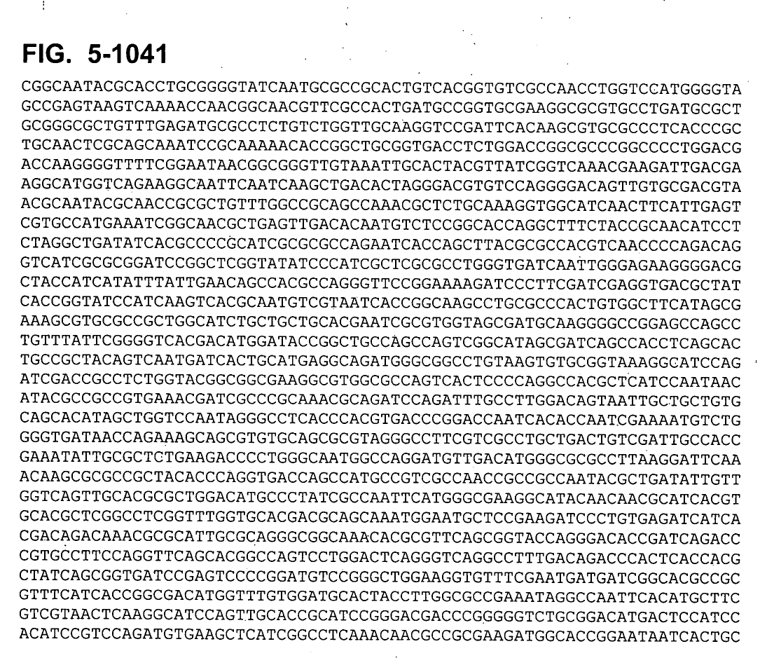

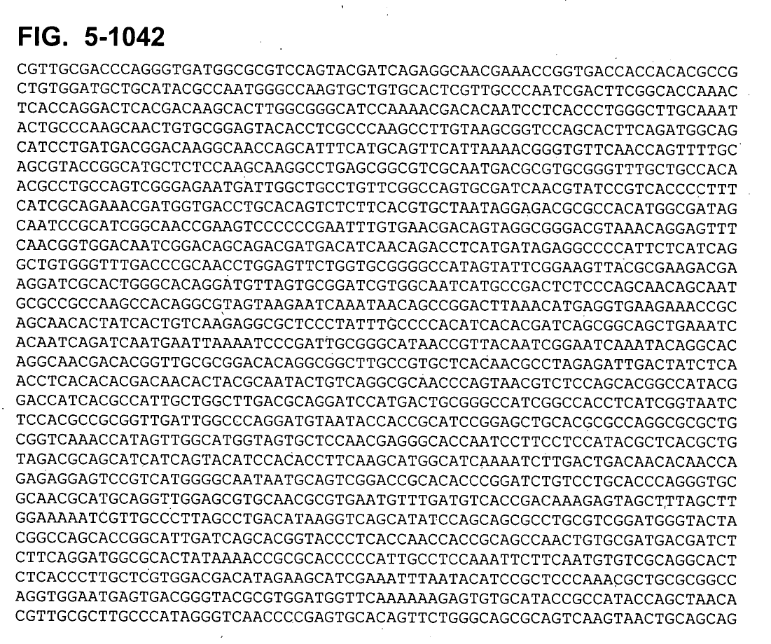

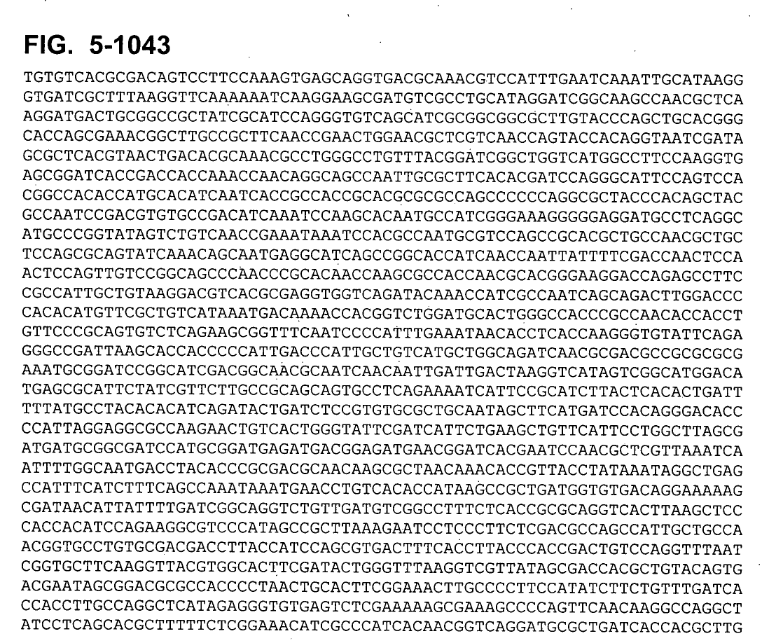

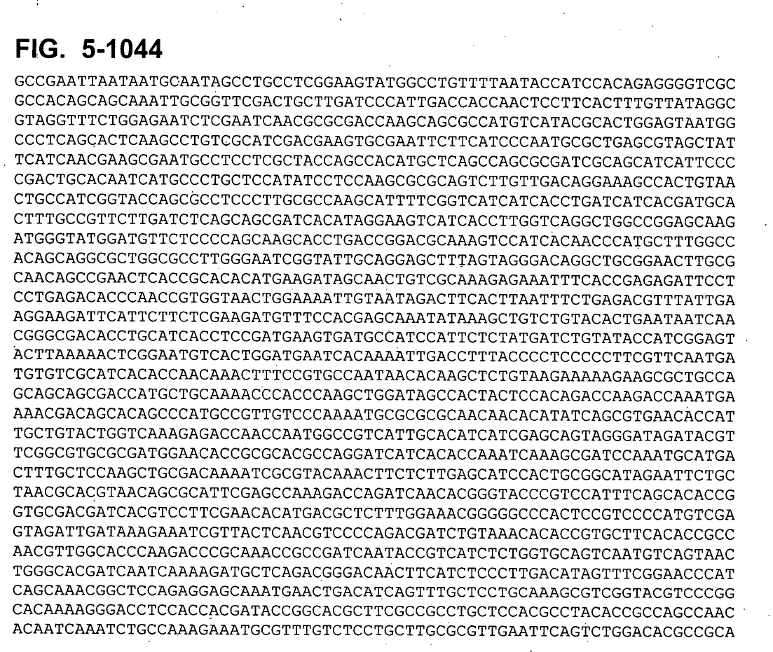

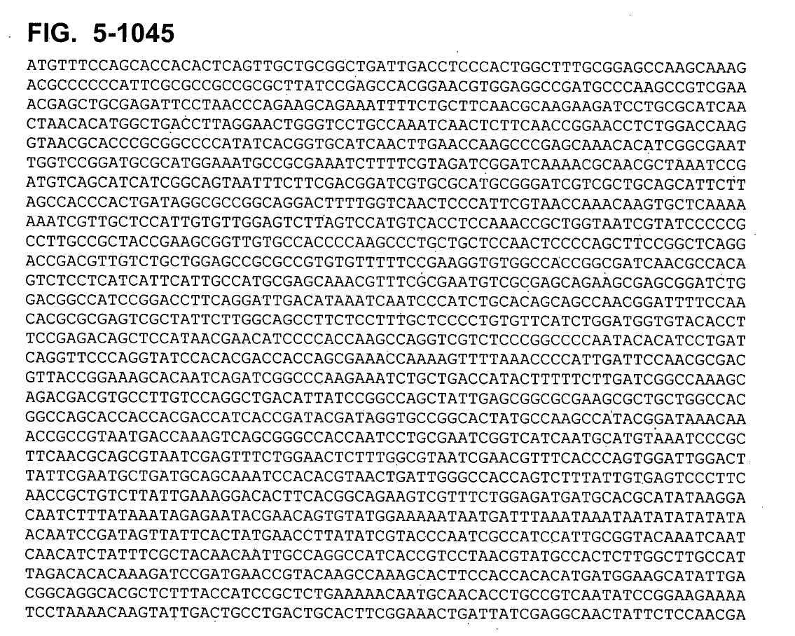

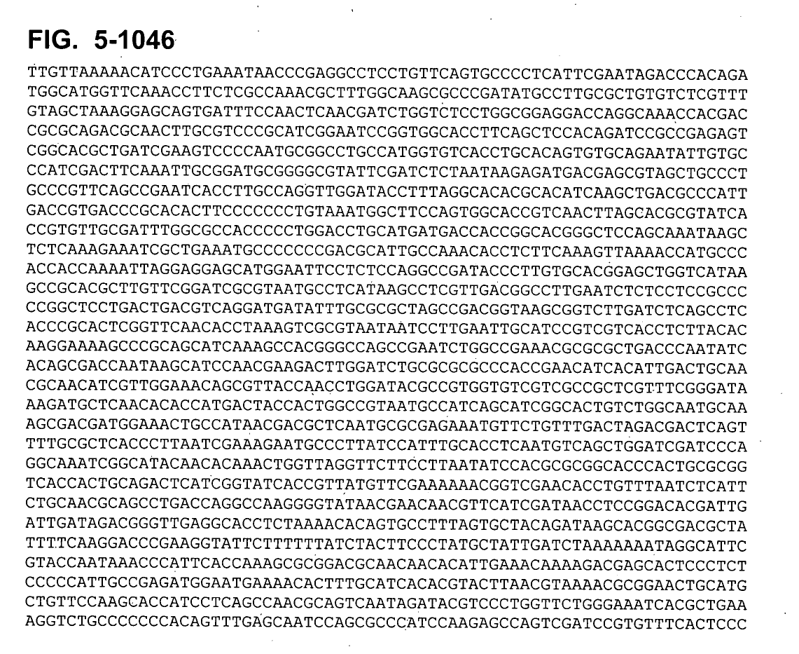

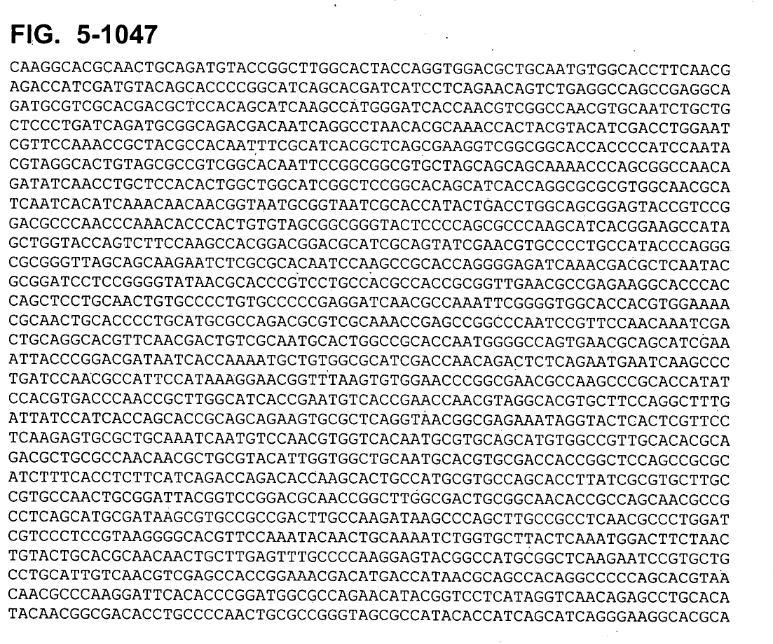

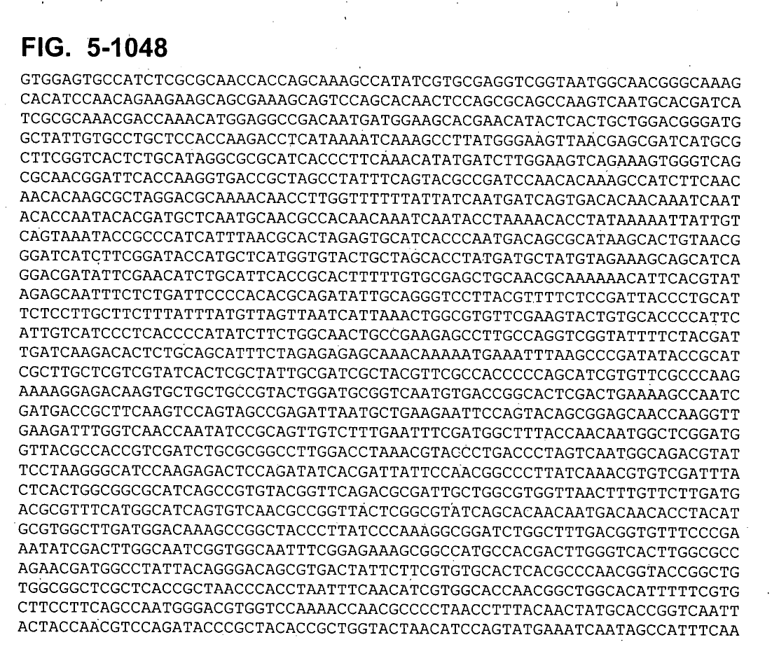

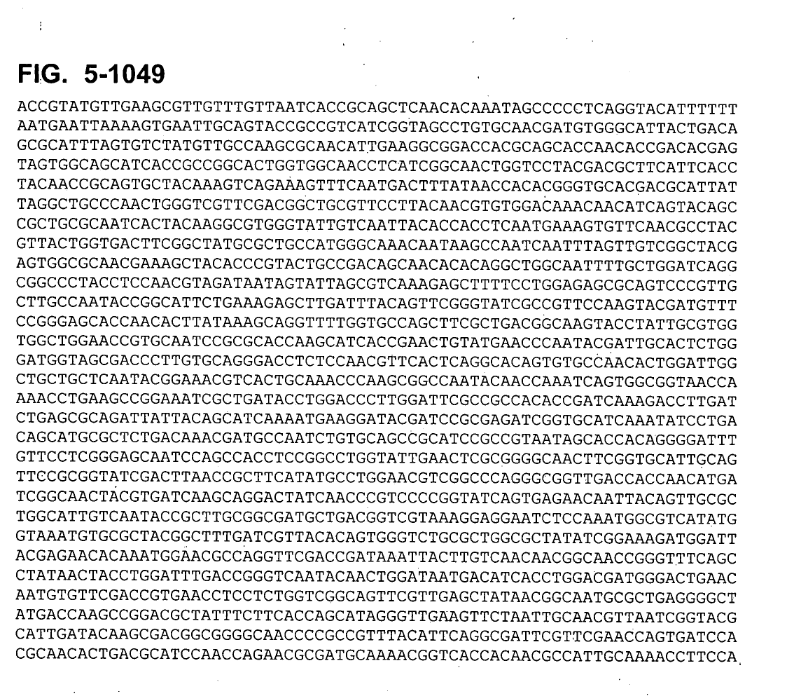

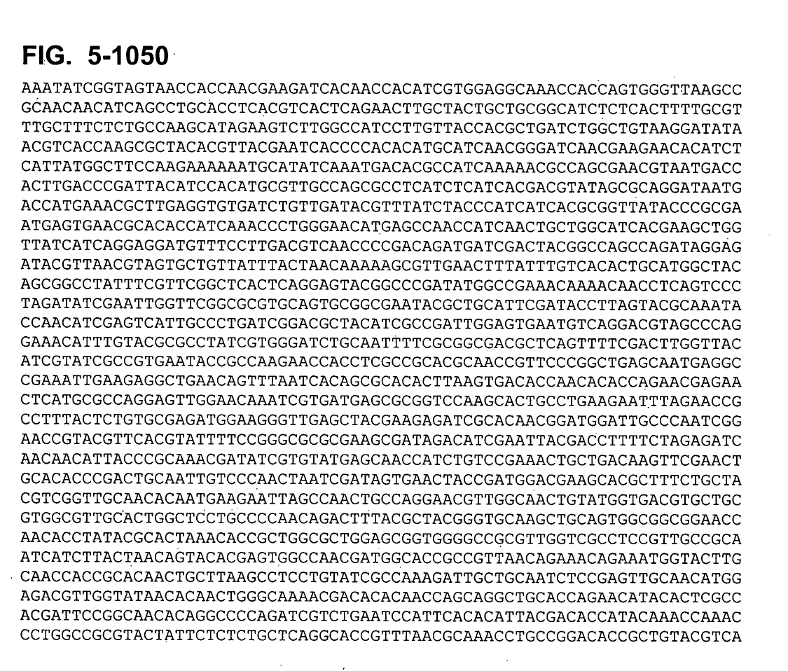

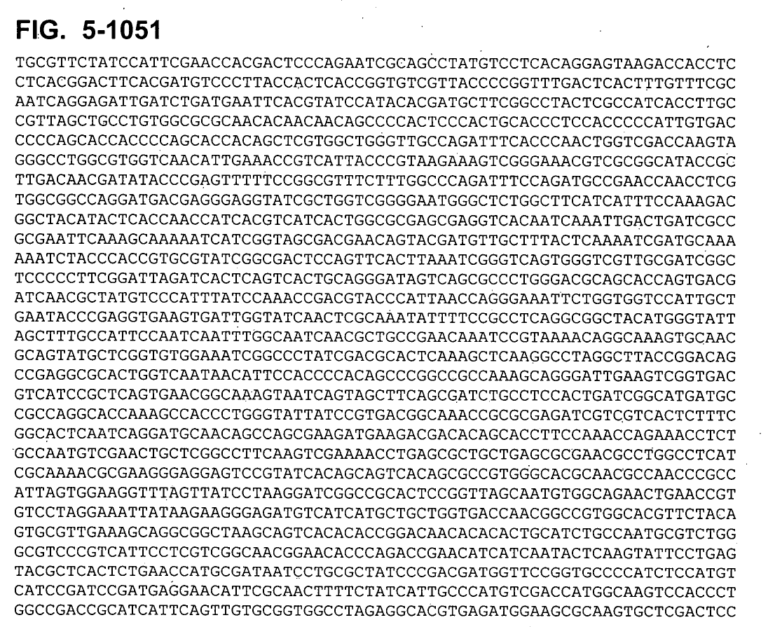

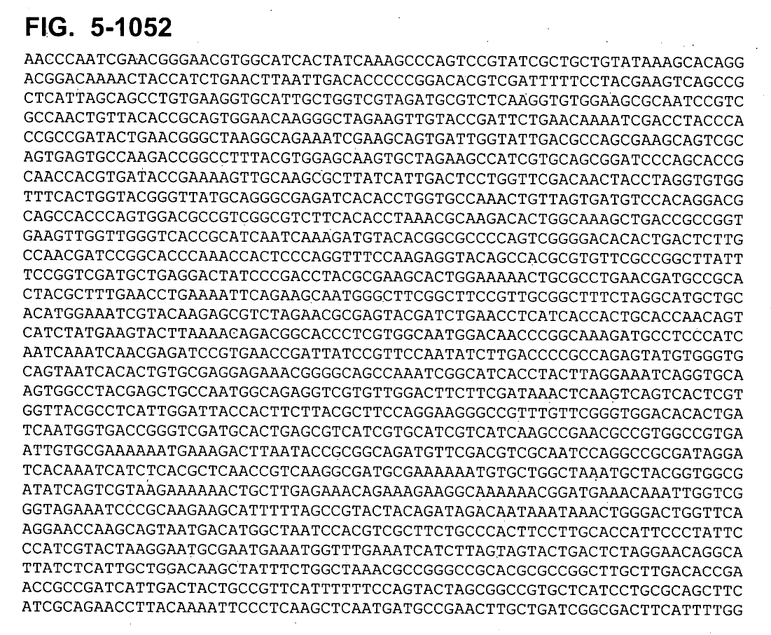

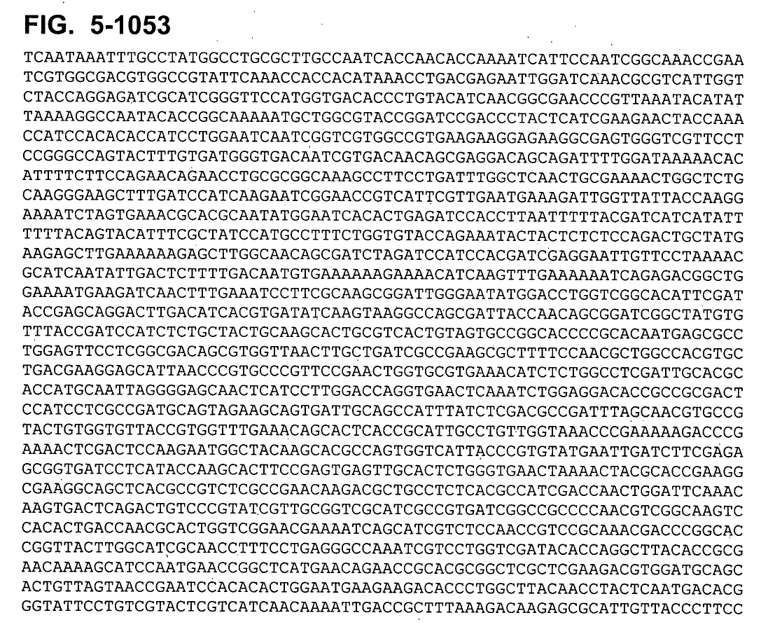

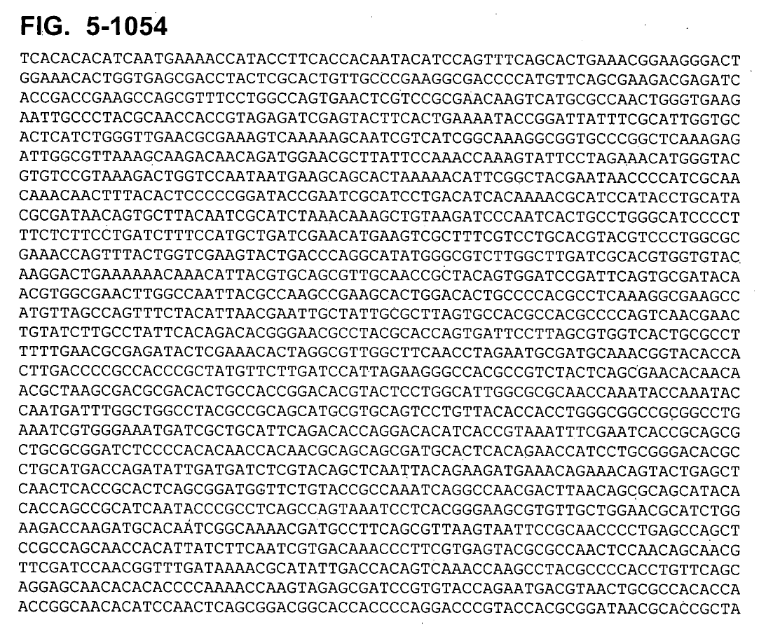

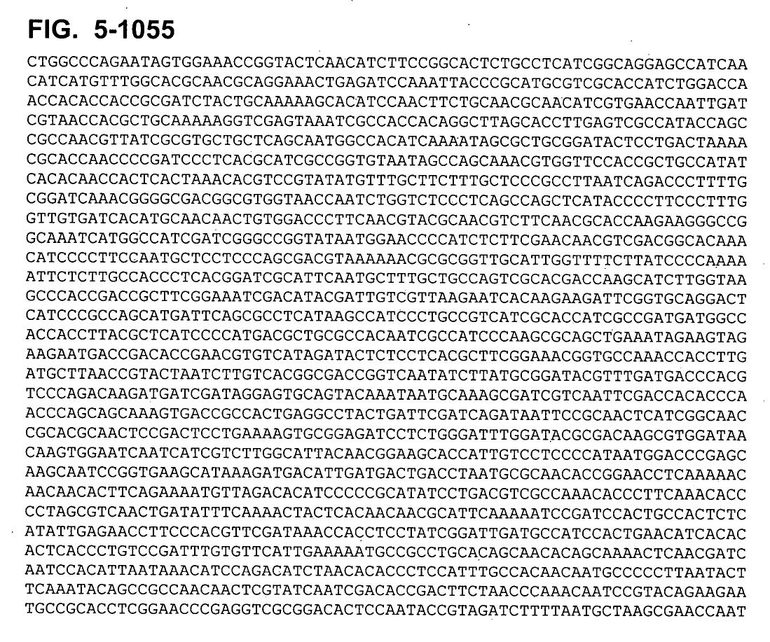

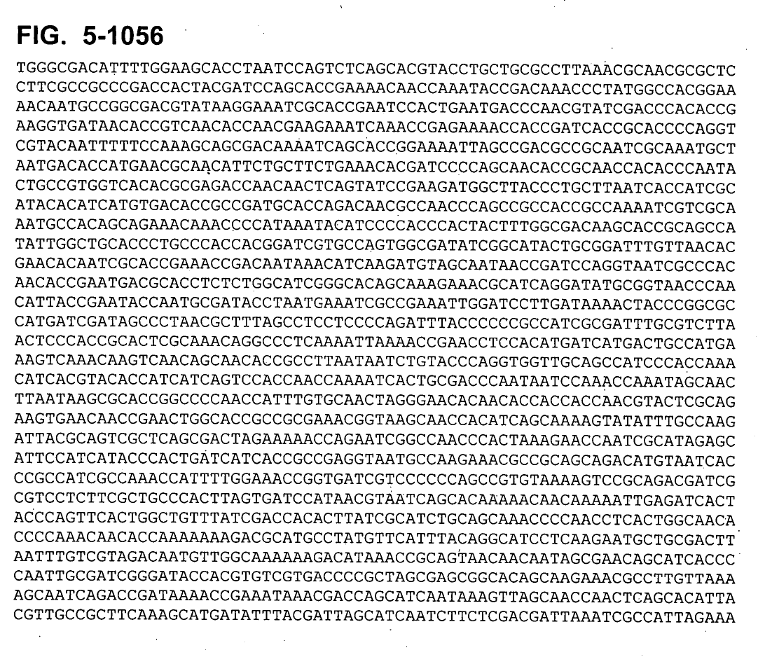

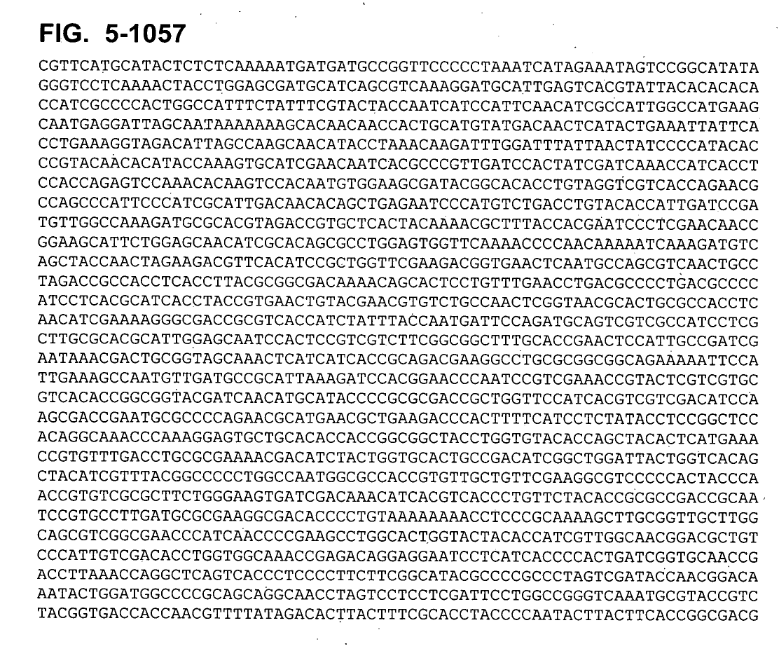

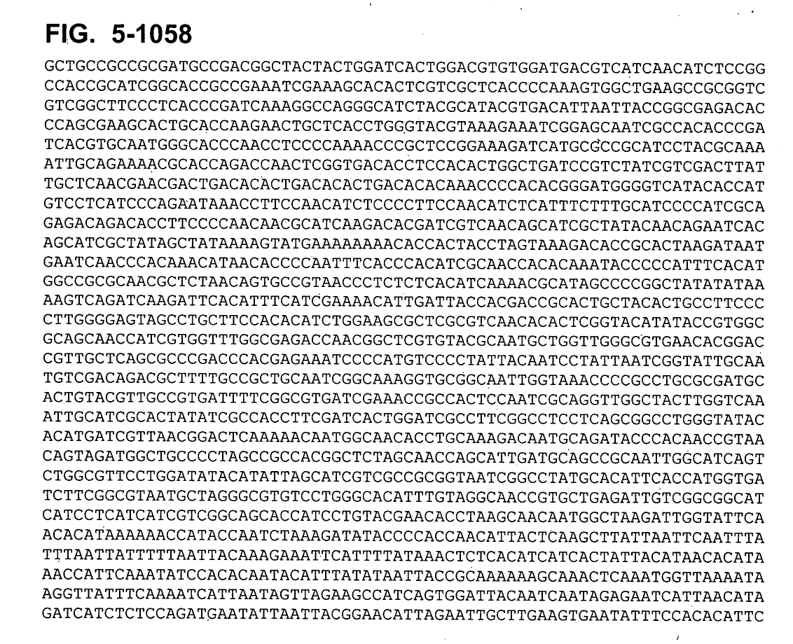

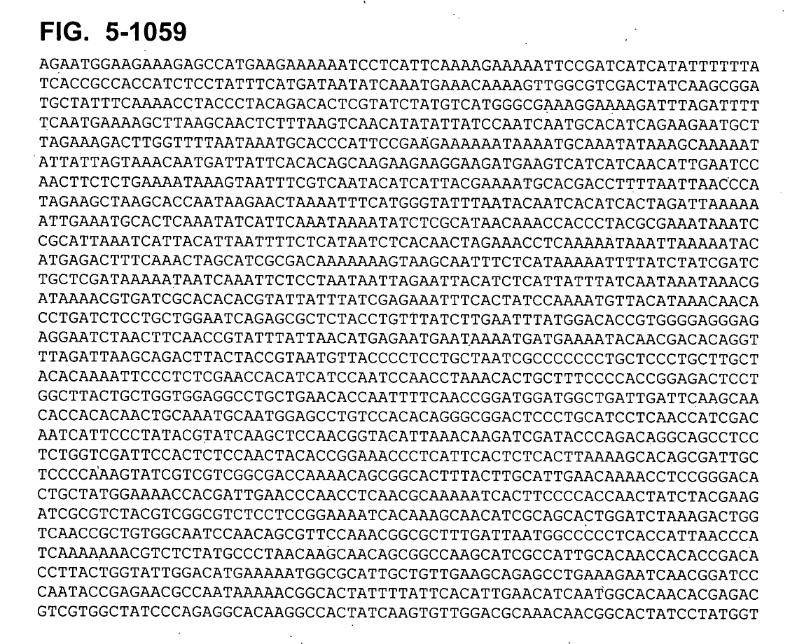

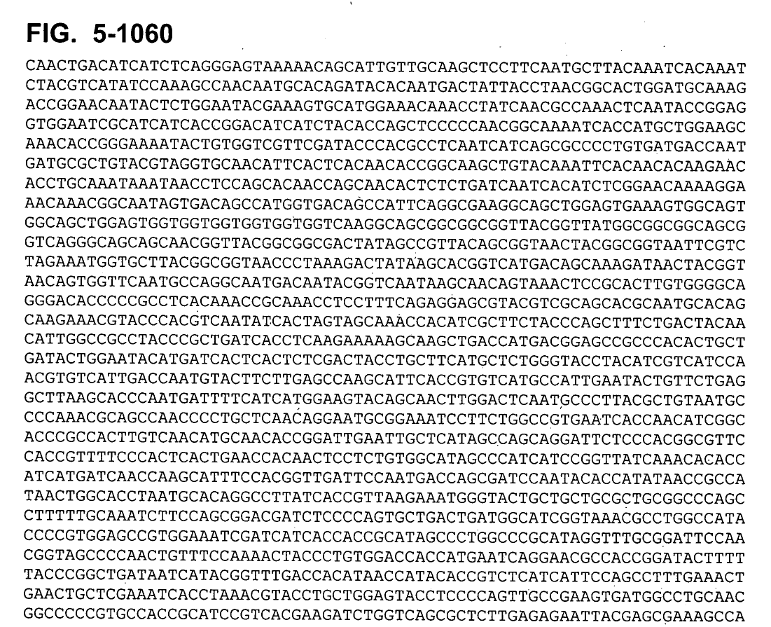

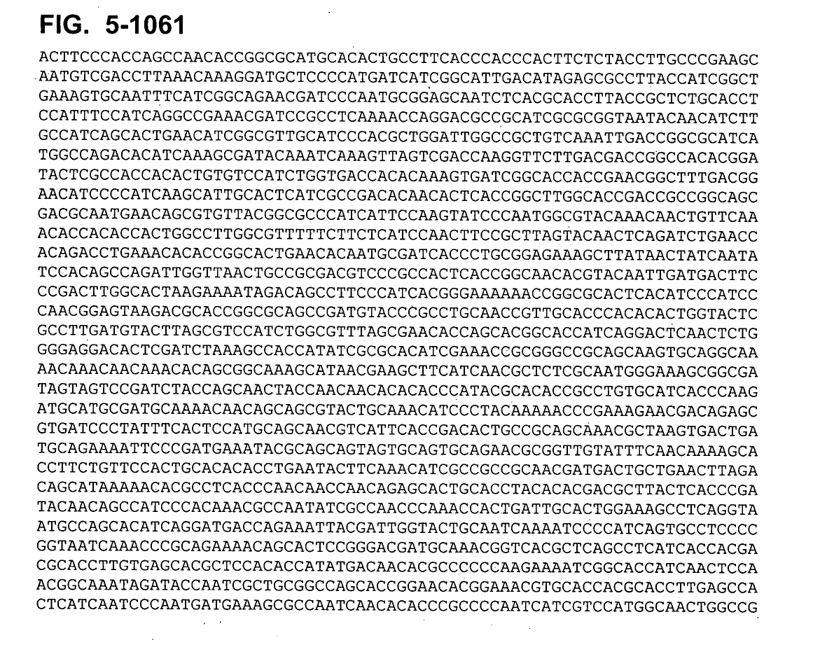

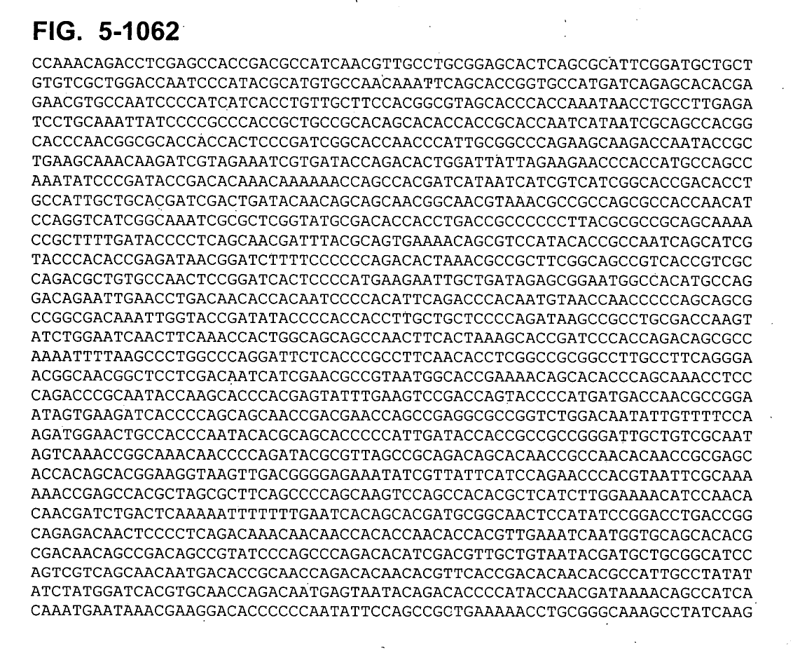

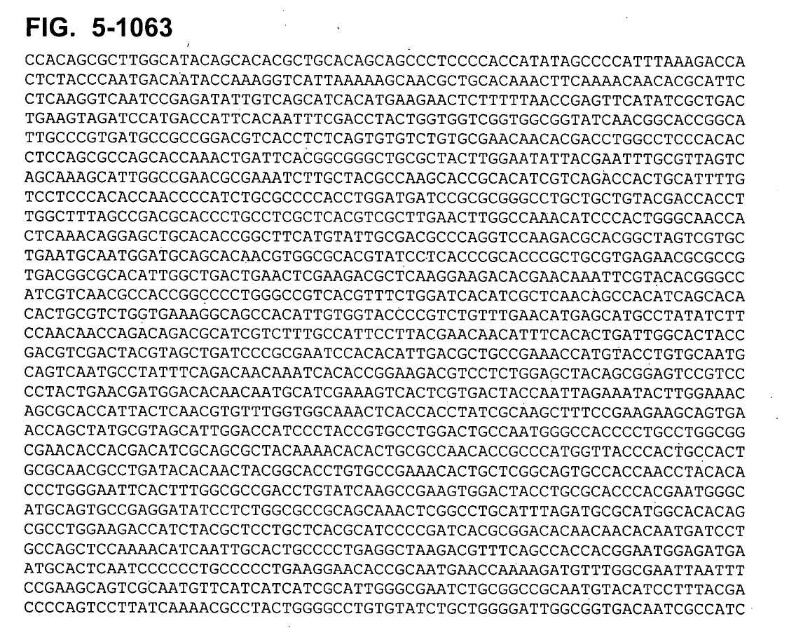

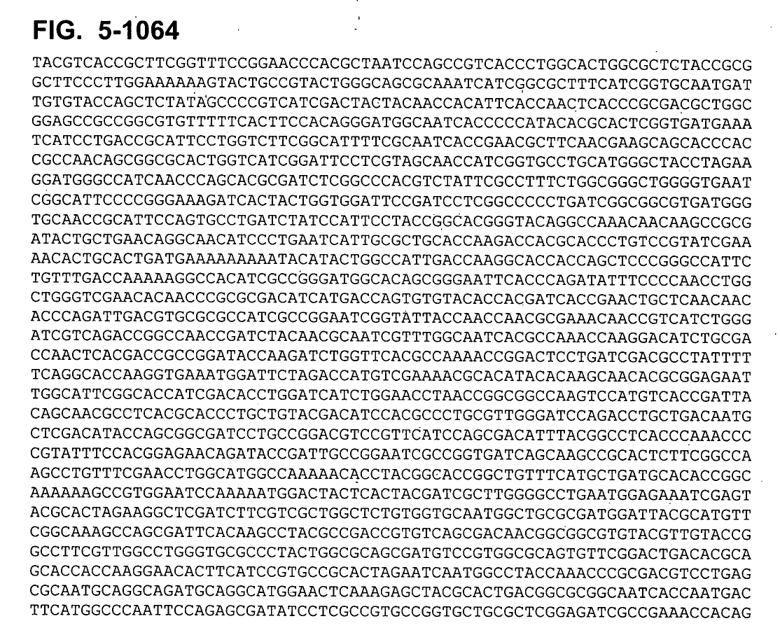

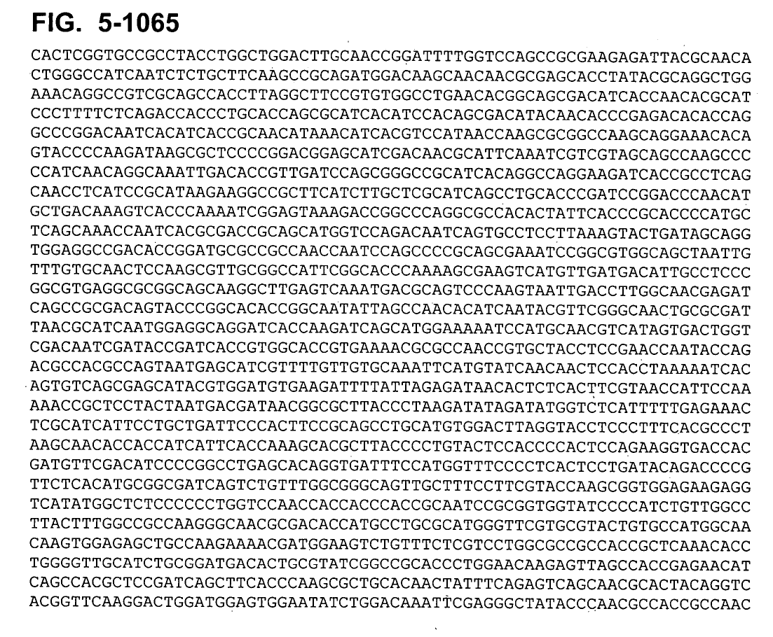

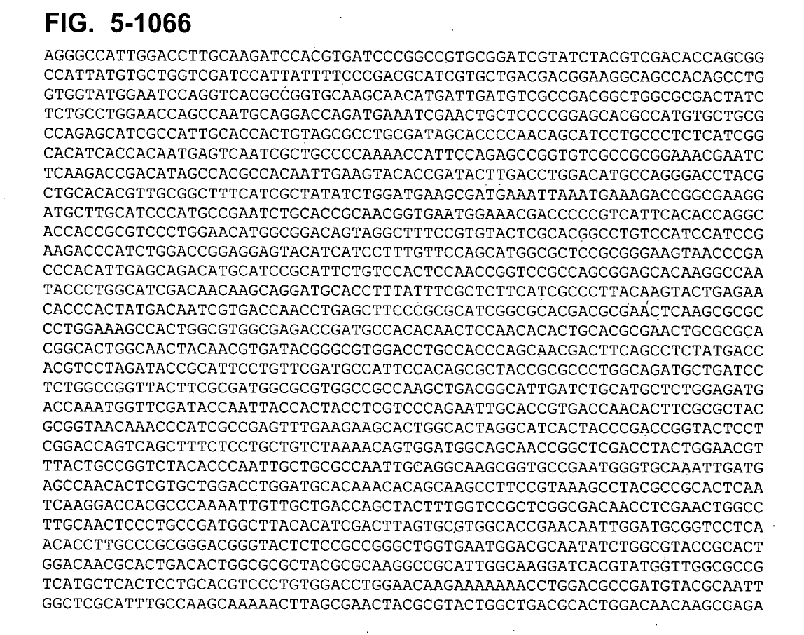

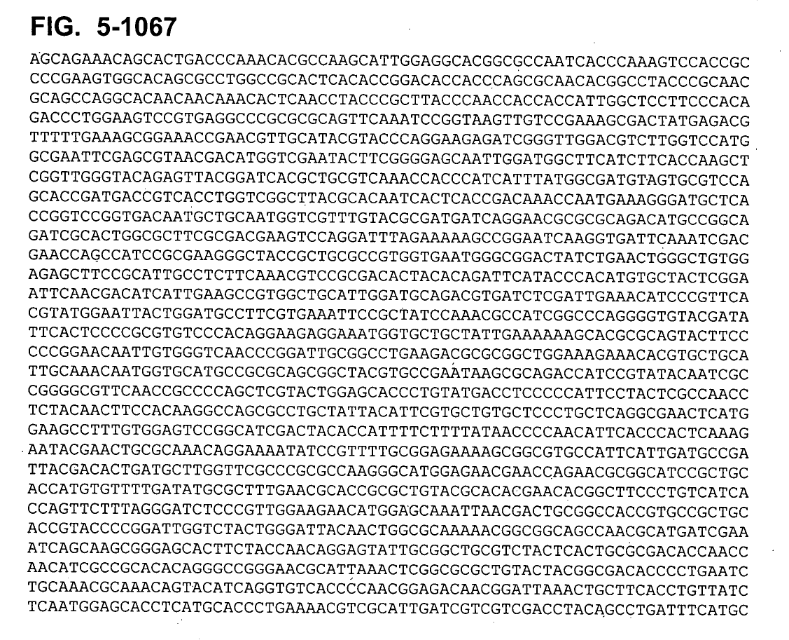

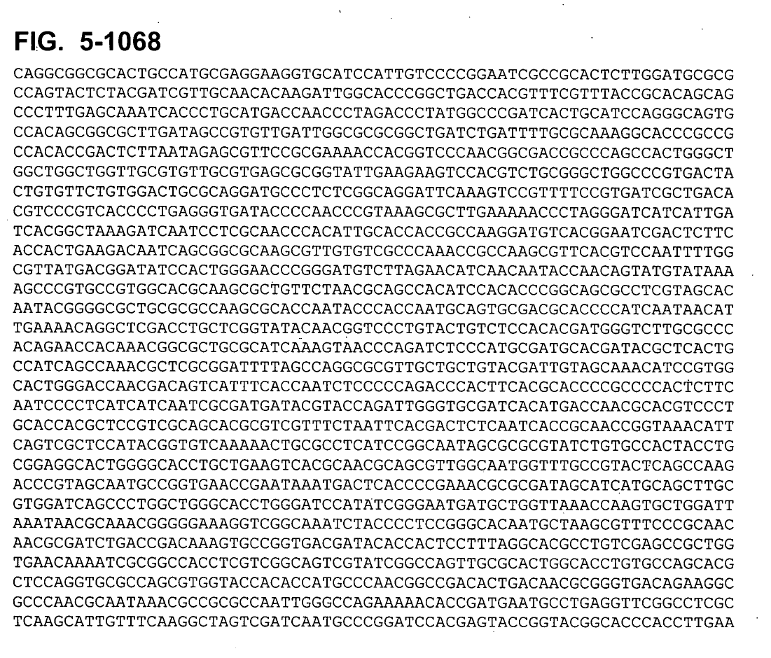

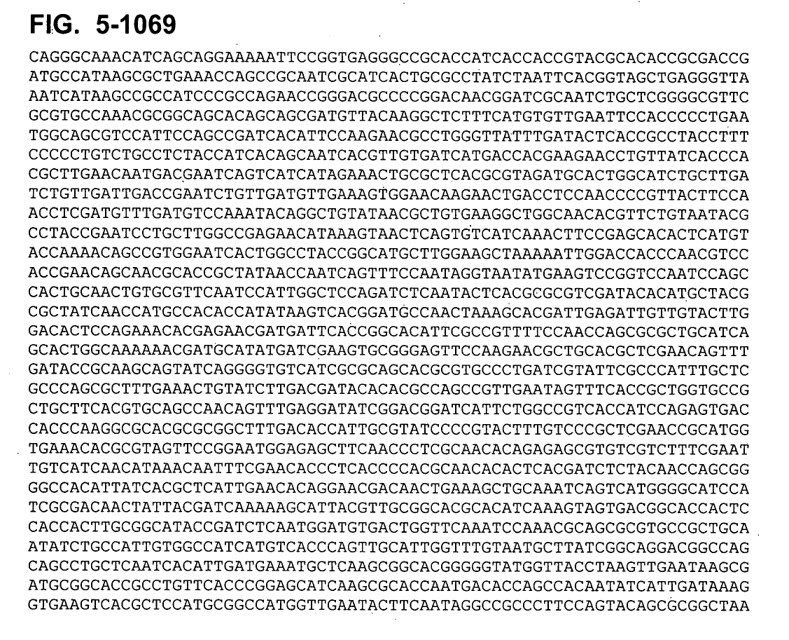

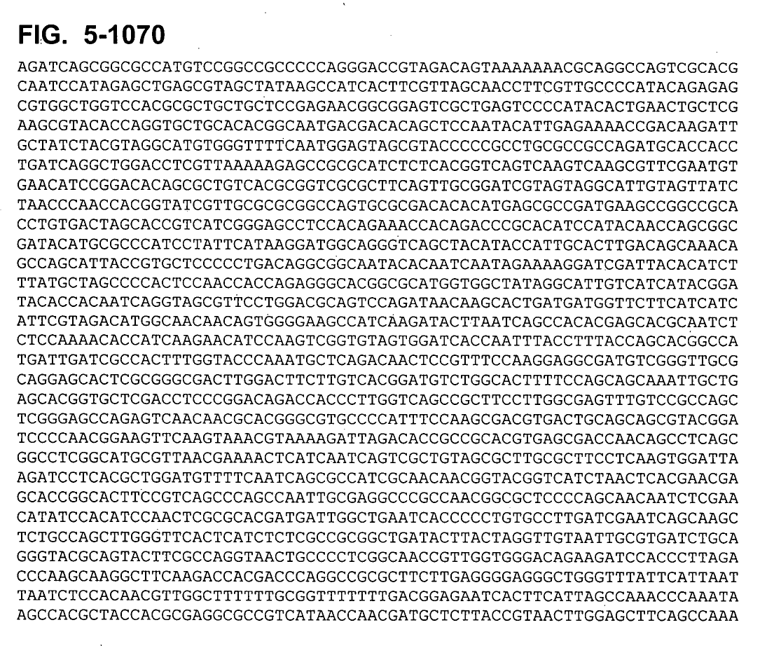

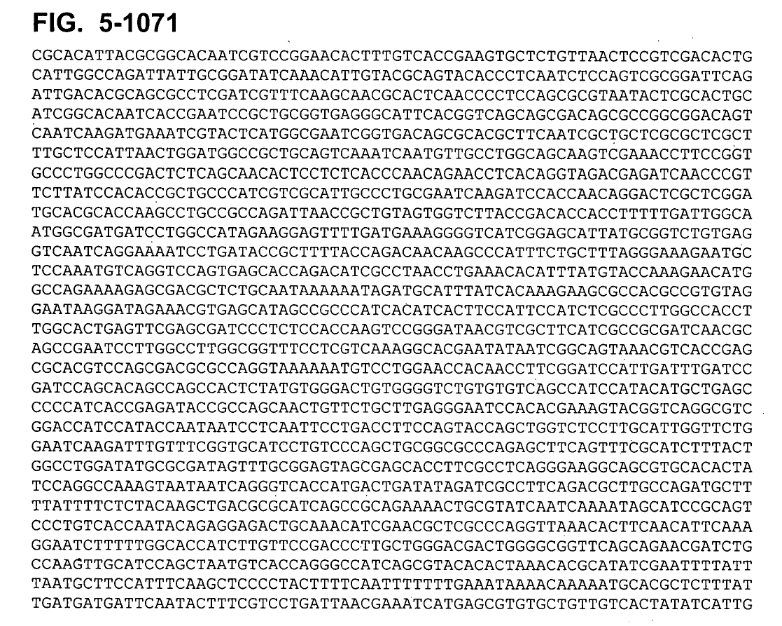

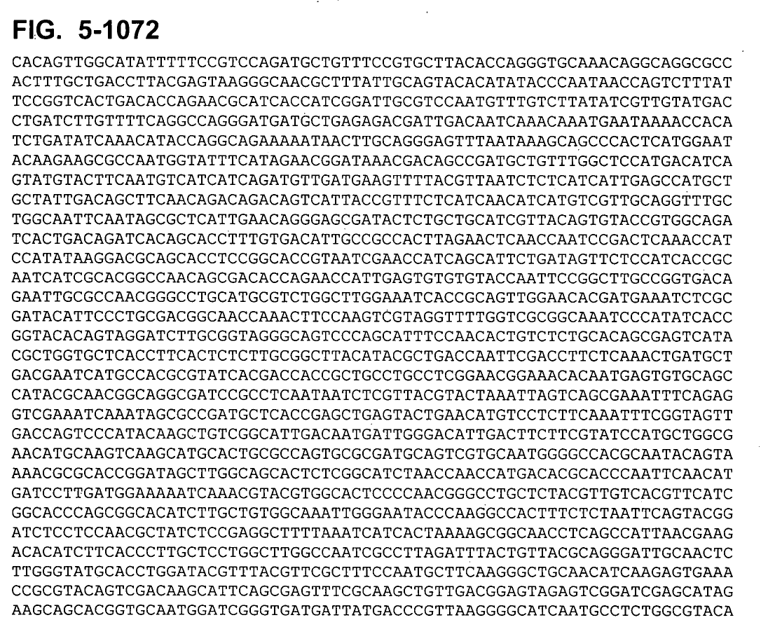

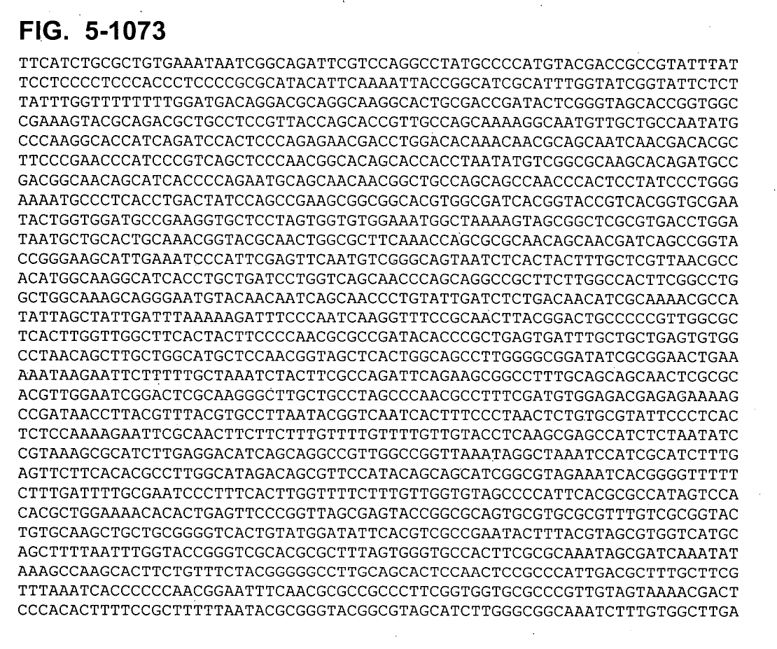

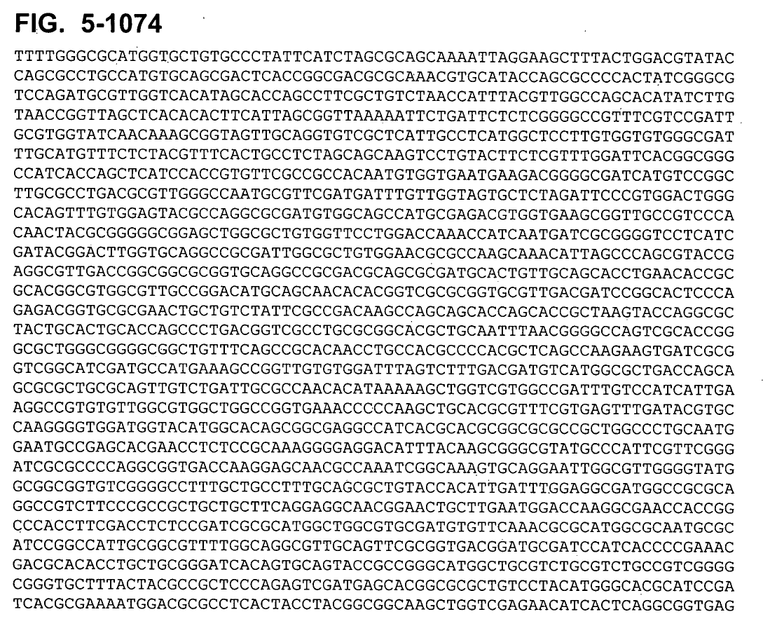

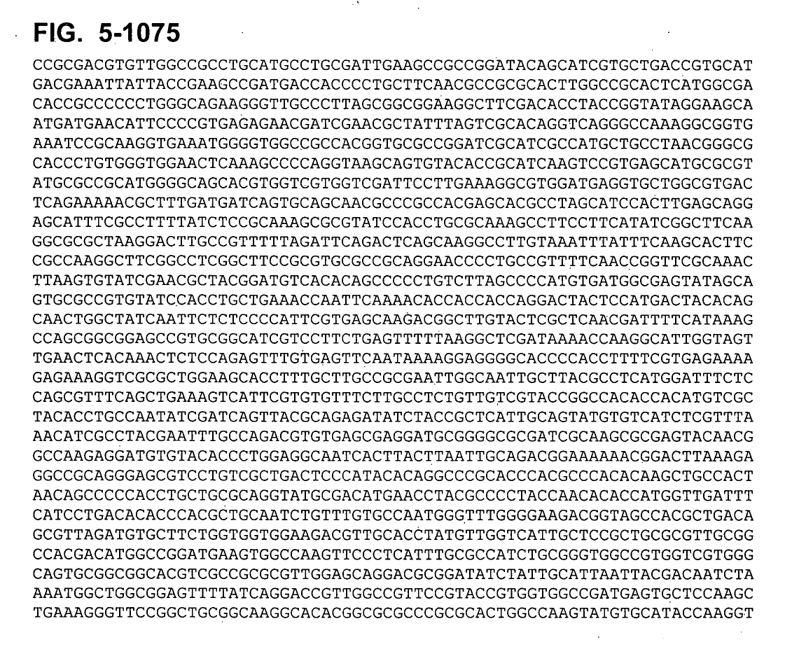

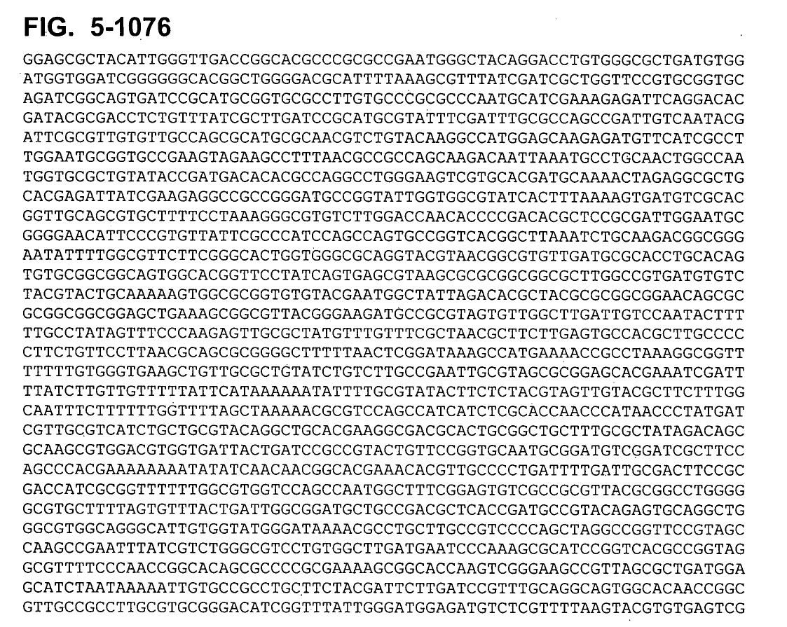

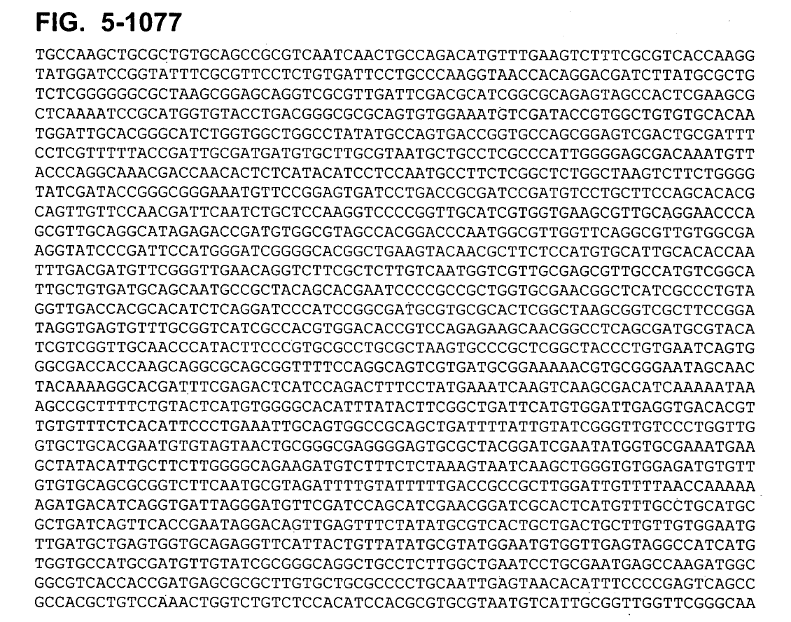

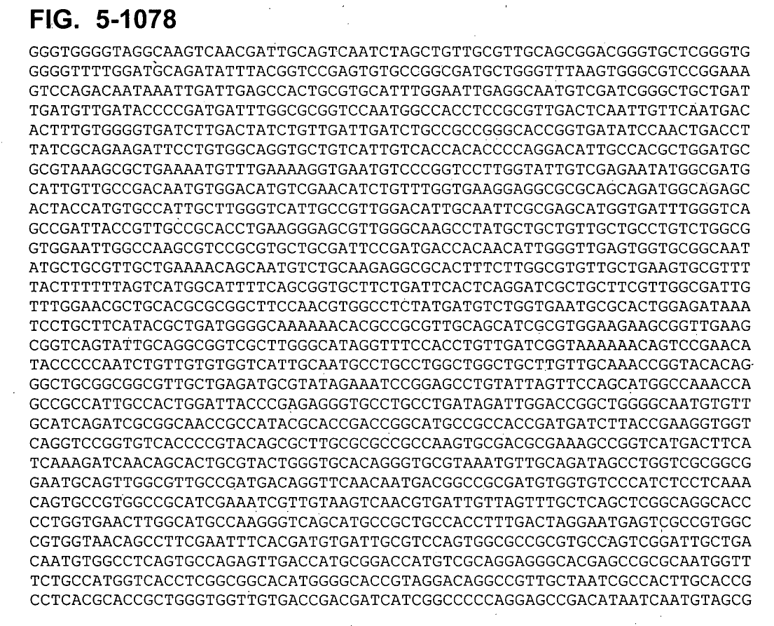

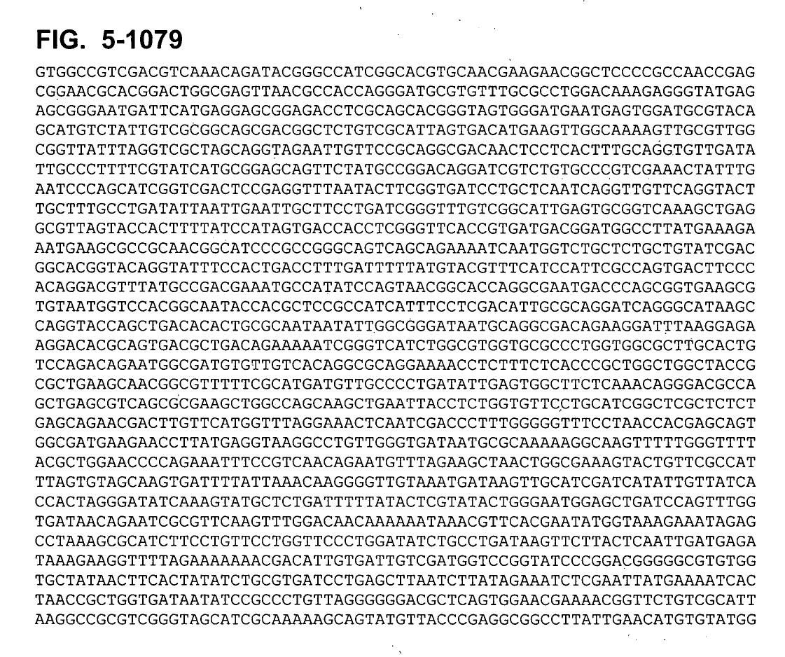

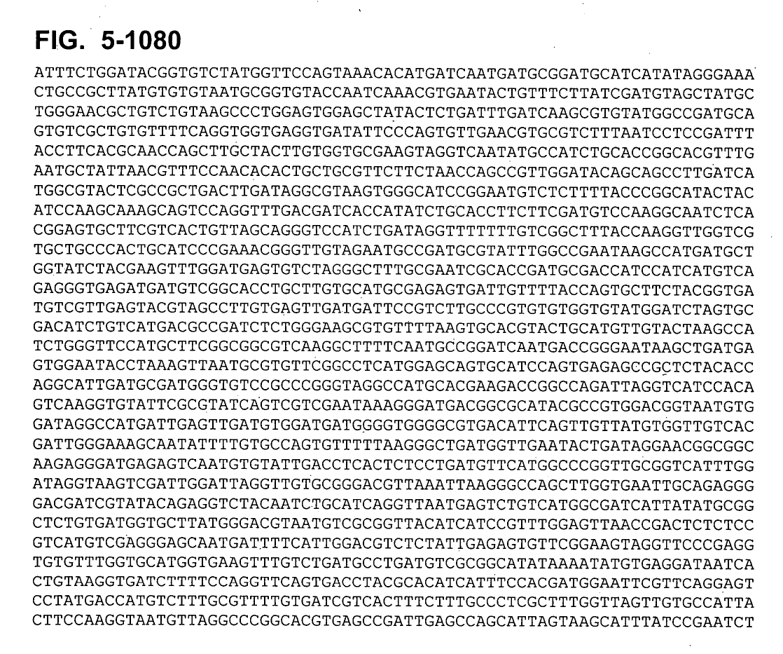

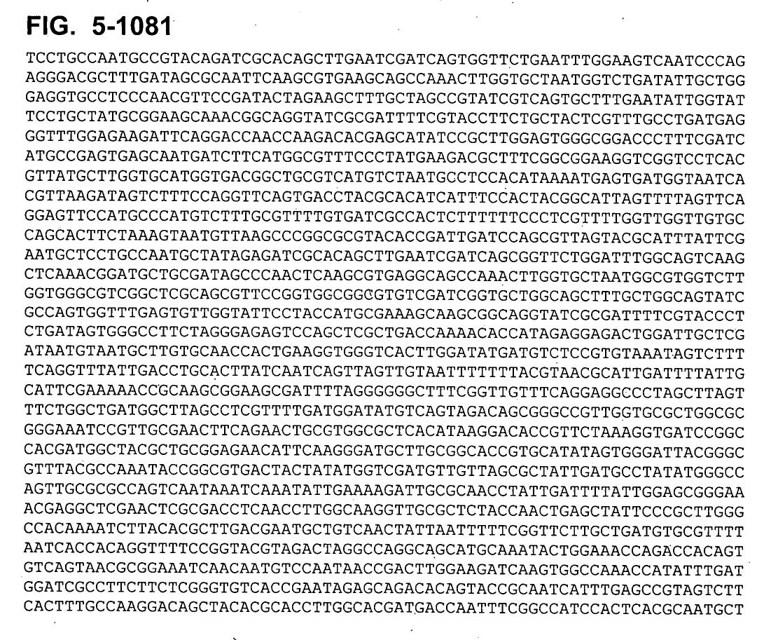

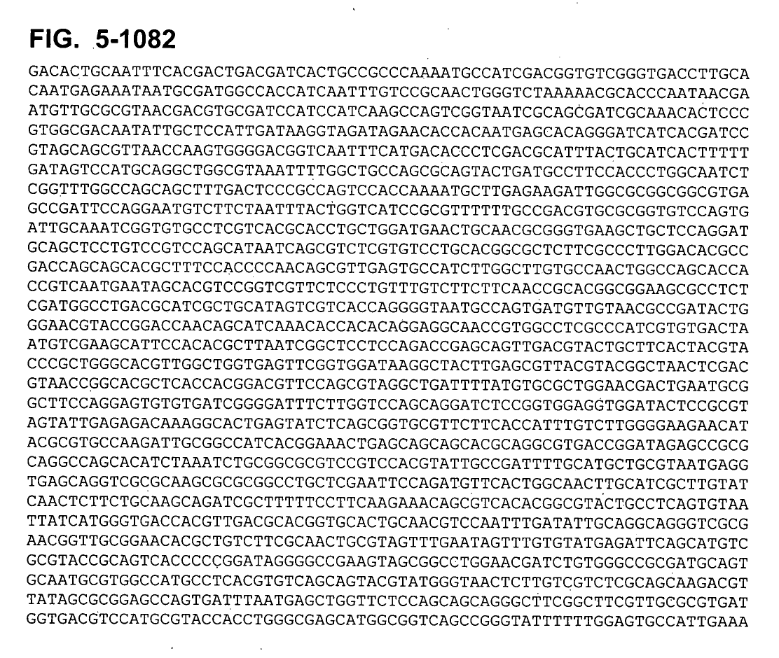

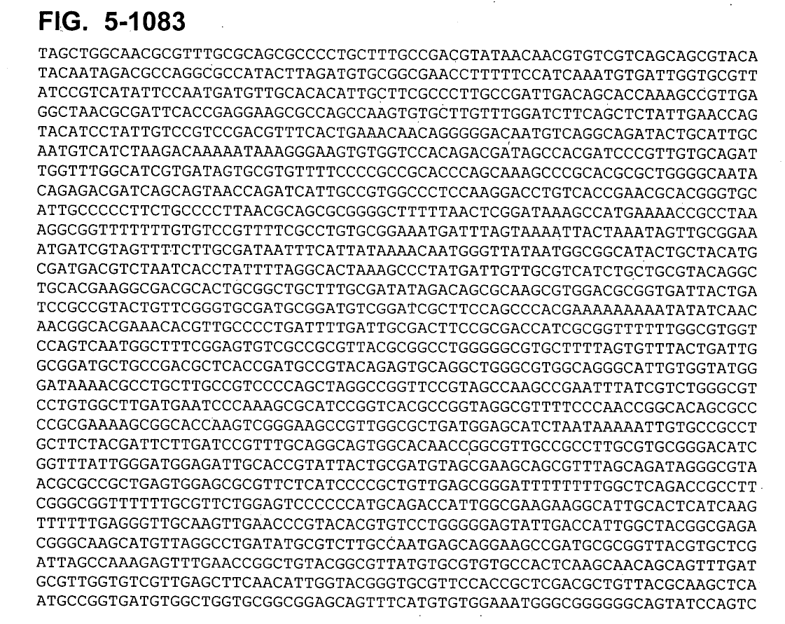

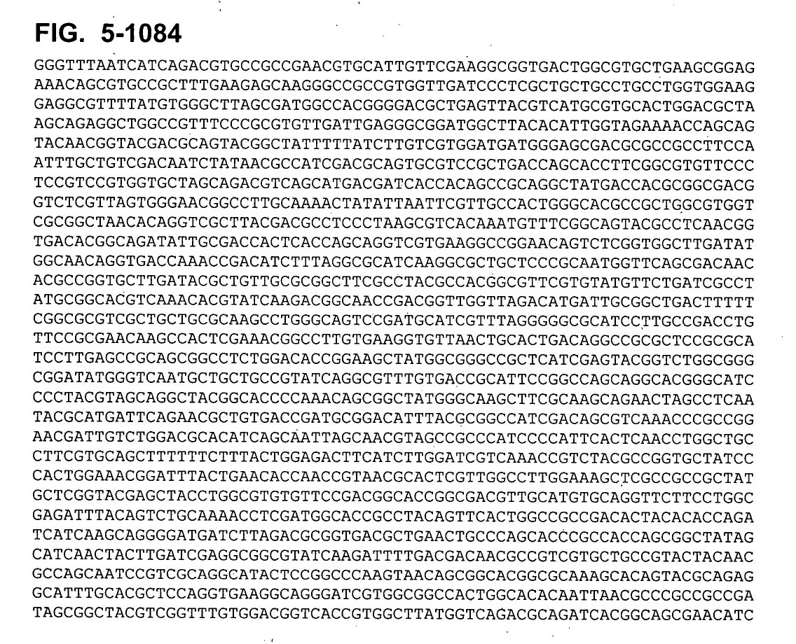

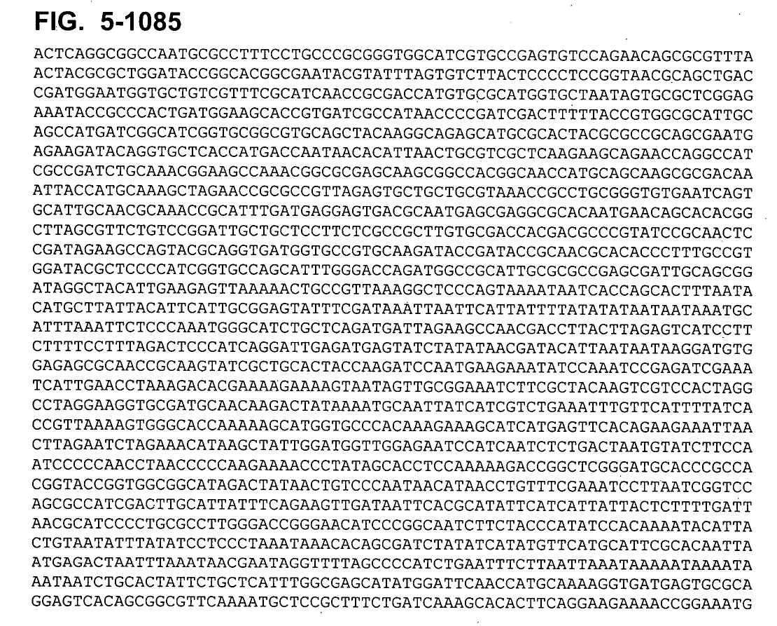

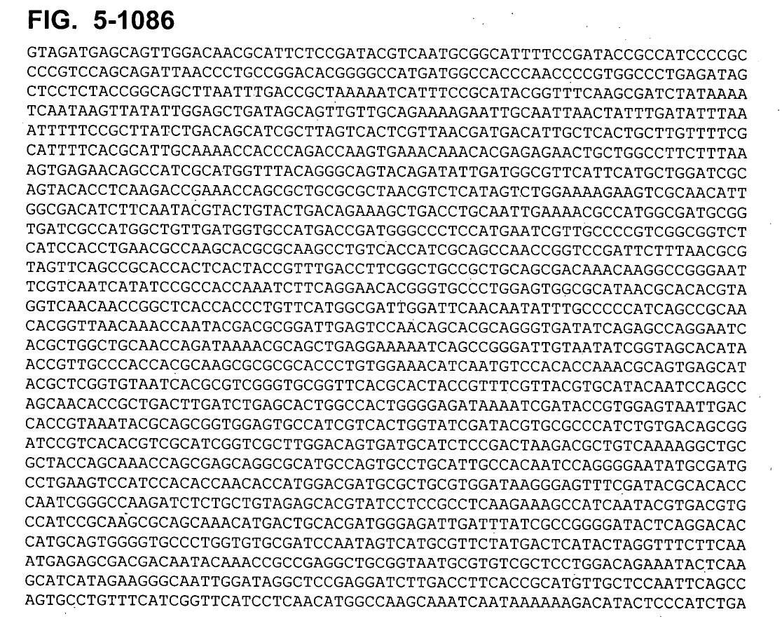

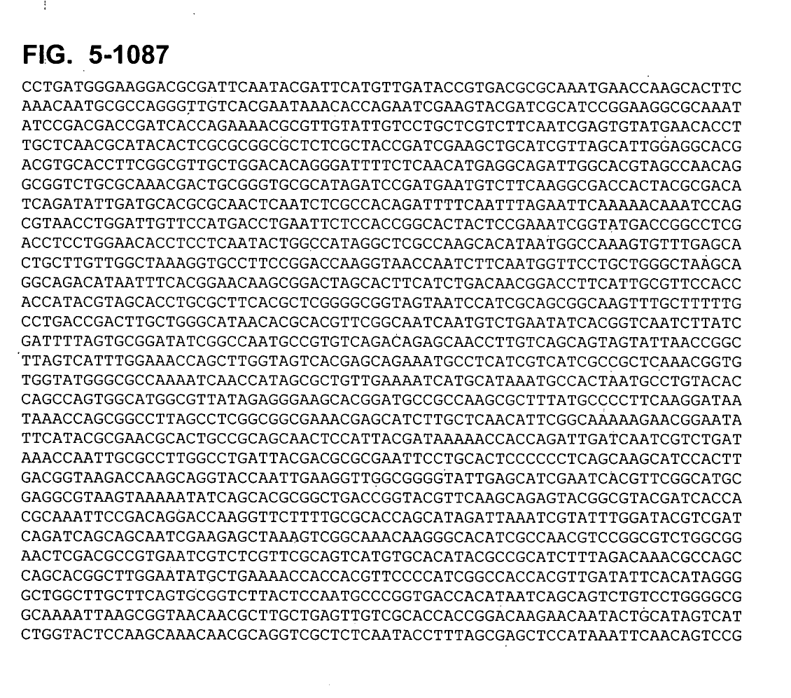

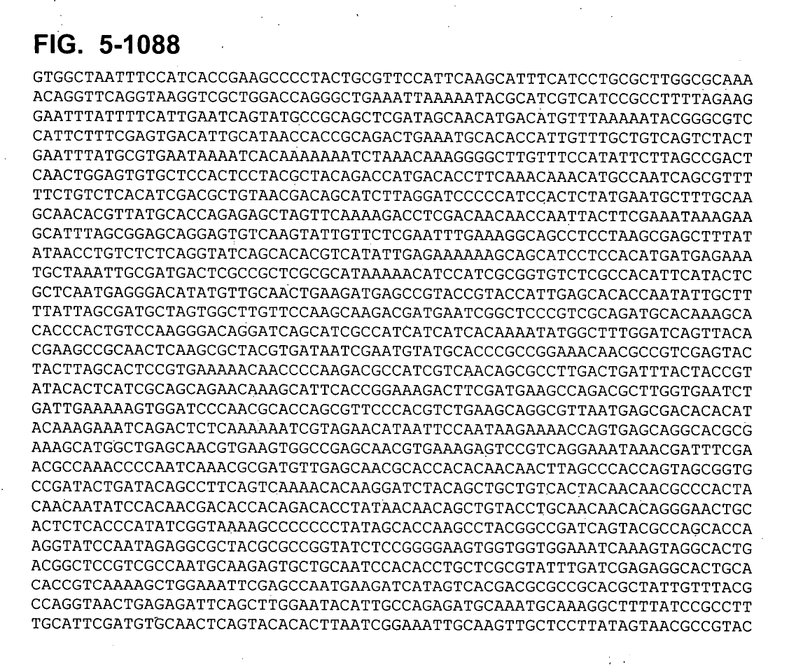

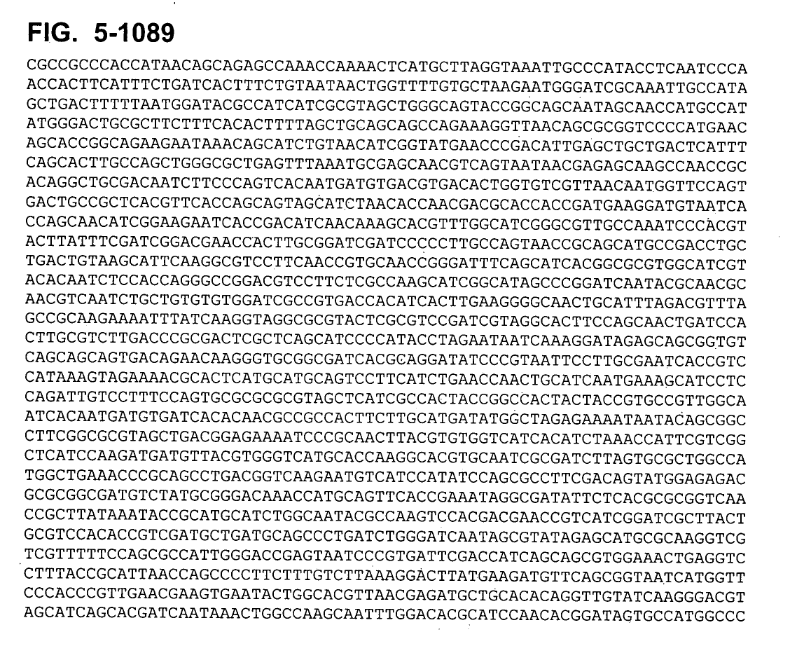

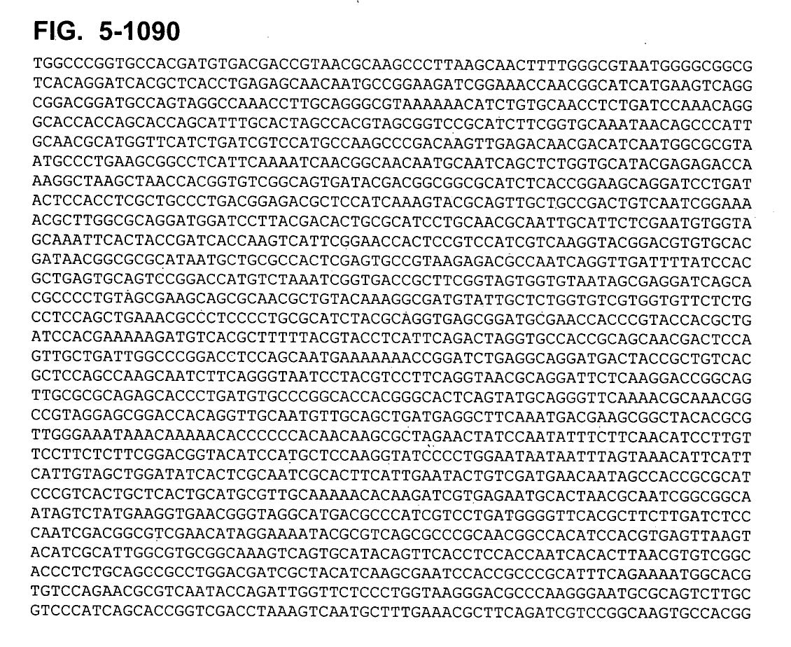

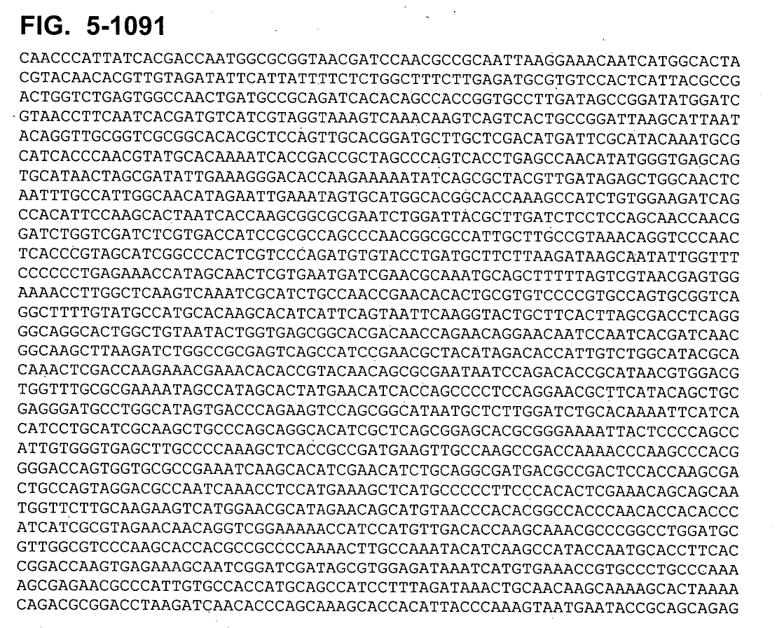

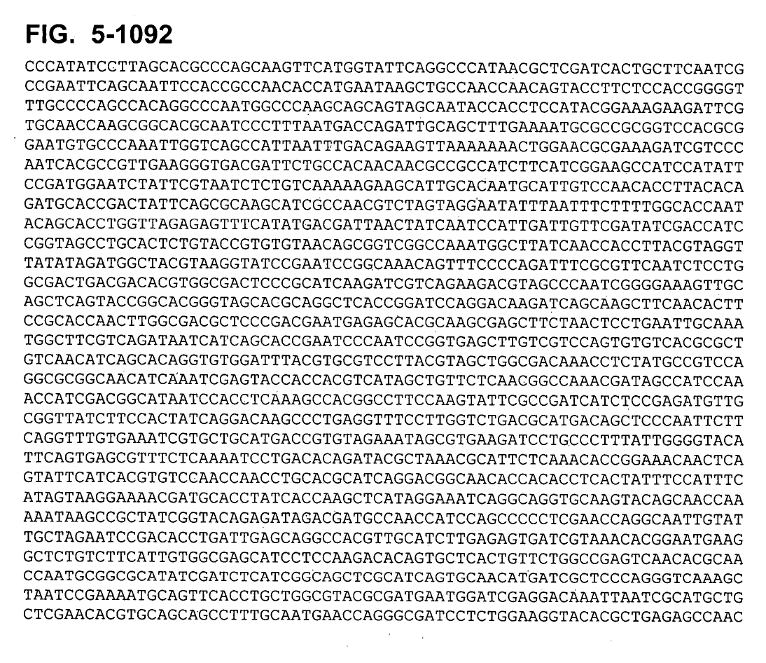

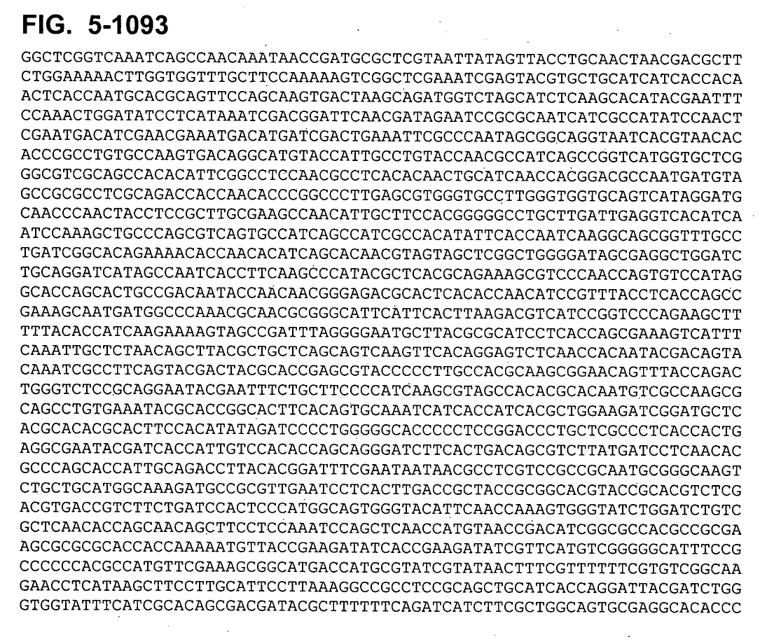

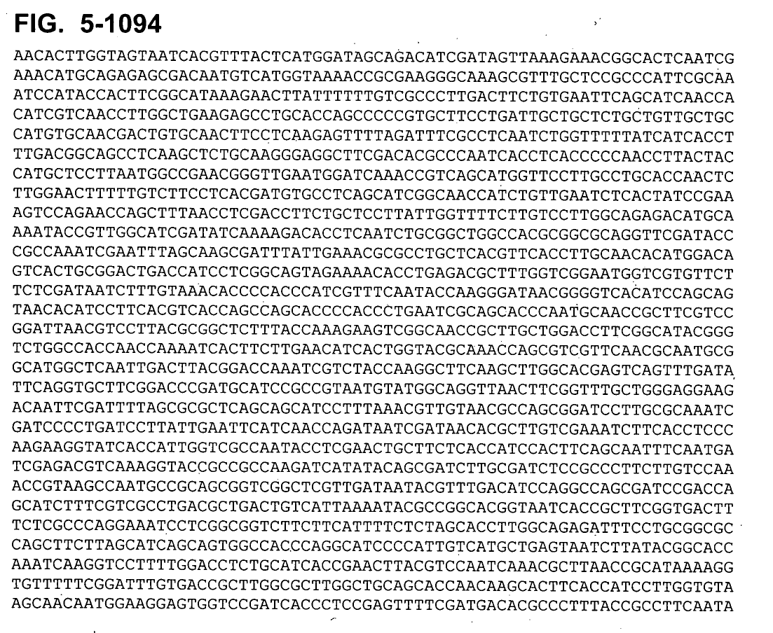

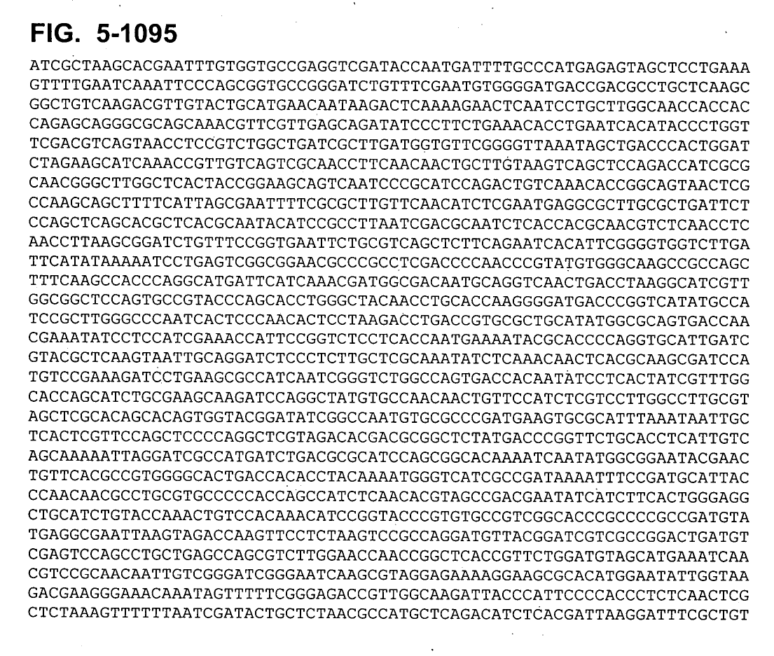

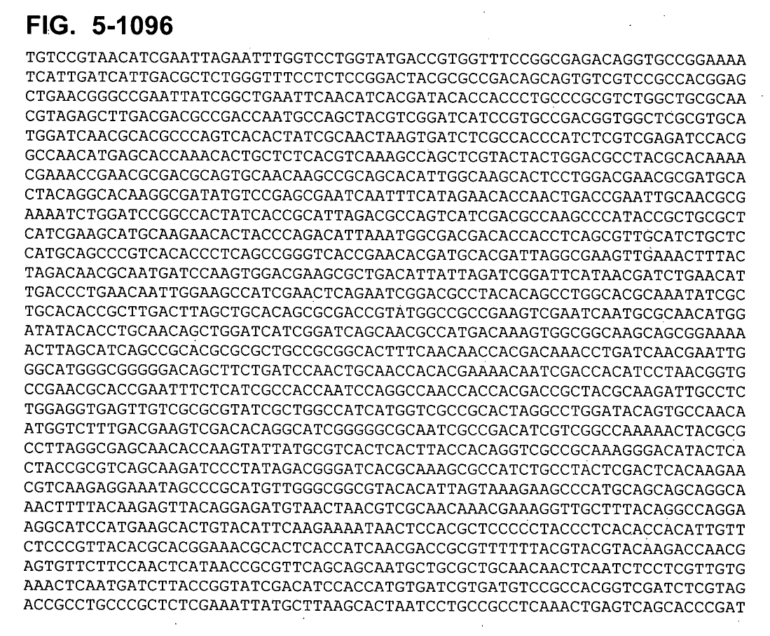

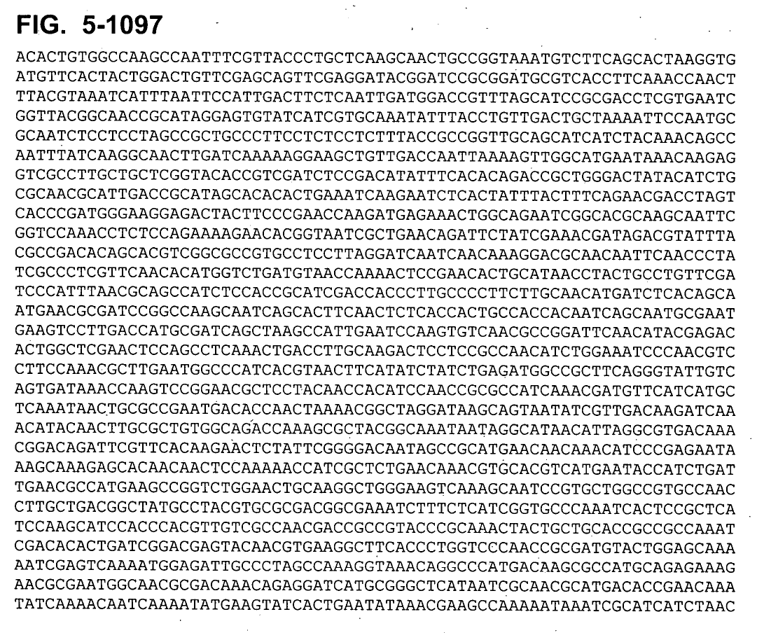

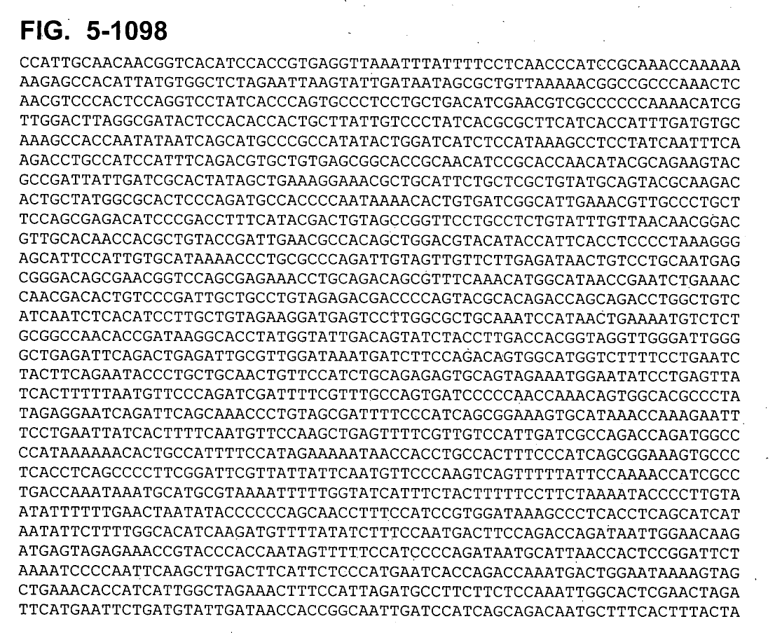

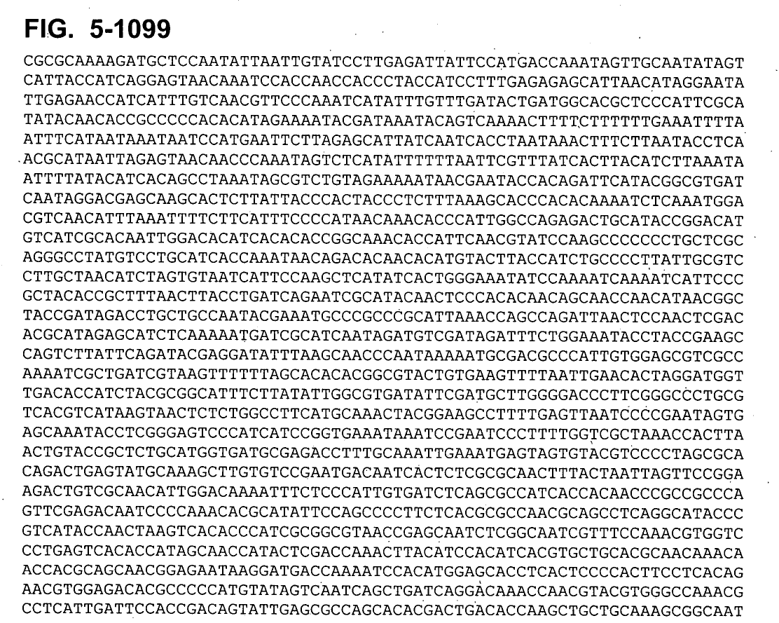

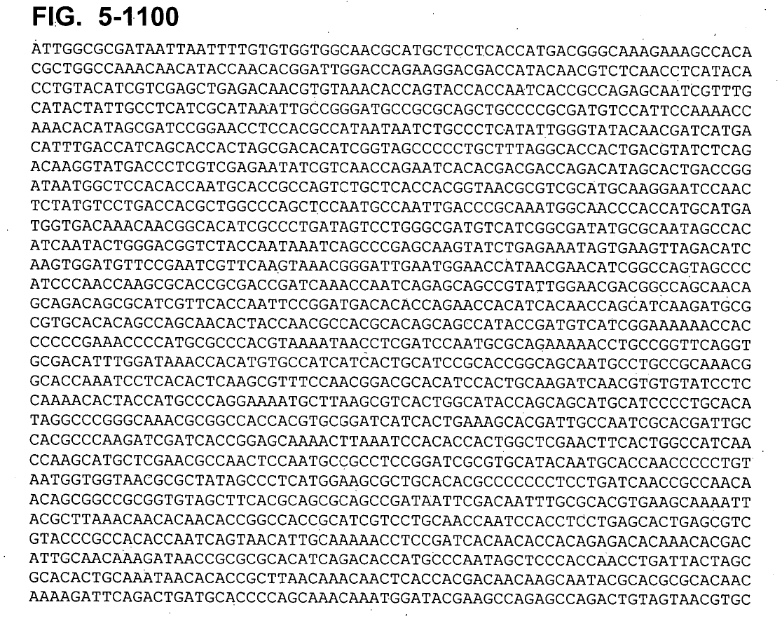

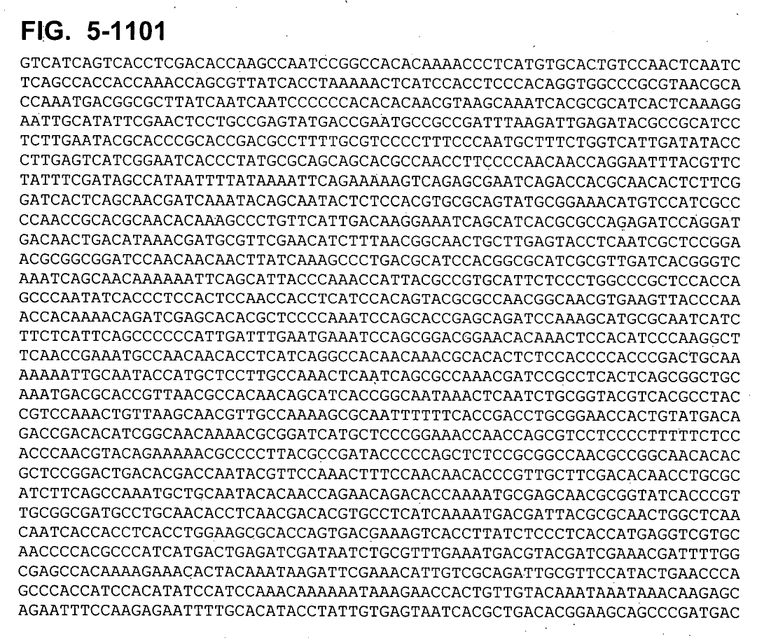

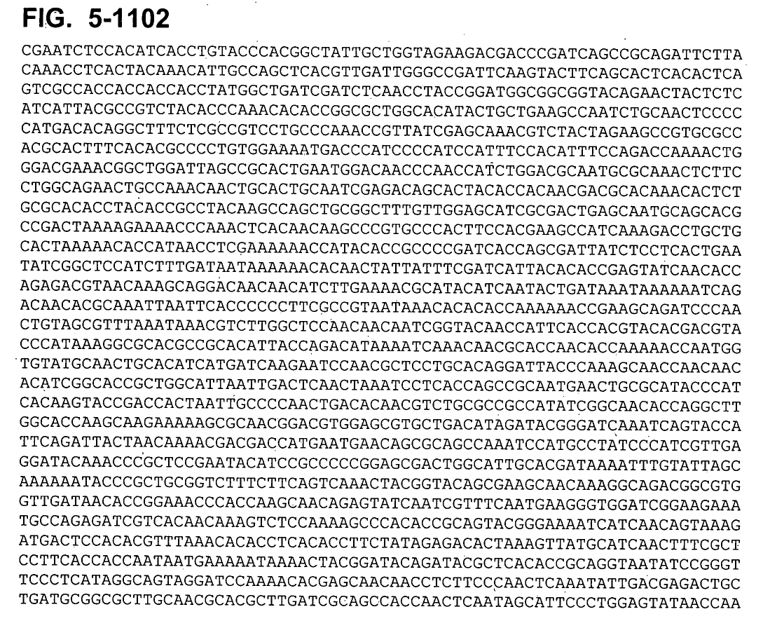

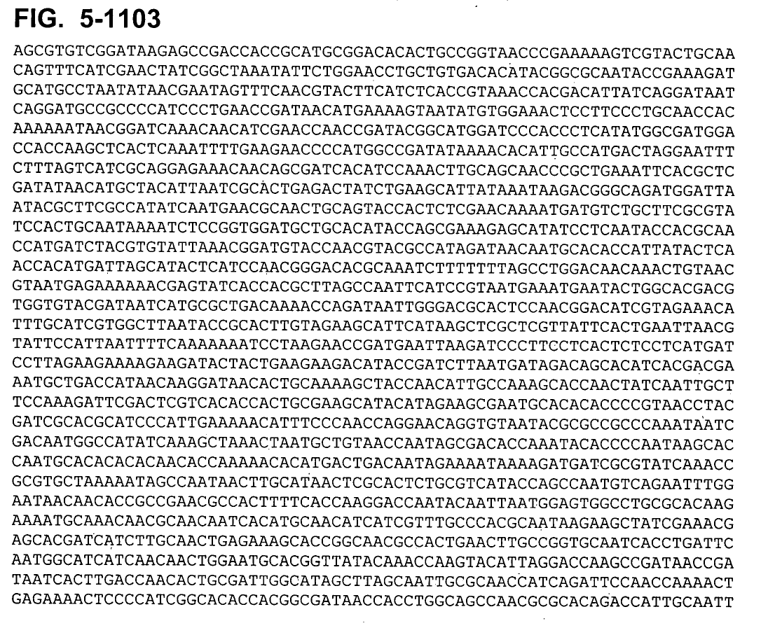

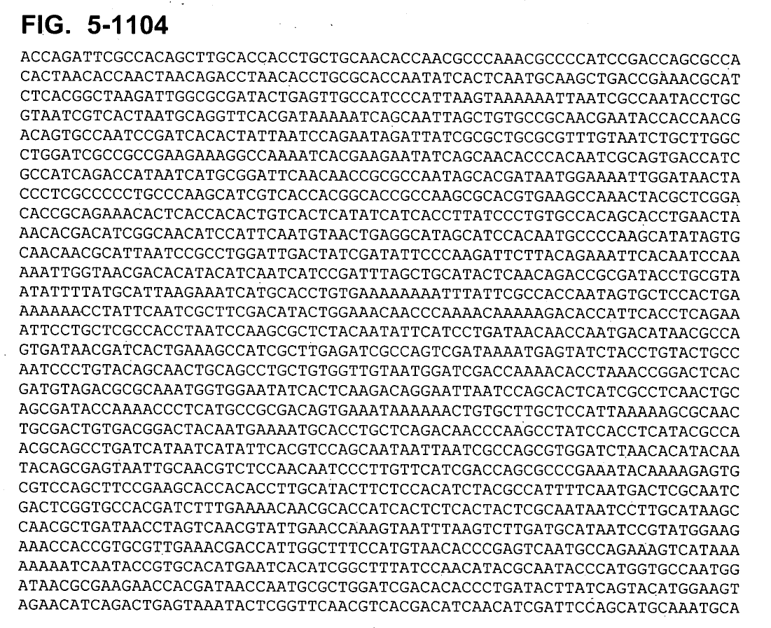

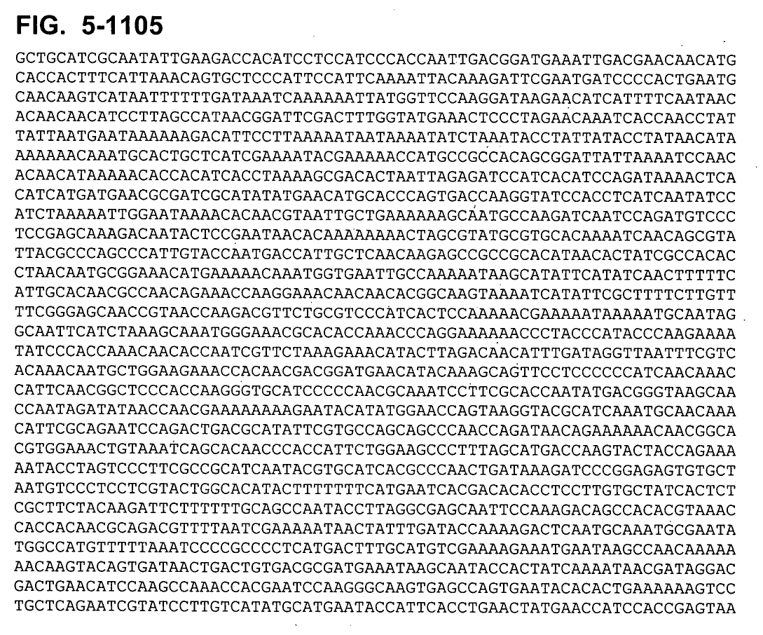

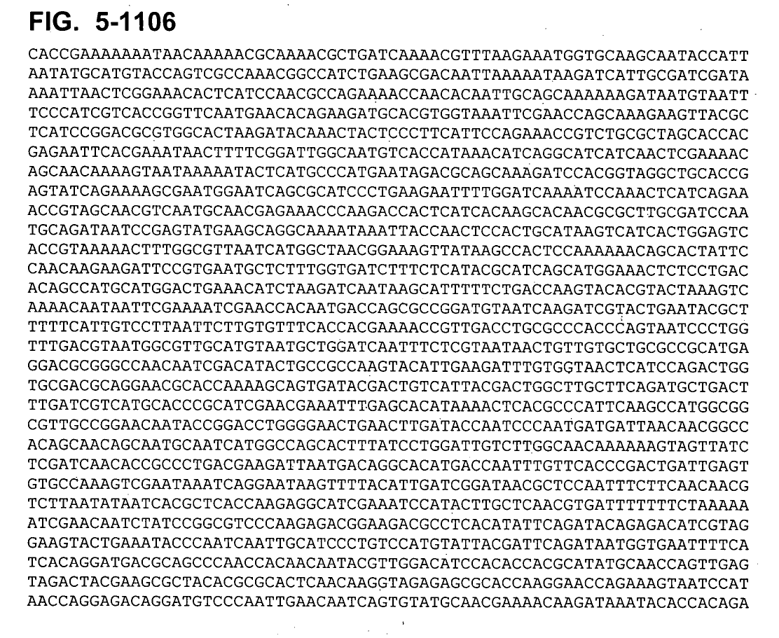

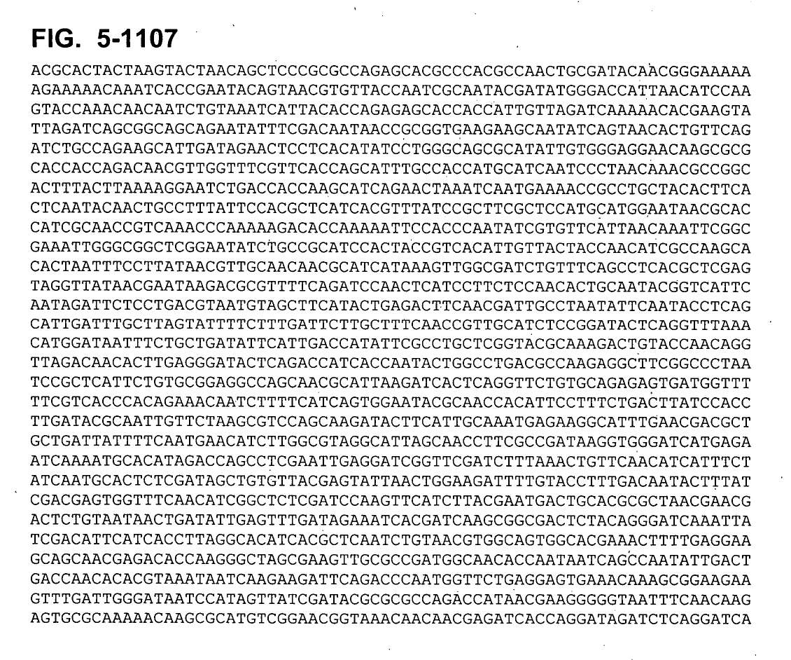

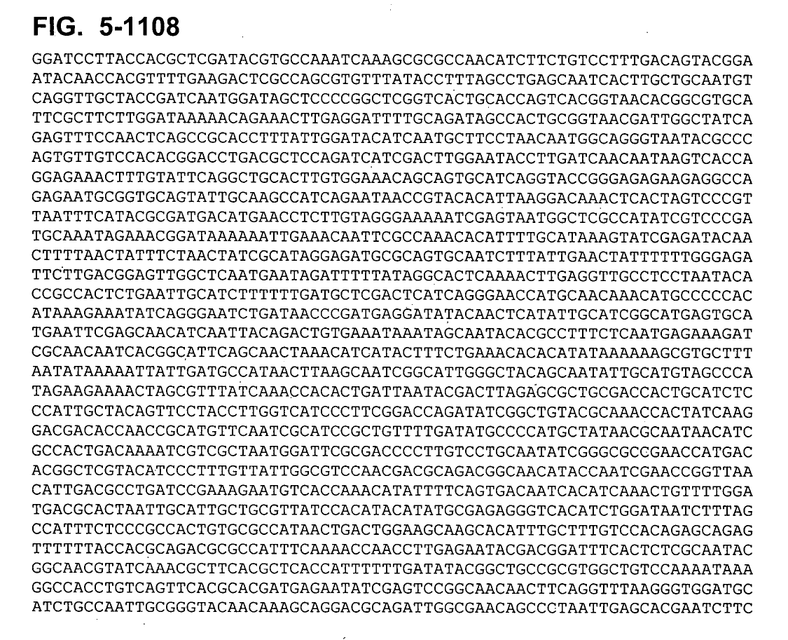

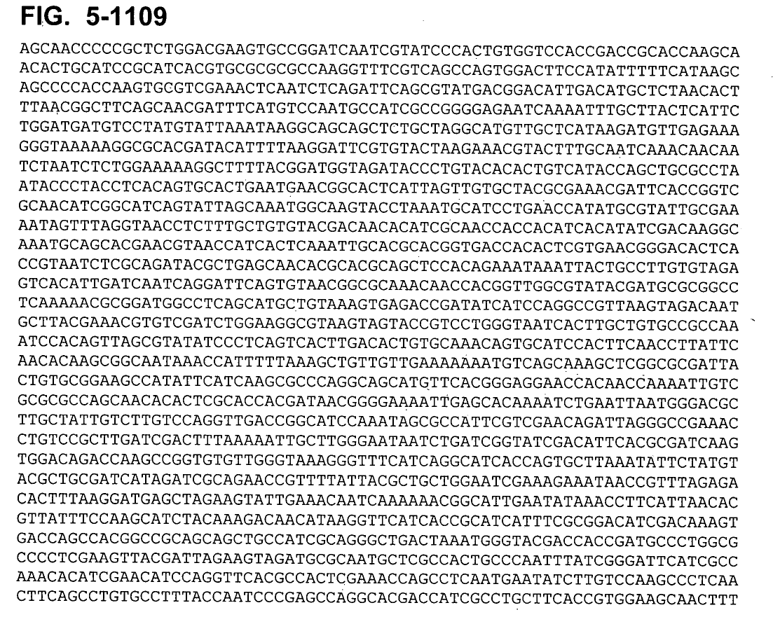

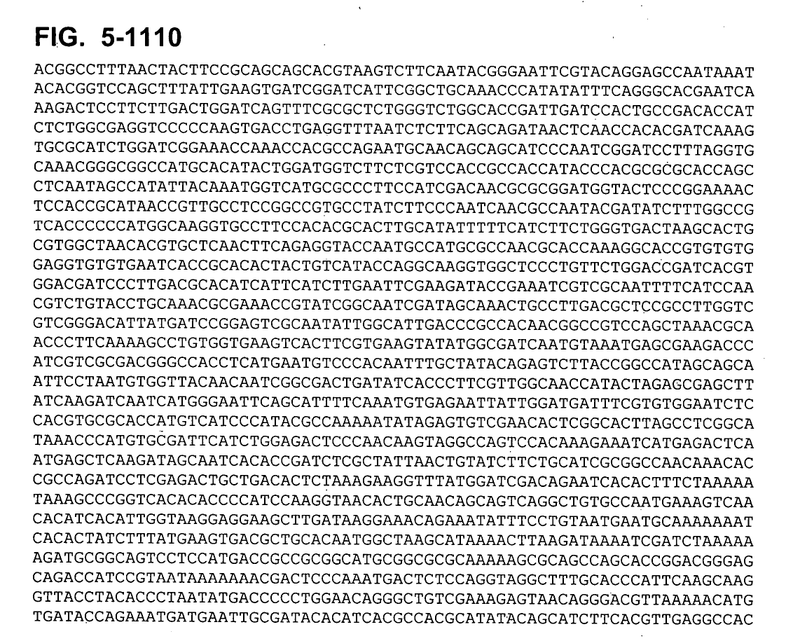

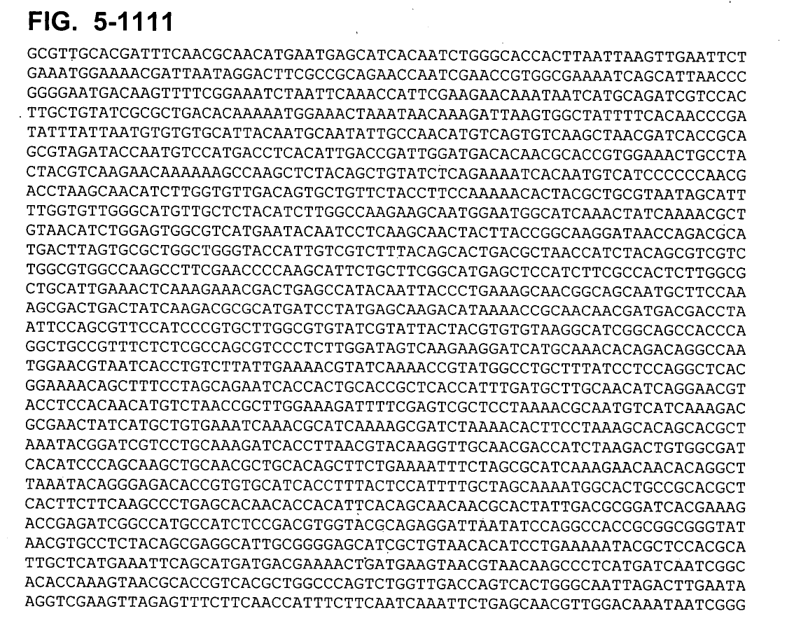

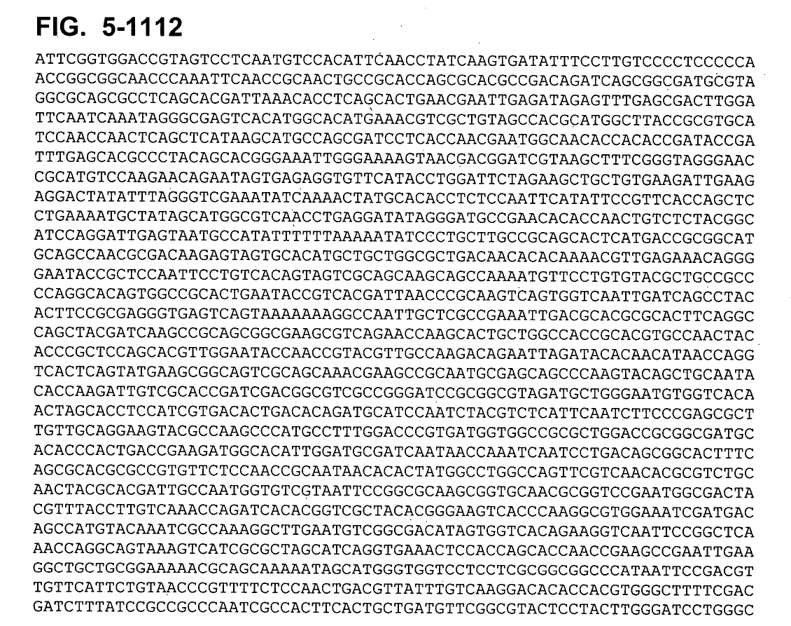

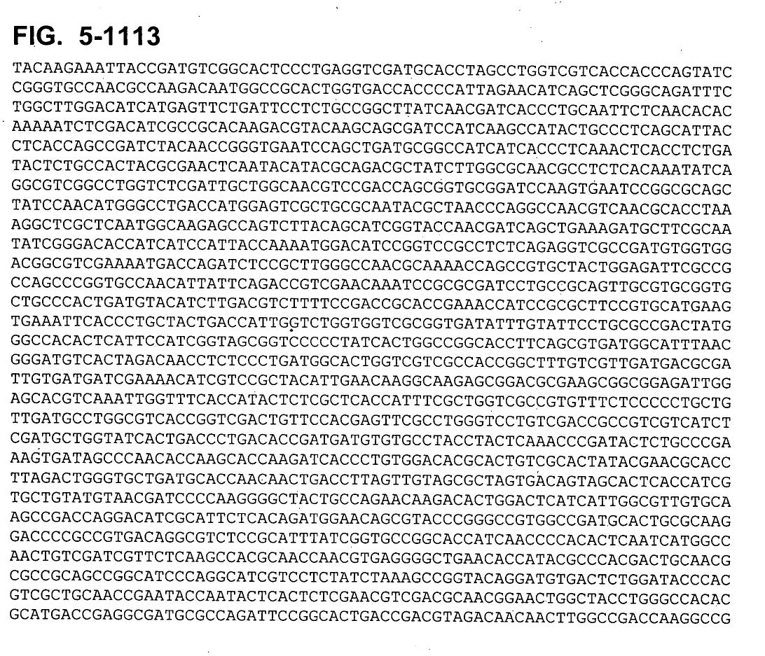

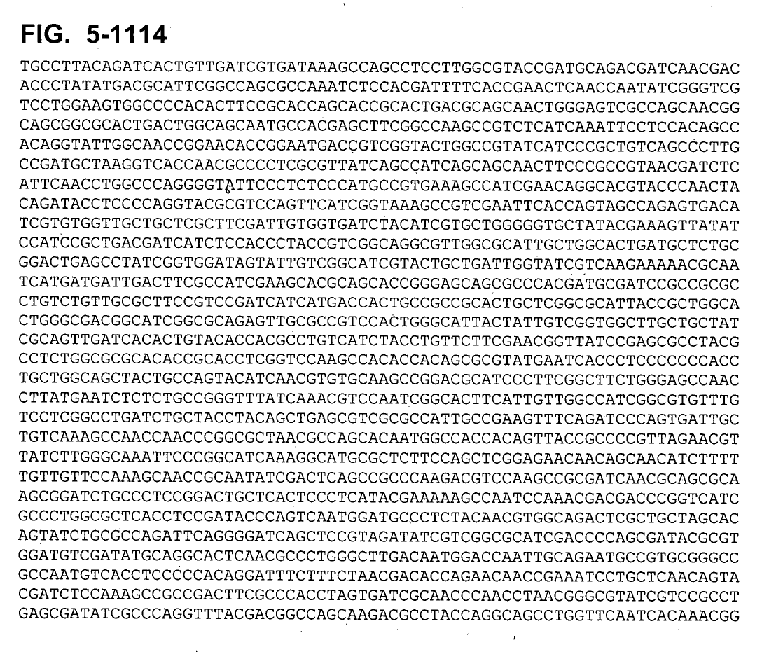

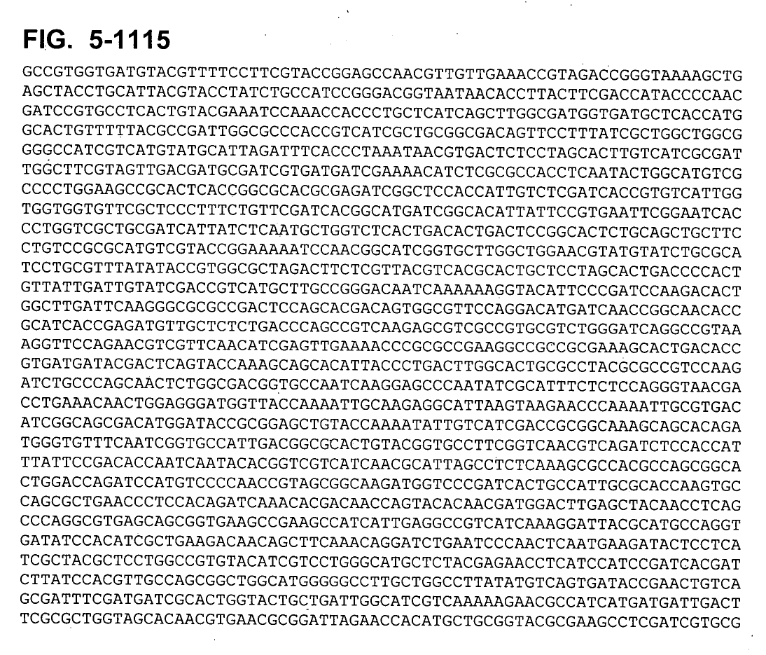

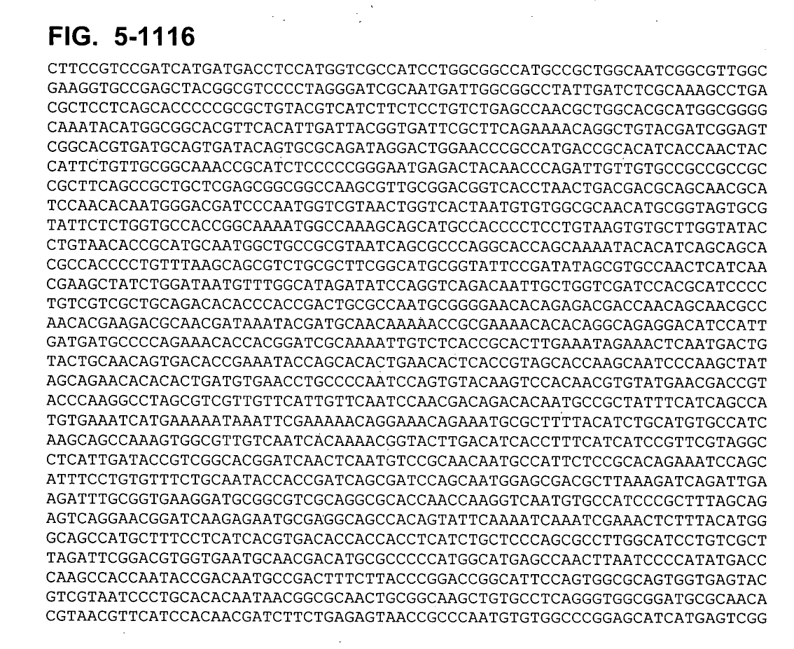

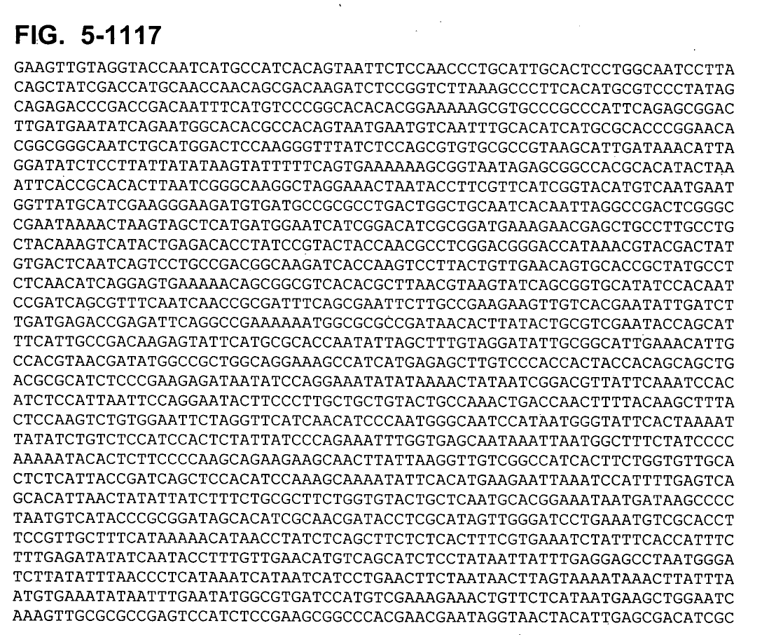

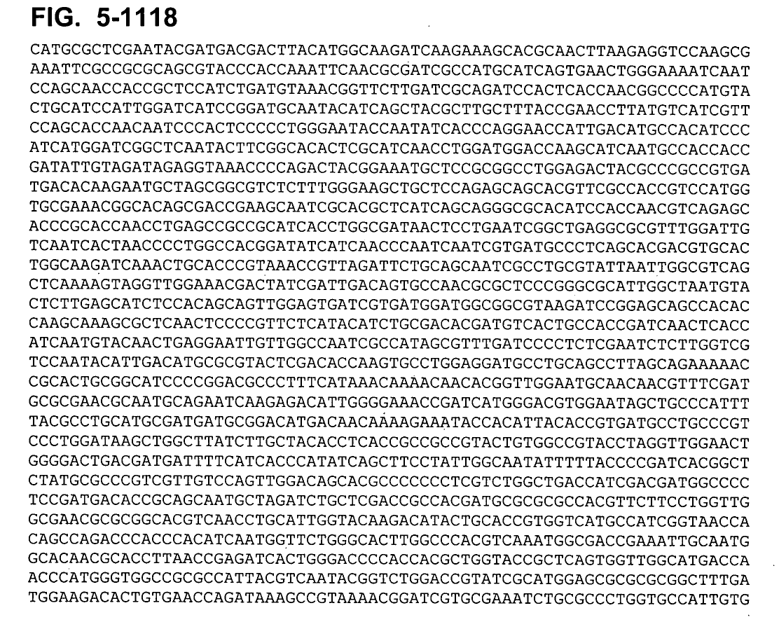

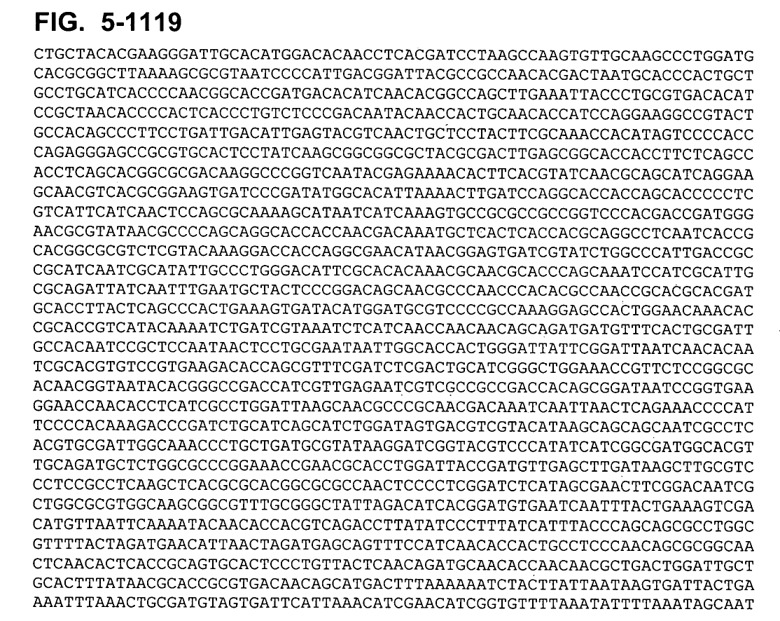

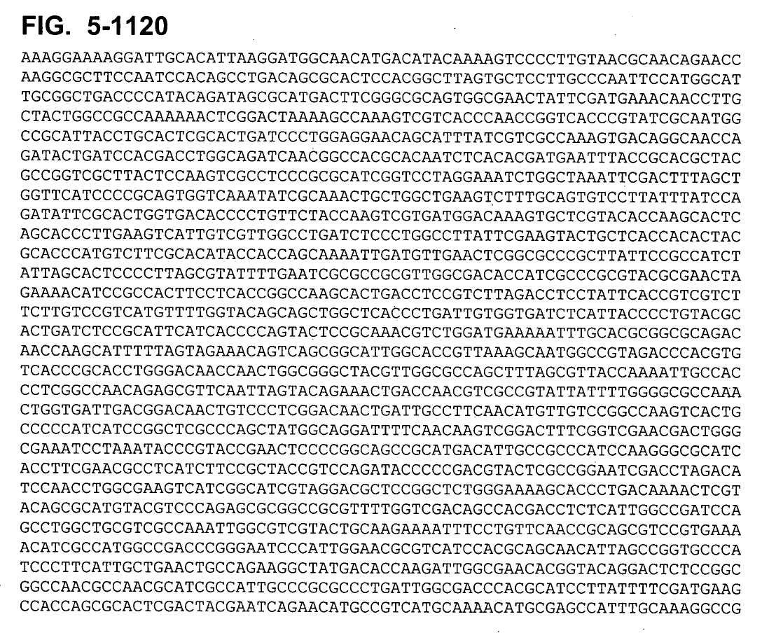

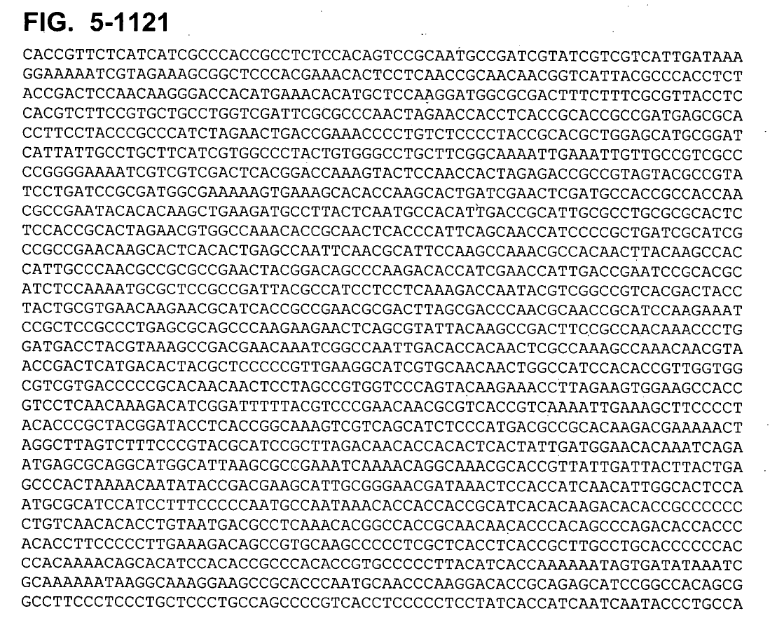

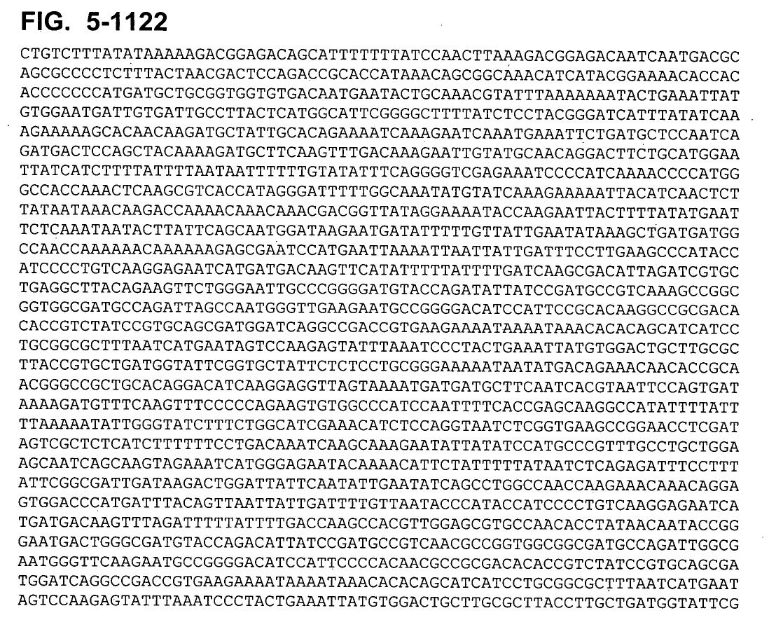

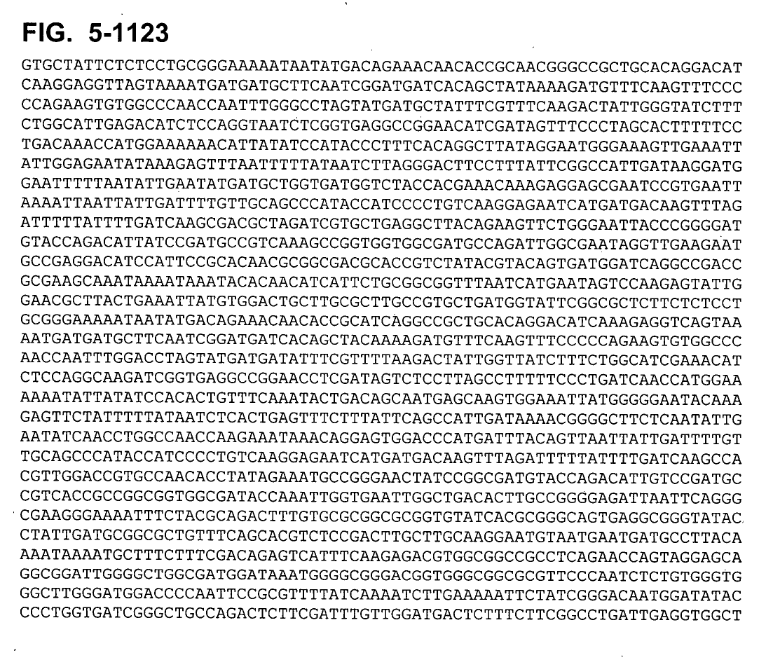

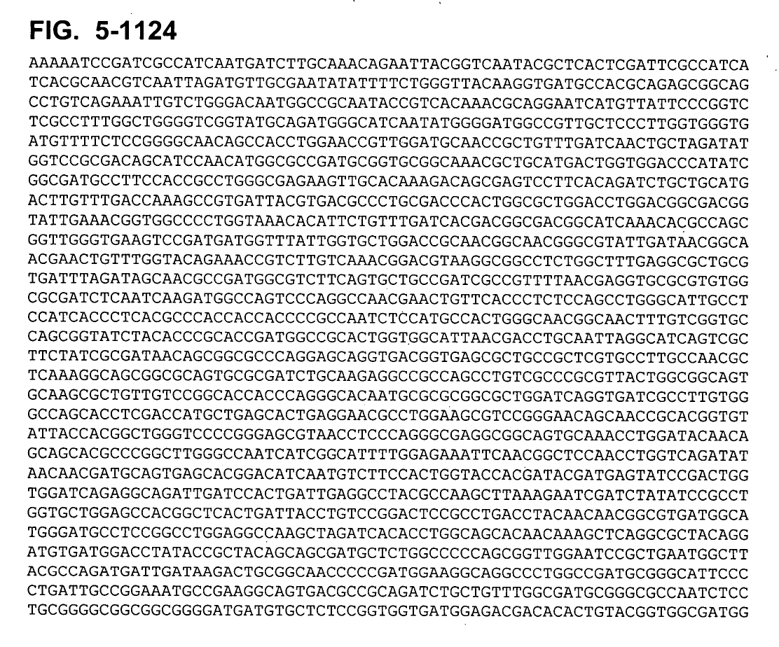

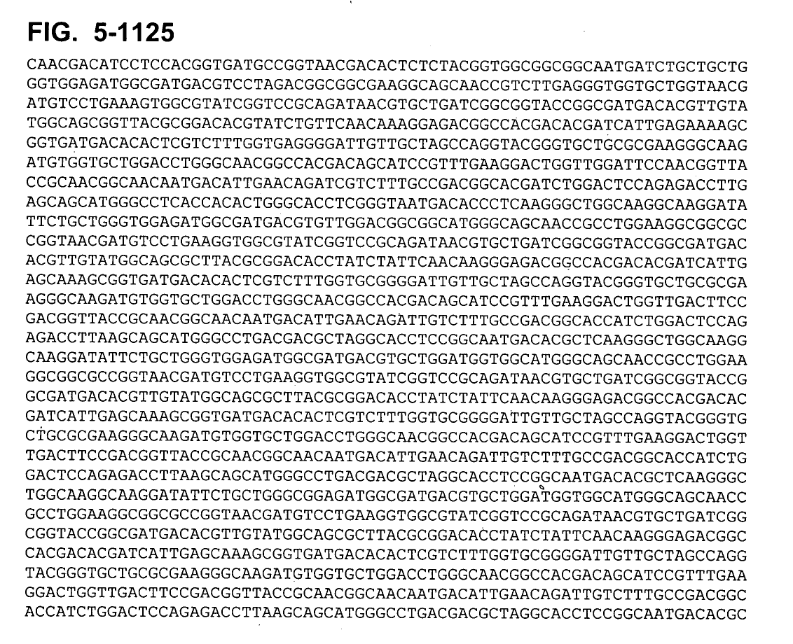

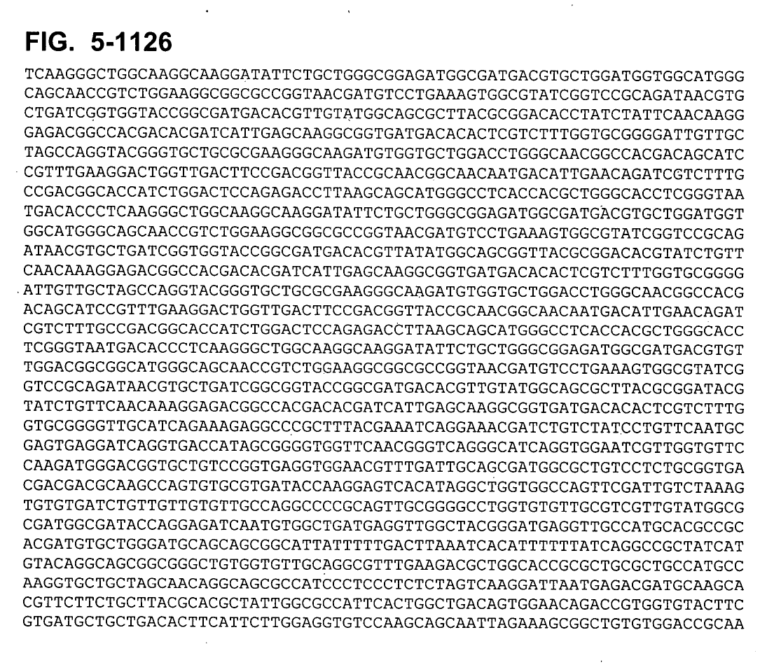

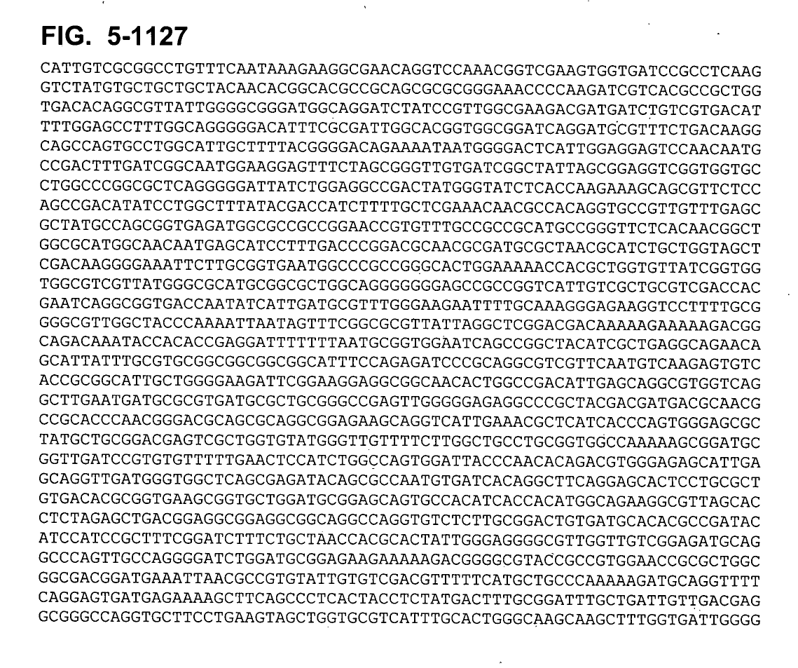

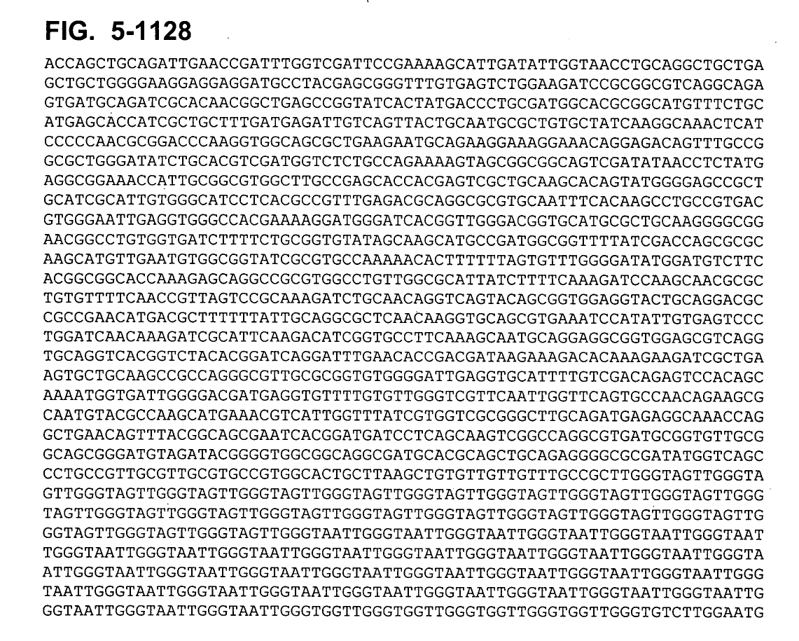

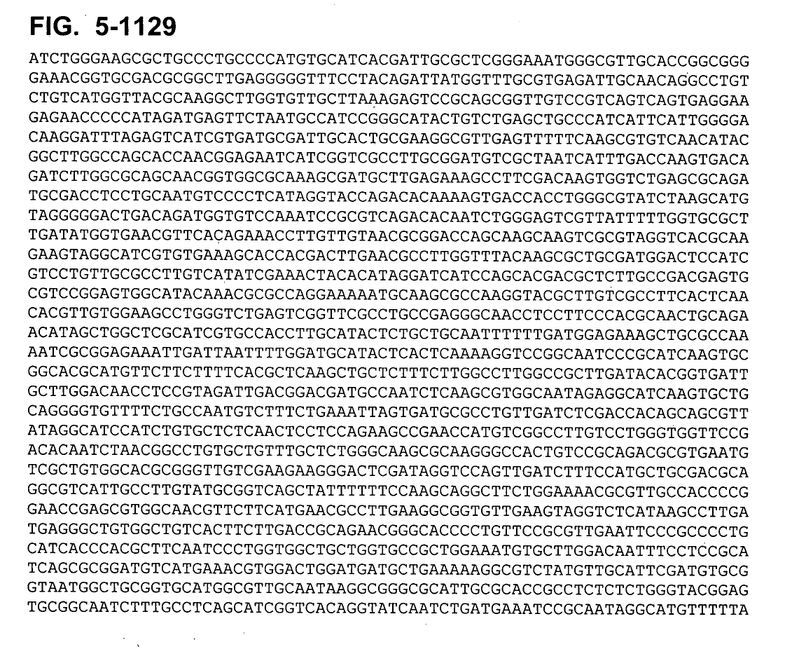

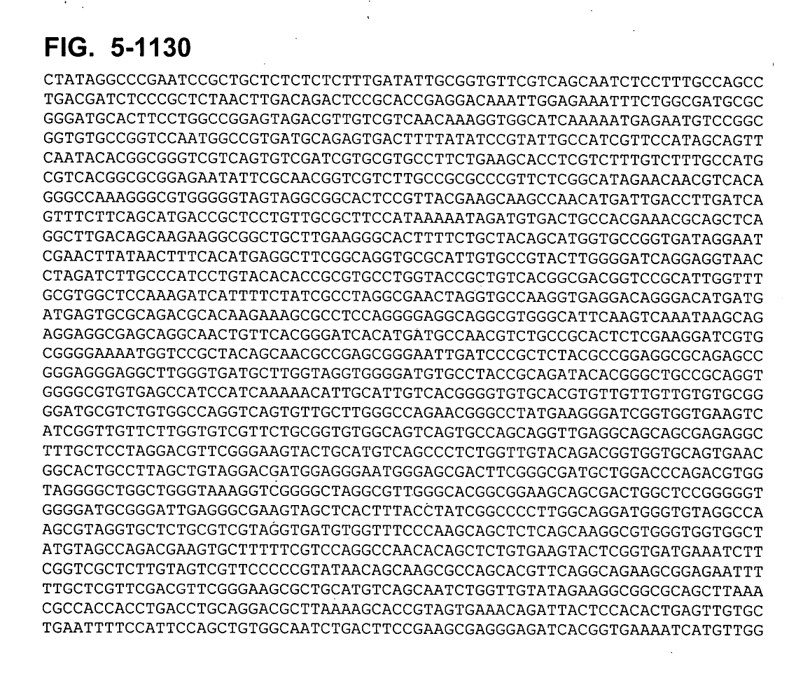

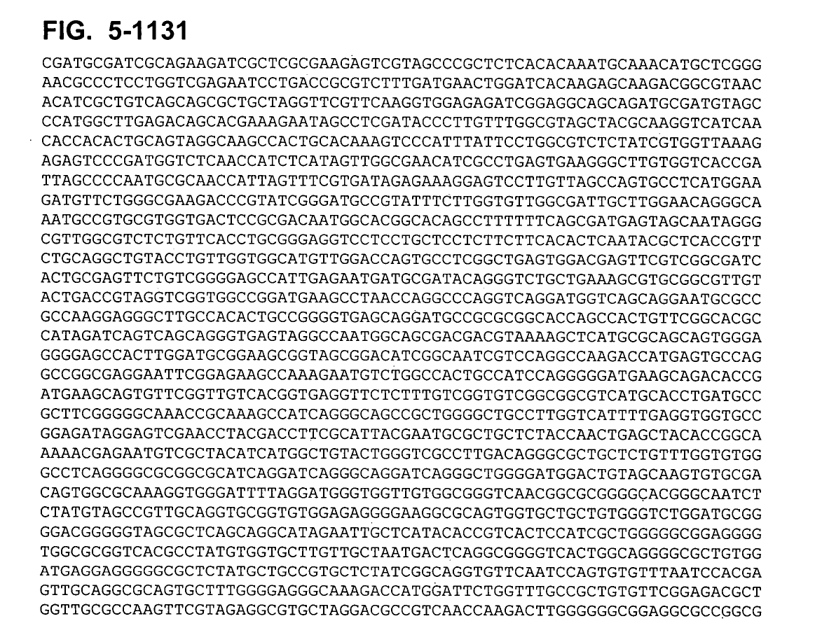

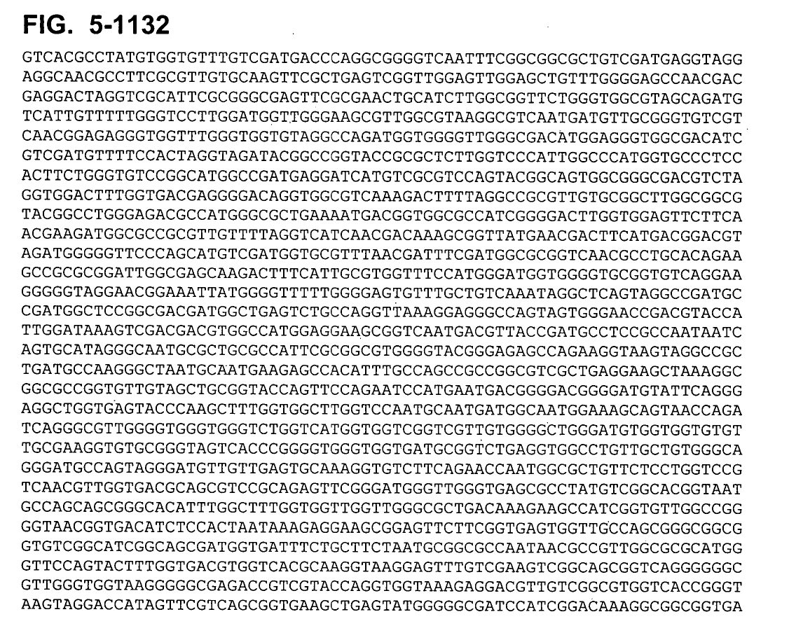

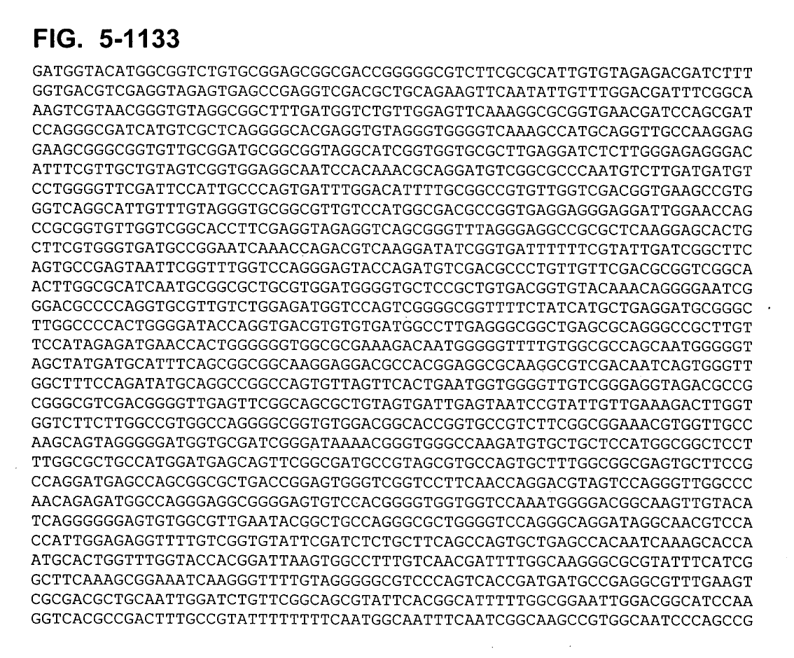

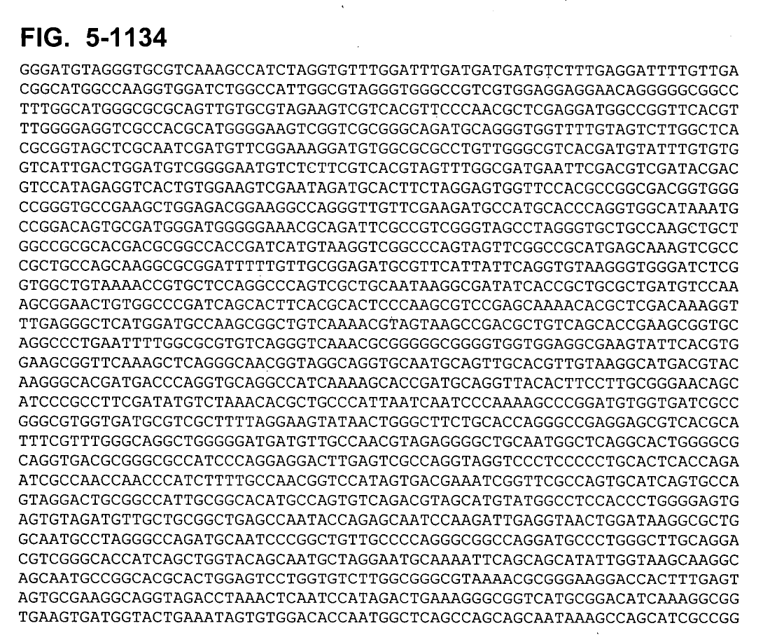

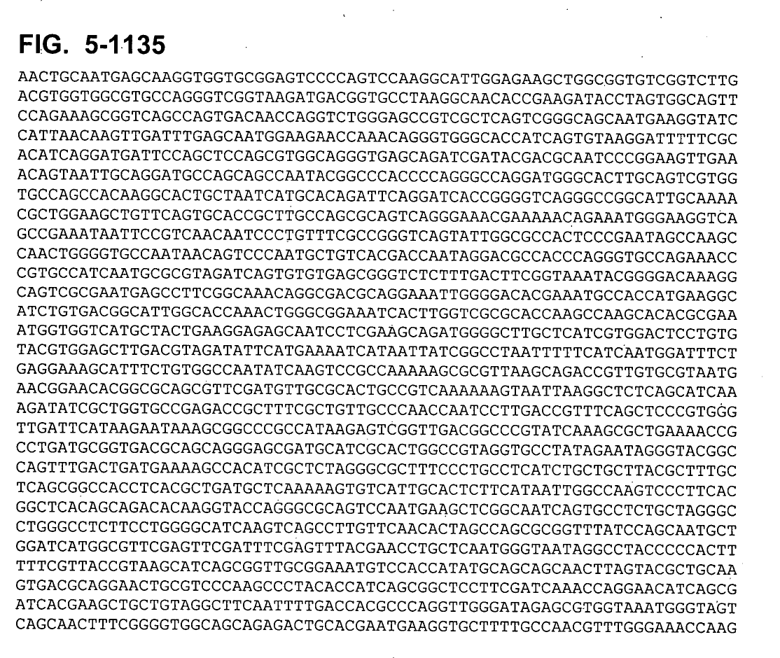

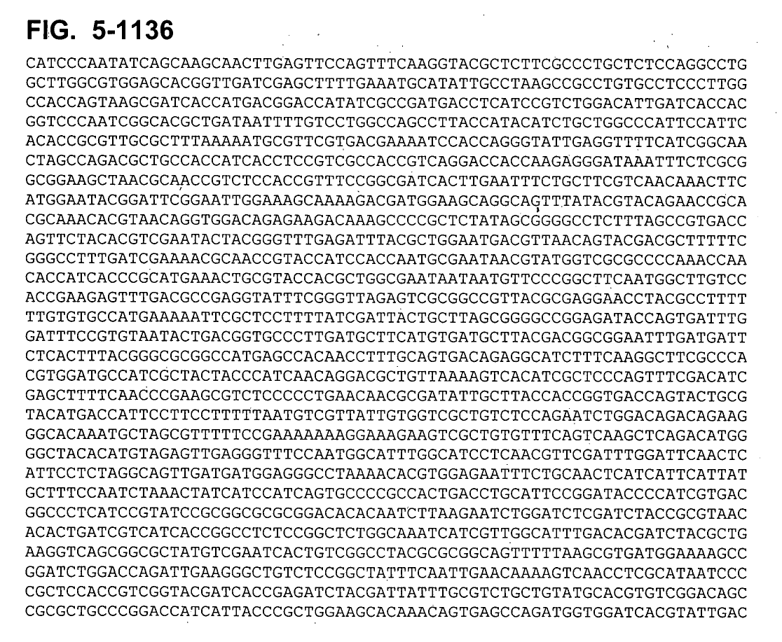

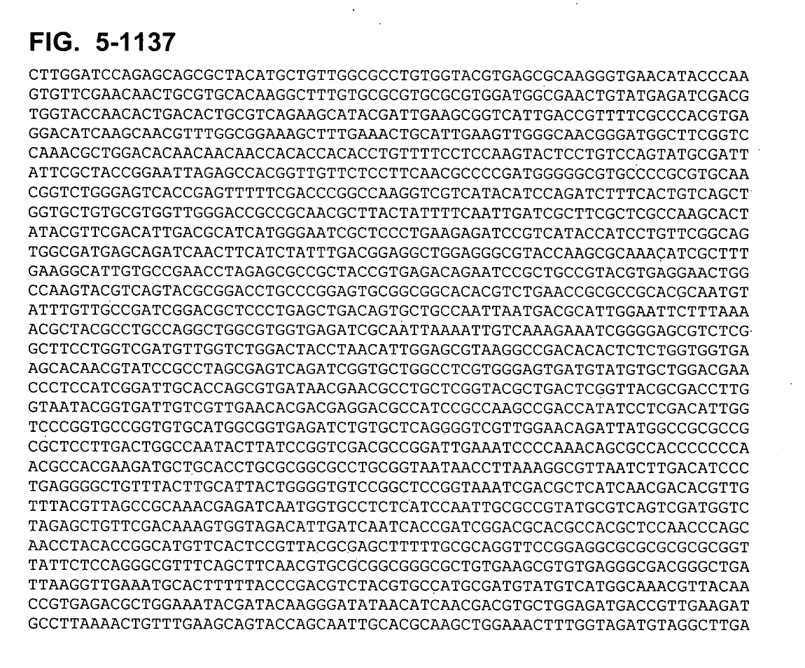

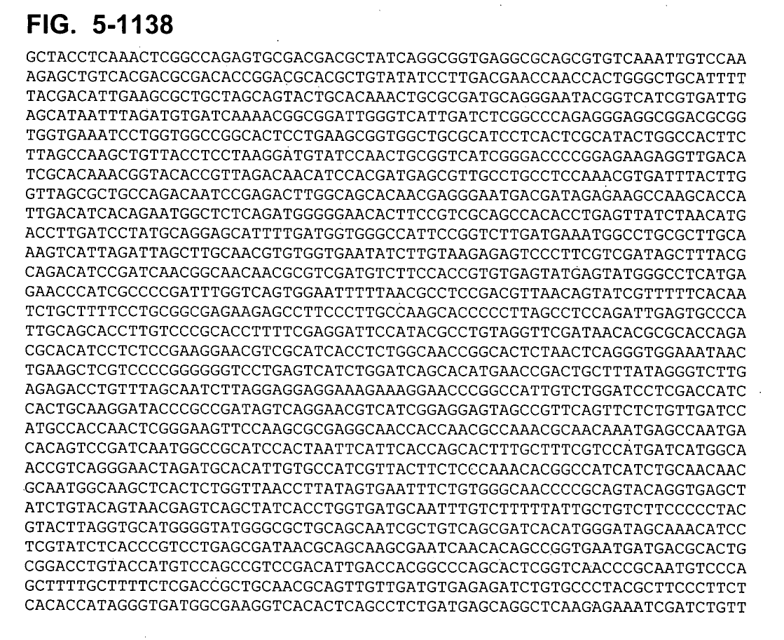

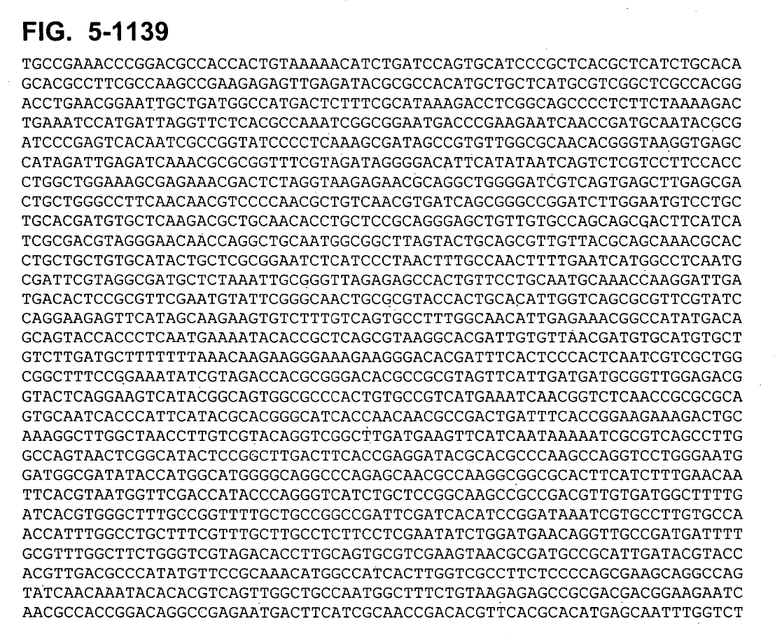

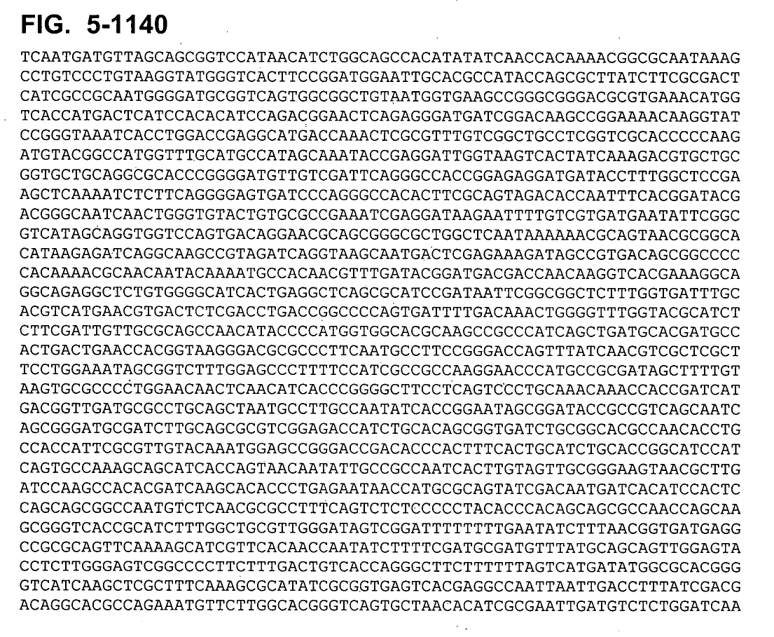

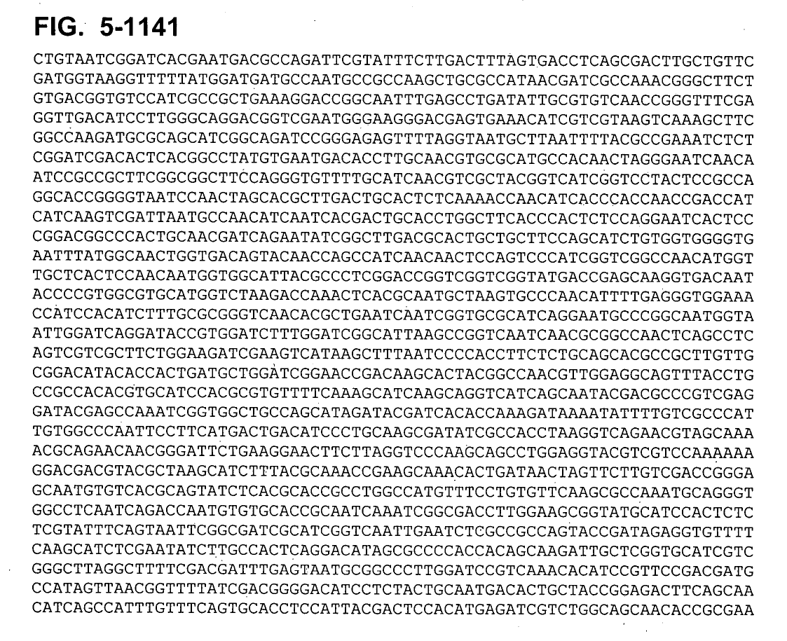

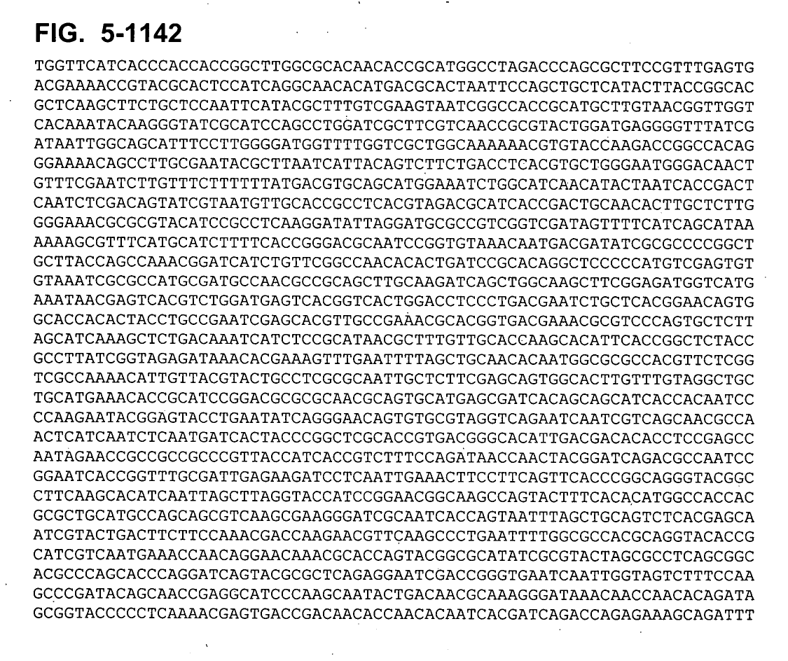

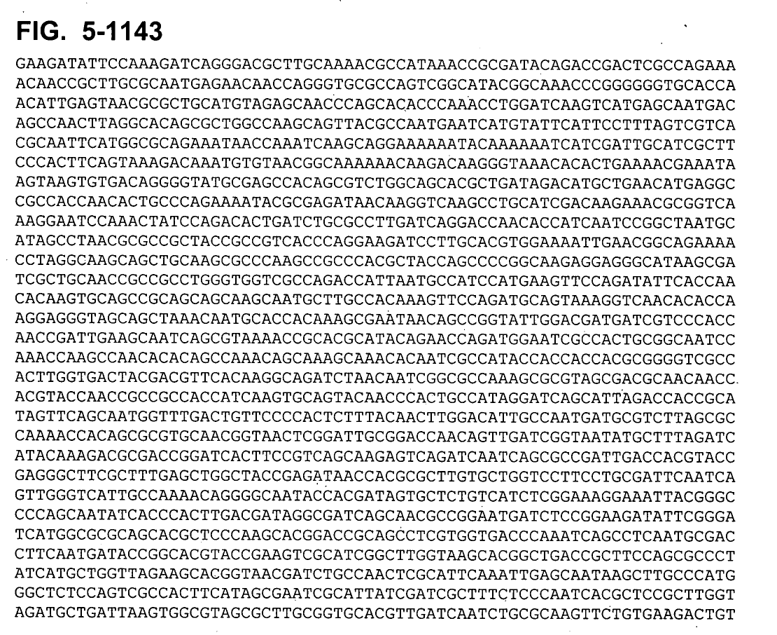

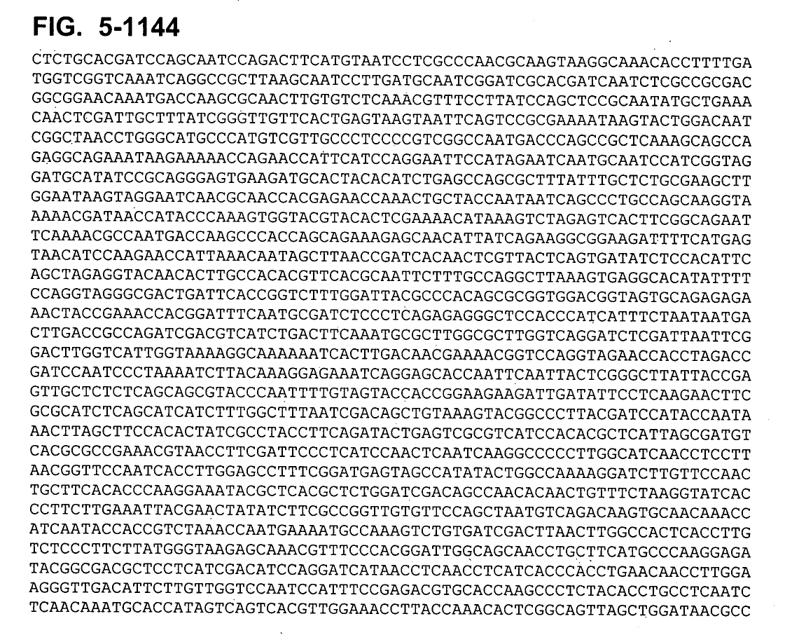

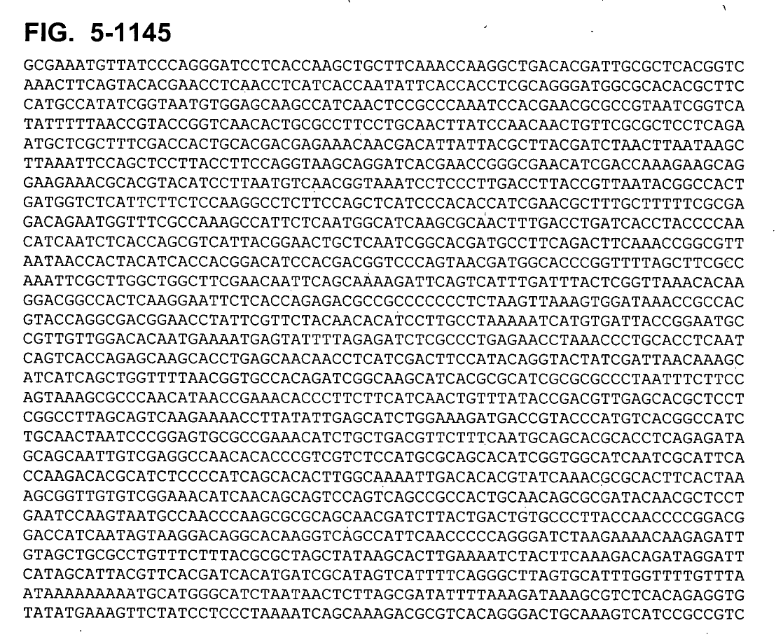

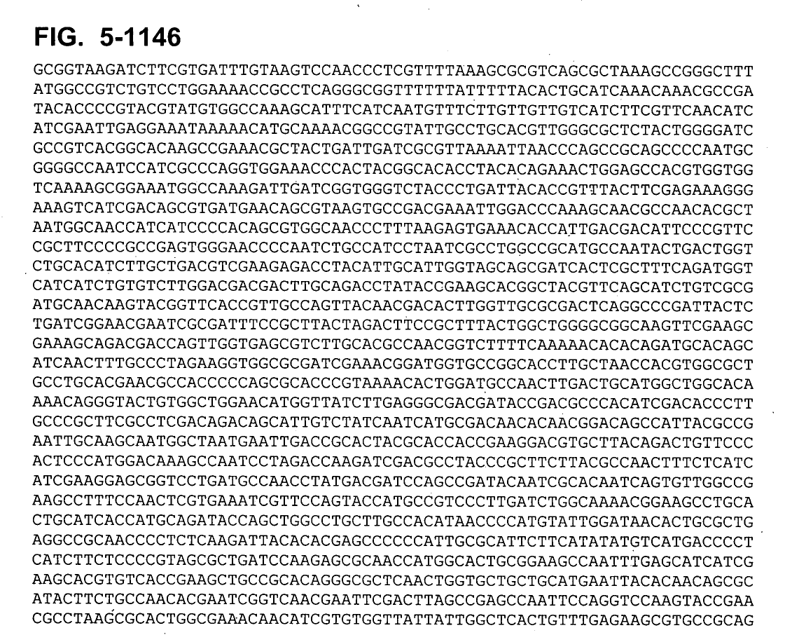

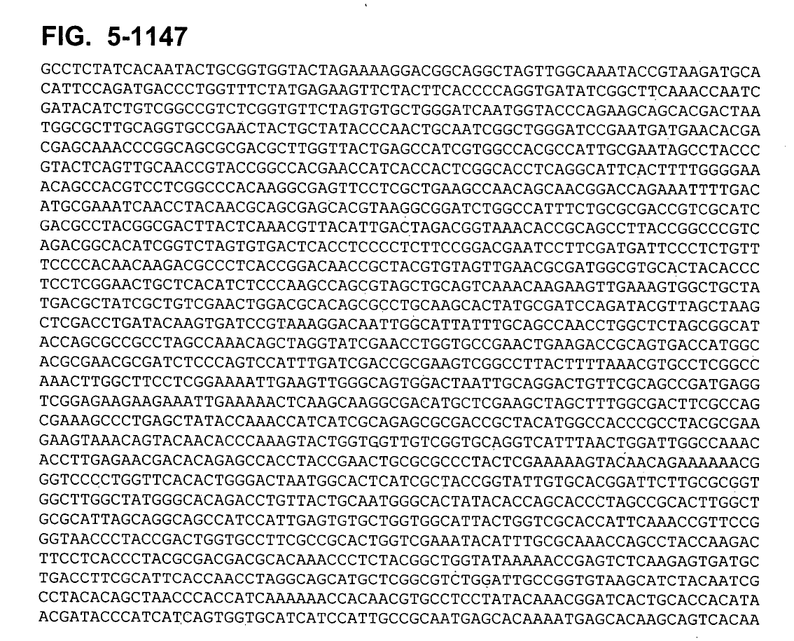

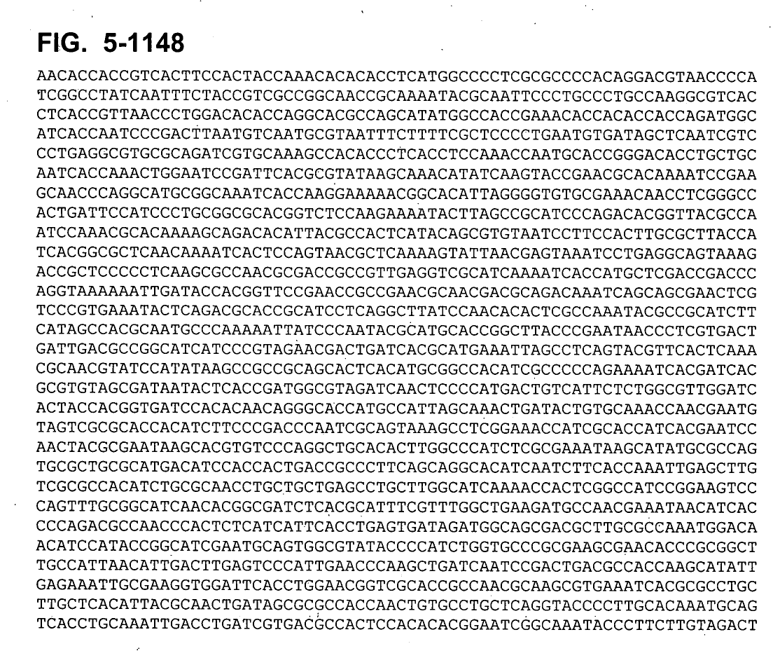

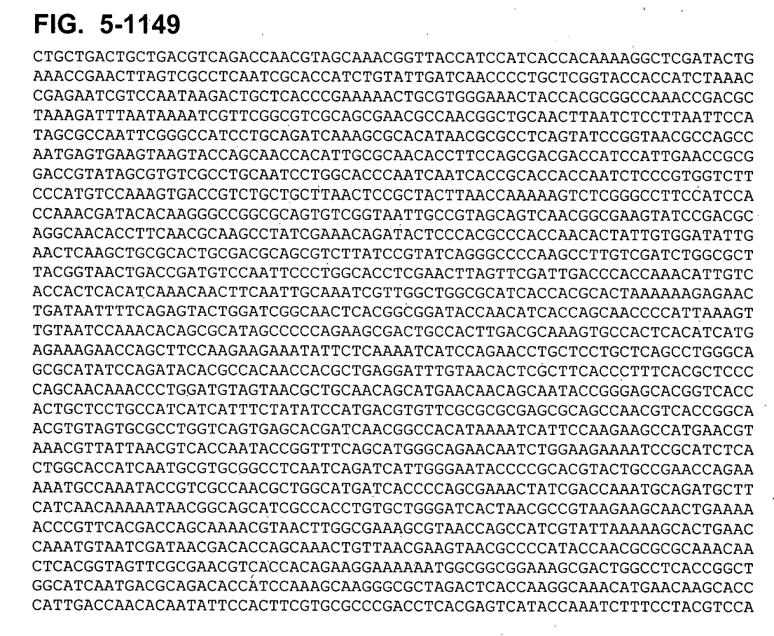

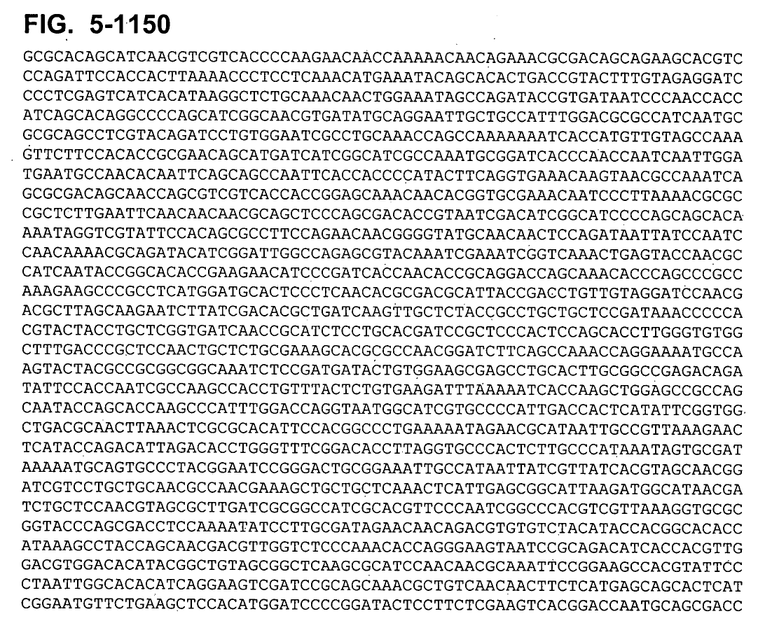

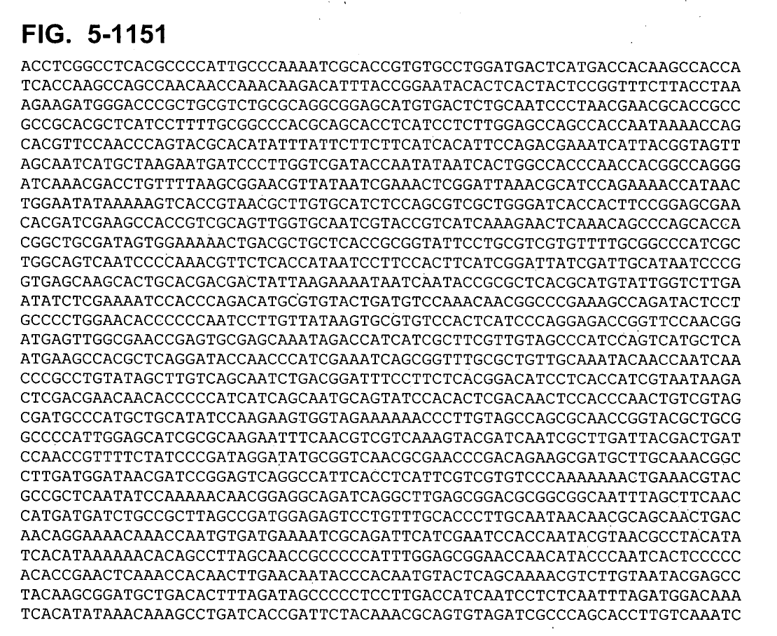

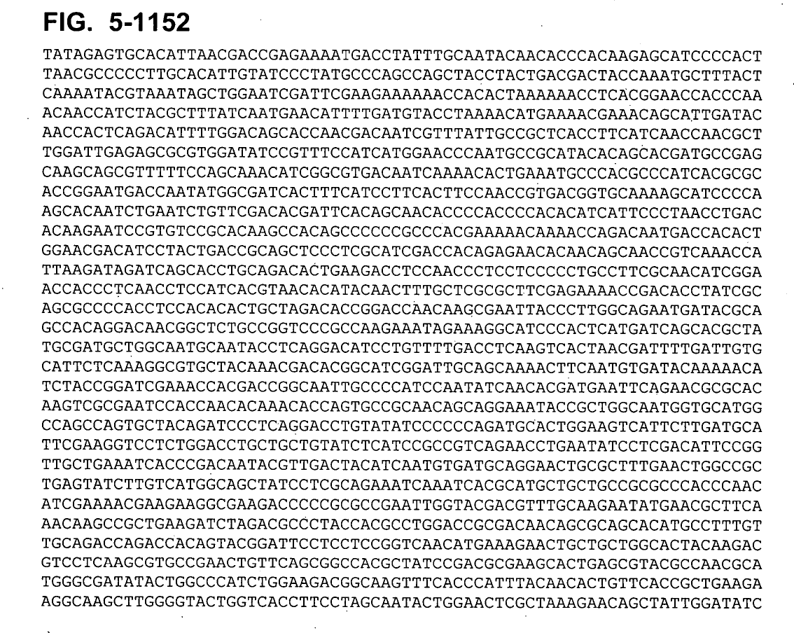

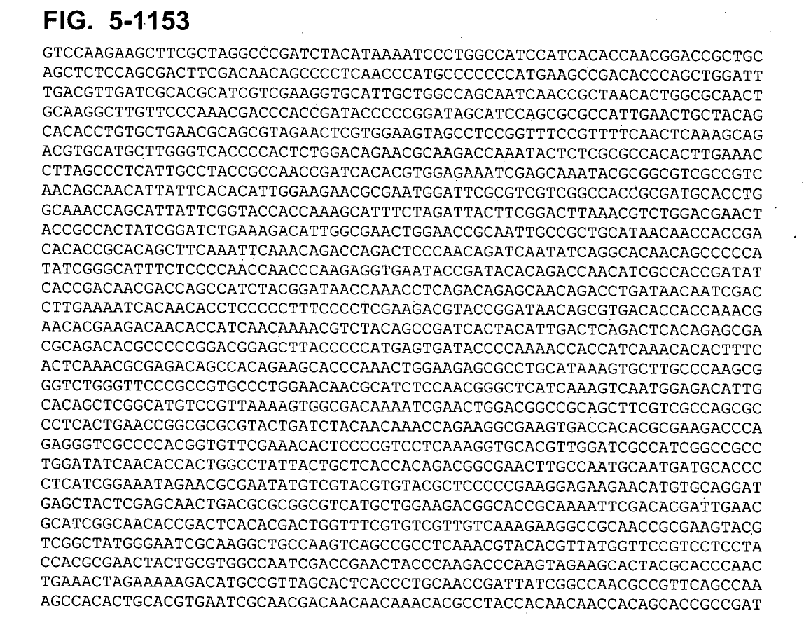

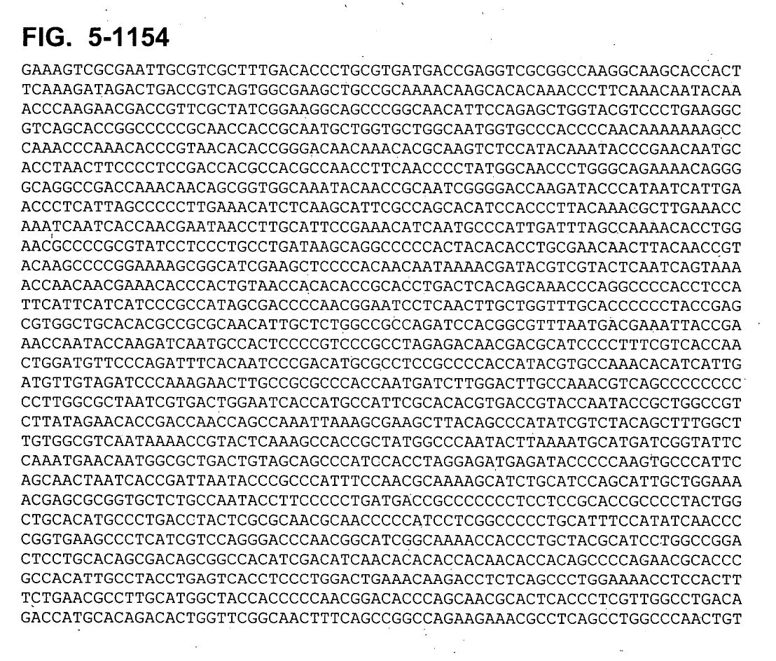

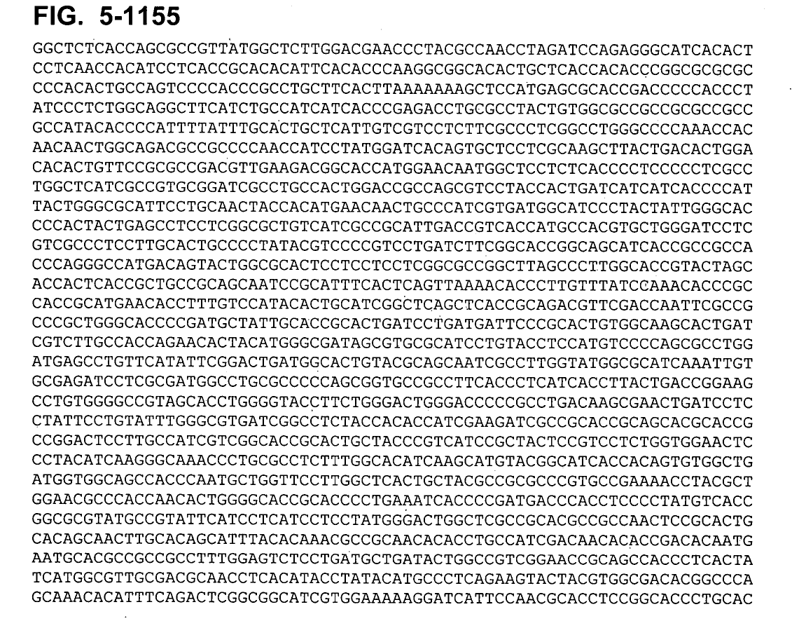

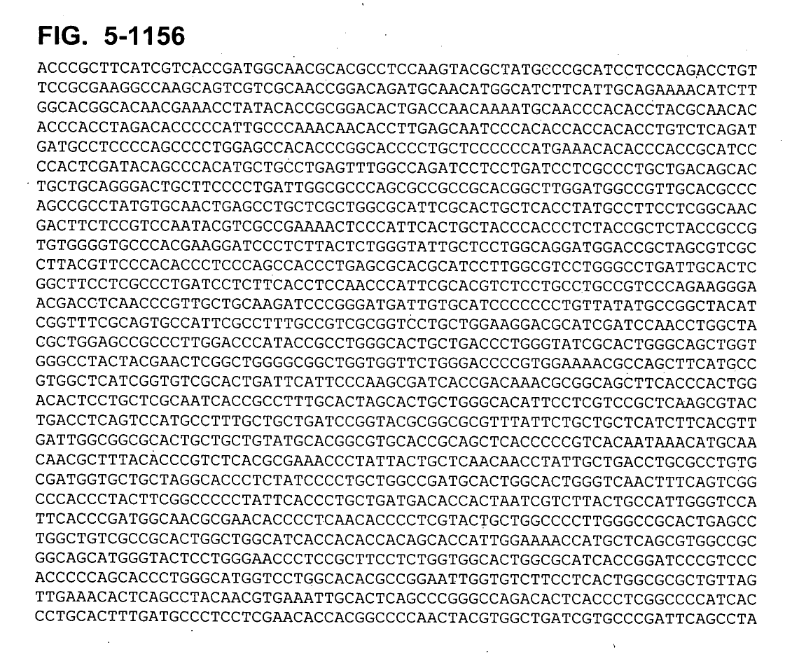

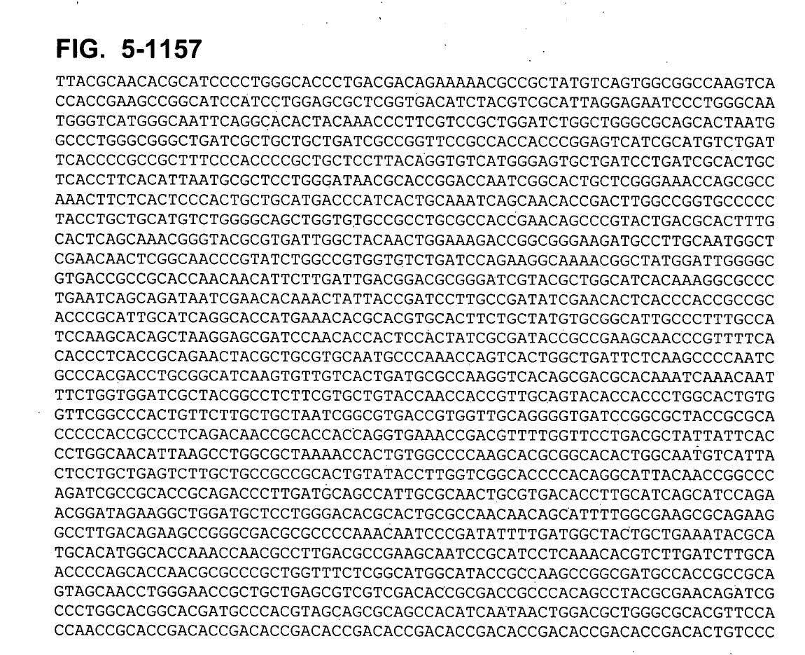

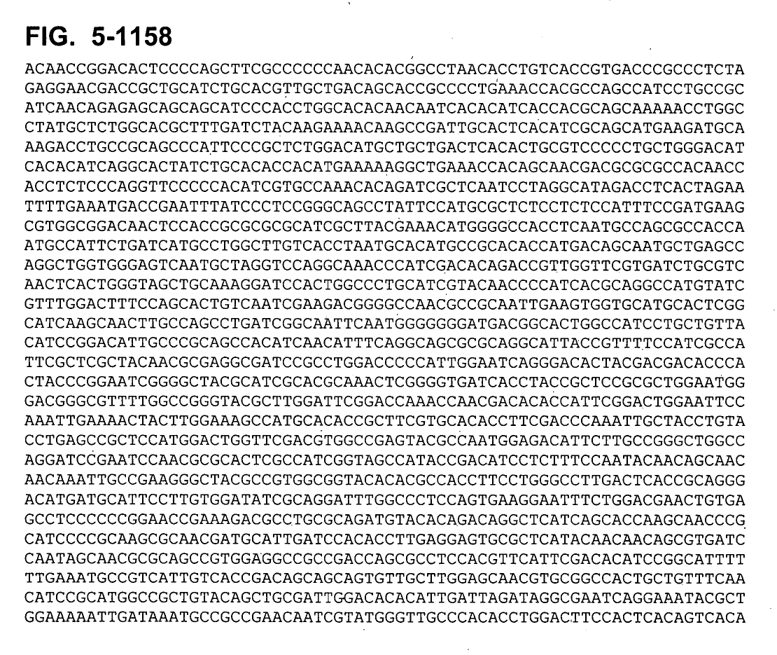

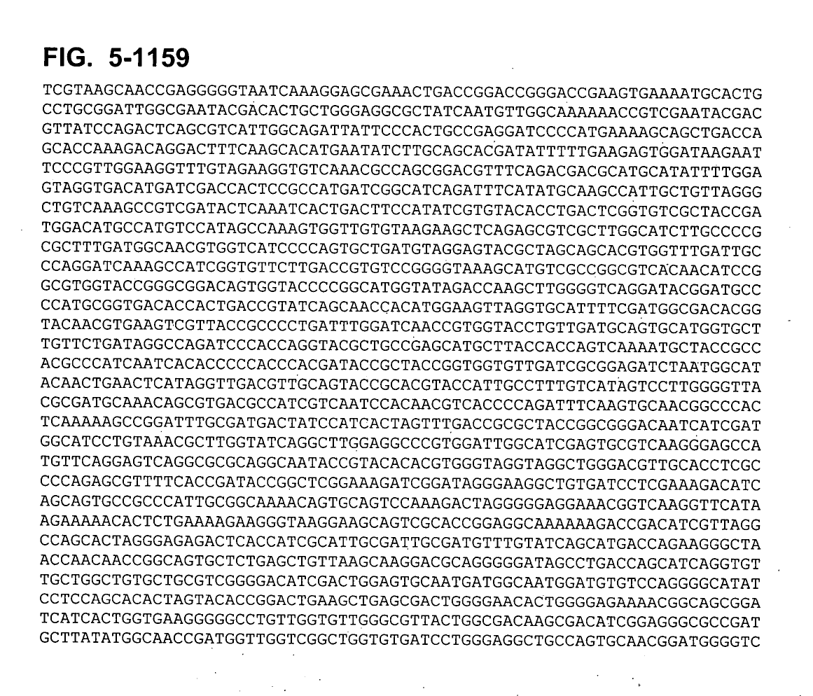

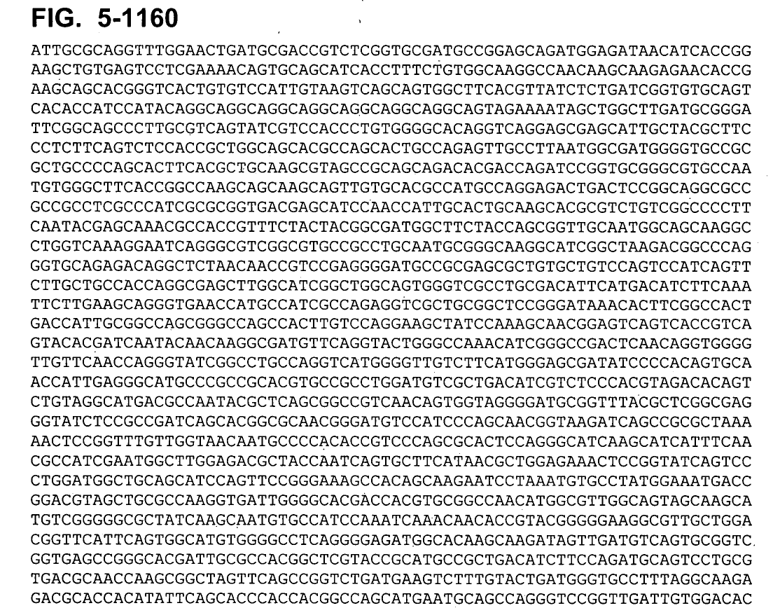

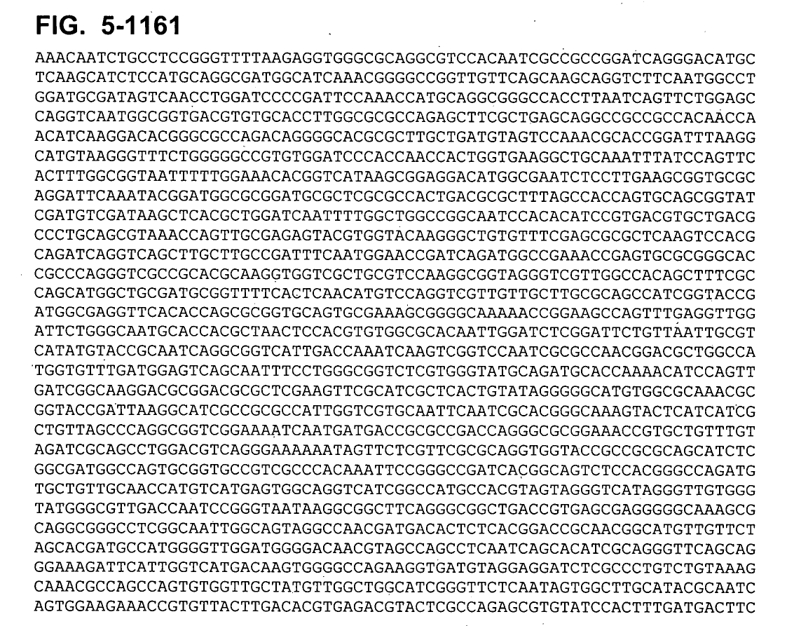

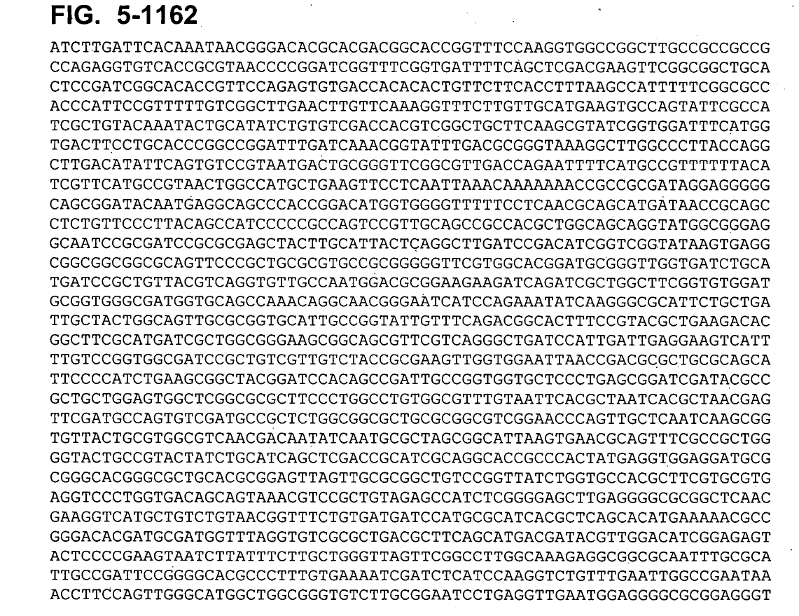

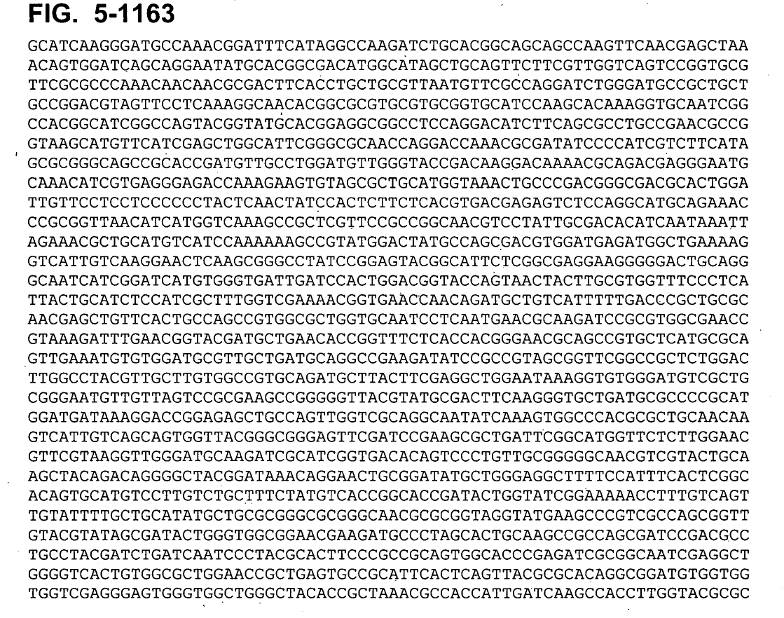

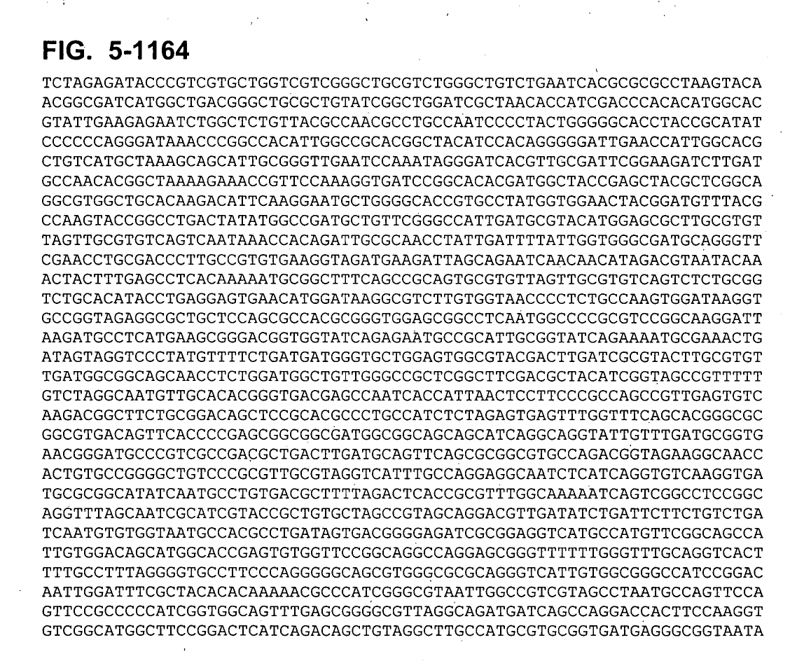

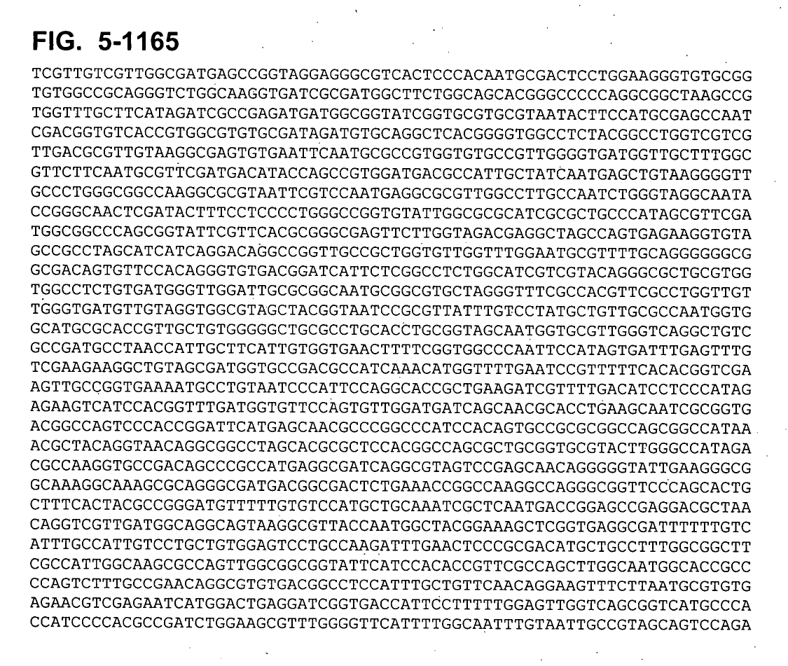

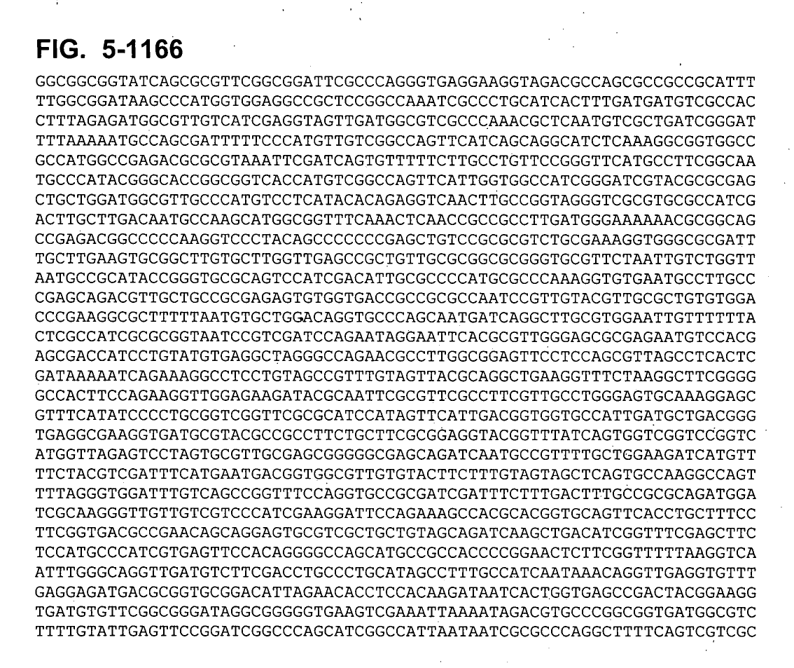

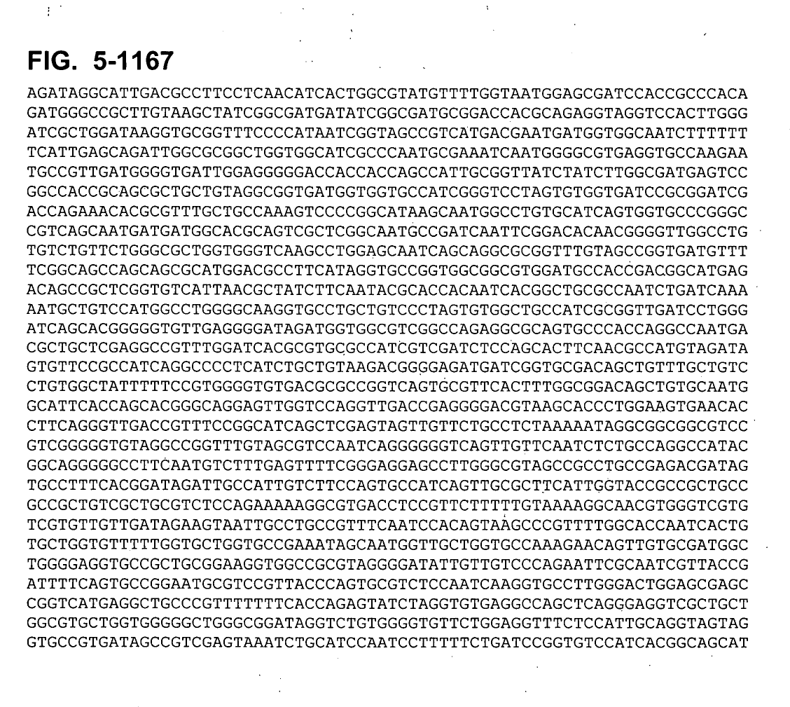

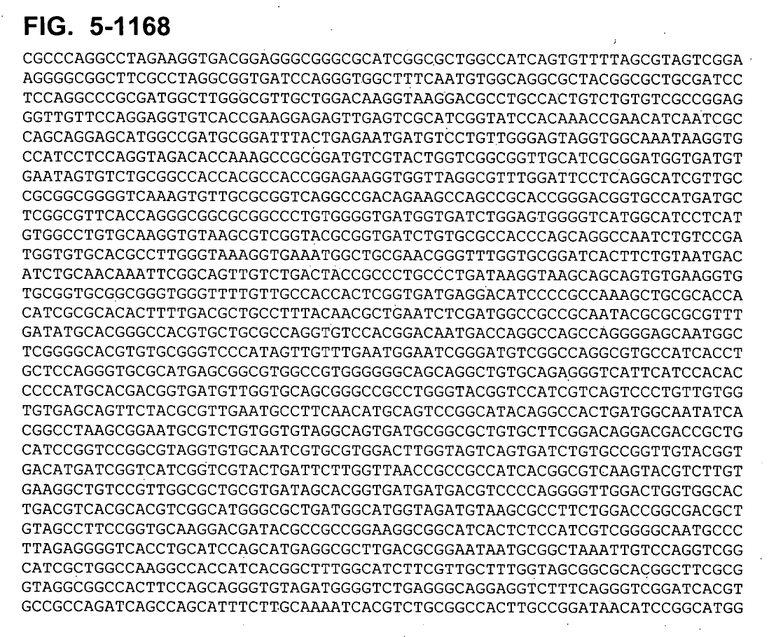

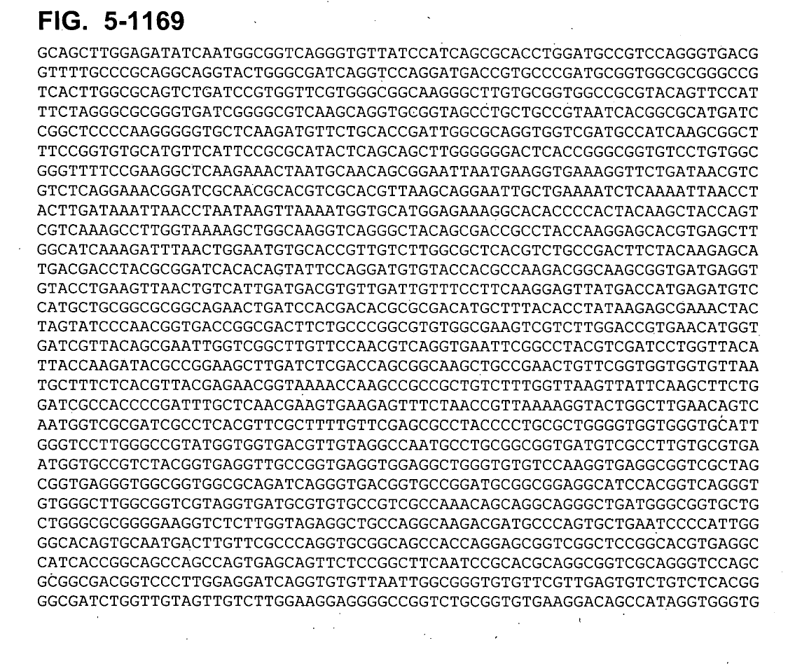

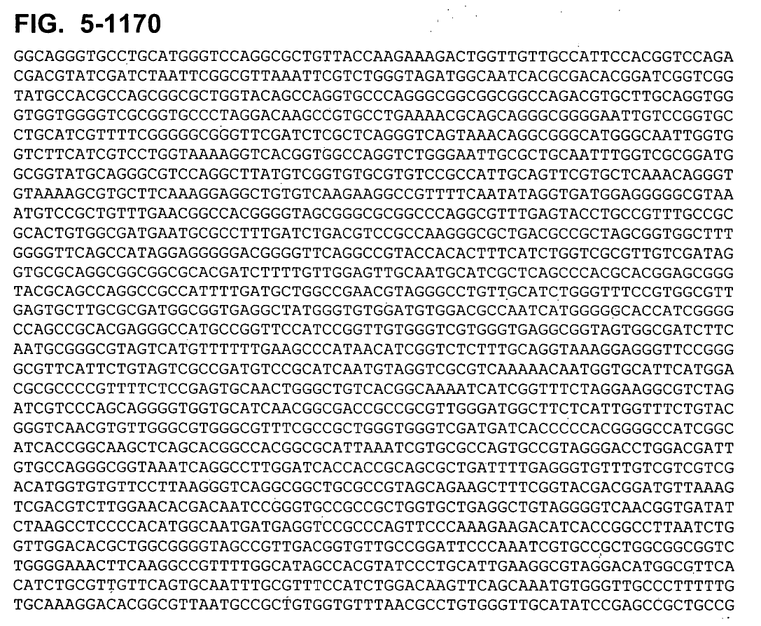

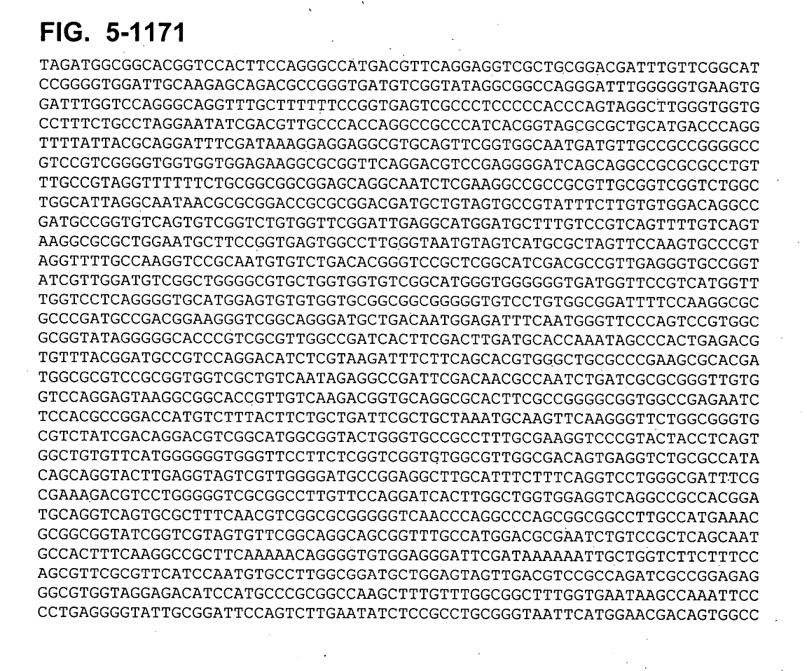

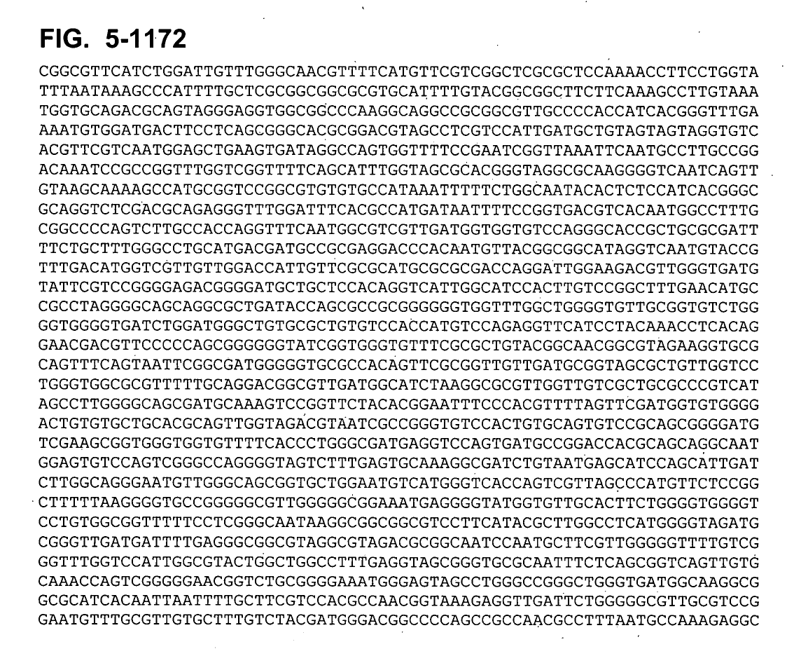

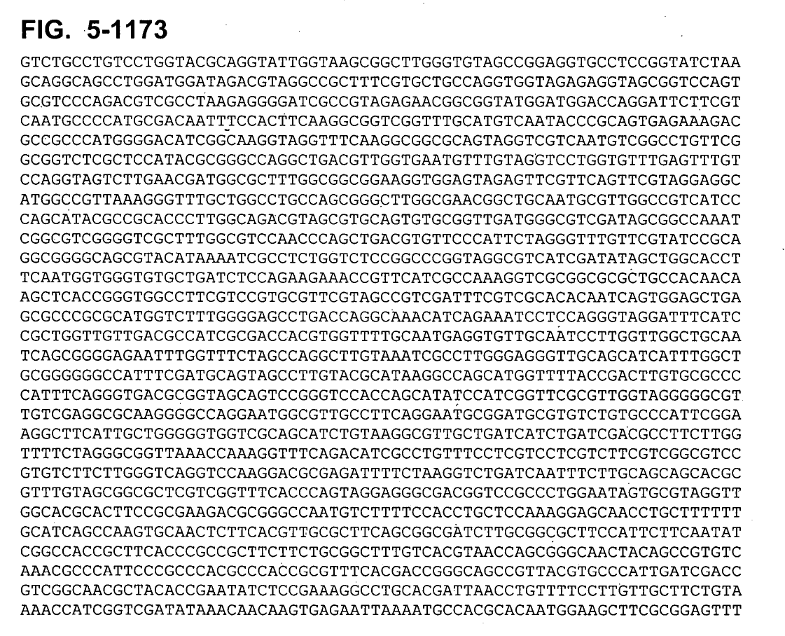

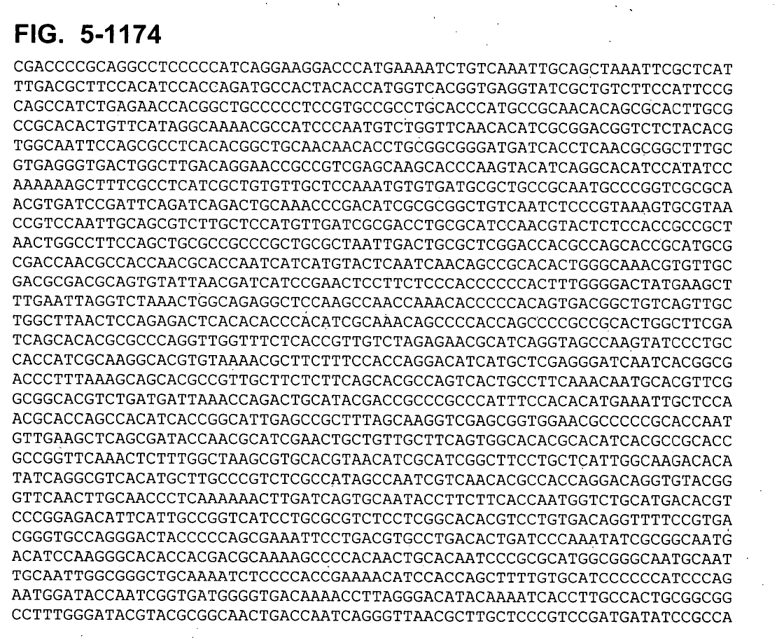

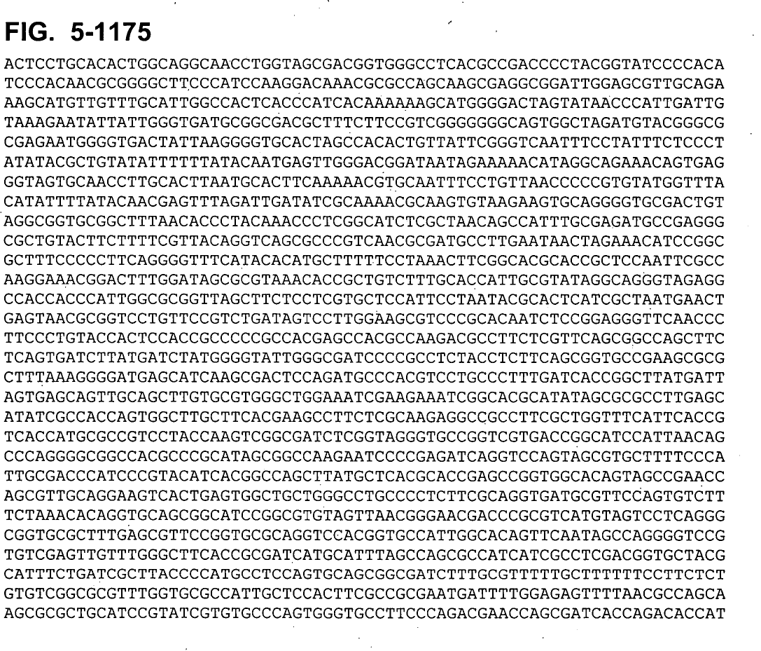

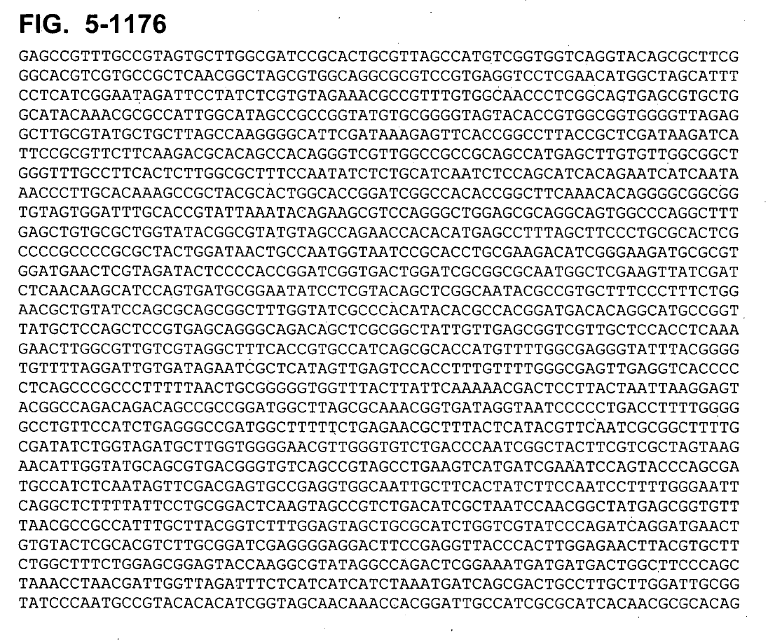

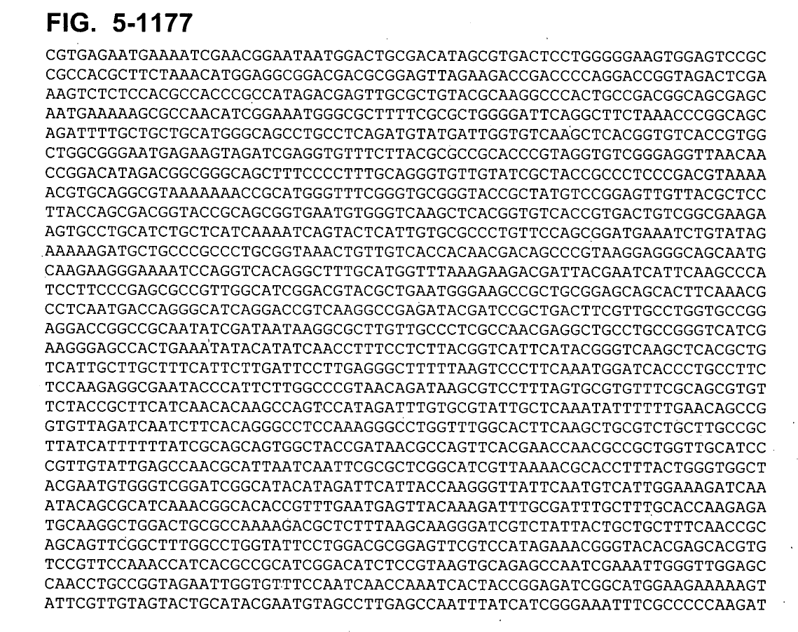

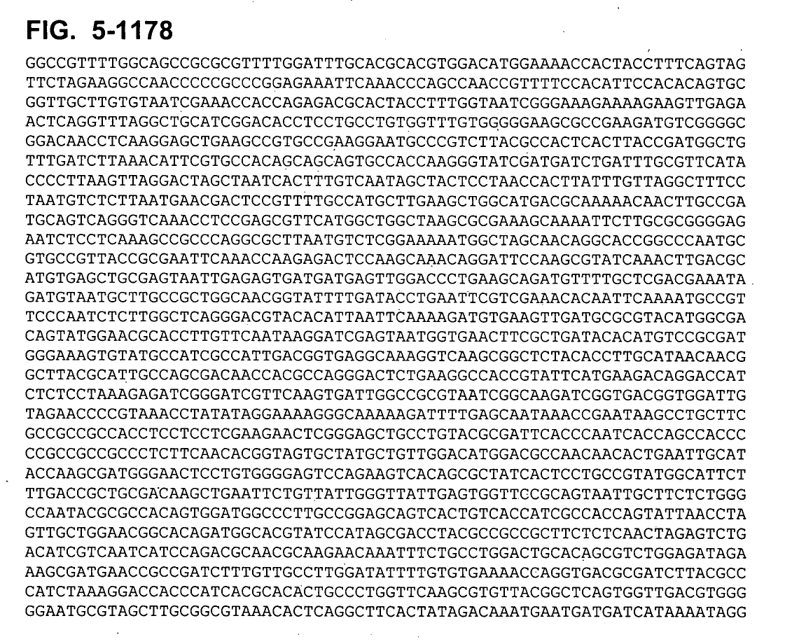

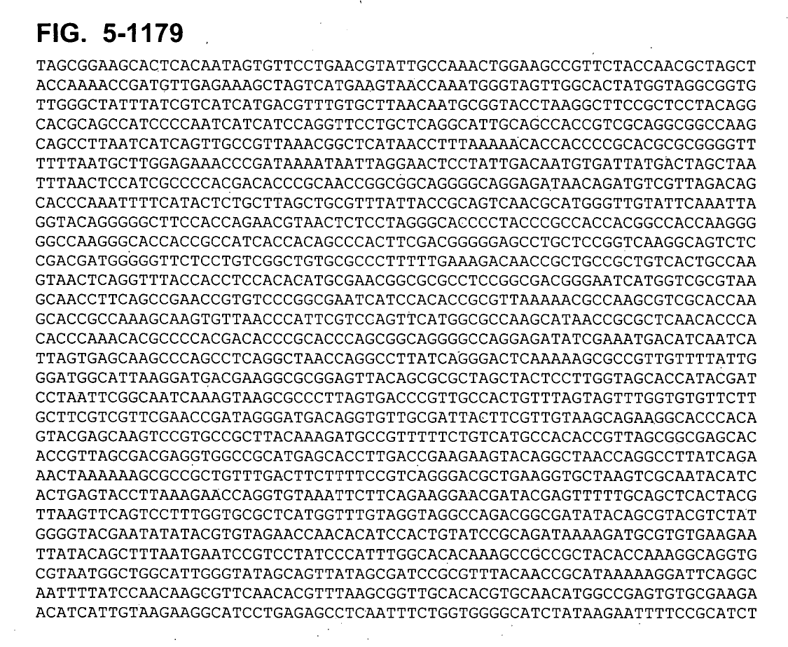

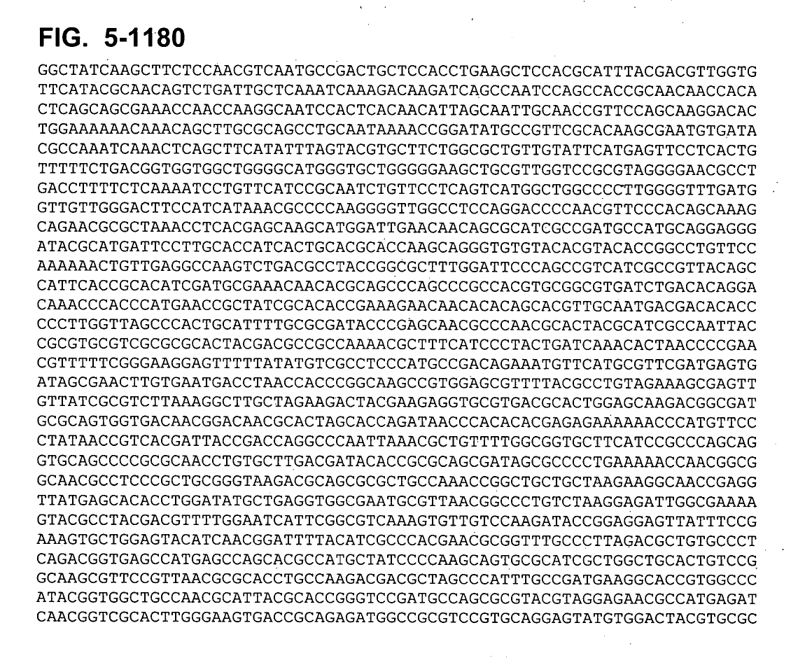

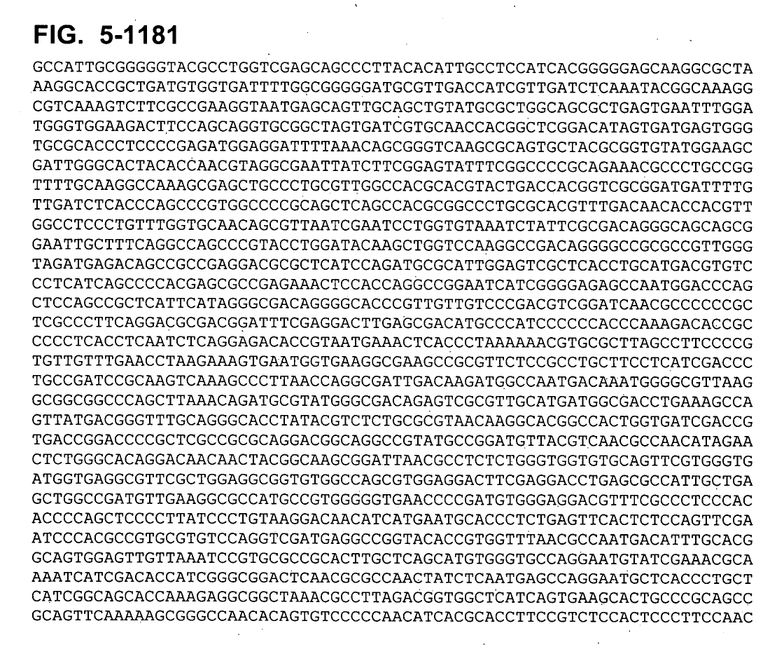

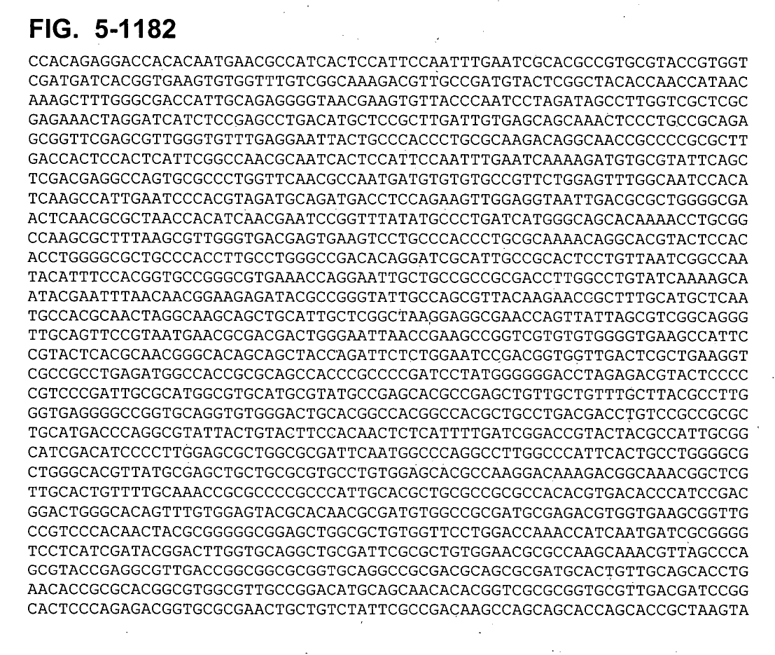

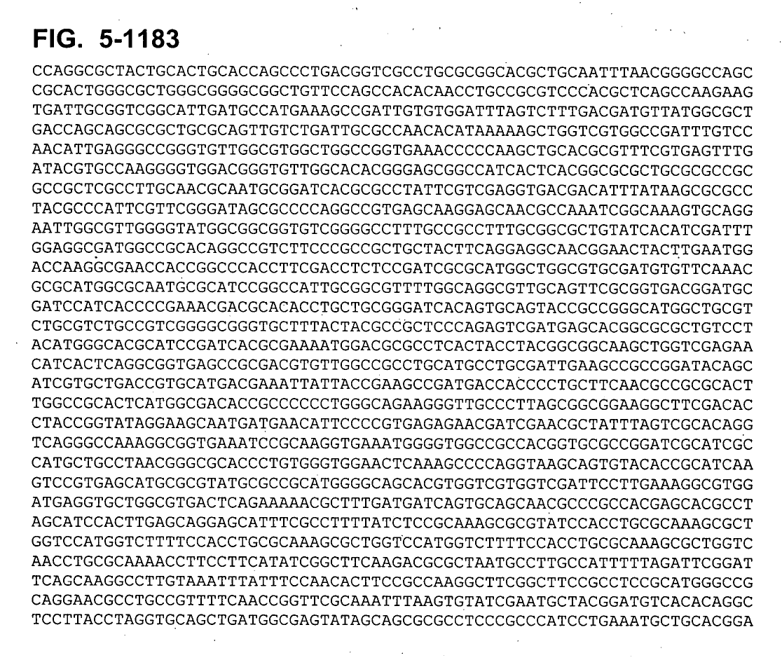

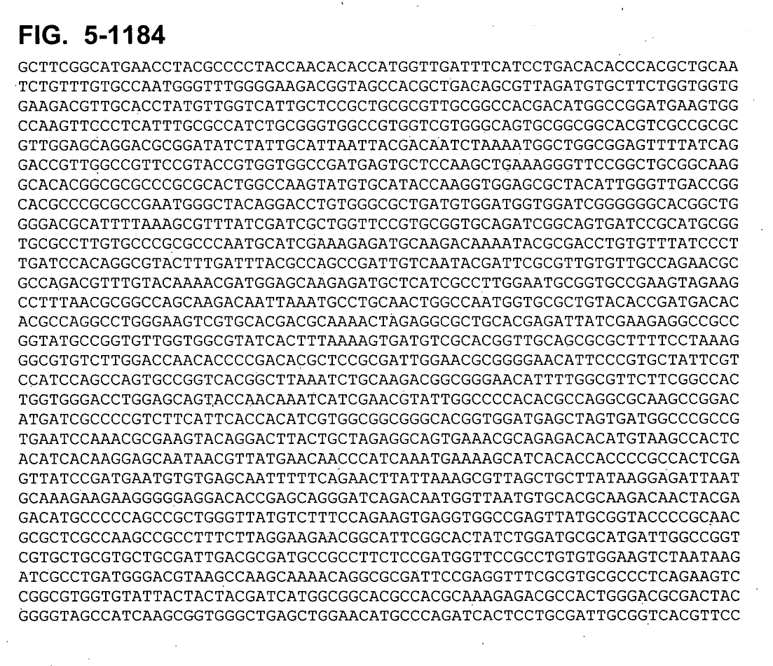

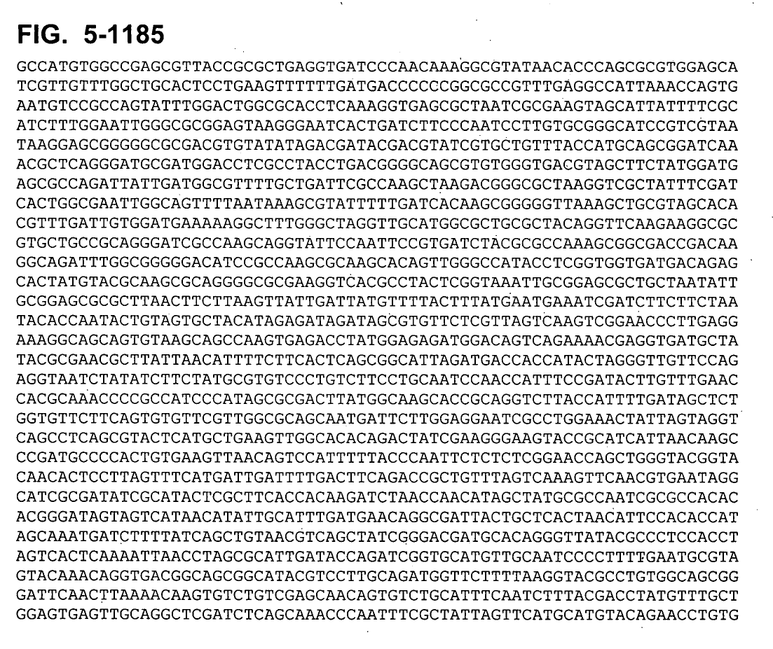

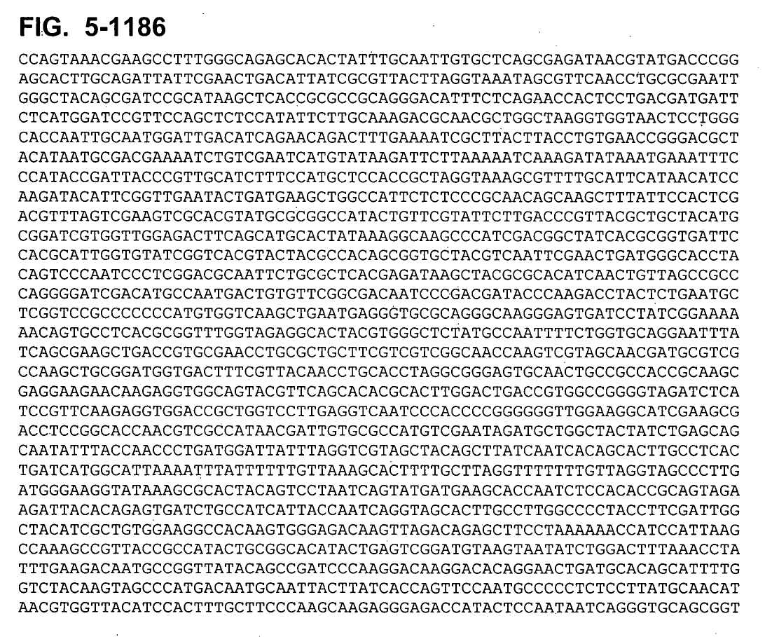

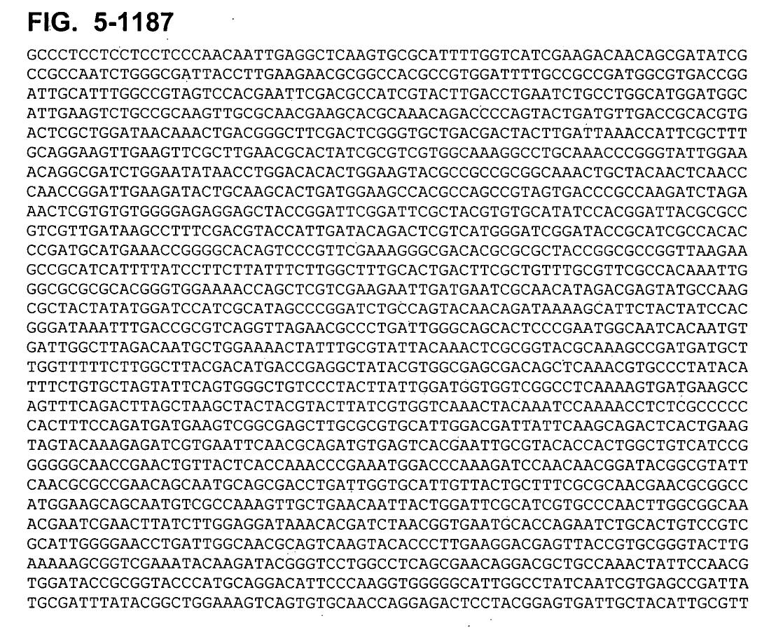

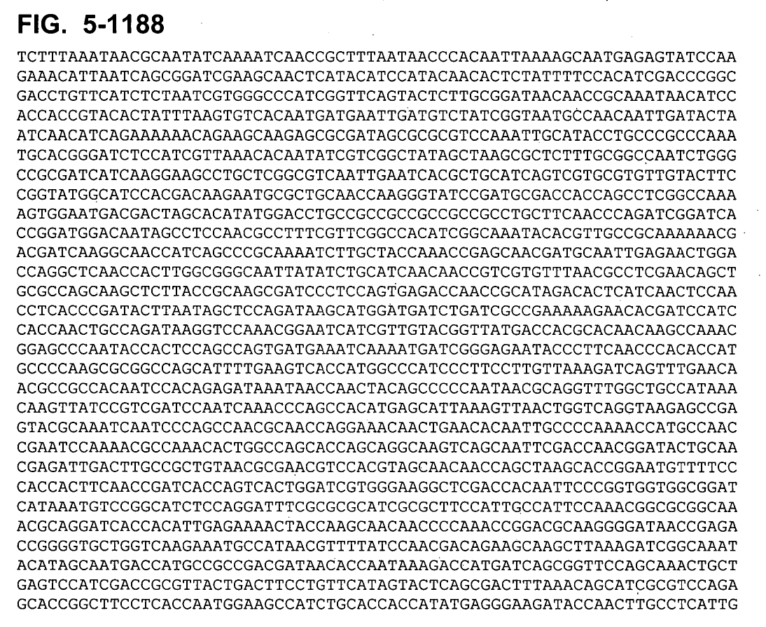

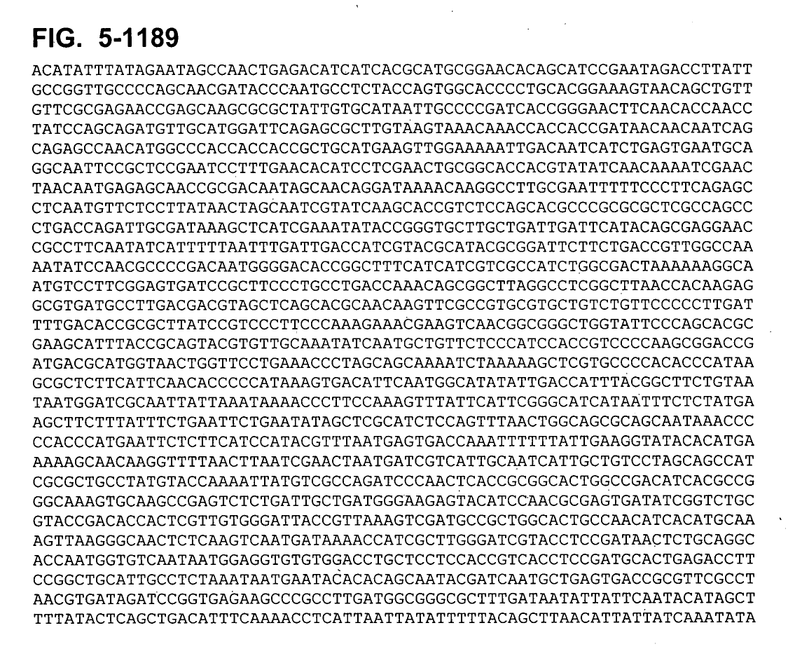

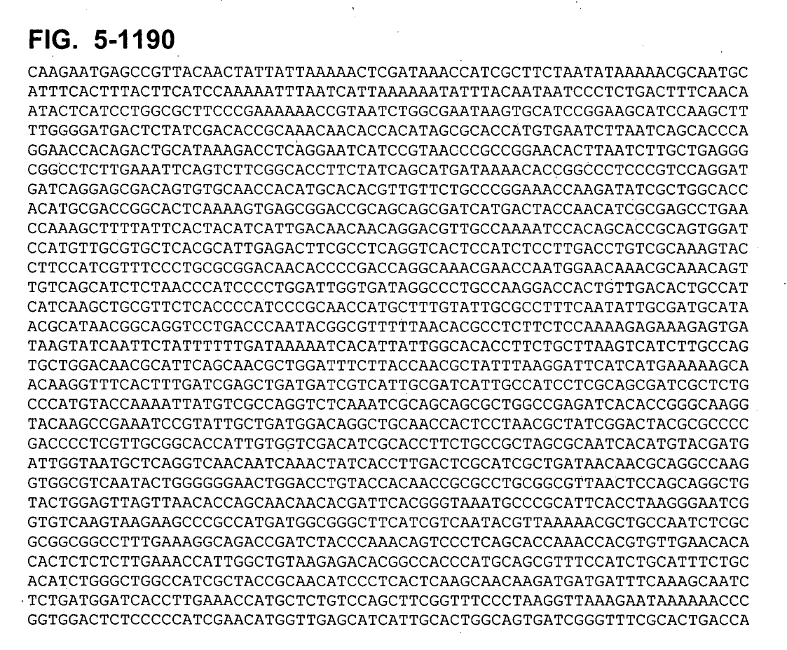

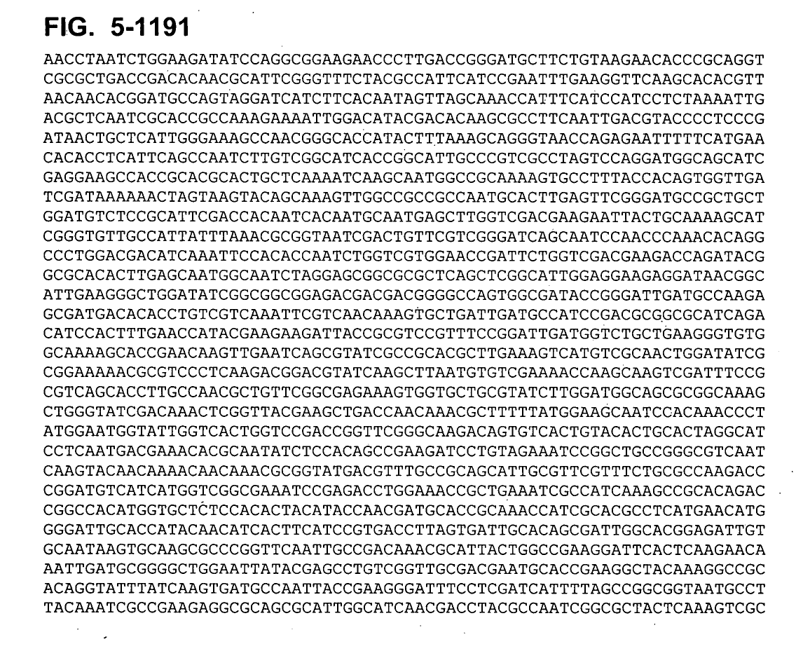

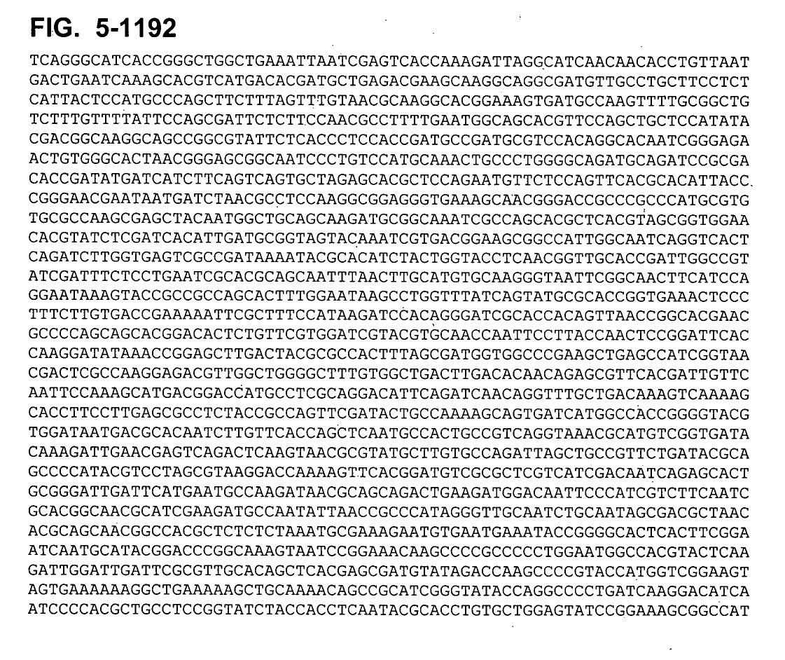

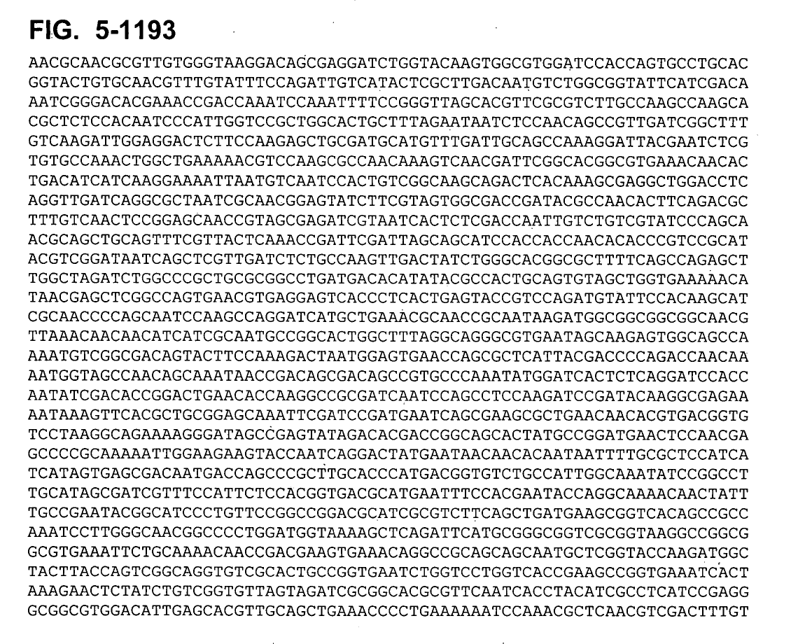

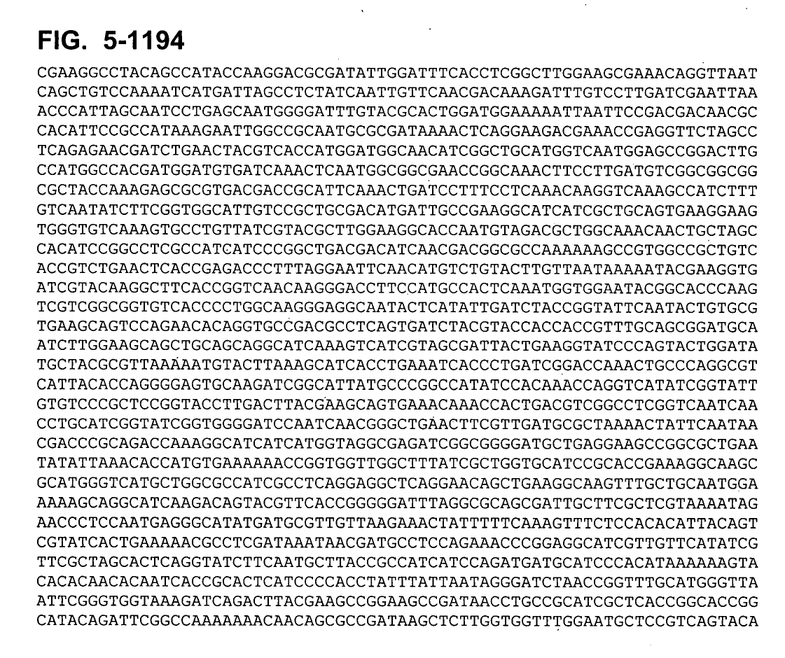

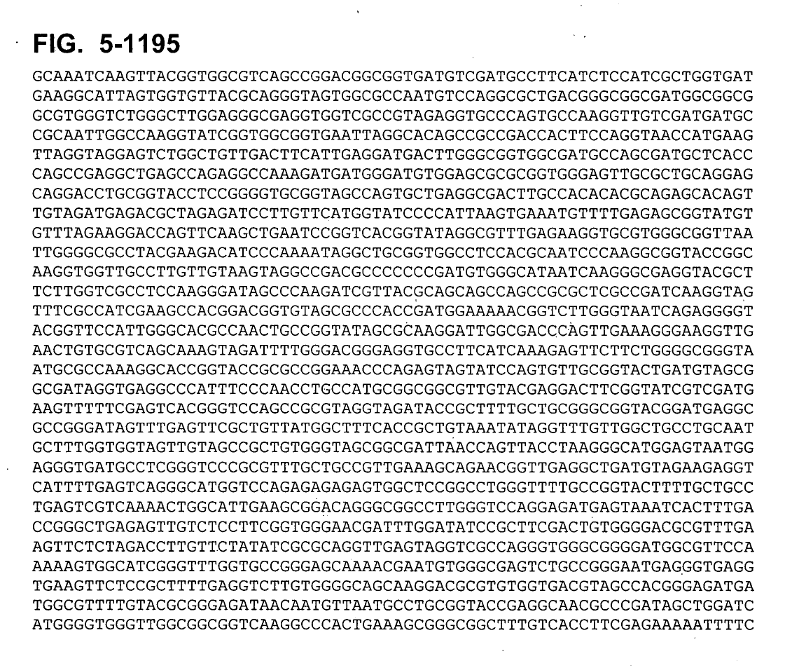

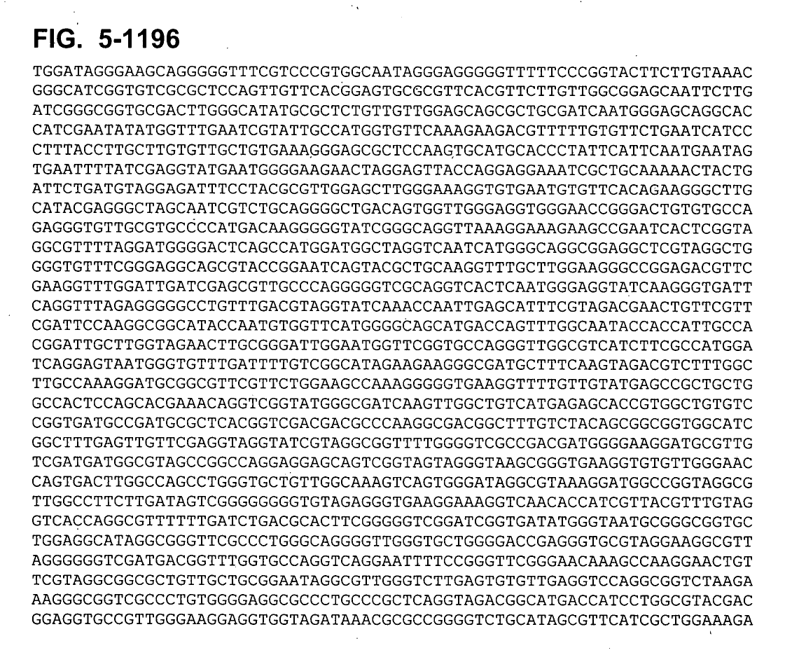

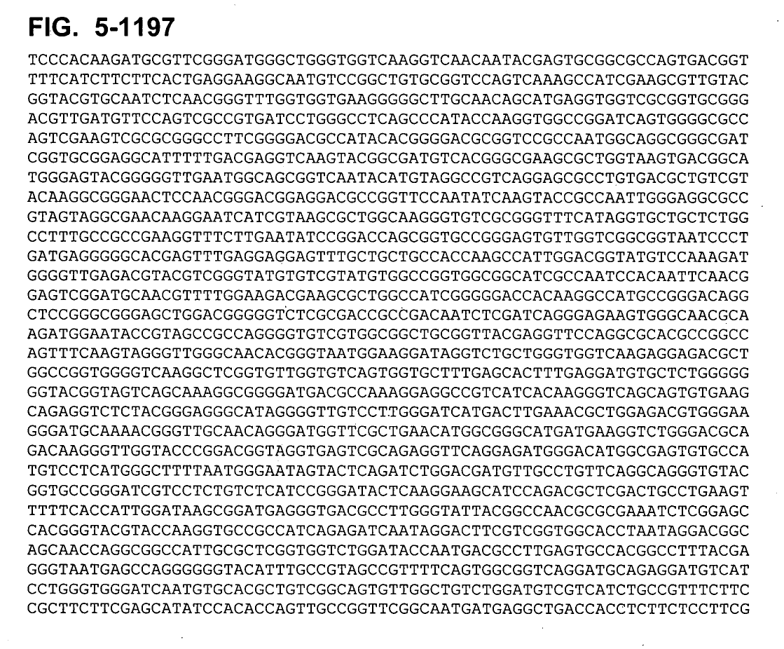

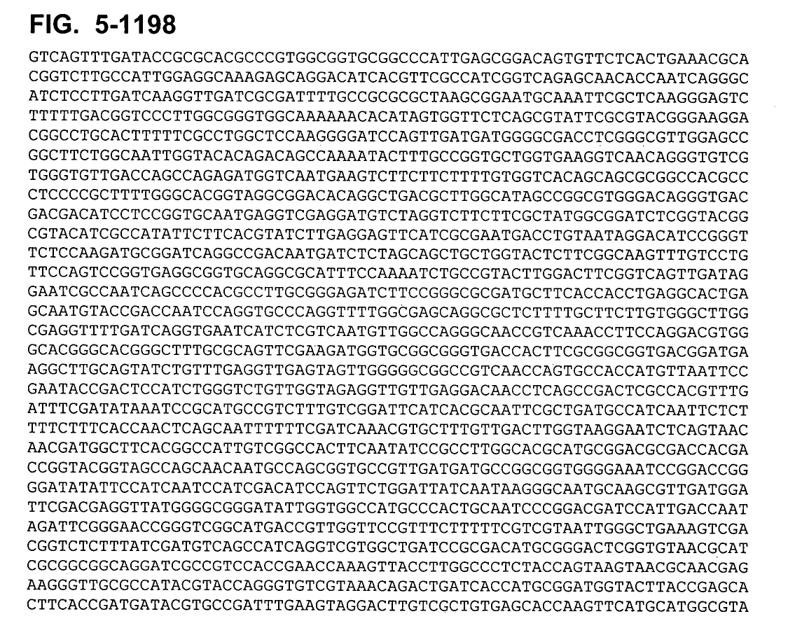

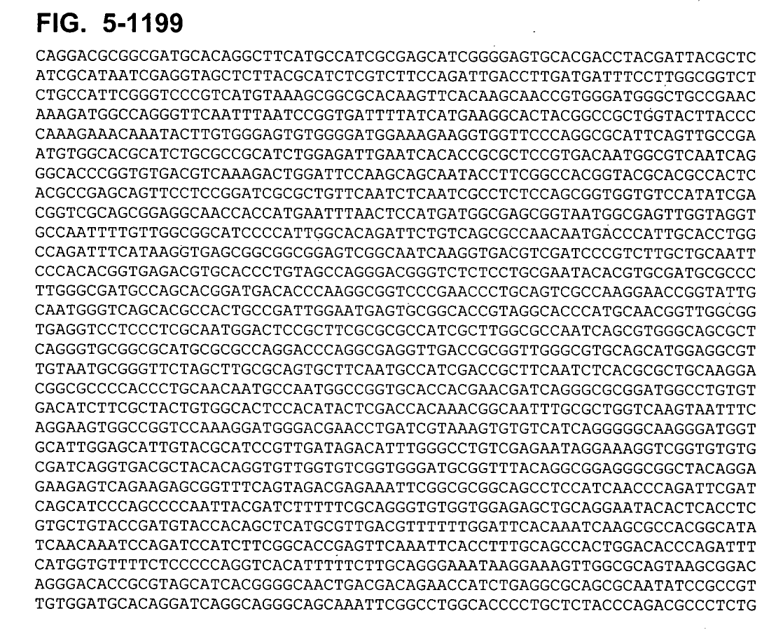

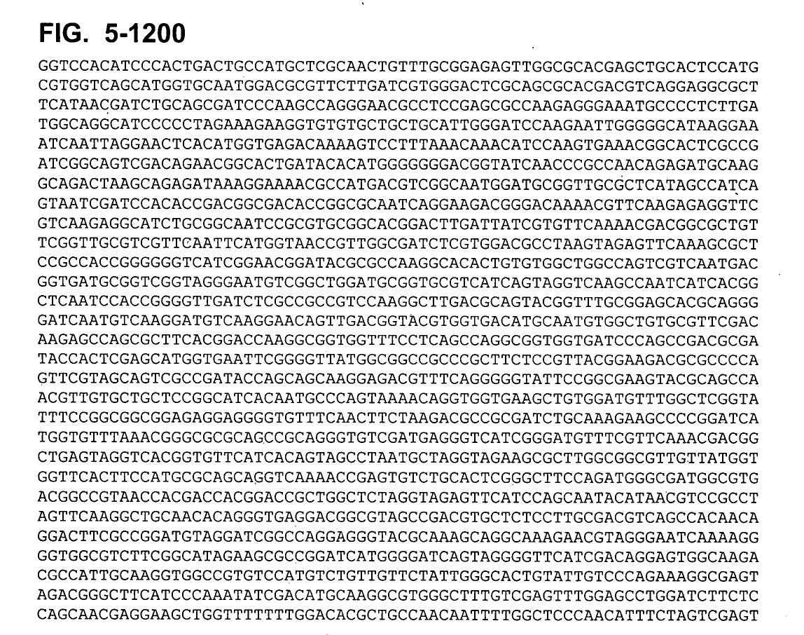

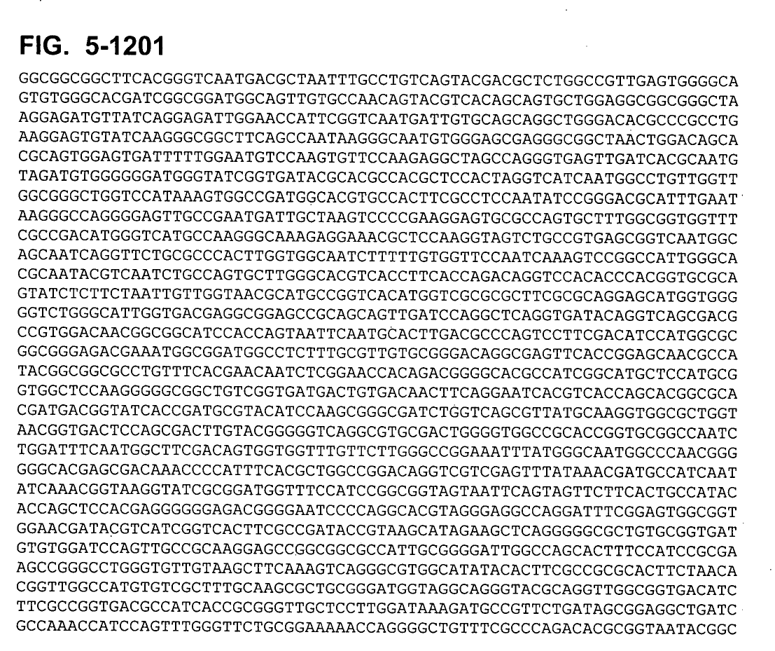

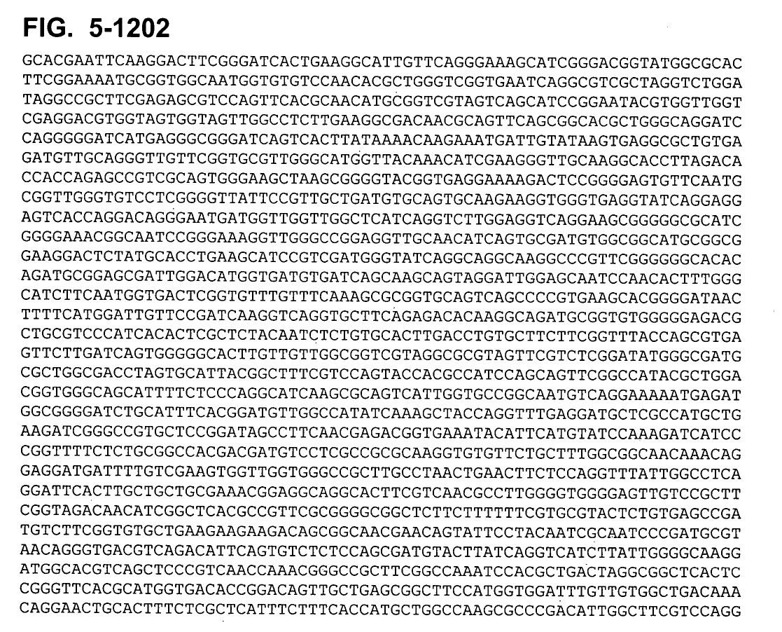

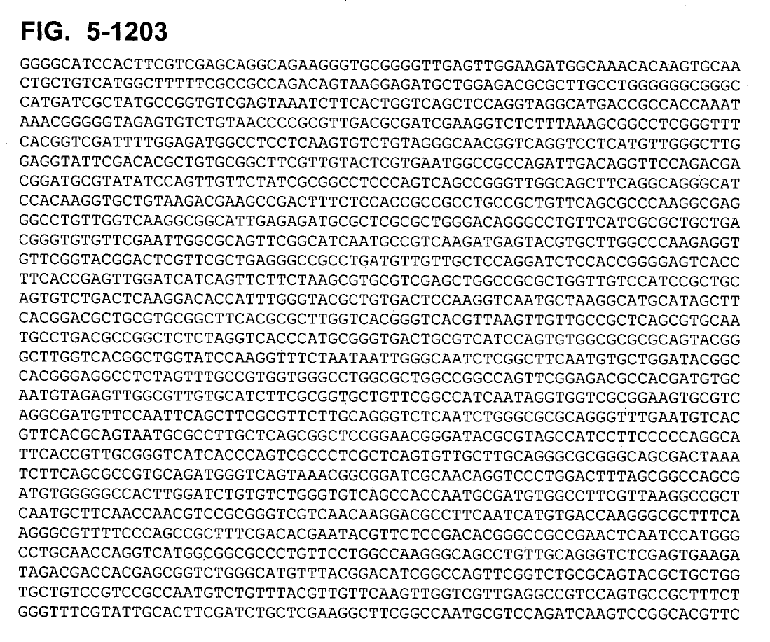

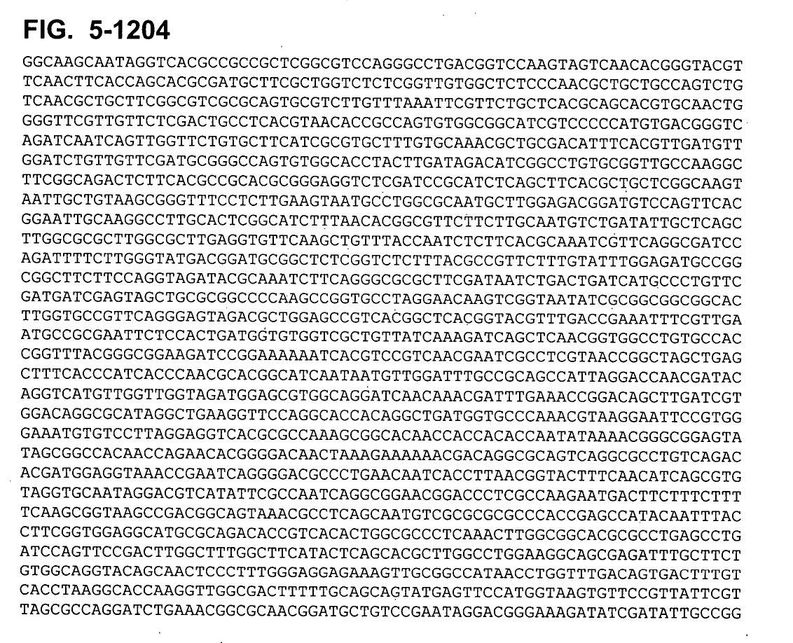

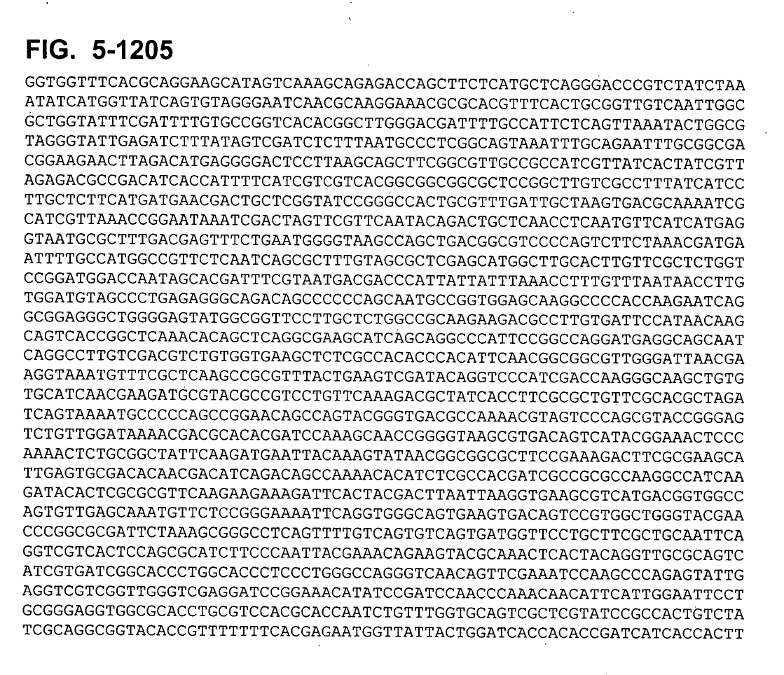

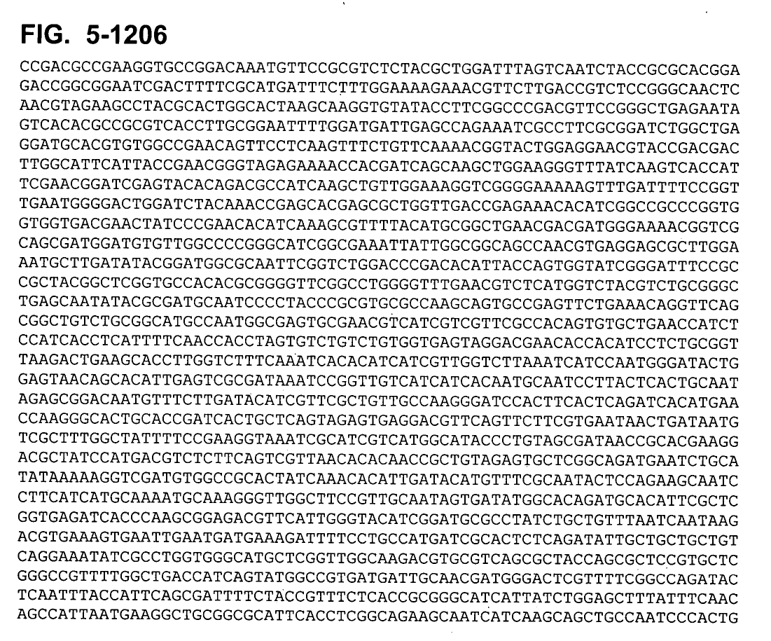

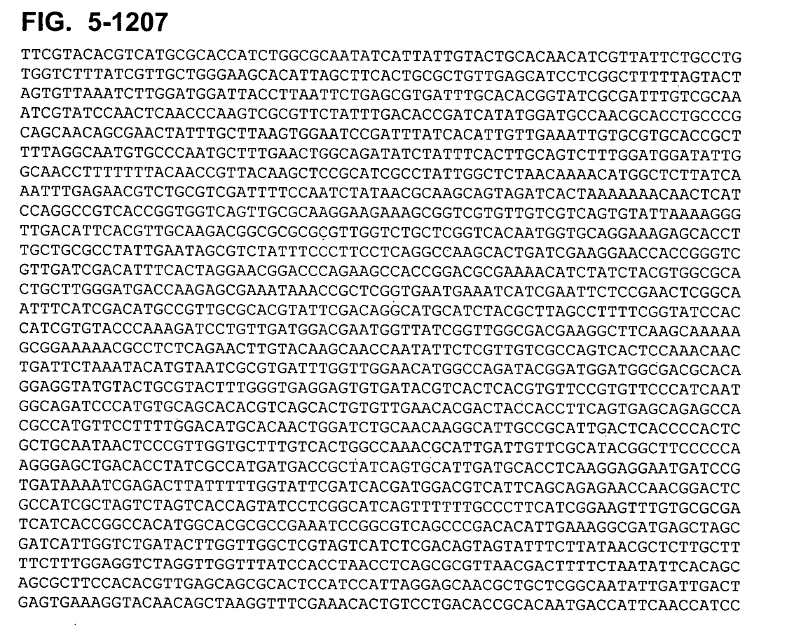

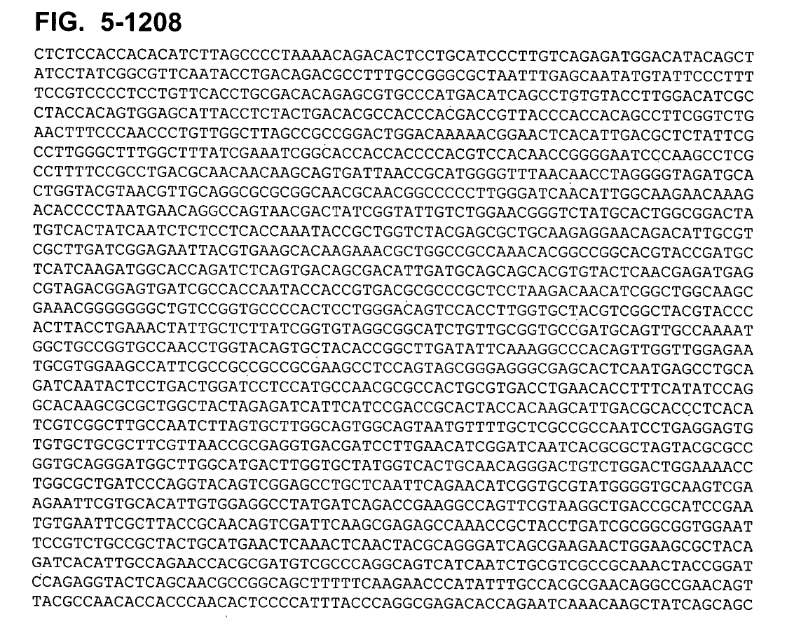

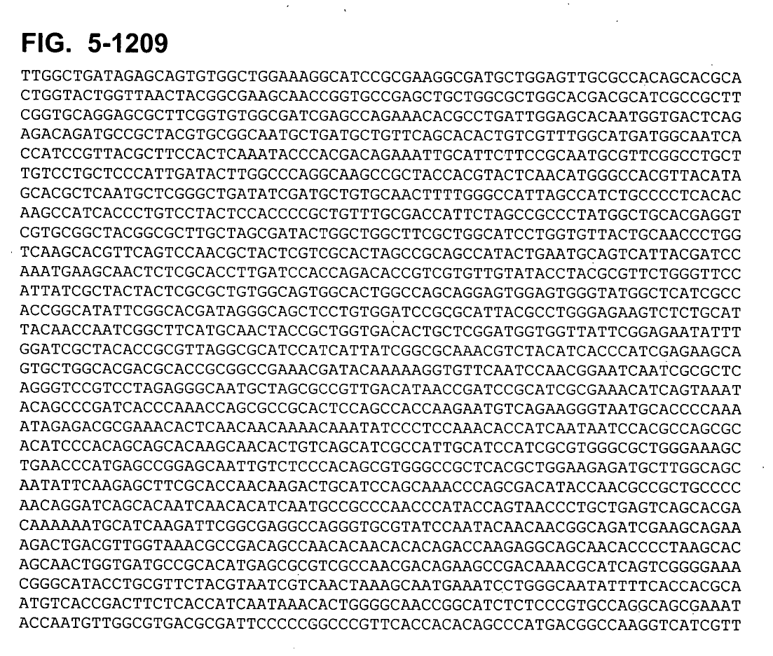

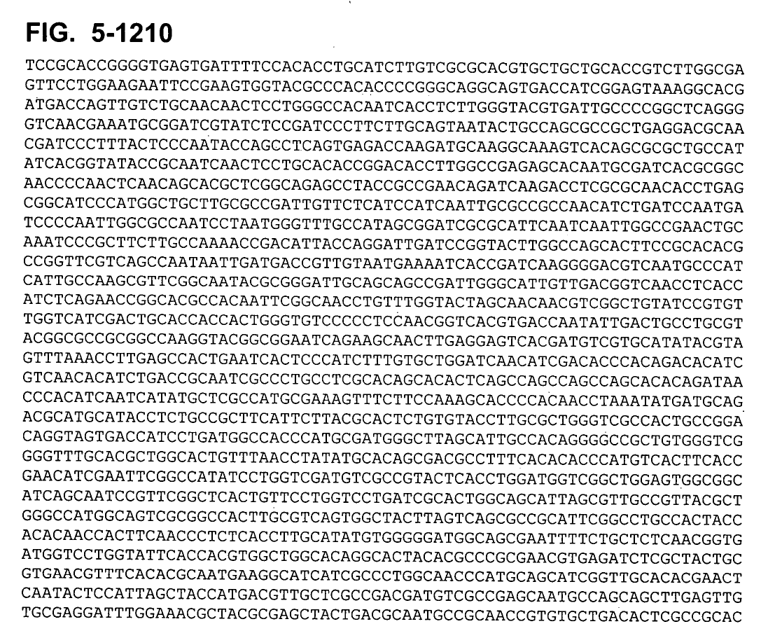

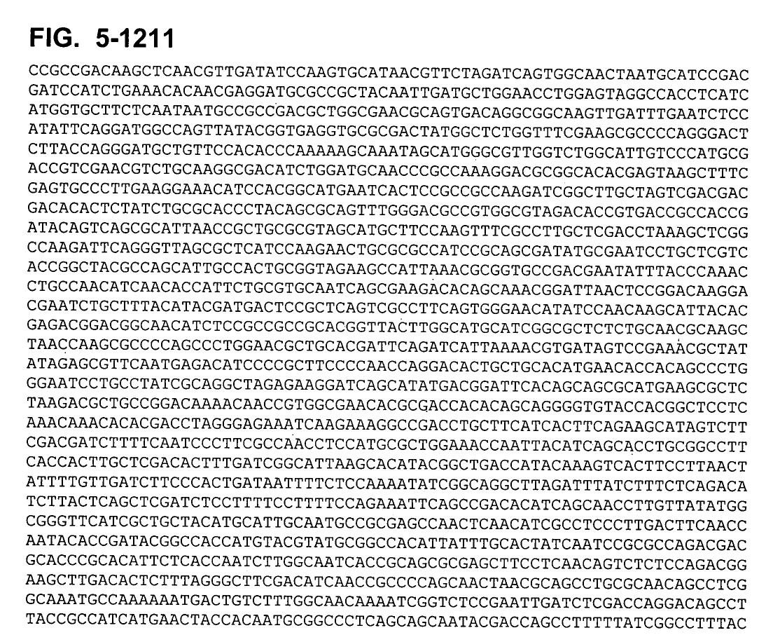

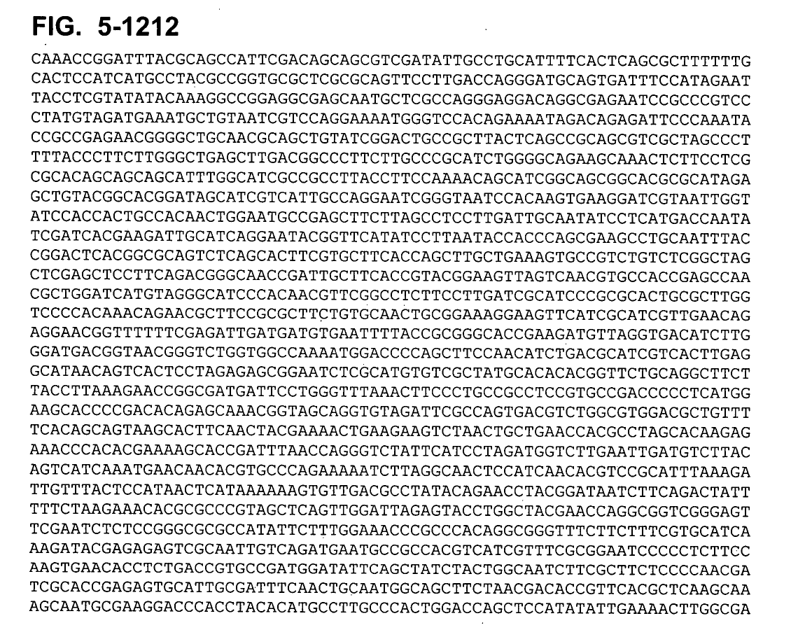

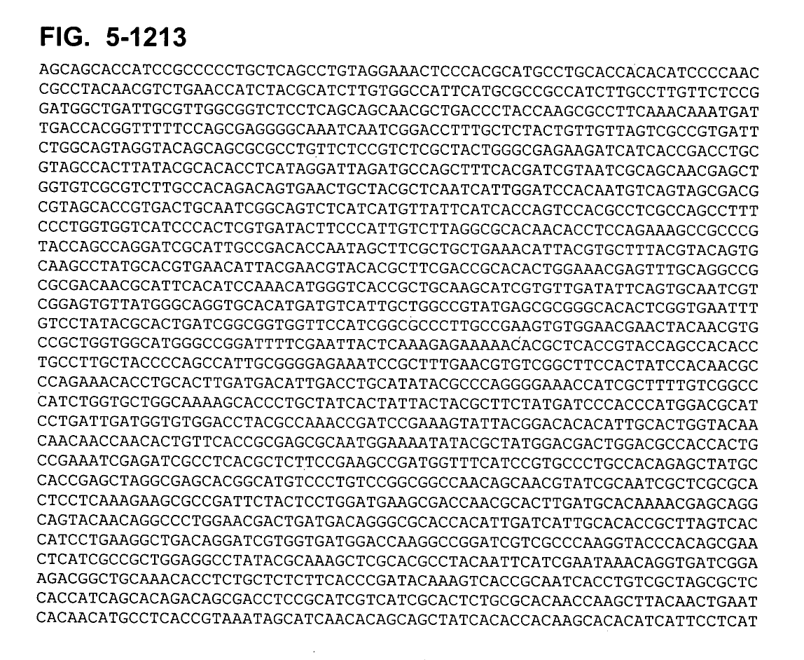

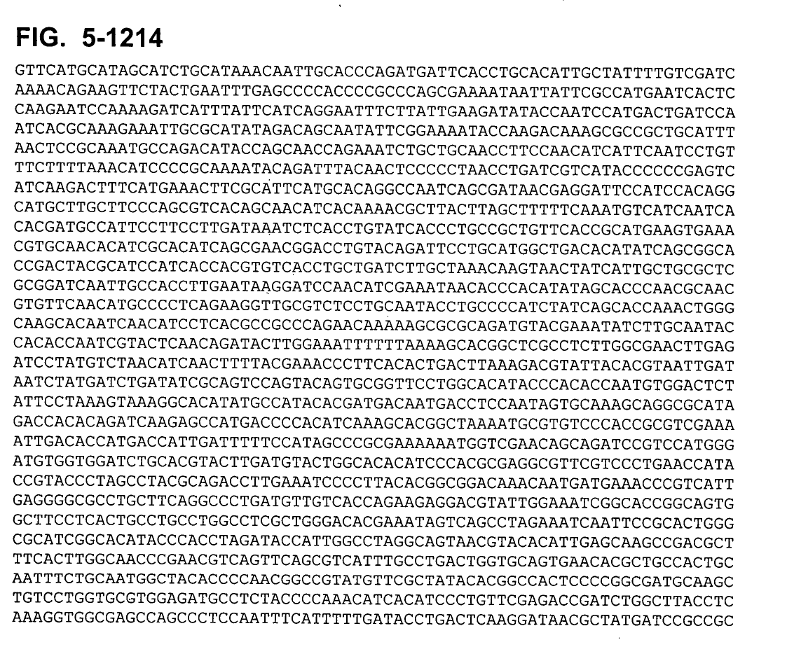

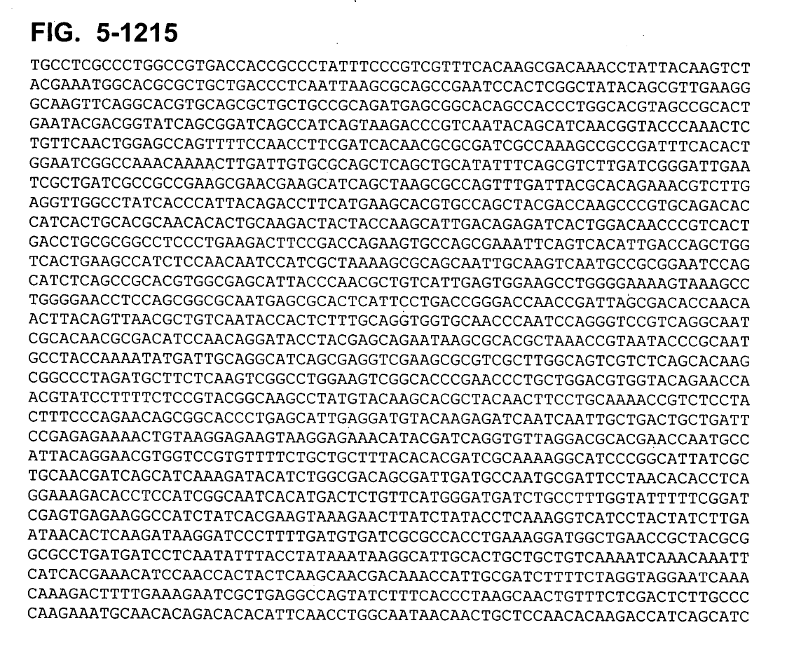

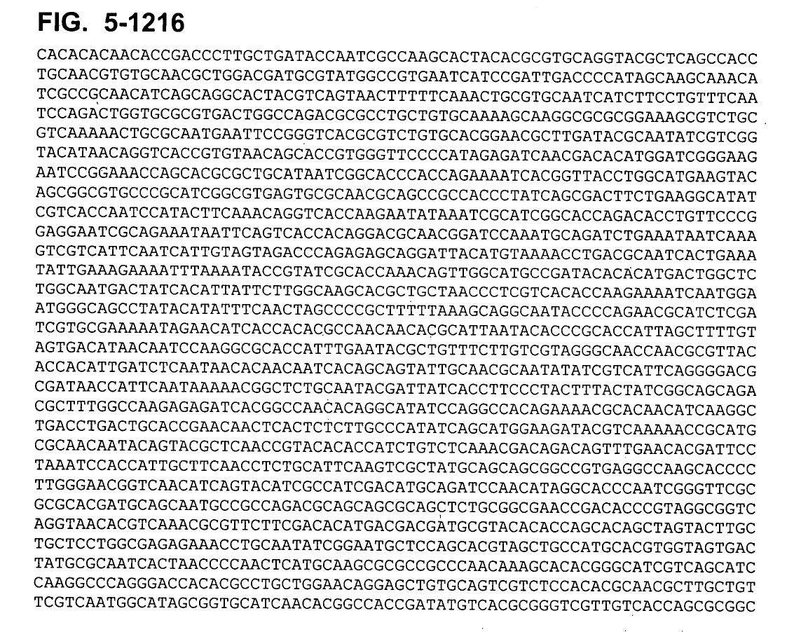

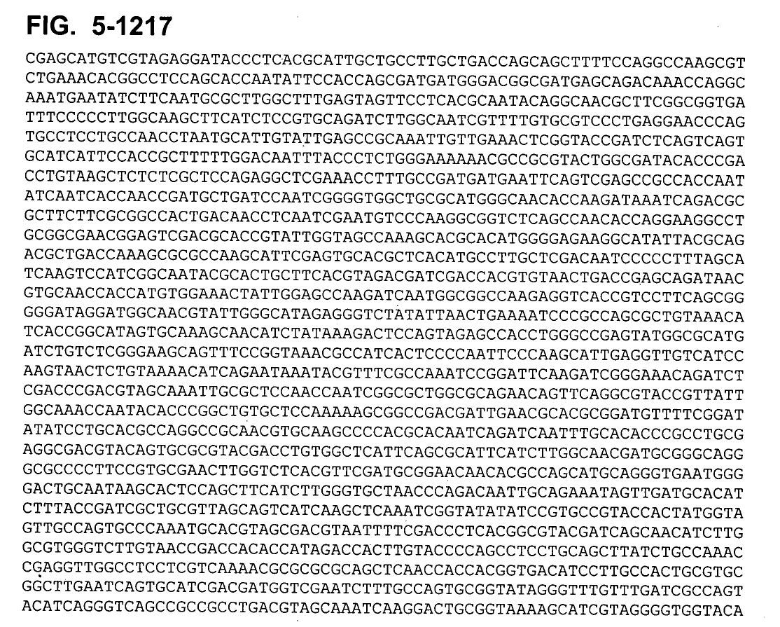

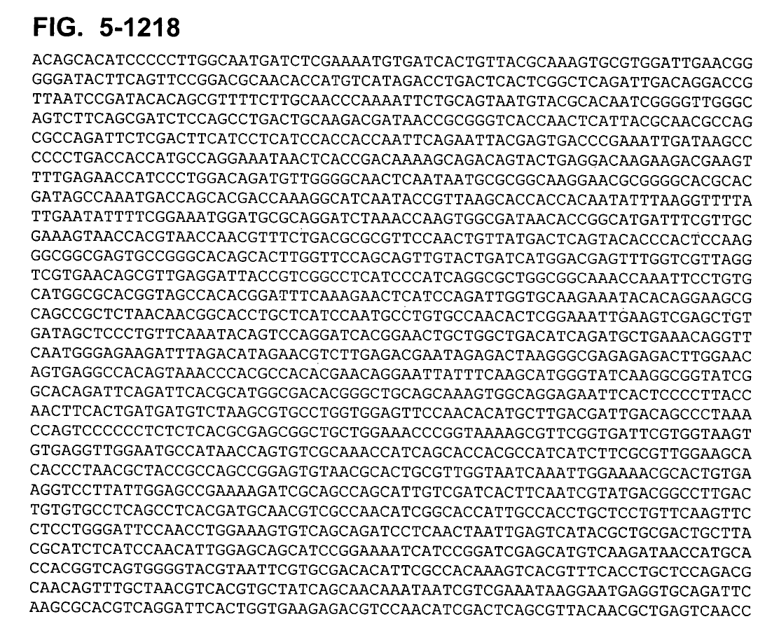

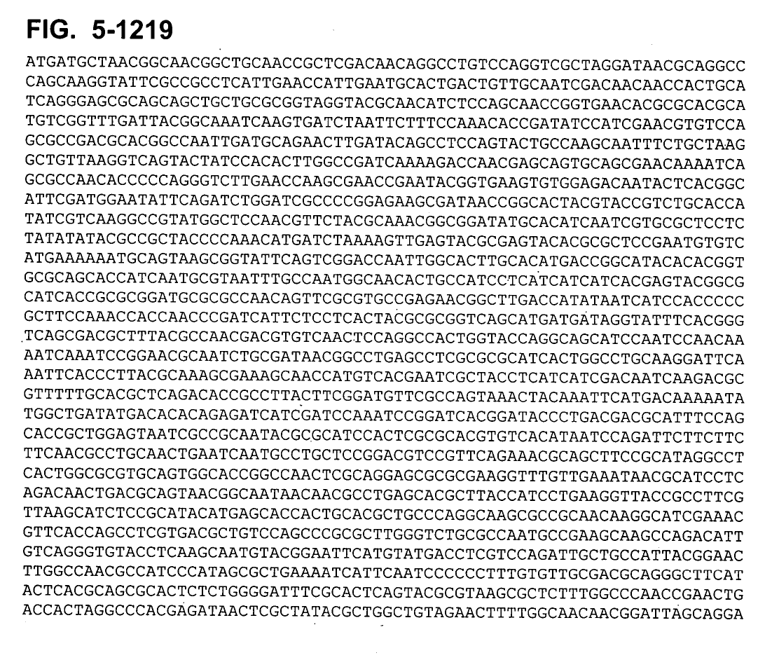

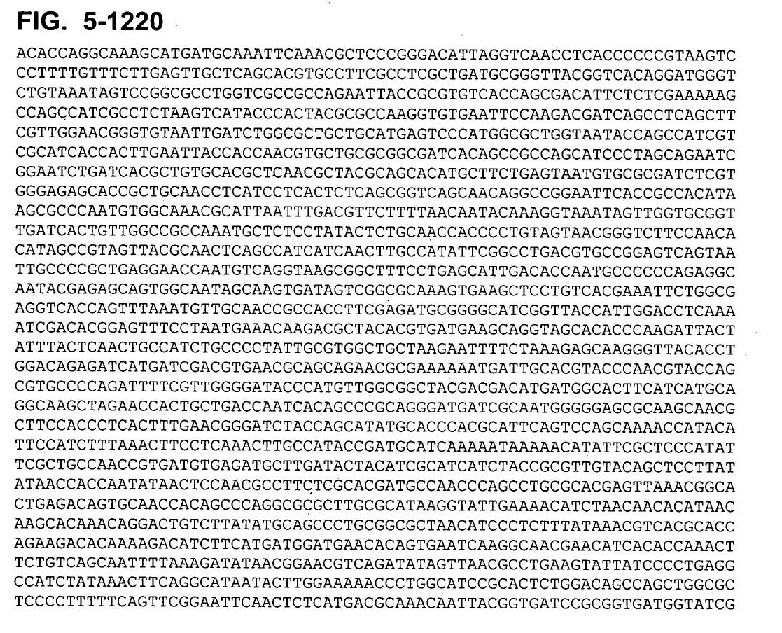

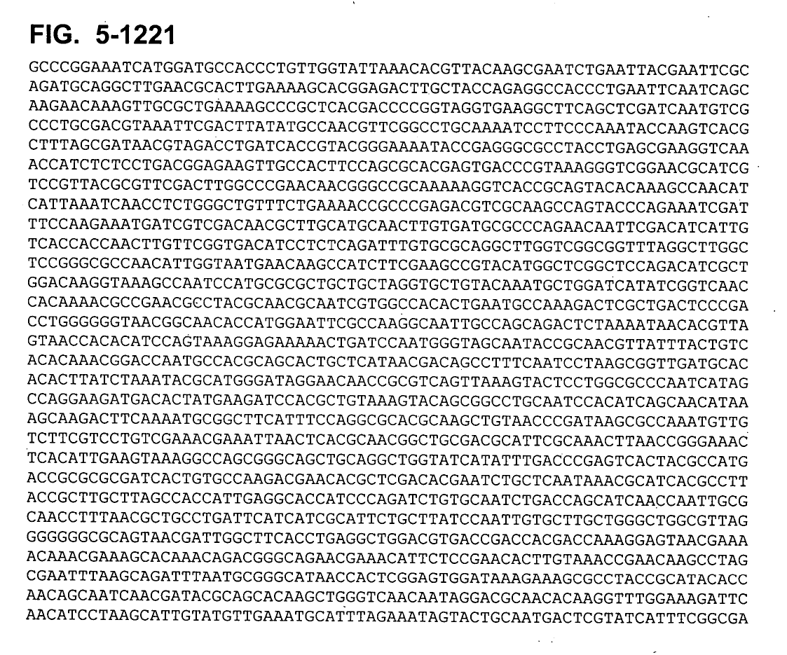

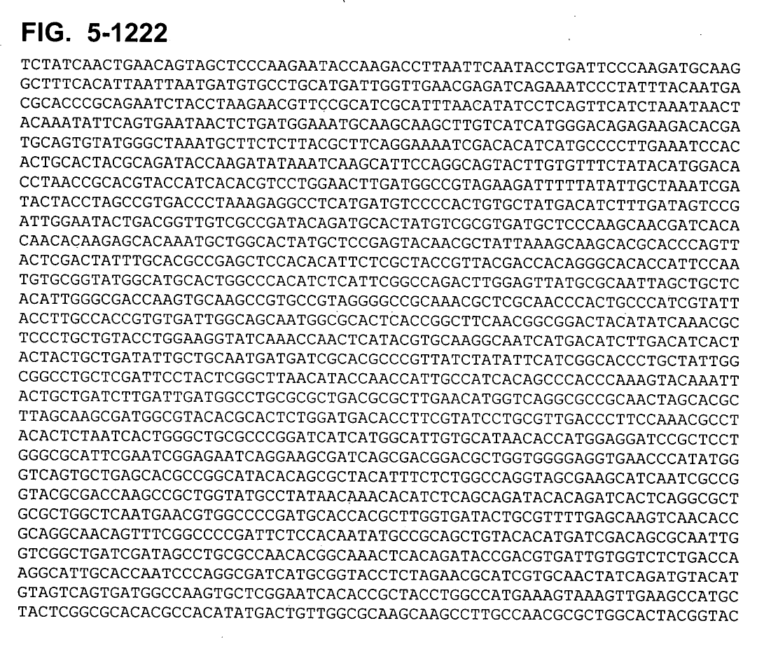

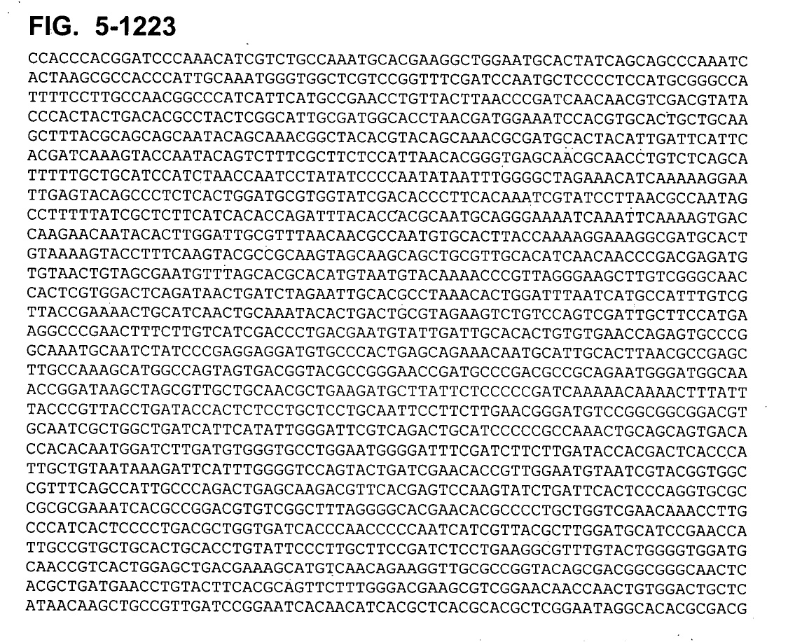

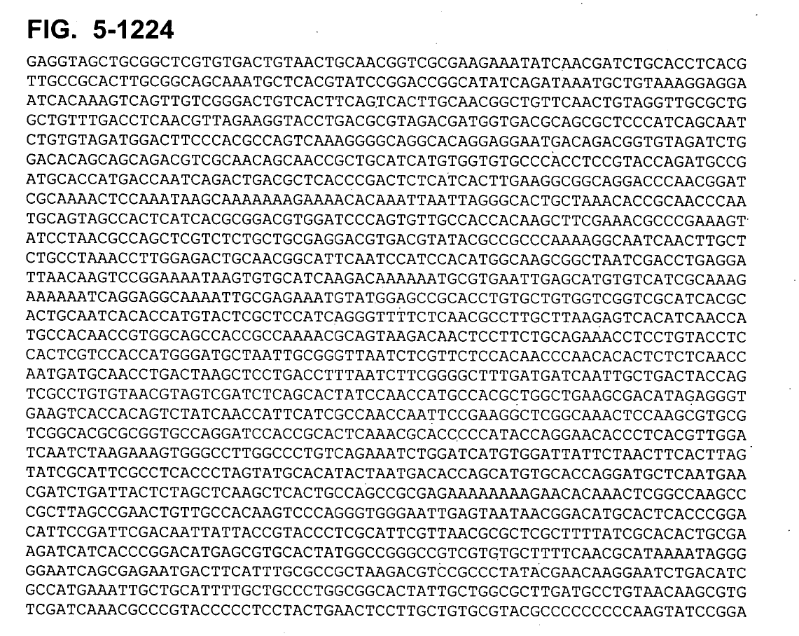

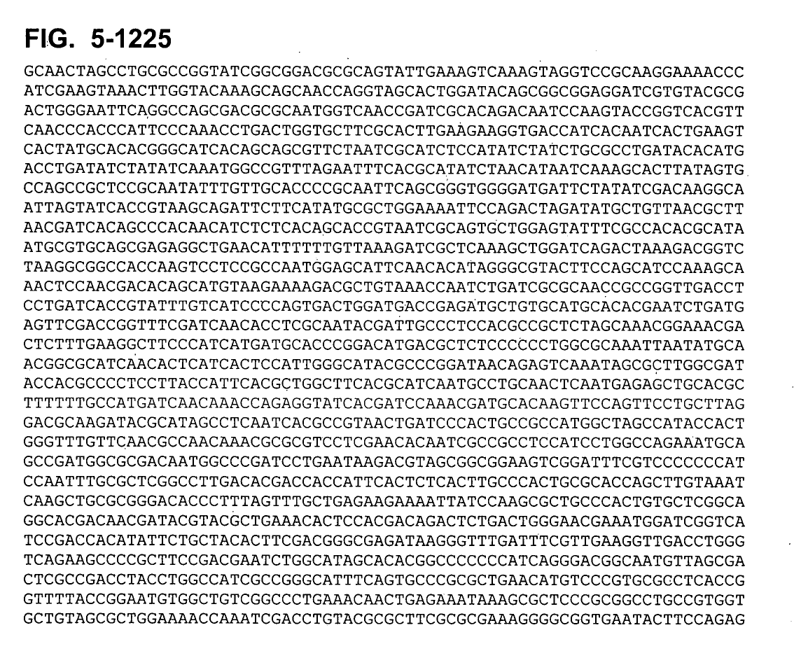

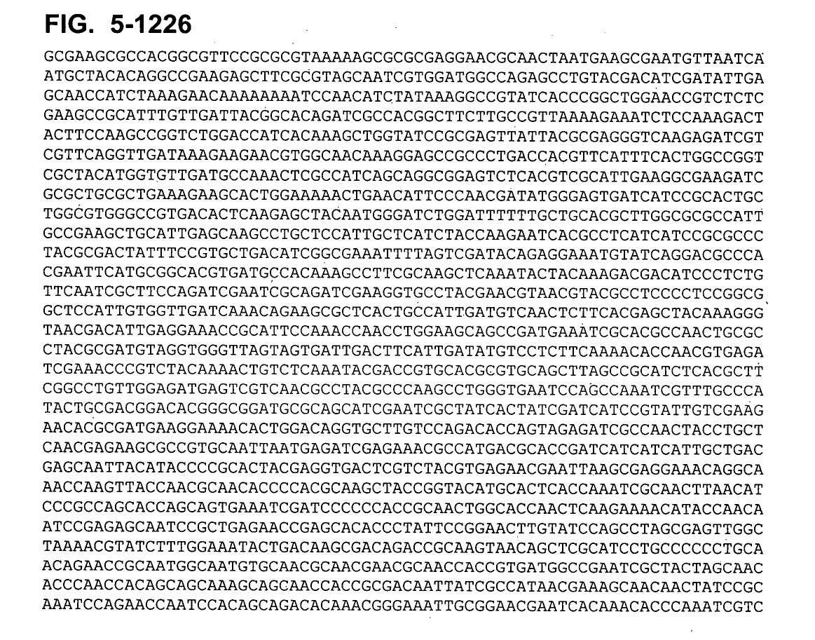

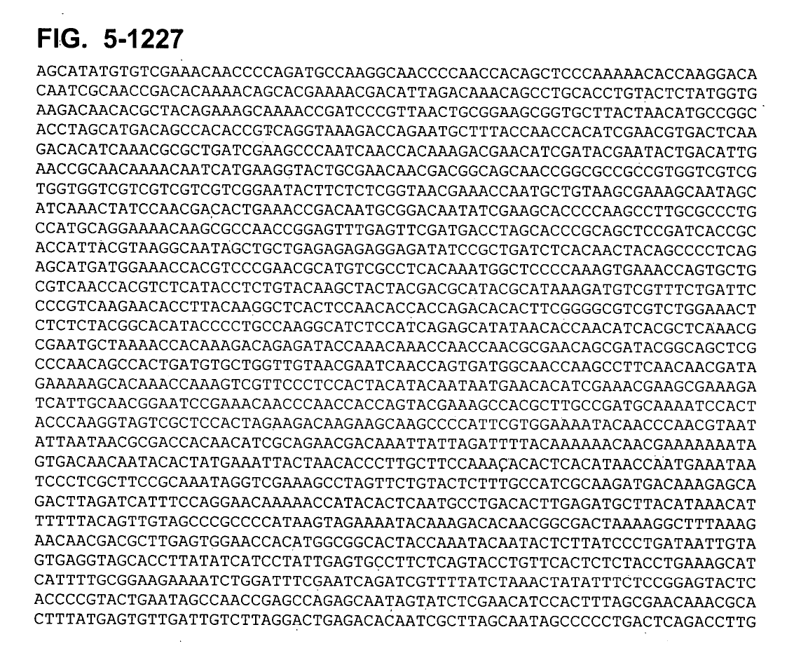

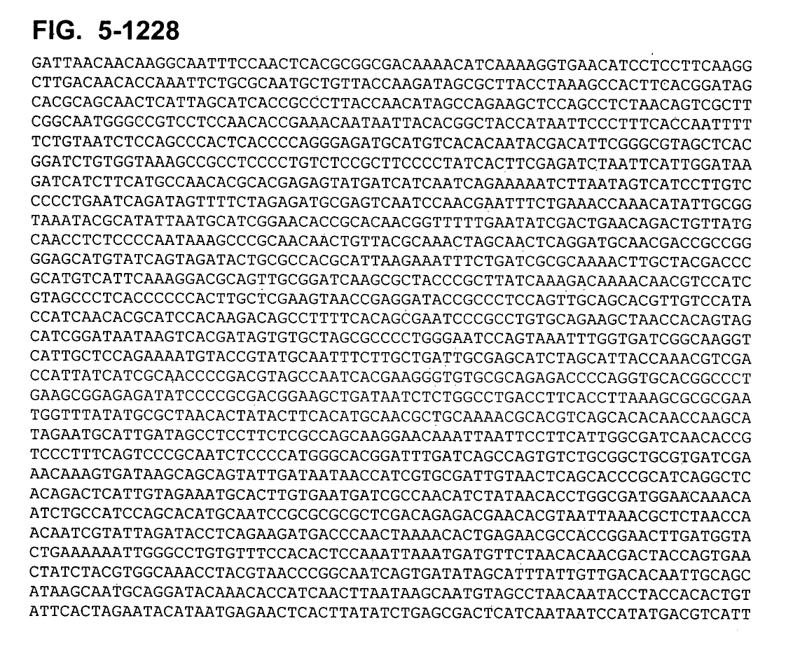

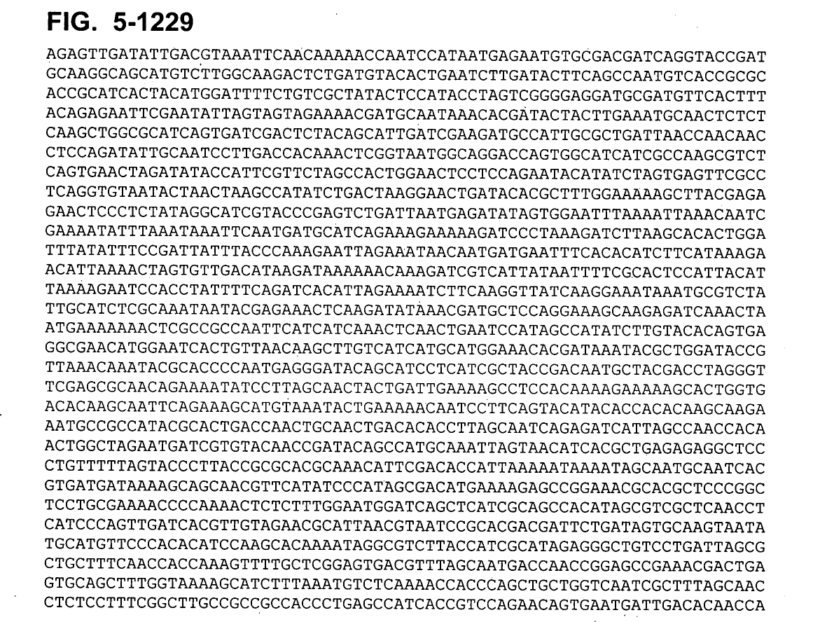

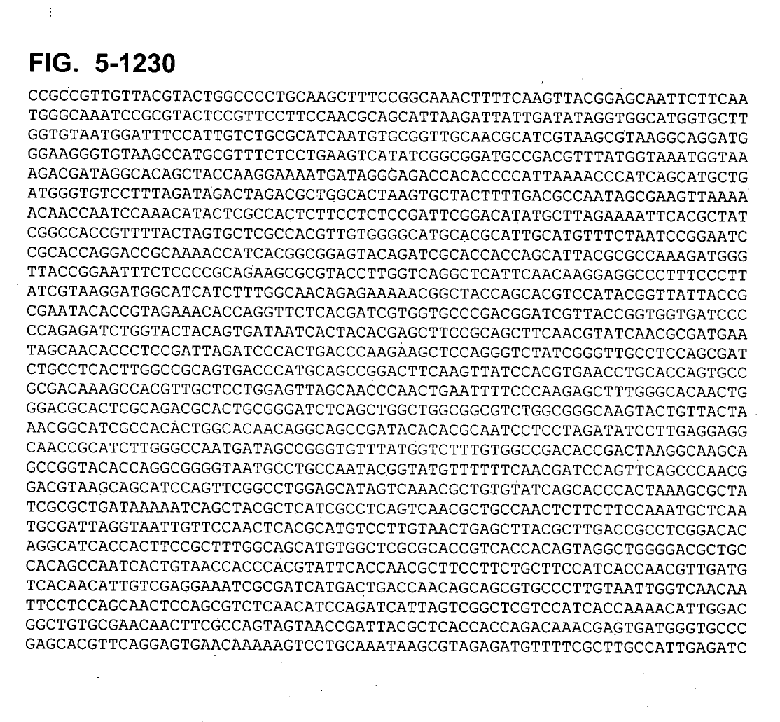

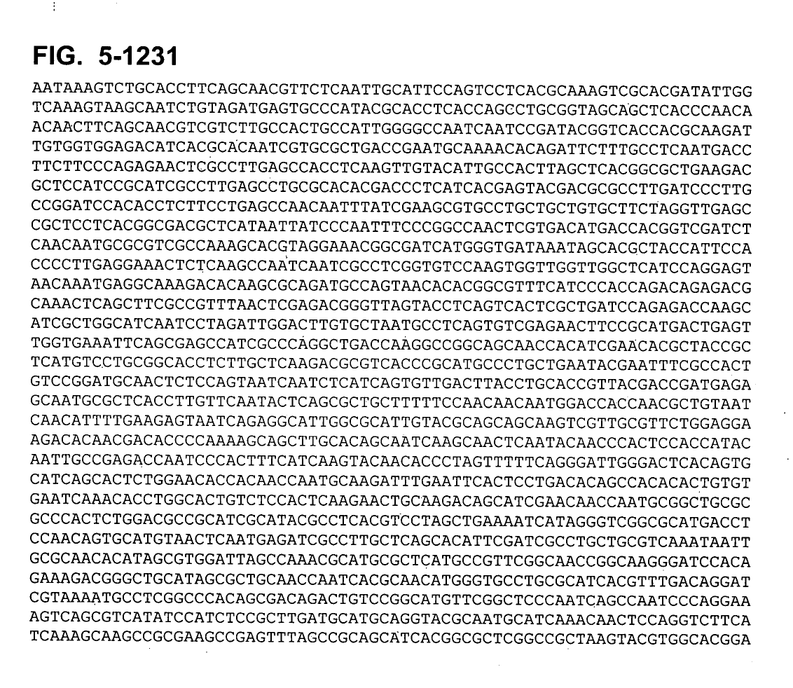

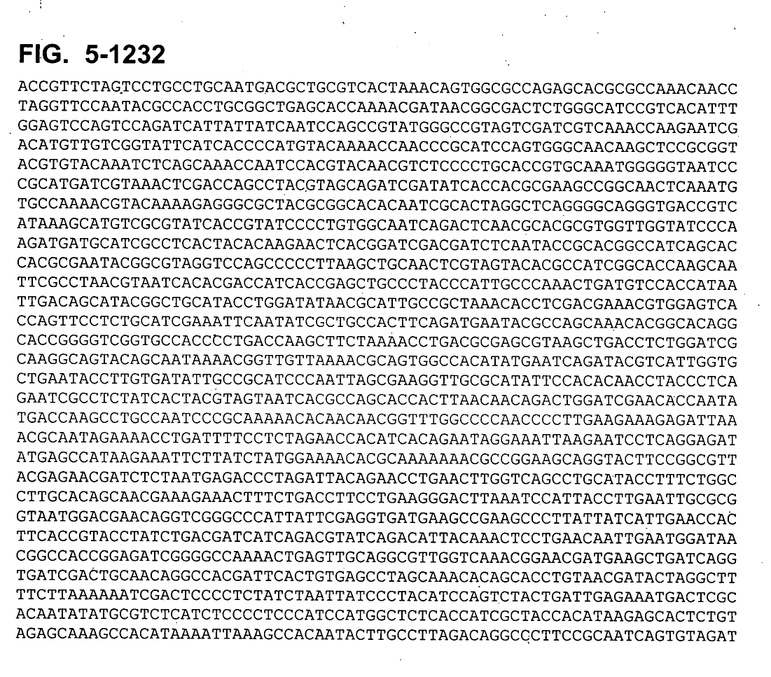

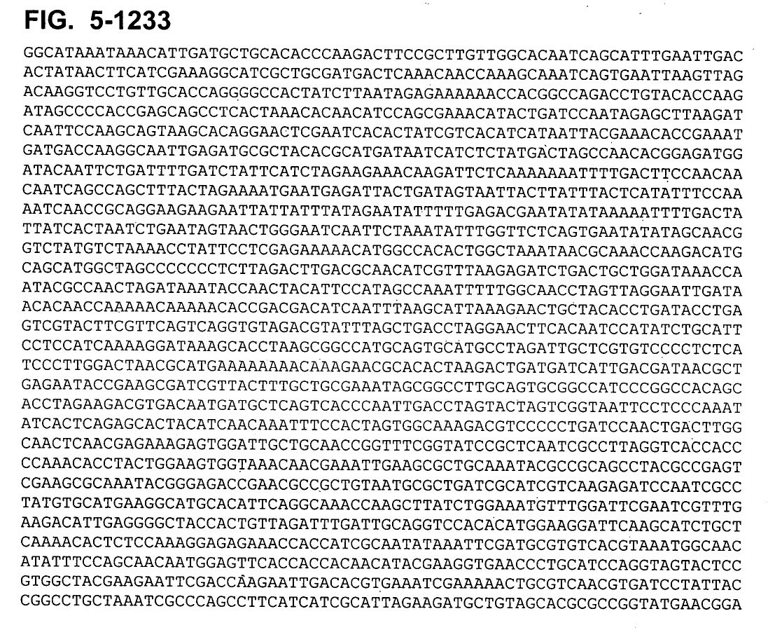

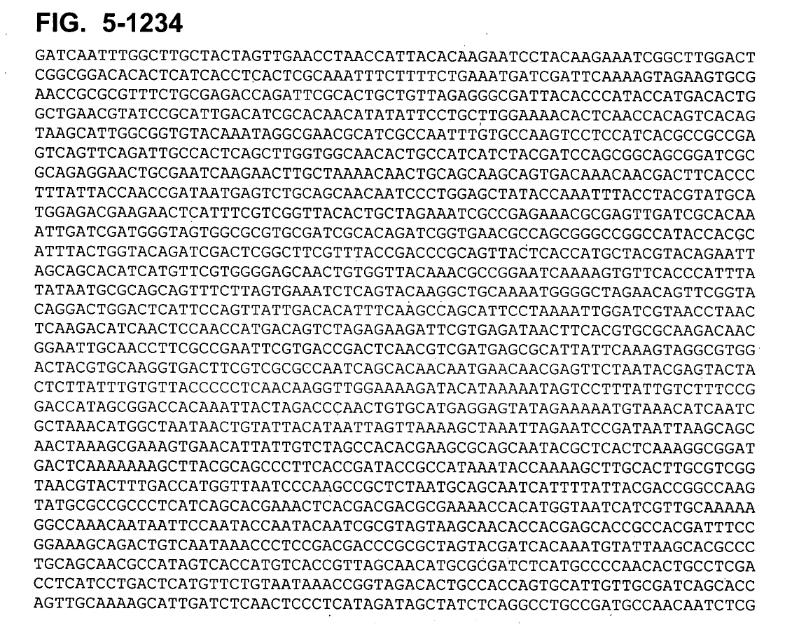

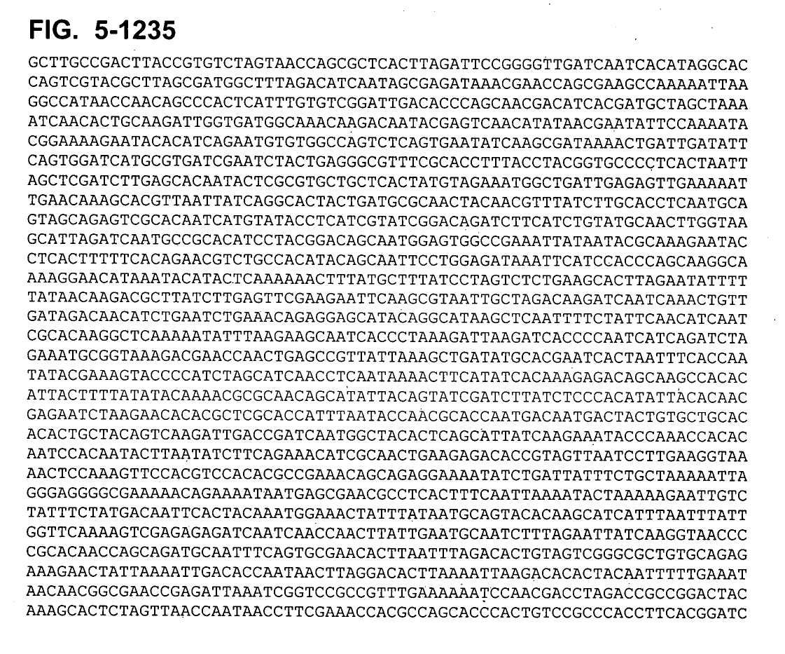

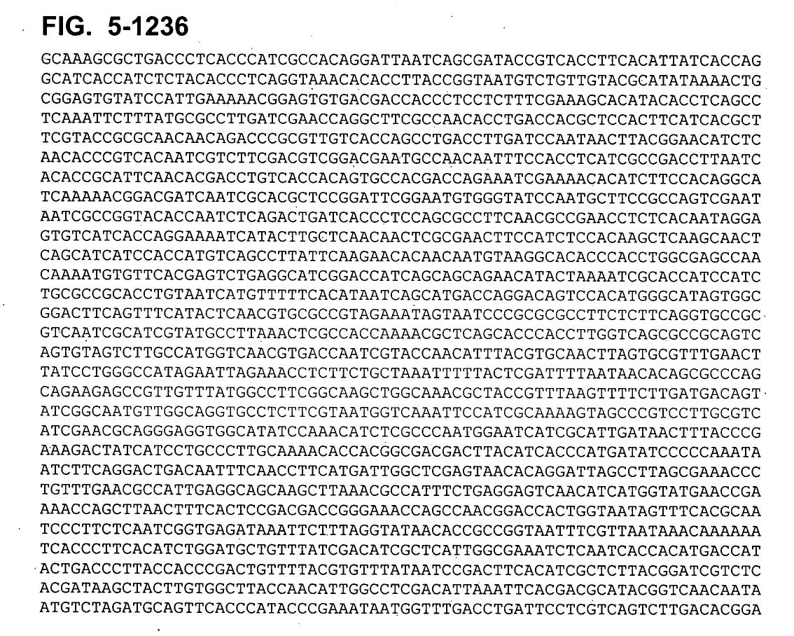

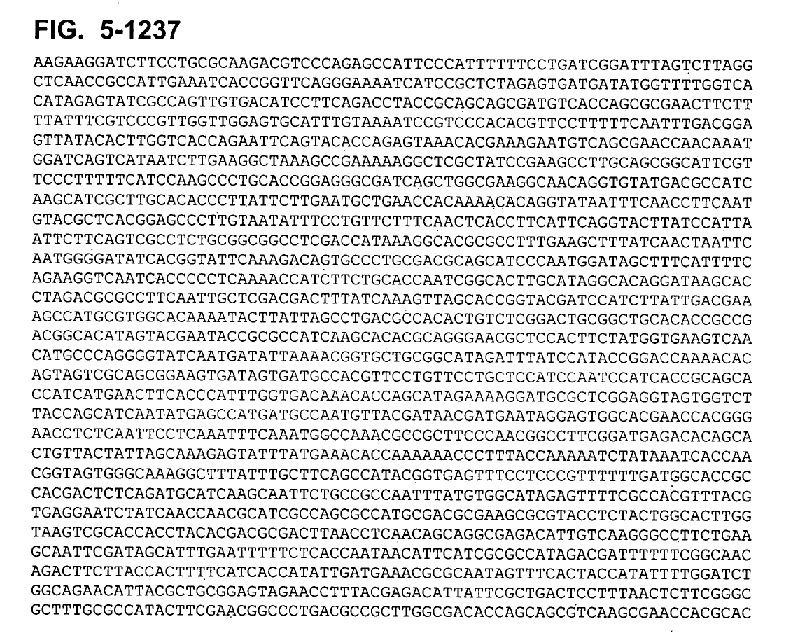

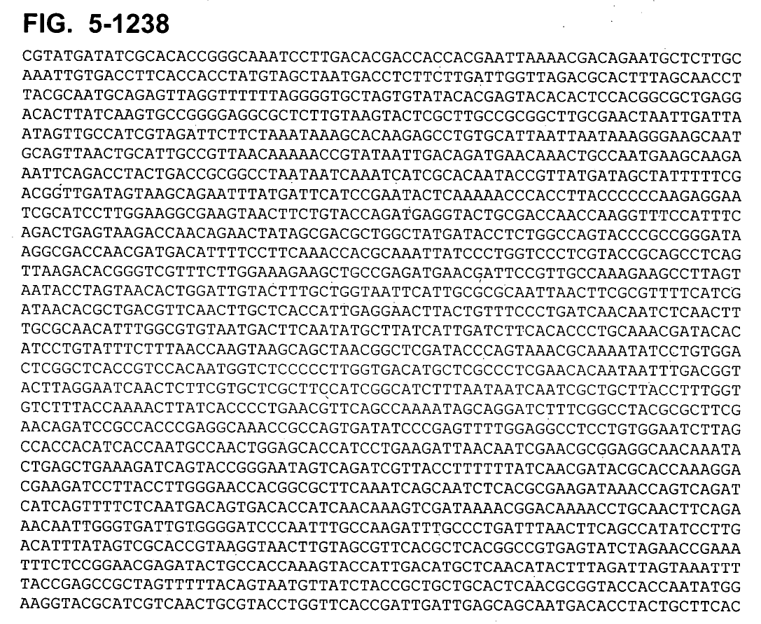

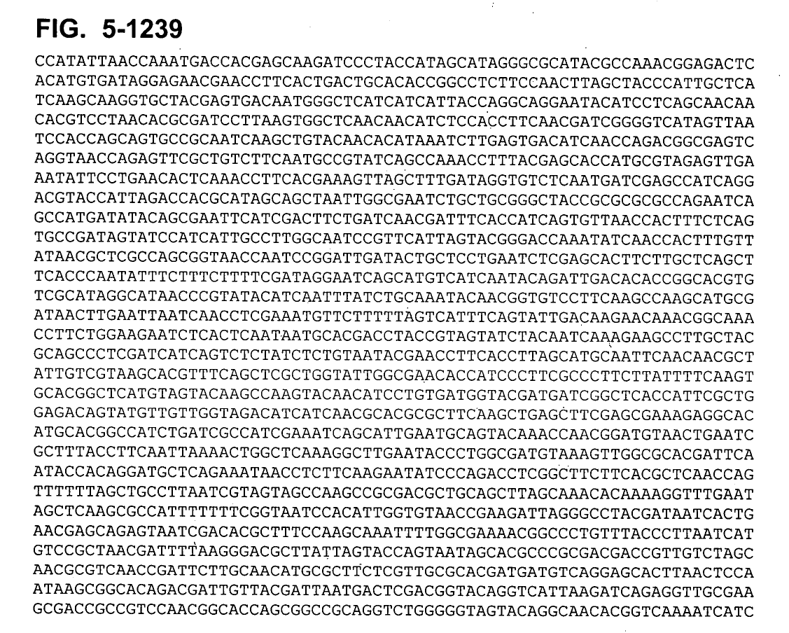

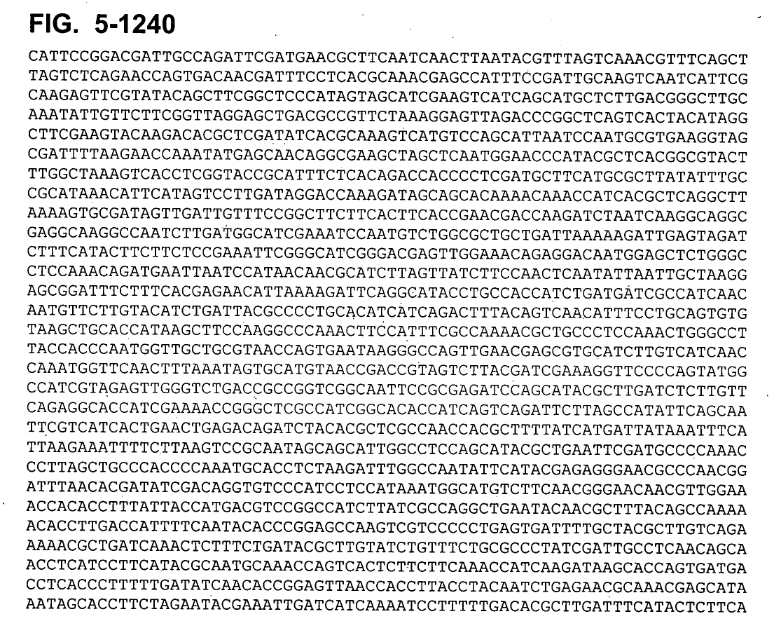

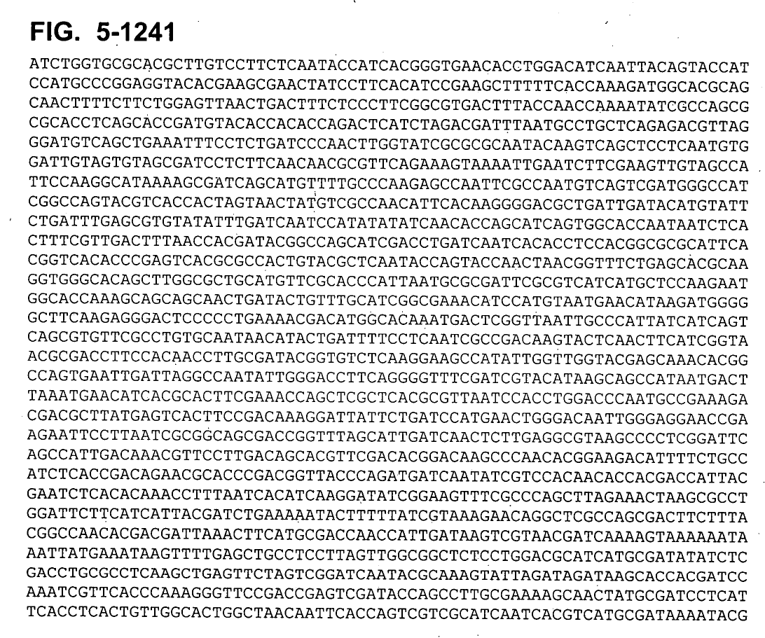

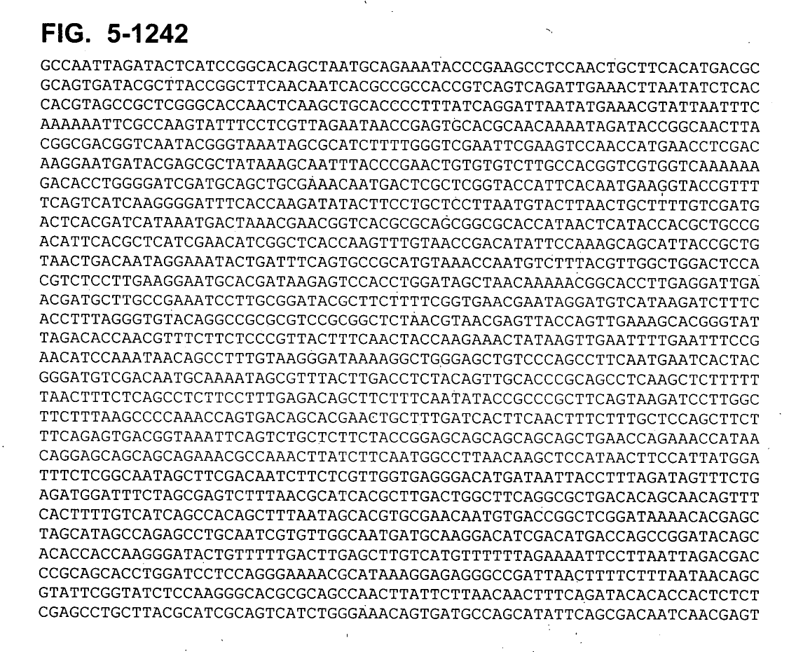

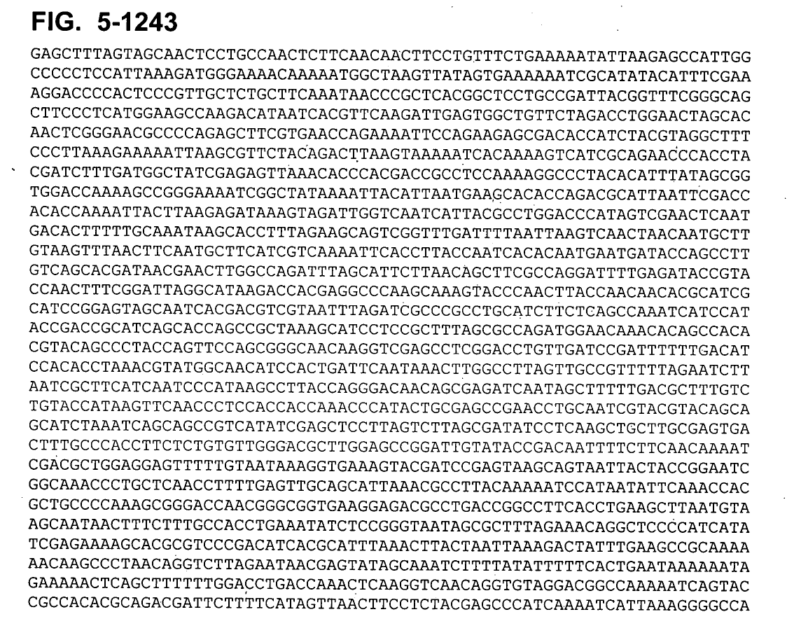

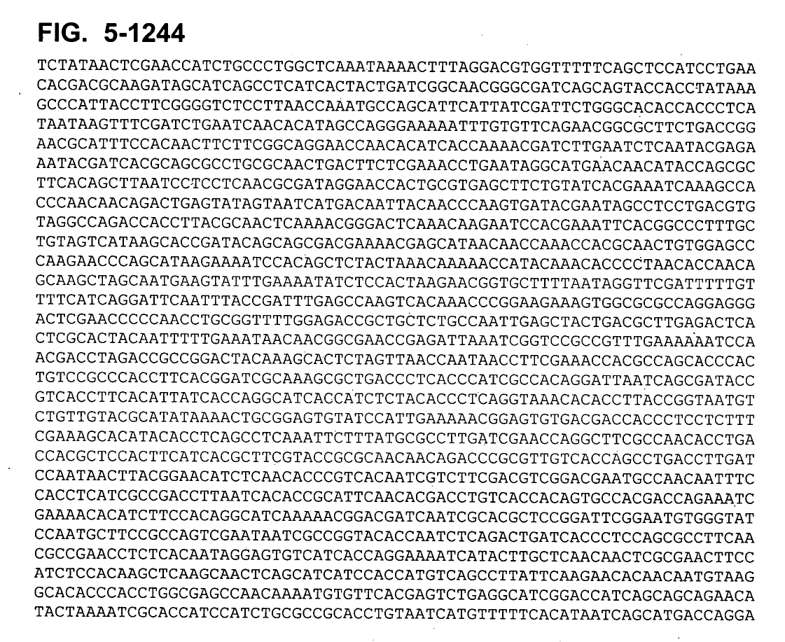

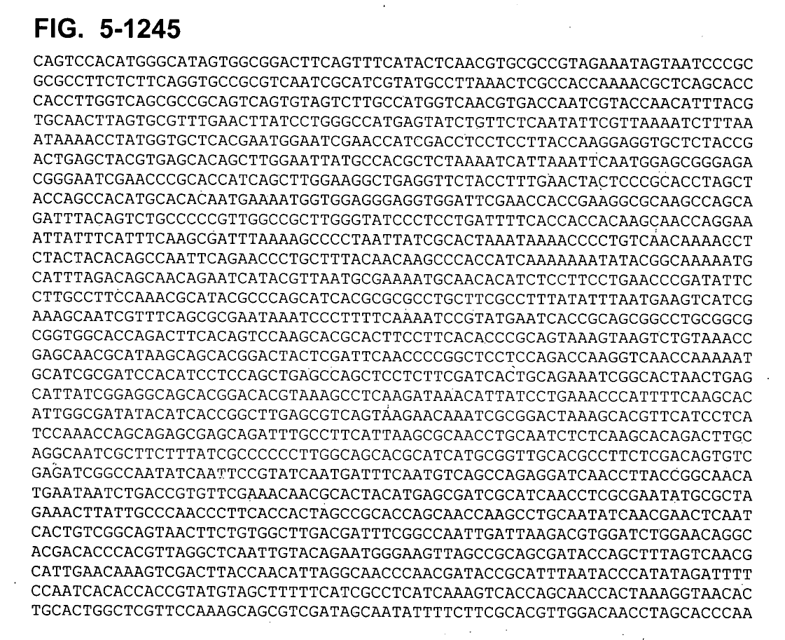

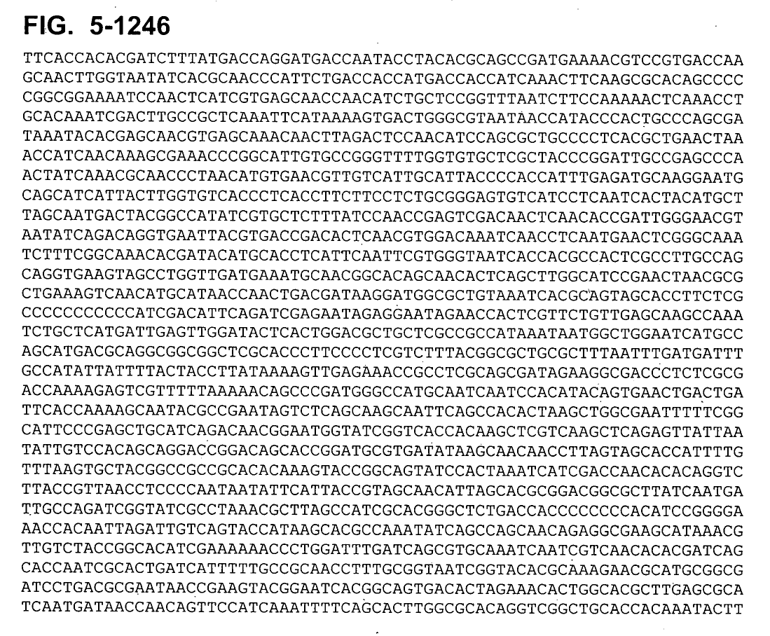

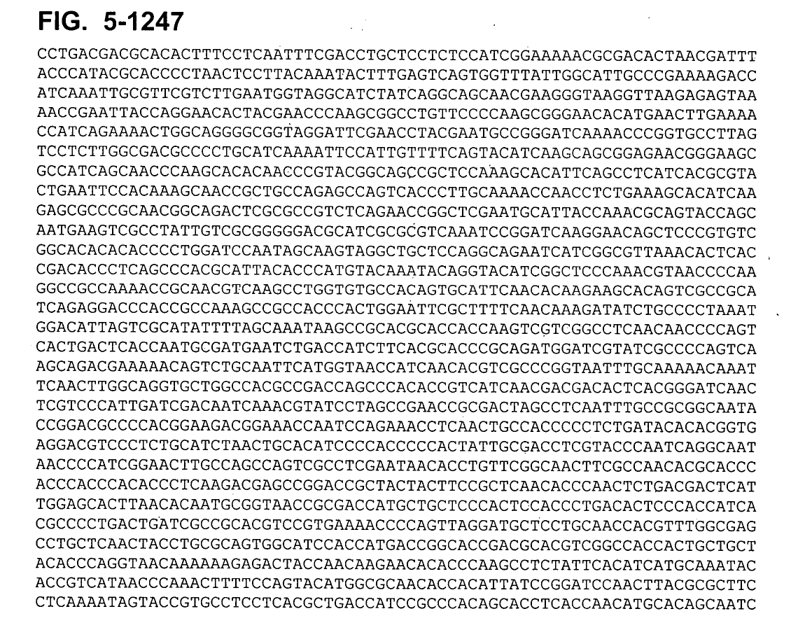

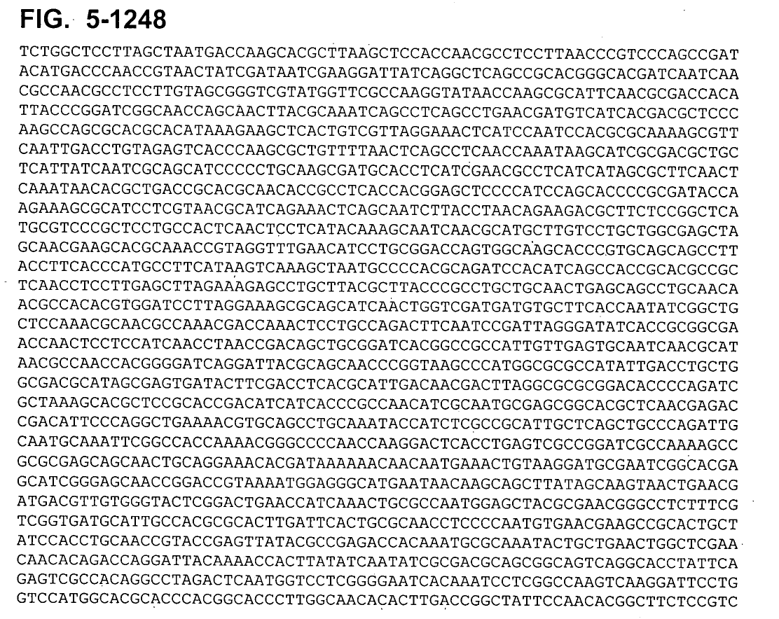

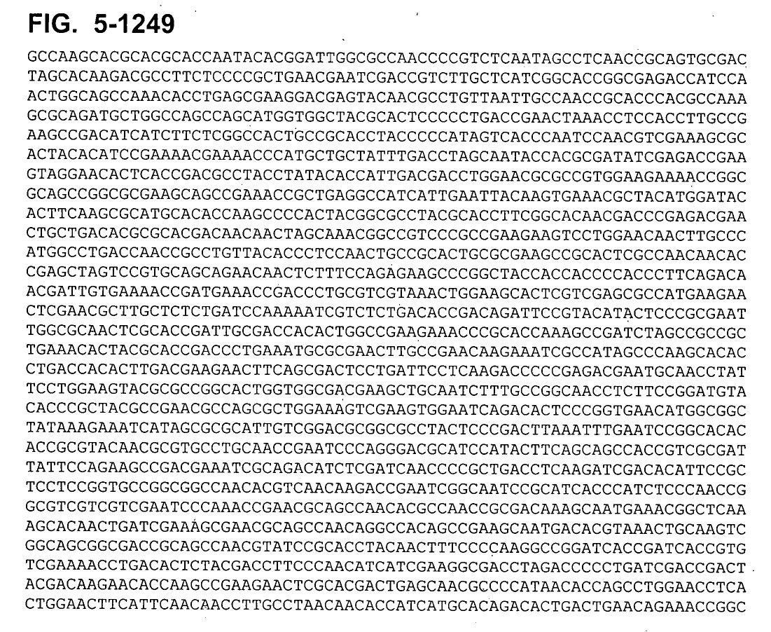

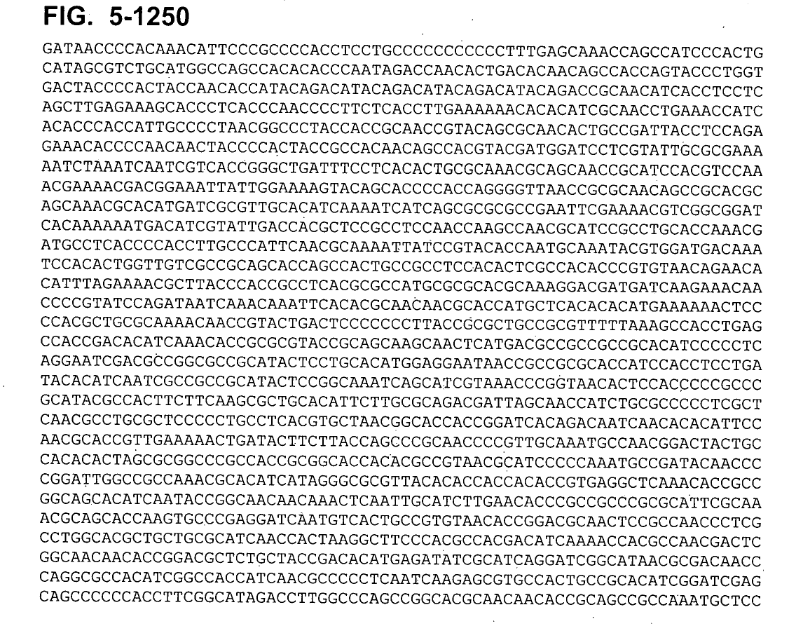

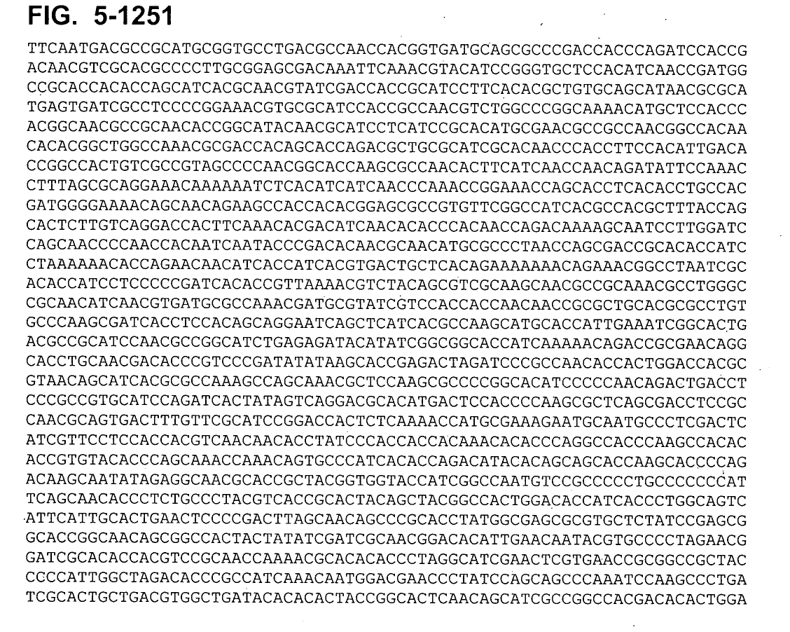

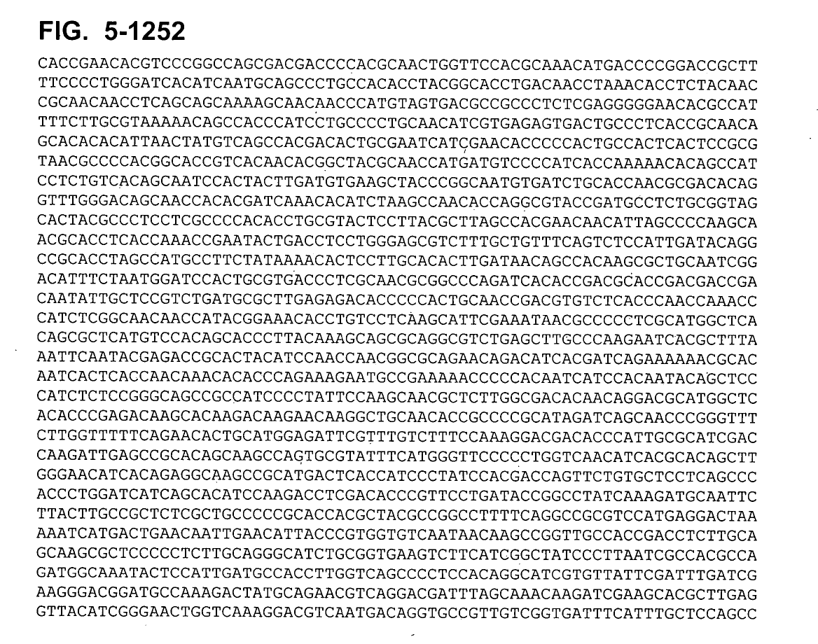

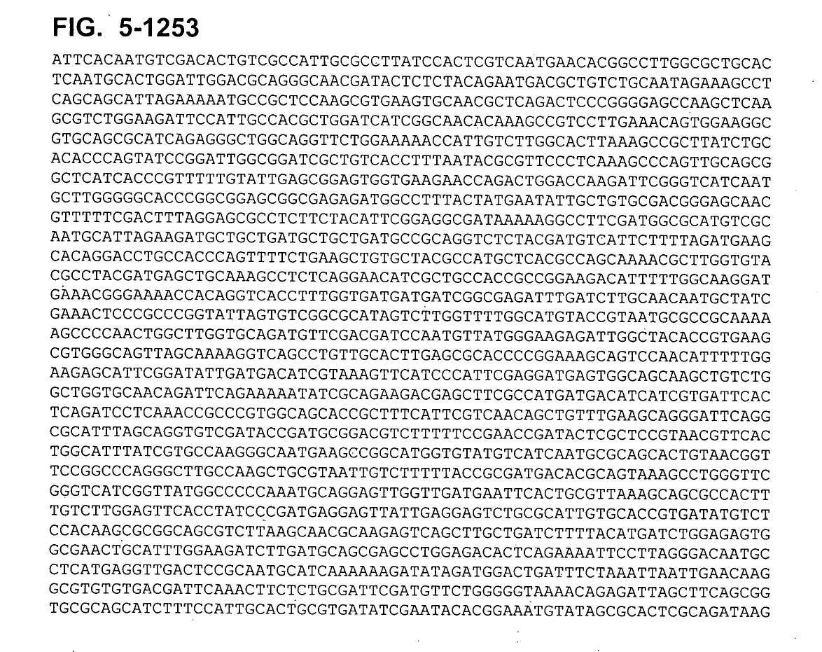

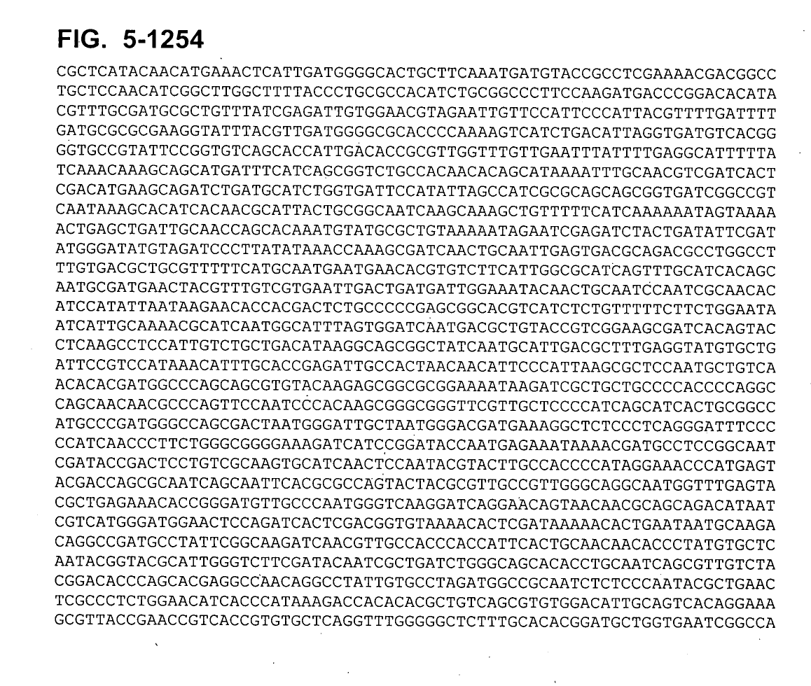

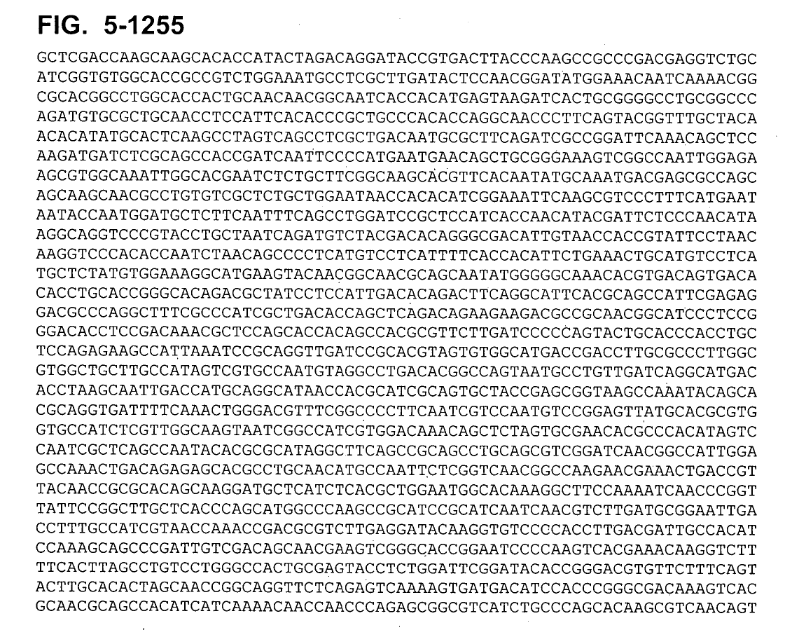

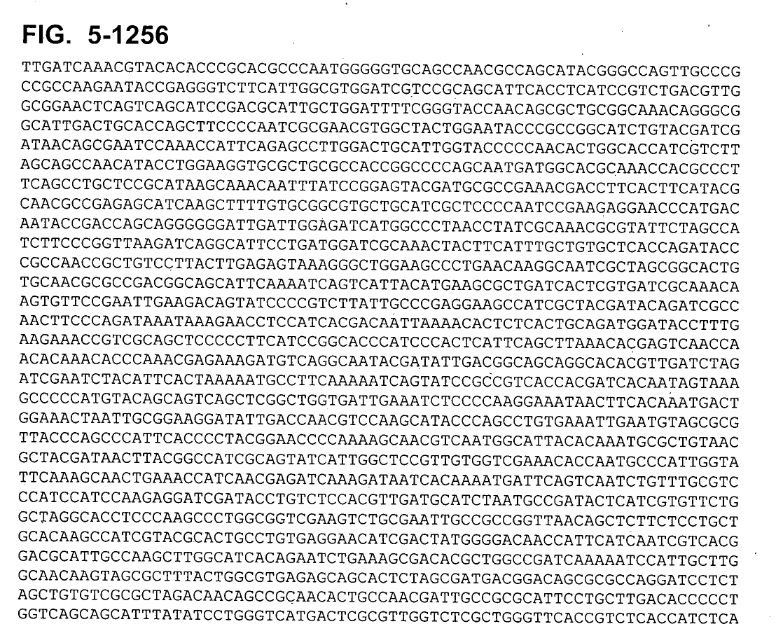

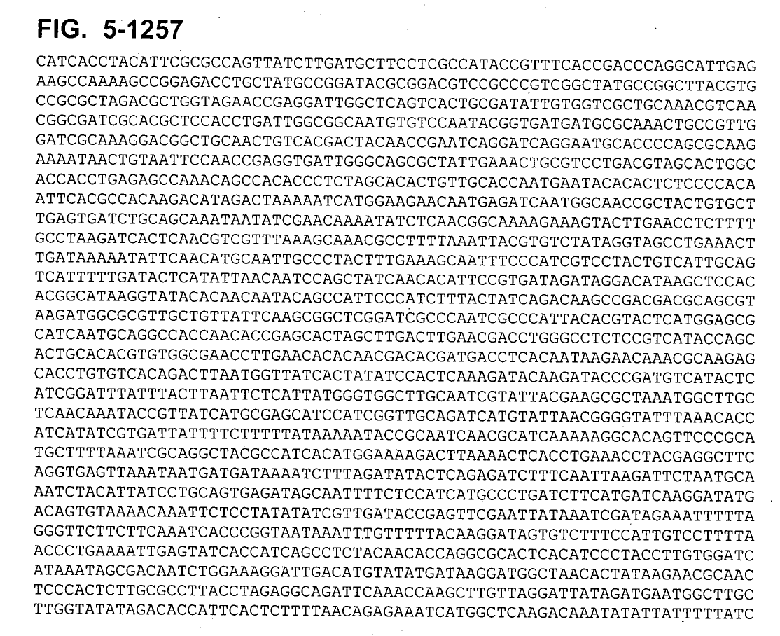

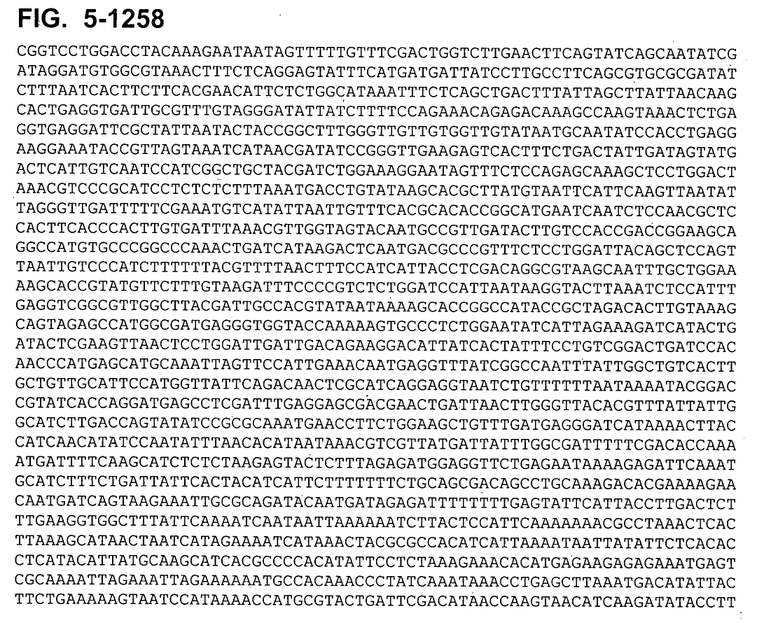

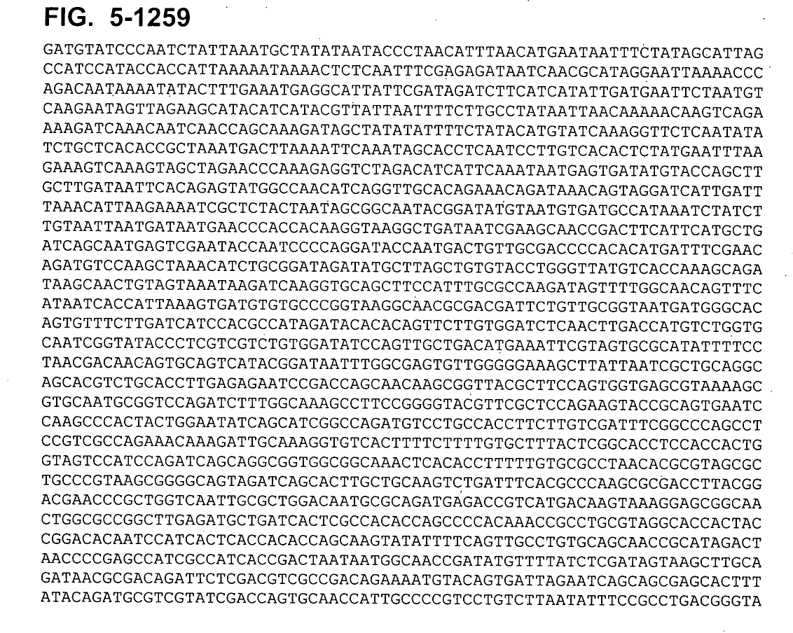

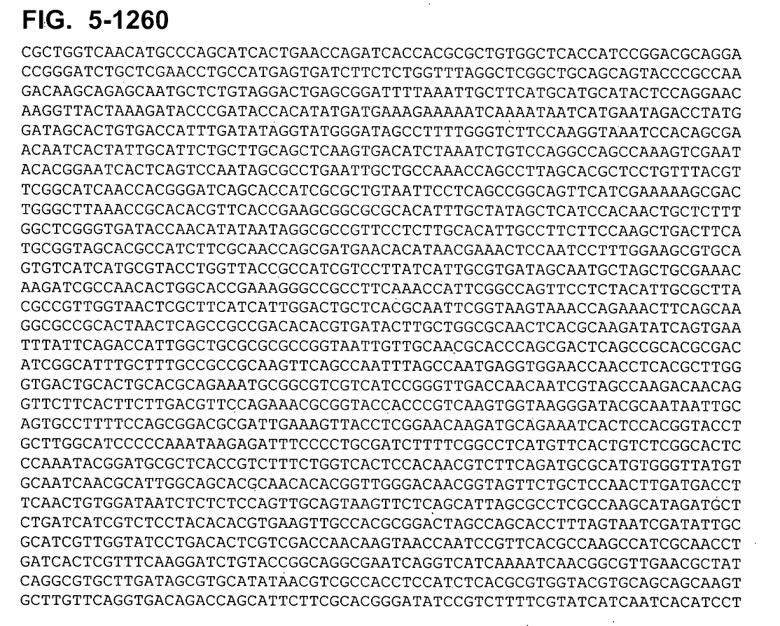

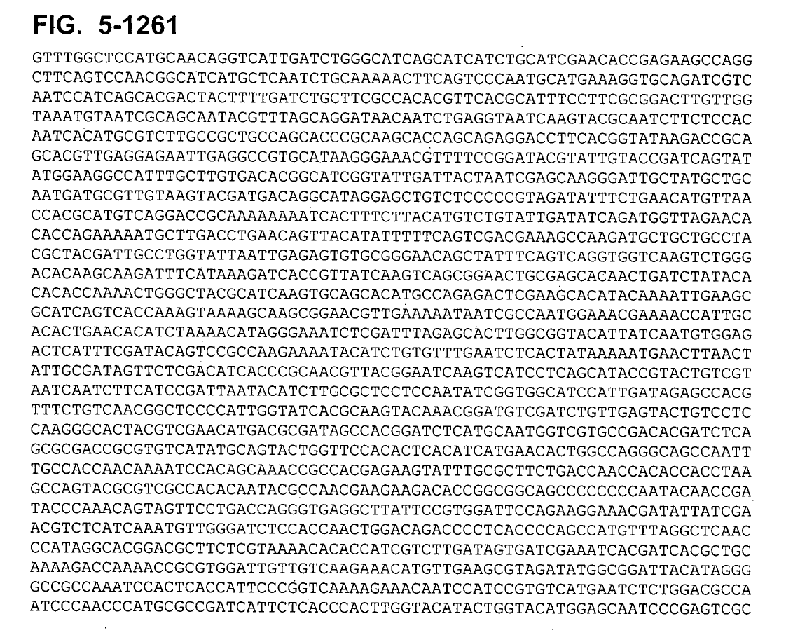

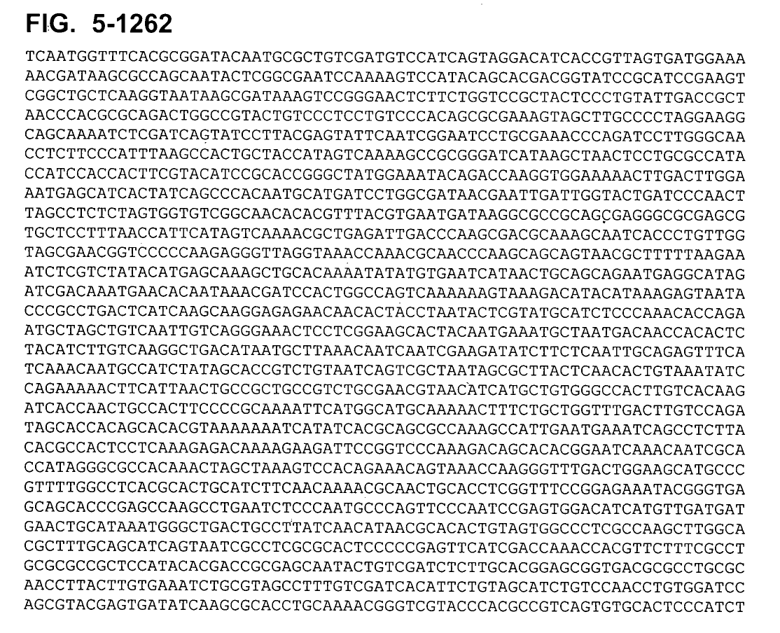

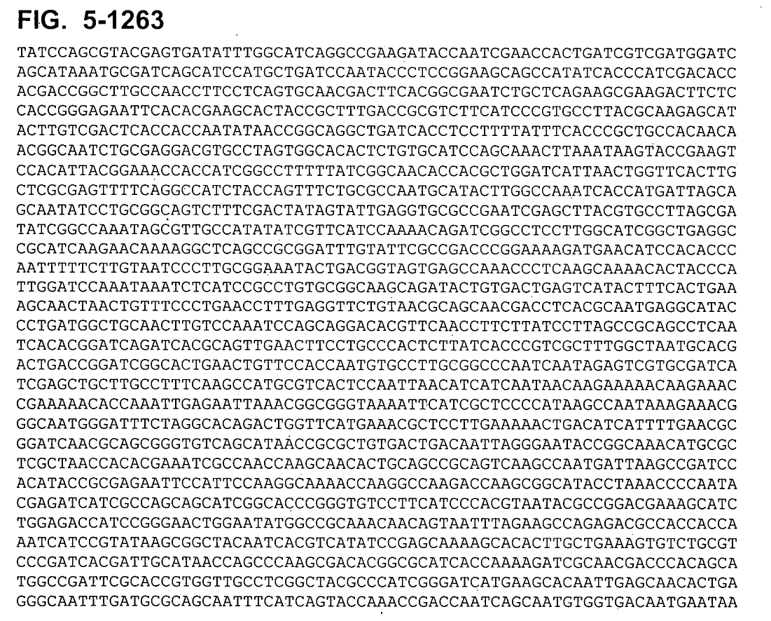

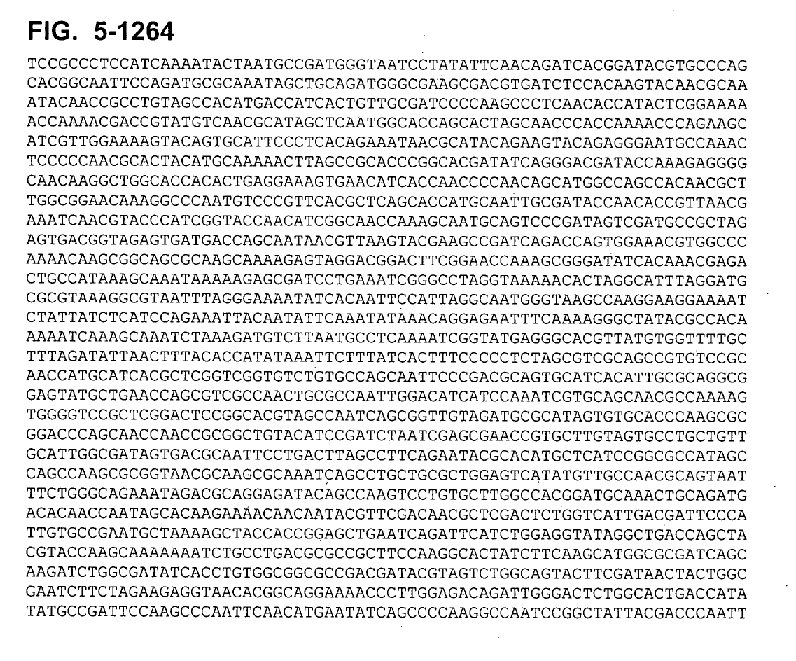

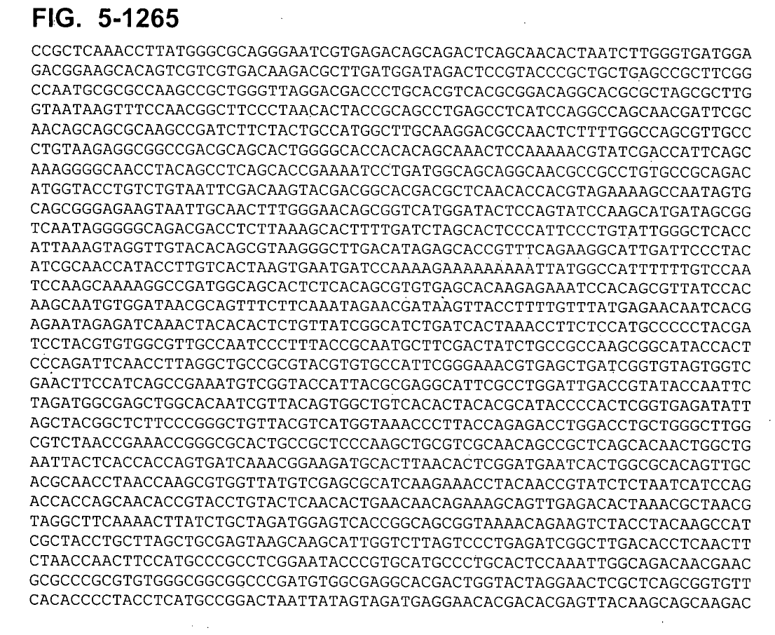

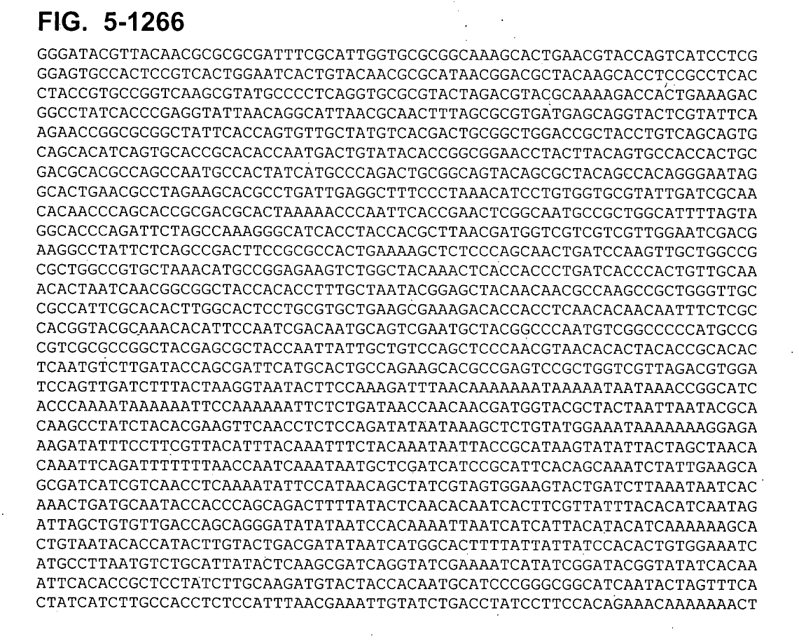

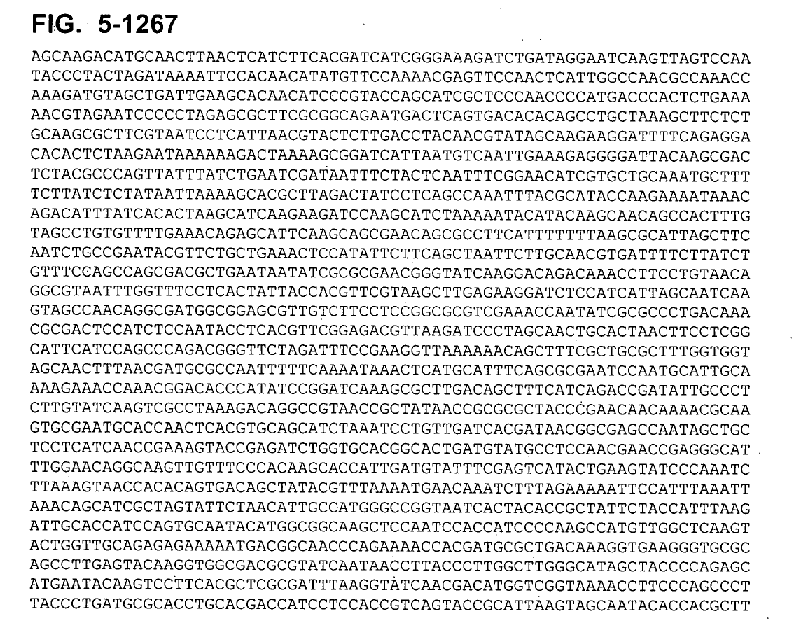

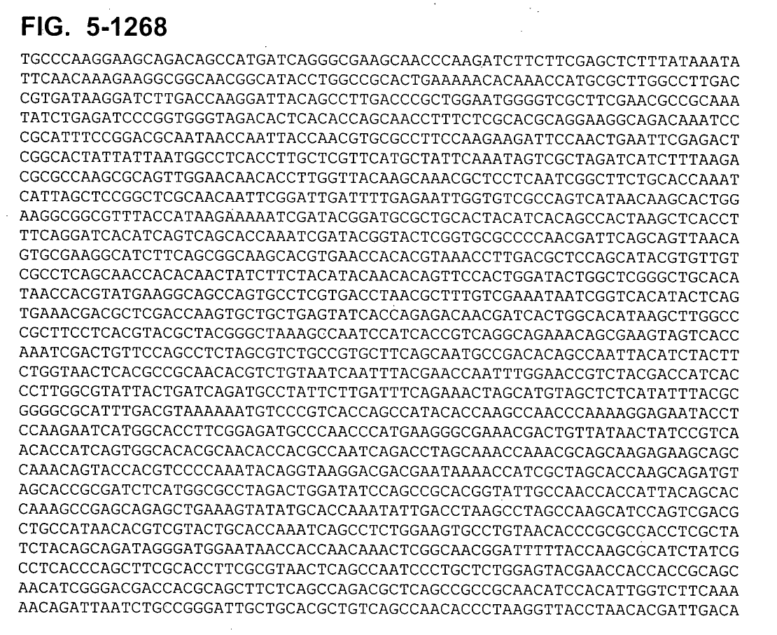

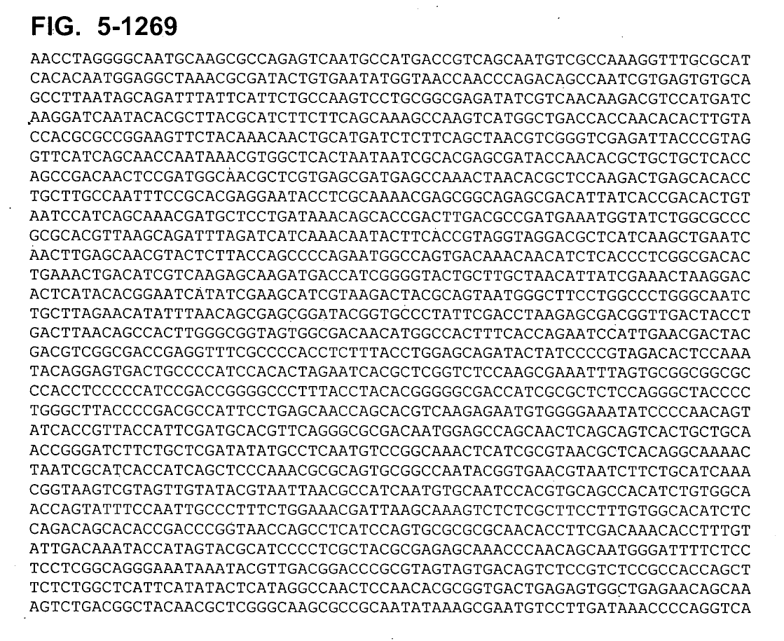

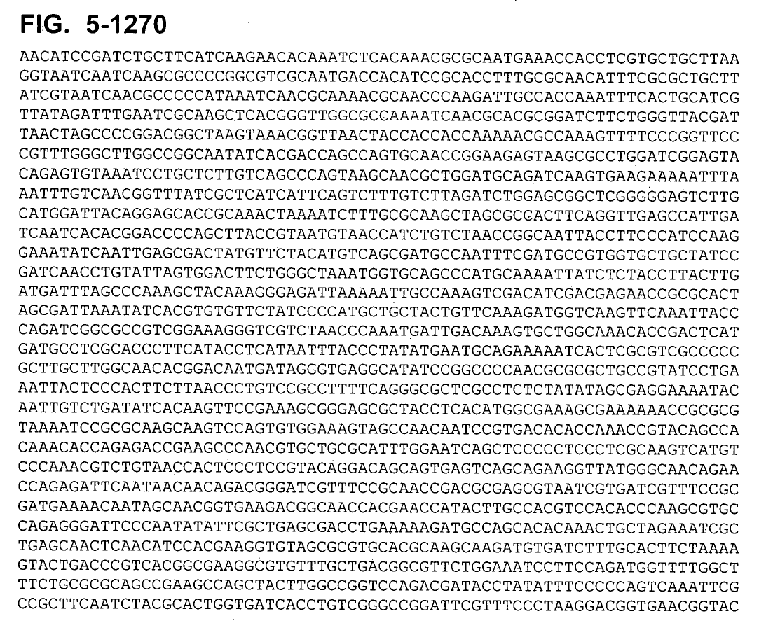

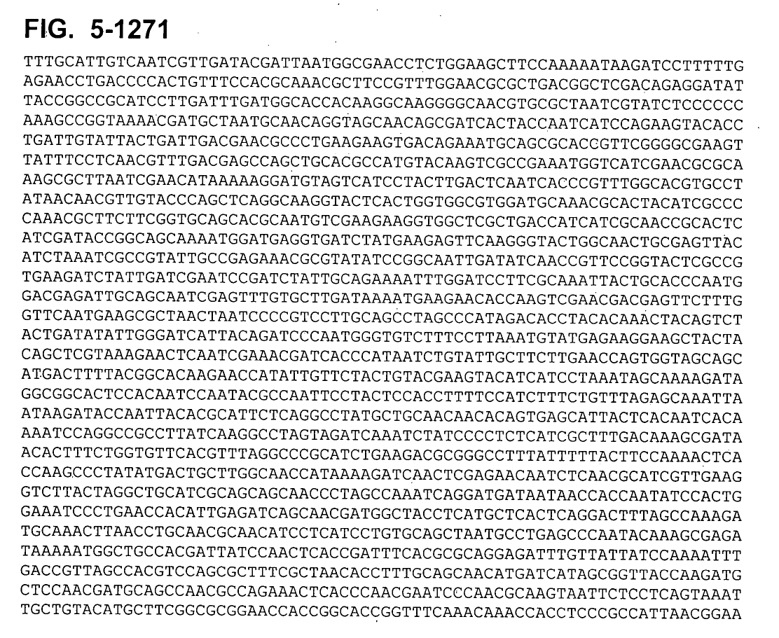

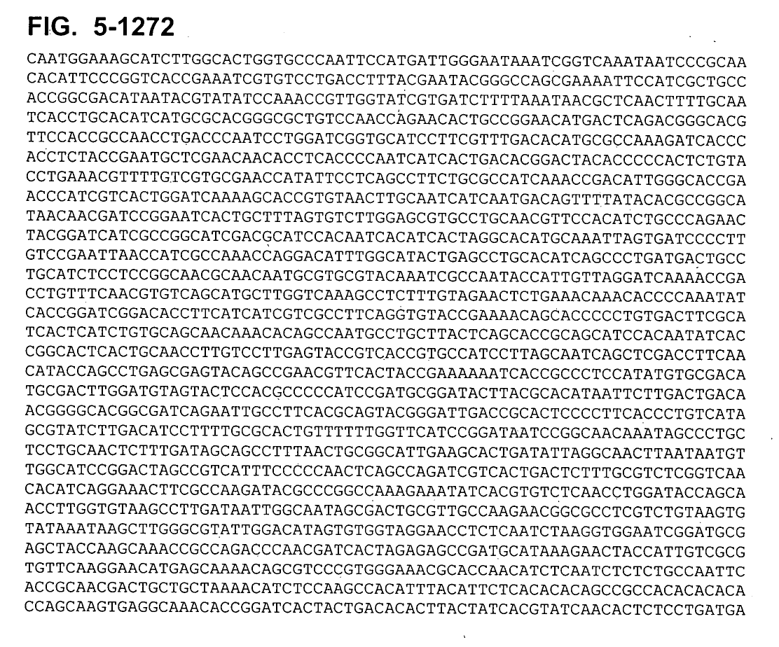

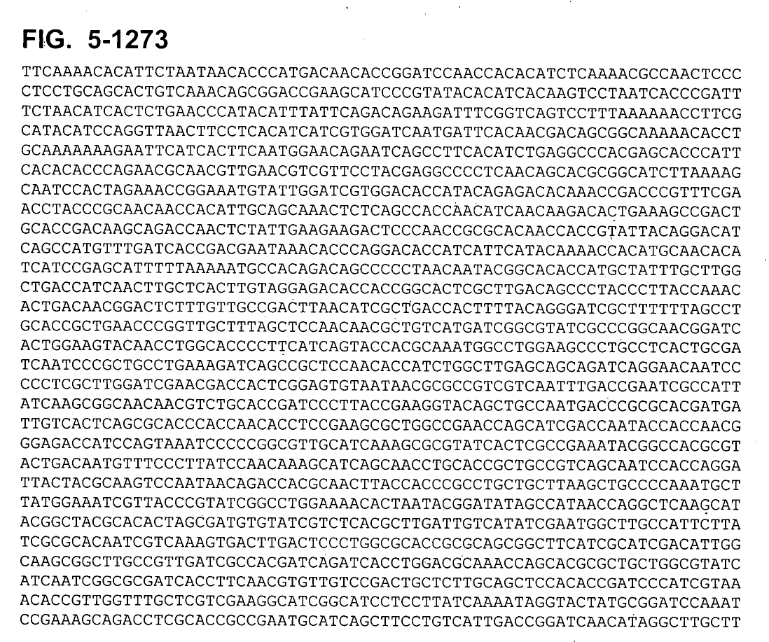

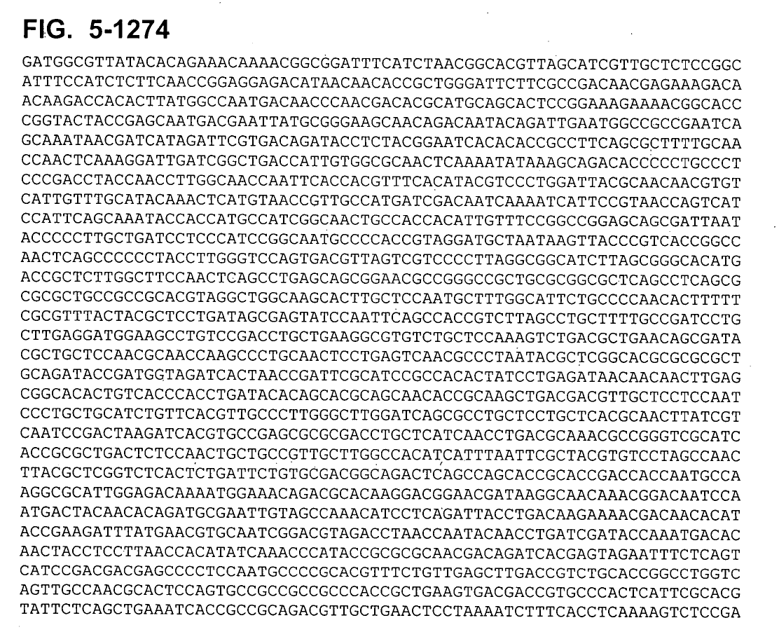

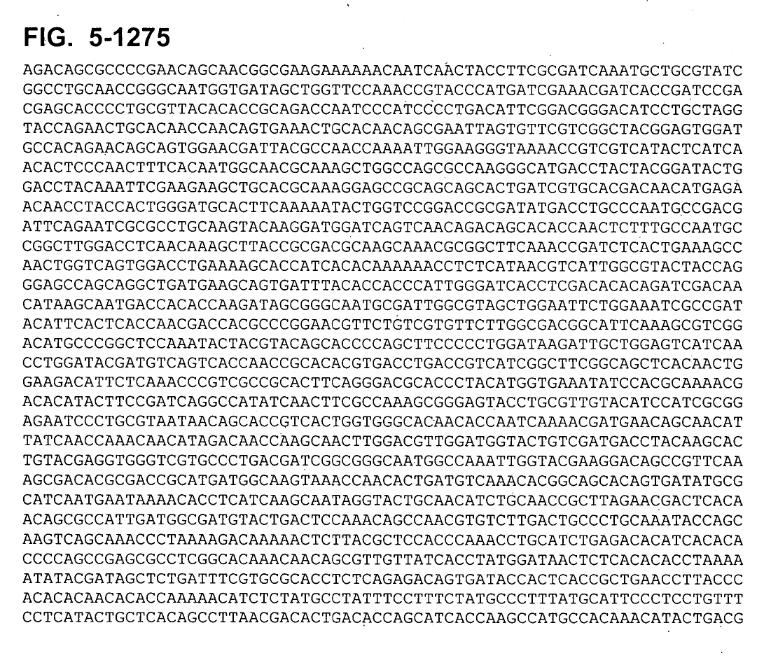

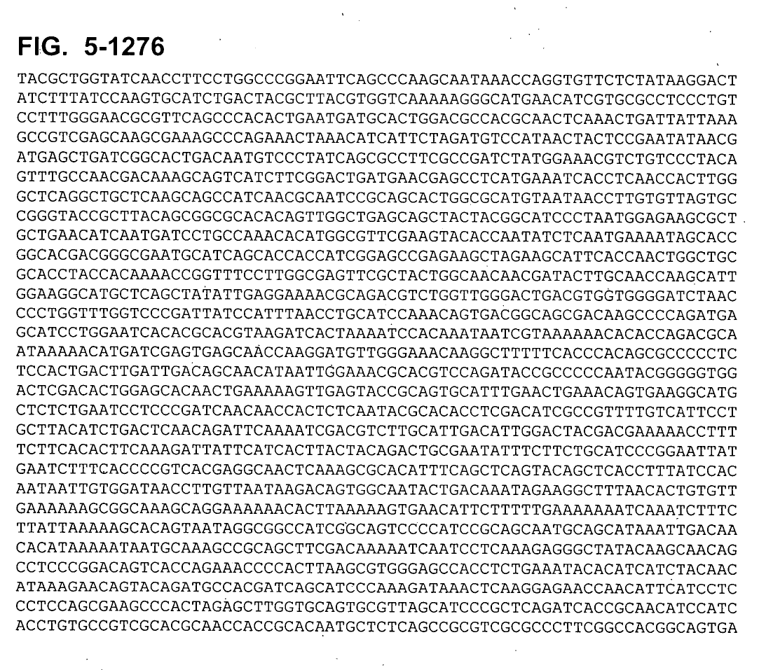

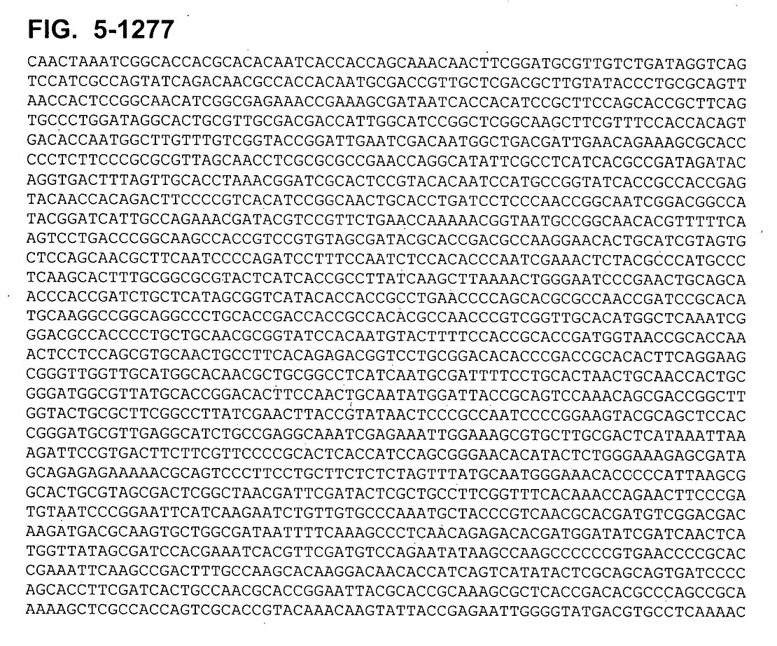

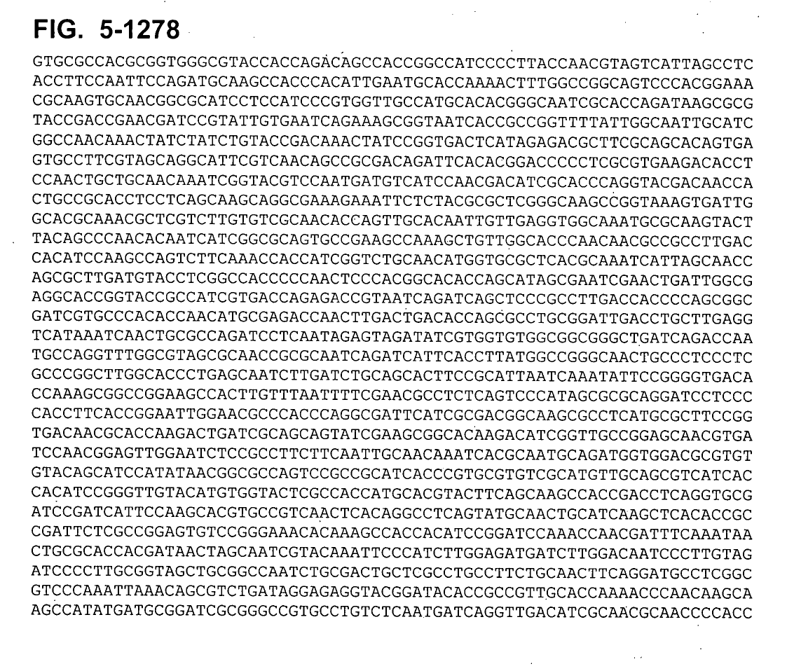

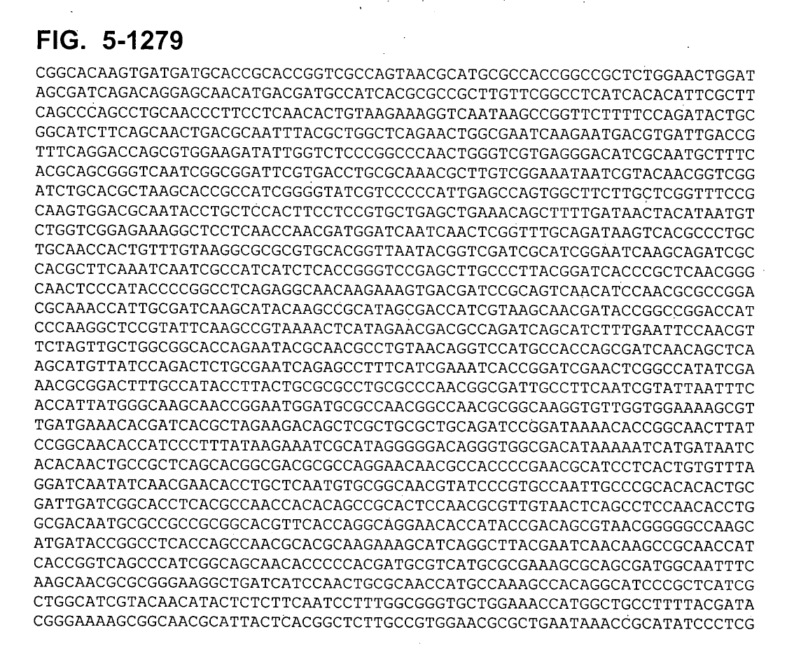

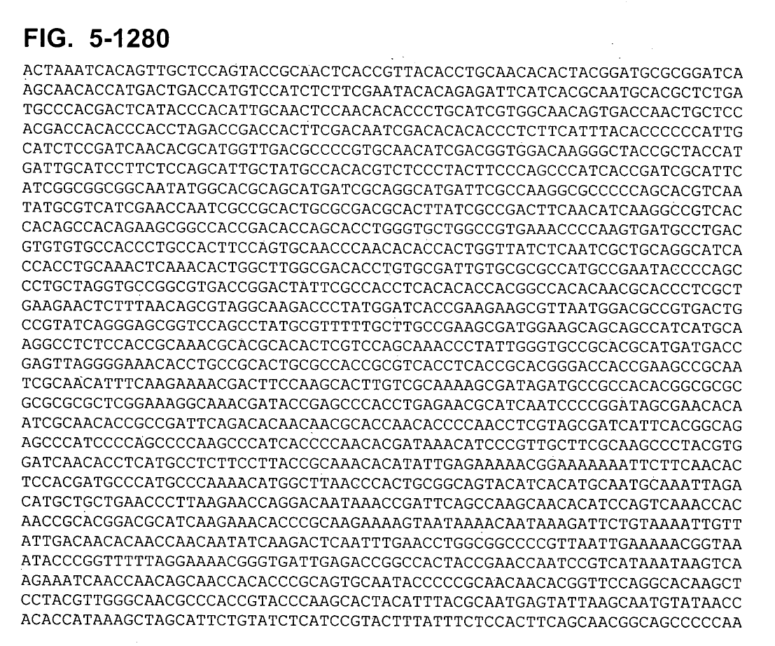

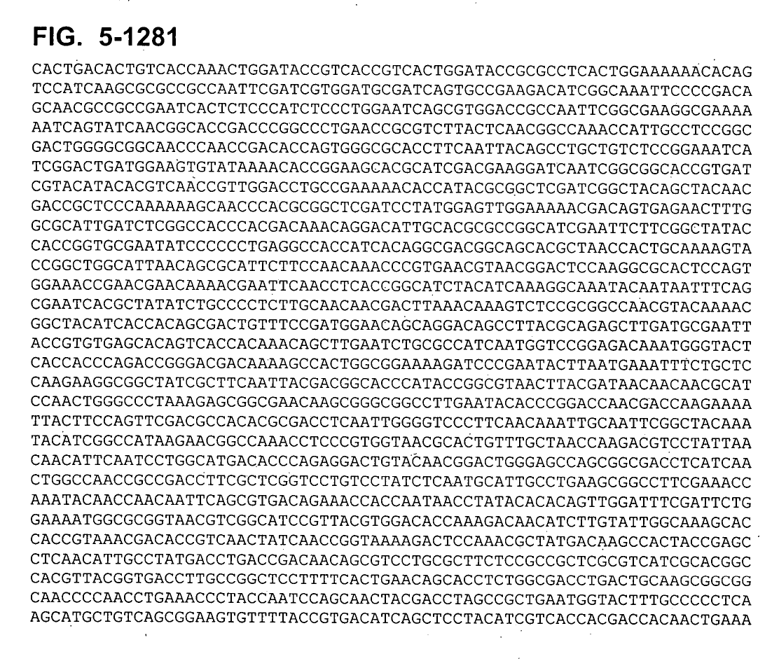

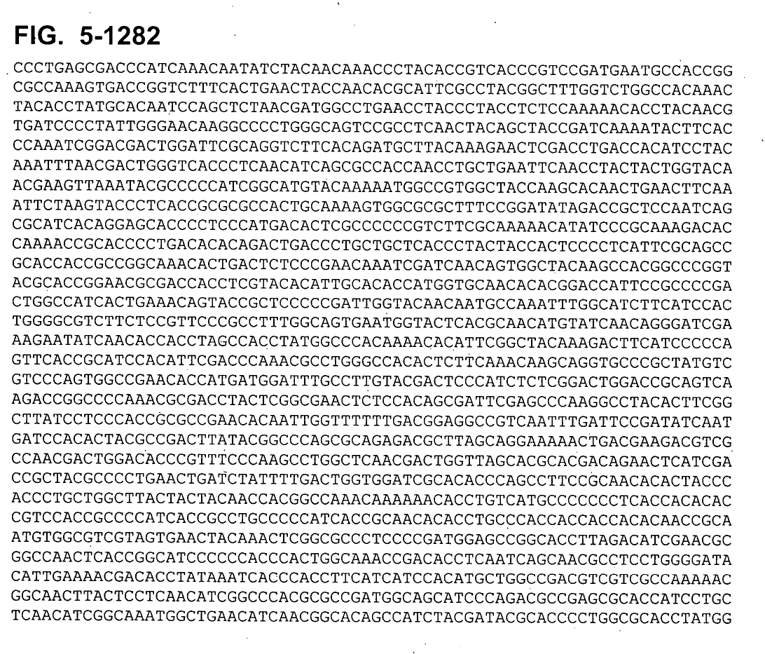

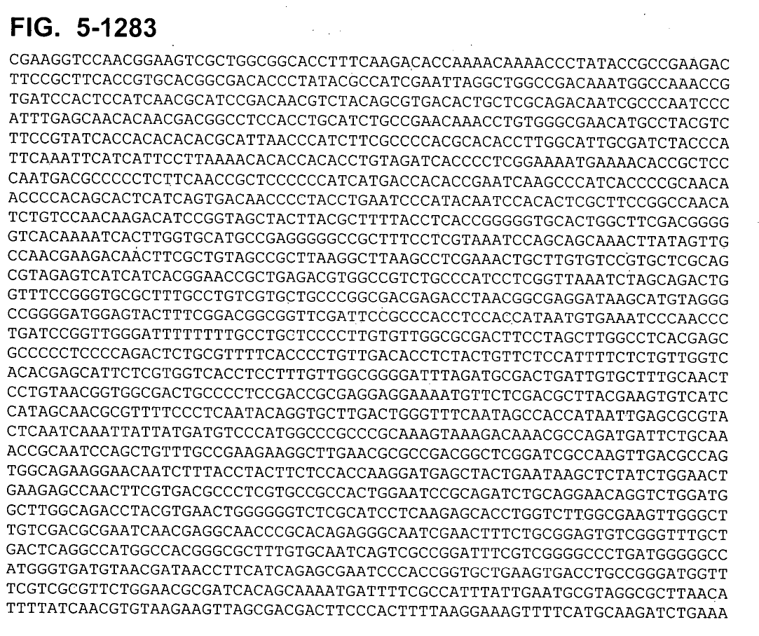

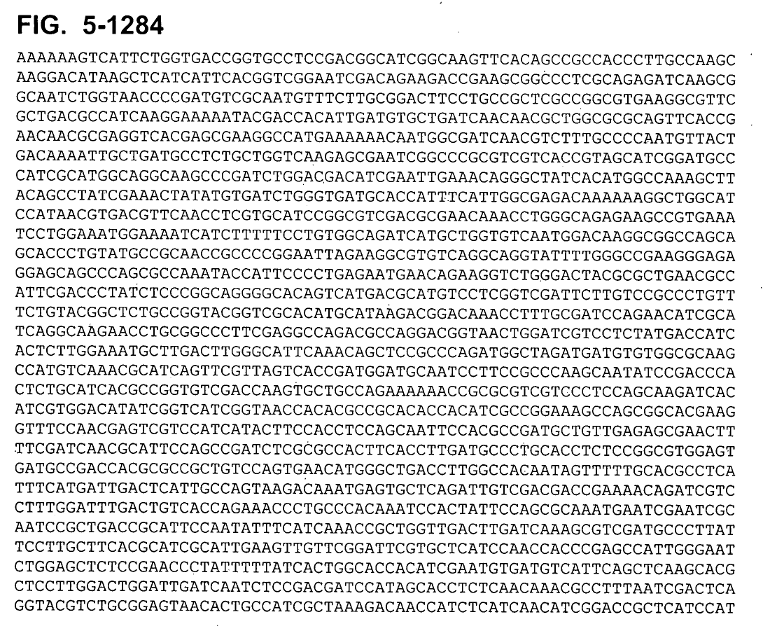

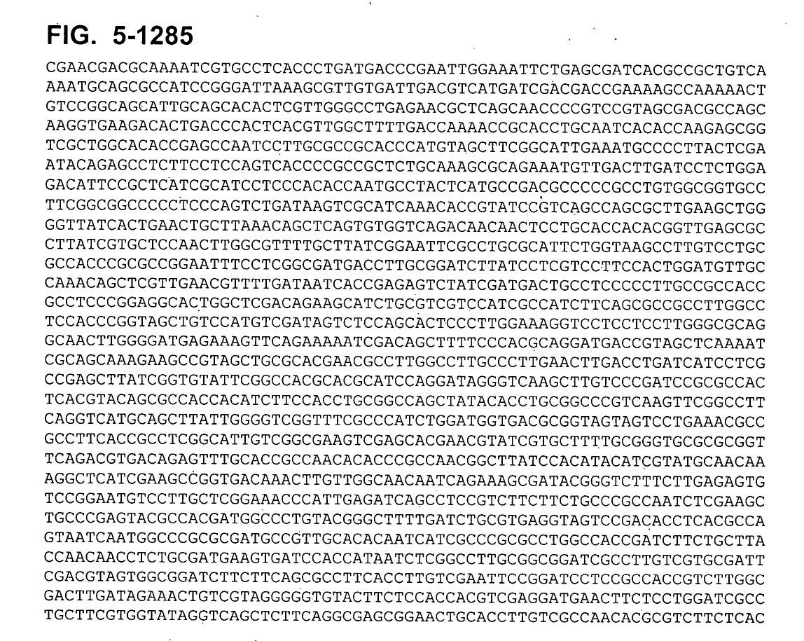

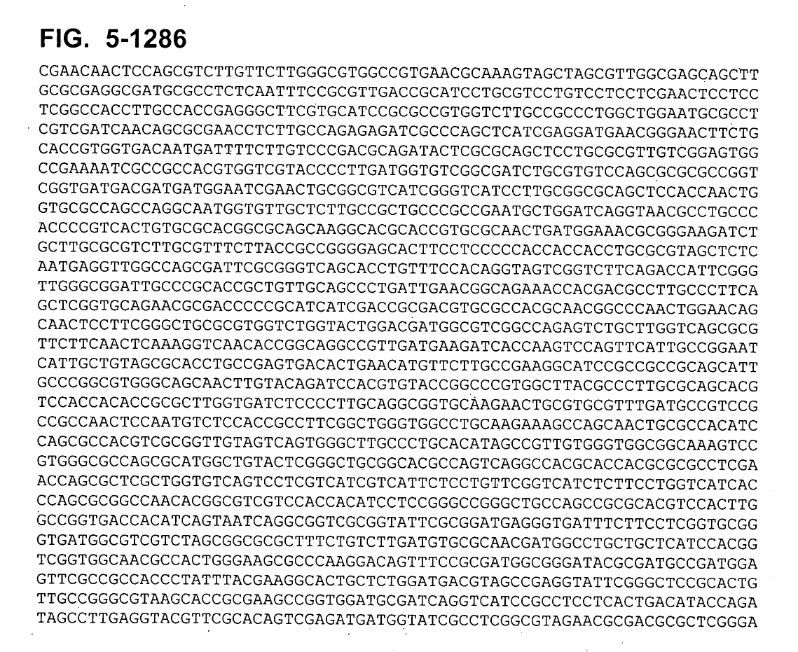

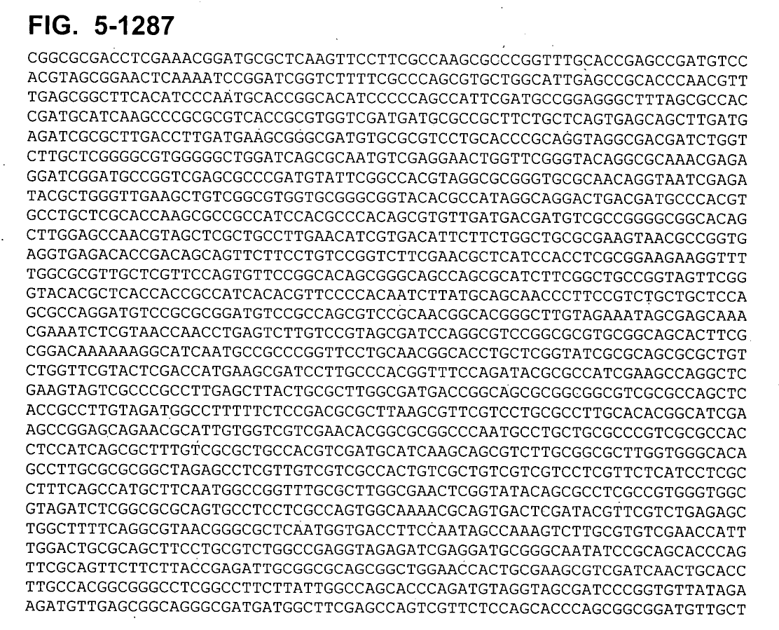

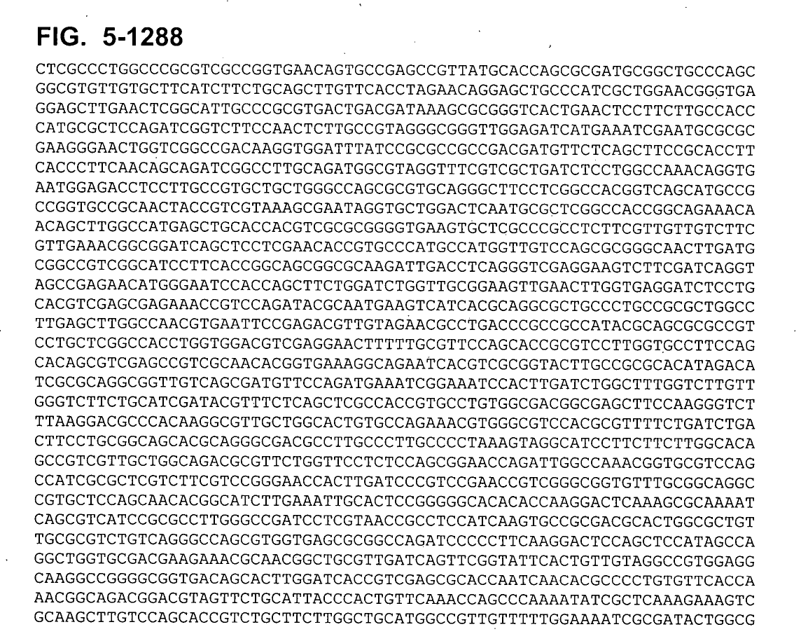

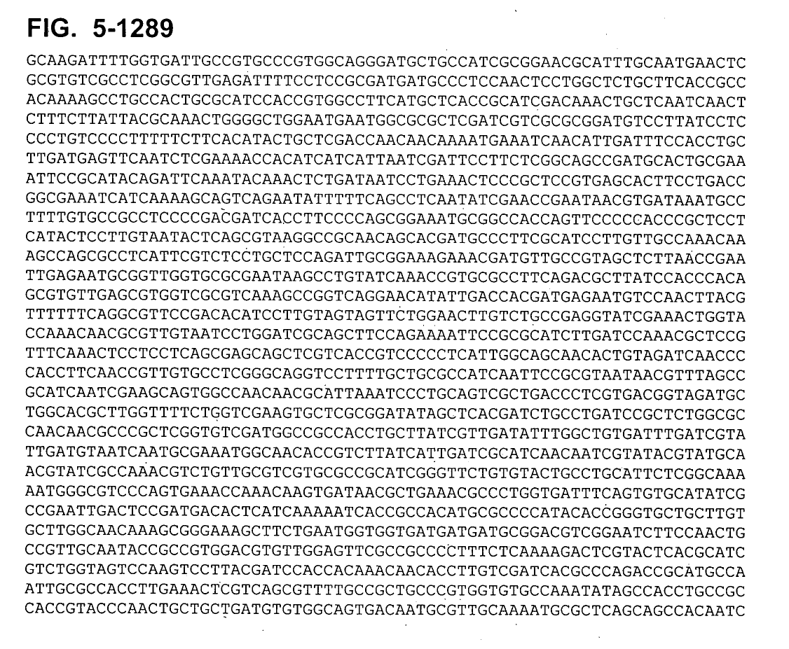

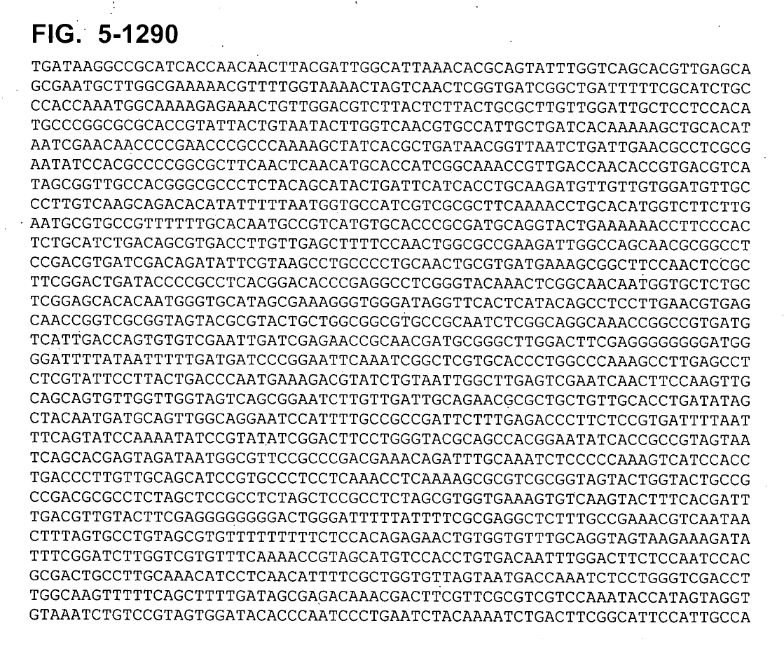

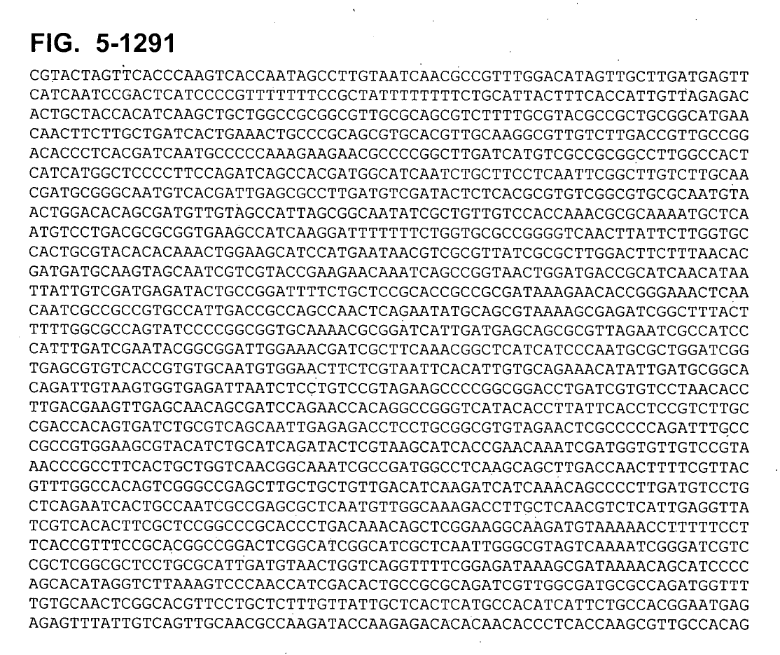

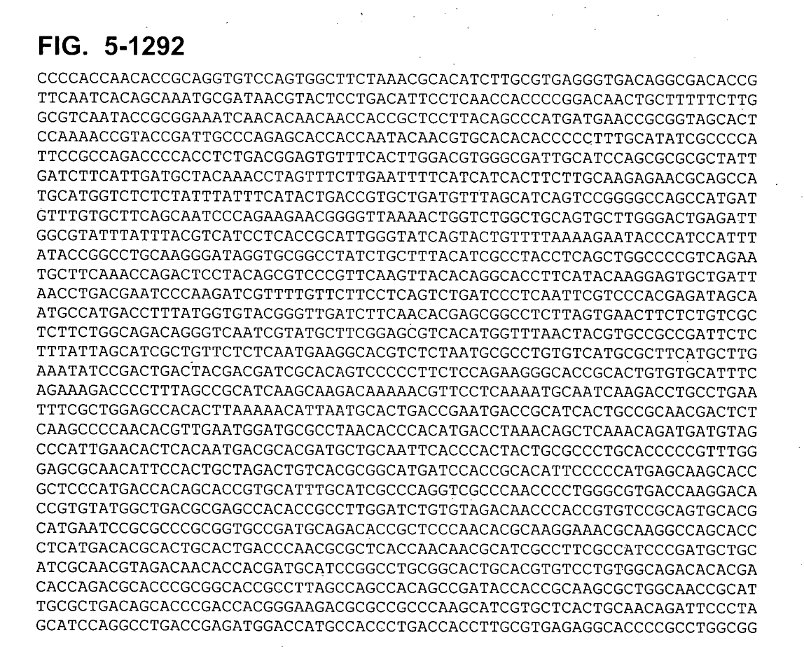

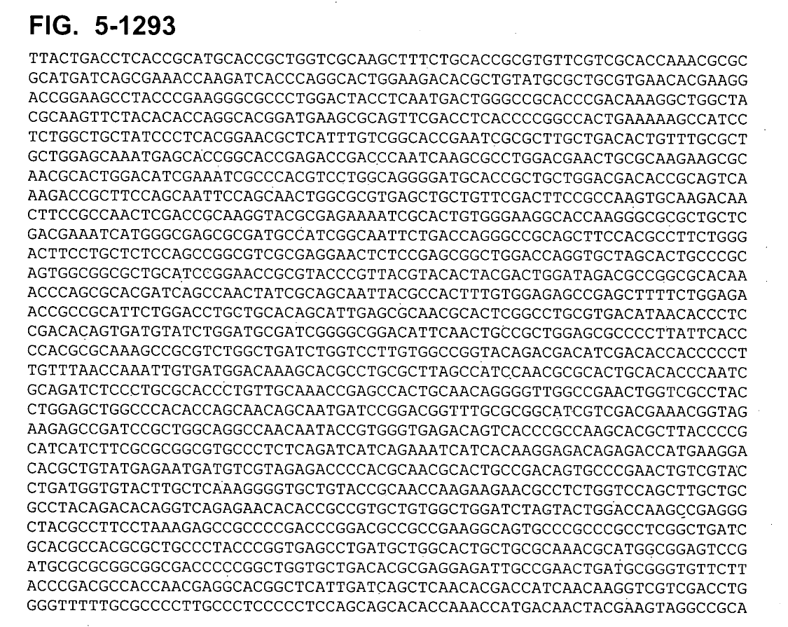

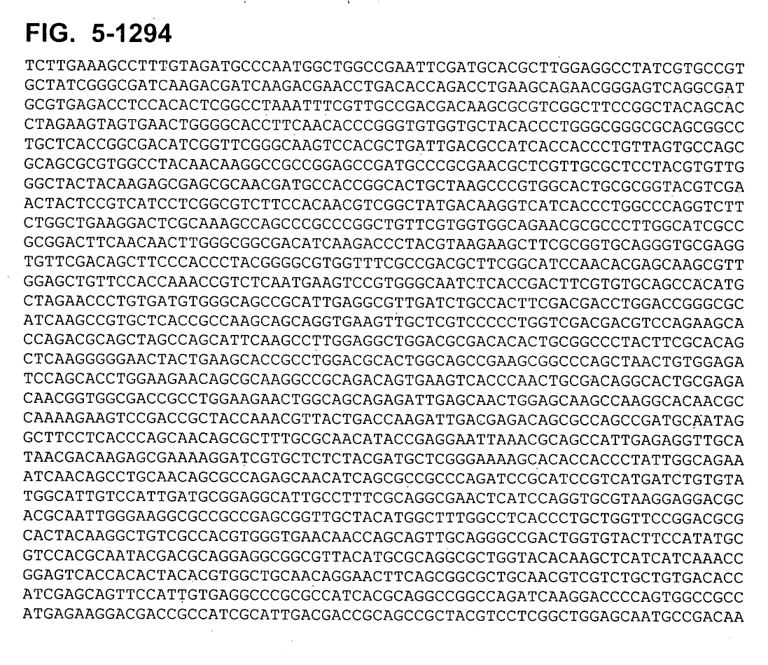

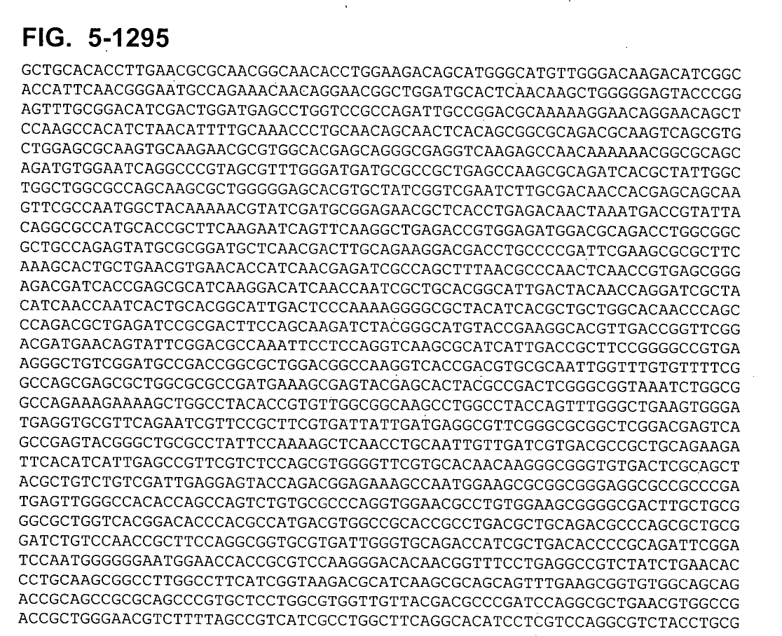

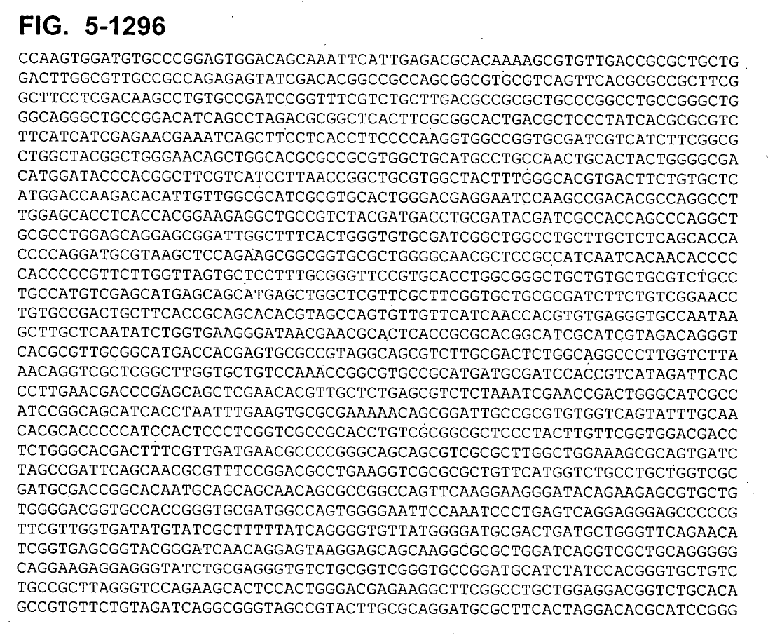

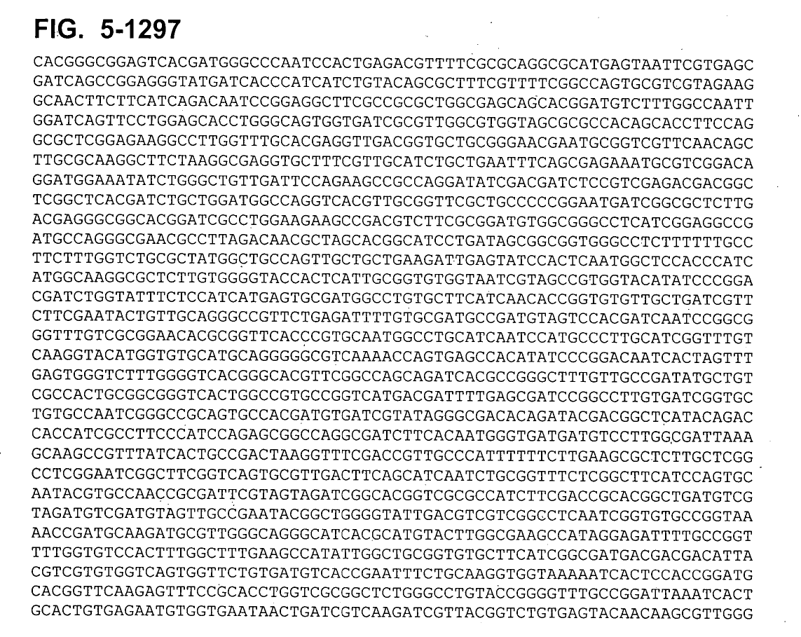

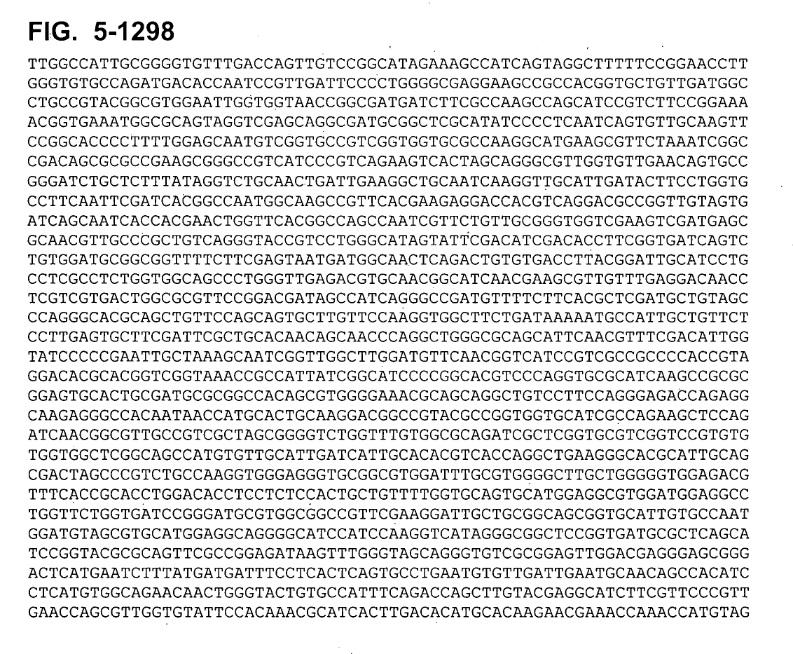

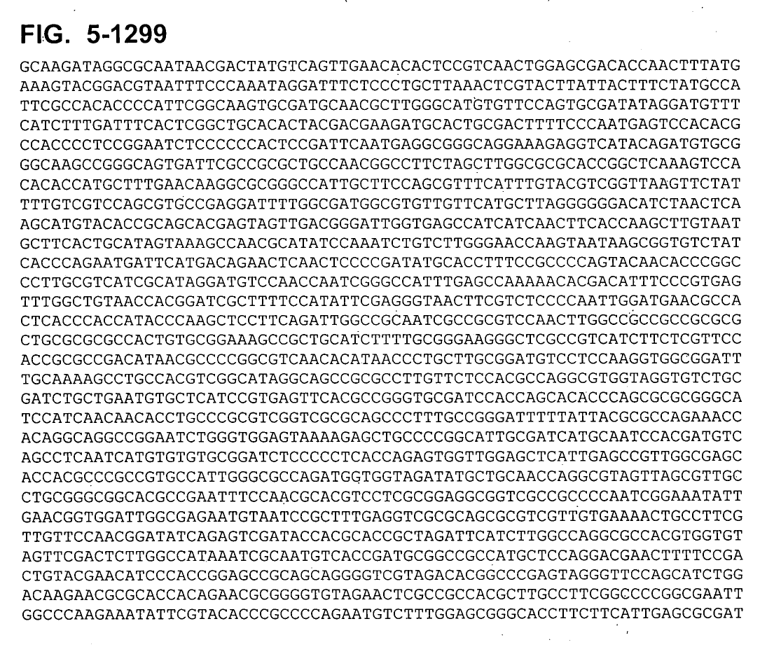

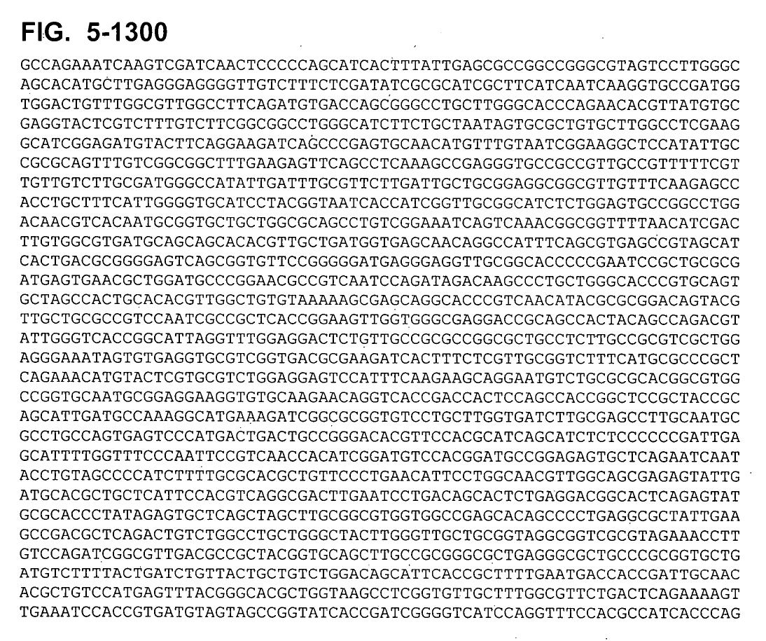

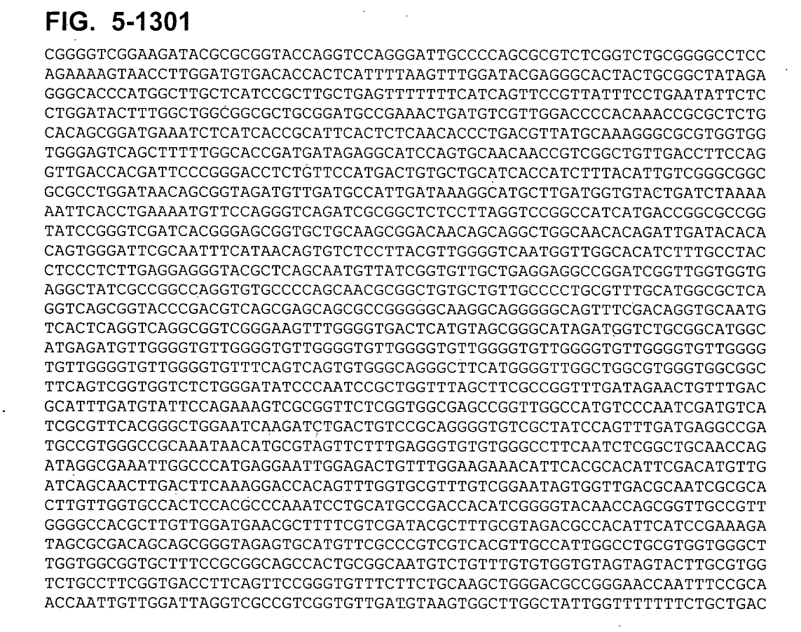

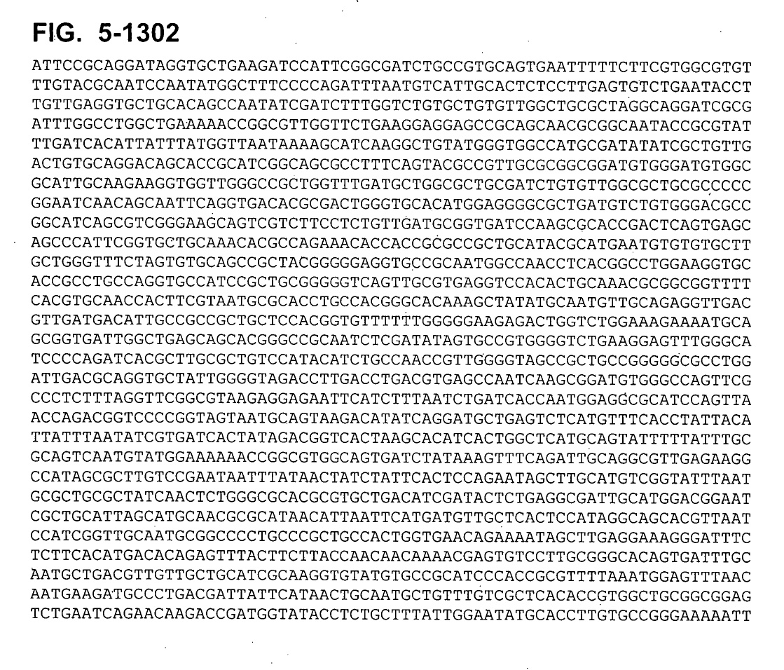

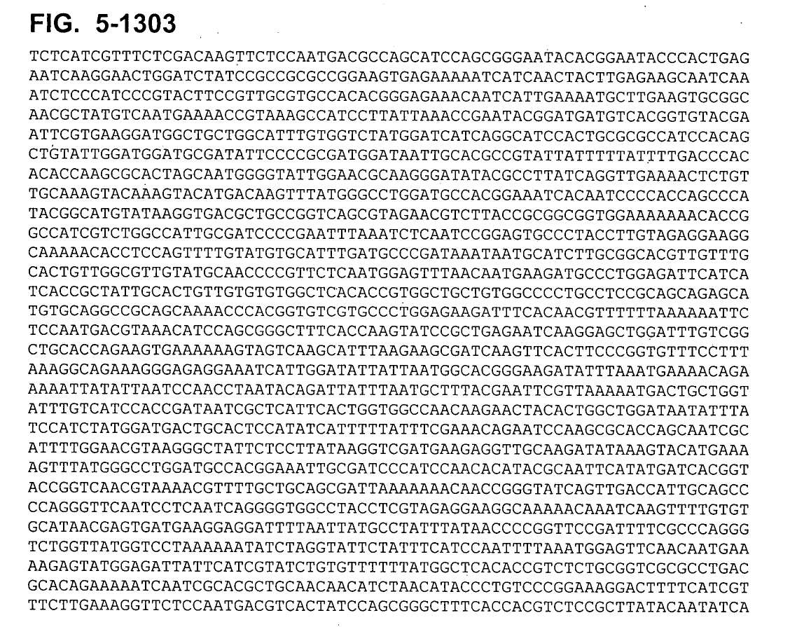

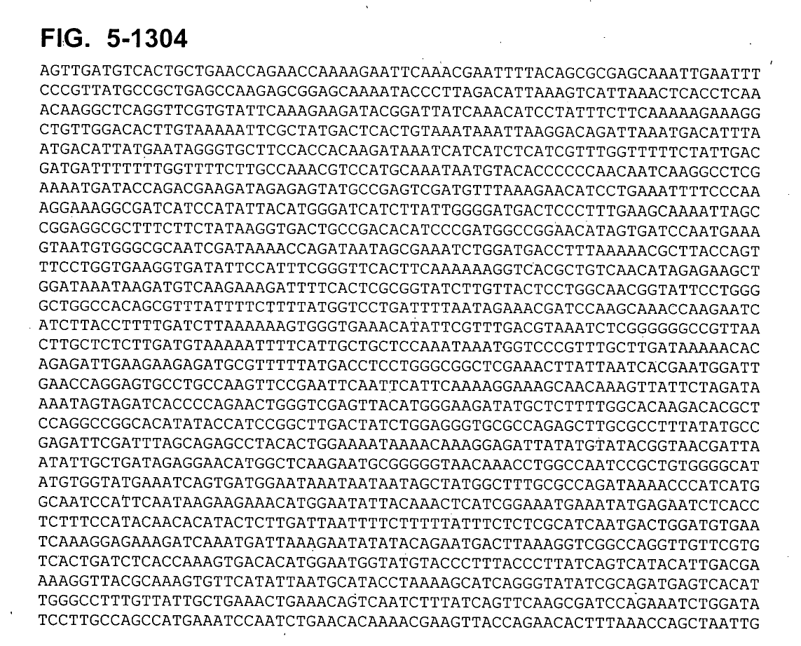

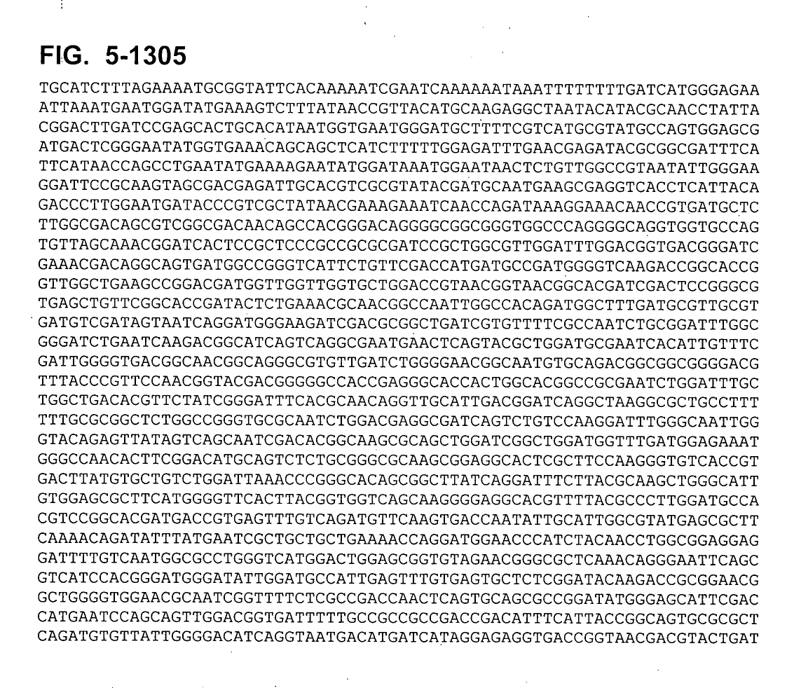

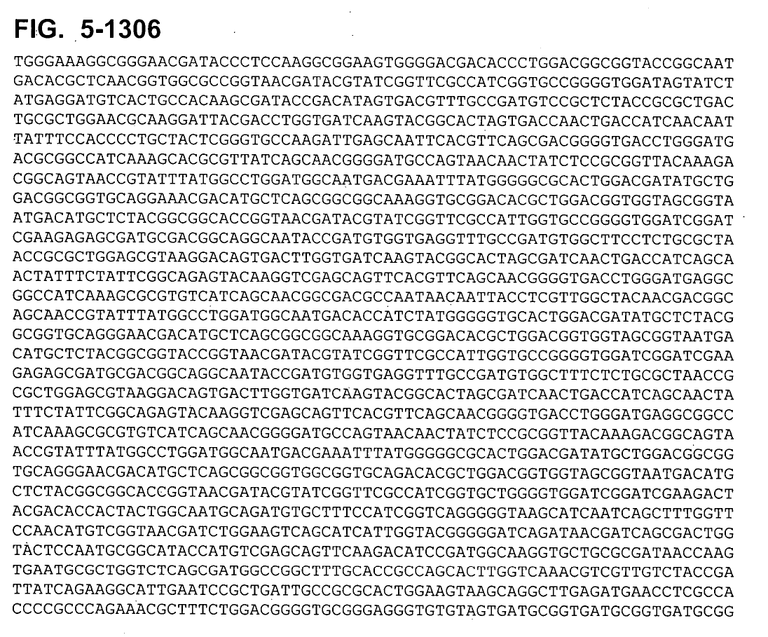

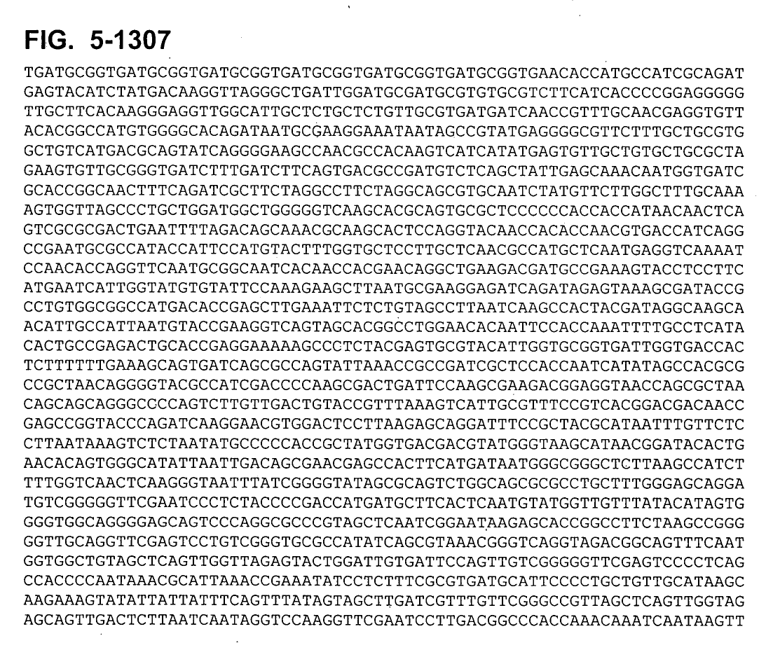

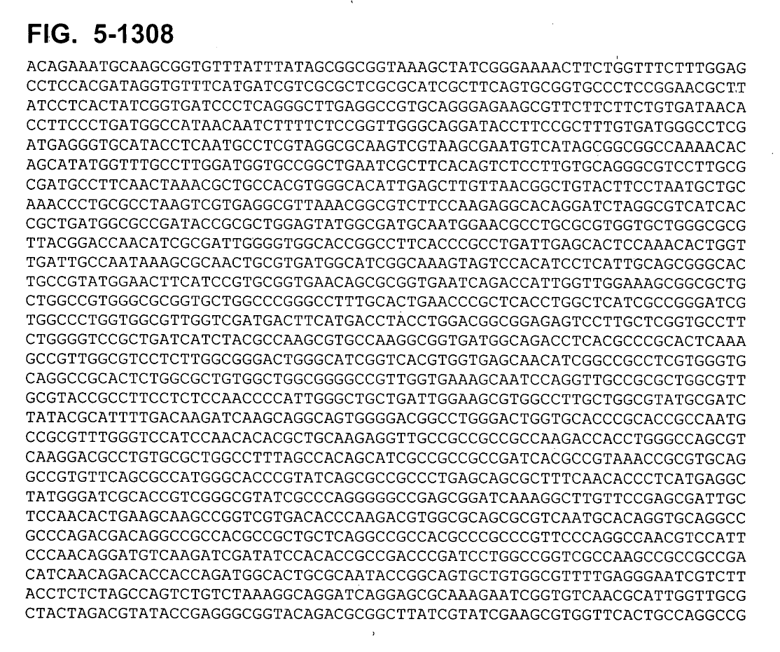

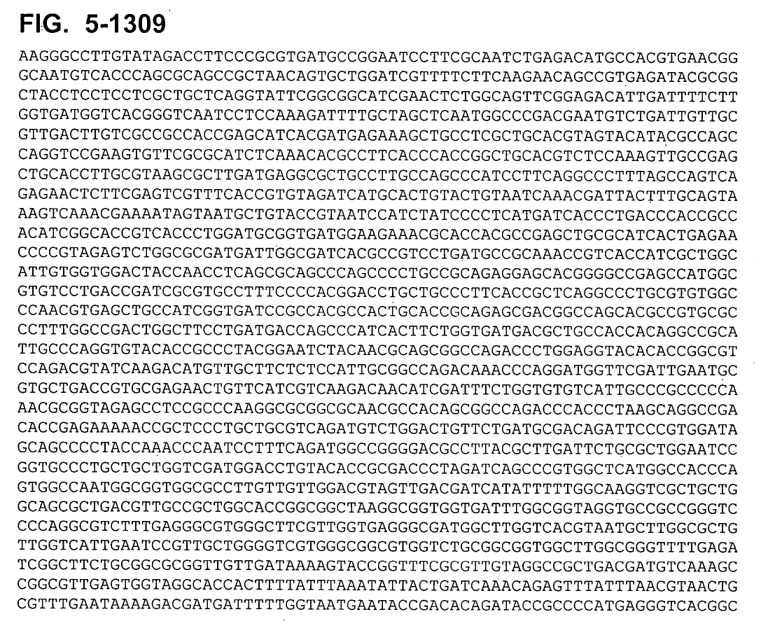

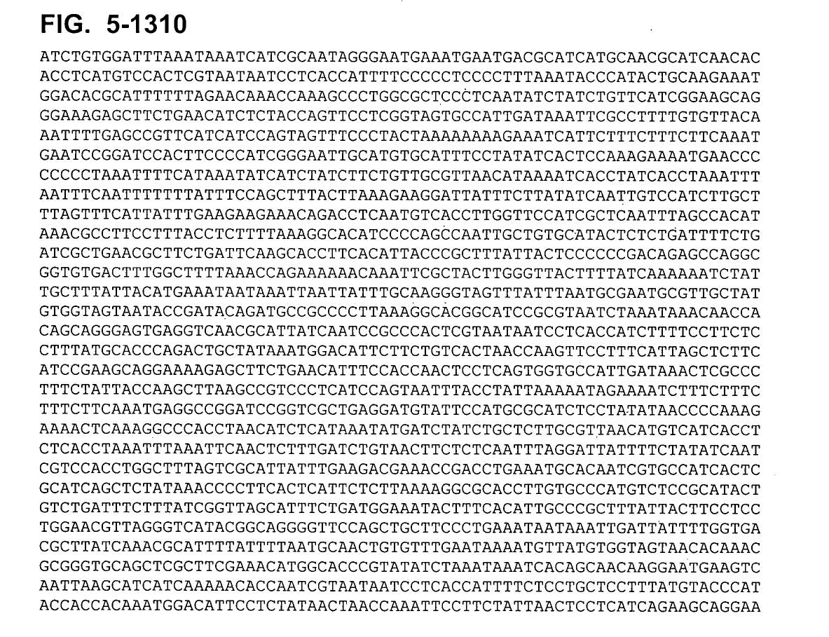

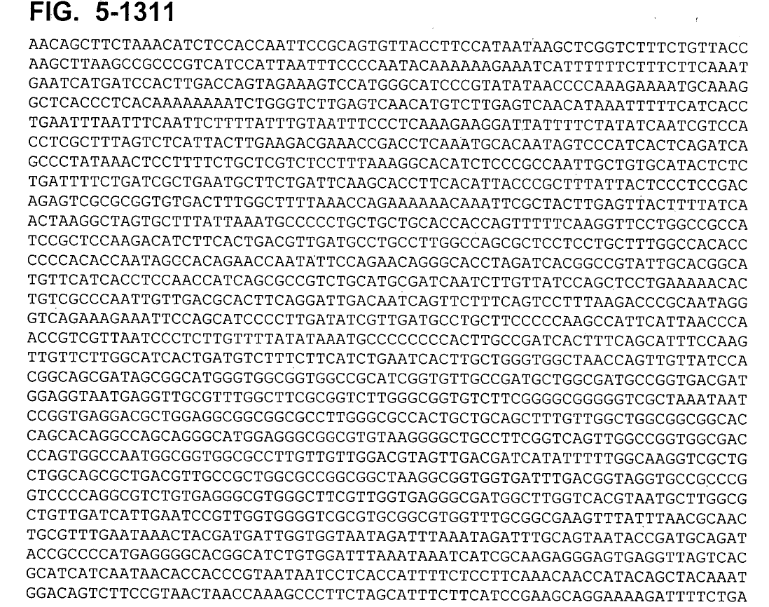

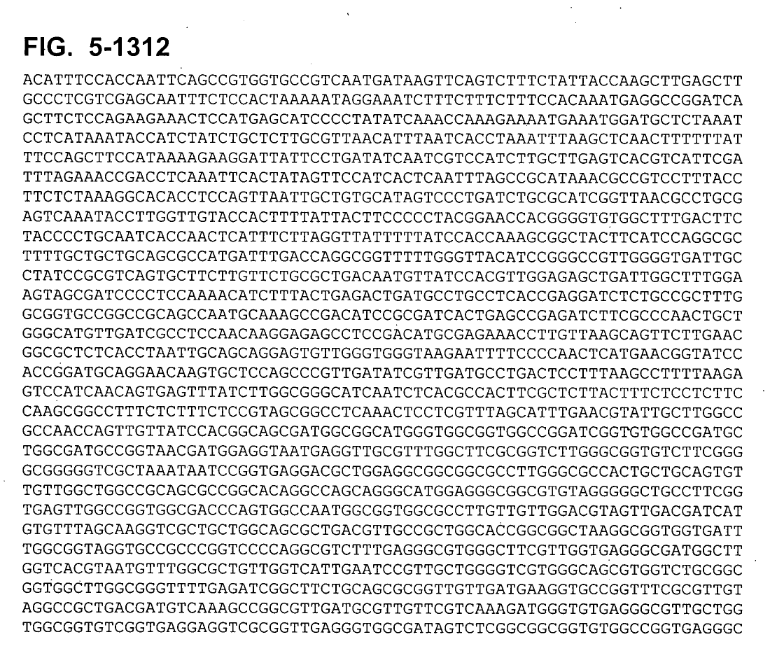

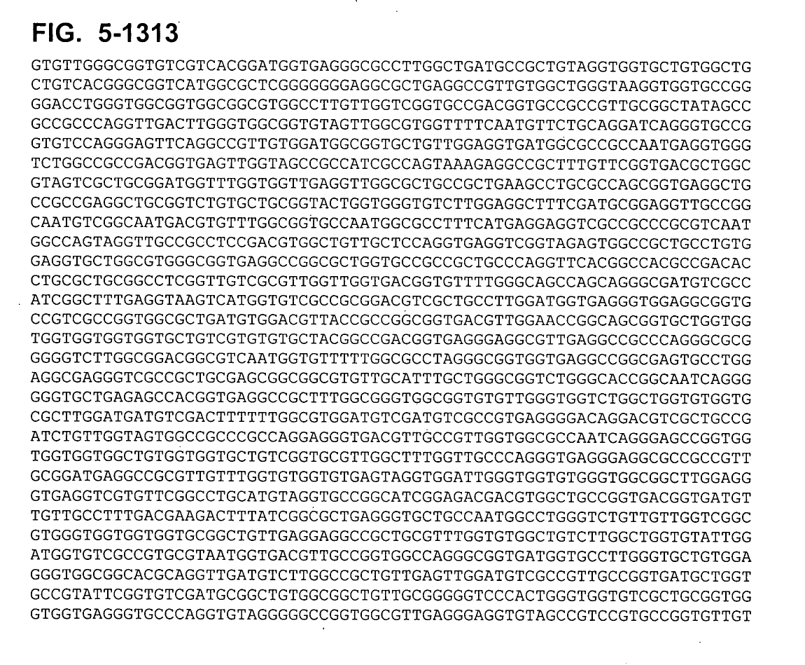

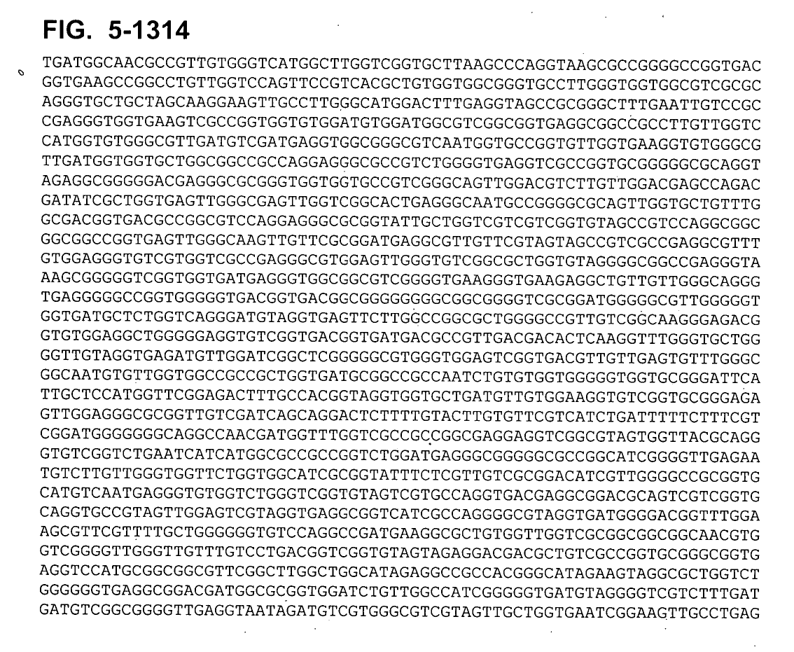

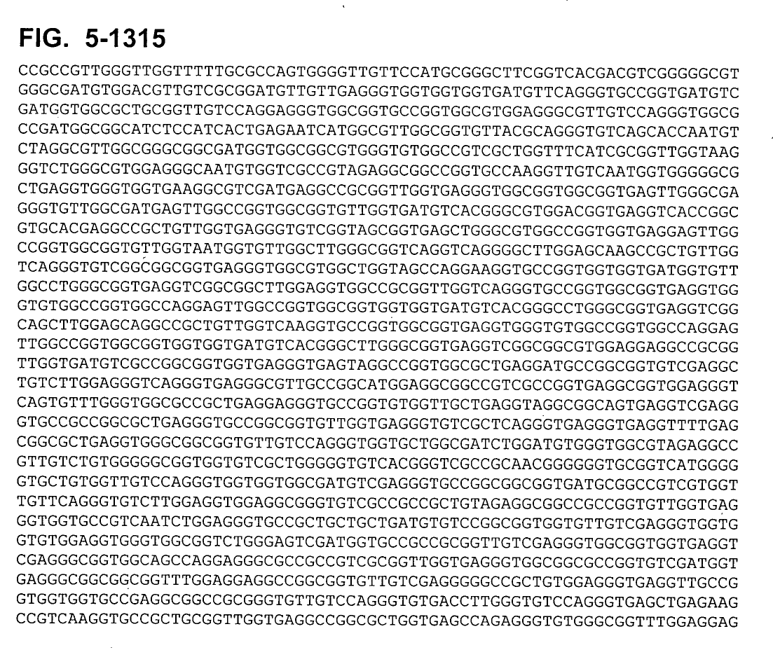

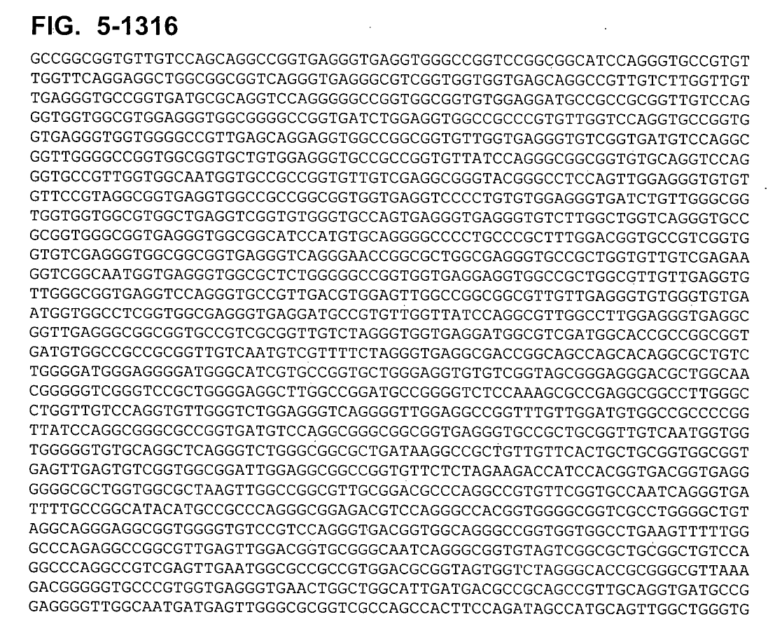

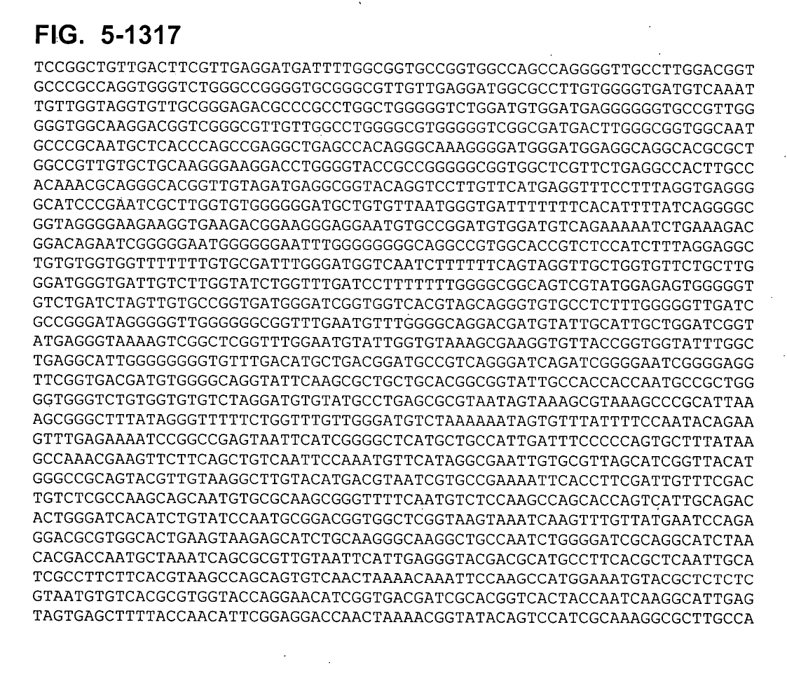

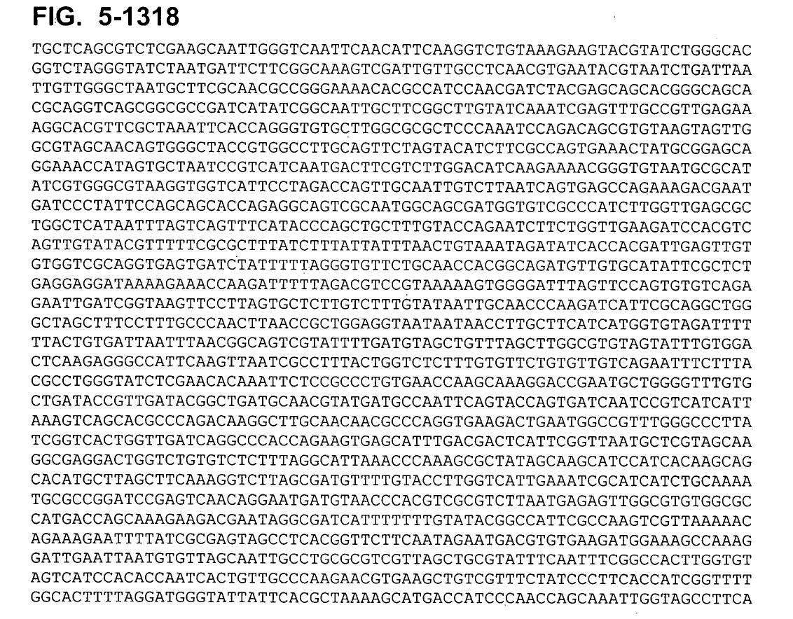

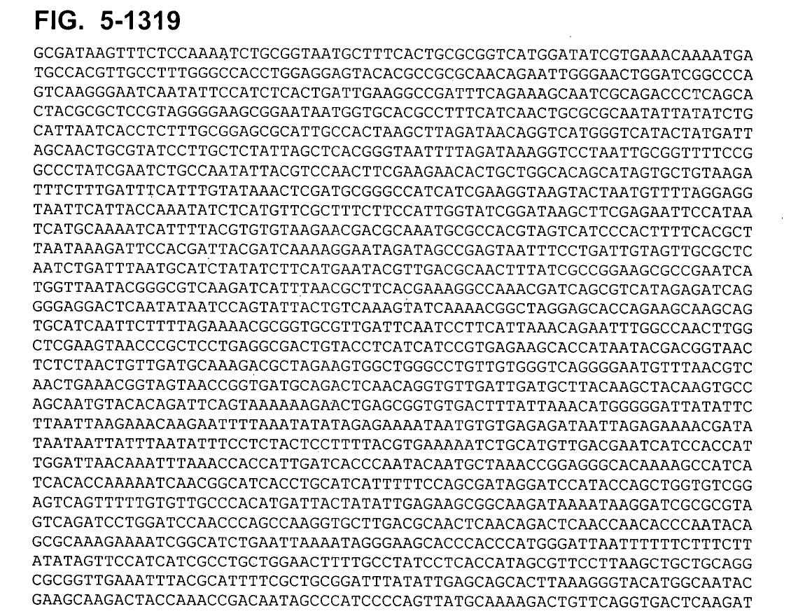

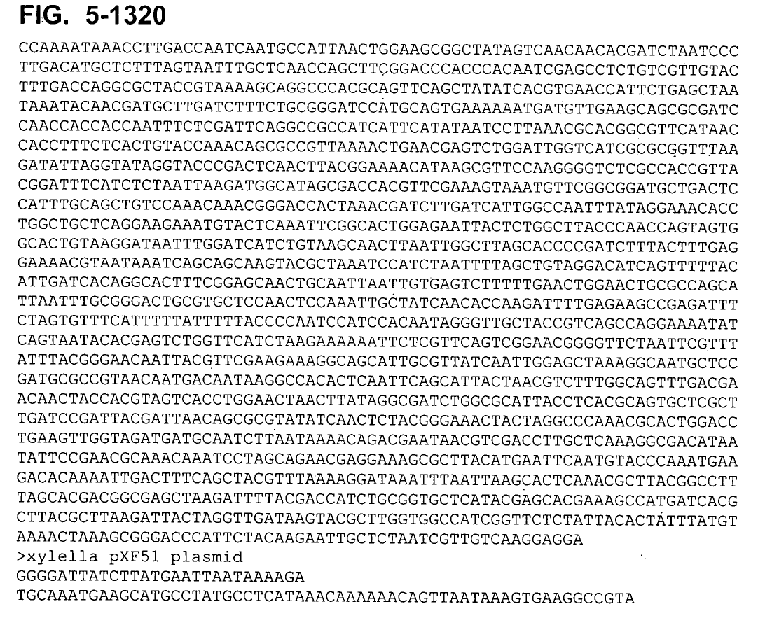

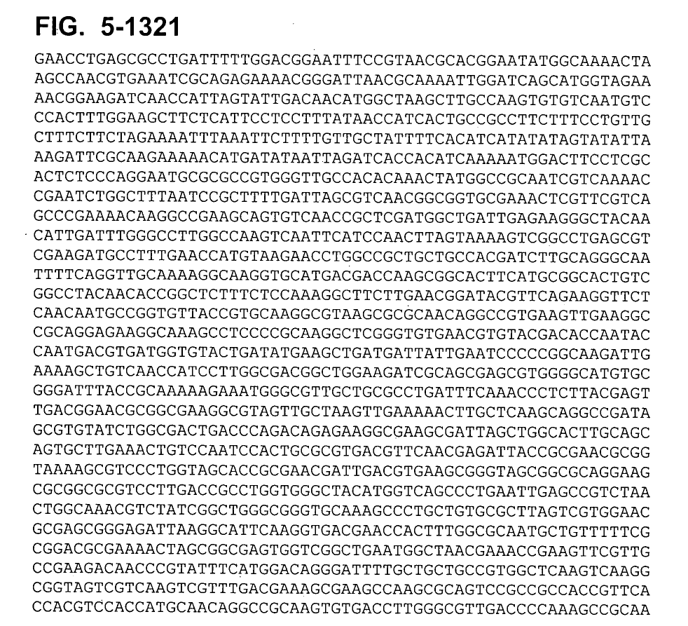

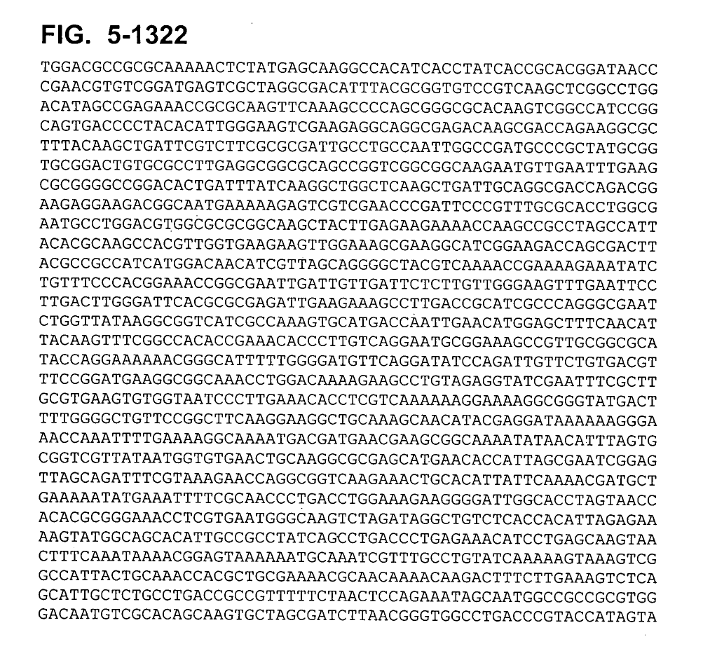

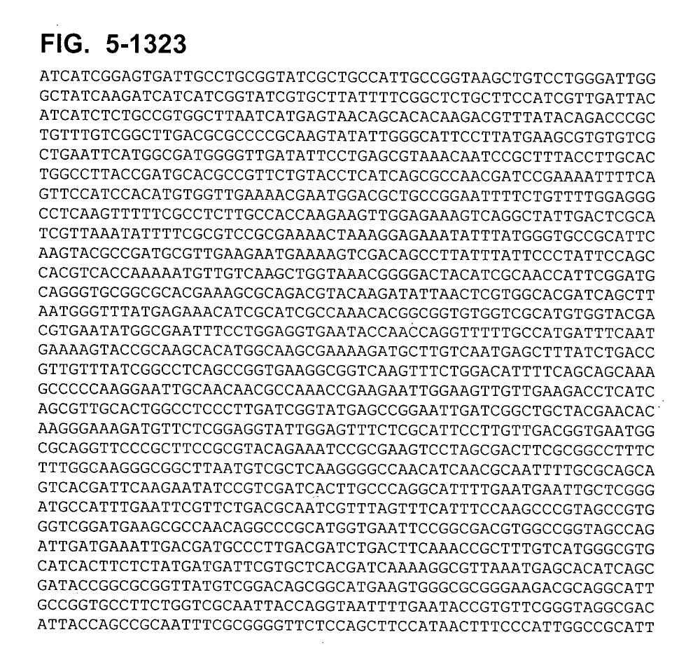

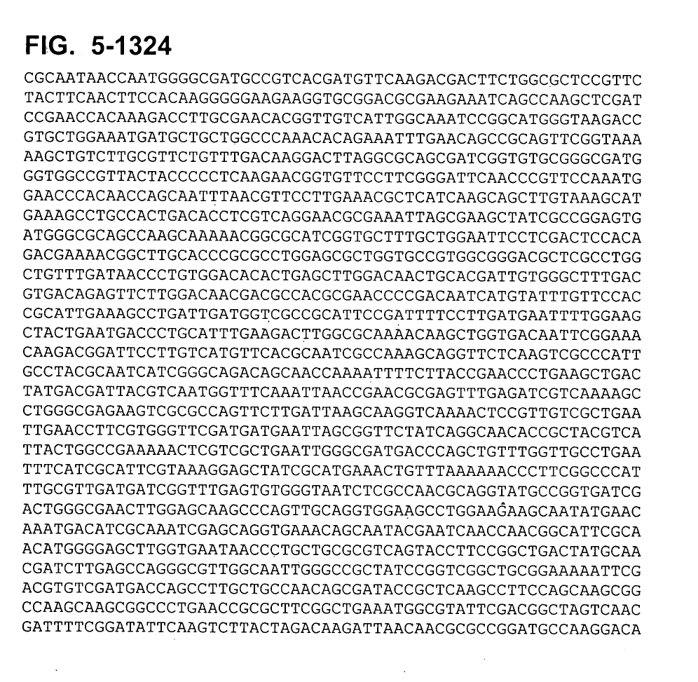

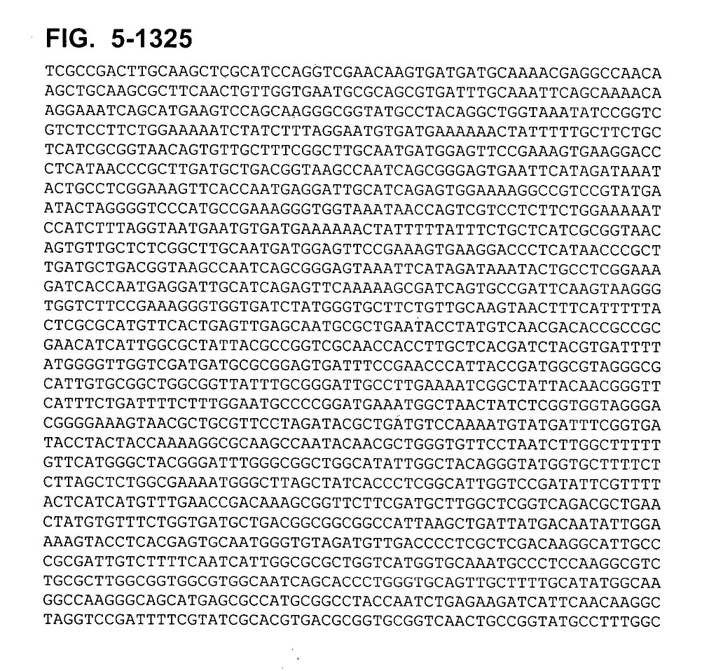

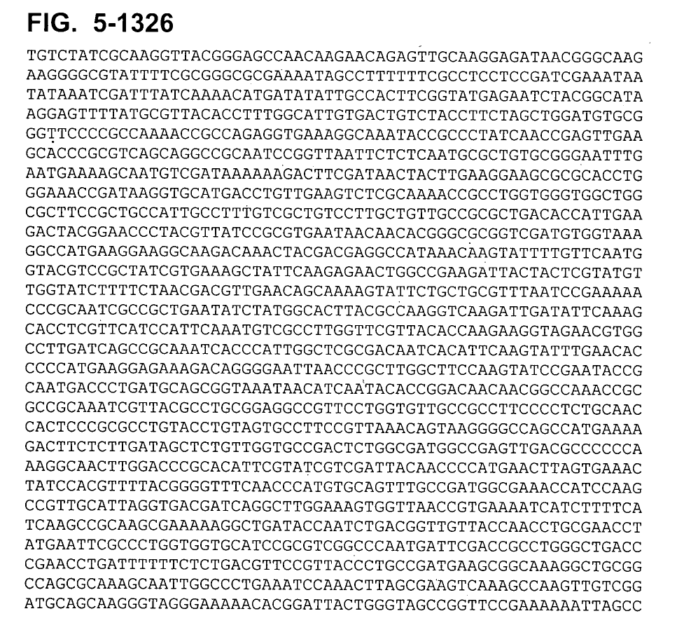

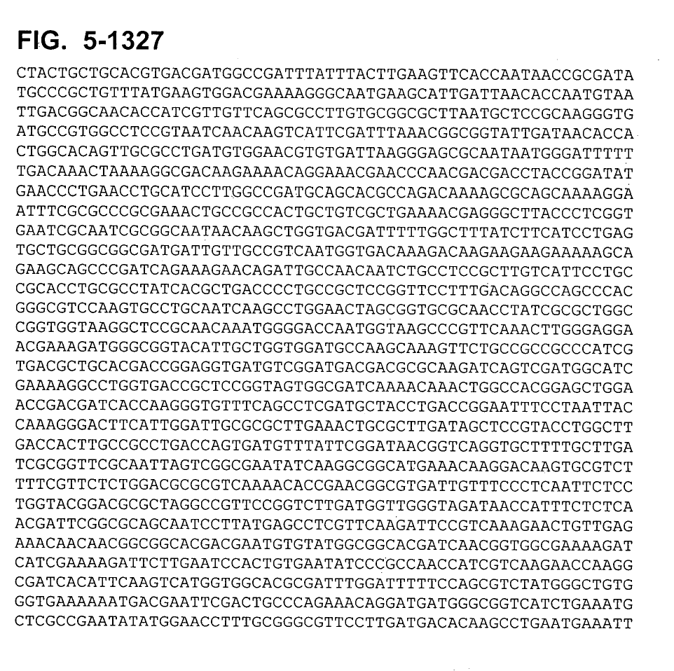

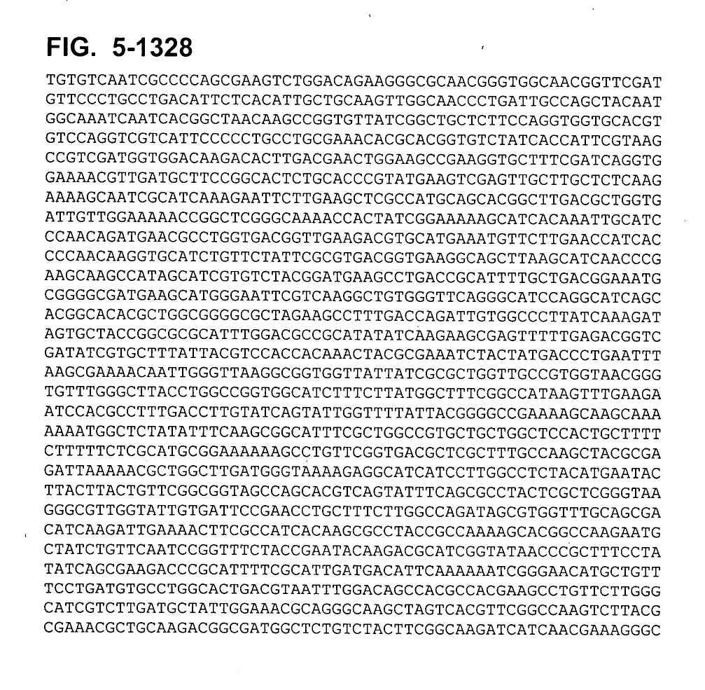

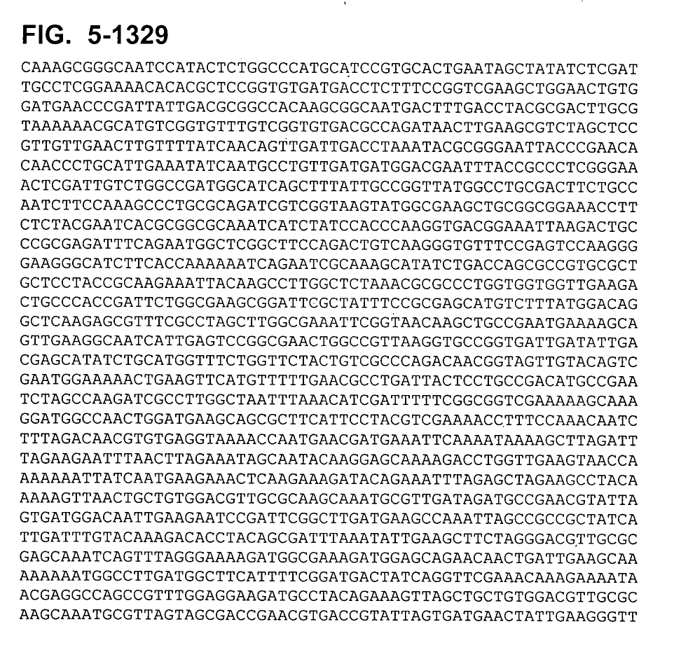

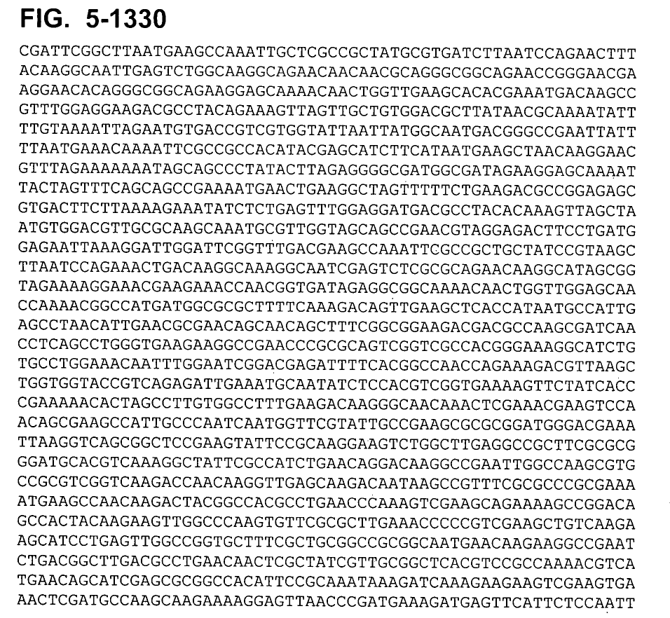

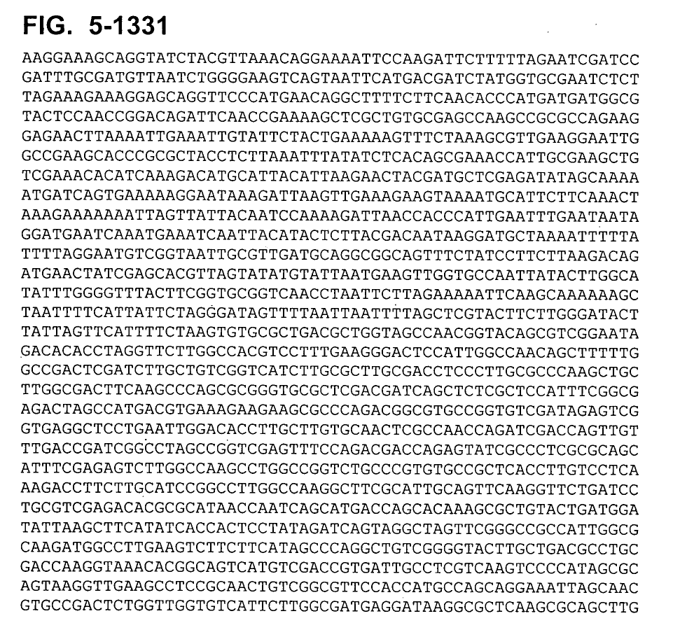

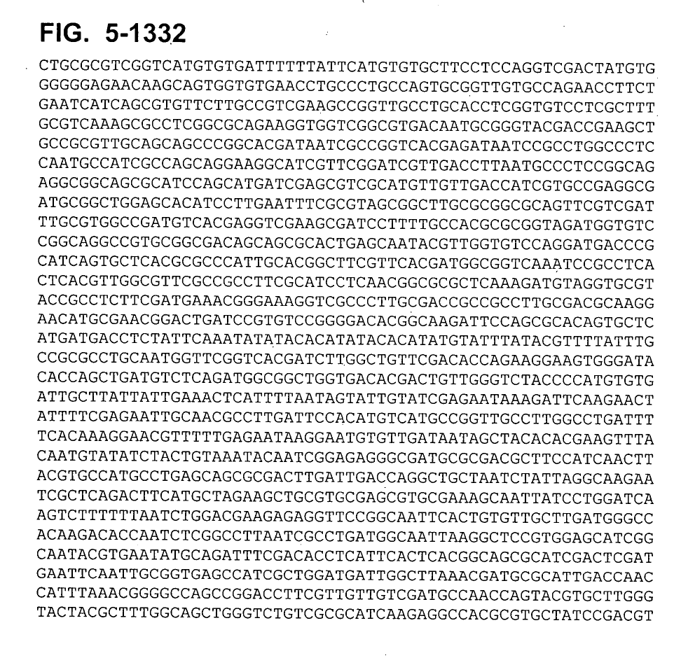

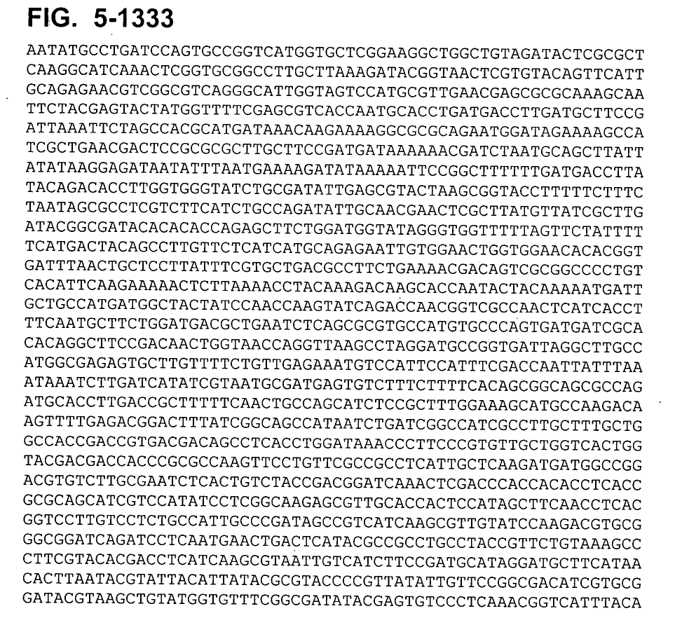

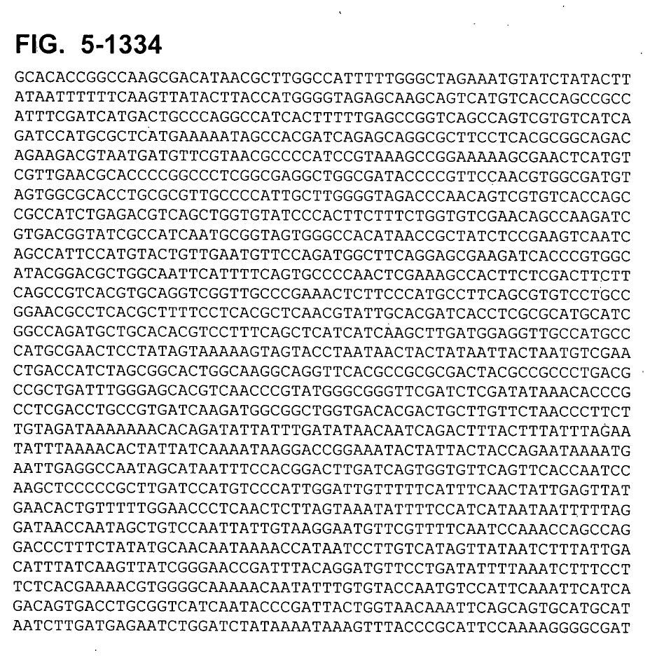

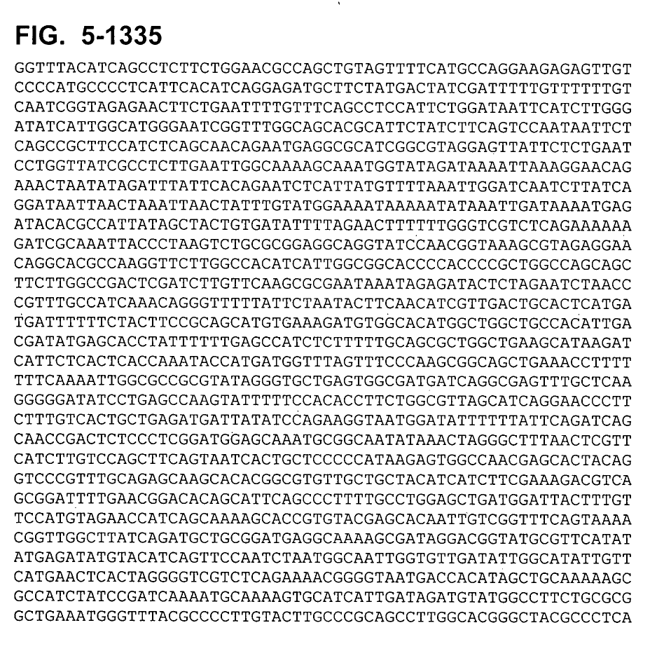

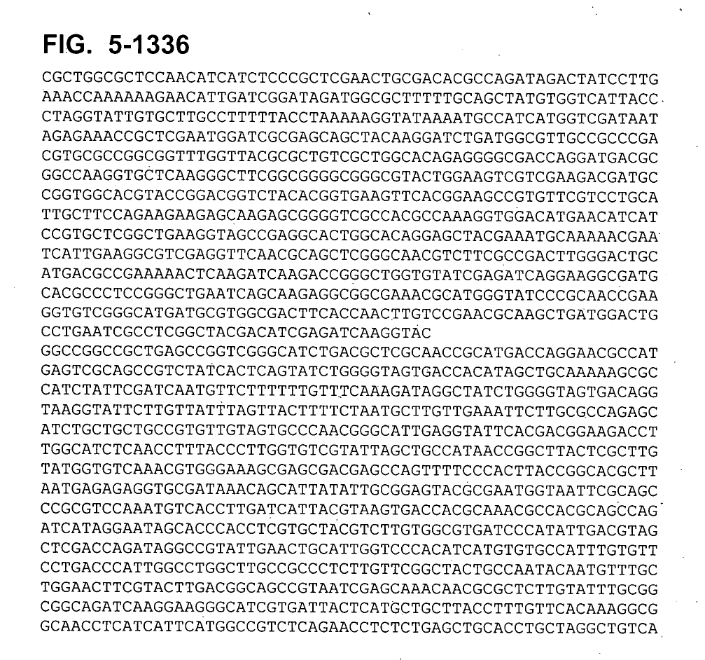

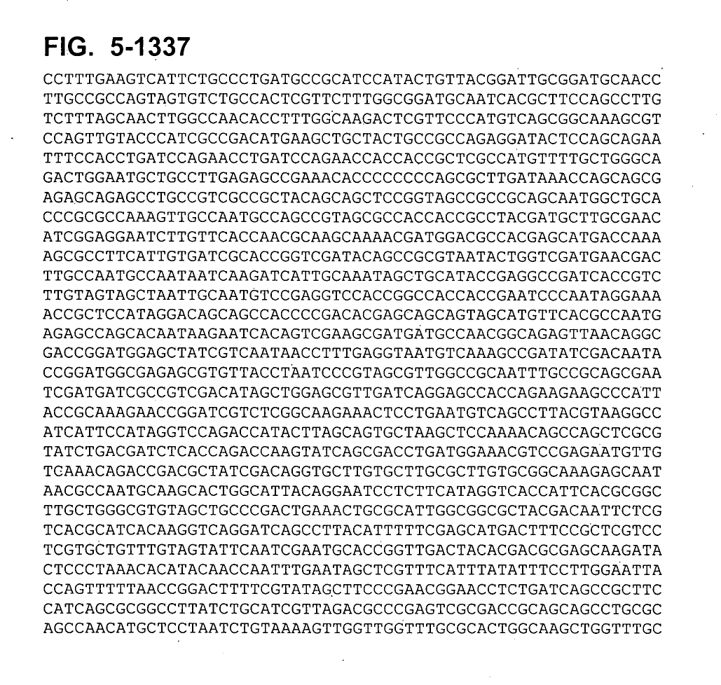

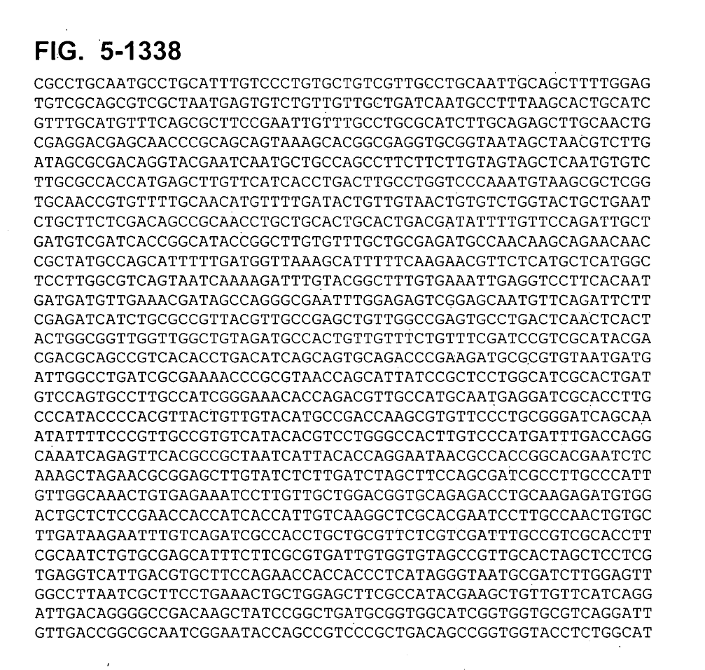

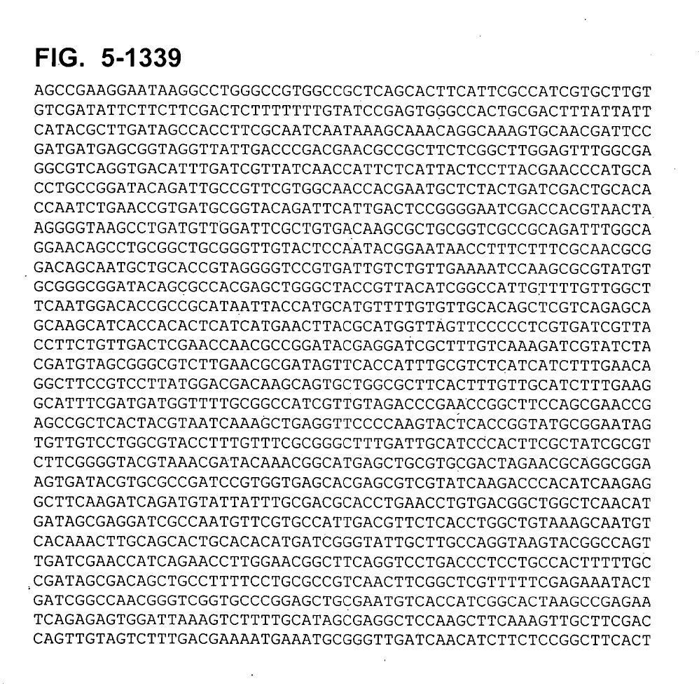

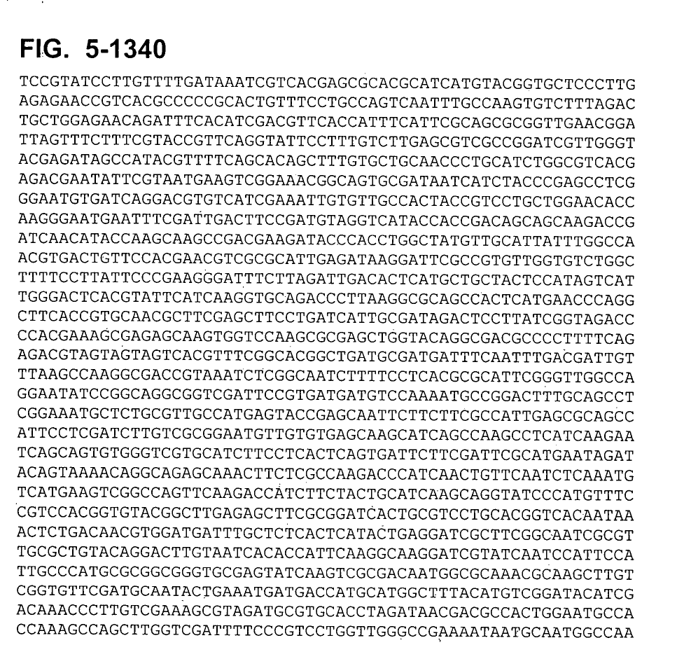

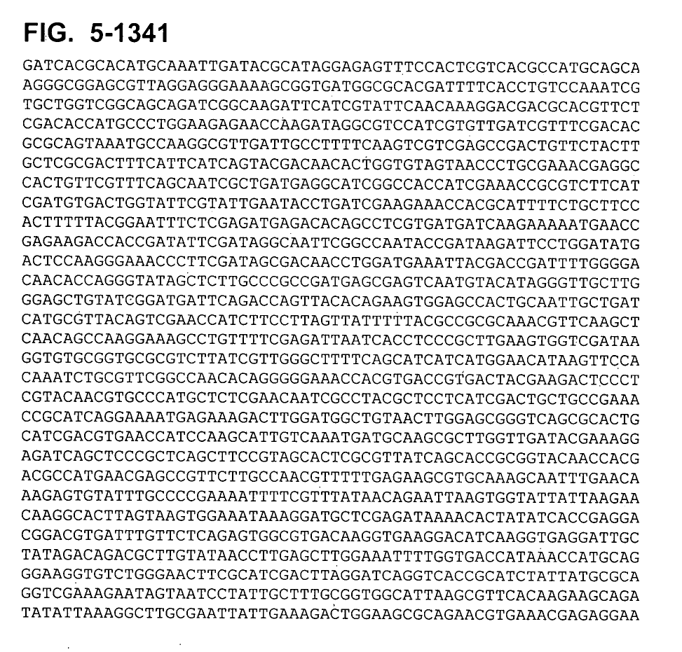

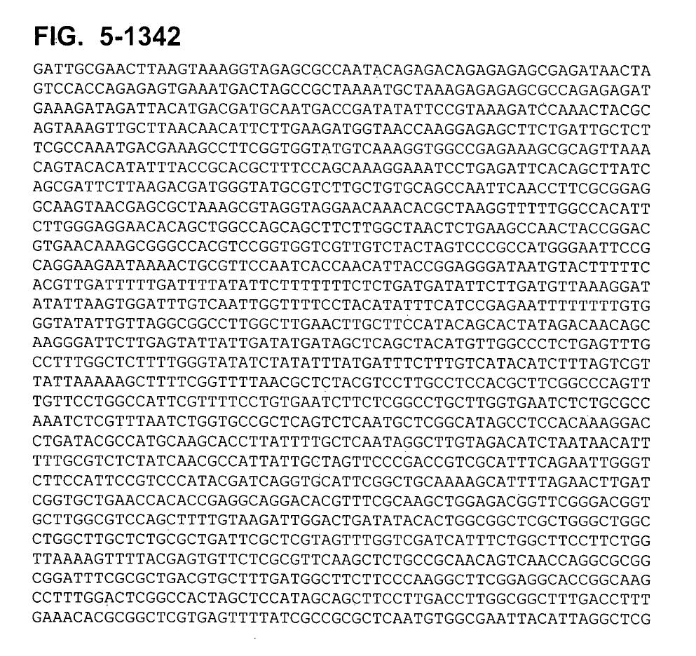

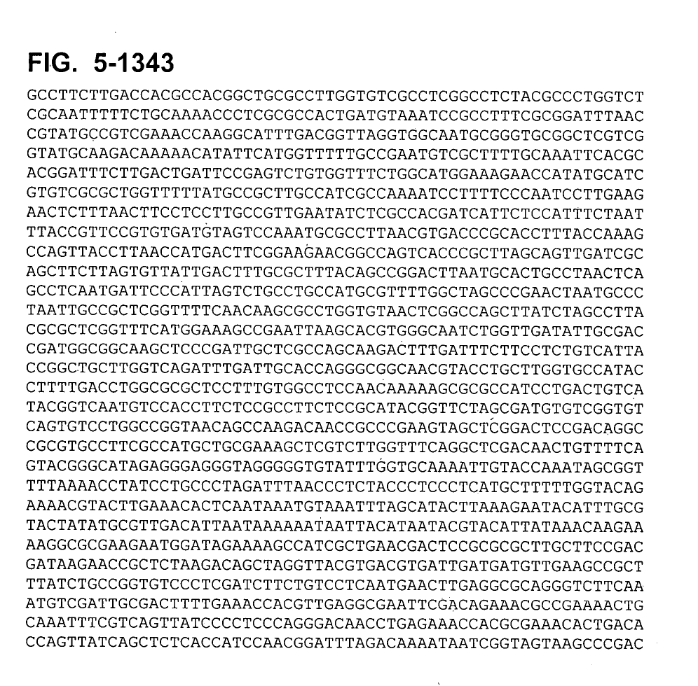

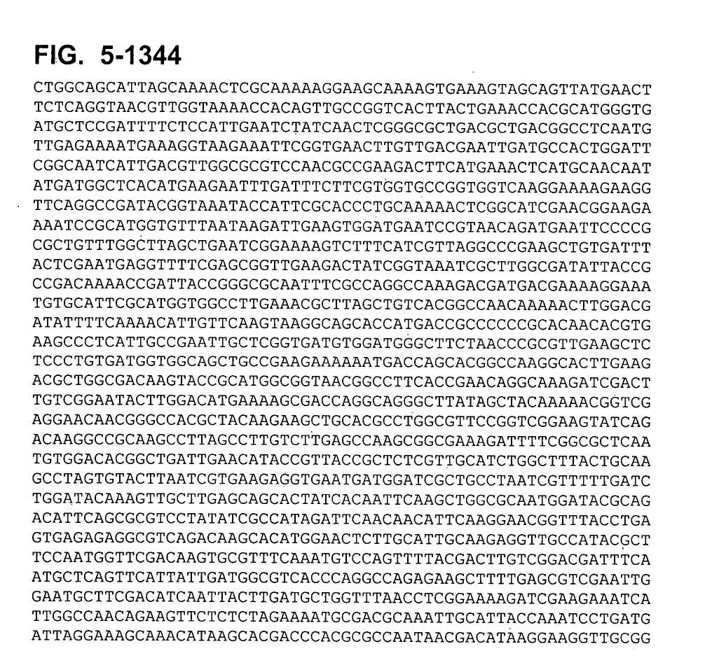

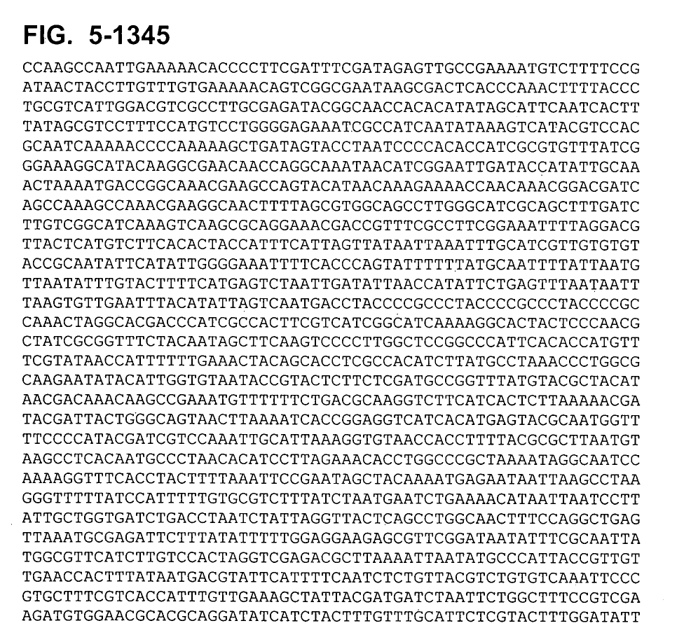

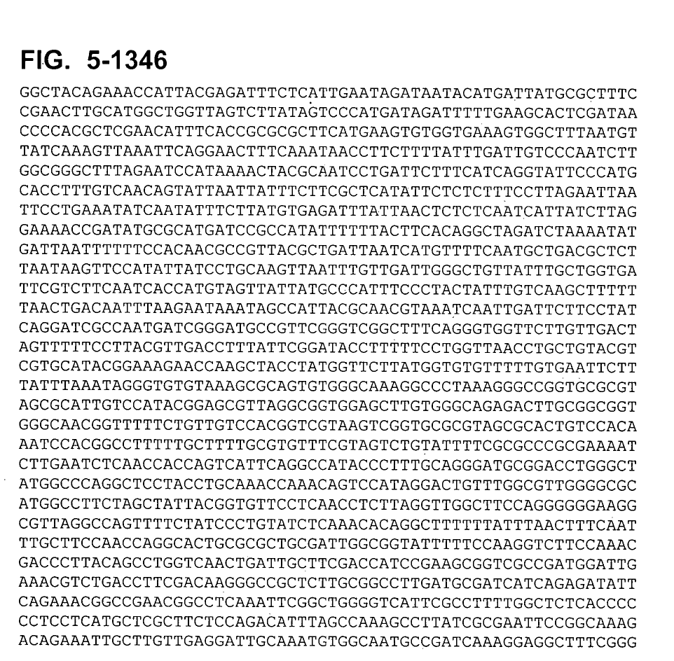

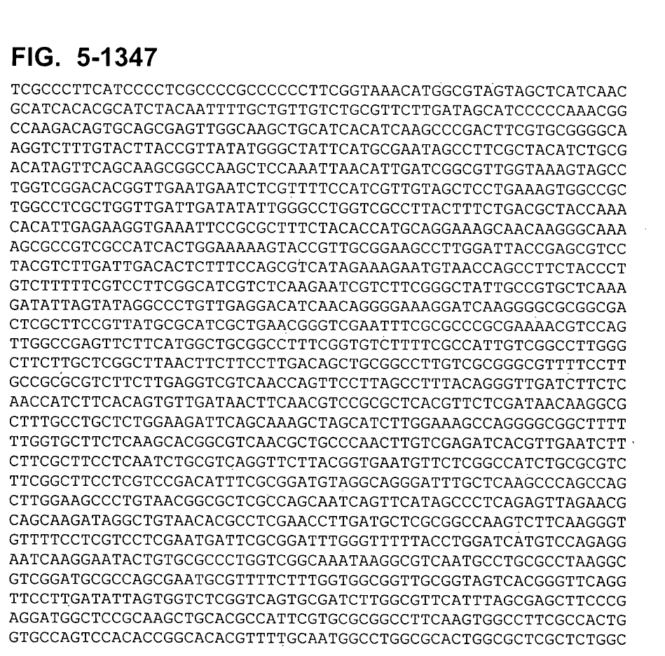

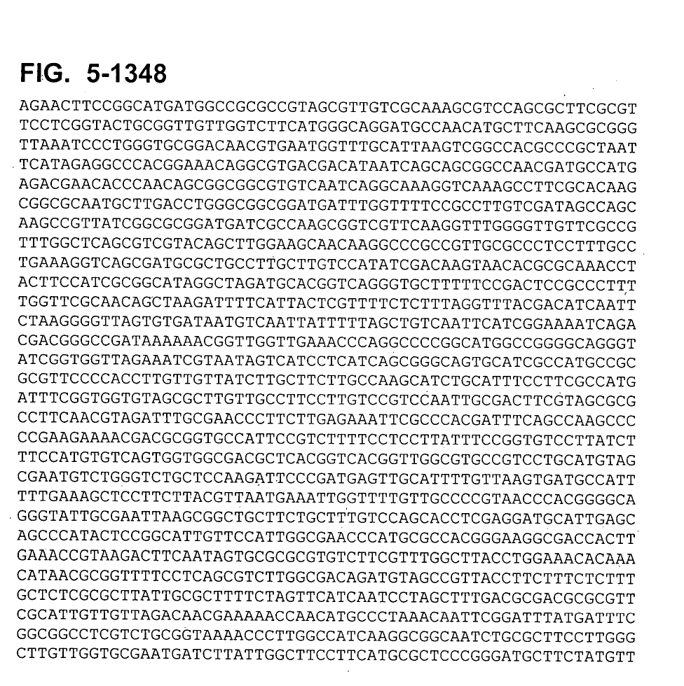

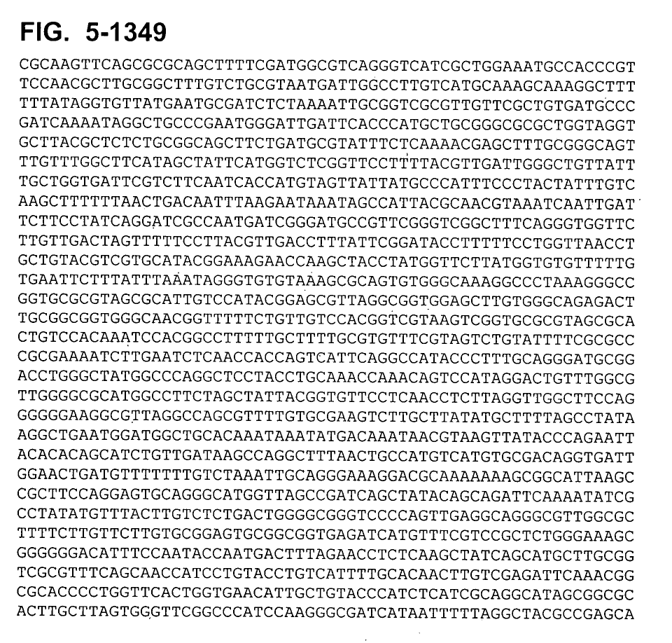

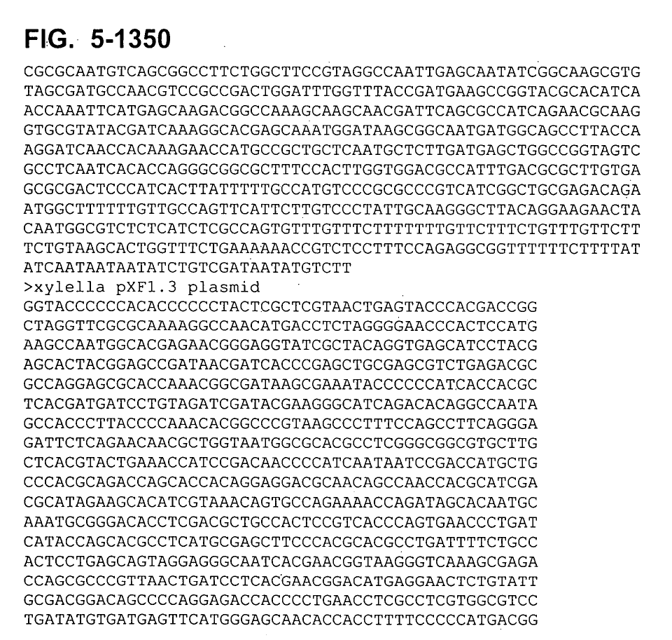

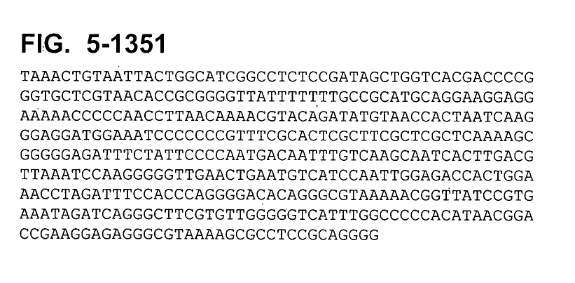

General features of the genome. The basic feature of the genome are listed in Table 1 and a detailed map is shown in FIG. 1. The conserved origin of replication of the large chromosome has been identified to a region between the putative 50S ribosomal protein L34 and gyrB genes containing dnaA, dnaN and recF. Ye et al, Curr. Microbiol 29:29-39 (1994). The Escherichia coli DnaA-box consensus sequence TTATCCACA is found on both DNA strands close to dnaA. In addition, there are typical 13-mer (ACCACCACCACCA) and 9-mer (two TTTCATTGG and two TTTTATATT) sequences in other intergenic sequences of this region. This region is coincident with the calculated GC-skew signal inversion. Francino, et al, Trends. Genet. 13:240-245 (1997). The first T of the only TTTTAT sequence in the X. fastidiosa genome found between the ribosomal protein L34 gene and dnaA has been designated base 1.

The overall percentage of ORFs for which a putative biological function could be assigned (47%) was slightly below that for other recently sequenced genomes such as Thermotoga maritima (Nelson, et al, Nature 399:323-329(1999)) (54%), Deinococcus radiodurans (White, et al, Science 286: 1571-1577 (1999)) (52.5%) or Neisseria menningitidis (Tettelin, et al, Science 287:1809-1815 (2000)) (53.7%). This may reflect the lack of previous complete genome sequences from phytopathogenic bacteria Plasmid pXF1.3 contains only two ORFs, one of which encodes a replication-associated protein. Plasmid pXF51 contains 64 ORFs, of which five encode proteins involved in replication or plasmid stability and 20 encode proteins potentially involved in conjugative transfer. One ORF encodes a protein similar to the virulence-associated protein D (VapD), found in many other bacterial pathogens. (Katz, et al, Infect. Immun 60:4586-4592 (1992)). Four regions of pXF51 present significant DNA similarity to parts of transposons found in plasmids from other bacteria, suggesting interspecific horizontal exchange of genetic material.

The principal paralogous families are summarized in Table 2. The complete list of ORFs with assigned function is shown in Table 3. Seventy-five proteins present in the 21 completely sequenced genomes in the COG database (Tatusov, et al, Nucleic Acids Res 28:33-36 (2000)) as of Mar. 15th, 2000, were also found in X.fastidiosa. Each of these sequences was used to generate a phylogenetic tree of the 22 organisms. In 69% of such trees X.fastidiosa was grouped with Haemophilus influenzae and Escherichia coli, consistent with a phylogenetic analysis undertaken with the 16S rRNA gene (Preston, et al, Curr. Opin. Microbiol 1:589-597 (1998)).

One ORF, a cytosine methyltransferase (XF1774), is interrupted by a Group II intron. The intron was identified on the basis of the presence of a reverse transcriptase-like gene (as in other Group II introns), conserved splice sites, conserved sequence in structure V and conserved elements of secondary structure (Knoop, et al, J. Mol. Biol 242:389-396 (1994)). Group II introns are rare in prokaryotes, but have been found in different evolutive lineages including E. coli, cyanobacteria and proteobacteria (Ferat, et al, Nature 364:358-361 (1993)).

Transcription, translation and repair. The basic transcriptional and translational machinery of X. fastidiosa is similar to that of E. coli (Blattner, et al, Science 277:1453-1474 (1997)). Recombinational repair, nucleotide and base excision repair and transcription-coupled repair are present with some noteworthy features. For example, no photolyase was found, implicating exclusively dark repair. Although the main genes of the SOS pathway, recA and lexA, are present, ORFs corresponding to the three DNA polymerases induced by SOS in E. coli (DNA polymerases II, IV and V) (Bridges, Curr. Biol 9:R475-R477 (1999)) are missing, indicating that the mutational pathway itself may be distinct.

Energy metabolism. Even though X. fastidiosa is, as its name suggests, a fastidious organism, energy production is apparently efficient. In addition to genes for all the components of the glycolytic pathway, all genes for the tricarboxylic acid cycle as well as the oxidative and electron transport chains are present. ATP synthesis is driven by the resulting chemiosmotic proton gradient and occurs via an F-type ATP synthase. Fructose, mannose and glycerol can be utilised in addition to glucose in the glycolytic pathway. A complete pathway for hydrolysis of cellulose to glucose, consisting of 1,4-β-glucanase, and β-glucanase, and β-glucosidase, is present, suggesting that cellulose breakdown may supplement the often low concentrations of monosaccharides in the xylem (Brodbeck, et al, Arch. Insect Biochem. Physiol 42:37-50 (1999)). Two lipases are encoded in the genome, but there is no β-oxidation pathway for the hydrolysis of fatty acids, presumably precluding their utilisation as an alternative carbon and energy source. Likewise, although enzymes required for the breakdown of Thr, Ser, Gly, Ala, Asp and Glu are present, pathways for the catabolism of the other naturally occurring amino acids are incomplete or absent.

The gluceonogenesis pathway appears to be incomplete. Phosphoenolpyravate carboxykinase and the gluconeogenic enzyme fructose-1,6-bisphosphatase, required to bypass the irreversible step in glycolysis, are not present. The absence of the first is compensated by the presence of phosphoenolpyruvate synthase and malate oxidoreductase, which together can generate phosphoenolpyruvate from malate. There appears, however, to be no known compensating pathway for the absence of the other enzyme. It is possible that among the large number of unidentified X. fastidiosa genes there are non-homologous genes that compensate for steps in such critical pathways. However, barring this possibility, the absence of a functional gluconeogenesis pathway implies a strict dependence on carbohydrates both as a source of energy and anbolic precursors. The glyoxylate cycle is absent and the pentose phosphate pathway is incomplete. In the latter, genes for neither 6-phosphogluconic dehydrogenase nor transaldolase were identifiable. Small molecule metabolism. X. fastidiosa exhibits extensive biosynthetic capabilities, presumably an absolute requirement for a xylem-dwelling bacterium. Most of the genes found in E. coli necessary for the synthesis of all amino acids from chorismate, pyruvate, 3-phosphoglycerate, glutamate and oxaloacetic acid (Blattner, et al, supra) were identified. However, some genes in X. fastidiosa are bi-functional, such as: phosphoribosyl-AMP cyclohydrolase/phosphoribosyl-ATP pyrophosphatase (XF2213), aspartokinase/homoserine dehydrogenase I (XF2225), imidazoleglycerolphosphate dehydratase/histidinol-phosphate phosphatase (XF2217) and a new diaminopimelate decarboxylase/aspartate kinase (XF1116) that would catalyse the first and the last steps of lysine biosynthesis. In addition, the gene for acetylglutamate Kinase (XF1001) has an acetyltransferase domain at its carboxyl-terminal end that would compensate for the missing acetyltransferase in the arginine biosynthesis pathway. Other missing genes include phosphoserine phosphatase, cystathionine β-lyase, homoserine O-succinyltransferase and 2,4,5-methyltetrahydrofolate-homocysteine methyltransferase. The first two enzymes are also absent in the Bacillus subtilis genome, the third is absent in Haemophilus influenzae and the fourth is missing in both genomes (Tatusov, et al, supra). It is presumed that alternative, as yet unidentified enzymes complete the biosynthetic pathways in these organisms as well as in X. fastidiosa.

The pathways for the synthesis of purines, pyrimidines and nucleotides are all complete. In addition, X. fastidiosa is apparently capable of both synthesizing and elongating fatty acids from acetate. Again, however, some enzymes present in E. coli were not found, such as holo acyl-carrier-protein synthase (also absent in Synechocystis sp., H. influenzae and Mycoplasma genitalium) and enoyl-ACP reductase (NADPH) (FabI) (also absent from M. genitalium, Borrelia burgdorferi and Treponema pallidum).

Xylella fastidiosa appears to be capable of synthesizing an extensive variety of enzyme cofactors and prosthetic groups including biotin, folic acid, pantothenate and coenzyme A, ubiquinone, glutathione, thioredoxin, glutaredoxin, riboflavin, FMN, FAD, pyrimidine nucleotides, porphyrin, thiamin, pyridoxal 5′-phosphate and lipoate. In a number of the synthetic pathways, one or more of the enzymes present in E. coli are absent, but this is also true for at least one other sequenced Gram-negative bacterial genome in each case (Tatusov, et al, supra . Again, it is inferred that the missing enzymes are either not essential or are replaced by unknown proteins with novel structures.

Transport-related proteins. A total of 140 genes encoding transport-related proteins were identified, representing 4.8% of all ORFs. For comparison, E. coli, B. subtilis and M. genitalium have around 10% of genes encoding transport proteins, while Helicobacter pylori, Synechocystis sp. and Methanoococcus jannaschhii have 3.5 to 5.4% (Paulsen, et al, J. Mol. Biol. 277:573-592 (1998)). Transport systems are central components of the host-pathogen relationship depicted in FIG. 2. There are a number of ion transporters as well as transporters for the uptake of carbohydrates, amino acids, peptides, nitrate/nitrite, sulphate, phosphate and vitamin B12. Many different transport families are represented and include both small and large mechanosensitive conductance ion channels, a monovalent cation:proton antiporter (CAP-2) and a glycerol facilitator belonging to the major intrinsic protein (MIP) family. In addition, 23 ABC transport systems, comprising 41 genes can be identified. Xylella fastidiosa appears to possess a phosphotransferase system (PTS) that typically mediates small carbohydrate uptake. Both the enzyme I and Hpr components of this system are present, as well as a gene supposedly involved in its regulation (pstK or hprK); however no PTS permease, an essential component of the phosphotransferase complex, was found. The functionality of the system thus remains in question.

There are five outer membrane receptors, including siderophores, ferrichromeiron and hemin receptors that are all associated with iron transport. The energizing complexes, TonB-ExbB-ExbD and the paralogous TolA-TolR-TolQ, essential for the functioning of the outer membrane receptors, are also present. In all, some 67 genes encode proteins involved in iron metabolism. It is proposed herein that in X. fastidiosa the uptake of iron and possibly of other transition metal ions such as maganese causes a reduction in essential micronutrients in the plant xylem, contributing to the typical symptoms of leaf variegation.

The X. fastidiosa genome encodes a battery of proteins that mediate drug inactivation and detoxification, alteration of potential drug targets, prevention of drug entry as well as active extrusion of drugs and toxins. These include ABC transporters and transport processes driven by a proton gradient. Of the latter, eight belong to the hydrophobe/amphiphile efflux-1 (HAE1) family, which act as multidrug resistance factors.

Adhesion. Xylella fastidiosa is characteristically observed embedded in an extracellular translucent matrix in planta (Chagas, et al, J. Phytopathol 134:306-312 (1992)). Clumps of bacteria form within the xylem vessels leading to their blockage and symptoms of the disease such as water-stress leaf curling. We deduce, from our analysis of the complete genome sequence, that the matrix is composed of extracellular polysaccharides (EPSs) synthesized by enzymes closely related to those of Xanthomonas campestris pv campestris (Xcc) that produce what is commercially known as xanthan gum. In comparison with Xcc, however, gumI, encoding glycosyltransferase V (which incorporates the terminal mannose), gumL (encoding ketalase which adds pyruvate to the polymer) and gumG (encoding acetyltransferase which adds acetate) were not found, suggesting that Xylella gum may be somewhat less viscous than its Xanthomonas counterpart.

Positive regulation of the synthesis of extracellular enzymes and EPS in Xanthomomomas is effected by proteins coded by the rpf (regulation of pathogenicity factors) gene cluster. Mutations in any of these genes in Xanthomomas results in failure to synthesize the EPS. In consequence the strain becomes non-pathogenic (Tang, et al, Mol. Gen. Genet 226:409-417 (1991)). Xylella fastidiosa contains genes that encode RpfA, RpfB, RpfC, and RpfF, suggesting that both bacteria may regulate the synthesis of pathogenic EPS factors via similar mechanisms.

Fimbria-like structures are readily apparent upon electron microscopical observation of X. fastidiosa within both its plant and insect hosts (Raju, et al, Plant Disease 70:182-186 (1986)). Because of the high velocity of xylem sap passing through narrow portions of the insect foregut, fimbriae-mediated attachment may be essential for insect colonization. Indeed, within the insect mouthparts, the bacteria are attached in ordered arrays indicating specific and polarized adhesion (Brlansky, et al, Phytopathology 73: 530-535 (1983)). In addition, fimbriae are suspected of playing a role both in plant-bacterium and bacterium-bacterium interactions during colonisation of the xylem itself. Twenty-six genes encoding proteins responsible for the biogenesis and function of Type-4 fimbriae filaments were identified. This type of fimbria is found at the poles of a wide range of bacterial pathogens where they act to mediate adhesion and translocation along epithelial surfaces (Fernandez, et al, FEMS Microbol. Rev 24:21-44 (2000)). The genes include pilS and pilR homologs, which encode a two-component system controlling transcription of fimbrial subunits, presumably in response to host cues, as well as pilG, H, I, J, and chpA, which encode a chemotactic system transducing environmental signals to the pilus machinery.

In addition to the EPS and fimbriae, likely to play central roles in the clumping of bacteria as well as adhesion to the xylem walls, we also identified outer membrane protein homologues for a fimbrial adhesins. Although fimbrial adhesins are well characterised as crucial virulence factors in both plant and human pathogens (Soto, et al, J. Bacteriol 181:1059-1071 (1999)), afimbrial adhesins, which are directly associated with the bacterial cell surface, have been hitherto associated only with human and animal pathogens where they promote adherence to epithelial tissue. Of the three putative adhesins of this kind identified in X. fastidiosa, two exhibit significant similarity to each other (XF1981 and XF1529) and to the hsf and hia gene products of H. influenzae (Geme, et al, Am. J. Respir. Crit. Care Med 154:5192-5196 (1996)). The third (XF1516) is similar to the uspA1 gene product of Moraxella catarrhalis (Cope, et al, J. Bacteriol 181:4026-4034 (1999)). All these proteins share the common C-terminal domain of the autotransporter family (Henderson, et al, Trends Microbiol 6:370-378 (1998)). Direct experimentation will be required to establish whether these adhesins promote binding to plant cell structures or components of the insect vector foregut, or both. Nevertheless, their presence in the X. fastidiosa genome adds to the increasing evidence for the generality of mechanisms of bacterial pathogenicity, irrespective of the host organism (Rahme, et al, Science 268:1899-1902 (1995)).

Three different hemagglutinin-like genes were also identified. Again, similar genes have not previously been identified in plant pathogens. These genes (XF2775, XF2196 and XF0889) are the largest in the genome and exhibit highest similarity to a Neisseria meningitidis putative secreted protein (Tettelin, et al, Science 287:1809-1815 (2000)).

Intervessel migration. Movement between individual xylem vessels is crucial to effective colonization by X. fastidiosa. For this to occur, degradation of the pit membrane of the xylem vessel is required. Of known pectolytic enzymes capable of this function, a polygalacturonase precursor and a cellulase were identified, although the former contains an authentic frameshift. These genes exhibited highest similarity to orthologues in Ralstonia solanaceaium, which causes wilt disease in tomatoes, and where the polygalacturonase genes are required for wild-type virulence.

Toxicity. Five hemolysin-like genes were identified. One is hemolysin III (XF0175) belonging to an uncharacterised protein family, and four others (XF0668, XF1011, XF2407 and XF2759) belong to the RTX toxin family that contains tandemly repeated glycine-rich nonapeptide motifs at the C-terminal domain. One of these ORFs is closely related to bacteriocin, an RTX toxin also present in the plant bacterium Rhizobium leguminosarum (Oresnik, et al, Appl. Environ., Microbiol 65:2833-2840 (1999)). RTX or RTX-like proteins are important virulence factors widely distributed among gram-negative pathogenic bacteria (Lally, et al, Trends Microbiol 7:356-361 (1999).

There are two Colicin V-like precursor proteins. Colicin V is an antibacterial polypeptide toxin produced by E. coli, acting against closely related sensitive bacteria (Havarstein, et al, Microbiology 140: 2383-2389 (1994). The precursors consist of 102-amino-acid-long peptides (XF0262, XF0263) that have the typical conserved leader 15-amino acid motif, and present some similarity with Colicin V from E. coli at the remaining C-terminal portion. The necessary apparatus for Colicin biosynthesis and secretion is also present. Interestingly, in E. coli most of the necessary genes for biogenesis and export of Colicin V are in a gene cluster present in a plasmid, whereas in X. fastidiosa these genes are dispersed in the chromosome.

Four genes that may function in polyketide biogenesis were found: polyketide synthase (PKS), pteridine-dependent deoxygenase, daunorubicin C-13 ketoreductase and a NonF-related protein. These genes belong to the synthesis pathways of frenolicin, rapamycin, daunorubicin and nonactin, respectively. These pathways include many more enzymes, which were not found. On the other hand, some of the genes listed lie close to ORFs without significant database matches, suggesting that at least one (as-yet undiscovered) polyketide pathway may be functional.

Prophages. Bacteriophages can mediate the evolution and transfer of virulence factors and occasional acquisition of new traits by the bacterial host. Since as much as 7% of the X. fastidiosa genome sequenced corresponds to dsDNA phage sequences, mostly from the Lambda group, it is suspected that this route may have been of particular importance for this bacterium. It is noteworthy that a very high percentage of phage-related sequences has also been detected in a second vascular-restricted plant pathogen, Spiroplasma citri (Ye, et al, Nucleic Acids Res 20:1554-1565 (1992)). Four regions were identified, which have a high density of ORFs homologous to phage sequences, that are considered to be prophages, in addition to isolated phage sequences dispersed throughout the genome. Two of these prophages, of about 42 kbp each, nominated as XfP1 and XfP2, are similar to each other, lie in opposite orientations in distinct regions and appear to belong to the dsDNA, tailed-phage group. Both appear to contained most of the genes responsible for particle assembly, although no reports of phage particle release from X. fastidiosa cultures are known to the inventors. In prophage XfP1, between tail genes V and W, two ORFs were found that are similar to ORF118 and vapA from the virulence-associated region of the animal pathogen Dichelobacter nodosus, which by homology encode a killer and a suppressor protein (Billington, et al, FEMS Microbiol Let. 145:147-156 (1996)). Interestingly, in prophage XfP2, also between tail genes V and W, two other ORFs were found that are similar to hypothetical ORFs of Ralstonia eutropha transposon Tn4371 (Merlin, et al, Plasmid 41:40-54 (1999)). The other two identified prophages, XfP3 and XfP4, are also similar in sequence to each other and to the H. influenzae cryptic prophage Φflu (Hendrix, et al, Proc. Natl. Acad. Sci USA 96:2192-2197 (1999)). They both contain a 14,317 bp-long exact repeat. Few particle-assembly genes were found in these regions, suggesting that these prophages are defective. An ORF similar to hicB from H. influenzae, a component of the major pilus gene cluster in some isolates, was found in XfP4.

The presence of virulence-associated genes from other organisms within the prophage sequences is strong evidence for a direct role for bacteriophage-mediated horizontal gene transfer in the definition of the bacterial phenotype.

Absence of avirulence genes. Phytopathogenic bacteria generally have a limited host range, often confined to members of a single species or genus. This specificity is defined by the products of the so-called avirulence (avr) genes present in the pathogen, which are injected directly into host cells, on infection, via a type III secretory system (Alfano, et al, J. Bacteriol 179:5655-5662 (1997); Galan, et al, Science 284:1322-1328 (1999); Young, et al, Proc. Natl. Acad. Sci USA 96:6456-6461 (1999)). BLAST (Altschul, et al, Nucl. Acids Res 25:3389-3402 (1997)) searches with all known avr and type III secretory system sequences failed to identify genes. encoding proteins with significant similarities in the genome of X. fastidiosa. Although the variability of avr genes amongst bacteria could account for this apparent lack, the high level of similarity of some components of the type III secretory system argues against this. We suspect that these genes are, in fact, not required due to the insect-mediated tranmission and vascular restriction of the bacterium that obviates the necessity of host cell infection. Furthermore, if the differing host ranges of X. fastidiosa are moleculary defined, this may be by a quite different mechanism not involving avr proteins.

TABLE 1
General features of the Xylella fastidiosa 9a5c genome.

| Main Chromosome | |
|---|---|
| Length (bp) | 2,679,305 |
| G + C ratio | 52.7% |
| Open Reading Frames (ORFs) | 2,782 |
| Coding region (as % of chromosome size) | 88.0% |
| Average ORF length | 799 bp |
| ORFs with functional assignment | 1,283 |
| ORFs with matches to conserved hypothetical | 310 |
| proteins | |
| ORFs without significant data base match | 1,083 |
| Ribosomal RNA operons | 2 |
| | (16SrRNA-Ala-TGC- |
| | tRNA-Ile-GAT-tRNA- |
| | 23SrRNA-5SrRNA) |
| tRNAs | 49 (46 different |
| | sequences corresponding |
| | to all 20 amino acids) |
| tmRNA | 1 |
| Plasmid pXF51 | |
| Length (bp) | 51,158 |
| G + C ratio | 49.6% |
| Open Reading Frames (ORFs) | 64 |
| Protein coding region (as % of plasmid size) | 86.9% |
| ORFs with functional assignment | 30 |
| ORFs with matches to conserved hypothetical | 8 |
| proteins | |
| ORFs without significant data base match | 24 |
| Plasmid pXF1.3 | |
| Length (bp) | 1,285 |
| G + C ratio | 55.6% |
| Open Reading Frames (ORFs) | 2 |
| ORFs with functional assignment | 1 |

TABLE 2
Largest families of paralogous genes.

| Family | Number of genes |
|---|---|
| (total number of families = 312) | (total number of genes = 853) |
| ATP-binding subunits of ABC transporters | 23 |
| reductases/dehydrogenases | 12 |
| two-component system, regulatory | 12 |
| proteins | |
| hypothetical proteins | 10 |
| transcriptional regulators | 9 |
| fimbrial proteins | 9 |
| two-component system, sensor proteins | 9 |

The foregoing disclosure sets forth various features of the invention which include, e.g., an isolated nucleic acid molecule, the nucleotide sequence of which is set forth at SEQ ID NO: 1, as well as smaller nucleic acid molecules, such as those whose nucleotide sequence consists of a nucleotide sequence defined by one of the arrows in FIG. 1. Such nucleotide sequences encode for specific Xyllela fastidiosa proteins, as set forth at, e.g., table 3. These proteins are also a part of the invention, as are degenerate sequences which encode the same protein.

Another aspect of the invention are computer readable media which have recorded thereon all or a part of the nucleotide sequence of SEQ ID NO: 1, or degenerate variants thereof. Exemplary of such computer readable media are floppy discs, hard discs, random access memory (RAM), read only memory (ROM), and CD-ROMs. Such computer readable media are useful, e.g., in identifying whether a nucleic acid molecule of interest is from, or is homologous to, a Xyllela fastidiosa nucleic acid molecule. Thus, they permit the skilled artisan to determine if a plant, plant part, or collection of plants, such as an orchard, are infected with Xyllela fastidiosa, or if an organism is potentially pathogenic to plants, based upon homology to X. fastidiosa.

Other aspects of the invention will be clear to the skilled artisan and need not be set forth herein.

The terms and expressions which have been employed herein are used as terms of description and not of limitation, and there is no intention in the use of such terms and expressions of excluding any equivalents of the features shown and described or portions thereof, it being recognized that various modifications are possible within the scope of the invention.

We claim:

1. Computer readable medium having recorded thereon a nucleic acid molecule from X. fastidiosa genome.

2. The computer readable medium of claim 1, consisting of a floppy disc, a hard disc, random access memory, read only memory, or CD-ROM.

3. The computer readable medium of claim 1, wherein said nucleic acid molecule is from X. fastidiosa chromosome.
